(19) United States
(12) Patent Application Publication
(10) Pub. No.: US 2025/0108048 A1
(43) Pub. Date: Apr. 3, 2025

(54) STING AGONIST COMPOUNDS AND CONJUGATES

(71) Applicant: SUTRO BIOPHARMA, INC., South San Francisco, CA (US)

(72) Inventors: Ravi Prakash SINGH, Burlingame, CA (US); Huiyong HU, Fremont, CA (US); Wen LIU, Foster City, CA (US); Krishna BAJJURI, Union City, CA (US); Xiangwei ZHU, Albany, CA (US)

(21) Appl. No.: 18/789,214

(22) Filed: Jul. 30, 2024

Related U.S. Application Data
(60) Provisional application No. 63/653,388, filed on May 30, 2024.
(60) Provisional application No. 63/516,579, filed on Jul. 31, 2023.

(51) Int. Cl. A61K 31/4985; A61K 31/4184; A61K 31/5386; A61K 38/16; A61P 35/00; C07D 235/30; C07D 487/04; C07D 498/10

(52) U.S. Cl. CPC A61K 31/4985; A61K 31/4184; A61K 31/5386; A61K 38/16; A61P 35/00; C07D 235/30; C07D 487/04; C07D 498/10

(57) ABSTRACT

The present disclosure is related to STING agonists, linker-payloads thereof, and conjugates thereof, pharmaceutical compositions thereof, and the use of the agonists, conjugates, and pharmaceutical compositions to induce a STING-mediated immune response and/or for the treatment of diseases and disorders mediated by STING.

### CROSS REFERENCE

The present application claims the benefit of US provisional application nos. 63/516,579, filed Jul. 31, 2023, and 63/653,388, filed May 30, 2024, the contents of which are hereby incorporated by reference in their entireties.

### FIELD

The present disclosure is related to STING agonists and conjugates, pharmaceutical compositions thereof, and the use of the agonists, conjugates, and pharmaceutical compositions to induce a STING-mediated immune response and/or for the treatment of diseases and disorders mediated by STING.

### BACKGROUND

The innate and adaptive immune systems work closely together to fight foreign substances and invading pathogens. While the adaptive system is highly specific and long-lasting due to the production of memory T-cells, the innate system acts quickly as the body's first line of defense. The innate system responds non-specifically to both pathogen-derived cytosolic DNA and host cytosolic DNA. In doing this, the innate immune system not only provides broad protection against threats such as bacteria and viruses, but also responds to signals of cellular and tissue damage.

One protein that is important for innate immunity is stimulator of interferon genes (STING), and the cGAS-STING pathway in particular helps to sense and protect against harmful cytosolic DNA. cGAS recognizes cytosolic DNA and catalyzes the synthesis of cyclic dinucleotides (CDNs), including cGAMP, which in turn bind and activate STING. Once STING is bound to a CDN, STING undergoes a conformational change, translocates from the endoplasmic reticulum to the Golgi apparatus, and triggers the transcription factor TBK1 to phosphorylate transcription factors interferon regulatory factor 3 (IRF3) and nuclear factor κB (NF-κB). This induces type I interferons (IFNs) and the production of pro-inflammatory cytokines, such as IL-6, TNF-α, and IFN-γ.

Various DNA viruses have been shown to activate the STING pathway via this mechanism, including the herpes simplex virus type I (HSV-1), Kaposi sarcoma herpes virus (KSHV), cytomegalovirus (CMV), hepatitis B (HBV), human papillomavirus (HPV), adenoviruses, and baculoviruses. Studies have also shown that STING can protect against RNA infections. For example, STING knockout mice are more susceptible to RNA viruses (Ishikawa, H., et al. Nature, 2009, 461, pages 788-792). Additionally, intracellular bacteria have been shown to produce CDNs that can activate the STING pathway and induce an immune response. For example, the bacteria strain Listeria monocytogenes elicits a STING-induced immune response.

While foreign agents can activate the STING pathway, many viruses have developed methods to suppress or inhibit the STING-promoted immune response. For example, a number of HSV-1 viral genes are capable of suppressing STING signaling pathways, including HSV-1 γ34.5, which disrupts STING trafficking from the endoplasmic reticulum to the Golgi apparatus (Christensen, M. H., et al., EMBO, 2016, 35, 568). Similarly, it has also been demonstrated that Kaposi sarcoma herpes virus (KSHV), human papillomavirus (HPV), cytomegalovirus (CMV), and hepatitis B (HBV) viral genes have developed mechanisms for evading the STING pathway. (Ahn, J. et al. Experimental and Molecular Medicine, 2019, 51, 155).

Furthermore, certain RNA viruses affect IFN production. For instance, it has been reported that the protease of Dengue Fever (DENV), a single-positive-stranded RNA virus, can inhibit type I IFN production by targeting and cleaving STING. In STING-deficient primary cells, DENV replication is highly increased (Yu, C. et al. PLoS Pathog. 2012, 8, e1002780; Aguirre, S., et al. PLoS Pathog. 2012, 8, e1002934). Similarly, the protease for Zika virus (ZIKV), another single-positive-stranded RNA virus of the same Flaviviridae family, can also cleave STING and reduce type I IFN production through downstream effects (Ding, et al. Proc. Natl. Acad. Sci. USA 2018, 115, E6310; Zheng et al. EMBO, 2018, 37: e99347).

In addition to helping mount an immune response against foreign pathogens, the STING pathway also recognizes host-cytosolic DNA. The cytosol is normally free of DNA so leaked cytosolic DNA is often an indication of DNA damage events and tumorigenesis. Detection of host-cytosolic DNA by STING leads to the production of IFNs, immune-stimulated genes, and pro-inflammatory cytokines. It has been well-established that IFNs can inhibit tumor cell proliferation via multiple mechanisms. As described in Jiang, M. et al. Journal of Hematology & Oncology, 2020, 81, 13, a STING-deficiency is correlated with cancer incidence at least in melanoma cell lines, colorectal adenocarcinoma human cell lines, and lung cancer.

A number of STING agonists have been developed and studied for oncological indications (Le Naour et al. Oncoimmunology, 2020; 9(1): 1777624), including DMXAA (or Vadimezan), a tumor-vascular disrupting agent that has been studied in clinical trials for its effect on advanced solid tumors, prostate cancer, urothelial carcinoma, and small cell lung cancer. Despite promising preclinical results, DMXAA has thus far only yielded poor results in human clinical trials. MIW815 (ADU-S100) in combination with pembrolizumab was recently studied in a Phase 2 clinical trial for patients with head and neck cancer, but the trial was terminated due to a lack of substantial anti-tumor activity (NCT03937141). A Phase 1 trial to study the effect of MIW815 as a single agent and in combination with ipilimumab in patients with advanced/metastatic solid tumors or lymphomas (NCT02675439) was also terminated for showing a lack of substantial anti-tumor activity.

Other STING agonists include diamidobenzimidazole (di-ABZI) STING agonists, for example, those described in U.S. Pat. No. 11,377,440. U.S. Pat. No. 11,155,567 describes di-ABZI STING agonists, including XMT-2056, however, a Phase I clinical trial of XMT-2056 for HER2+ recurrent or metastatic solid tumors was suspended in March 2023 following a patient death (NCT05514717). Additional di-ABZI STING agonists are described in PCT Applications WO 2020/042995; WO 2020/156363; WO 2023/025256; WO 2021/013250; and WO 2022/272039.

Given the importance of the STING pathway in inducing an immune response both in response to foreign pathogens and damaged DNA associated with cancer, there is a medical need to develop STING agonists. It is therefore an object of the present disclosure to provide novel compounds, conjugates, and compositions that can induce a STING-mediated immune response and/or provide treatment of diseases and disorders mediated by STING, such as cancer.

### SUMMARY

In one aspect, provided herein is a compound of Formula (I):

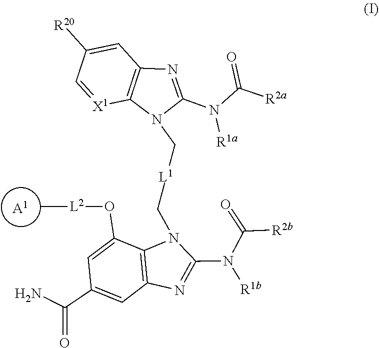

or a pharmaceutically acceptable salt or tautomer thereof,

wherein

X¹ is selected from N and CR³;

R²⁰ is selected from hydrogen and —CON(R^{3a})(R^{3b});

R^{1a}, R^{1b}, R^{3a} and R^{3b} are independently selected from hydrogen and optionally substituted C₁₋₆ alkyl, wherein the C₁₋₆ alkyl is optionally substituted with one or more R⁵⁰;

R^{2a} and R^{2b} are independently selected from: (a) optionally substituted C₁₋₆ alkyl, wherein the C₁₋₆ alkyl is optionally substituted with one or more R⁵¹ and (b) C₃₋₁₂ carbocycle and 3- to 12-membered heterocycle, each of which is optionally substituted with one or more R⁵³;

or R^{1a} and R^{2a} are joined together with the atoms to which they are attached to form an optionally substituted 3- to 12-membered heterocycle, wherein the heterocycle is optionally substituted with one or more R⁵3;

or R^{1b} and R^{2b} are joined together with the atoms to which they are attached to form an optionally substituted 3- to 12-membered heterocycle, wherein the heterocycle is optionally substituted with one or more R⁵3;

R³ is selected from hydrogen, —OR³⁰, —SR³⁰, —C(O)N(R³⁰)₂, —N(R³⁰)C(O)R³⁰, —N(R³⁰)C(O)N(R³⁰)₂, —N(R³⁰)₂, —C(O)R³⁰, —C(O)OR³⁰, —OC(O)R³⁰, —NO₂, and —CN;

L¹ is selected from a bond, —C₁₋₁₀alkylene-, —C₂₋₁₀alkenylene-, —C₂₋₁₀alknyene-, —C₁₋₆alkylene-O—C₁₋₆alkylene-, —C₁₋₆alkylene-NC₁₋₆alkylene-, C₃₋₆carbocycle, and —C₁₋₆alkylene-(C₃₋₆carbocycle)-C₁₋₆alkylene-, wherein the —C₁₋₁₀alkylene-, —C₂₋₁₀alkenylene-, —C₂₋₁₀alkynylene-, and each —C₁₋₆alkylene group of —C₁₋₆alkylene-O—C₁₋₆alkylene-, —C₁₋₆alkylene-NH—C₁₋₆alkylene-, and —C₁₋₆alkylene-(C₃₋₆carbocycle)-C₁₋₆alkylene- are optionally substituted with one or more R⁵⁰;

L² is optionally substituted —C₁₋₆ alkylene-, wherein the —C₁₋₆ alkylene- is optionally substituted with one or more R⁵⁰;

Ring A¹ is selected from (a) an optionally substituted bridged, fused, or spirocyclic bicyclic heterocycle comprising at least one N atom and at least one 0 atom, wherein the heterocycle is optionally substituted with one or more R⁵³; and (b) a 3- to 12-membered heterocycle substituted with R⁴;

R⁴ is an optionally substituted 3- to 12-membered heterocycle, optionally comprising at least one NR⁵ and wherein the heterocycle is optionally substituted with one or more R⁵3;

R⁵ is selected from hydrogen, R⁶, —C(O)—C₁₋₆alkyl, —C(O)-heteroC₁₋₆alkyl, C₁₋₆ alkyl, and heteroC₁₋₆alkyl wherein the C₁₋₆ alkyl, either alone or part of another group, is optionally substituted with one or more R⁵⁰;

R⁶ is an amino acid residue;

each R³⁰ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more R⁵;

R⁵⁰ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, C₃₋₁₂ carbocycle, and 3- to 12-membered heterocycle;

R⁵¹ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, optionally substituted C₃₋₁₂ carbocycle, and optionally substituted 3- to 12-membered heterocycle, wherein said optionally substituted C₃₋₁₂ carbocycle and optionally substituted 3- to 12-membered heterocycle are optionally substituted with one or more R⁵².

R⁵² is independently selected at each occurrence from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), —CN, C₁₋₆ alkyl, C₂₋₆ alkenyl, and C₂₋₆ alkynyl;

R⁵³ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, an amino acid residue, optionally substituted C₁₋₆ alkyl, optionally substituted C₂₋₆ alkenyl, optionally substituted C₂₋₆ alkynyl, optionally substituted C₃₋₁₂ carbocycle, and optionally substituted 3- to 12-membered heterocycle, wherein said optionally substituted C₁₋₆ alkyl, optionally substituted C₂₋₆ alkenyl, and optionally substituted C₂₋₆ alkynyl are optionally substituted with one or more R⁵⁴ and said optionally substituted C₃₋₁₂ carbocycle and optionally substituted 3- to 12-membered heterocycle are optionally substituted with one or more R⁵²;

R⁵⁴ is independently selected at each occurrence from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), and —CN;

R⁵⁵ is independently selected at each occurrence from halogen, —CN, —NO₂, —OH, —N(R⁶⁰)₂, —C(O)N(R⁶⁰)₂, ═O, ═S, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;

each R⁶⁰ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle; and

each R⁶¹ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle.

In another aspect, provided herein is a compound of Formula (II):

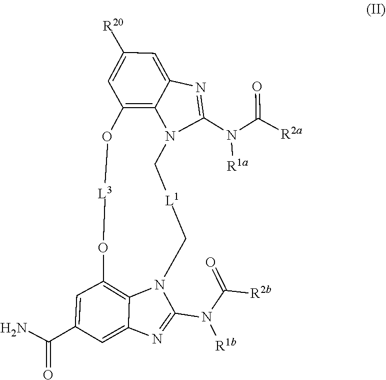

or a pharmaceutically acceptable salt or tautomer thereof,

wherein

R²⁰ is selected from hydrogen and —CON(R^{3a})(R^{3b});

R^{1a}, R_{1b}, R^{3a} and R^{3b} are independently selected from hydrogen and optionally substituted C₁₋₆ alkyl, wherein the C₁₋₆ alkyl is optionally substituted with one or more R⁵⁰;

R^{2a} and R^{2b} are independently selected from: (a) optionally substituted C₁₋₆ alkyl, wherein the C₁₋₆ alkyl is optionally substituted with one or more R⁵¹ and (b) C₃₋₁₂ carbocycle and 3- to 12-membered heterocycle, each of which is optionally substituted with one or more R⁵³;

or R^{1a} and R^{2a} are joined together with the atoms to which they are attached to form an optionally substituted 3- to 12-membered heterocycle, wherein the heterocycle is optionally substituted with one or more R⁵3;

or R^{1b} and R^{2b} are joined together with the atoms to which they are attached to form an optionally substituted 3- to 12-membered heterocycle, wherein the heterocycle is optionally substituted with one or more R⁵³;

L¹ is selected from a bond, —C₁₋₁₀alkylene-, —C₂₋₁₀alkenylene-, —C₂₋₁₀alkynylene, —C₁₋₆alkylene-O—C₁₋₆alkylene-, —C₁₋₆alkkylene-NH—C₁₋₆alkylene-, C₃₋₆carbocycle, and —C₁₋₆ alkylene-((C₃₋₆carbocycle)-C₁₋₆alkylene-, wherein the —C₁₋₁₀ aalkylene-, —C₂₋₁₀alkenylene, —C₂₋₁₀alkynylene-, and each C₁₋₆ alkylene group of —C₁₋₆alkylene-O—C₁₋₆alkylene-, —C₁₋₆alkylene-NH—C₁₋₆alkylene, and —C₁₋₆alkylene-(C₃₋₆carbocycle)-(C₁₋₆alkylene- are opdlonally substituted with one or more R⁵⁰;

L³ is C₁₋₆ alkylene, which is substituted with either 1) one or more R⁵⁰ or 2) R^{8a} and R^{8b}, wherein

when L³ is substituted with R^{8a} and R^{8b}, R^{8a} and R^{8b} are joined together with the atoms to which they are attached to form an optionally substituted C₃₋₁₂ carbocycle or an optionally substituted 3- to 12-membered heterocycle, wherein said optionally substituted C₃₋₁₂ carbocycle and optionally substituted 3- to 12-membered heterocycle are optionally substituted with one or more R⁵²;

R⁵⁰ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, C₃₋₁₂ carbocycle, and 3- to 12-membered heterocycle;

R⁵¹ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, optionally substituted C₃₋₁₂ carbocycle, and optionally substituted 3- to 12-membered heterocycle, wherein said optionally substituted C₃₋₁₂ carbocycle and optionally substituted 3- to 12-membered heterocycle are optionally substituted with one or more R⁵²;

R⁵² is independently selected at each occurrence from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), —CN, C₁₋₆ alkyl, C₂₋₆ alkenyl, and C₂₋₆ alkynyl;

R⁵³ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, an amino acid residue, optionally substituted C₁₋₆ alkyl, optionally substituted C₂₋₆ alkenyl, optionally substituted C₂₋₆ alkynyl, optionally substituted C₃₋₁₂ carbocycle, and optionally substituted 3- to 12-membered heterocycle, wherein said optionally substituted C₁₋₆ alkyl, optionally substituted C₂₋₆ alkenyl, and optionally substituted C₂₋₆ alkynyl are optionally substituted with one or more R⁵⁴ and said optionally substituted C₃₋₁₂ carbocycle and optionally substituted 3- to 12-membered heterocycle are optionally substituted with one or more R⁵²;

R⁵⁴ is independently selected at each occurrence from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), and —CN;

each R⁶⁰ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle; and

each R⁶¹ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle.

In another aspect, provided herein is a compound of Formula (III):

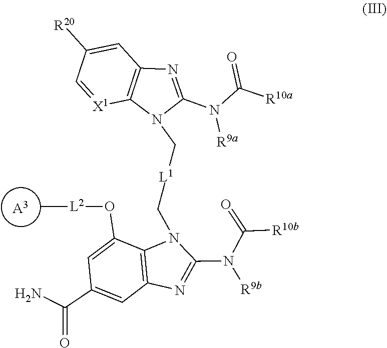

or a pharmaceutically acceptable salt thereof or tautomer thereof,

wherein

X¹ is selected from N and CR³;

R³ is selected from H, —OR³⁰, —SR³⁰, —C(O)N(R³⁰)₂, —N(R³⁰)C(O)R³⁰, —N(R³⁰)C(O)N(R³⁰)₂, —N(R³⁰)₂, —C(O)R³⁰, —C(O)OR³⁰, —OC(O)R³⁰, —NO₂, and —CN;

R^{3a} and R^{3b} are independently selected from hydrogen and optionally substituted C₁₋₆ alkyl, wherein the C₁₋₆ alkyl is optionally substituted with one or more R⁵⁰;

R^{9a} and R^{10a} are joined together with the atoms to which they are attached to form an optionally substituted 3- to 12-membered heterocycle, wherein the heterocycle is optionally substituted with one or more R⁵³;

R^{9b} and R^{10b} are joined together with the atoms to which they are attached to form an optionally substituted 3- to 12-membered heterocycle, wherein the heterocycle is optionally substituted with one or more R³;

R²⁰ is selected from hydrogen and —CON(R^{3a})(R^{3b});

each R³⁰ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more R⁵;

L¹ is selected from a bond, —C₁₋₁₀alkylene-, —C₂₋₁₀alkenylene-, —C₂₋₁₀alkynylene-, —C₁₋₆alkylene-O—C₁₋₆alkylene-, —C₁₋₆alkylene-NH—C₁₋₆alkylene-, C₃₋₆carbocycle, and —C₁₋₆alkylene-(C₃₋₆carbocycle)-C₁₋₆alkylene-, wherein the —C₁₋₁₀alkylene-, —C₂₋₁₀alkenylene-, —C₂₋₁₀ (alkynylene-, and each C₁₋₆ alkylene group of —C₁₋₆ alkylene-O—C₁₋₆alkylene —C₁₋₆-alkylene-NH—C₁₋₆alkylene-, and —C₁₋₆alkylene-(C₃₋₆carbocycle)-C₁₋₆ alkylene- are optionally substituted with one or more R⁵⁰;

L² is optionally substituted —C₁₋₆ alkylene-, wherein the —C₁₋₆ alkylene- is optionally substituted with one or more R⁵⁰;

Ring A³ is C₃₋₁₂ carbocycle or 3- to 12-membered heterocycle, each of which is optionally substituted with one or more R⁵3;

R⁵⁰ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, C₃₋₁₂ carbocycle, and 3- to 12-membered heterocycle;

R⁵² is independently selected at each occurrence from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), —CN, C₁₋₆ alkyl, C₂₋₆ alkenyl, and C₂₋₆ alkynyl;

R⁵³ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, an amino acid residue, optionally substituted C₁₋₆ alkyl, optionally substituted C₂₋₆ alkenyl, optionally substituted C₂₋₆ alkynyl, optionally substituted C₃₋₁₂ carbocycle, and optionally substituted 3- to 12-membered heterocycle, wherein said optionally substituted C₁₋₆ alkyl, optionally substituted C₂₋₆ alkenyl, and optionally substituted C₂₋₆ alkynyl are optionally substituted with one or more R⁵⁴ and said optionally substituted C₃₋₁₂ carbocycle and optionally substituted 3- to 12-membered heterocycle are optionally substituted with one or more R⁵²;

R⁵⁴ is independently selected at each occurrence from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), and —CN;

R⁵⁵ is independently selected at each occurrence from halogen, —CN, —NO₂, —OH, —N(R⁶⁰)₂, —C(O)N(R⁶⁰)₂, ═O, ═S, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;

each R⁶⁰ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle; and

each R⁶¹ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle.

In another aspect, provided herein is a compound of Formula (IV):

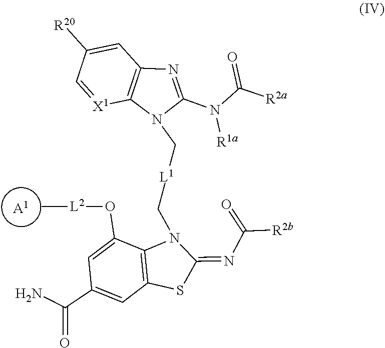

or a pharmaceutically acceptable salt thereof or tautomer thereof,

wherein Ring A¹, L¹, L², X¹, R^{1a}, R^{2a}, R^{2b}, and R²⁰ are as defined herein.

Also provided herein are linker-payloads comprising compounds of Formula (I), Formula (II), Formula (II), or Formula (IV) wherein the compound of Formula (I), Formula (II), Formula (II), or Formula (IV) is linked to a reactive linker group (RG) optionally via a linker.

In one embodiment, the linker-payload is of Formula (LP-I) or Formula (LP-IV):

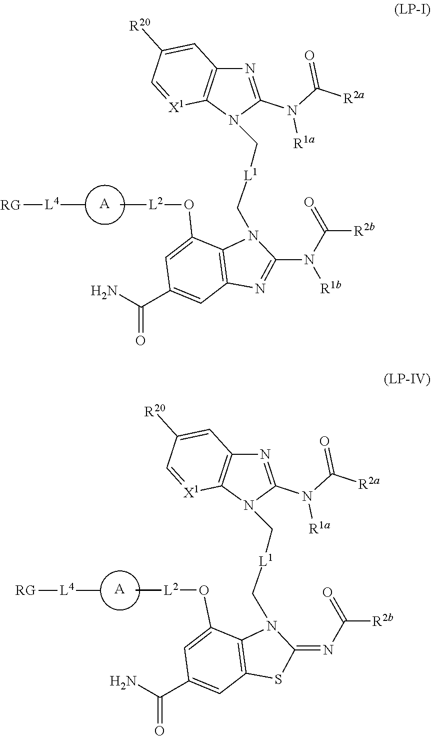

or a pharmaceutically acceptable salt or tautomer thereof,

wherein

L⁴ is a bond or a linker;

RG is a reactive linker group; and

Ring A¹, L¹, L², X¹, R^{1a}, R^{2a}, R^{1b}, R^{2b}, and R²⁰ are as defined herein.

In one embodiment, the linker-payload is of Formula (LP-II):

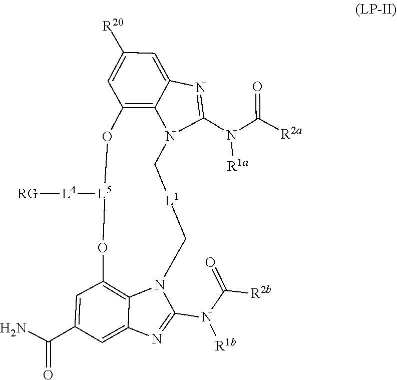

or a pharmaceutically acceptable salt or tautomer thereof,

wherein

L⁵ is a linker comprising —C₁₋₆ alkylene-, which is substituted with either 1) one R⁵⁶ or 2) R^{18a} and R^{18b}; and wherein the —C₁₋₆ alkylene- is optionally further substituted with one or more R⁵⁰; wherein

when L⁵ is substituted with R^{18a} and R^{18b}, R^{18a} and R^{18b} are joined together with the atoms to which they are attached to form a C₃₋₁₂ carbocycle or a 3- to 12-membered heterocycle, wherein said C₃₋₁₂ carbocycle and 3- to 12-membered heterocycle are attached to -L⁴-RG and further optionally substituted with one or more R⁵²;

R⁵⁶ is independently selected at each occurrence from —OR⁶², —SR⁶², —C(O)N(R⁶⁰)(R⁶²), —N(R⁶²)C(O)R⁶⁰, —N(R⁶⁰)C(O)R⁶², —N(R⁶²)C(O)N(R⁶⁰)₂, —N(R⁶⁰)C(O)N(R⁶⁰)(R⁶²), —N(R⁶⁰)(R⁶²), —C(O)R⁶², —C(O)OR⁶², —OC(O)R⁶², =N(R⁶²), C₃₋₁₂ carbocycle substituted with R⁶², and 3- to 12-membered heterocycle substituted with R⁶²;

each R⁶² is independently selected at each occurrence from —C₁₋₁₀ alkylene- attached to -L⁴-RG, —C₂₋₁₀ alkenylene- attached to -L⁴-RG, —C₂₋₁₀ alkynylene- attached to -L⁴-RG, C₃₋₁₂ carbocyclene attached to -L⁴-RG, and 3- to 12-membered heterocyclene attached to -L⁴-RG;

L⁴ is a bond or a linker;

RG is a reactive linker group; and

L¹, R^{1a}, R^{2a}, R^{1b}, R^{2b}, R²⁰, R⁵⁰, and R⁵² are as defined herein.

In one embodiment, the linker-payload is of Formula (LP-III):

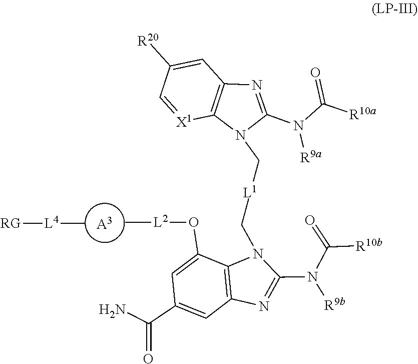

or a pharmaceutically acceptable salt or tautomer thereof,

wherein

L⁴ is a bond or a linker;

RG is a reactive linker group; and

Ring A³, L¹, L², X¹, R^{9a}, R^{10a}, R^{9b}, R_{10b}, and R²⁰ are as defined herein.

In one embodiment, the linker-payload is of Formula (LP-V):

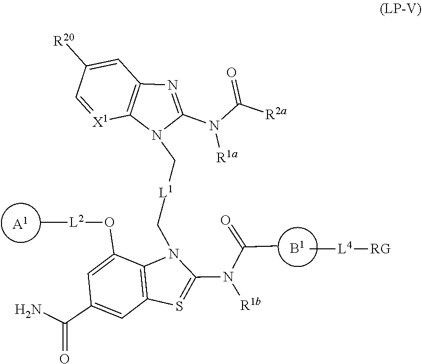

or a pharmaceutically acceptable salt or tautomer thereof,

wherein

Ring B¹ is an optionally substituted C₃₋₁₂ carbocycle or an optionally substituted 3- to 12-membered heterocycle, wherein said optionally substituted C₃₋₁₂ carbocycle and optionally substituted 3- to 12-membered heterocycle are optionally substituted with one or more R⁵2.

L⁴ is a bond or a linker;

RG is a reactive linker group; and

Ring A¹, Ring B¹, L¹, L², X¹, R^{1a}, R^{2a}, R^{1b}, R²⁰, R⁵², and R⁵³ are as defined herein.

In one embodiment, the linker-payload is of Formula (LP-V):

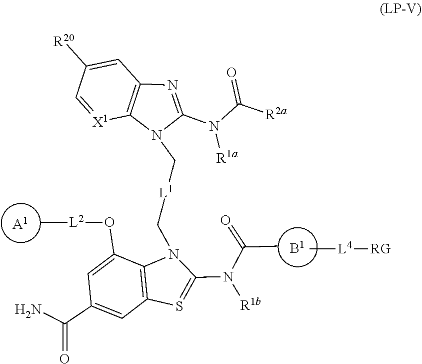

or a pharmaceutically acceptable salt or tautomer thereof,

wherein

Ring B¹ is an optionally substituted C₃₋₁₂ carbocycle or an optionally substituted 3- to 12-membered heterocycle, wherein said optionally substituted C₃₋₁₂ carbocycle and optionally substituted 3- to 12-membered heterocycle are optionally substituted with one or more R³;

L⁴ is a bond or a linker;

RG is a reactive linker group; and

Ring A¹, Ring B¹, L¹, L², X₁, R^{1a}, R^{2a}, R^{1b}, R²⁰, R⁵², and R⁵³ are as defined herein.

In another aspect, provided herein are compound conjugates comprising a compound of Formula (I), Formula (II), Formula (III), or Formula (IV) wherein the compound of Formula (I), Formula (II), Formula (III), or Formula (IV) is linked to a COMP optionally via a linker wherein COMP is a macromolecule. In one embodiment, the COMP is an antibody or antigen binding fragment thereof.

In one embodiment, the compound conjugate is a compound of Formula (CONJ-I):

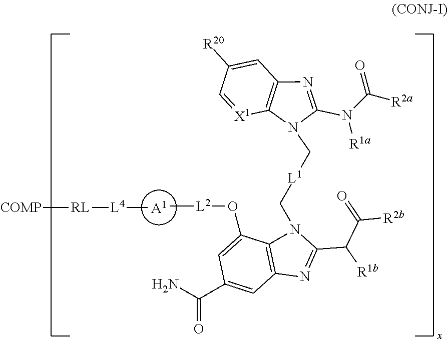

or pharmaceutically acceptable salt or tautomer thereof,

wherein

L⁴ is a bond or a linker;

RL is a reactive linker residue;

x is an integer between 1 and 30, inclusive;

COMP is a macromolecule; and

Ring A¹, L¹, L², X¹, R^{1a}, R^{2a}, R^{1b}, R^{2b}, and R²⁰ are as defined herein.

In one embodiment, the compound conjugate is a compound of Formula (CONJ-II):

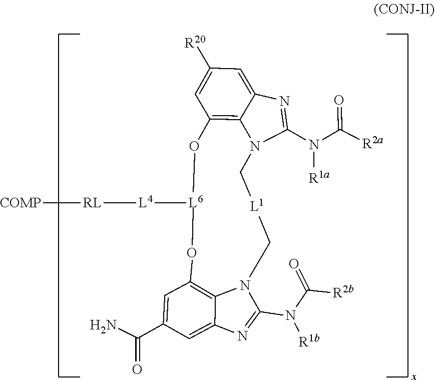

or a pharmaceutically acceptable salt or tautomer thereof;

wherein

L⁶ is a linker comprising —C₁₋₆ alkylene-, which is substituted with either 1) one R⁵⁷ or 2) R^{28a} and R^{28b}; and wherein the —C₁₋₆ alkylene- is optionally further substituted with one or more R⁵⁰; wherein

when L⁶ is substituted with R^{28a} and R^{28b}, R^{28a} and R^{28b} are joined together with the atoms to which they are attached to form a C₃₋₁₂ carbocycle or a 3- to 12-membered heterocycle, wherein said C₃₋₁₂ carbocycle and 3- to 12-membered heterocycle are attached to -L⁴-RL-COMP and further optionally substituted with one or more R⁵2.

R⁵⁷ is independently selected at each occurrence from —OR⁶³, —SR⁶³, —C(O)N(R⁶⁰)(R⁶³), —N(R⁶³)C(O)R⁶⁰, —N(R⁶⁰)C(O)R⁶³, —N(R⁶³)C(O)N(R⁶⁰)₂, —N(R⁶⁰)C(O)N(R⁶⁰)(R⁶³), —N(R⁶⁰)(R⁶³), —C(O)R⁶³, —C(O)OR⁶³, —OC(O)R⁶³, =N(R⁶³), C₃₋₁₂ carbocycle substituted with R⁶³, and 3- to 12-membered heterocycle substituted with R⁶³;

each R⁶³ is independently selected at each occurrence from —C₁₋₁₀ alkylene- attached to -L⁴-RL-COMP, —C₂₋₁₀ alkenylene- attached to -L⁴-RL-COMP, —C₂₋₁₀ alkynylene- attached to -L⁴-RL-COMP, C₃₋₁₂ carbocyclene attached to -L⁴-RL-COMP, and 3- to 12-membered heterocyclene attached to -L⁴-RL-COMP;

L⁴ is a bond or a linker;

RL is a reactive linker residue;

x is an integer between 1 and 30, inclusive;

COMP is a macromolecule; and

L¹, R^{1a}, R^{2a}, R^{1b}, R^{2b}, and R²⁰ are as defined herein.

In one embodiment, the compound conjugate is a compound of Formula (CONJ-III):

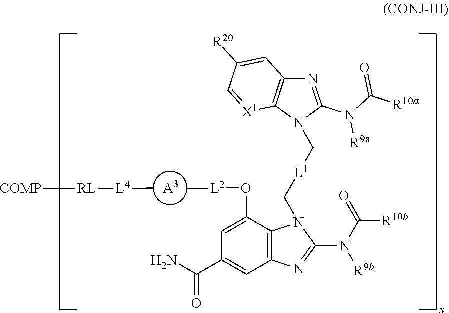

or a pharmaceutically acceptable salt or tautomer thereof;
wherein

L⁴ is a bond or a linker;

RL is a reactive linker residue;

x is an integer between 1 and 30, inclusive;

COMP is a macromolecule; and

Ring A³, L¹, L², X¹, R^{9a}, R^{10a}, R^{9b}, R^{10b}, and R²⁰ are as defined herein.

In one embodiment, the compound conjugate is a compound of Formula (CONJ-IV):

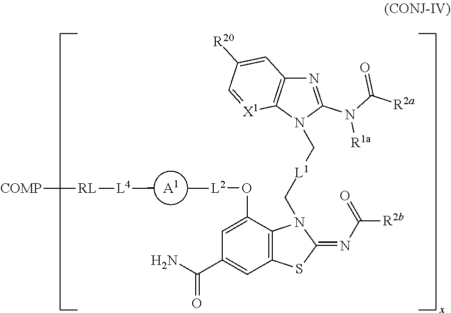

or pharmaceutically acceptable salt or tautomer thereof,

wherein

L⁴ is a bond or a linker;

RL is a reactive linker residue;

x is an integer between 1 and 30, inclusive;

COMP is a macromolecule; and

Ring A¹, L¹, L², X¹, R^{1a}, R^{2a}, R^{2b}, and R²⁰ are as defined herein.

In one embodiment, the compound conjugate is a compound of Formula (CONJ-V):

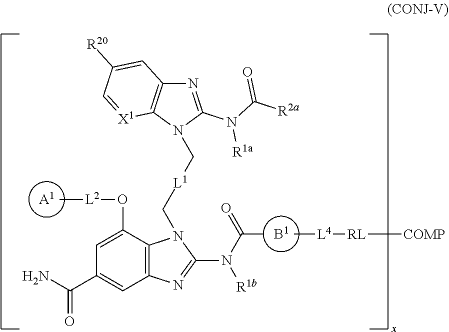

or a pharmaceutically acceptable salt or tautomer thereof,

wherein

L⁴ is a bond or a linker;

RL is a reactive linker residue;

x is an integer between 1 and 30, inclusive;

COMP is a macromolecule; and

Ring A¹, Ring B¹, L¹, L², X¹, R^{1a}, R^{2a}, R^{1b}, and R²⁰ are as defined herein.

The present disclosure provides at least the following embodiments:
a) A compound of Formula (I), Formula (II), or Formula (III) or a pharmaceutically acceptable salt or tautomer thereof;
b) A compound selected from the compounds of Table A or Table A-1 or a pharmaceutically acceptable salt or tautomer thereof;
c) A conjugate compound of Formula (CONJ-I), (CONJ-II), or (CONJ-III) or a pharmaceutically acceptable salt or tautomer thereof;
d) A conjugate compound selected from the conjugate compounds of Table B or Table B-1 or a pharmaceutically acceptable salt or tautomer thereof;
e) A pharmaceutical composition comprising a compound of any one of (a)-(d) and a pharmaceutically acceptable excipient, diluent, or carrier;
f) A linker-payload compound of Formula (LP-I), (LP-II), or (LP-III) or a pharmaceutically acceptable salt or tautomer thereof;
g) A linker-payload selected from the compounds of Table C or Table C-1 or a pharmaceutically acceptable salt or tautomer thereof;
h) A method of treating a disease or disorder mediated by STING in a subject in need thereof comprising administering a therapeutically effective amount of a compound of (a) or (b), a conjugate compound of (c) or (d), or a pharmaceutical composition of (e);
i) The method of (h) wherein the disease or disorder is a cellular proliferative disorder, including, but not limited to cancer;
j) The method of (i) wherein the cancer is selected from acute myeloid leukemia, breast cancer, colorectal cancer, glioma, head and neck squamous cell carcinoma, lung cancer, including non-small cell lung cancer, head and neck cancer, lymphoma, including a malignant lymphoma, melanoma, nasopharyngeal carcinoma, ovary cancer, pancreatic cancer, prostate cancer, urothelial cancer, and tongue squamous cell carcinoma;
k) A method to induce an immune response in a subject in need thereof comprising administering a therapeutically effective amount of a compound of (a) or (b), a conjugate compound of (c) or (d), or a pharmaceutical composition of (e);
l) A method to induce STING-dependent type I interferon production in a subject in need thereof comprising administering a therapeutically effective amount of a compound of (a) or (b), a conjugate compound of (c) or (d), or a pharmaceutical composition of (e);
m) A method to induce STING-dependent cytokine production in a subject in need thereof comprising administering a therapeutically effective amount of a compound of (a) or (b), a conjugate compound of (c) or (d), or a pharmaceutical composition of (e);
n) The method of any one of embodiments (h)-(m) wherein the compound of (a) or (b), the conjugate compound of (c) or (d), or the pharmaceutical composition of (e) is administered in combination with an immune modulator, including but not limited to a checkpoint inhibitor, for example, a PD-1 inhibitor or a CTLA-4 inhibitor;
o) Use of a therapeutically effective amount of a compound of (a) or (b), a conjugate compound of (c) or (d), or a pharmaceutical composition of (e) for the treatment of a disease or disorder mediated by STING in a subject in need thereof;
p) Use of a therapeutically effective amount of a compound of (a) or (b), a conjugate compound of (c) or (d), or a pharmaceutical composition of (e) to induce an immune response in a subject in need thereof;
q) Use of a therapeutically effective amount of a compound of (a) or (b), a conjugate compound of (c) or (d), or a pharmaceutical composition of (e) to induce STING-dependent type I interferon production in a subject in need thereof,
r) Use of a therapeutically effective amount of a compound of (a) or (b), a conjugate compound of (c) or (d), or a pharmaceutical composition of (e) to induce STING-dependent cytokine production in a subject in need thereof,
s) Use of a therapeutically effective amount of a compound of (a) or (b), a conjugate compound of (c) or (d), or a pharmaceutical composition of (e) in the manufacture of a medicament for the treatment of a disease or disorder mediated by STING in a subject in need thereof,
t) Use of a therapeutically effective amount of a compound of (a) or (b), a conjugate compound of (c) or (d), or a pharmaceutical composition of (e) in the manufacture of a medicament to induce an immune response in a subject in need thereof;
u) Use of a therapeutically effective amount of a compound of (a) or (b), a conjugate compound of (c) or (d), or a pharmaceutical composition of (e) in the manufacture of a medicament to induce STING-dependent type I interferon production in a subject in need thereof,
v) Use of a therapeutically effective amount of a compound of (a) or (b), a conjugate compound of (c) or (d), or a pharmaceutical composition of (e) in the manufacture of a medicament to induce STING-dependent cytokine production in a subject in need thereof, and
w) A kit comprising a therapeutically effective amount of a compound of (a) or (b), a conjugate compound of (c) or (d), or a pharmaceutical composition of (e) and instructions for use of the compound.

### BRIEF DESCRIPTION OF THE FIGURES

FIG. 1A-FIG. 1C provide effects of TROP2 conjugates with and without STING agonist on growth of MC38-hTrop2 tumors. FIG. 1A provides tumor volume vs. days post treatment of vehicle, ADC4, or iSAC1. FIG. 1B provides tumor volume vs. days post treatment of vehicle, ADC4, or iSAC14. FIG. 1C provides tumor volume vs. days post treatment of vehicle, ADC6, or iSAC2.

### DETAILED DESCRIPTION

### I. Definitions

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, the term “about” indicates the designated value ±one standard deviation of that value. In certain embodiments, for example, logarithmic scales (e.g., pH), the term “about” indicates the designated value ±0.3, ±0.2, or ±0.1.

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

“Alkoxy” and “alkoxyl,” refer to the group —OR″ where R″ is alkyl or cycloalkyl. Alkoxy groups include, in certain embodiments, methoxy, ethoxy, n-propoxy, isopropoxy, n-butoxy, tert-butoxy, sec-butoxy, n-pentoxy, n-hexoxy, 1,2-dimethylbutoxy, and the like.

The term “alkoxyamine,” as used herein, refers to the group -alkylene-O—NH₂, wherein alkylene is as defined herein. In some embodiments, alkoxyamine groups can react with aldehydes to form oxime residues. Examples of alkoxyamine groups include —CH₂CH₂—O—NH₂, —CH₂—O—NH₂, and —O—NH₂.

The term “alkyl,” as used herein, unless otherwise specified, refers to a saturated straight or branched hydrocarbon. In certain embodiments, the alkyl group is a primary, secondary, or tertiary hydrocarbon. In certain embodiments, the alkyl group includes one to ten carbon atoms (i.e., C₁ to C₁₀ alkyl). In certain embodiments, the alkyl is a lower alkyl, for example, C₁₋₆alkyl, and the like. In certain embodiments, the alkyl group is selected from the group consisting of methyl, ethyl, propyl, isopropyl, butyl, isobutyl, sec-butyl, t-butyl, pentyl, isopentyl, neopentyl, hexyl, isohexyl, 3-methylpentyl, 2,2-dimethylbutyl, and 2,3-dimethylbutyl. In certain embodiments, “substituted alkyl” refers to an alkyl substituted with, for example, one, two, or three groups independently selected from a halogen (e.g., fluoro (F), chloro (Cl), bromo (Br), or iodo (I)), alkyl, —CN, —NO₂, amido, —C(O)—, —C(S)—, ester, carbamate, alkenyl, alkynyl, cycloalkyl, heterocycloalkyl, dialkylamino, haloalkyl, hydroxyl, amino, alkylamino, and alkoxy. In some embodiments, alkyl is unsubstituted.

The term “alkylene,” as used herein, unless otherwise specified, refers to a divalent alkyl group, as defined herein. “Substituted alkylene” refers to an alkylene group substituted as described herein for alkyl. In some embodiments, alkylene is unsubstituted.

“Alkenyl” refers to an olefinically unsaturated hydrocarbon group, in certain embodiments, having up to about eleven carbon atoms or from two to six carbon atoms (e.g., “lower alkenyl”), which can be straight-chained or branched, and having at least one or from one to two sites of olefinic unsaturation. “Substituted alkenyl” refers to an alkenyl group substituted as described herein for alkyl.

“Alkenylene” refers to a divalent alkenyl as defined herein. Lower alkenylene is, for example, C₂-C₆-alkenylene.

“Alkynyl” refers to acetylenically unsaturated hydrocarbon groups, in certain embodiments, having up to about eleven carbon atoms or from two to six carbon atoms (e.g., “lower alkynyl”), which can be straight-chained or branched, and having at least one or from one to two sites of acetylenic unsaturation. Non-limiting examples of alkynyl groups include acetylene (—C≡CH), propargyl (—CH₂C≡CH), and the like. “Substituted alkynyl” refers to an alkynyl group substituted as described herein for alkyl.

“Alkynylene” refers to a divalent alkynyl as defined herein. Lower alkynylene is, for example, C₂-C₆-alkynylene.

“Amino” refers to —NH₂.

The term “aminoalkyl,” as used herein, and unless otherwise specified, refers to an alkyl group, as defined herein, which is substituted with one or more amino groups. In some embodiments, the aminoalkyl is an alkyl group substituted with one —NH₂ group (e.g., —R′(NH₂) wherein R′ is alkyl as defined herein).

The term “alkylamino,” as used herein, and unless otherwise specified, refers to the group —NHR″ where R″ is, for example, C₁₋₁₀alkyl, C₂₋₁₀ alkenyl, C₂₋₁₀ alkynyl, C₃₋₁₂ carbocycle, 3- to 12-membered heterocycle, C₁₋₁₀ haloalkyl, and the like as defined herein. In certain embodiments, alkylamino is C₁₋₆alkylamino.

The term “dialkylamino,” as used herein, and unless otherwise specified, refers to the group —NR″R″ where each R″ is independently C₁₋₁₀alkyl, as defined herein. In certain embodiments, dialkylamino is, for example, di-C₁₋₆alkylamino, C₂₋₁₀ alkenyl, C₂₋₁₀ alkynyl, C₃₋₁₂ carbocycle, 3- to 12-membered heterocycle, C₁₋₁₀ haloalkyl, and the like.

The term “aryl,” as used herein, and unless otherwise specified, refers to phenyl, biphenyl, or naphthyl. The term includes both substituted and unsubstituted moieties. An aryl group can be substituted with any described moiety including, but not limited to, one or more moieties (e.g., in some embodiments one, two, or three moieties) selected from the group consisting of halogen (e.g., fluoro (F), chloro (Cl), bromo (Br), or iodo (I)), alkyl, haloalkyl, hydroxyl, amino, alkylamino, arylamino, alkoxy, aryloxy, nitro, cyano, sulfonic acid, sulfate, phosphonic acid, phosphate, and phosphonate, wherein each moiety is independently either unprotected, or protected as necessary, as would be appreciated by those skilled in the art (see, e.g., Greene, et al., Protective Groups in Organic Synthesis, John Wiley and Sons, Second Edition, 1991); and wherein the aryl in the arylamino and aryloxy substituents are not further substituted.

The term “arylamino,” as used herein, and unless otherwise specified, refers to an —NR′R″ group where R′ is hydrogen or C₁-C₆-alkyl; and R″ is aryl, as defined herein.

The term “arylene,” as used herein, and unless otherwise specified, refers to a divalent aryl group, as defined herein.

The term “aryloxy,” as used herein, and unless otherwise specified, refers to an —OR group where R is aryl, as defined herein.

“Alkarylene” refers to an arylene group, as defined herein, wherein the aryl ring is substituted with one or two alkyl groups. “Substituted alkarylene” refers to an alkarylene, as defined herein, where the arylene group is further substituted, as defined herein for aryl.

“Aralkylene” refers to a —CH₂-arylene-, -arylene-CH₂—, or —CH₂-arylene-CH₂— group, where arylene is as defined herein. “Substituted aralkylene” refers to an aralkylene, as defined herein, where the aralkylene group is substituted, as defined herein for aryl.

“Carboxyl” or “carboxy” refers to —C(O)OH or —COOH.

The term “carbocycle” as used herein, unless otherwise specified, refers to a saturated, unsaturated, or aromatic ring in which all atoms of the ring are carbon. In certain embodiments, the “carbocycle” group may be saturated, and/or bridged, and/or non-bridged, and/or a fused bicyclic group, and/or a spirocyclic bicyclic group. In certain embodiments, the “carbocycle” group includes three to ten carbon atoms (i.e., C₃ to C₁₀ cycloalkyl). In some embodiments, the “carbocycle” has from three to fifteen carbons (C₃₋₁₅), from three to ten carbons (C₃₋₁₀), from three to seven carbons (C₃₋₇), or from three to six carbons (C₃-C₆) (i.e., “lower cycloalkyl”). In certain embodiments, the “carbocycle” group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl. Exemplary “carbocycles” include cyclopentyl, cyclohexyl, cyclohexenyl, adamantyl, phenyl, indanyl, and naphthyl. “Carbocycle” includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Non-limiting examples of bridged bicyclic carbocycle groups include, but are not limited to, bicyclo[1.1.1]pentyl, bicyclo[2.1.1]hexyl, bicyclo[2.1.1]hexyl, bicyclo[3.1.1]heptyl, bicyclo[2.2.1]heptyl, bicyclo[3.2.1]octyl, bicyclo[2.2.2]octyl, bicyclo[3.3.1]nonyl, bicyclo[3.3.2]decyl, and 2-oxabicyclo[2.2.2]octyl. Non-limiting examples of spirocyclic carbocycle groups include, but are not limited to, spiro[3.3]heptyl, spiro[3.4]octyl, spiro[3.5]nonyl, spiro[3.6]decyl, spiro[4.4]nonyl, spiro[4.5]decyl, spiro[5.5]undecyl, spiro[5.6]dodecyl, and spiro[5.7]tridecyl.

“Carbocyclene” refers to a divalent carbocycle as defined herein.

The term “bicyclic ring system” includes 6-12 (e.g., 8-12 or 9-, 10-, or 11-) membered structures that form two rings, wherein the two rings have at least one atom in common (e.g., two atoms in common). Bicyclic rings can be fused, bridged, or spirocyclic. Bicyclic ring systems include bicycloaliphatics (e.g., bicycloalkyl or bicycloalkenyl), bicycloheteroaliphatics, bicyclic aryls, and bicyclic heteroaryls.

The term “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[1.1.1]pentyl, bicyclo[2.1.1]hexyl, bicyclo[2.1.1]hexyl, bicyclo[3.1.1]heptyl, bicyclo[2.2.1]heptyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 6-azabicyclo[3.1.1]heptyl, 6-azabicyclo[3.1.1]heptyl, 1-azabicyclo[2.2.1]heptyl, 2-azabicyclo[2.2.1]heptyl, 7-azabicyclo[2.2.1]heptyl, 1-azabicyclo[2.2.2]octyl, 3-azabicyclo[3.2.1]octyl, and 2-oxabicyclo[3.1.1]heptyl, 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

The term “spiro bicyclic ring system” refers to a bicyclic heterocyclicalipahtic ring system or bicyclic cycloaliphatic ring system in which 2 or 3 rings are linked together by one common atom. Spiro compounds depicted with overlapping rings indicate that the rings can bond at any vertex. For instance, in the spiro group

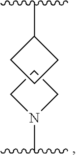

the two rings can bond at any of the three available vertex atoms in either ring. In some embodiments, a spiro bicyclic ring is a 3-to 12-membered spirocyclic bicyclic heterocycle comprising two nitrogen atoms and one oxygen atom. Non-limiting examples of a spirocyclic bicyclic heterocycle include a 10-membered spirocyclic bicyclic heterocycle, a 9-membered spirocyclic bicyclic heterocycle, and a 8-membered spirocyclic bicyclic heterocycle. The 3- to 12-membered spirocyclic bicyclic heterocycle include, but are not limited to, nitrogen (N), oxygen (O), and sulfur (S) atoms, for example two nitrogen atoms and one oxygen atom. For example, a 5-oxa-2,8-diazaspiro[3.5]nonane is a compound in which a 4 membered heterocyclic ring and a 6 membered heterocyclic ring are bonded through a single carbon atom wherein an oxygen atom is in the 6 membered heterocyclo ring.

The term “cycloalkylene,” as used herein refers to a divalent cycloalkyl group, as defined herein. In certain embodiments, the cycloalkylene group is cyclopropylene

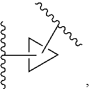

cyclobutylene

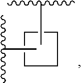

cyclopentylene

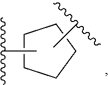

cyclohexylene

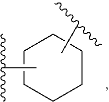

cycloheptylene

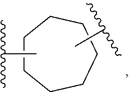

and the like. Lower cycloalkylene refers to a C₃-C₆-cycloalkylene.

The term “cycloalkylalkyl,” as used herein, unless otherwise specified, refers to an alkyl group, as defined herein, substituted with one or two cycloalkyl, as defined herein.

The term “ester,” as used herein, refers to —C(O)OR or —COOR where R is alkyl, as defined herein.

The term “fluorene” as used herein refers to

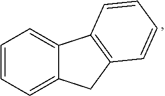 [SMILES: c1ccc2c(c1)Cc1ccccc1-2]

wherein any one or more carbons bearing one or more hydrogens can be substituted with a chemical functional group as described herein.

The term “haloalkyl” refers to an alkyl group, as defined herein, substituted with one or more halogen atoms (e.g., in some embodiments one, two, three, four, or five) which are independently selected.

The term “heteroalkyl” refers to an alkyl, as defined herein, in which one or more carbon atoms are replaced by heteroatoms. As used herein, “heteroalkenyl” refers to an alkenyl, as defined herein, in which one or more carbon atoms are replaced by heteroatoms. As used herein, “heteroalkynyl” refers to an alkynyl, as defined herein, in which one or more carbon atoms are replaced by heteroatoms. Suitable heteroatoms include, but are not limited to, nitrogen (N), oxygen (O), and sulfur (S) atoms. Heteroalkyl, heteroalkenyl, and heteroalkynyl are optionally substituted. Examples of heteroalkyl moieties include, but are not limited to, aminoalkyl, sulfonylalkyl, and sulfinylalkyl. Examples of heteroalkyl moieties also include, but are not limited to, methylamino, methylsulfonyl, and methylsulfinyl. “Substituted heteroalkyl” refers to heteroalkyl substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heteroalkyl group may comprise one, two, three, or four heteroatoms. Those of skill in the art will recognize that a 4-membered heteroalkyl may generally comprise one or two heteroatoms, a 5- or 6-membered heteroalkyl may generally comprise one, two, or three heteroatoms, and a 7- to 10-membered heteroalkyl may generally comprise one, two, three, or four heteroatoms.

The term “heteroalkylene,” as used herein, refers to a divalent heteroalkyl, as defined herein. “Substituted heteroalkylene” refers to a divalent heteroalkyl, as defined herein, substituted as described for heteroalkyl.

The term “heterocycle” refers to a saturated, unsaturated or aromatic ring comprising one or more heteroatoms. Exemplary heteroatoms include N, O, Si, P, B, and S atoms where the nitrogen or sulfur atoms may be optionally oxidized, and the nitrogen atoms may be optionally quaternized and the remaining ring atoms of the non-aromatic ring are carbon atoms. A “heterocycle” includes 3- to 10-membered monocyclic rings, 6- to 12-membered bicyclic rings, and 6- to 12-membered bridged rings. In certain embodiments, “heterocycle” is a monovalent, monocyclic, or multicyclic fully-saturated ring system. In certain embodiments, the “heterocycle” group may be unsaturated, and/or bridged, and/or non-bridged, and/or a fused bicyclic group, and/or a spirocyclic bicyclic group. A bicyclic “heterocycle” includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. In certain embodiments, the “heterocycle” group has from three to twenty, from three to fifteen, from three to ten, from three to eight, from four to seven, from four to eleven, or from five to six ring atoms. The “heterocycle” may be attached to a core structure at any heteroatom or carbon atom which results in the creation of a stable compound. In certain embodiments, the “heterocycle” is a monocyclic, bicyclic, tricyclic, or tetracyclic ring system, which may include a fused or bridged or spirocyclic ring system and in which the nitrogen or sulfur atoms may be optionally oxidized, and/or the nitrogen atoms may be optionally quaternized. In some embodiments, “heterocycle” radicals include, but are not limited to, 2,5-diazabicyclo[2.2.2]octanyl, decahydroisoquinolinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. Non-limiting examples of bridged heterocycle groups include, but are not limited to, 6-azabicyclo[3.1.1]heptyl, 6-azabicyclo[3.1.1]heptyl, 1-azabicyclo[2.2.1]heptyl, 2-azabicyclo[2.2.1]heptyl, 7-azabicyclo[2.2.1]heptyl, 1-azabicyclo[2.2.2]octyl, 3-azabicyclo[3.2.1]octyl, and 2-oxabicyclo[3.1.1]heptyl, 2,6-dioxa-tricyclo[3.3.1.0^{3,7}]nonyl. Non-limiting examples of spirocyclic heterocycle groups include, but are not limited to, 2,8-diazaspiro[4.5]decyl; 2,7-diazaspiro[3.5]nonyl; 3,9-diazaspiro[5.5]undecyl; 3-azaspiro[5.5]undecyl; 2-oxa-6-azaspiro[3.4]octyl; 2-oxa-9-azaspiro[5.5]undecyl; 3-oxa-9-azaspiro[5.5]undecyl; 7-azaspiro[3.5]nonyl; 2-azaspiro[3.5]nonyl; 7-oxaspiro[3.5]nonyl; and 2-oxaspiro[3.5]nonyl. In certain embodiments, “heterocycle” may also be optionally substituted as described herein. In certain embodiments, “heterocycle” is substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a “heterocycle” group may comprise one, two, three, or four heteroatoms. Those of skill in the art will recognize that a 4-membered “heterocycle” may generally comprise one or two heteroatoms, a 5- or 6-membered “heterocycle” may generally comprise one, two, or three heteroatoms, and a 7- to 10-membered “heterocycle” may generally comprise one, two, three, or four heteroatoms.

“Heterocycloalkylene” refers to a divalent heterocycloalkyl as defined herein.

The term “heteroaryl” refers to a monovalent, monocyclic aromatic group and/or multicyclic aromatic group, wherein at least one aromatic ring contains one or more heteroatoms independently selected from oxygen, sulfur, and nitrogen within the ring. Each ring of a heteroaryl group can contain one or two oxygen atoms, one or two sulfur atoms, and/or one to four nitrogen atoms, provided that the total number of heteroatoms in each ring is four or less and each ring contains at least one carbon atom. In certain embodiments, the heteroaryl has from five to twenty, from five to fifteen, or from five to ten ring atoms. A heteroaryl may be attached to the rest of the molecule via a nitrogen or a carbon atom. In some embodiments, monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, triazolyl, thiadiazolyl, thiazolyl, thienyl, tetrazolyl, and triazinyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein. “Substituted heteroaryl” is a heteroaryl substituted as defined for aryl.

The term “heteroarylene” refers to a divalent heteroaryl group, as defined herein. “Substituted heteroarylene” is a heteroarylene substituted as defined for aryl.

The term “protecting group,” as used herein, and unless otherwise specified, refers to a group that is added to an oxygen, nitrogen, or phosphorus atom to prevent further reaction at the (protected) oxygen, nitrogen, or phosphorus, or for other purposes. A wide variety of oxygen and nitrogen protecting groups are known to those skilled in the art of organic synthesis (see, e.g., Greene, et al., Protective Groups in Organic Synthesis, John Wiley and Sons, Fourth Edition, 2006, which is incorporated herein by reference in its entirety).

“Pharmaceutically acceptable salt” refers to any salt of a compound provided herein which retains its biological properties and which is not toxic or otherwise undesirable for pharmaceutical use. Such salts may be derived from a variety of organic and inorganic counter-ions well known in the art. Such salts include, but are not limited to (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, glutamic, hydroxynaphthoic, salicylic, stearic, cyclohexylsulfamic, quinic, and muconic acids, and the like; or (2) salts formed when an acidic proton present in the parent compound either (a) is replaced by a metal ion, for example, an alkali metal ion, an alkaline earth ion, or an aluminum ion, or alkali metal or alkaline earth metal hydroxides, such as sodium, potassium, calcium, magnesium, aluminum, lithium, zinc, and barium hydroxide, or ammonia; or (b) coordinates with an organic base, such as aliphatic, alicyclic, or aromatic organic amines, including, without limitation, ammonia, methylamine, dimethylamine, diethylamine, picoline, ethanolamine, diethanolamine, triethanolamine, ethylenediamine, lysine, arginine, ornithine, choline, N,N-dibenzylethylene-diamine, chloroprocaine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example and without limitation, sodium, potassium, calcium, magnesium, ammonium, and tetraalkylammonium salts, and the like, and when the compound contains a basic functionality, salts of non-toxic organic or inorganic acids, such as hydrohalides, for example, hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, glutamate, hydroxynaphthoate, salicylate, stearate, cyclohexylsulfamate, quinate, muconate, and the like.

The term “substantially free of” or “substantially in the absence of” with respect to a composition refers to a composition that includes at least 85% or 90% by weight, in certain embodiments 95%, 98%, 99%, or 100% by weight; or in certain embodiments, 95%, 98%, 99%, or 100% of the designated enantiomer or diastereomer of a compound. In certain embodiments, in the methods and compounds provided herein, the compounds are substantially free of one of two enantiomers. In certain embodiments, in the methods and compounds provided herein, the compounds are substantially free of one of two diastereomers. In certain embodiments, in the methods and compounds provided herein, the compounds are substantially free of enantiomers (i.e., the compounds are not a racemic or 50:50 mixture of compounds).

Similarly, the term “isolated” with respect to a composition refers to a composition that includes at least 85%, 90%, 95%, 98%, or 99% to 100% by weight, of the compound, the remainder comprising other chemical species, enantiomers, or diastereomers.

“Solvate” refers to a compound provided herein, or a salt thereof, that further includes a stoichiometric or non-stoichiometric amount of solvent bound by non-covalent intermolecular forces. Where the solvent is water, the solvate is a hydrate.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (=O), thioxo (=S), cyano (—CN), nitro (—NO₂), imino (=N—H), oximo (=N—OH), hydrazino (=N— NH₂), —R^{b1}—OR^{a1}, —R^{b1}—OC(O)—R^{a1}, —R^{b1}—OC(O)—OR^{a1}, —R^{b1}—OC(O)—N(R^{a1})₂, —R^{b1}—N(R^{a1})₂, —R^{b1}—C(O)R^{a1}, —R^{b1}—C(O)OR^{a1}, —R^{b1}—C(O)N(R^{a1})₂, —R^{b1}—O—R^{c1}—C(O)N(R^{a1})₂, —R^{b1}—N(R^{a1})C(O)OR^{a1}, —R^{b1}—N(R^{a1})C(O)R^{a1}, —R^{b1}—N(R^{a1})S(O)R^{a1} (where t is 1 or 2), —R^{b1}—S(O)ₜR^{a1} (where t is 1 or 2), —R^{b1}—S(O)ₜOR^{a1} (where t is 1 or 2), and —R^{b1}—S(O)ₜN(R^{a1})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (=O), thioxo (=S), cyano (—CN), nitro (—NO₂), imino (=N—H), oximo (=N—OH), hydrazine (=N— NH₂), —R^{b1}—OR^{a1}, —R^{b1}OC(O)—R^{a1}, R^{b1}—OC(O)—OR^{a1}, —R^{b1}—OC(O)—N(R^{a1})₂, —R^{b1}—N(R^{a1})₂, —R^{b1}—C(O)R^{a1}, —R^{b1}—C(O)OR^{a1}, —R^{b1}—C(O)N(R^{a1})₂, —R^{b1}—O—R^{c1}—C(O)N(R^{a1})₂, —R^{b1}—N(R^{a1})C(O)OR^{a1}, —R^{b1}—N(R^{a1})C(O)R^{a1}, —R^{b1}—N(R^{a1})S(O)ₜR^{a1} (where t is 1 or 2), —R^{b1}—S(O)ₜR^{a1} (where t is 1 or 2), —R^{b1}—S(O) OR^{a1} (where t is 1 or 2) and —R^{b1}—S(O)ᵣN(R^{a1})₂ (where t is 1 or 2); wherein each R^{a1} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a1}, valence permitting, may be optionally substituted with alkyl, alkenyl, alkynyl, halogen, haloalkyl, haloalkenyl, haloalkynyl, oxo (=O), thioxo (=S), cyano (—CN), nitro (—NO₂), imino (=N—H), oximo (=N—OH), hydrazine (=N—NH₂), —R^{b1}—OR^{a1}, —R^{b1}—OC(O)—R^{a1}, —R^{b1}—OC(O)—OR^{a1}, —R^{b1}—OC(O)—N(R^{a1})₂, —R^{b1}—N(R^{a1})₂, —R^{b1}—C(O)R^{a1}, —R^{b1}—C(O)OR^{a1}, —R^{b1}—C(O)N(R^{a1})₂, —R^{b1}—O—R^{c1}—C(O)N(R^{a1})₂, —R^{b1}—N(R^{a1})C(O)OR^{a1}, —R^{b1}—N(R^{a1})C(O)R^{a1}, —R^{b1}—N(R^{a1})S(O)R^{a1} (where t is 1 or 2), —R^{b1}—S(O)ₜR^{a1} (where t is 1 or 2), —R^{b1}—S(O)ₜOR^{a1} (where t is 1 or 2) and —R^{b1}—S(O)ₜN(R^{a1})₂ (where t is 1 or 2); and wherein each R^{b1} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate.

“Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as hydrogen (H), the position is understood to have hydrogen at its natural isotopic composition.

“Isotopic enrichment” refers to the percentage of incorporation of an amount of a specific isotope at a given atom in a molecule in the place of that atom's natural isotopic abundance. For example, deuterium (D) enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “alkyl,” “alkylene,” “alkylamino,” “dialkylamino,” “cycloalkyl,” “aryl,” “arylene,” “alkoxy,” “amino,” “carboxyl,” “heterocycloalkyl,” “heteroaryl,” “heteroarylene,” “carboxyl,” and “amino acid” groups optionally comprise deuterium (D) at one or more positions where hydrogen (H) atoms are present, and wherein the deuterium composition of the atom or atoms is other than the natural isotopic composition.

Also as used herein, “alkyl,” “alkylene,” “alkylamino,” “dialkylamino,” “cycloalkyl,” “aryl,” “arylene,” “alkoxy,” “amino,” “carboxyl,” “heterocycloalkyl,” “heteroaryl,” “heteroarylene,” “carboxyl,” and “amino acid” groups optionally comprise carbon-13 (¹³C) at an amount other than the natural isotopic composition.

The term “macromolecule” or “macromolecular moiety” refers to a protein, peptide, antibody, nucleic acid, carbohydrate, or other large molecule composed of polymerized monomers. They include peptides of two or more residues, or ten or more residues. In certain embodiments, a macromolecule is at least 1000 Da in mass. In certain embodiments, a macromolecule has at least 1000 atoms. In certain embodiments, a macromolecule can be modified. For instance, a protein, peptide, or antibody can be modified with one or more carbohydrates and/or small molecule therapeutic compounds.

The term “immunoglobulin” refers to a class of structurally related proteins generally comprising two pairs of polypeptide chains: one pair of light (L) chains, and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H} or VH) and a heavy chain constant region (C_{H} or CH). The heavy chain constant region typically comprises three domains, abbreviated C_{H}1 (or CH1), C_{H}2 (or CH2), and C_{H}3 (or CH3). Each light chain typically comprises a light chain variable region (V_{L} or VL) and a light chain constant region. The light chain constant region typically comprises one domain, abbreviated C_{L} or CL.

The term “antibody” is used herein in its broadest sense. An antibody includes intact antibodies (e.g., intact immunoglobulins), and antibody fragments (e.g., antigen binding fragments or antigen-binding fragments of antibodies). Antibodies comprise at least one antigen-binding domain. One example of an antigen-binding domain is an antigen binding domain formed by a V_{H}-V_{L} dimer.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments. “Fv” fragments comprise a non-covalently-linked dimer of one heavy chain variable domain and one light chain variable domain. “Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody. “F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol. “Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). Antibodies from Escherichia coli. In Rosenberg M. & Moore G. P. (Eds.), The Pharmacology of Monoclonal Antibodies vol. 113 (pp. 269-315). Springer-Verlag, New York, are incorporated by reference in their entirety. “scFv-Fc” fragments comprise an scFv attached to an Fc domain. For example, an Fc domain may be attached to the C-terminus of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG1 Fc domain.

The term “amino acid” or “amino acid residue” refers to a D- or L-natural or non-naturally occurring amino acid. refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V), and the less common cystine, pyrrolysine, and selenocysteine. Unnatural amino acids are not proteinogenic amino acids, or post-translationally modified variants thereof. In particular, the term unnatural amino acid refers to an amino acid that is not one of the twenty common amino acids, cystine, pyrrolysine, or selenocysteine, or post-translationally modified variants thereof. Non-limiting examples of unnatural amino acids include sulfoalanine, hydroxyproline (Hyp), beta-alanine, citrulline (Cit), ornithine (Orn), norleucine (Nle), 3-nitrotyrosine, nitroarginine, pyroglutamic acid (Pyr), naphtylalanine (Nal), 2,4-diaminobutyric acid (DAB), methionine sulfoxide, and methionine sulfone. Naturally encoded amino acids include post-translation modification (PTM) or post-translational variants of the twenty-two naturally occurring amino acids such as prenylated amino acids, isoprenylated amino acids, myrisoylated amino acids, palmitoylated amino acids, N-linked glycosylated amino acids, O-linked glycosylated amino acids, phosphorylated amino acids, and acylated amino acids. The term “amino acid” also includes non-natural (or unnatural) or synthetic α-, β-, γ-, or δ-amino acids, and includes, but is not limited to, amino acids found in proteins, i.e., glycine, alanine, valine, leucine, isoleucine, methionine, phenylalanine, tryptophan, proline, serine, threonine, cysteine, tyrosine, asparagine, glutamine, aspartate, glutamate, lysine, arginine, and histidine. In certain embodiments, the amino acid is in the L-configuration. In certain embodiments, the amino acid is in the D-configuration. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl, or β-histidinyl. In certain embodiments, the amino acid is alkylated. In certain embodiments, the amino acid is methylated. In certain embodiments, the amino acid is N-methylated. In certain embodiments, the amino acid is O-methylated.

The term “conjugate” refers to a compound or drug moiety described herein linked to one or more macromolecular moieties. The macromolecular moiety is as defined herein or is any macromolecule deemed suitable to the person of skill in the art. The compound or drug moiety can be any compound or drug moiety described herein. The compound or drug moiety can be directly linked to the macromolecular moiety via a covalent bond, or the compound or drug moiety can be linked to the macromolecular moiety indirectly via a linker. Typically, the linker is covalently bonded to the macromolecular moiety and also covalently bonded to the compound or drug moiety.

“pAMF,” “pAMF residue,” or “pAMF mutation” refers to a variant phenylalanine residue (i.e., para-azidomethyl-L-phenylalanine) added or substituted into a polypeptide.

The term “linker” refers to a molecular moiety that is capable of forming at least two covalent bonds. Typically, a linker is capable of forming at least one covalent bond to a macromolecular moiety and at least another covalent bond to a compound. In certain embodiments, a linker can form more than one covalent bond to a macromolecular moiety. In certain embodiments, a linker can form more than one covalent bond to a compound or can form covalent bonds to more than one compound. After a linker forms a bond to a macromolecular moiety, or a compound or both, the remaining structure (i.e. the residue of the linker (“linker residue”) after one or more covalent bonds are formed) may still be referred to as a “linker” herein. The term “linker precursor” refers to a linker having one or more reactive groups capable of forming a covalent bond with a macromolecule, or compound, or both. A person of ordinary skill in the art, given the context of how the term linker is used, would understand whether “linker” means linker precursor with one reactive group, a linker precursor with more than one reactive groups, a linker residue which is covalently bonded to the macromolecule, a linker residue which is covalently bonded to a compound, and/or a linker residue which is covalently bonded to the macromolecule and is covalently bonded to a compound. In some embodiments, the linker is a cleavable linker. For example, a cleavable linker can be one that is released by a bio-labile or enzymatic function, which may or may not be engineered. In some embodiments, the linker is a non-cleavable linker. For example, a non-cleavable linker can be one that is released upon degradation of the macromolecular moiety.

As used herein, term “EC₅₀” refers to a dosage, concentration, or amount of a particular test compound that elicits a dose-dependent response at 50% of maximal expression of a particular response that is induced, provoked, or potentiated by the particular test compound.

As used herein, and unless otherwise specified, the term “IC₅₀” refers to an amount, concentration, or dosage of a particular test compound that achieves a 50% inhibition of a maximal response in an assay that measures such response.

As used herein, the terms “subject” and “patient” are used interchangeably. The terms “subject” and “subjects” refer to an animal, such as a mammal including a non-primate (e.g., a cow, pig, horse, cat, dog, rat, and mouse) and a primate (e.g., a monkey, such as a cynomolgous monkey, a chimpanzee, and a human), and in certain embodiments, a human. In certain embodiments, the subject is a farm animal (e.g., a horse, a cow, a pig, etc.) or a pet (e.g., a dog or a cat). In certain embodiments, the subject is a human.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound or conjugate provided herein. In certain embodiments, a therapeutic agent is an agent which is known to be useful for, or has been or is currently being used for the treatment or prevention of a disorder or one or more symptoms thereof.

“Therapeutically effective amount” refers to an amount of a compound, conjugate, or composition that, when administered to a subject for treating a condition, is sufficient to effect such treatment for the condition. A “therapeutically effective amount” can vary depending on, inter alia, the compound, the conjugate, the disease or disorder and its severity, and the age, weight, etc., of the subject to be treated.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder, or delaying or preventing recurrence of the disease or disorder. In yet another embodiment, “treating” or “treatment” includes the reduction or elimination of either the disease or disorder, or retarding the progression of the disease or disorder or of one or more symptoms of the disease or disorder, or reducing the severity of the disease or disorder or of one or more symptoms of the disease or disorder.

As used herein, the term “inhibits growth” (e.g., referring to cells, such as tumor cells) is intended to include any measurable decrease in cell growth (e.g., tumor cell growth) when contacted with a compound, or conjugate herein, as compared to the growth of the same cells not in contact with the compound, or conjugate herein. In some embodiments, growth may be inhibited by at least about 10%, 20%, 30%, 40%, 50%, 60%, 70%, 80%, 90%, 99%, or 100%. The decrease in cell growth can occur via a variety of mechanisms, including but not limited to, conjugate or compound, internalization, apoptosis, necrosis, and/or effector function-mediated activity.

As used herein, the terms “prophylactic agent” and “prophylactic agents” as used refer to any agent(s) which can be used in the prevention of a disorder or one or more symptoms thereof. In certain embodiments, the term “prophylactic agent” includes a compound, conjugate or composition provided herein. In certain other embodiments, the term “prophylactic agent” does not refer to a compound, conjugate or composition provided herein. For example, a prophylactic agent is an agent which is known to be useful for, or has been or is currently being used to prevent or impede the onset, development, progression, and/or severity of a disorder.

As used herein, the phrase “prophylactically effective amount” refers to the amount of a therapy (e.g., prophylactic agent) which is sufficient to result in the prevention or reduction of the development, recurrence, or onset of one or more symptoms associated with a disorder or to enhance or improve the prophylactic effect(s) of another therapy (e.g., another prophylactic agent).

In some chemical structures illustrated herein, certain substituents, chemical groups, and atoms are depicted with a curvy/wavy/wiggly line

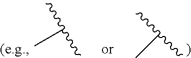

that intersects a bond or bonds to indicate the atom through which the substituents, chemical groups, and atoms are bonded. For example, in some structures, such as but not limited to,

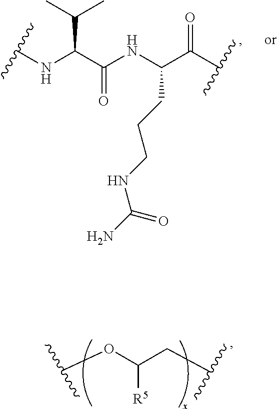

this curvy/wavy/wiggly line indicates the atoms in the backbone of a conjugate, compound, or drug moiety structure to which the illustrated chemical entity is bonded. In some structures, such as but not limited to

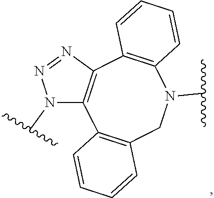 [SMILES: *N1Cc2ccccc2-c2c(nnn2*)-c2ccccc21]

this curvy/wavy/wiggly line indicates the atoms in the macromolecule as well as the atoms in the backbone of a conjugate, compound, or drug moiety structure to which the illustrated chemical entity is bonded.

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains. For example, the group,

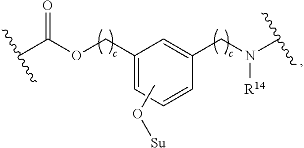

wherein the positions of substituent O-Su are described generically, i.e., not directly attached to any vertex of the bond line structure, i.e., specific ring carbon atom, includes the following, non-limiting examples of groups in which the substituent O-Su is bonded to a specific ring carbon atom:

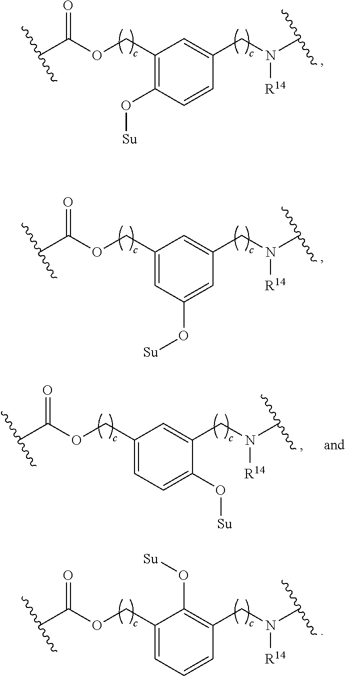

The term “site-specific” refers to a modification of a polypeptide at a predetermined sequence location in the polypeptide. The modification is at a single, predictable residue of the polypeptide with little or no variation. In particular embodiments, a modified amino acid is introduced at that sequence location, for instance recombinantly or synthetically. Similarly, a moiety can be “site-specifically” linked to a residue at a particular sequence location in the polypeptide. In certain embodiments, a polypeptide can comprise more than one site-specific modification.

The term “cancer” is used throughout the specification to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant hematogenous, ascitic and solid tumors.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell proliferation, immune cell differentiation, and cytokine expression.

### II. Compounds, Linker-Payloads, and Conjugates

Provided herein are compounds of Formula (I), (II), (III), and (IV):

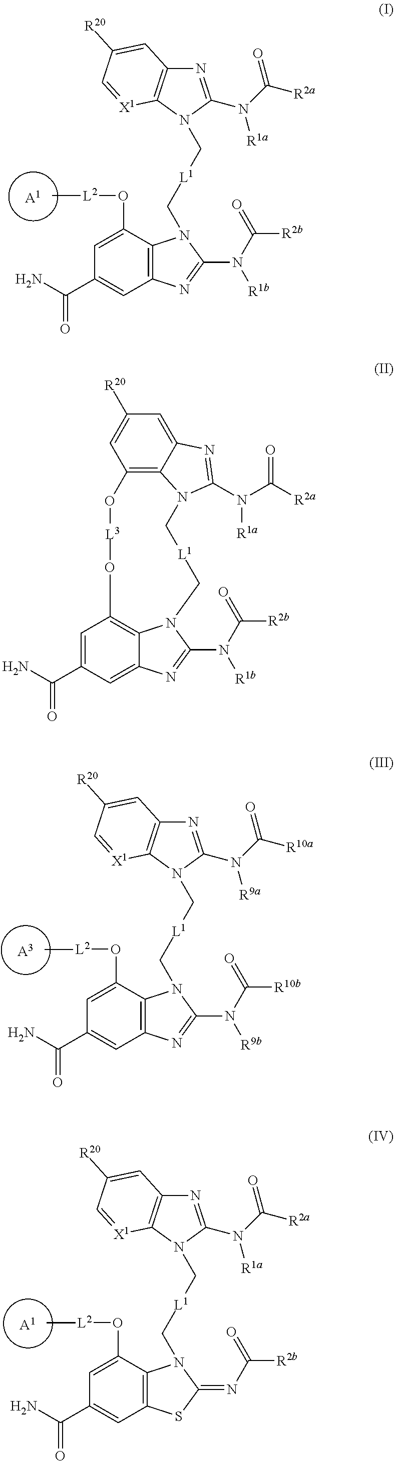

or a pharmaceutically acceptable salt thereof or tautomer thereof,

wherein Ring A¹, Ring A³, X¹, L¹, L², L³, X¹, R^{1a}, R^{1b}, R^{2a}, R^{2b}, and R²⁰ are as defined herein.

Also provided herein are compounds of Formula (LP-I), (LP-II), (LP-III), (LP-IV), and (LP-V):

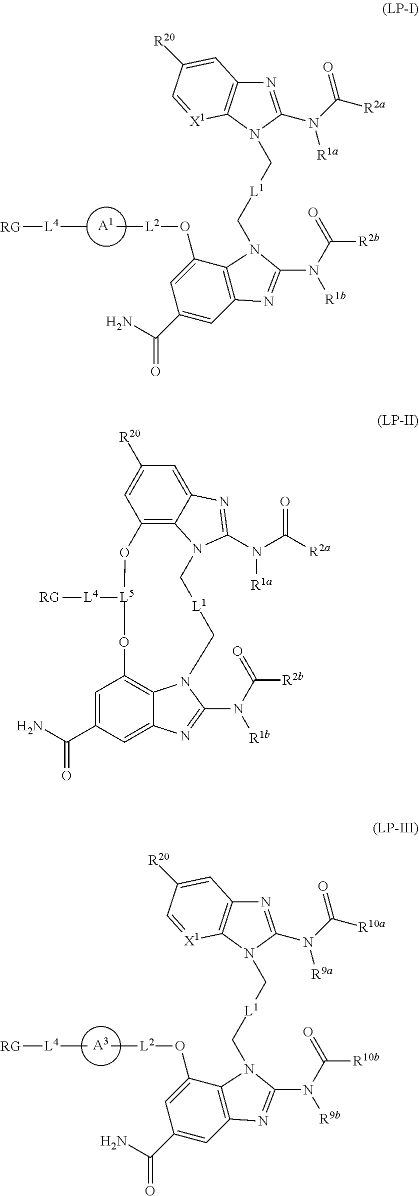

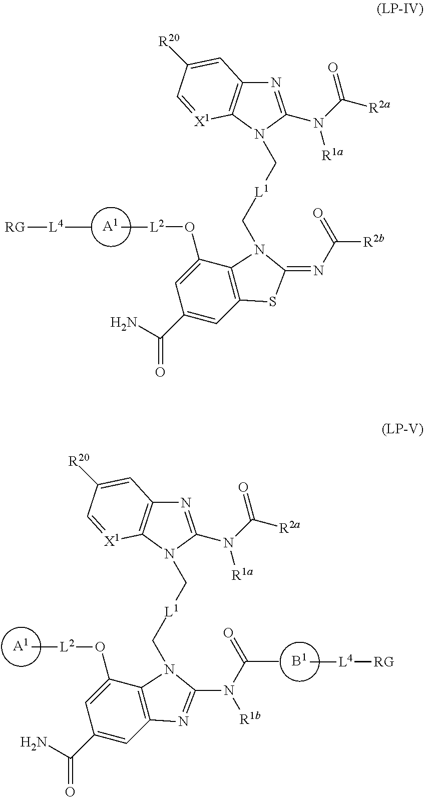

or a pharmaceutically acceptable salt thereof or tautomer thereof,

Ring A¹, Ring A³, Ring B¹, L¹, L², L⁴, L⁵, X¹, R^{1a}, R^{2a}, R^{1b}, R^{2b}, R²⁰, and RG are as defined herein.

Also provided herein are compounds of Formula (CONJ-I), (CONJ-II), (CONJ-III), (CONJ-IV), and (CONJ-V):

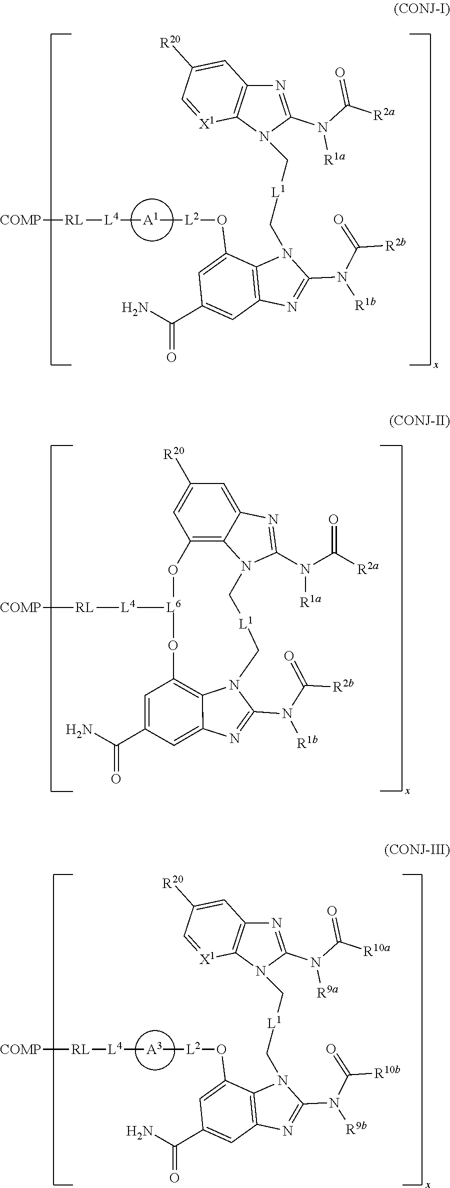

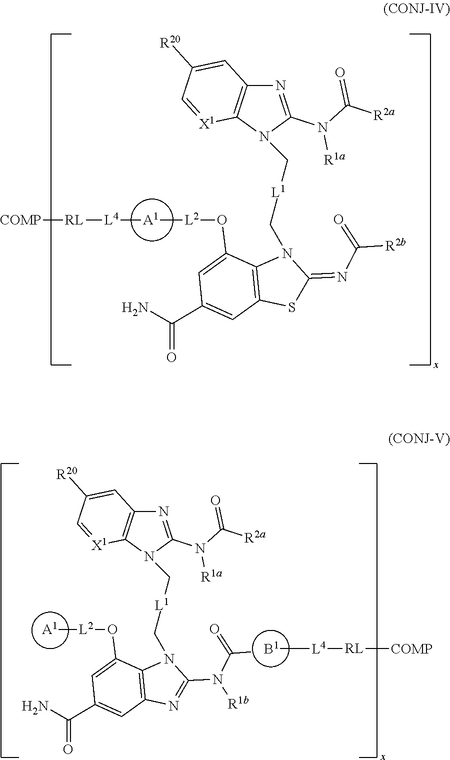

or a pharmaceutically acceptable salt thereof or tautomer thereof,

Ring A¹, Ring B¹, L¹, L², L⁴, L⁶, X¹, R^{1a}, R^{2a}, R^{1b}, R^{2b}, R²⁰, RL, COMP, and x are as defined herein.

In one embodiment of Formula (I), (II), (IV), (LP-I), (LP-II), (LP-IV), (LP-V), (LP-V), (CONJ-I), (CONJ-II), (CONJ-IV), or (CONJ-V), R^{1a} is hydrogen. In one embodiment of Formula (I), (II), (LP-I), (LP-II), (LP-V), (CONJ-I), (CONJ-II), or (CONJ-V), R^{1b} is hydrogen. In one embodiment of Formula (I), (II), (IV), (LP-I), (LP-II), (LP-IV), (LP-V), (LP-V), (CONJ-I), (CONJ-II), (CONJ-IV), or (CONJ-V), R^{1a} is C₁₋₆ alkyl, for example methyl. In one embodiment of Formula (I), (II), (LP-I), (LP-II), (LP-V), (CONJ-I), (CONJ-II), or (CONJ-V), R^{1b} is C₁₋₆ alkyl, for example methyl. In one embodiment of Formula (I), (II), (LP-I), (LP-II), (LP-V), (CONJ-I), (CONJ-II), or (CONJ-V), R^{1a} and R^{1b} are both hydrogen.

In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is a 3- to 12-membered heterocycle. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is a 5- to 6-membered heterocycle. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is a 3- to 12-membered heterocycle containing at least one N atom. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is a 5- to 6-membered heterocycle containing at least one N atom. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is a 3- to 12-membered heterocycle containing at least one N atom and the heterocycle is further substituted with at least one R⁵³. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is a 5- to 6-membered heterocycle containing at least one N atom and the heterocycle is further substituted with at least one R⁵³. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is a 3- to 12-membered heterocycle containing at least one N atom and the heterocycle is further substituted with at least one R⁵³ selected from C₁₋₆ alkyl, halogen, and haloC₁₋₆ alkyl. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is a 5- to 6-membered heterocycle containing at least one N atom and the heterocycle is further substituted with at least one R⁵³ selected from C₁₋₆ alkyl, halogen, and haloC₁₋₆ alkyl. In one embodiment of Formula (I), (II), (IV), (LP-I), (LP-II), (LP-IV), (CONJ-I), (CONJ-II), or (CONJ-IV), including any of the foregoing, R^{2b} is a 3- to 12-membered heterocycle containing at least one N atom. In one embodiment of Formula (I), (II), (IV), (LP-I), (LP-II), (LP-IV), (CONJ-I), (CONJ-II), or (CONJ-IV), including any of the foregoing, R^{2b} is a 5- to 6-membered heterocycle containing at least one N atom. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2b} is a 3- to 12-membered heterocycle containing at least one N atom and the heterocycle is further substituted with at least one R⁵³. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2b} is a 5- to 6-membered heterocycle containing at least one N atom and the heterocycle is further substituted with at least one R⁵³. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2b} is a 3- to 12-membered heterocycle containing at least one N atom and the heterocycle is further substituted with at least one R⁵³ selected from C₁₋₆ alkyl, halogen, and haloC₁₋₆ alkyl. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2b} is a 5- to 6-membered heterocycle containing at least one N atom and the heterocycle is further substituted with at least one R⁵³ selected from C₁₋₆ alkyl, halogen, and haloC₁₋₆ alkyl. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} and R^{2b}are both a 3- to 12-membered heterocycle containing at least one N atom. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} and R^{2b} are both a 5- to 6-membered heterocycle containing at least one N atom. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} and R^{2b} are both a 3- to 12-membered heterocycle containing at least one N atom and the heterocycle is further substituted with at least one R⁵³ selected from C₁₋₆ alkyl, halogen, and haloC₁₋₆ alkyl. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} and R^{2b} are both a 5- to 6-membered heterocycle containing at least one N atom and the heterocycle is further substituted with at least one R⁵³ selected from C₁₋₆ alkyl, halogen, and haloC₁₋₆ alkyl. In certain embodiments, R⁵³ is an amino acid residue.

In one embodiment of Formula (I), (II), (LP-I), (LP-II), (CONJ-I), or (CONJ-II), including any of the foregoing, R^{1a} is hydrogen, R^{1b} is hydrogen, and R^{2a} and R^{2b} are both a 3- to 12-membered heterocycle containing at least one N atom and the heterocycle is further substituted with at least one R⁵³ selected from C₁₋₆ alkyl, halogen, and haloC₁₋₆ alkyl. In certain embodiments, R⁵³ is an amino acid residue.

In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2a} and/or R^{2b} are independently selected from an optionally substituted pyrazole, an optionally substituted oxazole, an optionally substituted thiazole, an optionally substituted pyrrolidine, an optionally substituted phenyl, an optionally substituted pyridine, and an optionally substituted pyridazine. In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2a} and/or R^{2b} are independently selected from an optionally substituted pyrazole, an optionally substituted oxazole, an optionally substituted thiazole, an optionally substituted pyrrolidine, and an optionally substituted pyridazine. In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2a} and/or R^{2b} are independently selected from an optionally substituted pyrazole, an optionally substituted oxazole, and an optionally substituted thiazole.

In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2a} and/or R^{2b} are independently selected from

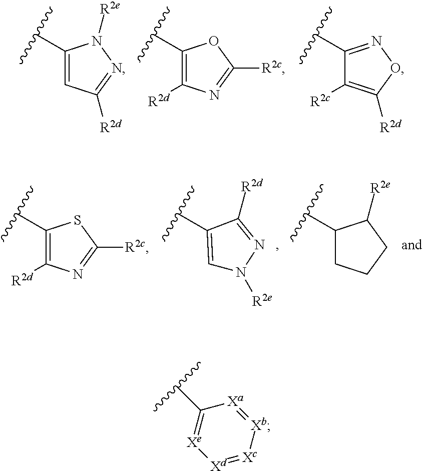

wherein R^{2c} and R^{2d} are independently hydrogen, halo, C₁₋₆ alkyl optionally substituted with one or more R⁵⁴, or 3- to 12-membered heterocycle;

R^{2e} is hydrogen or C₁₋₆ alkyl;

X^{a}, X^{b}, X^{c}, X^{d}, and X^{e} are independently selected from —N— and —CR^{2c}— wherein no more than two of X^{a}—X^{e} are N;

R⁵⁴ is independently selected from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), and —CN;

each R⁶¹ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle; and

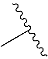

is the point of attachment to the rest of the compound.

In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2e} and R^{2d} are both C₁₋₆ alkyl. In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2e} is C₁₋₆ alkyl and R^{2d} is halo. In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2e} is C₁₋₆ alkyl and R^{2d} is C₁₋₆ alkyl optionally substituted with one or more R⁵⁴. In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2e} is C₁₋₆ alkyl and R^{2d} is haloC₁₋₆ alkyl. In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2e} is C₁₋₆ alkyl and R^{2d} is aminoC₁₋₆ alkyl. In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2e} is C₁₋₆ alkyl and R^{2d} is 3- to 12-membered heterocycle. In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2e} is C₁₋₆ alkyl and R^{2d} is hydrogen.

In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2c} and R^{2d} are both C₁₋₆ alkyl. In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2c} is hydrogen and R^{2d} is C₁₋₆ alkyl.

In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing,

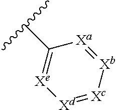

is selected from

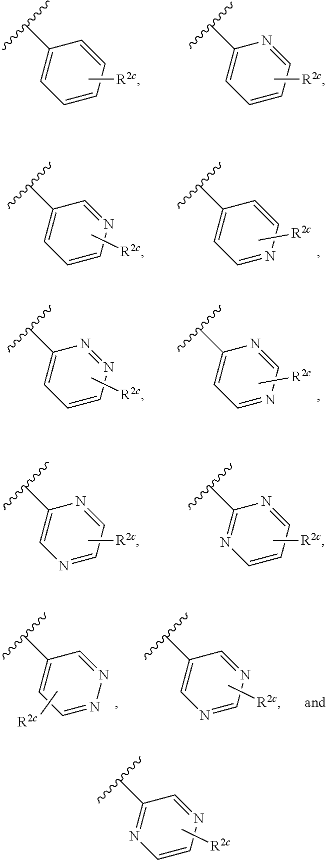

and R^{2c} is C₁₋₆ alkyl.

In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing,

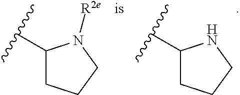

In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2a} and/or R^{2b} are independently selected from

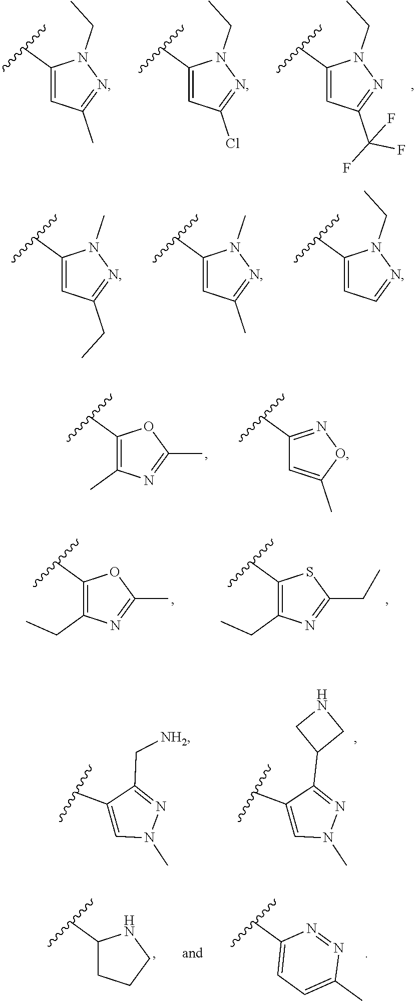

In one embodiment of Formula (I), (II), (IV), (LP-I), (LP-II), (LP-IV), (LP-V), (LP-V), (CONJ-I), (CONJ-II), (CONJ-IV), or (CONJ-V), including any of the foregoing, R^{2a} and/or R^{2b} are independently selected from

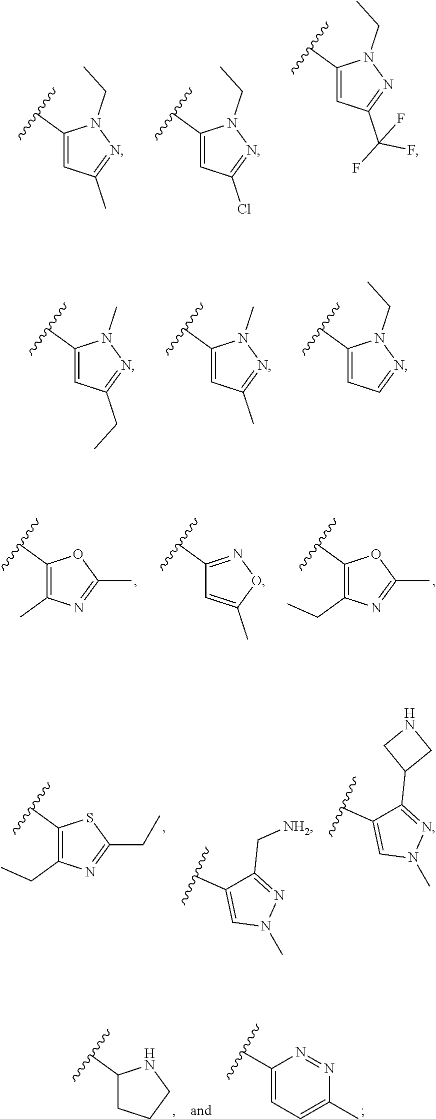

and R^{1a} and R^{1b} are both hydrogen.

In one embodiment of Formula (I), (II), (IV), (LP-I), (LP-II), (LP-IV), (CONJ-I), (CONJ-II), or (CONJ-IV), including any of the foregoing, R^{2a} and R^{2b} are both

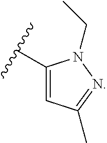 [SMILES: *c1cc(C)nn1CC]

In one embodiment of Formula (I), (II), (IV), (LP-I), (LP-II), (LP-IV), (CONJ-I), (CONJ-II), or (CONJ-IV), including any of the foregoing, R^{2a} and R^{2b} are both

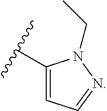 [SMILES: *c1ccnn1CC]

In one embodiment of Formula (I), (II), (IV), (LP-I), (LP-II), (LP-IV), (CONJ-I), (CONJ-II), or (CONJ-IV), including any of the foregoing, R^{2a} and R^{2b} are both

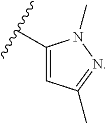 [SMILES: *c1cc(C)nn1C]

In one embodiment of Formula (I), (II), (IV), (LP-I), (LP-II), (LP-IV), (CONJ-I), (CONJ-II), or (CONJ-IV), including any of the foregoing, R^{2a} and R^{2b} are both

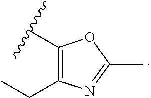 [SMILES: *c1oc(C)nc1CC ; "."]

In one embodiment of Formula (I), (II), (IV), (LP-I), (LP-II), (LP-IV), (CONJ-I), (CONJ-II), or (CONJ-IV), including any of the foregoing, R^{2a} and R^{2b} are both

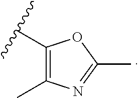 [SMILES: *c1oc(C)nc1C ; "."]

In one embodiment of Formula (I), (II), (IV), (LP-I), (LP-II), (LP-IV), (CONJ-I), (CONJ-II), or (CONJ-IV), including any of the foregoing, R^{2a} and R^{2b} are both

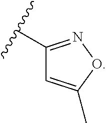 [SMILES: *c1cc(C)on1]

In one embodiment of Formula (I), (II), (IV), (LP-I), (LP-II), (LP-IV), (CONJ-I), (CONJ-II), or (CONJ-IV), including any of the foregoing, R^{2a} is

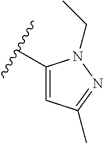 [SMILES: *c1cc(C)nn1CC]

### and R^{2b} is

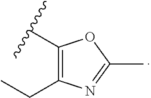 [SMILES: *c1oc(C)nc1CC ; "."]

In one embodiment of Formula (I), (II), (IV), (LP-I), (LP-II), (LP-IV), (CONJ-I), (CONJ-II), or (CONJ-IV), including any of the foregoing, R^{2a} is

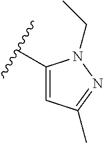 [SMILES: *c1cc(C)nn1CC]

### and R^{2b} is

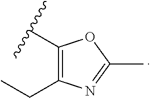 [SMILES: *c1oc(C)nc1CC ; "."]

In one embodiment of Formula (I), (II), (IV), (LP-I), (LP-II), (LP-IV), (CONJ-I), (CONJ-II), or (CONJ-IV), including any of the foregoing, R^{2b} is

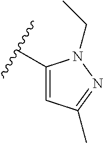 [SMILES: *c1cc(C)nn1CC]

### and R^{2a} is

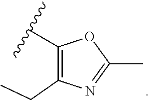 [SMILES: *c1oc(C)nc1CC]

In one embodiment of Formula (I), (II), (IV), (LP-I), (LP-II), (LP-IV), (CONJ-I), (CONJ-II), or (CONJ-IV), including any of the foregoing, R^{2a} is

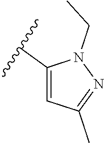 [SMILES: *c1cc(C)nn1CC]

### and R^{2b} is

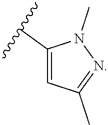 [SMILES: *c1cc(C)nn1C]

In one embodiment of Formula (I), (II), (IV), (LP-I), (LP-II), (LP-IV), (CONJ-I), (CONJ-II), or (CONJ-IV), including any of the foregoing, R^{2a} is

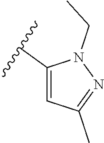 [SMILES: *c1cc(C)nn1CC]

### and R^{2b} is

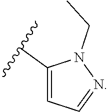 [SMILES: *c1ccnn1CC]

In one embodiment of Formula (I), (II), (IV), (LP-I), (LP-II), (LP-IV), (CONJ-I), (CONJ-II), or (CONJ-IV), including any of the foregoing, R^{2a} is

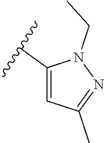 [SMILES: *c1cc(C)nn1CC]

### and R^{2b} is

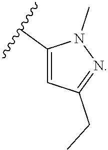 [SMILES: *c1cc(CC)nn1C]

In one embodiment of Formula (I), (II), (IV), (LP-I), (LP-II), (LP-IV), (CONJ-I), (CONJ-II), or (CONJ-IV), including any of the foregoing, R^{2a} is

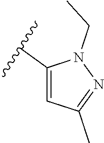 [SMILES: *c1cc(C)nn1CC]

### and R^{2b} is

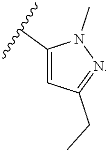 [SMILES: *c1cc(CC)nn1C]

In one embodiment of Formula (I), (II), (IV), (LP-I), (LP-II), (LP-IV), (CONJ-I), (CONJ-II), or (CONJ-IV), including any of the foregoing, R^{2a} is

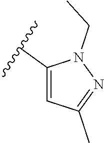 [SMILES: *c1cc(C)nn1CC]

### and R^{2b} is

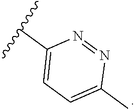 [SMILES: *c1ccc(C)nn1]

In one embodiment of Formula (I), (II), (IV), (LP-I), (LP-II), (LP-IV), (CONJ-I), (CONJ-II), or (CONJ-IV), including any of the foregoing, R^{2a} is

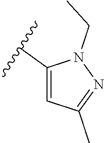 [SMILES: *c1cc(C)nn1CC]

### and R^{2b} is

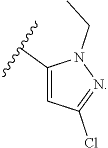 [SMILES: *c1cc(Cl)nn1CC]

In one embodiment of Formula (I), (II), (IV), (LP-I), (LP-II), (LP-IV), (CONJ-I), (CONJ-II), or (CONJ-IV), including any of the foregoing, R^{2a} is

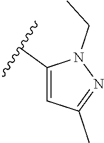 [SMILES: *c1cc(C)nn1CC]

### and R^{2b} is

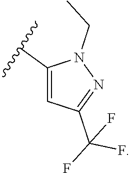 [SMILES: *c1cc(C(F)(F)F)nn1CC]

In one embodiment of Formula (I), (II), (IV), (LP-I), (LP-II), (LP-IV), (CONJ-I), (CONJ-II), or (CONJ-IV), including any of the foregoing, R^{2a} is

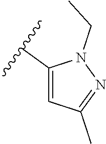 [SMILES: *c1cc(C)nn1CC]

### and R^{2b} is

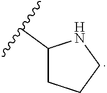 [SMILES: *C1CCCN1]

In one embodiment of Formula (I), (II), (IV), (LP-I), (LP-II), (LP-IV), (CONJ-I), (CONJ-II), or (CONJ-IV), including any of the foregoing, R^{2a} is

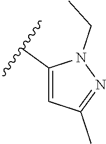 [SMILES: *c1cc(C)nn1CC]

### and R^{2b} is

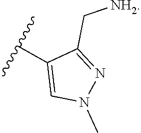 [SMILES: *c1cn(C)nc1CN]

In one embodiment of Formula (I), (II), (IV), (LP-I), (LP-II), (LP-IV), (CONJ-I), (CONJ-II), or (CONJ-IV), including any of the foregoing, R^{2a} is

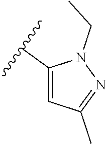 [SMILES: *c1cc(C)nn1CC]

### and R^{2b} is

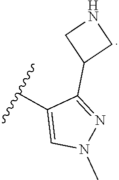 [SMILES: *c1cn(C)nc1C1CNC1]

In one embodiment of Formula (I), (II), (LP-I), (LP-II), (CONJ-I), or (CONJ-II), including any of the foregoing, R^{2a} and R^{2b} are both

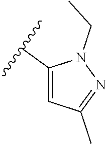 [SMILES: *c1cc(C)nn1CC]

and R^{1a} and R^{1b} are both hydrogen. In one embodiment of Formula (I), (II), (LP-I), (LP-II), (CONJ-I), or (CONJ-II), including any of the foregoing, R^{2a} and R^{2b} are both

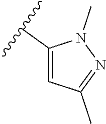 [SMILES: *c1cc(C)nn1C]

and R^{1a} and R^{1b} are both hydrogen. In one embodiment of Formula (I), (II), (LP-I), (LP-II), (CONJ-I), or (CONJ-II), including any of the foregoing, R^{2a} and R^{2b} are both

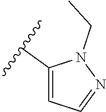 [SMILES: *c1ccnn1CC]

and R^{1a} and R^{1b} are both hydrogen. In one embodiment of Formula (I), (II), (LP-I), (LP-II), (CONJ-I), or (CONJ-II), including any of the foregoing, R^{2a} and R^{2b} are both

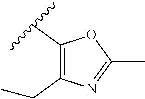 [SMILES: *c1oc(C)nc1CC]

and R^{1a} and R^{1b} are both hydrogen. In one embodiment of Formula (I), (II), (LP-I), (LP-II), (CONJ-I), or (CONJ-II), including any of the foregoing, R^{2a} and R^{2b} are both

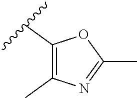 [SMILES: *c1oc(C)nc1C]

and R^{1a} and R^{1b} are both hydrogen. In one embodiment of Formula (I), (II), (LP-I), (LP-II), (CONJ-I), or (CONJ-II), including any of the foregoing, R^{2a} and R^{2b} are both

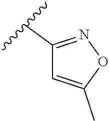 [SMILES: *c1cc(C)on1]

and R^{1a} and R^{1b} are both hydrogen. In one embodiment of Formula (I), (II), (LP-I), (LP-II), (CONJ-I), or (CONJ-II), including any of the foregoing, R^{2a} is

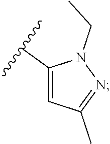 [SMILES: *c1cc(C)nn1CC]

### R^{2b} is

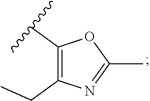 [SMILES: *c1oc(C)nc1CC]

and R^{1a} and R^{1b} are both hydrogen. In one embodiment of Formula (I), (II), (LP-I), (LP-II), (CONJ-I), or (CONJ-II), including any of the foregoing, R^{2a} is

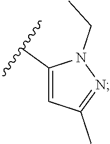 [SMILES: *c1cc(C)nn1CC]

### R^{2b} is

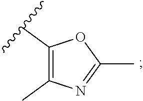 [SMILES: *c1oc(C)nc1C]

and R^{1a} and R^{1b} are both hydrogen. In one embodiment of Formula (I), (II), (LP-I), (LP-II), (CONJ-I), or (CONJ-II), including any of the foregoing, R^{2b} is

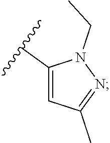 [SMILES: *c1cc(C)nn1CC]

### R^{2a} is

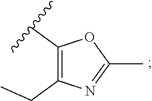 [SMILES: *c1oc(C)nc1CC]

and R^{1a} and R^{1b} are both hydrogen. In one embodiment of Formula (I), (II), (LP-I), (LP-II), (CONJ-I), or (CONJ-II), including any of the foregoing, R^{2a} is

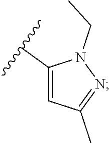 [SMILES: *c1cc(C)nn1CC]

### R^{2b} is

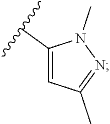 [SMILES: *c1cc(C)nn1C]

and R^{1a} and R^{1b} are both hydrogen. In one embodiment of Formula (I), (II), (LP-I), (LP-II), (CONJ-I), or (CONJ-II), including any of the foregoing, R^{2a} is

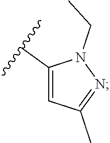 [SMILES: *c1cc(C)nn1CC]

### R^{2b} is

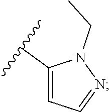 [SMILES: *c1ccnn1CC]

and R^{1a} and R^{1b} are both hydrogen. In one embodiment of Formula (I), (II), (LP-I), (LP-II), (CONJ-I), or (CONJ-II), including any of the foregoing, R^{2a} is

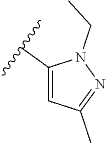 [SMILES: *c1cc(C)nn1CC]

### and R^{2b} is

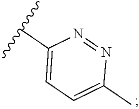 [SMILES: *c1ccc(C)nn1]

and R^{1a} and R^{1b} are both hydrogen. In one embodiment of Formula (I), (II), (LP-I), (LP-II), (CONJ-I), or (CONJ-II), including any of the foregoing, R^{2a} is

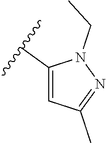 [SMILES: *c1cc(C)nn1CC]

### and R^{2b} is

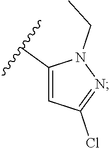 [SMILES: *c1cc(Cl)nn1CC]

and R^{1a} and R^{1b} are both hydrogen. In one embodiment of Formula (I), (II), (LP-I), (LP-II), (CONJ-I), or (CONJ-II), including any of the foregoing, R^{2a} is

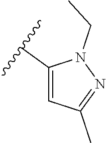 [SMILES: *c1cc(C)nn1CC]

### and R^{2b} is

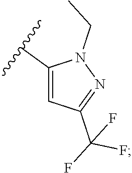 [SMILES: *c1cc(C(F)(F)F)nn1CC]

and R^{1a} and R^{1b}are both hydrogen. In one embodiment of Formula (I), (II), (LP-I), (LP-II), (CONJ-I), or (CONJ-II), including any of the foregoing, R^{2a} is

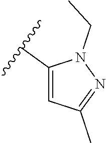 [SMILES: *c1cc(C)nn1CC]

### and R^{2b} is

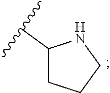 [SMILES: *C1CCCN1]

and R^{1a} and R^{1b} are both hydrogen. In one embodiment of Formula (I), (II), (LP-I), (LP-II), (CONJ-I), or (CONJ-II), including any of the foregoing, R^{2a} is

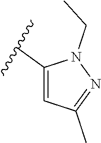 [SMILES: *c1cc(C)nn1CC]

### and R^{2b} is

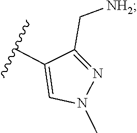 [SMILES: *c1cn(C)nc1CN]

and R^{1a} and R^{1b} are both hydrogen. In one embodiment of Formula (I), (II), (LP-I), (LP-II), (CONJ-I), or (CONJ-II), including any of the foregoing, R^{2a} is

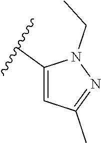 [SMILES: *c1cc(C)nn1CC]

### and R^{2b} is

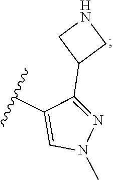 [SMILES: *c1cn(C)nc1C1CNC1]

and R^{1a} and R^{1b} are both hydrogen.

In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is optionally substituted C₁₋₆ alkyl, wherein the C₁₋₆ alkyl is optionally substituted with one or more R⁵¹. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is optionally substituted C₁₋₆ alkyl, wherein the C₁₋₆ alkyl is optionally substituted with one R⁵¹ and R⁵¹ is 3- to 12-membered heterocycle optionally substituted with one or more R⁵². In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is optionally substituted C₁₋₆ alkyl, wherein the C₁₋₆ alkyl is optionally substituted with one R⁵¹ and R⁵¹ is 5- to 6-membered heterocycle optionally substituted with one or more R⁵². In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is —CH₂—R⁵¹ wherein R⁵¹ is 3- to 12-membered heterocycle optionally substituted with one or more R⁵² and R⁵² is C₁₋₆ alkyl. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is —CH₂—R⁵¹ wherein R⁵¹ is 5- to 6-membered heterocycle optionally substituted with one or more R⁵² and R⁵² is C₁₋₆ alkyl. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2a} is —CH₂—R⁵¹ wherein R⁵¹ is an optionally substituted pyrazole, an optionally substituted oxazole, or an optionally substituted thiazole.

In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2b} is optionally substituted C₁₋₆ alkyl, wherein the C₁₋₆ alkyl is optionally substituted with one or more R⁵¹. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2b} is optionally substituted C₁₋₆ alkyl, wherein the C₁₋₆ alkyl is optionally substituted with one R⁵ and R⁵¹ is 3- to 12-membered heterocycle optionally substituted with one or more R⁵². In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2b} is optionally substituted C₁₋₆ alkyl, wherein the C₁₋₆ alkyl is optionally substituted with one R⁵¹ and R⁵¹ is 5- to 6-membered heterocycle optionally substituted with one or more R⁵². In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2b} is —CH₂—R⁵¹ wherein R⁵¹ is 3- to 12-membered heterocycle optionally substituted with one or more R⁵² and R⁵² is C₁₋₆ alkyl. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2b} is —CH₂—R⁵¹ wherein R⁵¹ is 5- to 6-membered heterocycle optionally substituted with one or more R⁵² and R⁵² is C₁₋₆ alkyl. In one embodiment of Formula (I), (II), (IV), (LP-I), (LP-II), (LP-IV), (LP-V), (CONJ-I), (CONJ-II), (CONJ-IV), or (CONJ-V), including any of the foregoing, R^{2b} is —CH₂—R⁵¹ wherein R⁵¹ is an optionally substituted pyrazole, an optionally substituted oxazole, or an optionally substituted thiazole.

In one embodiment of Formula (I), (II), (LP-I), (LP-II), (LP-IV), (CONJ-I), (CONJ-II), (CONJ-IV), including any of the foregoing, R^{2a} is optionally substituted C₁₋₆ alkyl, wherein the C₁₋₆ alkyl is optionally substituted with one R⁵¹ and R⁵¹ is 3- to 12-membered heterocycle optionally substituted with one or more R⁵² and R^{2b} is a 3- to 12-membered heterocycle containing at least one N atom and the heterocycle is further substituted with at least one R⁵³. In one embodiment of Formula (I), (II), (LP-I), (LP-II), (LP-IV), (CONJ-I), (CONJ-II), (CONJ-IV), including any of the foregoing, R^{2a} is optionally substituted C₁. 6 alkyl, wherein the C₁₋₆ alkyl is optionally substituted with one R⁵¹ and R⁵¹ is 5- to 6-membered heterocycle optionally substituted with one or more R⁵² and R^{2b} is a 5- to 6-membered heterocycle containing at least one N atom and the heterocycle is further substituted with at least one R⁵³. In one embodiment of Formula (I), (II), (LP-I), (LP-II), (LP-IV), (CONJ-I), (CONJ-II), (CONJ-IV), including any of the foregoing, R^{2a} is optionally substituted C₁₋₆ alkyl, wherein the C₁₋₆ alkyl is optionally substituted with one R⁵¹ and R⁵¹ is an optionally substituted pyrazole, an optionally substituted oxazole, or an optionally substituted thiazole and R^{2b} is an optionally substituted pyrazole, an optionally substituted oxazole, or an optionally substituted thiazole. In certain embodiments, R⁵³ is an amino acid residue.

In one embodiment of Formula (I), (II), (LP-I), (LP-II), (LP-IV), (CONJ-I), (CONJ-II), (CONJ-IV), including any of the foregoing, R^{2b} is optionally substituted C₁₋₆ alkyl, wherein the C₁₋₆ alkyl is optionally substituted with one R⁵¹ and R⁵¹ is 3- to 12-membered heterocycle optionally substituted with one or more R⁵² and R^{2a} is a 3- to 12-membered heterocycle containing at least one N atom and the heterocycle is further substituted with at least one R⁵³. In one embodiment of Formula (I), (II), (LP-I), (LP-II), (LP-IV), (CONJ-I), (CONJ-II), (CONJ-IV), including any of the foregoing, R^{2b} is optionally substituted C₁. 6 alkyl, wherein the C₁₋₆ alkyl is optionally substituted with one R⁵¹ and R⁵¹ is 5- to 6-membered heterocycle optionally substituted with one or more R⁵² and R^{2a} is a 5- to 6-membered heterocycle containing at least one N atom and the heterocycle is further substituted with at least one R⁵³. In one embodiment of Formula (I), (II), (LP-I), (LP-II), (LP-IV), (CONJ-I), (CONJ-II), (CONJ-IV), including any of the foregoing, R^{2b} is optionally substituted C₁₋₆ alkyl, wherein the C₁₋₆ alkyl is optionally substituted with one R⁵¹ and an optionally substituted pyrazole, an optionally substituted oxazole, or an optionally substituted thiazole and R^{2a} is an optionally substituted pyrazole, an optionally substituted oxazole, or an optionally substituted thiazole. In certain embodiments, R⁵³ is an amino acid residue.

In one embodiment of Formula (I), (II), (LP-I), (LP-II), (LP-IV), (CONJ-I), (CONJ-II), (CONJ-IV), including any of the foregoing, R^{2a} and/or R^{2b} are independently selected from

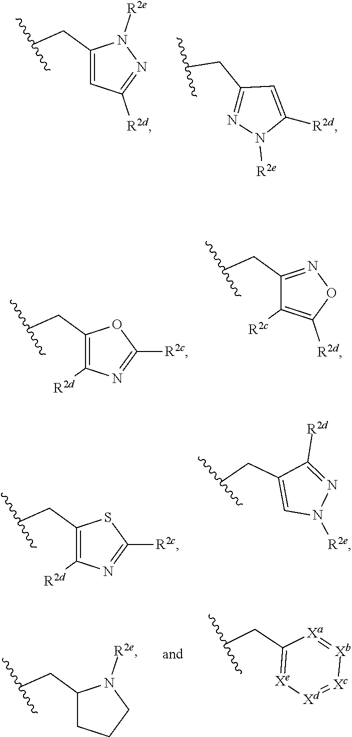

wherein R^{2c}, R^{2d}, R^{2e} and X^{a}—X^{e} are as defined herein.

In one embodiment of Formula (I), (II), (LP-I), (LP-II), (LP-IV), (CONJ-I), (CONJ-II), (CONJ-IV), including any of the foregoing, R^{2b} is selected from

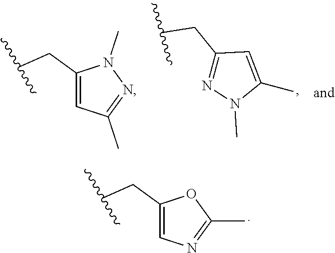

In one embodiment of Formula (I), (II), (LP-I), (LP-II), (CONJ-I), or (CONJ-II), including any of the foregoing, R^{2b} is selected from

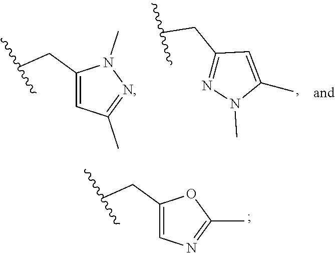

and R^{1b} is hydrogen.

In one embodiment of Formula (I), (II), (LP-I), (LP-II), (LP-IV), (CONJ-I), (CONJ-II), (CONJ-IV), including any of the foregoing, R^{2a} is selected from

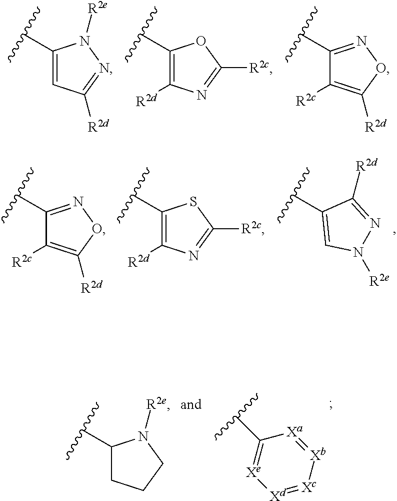

and R^{2b} is selected from

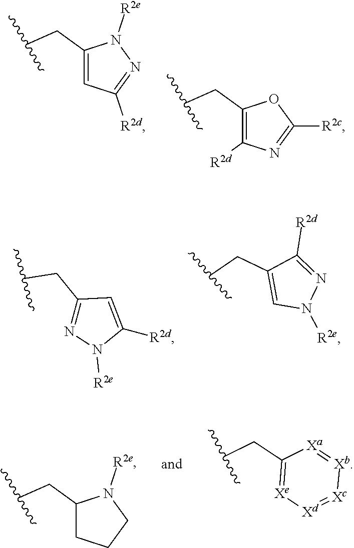

In one embodiment of Formula (I), (II), (LP-I), (LP-II), (LP-IV), (CONJ-I), (CONJ-II), (CONJ-IV), including any of the foregoing, R^{2a} is selected from

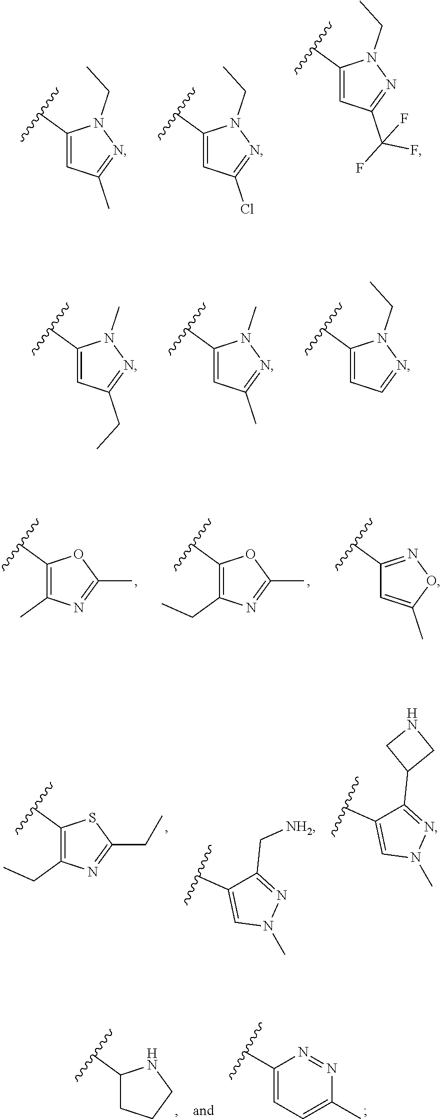

and R^{2b} is selected from

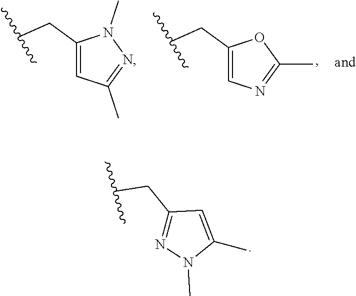

In one embodiment of Formula (I), (II), (LP-I), (LP-II), (LP-IV), (CONJ-I), (CONJ-II), (CONJ-IV), including any of the foregoing, R^{2a} is

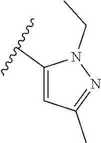 [SMILES: *c1cc(C)nn1CC]

and R^{2b} is selected from

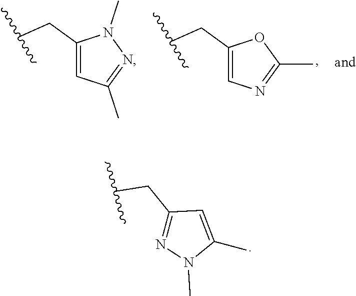

In one embodiment of Formula (I), (II), (LP-I), (LP-II), (LP-IV), (CONJ-I), (CONJ-II), (CONJ-IV), including any of the foregoing, R^{2a} is

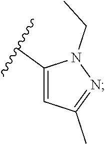 [SMILES: *c1cc(C)nn1CC]

R^{2b} is selected from

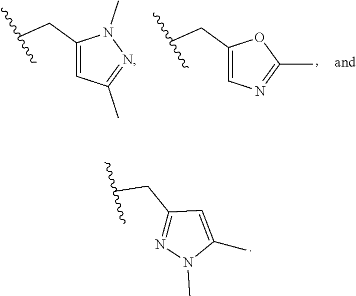

and R^{1a} and R^{1b} are both hydrogen.

In one embodiment of Formula (I), (II), (LP-I), (LP-II), (LP-IV), (CONJ-I), (CONJ-II), (CONJ-IV), including any of the foregoing, R^{2a} is

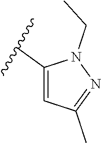 [SMILES: *c1cc(C)nn1CC]

### and R^{2b} is

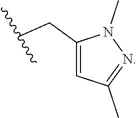 [SMILES: *Cc1cc(C)nn1C]

In one embodiment of Formula (I), (II), (LP-I), (LP-II), (LP-IV), (CONJ-I), (CONJ-II), (CONJ-IV), including any of the foregoing, R^{2a} is

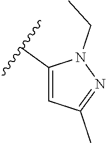 [SMILES: *c1cc(C)nn1CC]

### and R^{2b} is

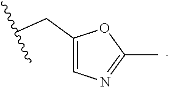 [SMILES: *Cc1cnc(C)o1]

In one embodiment of Formula (I), (II), (LP-I), (LP-II), (LP-IV), (CONJ-I), (CONJ-II), (CONJ-IV), including any of the foregoing, R^{2a} is

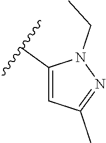 [SMILES: *c1cc(C)nn1CC]

### and R^{2b} is

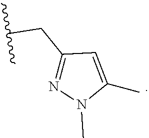 [SMILES: *Cc1cc(C)n(C)n1]

In one embodiment of Formula (I), (II), (LP-I), (LP-II), (CONJ-I), or (CONJ-II), including any of the foregoing, R^{2a} is

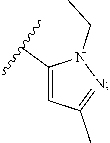 [SMILES: *c1cc(C)nn1CC]

### R^{2b} is

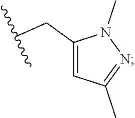 [SMILES: *Cc1cc(C)nn1C]

and R^{1a} and R^{1b} are both hydrogen. In one embodiment of Formula (I), (II), (LP-I), (LP-II), (CONJ-I), or (CONJ-II), including any of the foregoing, R^{2a} is

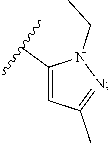 [SMILES: *c1cc(C)nn1CC]

### R^{2b} is

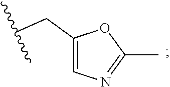 [SMILES: *Cc1cnc(C)o1]

and R^{1a} and R^{1b} are both hydrogen. In one embodiment of Formula (I), (II), (LP-I), (LP-II), (CONJ-I), or (CONJ-II), including any of the foregoing, R^{2a} is

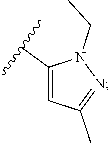 [SMILES: *c1cc(C)nn1CC]

### R^{2b} is

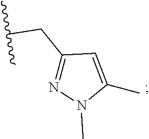 [SMILES: *Cc1cc(C)n(C)n1]

and R^{1a} and R^{1b} are both hydrogen.

In one embodiment of Formula (I), (II), (LP-I), (LP-II), (CONJ-I), or (CONJ-II), including any of the foregoing, R^{1b} and R^{2b} are joined together with the atoms to which they are attached to form an optionally substituted 3- to 12-membered heterocycle wherein the heterocycle is optionally substituted with one or more R⁵³. In one embodiment of Formula (I), (II), (LP-I), (LP-II), (CONJ-I), or (CONJ-II), including any of the foregoing, R^{1b} and R^{2b} are joined together with the atoms to which they are attached to form an optionally substituted 8-to 12-membered heterocycle wherein the heterocycle is optionally substituted with one or more R⁵³. In one embodiment of Formula (I), (II), (LP-I), (LP-II), (CONJ-I), or (CONJ-II), including any of the foregoing, R^{1b} and R^{2b} are joined together with the atoms to which they are attached to form an optionally substituted N—C(O)-linked 3- to 12-membered fused heterocycle. In one embodiment of Formula (I), (II), (LP-I), (LP-II), (CONJ-I), or (CONJ-II), including any of the foregoing, R^{1b} and R^{2b} are joined together with the atoms to which they are attached to form an optionally substituted N—C(O)-linked 8- to 12-membered fused heterocycle.

In one embodiment of Formula (I), (II), (LP-I), (LP-II), (CONJ-I), or (CONJ-II), including any of the foregoing, R^{1a} and R^{2a} are joined together with the atoms to which they are attached to form an optionally substituted 3- to 12-membered heterocycle wherein the heterocycle is optionally substituted with one or more R⁵³. In one embodiment of Formula (I), (II), (LP-I), (LP-II), (CONJ-I), or (CONJ-II), including any of the foregoing, R^{1a} and R^{2a} are joined together with the atoms to which they are attached to form an optionally substituted 8-to 12-membered heterocycle wherein the heterocycle is optionally substituted with one or more R⁵³. In one embodiment of Formula (I), (II), (LP-I), (LP-II), (CONJ-I), or (CONJ-II), including any of the foregoing, R^{1a} and R^{2a} are joined together with the atoms to which they are attached to form an optionally substituted N—C(O)-linked 3- to 12-membered fused heterocycle. In one embodiment of Formula (I), (II), (LP-I), (LP-II), (CONJ-I), or (CONJ-II), including any of the foregoing, R^{1a} and R^{2a} are joined together with the atoms to which they are attached to form an optionally substituted N—C(O)-linked 8- to 12-membered fused heterocycle.

In one embodiment of Formula (I), (II), (LP-I), (LP-II), (CONJ-I), or (CONJ-II), including any of the foregoing, R^{1b} and R^{2b} are joined together with the atoms to which they are attached to form an optionally substituted 3- to 12-membered heterocycle wherein the heterocycle is optionally substituted with one or more R⁵³, R^{1a} is hydrogen or C₁₋₆ alkyl, and R^{2a} is a 3- to 12-membered heterocycle, which is optionally substituted with one or more R⁵³. In one embodiment of Formula (I), (II), (LP-I), (LP-II), (CONJ-I), or (CONJ-II), including any of the foregoing, R^{1a} and R^{2a} are joined together with the atoms to which they are attached to form an optionally substituted 3- to 12-membered heterocycle wherein the heterocycle is optionally substituted with one or more R⁵³, R^{1b} is hydrogen or C₁₋₆ alkyl, and R^{2b} is a 3- to 12-membered heterocycle, which is optionally substituted with one or more R⁵³. In one embodiment of Formula (I), (II), (LP-I), (LP-II), (CONJ-I), or (CONJ-II), including any of the foregoing, R^{1b} and R^{2b} are joined together with the atoms to which they are attached to form an optionally substituted 3- to 12-membered heterocycle wherein the heterocycle is optionally substituted with one or more R⁵³ and R^{1a} and R^{2a} are joined together with the atoms to which they are attached to form an optionally substituted 3- to 12-membered heterocycle wherein the heterocycle is optionally substituted with one or more R⁵³.

In one embodiment of Formula (I), (II), (LP-I), (LP-II), (CONJ-I), or (CONJ-II), including any of the foregoing, the optionally substituted 3- to 12-membered heterocycle is an optionally substituted N—C(O)-linked 3- to 12-membered fused heterocycle. In one embodiment of Formula (I), (II), (LP-I), (LP-II), (CONJ-I), or (CONJ-II), including any of the foregoing, the optionally substituted 3- to 12-membered heterocycle is an optionally substituted N—C(O)-linked 8- to 12-membered fused heterocycle.

In one embodiment of Formula (III), (LP-III), or (CONJ-III), including any of the foregoing, R^{9a} and R^{10a} are joined together with the atoms to which they are attached to form an optionally substituted 3- to 12-membered heterocycle wherein the heterocycle is optionally substituted with one or more R⁵³, R^{9b} is hydrogen or C₁₋₆ alkyl, and R^{10b} is a 3- to 12-membered heterocycle, which is optionally substituted with one or more R⁵³. In one embodiment of Formula (III), (LP-III), or (CONJ-III), including any of the foregoing, R^{9b} and R^{10b} are joined together with the atoms to which they are attached to form an optionally substituted 3- to 12-membered heterocycle wherein the heterocycle is optionally substituted with one or more R⁵³, R^{9a} is hydrogen or C₁₋₆ alkyl, and R^{10a} is a 3- to 12-membered heterocycle, which is optionally substituted with one or more R⁵³. In one embodiment of Formula (III), (LP-III), or (CONJ-III), including any of the foregoing, R^{9a} and R^{10a} are joined together with the atoms to which they are attached to form an optionally substituted 3- to 12-membered heterocycle wherein the heterocycle is optionally substituted with one or more R⁵³ and R^{9b} and R^{10b} are joined together with the atoms to which they are attached to form an optionally substituted 3- to 12-membered heterocycle wherein the heterocycle is optionally substituted with one or more R⁵³.

In one embodiment of Formula (III), (LP-III), or (CONJ-III), including any of the foregoing, the optionally substituted 3- to 12-membered heterocycle is an optionally substituted N—C(O)-linked 3- to 12-membered fused heterocycle.

In one embodiment of Formula (I)—(IV), (LP-I), (LP-V), or (CONJ-I)—(CONJ-V), including any of the foregoing, the optionally substituted N—C(O)-linked 3- to 12-membered fused heterocycle is an optionally substituted 5-5 fused ring system, an optionally substituted 5-6 fused ring system, an optionally substituted 6-6 fused ring system, or an optionally substituted 5-7 fused ring system.

In one embodiment of Formula (I)—(IV), (LP-I), (LP-V), or (CONJ-I)—(CONJ-V), including any of the foregoing, the optionally substituted N—C(O)-linked 3- to 12-membered fused heterocycle is independently selected from the formula:

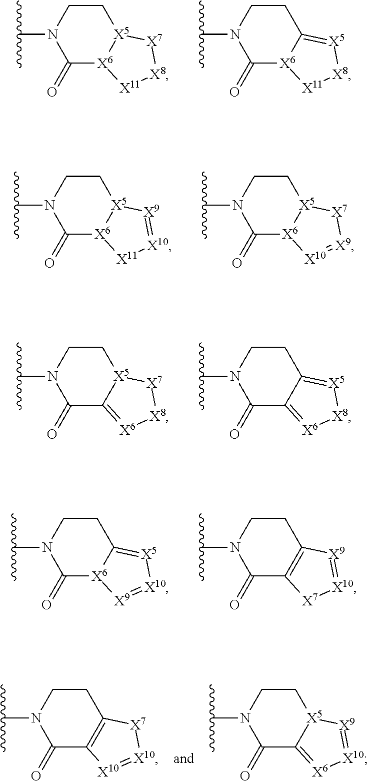

wherein X⁵, X⁶, X⁹, X¹⁰ are independently N or CR¹¹;

X⁷, X⁸, and X¹¹ are independently O, NH or CHR¹¹;

R¹¹ is hydrogen or C₁₋₆ alkyl; and

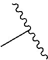

is the point of attachment to the rest of the compound;

wherein if four of any one of X⁵—X¹¹ are present, at least one is CR¹¹ or CHR¹¹.

In one embodiment of Formula (I)—(IV), (LP-I), (LP-V), or (CONJ-I)—(CONJ-V), including any of the foregoing, the optionally substituted N—C(O)-linked 3- to 12-membered fused heterocycle is independently selected from the formula:

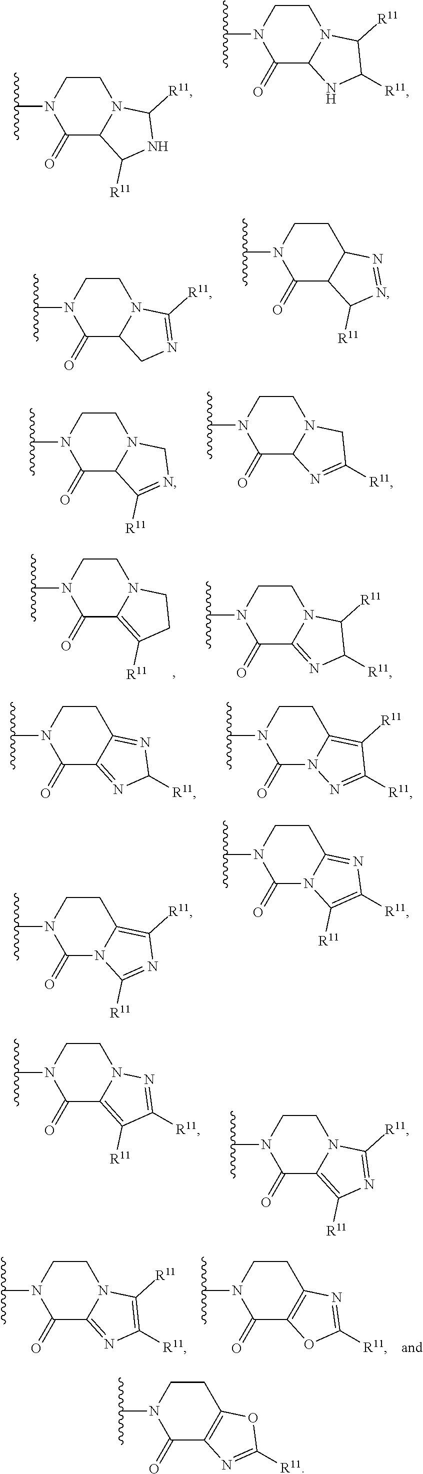

In one embodiment of Formula (I)—(IV), (LP-I), (LP-V), or (CONJ-I)—(CONJ-V), including any of the foregoing, the optionally substituted N—C(O)-linked 3- to 12-membered fused heterocycle is independently selected from the formula:

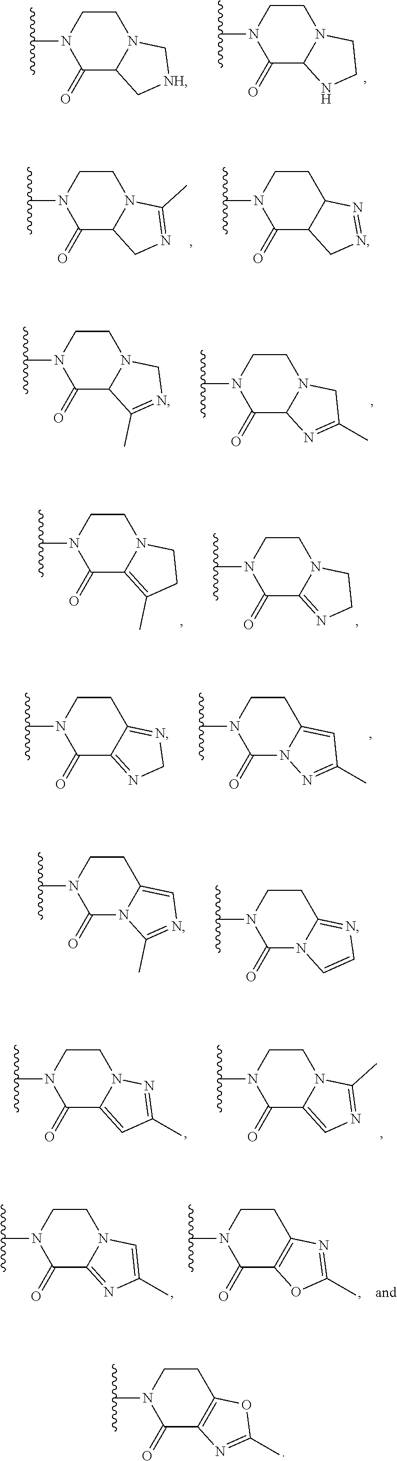

In one embodiment of Formula (I)—(IV), (LP-I), (LP-V), or (CONJ-I)—(CONJ-V), including any of the foregoing, the optionally substituted N—C(O)-linked 3- to 12-membered fused heterocycle is independently selected from the formula:

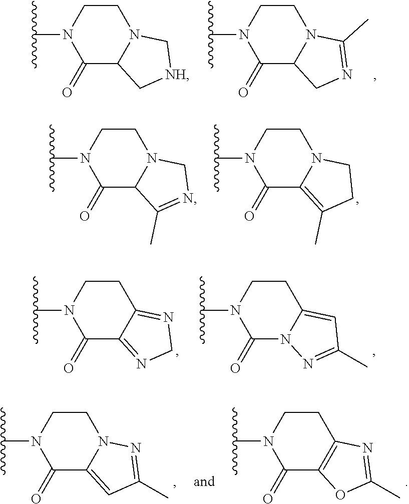

In one embodiment of Formula (I)—(IV), (LP-I), (LP-V), or (CONJ-I)—(CONJ-V), including any of the foregoing, the optionally substituted N—C(O)-linked 3- to 12-membered fused heterocycle is

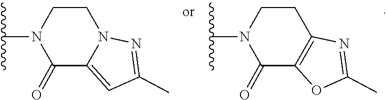 [SMILES: *N1CCn2nc(C)cc2C1=O ; *N1CCc2nc(C)oc2C1=O]

In one embodiment of Formula (I), (II), (LP-I), (LP-II), (CONJ-I), or (CONJ-II), including any of the foregoing, R^{1a} is hydrogen or C₁₋₆ alkyl; R^{2a} is a 3- to 12-membered heterocycle containing at least one N atom and the heterocycle is further substituted with at least one R⁵³; and R^{1b} and R^{2b} are joined together with the atoms to which they are attached to form an optionally substituted N—C(O)-linked 3- to 12-membered fused heterocycle selected from:

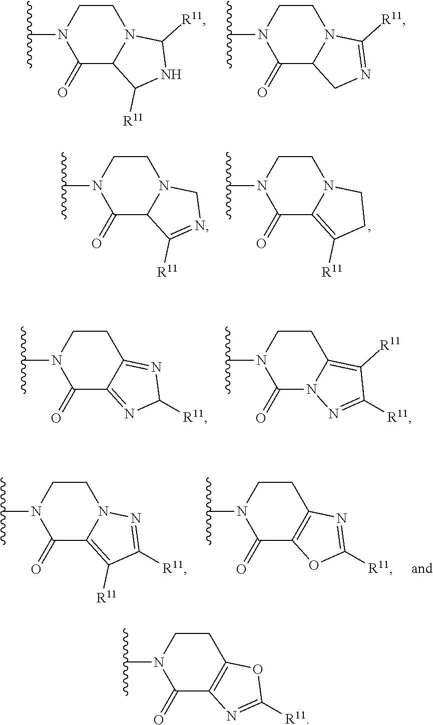

In one embodiment of Formula (I), (II), (LP-I), (LP-II), (CONJ-I), or (CONJ-II), including any of the foregoing, R^{1b} is hydrogen or C₁₋₆ alkyl; R^{2b} is a 3- to 12-membered heterocycle containing at least one N atom and the heterocycle is further substituted with at least one R⁵³; and R^{1a} and R^{2a} are joined together with the atoms to which they are attached to form an optionally substituted N—C(O)-linked 3- to 12-membered fused heterocycle of selected from:

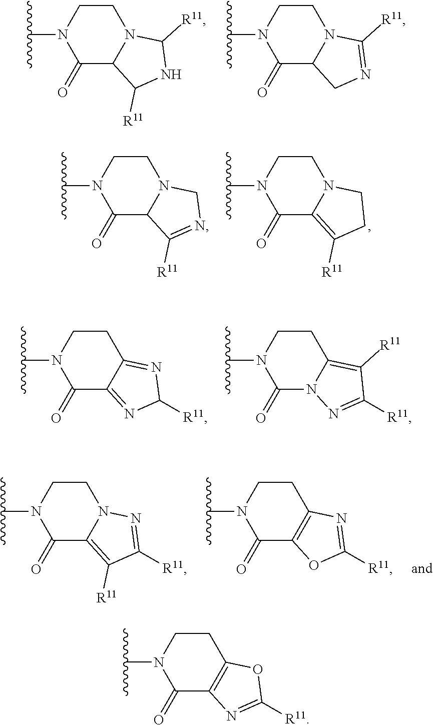

In one embodiment of Formula (I), (II), (LP-I), (LP-II), (CONJ-I), or (CONJ-II), including any of the foregoing, R^{1a} and R^{2a} are joined together with the atoms to which they are attached to form an optionally substituted N—C(O)-linked 3- to 12-membered fused heterocycle and R^{1b} and R^{2b} are joined together with the atoms to which they are attached to form an optionally substituted N—C(O)-linked 3- to 12-membered fused heterocycle wherein the optionally substituted N—C(O)-linked 3- to 12-membered fused heterocycle is independently selected from:

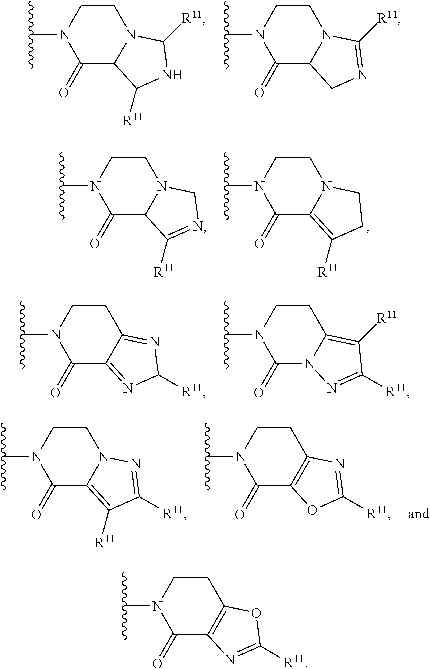

In one embodiment of Formula (I), (II), (LP-I), (LP-II), (CONJ-I), or (CONJ-II), including any of the foregoing, R^{1a} and R^{2a} are joined together with the atoms to which they are attached to form

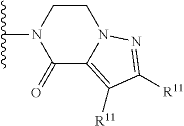

and R^{1b} and R^{2b} are joined together with the atoms to which they are attached to form

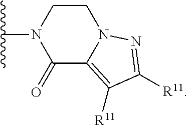

In one embodiment of Formula (I), (II), (LP-I), (LP-II), (CONJ-I), or (CONJ-II), including any of the foregoing, R^{1a} and R^{2a} are joined together with the atoms to which they are attached to form

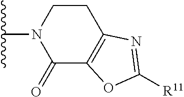

and R^{1b} and R^{2b} are joined together with the atoms to which they are attached to form

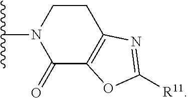

In one embodiment of Formula (I), (II), (LP-I), (LP-II), (CONJ-I), or (CONJ-II), including any of the foregoing, R^{1b} and R^{2b} are joined together with the atoms to which they are attached to form

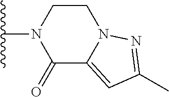 [SMILES: *N1CCn2nc(C)cc2C1=O]

and R^{1a} and R^{2a} are joined together with the atoms to which they are attached to form

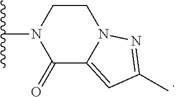 [SMILES: *N1CCn2nc(C)cc2C1=O]

In one embodiment of Formula (I), (II), (LP-I), (LP-II), (CONJ-I), or (CONJ-II) including any of the foregoing, R^{1b} and R^{2b} are joined together with the atoms to which they are attached to form

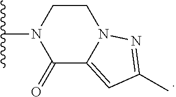 [SMILES: *N1CCn2nc(C)cc2C1=O]

and R^{1a} and R^{2a} are joined together with the atoms to which they are attached to form

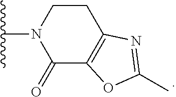 [SMILES: *N1CCc2nc(C)oc2C1=O]

In one embodiment of Formula (III), (LP-III), or (CONJ-III), including any of the foregoing, R^{9a} and R^{10a} are joined together with the atoms to which they are attached to form

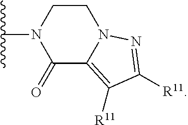

and R^{9b} and R^{10b} are joined together with the atoms to which they are attached to form

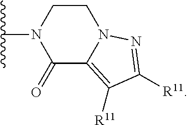

In one embodiment of Formula (III), (LP-III), or (CONJ-III), including any of the foregoing, R^{9a} and R^{9a} are joined together with the atoms to which they are attached to form

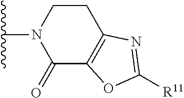

and R^{9b} and R^{10b} are joined together with the atoms to which they are attached to form

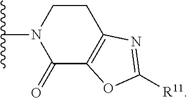

In one embodiment of Formula (III), (LP-III), or (CONJ-III), including any of the foregoing, R^{9b} and R^{10b} are joined together with the atoms to which they are attached to form

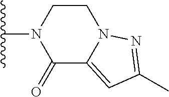 [SMILES: *N1CCn2nc(C)cc2C1=O]

and R^{9a} and R^{10a} are joined together with the atoms to which they are attached to form

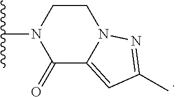 [SMILES: *N1CCn2nc(C)cc2C1=O]

In one embodiment of Formula (III), (LP-III), or (CONJ-III), including any of the foregoing, R^{9b} and R^{10b} are joined together with the atoms to which they are attached to form

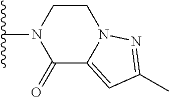 [SMILES: *N1CCn2nc(C)cc2C1=O]

and R^{9a} and R^{10a} are joined together with the atoms to which they are attached to form

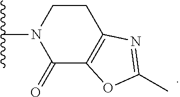 [SMILES: *N1CCc2nc(C)oc2C1=O]

In one embodiment of Formula (I), (II), (LP-I), (LP-II), (CONJ-I), or (CONJ-II), including any of the foregoing, R^{1a} is hydrogen; R^{2a} is

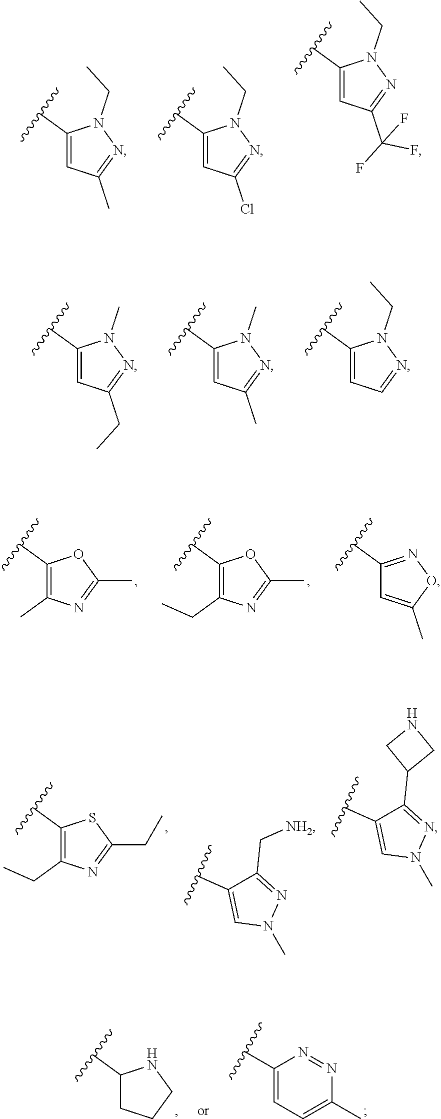

and R^{1b} and R^{2b} are joined together with the atoms to which they are attached to form

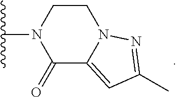 [SMILES: *N1CCn2nc(C)cc2C1=O]

In one embodiment of Formula (I), (II), (LP-I), (LP-II), (CONJ-I), or (CONJ-II), including any of the foregoing, R^{1b} is hydrogen; R^{2b} is

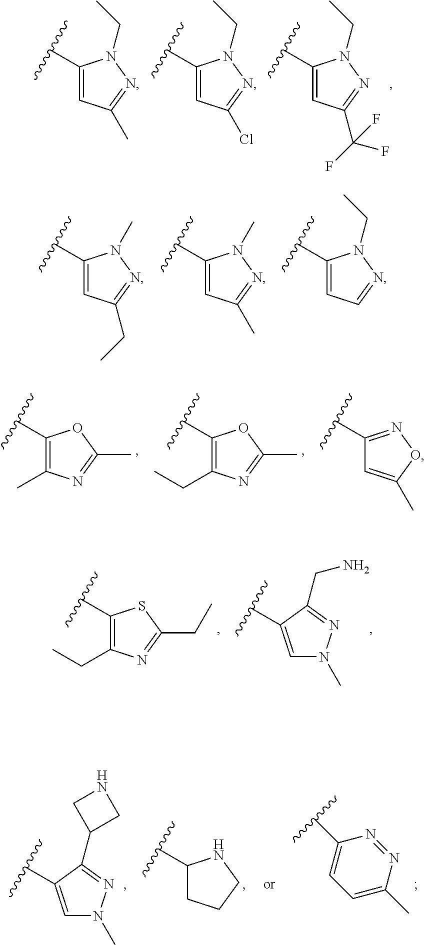

and R^{1a} and R^{2a} are joined together with the atoms to which they are attached to form

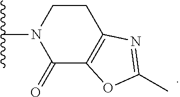 [SMILES: *N1CCc2nc(C)oc2C1=O]

In one embodiment of Formula (I), (II), (LP-I), (LP-II), (CONJ-I), or (CONJ-II), including any of the foregoing, R^{1a} is hydrogen; R^{2a} is

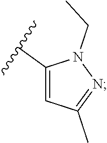 [SMILES: *c1cc(C)nn1CC]

and R^{1b} and R^{2b} are joined together with the atoms to which they are attached to form

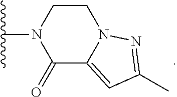 [SMILES: *N1CCn2nc(C)cc2C1=O]

In one embodiment of Formula (I), (II), (LP-I), (LP-II), (CONJ-I), or (CONJ-II), including any of the foregoing, R^{1b} is hydrogen; R^{2b} is

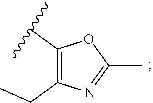 [SMILES: *c1oc(C)nc1CC]

and R^{1a} and R^{2a} are joined together with the atoms to which they are attached to form

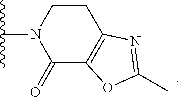 [SMILES: *N1CCc2nc(C)oc2C1=O]

In one embodiment of Formula (I), (II), (LP-I), (LP-II), (CONJ-I), or (CONJ-II), including any of the foregoing, R^{1b} is hydrogen; R^{2b} is

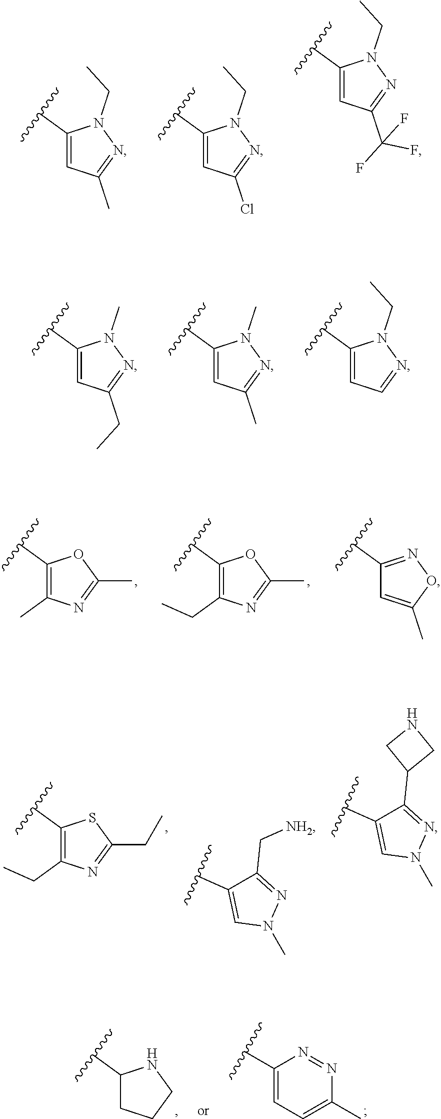

and R^{1a} and R^{2a} are joined together with the atoms to which they are attached to form

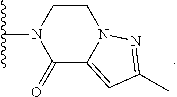 [SMILES: *N1CCn2nc(C)cc2C1=O]

In one embodiment of Formula (I), (II), (LP-I), (LP-II), (CONJ-I), or (CONJ-II), including any of the foregoing, R^{1b} is hydrogen; R^{2b} is

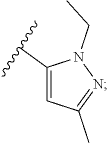 [SMILES: *c1cc(C)nn1CC]

and R^{1a} and R^{2a} are joined together with the atoms to which they are attached to form

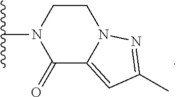 [SMILES: *N1CCn2nc(C)cc2C1=O]

In one embodiment of Formula (I), (II), (LP-I), (LP-II), (LP-IV), (LP-V), (CONJ-I), (CONJ-II), (CONJ-IV), (CONJ-V), including any of the foregoing, R^{2a} is a C₃₋₁₂ carbocycle optionally substituted with one or more R⁵³. In one embodiment of Formula (I), (II), (LP-I), (LP-II), (LP-IV), (LP-V), (CONJ-I), (CONJ-II), (CONJ-IV), (CONJ-V), including any of the foregoing, R^{2a} is a C₈₋₁₂ carbocycle optionally substituted with one or more R⁵³. In one embodiment of Formula (I), (II), (LP-I), (LP-II), (LP-IV), (CONJ-I), (CONJ-II), or (CONJ-IV), including any of the foregoing, R^{2b} is a C₃₋₁₂ carbocycle optionally substituted with one or more R⁵³. In one embodiment of Formula (I), (II), (LP-I), (LP-II), (LP-IV), (CONJ-I), (CONJ-II), or (CONJ-IV), including any of the foregoing, R^{2b} is a C₈₋₁₂ carbocycle optionally substituted with one or more R⁵³. In one embodiment of Formula (I), (II), (LP-I), (LP-II), (LP-IV), (LP-V), (CONJ-I), (CONJ-II), (CONJ-IV), (CONJ-V), including any of the foregoing, the C₃₋₁₂ carbocycle is an optionally substituted bicyclic C₃₋₁₂ carbocycle, and can be bridged, fused, or spirocyclic. In one embodiment of Formula (I), (II), (LP-I), (LP-II), (LP-IV), (LP-V), (CONJ-I), (CONJ-II), (CONJ-IV), (CONJ-V), including any of the foregoing, the C₈₋₁₂ carbocycle is an optionally substituted bicyclic C₃₋₁₂ carbocycle, and can be bridged, fused, or spirocyclic. In one embodiment of Formula (I), (II), (LP-I), (LP-II), (LP-IV), (LP-V), (CONJ-I), (CONJ-II), (CONJ-IV), (CONJ-V), including any of the foregoing, R^{2a} and/or R^{2b} is an optionally substituted bridged C₃₋₁₂ carbocycle substituted with one R⁵³ and R⁵³ is C₁₋₆ alkyl. In one embodiment of Formula (I), (II), (LP-I), (LP-II), (LP-IV), (LP-V), (CONJ-I), (CONJ-II), (CONJ-IV), (CONJ-V), including any of the foregoing, R^{2a} and/or R^{2b} is an optionally substituted bridged C₈₋₁₂ carbocycle substituted with one R⁵³ and R⁵³ is C₁₋₆ alkyl. In certain embodiments, R⁵³ is an amino acid residue.

In one embodiment of Formula (I), (II), (LP-I), (LP-II), (LP-IV), (LP-V), (CONJ-I), (CONJ-II), (CONJ-IV), (CONJ-V), including any of the foregoing, R^{2a} is selected from

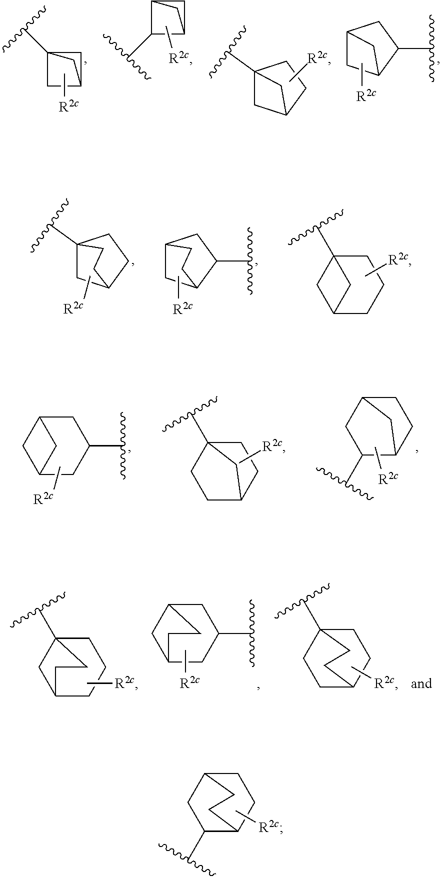

wherein R^{2c} is hydrogen, C₁₋₆ alkyl, halo, C₁₋₆ alkyl optionally substituted with one or more R⁵⁴, or 3- to 12-membered heterocycle.

In one embodiment of Formula (I), (II), (LP-I), (LP-II), (LP-IV), (CONJ-I), (CONJ-II), or (CONJ-IV), including any of the foregoing, R^{2b} is selected from

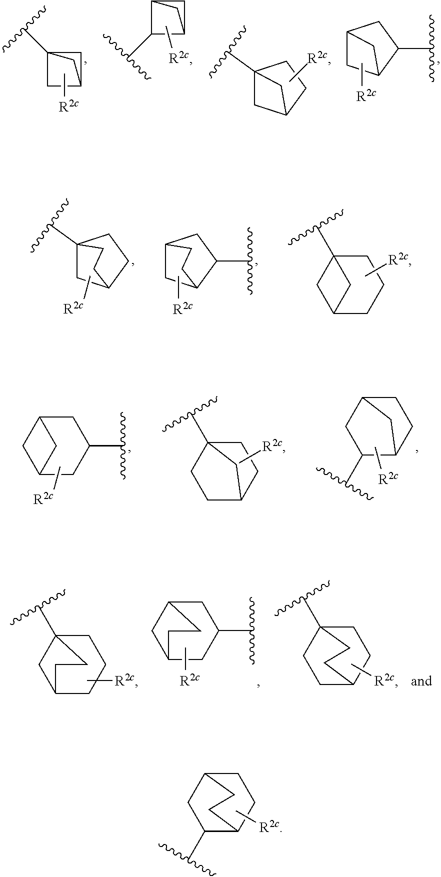

In one embodiment of Formula (I), (II), (LP-I), (LP-II), (LP-IV), (LP-V), (CONJ-I), (CONJ-II), (CONJ-IV), (CONJ-V), including any of the foregoing, R^{2a} is

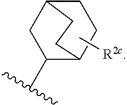

In one embodiment of Formula (I), (II), (LP-I), (LP-II), (LP-IV), (CONJ-I), (CONJ-II), or (CONJ-IV), including any of the foregoing, R^{2b} is

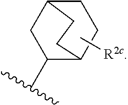

In one embodiment of Formula (I), (II), (LP-I), (LP-II), (LP-IV), (LP-V), (CONJ-I), (CONJ-II), (CONJ-IV), (CONJ-V), including any of the foregoing, R^{2a} is

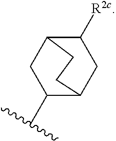

In one embodiment of Formula (I), (II), (LP-I), (LP-II), (LP-IV), (CONJ-I), (CONJ-II), or (CONJ-IV), including any of the foregoing, R^{2b} is

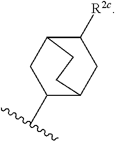

In one embodiment of Formula (I), (II), (LP-I), (LP-II), (LP-IV), (LP-V), (CONJ-I), (CONJ-II), (CONJ-IV), (CONJ-V), including any of the foregoing, R^{2a} is

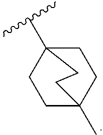 [SMILES: *C12CCC(C)(CC1)CC2]

In one embodiment of Formula (I), (II), (LP-I), (LP-II), (LP-IV), (CONJ-I), (CONJ-II), or (CONJ-IV), including any of the foregoing, R^{2b} is

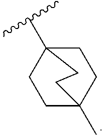 [SMILES: *C12CCC(C)(CC1)CC2]

In one embodiment of Formula (I), (II), (LP-I), (LP-II), (LP-IV), (CONJ-I), (CONJ-II), or (CONJ-IV), including any of the foregoing, R^{2a} is selected from

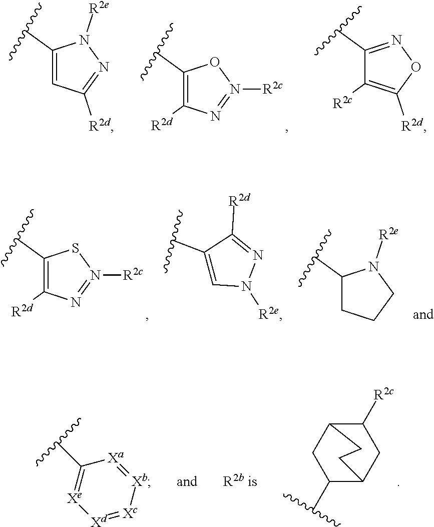

In one embodiment of Formula (I), (II), (LP-I), (LP-II), (LP-IV), (CONJ-I), (CONJ-II), or (CONJ-IV), including any of the foregoing, R^{2a} is selected from

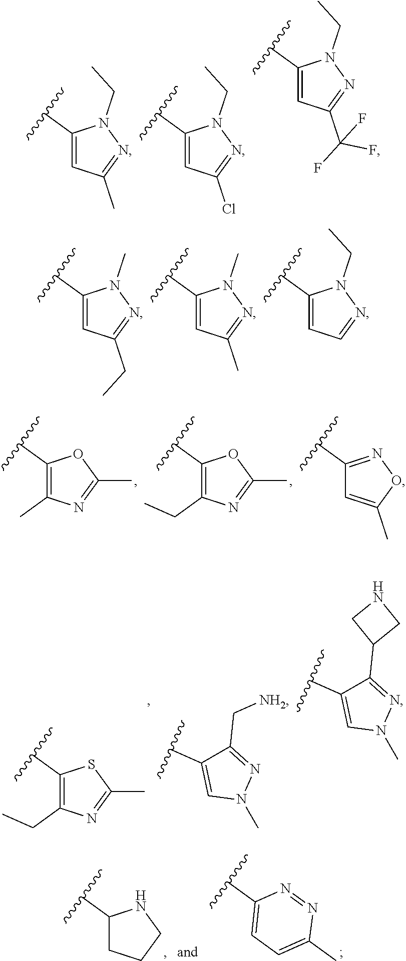

and R^{2b} is an optionally substituted C₈₋₁₂ carbocycle. In one embodiment of Formula (I), (II), (LP-I), (LP-II), (LP-IV), (CONJ-I), (CONJ-II), or (CONJ-IV), including any of the foregoing, R^{2a} is selected from

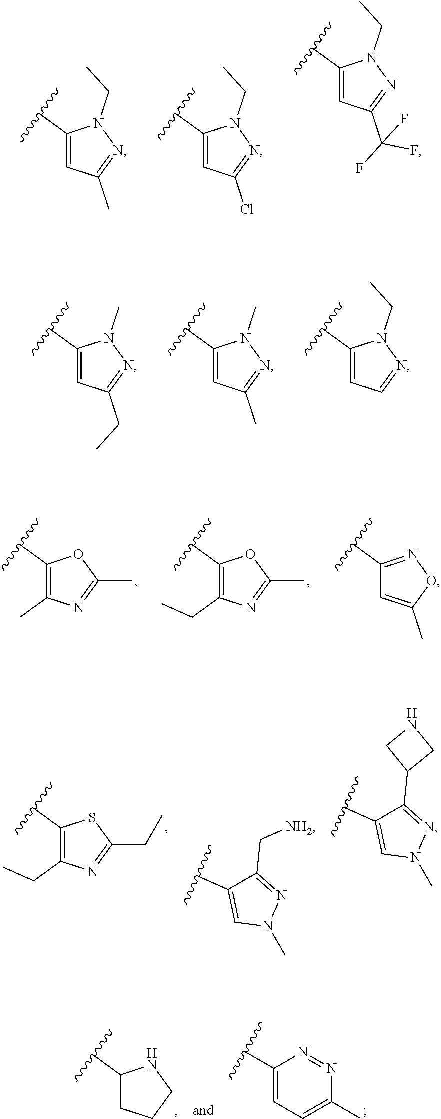

### and R^{2b} is

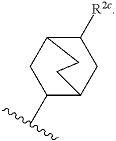

In one embodiment of Formula (I), (II), (LP-I), (LP-II), (LP-IV), (CONJ-I), (CONJ-II), or (CONJ-IV), including any of the foregoing, R^{2a} is

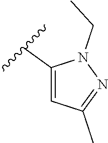 [SMILES: *c1cc(C)nn1CC]

### and R^{2b} is

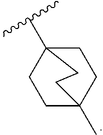 [SMILES: *C12CCC(C)(CC1)CC2]

In one embodiment of Formula (I), (II), (LP-I), (LP-II), (CONJ-I), or (CONJ-II), including any of the foregoing, R² is

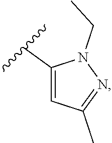 [SMILES: *c1cc(C)nn1CC]

### R² is

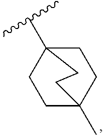 [SMILES: *C12CCC(C)(CC1)CC2]

and R^{1a} and R^{1b} are both hydrogen.

In one embodiment of Formula (I)—(IV), (LP-I)-(LPV), or (CONJ-I)—(CONJ-IV), including any of the foregoing, L¹ is —C₂₋₁₀alkenyl- optionally substituted with one or more R⁵⁰. In one embodiment of Formula (I)—(IV), (LP-I)-(LPV), or (CONJ-I)—(CONJ-IV), including any of the foregoing, L¹ is —CH═CH— optionally substituted with one R⁵⁰. In one embodiment of Formula (I)—(IV), (LP-I)-(LPV), or (CONJ-I)—(CONJ-IV), including any of the foregoing, L¹ is —CH═CH—.

In one embodiment of Formula (I)—(IV), (LP-I)-(LPV), or (CONJ-I)—(CONJ-IV), including any of the foregoing, L¹ is —C₁₋₁₀alkyl- optionally substituted with one or more R⁵⁰. In one embodiment of Formula (I)—(IV), (LP-I)-(LPV), or (CONJ-I)—(CONJ-IV), including any of the foregoing, L¹ is —CH₂—CH₂— optionally substituted with one R⁵⁰. In one embodiment of Formula (I)—(IV), (LP-I)-(LPV), or (CONJ-I)—(CONJ-IV), including any of the foregoing, L¹ is —CH₂—CH₂—.

In one embodiment of Formula (I)—(IV), (LP-I)-(LPV), or (CONJ-I)—(CONJ-IV), including any of the foregoing, L¹ is

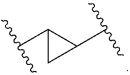 [SMILES: *C1CC1*]

In one embodiment of Formula (I), (III), (IV), (LP-I), (LP-III), (LP-IV), (CONJ-I), (CONJ-III), or (CONJ-IV) including any of the foregoing, L² is C₁₋₆ alkyl. In one embodiment of Formula (I), (III), (IV), (LP-I), (LP-III), (LP-IV), (CONJ-I), (CONJ-III), or (CONJ-IV) including any of the foregoing, L² is C₃₋₆ alkyl. In one embodiment of Formula (I), (III), (IV), (LP-I), (LP-III), (LP-IV), (CONJ-I), (CONJ-III), or (CONJ-IV) including any of the foregoing, L² is —(CH₂)₃—. In one embodiment of Formula (I), (III), (IV), (LP-I), (LP-III), (LP-IV), (CONJ-I), (CONJ-III), or (CONJ-IV) including any of the foregoing, L² is C₁₋₆ alkyl optionally substituted with one R⁵⁰.

In one embodiment of Formula (I), (II), (LP-I), (LP-II), (CONJ-I), or (CONJ-II), including any of the foregoing, L¹ is —CH═CH—; L² is —(CH₂)₃—; R²⁰ is —CONH₂; R^{1a} is hydrogen; R^{2a} is

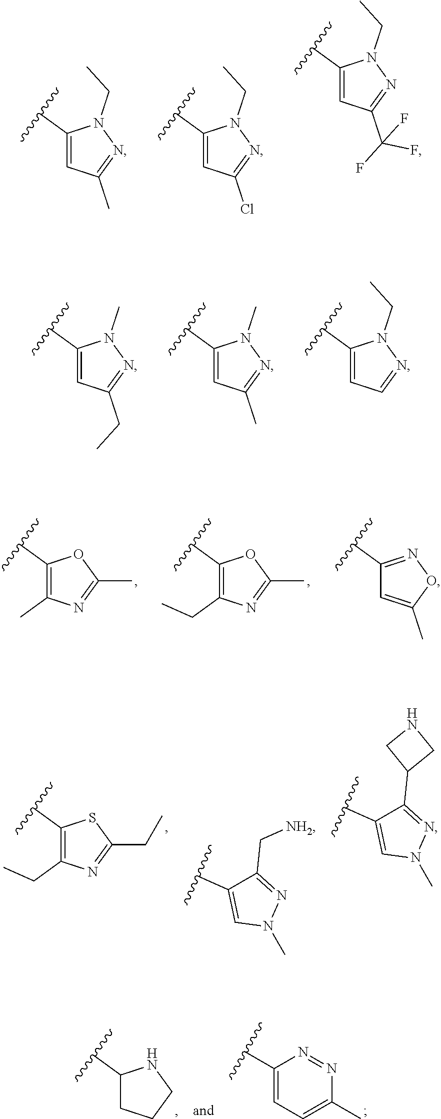

and R^{1b} and R^{2b} are joined together with the atoms to which they are attached to form

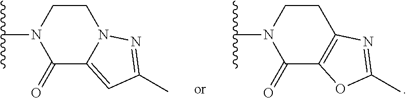 [SMILES: *N1CCn2nc(C)cc2C1=O ; *N1CCc2nc(C)oc2C1=O]

In one embodiment of Formula (I), (II), (LP-I), (LP-II), (CONJ-I), or (CONJ-II), including any of the foregoing, L¹ is —CH═CH—; L² is —(CH₂)₃—; R²⁰ is —CONH₂; R^{1b} is hydrogen; R^{2b} is

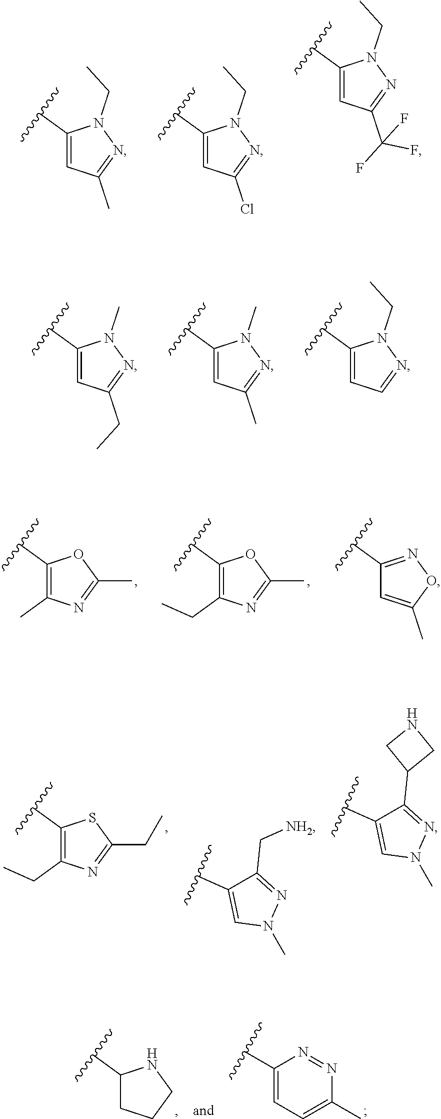

and R^{1a} and R^{2a} are joined together with the atoms to which they are attached to form

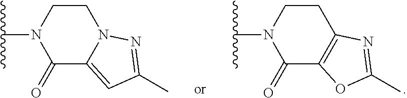 [SMILES: *N1CCn2nc(C)cc2C1=O ; *N1CCc2nc(C)oc2C1=O]

In one embodiment of Formula (I), (II), (LP-I), (LP-II), (CONJ-I), or (CONJ-II), including any of the foregoing, L¹ is —CH═CH—; L² is —(CH₂)₃—; R²⁰ is —CONH₂; R^{2a} and R^{2b}are independently selected from

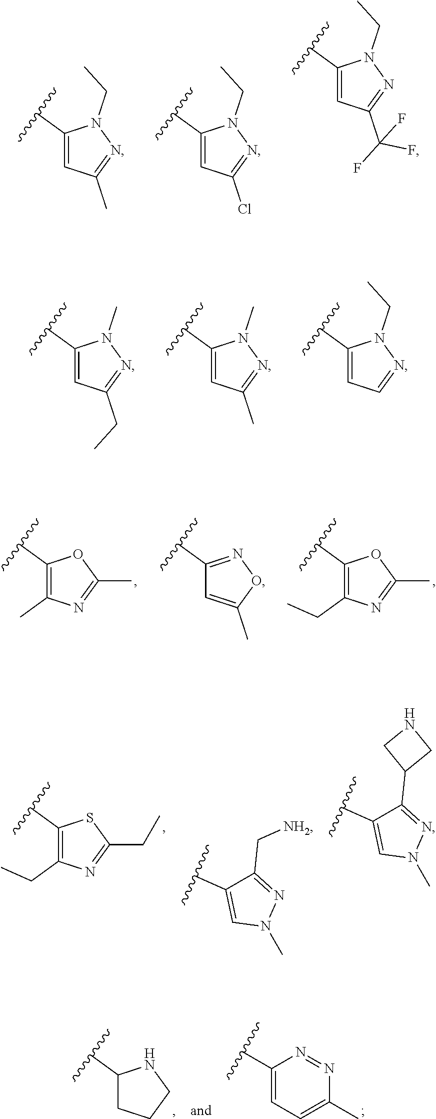

and R^{1a} and R^{1b} are both hydrogen.

In one embodiment of Formula (I), (II), (LP-I), (LP-II), (CONJ-I), or (CONJ-II), including any of the foregoing, L¹ is —CH₂—CH₂—; L² is —(CH₂)₃—; R²⁰ is —CONH₂; R^{2a} and R^{2b}are independently selected from

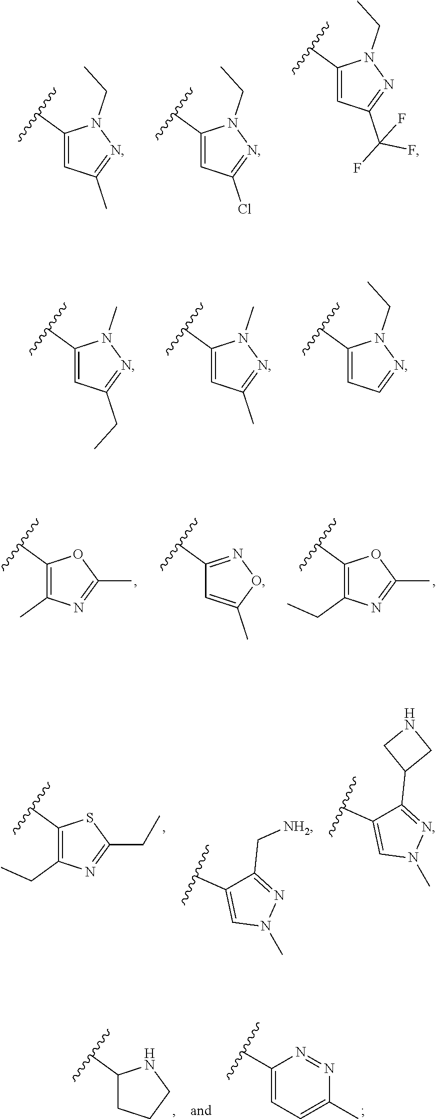

and R^{1a} and R^{1b} are both hydrogen.

In one embodiment of Formula (I), (II), (LP-I), (LP-II), (CONJ-I), or (CONJ-II), including any of the foregoing, L¹ is —CH═CH—; L² is —(CH₂)₃—; R²⁰ is hydrogen; R^{2a} and R^{2b}are independently selected from

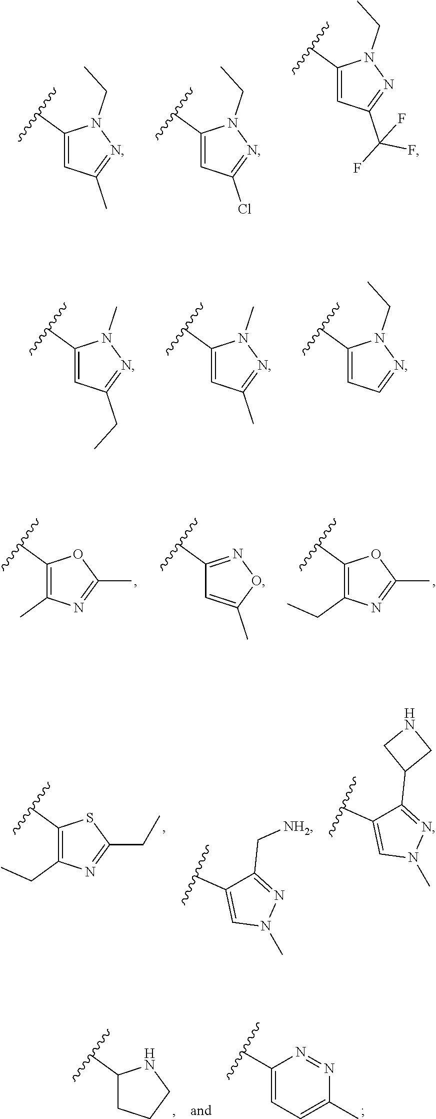

and R^{1a} and R^{1b} are both hydrogen.

In one embodiment of Formula (I), (II), (LP-I), (LP-II), (CONJ-I), or (CONJ-II), including any of the foregoing, L¹ is —CH═CH—; L² is —(CH₂)₃—; R²⁰ is —CONH₂; R^{1a} and R^{1b} are both hydrogen; R^{2a} is

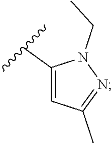 [SMILES: *c1cc(C)nn1CC]

and R^{2b} is selected from

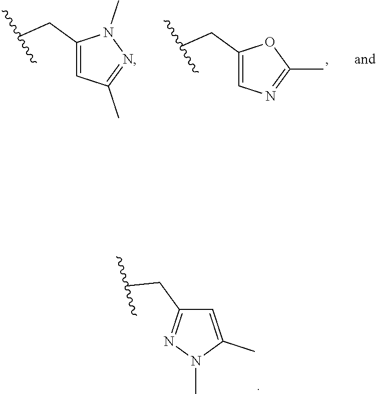

In one embodiment of Formula (I), (II), (LP-I), (LP-II), (CONJ-I), or (CONJ-II), including any of the foregoing, L¹ is —CH═CH—; L² is —(CH₂)₃—; R²⁰ is —CONH₂; R^{1a} and R^{1b}are both hydrogen; R^{2a} is

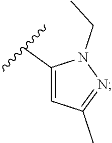 [SMILES: *c1cc(C)nn1CC]

### and R^{2b} is

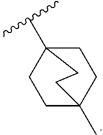 [SMILES: *C12CCC(C)(CC1)CC2]

In one embodiment of Formula (I), (II), (LP-I), (LP-II), (CONJ-I), or (CONJ-II), including any of the foregoing, L¹ is —CH═CH—; L² is —(CH₂)₃—; R²⁰ is —CONH₂; R^{1a} and R^{2a} are joined together with the atoms to which they are attached to form

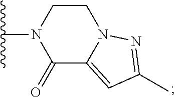 [SMILES: *N1CCn2nc(C)cc2C1=O]

and R^{1b} and R^{2b} are joined together with the atoms to which they are attached to form

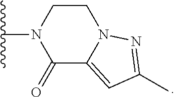 [SMILES: *N1CCn2nc(C)cc2C1=O]

In one embodiment of Formula (I), (II), (LP-I), (LP-II), (CONJ-I), or (CONJ-II), including any of the foregoing, L¹ is —CH═CH—; L² is —(CH₂)₃—; R²⁰ is —CONH₂; R^{1a} and R^{2a} are joined together with the atoms to which they are attached to form

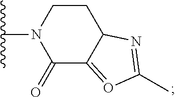

and R^{1b} and R^{2b} are joined together with the atoms to which they are attached to form

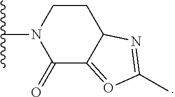

In one embodiment of Formula (III), (LP-III), or (CONJ-III), including any of the foregoing, L¹ is —CH═CH—; L² is —(CH₂)₃—; R²⁰ is —CONH₂; R^{9b} and R^{10b} are joined together with the atoms to which they are attached to form

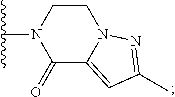 [SMILES: *N1CCn2nc(C)cc2C1=O]

and R^{9b} and R^{9b} are joined together with the atoms to which they are attached to form

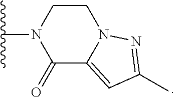 [SMILES: *N1CCn2nc(C)cc2C1=O]

In one embodiment of Formula (IV), (LP-IV), or (CONJ-IV), including any of the foregoing, L¹ is —CH═CH—; L² is —(CH₂)₃—; R²⁰ is —CONH₂; R^{2a} and R^{2b} are independently selected from

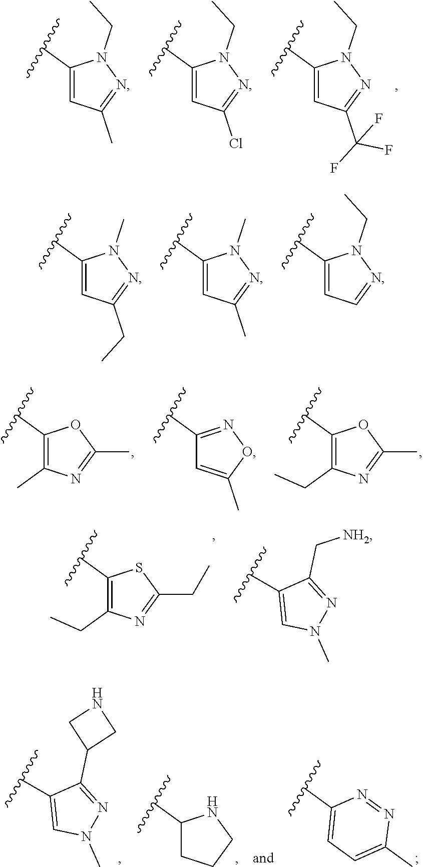

and R^{1a} is hydrogen.

In one embodiment of Formula (I), (III), (LP-I), (LP-III)-(LP-V), or (CONJ-I)—(CONJ-V), including any of the foregoing, X¹ is N. In one embodiment of Formula (I), (III), (LP-I), (LP-III)-(LP-V), or (CONJ-I)—(CONJ-V), including any of the foregoing, X¹ is CR³. In one embodiment of Formula (I), (III), (LP-I), (LP-III)-(LP-V), or (CONJ-I)—(CONJ-V), including any of the foregoing, X¹ is CH. In one embodiment of Formula (I), (III), (LP-I), (LP-III)-(LP-V), or (CONJ-I)—(CONJ-V), including any of the foregoing, X¹ is CR³ and R³ is OR³⁰. In one embodiment of Formula (I), (III), (LP-I), (LP-III)-(LP-V), or (CONJ-I)—(CONJ-V), including any of the foregoing, X¹ is CR³ and R³ is OC₁₋₁₀ alkyl. In one embodiment of Formula (I), (III), (LP-I), (LP-III)-(LP-V), or (CONJ-I)—(CONJ-V), including any of the foregoing, X¹ is CR³ and R³ is OCH₃.

In one embodiment of Formula (I), (III), (LP-I), (LP-III)-(LP-V), or (CONJ-I)—(CONJ-V), including any of the foregoing, R²⁰ is —CON(R^{3a})(R^{3b}). In one embodiment of Formula (I), (III), (LP-I), (LP-III)-(LP-V), or (CONJ-I)—(CONJ-V), including any of the foregoing, R²⁰ is —CONH₂. In one embodiment of Formula (I), (III), (LP-I), (LP-III)-(LP-V), or (CONJ-I)—(CONJ-V), including any of the foregoing, R²⁰ is —CON(C₁₋₆ alkyl)₂. In one embodiment of Formula (I), (III), (LP-I), (LP-III)-(LP-V), or (CONJ-I)—(CONJ-V), including any of the foregoing, R²⁰ is —CON(CH₃)₂. In one embodiment of Formula (I), (III), (LP-I), (LP-III)-(LP-V), or (CONJ-I)—(CONJ-V), including any of the foregoing, R²⁰ is hydrogen.

a. Compounds of Formula (I), (II), (III), and (IV)

In one embodiment, the compound of Formula (I) is a compound of Formula (IA) or Formula (IB):

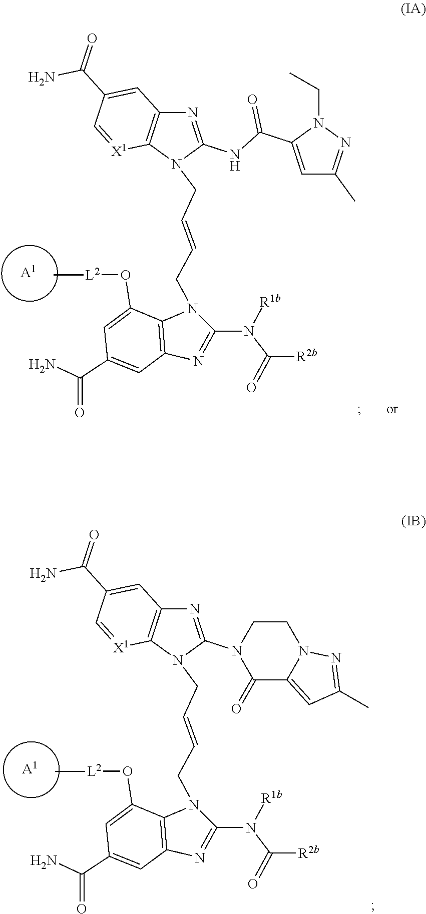

or a pharmaceutically acceptable salt or tautomer thereof.

Non-limiting examples of Formula (IA) include:

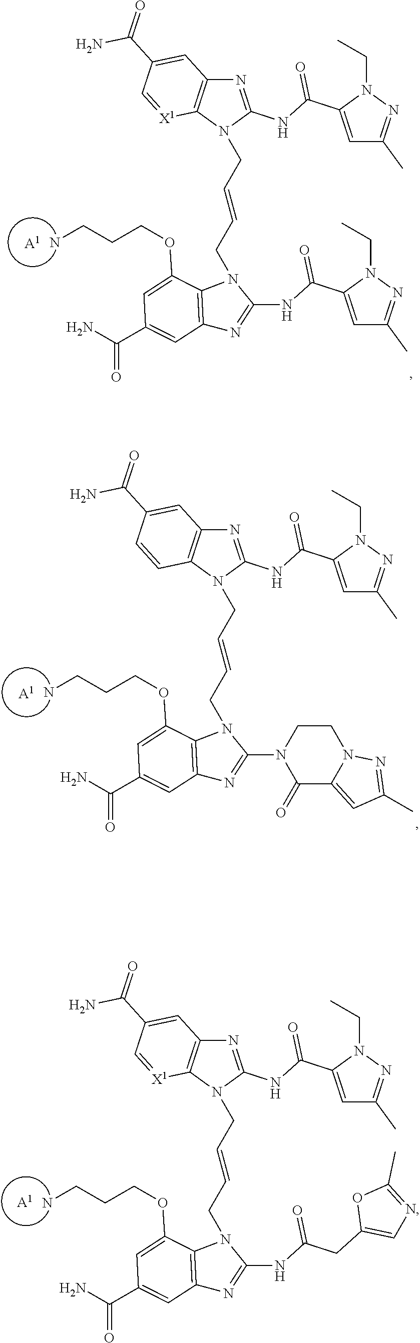

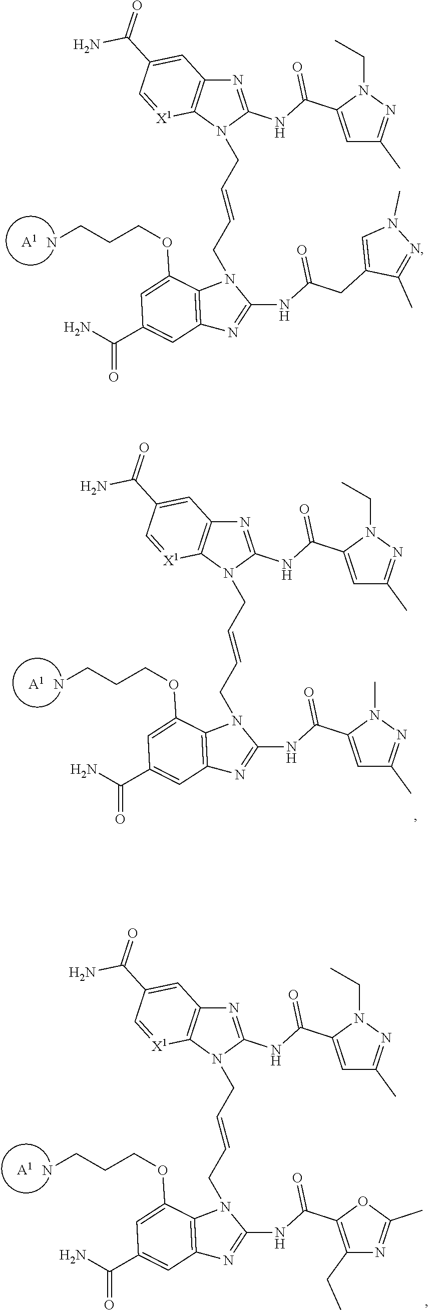

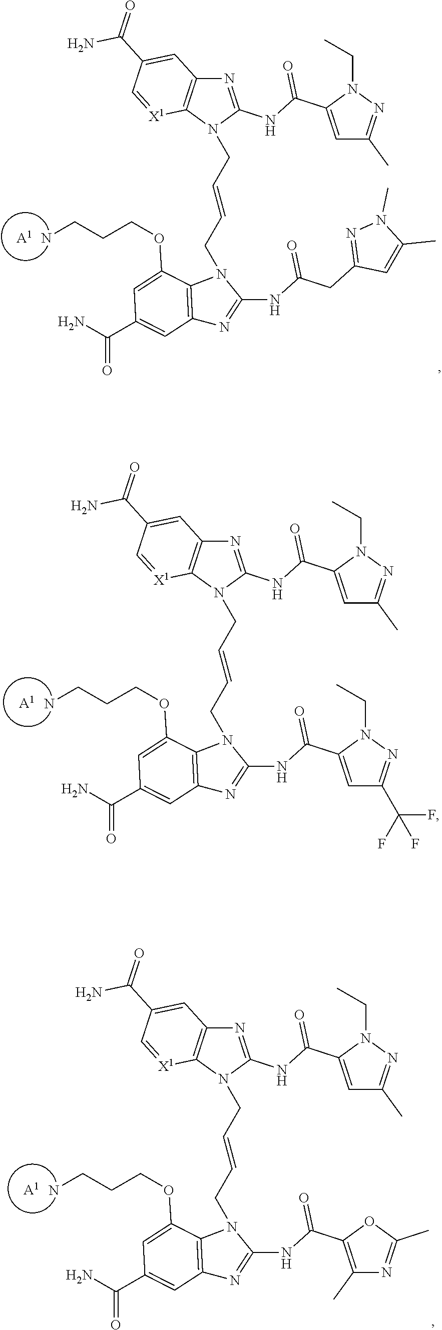

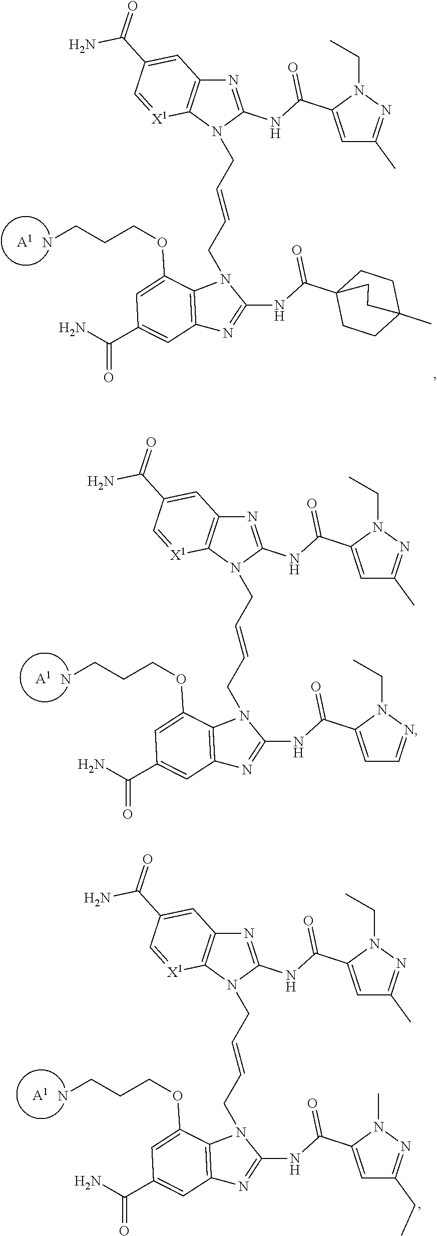

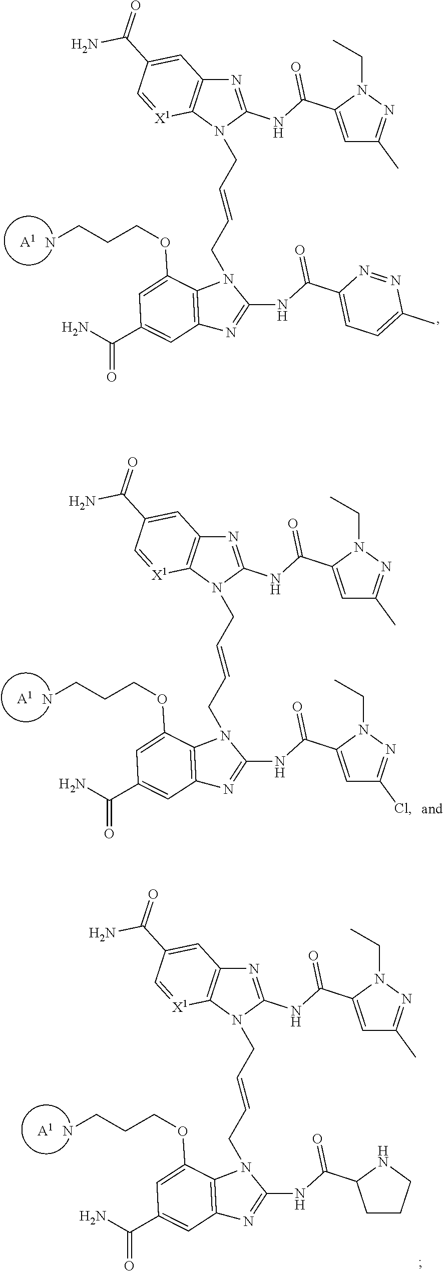

or a pharmaceutically acceptable salt or tautomer thereof.

Non-limiting examples of Formula (IB) include:

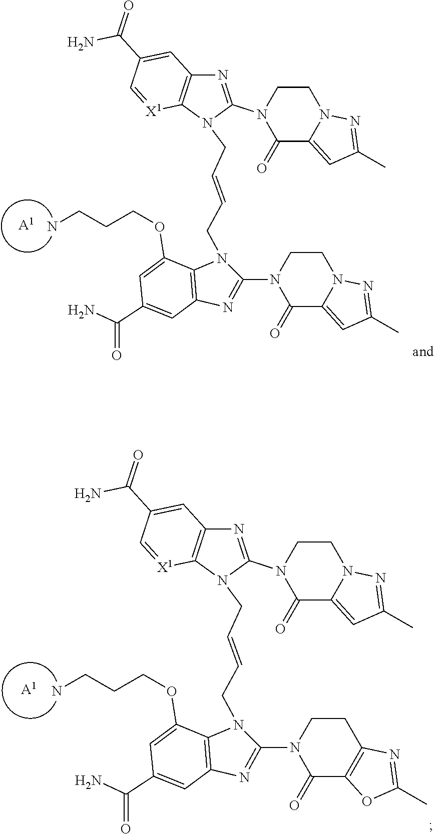

or a pharmaceutically acceptable salt or tautomer thereof.
In one embodiment, the compound of Formula (II) is a compound of Formula (IIA), Formula

(IIB), or Formula (IIC):

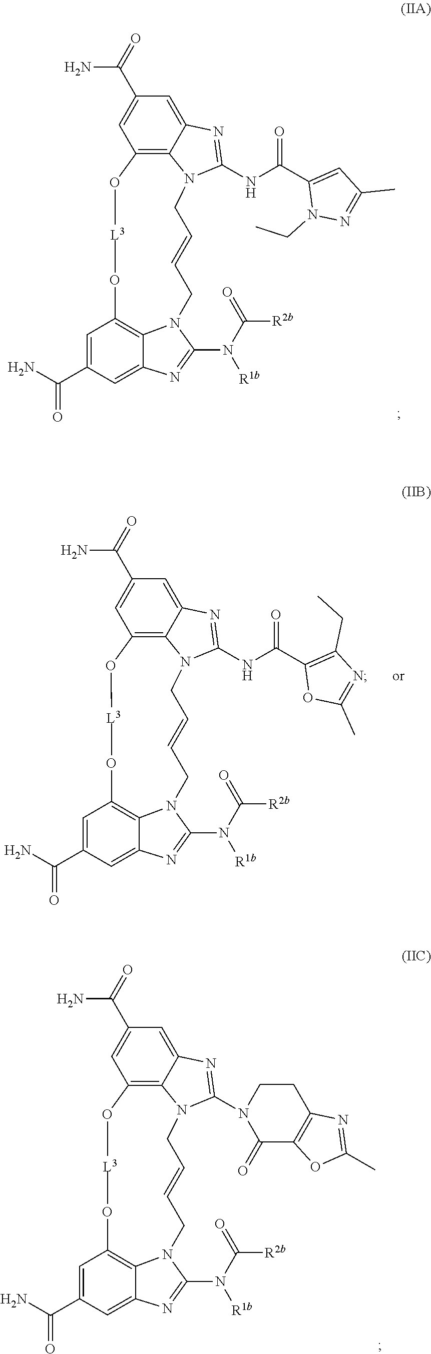

or a pharmaceutically acceptable salt or tautomer thereof.

Non-limiting examples of Formula (IIA) include:

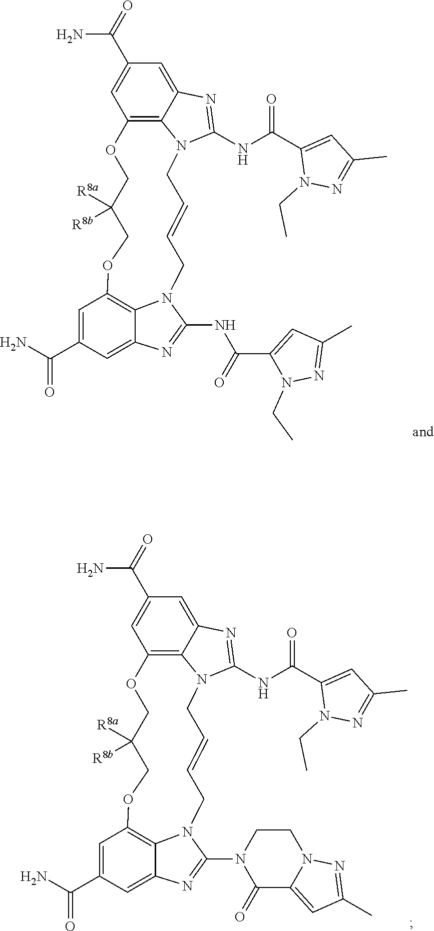

or a pharmaceutically acceptable salt or tautomer thereof.

Non-limiting examples of Formula (IIB) include:

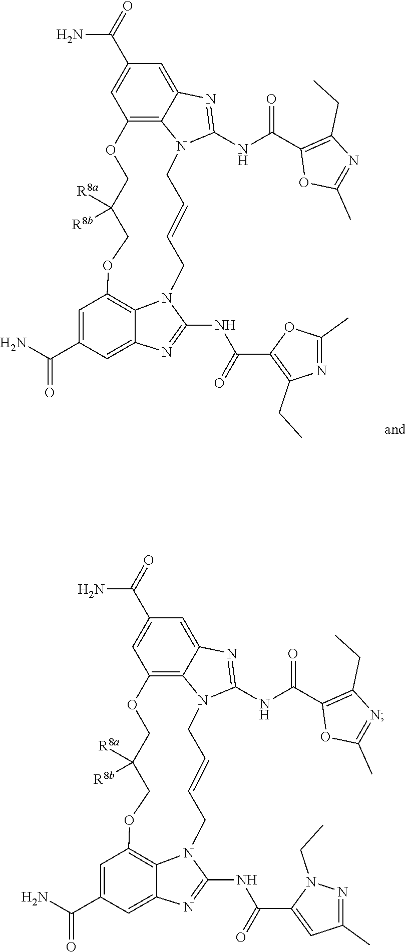

or a pharmaceutically acceptable salt or tautomer thereof.

Non-limiting examples of Formula (II) include:

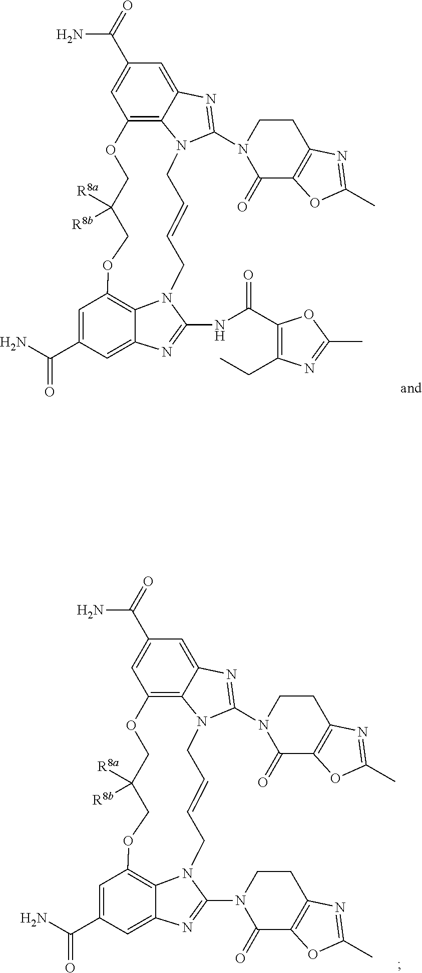

or a pharmaceutically acceptable salt or tautomer thereof.

In one embodiment, the compound of Formula (III) is a compound of Formula (IIIA):

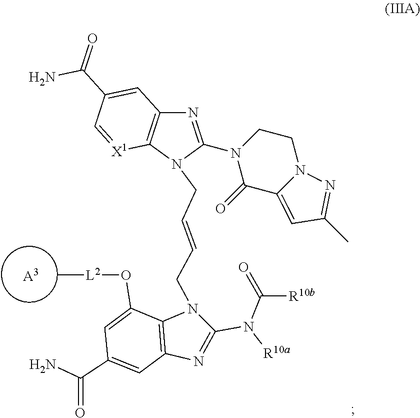

or a pharmaceutically acceptable salt or tautomer thereof.

A non-limiting example of Formula (IIIA) includes:

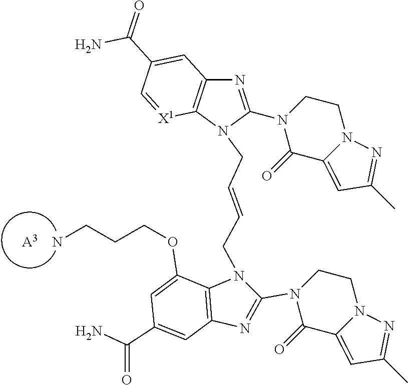

or a pharmaceutically acceptable salt or tautomer thereof.

In one embodiment, the compound of Formula (IV) is a compound of Formula (IVA):

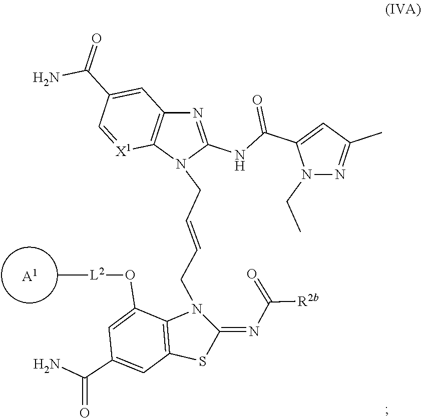

or a pharmaceutically acceptable salt or tautomer thereof.

A non-limiting example of Formula (IVA) includes:

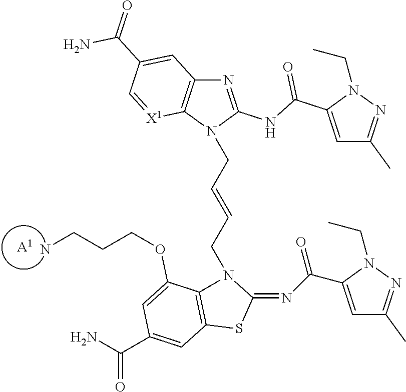

or a pharmaceutically acceptable salt or tautomer thereof.

In one embodiment of Formula (I), L¹ is —CH═CH—; R^{1a} and R^{1b} are both hydrogen; R^{2a} is

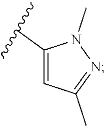 [SMILES: *c1cc(C)nn1C]

### and R^{2b} is

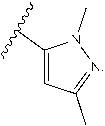 [SMILES: *c1cc(C)nn1C]

In one embodiment of Formula (I), L¹ is —CH═CH—; R^{1a} and R^{1b} are both hydrogen; R^{2a} is

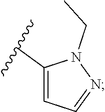 [SMILES: *c1ccnn1CC]

### and R^{2b} is

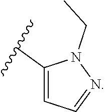 [SMILES: *c1ccnn1CC]

In one embodiment of Formula (I), L¹ is —CH═CH—; R^{1a} and R^{1b} are both hydrogen; R^{2a} is

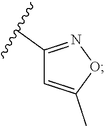 [SMILES: *c1cc(C)on1]

### and R^{2b} is

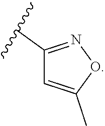 [SMILES: *c1cc(C)on1]

In one embodiment of Formula (I), L¹ is —CH═CH—; R^{1a} and R^{1b} are both hydrogen; R^{2a} is

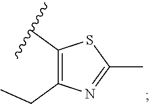 [SMILES: *c1sc(C)nc1CC]

### and R^{2b} is

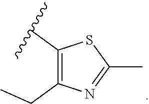 [SMILES: *c1sc(C)nc1CC]

In one embodiment of Formula (I), L¹ is —CH═CH—; R^{1a} and R^{1b} are both hydrogen; R^{2a} is

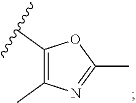 [SMILES: *c1oc(C)nc1C]

### and R^{2b} is

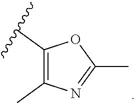 [SMILES: *c1oc(C)nc1C]

In one embodiment of Formula (I), L¹ is —CH₂CH₂—; R^{1a} and R^{1b} are both hydrogen; R^{2a} is

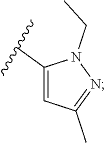 [SMILES: *c1cc(C)nn1CC]

### and R^{2b} is

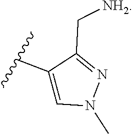 [SMILES: *c1cn(C)nc1CN]

In one embodiment of Formula (I), L¹ is —CH₂CH₂—; R^{1a} and R^{1b} are both hydrogen; R^{2a} is

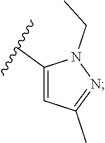 [SMILES: *c1cc(C)nn1CC]

### and R^{2b} is

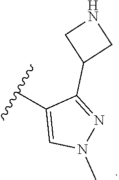 [SMILES: *c1cn(C)nc1C1CNC1]

In one embodiment of Formula (I), L¹ is —CH₂CH₂—; R^{1a} and R^{1b} are both hydrogen; R^{2a} is

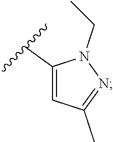 [SMILES: *c1cc(C)nn1CC]

### and R^{2b} is

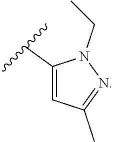 [SMILES: *c1cc(C)nn1CC]

In one embodiment of Formula (I), L¹ is —CH═CH—; R²⁰ is hydrogen; R^{1a} and R^{1b} are both hydrogen; and R^{2a} and R^{2b} are both

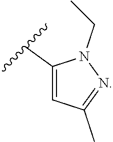 [SMILES: *c1cc(C)nn1CC]

In one embodiment of Formula (II), L¹ is —CH═CH—; R^{1a} and R^{1b} are both hydrogen and R^{2a} and R^{2b} are both

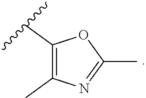 [SMILES: *c1oc(C)nc1C]

In one embodiment of Formula (I), (IA) (IB), (IV), or (IVA), including any of the foregoing, Ring A¹ is an optionally substituted bridged, fused, or spirocyclic bicyclic heterocycle comprising at least one N atom and at least one O atom wherein the heterocycle is optionally substituted with one or more R⁵³. In one embodiment of Formula (I), (IA) (IB), (IV), or (IVA), including any of the foregoing, Ring A¹ is an optionally substituted 3- to 12-membered bridged, fused, or spirocyclic bicyclic heterocycle comprising at least one nitrogen atom, including the nitrogen bound to L², and at least one oxygen atom. In one embodiment of Formula (I), (IA) (IB), (IV), or (IVA), including any of the foregoing, Ring A¹ is an optionally substituted 8- to 12-membered bridged, fused, or spirocyclic bicyclic heterocycle comprising at least one nitrogen atom, including the nitrogen bound to L², and at least one oxygen atom. In one embodiment of Formula (I), (IA) (IB), (IV), or (IVA), including any of the foregoing, Ring A¹ is an optionally substituted 3- to 12-membered N-linked bridged, fused, or spirocyclic bicyclic heterocycle comprising at least one nitrogen atom, including the nitrogen bound to L², and at least one oxygen atom. In one embodiment of Formula (I), (IA) (IB), (IV), or (IVA), including any of the foregoing, Ring A¹ is an optionally substituted 8- to 12-membered N-linked bridged, fused, or spirocyclic bicyclic heterocycle comprising at least one nitrogen atom, including the nitrogen bound to L², and at least one oxygen atom. In one embodiment of Formula (I), (IA) (IB), (IV), or (IVA), including any of the foregoing, Ring A¹ is an optionally substituted 3- to 12-membered N-linked spirocyclic bicyclic heterocycle comprising at least one nitrogen atom, including the nitrogen bound to L², and at least one oxygen atom. In one embodiment of Formula (I), (IA) (IB), (IV), or (IVA), including any of the foregoing, Ring A¹ is an optionally substituted 3- to 12-membered N-linked spirocyclic bicyclic heterocycle comprising two nitrogen atoms, including the nitrogen bound to L², and one oxygen atom.

In one embodiment of Formula (I), (IA) (IB), (IV), or (IVA), including any of the foregoing, Ring A¹ is selected from

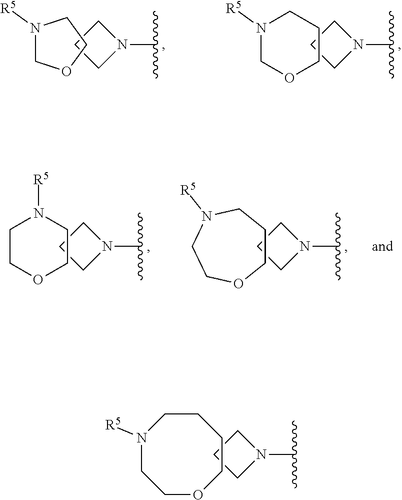

wherein R⁵ is selected from hydrogen, R⁶, —C(O)—C₁₋₆alkyl, —C(O)-heteroC₁₋₆alkyl, C₁₋₆ alkyl, and heteroC₁₋₆alkyl wherein the C₁₋₆ alkyl, either alone or part of another group, is optionally substituted with one or more R⁵⁰.

In one embodiment of Formula (I), (IA) (IB), (IV), or (IVA), including any of the foregoing, Ring A¹ is selected from

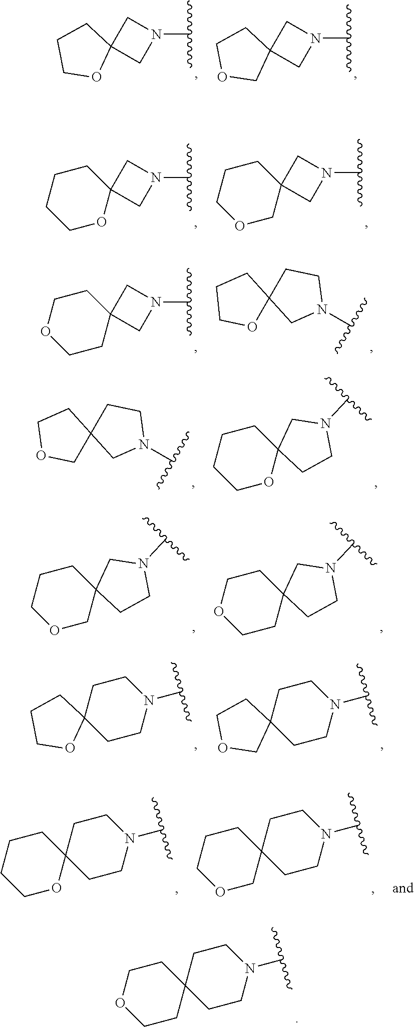

In one embodiment of Formula (I), (IA) (IB), (IV), or (IVA), including any of the foregoing, Ring A¹ is

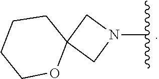 [SMILES: *N1CC2(CCCCO2)C1]

In one embodiment of Formula (I), (IA) (IB), (IV), or (IVA), including any of the foregoing, Ring A¹ is selected from

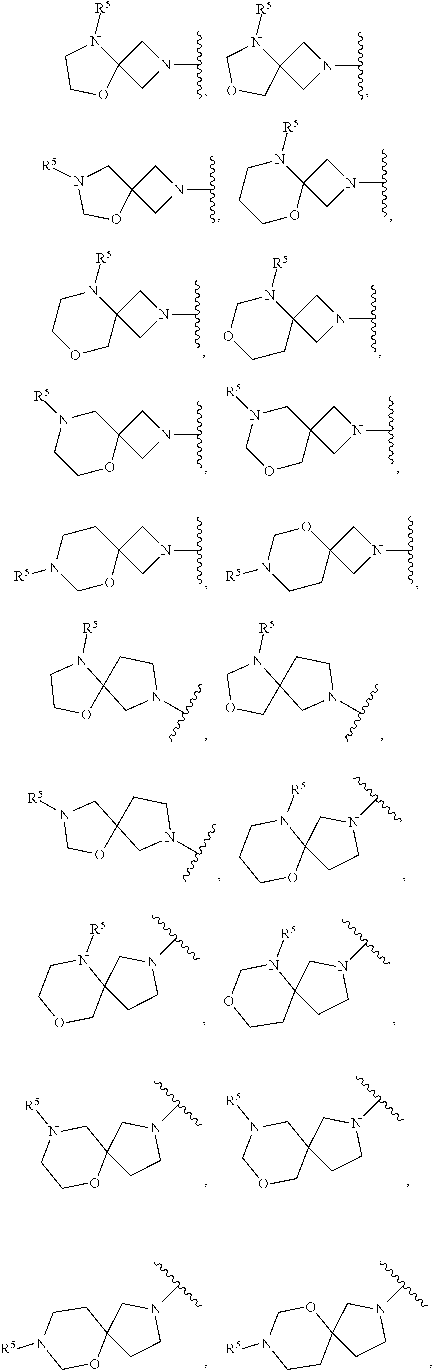

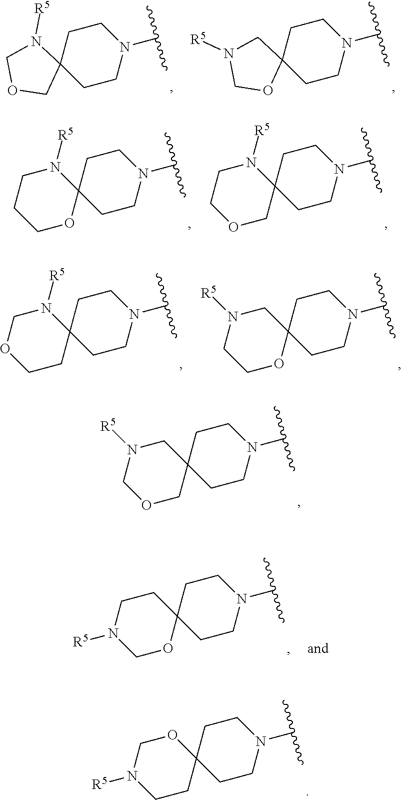

In one embodiment of Formula (I), (IA) (IB), (IV), or (IVA), including any of the foregoing, Ring A¹ is selected from

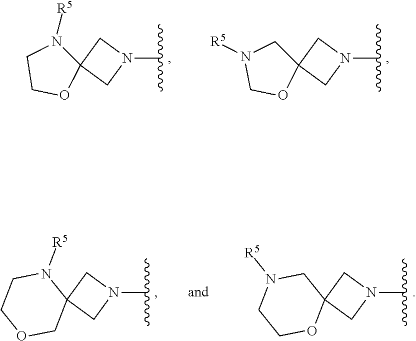

In one embodiment of Formula (I), (IA) (IB), (IV), or (IVA), including any of the foregoing, Ring A¹ is selected from

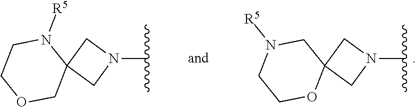

In one embodiment of Formula (I), (IA) (IB), (IV), or (IVA), including any of the foregoing, Ring A is

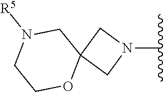

In one embodiment of Formula (I), (IA) (IB), (IV), or (IVA), including any of the foregoing, Ring A¹ is

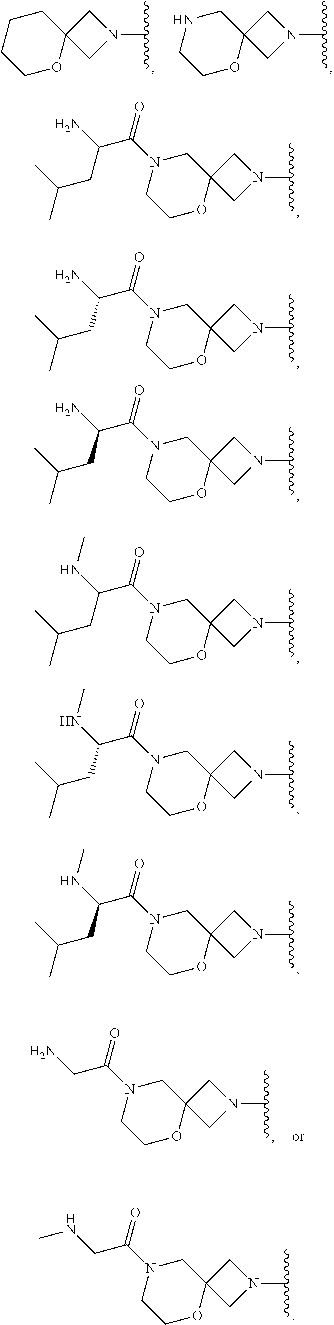

In one embodiment of Formula (I), (IA) (IB), (IV), or (IVA), including any of the foregoing, Ring A¹ is a 3- to 12-membered heterocycle substituted with R⁴. In one embodiment of Formula (I), (IA) (IB), (IV), or (IVA), including any of the foregoing, Ring A¹ is a 5- to 6-membered heterocycle substituted with R⁴.

In one embodiment of Formula (I), (IA) (IB), (IV), or (IVA), including any of the foregoing, Ring A¹ is a N-linked monocyclic 3- to 12-membered heterocycle comprising the N to which the ring is attached wherein the heterocycle is substituted with R⁴. In one embodiment of Formula (I), (IA) (IB), (IV), or (IVA), including any of the foregoing, Ring A¹ is a N-linked monocyclic 3- to 12-membered heterocycle comprising the N to which the ring is attached and a second NH wherein the heterocycle is substituted with R⁴.

In one embodiment of Formula (I), (IA) (IB), (IV), or (IVA), including any of the foregoing, Ring A¹ is selected from

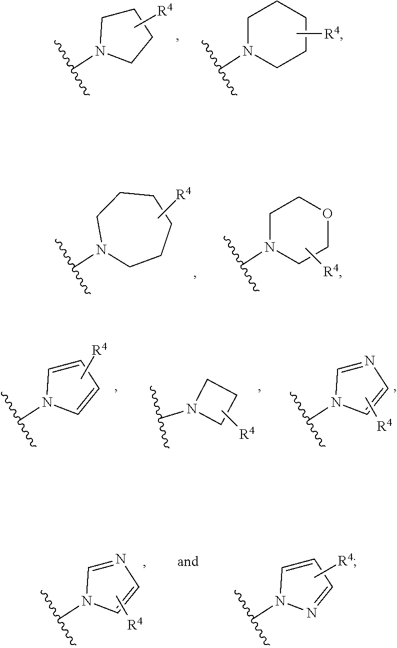

wherein

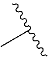

is the point of attachment to the rest of the compound.

In one embodiment of Formula (I), (IA) (IB), (IV), or (IVA), including any of the foregoing, Ring A¹ is selected from

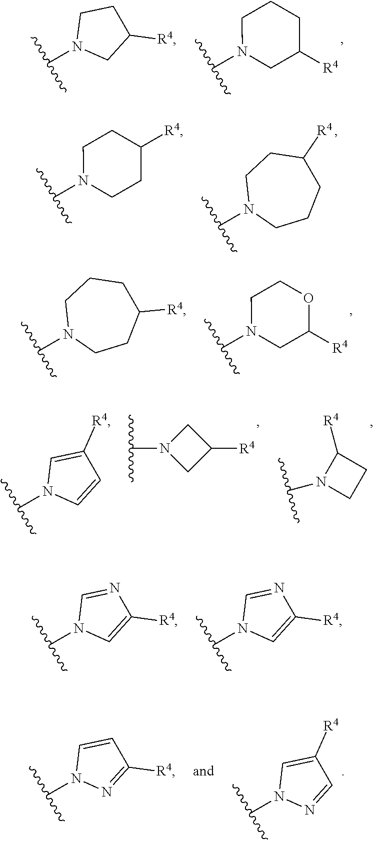

In one embodiment of Formula (I), (IA) (IB), (IV), or (IVA), including any of the foregoing, Ring A¹ is selected from

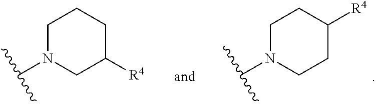

In one embodiment of Formula (I), (IA) (IB), (IV), or (IVA), including any of the foregoing, Ring A¹ is

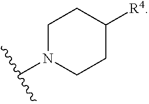

In one embodiment of Formula (I), (IA) (IB), (IV), or (IVA), including any of the foregoing, R⁴ is an 3- to 12-membered bridged or fused bicyclic heterocycle comprising 1, 2, 3, or 4 heteroatoms selected from N, S, and O wherein at least one heteroatom is NR⁵. In one embodiment of Formula (I), (IA) (IB), (IV), or (IVA), including any of the foregoing, R⁴ is an 8- to 12-membered bridged or fused bicyclic heterocycle comprising 1, 2, 3, or 4 heteroatoms selected from N, S, and O wherein at least one heteroatom is NR⁵. In one embodiment of Formula (I), (IA) (IB), (IV), or (IVA), including any of the foregoing, R⁴ is a 5-5 fused ring system, a 5-6 fused ring system, a 6-6 fused ring system, or a 5-7 fused ring system comprising 1, 2, 3, or 4 heteroatoms selected from N, S, and O wherein at least one heteroatom is NR⁵. In one embodiment of Formula (I), (IA) (IB), (IV), or (IVA), including any of the foregoing, R⁴ is a 5-6 fused ring system comprising 1, 2, 3, or 4 heteroatoms selected from N, S, and O wherein at least one heteroatom is NR⁵.

In one embodiment of Formula (I), (IA) (IB), (IV), or (IVA), including any of the foregoing, R⁴ is

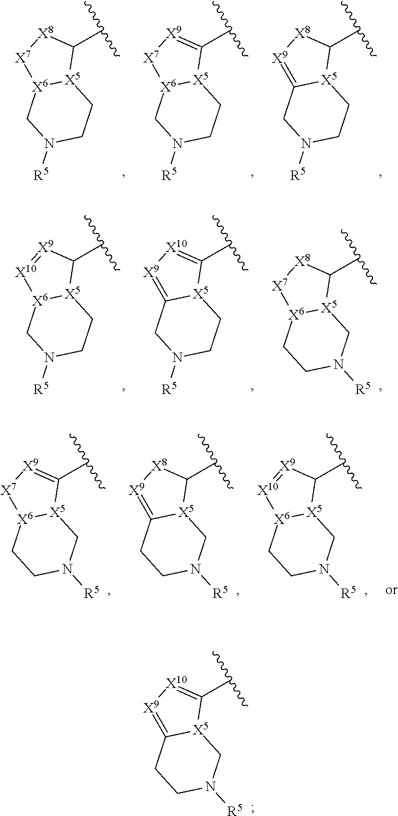

wherein X⁵, X⁶, X⁹, X¹⁰ are independently N or CR¹¹;

X⁷ and X⁸ are independently NH, O, or CHR¹¹;

R¹¹ is hydrogen or C₁₋₆ alkyl;

R⁵ is as defined herein; and

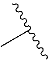

is the point of attachment to the rest of the compound;

wherein if four of any one of X⁵—X¹⁰ are present, at least one is CR¹¹ or CHR¹¹.

In one embodiment of Formula (I), (IA) (IB), (IV), or (IVA), including any of the foregoing, R⁴ is

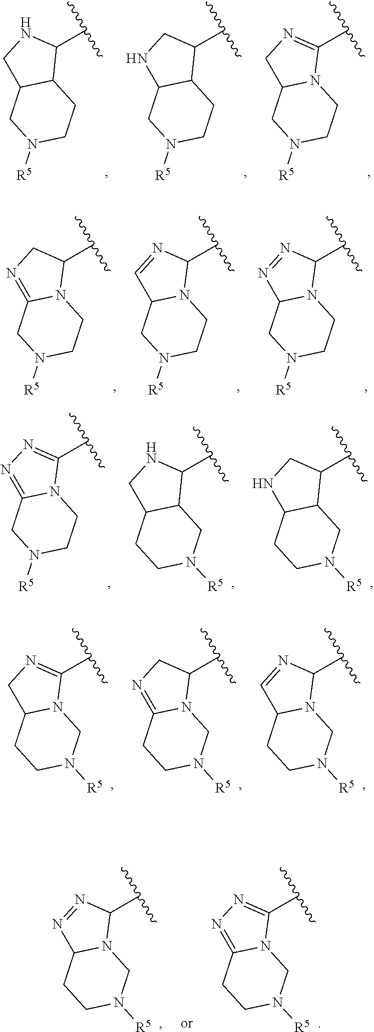

In one embodiment of Formula (I), (IA) (IB), (IV), or (IVA), including any of the foregoing, R⁴ is

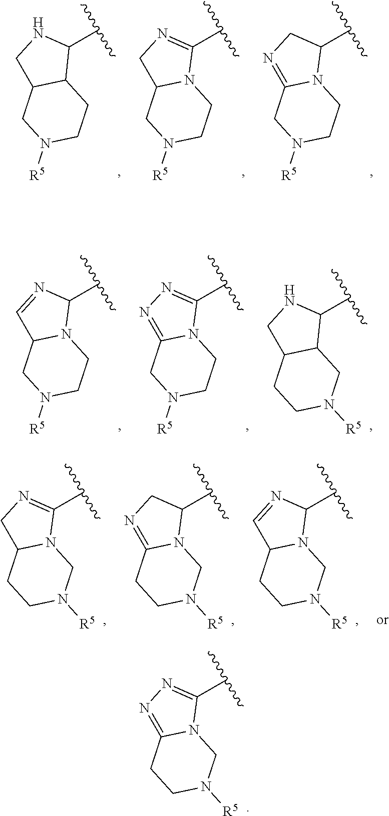

In one embodiment of Formula (I), (IA) (IB), (IV), or (IVA), including any of the foregoing, R⁴ is

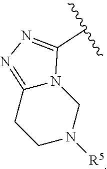

In one embodiment of Formula (I), (IA) (IB), (IV), or (IVA), including any of the foregoing, Ring A¹ is

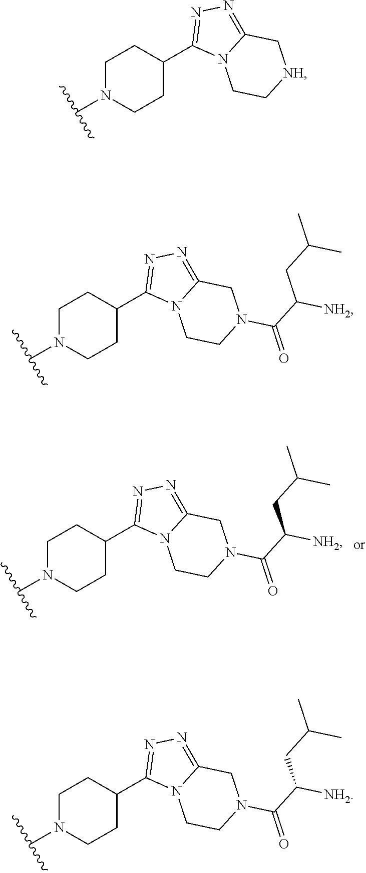 [SMILES: *N1CCC(c2nnc3n2CCNC3)CC1 ; *N1CCC(c2nnc3n2CCN(C(=O)C(N)CC(C)C)C3)CC1 ; *N1CCC(c2nnc3n2CCN(C(=O)[C@H](N)CC(C)C)C3)CC1 ; *N1CCC(c2nnc3n2CCN(C(=O)[C@@H](N)CC(C)C)C3)CC1]

In one embodiment of Formula (III) or (IIIA), including any of the foregoing, Ring A³ is an optionally substituted 3- to 12-membered heterocycle optionally substituted with one or more R⁵³. In one embodiment of Formula (III) or (IIIA), including any of the foregoing, Ring A³ is an optionally substituted 8- to 12-membered heterocycle optionally substituted with one or more R⁵³.

In one embodiment of Formula (III) or (IIIA), including any of the foregoing, Ring A³ is a N-linked monocyclic 3- to 12-membered heterocycle comprising the N bound to L² and NR⁵ and wherein the heterocycle is optionally substituted with one or more R⁵³. In one embodiment of Formula (III) or (IIIA), including any of the foregoing, Ring A³ is a N-linked monocyclic 3- to 12-membered heterocycle comprising the N bound to L² and NR⁵.

In one embodiment of Formula (III) or (IIIA), including any of the foregoing, Ring A³ is

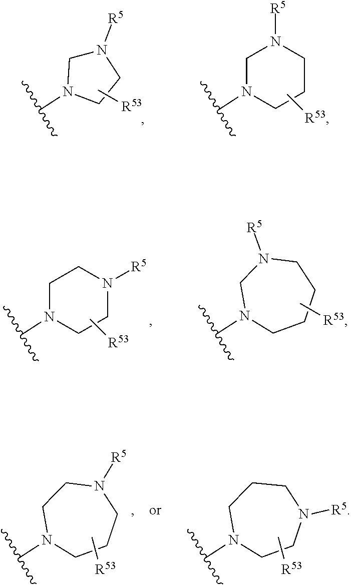

In one embodiment of Formula (III) or (IIIA), including any of the foregoing, Ring A³ is

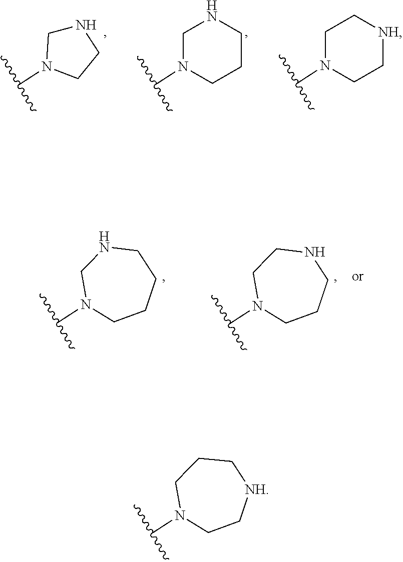 [SMILES: *N1CCNC1 ; *N1CCCNC1 ; *N1CCNCC1 ; *N1CCCCNC1 ; *N1CCCNCC1 ; *N1CCCNCC1]

In one embodiment of Formula (III) or (IIIA), including any of the foregoing, Ring A³ is

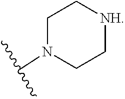 [SMILES: *N1CCNCC1]

In one embodiment of Formula (II), (IIA), (IIB), or (IIC), including any of the foregoing, L³ is C₁₋₃ alkyl. In one embodiment of Formula (II), (IIA), (IIB), or (IIC), including any of the foregoing, L³ is C₃₋₆ alkyl. In one embodiment of Formula (II), (IIA), (IIB), or (IIC), including any of the foregoing, L³ is C₃ alkyl.

In one embodiment of Formula (II), including any of the foregoing, L³ is C₁₋₆ alkyl substituted with one or more R⁵⁰. In one embodiment of Formula (II), (IIA), (IIB), or (IIC), including any of the foregoing, L³ is C₁₋₆ alkyl substituted with R^{8a} and R^{8b}.

In one embodiment of Formula (II), (IIA), (IIB), or (IIC), including any of the foregoing, L³ is C₁₋₆ alkyl substituted with R^{8a} and R^{8b} wherein R^{8a} and R^{8b} are joined together with the atoms to which they are attached to form a 3- to 12-membered heterocycle optionally substituted with one or more R⁵². In one embodiment of Formula (II), (IIA), (IIB), or (IIC), including any of the foregoing, L³ is C₁₋₆ alkyl substituted with R^{8a} and R^{8b} wherein R^{8a} and R^{8b} are joined together with the atoms to which they are attached to form a 4- to 6-membered heterocycle optionally substituted with one or more R⁵².

In one embodiment of Formula (II), (IIA), (IIB), or (IIC), including any of the foregoing, R^{8a} and R^{8b} are joined together with the atoms to which they are attached to form a 3- to 12-membered heterocycle containing 1, 2, 3, or 4 heteroatoms selected from N, S, and O wherein at least one heteroatom is NR⁵ wherein the heterocycle is optionally substituted with one or more R⁵². In one embodiment of Formula (II), (IIA), (IIB), or (IIC), including any of the foregoing, R^{8a} and R^{8b} are joined together with the atoms to which they are attached to form a 3- to 12-membered heterocycle containing 1, 2, 3, or 4 heteroatoms selected from N, S, and O wherein at least one heteroatom is NR⁵. In one embodiment of Formula (II), (IIA), (IIB), or (IIC), including any of the foregoing, R^{8a} and R^{8b} are joined together with the atoms to which they are attached to form a 4- to 6-membered heterocycle containing 1, 2, 3, or 4 heteroatoms selected from N, S, and O wherein at least one heteroatom is NR⁵.

In one embodiment of Formula (II), (IIA), (IIB), or (IIC), including any of the foregoing, R^{8a} and R^{8b} are joined together with the atoms to which they are attached to form

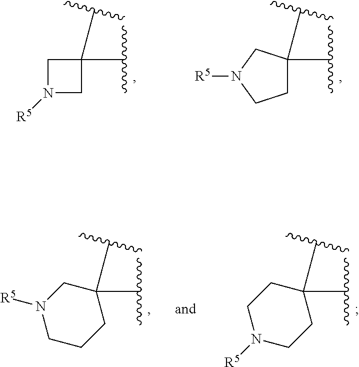

wherein each

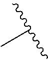

is a point of attachment to the rest of the compound and R⁵ is as defined herein. In one embodiment of Formula (II), (IIA), (IIB), or (IIC), including any of the foregoing, R^{8a} and R^{8b} are joined together with the atoms to which they are attached to form

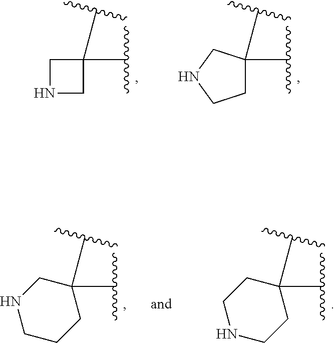 [SMILES: *C1(*)CNC1 ; *C1(*)CCNC1 ; *C1(*)CCCNC1 ; *C1(*)CCNCC1]

In one embodiment of Formula (II), (IIA), (IIB), or (IIC), including any of the foregoing, R^{8a} and R^{8b} are joined together with the atoms to which they are attached to form

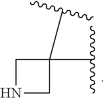 [SMILES: *C1(*)CNC1]

In one embodiment of Formula (I)—(IV), including any of the foregoing, R⁵ is hydrogen.

In one embodiment of Formula (I)—(IV), including any of the foregoing, R⁵ is —C(O)—C₁₋₆alkyl-NHR⁶⁰. In one embodiment of Formula (I)—(IV), including any of the foregoing, R⁵ is —C(O)—C₁₋₆alkyl-NH₂.

In one embodiment of Formula (I)—(IV), including any of the foregoing, R⁵ is —C(O)-heteroC₁₋₆alkyl-NHR⁶⁰. In one embodiment of Formula (I)—(IV), including any of the foregoing, R⁵ is —C(O)-heteroC₁₋₆alkyl-NH₂.

In one embodiment of Formula (I)—(IV), including any of the foregoing, R⁵ is —C(O)(CH₂CH₂O)_{b}NH₂. In one embodiment of Formula (I)—(IV), including any of the foregoing, R⁵ is —C(O)(CH₂CH₂O)_{b}NH₂ and b is an integer between 1 and 10, inclusive. In one embodiment Formula (I)—(IV), including any of the foregoing, b is an integer between 1 and 5, inclusive. In one embodiment of Formula (I)—(IV), including any of the foregoing, b is 4.

In one embodiment of Formula (I)—(IV), including any of the foregoing, R⁵ is —C(O)—C₁₋₆alkyl-O—NH₂. In one embodiment of Formula (I)—(IV), including any of the foregoing, R⁵ is —C(O)—(CH₂)₅—O—NH₂.

In one embodiment of Formula (I)—(IV), including any of the foregoing, R⁵ is R⁶ and R⁶ is an amino acid residue. In one embodiment of Formula (I)—(IV), including any of the foregoing, R⁶ is —(C(O)CHR^{6a}N^{7a})ₐ wherein R^{6a} is an amino acid sidechain residue; R^{7a} is hydrogen or C₁₋₆ alkyl; and a is an integer between 1 and 10, inclusive. In one embodiment of Formula (I)—(IV), including any of the foregoing, R⁶ is —C(O)CHR^{6a}NHR^{7a}. In one embodiment of Formula (I)—(IV), including any of the foregoing, R^{6a} is independently selected from a sidechain residue of valine and glycine and R^{7a} is hydrogen or methyl. The sidechain residue can have D- or L-stereochemistry.

Additional non-limiting examples of Formula (IA) include:

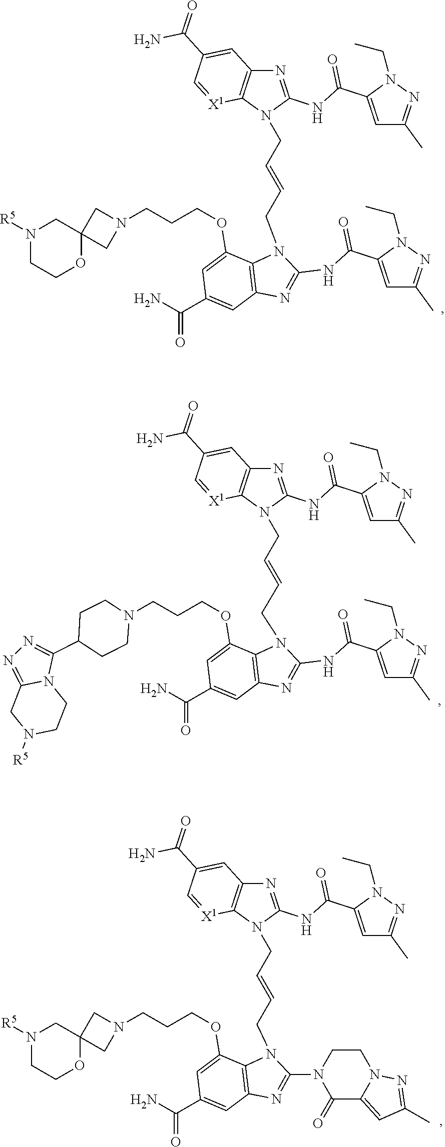

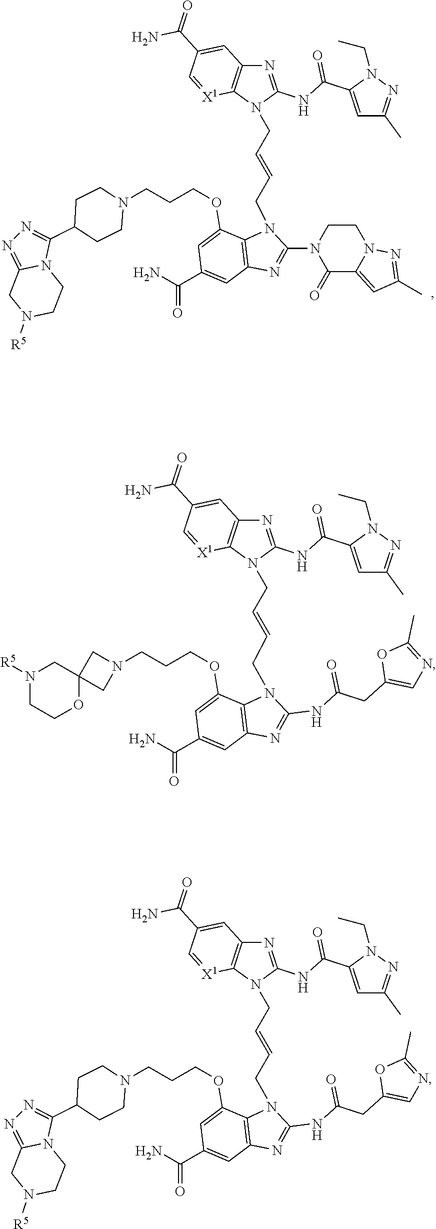

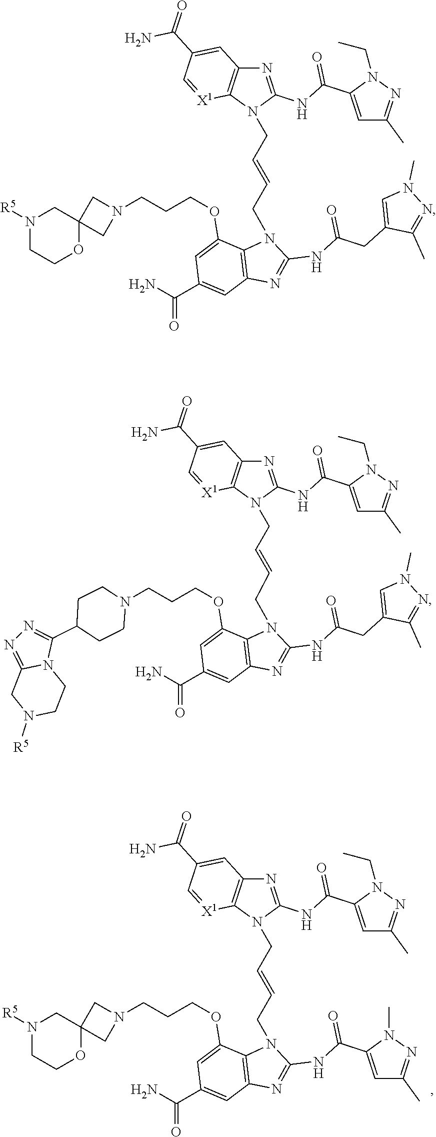

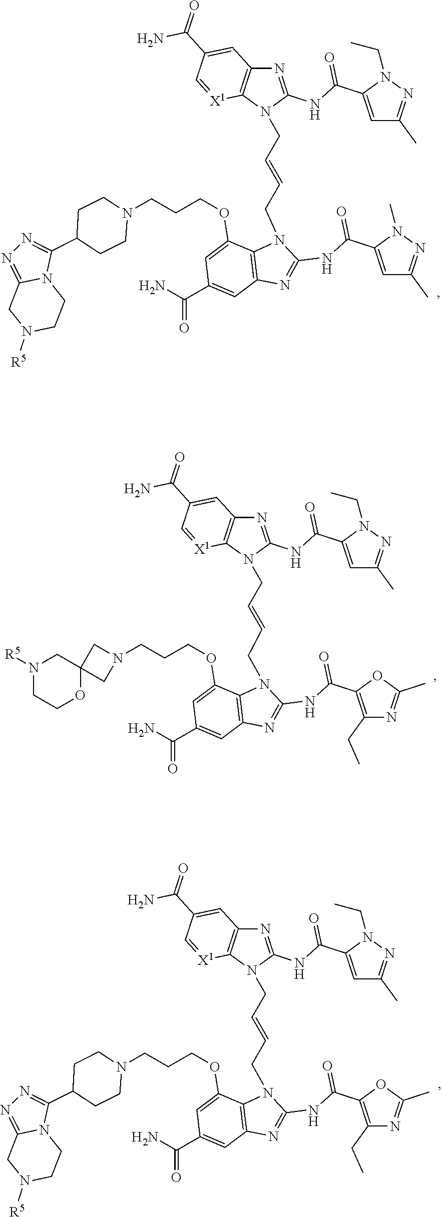

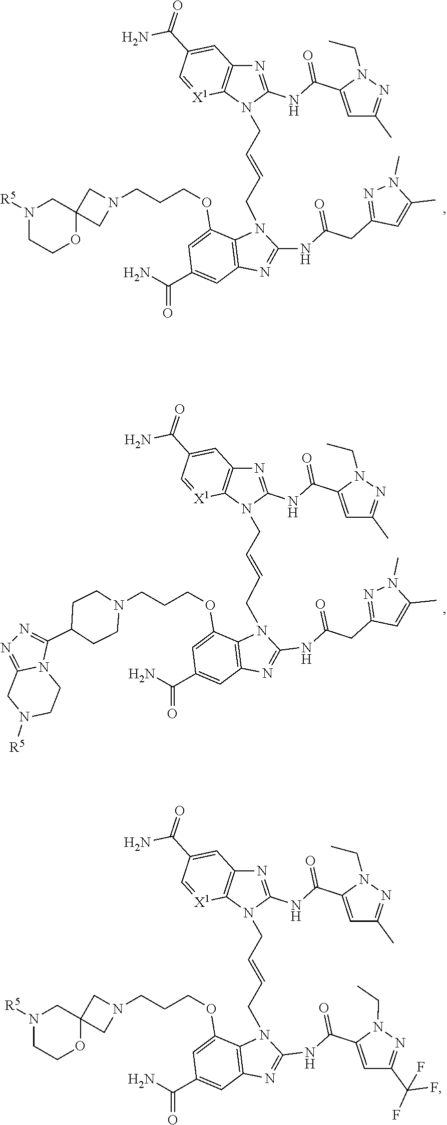

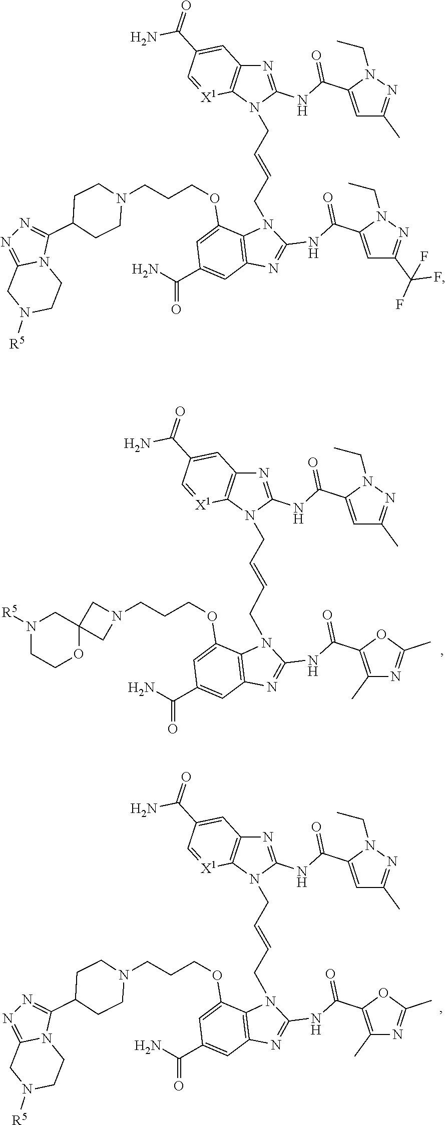

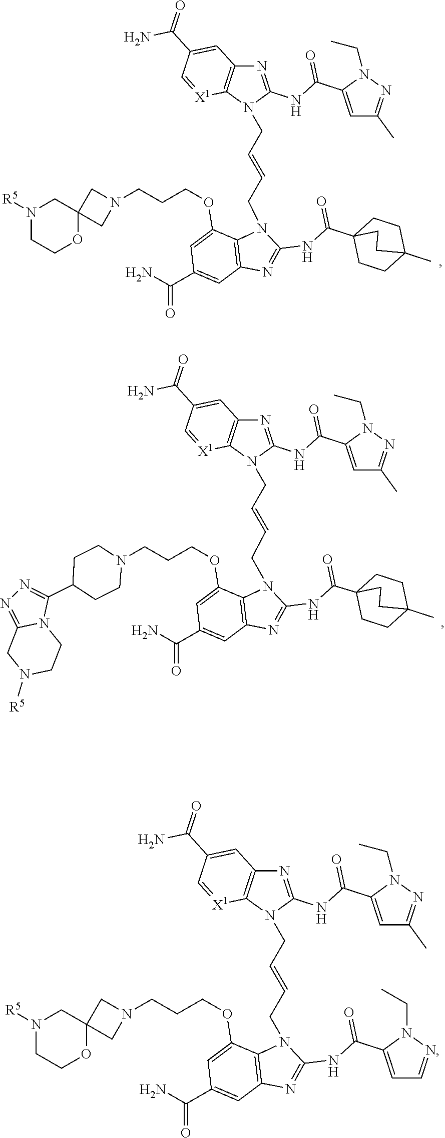

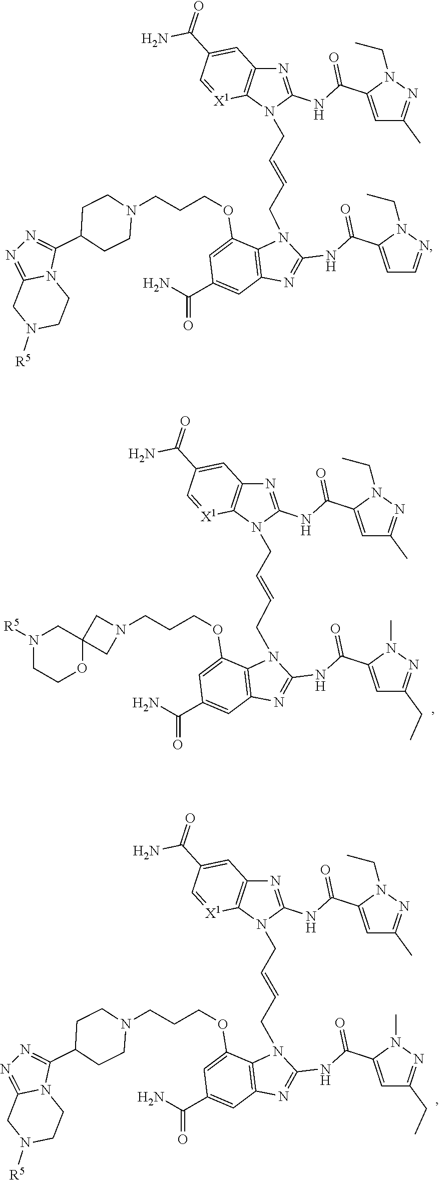

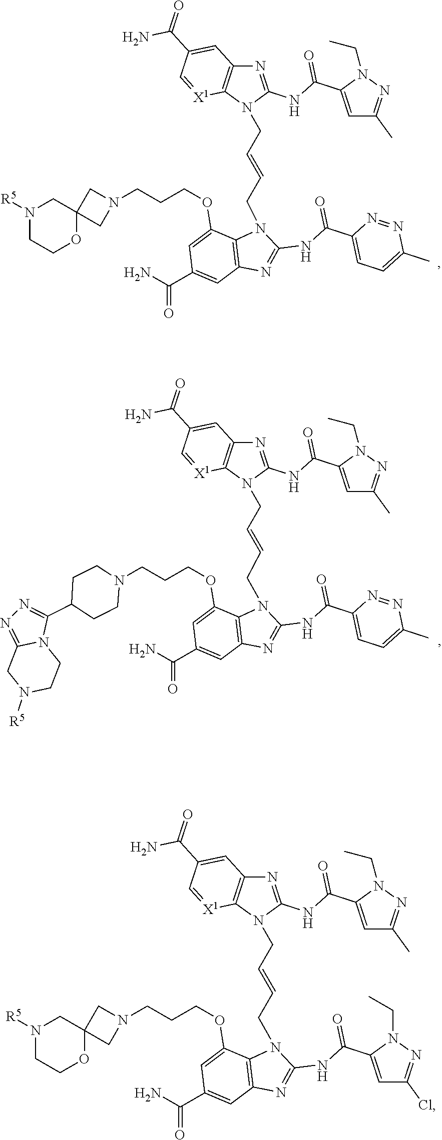

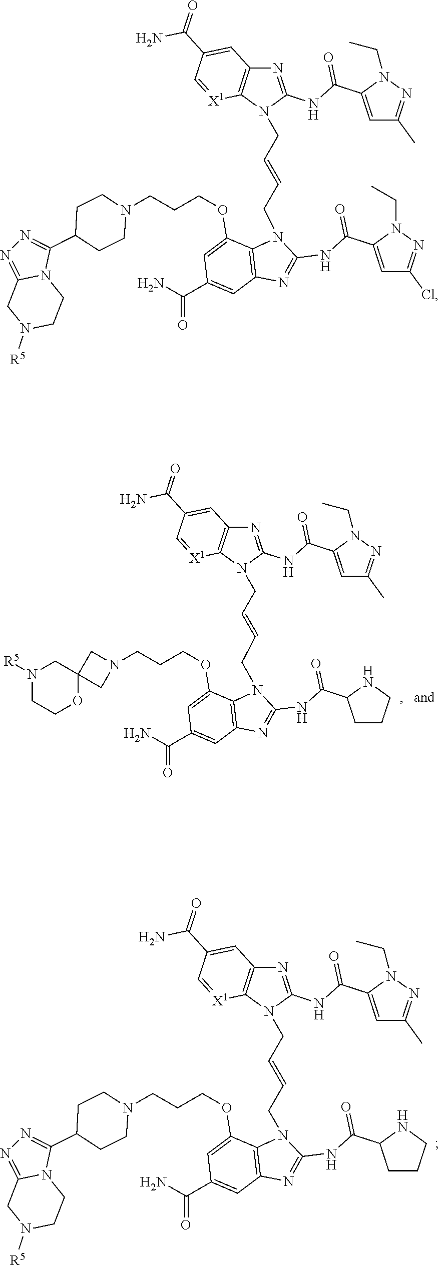

or a pharmaceutically acceptable salt or tautomer thereof.

Additional non-limiting examples of Formula (IB) include:

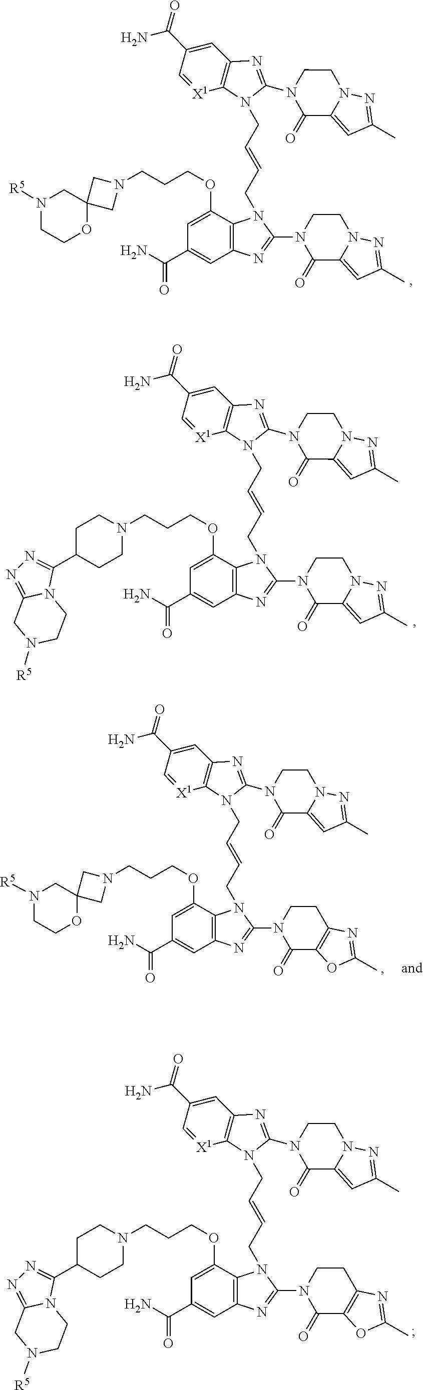

or a pharmaceutically acceptable salt or tautomer thereof.

Additional non-limiting examples of Formula (IIIA) include:

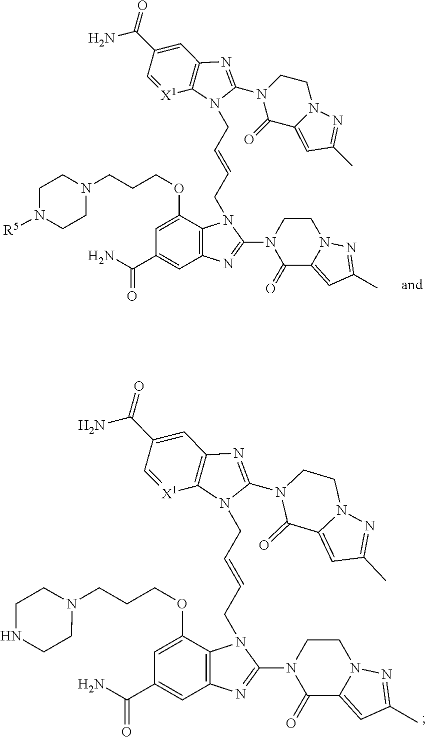

or a pharmaceutically acceptable salt or tautomer thereof.

A non-limiting example of Formula (IVA) includes:

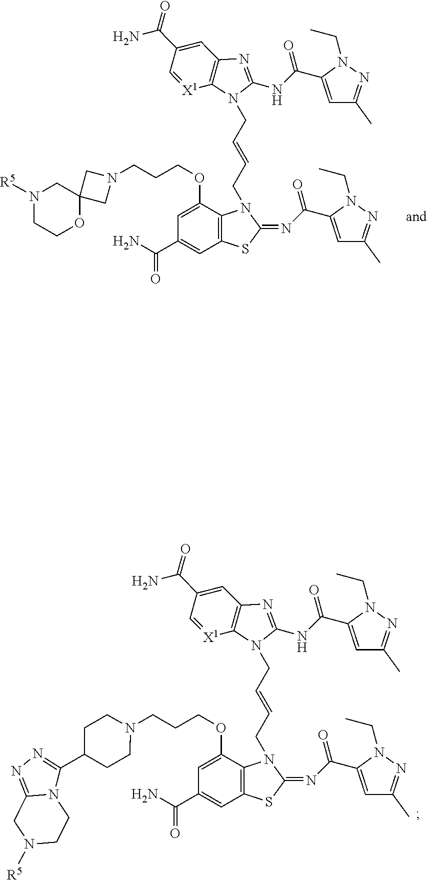

or a pharmaceutically acceptable salt or tautomer thereof.

In one embodiment of Formula (I), L¹ is —CH═CH—; R^{1a} and R^{1b} are both hydrogen; R^{2a} is

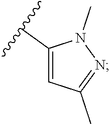 [SMILES: *c1cc(C)nn1C]

### R^{2b} is

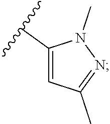 [SMILES: *c1cc(C)nn1C]

### and Ring A¹ is

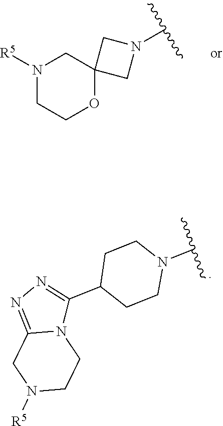

In one embodiment of Formula (I), L¹ is —CH═CH—; R^{1a} and R^{1b} are both hydrogen; R^{2a} is

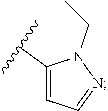 [SMILES: *c1ccnn1CC]

### R^{2b} is

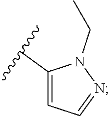 [SMILES: *c1ccnn1CC]

### and Ring A¹ is

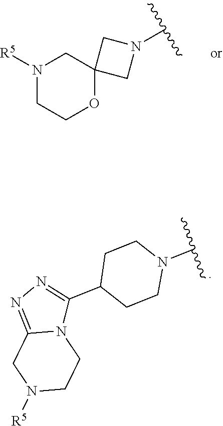

In one embodiment of Formula (I), L¹ is —CH═CH—; R^{1a} and R^{1b} are both hydrogen; R^{2a} is

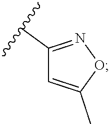 [SMILES: *c1cc(C)on1]

### R^{2b} is

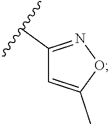 [SMILES: *c1cc(C)on1]

### and Ring A¹ is

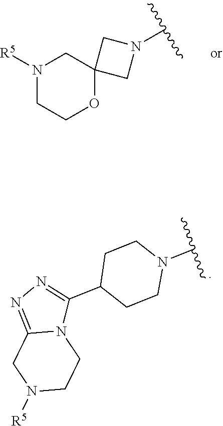

In one embodiment of Formula (I), L¹ is —CH═CH—; R^{1a} and R^{1b} are both hydrogen; R^{2a} is

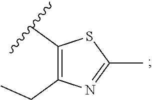 [SMILES: *c1sc(C)nc1CC]

### R^{2b} is

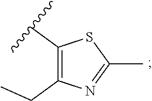 [SMILES: *c1sc(C)nc1CC]

### and Ring A¹ is

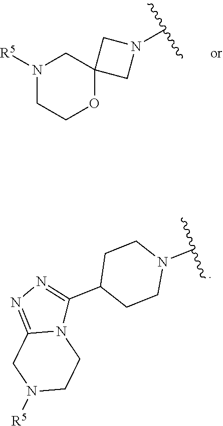

In one embodiment of Formula (I), L¹ is —CH═CH—; R^{1a} and R^{1b} are both hydrogen; R^{2a} is

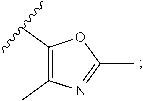 [SMILES: *c1oc(C)nc1C]

### R^{2b} is

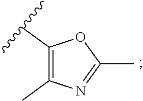 [SMILES: *c1oc(C)nc1C]

### and Ring A¹ is

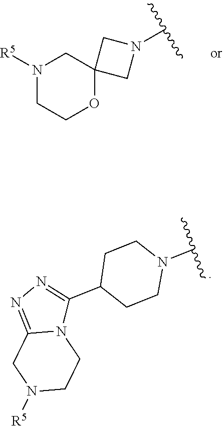

In one embodiment of Formula (I), L¹ is —CH═CH—; R^{1a} and R^{1b} are both hydrogen; R^{2a} is

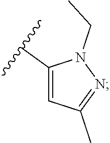 [SMILES: *c1cc(C)nn1CC]

### R^{2b} is

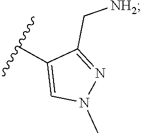 [SMILES: *c1cn(C)nc1CN]

### and Ring A¹ is

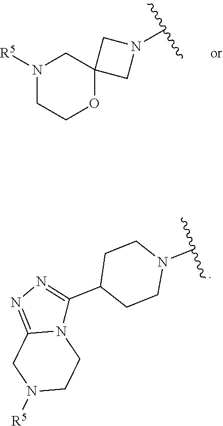

In one embodiment of Formula (I), L¹ is —CH═CH—; R^{1a} and R^{1b} are both hydrogen; R^{2a} is

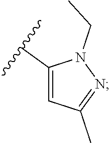 [SMILES: *c1cc(C)nn1CC]

### R^{2b} is

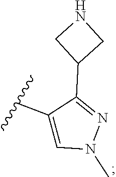 [SMILES: *c1cn(C)nc1C1CNC1]

### and Ring A¹ is

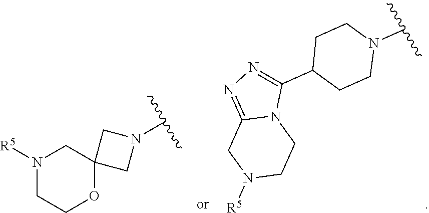

In one embodiment of Formula (I), L¹ is —CH₂CH₂—; R^{1a} and R^{1b} are both hydrogen; R^{2a} is

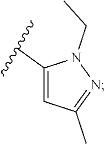 [SMILES: *c1cc(C)nn1CC]

### R^{2b} is

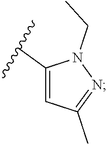 [SMILES: *c1cc(C)nn1CC]

### and Ring A¹ is

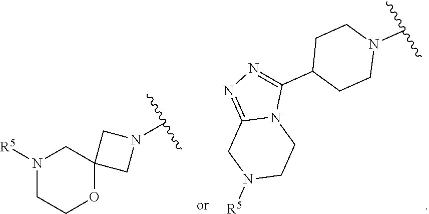

In one embodiment of Formula (I), L¹ is —CH═CH—; R²⁰ is hydrogen; R^{1a} and R^{1b} are both hydrogen; R^{2a} and R^{2b} are both

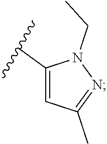 [SMILES: *c1cc(C)nn1CC]

### and Ring A¹ is

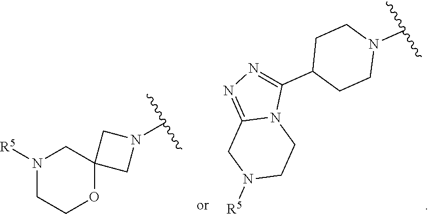

In one embodiment of Formula (II), including any of the foregoing, L¹ is —CH═CH—; R^{1a} and R^{1b} are both hydrogen; R^{2a} and R^{2b} are both

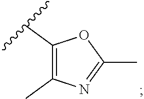

and R^{8a} and R^{8b} are joined together with the atoms to which they are attached to form

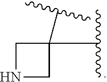 [SMILES: *C1(*)CNC1]

In one embodiment, the compound of Formula (I), (II), (III) is a compound or pharmaceutically acceptable salt or tautomer thereof of Table A or Table A-1 or Table A-2:

TABLE A

| Compound | |
|---|---|
| No. | Structure |
| 1 | 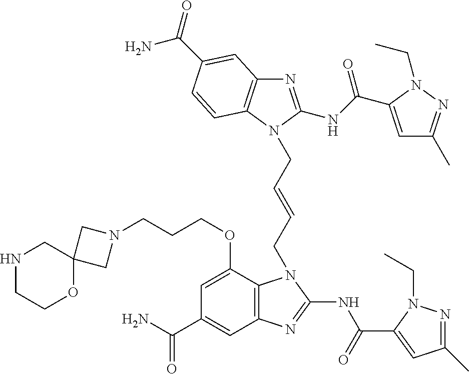 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(CNCCO4)C3)c21] |
| 2 | 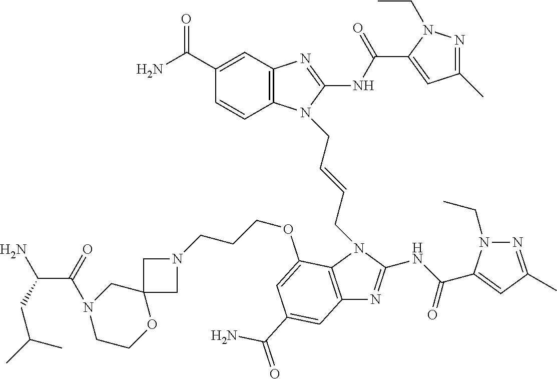 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@@H](N)CC(C)C)CCO4)c21] |
| 3 | 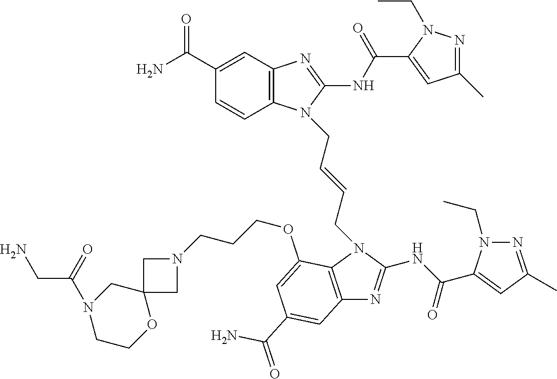 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)CN)CCO4)c21] |
| 4 | 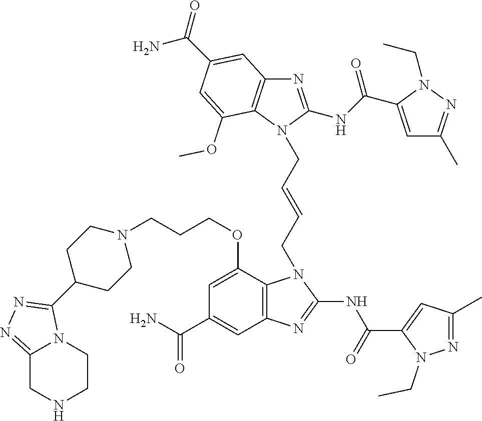 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCNC5)CC3)c21] |
| 5 | 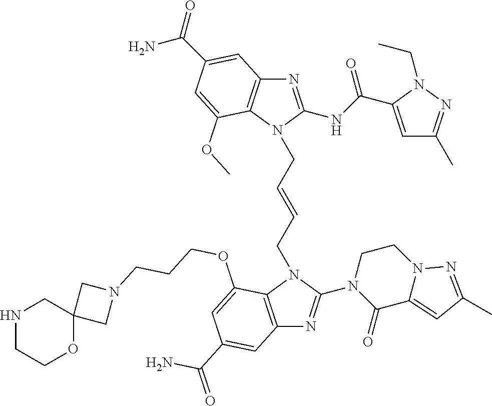 [SMILES: CCn1nc(C)cc1C(=O)Nc1nc2cc(C(N)=O)cc(OC)c2n1C/C=C/Cn1c(N2CCn3nc(C)cc3C2=O)nc2cc(C(N)=O)cc(OCCCN3CC4(CNCCO4)C3)c21] |
| 6 | 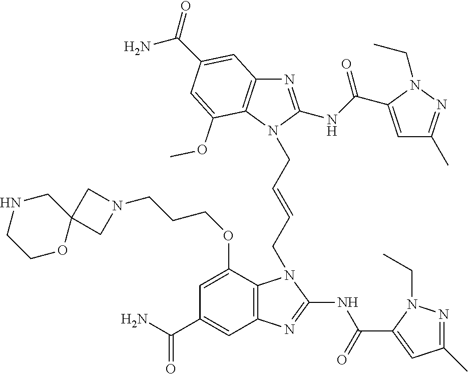 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(CNCCO4)C3)c21] |
| 7 | 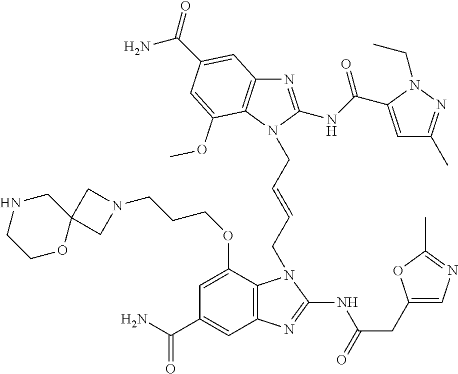 [SMILES: CCn1nc(C)cc1C(=O)Nc1nc2cc(C(N)=O)cc(OC)c2n1C/C=C/Cn1c(NC(=O)Cc2cnc(C)o2)nc2cc(C(N)=O)cc(OCCCN3CC4(CNCCO4)C3)c21] |
| 8 | 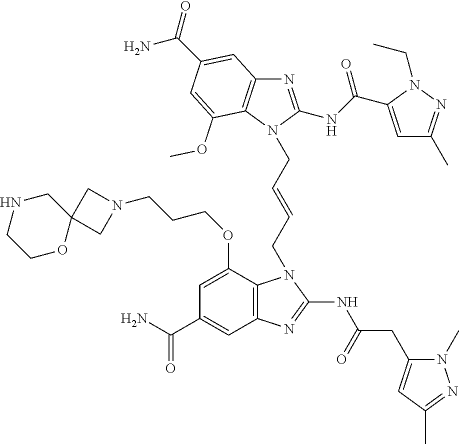 [SMILES: CCn1nc(C)cc1C(=O)Nc1nc2cc(C(N)=O)cc(OC)c2n1C/C=C/Cn1c(NC(=O)Cc2cc(C)nn2C)nc2cc(C(N)=O)cc(OCCCN3CC4(CNCCO4)C3)c21] |
| 9 | 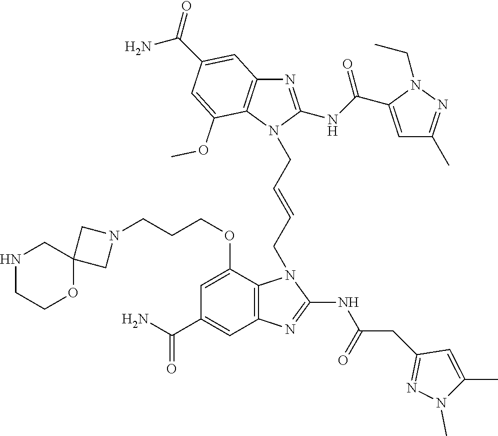 [SMILES: CCn1nc(C)cc1C(=O)Nc1nc2cc(C(N)=O)cc(OC)c2n1C/C=C/Cn1c(NC(=O)Cc2cc(C)n(C)n2)nc2cc(C(N)=O)cc(OCCCN3CC4(CNCCO4)C3)c21] |
| 10 | 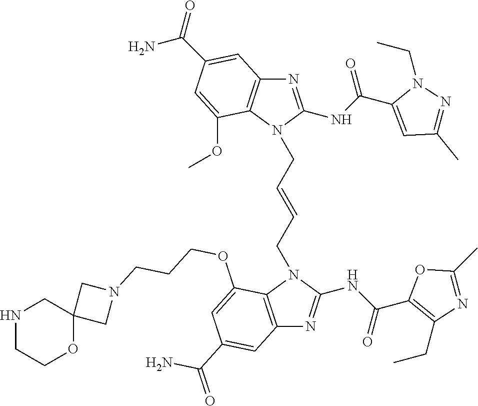 [SMILES: CCc1nc(C)oc1C(=O)Nc1nc2cc(C(N)=O)cc(OCCCN3CC4(CNCCO4)C3)c2n1C/C=C/Cn1c(NC(=O)c2cc(C)nn2CC)nc2cc(C(N)=O)cc(OC)c21] |
| 11 | 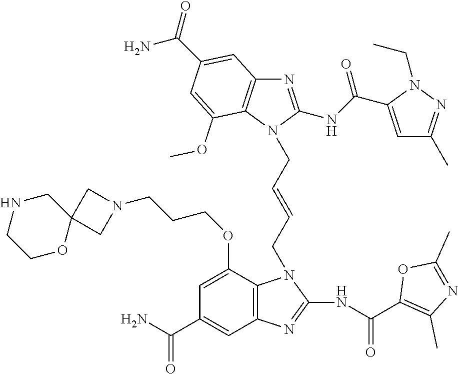 [SMILES: CCn1nc(C)cc1C(=O)Nc1nc2cc(C(N)=O)cc(OC)c2n1C/C=C/Cn1c(NC(=O)c2oc(C)nc2C)nc2cc(C(N)=O)cc(OCCCN3CC4(CNCCO4)C3)c21] |
| 12 | 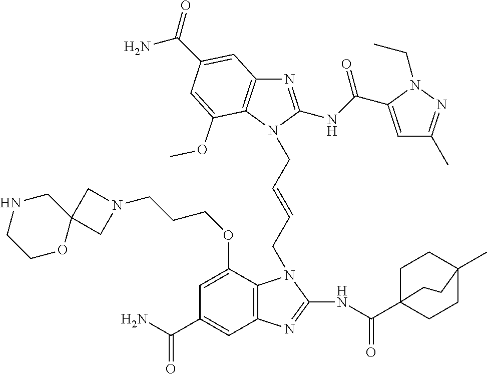 [SMILES: CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(CNCCO4)C3)c21] |
| 13 | 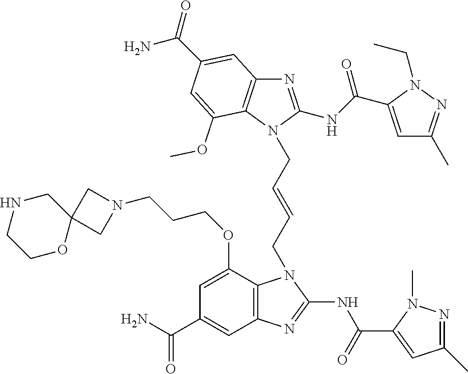 [SMILES: CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCN3CC4(CNCCO4)C3)c21] |
| 14 | 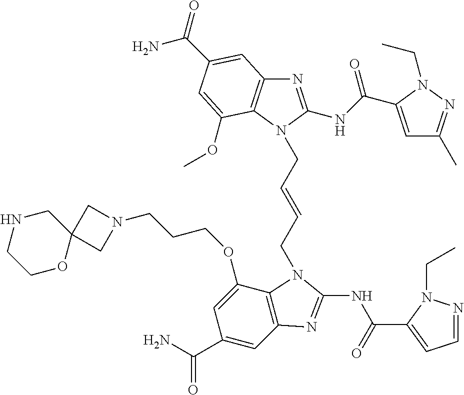 [SMILES: CCn1nc(C)cc1C(=O)Nc1nc2cc(C(N)=O)cc(OC)c2n1C/C=C/Cn1c(NC(=O)c2ccnn2CC)nc2cc(C(N)=O)cc(OCCCN3CC4(CNCCO4)C3)c21] |
| 15 | 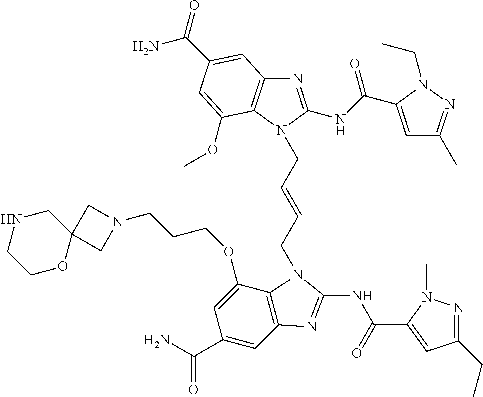 [SMILES: CCc1cc(C(=O)Nc2nc3cc(C(N)=O)cc(OCCCN4CC5(CNCCO5)C4)c3n2C/C=C/Cn2c(NC(=O)c3cc(C)nn3CC)nc3cc(C(N)=O)cc(OC)c32)n(C)n1] |
| 16 | 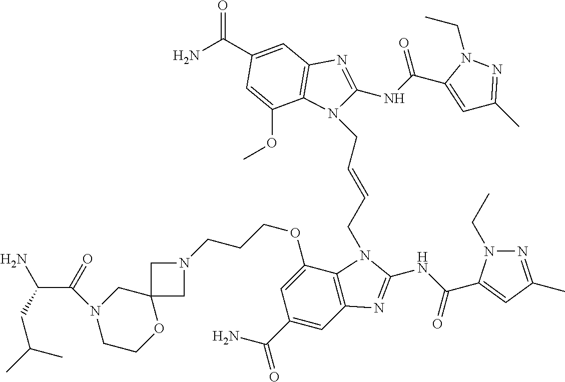 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(C3)CN(C(=O)[C@@H](N)CC(C)C)CCO4)c21] |
| 17 | 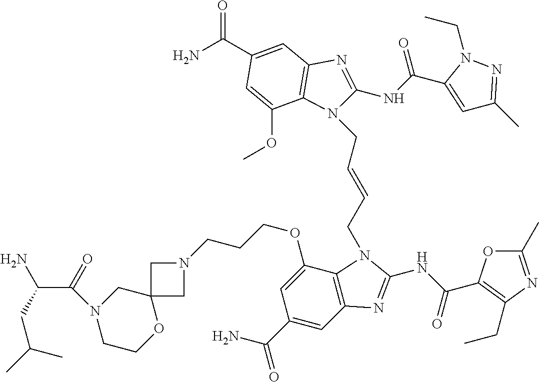 [SMILES: CCc1nc(C)oc1C(=O)Nc1nc2cc(C(N)=O)cc(OCCCN3CC4(C3)CN(C(=O)[C@@H](N)CC(C)C)CCO4)c2n1C/C=C/Cn1c(NC(=O)c2cc(C)nn2CC)nc2cc(C(N)=O)cc(OC)c21] |
| 18 | 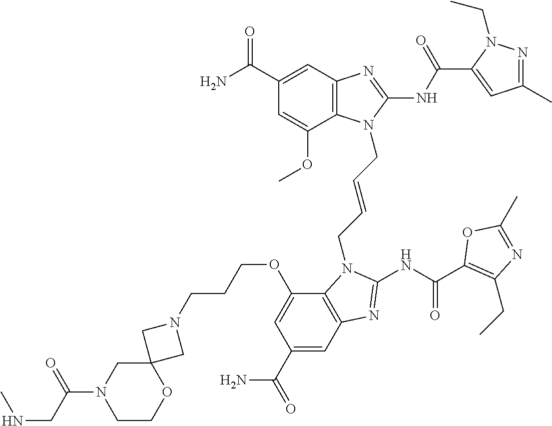 |
| 19 | 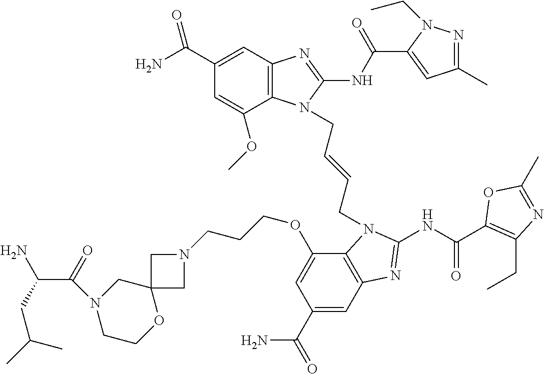 [SMILES: CCc1nc(C)oc1C(=O)Nc1nc2cc(C(N)=O)cc(OCCCN3CC4(C3)CN(C(=O)[C@@H](N)CC(C)C)CCO4)c2n1C/C=C/Cn1c(NC(=O)c2cc(C)nn2CC)nc2cc(C(N)=O)cc(OC)c21] |
| 20 | 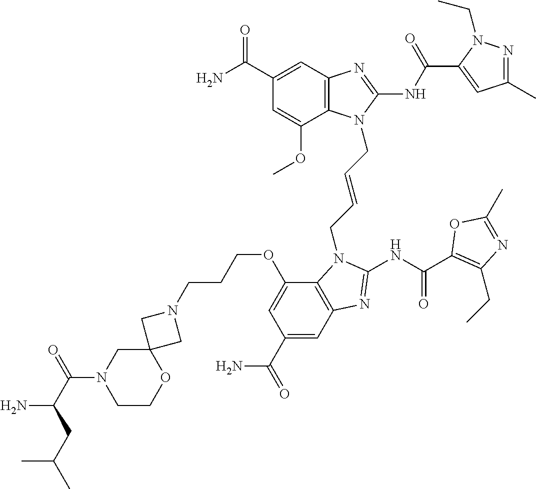 [SMILES: CCc1nc(C)oc1C(=O)Nc1nc2cc(C(N)=O)cc(OCCCN3CC4(C3)CN(C(=O)[C@H](N)CC(C)C)CCO4)c2n1C/C=C/Cn1c(NC(=O)c2cc(C)nn2CC)nc2cc(C(N)=O)cc(OC)c21] |
| 21 | 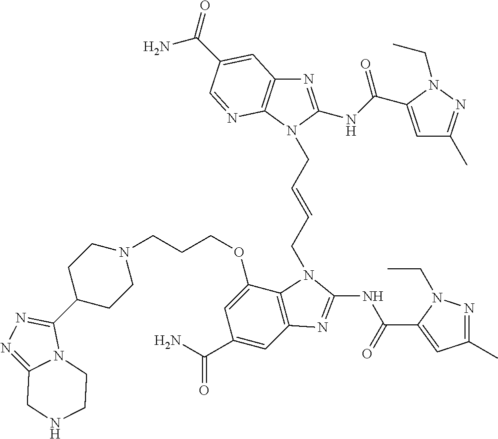 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCNC5)CC3)c21] |
| 22 | 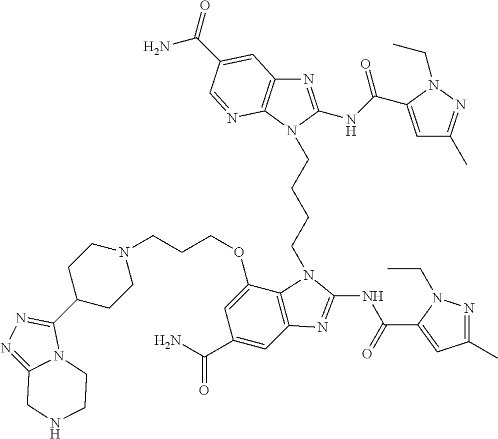 [SMILES: CCn1nc(C)cc1C(=O)Nc1nc2cc(C(N)=O)cnc2n1CCCCn1c(NC(=O)c2cc(C)nn2CC)nc2cc(C(N)=O)cc(OCCCN3CCC(c4nnc5n4CCNC5)CC3)c21] |
| 23 | 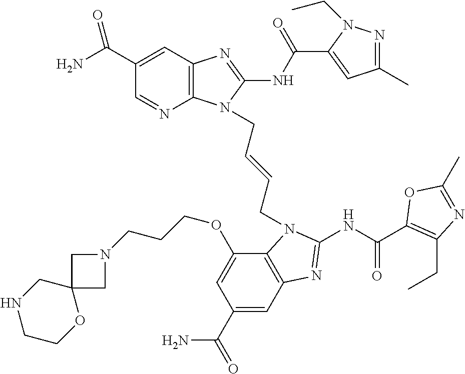 [SMILES: CCc1nc(C)oc1C(=O)Nc1nc2cc(C(N)=O)cc(OCCCN3CC4(CNCCO4)C3)c2n1C/C=C/Cn1c(NC(=O)c2cc(C)nn2CC)nc2cc(C(N)=O)cnc21] |
| 24 | 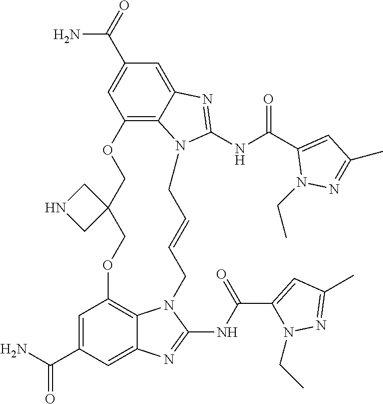 [SMILES: CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC1(CNC1)CO3] |
| 25 | 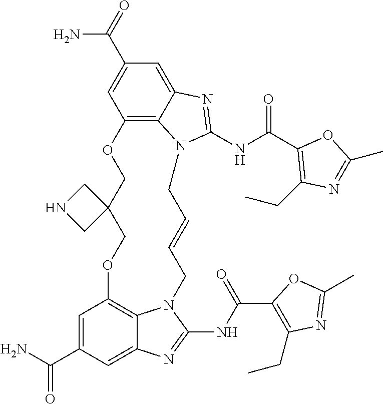 [SMILES: CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CNC1)CO3] |
| 26 | 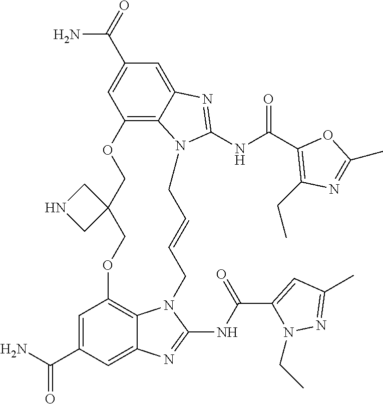 [SMILES: CCc1nc(C)oc1C(=O)Nc1nc2cc(C(N)=O)cc3c2n1C/C=C/Cn1c(NC(=O)c2cc(C)nn2CC)nc2cc(C(N)=O)cc(c21)OCC1(CNC1)CO3] |
| 27 | 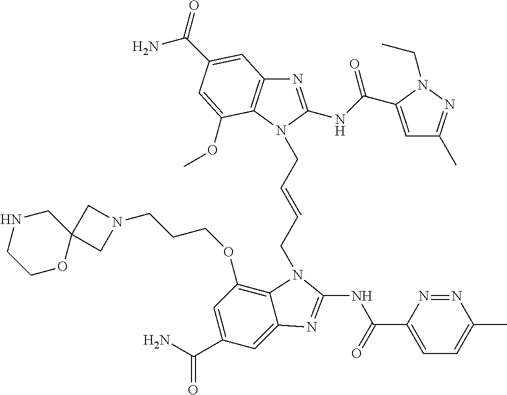 [SMILES: CCn1nc(C)cc1C(=O)Nc1nc2cc(C(N)=O)cc(OC)c2n1C/C=C/Cn1c(NC(=O)c2ccc(C)nn2)nc2cc(C(N)=O)cc(OCCCN3CC4(CNCCO4)C3)c21] |
| 28 | 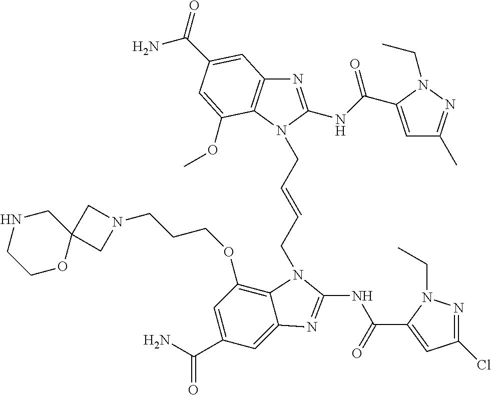 [SMILES: CCn1nc(C)cc1C(=O)Nc1nc2cc(C(N)=O)cc(OC)c2n1C/C=C/Cn1c(NC(=O)c2cc(Cl)nn2CC)nc2cc(C(N)=O)cc(OCCCN3CC4(CNCCO4)C3)c21] |
| 29 | 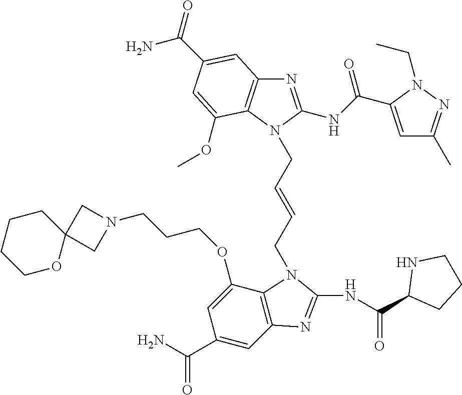 [SMILES: CCn1nc(C)cc1C(=O)Nc1nc2cc(C(N)=O)cc(OC)c2n1C/C=C/Cn1c(NC(=O)[C@@H]2CCCN2)nc2cc(C(N)=O)cc(OCCCN3CC4(CCCCO4)C3)c21] |
| 30 | 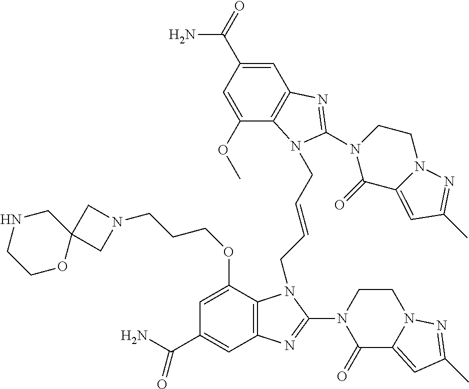 [SMILES: COc1cc(C(N)=O)cc2nc(N3CCn4nc(C)cc4C3=O)n(C/C=C/Cn3c(N4CCn5nc(C)cc5C4=O)nc4cc(C(N)=O)cc(OCCCN5CC6(CNCCO6)C5)c43)c12] |
| 31 | 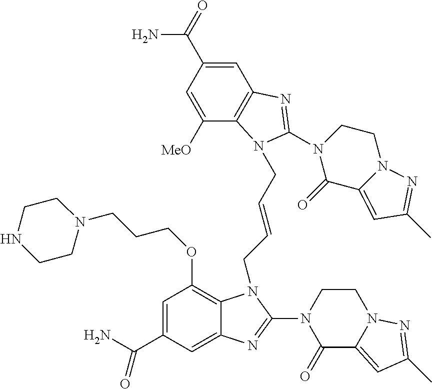 [SMILES: COc1cc(C(N)=O)cc2nc(N3CCn4nc(C)cc4C3=O)n(C/C=C/Cn3c(N4CCn5nc(C)cc5C4=O)nc4cc(C(N)=O)cc(OCCCN5CCNCC5)c43)c12] |
| 32 | 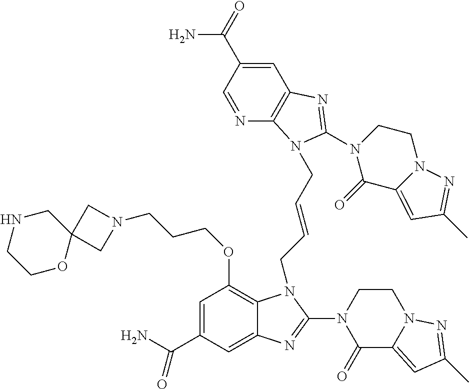 [SMILES: Cc1cc2n(n1)CCN(c1nc3cc(C(N)=O)cnc3n1C/C=C/Cn1c(N3CCn4nc(C)cc4C3=O)nc3cc(C(N)=O)cc(OCCCN4CC5(CNCCO5)C4)c31)C2=O] |
| 33 | 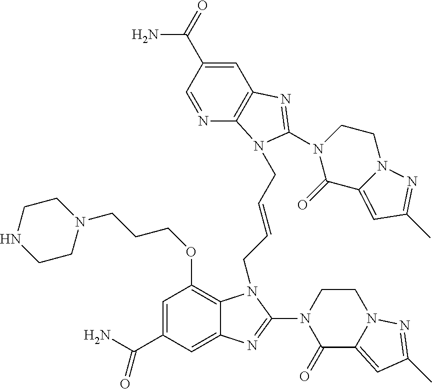 [SMILES: Cc1cc2n(n1)CCN(c1nc3cc(C(N)=O)cnc3n1C/C=C/Cn1c(N3CCn4nc(C)cc4C3=O)nc3cc(C(N)=O)cc(OCCCN4CCNCC4)c31)C2=O] |
| 34 | 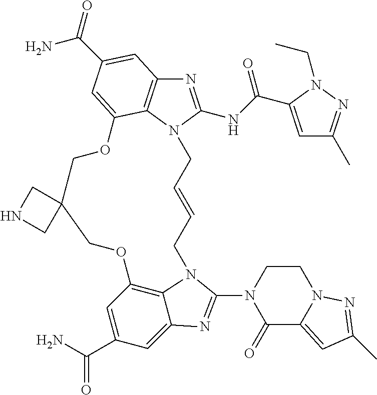 [SMILES: CCn1nc(C)cc1C(=O)Nc1nc2cc(C(N)=O)cc3c2n1C/C=C/Cn1c(N2CCn4nc(C)cc4C2=O)nc2cc(C(N)=O)cc(c21)OCC1(CNC1)CO3] |
| 35 | 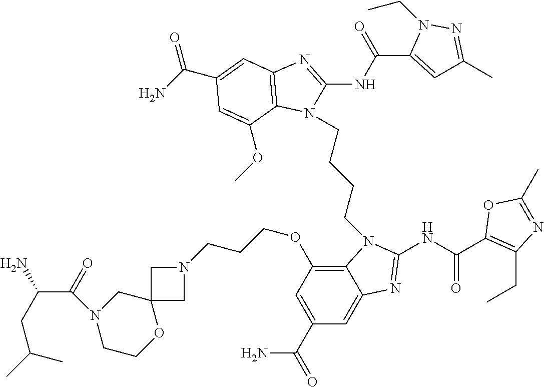 [SMILES: CCc1nc(C)oc1C(=O)Nc1nc2cc(C(N)=O)cc(OCCCN3CC4(C3)CN(C(=O)[C@@H](N)CC(C)C)CCO4)c2n1CCCCn1c(NC(=O)c2cc(C)nn2CC)nc2cc(C(N)=O)cc(OC)c21] |
| 36 | 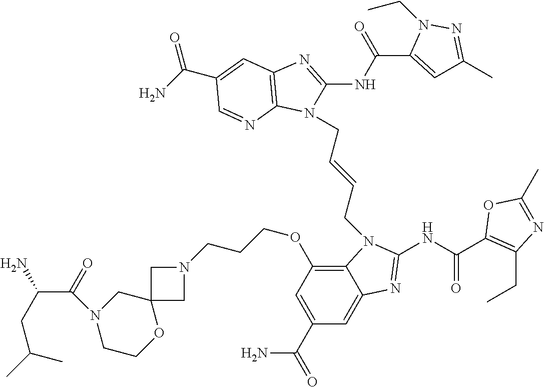 [SMILES: CCc1nc(C)oc1C(=O)Nc1nc2cc(C(N)=O)cc(OCCCN3CC4(C3)CN(C(=O)[C@@H](N)CC(C)C)CCO4)c2n1C/C=C/Cn1c(NC(=O)c2cc(C)nn2CC)nc2cc(C(N)=O)cnc21] |
| 37 | 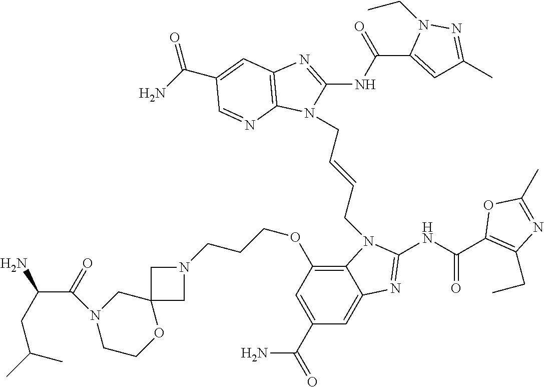 [SMILES: CCc1nc(C)oc1C(=O)Nc1nc2cc(C(N)=O)cc(OCCCN3CC4(C3)CN(C(=O)[C@H](N)CC(C)C)CCO4)c2n1C/C=C/Cn1c(NC(=O)c2cc(C)nn2CC)nc2cc(C(N)=O)cnc21] |
| 38 | 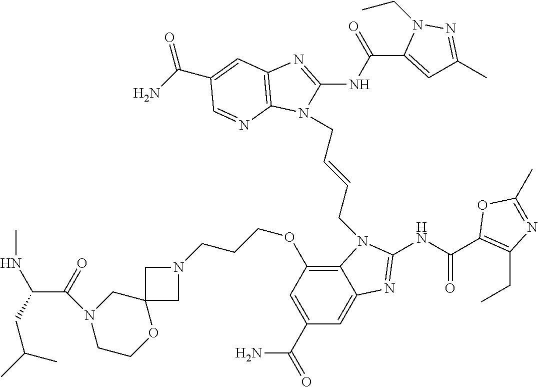 [SMILES: CCc1nc(C)oc1C(=O)Nc1nc2cc(C(N)=O)cc(OCCCN3CC4(C3)CN(C(=O)[C@H](CC(C)C)NC)CCO4)c2n1C/C=C/Cn1c(NC(=O)c2cc(C)nn2CC)nc2cc(C(N)=O)cnc21] |
| 39 | 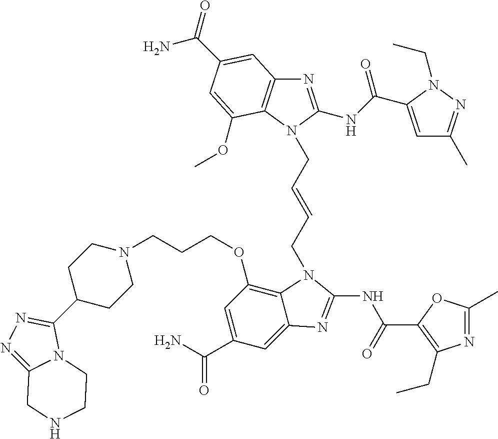 [SMILES: CCc1nc(C)oc1C(=O)Nc1nc2cc(C(N)=O)cc(OCCCN3CCC(c4nnc5n4CCNC5)CC3)c2n1C/C=C/Cn1c(NC(=O)c2cc(C)nn2CC)nc2cc(C(N)=O)cc(OC)c21] |
| 40 | 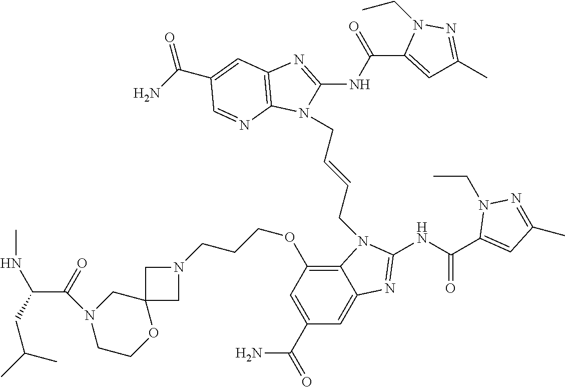 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CC4(C3)CN(C(=O)[C@H](CC(C)C)NC)CCO4)c21] |
| 41 | 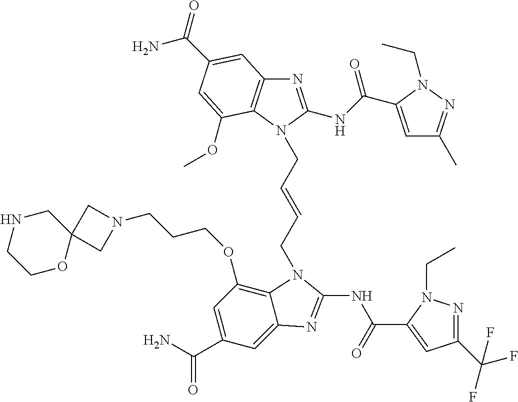 [SMILES: CCn1nc(C)cc1C(=O)Nc1nc2cc(C(N)=O)cc(OC)c2n1C/C=C/Cn1c(NC(=O)c2cc(C(F)(F)F)nn2CC)nc2cc(C(N)=O)cc(OCCCN3CC4(CNCCO4)C3)c21] |
| 42 | 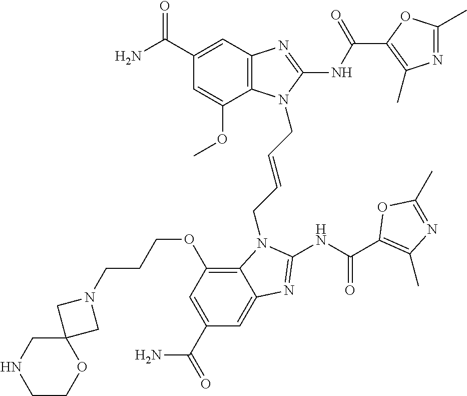 [SMILES: COc1cc(C(N)=O)cc2nc(NC(=O)c3oc(C)nc3C)n(C/C=C/Cn3c(NC(=O)c4oc(C)nc4C)nc4cc(C(N)=O)cc(OCCCN5CC6(CNCCO6)C5)c43)c12] |
| 43 | 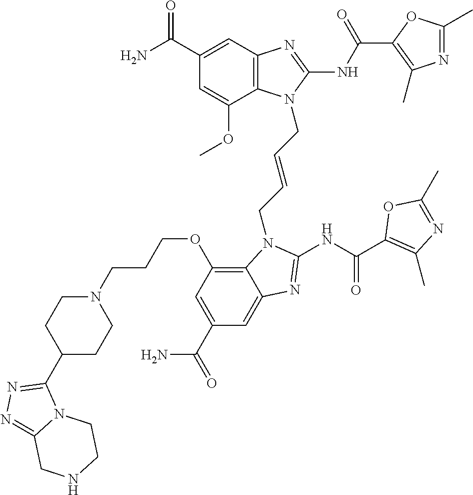 [SMILES: COc1cc(C(N)=O)cc2nc(NC(=O)c3oc(C)nc3C)n(C/C=C/Cn3c(NC(=O)c4oc(C)nc4C)nc4cc(C(N)=O)cc(OCCCN5CCC(c6nnc7n6CCNC7)CC5)c43)c12] |
| 44 | 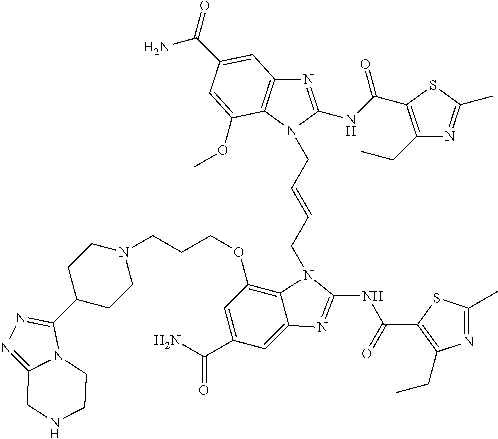 [SMILES: CCc1nc(C)sc1C(=O)Nc1nc2cc(C(N)=O)cc(OC)c2n1C/C=C/Cn1c(NC(=O)c2sc(C)nc2CC)nc2cc(C(N)=O)cc(OCCCN3CCC(c4nnc5n4CCNC5)CC3)c21] |
| 45 | 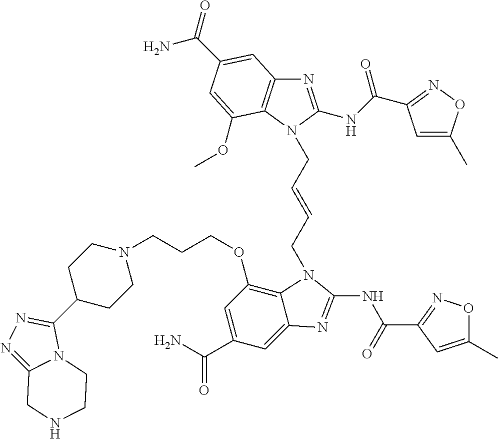 [SMILES: COc1cc(C(N)=O)cc2nc(NC(=O)c3cc(C)on3)n(C/C=C/Cn3c(NC(=O)c4cc(C)on4)nc4cc(C(N)=O)cc(OCCCN5CCC(c6nnc7n6CCNC7)CC5)c43)c12] |
| 46 | 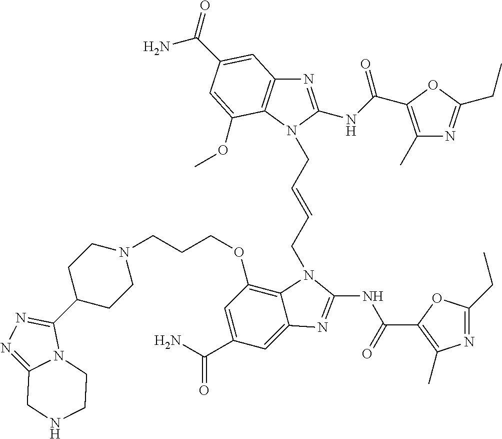 [SMILES: CCc1nc(C)c(C(=O)Nc2nc3cc(C(N)=O)cc(OC)c3n2C/C=C/Cn2c(NC(=O)c3oc(CC)nc3C)nc3cc(C(N)=O)cc(OCCCN4CCC(c5nnc6n5CCNC6)CC4)c32)o1] |
| 47 | 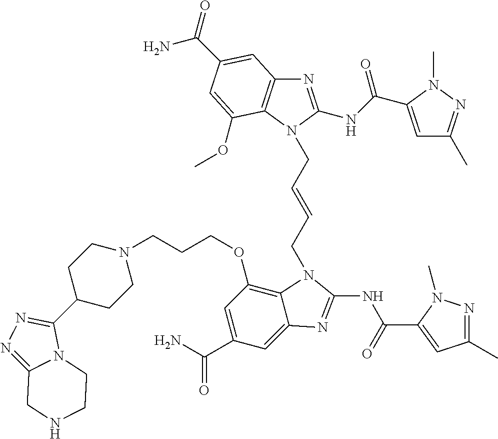 [SMILES: COc1cc(C(N)=O)cc2nc(NC(=O)c3cc(C)nn3C)n(C/C=C/Cn3c(NC(=O)c4cc(C)nn4C)nc4cc(C(N)=O)cc(OCCCN5CCC(c6nnc7n6CCNC7)CC5)c43)c12] |
| 48 | 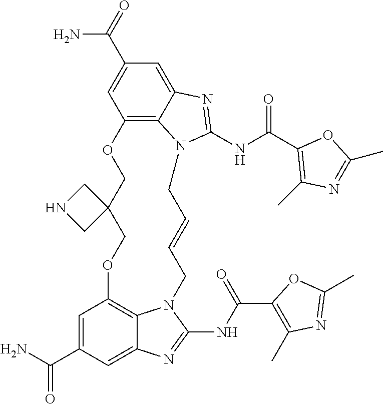 [SMILES: Cc1nc(C)c(C(=O)Nc2nc3cc(C(N)=O)cc4c3n2C/C=C/Cn2c(NC(=O)c3oc(C)nc3C)nc3cc(C(N)=O)cc(c32)OCC2(CNC2)CO4)o1] |
| 49 | 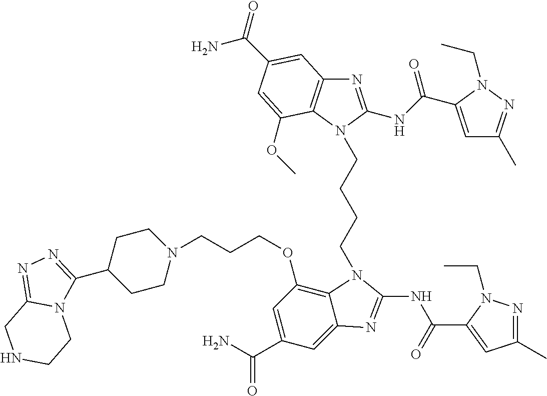 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCNC5)CC3)c21] |
| 50 | 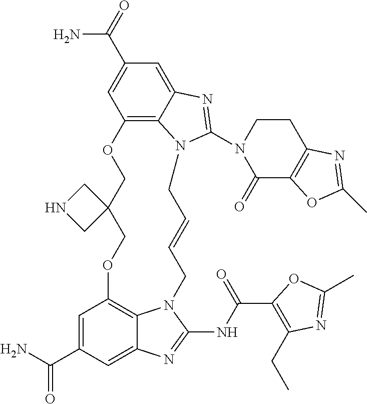 [SMILES: CCc1nc(C)oc1C(=O)Nc1nc2cc(C(N)=O)cc3c2n1C/C=C/Cn1c(N2CCc4nc(C)oc4C2=O)nc2cc(C(N)=O)cc(c21)OCC1(CNC1)CO3] |
| 51 | 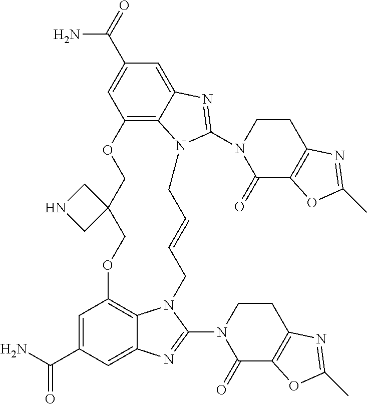 [SMILES: Cc1nc2c(o1)C(=O)N(c1nc3cc(C(N)=O)cc4c3n1C/C=C/Cn1c(N3CCc5nc(C)oc5C3=O)nc3cc(C(N)=O)cc(c31)OCC1(CNC1)CO4)CC2] |
| 52 | 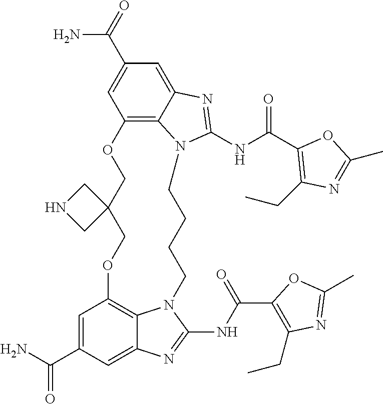 [SMILES: CCc1nc(C)oc1C(=O)Nc1nc2cc(C(N)=O)cc3c2n1CCCCn1c(NC(=O)c2oc(C)nc2CC)nc2cc(C(N)=O)cc(c21)OCC1(CNC1)CO3] |
| 54 | 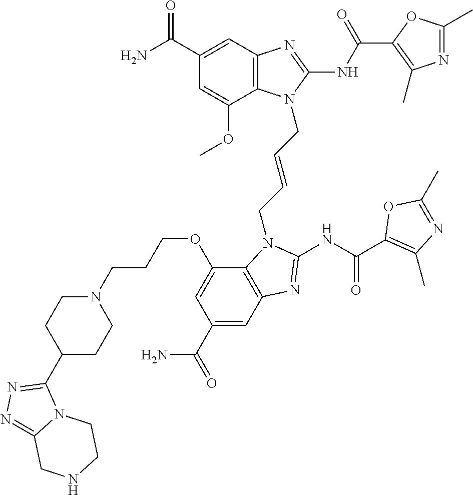 [SMILES: COc1cc(C(N)=O)cc2nc(NC(=O)c3oc(C)nc3C)n(C/C=C/Cn3c(NC(=O)c4oc(C)nc4C)nc4cc(C(N)=O)cc(OCCCN5CCC(c6nnc7n6CCNC7)CC5)c43)c12] |
| 56 | 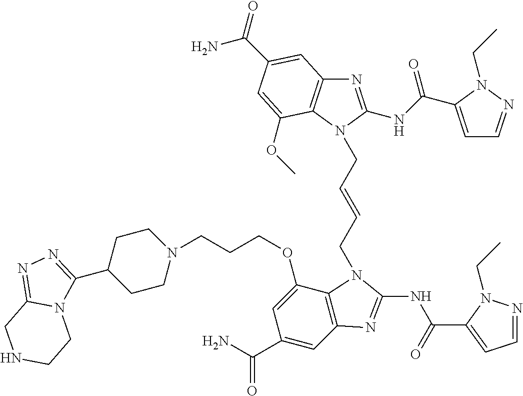 [SMILES: CCn1nccc1C(=O)Nc1nc2cc(C(N)=O)cc(OC)c2n1C/C=C/Cn1c(NC(=O)c2ccnn2CC)nc2cc(C(N)=O)cc(OCCCN3CCC(c4nnc5n4CCNC5)CC3)c21] |
| 57 | 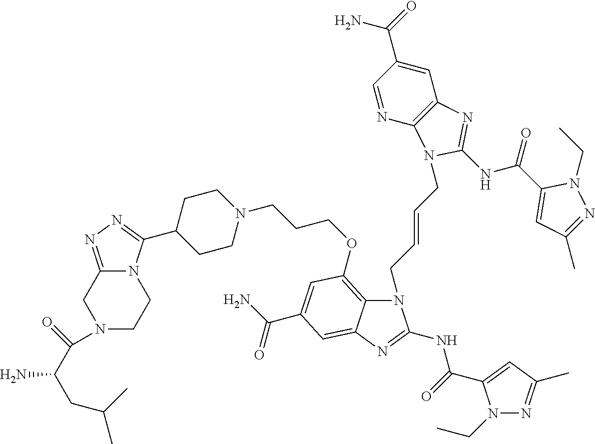 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)[C@@H](N)CC(C)C)C5)CC3)c21] |
| 58 | 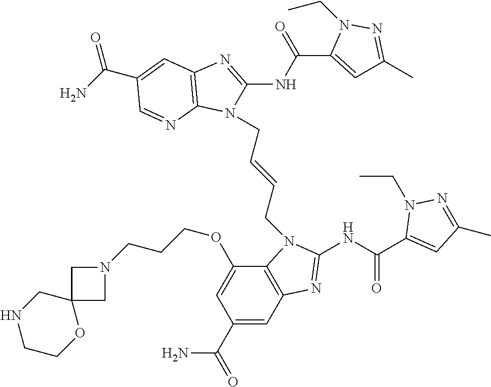 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CC4(CNCCO4)C3)c21] |
| 59 | 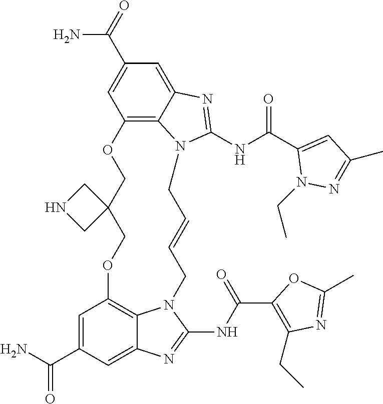 [SMILES: CCc1nc(C)oc1C(=O)Nc1nc2cc(C(N)=O)cc3c2n1C/C=C/Cn1c(NC(=O)c2cc(C)nn2CC)nc2cc(C(N)=O)cc(c21)OCC1(CNC1)CO3] |
| 60 | 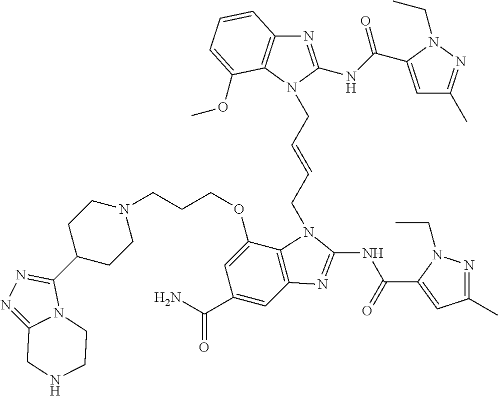 [SMILES: CCn1nc(C)cc1C(=O)Nc1nc2cccc(OC)c2n1C/C=C/Cn1c(NC(=O)c2cc(C)nn2CC)nc2cc(C(N)=O)cc(OCCCN3CCC(c4nnc5n4CCNC5)CC3)c21] |
| 61 | 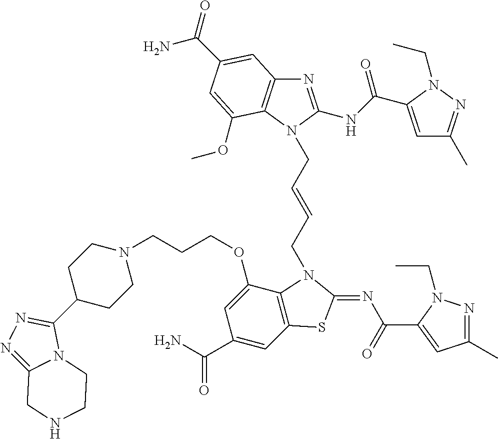 [SMILES: CCn1nc(C)cc1C(=O)/N=c1\sc2cc(C(N)=O)cc(OCCCN3CCC(c4nnc5n4CCNC5)CC3)c2n1C/C=C/Cn1c(NC(=O)c2cc(C)nn2CC)nc2cc(C(N)=O)cc(OC)c21] |
| 62 | 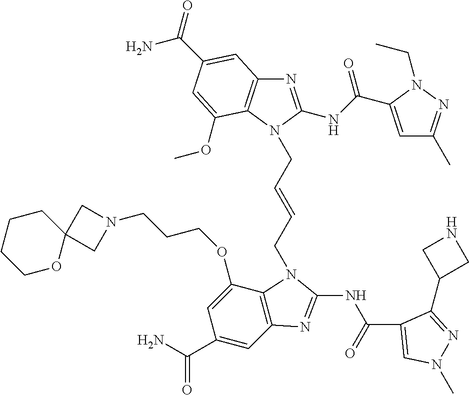 [SMILES: CCn1nc(C)cc1C(=O)Nc1nc2cc(C(N)=O)cc(OC)c2n1C/C=C/Cn1c(NC(=O)c2cn(C)nc2C2CNC2)nc2cc(C(N)=O)cc(OCCCN3CC4(CCCCO4)C3)c21] |
| 63 | 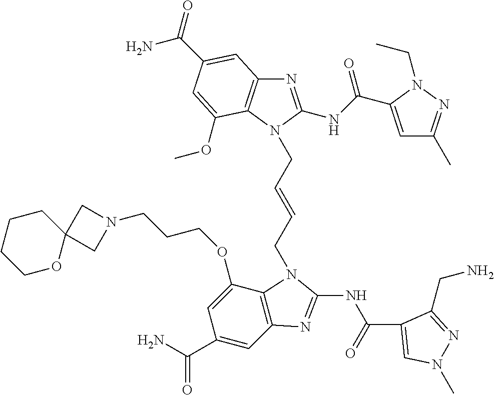 [SMILES: CCn1nc(C)cc1C(=O)Nc1nc2cc(C(N)=O)cc(OC)c2n1C/C=C/Cn1c(NC(=O)c2cn(C)nc2CN)nc2cc(C(N)=O)cc(OCCCN3CC4(CCCCO4)C3)c21] |
| 64 | 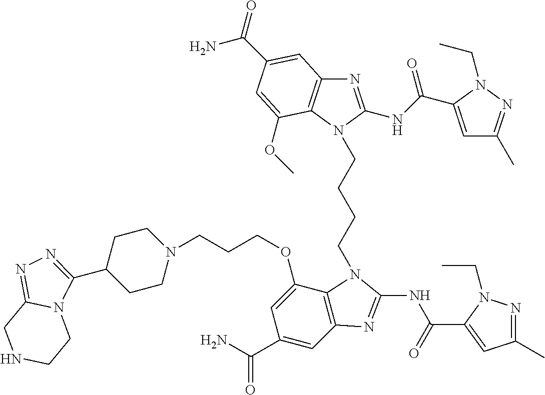 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCNC5)CC3)c21] |
| 65 | 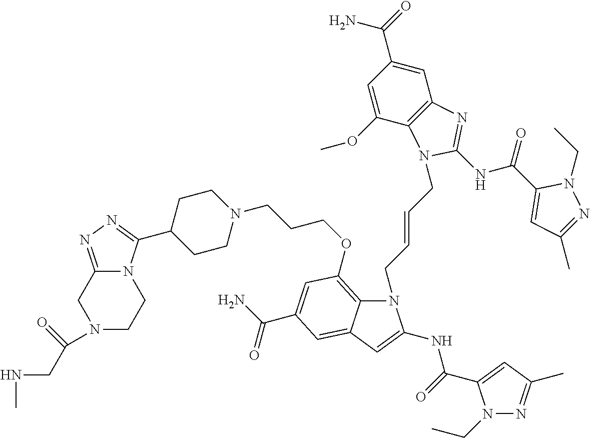 [SMILES: CCn1nc(C)cc1C(=O)Nc1cc2cc(C(N)=O)cc(OCCCN3CCC(c4nnc5n4CCN(C(=O)CNC)C5)CC3)c2n1C/C=C/Cn1c(NC(=O)c2cc(C)nn2CC)nc2cc(C(N)=O)cc(OC)c21] |
| 66 | 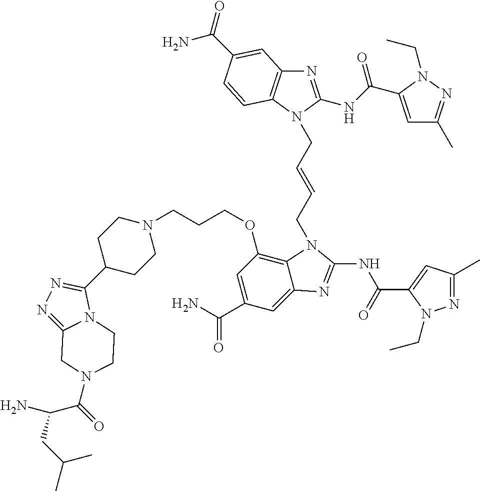 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)[C@@H](N)CC(C)C)C5)CC3)c21] |

TABLE A-1

| Compound | |
|---|---|
| No. | Structure |
| 67 | 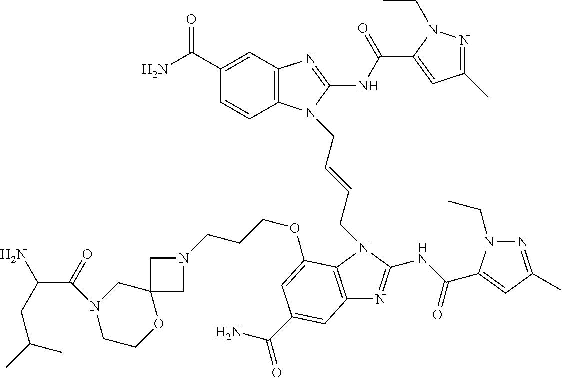 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)C(N)CC(C)C)CCO4)c21] |
| 68 | 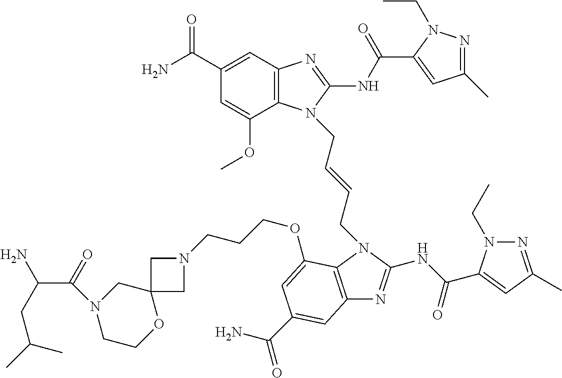 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(C3)CN(C(=O)C(N)CC(C)C)CCO4)c21] |
| 69 | 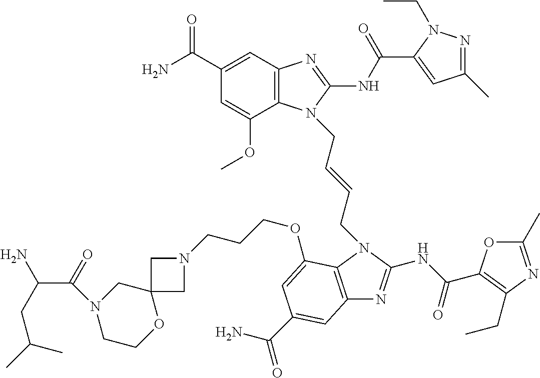 [SMILES: CCc1nc(C)oc1C(=O)Nc1nc2cc(C(N)=O)cc(OCCCN3CC4(C3)CN(C(=O)C(N)CC(C)C)CCO4)c2n1C/C=C/Cn1c(NC(=O)c2cc(C)nn2CC)nc2cc(C(N)=O)cc(OC)c21] |
| 70 | 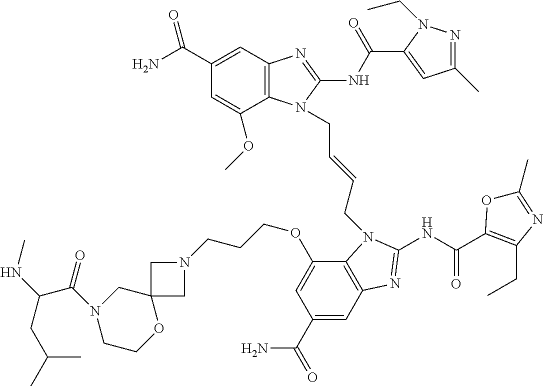 [SMILES: CCc1nc(C)oc1C(=O)Nc1nc2cc(C(N)=O)cc(OCCCN3CC4(C3)CN(C(=O)C(CC(C)C)NC)CCO4)c2n1C/C=C/Cn1c(NC(=O)c2cc(C)nn2CC)nc2cc(C(N)=O)cc(OC)c21] |
| 71 | 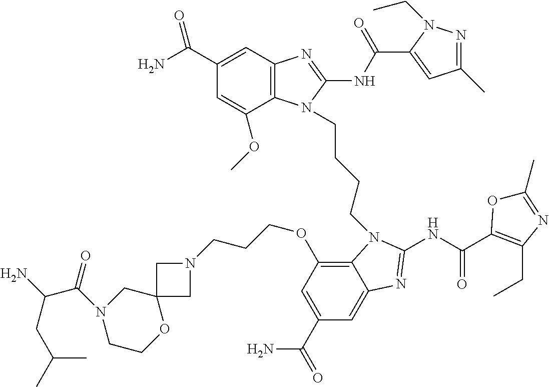 [SMILES: CCc1nc(C)oc1C(=O)Nc1nc2cc(C(N)=O)cc(OCCCN3CC4(C3)CN(C(=O)C(N)CC(C)C)CCO4)c2n1CCCCn1c(NC(=O)c2cc(C)nn2CC)nc2cc(C(N)=O)cc(OC)c21] |
| 72 | 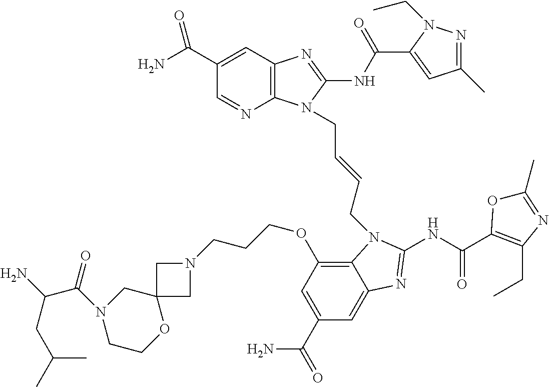 [SMILES: CCc1nc(C)oc1C(=O)Nc1nc2cc(C(N)=O)cc(OCCCN3CC4(C3)CN(C(=O)C(N)CC(C)C)CCO4)c2n1C/C=C/Cn1c(NC(=O)c2cc(C)nn2CC)nc2cc(C(N)=O)cnc21] |
| 73 | 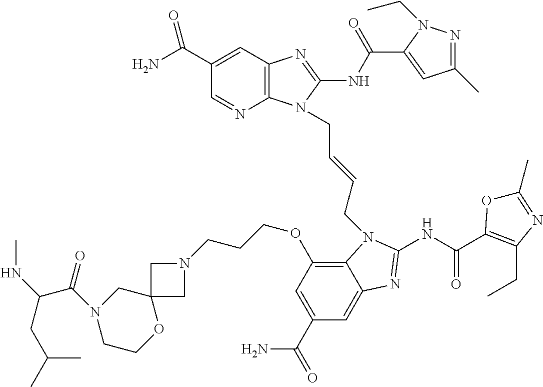 [SMILES: CCc1nc(C)oc1C(=O)Nc1nc2cc(C(N)=O)cc(OCCCN3CC4(C3)CN(C(=O)C(CC(C)C)NC)CCO4)c2n1C/C=C/Cn1c(NC(=O)c2cc(C)nn2CC)nc2cc(C(N)=O)cnc21] |
| 74 | 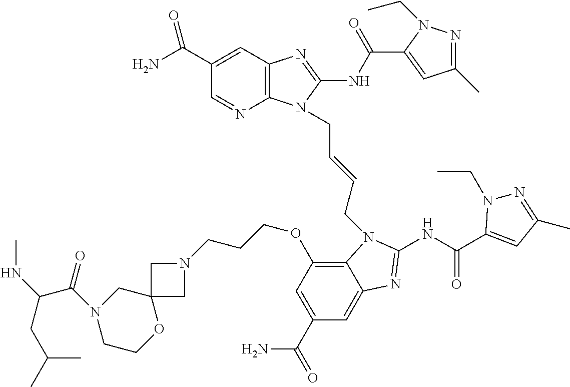 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CC4(C3)CN(C(=O)C(CC(C)C)NC)CCO4)c21] |
| 75 | 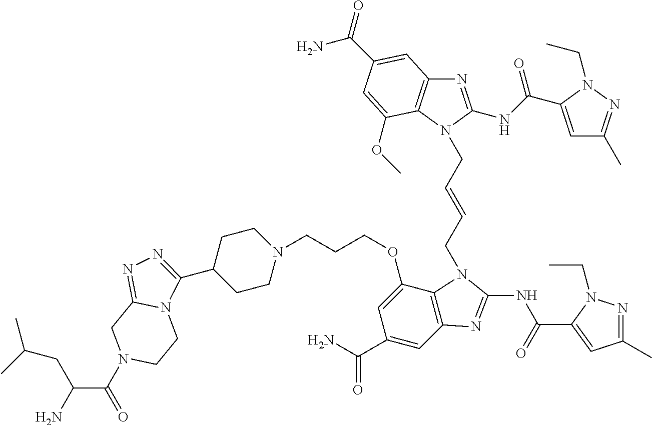 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)C(N)CC(C)C)C5)CC3)c21] |
| 76 | 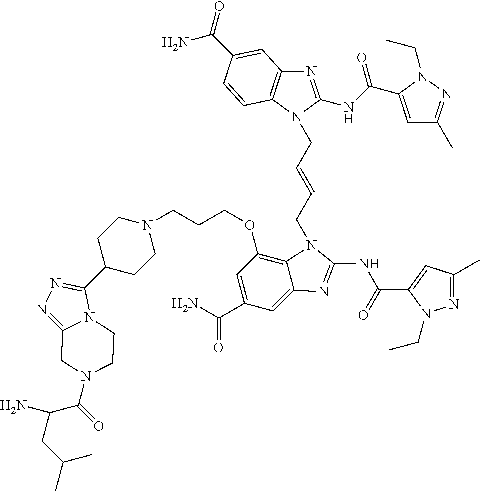 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)C(N)CC(C)C)C5)CC3)c21] |

TABLE A-2

| Compound | |
|---|---|
| No. | Structure |
| 77 | 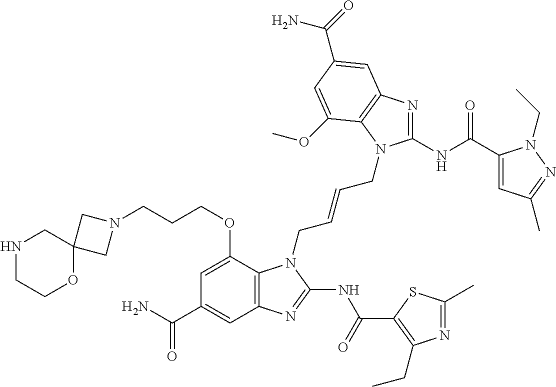 [SMILES: CCc1nc(C)sc1C(=O)Nc1nc2cc(C(N)=O)cc(OCCCN3CC4(CNCCO4)C3)c2n1C/C=C/Cn1c(NC(=O)c2cc(C)nn2CC)nc2cc(C(N)=O)cc(OC)c21] |
| 78 | 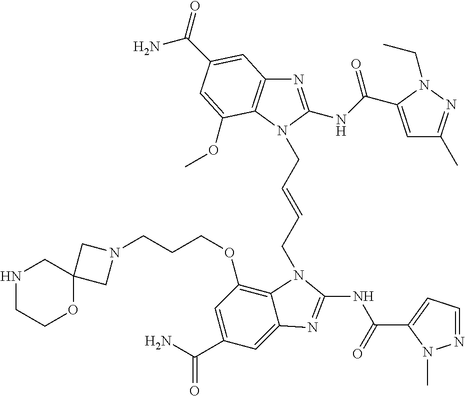 [SMILES: CCn1nc(C)cc1C(=O)Nc1nc2cc(C(N)=O)cc(OC)c2n1C/C=C/Cn1c(NC(=O)c2ccnn2C)nc2cc(C(N)=O)cc(OCCCN3CC4(CNCCO4)C3)c21] |
| 79 | 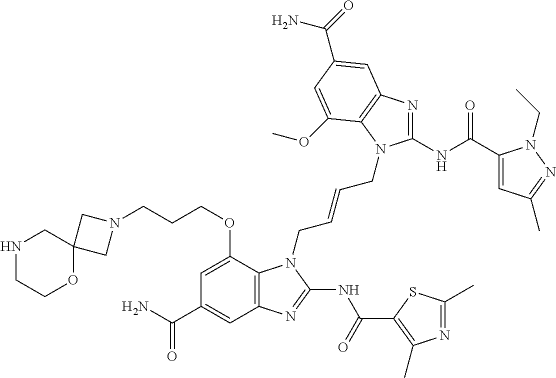 [SMILES: CCn1nc(C)cc1C(=O)Nc1nc2cc(C(N)=O)cc(OC)c2n1C/C=C/Cn1c(NC(=O)c2sc(C)nc2C)nc2cc(C(N)=O)cc(OCCCN3CC4(CNCCO4)C3)c21] |
| 80 | 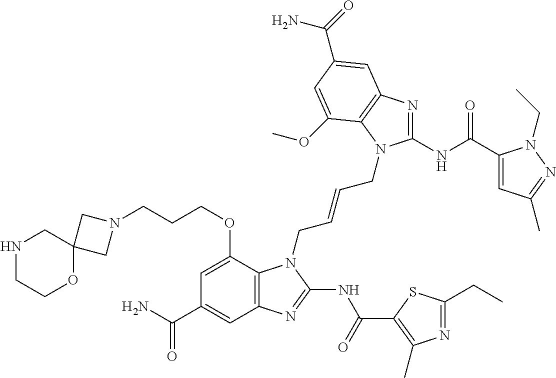 [SMILES: CCc1nc(C)c(C(=O)Nc2nc3cc(C(N)=O)cc(OCCCN4CC5(CNCCO5)C4)c3n2C/C=C/Cn2c(NC(=O)c3cc(C)nn3CC)nc3cc(C(N)=O)cc(OC)c32)s1] |
| 81 | 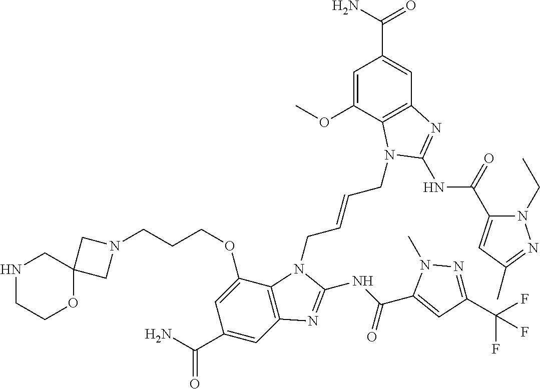 [SMILES: CCn1nc(C)cc1C(=O)Nc1nc2cc(C(N)=O)cc(OC)c2n1C/C=C/Cn1c(NC(=O)c2cc(C(F)(F)F)nn2C)nc2cc(C(N)=O)cc(OCCCN3CC4(CNCCO4)C3)c21] |
| 82 | 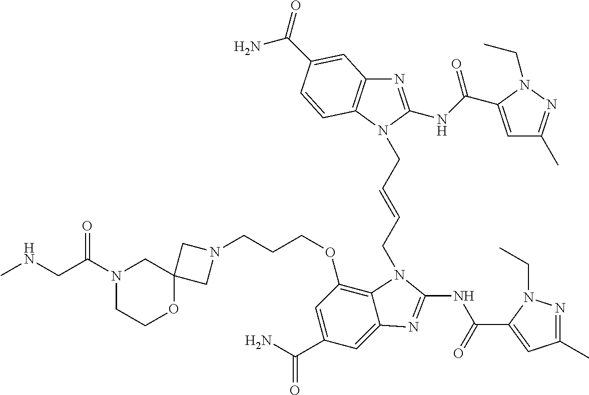 |
| 83 | 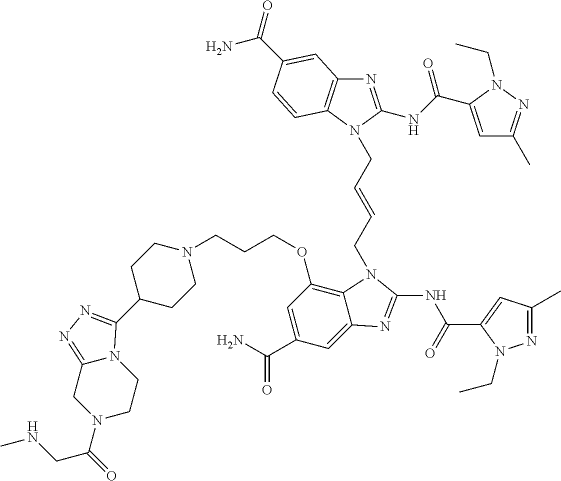 |
| 84 | 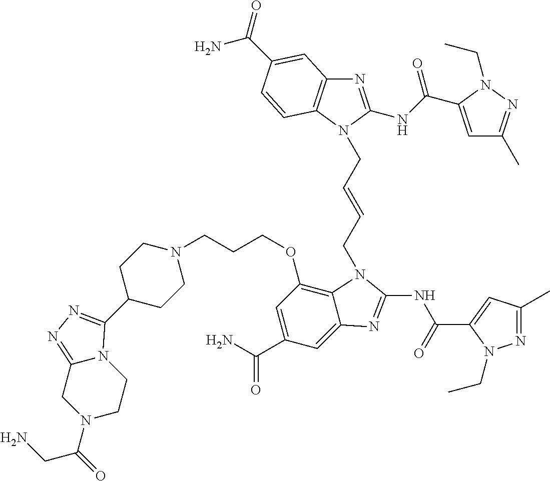 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)CN)C5)CC3)c21] |
| 85 | 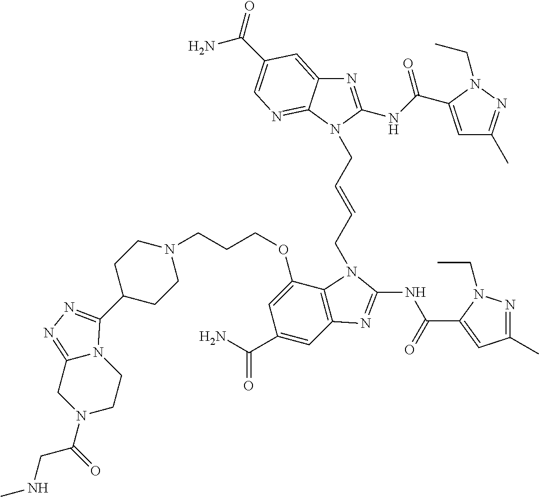 |
| 86 | 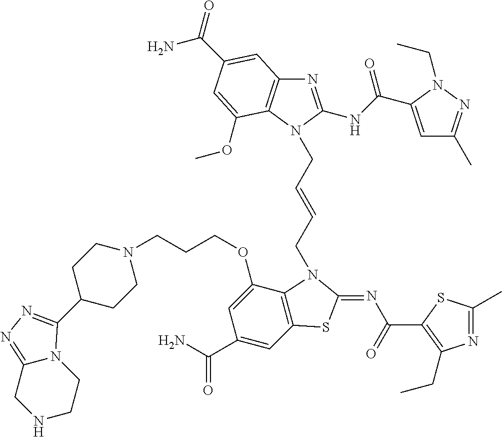 [SMILES: CCc1nc(C)sc1C(=O)/N=c1\sc2cc(C(N)=O)cc(OCCCN3CCC(c4nnc5n4CCNC5)CC3)c2n1C/C=C/Cn1c(NC(=O)c2cc(C)nn2CC)nc2cc(C(N)=O)cc(OC)c21] |
| 87 | 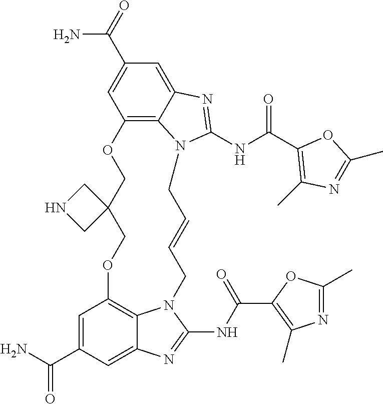 [SMILES: Cc1nc(C)c(C(=O)Nc2nc3cc(C(N)=O)cc4c3n2C/C=C/Cn2c(NC(=O)c3oc(C)nc3C)nc3cc(C(N)=O)cc(c32)OCC2(CNC2)CO4)o1] |
| 88 | 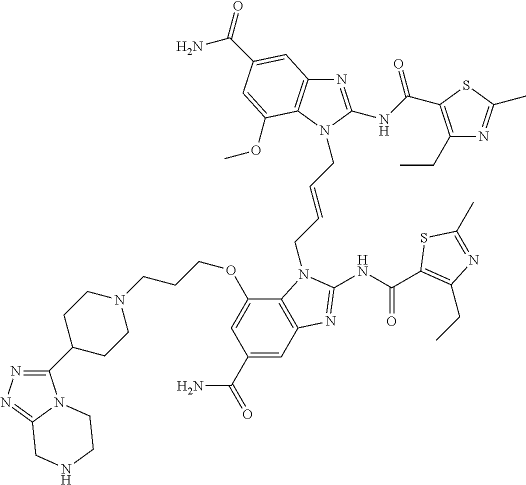 [SMILES: CCc1nc(C)sc1C(=O)Nc1nc2cc(C(N)=O)cc(OC)c2n1C/C=C/Cn1c(NC(=O)c2sc(C)nc2CC)nc2cc(C(N)=O)cc(OCCCN3CCC(c4nnc5n4CCNC5)CC3)c21] |
| 89 | 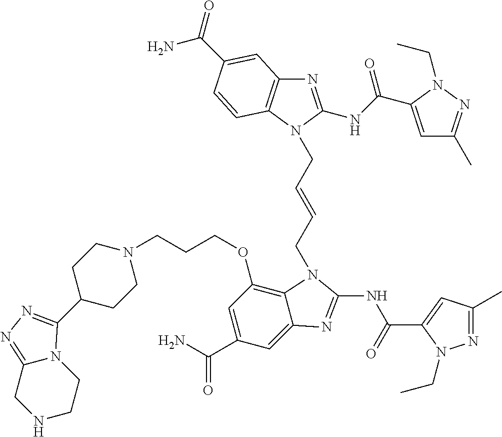 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCNC5)CC3)c21] |
| 90 | 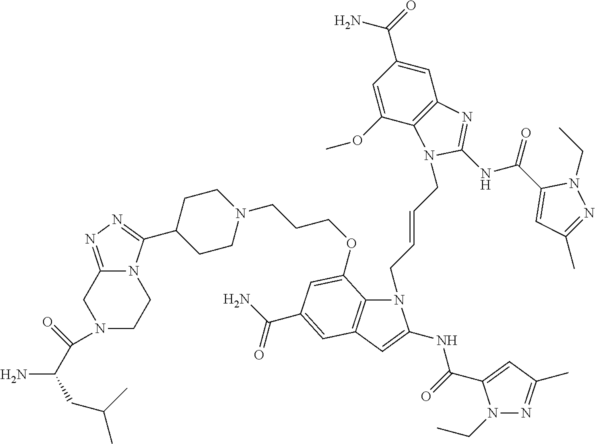 [SMILES: CCn1nc(C)cc1C(=O)Nc1cc2cc(C(N)=O)cc(OCCCN3CCC(c4nnc5n4CCN(C(=O)[C@@H](N)CC(C)C)C5)CC3)c2n1C/C=C/Cn1c(NC(=O)c2cc(C)nn2CC)nc2cc(C(N)=O)cc(OC)c21] |

b. Linker Payloads of Formula (LP-I), (LP-II), (LP-III), (LP-IV), and (LP-V)

Provided herein are linker-payload compounds comprising a compound of Formula (I), (II) (III), or (IV) wherein the compound of Formula (I), (II), (III) or (IV) is linked to RG optionally via a linker wherein RG is a reactive linker group.

In one embodiment, the compound conjugate is of Formula (LP-IA) or Formula (LP—IB).

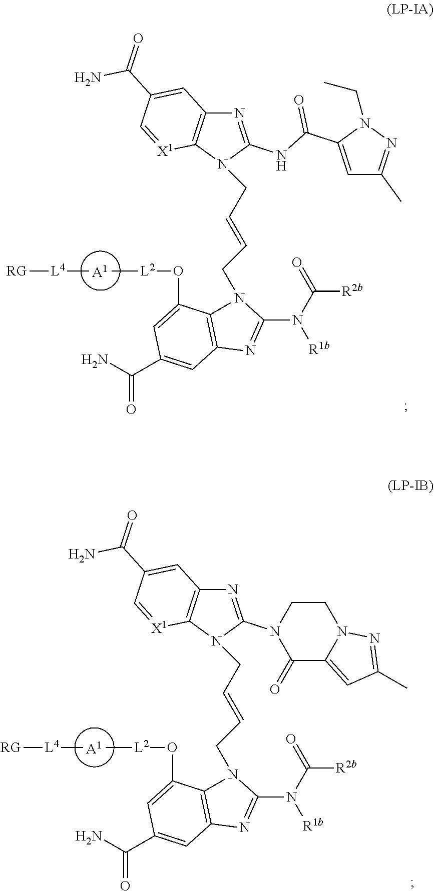

or a pharmaceutically acceptable salt or tautomer thereof.

In one embodiment of Formula (LP-IA), L² is —(CH₂)₃—; R^{1b} is hydrogen; and R^{2b} is

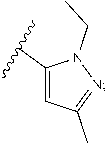 [SMILES: *c1cc(C)nn1CC]

In one embodiment of Formula (LP-IA), L² is —(CH₂)₃—; and R^{1b} and R^{2b} are joined together to form

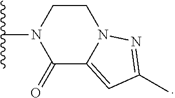 [SMILES: *N1CCn2nc(C)cc2C1=O]

In one embodiment of Formula (LP-IA), L² is —(CH₂)₃—; R^{1b} is hydrogen; and R^{2b} is

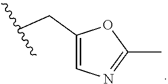 [SMILES: *Cc1cnc(C)o1]

In one embodiment of Formula (LP-IA), L² is —(CH₂)₃—; R^{1b} is hydrogen; and R^{2b} is

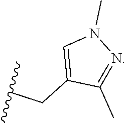 [SMILES: *Cc1cn(C)nc1C]

In one embodiment of Formula (LP-IA), L² is —(CH₂)₃—; R^{1b} is hydrogen; and R^{2b} is

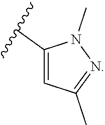 [SMILES: *c1cc(C)nn1C]

In one embodiment of Formula (LP-IA), L² is —(CH₂)₃—; R^{1b} is hydrogen; and R^{2b} is

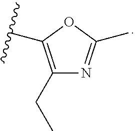 [SMILES: *c1oc(C)nc1CC]

In one embodiment of Formula (LP-IA), L² is —(CH₂)₃—; R^{1b} is hydrogen; and R^{2b} is

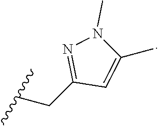 [SMILES: *Cc1cc(C)n(C)n1]

In one embodiment of Formula (LP-IA), L² is —(CH₂)₃—; R^{1b} is hydrogen; and R^{2b} is

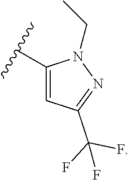 [SMILES: *c1cc(C(F)(F)F)nn1CC]

In one embodiment of Formula (LP-IA), L² is —(CH₂)₃—; R^{1b} is hydrogen; and R^{2b} is

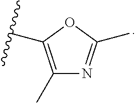 [SMILES: *c1oc(C)nc1C]

In one embodiment of Formula (LP-IA), L² is —(CH₂)₃—; R^{1b} is hydrogen; and R^{2b} is

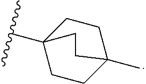 [SMILES: *C12CCC(C)(CC1)CC2]

In one embodiment of Formula (LP-IA), L² is —(CH₂)₃—; R^{1b} is hydrogen; and R^{2b} is

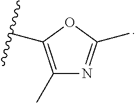 [SMILES: *c1oc(C)nc1C]

In one embodiment of Formula (LP-IA), L² is —(CH₂)₃—; R^{1b} is hydrogen; and R^{2b} is

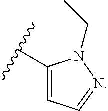 [SMILES: *c1ccnn1CC]

In one embodiment of Formula (LP-IA), L² is —(CH₂)₃—; R^{1b} is hydrogen; and R^{2b} is

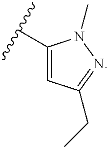 [SMILES: *c1cc(CC)nn1C]

In one embodiment of Formula (LP-IA), L² is —(CH₂)₃—; R^{1b} is hydrogen; and R^{2b} is

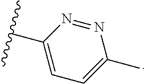 [SMILES: *c1ccc(C)nn1]

In one embodiment of Formula (LP-IA), L² is —(CH₂)₃—; R^{1b} is hydrogen; and R^{2b} is

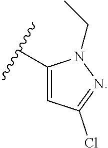 [SMILES: *c1cc(Cl)nn1CC]

In one embodiment of Formula (LP-IA), L² is —(CH₂)₃—; R^{1b} is hydrogen; and R^{2b} is

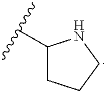 [SMILES: *C1CCCN1]

In one embodiment of Formula (LP-IA), L² is —(CH₂)₃—; R^{1b} is hydrogen; and R^{2b} is

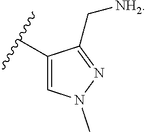 [SMILES: *c1cn(C)nc1CN]

In one embodiment of Formula (LP-IA), L² is —(CH₂)₃—; R^{1b} is hydrogen; and R^{2b} is

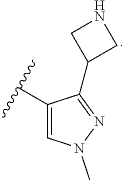 [SMILES: *c1cn(C)nc1C1CNC1]

In one embodiment of Formula (LP-IB), L² is —(CH₂)₃—; and R^{1b} and R^{2b} are joined together to form

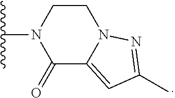 [SMILES: *N1CCn2nc(C)cc2C1=O]

In one embodiment of Formula (LP-IB), L² is —(CH₂)₃—; and R^{1b} and R^{2b} are joined together to form

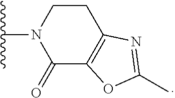 [SMILES: *N1CCc2nc(C)oc2C1=O]

In one embodiment, the compound conjugate is of Formula (LP-IIA), (LP-IIB), or (LP-IIC):

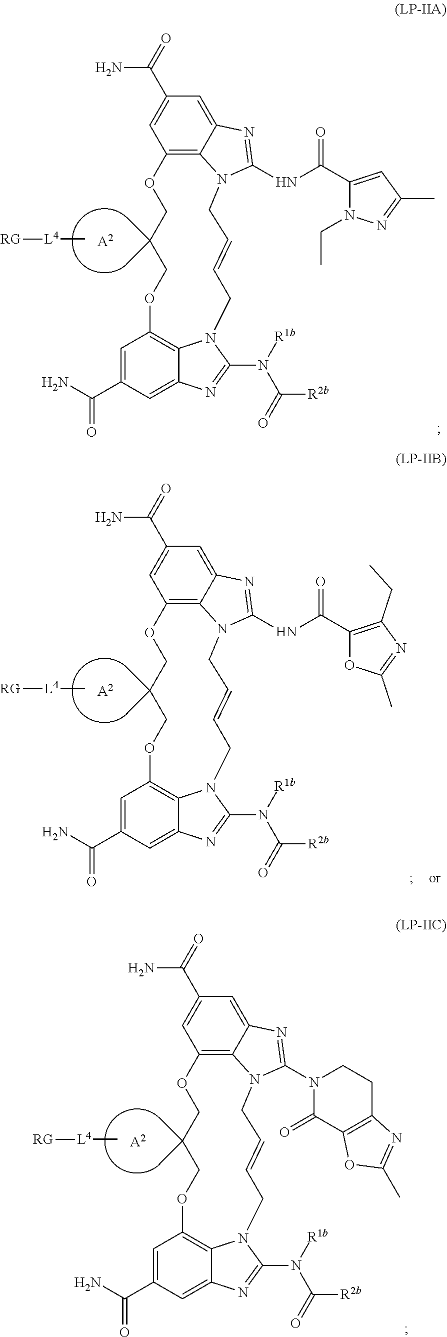

or a pharmaceutically acceptable salt or tautomer thereof, wherein

Ring A² is an optionally substituted C₃₋₁₂ carbocycle or an optionally substituted 3- to 12-membered heterocycle, wherein said optionally substituted C₃₋₁₂ carbocycle and optionally substituted 3- to 12-membered heterocycle are optionally substituted with one or more R⁵².

In one embodiment of Formula (LP-IIA), R^{1b} is hydrogen; and R^{2b} is

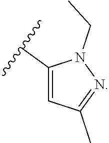 [SMILES: *c1cc(C)nn1CC]

In one embodiment of Formula (LP-IIA), and R^{1b} and R^{2b} are joined together to form

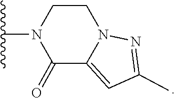 [SMILES: *N1CCn2nc(C)cc2C1=O]

In one embodiment of Formula (LP-IIB), R^{1b} is hydrogen; and R^{2b} is

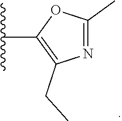 [SMILES: *c1oc(C)nc1CC]

In one embodiment of Formula (LP-IIB), R^{1b} is hydrogen; and R^{2b} is

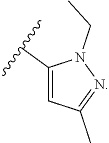 [SMILES: *c1cc(C)nn1CC]

In one embodiment of Formula (LP-IIC), R^{1b} is hydrogen; and R^{2b} is

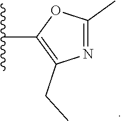 [SMILES: *c1oc(C)nc1CC]

In one embodiment of Formula (LP-IIC), and R^{1b} and R^{2b} are joined together to form

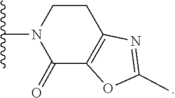 [SMILES: *N1CCc2nc(C)oc2C1=O]

In one embodiment, the compound conjugate is of Formula (LP-IIIA):

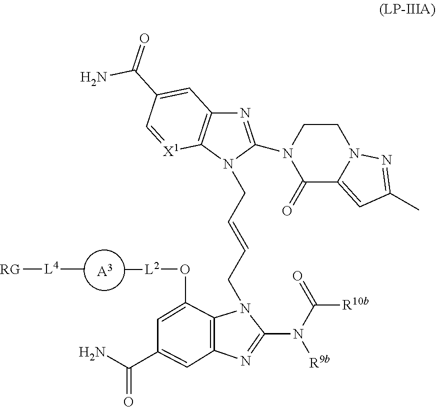

or a pharmaceutically acceptable salt or tautomer thereof.

In one embodiment of Formula (LP-IIIA), L² is —(CH₂)₃— and R^{10b} and R^{10a} are joined together to form

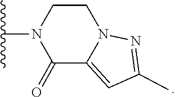 [SMILES: *N1CCn2nc(C)cc2C1=O]

In one embodiment, the compound conjugate is of Formula (LP-IVA):

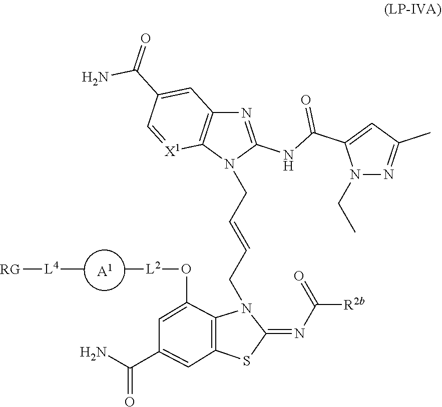

or a pharmaceutically acceptable salt or tautomer thereof.

In one embodiment of Formula (LP-IVA), L² is —(CH₂)₃— and R^{2b} is

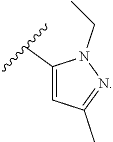 [SMILES: *c1cc(C)nn1CC]

In one embodiment, the compound conjugate is of Formula (LP-VA):

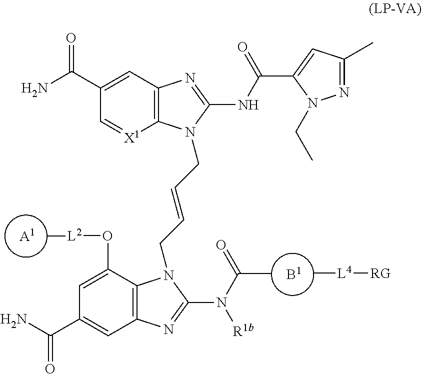

or a pharmaceutically acceptable salt or tautomer thereof.

In one embodiment of Formula (LP-VA)

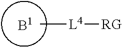

is

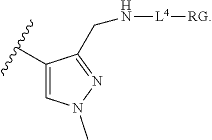

In one embodiment of Formula (LP-VA),

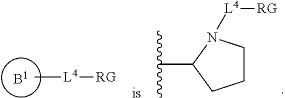

In one embodiment of Formula (LP-I), L¹ is —CH═CH—; R^{1a} and R^{1b} are both hydrogen; R^{2a} is

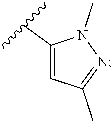 [SMILES: *c1cc(C)nn1C]

### and R^{2b} is

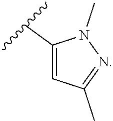 [SMILES: *c1cc(C)nn1C]

In one embodiment of Formula (LP-I), L¹ is —CH═CH—; R^{1a} and R^{1b} are both hydrogen; R^{2a} is

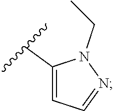 [SMILES: *c1ccnn1CC]

### and R^{2b} is

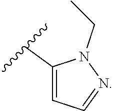 [SMILES: *c1ccnn1CC]

In one embodiment of Formula (LP-I), L¹ is —CH═CH—; R^{1a} and R^{1b} are both hydrogen; R^{2a} is

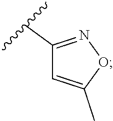 [SMILES: *c1cc(C)on1]

### and R^{2b} is

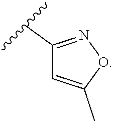 [SMILES: *c1cc(C)on1]

In one embodiment of Formula (LP-I), L¹ is —CH═CH—; R^{1a} and R^{1b} are both hydrogen; R^{2a} is

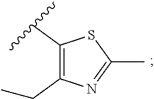 [SMILES: *c1sc(C)nc1CC]

### and R^{2b} is

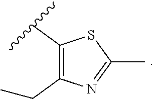 [SMILES: *c1sc(C)nc1CC]

In one embodiment of Formula (LP-I), L¹ is —CH═CH—; R^{1a} and R^{1b} are both hydrogen; R^{2a} is

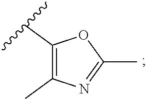 [SMILES: *c1oc(C)nc1C]

### and R^{2b} is

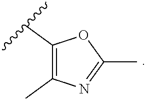 [SMILES: *c1oc(C)nc1C]

In one embodiment of Formula (LP-I), L¹ is —CH₂CH₂—; R^{1a} and R^{1b} are both hydrogen; R^{2a} is

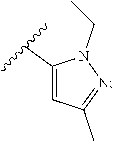 [SMILES: *c1cc(C)nn1CC]

### and R^{2b} is

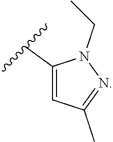 [SMILES: *c1cc(C)nn1CC]

In one embodiment of Formula (LP-I), L¹ is —CH═CH—; R²⁰ is hydrogen; R^{1a} and R^{1b} are both hydrogen; and R^{2a} and R^{2b} are both

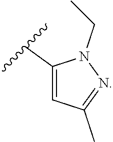 [SMILES: *c1cc(C)nn1CC]

In one embodiment of Formula (LP-II), L¹ is —CH═CH—; R^{1a} and R^{1b} are both hydrogen and R^{2a} and R^{2b} are both

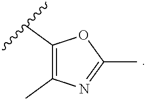 [SMILES: *c1oc(C)nc1C]

In one embodiment of Formula (LP-I), (LP-IA) (LP-IB), (LP-IV), (LP-IVA), (LP-V), or (LP-VA), including any of the foregoing, Ring A¹ is an optionally substituted bridged, fused, or spirocyclic bicyclic heterocycle comprising at least one N atom and at least one O atom wherein the heterocycle is optionally substituted with one or more R⁵³. In one embodiment of Formula (LP-I), (LP-IA) (LP-IB), (LP-IV), (LP-IVA), (LP-V), or (LP-VA), including any of the foregoing, Ring A¹ is an optionally substituted 3- to 12-membered bridged, fused, or spirocyclic bicyclic heterocycle comprising at least one nitrogen atom, including the nitrogen bound to L², and at least one oxygen atom. In one embodiment of Formula (LP-I), (LP-IA) (LP-IB), (LP-IV), (LP-IVA), (LP-V), or (LP-VA), including any of the foregoing, Ring A¹ is an optionally substituted 8- to 12-membered bridged, fused, or spirocyclic bicyclic heterocycle comprising at least one nitrogen atom, including the nitrogen bound to L², and at least one oxygen atom. In one embodiment of Formula (LP-I), (LP-IA) (LP-IB), (LP-IV), (LP-IVA), (LP-V), or (LP-VA), including any of the foregoing, Ring A¹ is an optionally substituted 3- to 12-membered N-linked bridged, fused, or spirocyclic bicyclic heterocycle comprising at least one nitrogen atom, including the nitrogen bound to L², and at least one oxygen atom. In one embodiment of Formula (LP-I), (LP-IA) (LP-IB), (LP-IV), (LP-IVA), (LP-V), or (LP-VA), including any of the foregoing, Ring A¹ is an optionally substituted 8- to 12-membered N-linked bridged, fused, or spirocyclic bicyclic heterocycle comprising at least one nitrogen atom, including the nitrogen bound to L², and at least one oxygen atom. In one embodiment of Formula (LP-I), (LP-IA) (LP-IB), (LP-IV), (LP-IVA), (LP-V), or (LP-VA), including any of the foregoing, Ring A¹ is an optionally substituted 3- to 12-membered N-linked spirocyclic bicyclic heterocycle comprising at least one nitrogen atom, including the nitrogen bound to L², and at least one oxygen atom. In one embodiment of Formula (LP-I), (LP-IA) (LP-IB), (LP-IV), (LP-IVA), (LP-V), or (LP-VA), including any of the foregoing, Ring A¹ is an optionally substituted 3- to 12-membered N-linked spirocyclic bicyclic heterocycle comprising two nitrogen atoms, including the nitrogen bound to L² and a nitrogen bound to L⁴, and one oxygen atom.

In one embodiment of Formula (LP-I), (LP-IA) (LP-IB), (LP-IV), (LP-IVA), (LP-V), or (LP-VA), including any of the foregoing, Ring A¹ is selected from

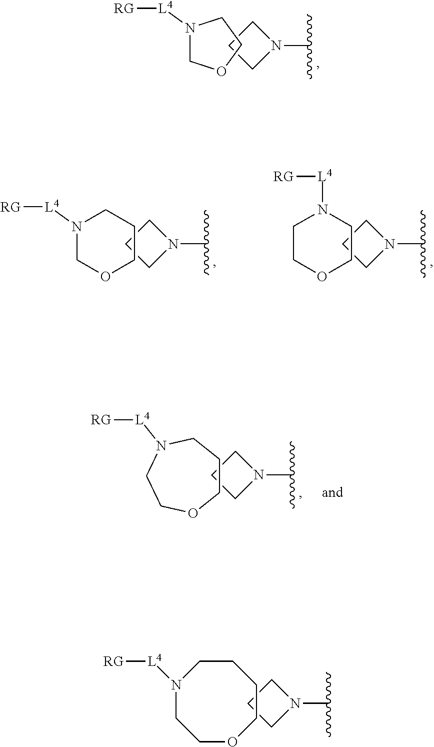

wherein L⁴, RG, and COMIP are as defined herein.

In one embodiment of Formula (LP-I), (LP-IA) (LP-IB), (LP-IV), (LP-IVA), (LP-V), or (LP-VA), including any of the foregoing, Ring A¹ is selected from

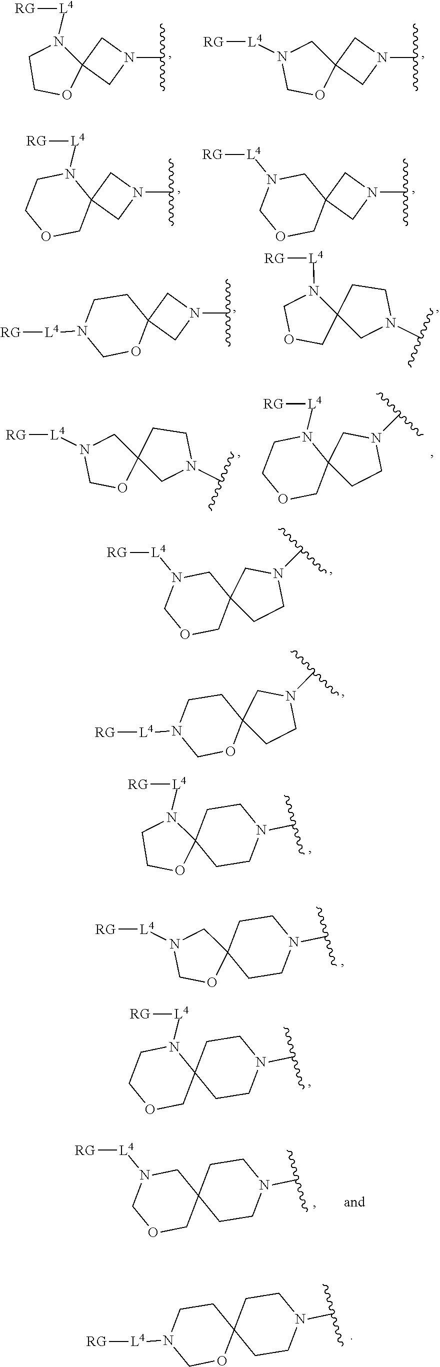

In one embodiment of Formula (LP-I), (LP-IA) (LP-IB), (LP-IV), (LP-IVA), (LP-V), or (LP-VA), including any of the foregoing, Ring A¹ is selected from

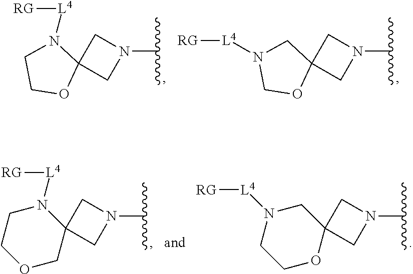

In one embodiment of Formula (LP-I), (LP-IA) (LP-IB), (LP-IV), (LP-IVA), (LP-V), or (LP-VA), including any of the foregoing, Ring A¹ is selected from

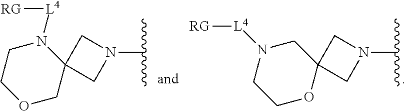

In one embodiment of Formula (LP-I), (LP-IA) (LP-IB), (LP-IV), (LP-IVA), (LP-V), or (LP-VA), including any of the foregoing, Ring A¹ is

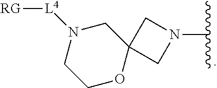

In one embodiment of Formula (LP-I), (LP-IA) (LP-IB), (LP-IV), (LP-IVA), (LP-V), or (LP-VA), including any of the foregoing, Ring A¹ is a 3- to 12-membered heterocycle substituted with R⁴. In one embodiment of Formula (LP-I), (LP-IA) (LP-IB), (LP-IV), (LP-IVA), (LP-V), or (LP-VA), including any of the foregoing, Ring A¹ is a 8- to 12-membered heterocycle substituted with R⁴.

In one embodiment of Formula (LP-I), (LP-IA) (LP-IB), (LP-IV), (LP-IVA), (LP-V), or (LP-VA), including any of the foregoing, Ring A¹ is a N-linked monocyclic 3- to 12-membered heterocycle comprising the N to which the ring is attached wherein the heterocycle is substituted with R⁴. In one embodiment of Formula (LP-I), (LP-IA) (LP-IB), (LP-IV), (LP-IVA), (LP-V), or (LP-VA), including any of the foregoing, Ring A¹ is a N-linked monocyclic 8- to 12-membered heterocycle comprising the N to which the ring is attached and a second NH and wherein the heterocycle is substituted with R⁴.

In one embodiment of Formula (LP-I), (LP-IA) (LP-IB), (LP-IV), (LP-IVA), (LP-V), or (LP-VA), including any of the foregoing, Ring A¹ is selected from

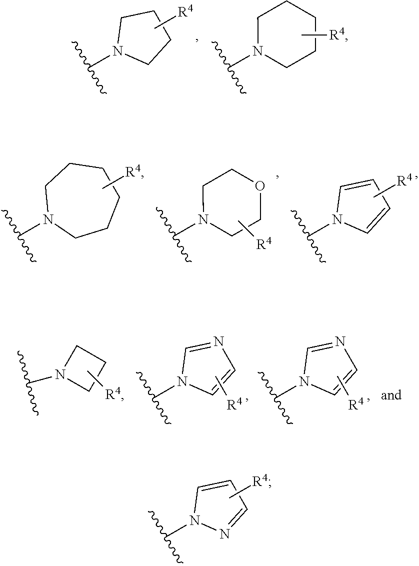

wherein

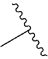

is the point of attachment to the rest of the compound.

In one embodiment of Formula (LP-I), (LP-IA) (LP-IB), (LP-IV), (LP-IVA), (LP-V), or (LP-VA), including any of the foregoing, Ring A¹ is selected from

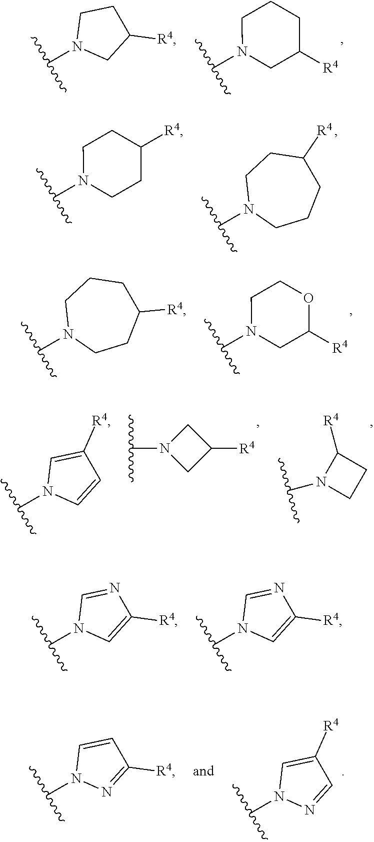

In one embodiment of Formula (LP-I), (LP-IA) (LP-IB), (LP-IV), (LP-IVA), (LP-V), or (LP-VA), including any of the foregoing, Ring A¹ is selected from

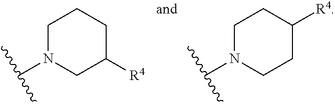

In one embodiment of Formula (LP-I), (LP-IA) (LP-IB) (LP-IV), (LP-IVA), (LP-V), or (LP-VA), including any of the foregoing, Ring A¹ is

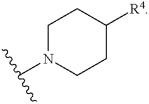

In one embodiment of Formula (LP-I), (LP-IA) (LP-IB), (LP-IV), (LP-IVA), (LP-V), or (LP-VA), including any of the foregoing, R⁴ is a 3- to 12-membered bridged or fused bicyclic heterocycle comprising 1, 2, 3, or 4 heteroatoms selected from N, S, and O. In one embodiment of Formula (LP-I), (LP-IA) (LP-IB), (LP-IV), (LP-IVA), (LP-V), or (LP-VA), including any of the foregoing, R⁴ is a 8- to 12-membered bridged or fused bicyclic heterocycle comprising 1, 2, 3, or 4 heteroatoms selected from N, S, and O. In one embodiment of Formula (LP-I), (LP-IA) (LP-IB), (LP-IV), (LP-IVA), (LP-V), or (LP-VA), including any of the foregoing, R⁴ is a 5-5 fused ring system, a 5-6 fused ring system, a 6-6 fused ring system, or a 5-7 fused ring system comprising 1, 2, 3, or 4 heteroatoms selected from N, S, and O. In one embodiment of Formula (LP-I), (LP-IA) (LP-IB), (LP-IV), (LP-IVA), (LP-V), or (LP-VA), including any of the foregoing, R⁴ is 5-6 fused ring system comprising 1, 2, 3, or 4 heteroatoms selected from N, S, and O.

In one embodiment of Formula (LP-I), (LP-IA) (LP-IB), (LP-IV), or (LP-IVA), including any of the foregoing, R⁴ is a 3- to 12-membered bridged or fused bicyclic heterocycle comprising 1, 2, 3, or 4 heteroatoms selected from N, S, and O wherein at least one heteroatom is a N bound to -L⁴-RG. In one embodiment of Formula (LP-I), (LP-IA) (LP-IB), (LP-IV), or (LP-IVA), including any of the foregoing, R⁴ is a 5-5 fused ring system, a 5-6 fused ring system, a 6-6 fused ring system, or a 5-7 fused ring system comprising 1, 2, 3, or 4 heteroatoms selected from N, S, and O wherein at least one heteroatom is a N bound to -L⁴-RG. In one embodiment of Formula (LP-I), (LP-IA) (LP-IB), (LP-IV), or (LP-IVA), including any of the foregoing, R⁴ is a 5-6 fused ring system comprising 1, 2, 3, or 4 heteroatoms selected from N, S, and O wherein at least one heteroatom is a N bound to -L⁴-RG.

In one embodiment of Formula (LP-I), (LP-IA) (LP-IB), (LP-IV), or (LP-IVA), including any of the foregoing, R⁴ is

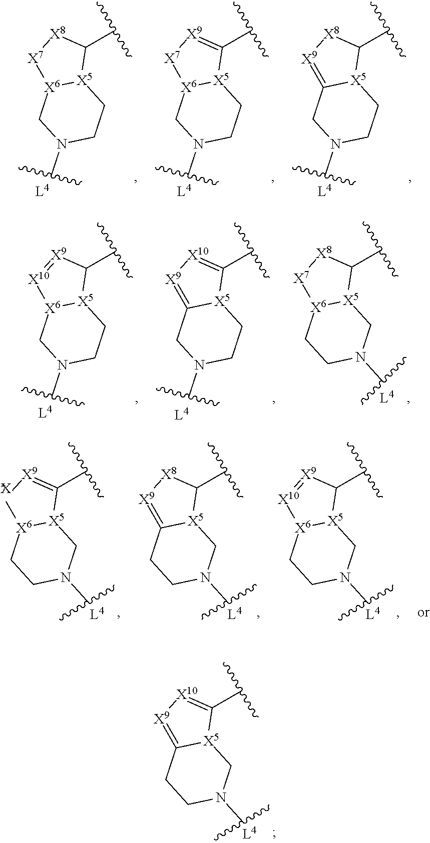

wherein X⁵, X⁶, X⁹, X¹⁰ are independently N or CR¹¹;
X⁷ and X⁸ are independently NH, O, or CHR¹¹;
R¹¹ is hydrogen or C₁₋₆ alkyl;
R⁵ is as defined herein; and

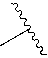

is the point of attachment to the rest of the compound and

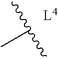

is a bond to L⁴;
wherein if four of any one of X⁵—X¹⁰ are present, at least one is CR¹¹ or CHR¹¹.

In one embodiment of Formula (LP-I), (LP-IA) (LP-IB), (LP-IV), or (LP-IVA), including any of the foregoing, R⁴ is

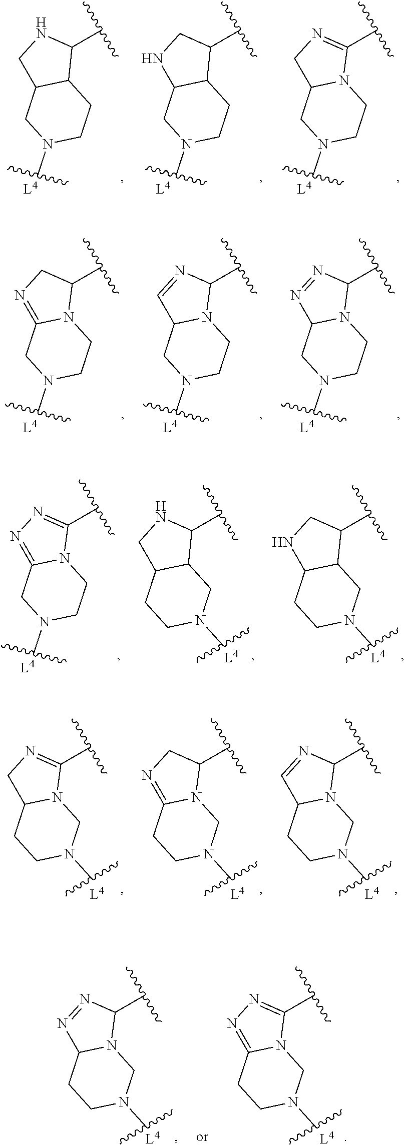

In one embodiment of Formula (LP-J), (LP-IA) (LP-TB), (LP-IV), or (LP-IVA), including any of the foregoing, R⁴ is

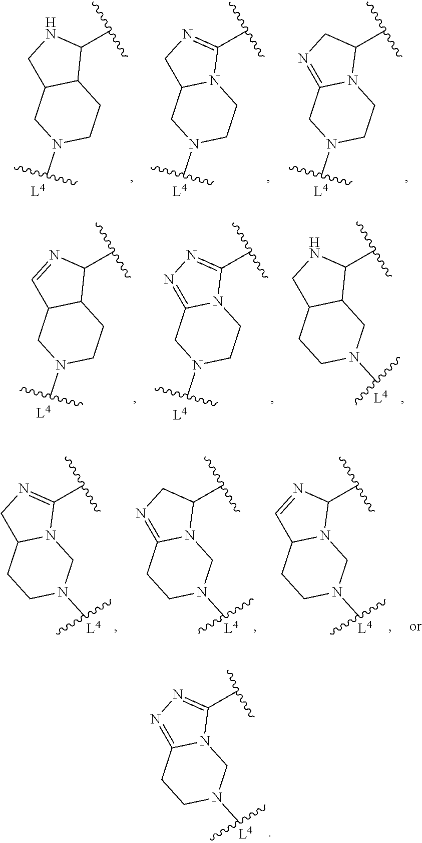

In one embodiment of Formula (LP-I), (LP-IA) (LP-IB), (LP-IV), or (LP-IVA), including any of the foregoing, R⁴ is

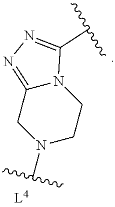 [SMILES: *c1nnc2n1CCN(*)C2]

In one embodiment of Formula (LP-I), (LP-IA) (LP-IB), (LP-IV), or (LP-IVA), including any of the foregoing, Ring A¹ is

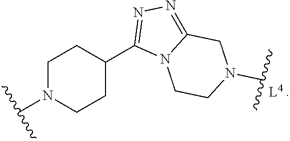 [SMILES: *N1CCC(c2nnc3n2CCN(*)C3)CC1]

In one embodiment of Formula (LP-III) or (LP-IIIA), including any of the foregoing, Ring A³ is an optionally substituted 3- to 12-membered heterocycle optionally substituted with one or more R⁵³. In one embodiment of Formula (LP-III) or (LP-IIIA), including any of the foregoing, Ring A³ is an optionally substituted 8- to 12-membered heterocycle optionally substituted with one or more R⁵³.

In one embodiment of Formula (LP-III) or (LP-IIIA), including any of the foregoing, Ring A³ is a N-linked monocyclic 3- to 12-membered heterocycle comprising a N bound to L² and a N bound to L⁴-RG and wherein the heterocycle is optionally substituted with one or more R⁵³. In one embodiment of Formula (LP-III) or (LP-IIIA), including any of the foregoing, Ring A³ is a N-linked monocyclic 8- to 12-membered heterocycle comprising a N bound to L² and a N bound to L⁴-RG and wherein the heterocycle is optionally substituted with one or more R⁵³.

In one embodiment of Formula (LP-III) or (LP-IIIA), including any of the foregoing, Ring A is

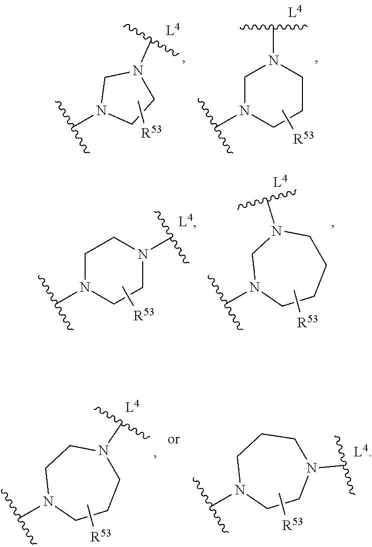

In one embodiment of Formula (LP-III) or (LP-IIIA), including any of the foregoing, Ring A³ is

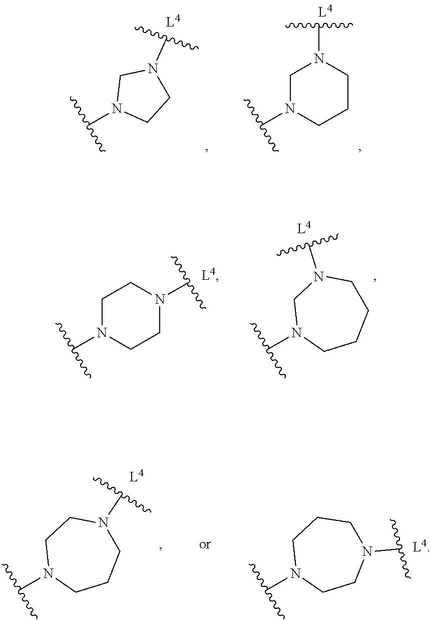 [SMILES: *N1CCN(*)C1 ; *N1CCCN(*)C1 ; *N1CCN(*)CC1 ; *N1CCCCN(*)C1 ; *N1CCCN(*)CC1 ; *N1CCCN(*)CC1]

In one embodiment of Formula (LP-III) or (LP-IIIA), including any of the foregoing, Ring A³ is

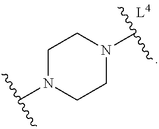 [SMILES: *N1CCN(*)CC1]

In one embodiment of Formula (LP-II), (LP-IIA), (LP-IIB), or (LP-IIC), including any of the foregoing, L⁵ is a linker comprising C₁₋₃ alkyl. In one embodiment of Formula (LP-II), (LP-IIA), (LP-IIB), or (LP-IIC), including any of the foregoing, L⁵ is a linker comprising C₃₋₆ alkyl. In one embodiment of Formula (LP-II), (LP-IIA), (LP-IIB), or (LP-IIC), including any of the foregoing, L⁵ is a linker comprising C₃ alkyl.

In one embodiment of Formula (LP-II), (LP-IIA), (LP-IIB), or (LP-IIC), including any of the foregoing, L⁵ is a linker comprising C₁₋₃ alkyl substituted with R^{18a} and R^{18b} wherein R^{18a} and R^{18b} are joined together with the atoms to which they are attached to form a C₃₋₁₂ carbocycle substituted with -L⁴-RG and further optionally substituted with one or more R⁵². In one embodiment of Formula (LP-II), (LP-IIA), (LP-IIB), or (LP-IIC), including any of the foregoing, L⁵ is a linker comprising C₁₋₃ alkyl substituted with R^{18a} and R^{11b} wherein R^{18a} and R^{18b} are joined together with the atoms to which they are attached to form a C₃₋₆ carbocycle substituted with -L⁴-RG and further optionally substituted with one or more R⁵².

In one embodiment of Formula (LP-II), (LP-IIA), (LP-IIB), or (LP-IIC), including any of the foregoing, L⁵ is a linker comprising C₁₋₃ alkyl substituted with R^{18a} and R^{18b}wherein R^{18a} and R^{18b} are joined together with the atoms to which they are attached to form a 3- to 12-membered heterocycle substituted with -L⁴-RG and further optionally substituted with one or more R⁵². In one embodiment of Formula (LP-II), (LP-IIA), (LP-IIB), or (LP-IIC), including any of the foregoing, L⁵ is a linker comprising C₁₋₃ alkyl substituted with R^{18a} and R^{18b} wherein R^{18a} and R^{18b} are joined together with the atoms to which they are attached to form a 4- to 6-membered heterocycle substituted with -L⁴-RG and further optionally substituted with one or more R⁵². In one embodiment of Formula (LP-II), (LP-IIA), (LP-IIB), or (LP-IIC), including any of the foregoing, L⁵ is a linker comprising C₁₋₃ alkyl substituted with R^{18a} and R^{18b} wherein R^{18a} and R^{18b} are joined together with the atoms to which they are attached to form a 3- to 12-membered heterocycle comprising a N bound to -L⁴-RG and optionally substituted with one or more R⁵². In one embodiment of Formula (LP-II), (LP-IIA), (LP-IIB), or (LP-IIC), including any of the foregoing, L⁵ is a linker comprising C₁₋₃ alkyl substituted with R^{18a} and R^{18b} wherein R^{18a} and R^{18b} are joined together with the atoms to which they are attached to a 3- to 12-membered heterocycle comprising a N bound to -L⁴-RG. In one embodiment of Formula (LP-II), (LP-IIA), (LP-IIB), or (LP-IIC), including any of the foregoing, L⁵ is a linker comprising C₁₋₃ alkyl substituted with R^{18a} and R^{18b}wherein R^{18a} and R^{18b} are joined together with the atoms to which they are attached to a 4- to 6-membered heterocycle comprising a N bound to -L⁴-RG.

In one embodiment of Formula (LP-II), (LP-IIA), (LP-IIB), or (LP-IIC), including any of the foregoing, R^{18a} and R^{18b} are joined together with the atoms to which they are attached to form

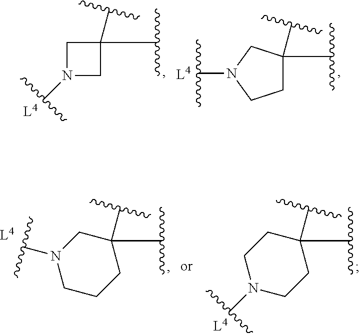 [SMILES: *N1CC(*)(*)C1 ; *N1CCC(*)(*)C1 ; *N1CCCC(*)(*)C1 ; *N1CCC(*)(*)CC1]

wherein

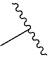

is the point of attachment to the rest of the compound and

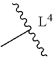

is a bond to L⁴. In one embodiment of Formula (LP-II), (LP-IIA), (LP-IIB), or (LP-IIC), including any of the foregoing, R^{18a} and R^{18b} are joined together with the atoms to which they are attached to form

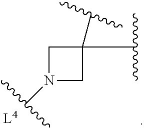 [SMILES: *N1CC(*)(*)C1]

In one embodiment of Formula (LP-V) or (LP-VA), including any of the foregoing, Ring A¹ is selected from

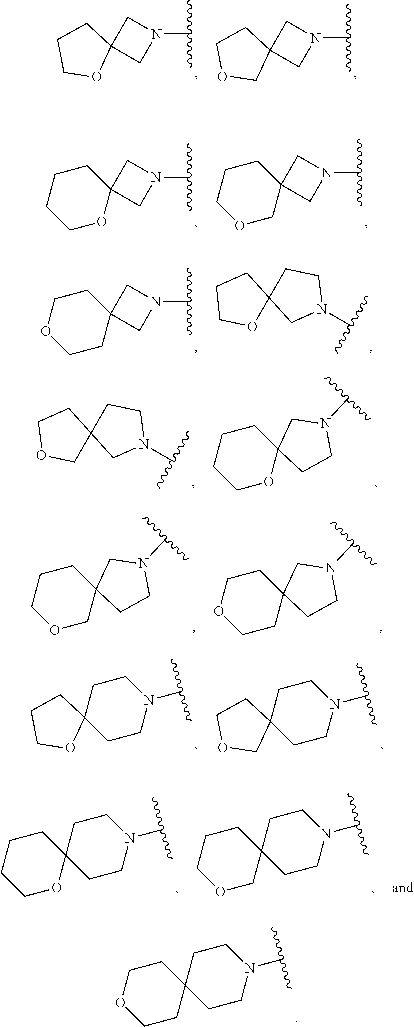

In one embodiment of Formula (LP-V) or (LP-VA), including any of the foregoing, Ring A¹ is

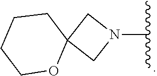 [SMILES: *N1CC2(CCCCO2)C1]

In one embodiment of Formula (LP-V) or (LP-VA), including any of the foregoing, Ring B¹ is an optionally substituted C₃₋₁₂ carbocycle optionally substituted with one or more R⁵². In one embodiment of Formula (LP-V) or (LP-VA), including any of the foregoing, Ring B¹ is an optionally substituted C₆₋₁₂ carbocycle optionally substituted with one or more R⁵². In one embodiment of Formula (LP-V) or (LP-VA), including any of the foregoing, Ring B¹ is an optionally substituted 3- to 12-membered heterocycle optionally substituted with one or more R⁵². In one embodiment of Formula (LP-V) or (LP-VA), including any of the foregoing, Ring B¹ is an optionally substituted 5- to 6-membered heterocycle optionally substituted with one or more R⁵². In one embodiment of Formula (LP-V) or (LP-VA), including any of the foregoing, Ring B¹ is an optionally substituted 3- to 12-membered heterocycle comprising at least a N bound to -L⁴-RG and optionally substituted with one or more R⁵². In one embodiment of Formula (LP-V) or (LP-VA), including any of the foregoing, Ring B¹ is an optionally substituted 4- to 6-membered heterocycle comprising at least a N bound to -L⁴-RG and optionally substituted with one or more R⁵². In one embodiment of Formula (LP-V) or (LP-VA), including any of the foregoing, Ring B¹ is a 3-to 12-membered heterocycle comprising at least a N bound to -L⁴-RG. In one embodiment of Formula (LP-V) or (LP-VA), including any of the foregoing, Ring B¹ is a 4- to 6-membered heterocycle comprising at least a N bound to -L⁴-RG.

In one embodiment of Formula (LP-V) or (LP-VA), including any of the foregoing, Ring B¹ is selected from the group consisting of

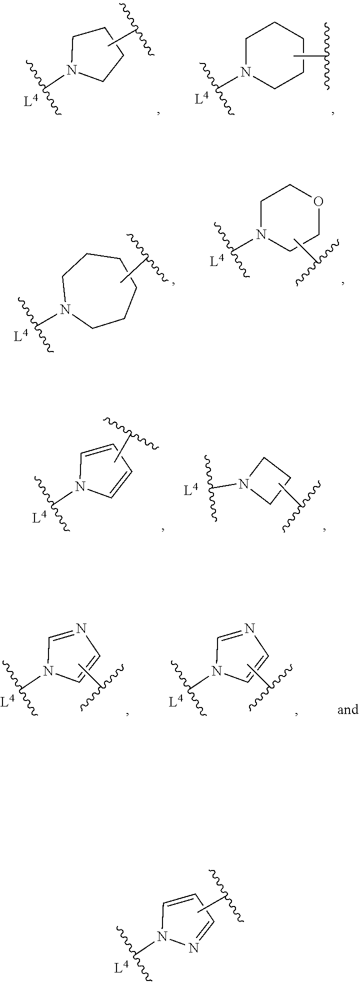

wherein

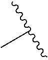

is the point of attachment to the rest of the compound and

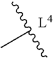

is a bond to L⁴.

In one embodiment of Formula (LP-V) or (LP-VA), including any of the foregoing, Ring B¹ is selected from the group consisting of

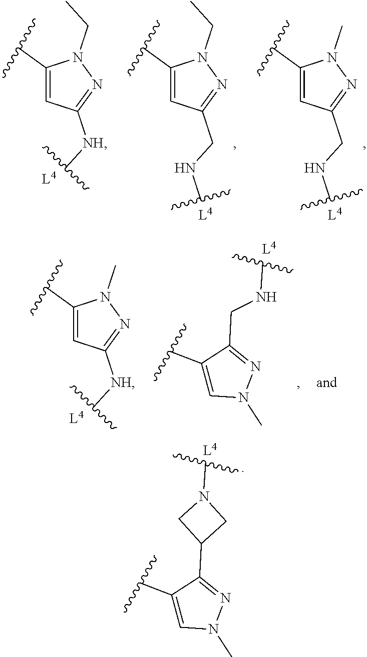

In one embodiment of Formula (LP-V) or (LP-VA), including any of the foregoing, Ring B¹ is

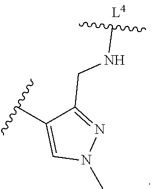 [SMILES: *NCc1nn(C)cc1*]

In one embodiment of Formula (LP-V) or (LP-VA), including any of the foregoing, Ring B¹ is selected from the group consisting of

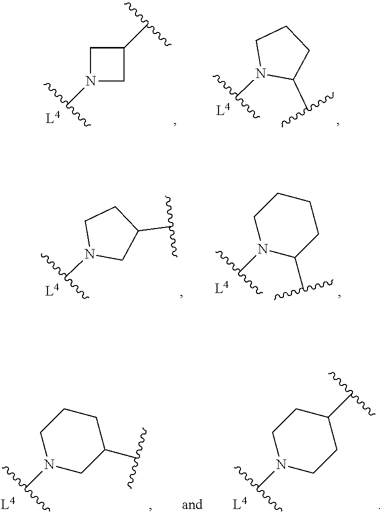 [SMILES: *C1CN(*)C1 ; *C1CCCN1* ; *C1CCN(*)C1 ; *C1CCCCN1* ; *C1CCCN(*)C1 ; *C1CCN(*)CC1]

In one embodiment of Formula (LP-V) or (LP-VA), including any of the foregoing, Ring B¹ is

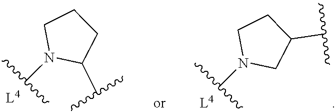 [SMILES: *C1CCCN1* ; *C1CCN(*)C1]

In one embodiment of Formula (LP-V) or (LP-VA), including any of the foregoing, Ring B¹ is

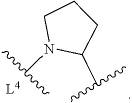 [SMILES: *C1CCCN1*]

In one embodiment of Formula (LP-V) or (LP-VA), including any of the foregoing, Ring B¹ is selected from the group consisting of

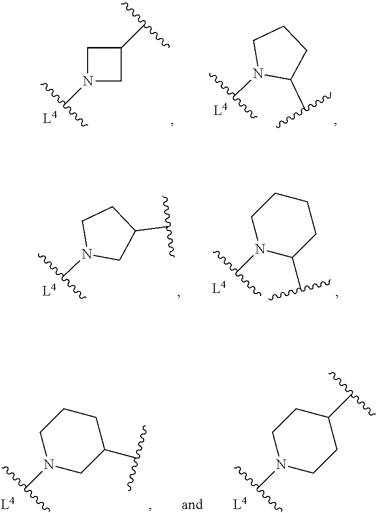 [SMILES: *C1CN(*)C1 ; *C1CCCN1* ; *C1CCN(*)C1 ; *C1CCCCN1* ; *C1CCCN(*)C1 ; *C1CCN(*)CC1]

and Ring A¹ is selected from

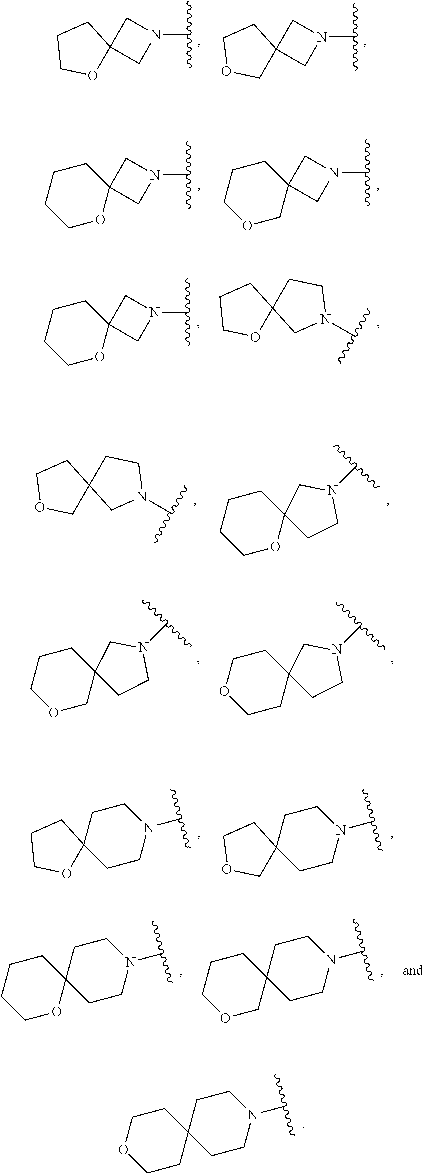

In one embodiment of Formula (LP-V) or (LP-VA), including any of the foregoing, Ring B¹ is

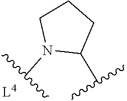 [SMILES: *C1CCCN1*]

### and Ring A¹ is

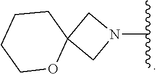 [SMILES: *N1CC2(CCCCO2)C1]

In one embodiment of Formula (LP-V) or (LP-VA), including any of the foregoing, Ring B¹ is

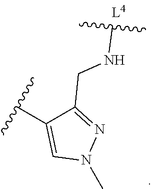 [SMILES: *NCc1nn(C)cc1*]

### and Ring A¹ is

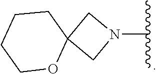 [SMILES: *N1CC2(CCCCO2)C1]

Non-limiting examples of Formula (LP-IA) include:

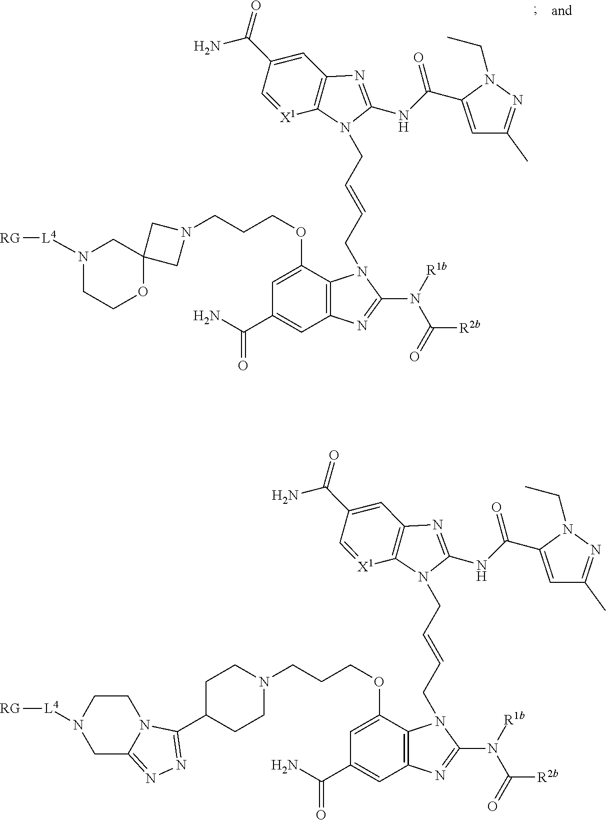

or a stereoisomer thereof.

Non-limiting examples of Formula (LP-IB) include:

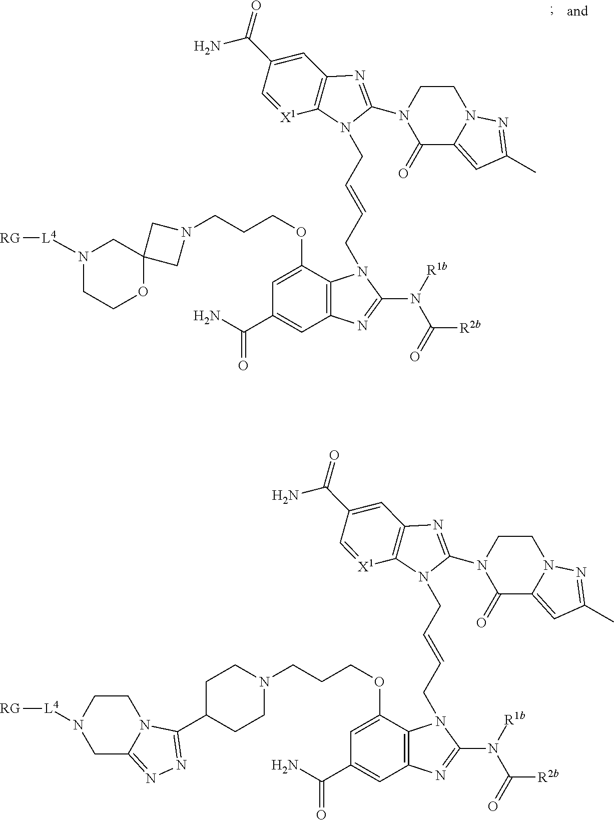

or a stereoisomer thereof.

Non-limiting examples of Formula (LP-IIA), (LP-IIB), or (LP-IIC) include:

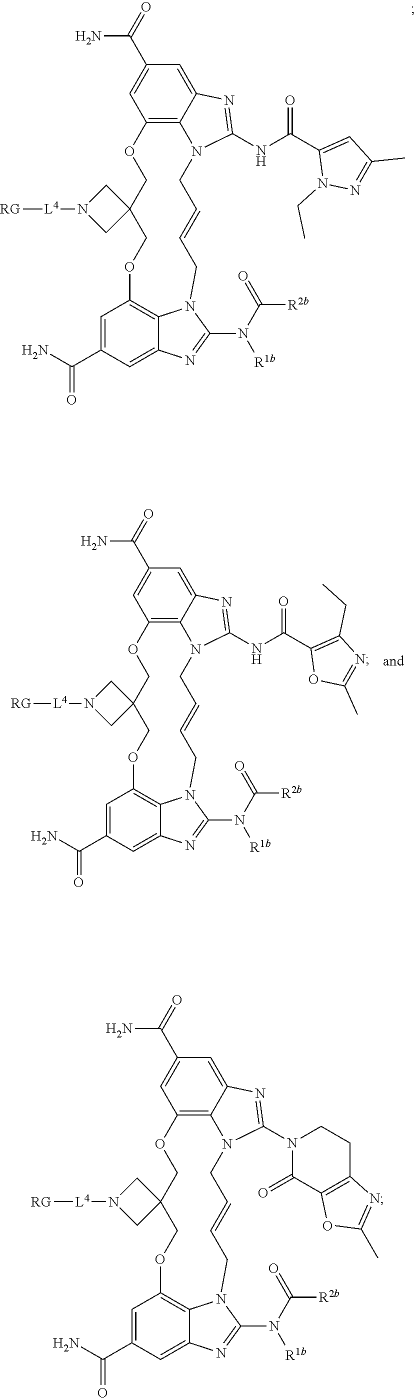

or a stereoisomer thereof.

A non-limiting example of Formula (LP-IIIA) includes:

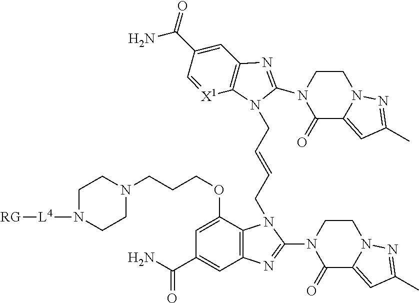

or a stereoisomer thereof.

Non-limiting examples of Formula (LP-IVA) include:

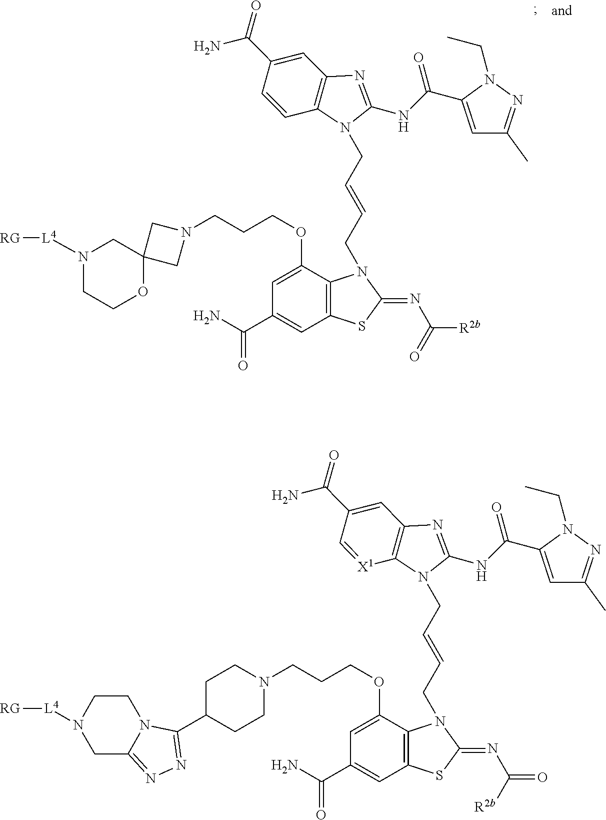

or a stereoisomer thereof.

Non-limiting examples of Formula (LP-VA) include:

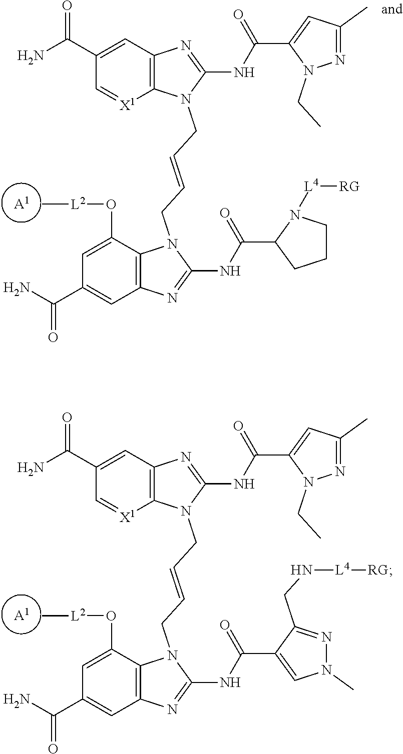

or a stereoisomer thereof.

In one embodiment of Formula (LP-IA), L¹ is —CH═CH—; R^{1a} and R^{1b} are both hydrogen; Ring A¹ is

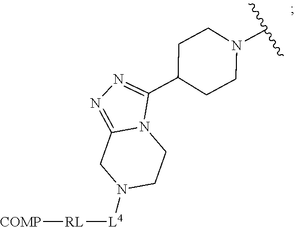

### and R^{2b} is

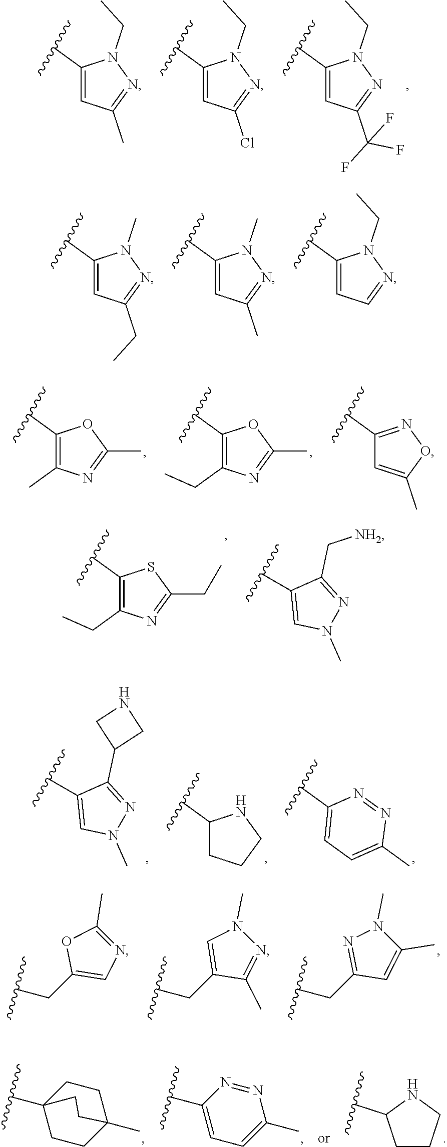

In one embodiment of Formula (LP-IA), L¹ is —CH═CH—; R^{1a} and R^{1b} are both hydrogen; Ring A¹ is

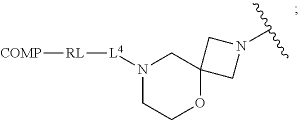

### and R^{2b} is

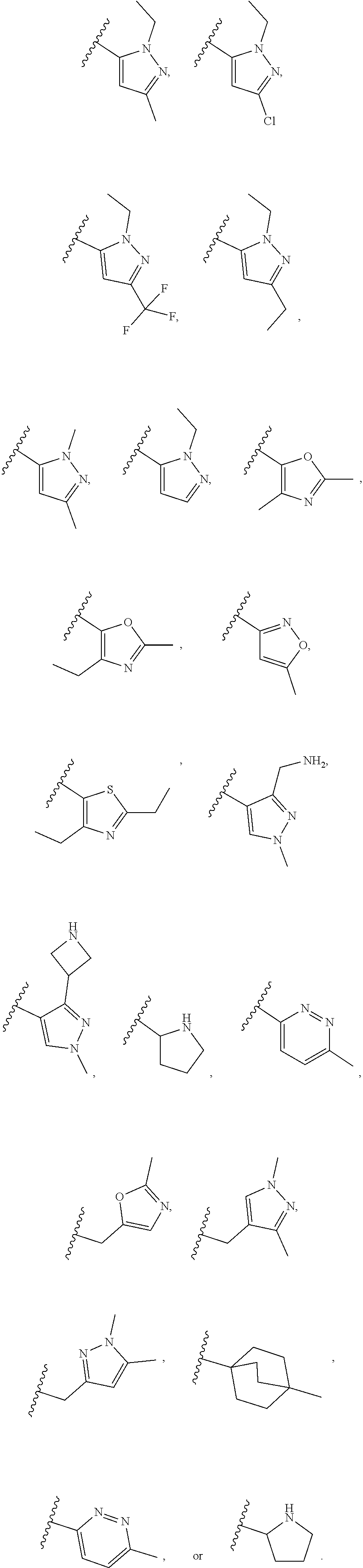

In one embodiment of Formula (LP-IA), L¹ is —CH═CH—; R^{1a} is hydrogen; R^{1b} and R^{2b} are joined together to form

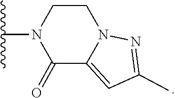 [SMILES: *N1CCn2nc(C)cc2C1=O]

### and Ring A¹ is

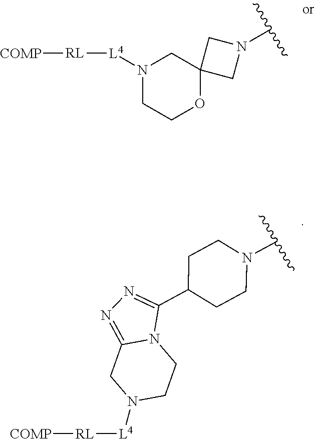

In one embodiment of Formula (LP-IB), L¹ is —CH═CH—; R^{1a} is hydrogen; R^{1b} and R^{2b} are joined together to form

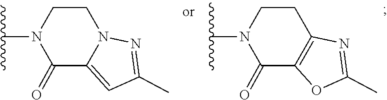 [SMILES: *N1CCn2nc(C)cc2C1=O ; *N1CCc2nc(C)oc2C1=O]

### and Ring A¹ is

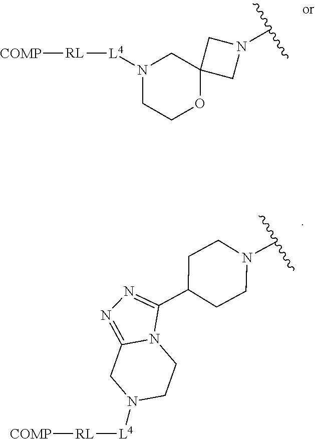

In one embodiment of Formula (LP-IIIA), L¹ is —CH═CH—; R^{9b} and R^{10b} are joined together to form

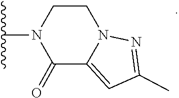 [SMILES: *N1CCn2nc(C)cc2C1=O]

### and Ring A¹ is

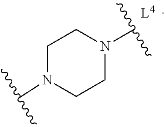 [SMILES: *N1CCN(*)CC1]

In one embodiment of Formula (LP-IVA), L¹ is —CH═CH—; R^{1a} is hydrogen; Ring A¹ is

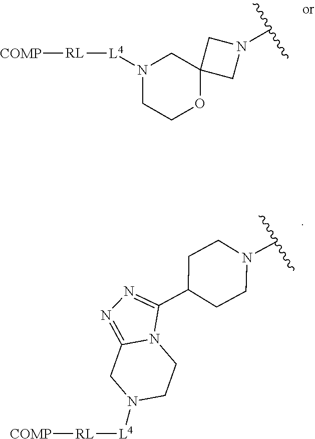

### and R^{2b} is

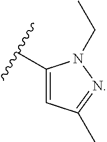 [SMILES: *c1cc(C)nn1CC]

In one embodiment of Formula (LP-VA), L¹ is —CH═CH—; R^{1a} and R^{1b} are both hydrogen; Ring A¹ is

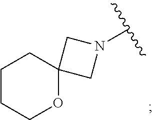 [SMILES: *N1CC2(CCCCO2)C1]

### and Ring B¹ is

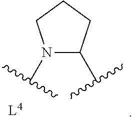

In one embodiment of Formula (LP-VA), L¹ is —CH═CH—; R^{1a} and R^{1b} are both hydrogen; Ring A¹ is

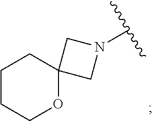 [SMILES: *N1CC2(CCCCO2)C1]

### and Ring B¹ is

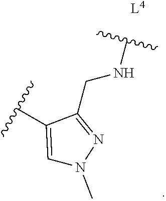

In one embodiment of Formula (LP-IA), L¹ is —CH═CH—; R^{1a} and R^{1b} are both hydrogen; R^{2a} is selected from

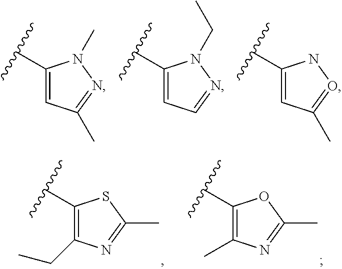

R^{2b} is selected from

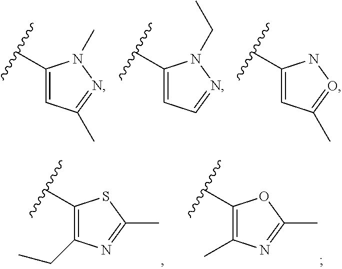

### and Ring A¹ is

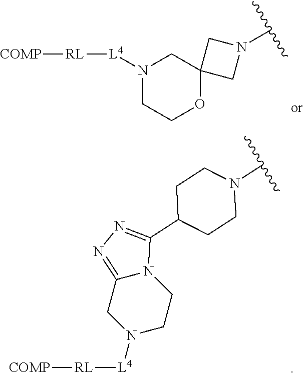

In one embodiment of Formula (LP-IA), L¹ is —CH₂CH₂—; R^{1a} and R^{1b} are both hydrogen; R^{2a} is

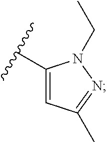 [SMILES: *c1cc(C)nn1CC]

### and R^{2b} is

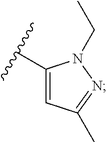 [SMILES: *c1cc(C)nn1CC]

### and Ring A¹ is

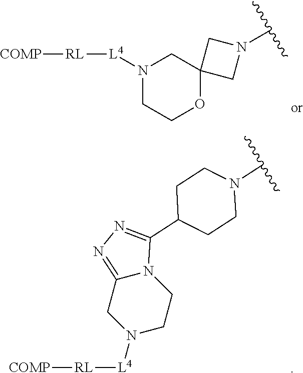

In one embodiment of Formula (LP-I)-(LP-VA), including any of the foregoing, L⁴ is a linker that comprises a protease cleavable linker, a pH-sensitive linker, or a non-cleavable linker.

In one embodiment of Formula (LP-I)-(LP-VA), including any of the foregoing, L⁴ is of the formula:

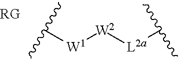

wherein
W¹ and W² are independently absent or a divalent attaching group;
L^{2a} is absent a tease cleavable linker, or a pH-sensitive linker;

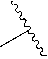

is the point of attachment to the rest of the compound; and

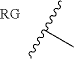

is a bond to RG.

In one embodiment of Formula (LP-I)-(LP-VA), including any of the foregoing, wherein W¹ is C₁₋₆alkylene-, —C(O)—C₁₋₆alkylene-C(O)—, —C(O)(C₁₋₆alkylene)NR¹⁴C(O)—, —C(O)(C₁-6alkylene)OC(O)—, —C(O)(C₁₋₆alkylene)SC(O)—; wherein R¹⁴ is hydrogen or optionally substituted C₁₋₆alkyl, RG is connected to W¹ at —C(O)—, and the C₁₋₆alkylene is optionally substituted with one, two, or three substituents selected from halogen, alkyl, haloalkyl, hydroxyl, amino, alkylamino, and alkoxy.

In one embodiment of Formula (LP-I)-(LP-VA), including any of the foregoing, RG is a group comprising an alkyne, cyclooctyne, a strained alkene, a tetrazine, an amine, methylcyclopropene, a thiol, apara-acetyl-phenylalanine residue, an oxyamine, a maleimide, or an azide. In one embodiment of Formula (LP-I)-(LP-VA), including any of the foregoing, RG comprises an alkyne. In one embodiment of Formula (LP-I)-(LP-VA), including any of the foregoing, RG comprises a cyclooctyne In one embodiment of Formula (LP-I)-(LP-VA), including any of the foregoing, RG comprises a strained alkene. In one embodiment of Formula (LP-I)-(LP-VA), including any of the foregoing, RG comprises a tetrazine. In one embodiment of Formula (LP-I)-(LP-VA), including any of the foregoing, RG comprises an amine. In certain embodiments of Formula (I)—(IH) or (III)—(IIIB), RL comprises a methylcyclopropene. In one embodiment of Formula (LP-I)-(LP-VA), including any of the foregoing, RG comprises a thiol. In one embodiment of Formula (LP-I)-(LP-VA), including any of the foregoing, RG comprises apara-acetyl-phenylalanine residue In one embodiment of Formula (LP-I)-(LP-VA), including any of the foregoing, RG comprises an oxyamine. In one embodiment of Formula (LP-I)-(LP-VA), including any of the foregoing, RG comprises a maleimide. In one embodiment of Formula (LP-I)-(LP-VA), including any of the foregoing, RG comprises an azide. In one embodiment of Formula (LP-I)-(LP-VA), including any of the foregoing, RG comprises is selected from the group consisting of

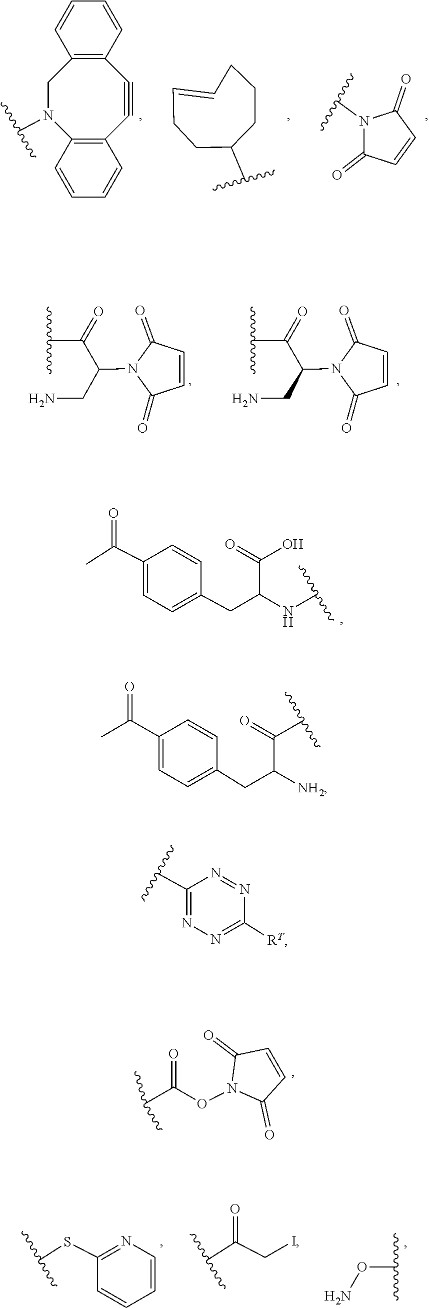

—N₃, —NH₂, methylcyclopropene, and —SH; wherein R^{T} is C₁₋₆ alkyl; and [symbol] represents attachment to the remainder of the compound. In certain embodiments of Formula (I)—(IH) or (III)—(IIIB), RL is

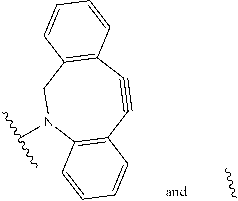

represents attachment to the remainder of the compound In one embodiment of Formula (LP-I)-(LP-VA), including any of the foregoing, RG is

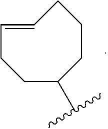 [SMILES: *C1CC/C=C/CCC1]

In one embodiment of Formula (LP-I)-(LP-VA), including any of the foregoing, RG is

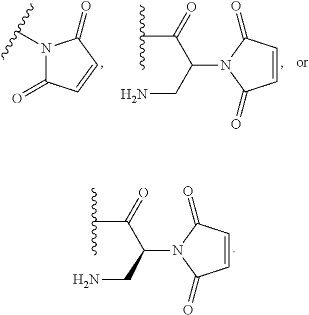 [SMILES: *N1C(=O)C=CC1=O ; *C(=O)C(CN)N1C(=O)C=CC1=O ; *C(=O)[C@H](CN)N1C(=O)C=CC1=O]

In one embodiment of Formula (LP-I)-(LP-VA), including any of the foregoing, RG is

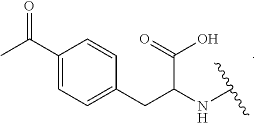 [SMILES: *NC(Cc1ccc(C(C)=O)cc1)C(=O)O]

In one embodiment of Formula (LP-I)-(LP-VA), including any of the foregoing, RG is

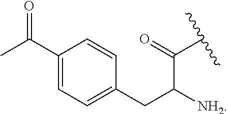 [SMILES: *C(=O)C(N)Cc1ccc(C(C)=O)cc1]

In one embodiment of Formula (LP-I)-(LP-VA), including any of the foregoing, RG is

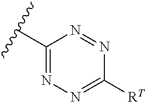

wherein R^{T} is C₁₋₆ alkyl. In certain embodiments, R^{T} is methyl, ethyl, or propyl. In certain embodiments, R^{T} is methyl. In certain embodiments, R^{T} is ethyl. In certain embodiments, R^{T} is propyl. In certain embodiments, R^{T} is butyl. In certain embodiments, R^{T} is pentyl. In certain embodiments, R^{T} is hexyl In one embodiment of Formula (LP-I)-(LP-VA), including any of the foregoing, RG is

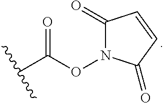 [SMILES: *C(=O)ON1C(=O)C=CC1=O]

In one embodiment of Formula (LP-I)-(LP-VA), including any of the foregoing, RG is

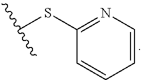 [SMILES: *Sc1ccccn1]

In one embodiment of Formula (LP-I)-(LP-VA), including any of the foregoing, RG is

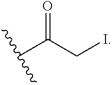 [SMILES: *C(=O)CI]

In one embodiment of Formula (LP-I)-(LP-VA), including any of the foregoing, RG is

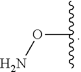 [SMILES: *ON]

In one embodiment of Formula (LP-I)-(LP-VA), including any of the foregoing, RG is —N₃. In one embodiment of Formula (LP-I)-(LP-VA), including any of the foregoing, RG is —NH₂. In one embodiment of Formula (LP-I)-(LP-VA), including any of the foregoing, RG is methylcyclopropene. In one embodiment of Formula (LP-I)-(LP-VA), including any of the foregoing, RG is —SH.

In one embodiment, the compound of (LP-I)-(LP-V) is a linker payload of Table B or Table B-1 or a pharmaceutically acceptable salt or tautomer thereof:

TABLE B

| Compd | |
|---|---|
| No. | Structure |
| LP- 1-1 | 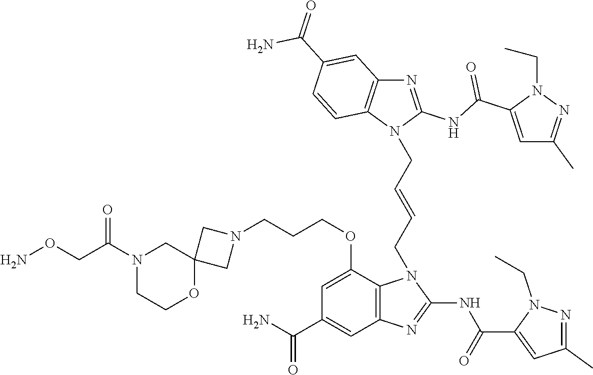 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)CON)CCO4)c21] |
| LP- 1-2 | 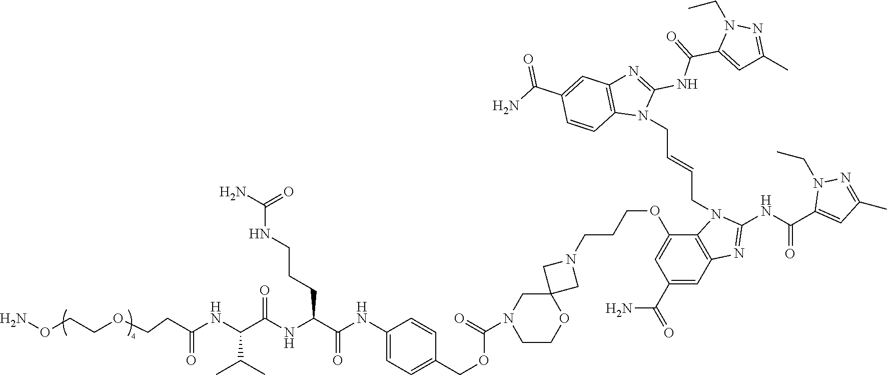 |
| LP- 1-3 | 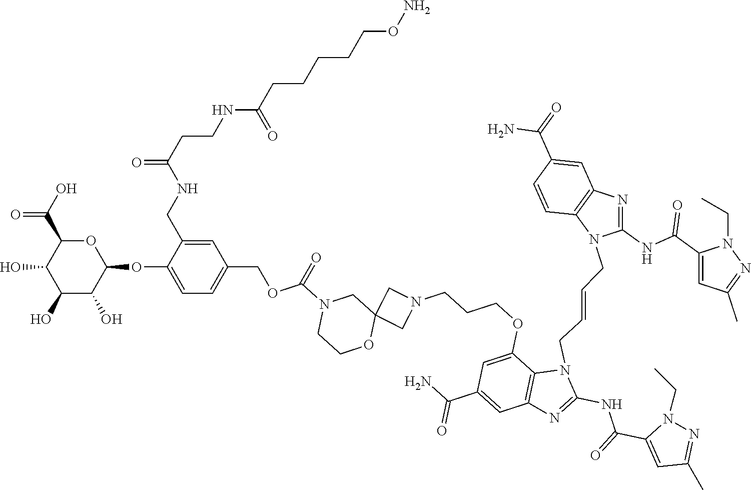 |
| LP- 1-4 | 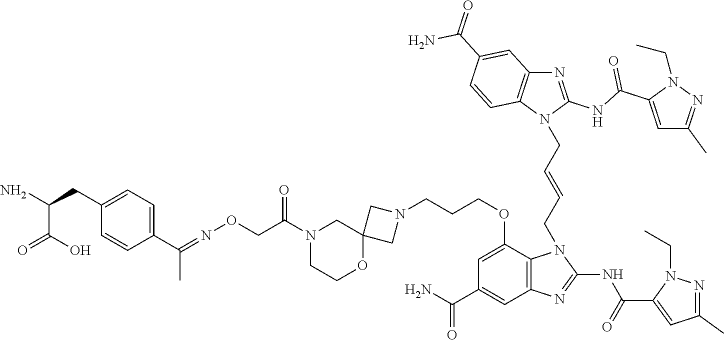 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)CON=C(C)c3ccc(C[C@H](N)C(=O)O)cc3)CCO4)c21] |
| LP- 2-1 | 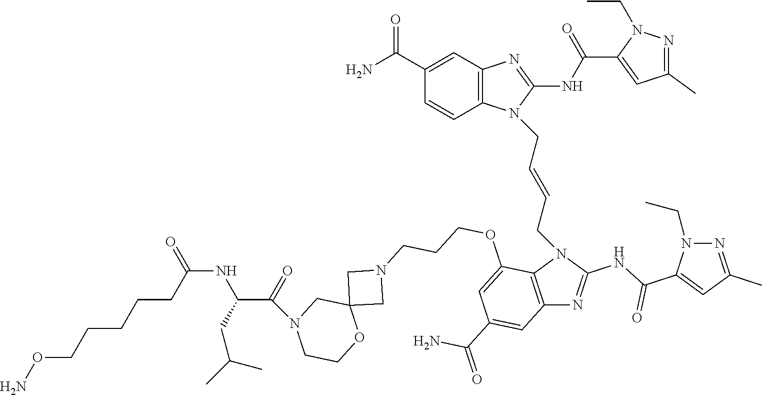 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@H](CC(C)C)NC(=O)CCCCCON)CCO4)c21] |
| LP- 2-2 | 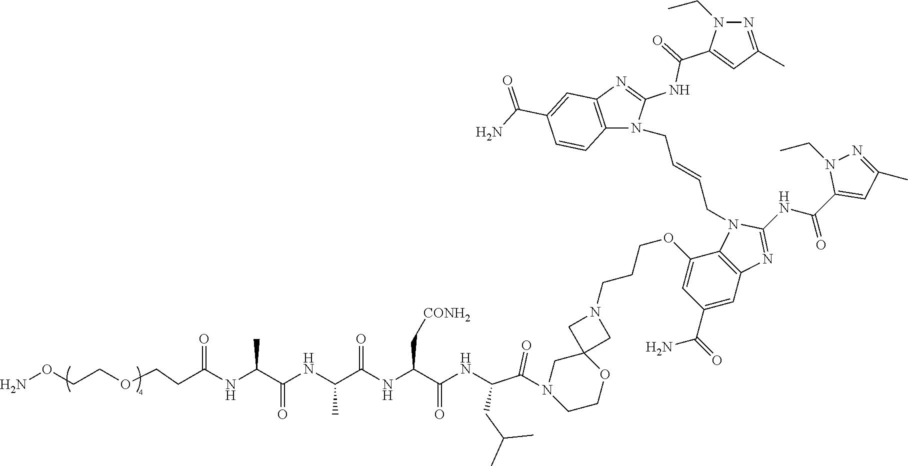 |
| LP- 2-3 | 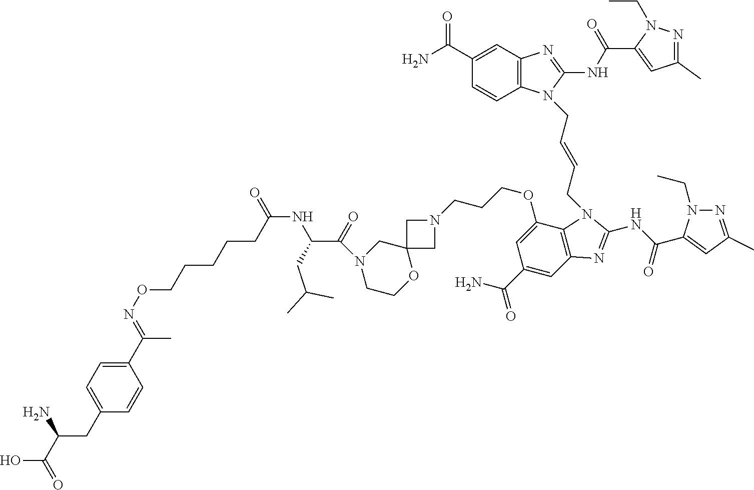 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@H](CC(C)C)NC(=O)CCCCCO/N=C(\C)c3ccc(C[C@H](N)C(=O)O)cc3)CCO4)c21] |
| LP- 2-4 | 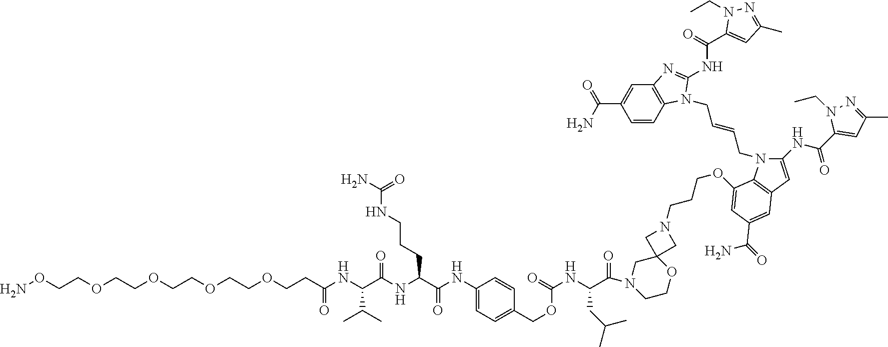 [SMILES: CCn1nc(C)cc1C(=O)Nc1nc2cc(C(N)=O)ccc2n1C/C=C/Cn1c(NC(=O)c2cc(C)nn2CC)cc2cc(C(N)=O)cc(OCCCN3CC4(C3)CN(C(=O)[C@H](CC(C)C)NC(=O)OCc3ccc(NC(=O)[C@H](CCCNC(N)=O)NC(=O)[C@@H](NC(=O)CCOCCOCCOCCOCCON)C(C)C)cc3)CCO4)c21] |
| LP- 4-1 | 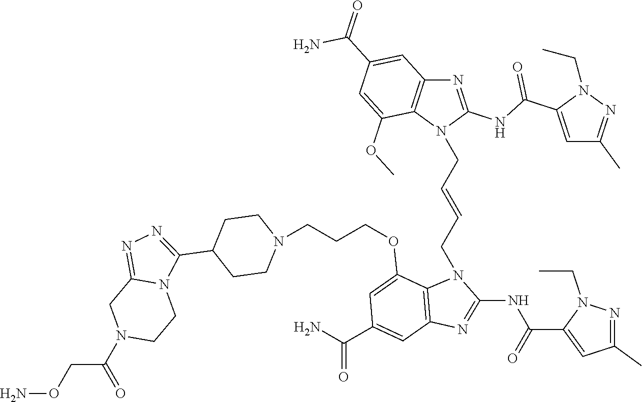 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21] |
| LP- 4-2 | 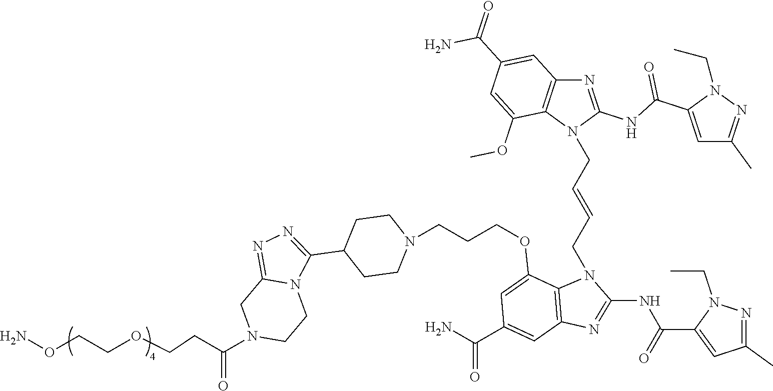 |
| LP- 4-3 | 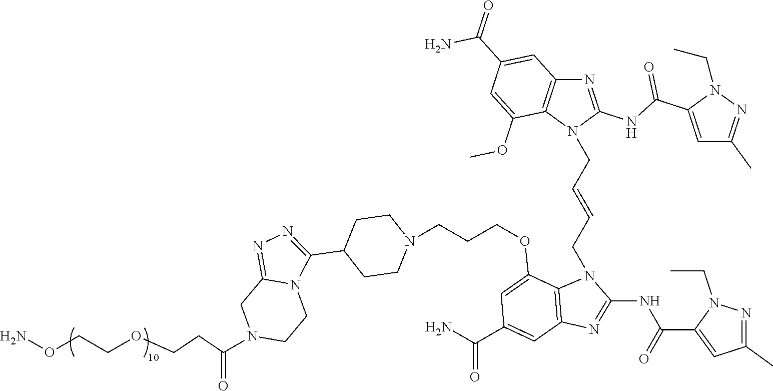 |
| LP- 4-4 | 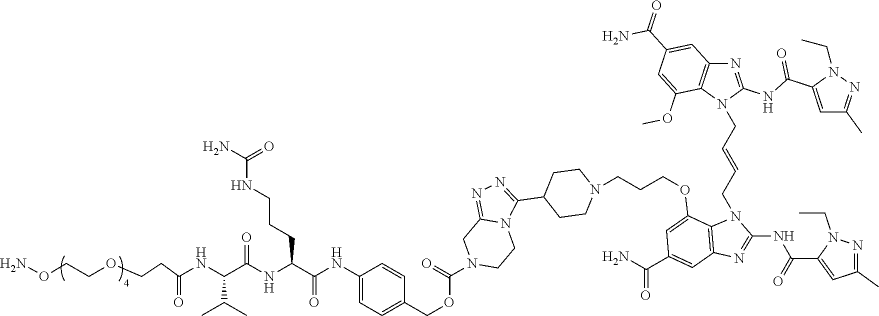 |
| LP- 4-5 | 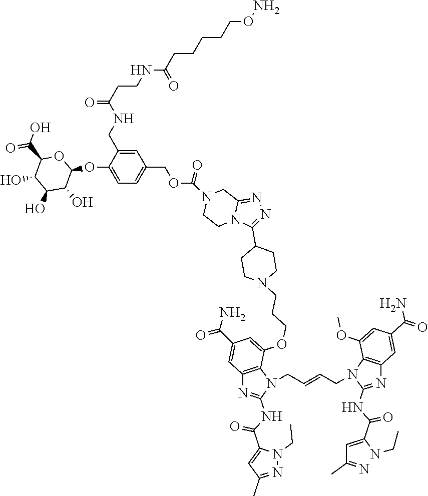 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(O[C@@H]6O[C@H](C(=O)O)[C@@H](O)[C@H](O)[C@H]6O)c(CNC(=O)CCNC(=O)CCCCCON)c4)C5)CC3)c21] |
| LP- 4-6 | 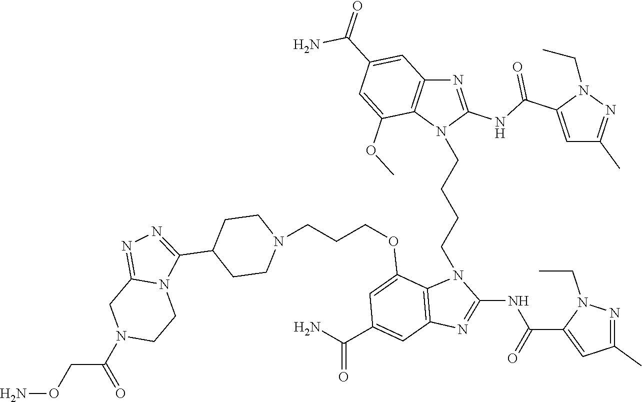 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCN(C(=O)CON)C5)CC3)c21] |
| LP- 4-7 | 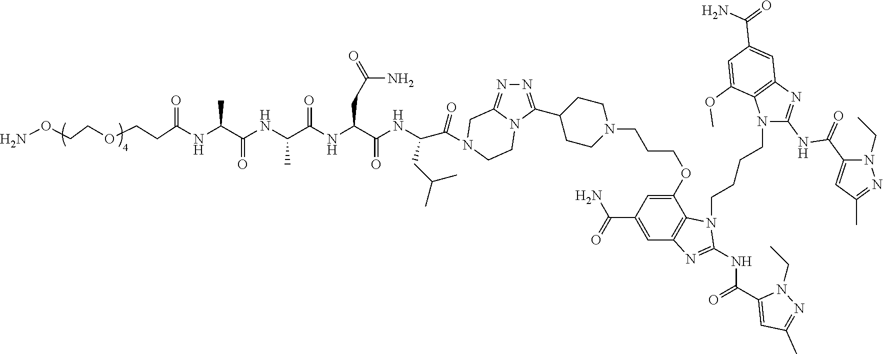 |
| LP- 4-8 | 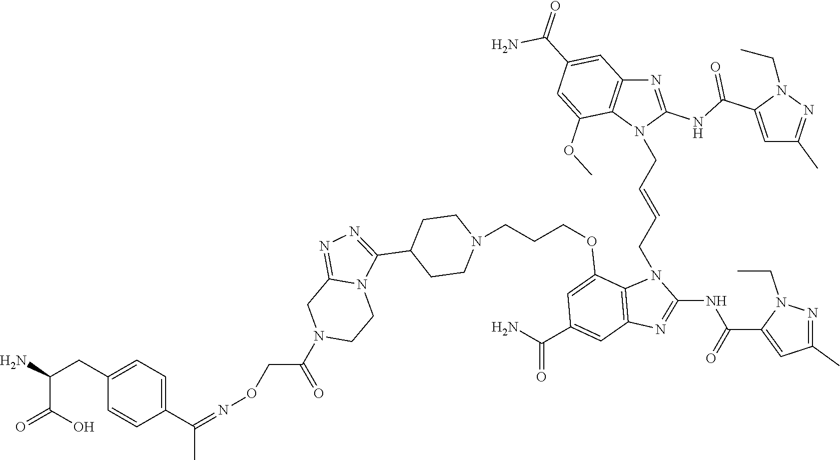 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(C[C@H](N)C(=O)O)cc4)C5)CC3)c21] |
| LP- 4-9 | 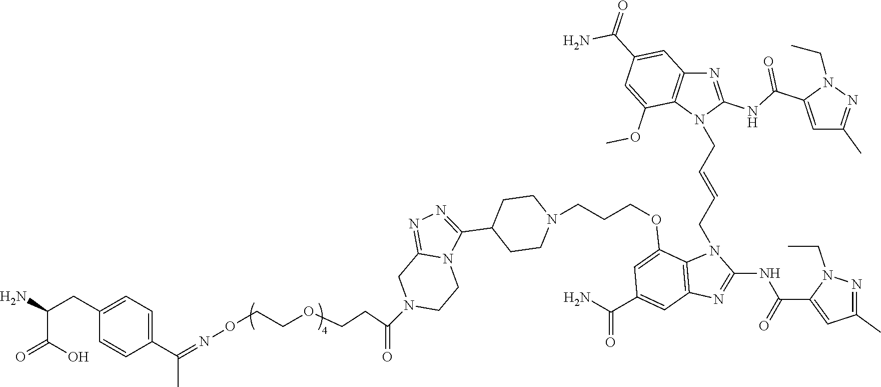 |
| LP- 4-10 | 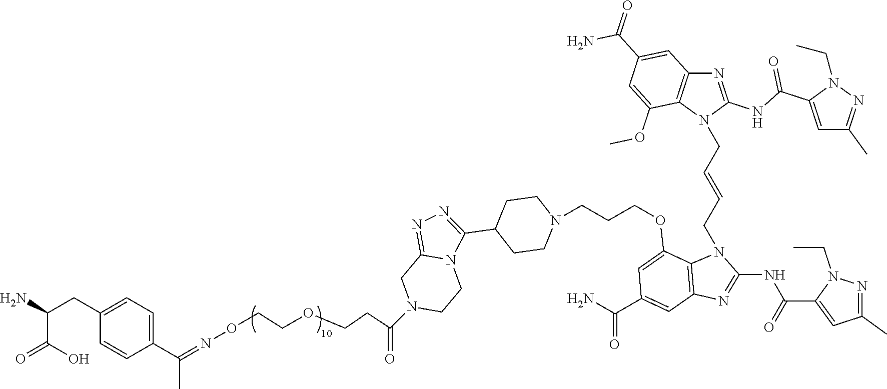 |
| LP- 4-11 | 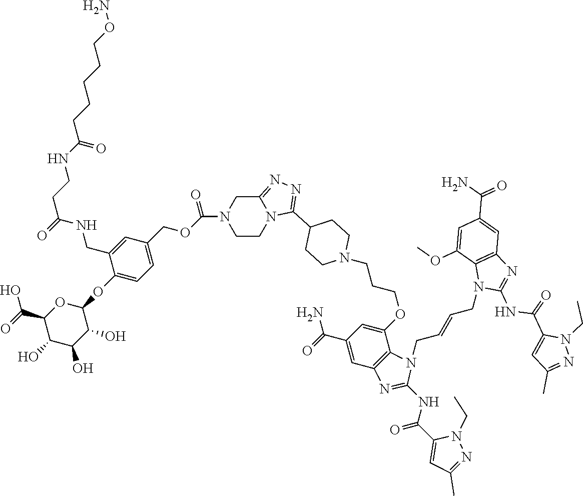 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(O[C@@H]6O[C@H](C(=O)O)[C@@H](O)[C@H](O)[C@H]6O)c(CNC(=O)CCNC(=O)CCCCCON)c4)C5)CC3)c21] |
| LP- 4-12 | 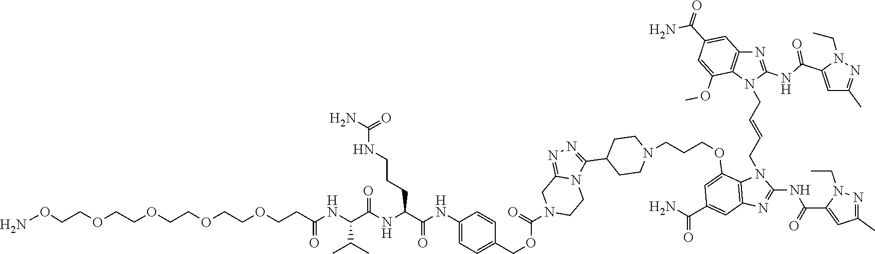 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(NC(=O)[C@H](CCCNC(N)=O)NC(=O)[C@@H](NC(=O)CCOCCOCCOCCOCCON)C(C)C)cc4)C5)CC3)c21] |
| LP- 4-14 | 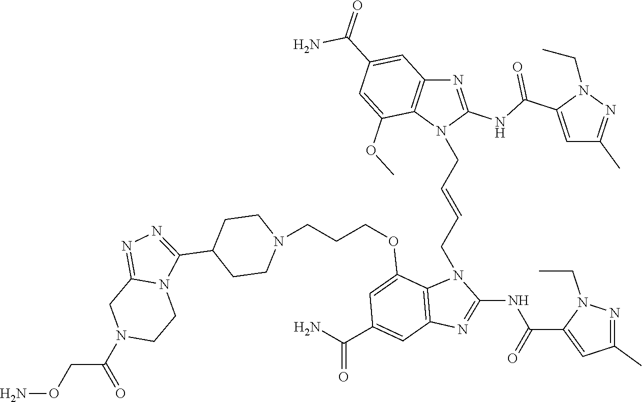 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21] |
| LP- 4-15 | 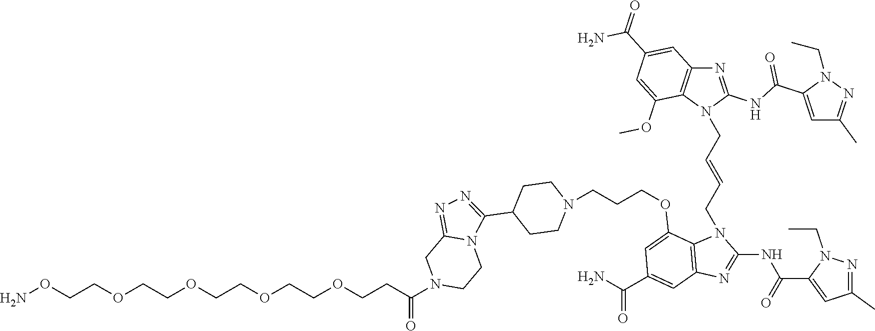 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCON)C5)CC3)c21] |
| LP- 4-16 | 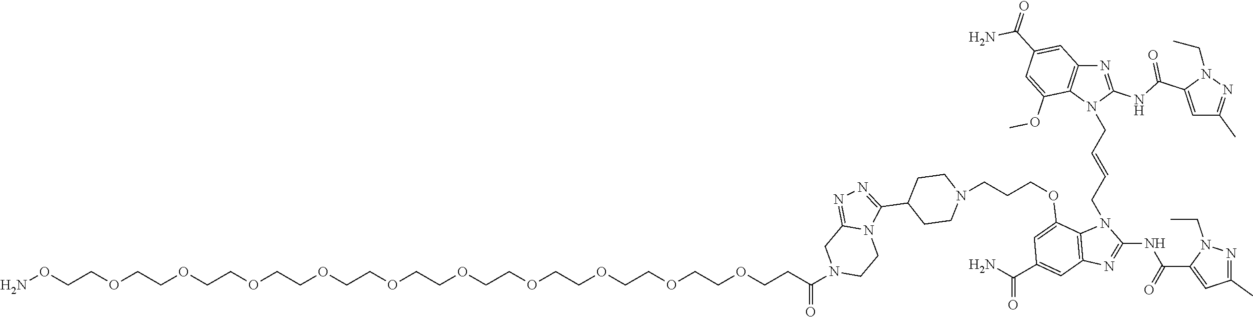 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCOCCOCCOCCOCCOCCOCCON)C5)CC3)c21] |
| LP- 4-17 | 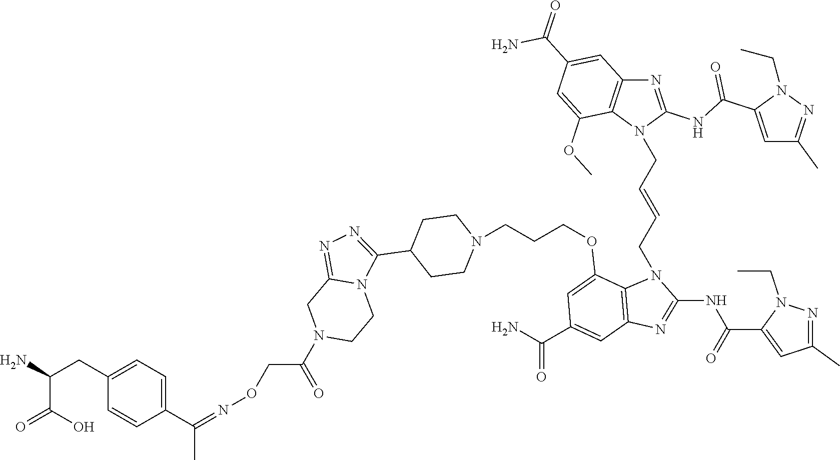 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(C[C@H](N)C(=O)O)cc4)C5)CC3)c21] |
| LP- 4-18 | 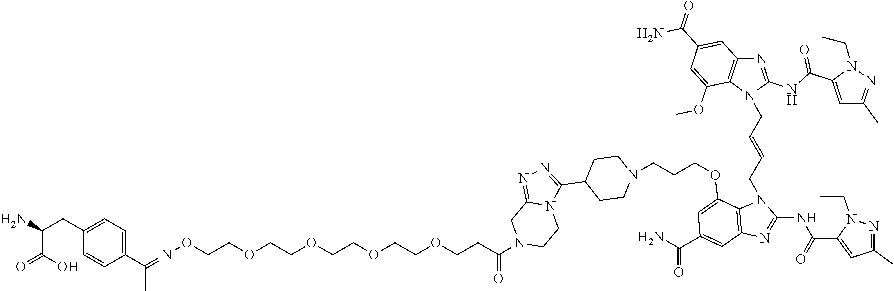 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCON=C(C)c4ccc(C[C@H](N)C(=O)O)cc4)C5)CC3)c21] |
| LP- 4-19 | 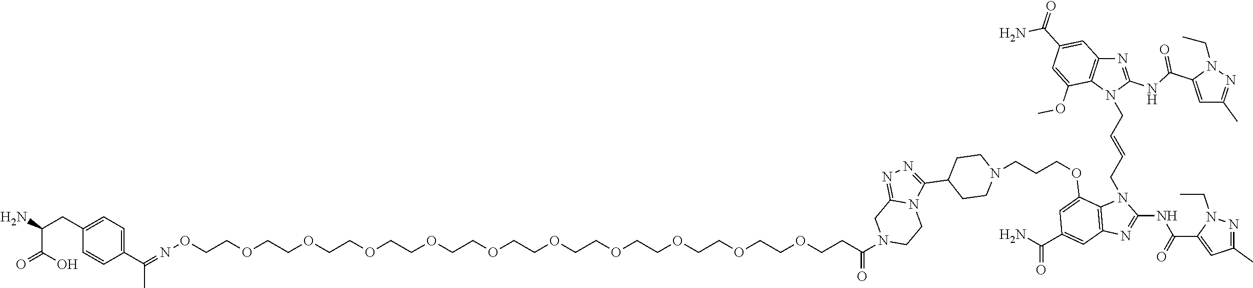 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCOCCOCCOCCOCCOCCOCCON=C(C)c4ccc(C[C@H](N)C(=O)O)cc4)C5)CC3)c21] |
| LP- 4-20 | 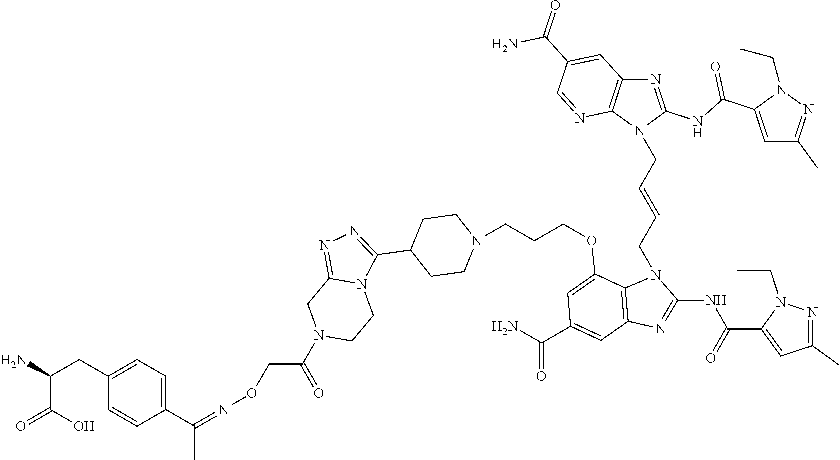 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(C[C@H](N)C(=O)O)cc4)C5)CC3)c21] |
| LP- 5-1 | 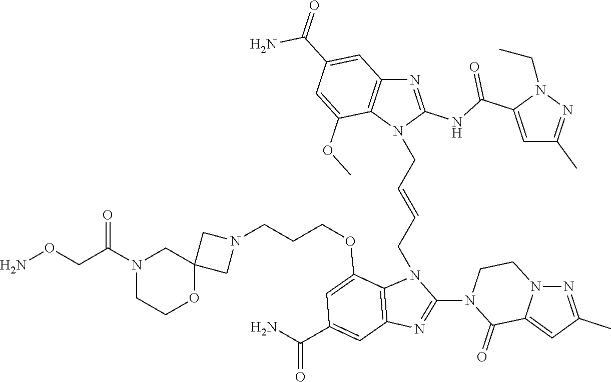 [SMILES: CCn1nc(C)cc1C(=O)Nc1nc2cc(C(N)=O)cc(OC)c2n1C/C=C/Cn1c(N2CCn3nc(C)cc3C2=O)nc2cc(C(N)=O)cc(OCCCN3CC4(C3)CN(C(=O)CON)CCO4)c21] |
| LP- 6-1 | 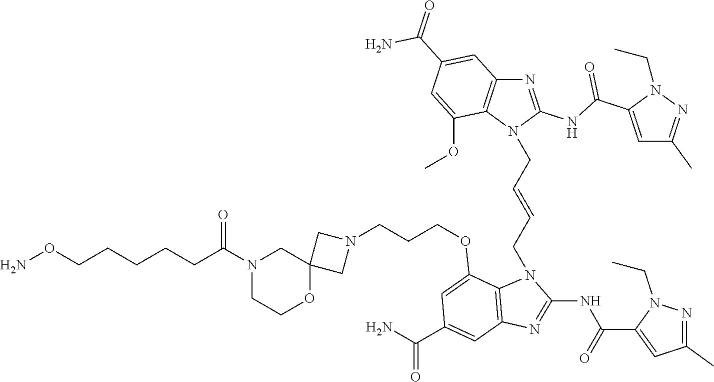 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(C3)CN(C(=O)CCCCCON)CCO4)c21] |
| LP- 10-1 | 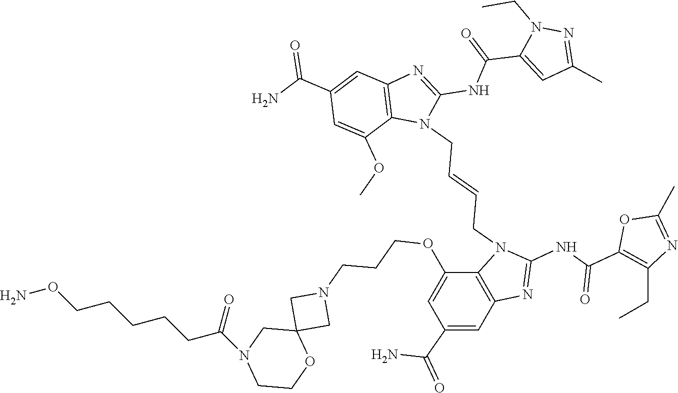 [SMILES: CCc1nc(C)oc1C(=O)Nc1nc2cc(C(N)=O)cc(OCCCN3CC4(C3)CN(C(=O)CCCCCON)CCO4)c2n1C/C=C/Cn1c(NC(=O)c2cc(C)nn2CC)nc2cc(C(N)=O)cc(OC)c21] |
| LP- 12-1 | 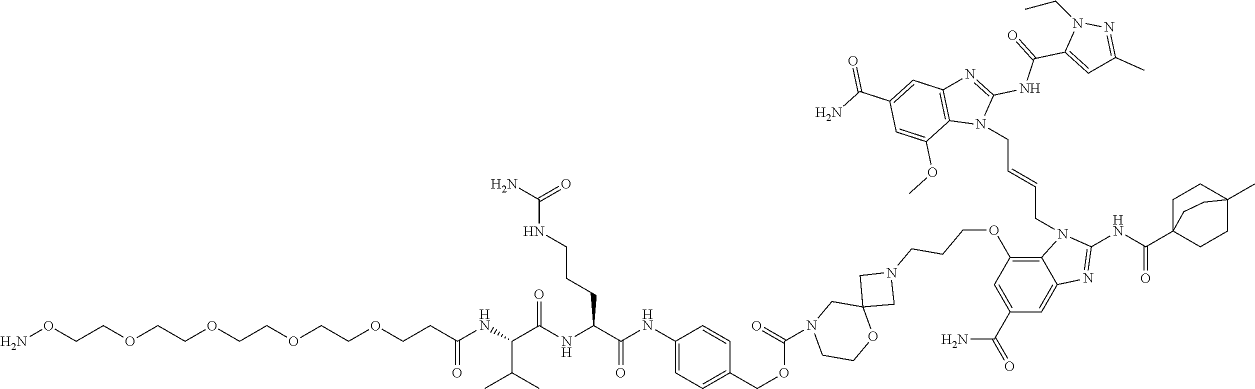 [SMILES: CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(C3)CN(C(=O)OCc3ccc(NC(=O)[C@H](CCCNC(N)=O)NC(=O)[C@@H](NC(=O)CCOCCOCCOCCOCCON)C(C)C)cc3)CCO4)c21] |
| LP- 12-2 | 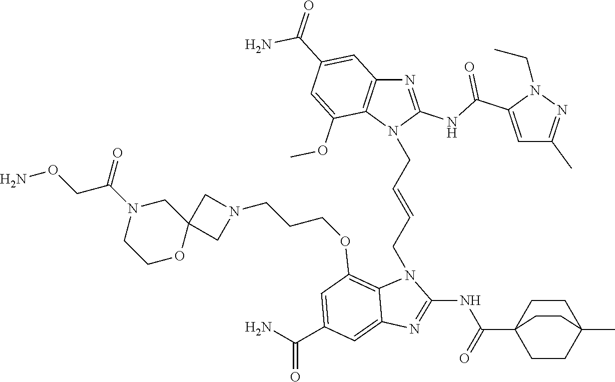 [SMILES: CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(C3)CN(C(=O)CON)CCO4)c21] |
| LP- 16-1 | 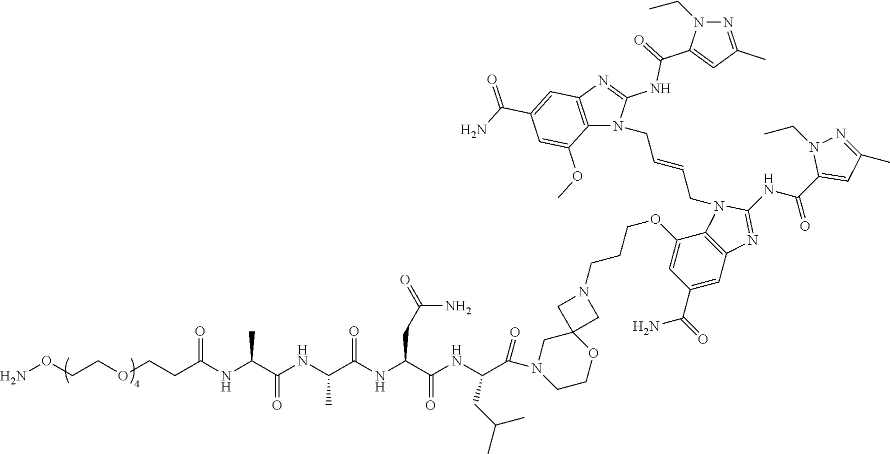 |
| LP- 16-2 | 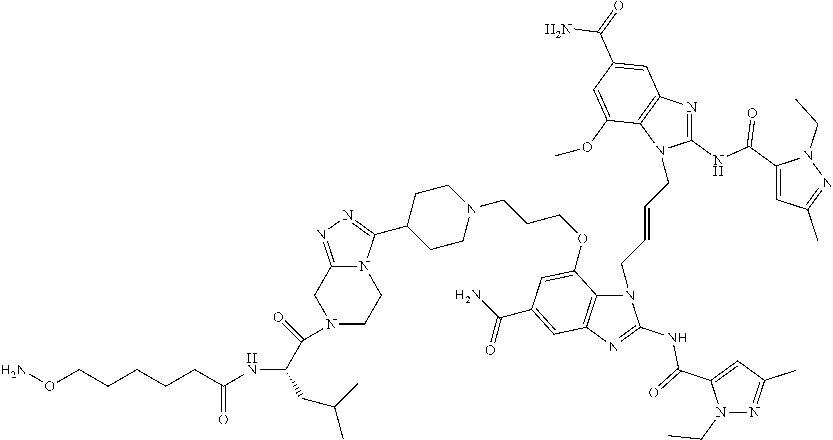 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)CCCCCON)C5)CC3)c21] |
| LP- 21-1 | 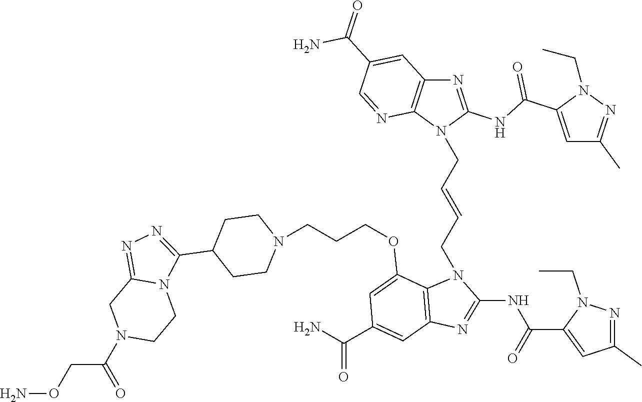 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CON)C5)CC3)c21] |
| LP- 21-2 | 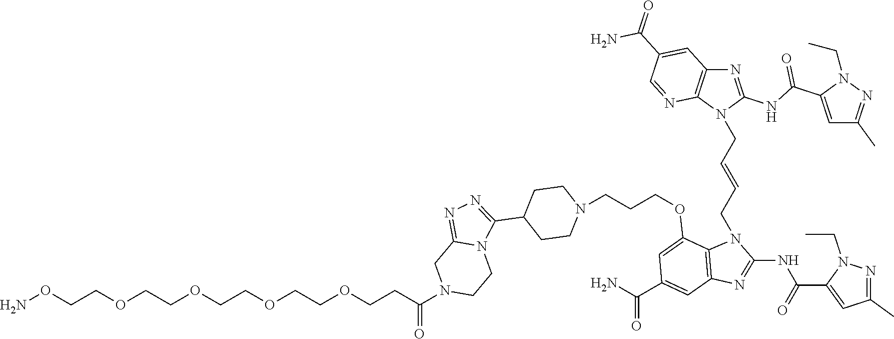 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCON)C5)CC3)c21] |
| LP- 21-3 | 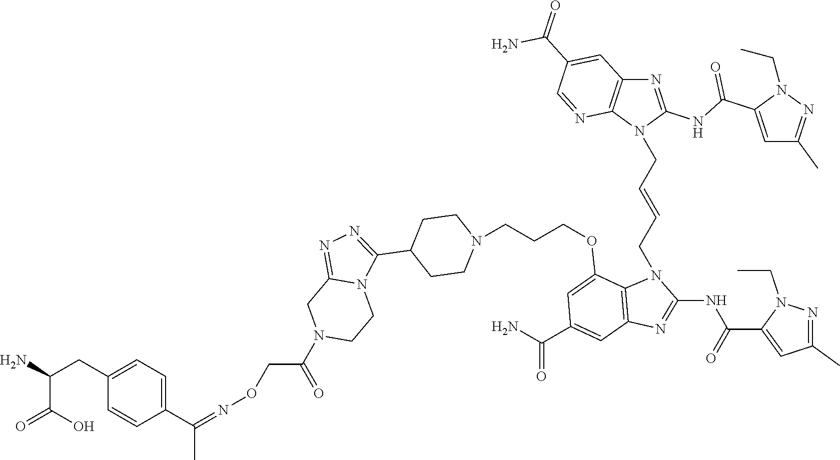 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(C[C@H](N)C(=O)O)cc4)C5)CC3)c21] |
| LP- 25-1 | 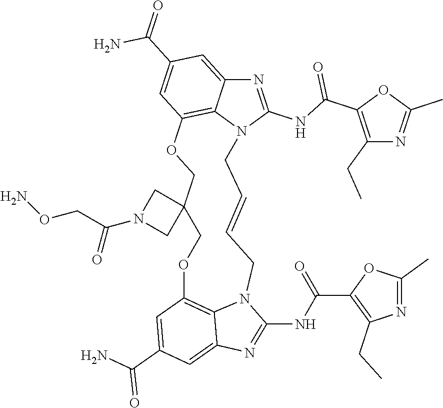 [SMILES: CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CO3)CN(C(=O)CON)C1] |
| LP- 25-2 | 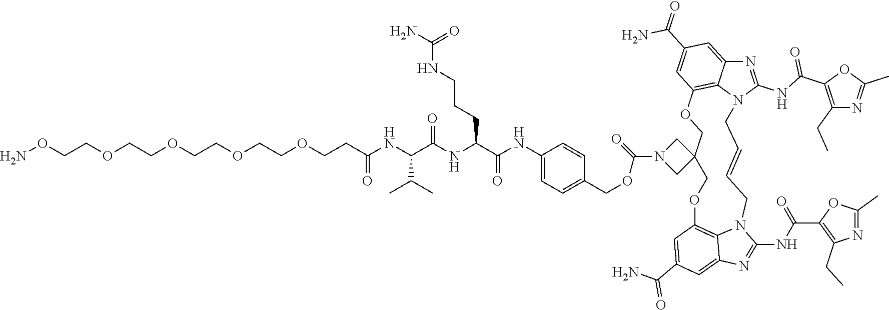 [SMILES: CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CO3)CN(C(=O)OCc2ccc(NC(=O)[C@H](CCCNC(N)=O)NC(=O)[C@@H](NC(=O)CCOCCOCCOCCOCCON)C(C)C)cc2)C1] |
| LP- 49-1 | 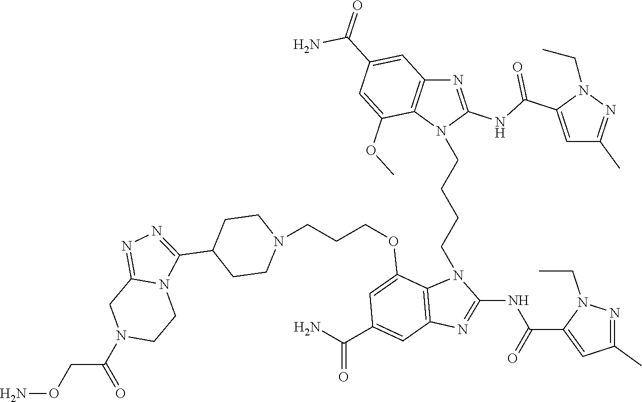 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCN(C(=O)CON)C5)CC3)c21] |
| LP- 49-2 | 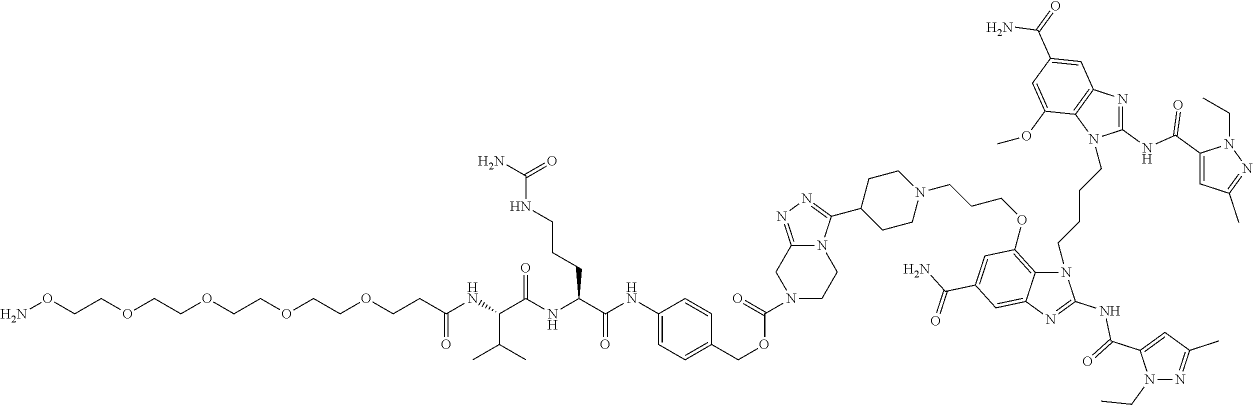 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCN(C(=O)OCc4ccc(NC(=O)[C@H](CCCNC(N)=O)NC(=O)[C@@H](NC(=O)CCOCCOCCOCCOCCON)C(C)C)cc4)C5)CC3)c21] |
| LP- 57-1 | 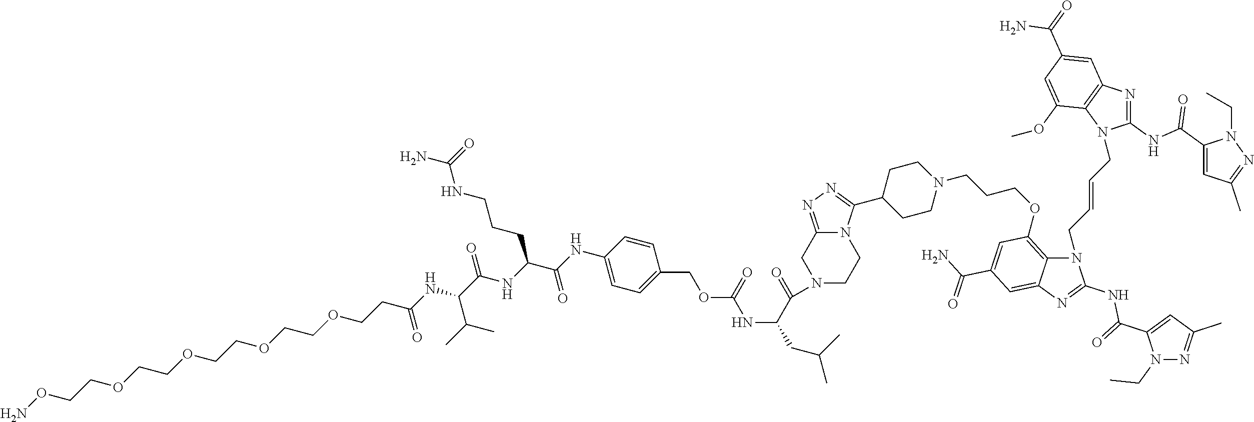 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)OCc4ccc(NC(=O)[C@H](CCCNC(N)=O)NC(=O)[C@@H](NC(=O)CCOCCOCCOCCOCCON)C(C)C)cc4)C5)CC3)c21] |
| LP- 57-2 | 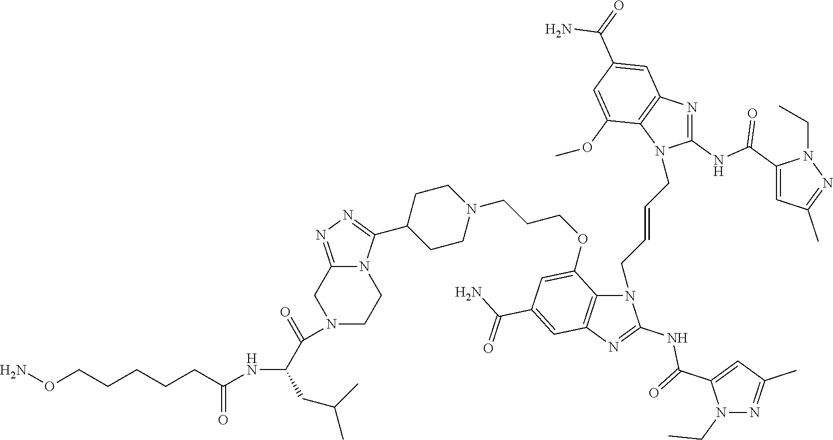 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)CCCCCON)C5)CC3)c21] |
| LP- 57-3 | 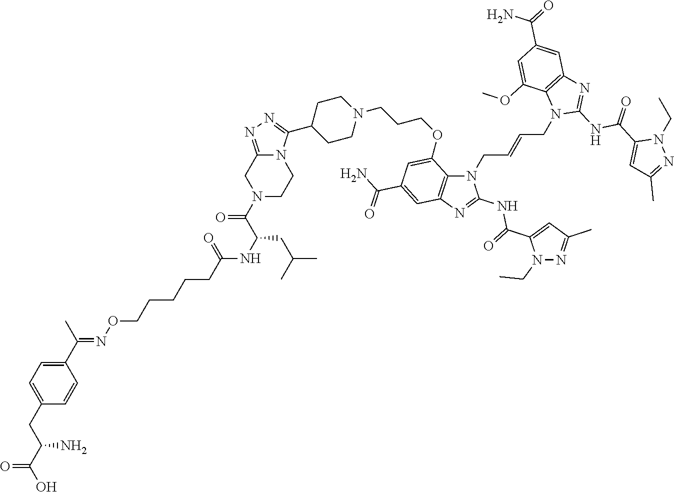 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)CCCCCO/N=C(\C)c4ccc(C[C@H](N)C(=O)O)cc4)C5)CC3)c21] |
| LP- 58-1 | 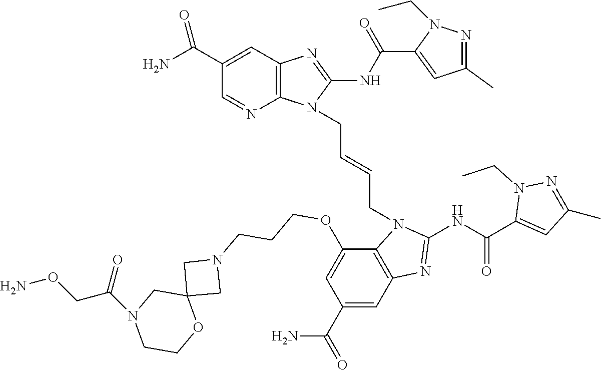 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CC4(C3)CN(C(=O)CON)CCO4)c21] |
| LP- 59-1 | 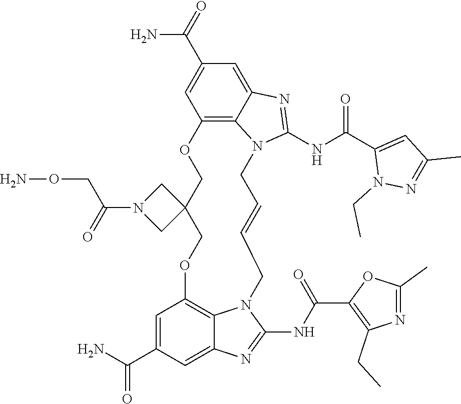 [SMILES: CCc1nc(C)oc1C(=O)Nc1nc2cc(C(N)=O)cc3c2n1C/C=C/Cn1c(NC(=O)c2cc(C)nn2CC)nc2cc(C(N)=O)cc(c21)OCC1(CO3)CN(C(=O)CON)C1] |
| LP- 60-1 | 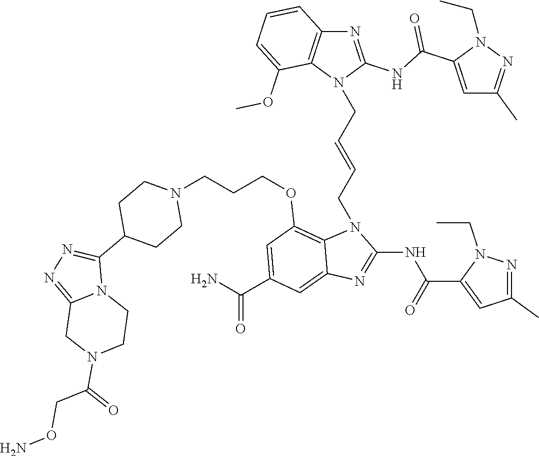 [SMILES: CCn1nc(C)cc1C(=O)Nc1nc2cccc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21] |
| LP- 63-1 | 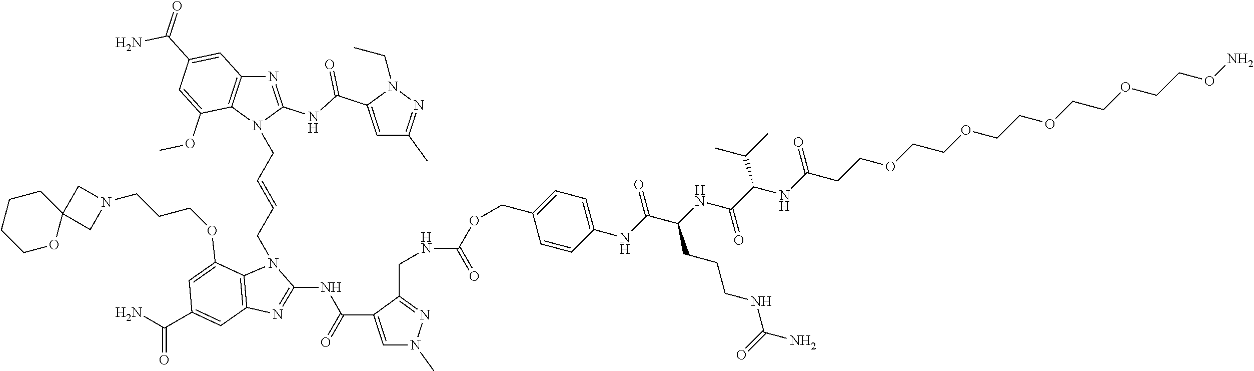 [SMILES: CCn1nc(C)cc1C(=O)Nc1nc2cc(C(N)=O)cc(OC)c2n1C/C=C/Cn1c(NC(=O)c2cn(C)nc2CNC(=O)OCc2ccc(NC(=O)[C@H](CCCNC(N)=O)NC(=O)[C@@H](NC(=O)CCOCCOCCOCCOCCON)C(C)C)cc2)nc2cc(C(N)=O)cc(OCCCN3CC4(CCCCO4)C3)c21] |
| LP- 65-1 | 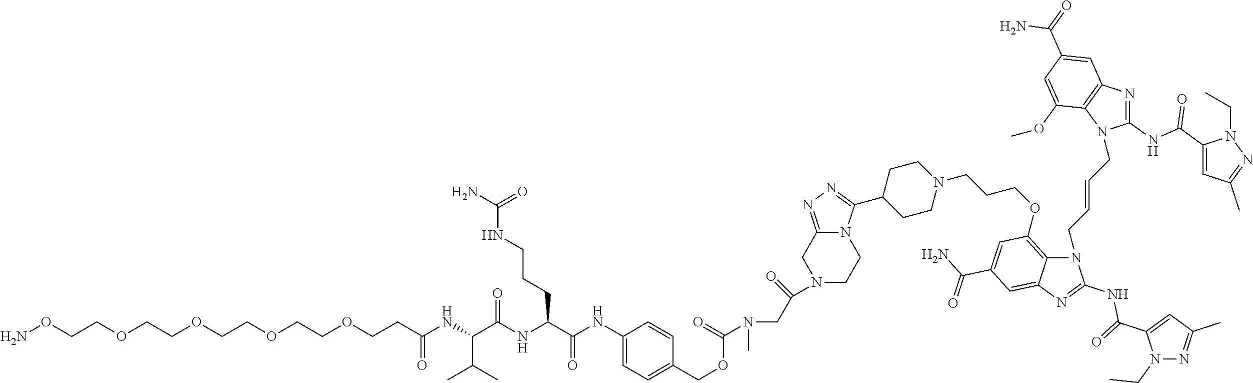 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CN(C)C(=O)OCc4ccc(NC(=O)[C@H](CCCNC(N)=O)NC(=O)[C@@H](NC(=O)CCOCCOCCOCCOCCON)C(C)C)cc4)C5)CC3)c21] |
| LP- 65-2 | 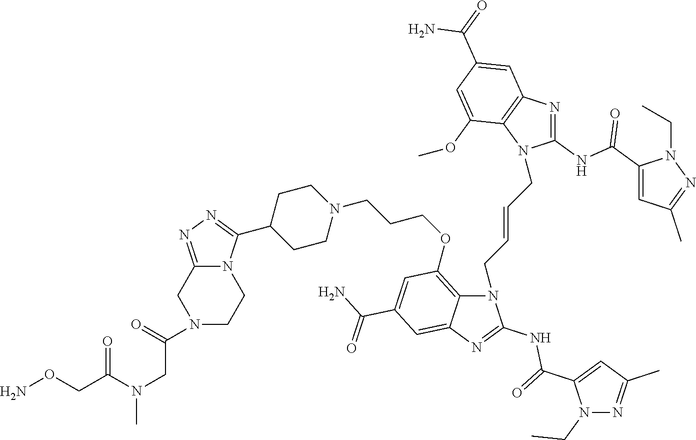 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CN(C)C(=O)CON)C5)CC3)c21] |
| LP- 66-1 | 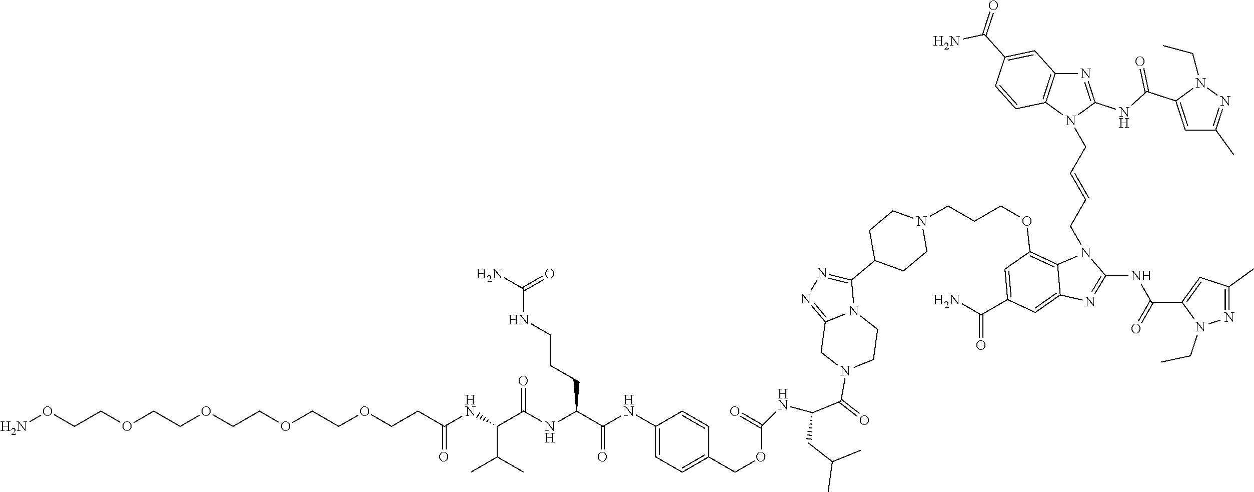 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)OCc4ccc(NC(=O)[C@H](CCCNC(N)=O)NC(=O)[C@@H](NC(=O)CCOCCOCCOCCOCCON)C(C)C)cc4)C5)CC3)c21] |

TABLE B-1

| | |
|---|---|
| LP- B1 | 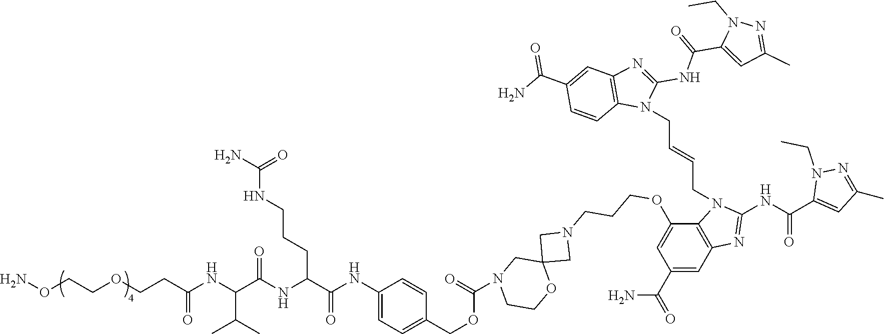 |
| LP- B2 | 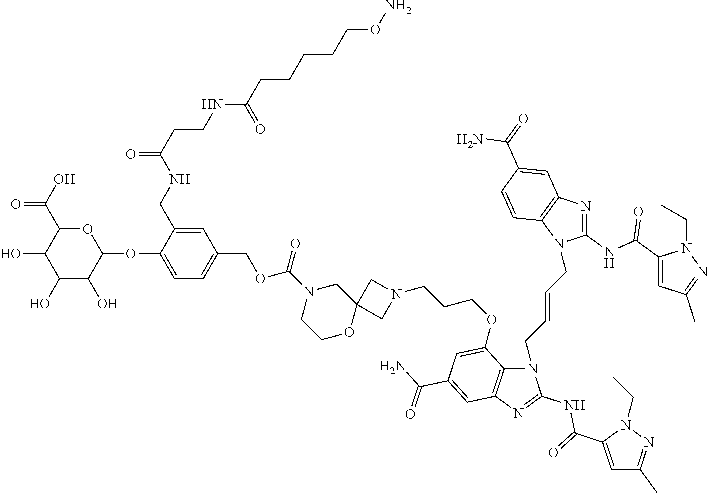 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)OCc3ccc(OC5OC(C(=O)O)C(O)C(O)C5O)c(CNC(=O)CCNC(=O)CCCCCON)c3)CCO4)c21] |
| LP- B3 | 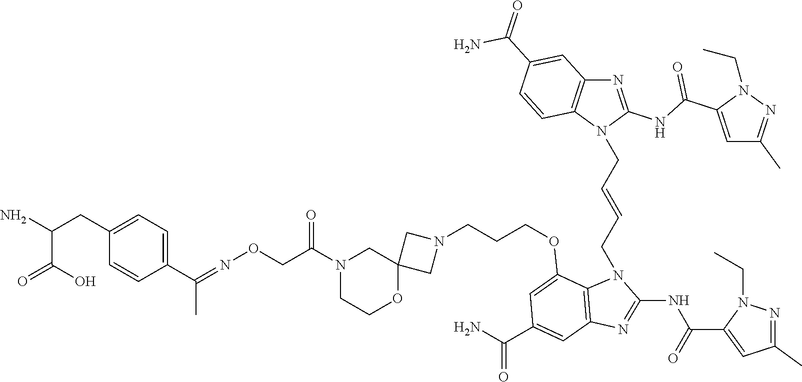 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)CON=C(C)c3ccc(CC(N)C(=O)O)cc3)CCO4)c21] |
| LP- B4 | 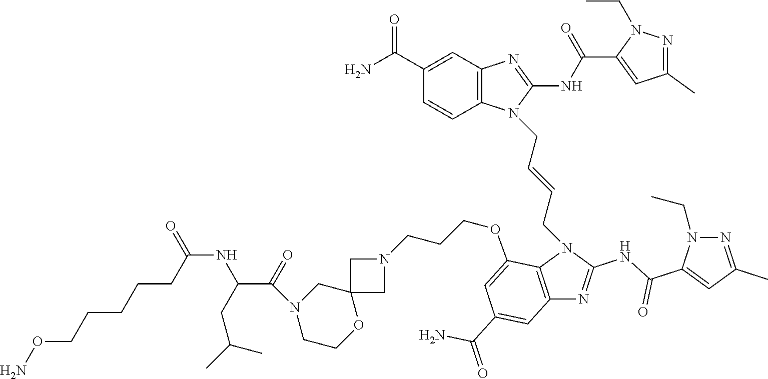 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)C(CC(C)C)NC(=O)CCCCCON)CCO4)c21] |
| LP- B5 | 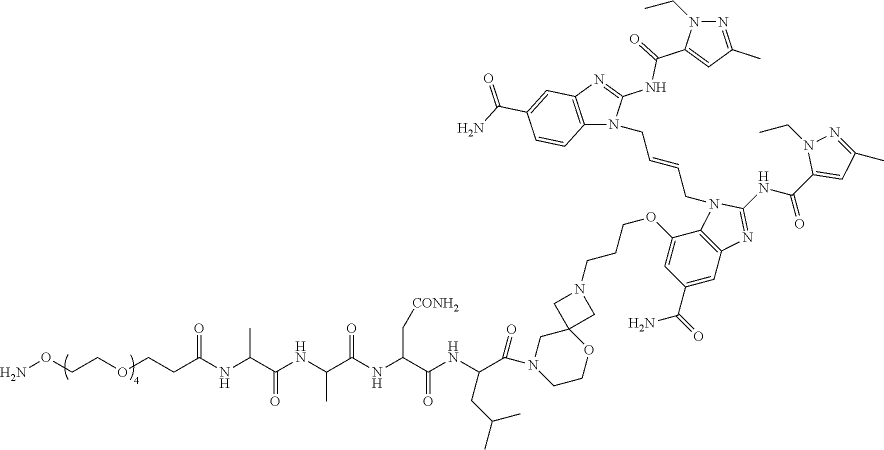 |
| LP- B6 | 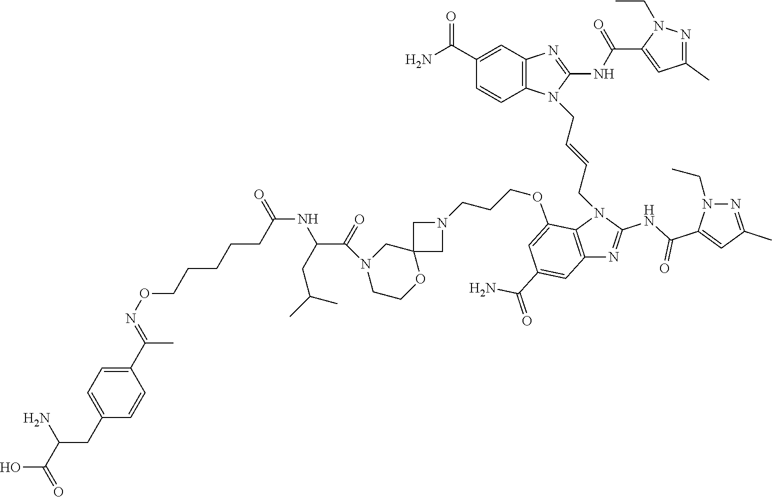 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)C(CC(C)C)NC(=O)CCCCCO/N=C(\C)c3ccc(CC(N)C(=O)O)cc3)CCO4)c21] |
| LP- B7 | 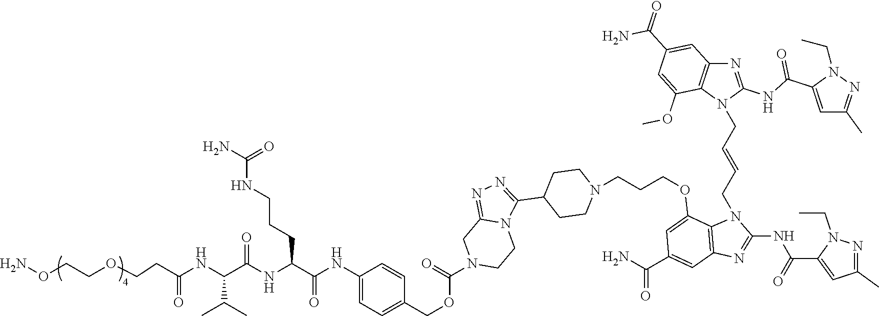 |
| LP- B8 | 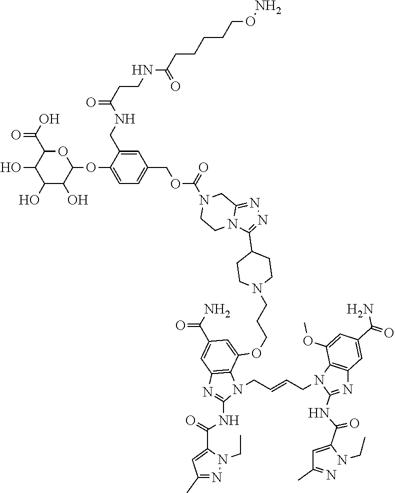 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(OC6OC(C(=O)O)C(O)C(O)C6O)c(CNC(=O)CCNC(=O)CCCCCON)c4)C5)CC3)c21] |
| LP- B9 | 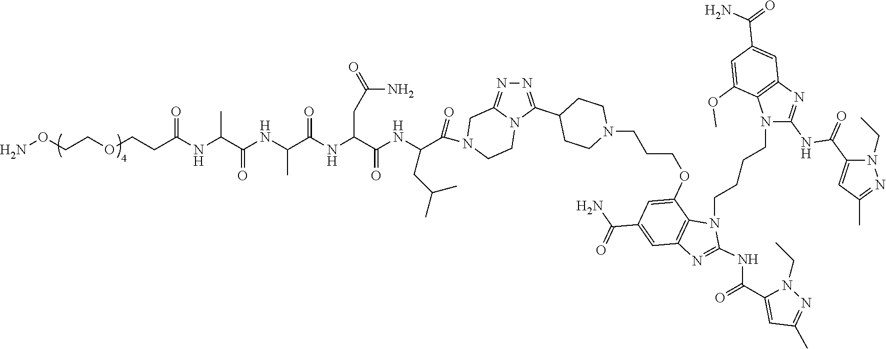 |
| LP- B10 | 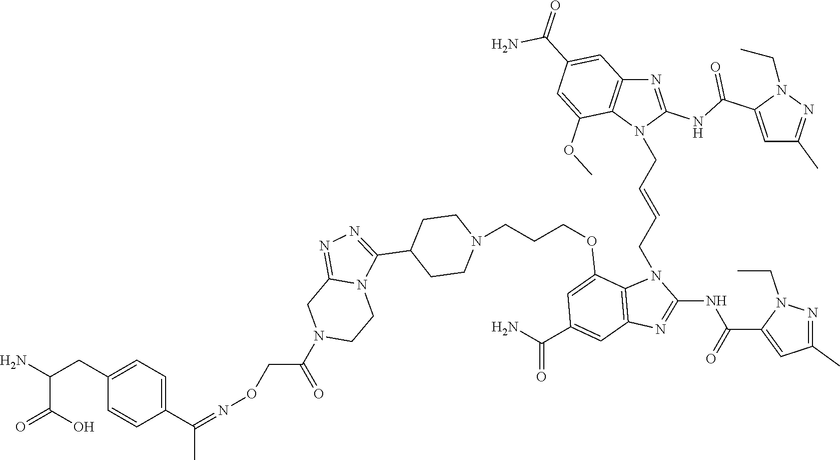 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(CC(N)C(=O)O)cc4)C5)CC3)c21] |
| LP- B11 | 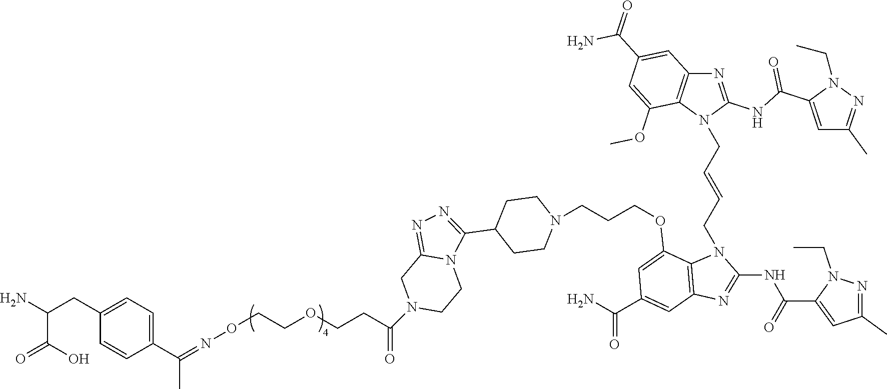 |
| LP- B12 | 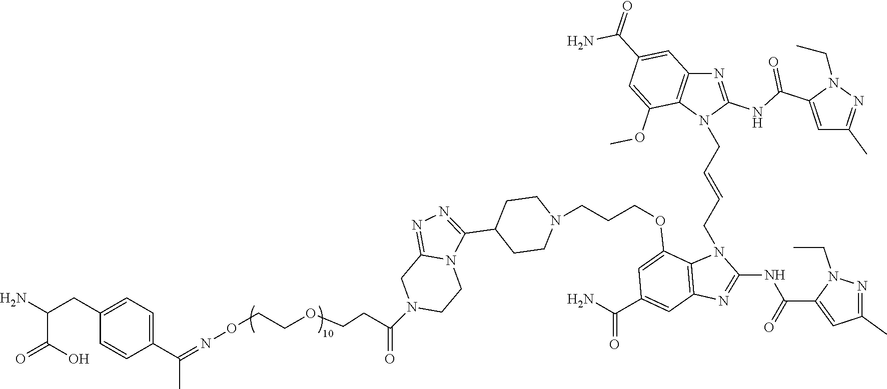 |
| LP- B13 | 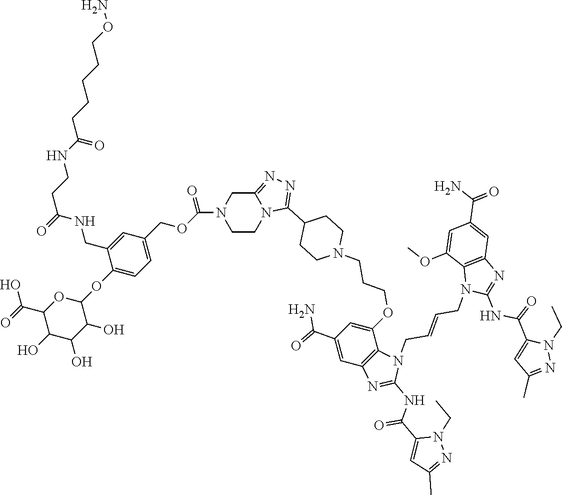 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(OC6OC(C(=O)O)C(O)C(O)C6O)c(CNC(=O)CCNC(=O)CCCCCON)c4)C5)CC3)c21] |
| LP- B14 | 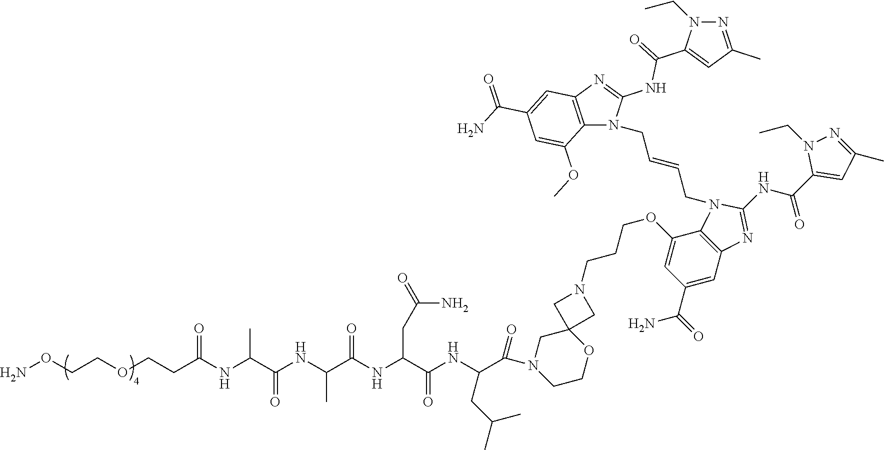 |
| LP- B15 | 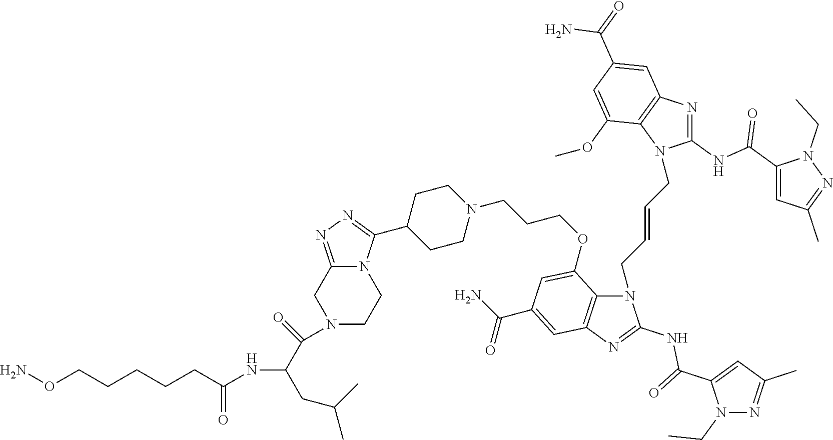 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)C(CC(C)C)NC(=O)CCCCCON)C5)CC3)c21] |
| LP- B16 | 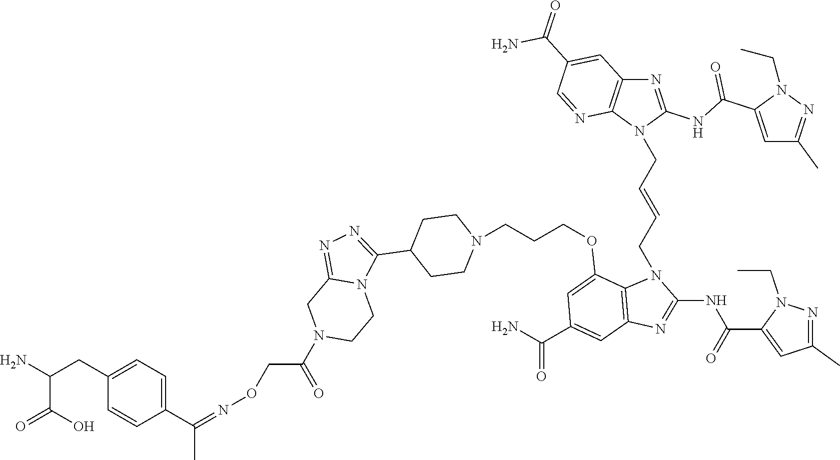 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(CC(N)C(=O)O)cc4)C5)CC3)c21] |
| LP- B17 | 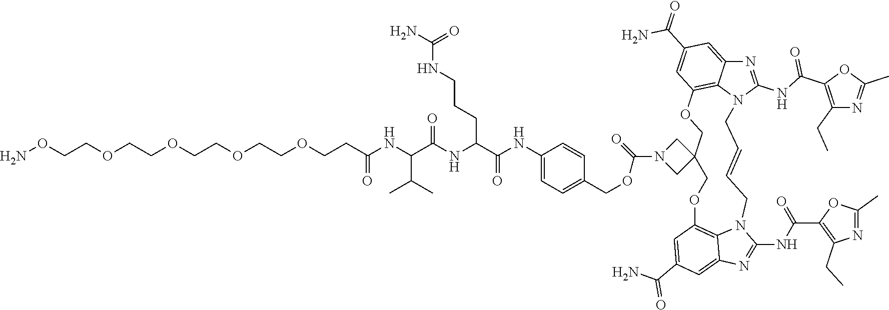 [SMILES: CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CO3)CN(C(=O)OCc2ccc(NC(=O)C(CCCNC(N)=O)NC(=O)C(NC(=O)CCOCCOCCOCCOCCON)C(C)C)cc2)C1] |
| LP- B18 | 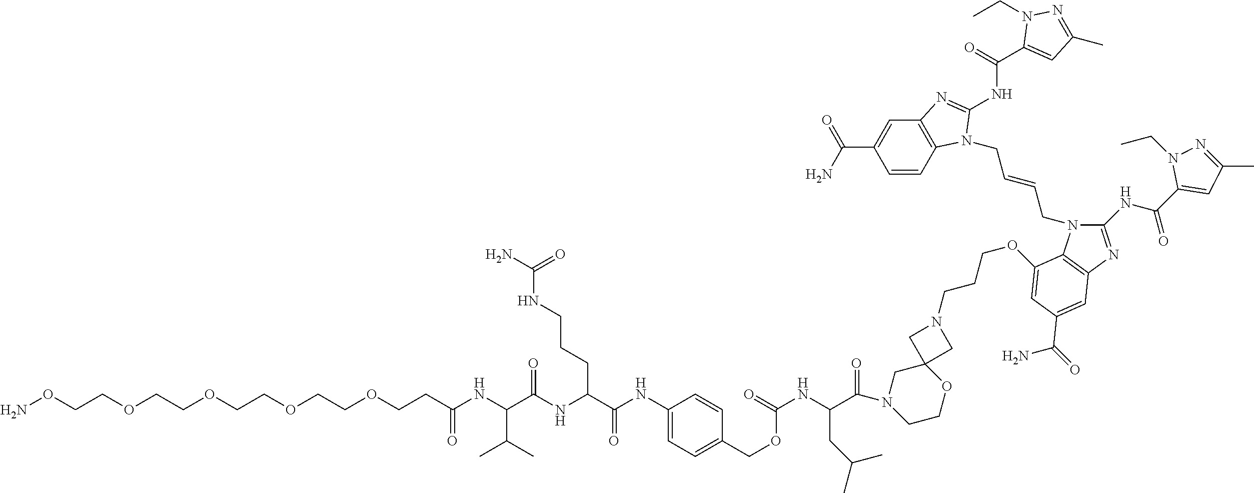 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)C(CC(C)C)NC(=O)OCc3ccc(NC(=O)C(CCCNC(N)=O)NC(=O)C(NC(=O)CCOCCOCCOCCOCCON)C(C)C)cc3)CCO4)c21] |
| LP- B19 | 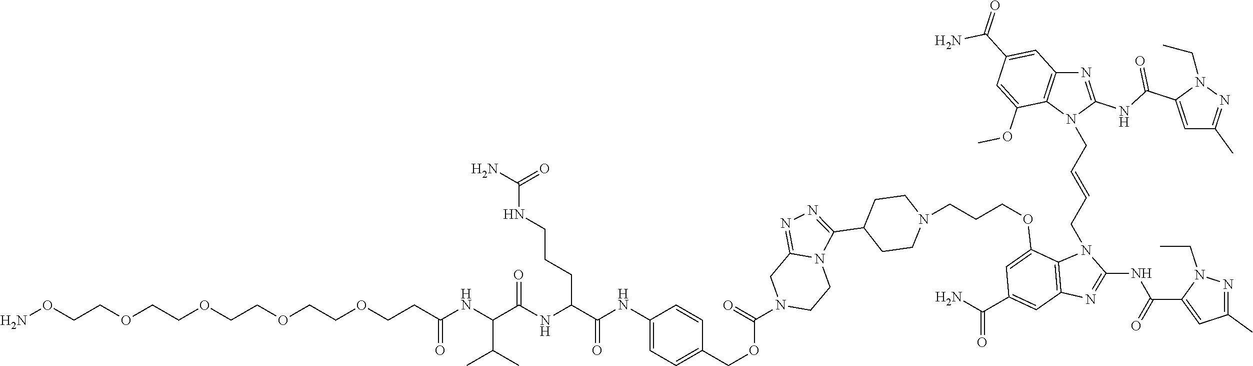 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(NC(=O)C(CCCNC(N)=O)NC(=O)C(NC(=O)CCOCCOCCOCCOCCON)C(C)C)cc4)C5)CC3)c21] |
| LP- B20 | 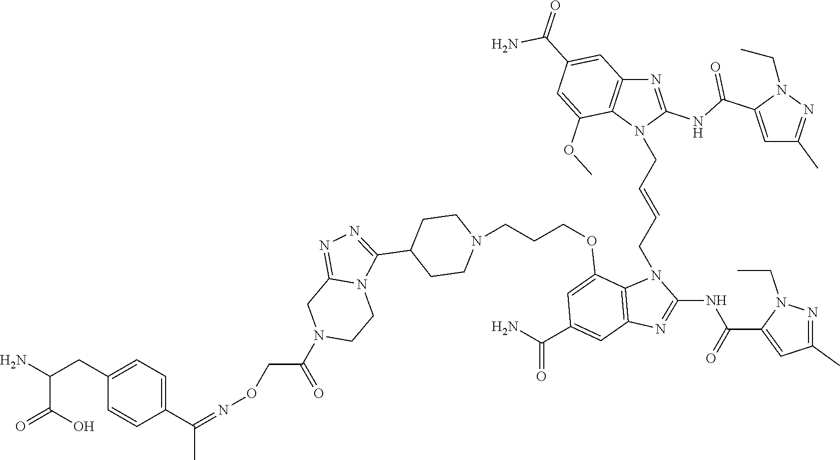 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(CC(N)C(=O)O)cc4)C5)CC3)c21] |
| LP- B21 | 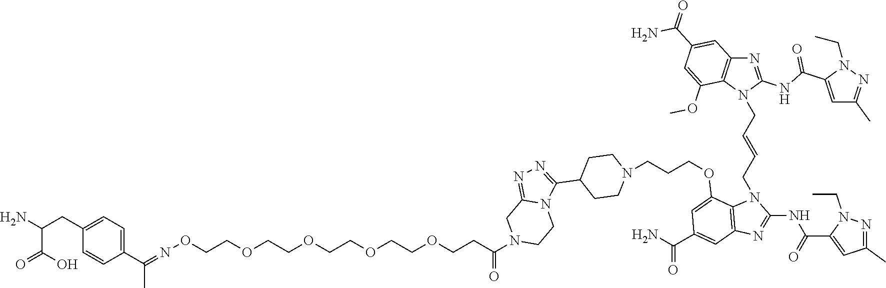 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCON=C(C)c4ccc(CC(N)C(=O)O)cc4)C5)CC3)c21] |
| LP- B22 | 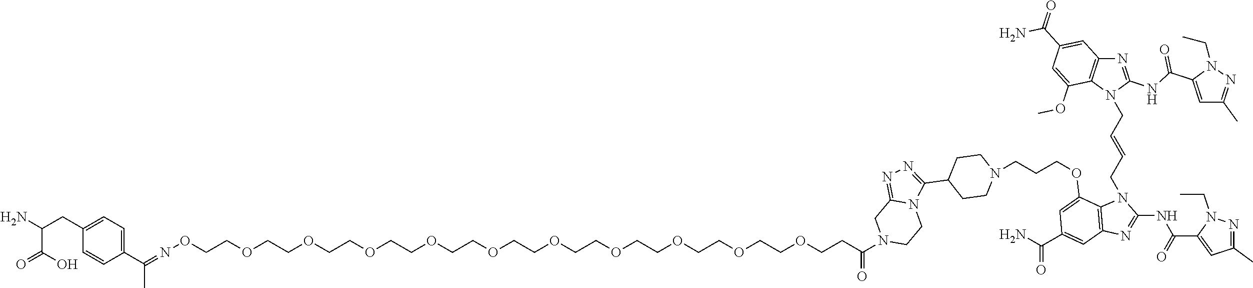 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCOCCOCCOCCOCCOCCOCCON=C(C)c4ccc(CC(N)C(=O)O)cc4)C5)CC3)c21] |
| LP- B23 | 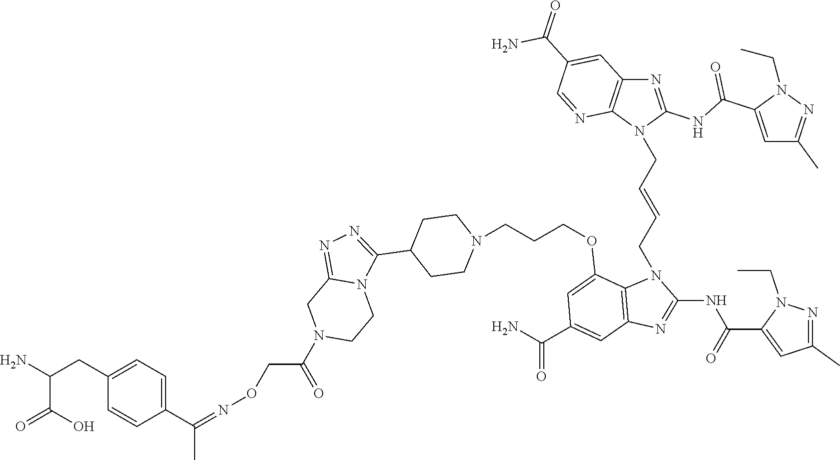 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(CC(N)C(=O)O)cc4)C5)CC3)c21] |
| LP- B24 | 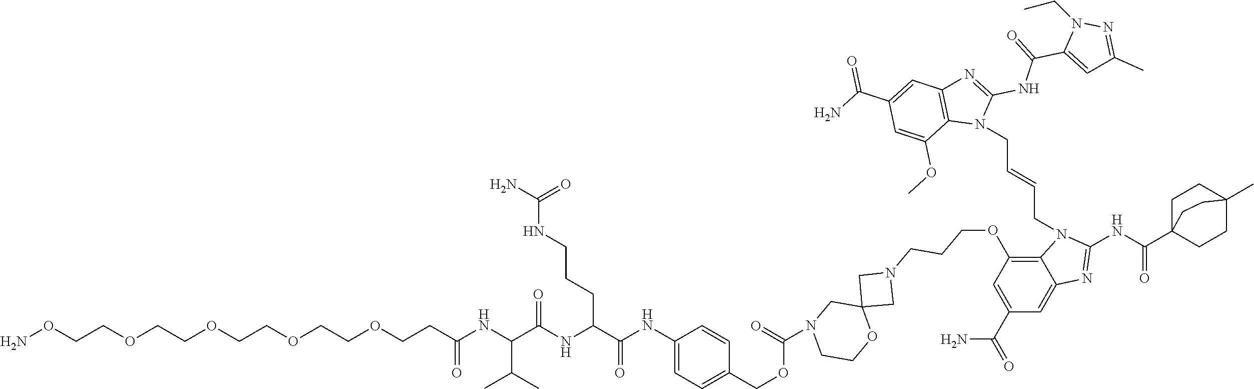 [SMILES: CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(C3)CN(C(=O)OCc3ccc(NC(=O)C(CCCNC(N)=O)NC(=O)C(NC(=O)CCOCCOCCOCCOCCON)C(C)C)cc3)CCO4)c21] |
| LP- B25 | 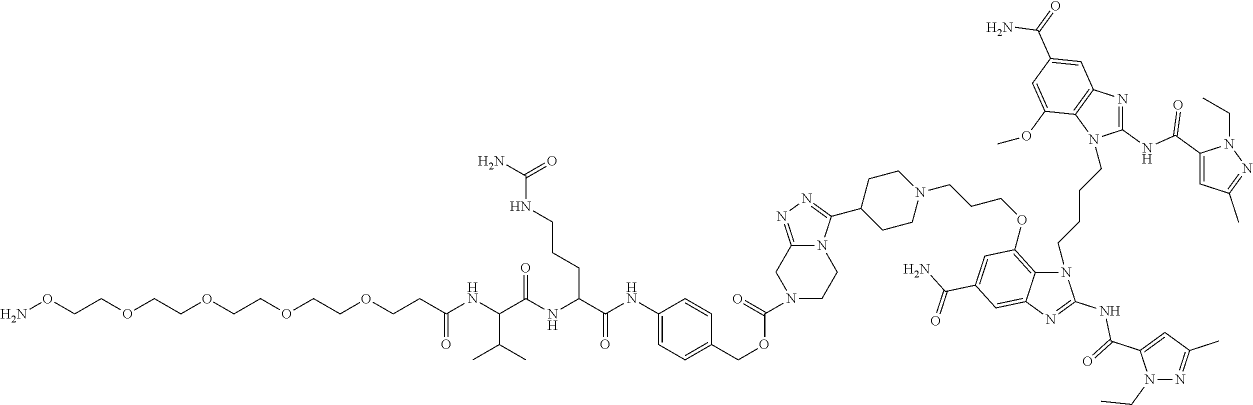 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCN(C(=O)OCc4ccc(NC(=O)C(CCCNC(N)=O)NC(=O)C(NC(=O)CCOCCOCCOCCOCCON)C(C)C)cc4)C5)CC3)c21] |
| LP- B26 | 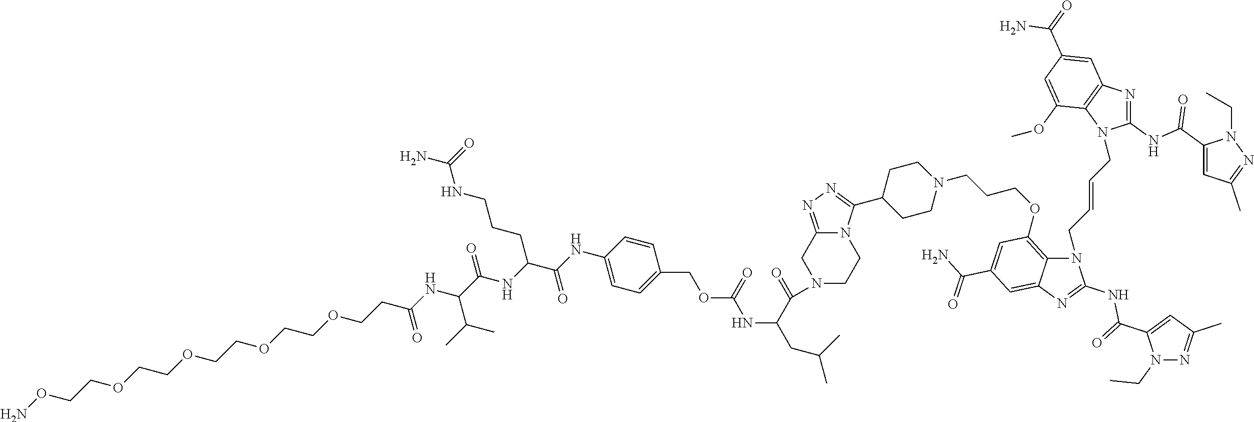 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)C(CC(C)C)NC(=O)OCc4ccc(NC(=O)C(CCCNC(N)=O)NC(=O)C(NC(=O)CCOCCOCCOCCOCCON)C(C)C)cc4)C5)CC3)c21] |
| LP- B27 | 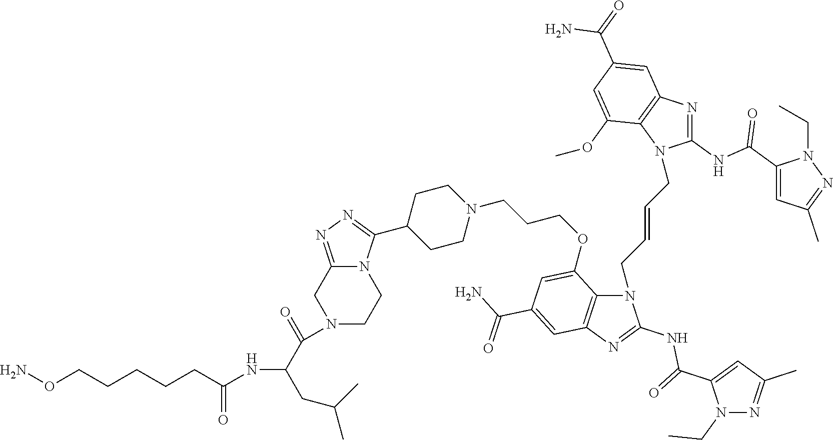 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)C(CC(C)C)NC(=O)CCCCCON)C5)CC3)c21] |
| LP- B28 | 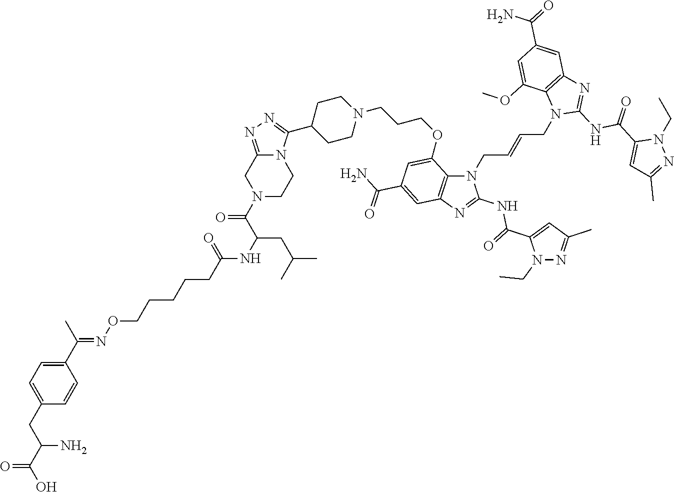 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)C(CC(C)C)NC(=O)CCCCCO/N=C(\C)c4ccc(CC(N)C(=O)O)cc4)C5)CC3)c21] |
| LP- B29 | 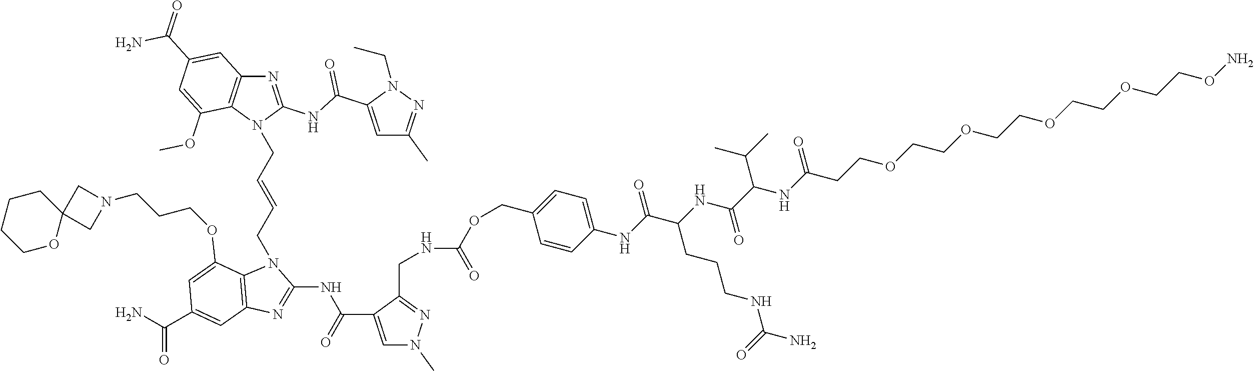 [SMILES: CCn1nc(C)cc1C(=O)Nc1nc2cc(C(N)=O)cc(OC)c2n1C/C=C/Cn1c(NC(=O)c2cn(C)nc2CNC(=O)OCc2ccc(NC(=O)C(CCCNC(N)=O)NC(=O)C(NC(=O)CCOCCOCCOCCOCCON)C(C)C)cc2)nc2cc(C(N)=O)cc(OCCCN3CC4(CCCCO4)C3)c21] |
| LP- B30 | 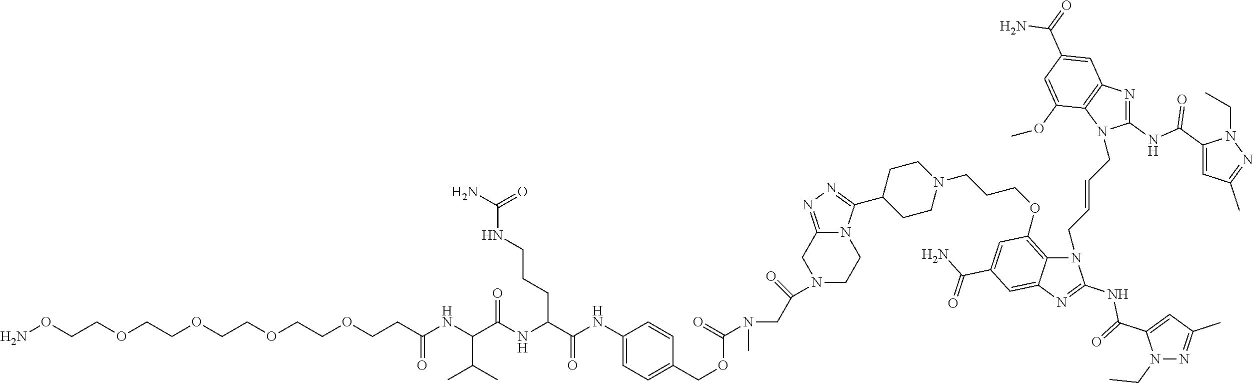 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CN(C)C(=O)OCc4ccc(NC(=O)C(CCCNC(N)=O)NC(=O)C(NC(=O)CCOCCOCCOCCOCCON)C(C)C)cc4)C5)CC3)c21] |
| LP- B31 | 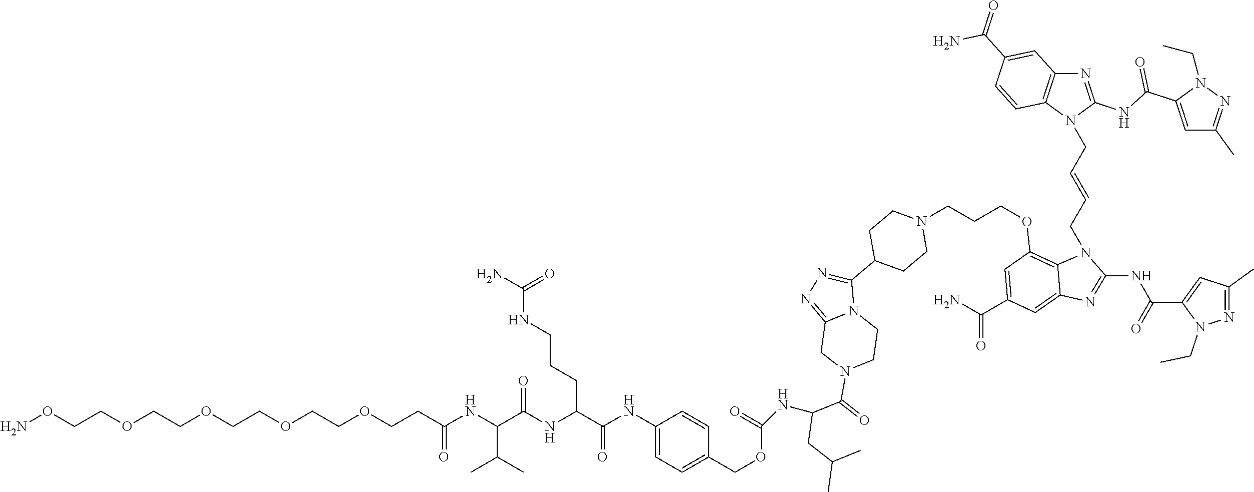 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)C(CC(C)C)NC(=O)OCc4ccc(NC(=O)C(CCCNC(N)=O)NC(=O)C(NC(=O)CCOCCOCCOCCOCCON)C(C)C)cc4)C5)CC3)c21] |

c. Compound Conjugates of Formula (CONJ-I), (CONJ-II), (CONJ-III), (CONJ-IV), and (CONJ-V)

Provided herein are compound conjugates comprising a compound of Formula (I), (II) (III), or (IV) wherein the compound of Formula (I), (II), (III) or (IV) is linked to a COMP optionally via a linker wherein COMP is a macromolecule. In one embodiment, the COMP is an antibody or antigen binding fragment thereof.

In one embodiment, the compound conjugate is of Formula (CONJ-IA) or Formula (CONJ-IB):

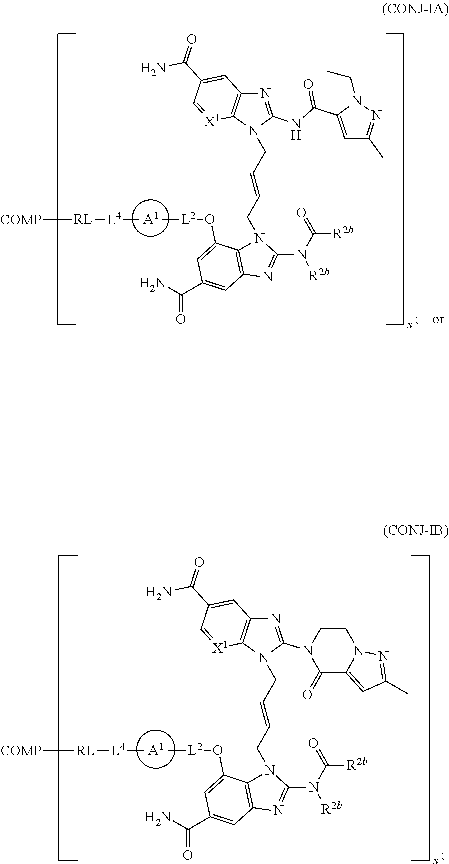

or a pharmaceutically acceptable salt or tautomer thereof.

In one embodiment of Formula (CONJ-IA), L² is —(CH₂)₃—; R^{1b} is hydrogen; and R^{2b} is

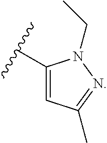 [SMILES: *c1cc(C)nn1CC]

In one embodiment of Formula (CONJ-IA), L² is —(CH₂)₃—; and R^{1b} and R^{2b} are joined together to form

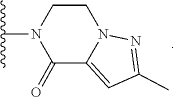 [SMILES: *N1CCn2nc(C)cc2C1=O]

In one embodiment of Formula (CONJ-IA), L² is —(CH₂)₃—; R^{1b} is hydrogen; and R^{2b} is

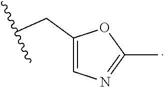 [SMILES: *Cc1cnc(C)o1]

In one embodiment of Formula (CONJ-IA), L² is —(CH₂)₃—; R^{1b} is hydrogen; and R^{2b} is

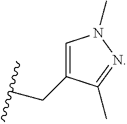 [SMILES: *Cc1cn(C)nc1C]

In one embodiment of Formula (CONJ-IA), L² is —(CH₂)₃—; R^{1b} is hydrogen; and R^{2b} is

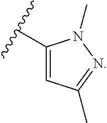 [SMILES: *c1cc(C)nn1C]

In one embodiment of Formula (CONJ-IA), L² is —(CH₂)₃—; R^{1b} is hydrogen; and R^{2b} is

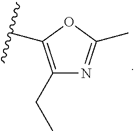 [SMILES: *c1oc(C)nc1CC]

In one embodiment of Formula (CONJ-IA), L² is —(CH₂)₃—; R^{1b} is hydrogen; and R^{2b} is

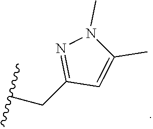 [SMILES: *Cc1cc(C)n(C)n1]

In one embodiment of Formula (CONJ-IA), L² is —(CH₂)₃—; R^{1b} is hydrogen; and R^{2b} is

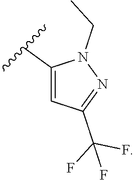 [SMILES: *c1cc(C(F)(F)F)nn1CC]

In one embodiment of Formula (CONJ-IA), L² is —(CH₂)₃—; R^{1b} is hydrogen; and R^{2b} is

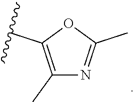 [SMILES: *c1oc(C)nc1C]

In one embodiment of Formula (CONJ-IA), L² is —(CH₂)₃—; R^{1b} is hydrogen; and R^{2b} is

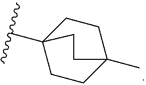 [SMILES: *C12CCC(C)(CC1)CC2]

In one embodiment of Formula (CONJ-IA), L² is —(CH₂)₃—; R^{1b} is hydrogen; and R^{2b} is

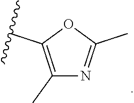 [SMILES: *c1oc(C)nc1C]

In one embodiment of Formula (CONJ-IA), L² is —(CH₂)₃—; R^{1b} is hydrogen; and R^{2b} is

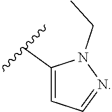 [SMILES: *c1ccnn1CC]

In one embodiment of Formula (CONJ-IA), L² is —(CH₂)₃—; R^{1b} is hydrogen; and R^{2b} is

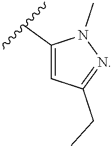 [SMILES: *c1cc(CC)nn1C]

In one embodiment of Formula (CONJ-IA), L² is —(CH₂)₃—; R^{1b} is hydrogen; and R^{2b} is

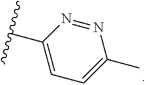 [SMILES: *c1ccc(C)nn1]

In one embodiment of Formula (CONJ-IA), L² is —(CH₂)₃—; R^{1b} is hydrogen; and R^{2b} is

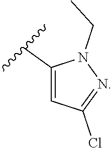 [SMILES: *c1cc(Cl)nn1CC]

In one embodiment of Formula (CONJ-IA), L² is —(CH₂)₃—; R^{1b} is hydrogen; and R^{2b} is

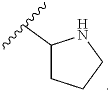 [SMILES: *C1CCCN1]

In one embodiment of Formula (CONJ-IA), L² is —(CH₂)₃—; R^{1b} is hydrogen; and R^{2b} is

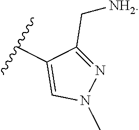 [SMILES: *c1cn(C)nc1CN]

In one embodiment of Formula (CONJ-IA), L² is —(CH₂)₃—; R^{1b} is hydrogen; and R^{2b} is

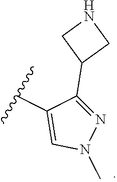 [SMILES: *c1cn(C)nc1C1CNC1]

In one embodiment of Formula (CONJ-IB), L² is —(CH₂)₃—; and R^{1b} and R^{2b} are joined together to form

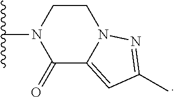 [SMILES: *N1CCn2nc(C)cc2C1=O]

In one embodiment of Formula (CONJ-IB), L² is —(CH₂)₃—; and R^{1b} and R^{2b} are joined together to form

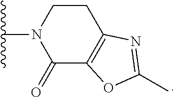 [SMILES: *N1CCc2nc(C)oc2C1=O]

In one embodiment, the compound conjugate is of Formula (CONJ-IIA), (CONJ-IIB), or (CONJ-IIC):

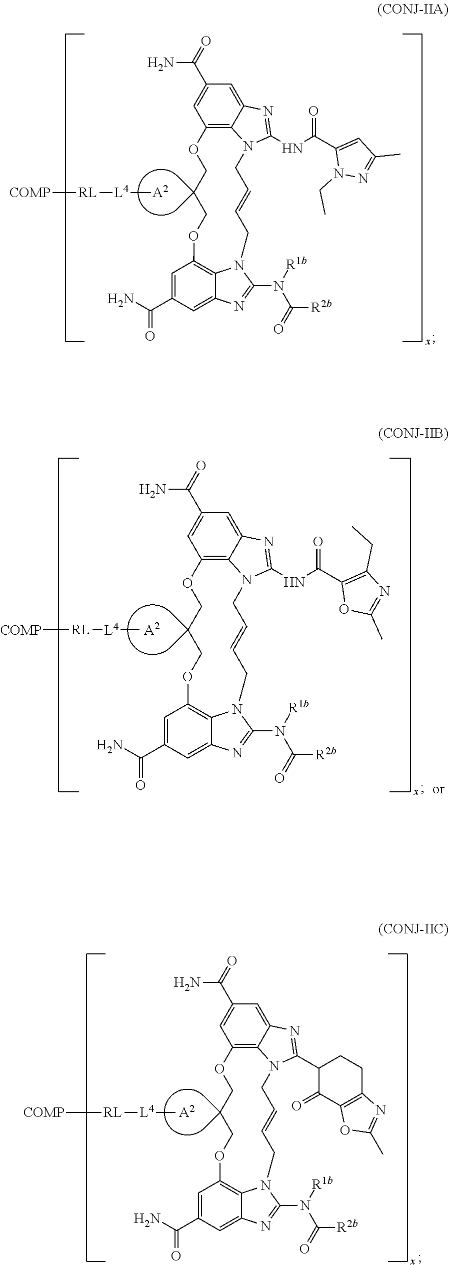

or a pharmaceutically acceptable salt or tautomer thereof, wherein

Ring A² is an optionally substituted C₃₋₁₂ carbocycle or an optionally substituted 3- to 12-membered heterocycle, wherein said optionally substituted C₃₋₁₂ carbocycle and optionally substituted 3- to 12-membered heterocycle are optionally substituted with one or more R⁵²;

In one embodiment of Formula (CONJ-IIA), R^{1b} is hydrogen; and R^{2b} is

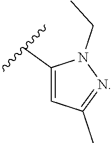 [SMILES: *c1cc(C)nn1CC]

In one embodiment of Formula (CONJ-IIA), and R^{1b} and R^{2b} are joined together to form

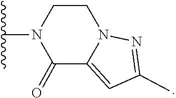 [SMILES: *N1CCn2nc(C)cc2C1=O]

In one embodiment of Formula (CONJ-IIB), R^{1b} is hydrogen; and R^{2b} is

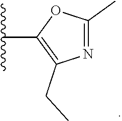 [SMILES: *c1oc(C)nc1CC]

In one embodiment of Formula (CONJ-IIB), R^{1b} is hydrogen; and R^{2b} is

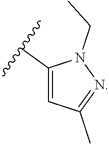 [SMILES: *c1cc(C)nn1CC]

In one embodiment of Formula (CONJ-IIC), R^{1b} is hydrogen; and R^{2b} is

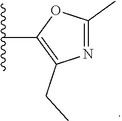 [SMILES: *c1oc(C)nc1CC]

In one embodiment of Formula (CONJ-IIC), and R^{1b} and R^{2b} are joined together to form

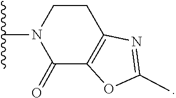 [SMILES: *N1CCc2nc(C)oc2C1=O]

In one embodiment, the compound conjugate is of Formula (CONJ-IIIA):

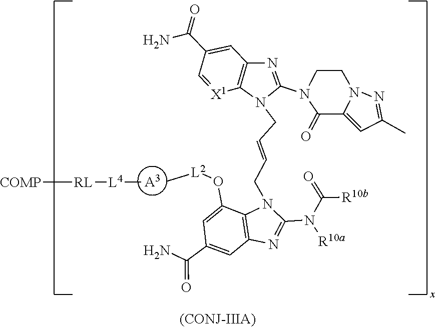

or a pharmaceutically acceptable salt or tautomer thereof.

In one embodiment of Formula (CONJ-IIIA), L² is —(CH₂)₃— and R^{10b} and R^{10a} are joined together to form

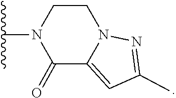 [SMILES: *N1CCn2nc(C)cc2C1=O]

In one embodiment, the compound conjugate is of Formula (CONJ-IVA):

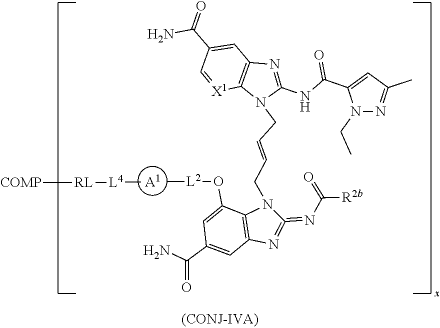

or a pharmaceutically acceptable salt or tautomer thereof.

In one embodiment of Formula (CONJ-IVA), L² is —(CH₂)₃— and R^{2b} is

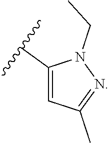 [SMILES: *c1cc(C)nn1CC]

In one embodiment, the compound conjugate is of Formula (CONJ-VA):

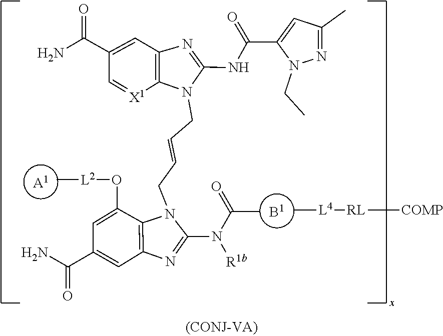

or a pharmaceutically acceptable salt or tautomer thereof

In one embodiment of Formula (CONJ-VA),

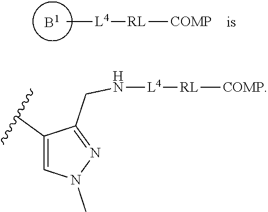

In one embodiment of Formula (CONJ-VA),

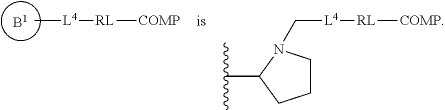

In one embodiment of Formula (CONJ-I), L¹ is —CH═CH—; R^{1a} and R^{1b} are both hydrogen; R^{2a} is

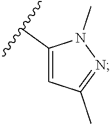 [SMILES: *c1cc(C)nn1C]

### and R^{2b} is

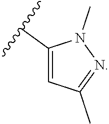 [SMILES: *c1cc(C)nn1C]

In one embodiment of Formula (CONJ-I), L¹ is —CH═CH—; R^{1a} and R^{1b} are both hydrogen; R^{2a} is

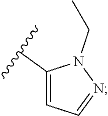 [SMILES: *c1ccnn1CC]

### and R^{2b} is

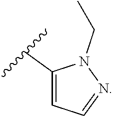 [SMILES: *c1ccnn1CC]

In one embodiment of Formula (CONJ-I), L¹ is —CH═CH—; R^{1a} and R^{1b} are both hydrogen; R^{2a} is

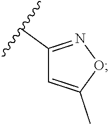 [SMILES: *c1cc(C)on1]

### and R^{2b} is

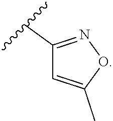 [SMILES: *c1cc(C)on1]

In one embodiment of Formula (CONJ-I), L¹ is —CH═CH—; R^{1a} and R^{1b} are both hydrogen; R^{2a} is

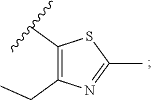 [SMILES: *c1sc(C)nc1CC]

### and R^{2b} is

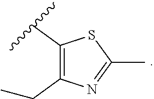 [SMILES: *c1sc(C)nc1CC]

In one embodiment of Formula (CONJ-I), L¹ is —CH═CH—; R^{1a} and R^{1b} are both hydrogen; R^{2a} is

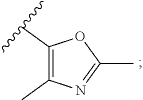 [SMILES: *c1oc(C)nc1C]

### and R^{2b} is

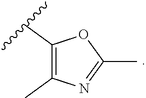 [SMILES: *c1oc(C)nc1C]

In one embodiment of Formula (CONJ-I), L¹ is —CH₂CH₂—; R^{1a} and R^{1b} are both hydrogen; R^{2a} is

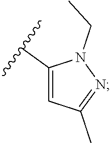 [SMILES: *c1cc(C)nn1CC]

### and R^{2b} is

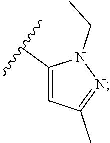 [SMILES: *c1cc(C)nn1CC]

In one embodiment of Formula (CONJ-I), L¹ is —CH═CH—; R²⁰ is hydrogen; R^{1a} and R^{1b} are both hydrogen; and R^{2a} and R^{2b} are both

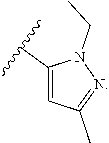 [SMILES: *c1cc(C)nn1CC]

In one embodiment of Formula (CONJ-II), L¹ is —CH═CH—; R^{1a} and R^{1b} are both hydrogen and R^{2a} and R^{2b} are both

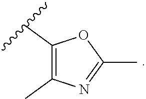 [SMILES: *c1oc(C)nc1C]

In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is an optionally substituted bridged, fused, or spirocyclic bicyclic heterocycle comprising at least one N atom and at least one O atom wherein the heterocycle is optionally substituted with one or more R⁵³. In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is an optionally substituted 3- to 12-membered bridged, fused, or spirocyclic bicyclic heterocycle comprising at least one nitrogen atom, including the nitrogen bound to L², and at least one oxygen atom. In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is an optionally substituted 8- to 12-membered bridged, fused, or spirocyclic bicyclic heterocycle comprising at least one nitrogen atom, including the nitrogen bound to L², and at least one oxygen atom. In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is an optionally substituted 3- to 12-membered N-linked bridged, fused, or spirocyclic bicyclic heterocycle comprising at least one nitrogen atom, including the nitrogen bound to L², and at least one oxygen atom. In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is an optionally substituted 8- to 12-membered N-linked bridged, fused, or spirocyclic bicyclic heterocycle comprising at least one nitrogen atom, including the nitrogen bound to L², and at least one oxygen atom. In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is an optionally substituted 3- to 12-membered N-linked spirocyclic bicyclic heterocycle comprising at least one nitrogen atom, including the nitrogen bound to L², and at least one oxygen atom. In one embodiment of Formula (CONJ-I), (CONJ-IA) (CONJ-IB), (CONJ-IV), or (CONJ-IVA), including any of the foregoing, Ring A¹ is an optionally substituted 3- to 12-membered N-linked spirocyclic bicyclic heterocycle comprising two nitrogen atoms, including the nitrogen bound to L² and a nitrogen bound to L⁴, and one oxygen atom.

In one embodiment of Formula (CONJ-I), (CONJ-IA) (CONJ-IB), (CONJ-IV), or (CONJ-IVA), including any of the foregoing, Ring A¹ is selected from

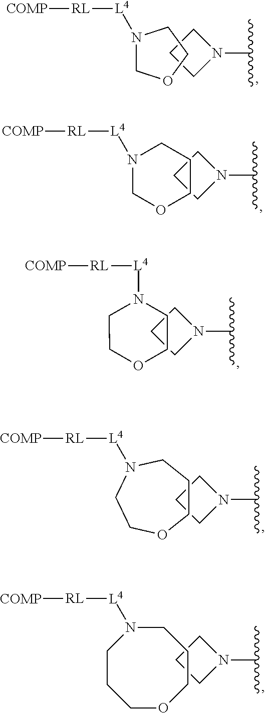

wherein L⁴, RL, and COMIP are as defined herein.

In one embodiment of Formula (CONJ-I), (CONJ-IA) (CONJ-IB), (CONJ-IV), or (CONJ-IVA), including any of the foregoing, Ring A¹ is selected from

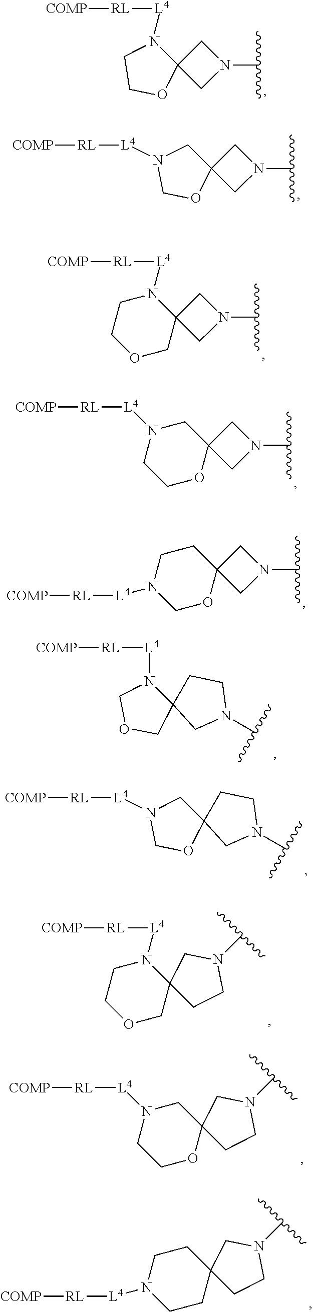

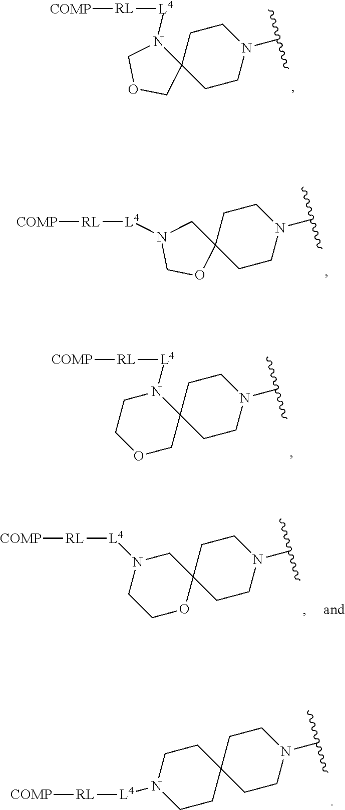

In one embodiment of Formula (CONJ-I), (CONJ-IA) (CONJ-IB), (CONJ-IV), or (CONJ-IVA), including any of the foregoing, Ring A¹ is selected from

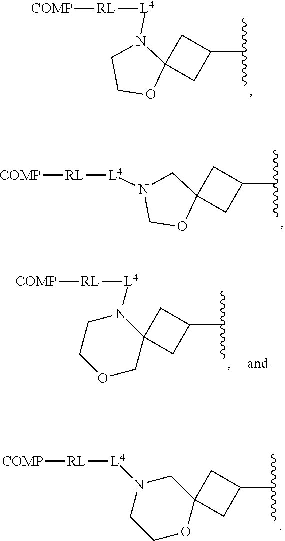

In one embodiment of Formula (CONJ-I), (CONJ-IA) (CONJ-IB), (CONJ-IV), or (CONJ-IVA), including any of the foregoing, Ring A¹ is selected from

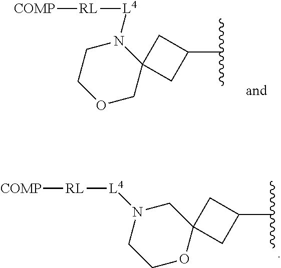

In one embodiment of Formula (CONJ-I), (CONJ-IA) (CONJ-IB), (CONJ-IV) or (CONJ-IVA), including any of the foregoing, Ring A¹ is

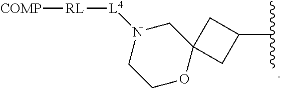

In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is a 3- to 12-membered heterocycle substituted with R⁴. In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is a 8- to 12-membered heterocycle substituted with R⁴.

In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is a N-linked monocyclic 3- to 12-membered heterocycle comprising the N to which the ring is attached and a second NH and wherein the heterocycle is substituted with R⁴. In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is a N-linked monocyclic 8- to 12-membered heterocycle comprising the N to which the ring is attached and a second NH and wherein the heterocycle is substituted with R⁴.

In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is selected from

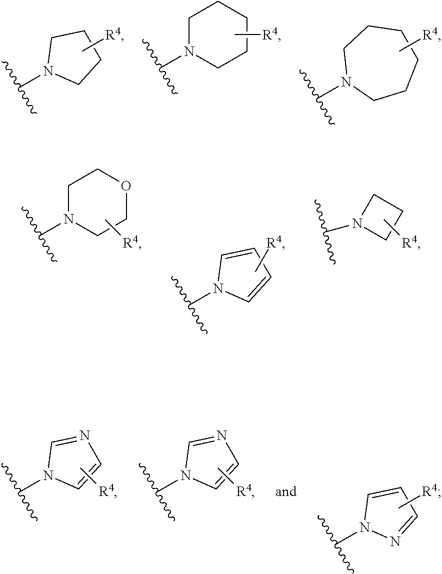

wherein

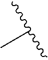

is the point of attachment to the rest of the compound.

In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is selected from

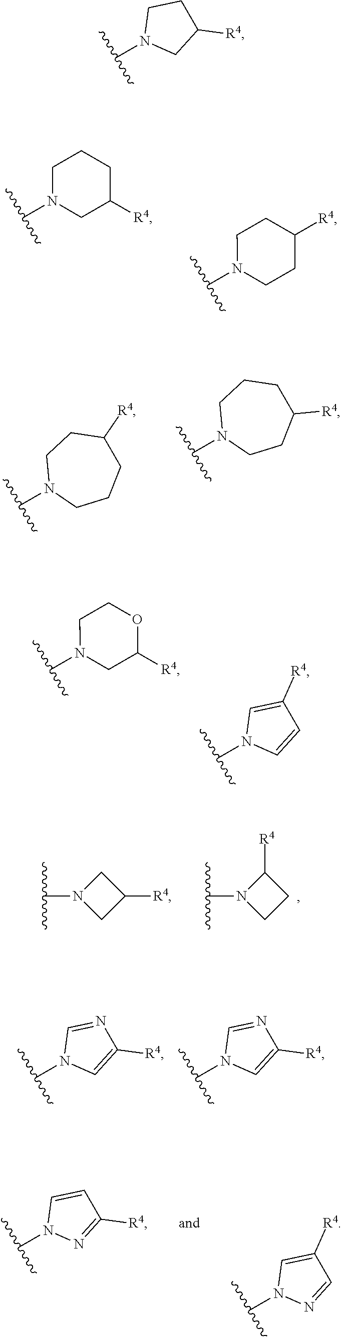

In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is selected from

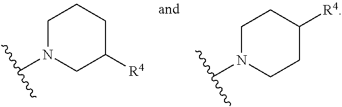

In one embodiment of Formula (CONJ-I), (CONJ-IA) (CONJ-IB), (CONJ-IV), (CONJ-IVA), (CONJ-V), or (CONJ-VA), including any of the foregoing, Ring A¹ is

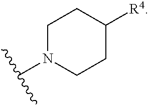

In one embodiment of Formula (CONJ-I), (CONJ-IA) (CONJ-IB), (CONJ-IV), (CONJ-IVA), (CONJ-V), or (CONJ-VA), including any of the foregoing, R⁴ is a 3- to 12-membered bridged or fused bicyclic heterocycle comprising 1, 2, 3, or 4 heteroatoms selected from N, S, and O. In one embodiment of Formula (CONJ-I), (CONJ-IA) (CONJ-IB), (CONJ-IV), (CONJ-IVA), (CONJ-V), or (CONJ-VA), including any of the foregoing, R⁴ is a 8- to 12-membered bridged or fused bicyclic heterocycle comprising 1, 2, 3, or 4 heteroatoms selected from N, S, and O. In one embodiment of Formula (CONJ-I), (CONJ-IA) (CONJ-IB), (CONJ-IV), (CONJ-IVA), (CONJ-V), or (CONJ-VA), including any of the foregoing, R⁴ is a 5-5 fused ring system, a 5-6 fused ring system, a 6-6 fused ring system, or a 5-7 fused ring system comprising 1, 2, 3, or 4 heteroatoms selected from N, S, and O. In one embodiment of Formula (CONJ-I), (CONJ-IA) (CONJ-IB), (CONJ-IV), (CONJ-IVA), (CONJ-V), or (CONJ-VA), including any of the foregoing, R⁴ is a 5-6 fused ring system comprising 1, 2, 3, or 4 heteroatoms selected from N, S, and O.

In one embodiment of Formula (CONJ-I), (CONJ-IA) (CONJ-IB), (CONJ-IV), or (CONJ-IVA), including any of the foregoing, R⁴ is a 3- to 12-membered bridged or fused bicyclic heterocycle comprising 1, 2, 3, or 4 heteroatoms selected from N, S, and O wherein at least one heteroatom is a N bound to -L⁴-RL-COMP. In one embodiment of Formula (CONJ-I), (CONJ-IA) (CONJ-IB), (CONJ-IV), or (CONJ-IVA), including any of the foregoing, R⁴ is a 5-5 fused ring system, a 5-6 fused ring system, a 6-6 fused ring system, or a 5-7 fused ring system comprising 1, 2, 3, or 4 heteroatoms selected from N, S, and O wherein at least one heteroatom is a N bound to -L⁴-RL-COMP. In one embodiment of Formula (CONJ-I), (CONJ-IA) (CONJ-IB), (CONJ-IV), or (CONJ-IVA), including any of the foregoing, R⁴ is a 5-6 fused ring system comprising 1, 2, 3, or 4 heteroatoms selected from N, S, and O wherein at least one heteroatom is a N bound to -L⁴-RL-COMP.

In one embodiment of Formula (CONJ-I), (CONJ-IA) (CONJ-IB), (CONJ-IV), or (CONJ-IVA), including any of the foregoing, R⁴ is

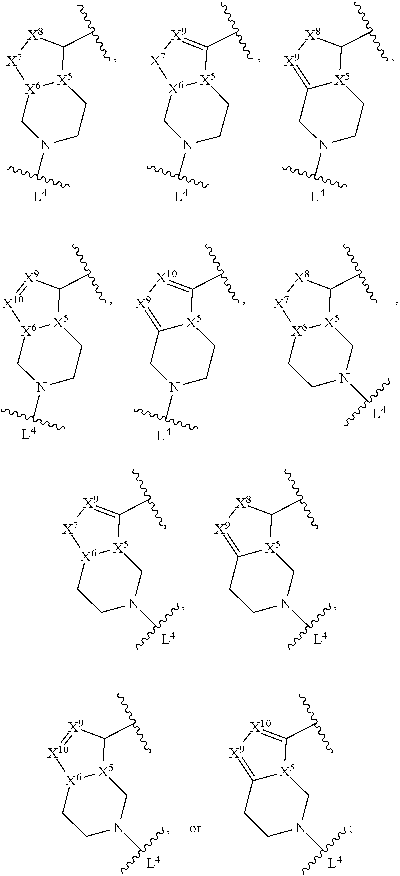

wherein X⁵, X⁶, X⁹, X¹⁰ are independently N or CR¹¹;
X⁷ and X⁸ are independently NH, O, or CHR¹¹;
R¹¹ is hydrogen or C₁₋₆ alkyl;
R⁵ is as defined herein; and

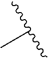

is the point of attachment to the rest of the compound and

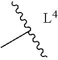

is a bond to L⁴;
wherein if four of any one of X⁵—X¹⁰ are present, at least one is CR¹¹ or CHR¹¹.

In one embodiment of Formula (CONJ-I), (CONJ-IA) (CONJ-IB), (CONJ-IV), or (CONJ-IVA), including any of the foregoing, R⁴ is

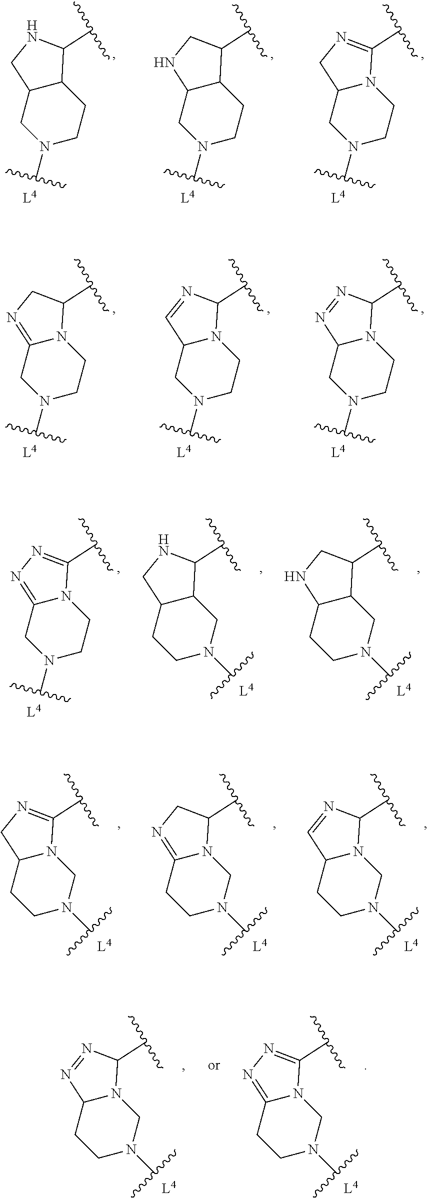

In one embodiment of Formula (CONJ-I), (CONJ-IA) (CONJ-IB), (CONJ-IV), or (CONJ-IVA), including any of the foregoing, R⁴ is

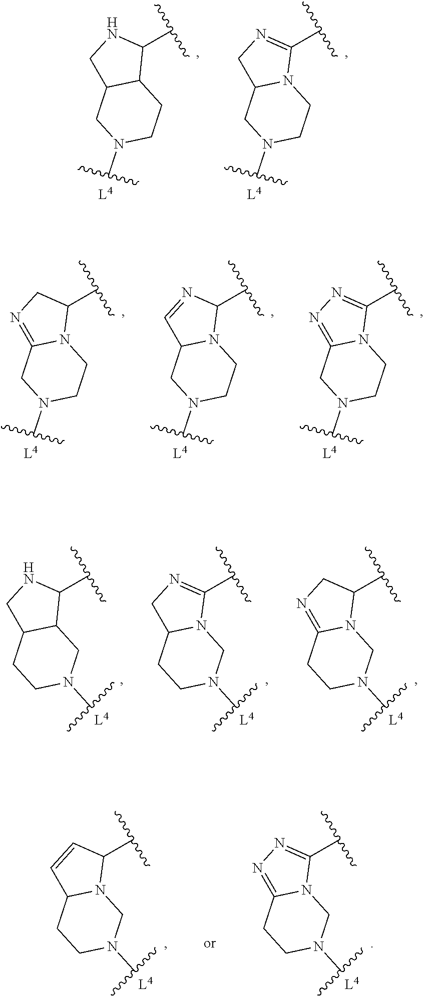

In one embodiment of Formula (CONJ-I), (CONJ-IA) (CONJ-IB), (CONJ-IV), or (CONJ-IVA), including any of the foregoing, R⁴ is

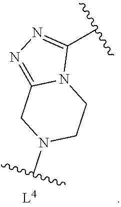 [SMILES: *c1nnc2n1CCN(*)C2]

In one embodiment of Formula (CONJ-I), (CONJ-IA) (CONJ-IB), (CONJ-IV), or (CONJ-IVA), including any of the foregoing, Ring A¹ is

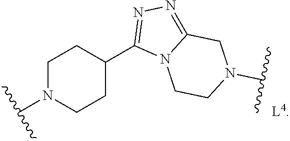 [SMILES: *N1CCC(c2nnc3n2CCN(*)C3)CC1]

In one embodiment of Formula (CONJ-III) or (CONJ-IIIA), including any of the foregoing, Ring A³ is an optionally substituted 3- to 12-membered heterocycle optionally substituted with one or more R⁵³. In one embodiment of Formula (CONJ-III) or (CONJ-IIIA), including any of the foregoing, Ring A³ is an optionally substituted 8- to 12-membered heterocycle optionally substituted with one or more R⁵³.

In one embodiment of Formula (CONJ-III) or (CONJ-IIIA), including any of the foregoing, Ring A³ is a N-linked monocyclic 3- to 12-membered heterocycle comprising a N bound to L² and a N bound to L⁴-RL-COMP and wherein the heterocycle is optionally substituted with one or more R⁵³. In one embodiment of Formula (CONJ-III) or (CONJ-IIIA), including any of the foregoing, Ring A³ is a N-linked monocyclic 8- to 12-membered heterocycle comprising a N bound to L² and a N bound to L⁴-RL-COMP and wherein the heterocycle is optionally substituted with one or more R⁵³.

In one embodiment of Formula (LP-III) or (LP-IIIA), including any of the foregoing, Ring A³ is

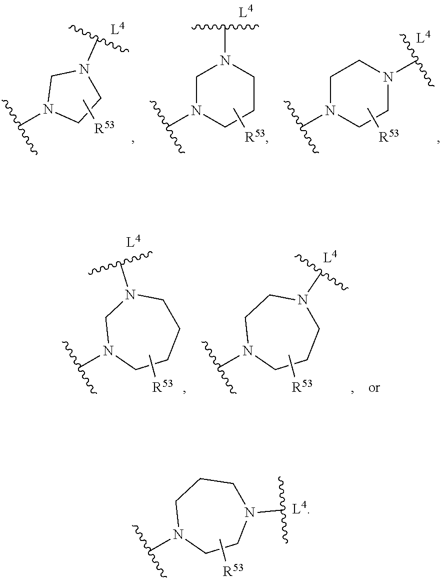

In one embodiment of Formula (CONJ-III) or (CONJ-IIIA), including any of the foregoing, Ring A³ is

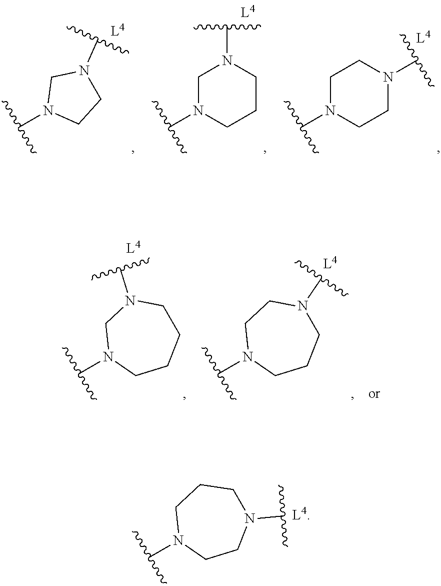 [SMILES: *N1CCN(*)C1 ; *N1CCCN(*)C1 ; *N1CCN(*)CC1 ; *N1CCCCN(*)C1 ; *N1CCCN(*)CC1 ; *N1CCCN(*)CC1]

In one embodiment of Formula (CONJ-III) or (CONJ-IIIA), including any of the foregoing, Ring A³ is

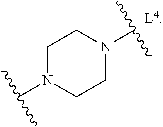

In one embodiment of Formula (CONJ-II), (CONJ-IIA), (CONJ-IIB), or (CONJ-IIC), including any of the foregoing, L⁶ is a linker comprising C₁₋₃ alkyl. In one embodiment of Formula (CONJ-II), (CONJ-IIA), (CONJ-IIB), or (CONJ-IIC), including any of the foregoing, L⁶ is a linker comprising C₃₋₆ alkyl. In one embodiment of Formula (CONJ-II), (CONJ-IIA), (CONJ-IIB), or (CONJ-IIC), including any of the foregoing, L⁶ is a linker comprising C₃ alkyl.

In one embodiment of Formula (CONJ-II), (CONJ-IIA), (CONJ-IIB), or (CONJ-IIC), including any of the foregoing, L⁶ is a linker comprising C₁₋₃ alkyl substituted with R^{28a} and R^{28b} wherein R^{28a} and R^{28b} are joined together with the atoms to which they are attached to form a C₃₋₁₂ carbocycle substituted with -L⁴-RL-COMP and further optionally substituted with one or more R⁵². In one embodiment of Formula (CONJ-II), (CONJ-IIA), (CONJ-IIB), or (CONJ-IIC), including any of the foregoing, L⁶ is a linker comprising C₁₋₃ alkyl substituted with R^{28a} and R^{28b} wherein R^{28a} and R^{28b} are joined together with the atoms to which they are attached to form a C₃₋₆ carbocycle substituted with -L⁴-RL-COMP and further optionally substituted with one or more R⁵²

In one embodiment of Formula (CONJ-II), (CONJ-IIA), (CONJ-IIB), or (CONJ-IIC), including any of the foregoing, L⁶ is a linker comprising C₁₋₃ alkyl substituted with R^{28a} and R^{28b} wherein R^{28a} and R^{28b} are joined together with the atoms to which they are attached to form a 3- to 12-membered heterocycle substituted with -L⁴-RL-COMP and further optionally substituted with one or more R⁵². In one embodiment of Formula (CONJ-II), (CONJ-IIA), (CONJ-IIB), or (CONJ-IIC), including any of the foregoing, L⁶ is a linker comprising C₁₋₃ alkyl substituted with R^{28a} and R^{28b} wherein R^{28a} and R^{28b} are joined together with the atoms to which they are attached to form a 4- to 6-membered heterocycle substituted with -L⁴-RL-COMP and further optionally substituted with one or more R⁵². In one embodiment of Formula (CONJ-II), (CONJ-IIA), (CONJ-IIB), or (CONJ-IIC), including any of the foregoing, L⁶ is a linker comprising C₁₋₃ alkyl substituted with R^{28a} and R^{28b} wherein R^{28a} and R^{28b} are joined together with the atoms to which they are attached to form a 3- to 12-membered heterocycle comprising a N bound to -L⁴-RL-COMP and optionally substituted with one or more R⁵². In one embodiment of Formula (CONJ-II), (CONJ-IIA), (CONJ-IIB), or (CONJ-IIC), or (LP-IIC), including any of the foregoing, L⁶ is a linker comprising C₁₋₃ alkyl substituted with R^{28a} and R^{28b} wherein R^{28a} and R^{28b} are joined together with the atoms to which they are attached to a 3- to 12-membered heterocycle comprising a N bound to -L⁴-RL-COMP. In one embodiment of Formula (CONJ-II), (CONJ-IIA), (CONJ-IIB), or (CONJ-IIC), or (LP-IIC), including any of the foregoing, L⁶ is a linker comprising C₁₋₃ alkyl substituted with R^{28a} and R^{28b} wherein R^{28a} and R^{28b} are joined together with the atoms to which they are attached to a 6- to 6-membered heterocycle comprising a N bound to -L⁴-RL-COMP.

In one embodiment of Formula (CONJ-II), (CONJ-IIA), (CONJ-IIB), or (CONJ-IIC), including any of the foregoing, R^{28a} and R^{28b} are joined together with the atoms to which they are attached to form

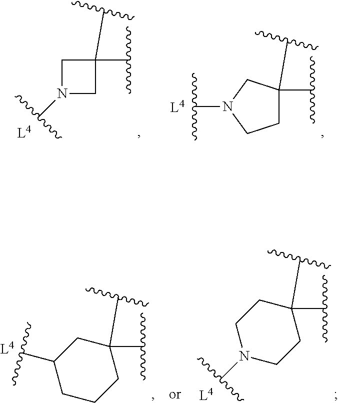

wherein

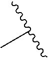

is the point of attachment to the rest of the compound and

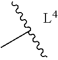

is a bond to L⁴. In one embodiment of Formula (CONJ-II), (CONJ-IIA), (CONJ-IIB), or (CONJ-IIC), including any of the foregoing, R^{28a} and R^{28b} are joined together with the atoms to which they are attached to form

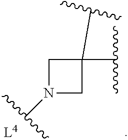

In one embodiment of Formula (CONJ-V) or (CONJ-VA), including any of the foregoing, Ring A¹ is selected from

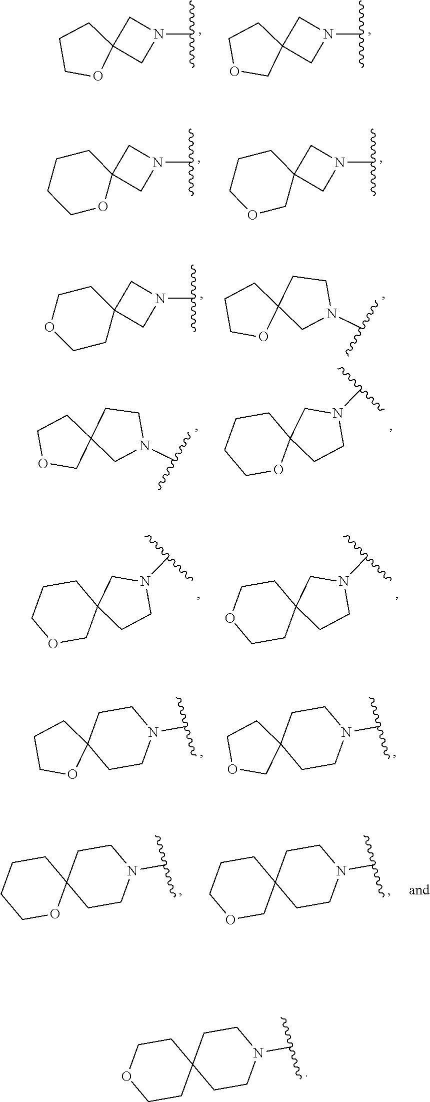

In one embodiment of Formula (CONJ-V) or (CONJ-VA), including any of the foregoing, Ring A¹ is

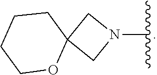 [SMILES: *N1CC2(CCCCO2)C1]

In one embodiment of Formula (CONJ-V) or (CONJ-VA), including any of the foregoing, Ring B¹ is an optionally substituted C₃₋₁₂ carbocycle optionally substituted with one or more R⁵². In one embodiment of Formula (CONJ-V) or (CONJ-VA), including any of the foregoing, Ring B¹ is an optionally substituted C₆₋₁₂ carbocycle optionally substituted with one or more R⁵². In one embodiment of Formula (CONJ-V) or (CONJ-VA), including any of the foregoing, Ring B¹ is an optionally substituted 3- to 12-membered heterocycle optionally substituted with one or more R⁵². In one embodiment of Formula (CONJ-V) or (CONJ-VA), including any of the foregoing, Ring B¹ is an optionally substituted 5- to 6-membered heterocycle optionally substituted with one or more R⁵². In one embodiment of Formula (CONJ-V) or (CONJ-VA), including any of the foregoing, Ring B¹ is an optionally substituted 3- to 12-membered heterocycle comprising at least a N bound to -L⁴-RL-COMP and optionally substituted with one or more R⁵². In one embodiment of Formula (CONJ-V) or (CONJ-VA), including any of the foregoing, Ring B¹ is an optionally substituted 4- to 6-membered heterocycle comprising at least a N bound to -L⁴-RL-COMP and optionally substituted with one or more R⁵². In one embodiment of Formula (CONJ-V) or (CONJ-VA), including any of the foregoing, Ring B¹ is a 3- to 12-membered heterocycle comprising at least a N bound to -L⁴-RL-COMP. In one embodiment of Formula (CONJ-V) or (CONJ-VA), including any of the foregoing, Ring B¹ is a 4- to 6-membered heterocycle comprising at least a N bound to -L⁴-RL-COMP.

In one embodiment of Formula (CONJ-V) or (CONJ-VA), including any of the foregoing, Ring B¹ is selected from the group consisting of

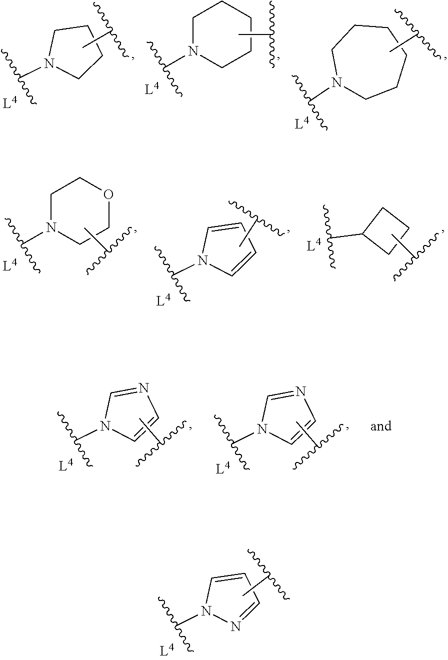

wherein

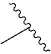

is the point of attachment to the rest of the compound and

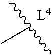

is a bond to L⁴.

In one embodiment of Formula (CONJ-V) or (CONJ-VA), including any of the foregoing, Ring B¹ is selected from the group consisting of

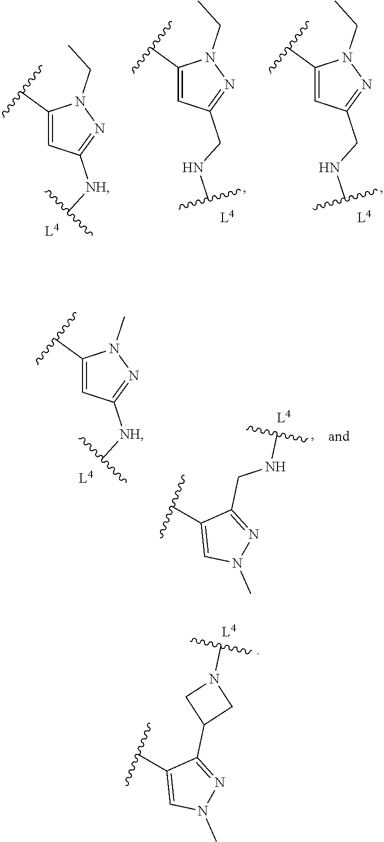

In one embodiment of Formula (CONJ-V) or (CONJ-VA), including any of the foregoing, Ring B¹ is

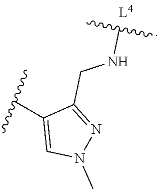 [SMILES: *NCc1nn(C)cc1*]

In one embodiment of Formula (CONJ-V) or (CONJ-VA), including any of the foregoing, Ring B¹ is selected from the group consisting of

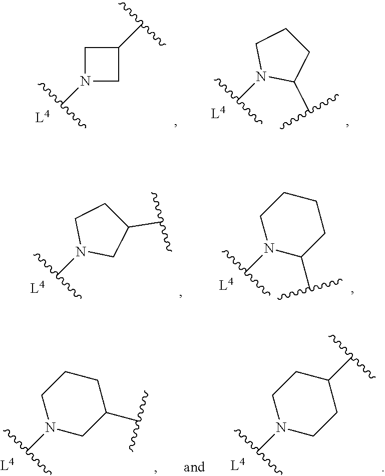 [SMILES: *C1CN(*)C1 ; *C1CCCN1* ; *C1CCN(*)C1 ; *C1CCCCN1* ; *C1CCCN(*)C1 ; *C1CCN(*)CC1]

In one embodiment of Formula (CONJ-V) or (CONJ-VA), including any of the foregoing, Ring B¹ is

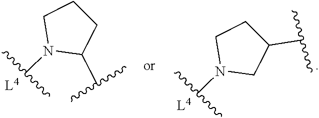 [SMILES: *C1CCCN1* ; *C1CCN(*)C1]

In one embodiment of Formula (CONJ-V) or (CONJ-VA), including any of the foregoing, Ring B¹ is

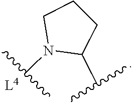 [SMILES: *C1CCCN1*]

In one embodiment of Formula (CONJ-V) or (CONJ-VA), including any of the foregoing, Ring B¹ is selected from the group consisting of

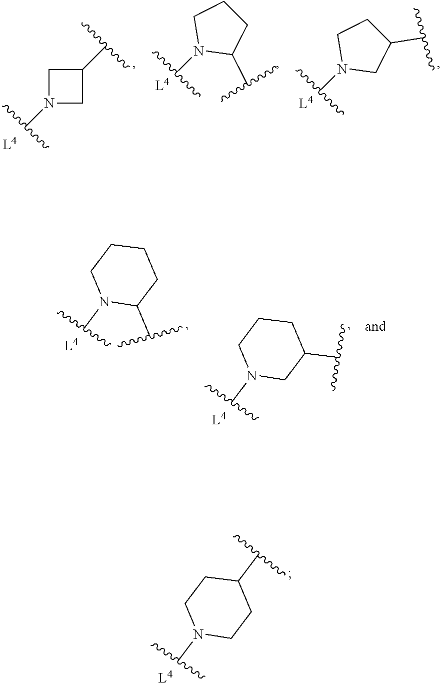 [SMILES: *C1CN(*)C1 ; *C1CCCN1* ; *C1CCN(*)C1 ; *C1CCCCN1* ; *C1CCCN(*)C1 ; *C1CCN(*)CC1]

and Ring A¹ is selected from

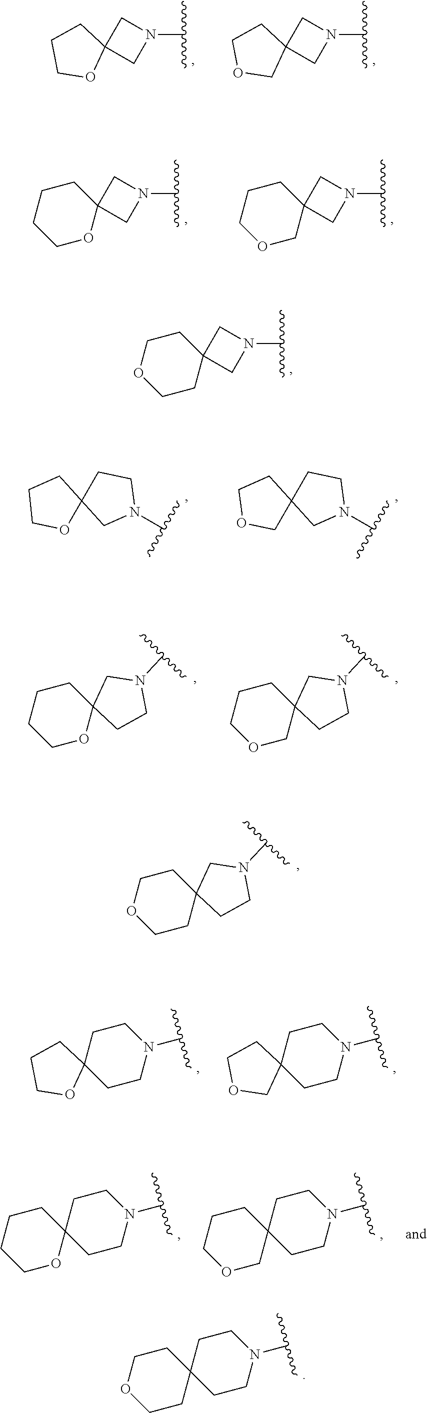

In one embodiment of Formula (CONJ-V) or (CONJ-VA), including any of the foregoing, Ring B¹ is

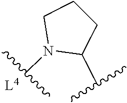 [SMILES: *C1CCCN1*]

### and Ring A¹ is

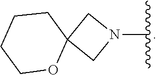 [SMILES: *N1CC2(CCCCO2)C1]

In one embodiment of Formula (CONJ-V) or (CONJ-VA), including any of the foregoing, Ring B¹ is

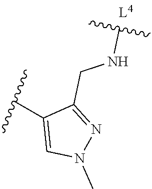 [SMILES: *NCc1nn(C)cc1*]

### and Ring A¹ is

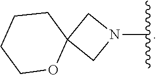 [SMILES: *N1CC2(CCCCO2)C1]

Non-limiting examples of Formula (CONJ-IA) include:

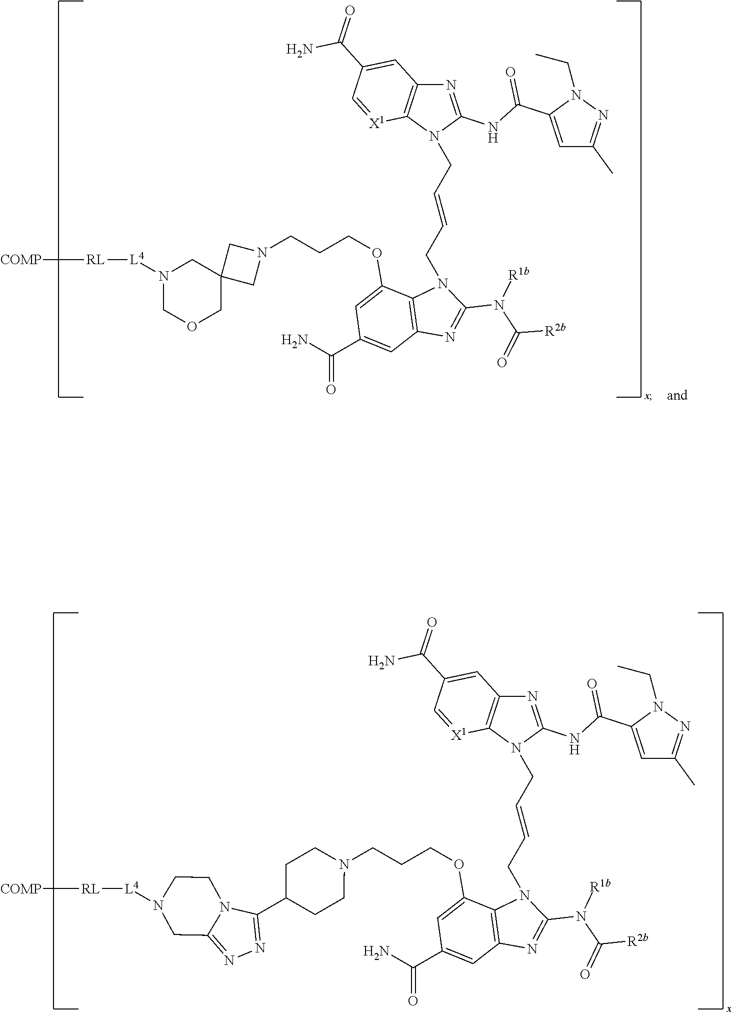

or a stereoisomer thereof.

Non-limiting examples of Formula (CONJ-IB) include:

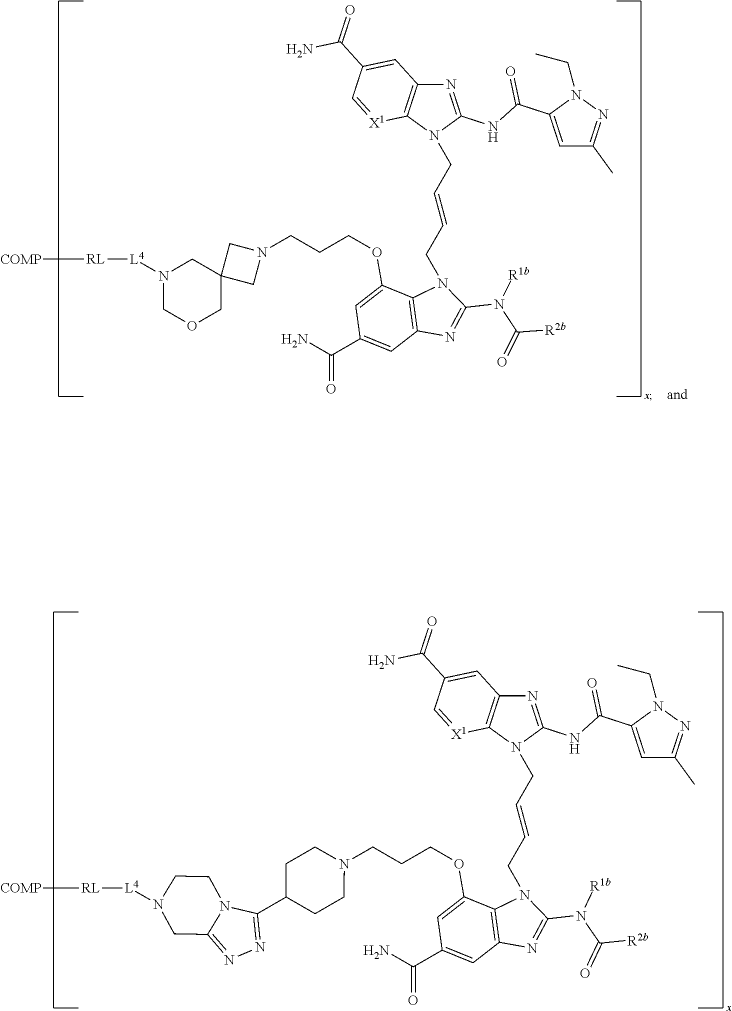

or a stereoisomer thereof.

Non-limiting examples of Formula (CONJ-IIA), (CONJ-IIB), or (CONJ-IIC) include:

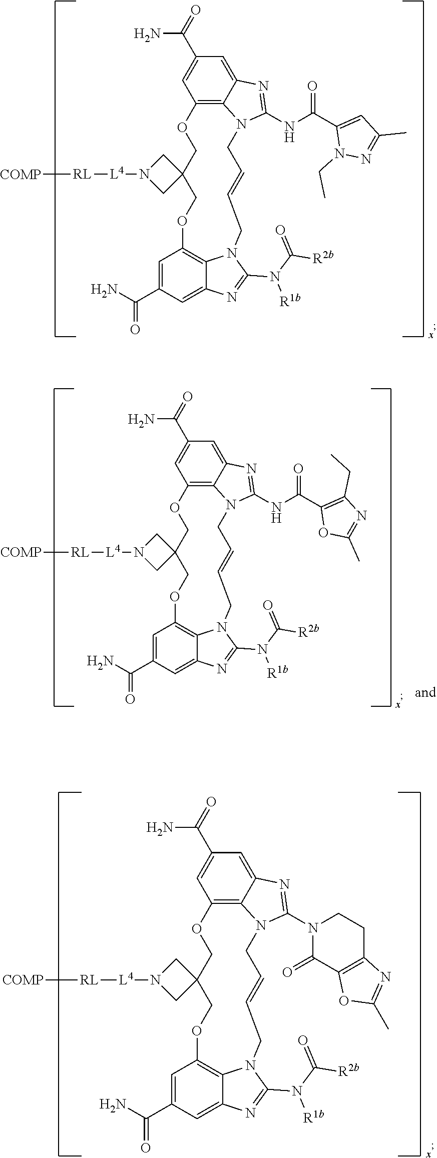

or a stereoisomer thereof.

A non-limiting example of Formula (CONJ-IIIA) includes:

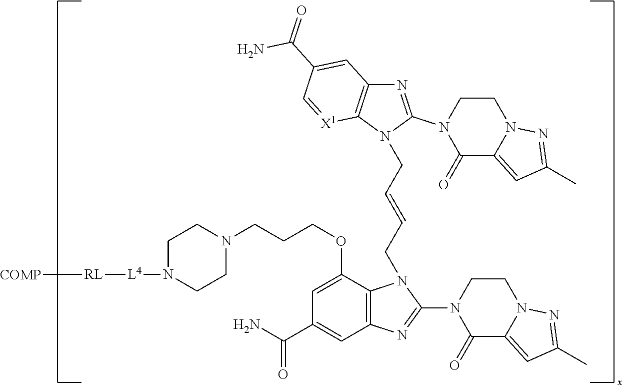

or a stereoisomer thereof.

Non-limiting examples of Formula (CONJ-IVA) include:

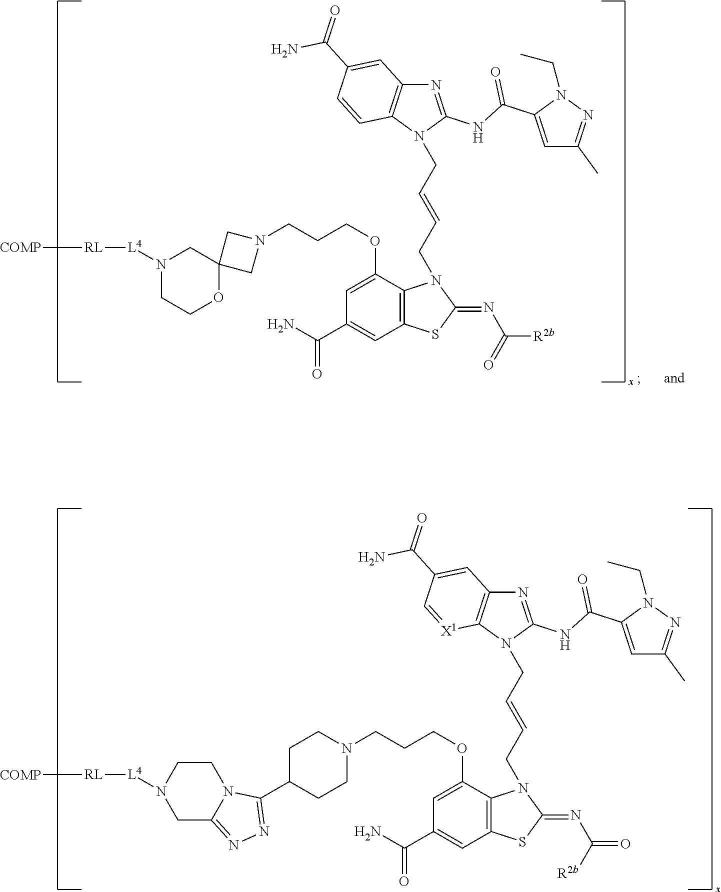

or a stereoisomer thereof.

Non-limiting examples of Formula (CONJ-VA) include:

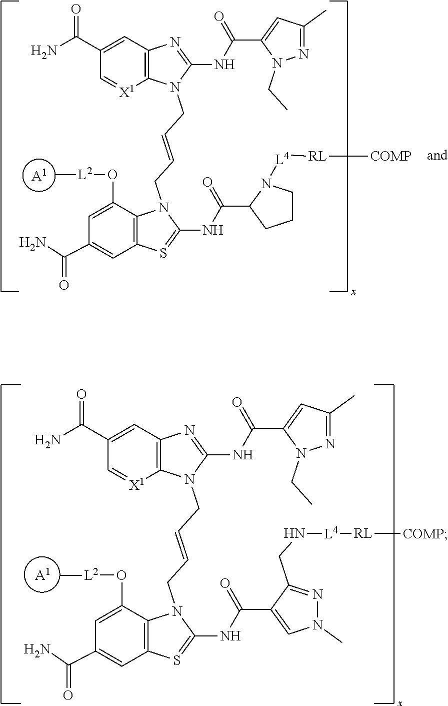

or a stereoisomer thereof.

In one embodiment of Formula (CONJ-IA), L¹ is —CH═CH—; R^{1a} and R^{1b} are both hydrogen; Ring A¹ is

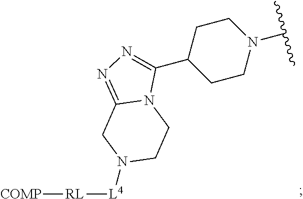

### and R^{2b} is

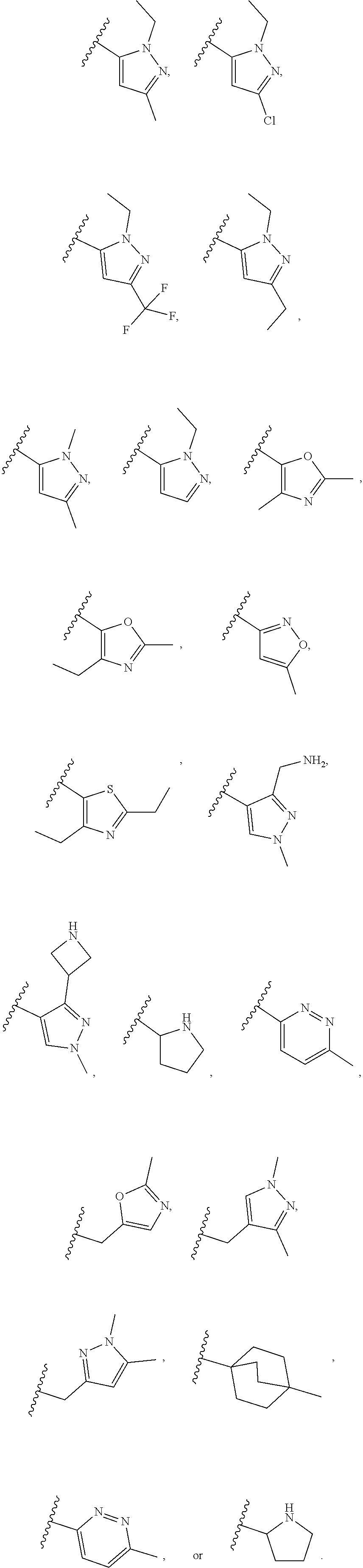

In one embodiment of Formula (CONJ-IA), L¹ is —CH═CH—; R^{1a} and R^{1b} are both hydrogen; Ring A¹ is

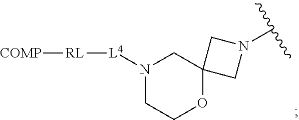

### and R^{2b} is

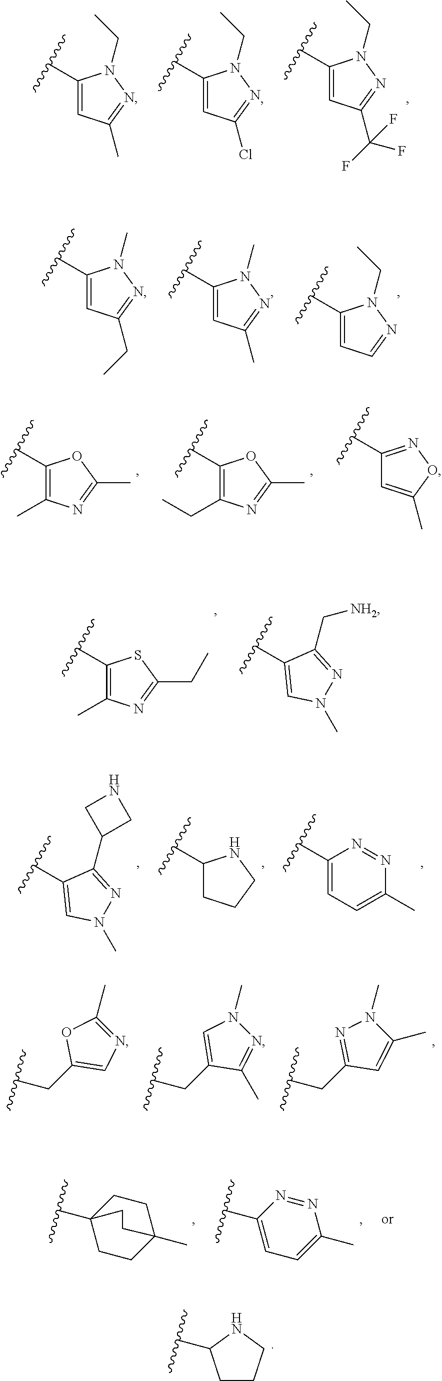

In one embodiment of Formula (CONJ-IA), L¹ is —CH═CH—; R^{1a} is hydrogen; R^{1b} and R^{2b} are joined together to form

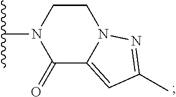 [SMILES: *N1CCn2nc(C)cc2C1=O]

### and Ring A¹ is

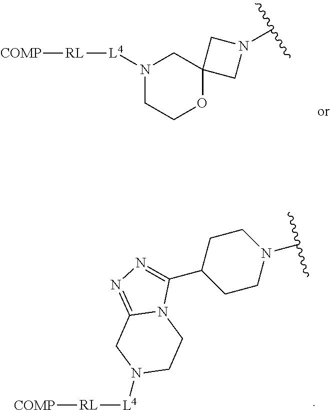

In one embodiment of Formula (CONJ-IB) L¹ is —CH═CH—; R^{1a} is hydrogen; R^{1b} and R^{2b} are joined together to form

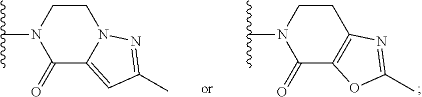 [SMILES: *N1CCn2nc(C)cc2C1=O ; *N1CCc2nc(C)oc2C1=O]

### and Ring A¹ is

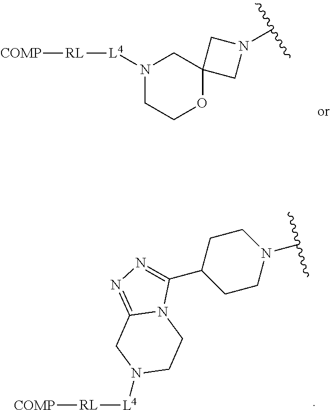

In one embodiment of Formula (CONJ-IIIA), L¹ is —CH═CH—; R^{9b} and R^{10b} are joined together to form

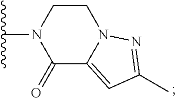 [SMILES: *N1CCn2nc(C)cc2C1=O]

### and Ring A¹ is

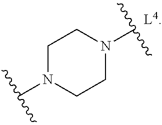 [SMILES: *N1CCN(*)CC1]

In one embodiment of Formula (CONJ-IVA), L¹ is —CH═CH—; R^{1a} is hydrogen; Ring A¹ is

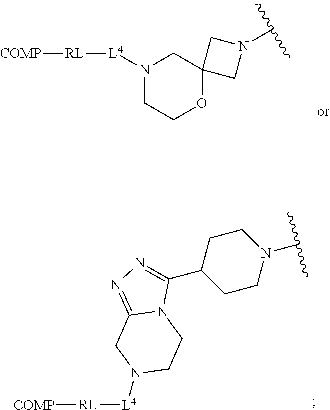

### and R^{2b} is

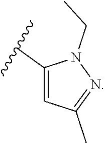 [SMILES: *c1cc(C)nn1CC]

In one embodiment of Formula (CONJ-VA), L¹ is —CH═CH—; R^{1a} and R^{1b} are both hydrogen; Ring A¹ is

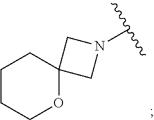 [SMILES: *N1CC2(CCCCO2)C1]

### and Ring B¹ is

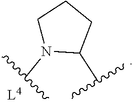 [SMILES: *C1CCCN1*]

In one embodiment of Formula (CONJ-VA), L¹ is —CH═CH—; R^{1a} and R^{1b} are both hydrogen; Ring A¹ is

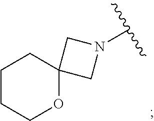 [SMILES: *N1CC2(CCCCO2)C1]

### and Ring B¹ is

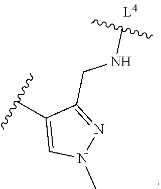 [SMILES: *NCc1nn(C)cc1*]

In one embodiment of Formula (CONJ-I), L¹ is —CH═CH—; R^{1a} and R^{1b} are both hydrogen; R^{2a} is selected from

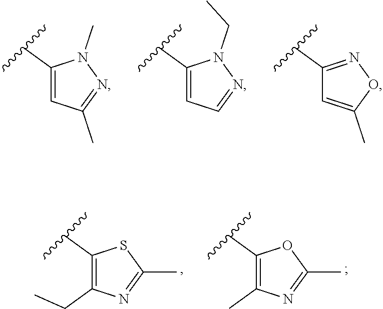

R^{2b} is selected from

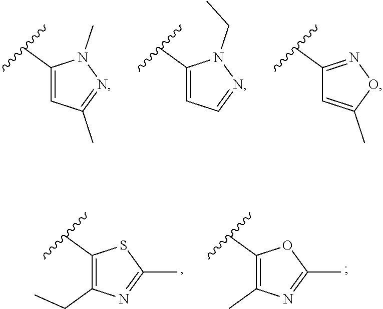

### and Ring A¹ is

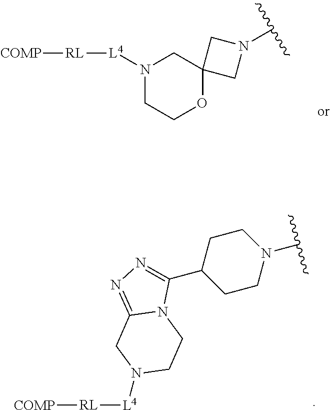

In one embodiment of Formula (CONJ-I), L¹ is —CH₂CH₂—; R^{1a} and R^{1b} are both hydrogen; R^{2a} is

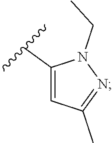 [SMILES: *c1cc(C)nn1CC]

### and R^{2b} is

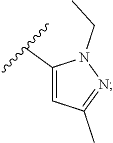 [SMILES: *c1cc(C)nn1CC]

### and Ring A¹ is

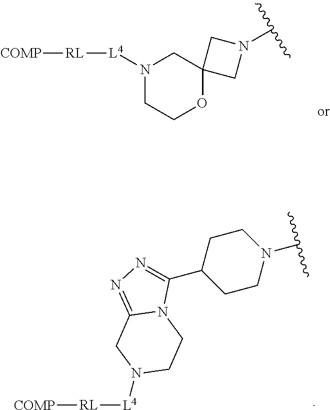

In one embodiment of Formula (CONJ-I)—(CONJ-VA), including any of the foregoing, L⁴ is a linker that comprises a protease cleavable linker, a pH-sensitive linker, or a non-cleavable linker.

In one embodiment of Formula (CONJ-I)—(CONJ-VA), including any of the foregoing, L⁴ is of the formula:

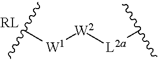

wherein
W¹ and W² are independently absent or a divalent attaching group;
L^{2a} is absent, a protease cleavable linker, or a pH-sensitive linker;

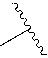

is the point of attachment to the rest of the compound; and

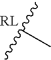

is a bond to RL.

In one embodiment of Formula (CONJ-I)—(CONJ-VA), including any of the foregoing, wherein W¹ is C₁₋₆alkylene-, —C(O)—C₁₋₆alkylene-C(O)—, —C(O)(C₁₋₆alkylene)NR¹⁴C(O)—, —C(O)(C₁₋₆alkylene)OC(O)—, —C(O)(C₁₋₆alkylene)SC(O)—; wherein R¹⁴ is hydrogen or optionally substituted C₁₋₆alkyl, RL is connected to W¹ at —C(O)—, and the C₁₋₆ alkylene is optionally substituted with one, two, or three substituents selected from halogen, alkyl, haloalkyl, hydroxyl, amino, alkylamino, and alkoxy.

In one embodiment of Formula (CONJ-I)—(CONJ-VA) or (LP-1)-(LP-VA), including any of the foregoing, W¹ is absent.

In one embodiment of Formula (CONJ-I)—(CONJ-VA) or (LP-1)-(LP-VA), including any of the foregoing, W² is

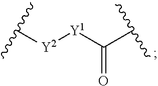

wherein Y¹ is absent or —C₁₋₁₀ alkylene-;

Y² is absent, a divalent water-soluble polymer, —NR¹⁴—C₁₋₁₀ alkylene-, —NR¹⁴—C(O)—C₁₋₁₀alkylene-, or —O—C(O)—(C₁₋₁₀alkylene)-;

R¹⁴ is hydrogen or C₁₋₆ alkyl; and

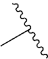

is the point of attachment to the rest of the compound and the carbonyl is attached to L^{2a}.

wherein the C₁₋₁₀alkylene of Y¹ or Y² is optionally substituted with one, two, or three substituents selected from a halogen, alkyl, haloalkyl, hydroxyl, amino, alkylamino, and alkoxy.

In one embodiment of Formula (CONJ-I)—(CONJ-VA) or (LP-1)-(LP-VA), including any of the foregoing, Y¹ is —CH₂—, —(CH2)₂—, or —(CH2)₅—.

In one embodiment of Formula (CONJ-I)—(CONJ-VA) or (LP-1)-(LP-VA), including any of the foregoing, Y² is absent. In one embodiment of Formula (CONJ-I)—(CONJ-VA) or (LP-1)-(LP-VA), including any of the foregoing, Y² is —NR¹⁴—C(O)—C₁₋₁₀ alkylene-. In one embodiment of Formula (CONJ-I)—(CONJ-VA) or (LP-1)-(LP-VA), including any of the foregoing, Y² is a divalent water-soluble polymer.

In one embodiment of Formula (CONJ-I)—(CONJ-VA) or (LP-1)-(LP-VA), including any of the foregoing, Y¹ is —CH₂—, —(CH2)₂—, or —(CH2)₅— and Y² is absent.

In one embodiment of Formula (CONJ-I)—(CONJ-VA) or (LP-1)-(LP-VA), including any of the foregoing, Y¹ is —(CH₂)₂— and Y² is

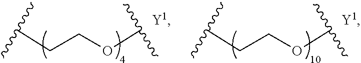

or —NR¹⁴—C(O)—C₅ alkylene- wherein the —NR¹⁴—is attached to Y¹ wherein

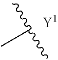

is the point of attachment to Y¹.

In one embodiment of Formula (CONJ-I)—(CONJ-VA) or (LP-1)-(LP-VA), including any of the foregoing, the divalent water-soluble polymer of W² is

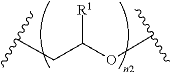

wherein R¹ is hydrogen or methyl and n2 is an integer between 1 and 50, inclusive and

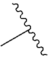

is the point of attachment to the rest of the compound. In one embodiment of Formula (CONJ-I)—(CONJ-VA) or (LP-1)-(LP-VA), including any of the foregoing, n2 is an integer between 1 and 20. In one embodiment of Formula (CONJ-I)—(CONJ-VA) or (LP-1)-(LP-VA), including any of the foregoing, n2 is an integer between 1 and 40. In one embodiment of Formula (CONJ-I)—(CONJ-VA) or (LP-1)-(LP-VA), including any of the foregoing, n2 is an integer between 1 and 30. In one embodiment of Formula (CONJ-I)—(CONJ-VA) or (LP-1)-(LP-VA), including any of the foregoing, n2 is an integer between 1 and 25. In one embodiment of Formula (CONJ-I)—(CONJ-VA) or (LP-1)-(LP-VA), including any of the foregoing, n2 is an integer between 1 and 20. In one embodiment of Formula (CONJ-I)—(CONJ-VA) or (LP-1)-(LP-VA), including any of the foregoing, n2 is an integer between 1 and 15. In one embodiment of Formula (CONJ-I)—(CONJ-VA) or (LP-1)-(LP-VA), including any of the foregoing, n2 is an integer between 1 and 10. In one embodiment of Formula (CONJ-I)—(CONJ-VA) or (LP-1)-(LP-VA), including any of the foregoing, n2 is an integer between 1 and 5. In one embodiment of Formula (CONJ-I)—(CONJ-VA) or (LP-1)-(LP-VA), including any of the foregoing, R¹ is hydrogen. In one embodiment of Formula (CONJ-I)—(CONJ-VA) or (LP-1)-(LP-VA), including any of the foregoing, R¹ is methyl.

In one embodiment of Formula (CONJ-I)—(CONJ-VA) or (LP-1)-(LP-VA), including any of the foregoing, W² is

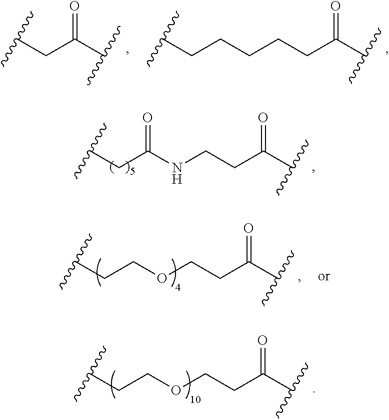

In one embodiment of Formula (CONJ-I)—(CONJ-VA) or (LP-1)-(LP-VA), including any of the foregoing, W² is

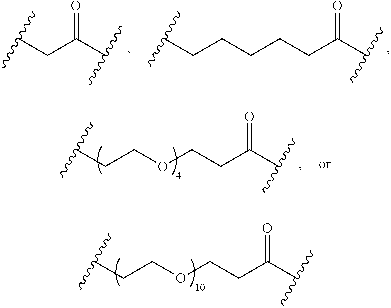

and W¹ is absent. In one embodiment of Formula (CONJ-I)—(CONJ-VA) or (LP-1)-(LP-VA), including any of the foregoing, W² is

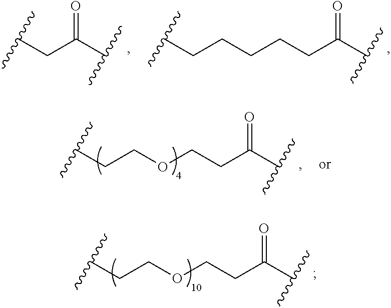

W¹ is absent; and L^{2a} is absent.

In one embodiment of Formula (CONJ-I)—(CONJ-VA) or (LP-1)-(LP-VA), including any of the foregoing, L^{2a} comprises —C(O)C₁₋₆alkylNR¹⁴— wherein —NR¹⁴—is attached to W². In one embodiment of Formula (CONJ-I)—(CONJ-VA) or (LP-1)-(LP-VA), including any of the foregoing, L^{2a} comprises —C(O)C₁₋₆alkylN(CH₃)—. In one embodiment of Formula (CONJ-I)—(CONJ-VA) or (LP-1)-(LP-VA), including any of the foregoing, L^{2a} comprises —C(O)CH₂alkylN(CH₃)—.

In one embodiment of Formula (CONJ-I)—(CONJ-VA) or (LP-1)-(LP-VA), including any of the foregoing, L^{2a} comprises —(C(O)CHR^{6a}NR^{7a})ₐ—wherein R^{6a} is an amino acid sidechain residue; R^{7a} is hydrogen or C₁₋₆ alkyl; and a is an integer between 1 and 10, inclusive. In one embodiment of Formula (CONJ-I)—(CONJ-VA) or (LP-1)-(LP-VA), including any of the foregoing, L^{2a} comprises —(C(O)CHR^{6a}NR^{7a})₂—. In one embodiment of Formula (CONJ-I)—(CONJ-VA) or (LP-1)-(LP-VA), including any of the foregoing, L²a comprises —(C(O)CHR^{6a}NR^{7a})₄—. In one embodiment of Formula (CONJ-I)—(CONJ-VA) or (LP-1)-(LP-VA), including any of the foregoing, R^{6a} is an amino acid sidechain independently selected from Phe, Lys, Val, Ala, Asn, Cit, Phe, Leu, Ile, Arg, and Trp. In one embodiment of Formula (CONJ-I)—(CONJ-VA) or (LP-1)-(LP-VA), including any of the foregoing, R^{6a} is an amino acid sidechain independently selected from Val, Arg, Ala, and Asn.

In one embodiment of Formula (CONJ-I)—(CONJ-VA) or (LP-1)-(LP-VA), including any of the foregoing, L^{2a} comprises

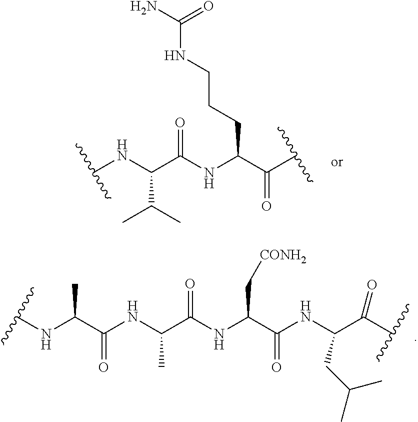 [SMILES: *N[C@H](C(=O)N[C@@H](CCCNC(N)=O)C(*)=O)C(C)C ; *N[C@@H](C)C(=O)N[C@@H](C)C(=O)N[C@@H](CC(N)=O)C(=O)N[C@@H](CC(C)C)C(*)=O]

In one embodiment of Formula CONJ-I)—(CONJ-VA) or (LP-1)-(LP-VA), including any of the foregoing, L^{2a} comprises

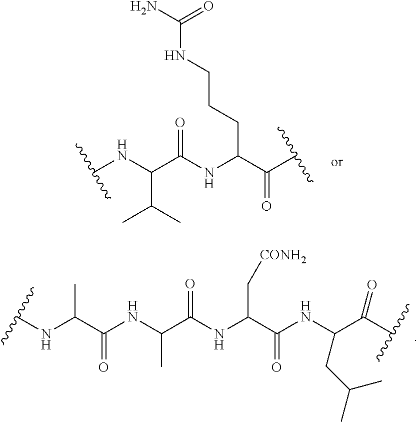 [SMILES: *NC(C(=O)NC(CCCNC(N)=O)C(*)=O)C(C)C ; *NC(C)C(=O)NC(C)C(=O)NC(CC(N)=O)C(=O)NC(CC(C)C)C(*)=O]

In one embodiment of Formula (CONJ-I)—(CONJ-VA) or (LP-1)-(LP-VA), including any of the foregoing, L^{2a} comprises —PABC—(C(O)CHR^{6a}NR^{7a})ₐ—wherein PABC is

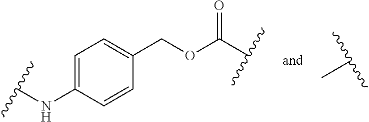

is the point of attachment to the rest of the compound. In one embodiment of Formula (CONJ-I)—(CONJ-VA) or (LP-1)-(LP-VA), including any of the foregoing, L^{2a} comprises —PABC—(C(O)CHR^{6a}NR^{7a})₂—. In one embodiment of Formula (CONJ-I)—(CONJ-VA) or (LP-1)-(LP-VA), including any of the foregoing, L^{2a} comprises —PABC—(C(O)CHR^{6a}NR^{7a})₄—.

In one embodiment of Formula (CONJ-I)—(CONJ-VA) or (LP-1)-(LP-VA), including any of the foregoing, L^{2a} comprises

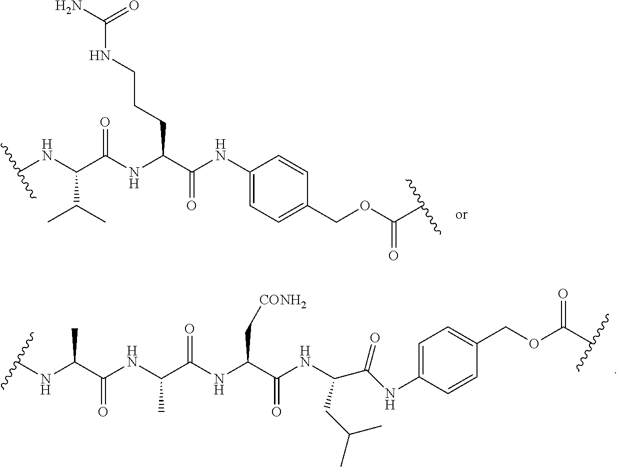 [SMILES: *N[C@H](C(=O)N[C@@H](CCCNC(N)=O)C(=O)Nc1ccc(COC(*)=O)cc1)C(C)C ; *N[C@@H](C)C(=O)N[C@@H](C)C(=O)N[C@@H](CC(N)=O)C(=O)N[C@@H](CC(C)C)C(=O)Nc1ccc(COC(*)=O)cc1]

In one embodiment of Formula (CONJ-I)—(CONJ-VA) or (LP-1)-(LP-VA), including any of the foregoing, L^{2a} comprises

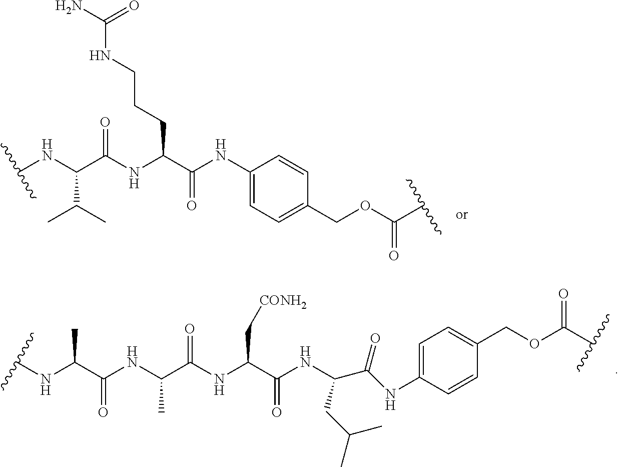 [SMILES: *N[C@H](C(=O)N[C@@H](CCCNC(N)=O)C(=O)Nc1ccc(COC(*)=O)cc1)C(C)C ; *N[C@@H](C)C(=O)N[C@@H](C)C(=O)N[C@@H](CC(N)=O)C(=O)N[C@@H](CC(C)C)C(=O)Nc1ccc(COC(*)=O)cc1]

In one embodiment of Formula (CONJ-I)—(CONJ-VA) or (LP-1)-(LP-VA), including any of the foregoing, L^{2a} comprises —C(O)C₁₋₆alkylNR¹⁴—PABC—(C(O)CHR^{6a}N^{7a})ₐ—. In one embodiment of Formula (CONJ-I)—(CONJ-VA) or (LP-1)-(LP-VA), including any of the foregoing, L^{2a} comprises —C(O)C₁₋₆alkylN(CH₃)—PABC—(C(O)CHR^{6a}N^{7a})ₐ—. In one embodiment of Formula (CONJ-I)—(CONJ-VA) or (LP-1)-(LP-VA), including any of the foregoing, L^{2a} comprises

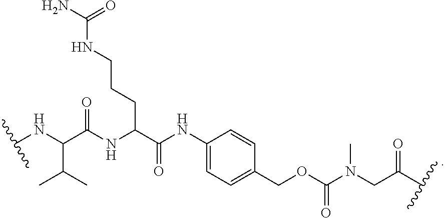 [SMILES: *NC(C(=O)NC(CCCNC(N)=O)C(=O)Nc1ccc(COC(=O)N(C)CC(*)=O)cc1)C(C)C]

In one embodiment of Formula (CONJ-I)—(CONJ-VA) or (LP-1)-(LP-VA), including any of the foregoing, L^{2a} comprises —(C(O)CHR^{6a}NR^{7a})ₐ—PABC—(C(O)CHR^{6a}NR^{7a})ₐ—. In one embodiment of Formula (CONJ-I)—(CONJ-VA) or (LP-1)-(LP-VA), including any of the foregoing, L^{2a} comprises —C(O)CHR^{6a}NR^{7a}—PABC—(C(O)CHR^{6a}NR^{7a})ₐ—. In one embodiment of Formula (CONJ-I)—(CONJ-VA) or (LP-1)-(LP-VA), including any of the foregoing, L^{2a} comprises —C(O)CHR^{6a}NH—PABC—(C(O)CHR^{6a}NH)ₐ—. In one embodiment of Formula (CONJ-I)—(CONJ-VA) or (LP-1)-(LP-VA), including any of the foregoing, L^{2a} comprises

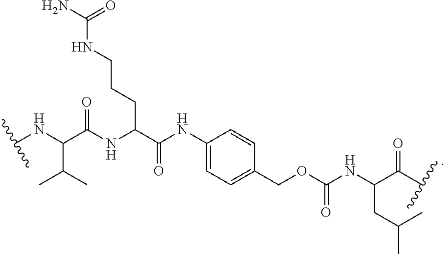 [SMILES: *NC(C(=O)NC(CCCNC(N)=O)C(=O)Nc1ccc(COC(=O)NC(CC(C)C)C(*)=O)cc1)C(C)C]

In one embodiment of Formula (CONJ-I)—(CONJ-VA) or (LP-1)-(LP-VA), including any of the foregoing, L^{2a} comprises

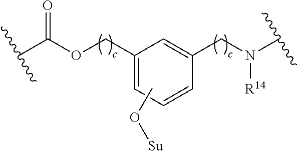

wherein Su is a hexose form of a monosaccharide; c is an integer independently selected from 1, 2, and 3; and —NR¹⁴— is attached to W². In one embodiment of Formula (CONJ-I)—(CONJ-VA) or (LP-1)-(LP-VA), including any of the foregoing, L^{2a} comprises

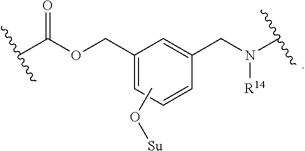

In one embodiment of Formula (CONJ-I)—(CONJ-VA) or (LP-1)-(LP-VA), including any of the foregoing, L^{2a} comprises

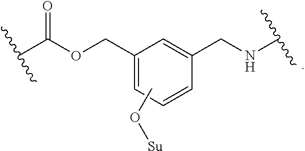

In one embodiment of Formula (CONJ-I)—(CONJ-VA) or (LP-1)-(LP-VA), including any of the foregoing, L^{2a} comprises

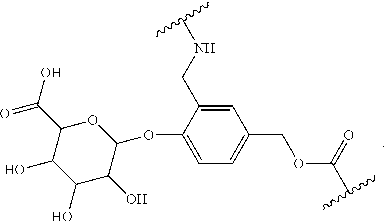 [SMILES: *NCc1cc(COC(*)=O)ccc1OC1OC(C(=O)O)C(O)C(O)C1O]

In one embodiment of Formula (CONJ-I)—(CONJ-VA) or (LP-1)-(LP-VA), including any of the foregoing, L^{2a} comprises

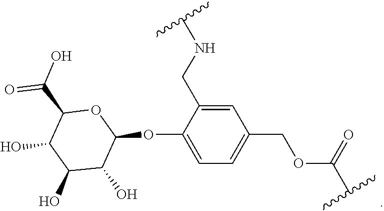 [SMILES: *NCc1cc(COC(*)=O)ccc1O[C@@H]1O[C@H](C(=O)O)[C@@H](O)[C@H](O)[C@H]1O]

In one embodiment of Formula (CONJ-I)—(CONJ-VA) or (LP-1)-(LP-VA), including any of the foregoing, L^{2a} comprises

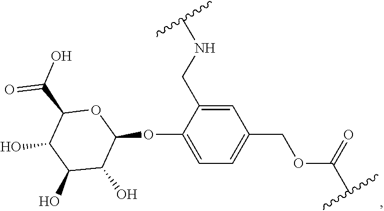 [SMILES: *NCc1cc(COC(*)=O)ccc1O[C@@H]1O[C@H](C(=O)O)[C@@H](O)[C@H](O)[C@H]1O]

Y¹ is —(CH₂)₂— and Y² is —NR¹⁴—C(O)—C₁₋₁₀ alkylene- wherein the —NR¹⁴—is attached to Y¹.

In one embodiment of Formula (CONJ-I)—(CONJ-VA) or (LP-1)-(LP-VA), including any of the foregoing, L⁴ is —C(O)—C₁₋₁₀ alkylene-. In one embodiment of Formula (CONJ-I)—(CONJ-VA) or (LP-1)-(LP-VA), including any of the foregoing, L⁴ is —C(O)—C₁₋₅ alkylene-.

In one embodiment of Formula (CONJ-I)—(CONJ-VA) or (LP-1)-(LP-VA), including any of the foregoing, L⁴ is —PABC—(C(O)CHR^{6a}NR^{7a})ₐ—C(O)—C₁₋₁₀ alkylene-(CH₂CH(R¹)O)ₙ₂—. In one embodiment of Formula (CONJ-I)—(CONJ-VA) or (LP-1)-(LP-VA), including any of the foregoing, L⁴ is —PABC—(C(O)CHR^{6a}NR^{7a})₂—C(O)—C₂ alkylene-(CH₂CH(R¹)O)₄—. In one embodiment of Formula (CONJ-I)—(CONJ-VA) or (LP-1)-(LP-VA), including any of the foregoing, L⁴ is —PABC—(C(O)CHR^{6a}NR^{7a})₂—C(O)—C₂ alkylene-(CH₂CH(R¹)O)₁₀—. In one embodiment of Formula (CONJ-I)—(CONJ-VA) or (LP-1)-(LP-VA), including any of the foregoing, L⁴ is —PABC—(C(O)CHR^{6a}NR^{7a})₄—C(O)—C₂ alkylene-(CH₂CH(R¹)O)₄—. In one embodiment of Formula (CONJ-I)—(CONJ-VA) or (LP-1)-(LP-VA), including any of the foregoing, L⁴ is —PABC—(C(O)CHR^{6a}NR^{7a})₄—C(O)—C₂ alkylene-(CH₂CH(R¹)O)₁₀—.

In one embodiment of Formula (CONJ-I)—(CONJ-VA) or (LP-1)-(LP-VA), including any of the foregoing, L⁴ is —(C(O)CHR^{6a}NR^{7a})ₐ—C(O)—C₁₋₁₀ alkylene-. In one embodiment of Formula (CONJ-I)—(CONJ-VA) or (LP-1)-(LP-VA), including any of the foregoing, L⁴ is —(C(O)CHR^{6a}NR^{7a})ₐ—C(O)—C₅ alkylene-. In one embodiment of Formula (CONJ-I)—(CONJ-VA) or (LP-1)-(LP-VA), including any of the foregoing, L⁴ is —C(O)CHR^{6a}NR^{7a}—C(O)—C₁₋₁₀ alkylene-. In one embodiment of Formula (CONJ-I)—(CONJ-VA) or (LP-1)-(LP-VA), including any of the foregoing, L⁴ is —C(O)CHR^{6a}NR^{7a}—C(O)—C₅ alkylene-.

In one embodiment of Formula (CONJ-I)—(CONJ-VA) or (LP-1)-(LP-VA), including any of the foregoing, L⁴ is —C(O)—C₁₋₁₀ alkylene-(CH₂CH(R¹)O)ₙ₂—. In one embodiment of Formula (CONJ-I)—(CONJ-VA) or (LP-1)-(LP-VA), including any of the foregoing, L⁴ is —C(O)—C₂ alkylene-(CH₂CH(R¹)O)ₙ₂—. In one embodiment of Formula (CONJ-I)—(CONJ-VA) or (LP-1)-(LP-VA), including any of the foregoing, L⁴ is —C(O)—C₁₋₁₀ alkylene-(CH₂CH(R¹)O)₄—. In one embodiment of Formula (CONJ-I)—(CONJ-VA) or (LP-1)-(LP-VA), including any of the foregoing, L⁴ is —C(O)—C₁₋₁₀ alkylene-(CH₂CH(R₁)O)₁₀—. In one embodiment of Formula (CONJ-I)—(CONJ-VA) or (LP-1)-(LP-VA), including any of the foregoing, L⁴ is —C(O)—C₂ alkylene-(CH₂CH(R¹)O)₄—. In one embodiment of Formula (CONJ-I)—(CONJ-VA) or (LP-1)-(LP-VA), including any of the foregoing, L⁴ is —C(O)—C₂ alkylene-(CH₂CH(R¹)O)₁₀—. In one embodiment of Formula (CONJ-I)—(CONJ-VA) or (LP-1)-(LP-VA), including any of the foregoing, L⁴ is —C(O)CH₂N(CH₃)C(O)C₁₋₆alkyl-.

In one embodiment of Formula (CONJ-I)—(CONJ-VA) or (LP-1)-(LP-VA), including any of the foregoing, L^{2a} is

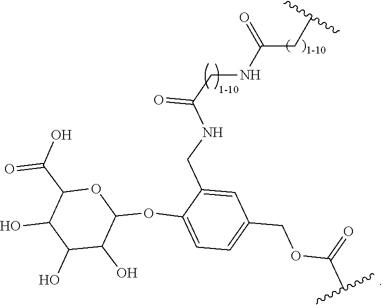

In one embodiment of Formula (CONJ-I)—(CONJ-VA) or (LP-1)-(LP-VA), including any of the foregoing, L^{2a} is

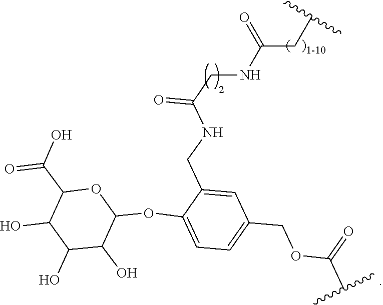

In one embodiment of Formula (CONJ-I)—(CONJ-VA) or (LP-1)-(LP-VA), including any of the foregoing, L^{2a} is

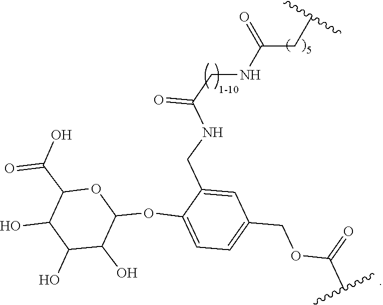

In one embodiment of Formula (CONJ-I)—(CONJ-VA) or (LP-1)-(LP-VA), including any of the foregoing, L⁴ is selected from:

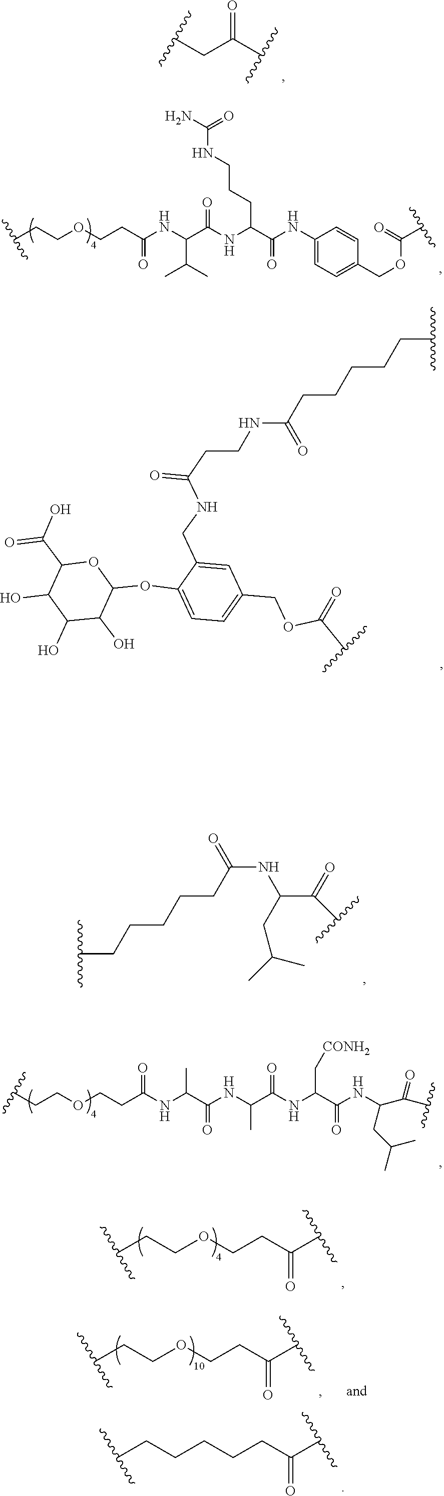

In one embodiment of Formula (CONJ-I)—(CONJ-VA) or (LP-1)-(LP-VA), including any of the foregoing, L⁴ is selected from:

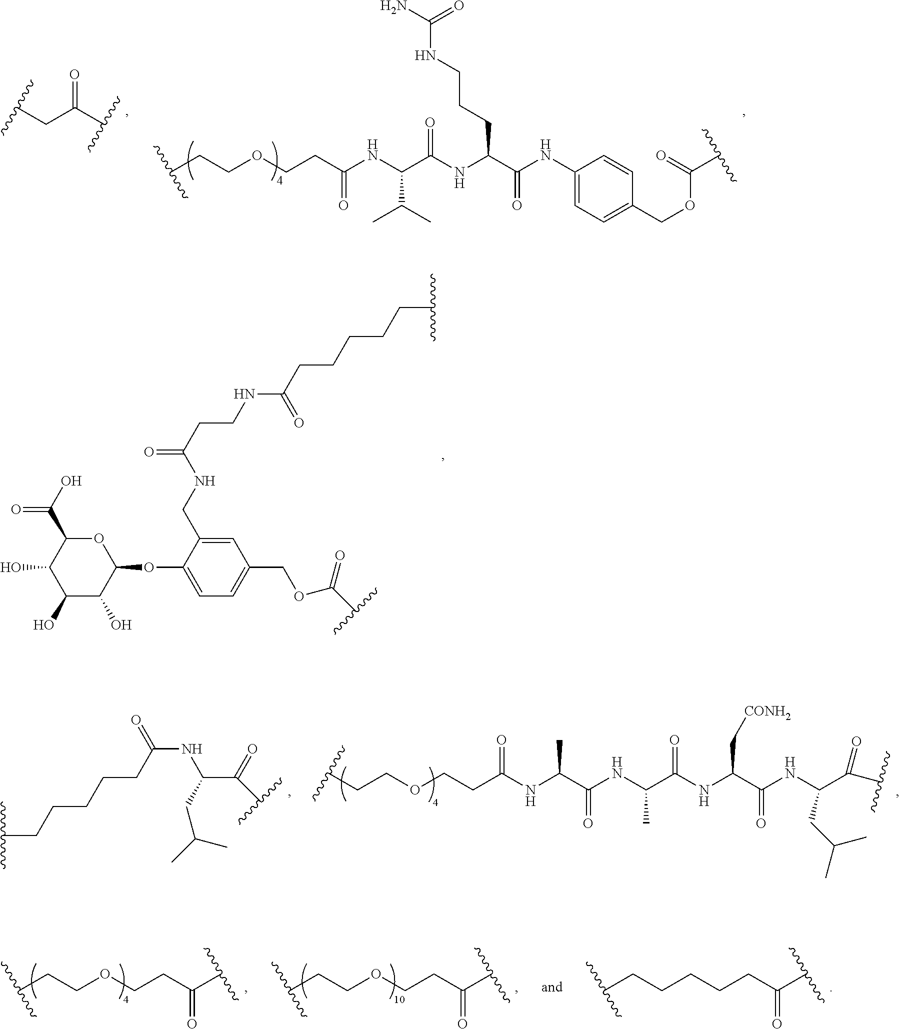

In one embodiment of Formula (CONJ-I)—(CONJ-VA) or (LP-1)-(LP-VA), including any of the foregoing, L⁴ is selected from

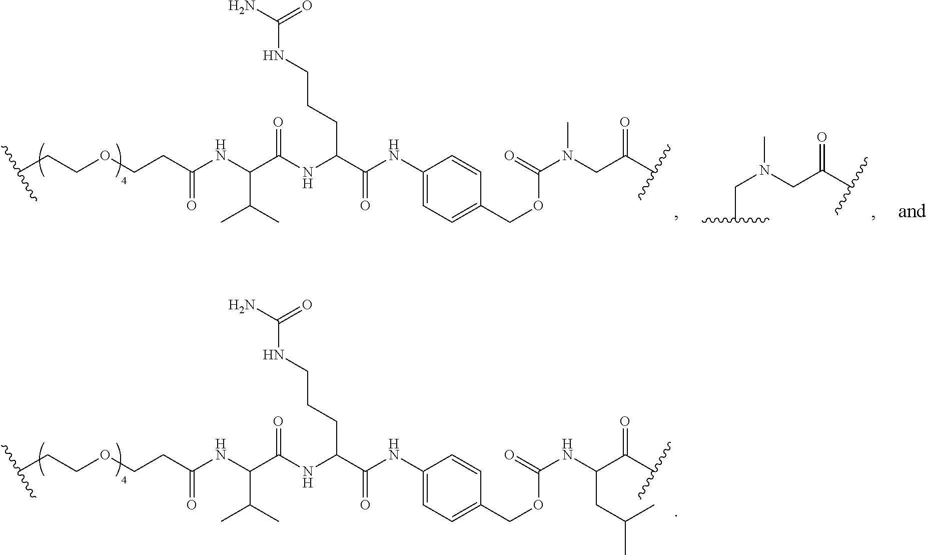

In one embodiment of Formula (CONJ-I)—(CONJ-VA) or (LP-1)-(LP-VA), including any of the foregoing, L⁴ is selected from

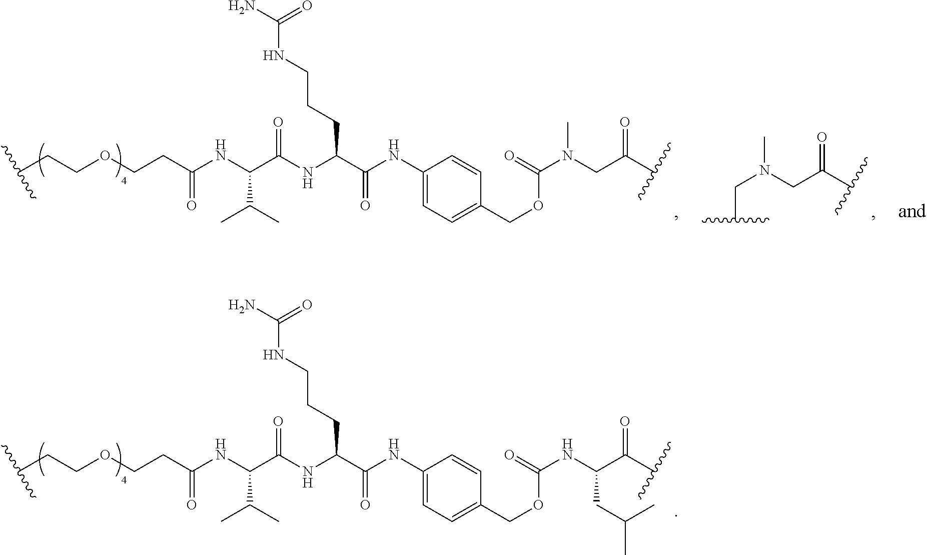

In one embodiment of Formula (CONJ-I)—(CONJ-VA)), RL is a group comprising a triazole, pyridazine, a thiol, or an oxime. In one embodiment of Formula (CONJ-I)—(CONJ-VA)), RL is a group comprising a triazole. In one embodiment of Formula (CONJ-I)—(CONJ-VA)), RL is a group comprising a pyridazine. In one embodiment of Formula (CONJ-I)—(CONJ-VA)), RL is a group comprising a thiol. In one embodiment of Formula (CONJ-I)—(CONJ-VA)), RL is a group comprising an oxime.

In one embodiment of Formula (CONJ-I)—(CONJ-VA)), RL is selected from the group consisting of

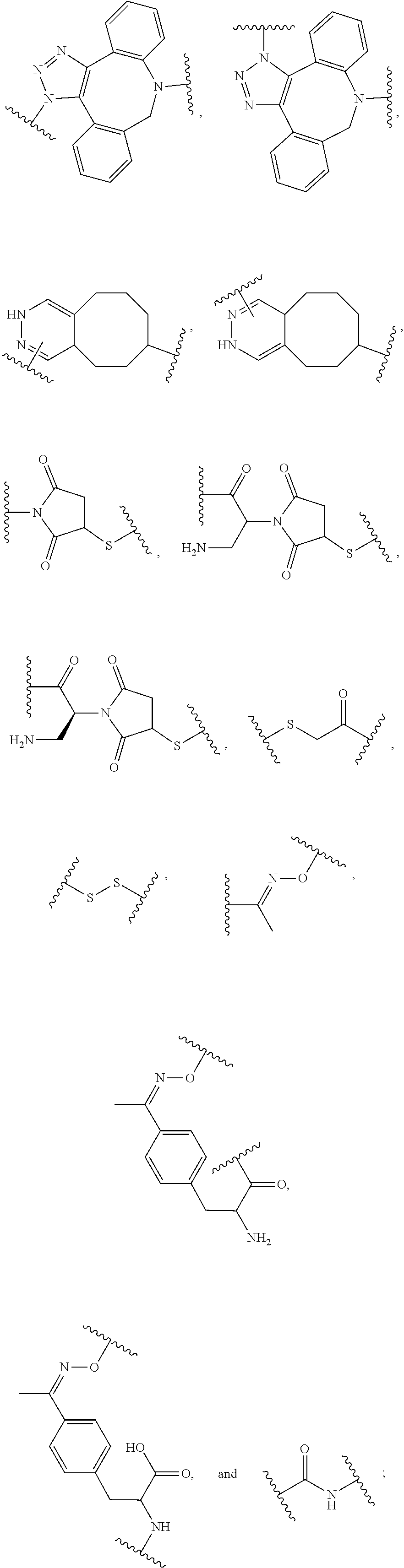

wherein

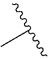

each is a point of attachment to the rest of the compound.
In one embodiment of Formula (CONJ-I)—(CONJ-VA)), RL is

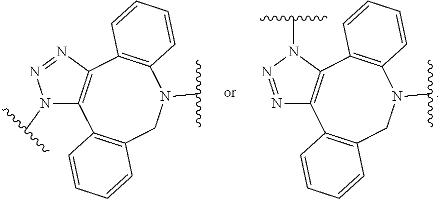 [SMILES: *N1Cc2ccccc2-c2c(nnn2*)-c2ccccc21 ; *N1Cc2ccccc2-c2nnn(*)c2-c2ccccc21]

In one embodiment of Formula (CONJ-I)—(CONJ-VA)), RL is

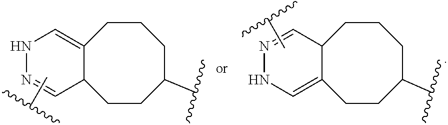

In one embodiment of Formula (CONJ-I)—(CONJ-VA)) RL is

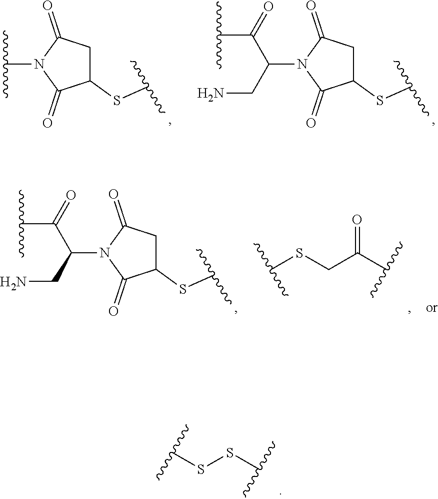

In one embodiment of Formula (CONJ-I)—(CONJ-VA)), RL is

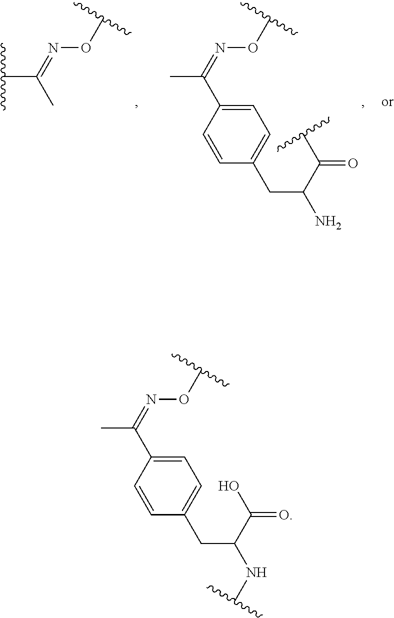

In one embodiment of Formula (CONJ-I)—(CONJ-VA), including any of the foregoing, RL is

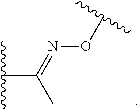 [SMILES: *O/N=C(/*)C]

In one embodiment of Formula (CONJ-I)—(CONJ-VA), including any of the foregoing, RL is

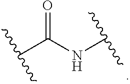 [SMILES: *NC(*)=O]

The COMP of Formula (CONJ-I)—(CONJ-VA) can be any macromolecule deemed suitable by the person of skill in the art. In certain embodiments, the macromolecule is a second compound. In certain embodiments, the macromolecule is a protein, peptide, antibody or antigen-binding fragment thereof, nucleic acid, carbohydrate, or other large molecule composed of polymerized monomers. In certain embodiments, the macromolecule is a peptide of two or more residues. In certain embodiments, the macromolecule is a peptide of ten or more residues. In certain embodiments, the macromolecule is at least 1000 Da in mass. In certain embodiments, the macromolecule comprises at least 1000 atoms. Useful macromolecules are described in the sections below.

In certain embodiments, the macromolecule is a protein, peptide, antibody or antigen binding fragment thereof, nucleic acid, carbohydrate, or other large molecule composed of polymerized monomers. In certain embodiments, the macromolecule is a protein. In certain embodiments, the macromolecule is an antibody, or an antigen binding fragment thereof. In some embodiments, COMP is a polypeptide. In some embodiments, COMP is an antibody. In some embodiments, COMP is an antibody fragment.

In some embodiments, the macromolecule is a known antibody. Useful antibodies include, but are not limited to, rituximab (Rituxan®, IDEC/Genentech/Roche) (see, e.g., U.S. Pat. No. 5,736,137), a chimeric anti-CD20 antibody approved to treat Non-Hodgkin's lymphoma; HuMax-CD20, an anti-CD20 currently being developed by Genmab, an anti-CD20 antibody described in U.S. Pat. No. 5,500,362, AME-133 (Applied Molecular Evolution), hA20 (Immunomedics, Inc.), HumaLYM (Intracel), and PR070769 (PCT Application No. PCT/US2003/040426), trastuzumab (Herceptin®, Genentech) (see, e.g., U.S. Pat. No. 5,677,171), a humanized anti-Her2/neu antibody approved to treat breast cancer; pertuzumab (rhuMab-2C4, Omnitarg®), currently being developed by Genentech; an anti-Her2 antibody (U.S. Pat. No. 4,753,894; cetuximab (Erbitux®, Imclone) (U.S. Pat. No. 4,943,533; PCT Publication No. WO 96/40210), a chimeric anti-EGFR antibody in clinical trials for a variety of cancers; ABX-EGF (U.S. Pat. No. 6,235,883), currently being developed by Abgenix-Immunex-Amgen; HuMax-EGFr (U.S. Pat. No. 7,247,301), currently being developed by Genmab; 425, EMD55900, EMD62000, and EMD72000 (Merck KGaA) (U.S. Pat. No. 5,558,864; Murthy, et al. (1987) Arch. Biochem. Biophys. 252(2): 549-60; Rodeck, et al. (1987) J. Cell. Biochem. 35(4): 315-20; Kettleborough, et al. (1991) Protein Eng. 4(7): 773-83); ICR62 (Institute of Cancer Research) (PCT Publication No. WO 95/20045; Modjtahedi, et al. (1993) J. Cell. Biophys. 22 (I-3): 129-46; Modjtahedi, et al. (1993) Br. J. Cancer 67(2): 247-53; Modjtahedi, et al. (1996) Br. J. Cancer 73(2): 228-35; Modjtahedi, et al. (2003) Int. J. Cancer 105(2): 273-80); TheraCIM hR3 (YM Biosciences, Canada and Centro de Immunologia Molecular, Cuba (U.S. Pat. Nos. 5,891,996; 6,506,883; Mateo, et al. (1997) Immunotechnol. 3(1): 71-81); mAb-806 (Ludwig Institute for Cancer Research, Memorial Sloan-Kettering) (Jungbluth, et al. (2003) Proc. Natl. Acad. Sci. USA. 100(2): 639-44); KSB-102 (KS Biomedix); MR1-1 (IVAX, National Cancer Institute) (PCT Publication No. WO 01/62931A2); and SC100 (Scancell) (PCT Publication No. WO 01/88138); alemtuzumab (Campath®, Millenium), a humanized mAb currently approved for treatment of B-cell chronic lymphocytic leukemia; muromonab-CD3 (Orthoclone OKT3@), an anti-CD3 antibody developed by Ortho Biotech/Johnson & Johnson, ibritumomab tiuxetan (Zevalin®), an anti-CD20 antibody developed by IDEC/Schering AG, gemtuzumab ozogamicin (Mylotarg®), an anti-CD33 (p67 protein) antibody developed by Celltech/Wyeth, alefacept (Amevive®), an anti-LFA-3 Fc fusion developed by Biogen), abciximab (ReoPro®), developed by Centocor/Lilly, basiliximab (Simulect®), developed by Novartis, palivizumab (Synagis®), developed by Medimmune, infliximab (Remicade®), an anti-TNFalpha antibody developed by Centocor, adalimumab (Humira®), an anti-TNFalpha antibody developed by Abbott, Humicade®, an anti-TNFalpha antibody developed by Celltech, golimumab (CNTO-148), a fully human TNF antibody developed by Centocor, etanercept (Enbrel®), an p75 TNF receptor Fc fusion developed by Immunex/Amgen, Ienercept, an p55TNF receptor Fc fusion previously developed by Roche, ABX-CBL, an anti-CD147 antibody being developed by Abgenix, ABX-TL8, an anti-TL8 antibody being developed by Abgenix, ABX-MA1, an anti-MUC18 antibody being developed by Abgenix, Pemtumomab (R1549, 90Y-muHMFG1), an anti-MUC1 in development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegren® (natalizumab), an anti-alpha-4-beta-1 (VLA-4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-βantibody being developed by Cambridge Antibody Technology, ABT 874 (J695), an anti-IL-12 p40 antibody being developed by Abbott, CAT-192, an anti-TGFβ1 antibody being developed by Cambridge Antibody Technology and Genzyme, CAT-213, an anti-Eotaxinl antibody being developed by Cambridge Antibody Technology, LymphoStat-B® an anti-Blys antibody being developed by Cambridge Antibody Technology and Human Genome Sciences Inc., TRAIL-R1 mAb, an anti-TRAIL-R1 antibody being developed by Cambridge Antibody Technology and Human Genome Sciences, Inc., Avastin® bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech, Xolair® (Omalizumab), an anti-IgE antibody being developed by Genentech, Raptiva® (Efalizumab), an anti-CD11a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-IL15, an anti-IL15 antibody being developed by Genmab and Amgen, HuMax-Inflam, being developed by Genmab and Medarex, HuMax-Cancer, an anti-Heparanase I antibody being developed by Genmab and Medarex and Oxford GlycoSciences, HuMax-Lymphoma, being developed by Genmab and Amgen, HuMax-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDEC Pharmaceuticals, IDEC-152, an anti-CD 23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide® (Iabetuzumab), an anti-carcinoembryonic antigen (CEA) antibody being developed by Immunomedics, LymphoCide® (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, Osidem® (IDM-1), and anti-Her2 antibody being developed by Medarex and Immuno-Designed Molecules, HuMax®-CD4, an anti-CD4 antibody being developed by Medarex and Genmab, HuMax-IL15, an anti-IL15 antibody being developed by Medarex and Genmab, CNTO 148, an anti-TNFα antibody being developed by Medarex and Centocor/J&J, CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumab), an anti-CD3 antibody being developed by Protein Design Labs, HuZAF®, an anti-gamma interferon antibody being developed by Protein Design Labs, Anti-α5β1 Integrin, being developed by Protein Design Labs, anti-IL-12, being developed by Protein Design Labs, ING-1, an anti-Ep-CAM antibody being developed by Xoma, Xolair® (Omalizumab) a humanized anti-IgE antibody developed by Genentech and Novartis, and MLNO1, an anti-Beta2 integrin antibody being developed by Xoma.

In another embodiment, the therapeutics include KRN330 (Kirin); huA33 antibody (A33, Ludwig Institute for Cancer Research); CNTO 95 (alpha V integrins, Centocor); MEDI-522 (alpha Vβ3integrin, Medimmune); volociximab (alpha Vβ1 integrin, Biogen/PDL); Human mAb 216 (B cell glycosolated epitope, NCl); BiTE MT103 (bispecific CD19×CD3, Medimmune); 4G7×H22 (Bispecific BcellxFcgammaRl, Medarex/Merck Kga); rM28 (Bispecific CD28×MAPG, EP Patent No. EP1444268); MDX447 (EMD 82633) (Bispecific CD64×EGFR, Medarex); Catumaxomab (removab) (Bispecific EpCAMx anti-CD3, Trion/Fres); Ertumaxomab (bispecific HER2/CD3, Fresenius Biotech); oregovomab (OvaRex) (CA-125, ViRexx); Rencarex® (WX G250) (carbonic anhydrase IX, Wilex); CNTO 888 (CCL2, Centocor); TRC105 (CD105 (endoglin), Tracon); BMS-663513 (CD137 agonist, Bristol Myers Squibb); MDX-1342 (CD19, Medarex); Siplizumab (MEDI-507) (CD2, Medimmune); Ofatumumab (Humax-CD20) (CD20, Genmab); Rituximab (Rituxan) (CD20, Genentech); veltuzumab (hA20) (CD20, Immunomedics); Epratuzumab (CD22, Amgen); lumiliximab (IDEC 152) (CD23, Biogen); muromonab-CD3 (CD3, Ortho); HuM291 (CD3 fc receptor, PDL Biopharma); HeFi-1, CD30, NCl); MDX-060 (CD30, Medarex); MDX-1401 (CD30, Medarex); SGN-30 (CD30, Seattle Genentics); SGN-33 (Lintuzumab) (CD33, Seattle Genentics); Zanolimumab (HuMax-CD4) (CD4, Genmab); HCD122 (CD40, Novartis); SGN-40 (CD40, Seattle Genentics); MabCampath (Alemtuzumab) (CD52, Genzyme); MDX-1411 (CD70, Medarex); hLL1 (EPB-1) (CD74.38, Immunomedics); Galiximab (IDEC-144) (CD80, Biogen); MT293 (TRC093/D93) (cleaved collagen, Tracon); HuLuc63 (CS1, PDL Pharma); ipilimumab (MDX-010) (CTLA4, Bristol Myers Squibb); Tremelimumab (Ticilimumab, CP-675,2) (CTLA4, Pfizer); HGS-ETR1 (Mapatumumab) (DR4TRAIL-R1 agonist, Human Genome Science/Glaxo Smith Kline); AMG-655 (DR5, Amgen); Apomab (DR5, Genentech); CS-1008 (DR5, Daiichi Sankyo); HGS-ETR2 (lexatumumab) (DR5TRAIL-R2 agonist, HGS); Cetuximab (Erbitux) (EGFR, Imclone); IMC-11F8, (EGFR, Imclone); Nimotuzumab (EGFR, YM Bio); Panitumumab (Vectabix) (EGFR, Amgen); Zalutumumab (HuMaxEGFr) (EGFR, Genmab); CDX-110 (EGFRvIII, AVANT Immunotherapeutics); adecatumumab (MT201) (Epcam, Merck); edrecolomab (Panorex, 17-1A) (Epcam, Glaxo/Centocor); MORAb-003 (folate receptor a, Morphotech); KW-2871 (ganglioside GD3, Kyowa); MORAb-009 (GP-9, Morphotech); CDX-1307 (MDX-1307) (hCGb, Celldex); Trastuzumab (Herceptin) (HER2, Celldex); Pertuzumab (rhuMAb 2C4) (HER2 (DI), Genentech); apolizumab (HLA-DR beta chain, PDL Pharma); AMG-479 (IGF-1R, Amgen); anti-IGF-1RR1507 (IGF1-R, Roche); CP 751871 (IGF1-R, Pfizer); IMC-A12 (IGF1-R, Imclone); BIIB022 (IGF-1R, Biogen); Mik-beta-1 (IL-2Rb (CD122), Hoffman-La Roche); CNTO 328 (IL6, Centocor); Anti-KIR (1-7F9) (Killer cell Ig-like Receptor (KIR), Novo); Hu3S193 (Lewis (y), Wyeth, Ludwig Institute of Cancer Research); hCBE-11 (LTOR, Biogen); HuHMFGI (MUC1, Antisoma/NCl); RAV12 (N-linked carbohydrate epitope, Raven); CAL (parathyroid hormone-related protein (PTH-rP), University of California); CT-011 (PD1, CureTech); MDX-1106 (ono-4538) (PD1, Medarex/Ono); Mab CT-011 (PD1, Curetech); IMC-3G3 (PDGFRa, Imclone); bavituximab (phosphatidylserine, Peregrine); huJ591 (PSMA, Cornell Research Foundation); muJ591 (PSMA, Cornell Research Foundation); GC1008 (TGFb (pan) inhibitor (IgG4), Genzyme); Infliximab (Remicade) (TNFα, Centocor); A27.15 (transferrin receptor, Salk Institute, INSERN WO 2005/111082); E2.3 (transferrin receptor, Salk Institute); Bevacizumab (Avastin) (VEGF, Genentech); HuMV833 (VEGF, Tsukuba Research Lab, PCT Publication No. WO/2000/034337, University of Texas); IMC-18F1 (VEGFR1, Imclone); IMC-1121 (VEGFR2, Imclone).

Examples of useful bispecific antibodies include, but are not limited to, those with one antibody directed against a tumor cell antigen and the other antibody directed against a cytotoxic trigger molecule such as anti-FcγRI/anti-CD 15, anti-p185^{HER2}/FcγRIII (CD16), anti-CD3/anti-malignant B-cell (1D10), anti-CD3/anti-p185^{HER2}, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell adhesion molecule (NCAM)/anti-CD3, anti-folate binding protein (FBP)/anti-CD3, anti-pan carcinoma associated antigen (AMOC-31)/anti-CD3; bispecific antibodies with one antibody which binds specifically to a tumor antigen and another antibody which binds to a toxin such as anti-saporin/anti-Id-1, anti-CD22/anti-saporin, anti-CD7/anti-saporin, anti-CD38/anti-saporin, anti-CEA/anti-ricin A chain, anti-interferon-α (IFN-α)/anti-hybridoma idiotype, anti-CEA/anti-vinca alkaloid; bispecific antibodies for converting enzyme activated prodrugs such as anti-CD30/anti-alkaline phosphatase (which catalyzes conversion of mitomycin phosphate prodrug to mitomycin alcohol); bispecific antibodies which can be used as fibrinolytic agents such as anti-fibrin/anti-tissue plasminogen activator (tPA), anti-fibrin/anti-urokinase-type plasminogen activator (uPA); bispecific antibodies for targeting immune complexes to cell surface receptors such as anti-low density lipoprotein (LDL)/anti-Fc receptor (e.g., FcγRI, FcγRII or FcγRIII); bispecific antibodies for use in therapy of infectious diseases such as anti-CD3/anti-herpes simplex virus (HSV), anti-T-cell receptor:CD3 complex/anti-influenza, anti-FcγR/anti-HIV; bispecific antibodies for tumor detection in vitro or in vivo such as anti-CEA/anti-EOTUBE, anti-CEA/anti-DPTA, anti-anti-p185^{HER2}/anti-hapten; bispecific antibodies as vaccine adjuvants (see Fanger, M W et al., Crit Rev Immunol. 1992; 12(34): 101-24, which is incorporated by reference herein); and bispecific antibodies as diagnostic tools such as anti-rabbit IgG/anti-ferritin, anti-horse radish peroxidase (HIRP)/anti-hormone, anti-somatostatin/anti -substance P, anti-TIRP/anti-FITC, anti -CEA/anti-β-galactosidase (see Nolan, O, and O'Kennedy, R., Biochim Biophys Acta. 1990 Aug. 1; 1040(1): 1-11, which is incorporated by reference herein). Examples oftispecific antibodies include anti-CD3/anti-CD4/anti-CD37, anti-CD3/anti-CD 5/anti-CD37 and anti-CD3/anti-CD8/anti-CD37.

In one embodiment, the conjugate of Formula (CONJ-I)—(CONJ-VA) is a conjugate of Table C or Table C-1 or a pharmaceutically acceptable salt or tautomer thereof:

TABLE C:

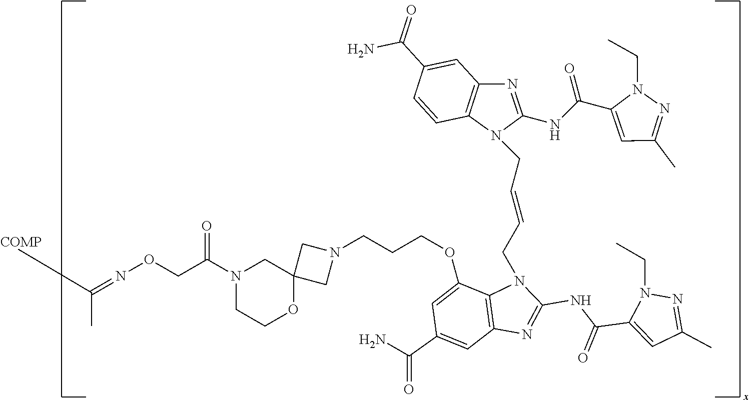

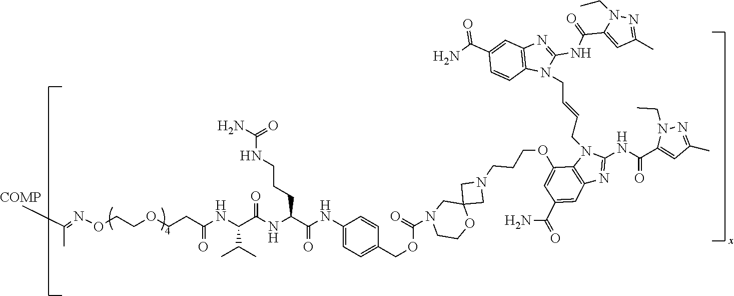

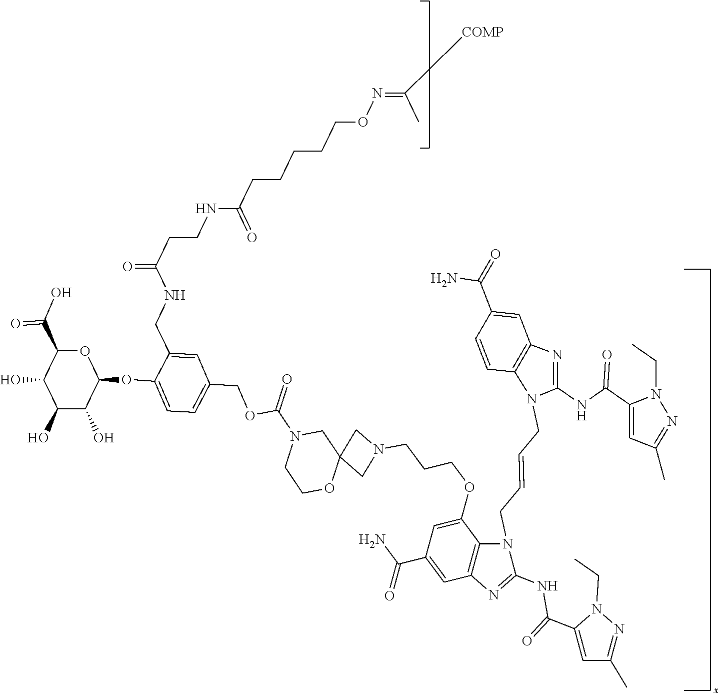

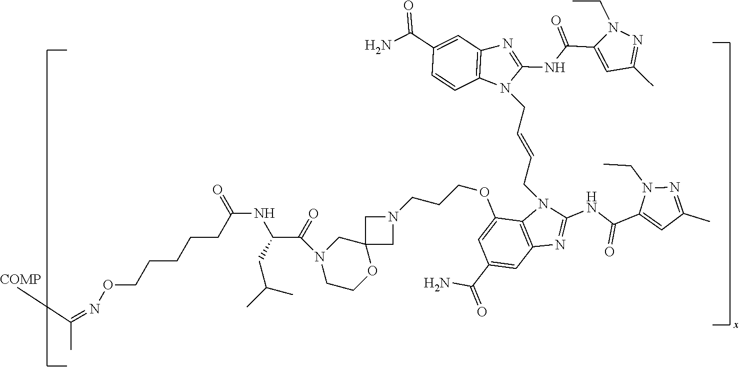

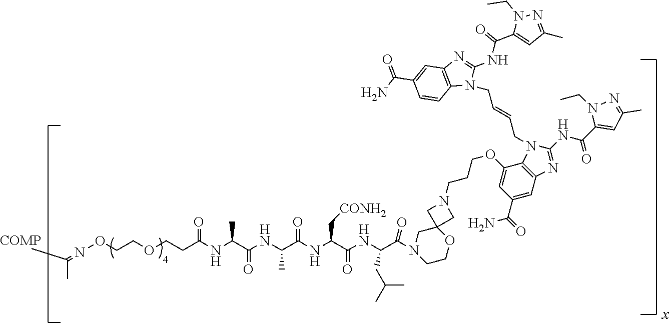

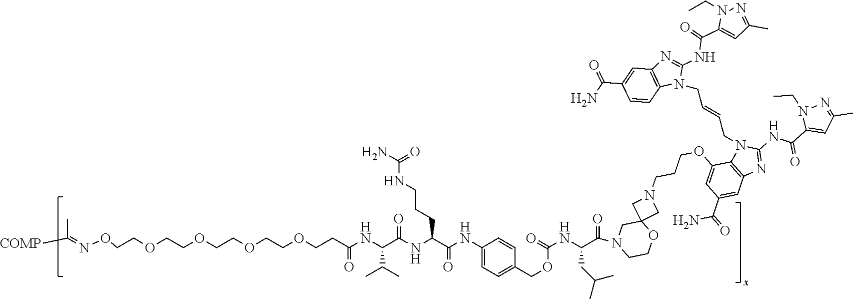

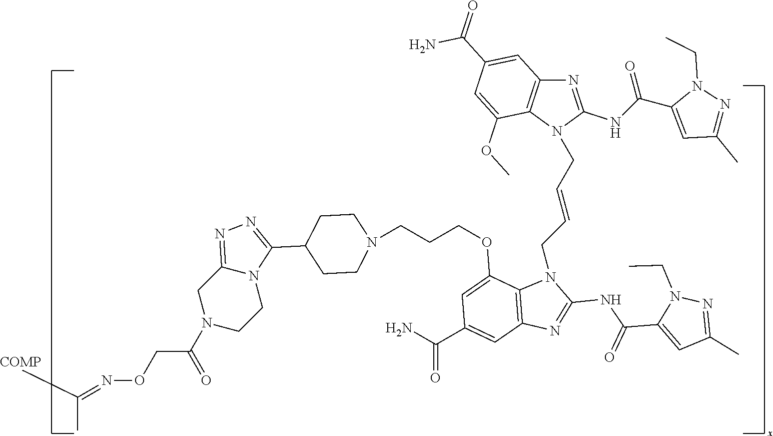

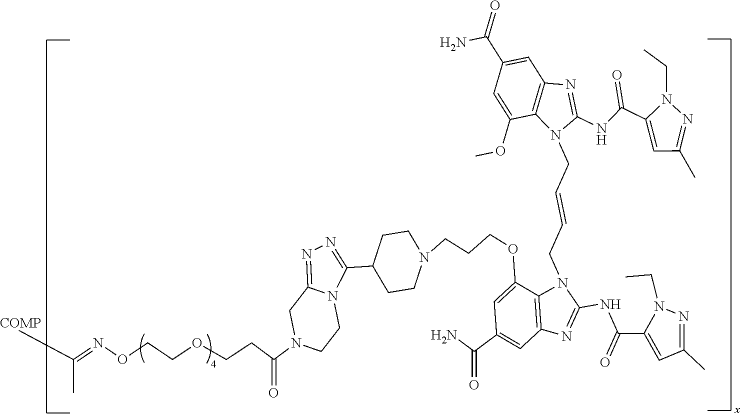

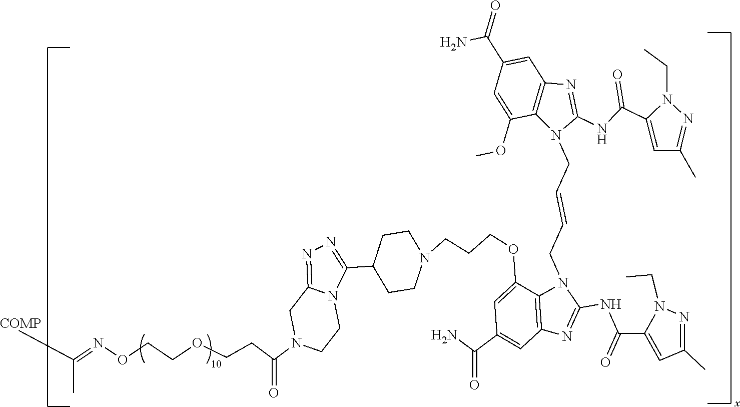

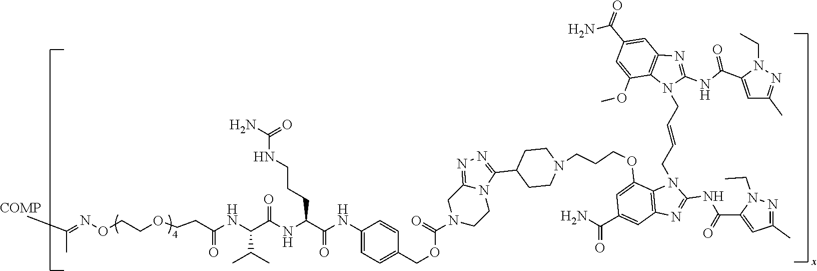

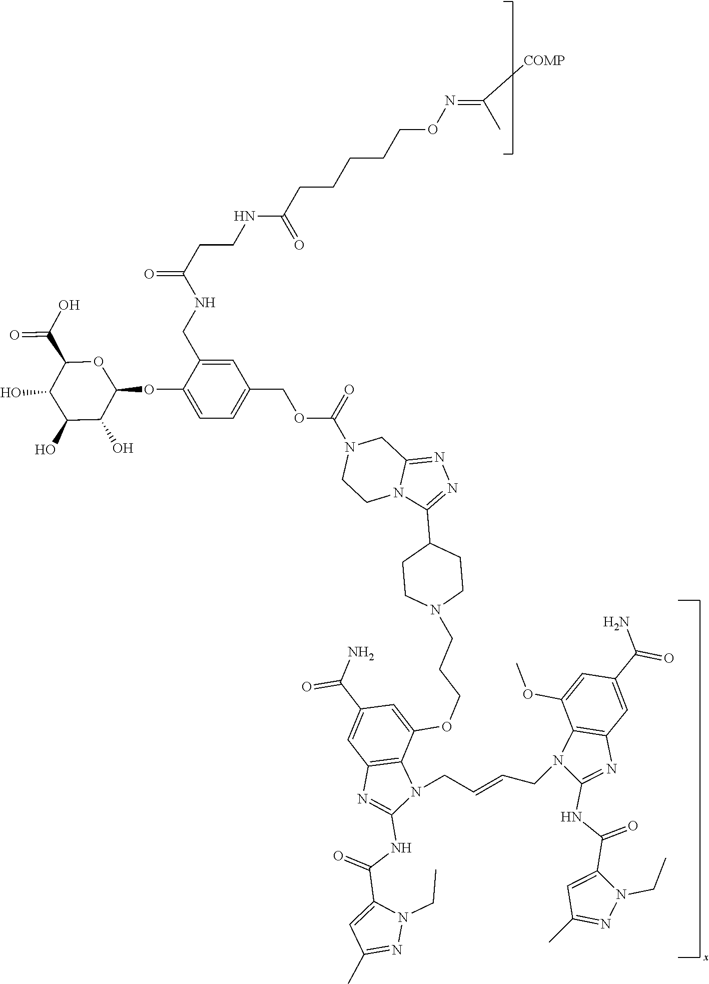

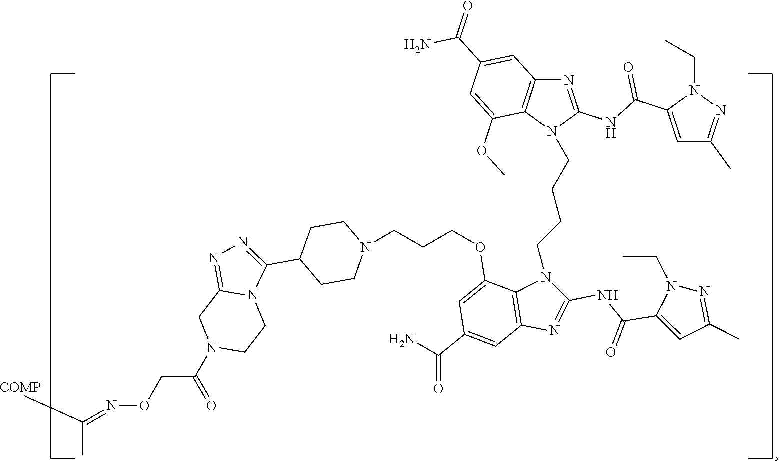

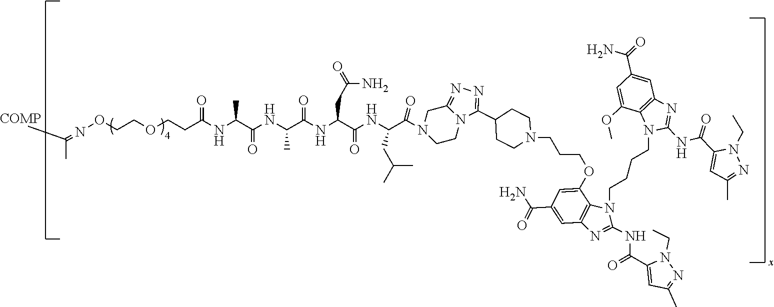

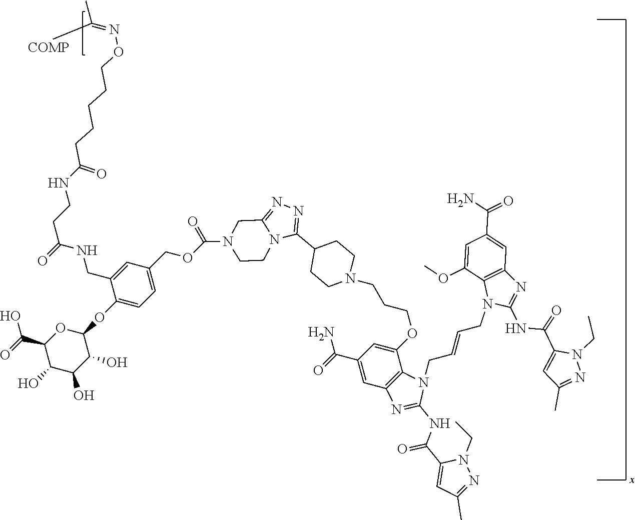

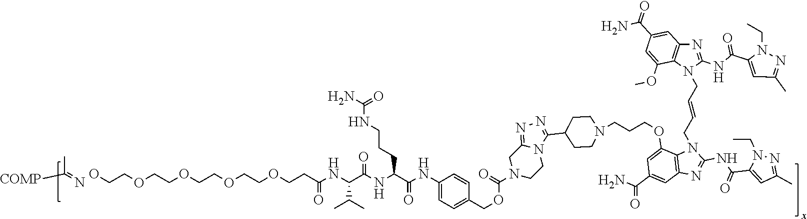

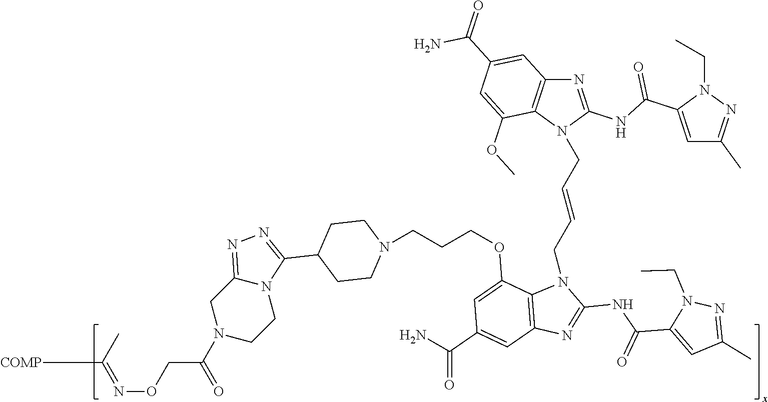

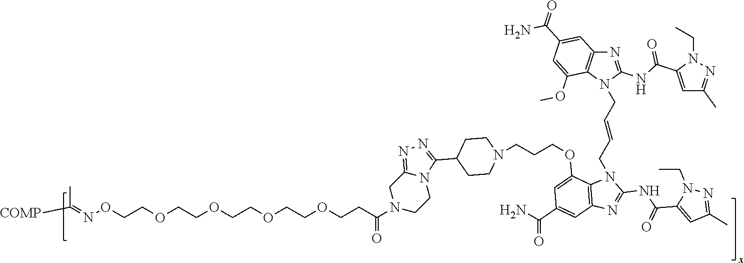

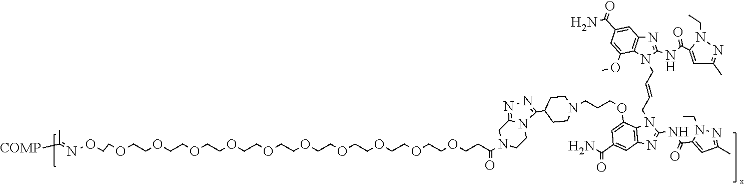

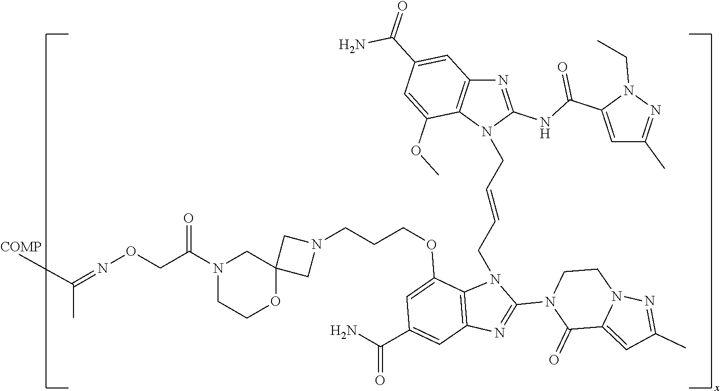

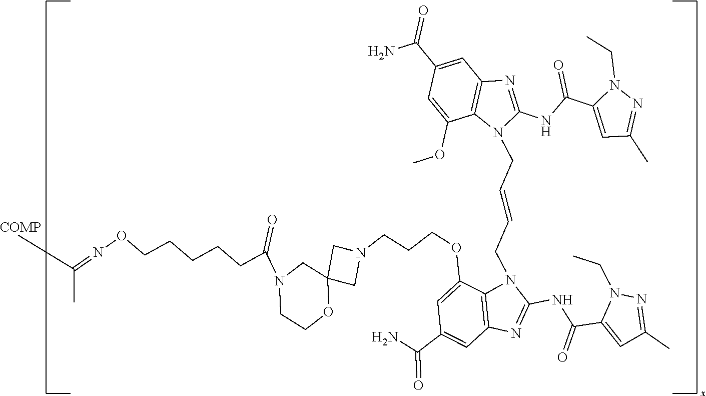

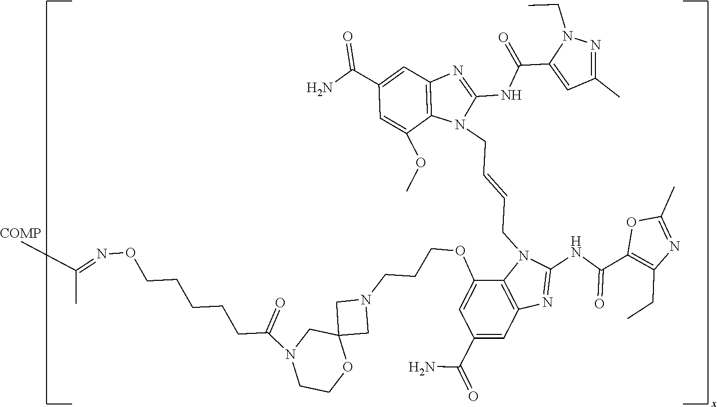

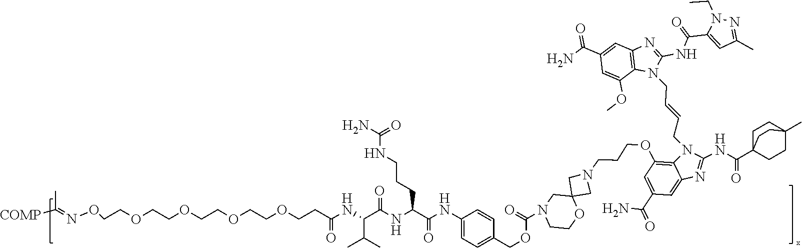

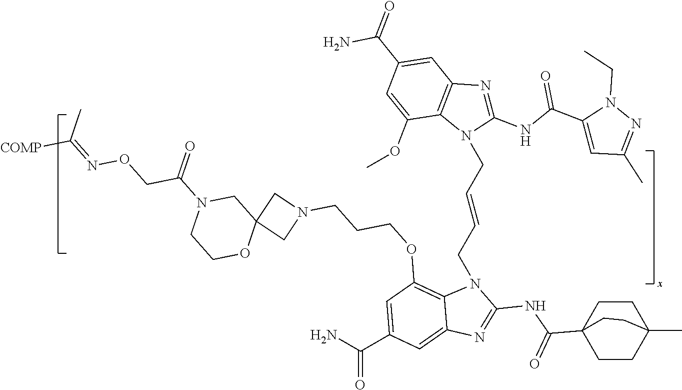

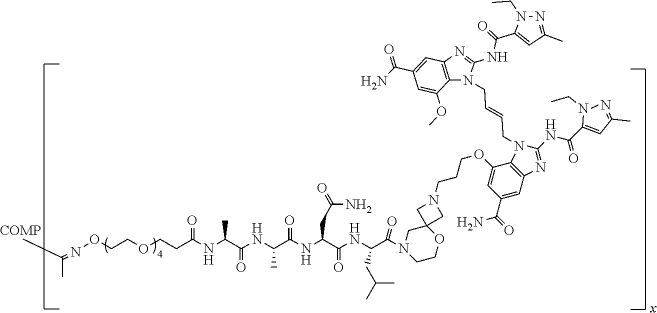

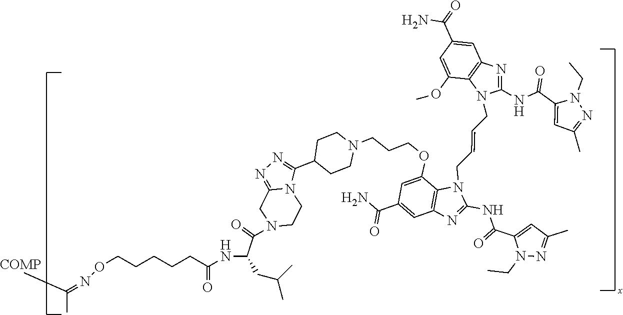

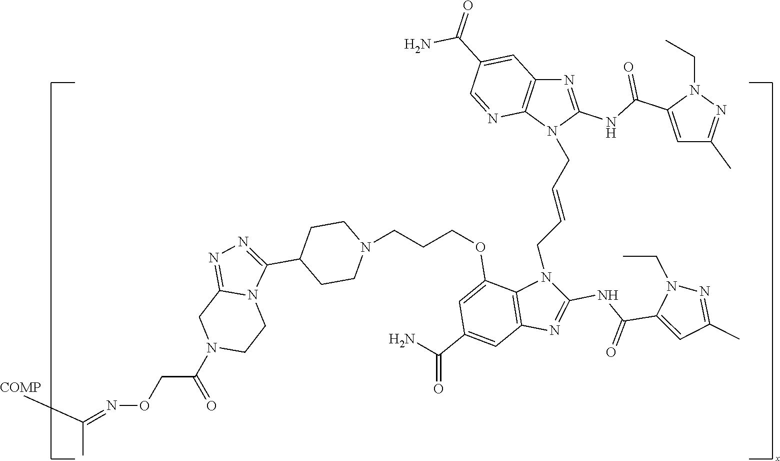

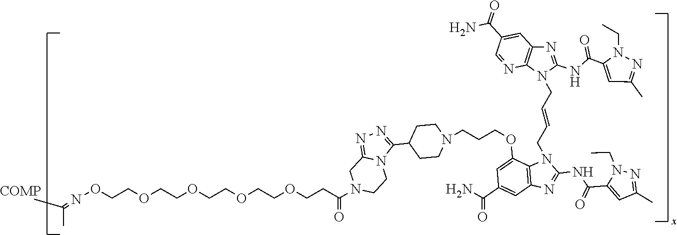

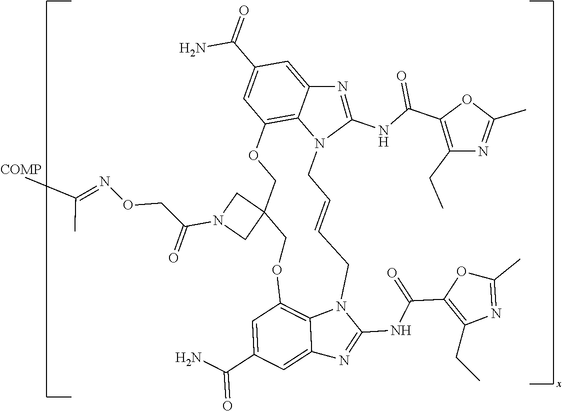

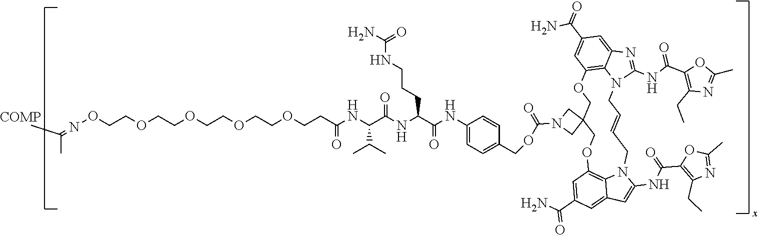

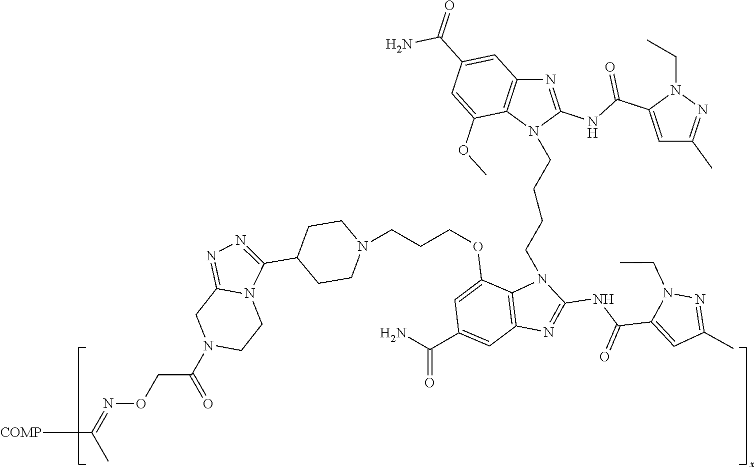

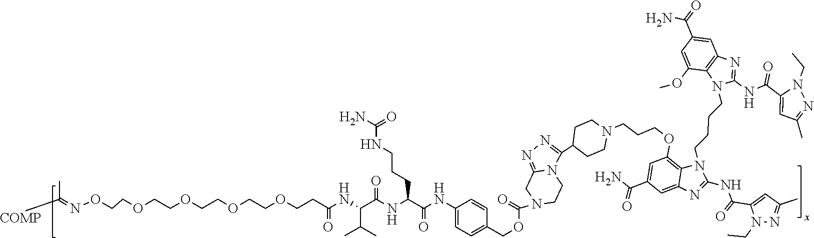

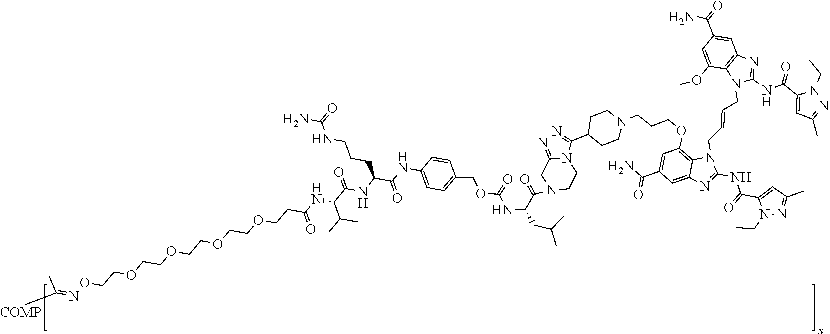

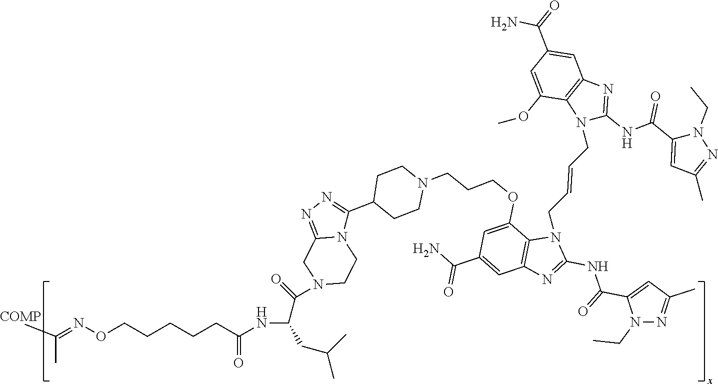

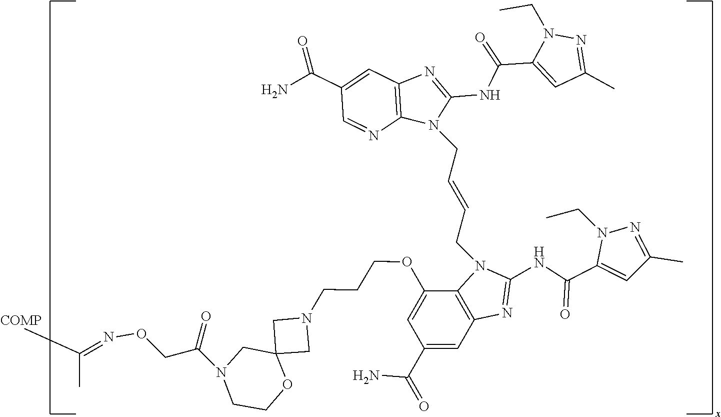

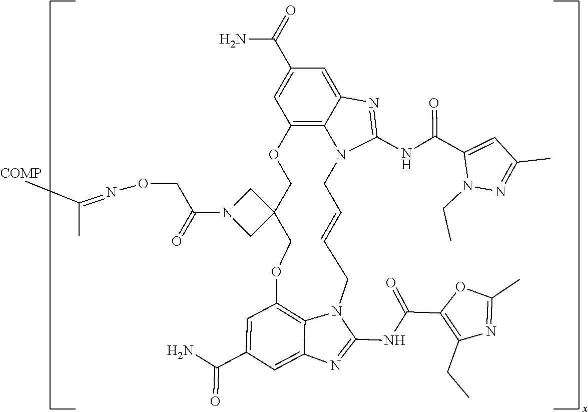

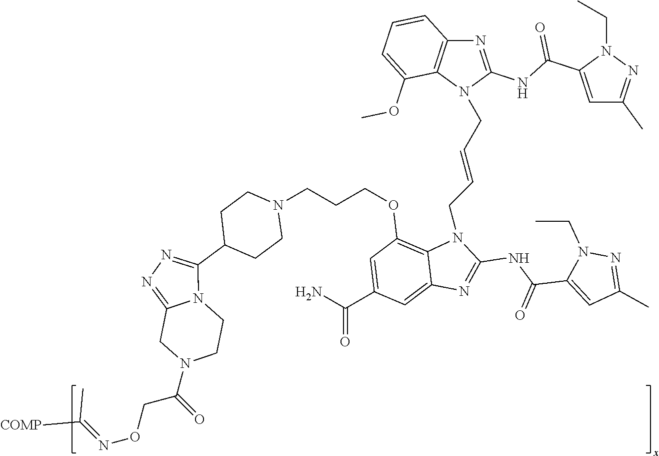

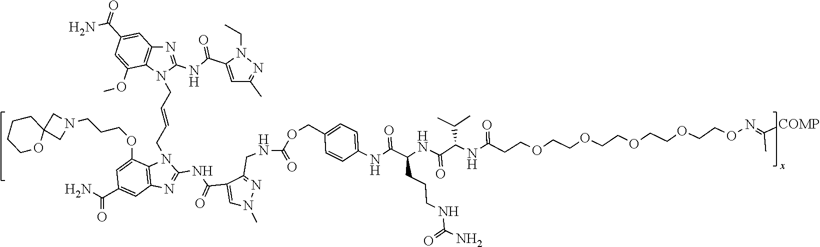

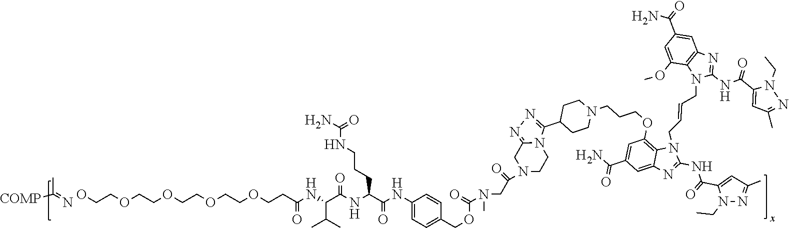

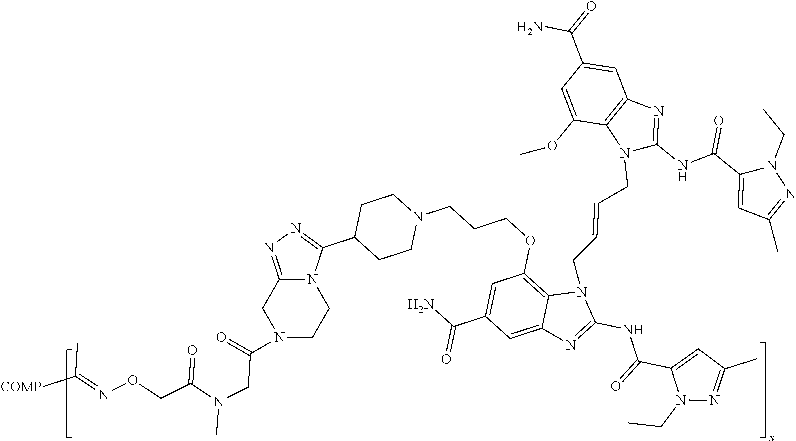

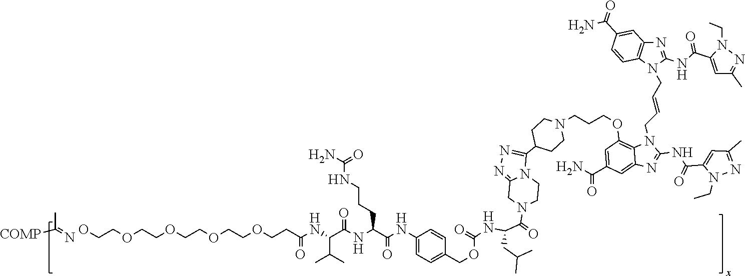

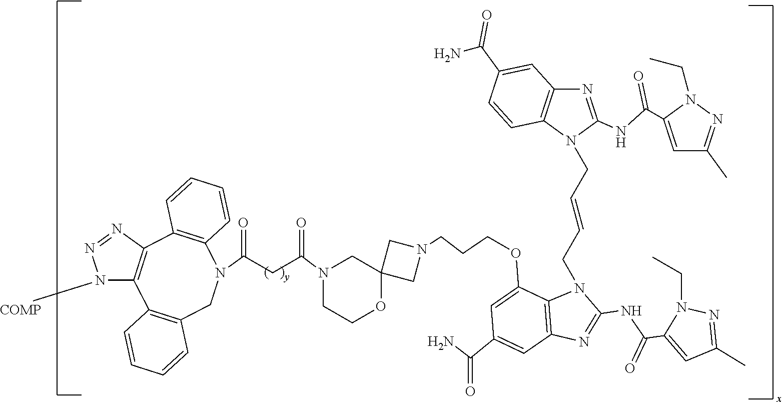

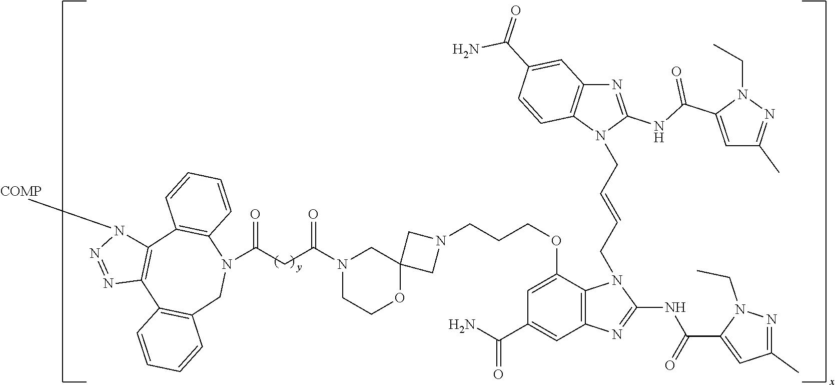

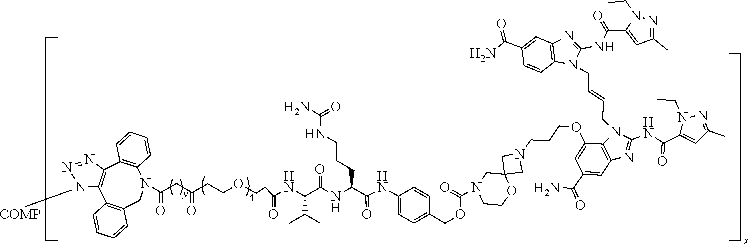

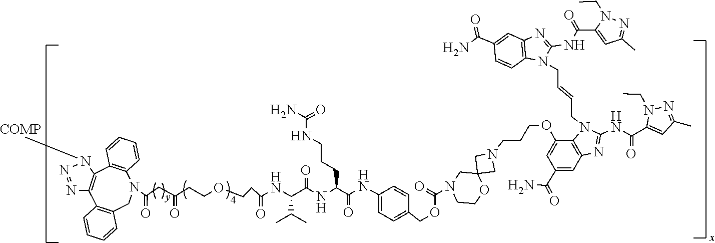

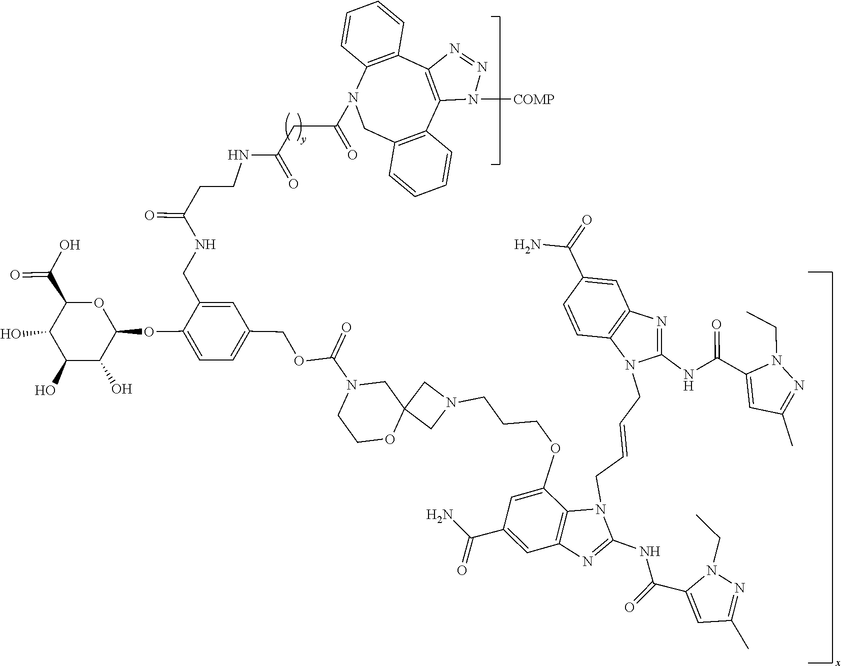

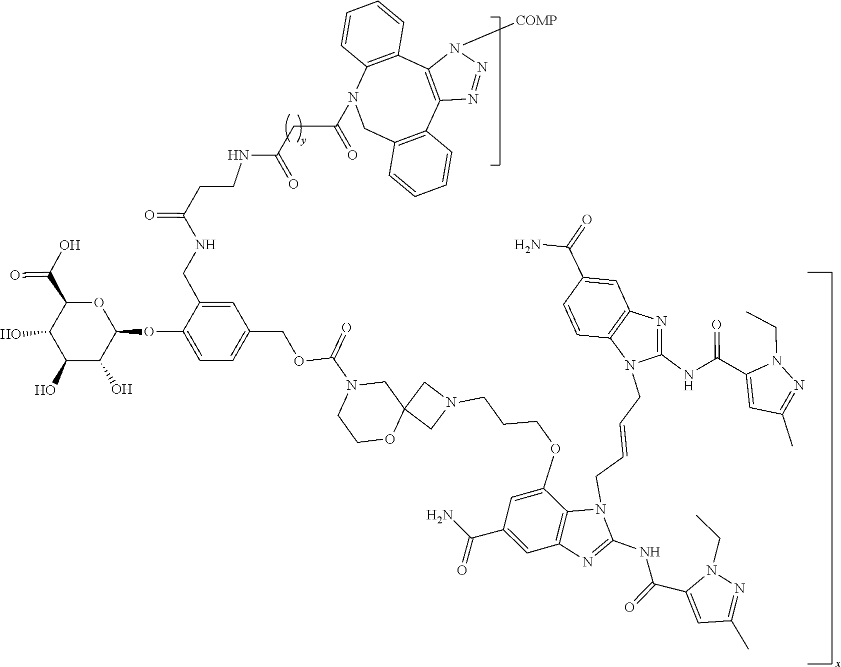

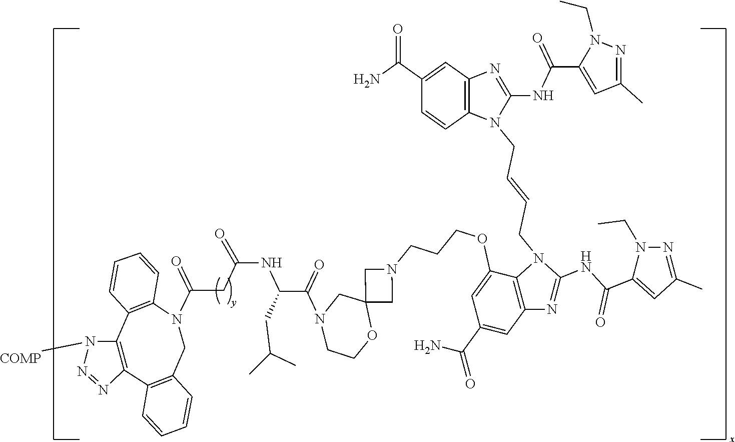

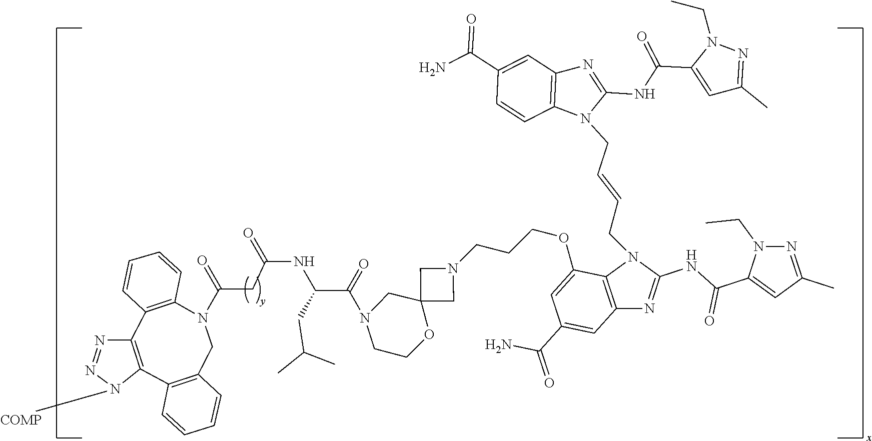

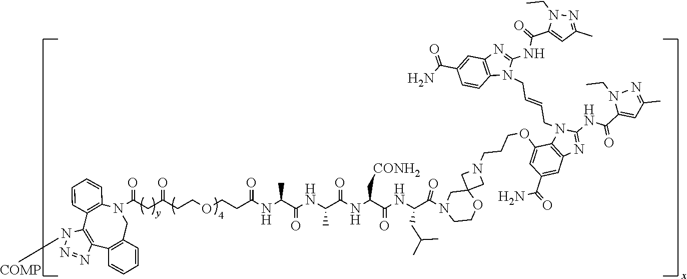

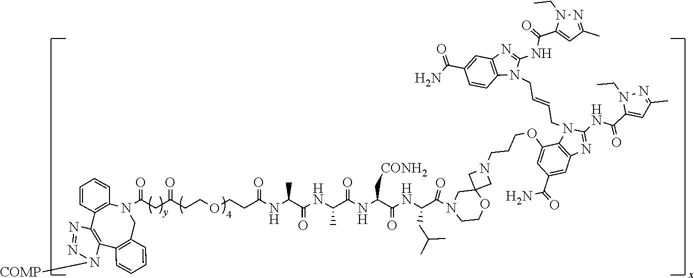

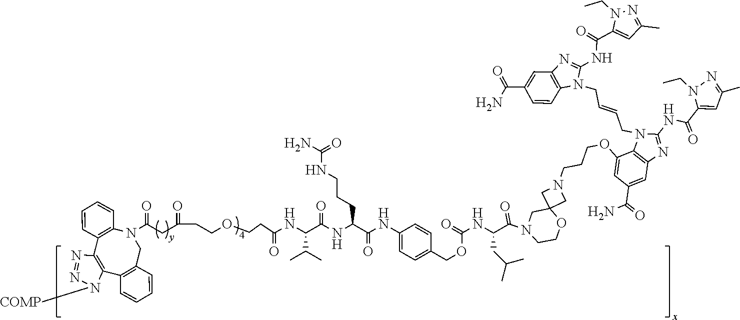

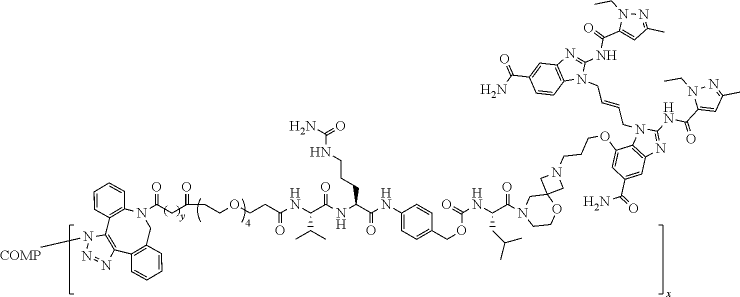

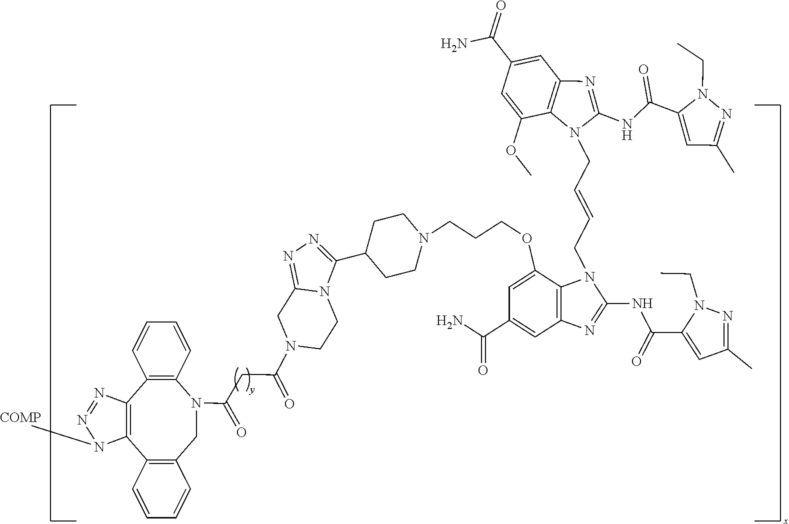

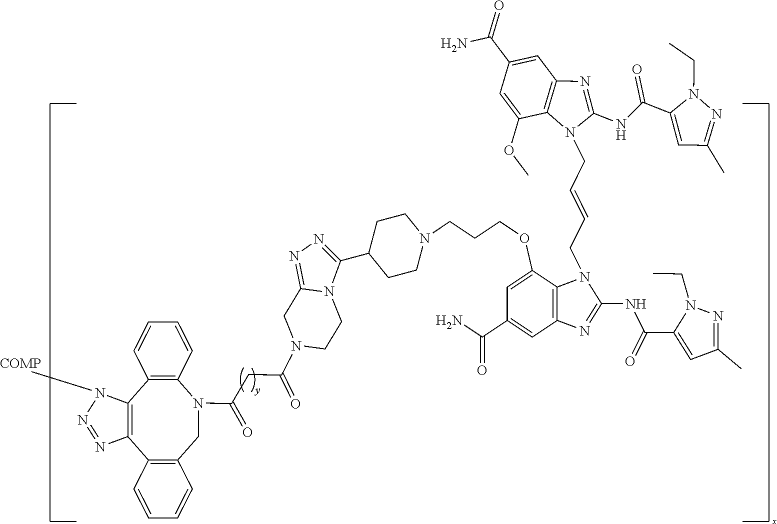

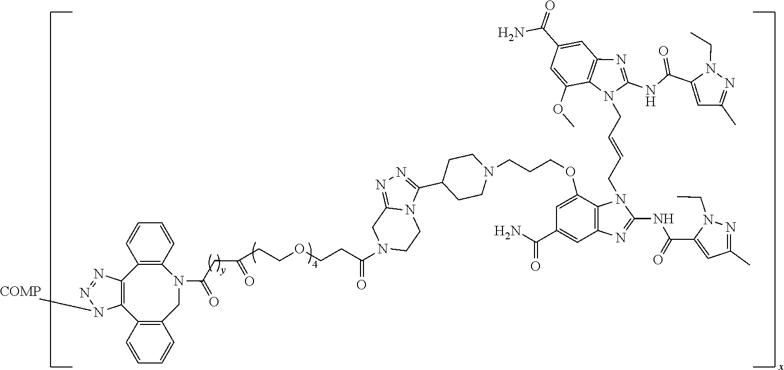

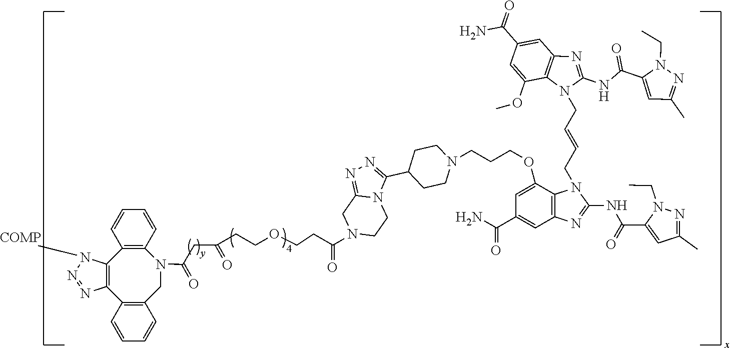

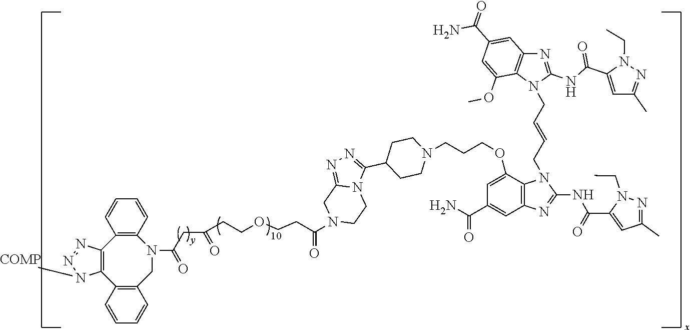

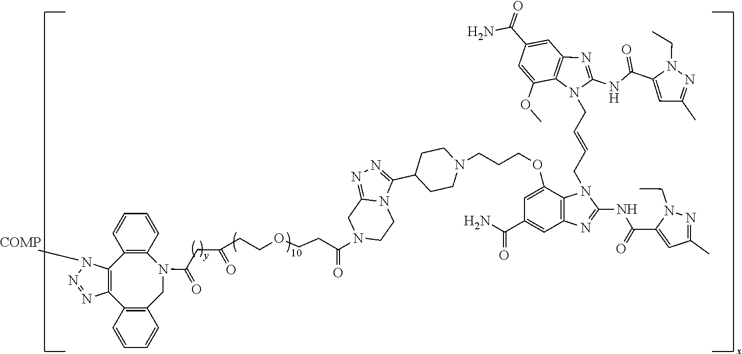

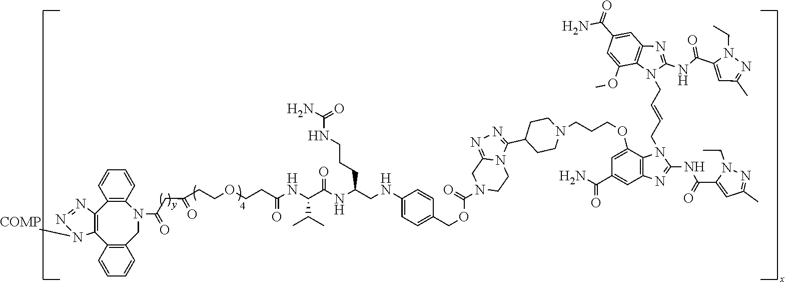

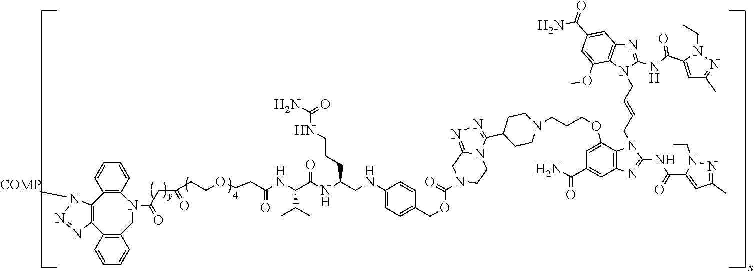

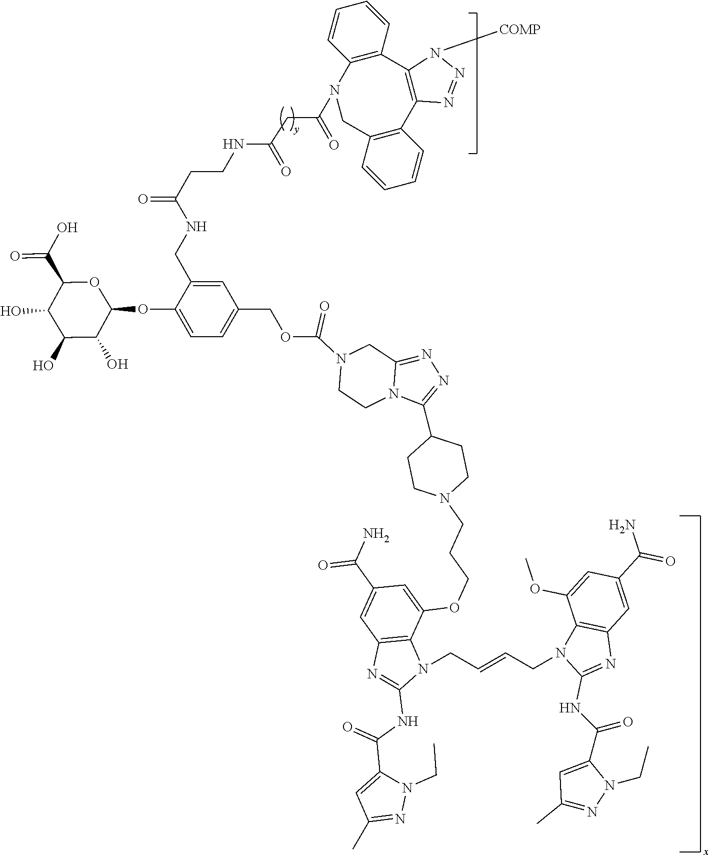

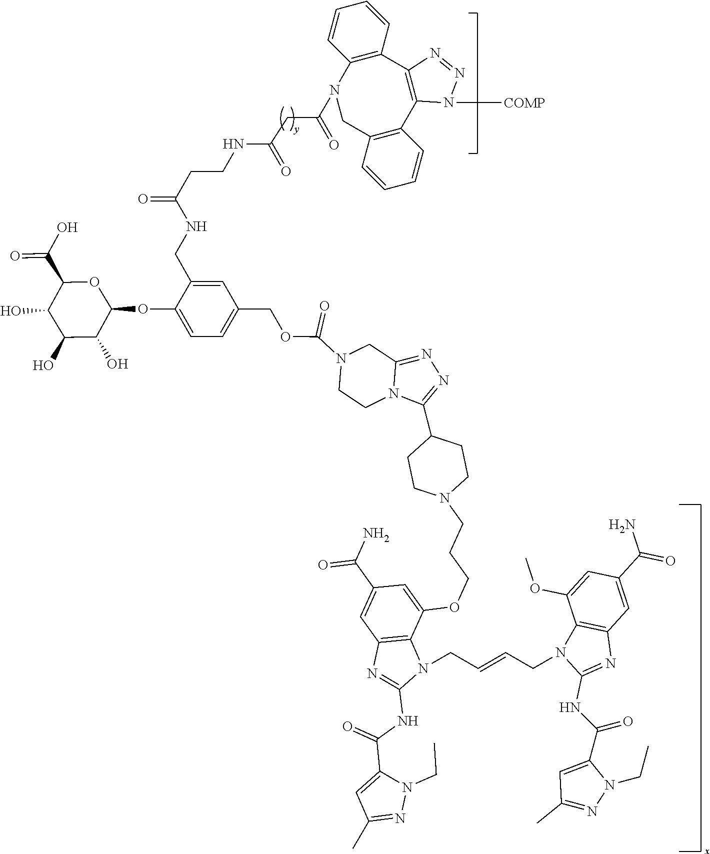

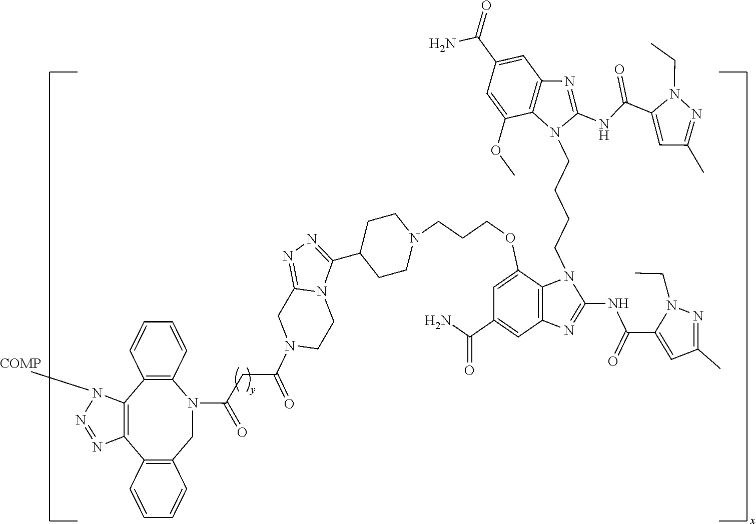

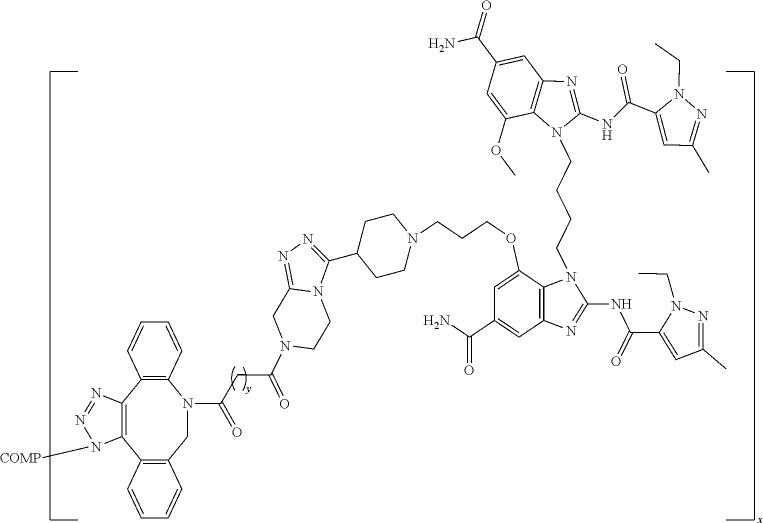

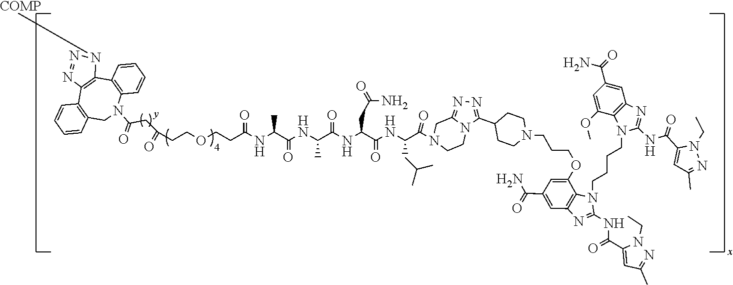

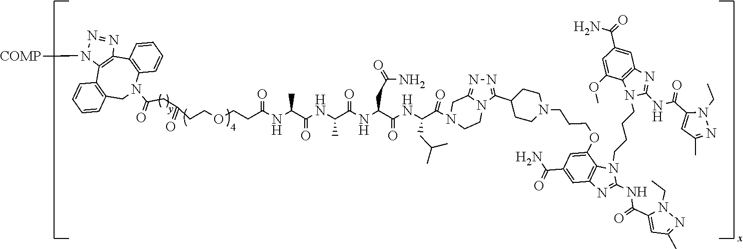

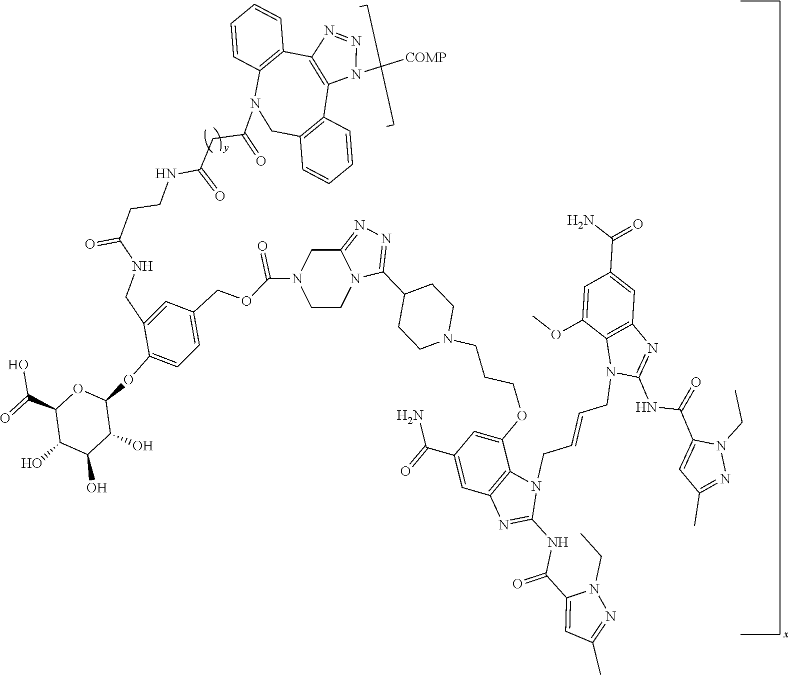

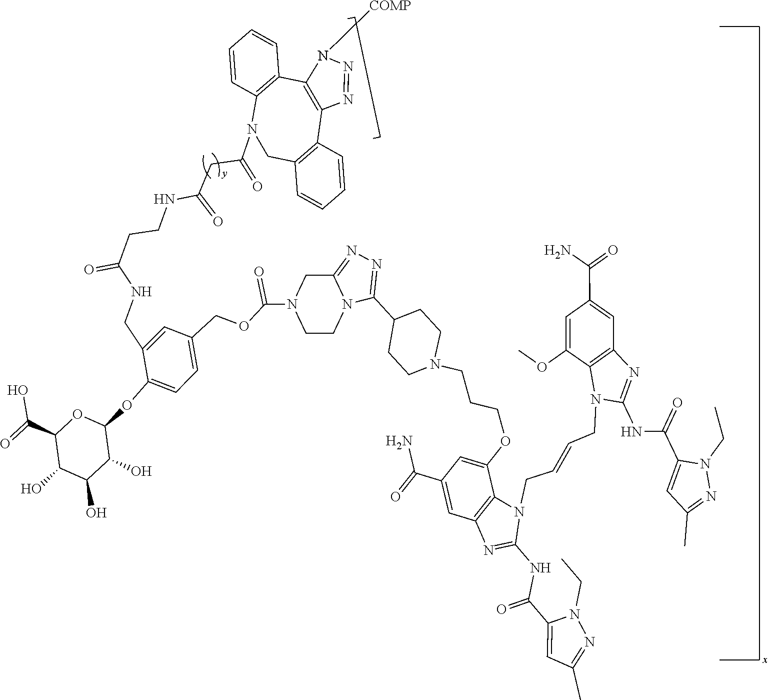

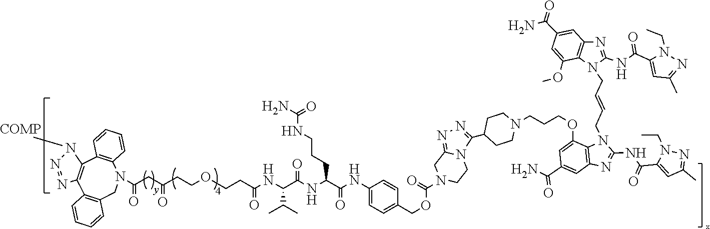

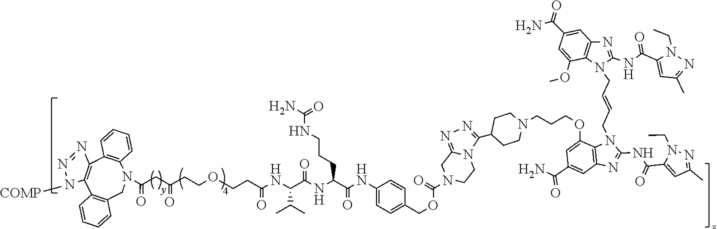

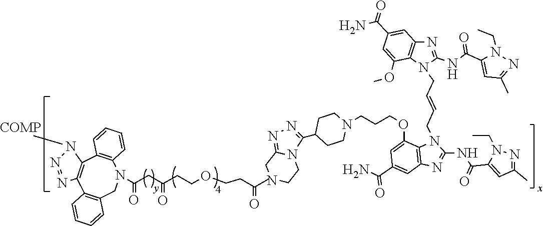

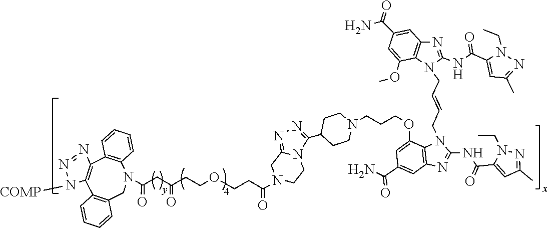

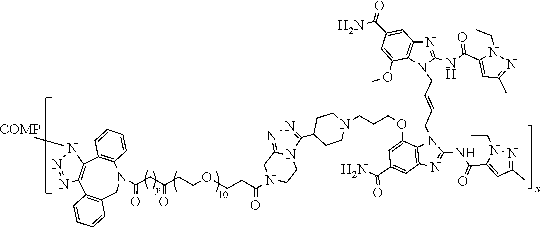

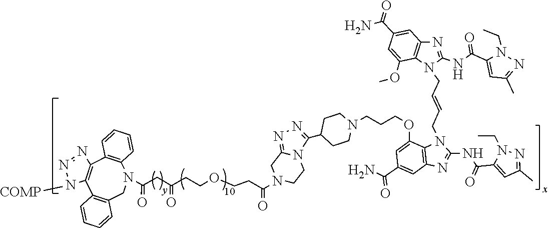

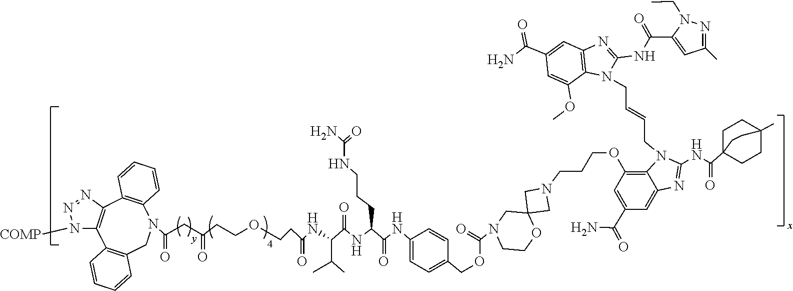

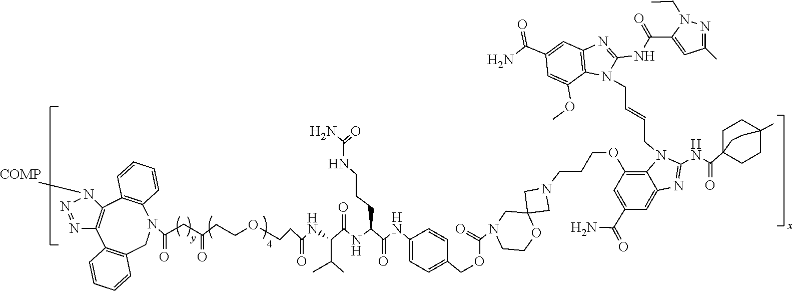

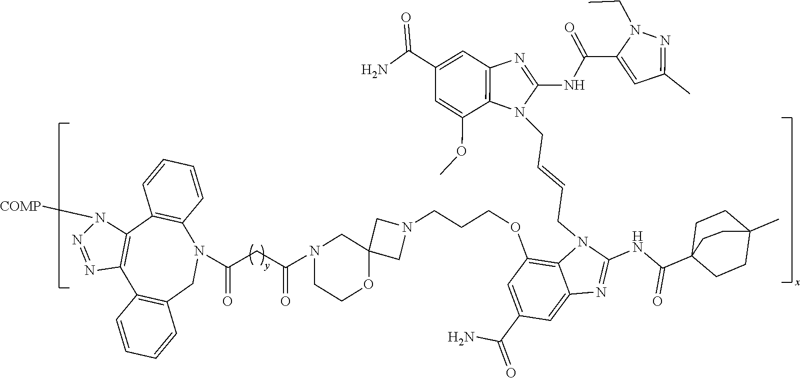

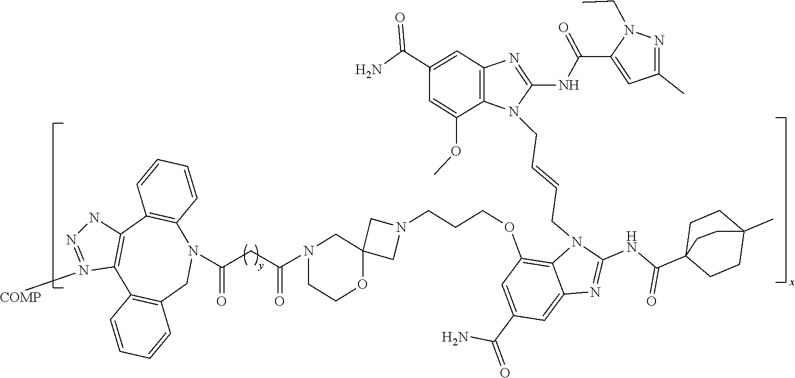

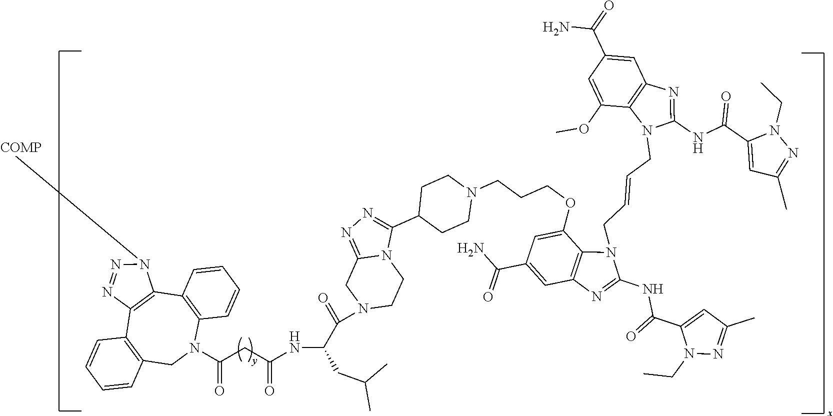

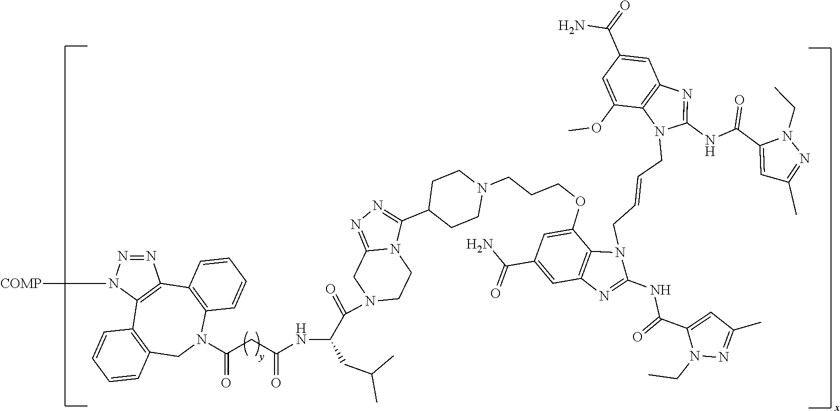

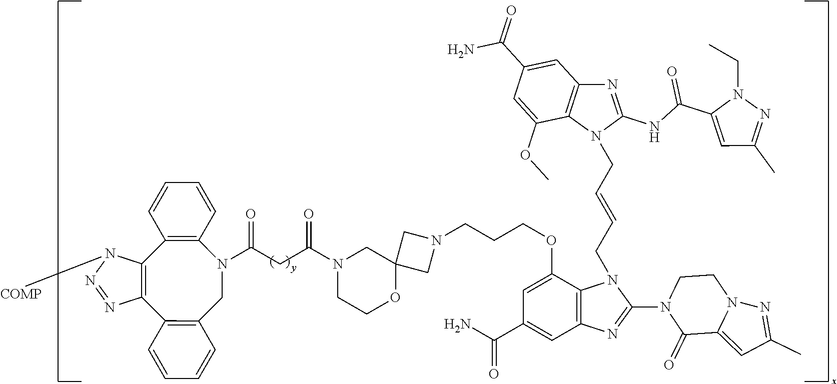

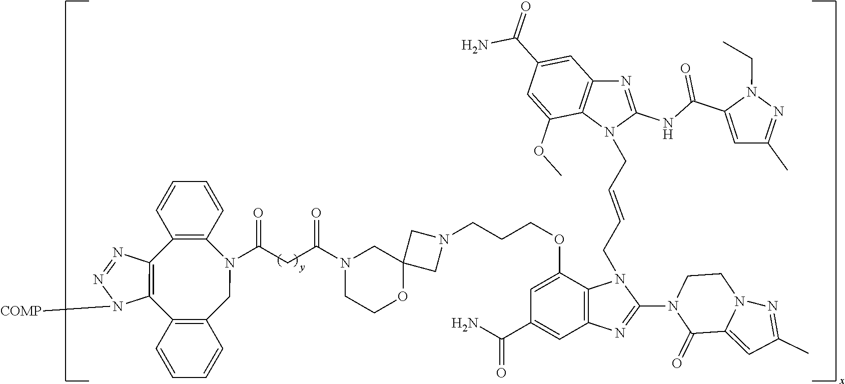

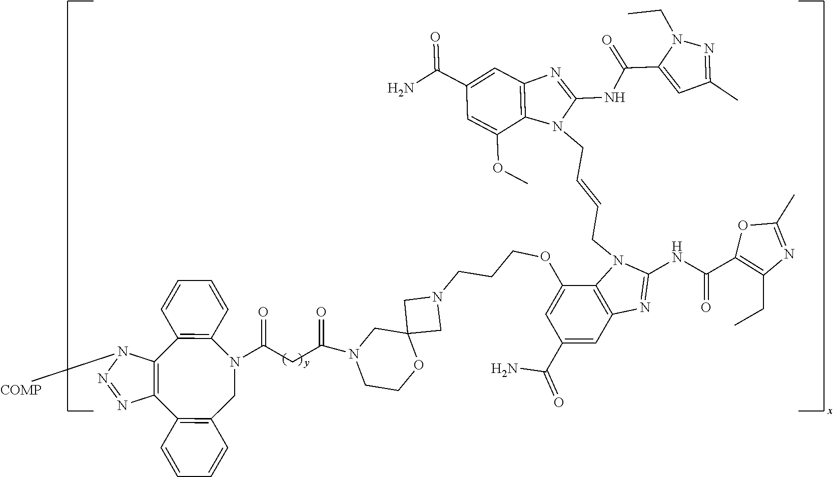

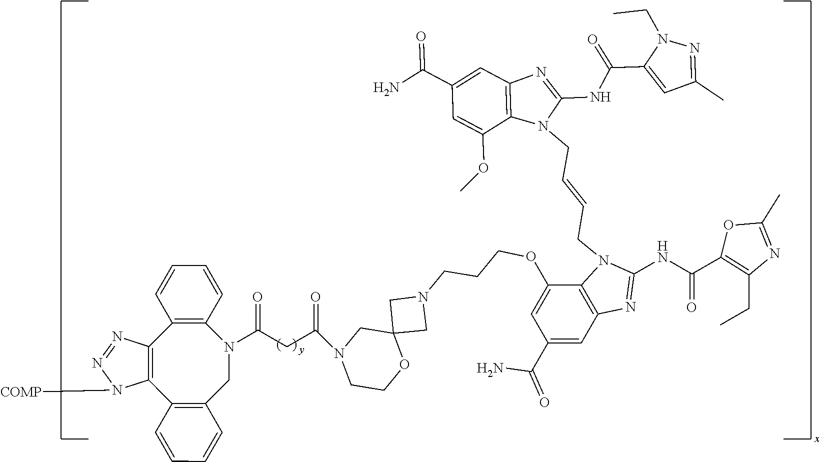

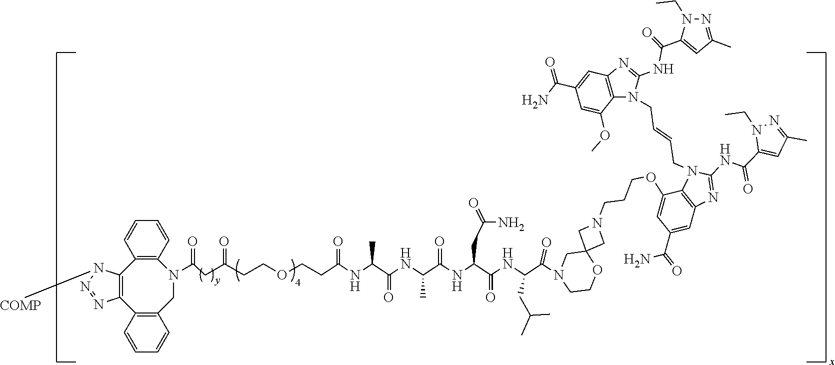

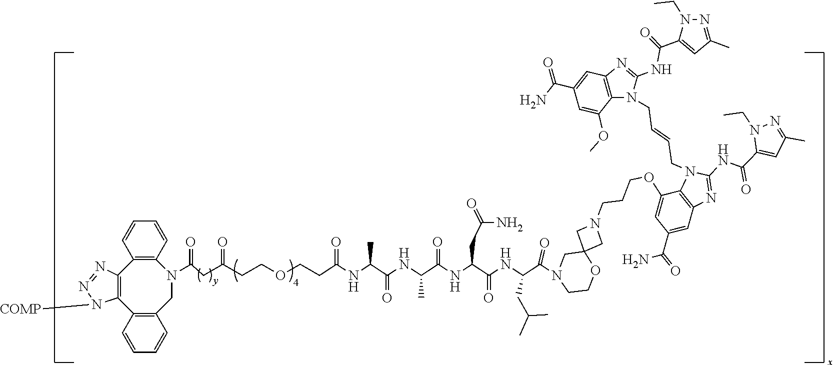

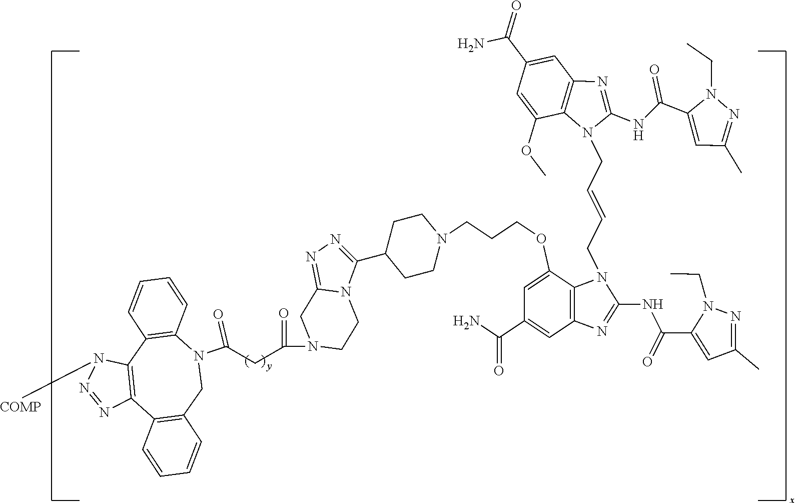

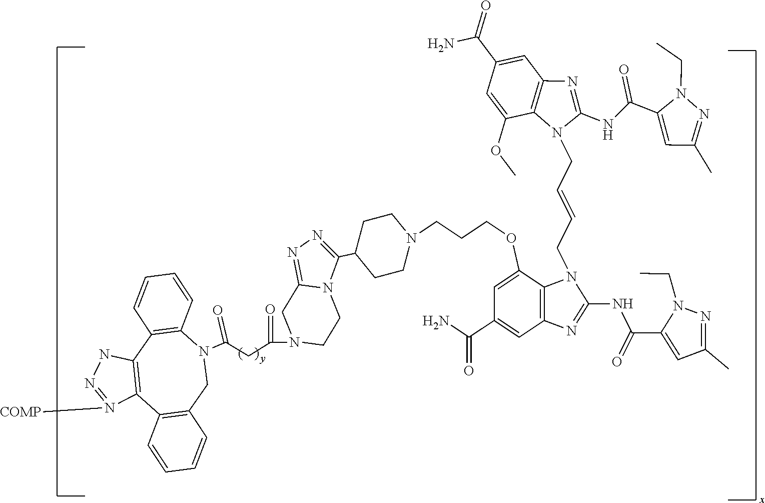

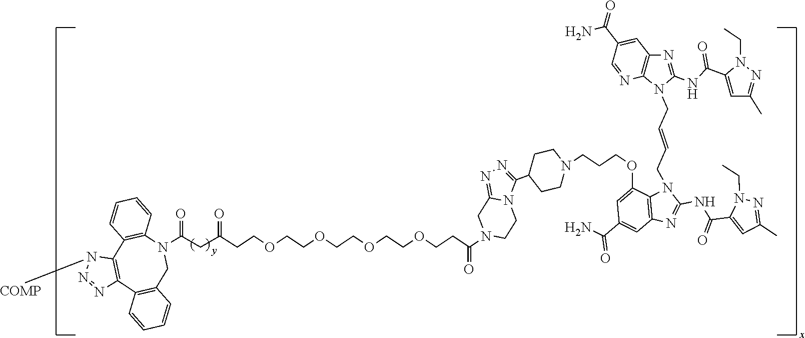

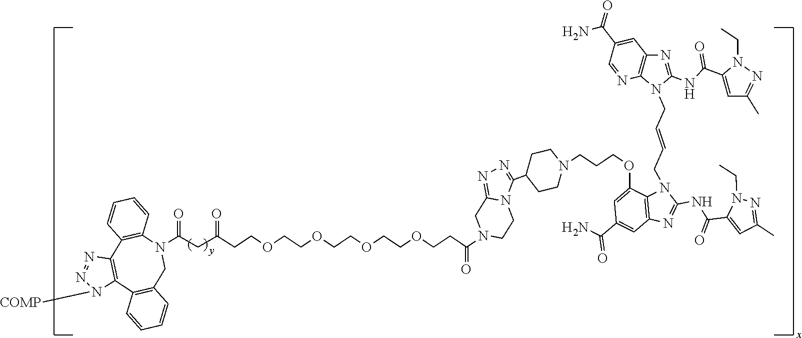

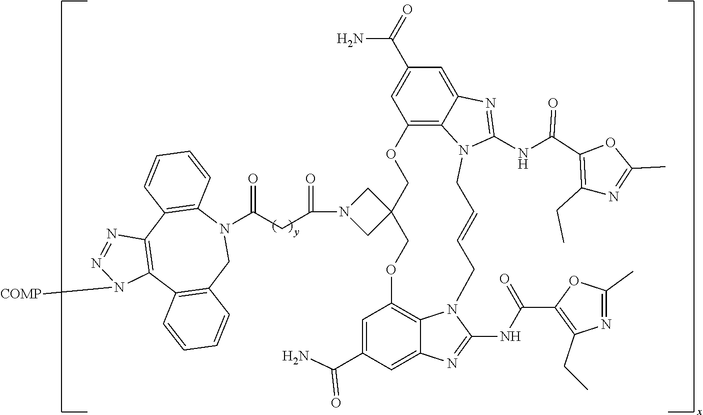

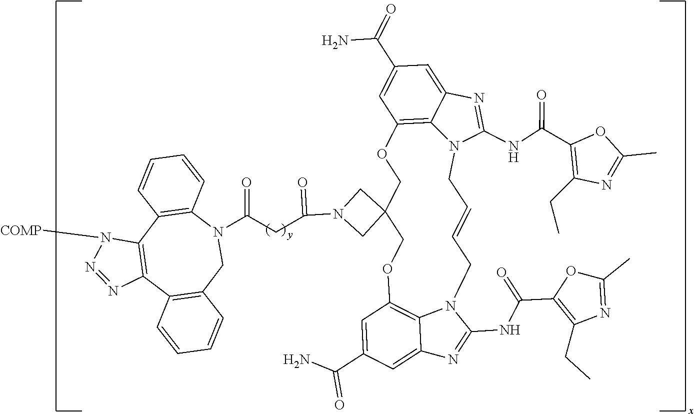

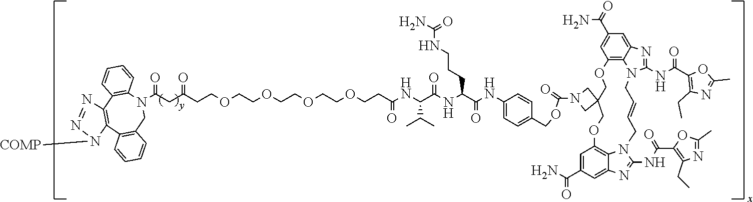

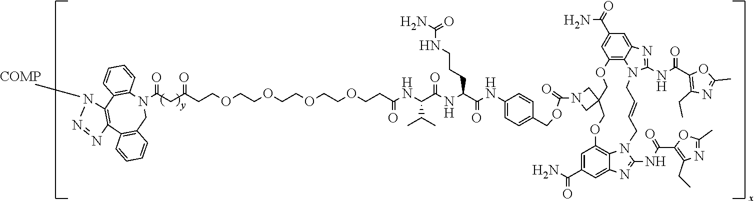

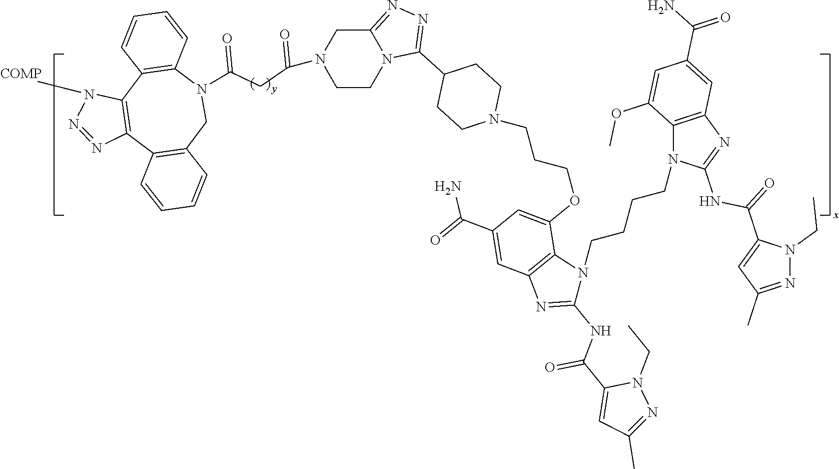

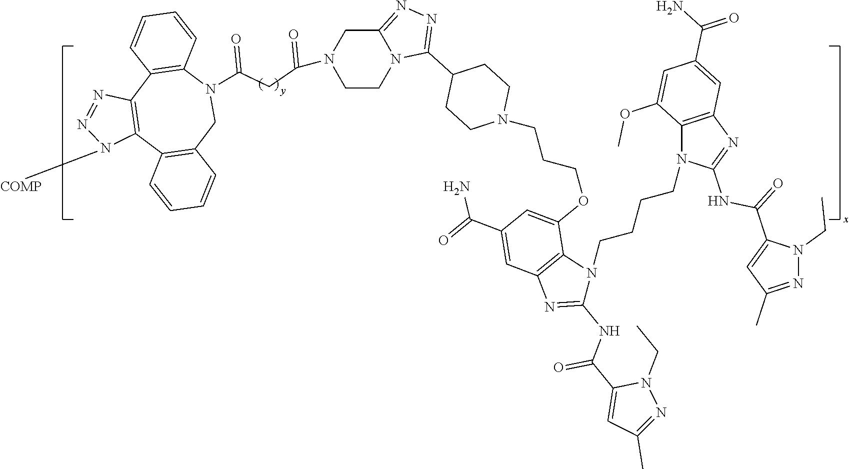

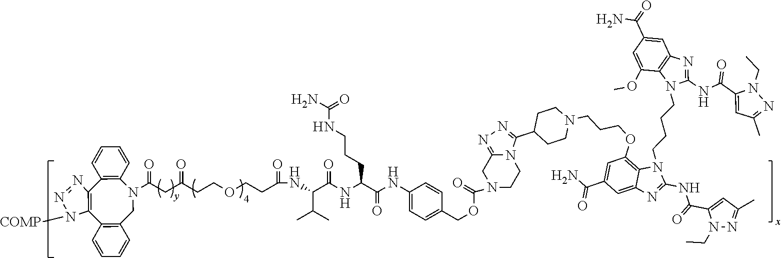

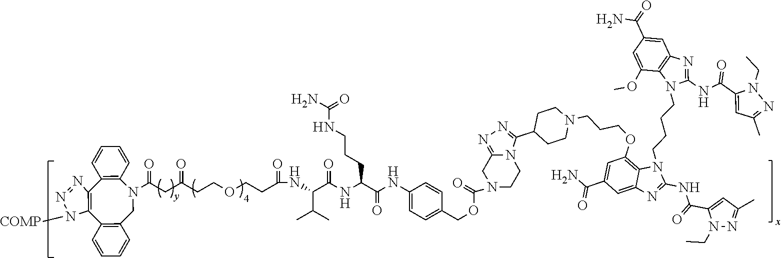

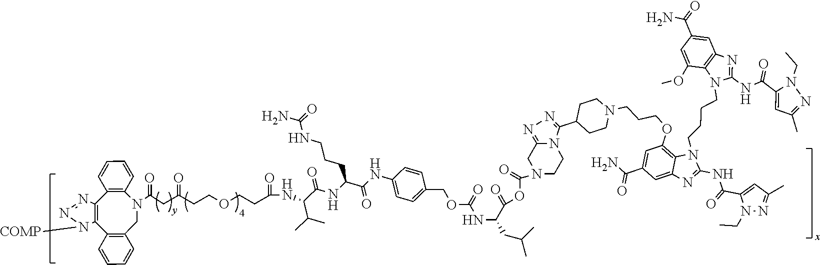

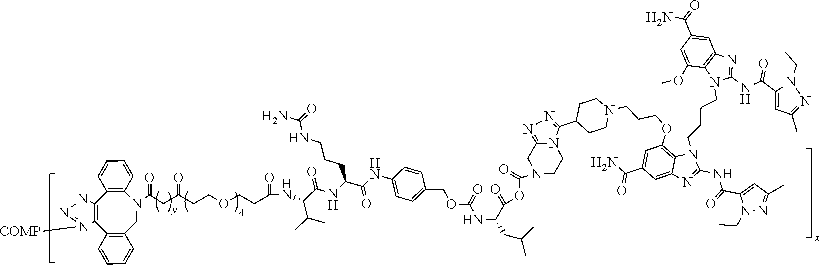

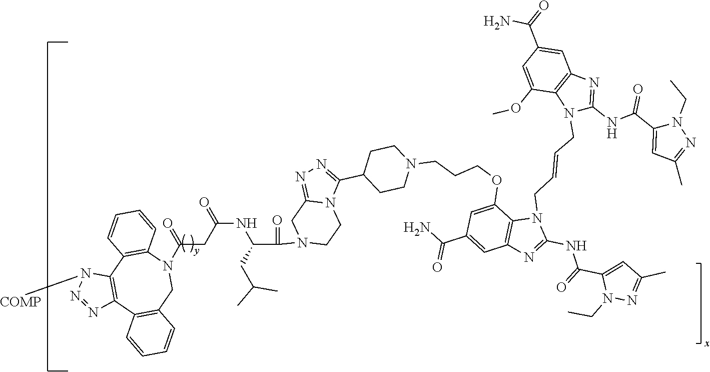

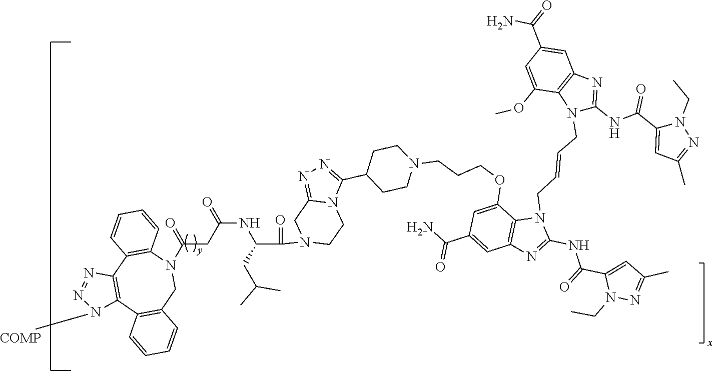

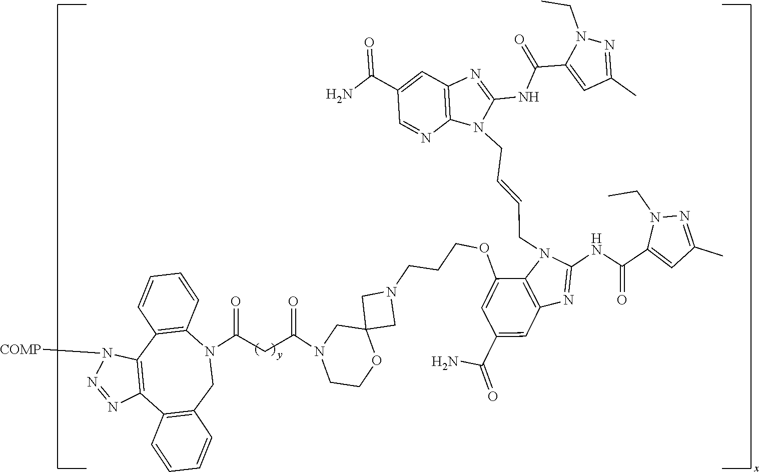

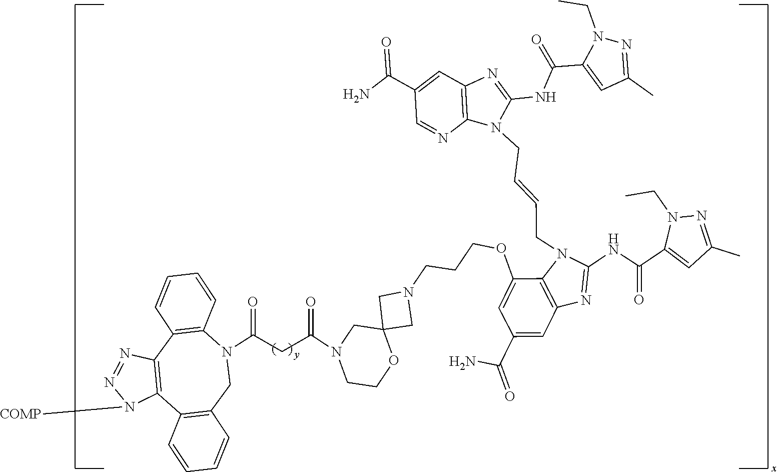

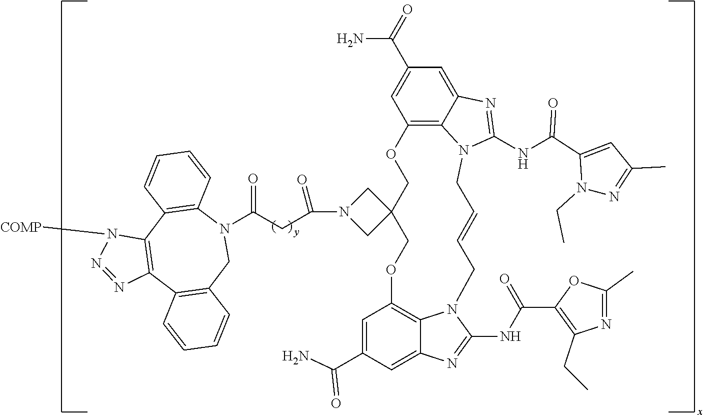

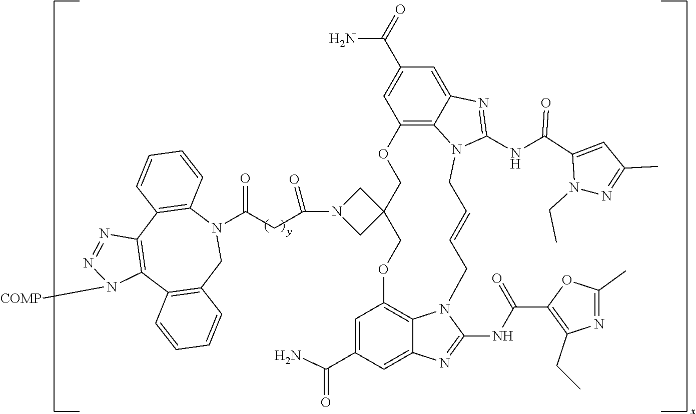

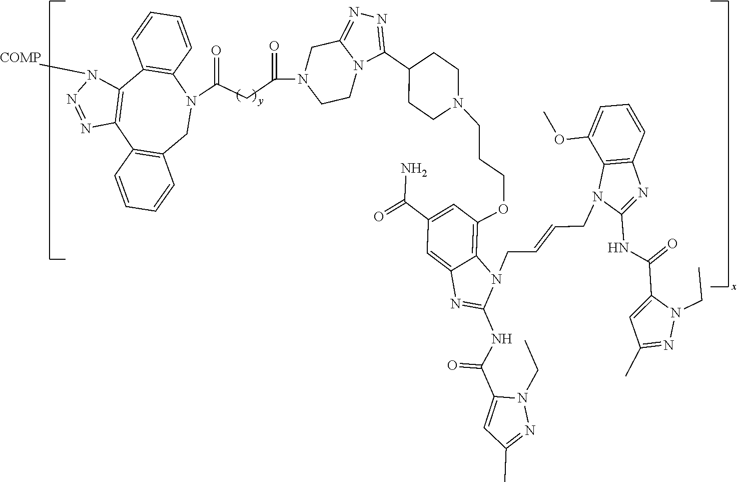

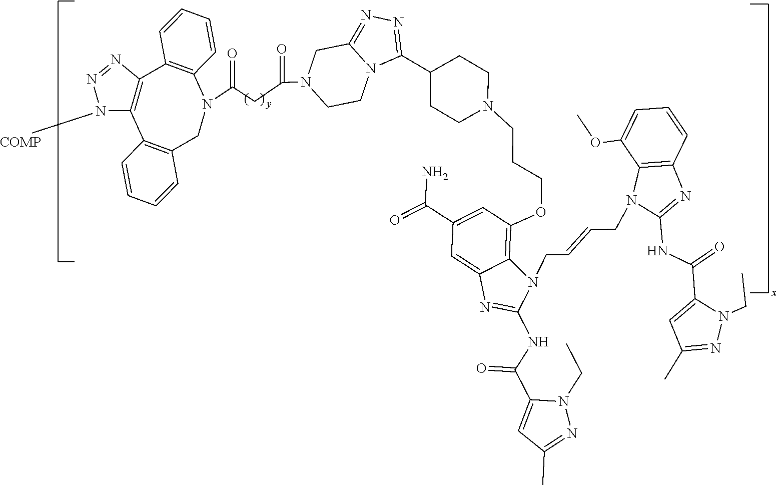

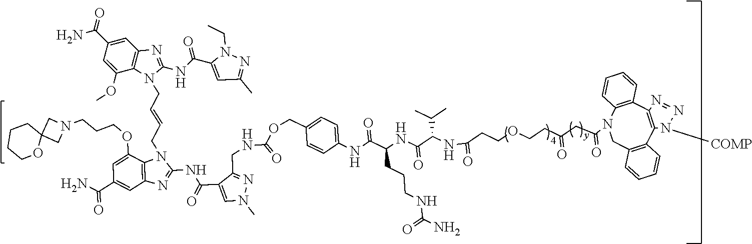

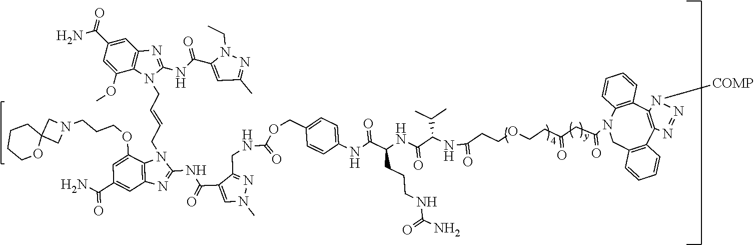

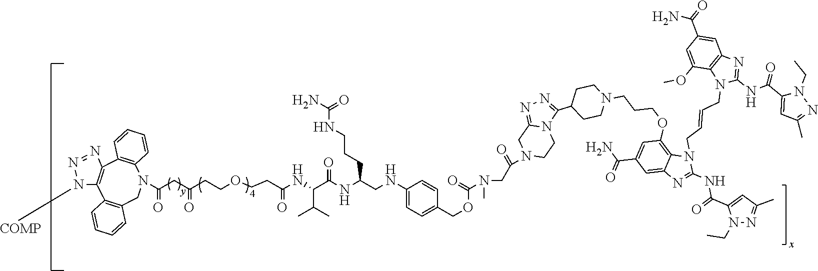

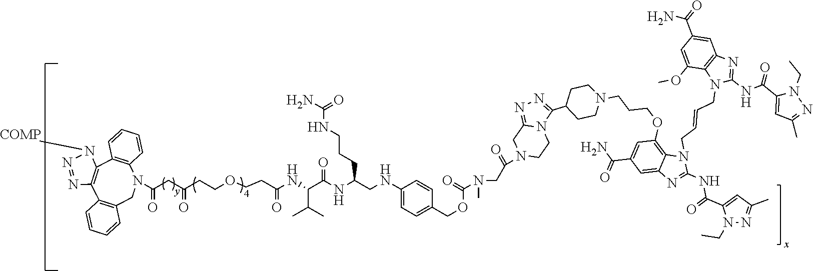

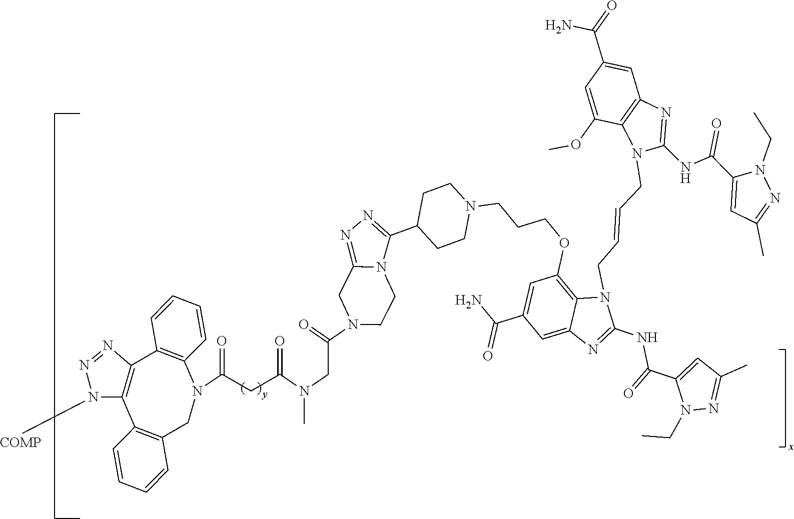

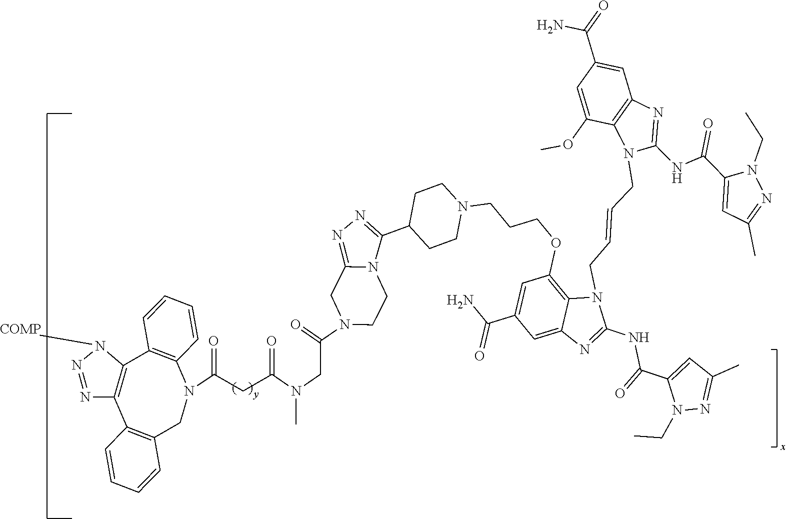

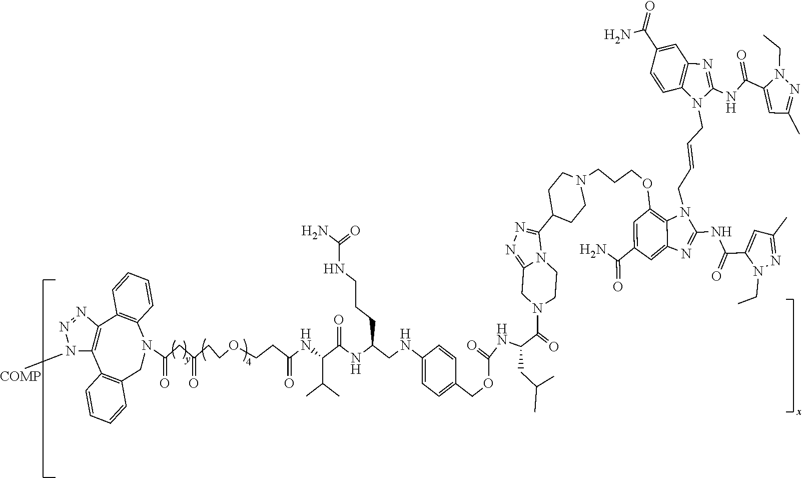

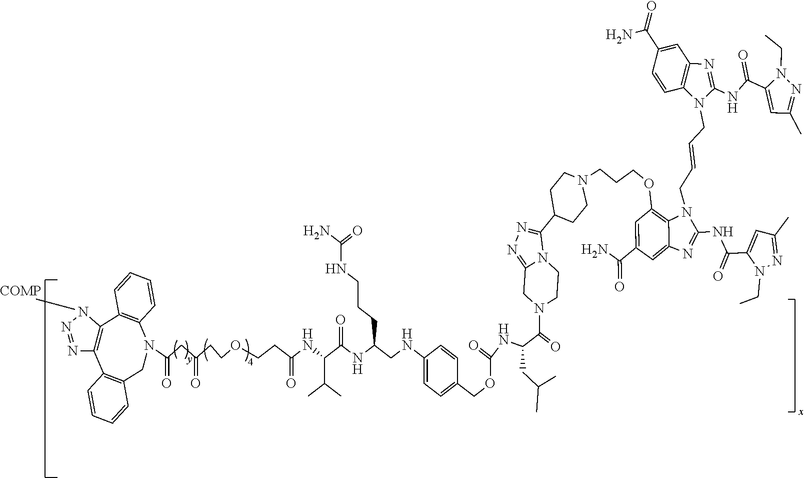

TABLE C-1

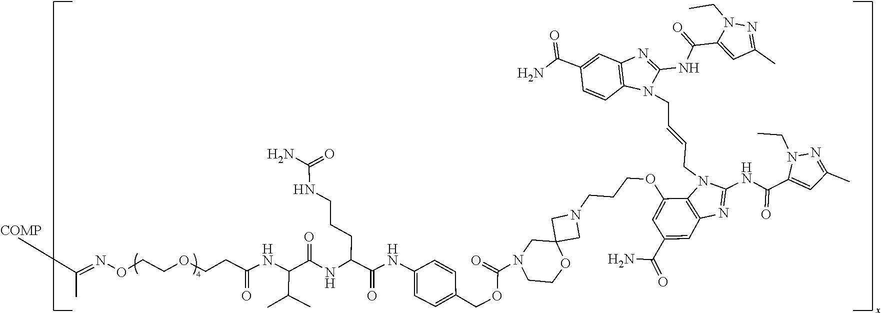

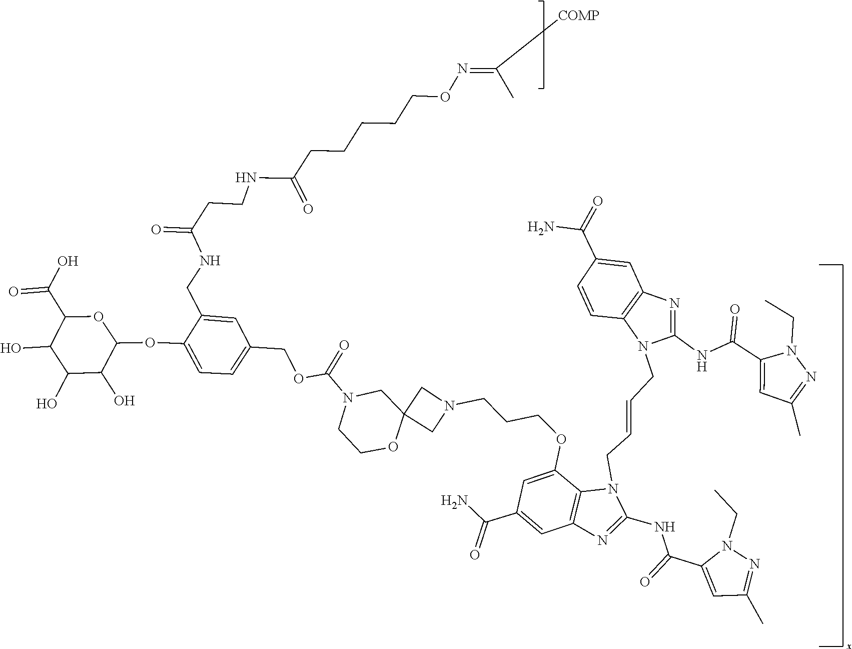

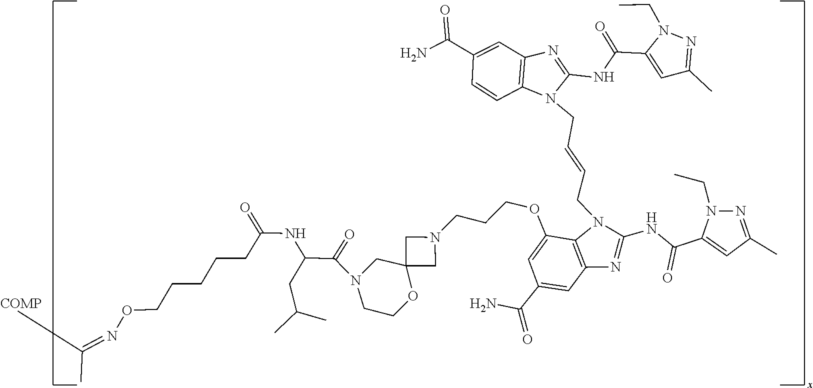

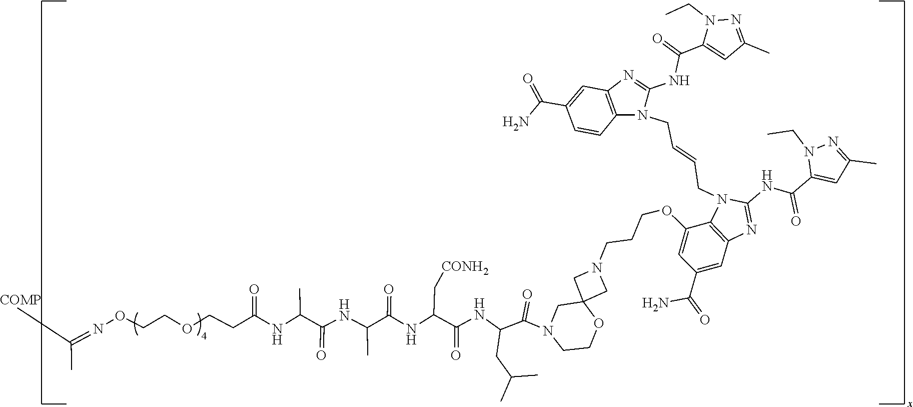

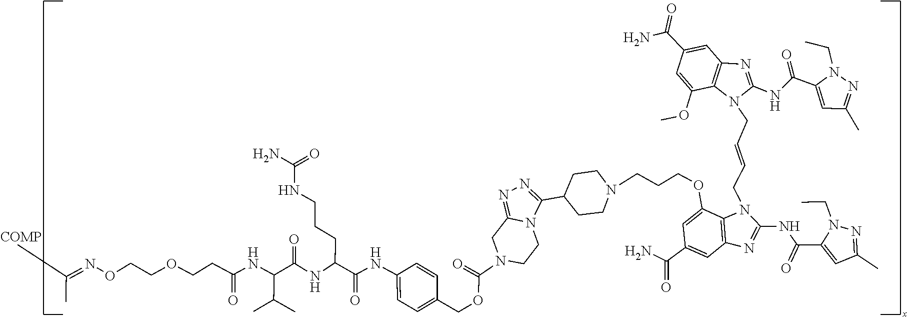

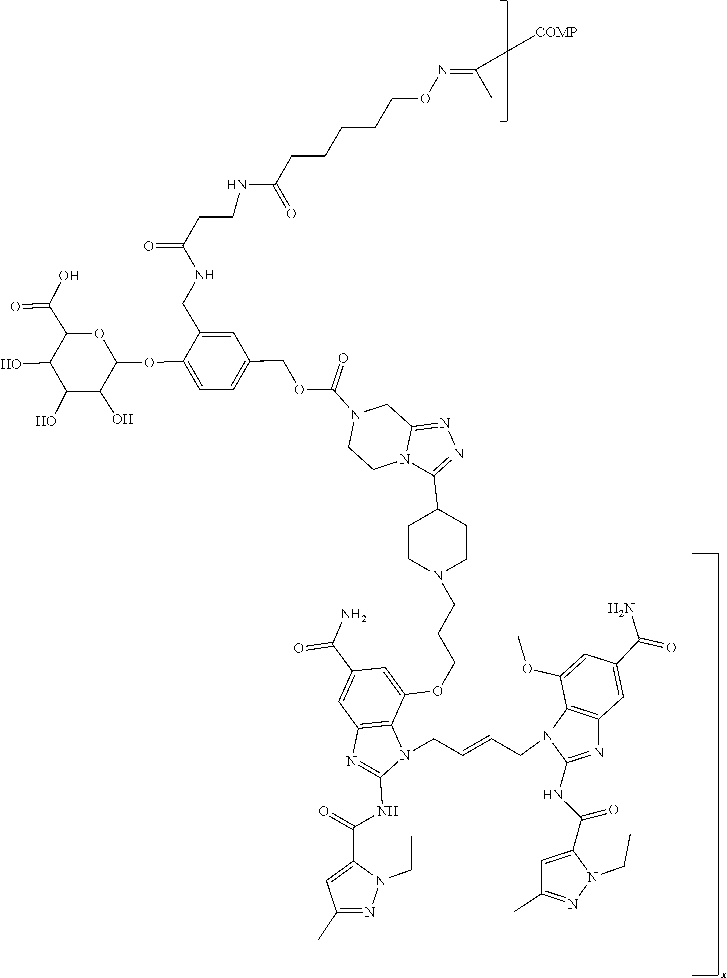

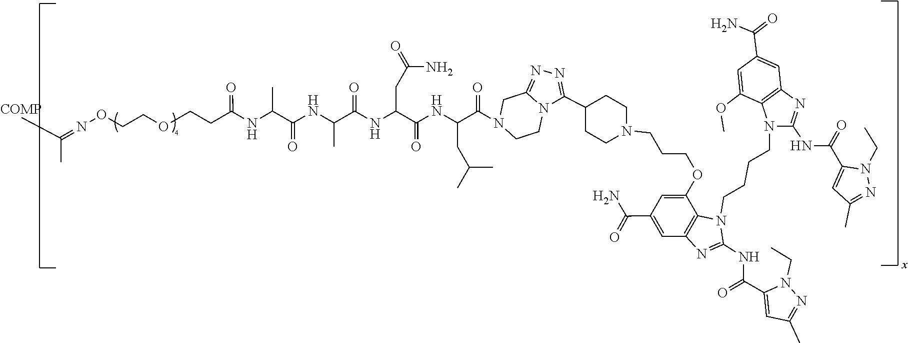

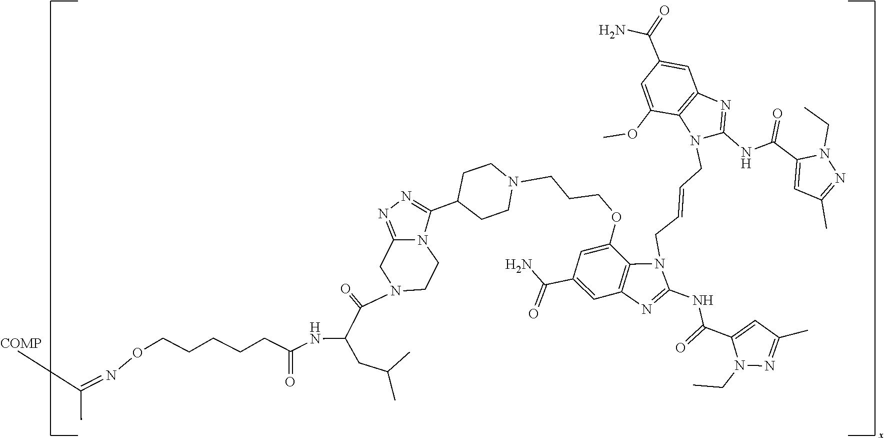

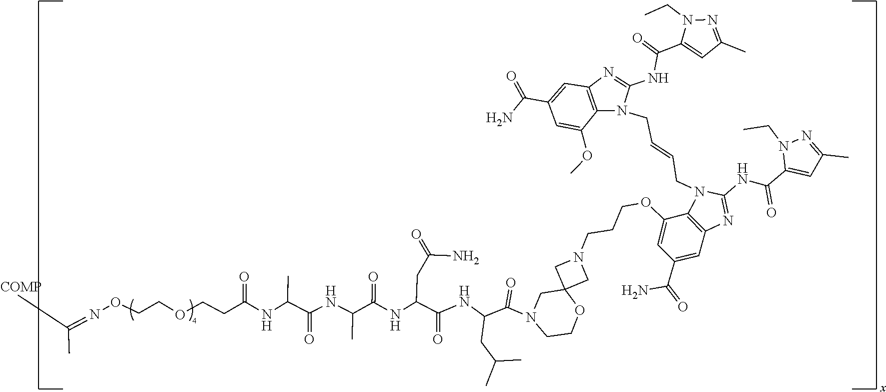

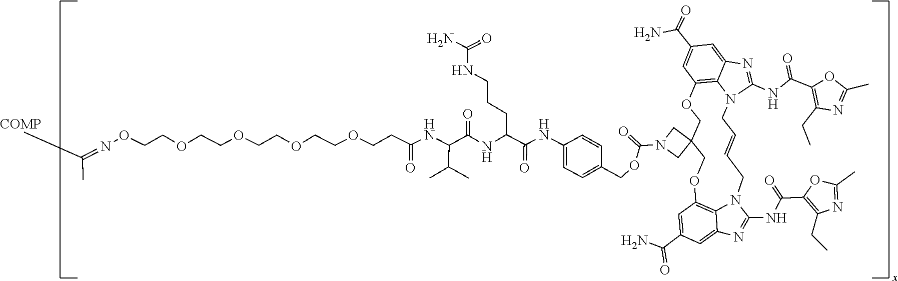

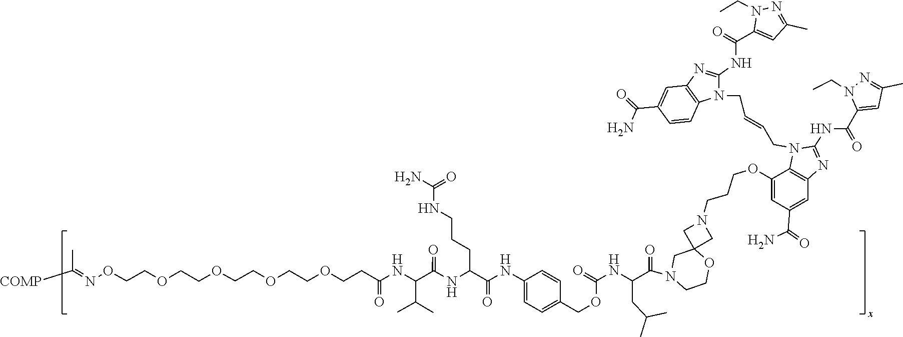

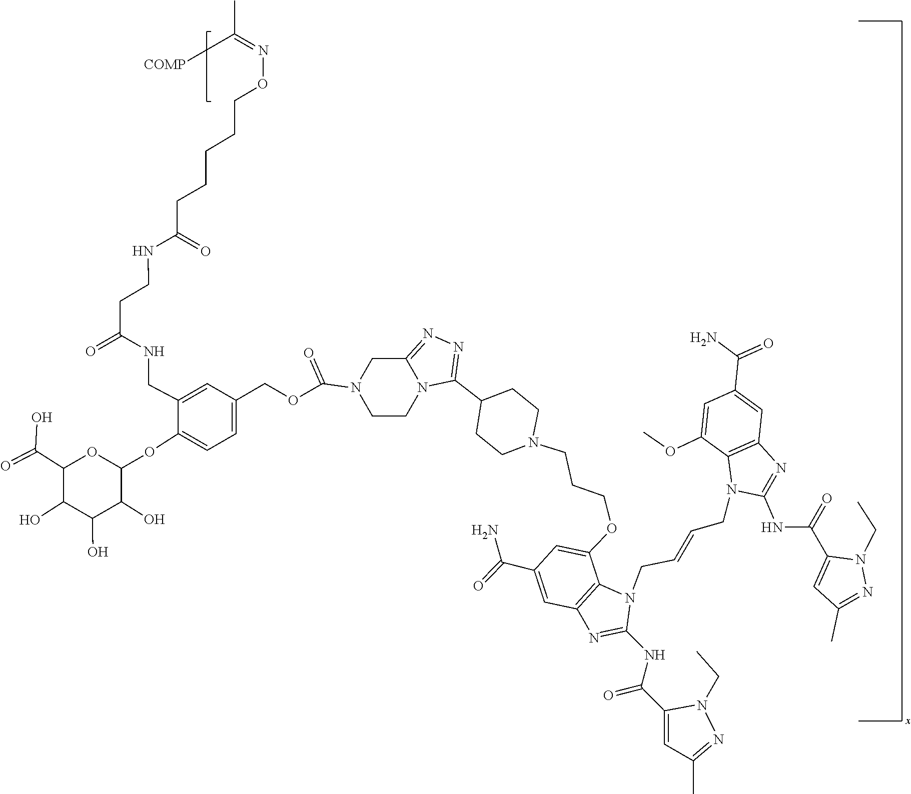

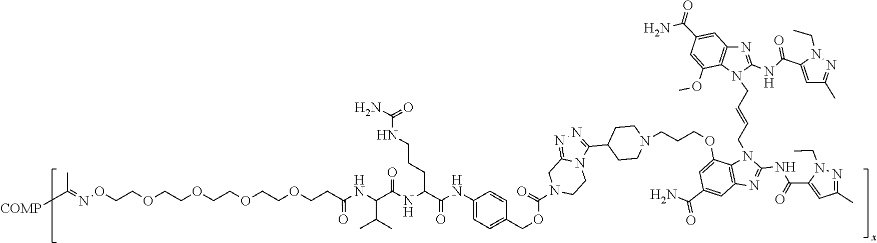

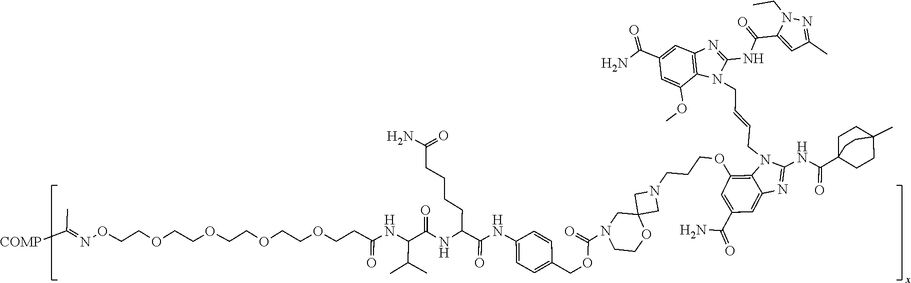

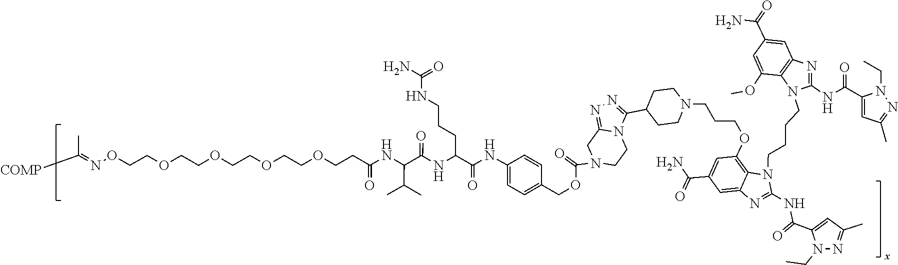

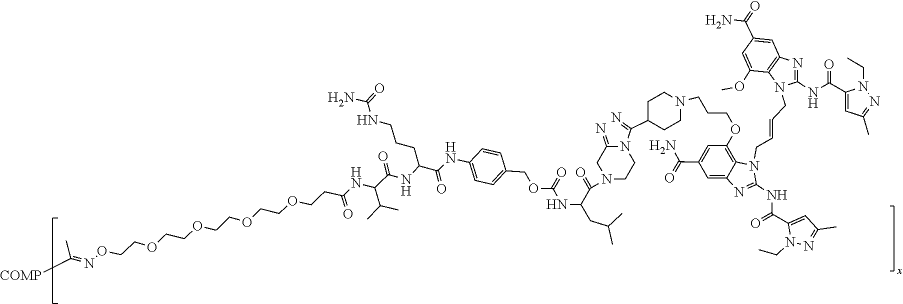

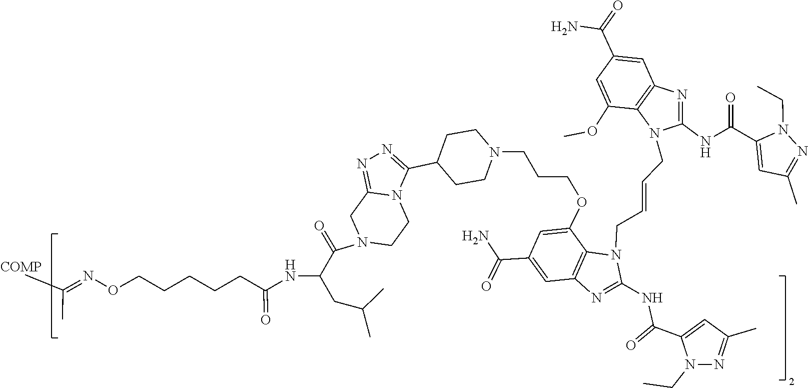

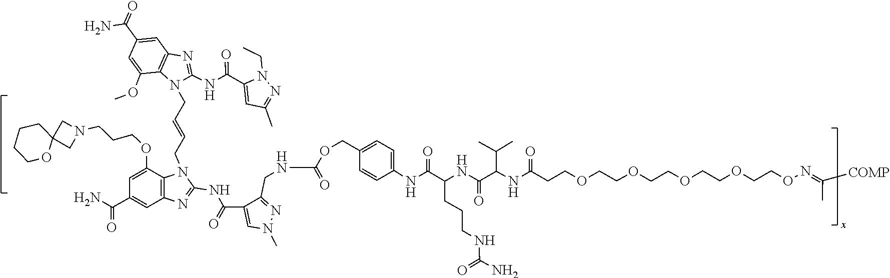

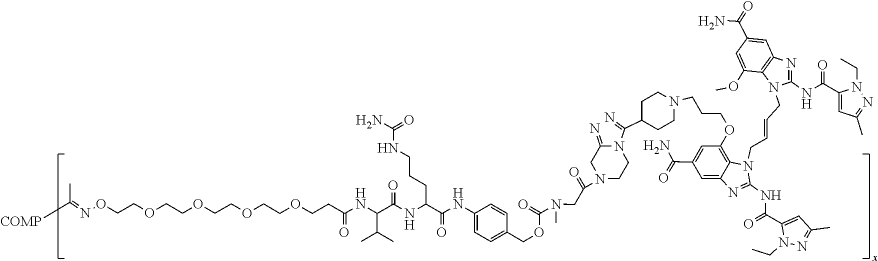

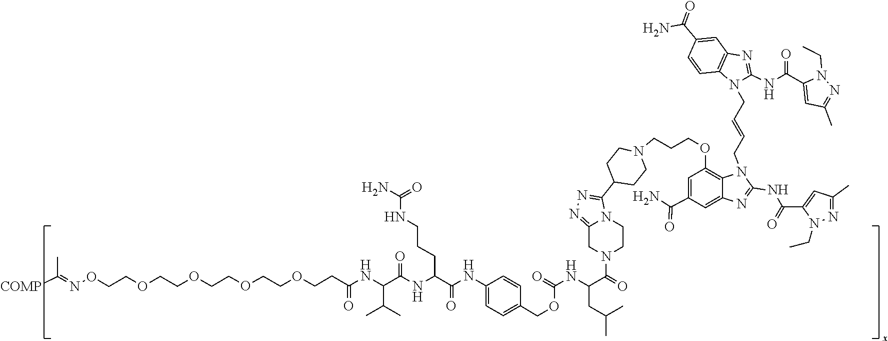

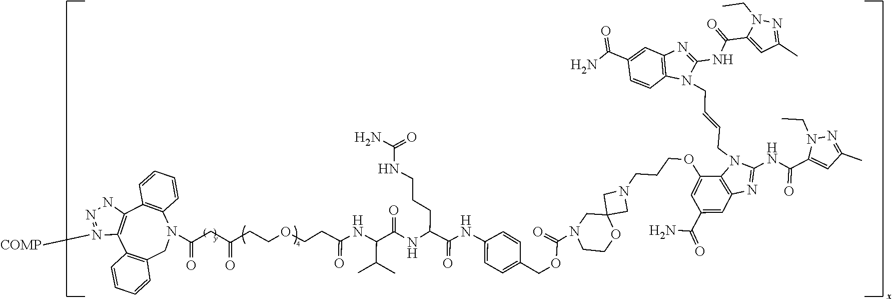

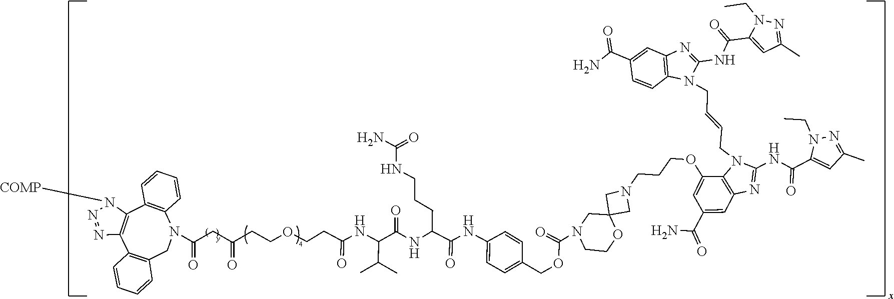

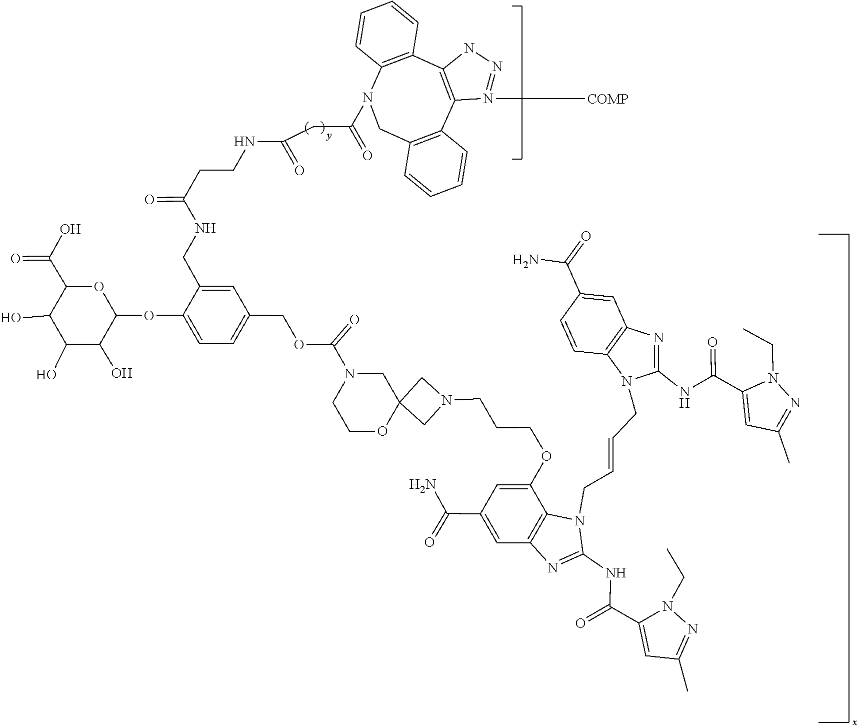

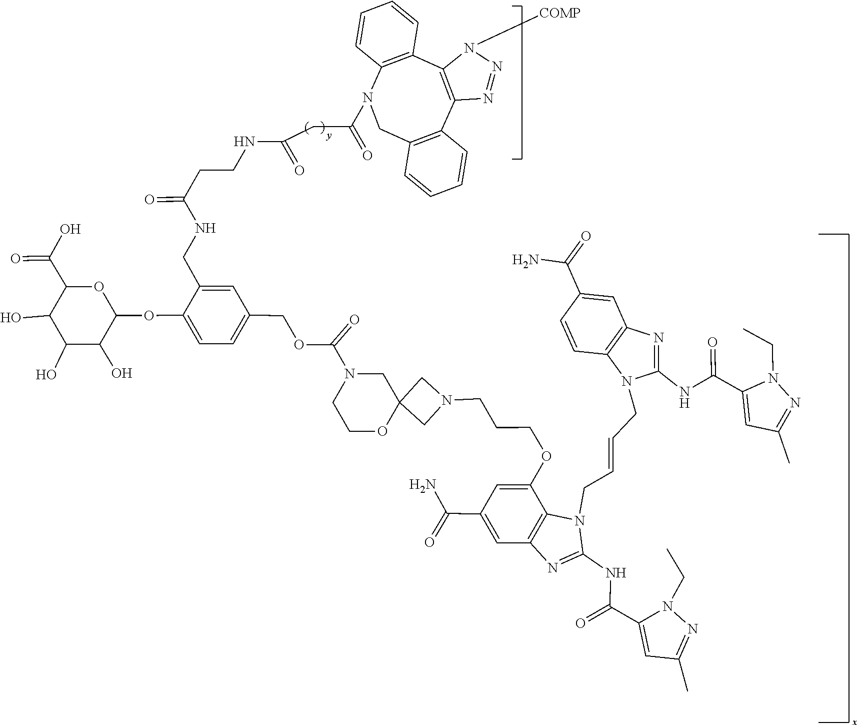

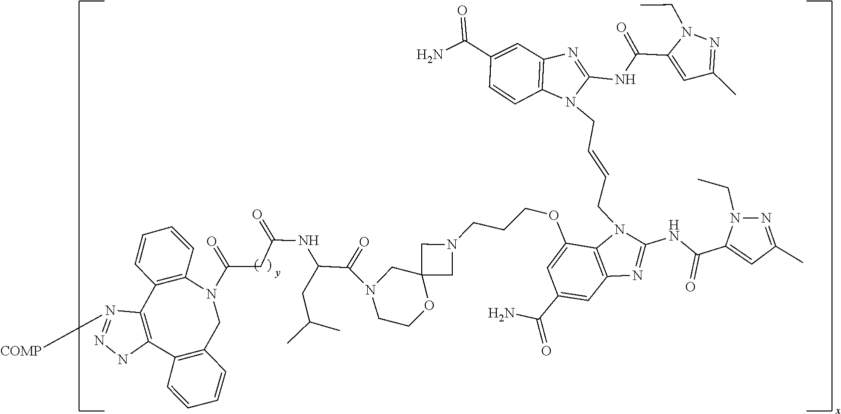

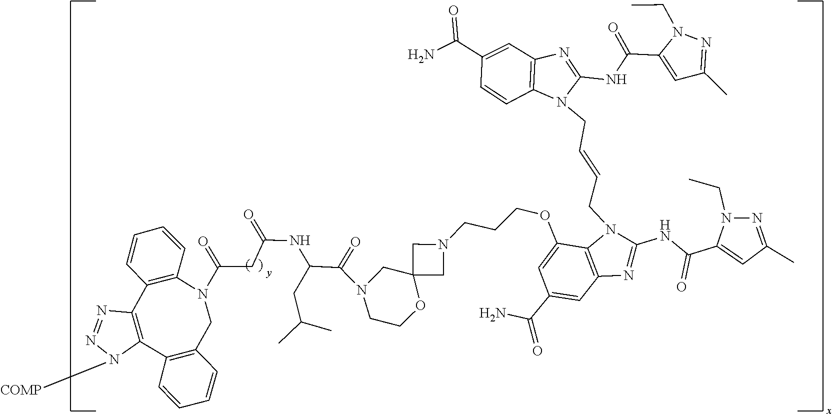

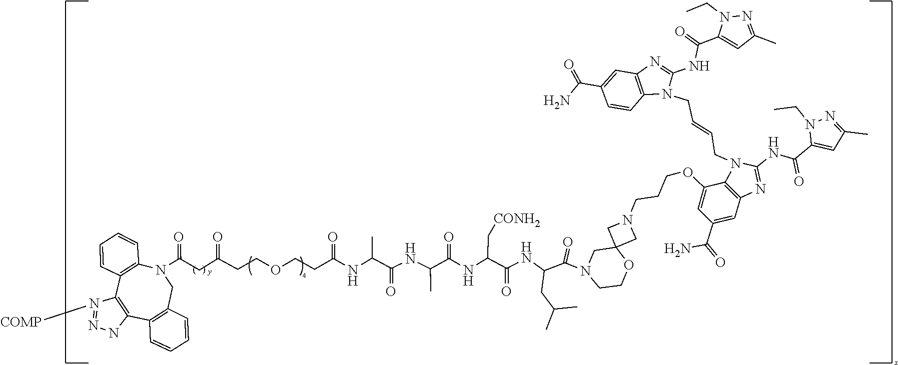

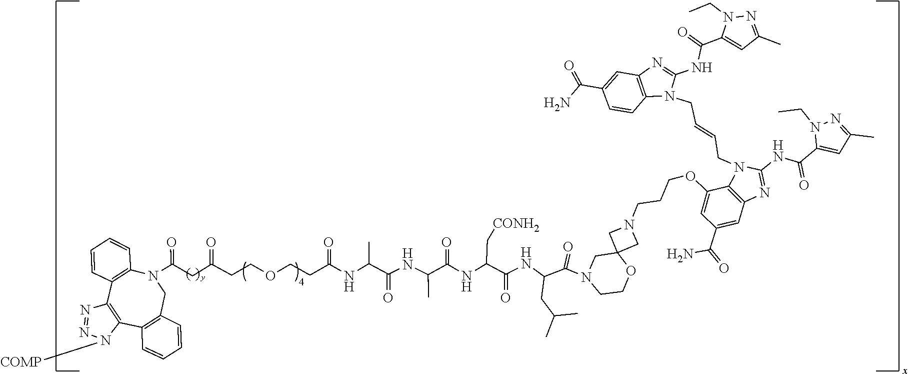

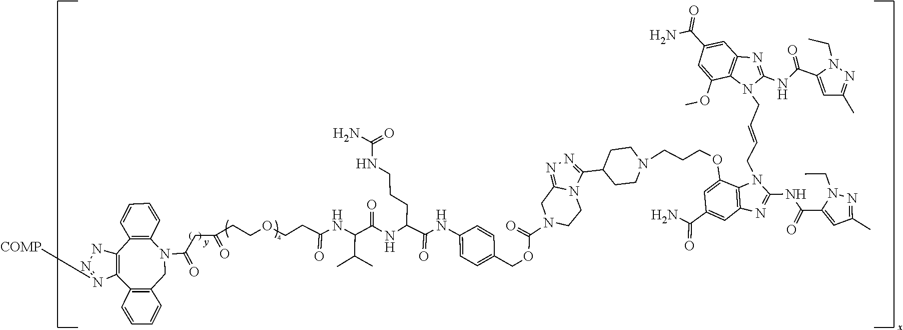

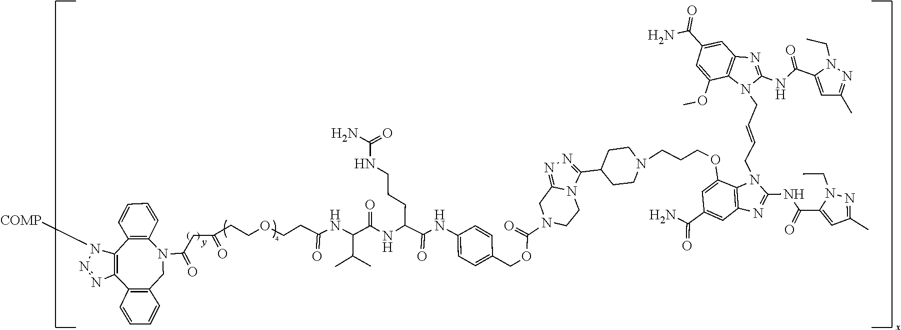

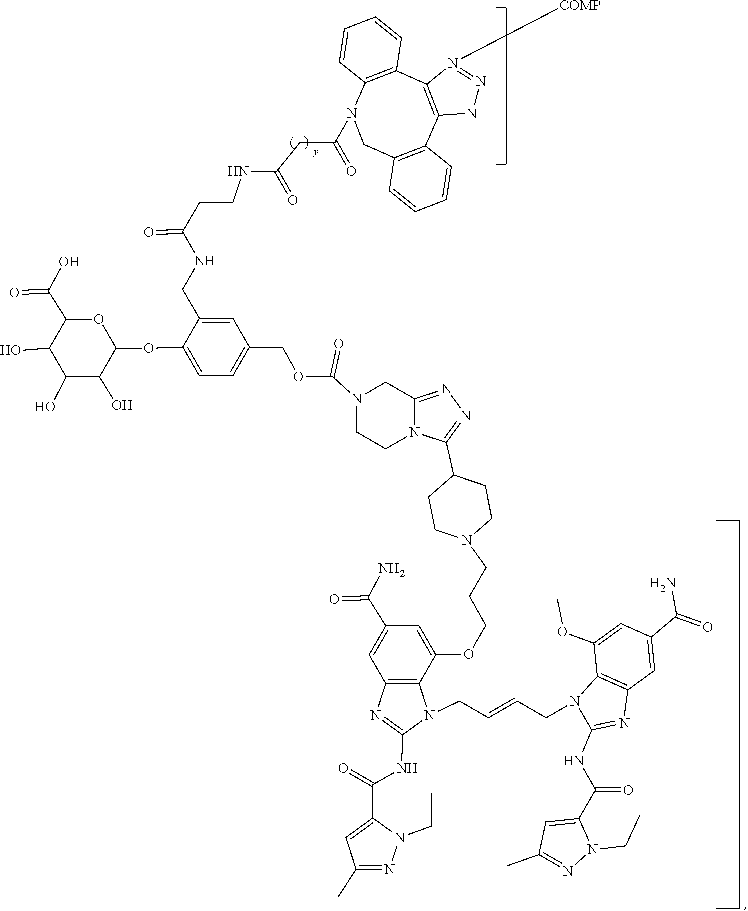

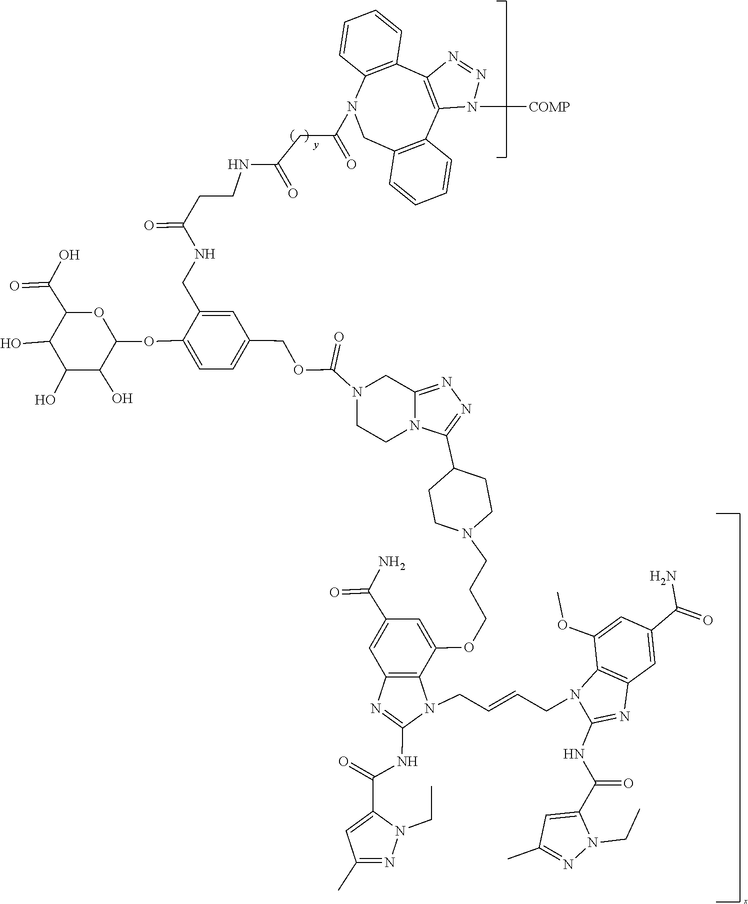

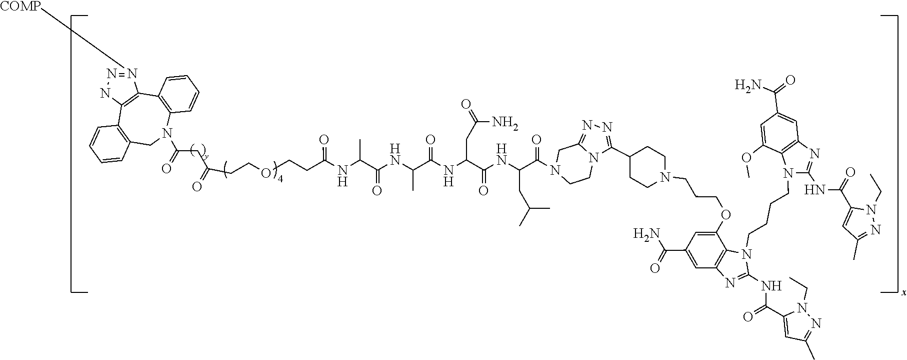

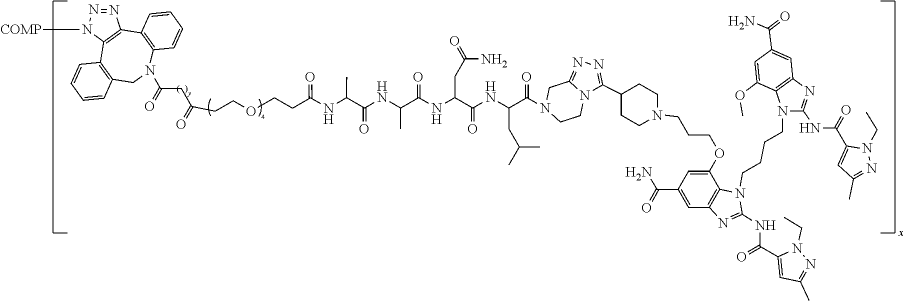

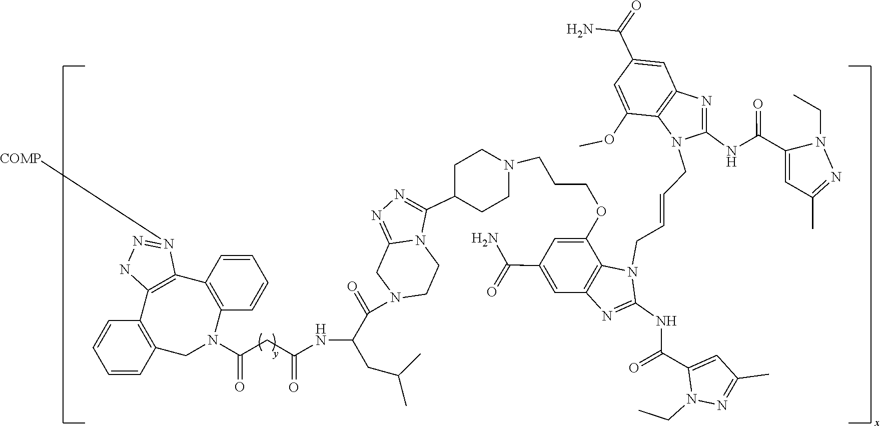

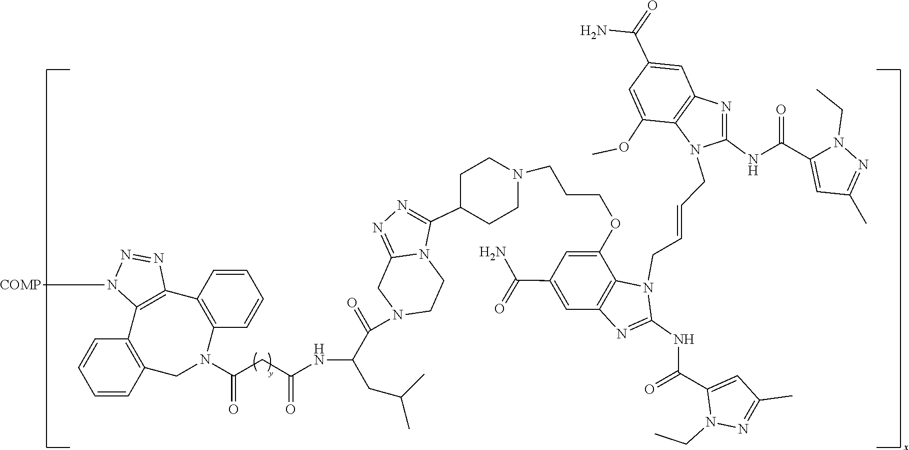

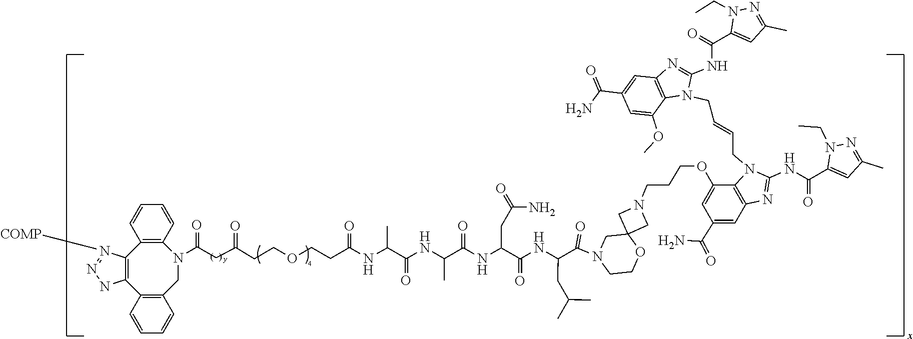

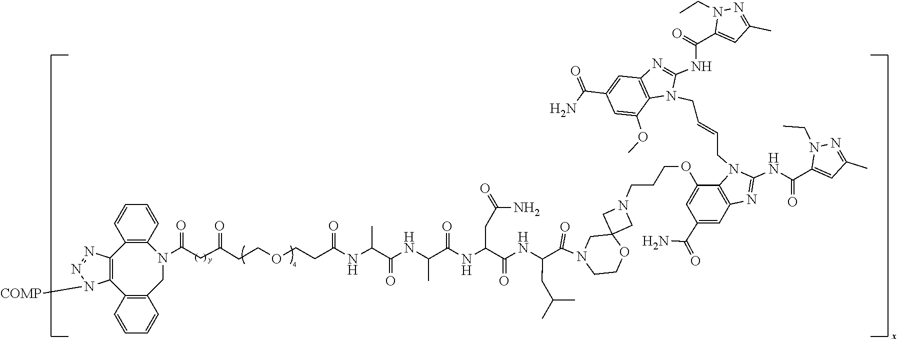

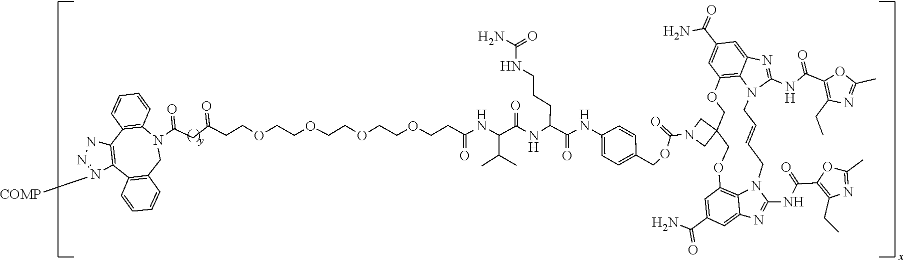

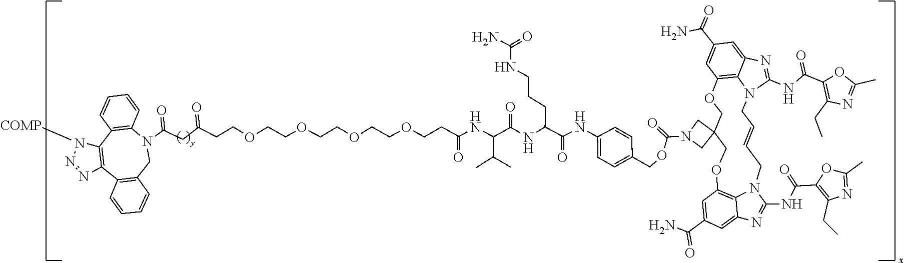

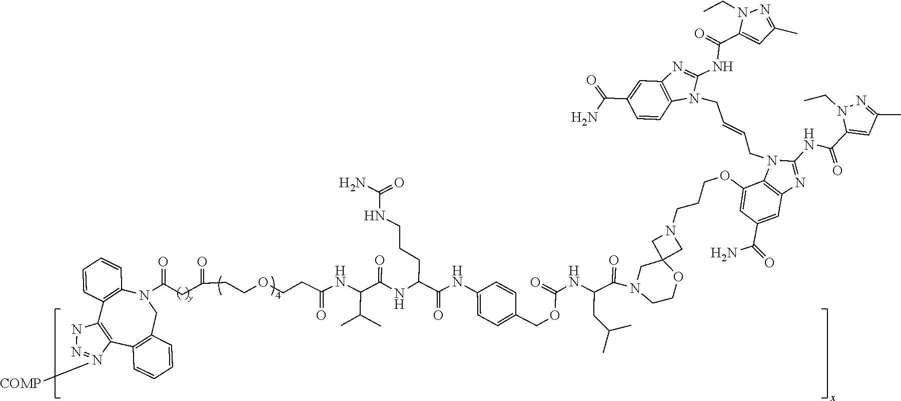

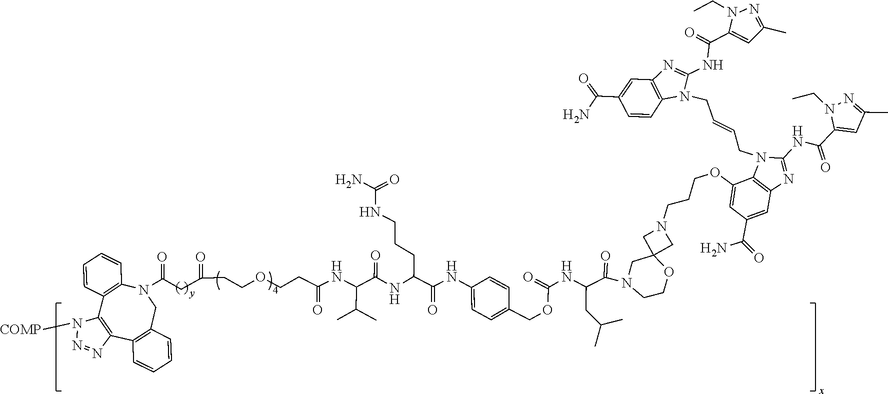

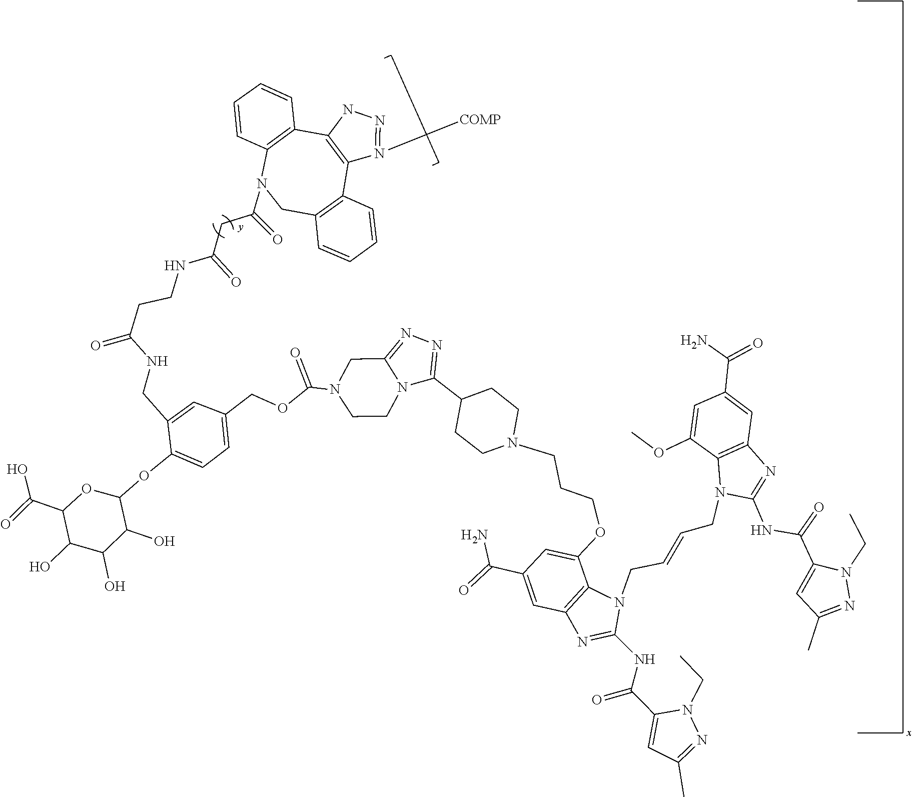

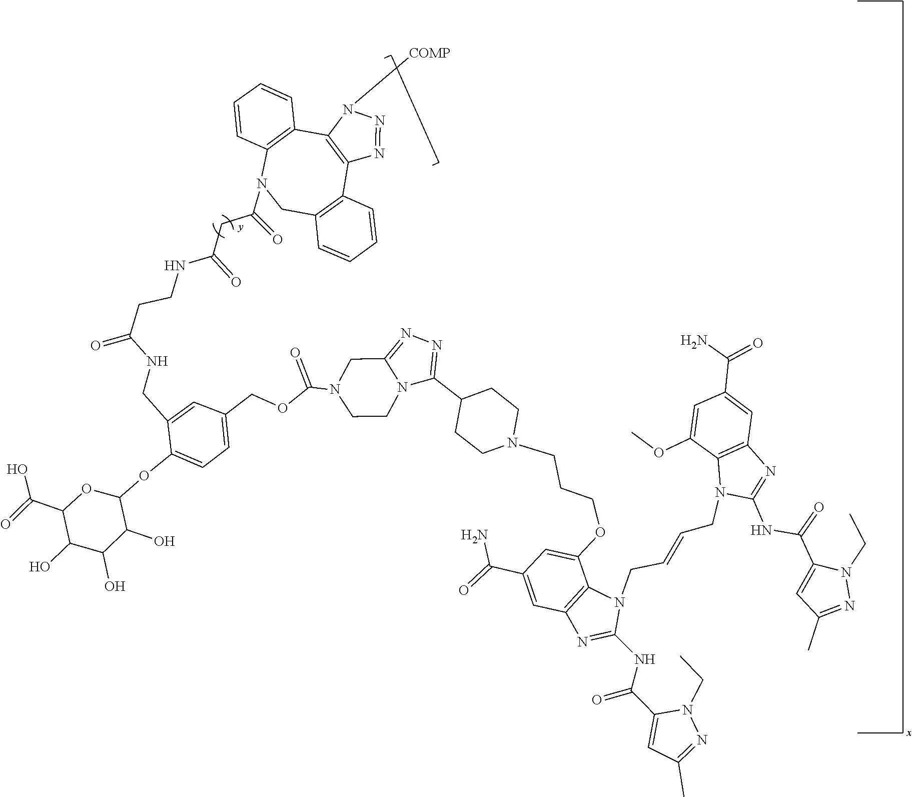

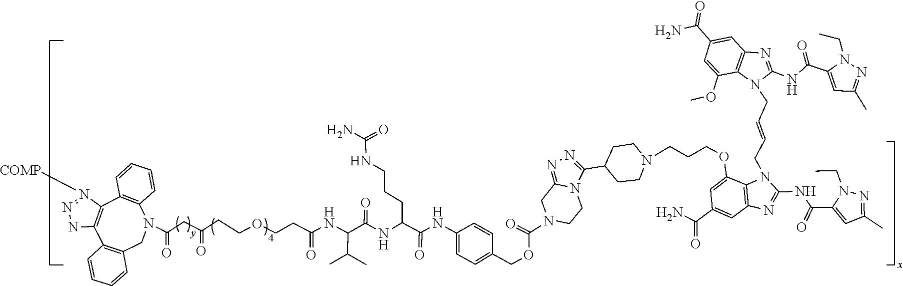

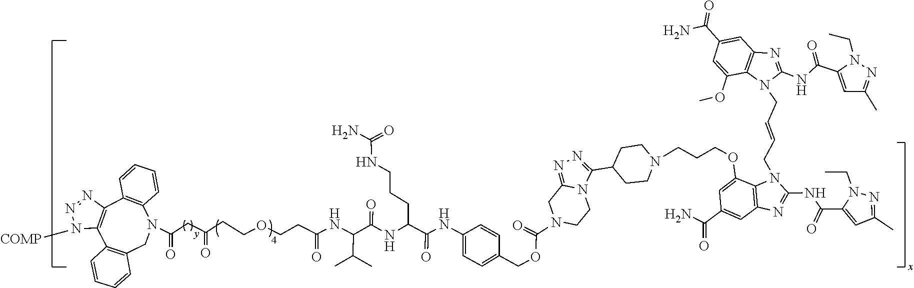

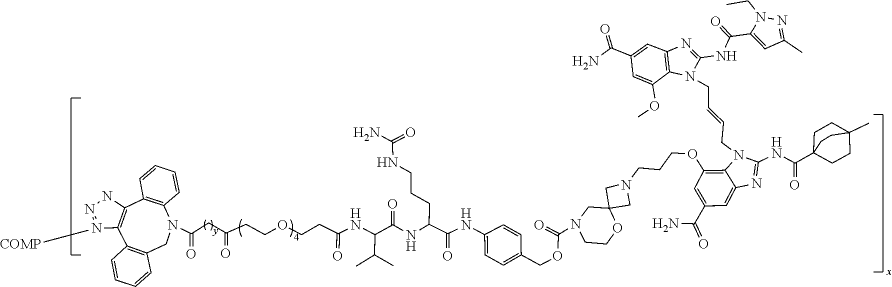

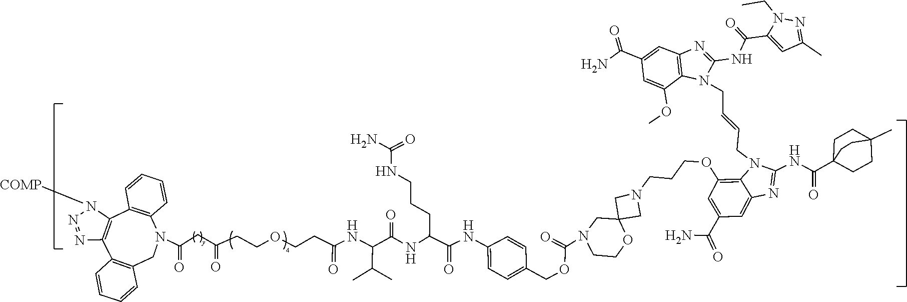

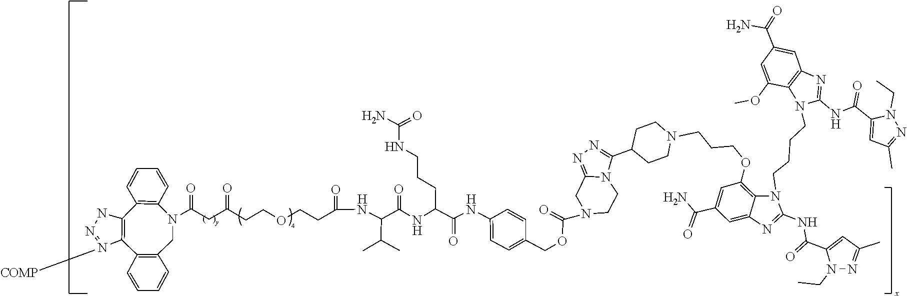

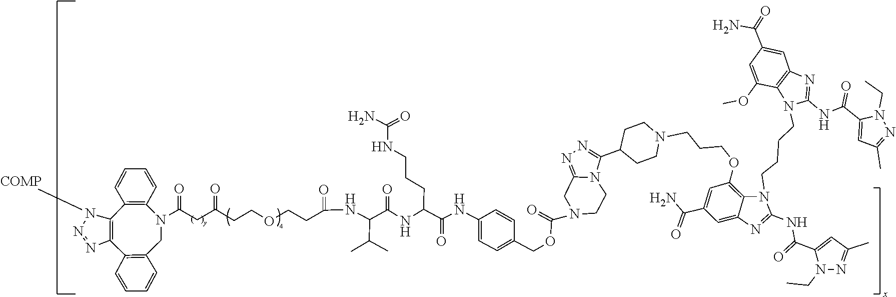

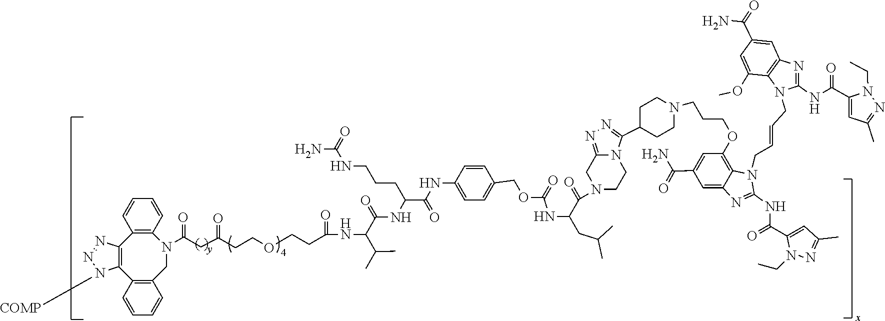

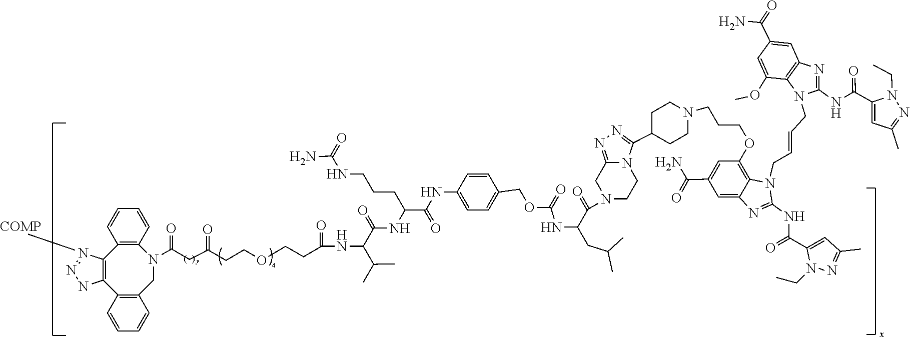

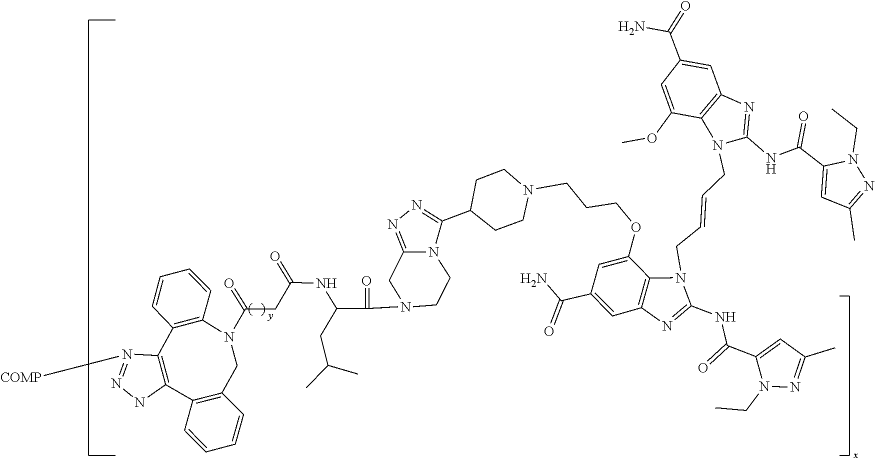

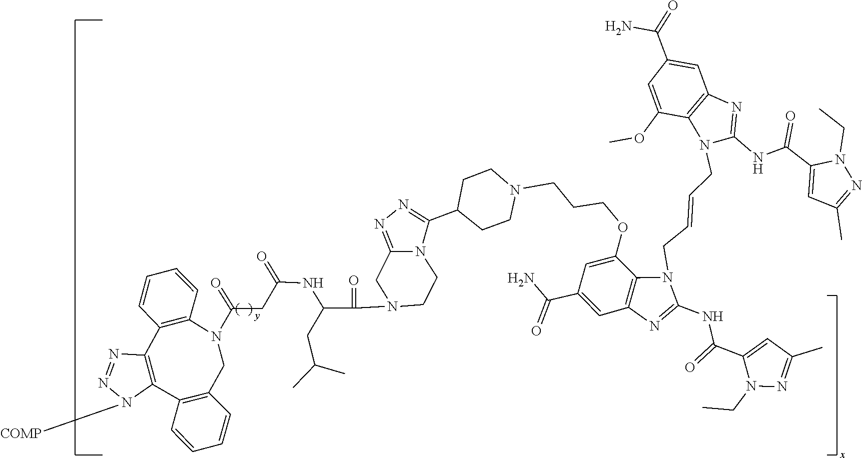

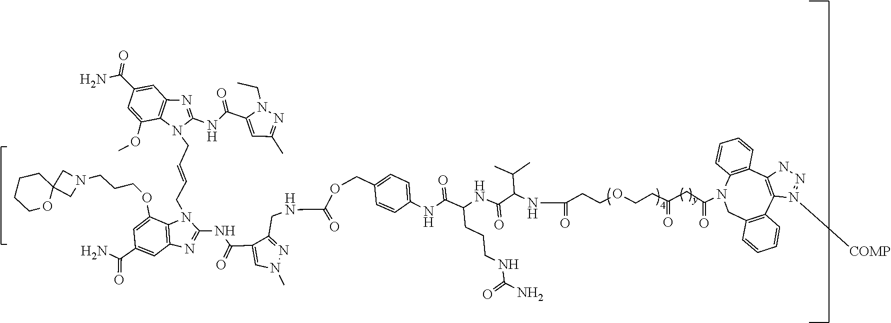

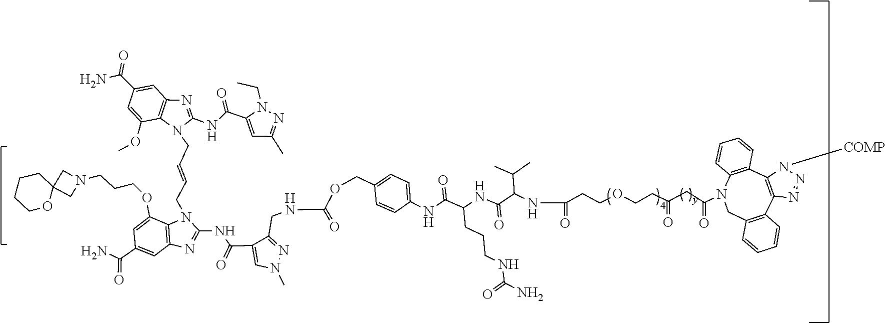

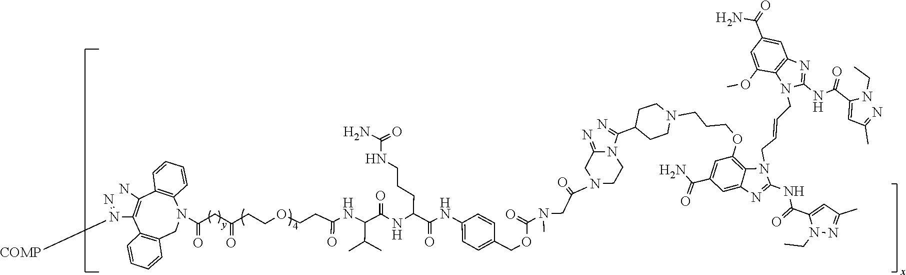

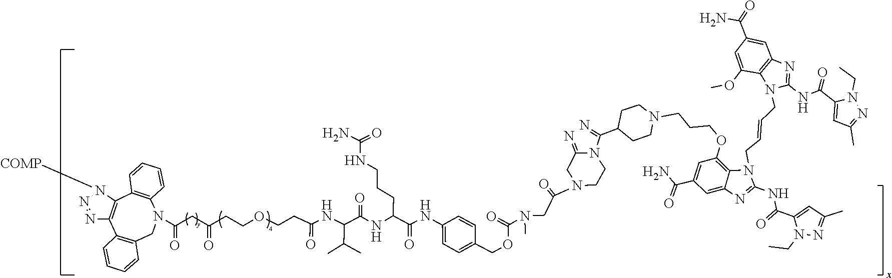

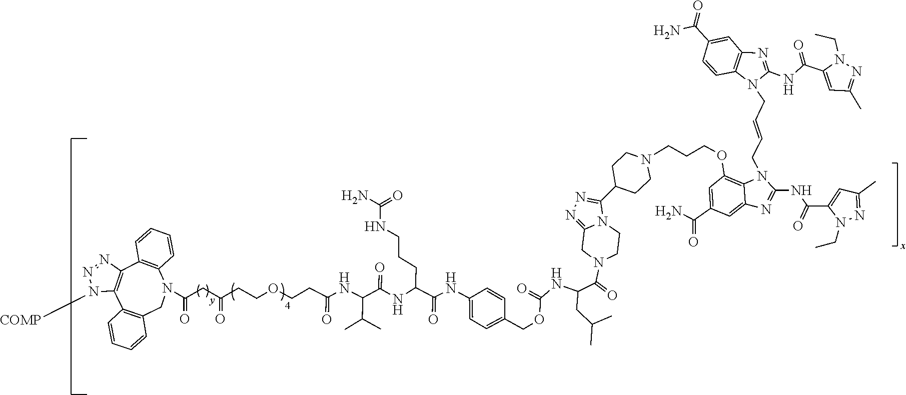

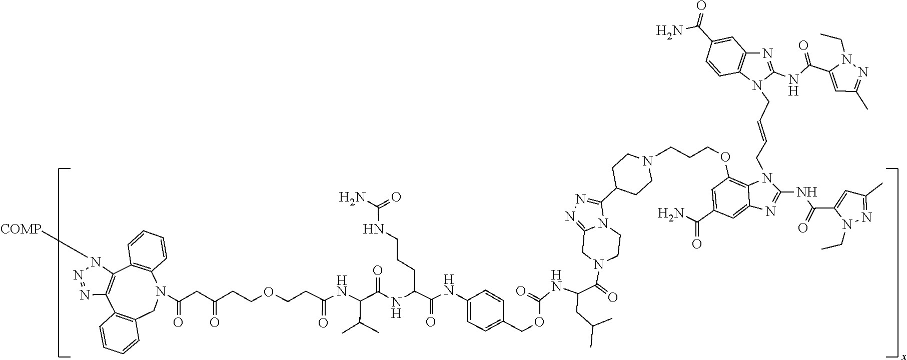

d. Optically Active Compounds

In certain embodiments, compounds, linker-payloads, and conjugates provided herein may have several chiral centers and may exist in and be isolated in optically active and racemic forms. In certain embodiments, some compounds, linker-payloads, or conjugates may exhibit polymorphism. A person of skill in the art will appreciate that compounds, linker-payloads, and conjugates provided herein can exist in any racemic, optically-active, diastereomeric, polymorphic, regioisomeric and/or stereoisomeric form, and/or mixtures thereof.

A person of skill in the art will also appreciate that such compounds, linker-payloads, and conjugates described herein that possess the useful properties also described herein are within the scope of this disclosure. A person of skill in the art will further appreciate how to prepare optically active forms of the compounds, linker-payloads, and conjugates described herein, for example, by resolution of racemic forms via recrystallization techniques, by synthesis from optically-active starting materials, by chiral synthesis, or by chromatographic separation using a chiral stationary phase. In addition, most amino acids are chiral (i.e., designated as L- or D-, wherein the L-enantiomer is the naturally occurring configuration) and can exist as separate enantiomers.

Examples of methods to obtain optically active materials are known in the art, and include at least the following:
i) physical separation of crystals—a technique whereby macroscopic crystals of the individual enantiomers are manually separated. This technique can be used if crystals of the separate enantiomers exist (i.e., the material is a conglomerate, and the crystals are visually distinct);
ii) simultaneous crystallization—a technique whereby the individual enantiomers are separately crystallized from a solution of the racemate, only if the latter is a conglomerate in the solid state;
iii) enzymatic resolutions—a technique wherein partial or complete separation of a racemate is accomplished by virtue of different rates of reaction of the enantiomers in the presence of an enzyme;
iv) enzymatic asymmetric synthesis—a synthetic technique wherein at least one step of the synthesis uses an enzymatic reaction to obtain an enantiomerically pure or enriched synthetic precursor of the desired enantiomer;
v) chemical asymmetric synthesis—a synthetic technique wherein the desired enantiomer is synthesized from an achiral precursor using chiral catalysts or chiral auxiliaries to produce asymmetry (i.e., chirality) in the product;
vi) diastereomer separations—a technique wherein a racemic compound is treated with an enantiomerically pure reagent (a chiral auxiliary) that converts the individual enantiomers to diastereomers. The resulting diastereomers are then separated by chromatography or crystallization by virtue of their now more distinct diastereomeric differences, and then the chiral auxiliary is removed to obtain each enantiomer;
vii) first- and second-order asymmetric transformations—a technique wherein diastereomers of the racemate equilibrate in solution to yield a preponderance of a diastereomer of the desired enantiomer, or where kinetic or thermodynamic crystallization of the diastereomer of the desired enantiomer perturbs the equilibrium such that eventually in principle all the material is converted to the crystalline diastereomer of the desired enantiomer. The desired enantiomer is then derived from the diastereomer;
viii) kinetic resolutions—this technique refers to the achievement of partial or complete resolution of a racemate (or of a further resolution of a partially resolved compound) by virtue of unequal reaction rates of the enantiomers with a chiral or non-racemic reagent or catalyst under kinetic conditions;
ix) enantiospecific synthesis from non-racemic precursors—a synthetic technique wherein the desired enantiomer is obtained from chiral starting materials and where the stereochemical integrity is not or is only minimally compromised over the course of the synthesis;
x) chiral liquid chromatography—a technique wherein the enantiomers of a racemate are separated in a liquid mobile phase by virtue of their different interactions with a stationary phase. The stationary phase can be made of chiral material or the mobile phase can contain an additional chiral material to provoke the different interactions;
xi) chiral gas chromatography—a technique wherein the racemate is volatilized and enantiomers are separated by virtue of their different interactions in the gaseous mobile phase with a column containing a fixed non-racemic adsorbent phase;
xii) extraction with chiral solvents—a technique wherein the enantiomers are separated by virtue of kinetic or thermodynamic dissolution of one enantiomer into a particular chiral solvent;
a) transport across chiral membranes—a technique wherein a racemate is placed in contact with a thin membrane barrier. The barrier typically separates two miscible fluids, one containing the racemate, and a driving force such as a concentration or pressure differential causes preferential transport across the membrane barrier. Separation occurs as a result of the non-racemic nature of the membrane which allows only one enantiomer of the racemate to pass through.

In some embodiments, provided herein are compositions of the compounds, linker-payloads, or conjugates of the present disclosure that are substantially free of a designated stereoisomer of that compound, linker-payload, or conjugate, respectively. In certain embodiments, in the methods, compounds, linker-payloads, and conjugates of this disclosure, the compounds, linker-payloads, or conjugates are substantially free of other stereoisomers. In some embodiments, the composition includes a compound, linker-payload, or conjugate that is at least 85%, 90%, 95%, 98%, or 99% to 100% by weight of the compound, linker-payload or conjugate, respectively, the remainder comprising other chemical species or enantiomers. In some embodiments, provided herein are compositions of compounds of Formula (I)—(IV), linker-payloads of Formula (LP-1)-(LP-V), and conjugates of Formula (CONJ-I)—(CONJ-V) that are substantially free of a designated enantiomer of that compound, linker-payload, or conjugate, respectively. In certain embodiments, in the methods, compounds, linker-payloads, and conjugates of this disclosure, the compounds, linker-payloads, or conjugates are substantially free of other enantiomers. In some embodiments, the composition includes a compound, linker-payload, or conjugate that is at least 85%, 90%, 95%, 98%, or 99% to 100% by weight of the compound, linker-payload, or conjugate, respectively, the remainder comprising other chemical species or enantiomers.

e. Isotopically Enriched Compounds

Also provided herein are isotopically enriched compounds, linker-payloads, and conjugates including, but not limited to, isotopically enriched compounds of Formula (I)—(IV), linker payloads of Formula (LP-I)-(LPV), and conjugates of Formula (CONJ-I)—(CONJ-V).

Isotopic enrichment (for example, deuteration) of pharmaceuticals to improve pharmacokinetics (“PK”), pharmacodynamics (“PD”), and/or toxicity profiles, has been previously demonstrated within some classes of drugs. See, for example, Lijinsky et al., Food Cosmet. Toxicol., 20: 393 (1982); Lijinsky et al., J. Nat. Cancer Inst., 69: 1127 (1982); Mangold et al., Mutation Res. 308: 33 (1994); Gordon et al., Drug Metab. Dispos., 15: 589 (1987); Zello et al., Metabolism, 43: 487 (1994); Gately et al., J. Nucl. Med., 27: 388 (1986); Wade D, Chem. Biol. Interact. 117: 191 (1999).

Isotopic enrichment of a drug can be used, for example, to (1) reduce or eliminate unwanted metabolites; (2) increase the half-life of the parent drug; (3) decrease the number of doses needed to achieve a desired effect; (4) decrease the amount of a dose necessary to achieve a desired effect; (5) increase the formation of active metabolites, if any are formed; and/or (6) decrease the production of deleterious metabolites in specific tissues. Isotopic enrichment of a drug can also be used to create a more effective and/or safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e., the step with the highest transition state energy), substitution of a (heavier) isotope for that reactive hydrogen will cause a decrease in the reaction rate. The Deuterium Kinetic Isotope Effect (“DKIE”) is the most common form of KIE. (See, e.g., Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen and the C-D bond is broken. The DKIE can range from about one (no isotope effect) to very large numbers, such as 50 or more, meaning that the reaction can be fifty, or more, times slower when deuterium has been substituted for hydrogen.

Substitution of tritium (“T”) for hydrogen results in yet a stronger bond than deuterium and gives numerically larger isotope effects. Similarly, substitution of isotopes for other elements including, but not limited to, ¹³C or ¹⁴C for carbon; ³³S, ³⁴S, or ³⁶S for sulfur; ¹⁵N for nitrogen; and ¹⁷O or ¹⁸O for oxygen may lead to a similar kinetic isotope effect.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C=C) pi-bond. The resultant metabolites may be stable or unstable under physiological conditions, and can have substantially different PK/PD, and acute and long-term toxicity profiles relative to the parent compounds. For many drugs, such oxidations are rapid. Therefore, these drugs often require the administration of multiple or high daily doses.

Therefore, isotopic enrichment at certain positions of a compound provided herein will produce a detectable KIE that will affect the pharmacologic, PK, PD, and/or toxicological profiles of a compound provided herein in comparison with a similar compound having a natural isotopic composition.

### III. Conjugation

In certain embodiments, the conjugate can be formed from a macromolecule that comprises one or more reactive groups. In certain embodiments, the conjugate can be formed from a macromolecule comprising all naturally encoded amino acids. Those of skill in the art will recognize that several naturally encoded amino acids include reactive groups capable of conjugation to a compound of Formula (I)—(IV) or to a linker-payload of Formula (LP-I)-(LP-V). These reactive groups include cysteine side chains, lysine side chains, and amino-terminal groups. In these embodiments, the conjugate can comprise a compound of Formula (I)—(IV) or linker-payload of Formula (LP-I)-(LP-V) linked to the residue of an antibody reactive group. In these embodiments, the compound of Formula (I)—(IV) precursor or linker-payload of Formula (LP-I)-(LP-V) precursor comprises a reactive group capable of forming a bond with an antibody or antigen binding fragment thereof reactive group. Typical reactive groups include maleimide groups, activated carbonates (including, but not limited to, p-nitrophenyl ester), activated esters (including, but not limited to, N-hydroxysuccinimide, p-nitrophenyl ester, and aldehydes). Particularly useful reactive groups include maleimide and succinimide, for instance N-hydroxysuccinimide, for forming bonds to cysteine and lysine side chains. Further reactive groups are described in the sections and examples below.

### Reactive Groups

Reactive groups facilitate conjugation of the compounds of Formula (I)—(IV) or linker-payloads (LP-I)-(LP-V) as described herein to a second compound, such as an macromolecule (i.e., COMP) described herein to form a conjugate of Formula (CONJ-I)—(CONJ-V) as described herein. In certain embodiments, the reactive group is designated RL herein. Reactive groups can react via any suitable reaction mechanism known to those of skill in the art. In certain embodiments, a reactive group (RG) reacts through a [3+2]alkyne-azide cycloaddition reaction, inverse-electron demand Diels-Alder ligation reaction, thiol-electrophile reaction, or carbonyl-oxyamine reaction, as described in detail herein. In certain embodiments, the reactive group (RG) comprises an alkyne, strained alkyne, tetrazine, thiol, para-acetyl-phenylalanine residue, oxyamine, maleimide, or azide. In certain embodiments, the reactive group is

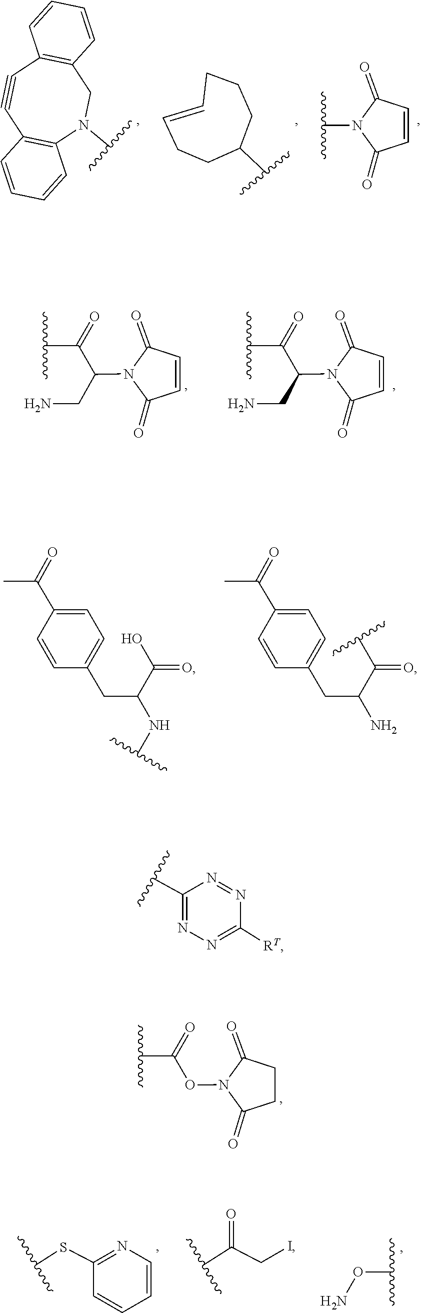

—N₃, or —SH; wherein R^{T} is lower alkyl. In certain embodiments, R^{T} is methyl, ethyl, or propyl. In some embodiments, R^{T} is methyl. In some embodiments, R^{T} is ethyl. In some embodiments, R^{T} is propyl. Additional reactive groups are described in, for example, U.S. Patent Application Publication No. 2014/0356385, U.S. Patent Application Publication No. 2013/0189287, U.S. Patent Application Publication No. 2013/0251783, U.S. Pat. Nos. 8,703,936, 9,145,361, 9,222,940, and 8,431,558.

After conjugation, a divalent residue of the reactive group (referred to as RL herein) is formed and is bonded to the residue of a second compound (e.g., COMP). The structure of the divalent residue is determined by the type of conjugation reaction employed to form the conjugate.

### [3+2] Alkyne-Azide Cycloaddition Reaction

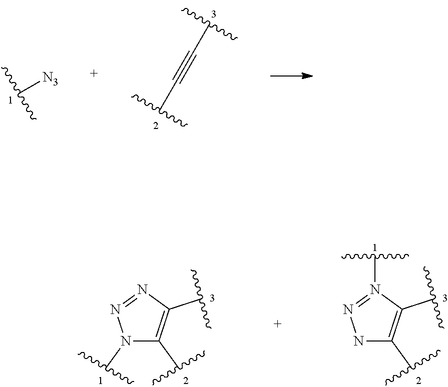

Advantageously, the compounds described herein comprising a conjugating alkyne group or an azide group facilitate selective and efficient reactions with a second compound comprising a complementary azide group or alkyne group. It is believed the azide and alkyne groups react in a 1,3-dipolar cycloaddition reaction to form a 1,2,3-triazolylene moiety which links the compounds described herein comprising an alkyne group or an azide group to the second compound. This reaction between an azide and alkyne to form a triazole is generally known to those in the art as a Huisgen cycloaddition reaction or a [3+2]alkyne-azide cycloaddition reaction.

The unique reactivity of azide and alkyne functional groups makes them useful for the selective modification of polypeptides and other biological molecules. Organic azides, particularly aliphatic azides, and alkynes are generally stable toward common reactive chemical conditions. In particular, both the azide and the alkyne functional groups are inert toward the side chains of the twenty common amino acids found in naturally-occurring polypeptides. It is believed that, when brought into close proximity, the “spring-loaded” nature of the azide and alkyne groups is revealed and azide and alkyne groups react selectively and efficiently via a [3+2]alkyne-azide cycloaddition reaction to generate the corresponding triazole. See, e.g., Chin J., et al., Science 301:964-7 (2003); Wang, Q., et al., J. Am. Chem. Soc. 125, 3192-3193 (2003); Chin, J. W., et al., J. Am. Chem. Soc. 124:9026-9027 (2002).

Because the [3+2]alkyne-azide cycloaddition reaction involves a selective cycloaddition reaction [see, e.g., Padwa, A., in COMPREHENSIVE ORGANIC SYNTHESIS, Vol. 4, (ed. Trost, B. M., 1991), pp. 1069-1109; Huisgen, R. in 1,3-DIPOLAR CYCLOADDITION CHEMISTRY, (ed. Padwa, A., 1984), pp. 1-176] rather than a nucleophilic substitution, the incorporation of non-naturally encoded amino acids bearing azide and alkyne-containing side chains permits the resultant polypeptides to be modified selectively at the position of the non-naturally encoded amino acid. Cycloaddition reactions involving azide or alkyne-containing compounds can be carried out at room temperature under aqueous conditions by the addition of Cu(II) (including, but not limited to, catalytic amounts of CuSO₄) in the presence of a reducing agent for reducing Cu(II) to Cu(I), in situ, in catalytic amounts. See, e.g., Wang, Q., et al., J. Am. Chem. Soc. 125, 3192-3193 (2003); Tornoe, C. W., et al., J. Org. Chem. 67:3057-3064 (2002); Rostovtsev, et al., Angew. Chem. Int. Ed. 41:2596-2599 (2002). Exemplary reducing agents include, but are not limited to, ascorbate, metallic copper, quinine, hydroquinone, vitamin K, glutathione, cysteine, Fe²⁺ Co²⁺, and an applied electric potential.

In certain embodiments when a conjugate is formed through a [3+2]alkyne-azide cycloaddition reaction, the divalent residue of the reactive group (e.g., RL) comprises a triazole ring or fused cyclic group comprising a triazole ring. In certain embodiments, when a conjugate is formed through a strain-promoted [3+2]alkyne-azide cycloaddition (SPAAC) reaction, the divalent residue of the reactive group (e.g., RL) is

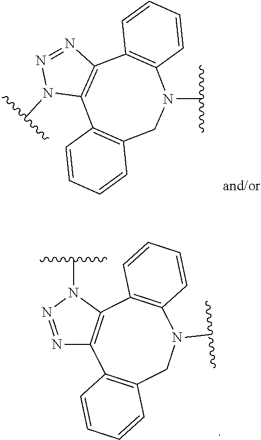 [SMILES: *N1Cc2ccccc2-c2c(nnn2*)-c2ccccc21 ; *N1Cc2ccccc2-c2nnn(*)c2-c2ccccc21]

If a conjugate of Formula (CONJ-I)—(CONJ-V) is formed by a [3+2]alkyne-azide cycloaddition, the conjugate encompasses both regioisomers. In certain embodiments, a conjugate of Formula (CONJ-I)—(CONJ-V) is a mixture of regioisomers formed from a [3+2]alkyne-azide cycloaddition.

### Inverse Electron Demand Ligation Reaction

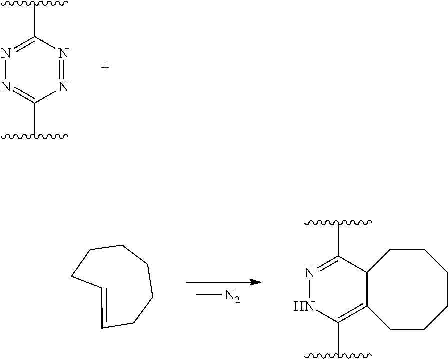

Advantageously, compounds comprising a terminal tetrazine or strained alkene group facilitate selective and efficient reactions with a second compound comprising a strained alkene or tetrazine group. It is believed that the tetrazine and strained alkene react in an inverse-demand Diels-Alder reaction followed by a retro-Diels-Alder reaction which links compounds comprising a terminal tetrazine or strained alkene group to the second compound. The reaction is believed to be specific, with little to no cross-reactivity with functional groups within biomolecules. The reaction may be carried out under mild conditions, for example, at room temperature and without a catalyst. This reaction between a tetrazine and a strained alkene is generally known to those in the art as a tetrazine ligation reaction.

In certain embodiments, when a conjugate is formed through a tetrazine inverse electron demand Diels-Alder ligation reaction, the divalent residue of the reactive group (e.g., RL) comprises a fused bicyclic ring having at least two adjacent nitrogen atoms in the ring. In certain embodiments, when a conjugate is formed through a tetrazine inverse electron demand Diels-Alder ligation reaction, the divalent residue of the reactive group (e.g., RL) is

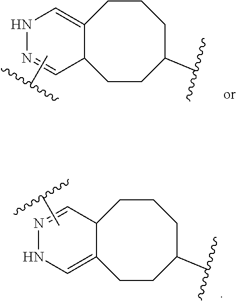

If a conjugate of Formula (CONJ-I)—(CONJ-V) is formed by an inverse electron demand ligation reaction, the conjugate encompasses both regioisomers. In certain embodiments, a conjugate of Formula (CONJ-I)—(CONJ-V) is a mixture of regioisomers formed from an inverse electron demand ligation reaction.

### Thiol Reactions

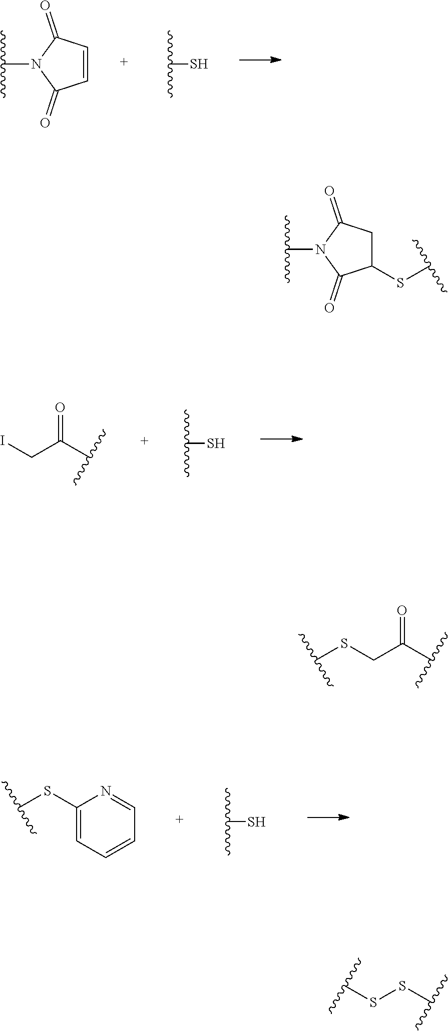

Advantageously, compounds comprising a terminal thiol group or suitable electrophilic or disulfide-forming group facilitate selective and efficient reactions with a second compound comprising a complementary electrophilic or disulfide-forming group or thiol group. These reactions are believed to be selective with little to no cross-reactivity with functional groups within biomolecules. In some embodiments, the thiol reaction does not include reaction of a maleimide group.

In certain embodiments, when a conjugate is formed through a thiol-maleimide reaction, the divalent residue of the reactive group comprises

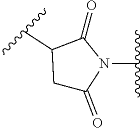 [SMILES: *C1CC(=O)N(*)C1=O]

and a sulfur linkage. In certain embodiments, when a conjugate is formed through a thiol-maleimide reaction,

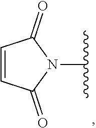 [SMILES: *N1C(=O)C=CC1=O]

the divalent residue of the reactive group (e.g., RL) is

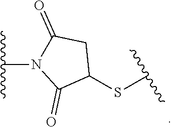 [SMILES: *SC1CC(=O)N(*)C1=O]

In certain embodiments, when a conjugate is formed through a thiol-maleimide reaction,

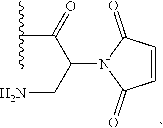 [SMILES: *C(=O)C(CN)N1C(=O)C=CC1=O]

the divalent residue of the reactive group (e.g., RL) is

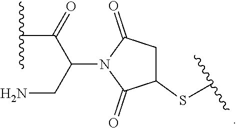 [SMILES: *SC1CC(=O)N(C(CN)C(*)=O)C1=O]

In certain embodiments, a conjugate is formed through a thiol-N-hydroxysuccinimide reaction using the following group:

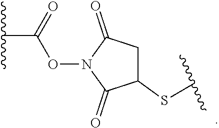 [SMILES: *SC1CC(=O)N(OC(*)=O)C1=O]

The reaction involved for formation of the conjugate comprises the following step.

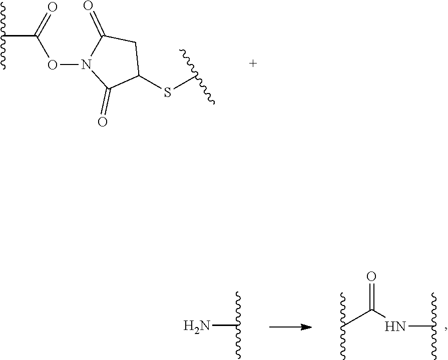

and the resulting divalent residue of the reactive group (e.g., RL) is

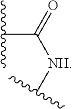 [SMILES: *NC(*)=O]

### Carbonyl-Oxyamine Reaction

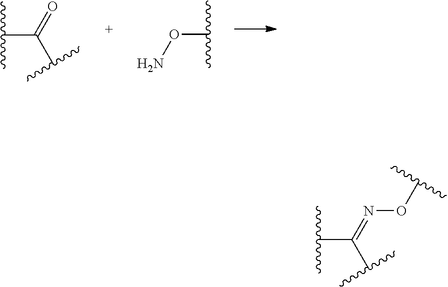

Advantageously, compounds comprising a terminal carbonyl or oxyamine group facilitate selective and efficient reactions with a second compound comprising an oxyamine or carbonyl group. It is believed that the carbonyl and oxyamine react to form an oxime linkage. The reaction is believed to be specific, with little to no cross-reactivity with functional groups within biomolecules.

In certain embodiments when a conjugate is formed through an oxime conjugation reaction, the divalent residue of the reactive group comprises a divalent residue of a non-natural amino acid. In certain embodiments when a conjugate is formed through an oxime conjugation reaction, the divalent residue of the reactive group (e.g., RL) is

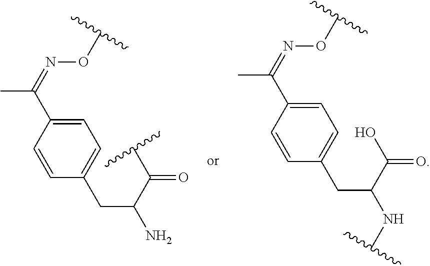 [SMILES: *O/N=C(/C)c1ccc(CC(N)C(*)=O)cc1 ; *NC(Cc1ccc(/C(C)=N\O*)cc1)C(=O)O]

In certain embodiments when a conjugate is formed through an oxime conjugation reaction, the divalent residue of the reactive group comprises an oxime linkage. In certain embodiments when a conjugate is formed through an oxime conjugation reaction, the divalent residue of the reactive group (e.g., RL) is

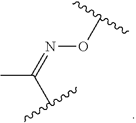 [SMILES: *O/N=C(\*)C]

### Other Reactions

Other suitable conjugation reactions are described in the literature. See, for example, Lang, K. and Chin, J. 2014, Bioorthogonal Reactions for Labeling Proteins, ACS Chem Biol 9, 16-20; Paterson, D. M. et al. 2014, Finding the Right (Bioorthogonal) Chemistry, ACS Chem Biol 9, 592-605; King, M. and Wagner, A. 2014, Developments in the Field of Bioorthogonal Bond Forming Reactions —Past and Present Trends, Bioconjugate Chem., 2014, 25 (5), pp 825-839; and Ramil, C. P. and Lin, Q., 2013, Bioorthogonal chemistry: strategies and recent developments, Chem Commun 49, 11007-11022.

### IV. Releasing Reactions

Releasing Reactions are reactions that act to release a biologically active portion of a compound or conjugate described herein from the compound or conjugate in vivo and/or in vitro. In certain embodiments, the released biologically active portion is a compound described elsewhere herein (e.g., cytotoxic agents), or a pharmaceutically acceptable salt, solvate, stereoisomer, or tautomer thereof. One example of a releasing reaction is an intramolecular reaction between an eliminator group and a release trigger group of a compound or conjugate described herein to release a biologically active portion of a compound or conjugate described herein. The eliminator group may itself devolve into two reactive components, as exemplified in these reactions where X is a drug having a heteroatom nitrogen or oxygen for linkage. Exemplary Releasing Reactions are depicted in the scheme below:

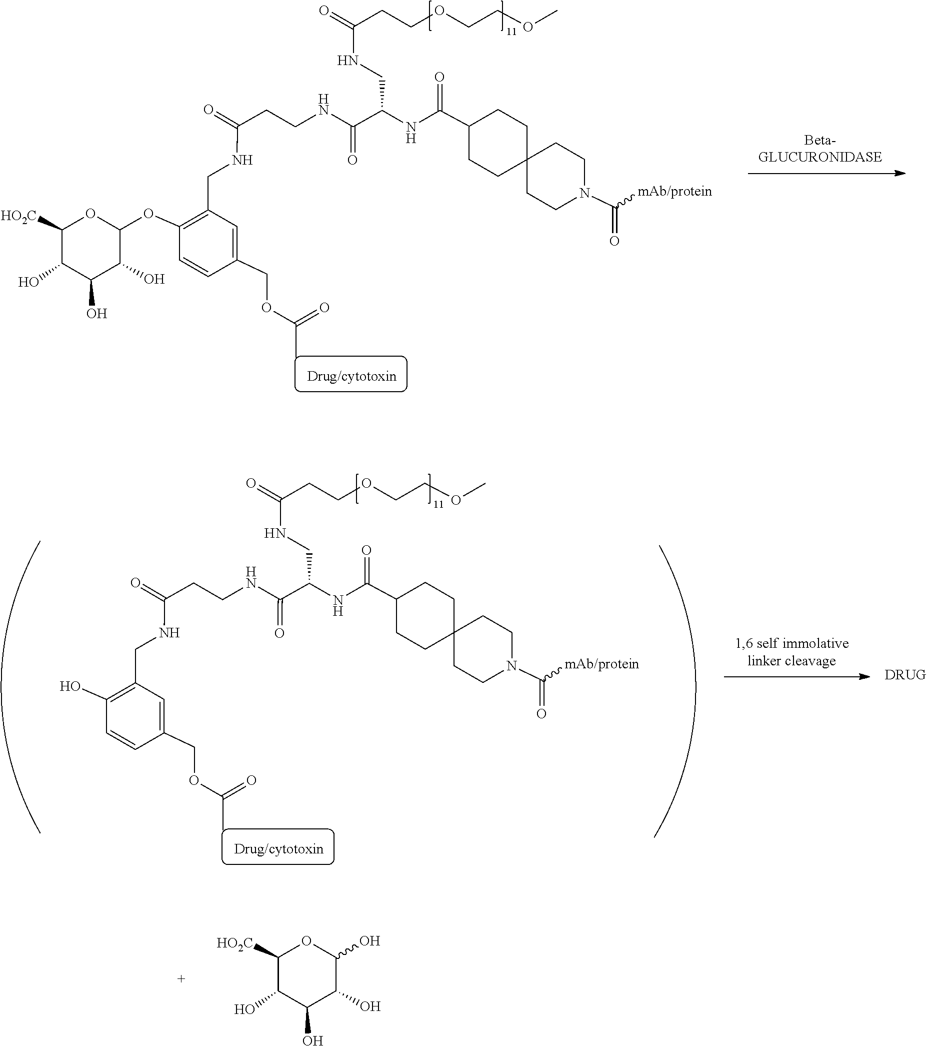

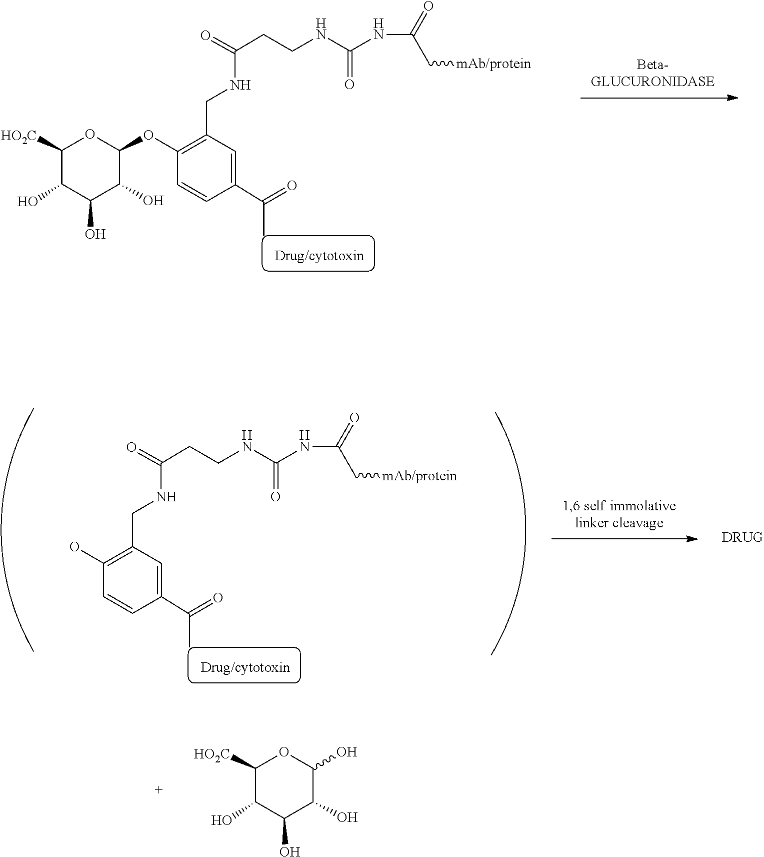

### V. Water Soluble Polymers

In certain embodiments, a compound or conjugate described herein comprises one or more water soluble polymers. A wide variety of macromolecular polymers and other molecules can be linked to the polypeptides described herein to modulate biological properties of the polypeptide, and/or provide new biological properties to the polypeptide. These macromolecular polymers can be linked to the polypeptide via a naturally encoded amino acid, via a non-naturally encoded amino acid, or any functional substituent of a natural or modified amino acid, or any substituent or functional group added to a natural or modified amino acid. The molecular weight of the polymer may include a wide range including, but not limited to, between about 100 Da and about 100,000 Da or more.

The polymer selected may be water soluble so that a protein to which it is attached does not precipitate in an aqueous environment, such as a physiological environment. The polymer may be branched or unbranched. In certain embodiments, for therapeutic use of the end-product preparation, the polymer will be pharmaceutically acceptable.

In certain embodiments, the proportion of polyethylene glycol molecules to polypeptide molecules will vary, as will their concentrations in the reaction mixture. In general, the optimum ratio (in terms of efficiency of reaction in that there is minimal excess unreacted protein or polymer) may be determined by the molecular weight of the polyethylene glycol selected and on the number of available reactive groups available. Regarding molecular weight, typically the higher the molecular weight of the polymer, the fewer number of polymer molecules which may be attached to the protein. Similarly, branching of the polymer should be taken into account when optimizing these parameters. Generally, the higher the molecular weight (or the more branches) the higher the polymer:protein ratio.

The water soluble polymer may be any structural form including, but not limited to, linear, forked, or branched. Typically, the water soluble polymer is a poly(alkylene glycol), such as poly(ethylene glycol) (PEG), but other water soluble polymers can also be employed. By way of example, PEG is used to describe certain embodiments.

PEG is a well-known, water soluble polymer that is commercially available or can be prepared by ring-opening polymerization of ethylene oxide according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of a PEG, and can be represented as linked to a polypeptide by the formula: X′O—(CH₂CH₂O)ₙ—CH₂CH₂—Y′ where n is an integer selected from 2 to 10,000, X′ is hydrogen or a terminal modification including, but not limited to, C₁₋₄ alkyl, and Y′ is the attachment point to the polypeptide.

In some cases, a PEG terminates on one end with hydroxy or methoxy, i.e., X′ is hydrogen or CH₃ (aka “methoxy PEG”). Alternatively, the PEG can terminate with a PEG reactive group, thereby forming a bifunctional polymer. Typical PEG reactive groups can include those reactive groups that are commonly used to react with the functional groups found in the twenty common amino acids (including, but not limited to, maleimide groups, activated carbonates (including, but not limited to, p-nitrophenyl ester), activated esters (including, but not limited to, N-hydroxysuccinimide, p-nitrophenyl ester, and aldehydes) as well as functional groups that are inert to the twenty common amino acids, but that react specifically with complementary functional groups present in non-naturally encoded amino acids (including, but not limited to, azide groups and/or alkyne groups). It is noted that the other end of the PEG, which is shown in the above formula by Y′, will attach either directly or indirectly to a polypeptide via a naturally occurring or non-naturally encoded amino acid. For instance, Y′ may be an amide, carbamate, or urea linkage to an amine group (including, but not limited to, the epsilon amine of lysine or the N-terminus) of the polypeptide. Alternatively, Y′ may be a maleimide linkage to a thiol group (including, but not limited to, the thiol group of cysteine). Alternatively, Y′ may be a linkage to a residue not commonly accessible via the twenty common amino acids. For example, an azide group on the PEG can be reacted with an alkyne group on the polypeptide to form a Huisgen [3+2]cycloaddition product. Alternatively, an alkyne group on the PEG can be reacted with an azide group present in a non-naturally encoded amino acid, such as the modified amino acids described herein, to form a similar product. In some embodiments, a strong nucleophile (including, but not limited to, hydrazine, hydrazide, hydroxylamine, or semicarbazide) can be reacted with an aldehyde or ketone group present in a non-naturally encoded amino acid to form a hydrazone, oxime, or semicarbazone, as applicable, which in some cases can be further reduced by treatment with an appropriate reducing agent. Alternatively, the strong nucleophile can be incorporated into the polypeptide via a non-naturally encoded amino acid and used to react preferentially with a ketone or aldehyde group present in the water soluble polymer.

Any molecular mass for a PEG can be used as practically desired including, but not limited to, from about 100 Daltons (Da) to 100,000 Da or more as desired (including, but not limited to, in certain embodiments 0.1-50 kDa or 10-40 kDa). Branched chain PEGs including, but not limited to, PEG molecules with each chain having a molecular weight (MW) ranging from 1-100 kDa (including, but not limited to, 1-50 kDa or 5-20 kDa) can also be used. A wide range of PEG molecules are described in the Shearwater Polymers, Inc. catalog, and the Nektar Therapeutics catalog, each incorporated herein by reference.

Generally, at least one terminus of the PEG molecule is available for reaction with the remainder of the compound of Formula (I), (IA), or (IB). For example, PEG derivatives bearing alkyne and azide moieties for reaction with amino acid side chains can be used to attach PEG to non-naturally encoded amino acids as described herein. If the non-naturally encoded amino acid comprises an azide, then the PEG will typically contain either an alkyne moiety to effect formation of the [3+2]cycloaddition product or an activated PEG species (i.e., ester, carbonate) containing a phosphine group to effect formation of the amide linkage. Alternatively, if the non-naturally encoded amino acid comprises an alkyne, then the PEG will typically contain an azide moiety to effect formation of the [3+2] Huisgen cycloaddition product. If the non-naturally encoded amino acid comprises a carbonyl group, the PEG will typically comprise a nucleophile (including, but not limited to, a hydrazide, hydrazine, hydroxylamine, or semicarbazide functionality) in order to effect formation of corresponding hydrazone, oxime, and semicarbazone linkages, respectively. In other alternatives, a reverse of the orientation of the reactive groups described herein can be used (i.e., an azide moiety in the non-naturally encoded amino acid can be reacted with a PEG derivative containing an alkyne).

In some embodiments, the polypeptide variant with a PEG derivative contains a chemical functionality that is reactive with the chemical functionality present on the side chain of the non-naturally encoded amino acid.

In certain embodiments, the water soluble polymer is an azide- or acetylene-containing polymer comprising a water soluble polymer backbone having an average molecular weight from about 800 Da to about 100,000 Da. The polymer backbone of the water-soluble polymer can be poly(ethylene glycol). However, it should be understood that a wide variety of water soluble polymers including, but not limited to, poly(ethylene)glycol and other related polymers, including poly(dextran) and poly(propylene glycol), are also suitable for use and that the use of the term “PEG” or “poly(ethylene glycol)” is intended to encompass and include all such molecules. The term “PEG” further includes, but is not limited to, poly(ethylene glycol) in any of its forms, including bifunctional PEG, multiarmed PEG, derivatized PEG, forked PEG, branched PEG, pendent PEG (i.e., PEG or related polymers having one or more functional groups pendent to the polymer backbone), or PEG with degradable linkages therein.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, glycerol oligomers, pentaerythritol, and sorbitol. The central branch moiety can also be derived from several amino acids, such as lysine. The branched poly(ethylene glycol) can be represented in general form as R—(-PEG-OH)ₘ in which R is derived from a core moiety, such as glycerol, glycerol oligomers, or pentaerythritol, and m represents the number of arms. Multi-armed PEG molecules, such as those described in U.S. Pat. Nos. 5,932,462; 5,643,575; 5,229,490; and 4,289,872; U.S. Pat. Appl. No. 2003/0143596; and WO 96/21469 and WO 93/21259, each of which is incorporated by reference herein in its entirety, can also be used as the polymer backbone.

Branched PEG can also be in the form of a forked PEG represented by PEG(-Y″CHZ₂)ₙ, where Y″ is a linking group and Z is an activated terminal group linked to CH by a chain of atoms of defined length. Yet another branched form, the pendant PEG, has PEG reactive groups, such as carboxyl, along the PEG backbone rather than at the end of PEG chains. In addition to these forms of PEG, the polymer can also be prepared with weak or degradable linkages in the backbone. For example, PEG can be prepared with ester linkages in the polymer backbone that are subject to hydrolysis. As shown herein, this hydrolysis results in cleavage of the polymer into fragments of lower molecular weight: -PEG-CO₂—PEG-+H₂O→PEG-CO₂H+HO-PEG-. It is understood by those skilled in the art that the term “poly(ethylene glycol)” or “PEG” represents or includes all the forms known in the art including, but not limited to, those disclosed herein. Many other polymers are also suitable for use. In some embodiments, polymer backbones that are water-soluble, with from two to about three hundred termini, are particularly suitable. Examples of suitable polymers include, but are not limited to, other poly(alkylene glycols), such as poly(propylene glycol) (“PPG”), copolymers thereof (including, but not limited to, copolymers of ethylene glycol and propylene glycol), terpolymers thereof, mixtures thereof, and the like. Although the molecular weight of each chain of the polymer backbone can vary, it is typically in the range of from about 800 Da to about 100,000 Da, often from about 6,000 Da to about 80,000 Da. Those of ordinary skill in the art will recognize that the foregoing list for substantially water-soluble backbones is by no means exhaustive and is merely exemplary, and that all polymeric materials having the qualities described herein are contemplated as being suitable for use. In some embodiments the polymer derivatives are “multi-functional,” meaning that the polymer backbone has at least two termini, and possibly as many as about 300 termini, functionalized or activated with a functional group. Multifunctional polymer derivatives include, but are not limited to, linear polymers having two termini, each terminus being bonded to a functional group which may be the same or different.

### VI. Uses of the Compounds, Conjugates, and Compositions

In one aspect, an effective amount of a compound or conjugate described herein or a composition thereof is used to treat a medical disorder or disease mediated by STING in a subject in need thereof. In one embodiment, the medical disorder or disease is a cellular proliferative disorder, including, but not limited to cancer. In another aspect, an effective amount of a compound or conjugate described herein or a composition thereof is used to induce an immune response in a subject need thereof. In yet another aspect, an effective amount of a compound or conjugate described herein or a composition thereof is used to induce STING-dependent type I interferon production in a subject in need thereof. In yet another aspect, an effective amount of a compound or conjugate described herein or a composition thereof is used to induce STING-dependent cytokine production in a subject in need thereof.

In one embodiment, an effective amount of a compound or conjugate described herein or a composition thereof is used to treat abnormal cellular proliferation, including, but not limited to, cancer. In certain embodiments, the term “cancer” includes, but is not limited to, the following cancers: epidermoid oral: buccal cavity, lip, tongue, mouth, pharynx, squamous cell carcinoma of the head and neck (HNSCC); cardiac: sarcoma (angiosarcoma, fibrosarcoma, rhabdomyosarcoma, liposarcoma), myxoma, rhabdomyoma, fibroma, lipoma, and teratoma; lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell lung cancer (NSCLC); gastrointestinal: gastric cancer, esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, vipoma), small bowel or small intestines (adenocarcinoma, lymphoma, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, microsatellite stable colorectal cancer (MSS CRC), rectum; genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, sarcoma, interstitial cell carcinoma, fibroma, fibroadenoma, adenomatoid tumors, lipoma), metastatic castrate-resistant prostate cancer (mCRPC), muscle-invasive urothelial cancer; Liver: hepatoma (hepatocellular carcinoma), cholangiocarcinoma, hepatoblastoma, hepatocellular adenoma, hemangioma, biliary passages; bone: osteogenic sarcoma (osteosarcoma), fibrosarcoma, malignant fibrous histiocytoma, Ewing's sarcoma, malignant lymphoma (reticulum cell sarcoma), malignant giant cell tumor osteochronfroma (osteocartilaginous exostoses), benign chondroma, chondroblastoma, chondromyxofibroma, osteoid osteoma and giant cell tumors; nervous system: skull (osteoma, hemangioma, granuloma, xanthoma, osteitis deformans), meninges (meningioma, meningiosarcoma, gliomatosis), brain (astrocytoma, medulloblastoma, glioma, germinoma (pinealoma), glioblastoma multiform, oligodendroglioma, schwannoma, retinoblastoma, congenital tumors), spinal cord neurofibroma, meningioma, glioma, sarcoma); gynecological: uterus (endometrial carcinoma), cervix (cervical cancer, cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast, triple-negative breast cancer (TNBC), platinum-resistant epithelial ovarian cancer (EOC); hematologic: blood (myeloid leukemia (acute and chronic), acute lymphoblastic leukemia, chronic lymphocytic leukemia, myeloproliferative diseases, multiple myeloma (MM), myelodysplastic syndrome), Hodgkin's disease, non-Hodgkin's lymphoma (malignant lymphoma) hairy cell; lymphoid disorders (e.g., mantle cell lymphoma, Waldenström's macroglobulinemia, Marginal zone lymphoma, and Follicular lymphoma); skin: malilymphgnant melanoma, basal cell carcinoma, squamous cell carcinoma, Karposi's sarcoma, keratoacanthoma, moles dysplastic nevi, lipoma, angioma, dermatofibroma, keloids, psoriasis; thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; Adrenal glands: neuroblastoma; and metatstaic melanoma.

In certain embodiments, the cancer is selected from acute myeloid leukemia, breast cancer, colorectal cancer, glioma, head and neck squamous cell carcinoma, lung cancer, including non-small cell lung cancer, head and neck cancer, lymphoma, including a malignant lymphoma, melanoma, nasopharyngeal carcinoma, ovary cancer, pancreatic cancer, prostate cancer, urothelial cancer, and tongue squamous cell carcinoma.

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas. Additional examples of solid tumors include, but are not limited to, squamous cell carcinoma, colon cancer, breast cancer, prostate cancer, lung cancer, liver cancer, pancreatic cancer, and melanoma. In one embodiment, the solid tumor is an advanced solid tumor.

Non-limiting examples of cancers that can be treated using the compounds described herein include, but are not limited to, acoustic neuroma, an adenocarcinoma, adrenal gland cancer, anal cancer, an angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma, glioma, astrocytoma, oligodendroglioma; medulloblastoma), bronchus cancer, carcinoid tumor, cervical cancer, choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), eye cancer (e.g., intraocular melanoma, retinoblastoma), familiar hypereosinophilia, gallbladder cancer, gastric cancer (e.g., stomach adenocarcinoma), gastrointestinal stromal tumor (GIST), head and neck cancer (e.g., head and neck squamous cell carcinoma), oral cancer (e.g., oral squamous cell carcinoma (OSCC)), throat cancer (e.g., laryngeal cancer, pharyngeal cancer, nasopharyngeal cancer, oropharyngeal cancer), a hematopoietic cancer, heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (C-L), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), Wilms' tumor, urethral cancer, vaginal cancer and vulvar cancer (e.g., Paget's disease of the vulva).

In one embodiment, the cancer is a sarcoma, including, but not limited to, Ewing's sarcoma, Kaposi's sarcoma, liposarcoma, myosarcomas, peripheral neuroepithelioma, synovial sarcoma, gliomas, astrocytomas (including anaplastic astrocytoma, diffuse astrocytoma and low-grade astrocytoma), oligodendrogliomas, gliobastomas, neuroblastomas, ganglioneuromas, gangliogliomas, medulloblastomas, pineal cell tumors, meningiomas, meningeal sarcomas, neurofibromas, and Schwannomas.

In one embodiment, the cancer is a hematopoietic cancer, including, but not limited to, leukemia, such as acute lymphocytic leukemia (ALL), also known as acute lymphoblastic leukemia or acute lymphoid leukemia (e.g., B-cell ALL, T-cell ALL), acute myelocytic leukemia (AML) (e.g., B-cell AML, T-cell AML), acute granulocytic leukemia, chronic myelocytic leukemia (CML) (e.g., B-cell CML, T-cell CML), a chronic lymphocytic leukemia (CLL) (e.g., B-cell CLL, T-cell CLL), and hairy cell leukemia (HCL). In one embodiment, the hematopoietic cancer is a lymphoma, such as Hodgkin lymphoma (HL) (e.g., B-cell HL, T-cell HL), non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma), and a mixture of one or more leukemia/lymphoma as described above. Additional leukemias and lymphomas include T-cell lineage acute lymphoblastic leukemia (T-ALL), T-cell lineage lymphoblastic lymphoma (T—LL), peripheral T-cell lymphoma, Adult T-cell leukemia, Pre-B ALL, Pre-B lymphomas, large B-cell lymphoma, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, splenic diffuse red pulp small B-cell lymphoma; lymphoplasmacytic lymphoma;

In certain embodiments, the cancer that is treated using the disclosed compounds is selected from adenosarcoma, adrenal cancer, adrenocortical carcinoma, bile duct cancer, bone cancer, bone marrow cancer, brain stem glioma, breast cancer, (including, but not limited to triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, or inflammatory breast cancer (IBC), mesothelioma metastatic breast cancer), colorectal cancer, cutaneous lymphoma, cutaneous melanoma, ductal carcinoma in situ (DCIS), endometrial cancer, epithelioid sarcoma, esophageal cancer, extrahepatic eye cancer, fallopian tube cancer, fibrosarcoma, gastric cancer, gastrointestinal cancer, gastrointestinal carcinoid cancer, gastrointestinal stromal tumors (GIST), germ cell tumor glioblastoma multiforme (GBM), glioma, hairy cell leukemia, hemangioendothelioma, hypopharyngeal cancer, infiltrating ductal carcinoma (IDC), infiltrating lobular carcinoma (ILC), intestinal cancer, intrahepatic bile duct cancer, invasive/infiltrating breast cancer, Islet cell cancer, jaw cancer, kidney cancer, laryngeal cancer, leiomyosarcoma, leptomeningeal metastases, lip cancer, liposarcoma, liver cancer, lobular carcinoma in situ, lung cancer, lymph node cancer, lymphoma, male breast cancer, medullary carcinoma, medulloblastoma, melanoma, meningioma, Merkel cell carcinoma, mesenchymal chondrosarcoma, mesenchymous, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, plasma cell myeloma, solitary plasmacytoma of bone, and extraosseous plasmacytoma.

In other embodiments, the cell-proliferation disorder is selected from benign papillomatosis, benign neoplastic diseases and gestational trophoblastic diseases. In certain embodiments, the benign neoplastic disease is selected from skin papilloma (warts) and genital papilloma. In certain embodiments, the gestational trophoblastic disease is selected from the group consisting of hydatidiform moles, and gestational trophoblastic neoplasia (e.g., invasive moles, choriocarcinomas, placental-site trophoblastic tumors, and epithelioid trophoblastic tumors).

In an alternative aspect, an effective amount of a compound or conjugate described herein or a composition thereof is used to treat a medical disorder or disease mediated by STING in a subject in need thereof wherein the disorder or disease is a viral infection, for example, a double stranded DNA virus. In certain embodiments, the virus is from the family Herpesviridae, including but not limited to herpes simplex virus-1 (HSV-1), herpes simplex virus-2 (HSV-2), varicella zoster virus (VZV), epstein-Barr virus (EBV), cytomegalovirus (CMV), and Kaposi's sarcoma-associated herpesvirus (KSHV). In one embodiment, the virus is an adenovirus. In certain embodiments, the virus is from the family Papillomaviridae, including but not limited to human papillomavirus (HPV). In an alternative aspect, the viral infection is an RNA viral infection, for example, a virus from the Flaviviridae family, including Flaviviruses (for example, Yellow fever virus, West Nile virus, Dengue virus, and Zika virus) and Hepacivirus (for example, hepatitis B and hepatitis C).

The compound or conjugate described herein or a composition thereof can be administered at any dose deemed suitable by the practitioner of skill. In certain embodiments, the dose is 0.1-1000 mg/kg. In certain embodiments, the dose is 0.1-900 mg/kg. In certain embodiments, the dose is 0.1-800 mg/kg. In certain embodiments, the dose is 0.1-700 mg/kg. In certain embodiments, the dose is 0.1-600 mg/kg. In certain embodiments, the dose is 0.1-500 mg/kg. In certain embodiments, the dose is 0.1-400 mg/kg. In certain embodiments, the dose is 0.1-300 mg/kg. In certain embodiments, the dose is 0.1-200 mg/kg. In certain embodiments, the dose is 0.1-100 mg/kg. In certain embodiments, the dose is selected from the group consisting of 100 mg/kg, 200 mg/kg, 300 mg/kg, 450 mg/kg, 600 mg/kg, 800 mg/kg, and 1000 mg/kg. In certain embodiments, the dose is about 25 mg/kg. In certain embodiments, the dose is about 50 mg/kg. In certain embodiments, the dose is about 75 mg/kg. In certain embodiments, the dose is about 100 mg/kg. In certain embodiments, the dose is about 150 mg/kg. In certain embodiments, the dose is about 200 mg/kg. In certain embodiments, the dose is about 250 mg/kg. In certain embodiments, the dose is about 300 mg/kg. In certain embodiments, the dose is about 400 mg/kg. In certain embodiments, the dose is about 450 mg/kg. In certain embodiments, the dose is about 500 mg/kg. In certain embodiments, the dose is about 600 mg/kg. In certain embodiments, the dose is about 700 mg/kg. In certain embodiments, the dose is about 750 mg/kg. In certain embodiments, the dose is about 800 mg/kg. In certain embodiments, the dose is about 900 mg/kg. In certain embodiments, the dose is about 1000 mg/kg.

The dose can be administered on a schedule deemed suitable by the person of skill in the art. In certain embodiments, the dose is administered once per day. In certain embodiments, the dose is administered twice per day. In certain embodiments, the dose is administered three times per day. In certain embodiments, the dose is administered four times per day. In certain embodiments, the dose is administered in divided doses. In certain embodiments, the dose is administered in two divided doses per day. In certain embodiments, the dose is administered in three divided doses per day. In certain embodiments, the dose is administered in four divided doses per day.

Dosing can continue for any length of time deemed suitable by the person of skill in the art. In certain embodiments, the dose is administered daily for fourteen days. In certain embodiments, the dose is administered daily for thirteen days. In certain embodiments, the dose is administered daily for twelve days. In certain embodiments, the dose is administered daily for eleven days. In certain embodiments, the dose is administered daily for ten days. In certain embodiments, the dose is administered daily for nine days. In certain embodiments, the dose is administered daily for eight days. In certain embodiments, the dose is administered daily for seven days. In certain embodiments, the dose is administered daily for six days. In certain embodiments, the dose is administered daily for five days. In certain embodiments, the dose is administered daily for four days. In certain embodiments, the dose is administered daily for three days. In certain embodiments, the dose is administered daily for two days. In certain embodiments, the dose is administered for one day.

In the dosing schedule, the doses can be administered on consecutive days or cyclically, according to the judgment of the practitioner of skill. In certain embodiments, the doses are administered on consecutive days. In certain embodiments, the doses are administered with an interval between doses. In certain embodiments, the interval is one day. In certain embodiments, the interval is two days. In certain embodiments, the interval is three days. In certain embodiments, the interval is four days. In certain embodiments, the interval is five days. In certain embodiments, the interval is six days.

In certain embodiments, the dose is administered weekly. In certain embodiments, the dose is administered twice per week. In certain embodiments, the dose is administered three times per week.

In certain embodiments, the dose(s) are administered for a period of time with a first interval between dose(s), and then the dose(s) are re-administered for a period of time following the first interval between dose(s), wherein this dosing regimen can be repeated (i.e., cyclicly or cyclically, for example, after a second, third, etc. interval between subsequent administrations of dose(s)) according to the judgment of the practitioner of skill. For example, in one embodiment, a first dose is administered for one week, followed by a first interval of one week without the first dose administration; then, a second dose is re-administered for another week, followed by a second interval of one week without the first or second dose administration, and so on cyclically. Other perturbations for first, second, third, etc. dose(s) followed by perturbations for first, second, third, etc. interval(s), and combinations thereof, are contemplated herein as would be appreciated by the practitioner of skill and the need of the patient. For example, in one embodiment, a first dose is administered daily for one week, followed by a first interval of three weeks without the first daily dose administration; then, a second dose is re-administered biweekly for another week, followed by a second interval of four weeks without the first daily or second biweekly dose administration, and so on cyclically.

The effective amount of a compound or conjugate described herein or a composition thereof can be administered by any route of administration deemed suitable by the practitioner of skill. In certain embodiments, the dose is administered orally. Formulations and techniques for administration are described in detail below.

### VI. Pharmaceutical Compositions

The compounds or conjugates described herein can be formulated into pharmaceutical compositions that further comprise a pharmaceutically acceptable carrier, diluent, excipient, or vehicle. In one embodiment, this disclosure provides a pharmaceutical composition comprising a compound or conjugates described herein, and a pharmaceutically acceptable carrier, diluent, excipient, or vehicle. In one embodiment, provided herein are pharmaceutical compositions comprising an effective amount of a compound or conjugates described herein and one or more pharmaceutically acceptable carriers, diluents, excipients, or vehicles.

According to another embodiment, the description provides a composition comprising a compound or conjugates described herein and a pharmaceutically acceptable carrier, or vehicle. Pharmaceutical compositions of this description comprise a therapeutically effective amount of a compound of Formula (I)—(IV) or a conjugate of Formula (CONJ-I)—(CONJ-V) or a pharmaceutically acceptable salt, stereoisomer, or salt thereof.

It also will be appreciated that certain compounds and conjugates of this disclosure can exist in free form for treatment, or where appropriate, as a pharmaceutically acceptable derivative (e.g., a salt) thereof. According to this disclosure, a pharmaceutically acceptable derivative includes, but is not limited to, pharmaceutically acceptable prodrugs, salts, esters, salts of such esters, or any other adduct/educt or derivative that upon administration to a patient in need is capable of providing, directly or indirectly, a compound as otherwise described herein, or a metabolite or residue thereof.

As used herein, the term “pharmaceutically acceptable salt” or “salt” refers to those salts that are, within the scope of sound medical judgement, suitable for use in contact with the tissues of humans and lower animals without undue toxicity, irritation, allergic response, and the like.

Pharmaceutically acceptable salts are well known in the art. For example, S. M. Berge et al., describe pharmaceutically acceptable salts in detail in J. Pharmaceutical Sciences 1977, 66, 1-19, incorporated herein by reference. Pharmaceutically acceptable salts of the compounds of this description include those derived from suitable inorganic and organic acids and bases. Examples of pharmaceutically acceptable, nontoxic acid addition salts include salts of an amino group formed with inorganic acids such as hydrochloric acid, hydrobromic acid, phosphoric acid, sulfuric acid, and perchloric acid; or with organic acids such as acetic acid, oxalic acid, maleic acid, tartaric acid, citric acid, succinic acid, or malonic acid; or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4 salts. This description also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A pharmaceutically acceptable carrier may contain inert ingredients that do not unduly inhibit the biological activity of the compounds. The pharmaceutically acceptable carriers should be biocompatible, for example, non-toxic, non-inflammatory, non-immunogenic, or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, the use of such conventional carrier medium is contemplated to be within the scope of this description. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain, and headache), hair loss, asthenia, dizziness, extra-pyramidal symptoms, akathisia, cardiovascular disturbances, and sexual dysfunction.

Some examples of materials that can serve as pharmaceutically acceptable carriers include, but are not limited to, ion exchangers, alumina, aluminum stearate, lecithin, serum proteins (such as human serum albumin), buffer substances (such as tween 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring, and perfuming agents. Preservatives and antioxidants can also be present in the composition, according to the judgment of the formulator.

The compositions of the present invention may be administered orally, parenterally, by inhalation spray, topically, rectally, nasally, buccally, vaginally or via an implanted reservoir. As used herein, the term “parenteral” includes subcutaneous, intravenous, intramuscular, intra-articular, intra-synovial, intrasternal, intrathecal, intraocular, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation also may be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions also may contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers that are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents also may be added.

Alternatively, the pharmaceutically acceptable compositions of this invention may be administered in the form of suppositories for rectal or vaginal administration. These can be prepared by mixing the agent with a suitable non-irritating excipient that is solid at room temperature but liquid at rectal temperature and therefore will melt in the rectum or vaginal cavity to release the drug. Such materials include cocoa butter, polyethylene glycol or a suppository wax that is solid at ambient temperature but liquid at body temperature and therefore melt in the rectum or vaginal cavity and release the active compound.

The pharmaceutically acceptable compositions of this invention also may be administered topically, especially when the target of treatment includes areas or organs readily accessible by topical application, including diseases of the eye, skin, or lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches also may be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutically acceptable compositions may be formulated, e.g., as micronized suspensions in isotonic, pH adjusted sterile saline or other aqueous solution, or, preferably, as solutions in isotonic, pH adjusted sterile saline or other aqueous solution, either with or without a preservative such as benzylalkonium chloride. Alternatively, for ophthalmic uses, the pharmaceutically acceptable compositions may be formulated in an ointment such as petrolatum. The pharmaceutically acceptable compositions of this invention also may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, the compositions of this disclosure are administered orally. The pharmaceutically acceptable compositions of this description may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents also may be added.

Liquid dosage forms for oral administration include, but are not limited to, pharmaceutically acceptable emulsions, microemulsions, solutions, suspensions, syrups, and elixirs. In addition to the active compounds herein, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions also can include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound herein is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets, and pills, the dosage form also may comprise buffering agents.

Solid compositions of a similar type also may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms also can be of a composition such that they release the active ingredient(s) only, for example, in a certain part of the intestinal tract, optionally, in a delayed manner. Examples of embedding compositions that can be used include polymeric substances and waxes. Solid compositions of a similar type also may be employed as fillers in soft and hard-filled gelatin capsules using such excipients as lactose or milk sugar as well as high molecular weight polyethylene glycols and the like.

The compounds and conjugates described herein also can be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose, or starch. Such dosage forms also may comprise, as is normal practice, additional substances other than inert diluents, for example, tableting lubricants and other tableting aids such a magnesium stearate and microcrystalline cellulose. In the case of capsules, tablets and pills, the dosage forms also may comprise buffering agents. They may optionally contain opacifying agents and also can be of a composition such that they release the active ingredient(s) only, for example, in a certain part of the intestinal tract, optionally, in a delayed manner. Examples of embedding compositions that can be used include polymeric substances and waxes.

The compounds and conjugates of the description are formulated in dosage unit form for ease of administration and uniformity of dosage. As used herein, the phrase “dosage unit form” refers to a physically discrete unit of agent appropriate for the patient to be treated. It will be understood, however, that the total daily usage of the compounds and compositions of this disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific compound employed; the duration of the treatment; drugs used in combination or coincidental with the specific compound employed, and like factors well known in the medical arts.

The amount of the compounds or conjugates of this disclosure that may be combined with the carrier materials to produce a composition in a single dosage form will vary depending upon the host treated, the particular mode of administration, and other factors. The compositions should be formulated so that a dosage of between 0.01-100 mg/kg body weight/day of the compound or inhibitor can be administered to a patient receiving these compositions.

### VII. Combinations

The compounds or conjugates described herein or compositions thereof can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually. Additional therapeutically active agents include, but are not limited to, small organic molecules such as drug compounds (e.g., compounds approved by the Food and Drugs Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells. In certain embodiments, the additional therapeutically agent is a cancer agent (e.g., a biotherapeutic or chemo therapeutic cancer agent).

In one embodiment, the additional active agent(s) may be one or more agents selected from the group consisting of STING agonist compounds, anti-viral compounds, antigens, adjuvants, anti-cancer agents, CTLA-4, LAG-3 and PD-1 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including, but not limited to anti-cancer vaccines.

In one embodiment, the additional therapeutically active agent is an anti-inflammatory agent. In one aspect of this embodiment, the additional therapeutically active agent is an immune modulator, including but not limited to a checkpoint inhibitor, for example, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor. In certain aspects, the immune modulator is an antibody, such as a monoclonal antibody.

PD-1 inhibitors that blocks the interaction of PD-1 and PD-L1 by binding to the PD-1 receptor, and in turn inhibit immune suppression include, for example, nivolumab (Opdivo), pembrolizumab (Keytruda), pidilizumab, AMP-224 (AstraZeneca and Medlmmune), PF-06801591 (Pfizer), MEDIO680 (AstraZeneca), PDR001 (Novartis), REGN2810 (Regeneron), SHR-12-1 (Jiangsu Hengrui Medicine Company and Incyte Corporation), TSR-042 (Tesaro), and the PD-L1/VISTA inhibitor CA-170 (Curis Inc.). PD-L1 inhibitors that block the interaction of PD-1 and PD-L1 by binding to the PD-L1 receptor, and in turn inhibits immune suppression, include for example, atezolizumab (Tecentriq), durvalumab (AstraZeneca and Medlmmune), KN035 (Alphamab), and BMS-936559 (Bristol-Myers Squibb). CTLA-4 checkpoint inhibitors that bind to CTLA-4 and inhibits immune suppression include, but are not limited to, ipilimumab, tremelimumab (AstraZeneca and Medlmmune), AGEN1884 and AGEN2041 (Agenus). LAG-3 checkpoint inhibitors, include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). An example of a TIM-3 inhibitor is TSR-022 (Tesaro).

In an alternative embodiment, the PD-1 antagonist as a second therapeutic agent is a monoclonal antibody (mAb), or antigen binding fragment thereof, which specifically binds to PD-1 or PD-L1, and preferably specifically binds to human PD-1 or human PD-L1. The mAb may be a human antibody, a humanized antibody, or a chimeric antibody and may include a human constant region. In some embodiments, the human constant region is selected from the group consisting of IgG1, IgG2, IgG3, and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv, and Fv fragments.

In one embodiment, the additional therapeutically active agent is a cytotoxic agent or a chemotherapy agent. Examples of cytotoxic agents that may be used in combination with the compounds or pharmaceutically acceptable salts described herein, include, but are not limited to, arsenic trioxide (sold under the tradename TRISENOX®), asparaginase (also known as L-asparaginase, and Erwinia L-asparaginase, sold under the tradenames ELSPAR® and KIDROLASE®).

Chemotherapeutic agents that may be used in combination with the compounds or pharmaceutically acceptable salts described herein include abiraterone acetate, altretamine, anhydrovinblastine, auristatin, bexarotene, bicalutamide, BMS 184476, 2,3,4,5,6-pentafluoro-N-(3-fluoro-4-methoxyphenyl) benzene sulfonamide, bleomycin, N,N-dimethyl-L-valyl-L-valyl-N-methyl-L-valyl-L-prolyl-1-Lproline-t-butylamide, cachectin, cemadotin, chlorambucil, cyclophosphamide, 3′,4′-didehydro-4′deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyurea andtaxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, nivolumab, onapristone, paclitaxel, pembrolizumab, prednimustine, procarbazine, RPR109881, stramustine phosphate, tamoxifen, tasonermin, taxol, tretinoin, vinblastine, vincristine, vindesine sulfate, and vinflunine. Such chemotherapeutic agents may be provided as a pharmaceutically acceptable salt, where appropriate. In one embodiment, the additional therapeutically active agent is a vascular endothelial growth factor (VEGF) receptor inhibitors including, but are not limited to, bevacizumab (AVASTIN), axitinib, brivanib alaninate ((S)-((R)-1-(4-(4-fluoro-2-methyl-1H-indol-5-yloxy)-5-methylpyrrolo[2,1-f][1,2,4]triazin-6-yloxy) propan-2-yl)2-aminopropanoate, also known as BMS-582664), motesanib (N-(2,3-dihydro-3,3-dimethyl-1H-indol-6-yl)-2-[(4-pyridinylmethyl)amino]-3-pyridinecarboxamide), pasireotide, and sunitinib (SUTENT), sorafenib (NEXAVAR).

In one embodiment, the additional therapeutically active agent is a topoisomerase II inhibitor, including, but are not limited to, etoposide (also known as VP-16 and etoposide phosphate, sold under the tradenames TOPOSAR, VEPESID, and ETOPOPHOS), and teniposide (also known as VM-26, sold under the tradename VUMON).

In one embodiment, the additional therapeutically active agent is an alkylating agent, including, but are not limited to, 5-azacytidine (VIDAZA), decitabine (DECOGEN), temozolomide (TEMCAD, TEMODAR, and TEMODAL), dactinomycin (also known as actinomycin-D and sold under the tradename COSMEGEN), melphalan (also known as L-PAM, L-sarcolysin, and phenylalanine mustard, sold under the tradename ALKERAN), altretamine (also known as hexamethylmelamine (HMM), sold under the tradename HEXALEN), carmustine (BCNU), bendamustine (TREANDA), busulfan (BUSULFEX® and MYLERAN®), carboplatin (PARAPLATIN®), lomustine (also known as CCNU, sold under the tradename CEENU®), cisplatin (also known as CDDP, sold under the tradenames PLATINOL® and PLATINOL®-AQ), chlorambucil (LEUKERAN®), cyclophosphamide (CYTOXAN® and NEOSAR®), dacarbazine (also known as DTIC, DIC and imidazole carboxamide, sold under the tradename DTIC-DOME®), altretamine (also known as hexamethylmelamine (HMM) sold under the tradename HEXALEN®), ifosfamide (IFEX®), procarbazine (MATULANE®), mechlorethamine (also known as nitrogen mustard, mustine and mechloroethamine hydrochloride, sold under the tradename MUSTARGEN®), streptozocin (ZANOSAR®), thiotepa (also known as thiophosphoamide, TESPA and TSPA, and sold under the tradename THIOPLEX®. Such alkylating agents may be provided as a pharmaceutically acceptable salt, where appropriate.

Examples of anti-tumor antibiotics include, but are not limited to, doxorubicin (sold under the tradenames ADRIAMYCIN® and RUBEX®), bleomycin (sold under the tradename LENOXANE®), daunorubicin (also known as dauorubicin hydrochloride, daunomycin, and rubidomycin hydrochloride, sold under the tradename CERUBIDINE®), daunorubicin liposomal (daunorubicin citrate liposome, sold under the tradename DAUNOXOME®), mitoxantrone (also known as DHAD, sold under the tradename NOVANTRONE®), epirubicin (sold under the tradename ELLENCE™), idarubicin (sold under the tradenames IDAMYCIN®, IDAMYCIN PFS®), and mitomycin C (sold under the tradename MUTAMYCIN®). Such anti-tumor antibiotics may be provided as a pharmaceutically acceptable salt, where appropriate.

In one embodiment, the additional therapeutically active agent is an anti-metabolite including, but are not limited to, claribine (2-chlorodeoxyadenosine, LEUSTATIN®), 5-fluorouracil (ADRUCTL®), 6-thioguanine (PURINETHOL®), pemetrexed (ALIN4TA®), cytarabine (also known as arabinosylcytosine (Ara-C), sold under the tradename CYTOSAR-U®), cytarabine liposomal (also known as Liposomal Ara-C, sold under the tradename DEPOCYT™), decitabine (DACOGEN®), hydroxyurea and (HYDREA®, DROXIA™ and MYLOCEL™), fludarabine (FLUDARA®), floxuridine FUDR®), cladribine (also known as 2-chlorodeoxyadenosine (2-CdA) sold under the tradename LEUSTATIN™), methotrexate (also known as amethopterin, methotrexate sodium (MTX), sold under the tradenames RHEUMATREX® and TREXALL™), and pentostatin (NIPENT®). Such anti-metabolites may be provided as a pharmaceutically acceptable salt, where appropriate.

In one embodiment, the additional therapeutically active agent is a retinoid including, but are not limited to, alitretinoin (PANRETIN®), tretinoin (all-trans retinoic acid, also known as ATRA, sold under the tradename VESANOID®), isotretinoin (13-c/s-retinoic acid, sold under the tradenames ACCUTANE®, AMNESTEEM®, CLARAVIS®, CLARUS®, DECUTAN®, ISOTANE®, IZOTECH®, ORATANE®, ISOTRET®, and SOTRET®), and bexarotene (TARGRETIN®).

The amount of additional therapeutic agent present in the compositions of this disclosure will be no more than the amount that would normally be administered in a composition comprising that therapeutic agent as the only active agent. The amount of additional therapeutic agent in the presently disclosed compositions will range from about 50% to 100% of the amount normally present in a composition comprising that agent as the only therapeutically active agent.

In certain embodiments, a compound or conjugate described herein or a composition thereof is administered in conjunction with one or more additional therapeutic agents including vaccines intended to stimulate an immune response. Antigens and adjuvants that may be used in combination with the compounds or pharmaceutically acceptable salts described herein include B7 costimulatory molecule, interleukin-2, interferon-y, GM-CSF, CTLA-4 antagonists, OX-40/OX-40 ligand, CD40/CD40 ligand, sargramostim, levamisol, vaccinia virus, Bacille Calmette-Guerin (BCG), liposomes, alum, Freund's complete or incomplete adjuvant, detoxified endotoxins, mineral oils, surface active substances such as lipolecithin, pluronic polyols, polyanions, peptides, and oil or hydrocarbon emulsions. Adjuvants, such as aluminum hydroxide or aluminum phosphate, can be added to increase the ability of the vaccine to trigger, enhance, or prolong an immune response. Additional materials, such as cytokines, chemokines, and bacterial nucleic acid sequences, like CpG, a toll-like receptor (TLR) 9 agonist as well as additional agonists for TLR 2, TLR 4, TLR 5, TLR 7, TLR 8, TLR9, including lipoprotein, LPS, monophosphoryllipid A, lipoteichoic acid, imiquimod, resiquimod, and in addition retinoic acid-inducible gene I (RIG-I) agonists such as poly I:C, used separately or in combination with the described compositions are also potential adjuvants. Such antigens and adjuvants as second therapeutically active agents may be provided as a pharmaceutically acceptable salt, where appropriate.

Anti-viral compounds that may be used in combination with the compounds or pharmaceutically acceptable salts described herein include hepatitis B virus (HBV) inhibitors, hepatitis C virus (HCV) protease inhibitors, HCV polymerase inhibitors, HCV NS4A inhibitors, HCV NS5A inhibitors, HCV NS5b inhibitors, and human immunodeficiency virus (HIV) inhibitors. Such anti-viral compounds may be provided as a pharmaceutically acceptable salt, where appropriate.

### VIII. Articles of Manufacture and Kits

Also provided are articles of manufacture comprising any of the compounds or conjugates described herein or compositions thereof. The articles of manufacture include suitable containers or packaging materials for the compounds or pharmaceutical compositions. Examples of a suitable container include, but are not limited to, a bottle, a vial, a syringe, an intravenous bag, or a tube.

Also provided are kits comprising any of the compounds or conjugates described herein or compositions thereof. The kits can contain the compounds or pharmaceutical compositions in suitable containers or packaging materials, including, but not limited to, a bottle, a vial, a syringe, an intravenous bag, or a tube. The kits can comprise the compounds or pharmaceutical compositions for administration to an individual in single-dose form or in multiple-dose form. The kits can further comprise instructions or a label for administering the compounds or pharmaceutical compositions to an individual according to any of the methods disclosed herein. The kits can further comprise equipment for administering the compounds or pharmaceutical compositions to an individual, including, but not limited to, needles, syringes, tubing, or intravenous bags. The kits can further comprise instructions for producing any of the compounds or pharmaceutical compositions disclosed herein.

Also provided are articles of manufacture comprising any of the compounds or conjugates described herein or compositions thereof. The articles of manufacture include suitable containers or packaging materials for the compounds or pharmaceutical compositions. The articles of manufacture include suitable containers or packaging materials for the compounds, oncolytic viruses, or pharmaceutical compositions. Examples of a suitable container include, but are not limited to, a bottle, a vial, a syringe, an intravenous bag or a tube.

The disclosure will be more fully understood by reference to the following examples. They should not, however, be construed as limiting the scope of this disclosure. It is understood that the examples and embodiments described herein are for illustrative purposes only and that various modifications or changes in light thereof will be suggested to persons skilled in the art and are to be included within the spirit and purview of this application and scope of the appended claims.

Compounds and conjugates provided herein can be prepared or synthesized according to any technique deemed suitable by the person of skill in the art. Exemplary synthetic schemes are described below.

### IX. Preparation of Compounds, Linker-Payloads, and Conjugates

Proton nuclear magnetic resonance (NMR) spectra were obtained on Bruker Ascend™ 500 MHz spectrometer. NMR spectra are reported as follows: chemical shift δ (ppm), multiplicity, coupling constant J (Hz), and integration. The abbreviations s=singlet, d =doublet, t=triplet, q=quartet, m=multiplet and br=broad are used throughout. Mass spectral data were measured using the following systems: Agilent Technologies 1290 series, Binary Pump, Diode Array Detector. Data was acquired using agilent software and purity characterized by UV wavelength 220 nm, evaporative light scattering detection (ELSD) and electrospray positive ion (ESI) (column: Agilent Poroshell 120 EC-C18, 2.7 μm, 4.6×50 mm). Solvents used: acetonitrile/water, containing 0.1% formic acid; flow rate 1 mL/min.

Abbreviations used in the examples include:

For all of the following examples, standard work-up and purification methods known to those skilled in the art can be utilized. Unless otherwise indicated, all temperatures are expressed in ° C. (degrees Centigrade). All reactions are conducted at room temperature unless otherwise noted. Synthetic methodologies illustrated herein are intended to exemplify the applicable chemistry through the use of specific examples and are not indicative of the scope of the disclosure.

As used herein, the symbols and conventions used in these processes, schemes and examples, regardless of whether a particular abbreviation is specifically defined, are consistent with those used in the contemporary scientific literature, for example, the Journal of Biological Chemistry and/or the Journal of the American Chemical Society.

For all of the following examples, standard work-up and purification methods known to those skilled in the art can be utilized. Unless otherwise indicated, all temperatures are expressed in ° C. (degrees Celsius). All methods are conducted at room temperature (“rt” or “r.t.” or “RT”), unless otherwise noted.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### EXAMPLES

### Example 1. Synthesis of Select Intermediates

Common intermediates of the STING amidobenzimidazole class of agonists examples were prepared according to the following schemes and used to make the select STING agonists compounds described herein.

### Synthesis 1A. Intermediate I-1-13

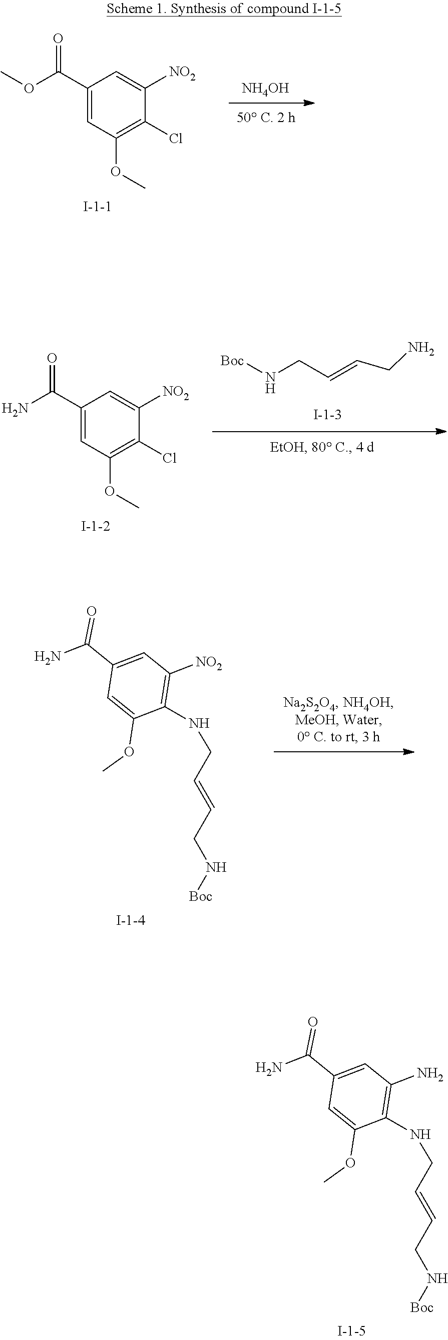 [SMILES: COC(=O)c1cc(OC)c(Cl)c([N+](=O)[O-])c1 ; "NH4OH" ; COc1cc(C(N)=O)cc([N+](=O)[O-])c1Cl ; CC(C)(C)OC(=O)NC/C=C/CN ; COc1cc(C(N)=O)cc([N+](=O)[O-])c1NC/C=C/CNC(=O)OC(C)(C)C ; COc1cc(C(N)=O)cc(N)c1NC/C=C/CNC(=O)OC(C)(C)C]

4-Chloro-3-methoxy-5-nitrobenzamide (I-1-2): Methyl 4-chloro-3-methoxy-5-nitrobenzoate (I-1-1) (40 g, 163 mmol) was stirred in NH₄OH (600 mL) at room temperature for 24 hours. The reaction temperature was then increased to 50° C. for 2 hours. An additional 120 mL of NH₄OH was added to the vessel. After an additional 2 hours of stirring at 50° C., the reaction was cooled to room temperature. The solid was filtered and rinsed with cold water. The solid was dried under vacuum to give compound I-1-2 (29 g) as a tan solid. LC-MS [ESI]: Calculated for C₈H₇ClN₂O₄[M+H⁺]: 231.01, Found: 231.1.

tert-Butyl (E)-(4-((4-carbamoyl-2-methoxy-6-nitrophenyl)amino)but-2-en-1-yl)carbamate (I-1-4): To a suspension of compound I-1-2 (30 g, 130 mmol) in EtOH (400 mL) was added (E)-tert-butyl (4-aminobut-2-en-1-yl) carbamate (I-1-3) (28 g, 150 mmol) and DIPEA (65 mL, 374 mmol). The reaction was stirred at 80° C. for 4 days and allowed to cool to room temperature. The resulting orange color solid was collected by filtration and washed with EtOH (100 mL), dried in vacuum to afford compound I-1-4 as an orange solid (27 g); LC-MS [ESI]: Calculated for C₁₇H₂₄N₄O₆ [M+H⁺]: 381.17, Found: 381.2.

tert-Butyl (E)-(4-((2-amino-4-carbamoyl-6-methoxyphenyl)amino)but-2-en-1-yl)carbamate (I-1-5): To compound I-1-4 (27 g, 71 mmol) was added methanol (480 mL). This mixture was cooled down to 0° C. After 20 minutes of stirring at 0° C., ammonium hydroxide solution (29% wt, 100 mL) was added followed by sodium hydrosulfite (75 g, 431 mmol) as a solution in water (194 mL). The flask was removed from the ice bath and stirred at room temperature. After 3 hours of stirring at room temperature, water (800 mL) was added until a clear solution was obtained. The methanol was evaporated using reduced pressure. The white solid that formed during evaporation was filtered off, washed with water twice (300 mL each), and dried under vacuum to give compound I-1-5 as a white solid (17 g). LC-MS [ESI]: Calculated for C₁₇H₂₆N₄O₄ [M+H⁺]: 351.2, Found: 351.2.

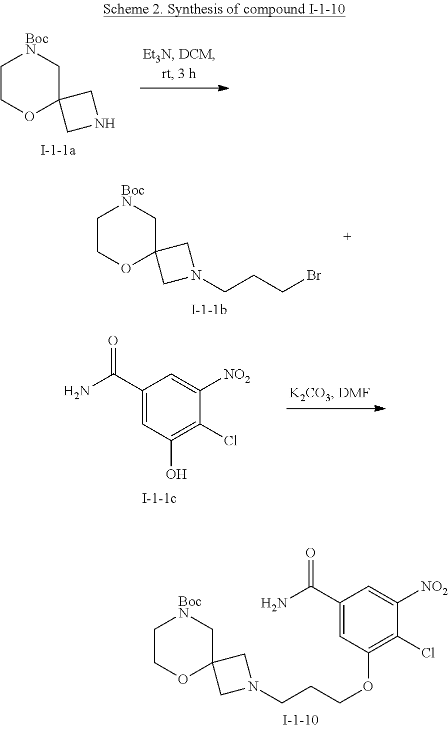 [SMILES: CC(C)(C)OC(=O)N1CCOC2(CNC2)C1 ; CC(C)(C)OC(=O)N1CCOC2(CN(CCCBr)C2)C1 ; NC(=O)c1cc(O)c(Cl)c([N+](=O)[O-])c1 ; CC(C)(C)OC(=O)N1CCOC2(CN(CCCOc3cc(C(N)=O)cc([N+](=O)[O-])c3Cl)C2)C1]

tert-Butyl 2-(3-bromopropyl)-5-oxa-2,8-diazaspiro[3,5]nonane-8-carboxylate (I-1-1b): To a stirred solution of tert-butyl 5-oxa-2,8-diazaspiro[3.5]nonane-8-carboxylate (I-1-1a, 5 g, 21.9 mmol) in DCM (45 mL) at room temperature was added 1,3-dibromopropane (2.4 mL, 24.1 mmol). The reaction mixture was stirred at room temperature for 1 hour. To this was added triethylamine (3 mL, 21.9 mmol) and the resulting suspension was stirred at room temperature for 3 hours. To this another 0.5 eq of 1,3 dibromopropane was added and the reaction was continued for another 2 hours. After which, the reaction mixture was concentrated under reduced pressure and residue was suspended in EtOAc (25 mL). Solids were filtered and washed with EtOAc (2×10 mL). The combined filtrate was concentrated under reduced pressure and the crude material was purified by column chromatography over silica gel (dry load, 40 g column, 0-20% MeOH/DCM as eluent) to afford tert-butyl 2-(3-bromopropyl)-5-oxa-2,8-diazaspiro[3.5]nonane-8-carboxylate (I-1-1b) (3.5 g, 45.7% yield) as a colorless oil. ¹H NMR (400 MHz, CDCl₃) δ 3.61-3.56 (m, 2H), 3.55 (s, 2H), 3.45 (t, J=6.7 Hz, 2H), 3.41-3.32 (m, 4H), 2.89 (d, J=7.7 Hz, 2H), 2.63 (t, J=6.8 Hz, 2H), 1.92 (p, J=6.7 Hz, 2H), 1.49 (s, 9H).

tert-Butyl 2-[3-(5-carbamoyl-2-chloro-3-nitro-phenoxy)propyl]-5-oxa-2,8-diazaspiro[3.5]nonane-8-carboxylate (I-1-10): To a stirred suspension of 4-chloro-3-hydroxy-5-nitro-benzamide (I-1-1c, 3.2 g, 15.03 mmol), and K₂CO₃ (3.46 g, 25.05 mmol) in DMF (10 mL) at room temperature was added tert-butyl 2-(3-bromopropyl)-5-oxa-2,8-diazaspiro[3.5]nonane-8-carboxylate (I-1-1b, 3.5 g, 10 mmol) and the reaction mixture was stirred at 70° C. for 16 hours. The reaction mixture was cooled to room temperature, quenched with ice cold water, and extracted with EtOAc (3×100 mL). The combined organic layer was washed with water (3×50 mL) followed by brine, dried over sodium sulfate, and concentrated under reduced pressure. The crude material was triturated with 50% EtOAc in heptane to afford tert-butyl 2-[3-(5-carbamoyl-2-chloro-3-nitro-phenoxy)propyl]-5-oxa-2,8-diazaspiro[3.5]nonane-8-carboxylate (I-1-10, 2.6 g, 5.3 mmol) as an off white solid. ¹H NMR (400 MHz, DMSO) δ 8.29 (s, 1H), 8.04 (d, J=1.7 Hz, 1H), 7.87 (d, J=1.9 Hz, 1H), 7.77 (s, 1H), 4.25 (t, J=6.2 Hz, 2H), 3.52-3.38 (m, 4H), 3.30-3.22 (m, 4H), 2.80-2.71 (m, 2H), 2.62 (t, J=6.8 Hz, 2H), 1.82 (p, J=6.6 Hz, 2H), 1.39 (s, 9H). ¹³C NMR (101 MHz, DMSO) δ 165.3, 155.5, 154.5, 149.2, 135.0, 116.9, 115.8, 115.7, 79.6, 70.9, 68.6, 62.3, 61.9, 55.8, 28.4, 27.3. LC-MS [ESI]: Calculated for C₂₁H₂₉CN₄O₇ [M+H⁺]: 485.18, Found: 485.20.

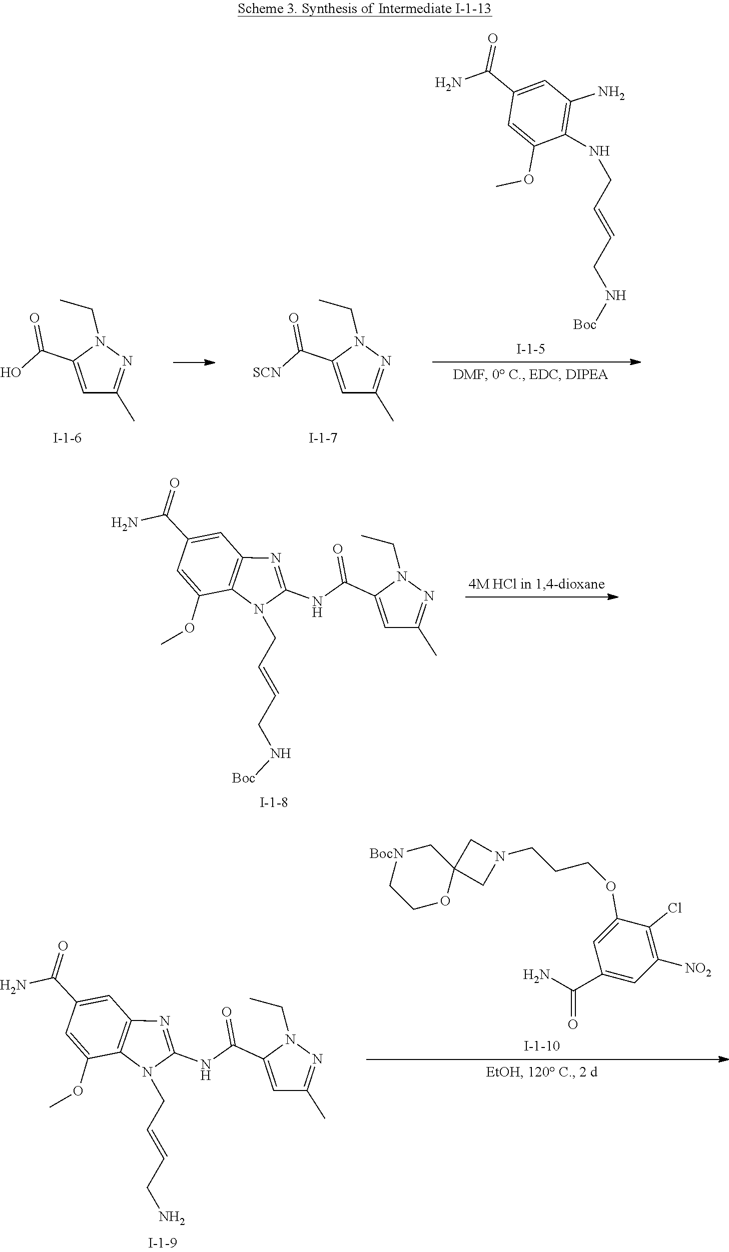

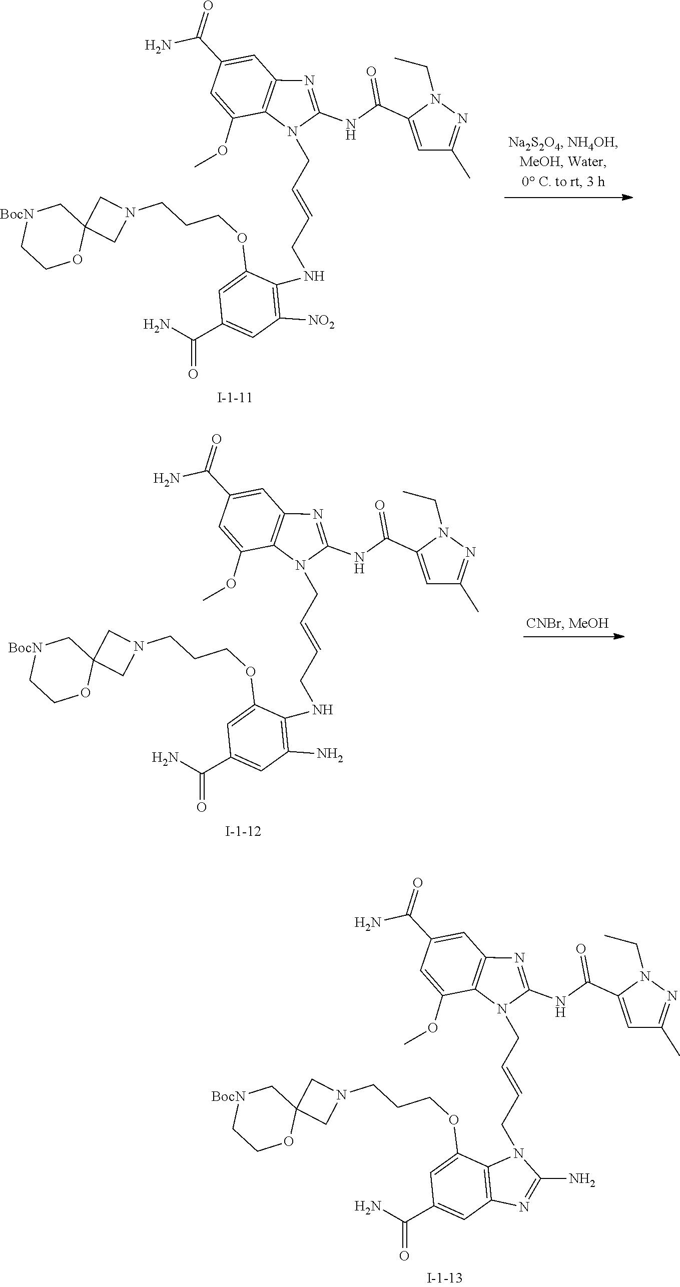 [SMILES: CCn1nc(C)cc1C(=O)Nc1nc2cc(C(N)=O)cc(OC)c2n1C/C=C/CNc1c(OCCCN2CC3(C2)CN(C(=O)OC(C)(C)C)CCO3)cc(C(N)=O)cc1[N+](=O)[O-] ; CCn1nc(C)cc1C(=O)Nc1nc2cc(C(N)=O)cc(OC)c2n1C/C=C/CNc1c(N)cc(C(N)=O)cc1OCCCN1CC2(C1)CN(C(=O)OC(C)(C)C)CCO2 ; CCn1nc(C)cc1C(=O)Nc1nc2cc(C(N)=O)cc(OC)c2n1C/C=C/Cn1c(N)nc2cc(C(N)=O)cc(OCCCN3CC4(C3)CN(C(=O)OC(C)(C)C)CCO4)c21]

1-Ethyl-3-methyl-1H-pyrazole-5-carbonyl isothiocyanate (I-1-7): To a 1 L round bottom flask was added 1-ethyl-3-methyl-1H-pyrazole-5-carboxylic acid (I-1-6, 10 g, 65 mmol) and DCM (200 mL). To this heterogeneous solution was added DMF (0.1 mL) followed by the slow addition of oxalyl chloride (7 mL, 82 mmol). After stirring for 1 hour at room temperature, the volatiles were removed under vacuum and the crude was co-evaporated twice with dichloromethane (100 mL each). Then the crude residue was dissolved in anhydrous acetone (25 mL) and added to a solution of KSCN (8.6 g, 89 mmol) in anhydrous acetone (150 mL) at 0° C. The mixture was stirred for 20 minutes. At this time, hexane (100 mL) was added to the reaction mixture and the crude heterogeneous solution was concentrated under reduced pressure to one third of the volume. The process of hexane addition and concentration was repeated twice (200 mL of hexane each). After the last concentration, hexane (200 mL) was added and the solid was removed by filtration, rinsing with hexane (100 mL). The resulting clear light-yellow filtrate was concentrated and purified by chromatography (330 g silica column; eluting with 0-20% EtOAc/hexane). The desired product eluted at 7% EtOAc/hexane. The fractions were combined and concentrated yielding compound I-1-7 (10 g) as a clear colorless liquid.

tert-Butyl (E)-(4-(5-carbamoyl-2-(1-ethyl-3-methyl-1H-pyrazole-5-carboxamido)-7-methoxy-1H-benzo[d]imidazol-1-yl)but-2-en-1-yl)carbamate (I-1-8): To a solution of compound I-1-5 (15.8 g, 45.2 mmol) in anhydrous DMF (145 mL) at 0° C. was added a solution of compound I-1-7 (7.9 g, 40.7 mmol) in dioxane (40 mL). After 20 minutes of stirring at 0° C., the formation of the intermediate thiourea was complete. Then EDC (15.87 g, 83 mmol) and DIEA (28.9 mL) were subsequently added. The reaction was warmed to room temperature and stirred overnight. To the heterogenous reaction mixture was added a mixture of 250 mL of saturated aqueous ammonium chloride and 750 mL of water. This heterogenous mixture was stirred for 1 hour at room temperature. The solid was filtered off, rinsed twice with water (200 mL each), and dried to give compound I-1-8 as a white solid (20 g). LC-MS [ESI]: Calculated for C₂₅H₃₃N₇O₅ [M+H⁺]: 512.25, Found: 512.3.

(E)-1-(4-Aminobut-2-en-1-yl)-2-(1-ethyl-3-methyl-1H-pyrazole-5-carboxamido)-7-methoxy-1H-benzo[d]imidazole-5-carboxamide (I-1-9): To a suspension of compound I-1-8 (10 g, 19.5 mmol) in 80 mL of methanol was added HCl (114 mL) as a 4M solution in 1,4-dioxane. During the HCl addition the solution went from heterogenous to homogenous with a clear yellow color. The reaction was stirred at room temperature overnight. The white solid was filtered off, rinsed with ether (200 mL), and dried in vacuum to give compound I-1-9 as an HCl salt (6 g). LC-MS [ESI]: Calculated for C₂₀H₂₅N₇O₃[M+H⁺]: 412.2, Found: 412.3.

(E)-2-(3-(5-Carbamoyl-2-((4-(5-carbamoyl-2-(1-ethyl-3-methyl-1H-pyrazole-5-carboxamido)-7-methoxy-1H-benzo[d]imidazol-1-yl)but-2-en-1-yl)amino)-3-nitrophenoxy)propyl)-5-oxa-2,8-diazaspiro[3.5]nonane-8-carboxylate (I-1-11): To a suspension of compound I-1-9 (3 g, 5.7 mmol) in EtOH (40 mL) in a pressure vessel was added compound I-1-10 (2.5 g, 5.2 mmol) and DIPEA (5 mL). The reaction was stirred at 120° C. for 2 days and allowed to cool to room temperature. The resulting precipitate was collected by filtration, washed with EtOH (20 mL) and dried under vacuum to give compound I-1-11 as a tan solid (2.3 g). LC-MS [ESI]: Calculated for C₄₁H₅₃N₁₁O₁₀ [M+H⁺]: 860.4, found: 860.4.

tert-Butyl (E)-2-(3-(3-amino-5-carbamoyl-2-((4-(5-carbamoyl-2-(1-ethyl-3-methyl-1H-pyrazole-5-carboxamido)-7-methoxy-1H-benzo[d]imidazol-1-yl)but-2-en-1-yl)amino)phenoxy)propyl)-5-oxa-2,8-diazaspiro[3.5]nonane-8-carboxylate (I-1-12): To compound I-1-11 (2.3 g, 2.7 mmol) was added methanol (50 mL). This mixture was cooled down to 0° C. After 20 minutes of stirring at 0° C., ammonium hydroxide solution (29% wt, 10 mL) was added followed by sodium hydrosulfite (3.2 g, 18.4 mmol) as a solution in water (15 mL). The flask was removed from the ice bath and stirred at room temperature. After 3 hours of stirring at room temperature, water (100 mL) was added until a clear solution was obtained. The methanol was evaporated under reduced pressure. The sticky solid that formed during evaporation was collected by decantation, washed with water twice (100 mL each), and dried under vacuum to give compound I-1-12 as a sticky solid (1.9 g). LC-MS [ESI]: Calculated for C₄₁H₅₅N₁₁O₈[M+H⁺]: 830.42, Found: 831.5.

tert-Butyl (E)-2-(3-((2-amino-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-7-yl)oxy)propyl)-5-oxa-2,8-diazaspiro[3.5]nonane-8-carboxylate (I-1-13): To a solution of compound I-1-12 (1.7 g, 2.1 mmol) in MeOH (20 mL) was added cyanogen bromide (446 mg, 4.2 mmol) at room temperature. After 5 hours, the reaction was concentrated and purified by reverse phase preparative HPLC (Phenomenex Gemini NX 5m, C18, 110 Å, 150×50 mm; Mobile phase: A) water (0.1% TFA), B) acetonitrile; Gradient: 5-45% B over 20 min, flow 50 mL/min) to give compound I-1-13 (302 mg) as a TFA salt. LC-MS [ESI]: Calculated for C₄₂H₅₄N₁₂O₈ [M+H⁺]: 855.42, Found: 855.5.

### Synthesis 1B. Intermediate I-2

Common Intermediate I-2 was synthesized as shown in Scheme 4 below.

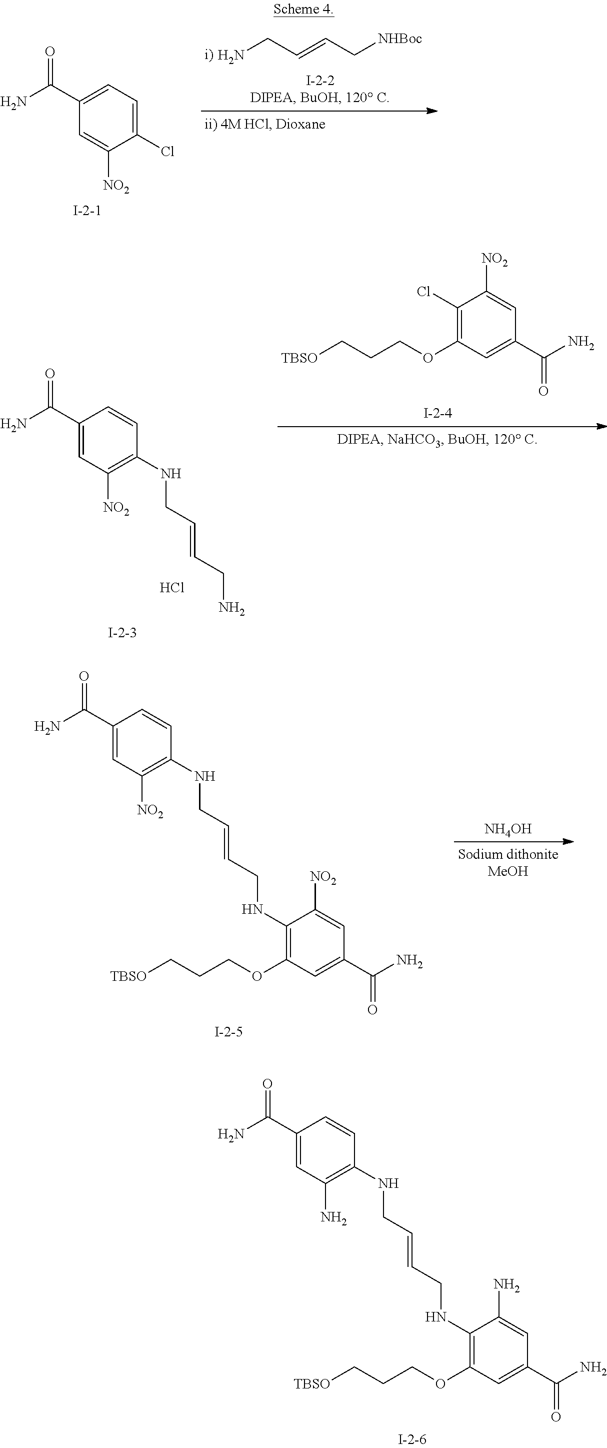

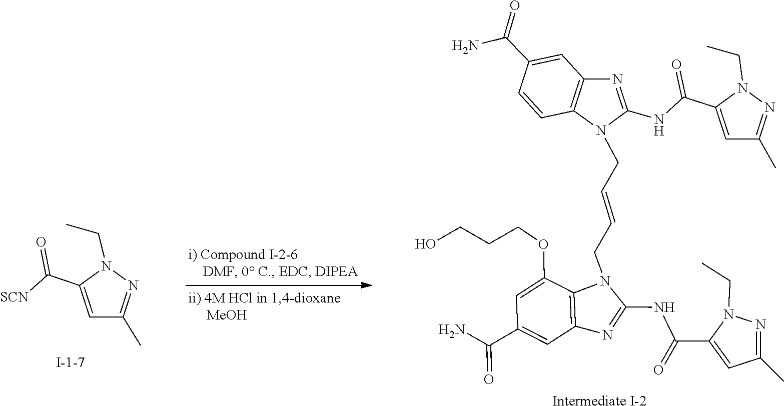

### (E)-4-((4-aminobut-2-en-1-yl)amino)-3-nitrobenzamide (I-2-3)

To a suspension of compound I-2-1 (40 g, 200 mmol) in EtOH (500 mL) was added (E)-tert-butyl (4-aminobut-2-en-1-yl) carbamate (I-2-2) (44.6 g, 240 mmol) and DIEA (105 mL, 604 mmol). The reaction was stirred at 80° C. for 4 days and allowed to cool to room temperature. The resulting orange solid was collected by filtration and washed with EtOH (100 mL) to give boc-protected compound I-2-3 as an orange solid (70 g, >95% pure), LC-MS [ESI]: Calculated for C₁₆H₂₃N₄O₅ [M+H⁺]: 351.16, Found: 351.36.

To a suspension of boc-protected compound I-2-3 (68 g, 194 mmol) in MeOH (160 mL) was added HCl (4M in 1,4 dioxane, 230 mL). The reaction mixture was stirred at room temperature for 2 hours. Then the mixture was evaporated under reduced pressure and dried under vacuum to give the compound I-2-3 (64 g, >95% pure) as an HCl salt. LC-MS [ESI]: Calculated for C₁₁H₁₅N₄O₃ [M+H⁺]: 251.11, Found: 251.23.

### (E)-3-(3-((tert-butyldimethylsilyl)oxy)propoxy)-4-((4-((4-carbamoyl-2-nitrophenyl)amino)but-2-en-1-yl)amino)-5-nitrobenzamide (I-2-5)

To a suspension of compound I-2-3 (HCl salt, 10 g, 31 mmol) in anhydrous iPrOH (50 mL) in a pressure vessel was added compound I-2-4 (12 g, 31 mmol) and DIEA (27 mL). The reaction was stirred at 120° C. for 7 days and allowed to cool to room temperature. The solvent was evaporated under vacuum, and the resulting residue was dissolved in acetonitrile (250 mL) at 60° C. The solution was cooled with an ice bath, and the resulting precipitate was collected by filtration, washed with ice cold acetonitrile (20 mL) and dried under vacuum to give crude compound I-2-5 as a red solid (about 80% pure, 11 g). Repeated on the same scale 3 more times due to a limited volume of the available pressure vessel. LC-MS [ESI]: Calculated for C₂₇H₃₉N₆O₈Si [M+H⁺]: 603.25, Found: 603.48.

### Preparation of 4-chloro-3-[3-(1-methyl-1-trimethylsilyl-ethoxy)propoxy]-5-nitro-benzamide (I-2-4)

In a sealed tube flask, was taken 4-chloro-3-hydroxy-5-nitro-benzamide (I-1-1c (18.5 g, 85 mmol) and K₂CO₃ (23.6 g, 170 mmol) in DMF (150 mL). To this was added [1-(3-bromopropoxy)-1-methyl-ethyl]-trimethyl-silane (28.2 g, 110.5 mmol). The vessel was capped, and the resulting suspension was heated to 100° C. for 2 hours followed by at room temperature for 16 hours. Reaction mixture poured onto ice cold water and filtered. Solids were washed with water followed by hexanes and dried under reduced pressure to afford 4-chloro-3-[3-(1-methyl-1-trimethylsilyl-ethoxy)propoxy]-5-nitro-benzamide I-2-4 (28 g, 84%) as light yellow solid. ¹H NMR (400 MHz, DMSO) δ 8.30 (s, 1H), 8.05 (d, J=1.7 Hz, 1H), 7.89 (d, J=1.8 Hz, 1H), 7.78 (s, 1H), 4.30 (t, J=5.9 Hz, 2H), 3.80 (t, J=6.1 Hz, 2H), 1.98 (p, J=6.0 Hz, 2H), 0.84 (s, 10H), 0.06 (s, 6H). LC-MS [ESI]: Calculated for C₁₆H₂₆ClN₂O₅Si [M+H⁺]: 389.13, Found: 389.20.

### (E)-3-amino-4-((4-((2-amino-4-carbamoylphenyl)amino)but-2-en-1-yl)amino)-5-(3-((tert-butyldimethylsilyl)oxy)propoxy)benzamide (I-2-6)

To crude compound I-2-5 (˜80% pure, 43 g, 71.4 mmol) was added methanol (400 mL) to make a suspension. This mixture was cooled down to 0° C. After 20 minutes of stirring at 0° C., ammonium hydroxide solution (29% wt, 150 mL) was added followed by water (250 mL) and sodium hydrosulfite (74 g, 425 mmol). The flask was removed from the ice bath and stirred at room temperature. After 2 hours of stirring at room temperature, the insoluble material was filtered off and the filtrate was extracted with DCM (2×800 mL). DCM solution was washed with brine, then dried over Na₂SO₄, filtered, and evaporated under reduced pressure to give a dark residue. This residue was dissolved in DCM/MeOH mixture (9:1, 20 mL) and purified by silicagel column (330 g silicagel, DCM/MeOH (10% to 20%) to give compound I-2-6 as a tan solid (15 g, yield 38%, >90% pure). LC-MS [ESI]: Calculated for C₂₇H₄₃N₆O₄Si [M+H⁺]: 543.31, Found: 543.4.

### 1-ethyl-3-methyl-1H-pyrazole-5-carbonyl isothiocyanate (I-1-7)

To a 1 L round bottom flask was added 1-ethyl-3-methyl-1H-pyrazole-5-carboxylic acid (20 g, 130 mmol) and DCM (400 mL). To this heterogeneous solution was added DMF (0.2 mL) followed by the slow addition of oxalyl chloride (14 mL, 164 mmol). After stirring for 1 hour at room temperature, the volatiles were removed under vacuum and the crude was co-evaporated twice with dichloromethane (200 mL each). Then the crude residue was dissolved in anhydrous acetone (50 mL) and added to a solution of KSCN (17.2 g, 178 mmol) in anhydrous acetone (300 mL) at 0° C. The mixture was stirred for 20 minutes. At this time, hexane (200 mL) was added to the reaction mixture and the crude heterogeneous solution was concentrated in vacuo to one third of the volume. The process of hexane addition and concentration was repeated twice (300 mL of hexane each). After the last concentration, hexane (400 mL) was added and the solid was removed by filtration, rinsing with hexane (100 mL). The resulting clear brown filtrate was concentrated and purified by chromatography (330 g silica column; eluting with 0-20% EtOAc/hexane). The desired product elutes at 7% EtOAc/hexane. The fractions were combined and concentrated yielding compound I-1-7 (20 g) as a clear colorless liquid. LC-MS [ESI]: Calculated for C₈H₁₀N₃OS [M+H⁺]: 196.06, Found: 196.1.

### (E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-hydroxypropoxy)-1H-benzo[d]imidazole-5-carboxamide (Intermediate I-2)

To a solution of compound I-2-6 (13 g, 24 mmol) in anhydrous DMF (60 mL) at 0° C. was added a solution of compound I-1-7 (14 g, 72 mmol). After 3 hours of stirring at room temperature, the formation of the intermediate thiourea was complete. Then EDC (18 g, 94 mmol) and DIEA (17 mL) were sequentially added. The reaction was stirred at room temperature for 24 hours. The reaction mixture was concentrated under reduced pressure to 50 mL to give mostly DMF solution of crude compound, LC-MS [ESI]: Calculated for C₄₃H₅₇N₁₂O₆Si [M+H⁺]: 865.42, Found: 865.6. To this crude solution was added acetonitrile (75 mL), water (75 mL) and TFA (8 mL). The resulting mixture was stirred for 16 hours at room temperature. The solid was filtered off, rinsed twice with acetonitrile/water mixture (1:1, 120 mL each), and dried to give compound Intermediate I-2 as a tan solid (TFA salt, 11 g, >95% pure). ¹H NMR (400 MHz, DMSO) δ 12.75 (bs, 1H), 7.98-7.96 (m, 3H), 7.73-7.71 (d, J =8 Hz, 1H), 7.64 (s, 1H), 7.42 (d, J=8 Hz, 1H), 7.34-7.32 (m, 3H), 6.54 (d, J=12.8 Hz, 2H), 5.98 (m, 1H), 5.75 (m, 1H), 4.94 (d, J=4.4 Hz, 2H), 4.82 (d, J=4.4 Hz, 2H), 4.52 (q, J=6.8 Hz), 3.47 (t, J=6.0 Hz, 2H), 2.11 (m, 6H), 1.75 (m, 2H), 1.26 (m, 6H) LC-MS [ESI]: Calculated for C₃₇H₄₃N₁₂O₆ [M+H⁺]: 751.34, Found: 751.3.

### Synthesis 1C. Intermediate I-3

Common Intermediate I-3 was synthesized as shown in Scheme 5 below.

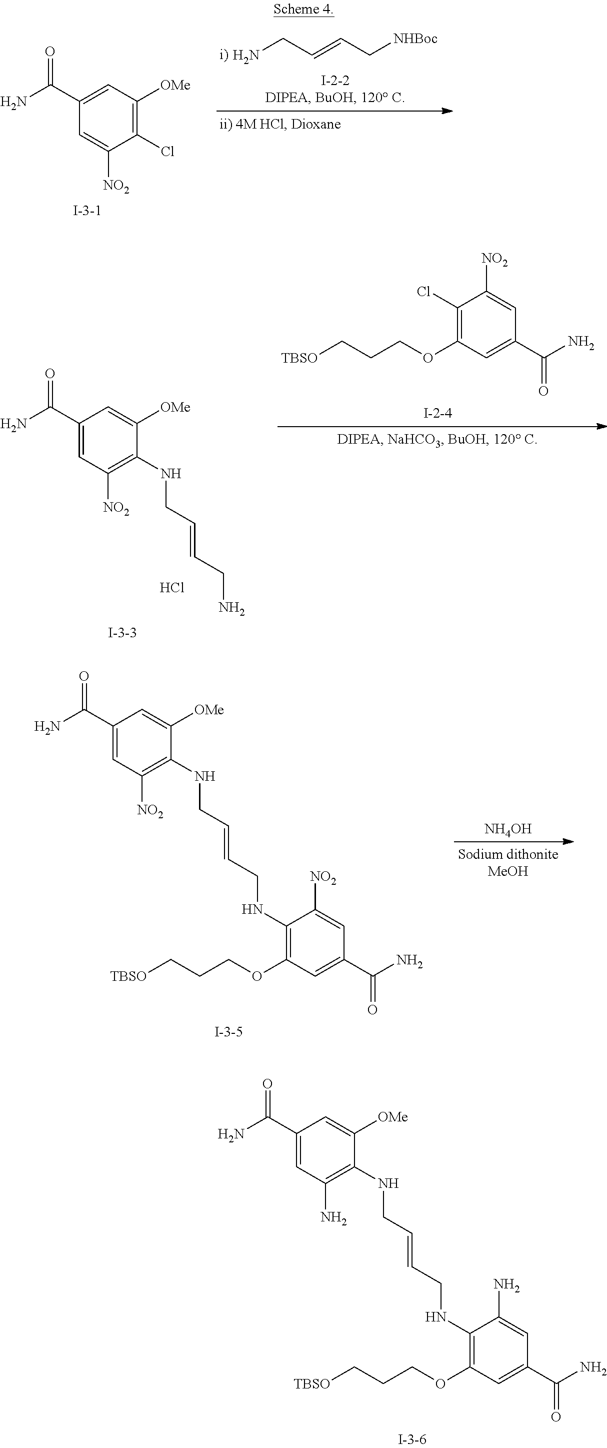

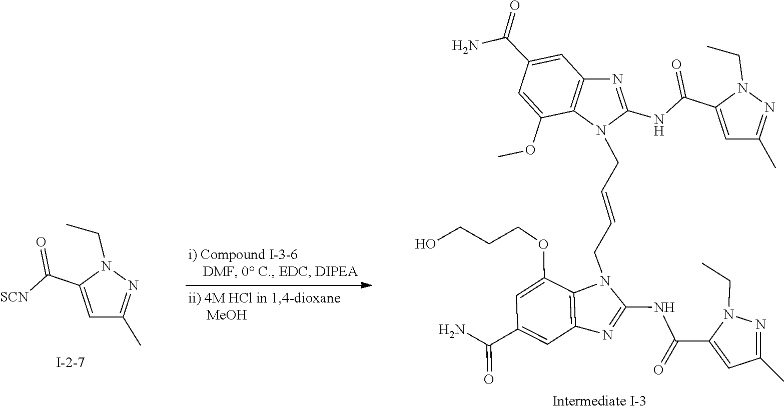

### Preparation of 4-chloro-3-methoxy-5-nitrobenzamide (I-3-1)

Methyl 4-chloro-3-methoxy-5-nitrobenzoate (CAS #63603-09-8) (40 g, 163 mmol) was stirred in NH₄OH (600 mL) at room temperature for 24 hours. The reaction temperature was then increased to 50° C. for 2 hours. An additional 120 mL of NH₄OH was added to the vessel. After an additional 2 hours of stirring at 50° C., the reaction was cooled to room temperature. The solid was filtered and rinsed with cold water. The solid was dried under vacuum to give compound I-3-1 (29 g) as a tan solid. LC-MS [ESI]: Calculated for C₈H₇ClN₂O₄[M+H⁺]: 231.01, Found: 231.1; ¹H NMR (400 MHz, Methanol-d₄): δ 7.93 (d, J=2 Hz, 1H), 7.82 (d, J=2 Hz, 1H), 4.04 (s, 3H).

### Preparation of (E)-4-((4-aminobut-2-en-1-yl)amino)-3-methoxy-5-nitrobenzamide hydrochloride (I-3-3)

To a suspension of compound I-3-1 (30 g, 130 mmol) in EtOH (400 mL) was added (E)-tert-butyl (4-aminobut-2-en-1-yl) carbamate I-2-2 (28 g, 150 mmol) and DIPEA (65 mL, 374 mmol). The reaction was stirred at 80° C. for 4 days and allowed to cool to room temperature. The resulting orange color solid was collected by filtration and washed with EtOH (100 mL), dried in vacuum to afford compound Boc protected I-3-3 as an orange solid (27 g); ¹H NMR (400 MHz, DMSO-d₆): δ 8.18 (d, J=1.6 Hz, 1H), 8.00 (br s, 1H), 7.75 (t, J=6 Hz, 1H), 7.55 (d, J=1.2 Hz, 1H), 7.31 (br s, 1H), 6.92 (br s, 1H), 5.53 (br s, 2H), 4.09 (d, J=5.2 Hz, 2H), 3.87 (s, 3H), 3.47 (br s, 2H), 1.37 (s, 9H); LC-MS [ESI]: Calculated for C₁₇H₂₄N₄O₆[M+H⁺]: 381.17, Found: 381.2.

A solution of above prepared tert-butyl (E)-(4-((4-carbamoyl-2-methoxy-6-nitrophenyl)amino)but-2-en-1-yl)carbamate (31 g, 81.542 mmol, 1 equiv.) in DCM (373 mL) was cooled to 0° C. 4N Hydrogen chloride in 1,4-dioxane (157.571 mL, 4321.751 mmol, 53 equiv.) was added and then reaction mixture was brought to room temperature and stir it for 1 hour. Reaction progress was monitored by TLC. Solvent was evaporated to afford desired (E)-4-((4-aminobut-2-en-1-yl)amino)-3-methoxy-5-nitrobenzamide hydrochloride (I-3-3) (25 g, 79.091 mmol, 96.9% yield) as a red solid. MS (ESI) m/z [M+H]⁺(%): 281.35 (90.6).

### Preparation of (E)-3-(3-((tert-butyldimethylsilyl)oxy)propoxy)-4-((4-((4-carbamoyl-2-methoxy-6-nitrophenyl)amino)but-2-en-1-yl)amino)-5-nitrobenzamide (I-3-5)

To a suspension of compound I-3-3 (12 g, 34 mmol) in anhydrous EtOH (40 mL) in a pressure vessel was added compound I-2-4 (11 g, 28 mmol) and DIEA (30 mL). The reaction was stirred at 120° C. for 3 days and allowed to cool to room temperature. The resulting precipitate was collected by filtration, washed with EtOH (20 mL) and dried to give crude compound I-3-5 as a red solid (about 80% pure, 4.4 g). The crude material was taken to the next step. ¹H NMR (400 MHz, DMSO-d₆): δ 8.18-8.16 (m, 2H), 8.02 (br s, 2H), 7.73-7.67 (m, 2H), 7.53 (m, 2H), 7.32 (br s, 2H), 5.61 (d, J=4.4 Hz, 2H), 4.12-4.06 (m, 6H), 3.84 (s, 3H), 3.75 (t, J=6 Hz, 2H), 1.94 (p, J=6 Hz, 2H), 0.83 (s, 9H), 0.01 (s, 6H); LC-MS [ESI]: Calculated for C₂₈H₄₀N₆O₉Si [M+H⁺]: 633.26, Found: 633.3.

### Preparation of (E)-3-amino-4-((4-((2-amino-4-carbamoyl-6-methoxyphenyl)amino)but-2-en-1-yl)amino)-5-(3-((tert-butyldimethylsilyl)oxy)propoxy)benzamide (I-3-6)

To crude compound I-3-5 (8.8 g, 13.9 mmol) was added methanol (500 mL). This mixture was cooled down to 0° C. After 20 minutes of stirring at 0° C., ammonium hydroxide solution (29% wt, 30 mL) was added followed by sodium hydrosulfite (24 g, 431 mmol) as a solution in water (150 mL). The flask was removed from the ice bath and stirred at room temperature. After 3 hours of stirring at room temperature, water (100 mL) was added until a clear solution was obtained. The methanol was evaporated using reduced pressure. The dark tar, formed during evaporation, was extracted with DCM twice (300 mL each). DCM solution was washed with brine, then dried over Na₂SO₄, filtered, and evaporated under vacuum to give compound I-3-6 as a tan solid (3.3 g). ¹H NMR (400 MHz, DMSO-d₆): δ 7.60 (br s, 2H), 6.96 (br s, 2H), 6.85-6.84 (m, 2H), 6.78-6.76 (m, 2H), 5.67-5.65 (m, 2H), 4.64-4.62 (m, 4H), 3.99 (t, J=6 Hz, 2H) 3.77-3.73 (m, 7H), 3.65-3.45 (m, 4H), 1.89 (p, J=6 Hz, 2H), 0.83 (s, 9H), 0.02 (s, 6H).

### Preparation of (E)-1-(4-(5-carbamoyl-2-(1-ethyl-3-methyl-1H-pyrazole-5-carboxamido)-7-(3-hydroxypropoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide (I-3)

To a solution of compound I-3-6 (1.4 g, 2.5 mmol) in anhydrous DMF (10 mL) at 0° C. was added a solution of compound I-2-7 (1.5 g, 7.6 mmol) in dioxane (4 mL). After 5 hours of stirring at 0° C., the formation of the intermediate thiourea was complete. Then EDC (1.5 g, 7.8 mmol) and DIEA (1.7 mL) were added. The reaction was warmed to room temperature and stirred overnight. The mixture was concentrated under vacuum to 10 mL to give mostly DMF solution of crude compound OTBS protected compound I-3. To this solution was added acetonitrile (10 mL), water (10 mL) and TFA (1 mL). The resulting mixture was stirred for 4 hours at room temperature. The solid was filtered off, rinsed twice with water (20 mL each), and dried to give compound I-3 as a tan solid (1.0 g). ¹H NMR (400 MHz, DMSO-d₆): δ 12.81 (br s, 2H), 8.0-7.92 (m, 2H), 7.64 (d, J=2.8 Hz, 2H), 7.39-7.32 (m, 4H), 6.53-6.51 (m, 2H), 5.85-5.81 (m, 2H), 4.95-4.87 (m, 4H) 4.57-4.46 (m, 4H), 4.05 (t, J=6.4 Hz, 2H), 3.70 (s, 3H), 2.10 (s, 3H), 2.09 (s, 3H), 1.70 (p, J=6.4 Hz, 2H), 1.31-1.22 (m, 6H); LC-MS [ESI]: Calculated for C₃₈H₄₄N₁₂O₇[M+H⁺]: 781.35, Found: 781.3.

### Synthesis 1D. Intermediate I-4

Common Intermediate I-4 was synthesized as shown in Scheme 6 below.

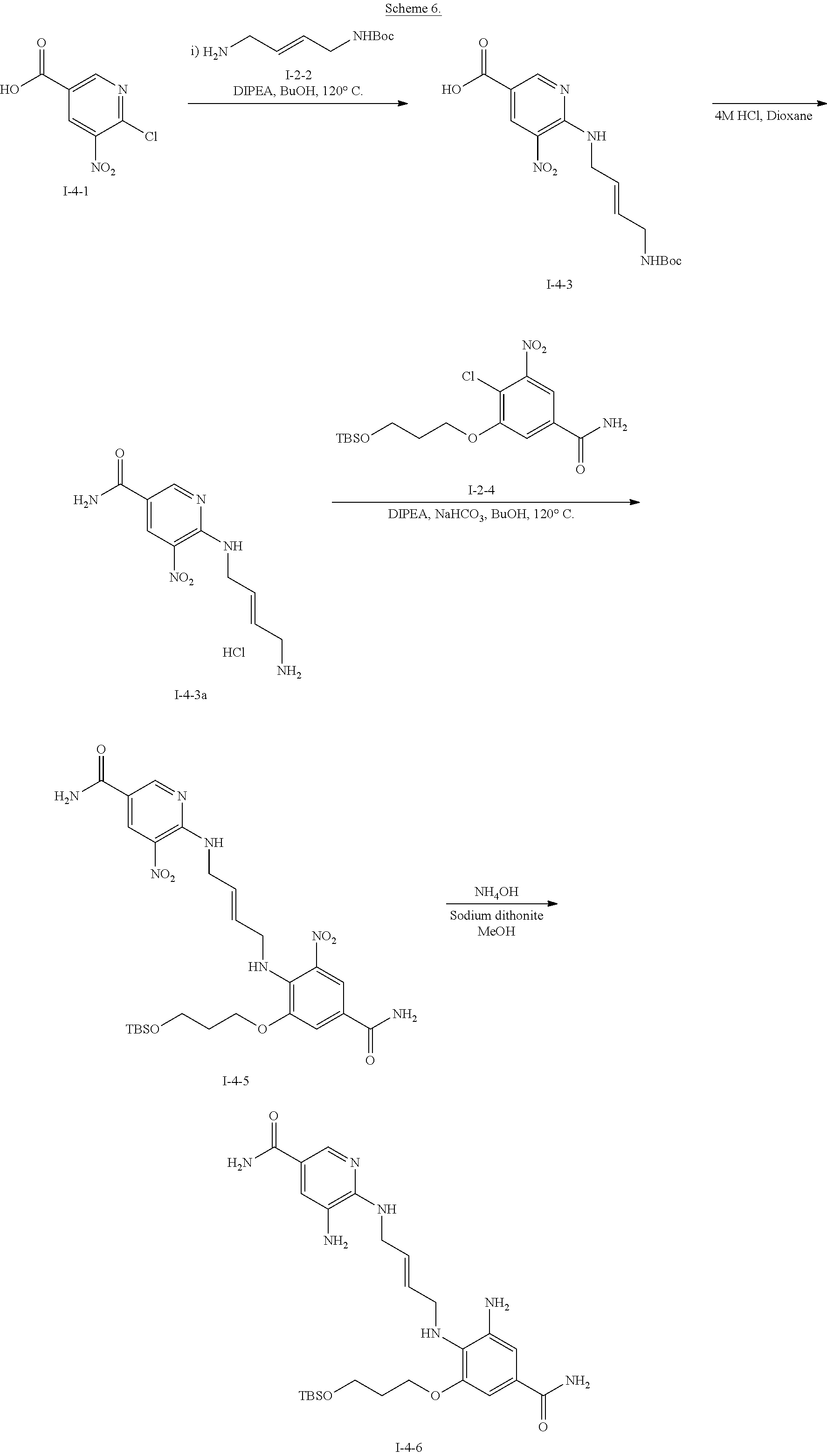

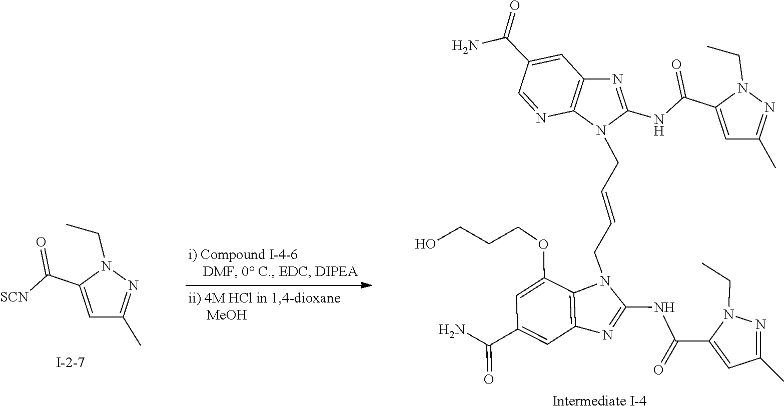

(E)-6-((4-((tert-butoxycarbonyl)amino)but-2-en-1-yl)amino)-5-nitronicotinic acid (I-4-3): To a stirred solution of 6-chloro-5-nitronicotinic acid (I-4-1, 100 g, 494 mmol) in butanol (1 L) was added (E)-tert-butyl (4-aminobut-2-en-1-yl)carbamate (I-2-2, 92 g, 494 mmol) and DIPEA (128 g, 988 mmol). The mixture was heated to 120° C. and stirred for 16 hours. The reaction mixture was concentrated under reduced pressure. The compound was dissolved in DCM (1.5 L) and water (1.5 L), and adjusted pH to about 6-7 by adding aq. 1M HCl, concentrated to remove DCM, and the remainder was filtered and washed with water (100 mL) and dried under vacuum to afford compound I-4-3 as a yellow solid (78 g, 45%). LC-MS [ESI]: Calculated for C₁₅H₂₀N₄O₆ [M+H⁺]: 353.14, Found: 354.2.

(E)-6-((4-aminobut-2-en-1-yl)amino)-5-nitronicotinamide (I-4-3a): To a stirred solution of compound I-4-3, 78 g, 222 mmol) (780 mL) in 1,4-dioxane (60 mL), 4M HCl 1,4-dioxane (60 mL) was added and the reaction mixture was stirred 16 hours at room temperature. The mixture was concentrated under reduced pressure, the remaining was dissolved in water (60 mL) and adjusted the pH to about 10 by adding saturated sodium carbonate solution and the mixture was stirred for 4 hours, and filtered and dried to give compound I-4-3a (42 g, 90% yield) as a yellow solid. LC-MS [ESI]: Calculated for C₁₀H₁₃N₅O₃ [M+H⁺]: 252.1, Found: 252.1.

(E)-6-((4-((2-(3-((tert-butyldimethylsilyl)oxy)propoxy)-4-carbamoyl-6-nitrophenyl)amino)but-2-en-1-yl)amino)-5-nitronicotinamide (I-4-5): To a stirred solution of compound I-2-4 (30 g, 0.077 mol), compound I-4-3a (29 g, 0.116 mol) and Na₂CO₃ (16.4 g, 0.154 mol) in n-butanol (750 mL) was added and the mixture was heated to 120° C. for 16 hours. The mixture was cooled to room temperature, DCM (750 mL) was added, the reaction was stirred for 30 minutes, and filtered to remove solids. The filtrate was concentrated under reduced pressure, and purified by silica gel column chromatography (DCM:MeOH=30:1) to give compound I-4-5 (19 g, 40% yield) as a red solid. LC-MS [ESI]: Calculated for C₂₆H₃₇N₇O₈Si [M+H⁺]: 604.25, Found: 604.3.

(E)-5-amino-6-((4-((2-amino-6-(3-((tert-butyldimethylsilyl)oxy)propoxy)-4-carbamoylphenyl)amino)but-2-en-1-yl)amino)nicotinamide (I-4-6): A stirred solution of compound I-4-5 (19 g, 0.031 mol) and aqueous ammonia (25% v/v, 66.5 mL, 3.9 mol) in methanol (760 mL) was cooled to 0° C. and then a solution of Na₂S₂O₄ (54 g, 0.314 mol) in water (156 mL) was added slowly, The reaction mixture was allowed to warm to room temperature and stirred for 20 minutes. Then the mixture was filtered, and the filtrate was concentrated under reduce pressure at 20° C. The crude compound was purified by silica gel column chromatography (DCM:MeOH=65:35) to give compound I-4-6 (4.2 g, 24.5% yield) as a grey color solid. LC-MS [ESI]: Calculated for C₂₆H₄₁N₇O₄Si [M+H⁺]: 544.3, Found: 544.35.

Intermediate I-4: To a stirred solution of compound I-4-6 (200 mg, 0.36 mmol) in anhydrous DMF (10 mL) at 0° C. was added 1-ethyl-3-methyl-1H-pyrazole-5-carbonyl isothiocyanate (I-2-7, 2 eq) and the reaction mixture was stirred for 1 hour at room temperature and then slowly warmed to 40° C., after which DCC (227 mg, 1.1 mmol) was added and the mixture stirred for 16 hours at 40° C. The reaction mixture was purified by reverse phase preparative IPLC to give compound OTBS-protected Intermediate I-4 (100 mg). To a solution of OTBS protected Intermediate I-4 (100 mg) in MeOH (3 mL) was added a solution of HCl in 1,4 dioxane (4M, 1.5 mL) at 0° C. The reaction was stirred for 4 hours at room temperature. The reaction was concentrated under reduced pressure. The pH of the residue was adjusted to about 7-8 by adding LiOH solution (1M), and then the solution was concentrated. The residue was purification by purified by reverse phase preparative HPLC to give Intermediate I-4 (60 mg) as a solid. LC-MS [ESI]: Calculated for C₃₆H₄₁N₁₃O₆ [M+H⁺]: 752.33, Found: 752.4

### Synthesis 1E. Intermediate I-5

Common Intermediate I-5 was synthesized as shown in Scheme 7 below.

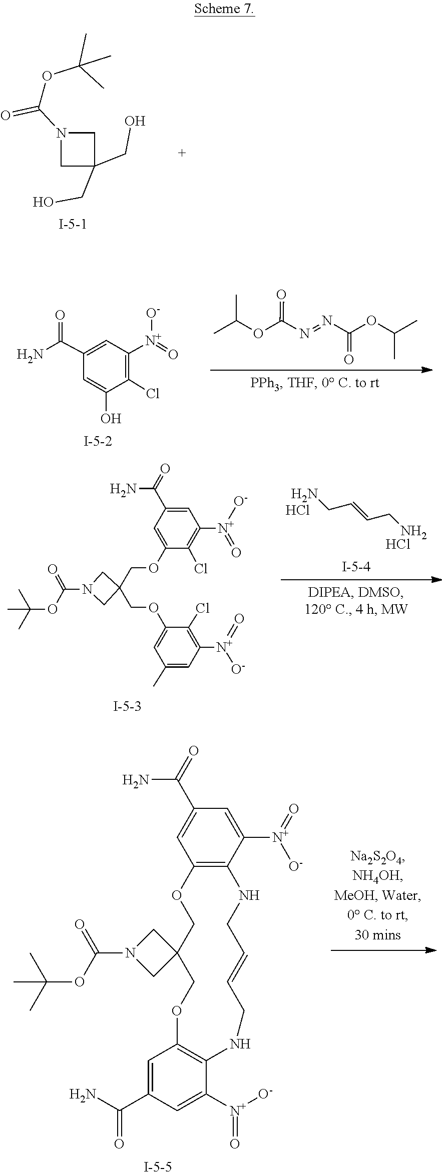

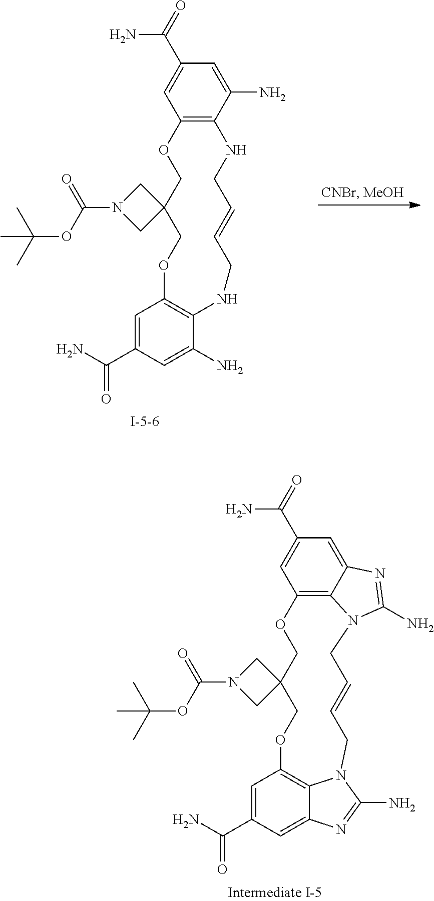 [SMILES: CC(C)(C)OC(=O)N1CC2(COc3cc(C(N)=O)cc(N)c3NC/C=C/CNc3c(N)cc(C(N)=O)cc3OC2)C1 ; CC(C)(C)OC(=O)N1CC2(COc3cc(C(N)=O)cc4nc(N)n(c34)C/C=C/Cn3c(N)nc4cc(C(N)=O)cc(c43)OC2)C1]

tert-Butyl 3,3-bis[(5-carbamoyl-2-chloro-3-nitro-phenoxy)methyl]azetidine-1-carboxylate (I-5-3): To a stirred solution of tert-butyl 3,3-bis(hydroxymethyl)azetidine-1-carboxylate (I-5-1, 500.0 mg, 2.3 mmol), 4-chloro-3-hydroxy-5-nitro-benzamide (I-1-1c, 996.9 mg, 4.6 mmol) and triphenylphosphine (1811 mg, 6.9 mmol) in THF (20 mL) at 0° C. was added dropwise isopropyl (E)-N-isopropoxycarbonyliminocarbamate (1.36 mL, 6.9 mmol). The reaction mixture was allowed to warm to room temperature and stirred for 16 hours. The reaction mixture was concentrated under reduced pressure. Crude material was purified by silica gel column chromatography (solid load, 24 g cartridge, 20-100% EA/hexanes as eluent). Pure fractions were combined and concentrated to give tert-butyl 3,3-bis[(5-carbamoyl-2-chloro-3-nitro-phenoxy)methyl]azetidine-1-carboxylate (I-5-3) (401 mg, 0.65 mmol, 28.36% yield) as white solid. ¹H NMR (400 MHz, DMSO) δ 8.28 (s, 2H), 8.10 (d, J=1.8 Hz, 2H), 7.97 (d, J=1.9 Hz, 2H), 7.81 (s, 2H), 4.53 (s, 4H), 3.94 (s, 4H), 1.40 (s, 9H). LC-MS [ESI]: Calculated for C₂₄H₂₆Cl₂N₄O₉ [M+H⁺]: 585.11, Found: 585.2.

tert-Butyl (15E)-9,22-dicarbamoyl-11,20-dinitro-spiro[2,6-dioxa-13,18-diazatricyclo[17.4.0.07,12]tricosa-1 (23), 7,9,11,15,19,21-heptaene-4,3′-azetidine]-1′-carboxylate (I-5-5): In a 20 mL vial, a suspension of tert-butyl 3,3-bis[(5-carbamoyl-2-chloro-3-nitro-phenoxy)methyl]azetidine-1-carboxylate (I-5-3) (460 mg, 0.75 mmol), DIPEA (0.63 mL, 4.49 mmol) and (E)-but-2-ene-1,4-diamine dihydrochloride (I-5-4, 238.1 mg, 1.5 mmol) in DMSO (7.5 mL) was irradiated at normal absorption level at 120° C. for 4 hours. The crude material was mixed with additional previously obtained crude material purified by prep-HPLC (Column: Phenomenex kinetex 5 μm C₁₈ 100 Å, 250×50 mm and using 10-70% ACN: 10 mM NH4OAc in water as eluent, Flow rate: 118 mL/min, run time: 30 minutes to afford tert-butyl (15E)-9,22-dicarbamoyl-11,20-dinitro-spiro[2,6-dioxa-13,18-diazatricyclo[17.4.0.07,12]tricosa-1 (23), 7,9,11,15,19,21-heptaene-4,3′-azetidine]-1′-carboxylate (I-5-5) (200 mg, 0.32 mmol, 42.5% yield) as a brown solid. ¹H NMR (400 MHz, DMSO) δ 8.30 (d, J=1.9 Hz, 2H), 8.03 (s, 2H), 7.82 (d, J=2.1 Hz, 2H), 7.78 (t, J=6.6 Hz, 2H), 7.39 (s, 2H), 5.68 (d, J=1.8 Hz, 2H), 4.25 (s, 4H), 4.07 (d, J=6.5 Hz, 4H), 3.91 (s, 4H), 1.41 (s, 9H). LC-MS [ESI]: Calculated for C₂₈H₃₃₃N₇O₁₀ [M+H⁺]: 628.23, Found: 628.20.

tert-Butyl (15E)-11,20-diamino-9,22-dicarbamoyl-spiro[2,6-dioxa-13,18-diazatricyclo[17.4.0.07,12]tricosa-1 (19), 7 (12), 8,10,15,20,22-heptaene-4,3′-azetidine]-1′-carboxylate (I-5-6): To a stirred suspension of tert-butyl (15E)-9,22-dicarbamoyl-11,20-dinitro-spiro[2,6-dioxa-13,18-diazatricyclo[17.4.0.07,12]tricosa-1 (19), 7 (12), 8,10,15,20,22-heptaene-4,3′-azetidine]-1′-carboxylate (I-5-5, 100 mg, 0.16 mmol) in methanol (5 mL) at 0° C. was added sodium hydrosulfite (277.4 mg, 1.59 mmol) in H₂O (1 mL) followed by the simultaneous addition of ammonium hydroxide (1 mL, 7.9 mmol). The resulting suspension was allowed to warm to room temperature and stirred at room temperature for 2 hours. LCMS showed the reaction was complete. The reaction mixture was filtered, and solids were washed with MeOH. Combined filtrate was concentrated and purified by reverse phase preparative HPLC (Column: Phenomenex kinetex 5 μm C₁₈ 100 Å, 250×21.2 mm and using 10-50% ACN: 10 mM NH4OAc in water as eluent, Flow rate: 21.2 mL/min, run time: 30 minutes to give tert-butyl (15E)-11,20-diamino-9,22-dicarbamoyl-spiro[2,6-dioxa-13,18-diazatricyclo[17.4.0.07,12]tricosa-1 (19), 7 (12), 8,10,15,20,22-heptaene-4,3′-azetidine]-1′-carboxylate (I-5-6) (21 mg, 0.03 mmol, 23.2% yield) as a white solid. ¹H NMR (400 MHz, DMSO) δ 7.62 (s, 2H), 7.11-6.93 (m, 4H), 6.90 (d, J=1.8 Hz, 2H), 5.38 (t, J=3.0 Hz, 2H), 4.69 (s, 4H), 4.33 (s, 4H), 3.86 (d, J=28.7 Hz, 6H), 3.46 (s, 4H), 1.41 (s, 9H). LC-MS [ESI]: Calculated for C₂₈H₃₇N₇O₆[M+H⁺]: 568.28, Found: 568.20.

tert-Butyl (3E)-7,25-diamino-11,21-dicarbamoyl-spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (Intermediate I-5): To a stirred solution of tert-butyl (15E)-11,20-diamino-9,22-dicarbamoyl-spiro[2,6-dioxa-13,18-diazatricyclo[17.4.0.07,12]tricosa-1 (19), 7 (12), 8,10,15,20,22-heptaene-4,3′-azetidine]-1′-carboxylate (I-5-6, 20 mg, 0.04 mmol) in methanol (1 mL) at room temperature was added cyanogen bromide (30 mg, 0.28 mmol). The resulting solution was stirred at this temperature for 2 hours. MeOH was concentrated under reduced pressure, residue was diluted with MTBE, suspension was filtered, and solids were washed with MTBE and dried under reduced pressure to afford tert-butyl (3E)-7,25-diamino-11,21-dicarbamoyl-spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (Intermediate I-5) (17 mg, 0.0248 mmol, 70.305% yield) as a light yellow solid. ¹H NMR (400 MHz, DMSO) δ 12.98 (s, 2H), 8.65 (s, 3H), 8.10 (s, 2H), 7.86 (s, 2H), 7.67-7.37 (m, 4H), 5.38 (s, 2H), 4.71 (s, 4H), 4.52 (s, 4H), 3.86 (s, 4H), 1.44 (s, 9H). LC-MS [ESI]: Calculated for C₃₀H₃₅N₉O₆[M+H⁺]: 618.28, Found: 618.40.

### Synthesis 1F. Intermediate I-6

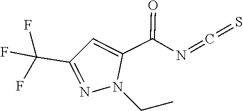 [SMILES: CCn1nc(C(F)(F)F)cc1C(=O)N=C=S]

To a mixture of 1-ethyl-3-(trifluoromethyl)-1H-pyrazole-5-carboxylic acid (130 mg, 0.62 mmol) in anhydrous DCM (3 mL) was added a solution of oxalyl chloride in DCM (2.0M, 0.47 mL, 0.94 mmol) dropwise. The reaction mixture was stirred at room temperature for 1 hour. The reaction mixture was concentrated, and the residue was re-dissolved in DCM and re-concentrated to afford crude 1-ethyl-3-(trifluoromethyl)pyrazole-5-carbonyl chloride, which was dissolved in acetone (6 mL) and added to a mixture of potassium thiocyanate (121 mg, 1.25 mmol) in acetone (4 mL) at 0° C. The reaction mixture was stirred at room temperature for 1 hour, and then filtered. The filtrate was concentrated. The residue was purified by silica chromatography eluted with 0-10% EA in heptane to afford 1-ethyl-3-(trifluoromethyl)pyrazole-5-carbonyl isothiocyanate (I-6, 122 mg, 78%). LC-MS [ESI]: Calculated for C₈H₇F₃N₃OS [M+H⁺]: 250.02, Found: 250.2.

### Example 2. Synthesis of Select STING Agonists

### Synthesis 2A. Compound 1

Compound 1 was synthesized as shown in Scheme 8 below.

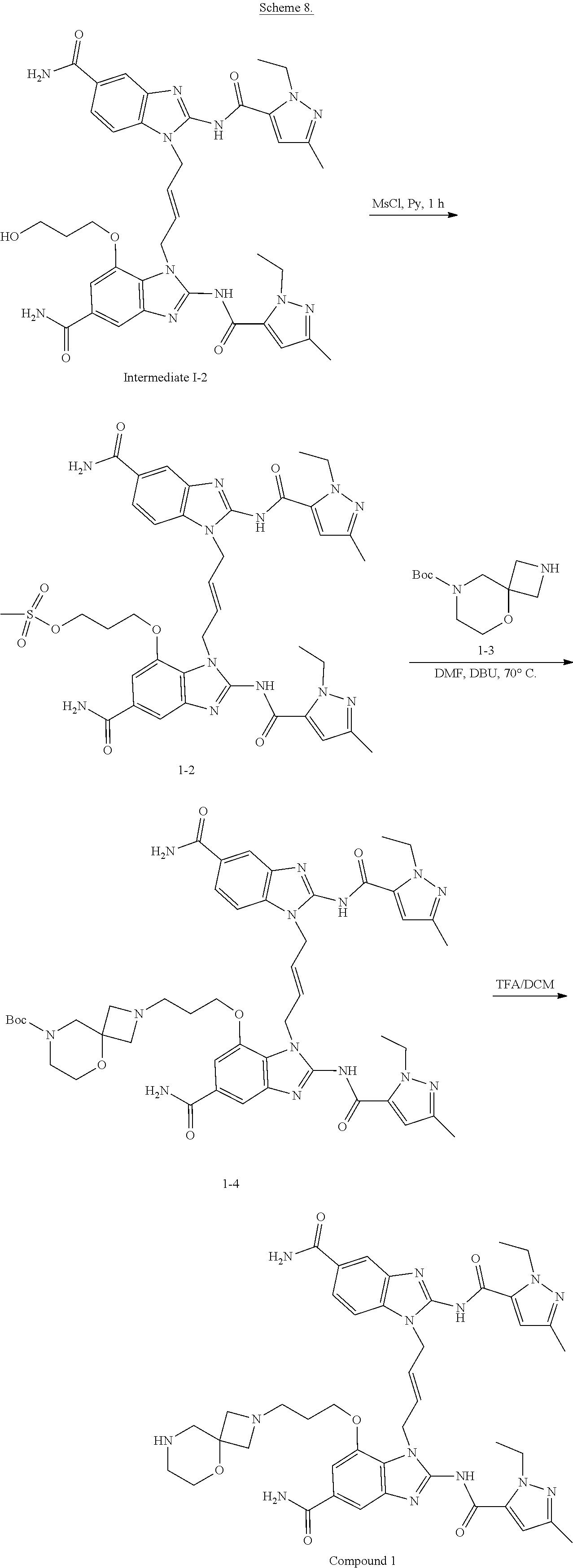

Compound 1-2: To a cooled solution (ice bath) of Intermediate I-2 (800 mg, 1.07 mmol) in anhydrous pyridine (10 mL) was added methanesulfonyl chloride (0.66 mL, 8.6 mmol) dropwise. The mixture was stirred at room temperature for a period of 1 hour. Then the mixture was concentrated under reduced pressure and the crude material was purified by reverse phase preparative HPLC to give compound I-2 as a white solid after lyophilization (502 mg); LC-MS [ESI]: Calculated for C₃₈H₄₄N₁₂O₈S [M+H⁺]: 829.31, Found: 829.4.

Compound 1-4: The compound I-2 (500 mg, 0.6 mmol)) was dissolved in anhydrous DMF (4 mL). To this solution, the diamine 1-3 (275 mg, 1.2 mmol) was added, followed by DBU (0.9 mmol, 0.13 mL), and the mixture was stirred at 70° C. for 16 hours. The crude mixture was purified directly by reverse phase preparative HPLC to give compound I-4 as a white powder after lyophilization (280 mg); LC-MS [ESI]: Calculated for C₄₈H₆₀N₁₄O₈ [M+H⁺]: 961.47, Found: 961.5.

Compound 1: The compound I-4 (60 mg, 62.5 mmol) was treated with TFA/DCM (1/4, v/v, 2 mL) at room temperature for 10 minutes. The reaction mixture was evaporated to dryness under reduced pressure and the residue was purified by reverse phase preparative HPLC to give Compound 1 as a white powder (44 mg, TFA salt); LC-MS [ESI]: Calculated for C₄₃H₅₂N₁₄O₆[M+H⁺]: 861.42, Found: 861.55; ¹H NMR (400 MHz, DMSO) δ 12.83 (s, 2H), 10.64 (s, 1H), 10.37 (s, 1H), 9.25 (s, 2H), 7.96 (d, J=8.1 Hz, 2H), 7.70 (dd, J=8.3, 1.7 Hz, 1H), 7.66 (s, 1H), 7.38 (d, J=8.4 Hz, 2H), 7.33 (s, 1H), 6.54 (s, 1H), 6.50 (s, 1H), 5.96 (d, J=15.2 Hz, 1H), 5.68 (d, J=15.5 Hz, 1H), 4.94 (d, J=4.5 Hz, 2H), 4.81 (d, J=6.2 Hz, 2H), 4.57-4.46 (m, 5H), 4.39 (d, J=9.9 Hz, 2H), 4.08 (s, 10H), 3.98 (s, 3H), 3.89 (s, 2H), 3.79 (s, 2H), 3.36 (s, 2H), 3.11 (s, 2H), 2.12 (d, J=3.8 Hz, 6H), 1.85 (d, J=31.3 Hz, 2H), 1.26 (t, J=7.1 Hz, 6H).

### Synthesis 2B. Compound 2

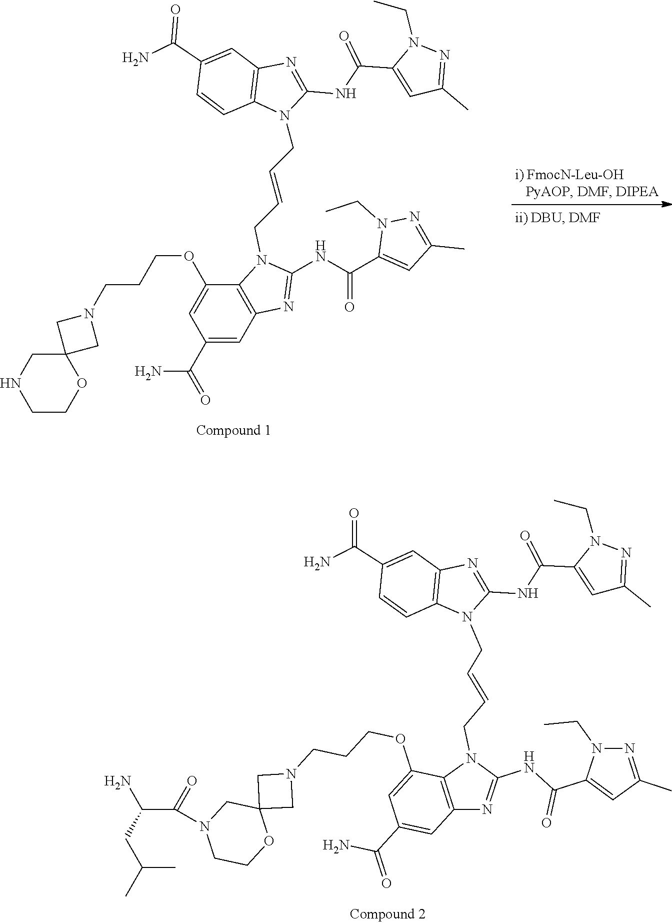 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(CNCCO4)C3)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@@H](N)CC(C)C)CCO4)c21]

To a solution of Compound 1 (TFA salt, 20 mg) and Fmoc-Leu-OH (7 mg, 20 mmol) in DMF (1 mL) was added PyAOP (10.4 mg, 20 mmol), followed by DIPEA (20 mL). After 10 min, DBU (40 mL) was added, and the mixture was stirred at room temperature for 20 minutes. The crude reaction was purified directly by reverse phase preparative HPLC to give Compound 2 (14 mg, TFA salt) as a white powder. LC-MS [ESI]: Calculated for C₄₉H₆₃N₁₅O₇[M+H⁺]: 974.5, Found: 974.7; ¹H NMR (400 MHz, DMSO) δ 12.83 (s, 2H), 8.18 (s, 1H), 8.13 (s, 2H), 7.97 (s, 2H), 7.73-7.64 (m, 2H), 7.39 (t, J=7.0 Hz, 3H), 7.32 (s, 1H), 6.53 (d, J=12.0 Hz, 2H), 5.71 (s, 1H), 4.94 (s, 2H), 4.81 (d, J=5.6 Hz, 2H), 4.53 (d, J=7.2 Hz, 4H), 4.36 (s, 1H), 4.07 (s, 4H), 3.93 (d, J=13.5 Hz, 1H), 3.46 (s, 1H), 3.40 (s, 1H), 2.12 (s, 6H), 1.83 (s, 2H), 1.70 (s, 1H), 1.56 (s, 1H), 1.49-1.39 (m, 1H), 1.27 (m, 6H), 0.95-0.84 (m, 6H).

### Synthesis 2C. Compound 3

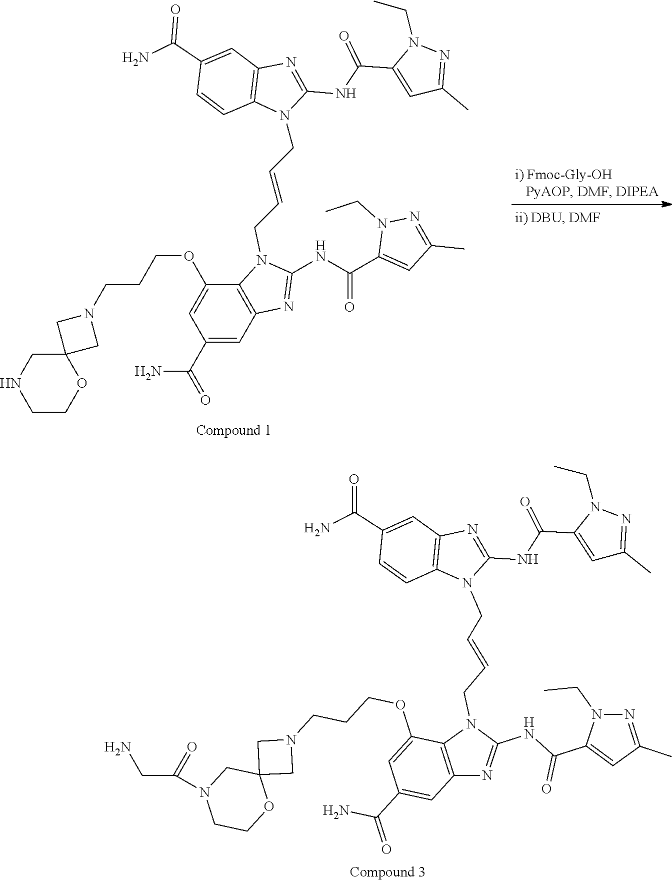 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(CNCCO4)C3)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)CN)CCO4)c21]

Compound 3 was synthesized using the same methods in an analogous fashion as described herein. Calculated for C₄₅H₅₅N₁₅O₇[M+H⁺]: 918.44, Found: 918.6.

### Synthesis 2D. Compound 4

Compound 4 was synthesized as shown in Scheme 9 below.

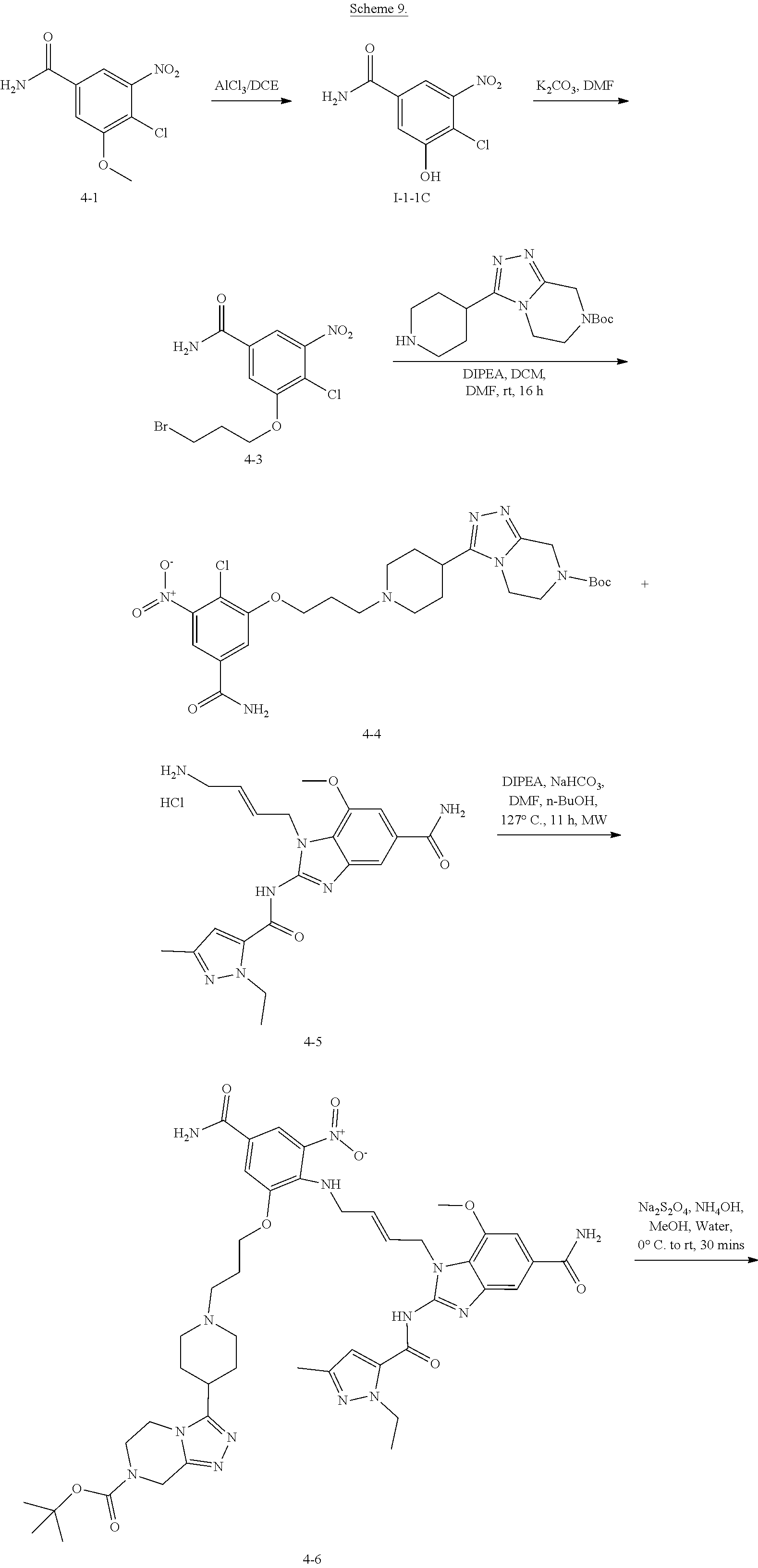

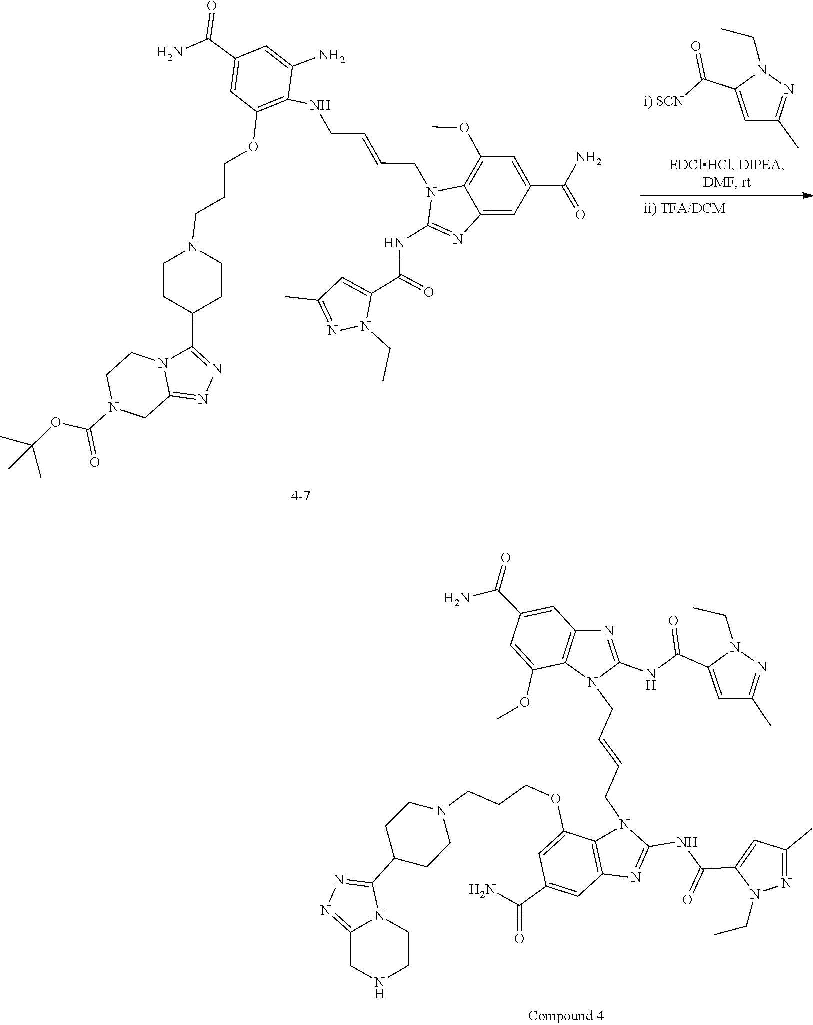

4-Chloro-3-hydroxy-5-nitro-benzamide (I-1-1c): In a round bottomed flask, was taken 4-chloro-3-methoxy-5-nitro-benzamide (4-1) (25 g, 108.41 mmol). To this was added DCE (250 mL). To the resulting suspension at room temperature was added AiCl₃ (43.4 g, 325.22 mmol) portion wise. The suspension turned clear and the color changed to dark brown. The reaction mixture was stirred at room temperature for 16 hours. Crushed ice was added to the reaction mixture followed by the addition of water. The reaction mixture was allowed to stir at room temperature for 2 hours and the brown gum turned into an off-white suspension. Solids were filtered, washed with water followed by hexanes, and dried under reduced pressure to afford 4-chloro-3-hydroxy-5-nitro-benzamide 4-2 (21 g, 89.4% yield) as a light brown solid. ¹H NMR (400 MHz, DMSO) δ 11.57 (s, 1H), 8.19 (s, 1H), 7.92 (d, J=2.0 Hz, 1H), 7.73 (d, J=2.0 Hz, 1H), 7.67 (s, 1H). LC-MS [ESI]: Calculated for C₇H5ClN₂O₄[M+H⁺]: 217.0, Found: 217.0.

3-(3-Bromopropoxy)-4-chloro-5-nitro-benzamide (4-3): To a stirred solution of 4-chloro-3-hydroxy-5-nitro-benzamide (4-2, 1.5 g, 6.93 mmol) in DMF (10 mL) at rt was added K₂CO₃ (23.9 g, 17.31 mmol) followed by the addition of 1,3-dibromopropane (1.41 mL, 13.85 mmol). The suspension was heated to 60° C. and stirred for 2 hours. Reaction mixture was cooled to room temperature, cold water was added to the reaction mixture, solids were filtered, washed with water followed by hexanes, and dried under reduced pressure to afford 3-(3-bromopropoxy)-4-chloro-5-nitro-benzamide (4-3, 2.2 g, 94.1% yield) as an off-white solid. Crude compound (4-3) was used in the next step without further purification. LC-MS [ESI]: Calculated for C₁₀H₁₀BrClN₂O₄[M+H⁺]: 337.0, Found: 337.0.

tert-Butyl 3-[1-[3-(5-carbamoyl-2-chloro-3-nitro-phenoxy)propyl]-4-piperidyl]-6,8-dihydro-5H-[1,2,4]triazolo[4,3-a]pyrazine-7-carboxylate (4-4): To a stirred solution of tert-butyl 3-(4-piperidyl)-6,8-dihydro-5H-[1,2,4]triazolo[4,3-a]pyrazine-7-carboxylate; hydrochloride (2.1 g, 6.11 mmol) in DCM (25 mL) at room temperature was added DIPEA (2.34 mL, 13.44 mmol) followed by the addition of 3-(3-bromopropoxy)-4-chloro-5-nitro-benzamide (4-3, 2.27 g, 6.72 mmol). The suspension was stirred at room temperature for 30 minutes. To this was added DMF (15 mL) and the suspension was stirred at room temperature for 16 hours The reaction was diluted with water and extracted with DCM (3×100 mL). Emulsion was observed, and the biphasic layer was passed through a plug of celite. The two layers were separated, and the combined organic part was dried over sodium sulfate before concentrating to dryness. The crude was then triturated with MTBE to afford tert-butyl 3-[1-[3-(5-carbamoyl-2-chloro-3-nitro-phenoxy)propyl]-4-piperidyl]-6,8-dihydro-5H-[1,2,4]triazolo[4,3-a]pyrazine-7-carboxylate (4-4, 1.3 g, 37.7% yield) as an off white solid. ¹H NMR (400 MHz, DMSO) δ 8.31 (s, 1H), 8.05 (d, J=1.7 Hz, 1H), 7.89 (d, J=1.9 Hz, 1H), 7.79 (s, 1H), 4.63 (s, 2H), 4.29 (t, J=6.2 Hz, 2H), 3.96 (t, J=5.5 Hz, 2H), 3.74 (t, J=5.4 Hz, 2H), 2.94 (d, J=32.3 Hz, 2H), 2.73 (d, J=6.7 Hz, 1H), 2.10-1.61 (m, 8H), 1.44 (s, 9H). LC-MS [ESI]: Calculated for C₂₅H₃₄ClN₇O₆ [M+H⁺]: 564.23, Found: 564.20.

tert-Butyl 3-[1-[3-[5-carbanoyl-2-[[(E)-4-[5-carbamoyl-2-[(2-ethyl-5-methyl-pyrazole-3-carbonyl)amino]-7-methoxy-benzimidazol-1-yl]but-2-enyl]amino]-3-nitro-phenoxy]propyl]-4-piperidyl]-6,8-dihydro-5H-[1,2,4]triazolo[4,3-a]pyrazine-7-carboxylate (4-6): A stirred suspension of tert-butyl 3-[1-[3-(5-carbamoyl-2-chloro-3-nitro-phenoxy)propyl]-4-piperidyl]-6,8-dihydro-5H-[1,2,4]triazolo[4,3-a]pyrazine-7-carboxylate (4-4, 631.6 mg, 1.12 mmol), 1-[(E)-4-aminobut-2-enyl]-2-[(2-ethyl-5-methyl-pyrazole-3-carbonyl)amino]-7-methoxy-benzimidazole-5-carboxamide hydrochloride (I-1-9, 501.6 mg, 1.12 mmol), sodium bicarbonate (188 mg, 2.24 mmol) and DIPEA (0.78 mL, 4.48 mmol) in 1-butanol (10 mL) was heated to 127° C. under MW irradiation (at normal absorption level) for 11 hours. LC-MS showed major desired product formation. Residue was suspended in DMF and filtered. Filtrate was purified by reverse phase preparative HPLC (Column: Phenomenex kinetex 5 μm C₁₈ 100 Å, 250×50 mm and using 5-40% ACN: 1% TFA in water as eluent, Flow rate: 118.1 mL/min, run time: 25 mins to afford tert-butyl 3-[1-[3-[5-carbamoyl-2-[[(E)-4-[5-carbamoyl-2-[(2-ethyl-5-methyl-pyrazole-3-carbonyl)amino]-7-methoxy-benzimidazol-1-yl]but-2-enyl]amino]-3-nitro-phenoxy]propyl]-4-piperidyl]-6,8-dihydro-5H-[1,2,4]triazolo[4,3-a]pyrazine-7-carboxylate (4-6, 320 mg, 0.34 mmol, 30.4% yield) as a white solid. ¹H NMR (400 MHz, DMSO) δ 9.32 (s, 1H), 8.16 (dd, J=13.6, 1.8 Hz, 1H), 8.01 (s, 1H), 7.66 (dd, J=4.8, 1.3 Hz, 1H), 7.50 (d, J=2.0 Hz, 1H), 7.44-7.29 (m, 3H), 6.60 (d, J=3.7 Hz, 1H), 5.79 (dt, J=15.7, 5.6 Hz, 1H), 5.67 (dt, J=15.6, 5.4 Hz, 1H), 4.90 (d, J=5.6 Hz, 3H), 4.69 (s, 4H), 4.57 (q, J=7.1 Hz, 3H), 4.14 (d, J=5.8 Hz, 3H), 3.99 (ddt, J=18.1, 12.4, 6.3 Hz, 5H), 3.86 (d, J=2.2 Hz, 3H), 3.79 (t, J=5.5 Hz, 2H), 3.55 (d, J=11.8 Hz, 2H), 3.23-3.09 (m, 2H), 3.00 (dt, J=21.7, 7.8 Hz, 3H), 2.16 (s, 4H), 2.12-2.03 (m, 3H), 2.02-1.88 (m, 2H), 1.45 (d, J=2.4 Hz, 9H), 1.32 (td, J=7.1, 2.0 Hz, 3H). LC-MS [ESI]: Calculated for C₄₅H₅₈N₁₄O₉ [M+H⁺]: 939.46, Found: 939.50.

tert-Butyl 3-[1-[3-[3-amino-5-carbanoyl-2-[[(E)-4-[5-carbamoyl-2-[(2-ethyl-5-methyl-pyrazole-3-carbonyl)amino]-7-methoxy-benzimidazol-1-yl]but-2-enyl]amino]phenoxy]propyl]-4-piperidyl]-6,8-dihydro-5H-[1,2,4]triazolo[4,3-a]pyrazine-7-carboxylate (4-7): To a stirred suspension of tert-butyl 3-[1-[3-[5-carbamoyl-2-[[(E)-4-[5-carbamoyl-2-[(2-ethyl-5-methyl-pyrazole-3-carbonyl)amino]-7-methoxy-benzimidazol-1-yl]but-2-enyl]amino]-3-nitro-phenoxy]propyl]-4-piperidyl]-6,8-dihydro-5H-[1,2,4]triazolo[4,3-a]pyrazine-7-carboxylate (4-6) (100 mg, 0.11 mmol) in methanol (3 mL) at 0° C. was added sodium hydrosulfite (185.4 mg, 1.06 mmol) in water (1 mL) followed by the simultaneous addition of ammonium hydroxide (0.69 mL, 5.32 mmol). The resulting suspension was allowed to warm to room temperature and stirred for 2 hours. LCMS showed the reaction was completed. Reaction mixture was concentrated under reduced pressure before it was suspended in DMF and filtered through a plug. Filtrate was purified by reverse phase preparative HPLC (Column: Phenomenex kinetex 5 μm C₁₈ 100 Å, 250×21.2 mm and using 5-40% ACN: 0.1% TFA in water as eluent, Flow rate: 21.2 mL/min, run time: 25 mins to afford tert-butyl 3-[1-[3-[3-amino-5-carbamoyl-2-[[(E)-4-[5-carbamoyl-2-[(2-ethyl-5-methyl-pyrazole-3-carbonyl)amino]-7-methoxy-benzimidazol-1-yl]but-2-enyl]amino]phenoxy]propyl]-4-piperidyl]-6,8-dihydro-5H-[1,2,4]triazolo[4,3-a]pyrazine-7-carboxylate (4-7) (59 mg, 0.06 mmol, 60.9% yield) as a white solid. ¹H NMR (400 MHz, MeOD) δ 7.67 (d, J=1.4 Hz, 1H), 7.39 (dd, J=3.8, 1.6 Hz, 1H), 6.94 (d, J=1.7 Hz, 1H), 6.69 (s, 1H), 6.65 (d, J=1.8 Hz, 1H), 6.04 (dt, J=15.4, 5.1 Hz, 1H), 5.48 (dt, J=14.6, 6.8 Hz, 1H), 5.02-4.96 (m, 2H), 4.66 (q, J=7.1 Hz, 2H), 4.12 (s, 2H), 4.02-3.97 (m, 2H), 3.97-3.93 (m, 2H), 3.92 (s, 2H), 3.89-3.79 (m, 2H), 3.23 (d, J=16.3 Hz, 2H), 2.27 (d, J=5.6 Hz, 5H), 2.09-1.99 (m, 2H), 1.53 (d, J=3.1 Hz, 9H), 1.41 (t, J=7.1 Hz, 3H). LC-MS [ESI]: Calculated for C₄₅H₆₀N₁₄O₇[M+H⁺]: 909.40, Found: 909.40.

tert-Butyl 3-[1-[3-[6-carbamoyl-3-[(E)4-[5-carbamoyl-22-[(2-ethyl-5-methyl-pyrazole-: 3-carbonyl)amino]-7-methoxy-benzimidazol-1-yl]but-2-enyl]-2-[(2-ethyl-5-methyl-pyrazole-3-carbonyl)amino]benzimidazol-4-yl]oxypropyl]-4-piperidyl]-6,8-dihydro-5-[1,2,4]triazolo[4,3-a]pyrazine-7-carboxylate: To a stirred solution of tert-butyl 3-[1-[3-[3-amino-5-carbamoyl-2-[[(E)-4-[5-carbamoyl-2-[(2-ethyl-5-methyl-pyrazole-3-carbonyl)amino]-7-methoxy-benzimidazol-1-yl]but-2-enyl]amino]phenoxy]propyl]-4-piperidyl]-6,8-dihydro-5H-[1,2,4]triazolo[4,3-a]pyrazine-7-carboxylate (4-7) (25 mg, 0.03 mmol) in DMF (1 mL) at room temperature was added DIPEA (0.02 mL, 0.14 mmol) followed by the addition of 2-ethyl-5-methyl-pyrazole-3-carbonyl isothiocyanate (I-1-7, 5.91 mg, 0.03 mmol). The resulting yellow solution was stirred at room temperature for 2 hours followed by at 40° C. for 1 hour. The reaction mixture was cooled to room temperature and to this was added N,N′-dicyclohexylmethanediimine (5.6 mg, 0.03 mmol). The reaction mixture was warmed to 40° C. and stirred at this temperature for 16 hours. Reaction mixture was cooled to room temperature and purified by reverse phase preparative HPLC (Column: Phenomenex kinetex 5 μm C₁₈ 100 Å, 250×21.2 mm and using 5-70% ACN: 0.1% TFA in water as eluent, Flow rate: 21.2 mL/min, run time: 30 mins to afford tert-butyl 3-[1-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-[(2-ethyl-5-methyl-pyrazole-3-carbonyl)amino]benzimidazol-4-yl]oxypropyl]-4-piperidyl]-6,8-dihydro-5H-[1,2,4]triazolo[4,3-a]pyrazine-7-carboxylate (7 mg, 0.0065 mmol, 23.784% yield) as a white solid. LC-MS [ESI]: Calculated for C₅₃H₆₇H₁₇O₈ [M+H⁺]: 1070.54, Found: 1070.60.

1-[(E)-4-[5-Carbamoyl-2-[(2-ethyl-5-methyl-pyrazole-3-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]but-2-enyl]-2-[(2-ethyl-5-methyl-pyrazole-3-carbonyl)amino]-7-methoxy-benzimidazole-5-carboxamide (Compound 4): To a stirred solution of tert-butyl 3-[1-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-[(2-ethyl-5-methyl-pyrazole-3-carbonyl)amino]benzimidazol-4-yl]oxypropyl]-4-piperidyl]-6,8-dihydro-5H-[1,2,4]triazolo[4,3-a]pyrazine-7-carboxylate (7 mg, 0.01 mmol) in HFIP (0.5 mL) at room temperature was added 5% TFA in HFIP (1. mL, 0.65 mmol). The solution was stirred at room temperature for 2 hours. Reaction mixture was concentrated under reduced pressure before it was taken in DMF and purified by reverse phase preparative HPLC (Column: Phenomenex kinetex 5 μm C₁₈ 100 Å, 250×21.2 mm and using 5-35% ACN: 0.1% TFA in water as eluent, Flow rate: 21.2 mL/min, run time: 25 mins to afford 1-[(E)-4-[5-carbamoyl-2-[(2-ethyl-5-methyl-pyrazole-3-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]but-2-enyl]-2-[(2-ethyl-5-methyl-pyrazole-3-carbonyl)amino]-7-methoxy-benzimidazole-5-carboxamide (Compound 4, 3 mg, 47.28% yield) as a white solid (di TFA salt). ¹H NMR (400 MHz, DMSO) δ 12.89 (s, 2H), 9.38 (s, 2H), 8.00 (d, J=20.8 Hz, 2H), 7.70-7.62 (m, 2H), 7.40 (s, 2H), 7.33 (d, J=11.0 Hz, 2H), 6.54 (d, J=4.6 Hz, 2H), 5.90-5.70 (m, 3H), 4.92 (d, J=15.3 Hz, 4H), 4.53 (d, J=13.8 Hz, 6H), 4.29-3.94 (m, 6H), 3.69 (d, J=30.5 Hz, 7H), 3.18-2.84 (m, 7H), 2.12 (d, J=10.5 Hz, 7H), 1.94 (d, J=13.0 Hz, 5H), 1.29 (dt, J=11.2, 7.1 Hz, 6H). LC-MS [ESI]: Calculated for C₄₈H₅₉N₁₇O₆ [M+H⁺]: 970.50, Found: 970.50.

### Synthesis 2E. Compound 5

Compound 5 was synthesized as shown in Scheme 10 below.

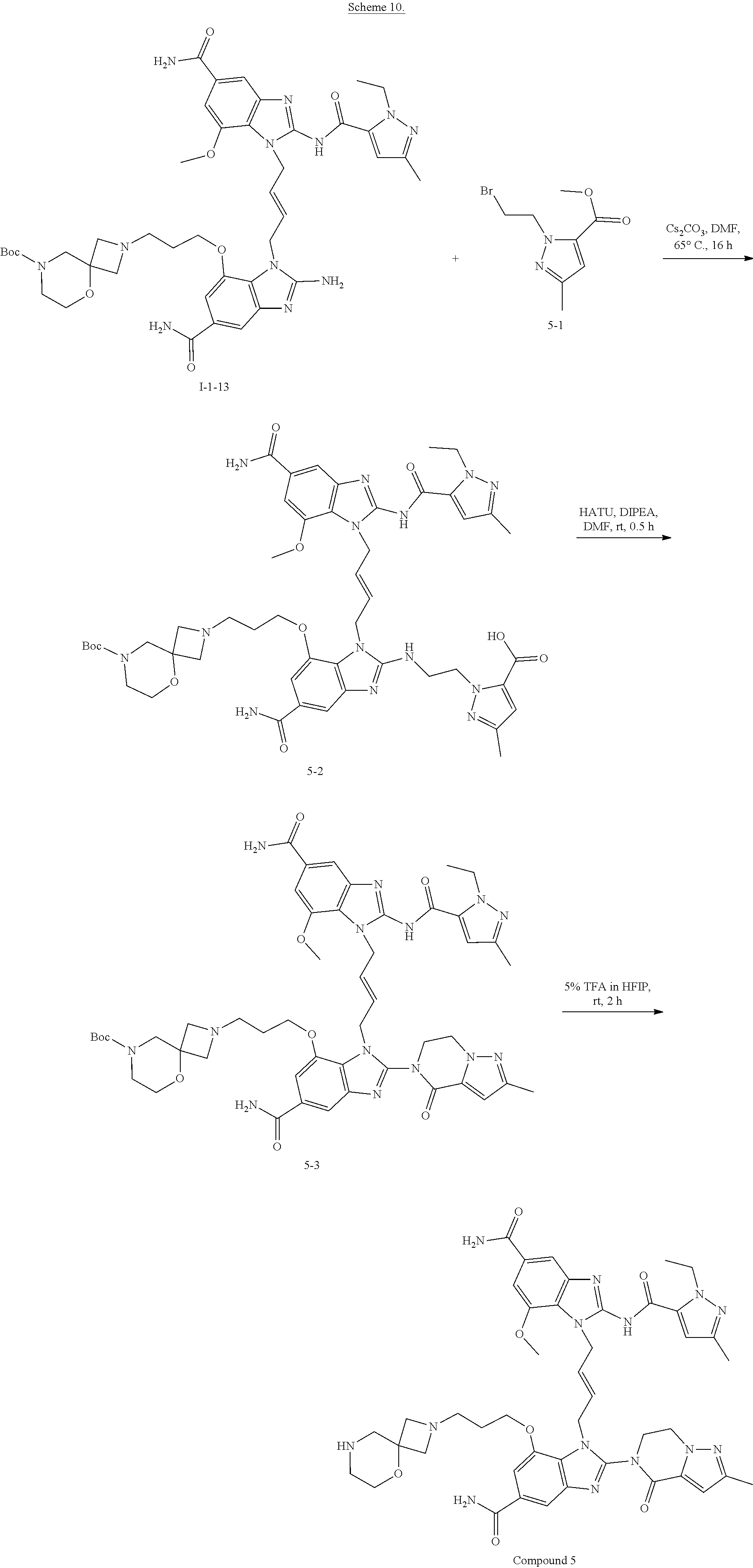

2-[2-[[7-[3-tert-Butoxycarbonyl-5-oxa-2,8-diazaspiro[3.5]nonan-2-yl)propoxy]-5-carbamoyl-1-[(E)-4-[5-carbamoyl-2-[(2-ethyl-5-methyl-pyrazole-3-carbonyl)amino]-7-methoxy-benzimidazol-1-yl]but-2-enyl]benzimidazol-2-yl]amino]ethyl]-5-methyl-pyrazole-3-carboxylic acid (5-2): A stirred solution of tert-butyl 2-[3-[2-amino-6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]benzimidazol-4-yl]oxypropyl]-5-oxa-2,8-diazaspiro[3.5]nonane-8-carboxylate (I-1-13) (50 mg, 0.06 mmol), cesium carbonate (134 mg, 0.41 mmol) and methyl 2-(2-bromoethyl)-5-methyl-pyrazole-3-carboxylate (5-1) (44 mg, 0.18 mmol) in DMF (4 mL) was heated to 65° C. for 16 hours. LC-MS showed major desired product formation. The reaction mixture was cooled to room temperature before it was filtered through a plug. Filtrate was purified by reverse phase preparative-HPLC (Column: Phenomenex kinetex 5 μm C₁₈ 100 Å, 250×21.2 mm and using 5-40% ACN: 0.1% TFA in water as eluent, Flow rate: 21.2 mL/min, run time: 25 mins to afford 2-[2-[[7-[3-(8-tert-butoxycarbonyl-5-oxa-2,8-diazaspiro[3.5]nonan-2-yl)propoxy]-5-carbamoyl-1-[(E)-4-[5-carbamoyl-2-[(2-ethyl-5-methyl-pyrazole-3-carbonyl)amino]-7-methoxy-benzimidazol-1-yl]but-2-enyl]benzimidazol-2-yl]amino]ethyl]-5-methyl-pyrazole-3-carboxylic acid (5-2) (23 mg, 39% yield) as a white solid. ¹H NMR (400 MHz, D₂O) δ 7.34 (d, J=1.3 Hz, 1H), 7.26 (s, 1H), 7.20 (s, 1H), 7.06 (s, 1H), 6.30 (s, 1H), 6.22 (s, 1H), 5.55 (s, 2H), 4.86 (s, 2H), 4.54 (s, 2H), 4.20 (q, J=7.2 Hz, 2H), 4.05 (s, 2H), 3.93 (s, 2H), 3.75 (s, 3H), 3.68 (d, J=11.3 Hz, 2H), 3.54 (s, 3H), 3.46 (s, 2H), 3.30 (s, 2H), 3.23 (d, J=8.1 Hz, 2H), 2.91 (d, J=0.6 Hz, 1H), 2.76 (d, J=0.9 Hz, 1H), 2.10 (s, 3H), 1.79 (s, 2H), 1.73 (s, 3H), 1.34 (d, J=15.2 Hz, 9H), 1.11 (t, J=7.1 Hz, 3H). LC-MS [ESI]: Calculated for C₄₉H₆₂N₁₄O₁₀ [M+H⁺]: 1007.50, Found: 1007.60.

tert-Butyl 2-[3-[6carbamoyl-3-[(E)4-[5-carbamoyl-2-[(2-ethyl-5-methyl-pyrazole-3-carbonyl)amino]-7-methoxy-benzimidazol-1-yl]but-2-enyl]-2-(2-methyl-4-oxo-6,7-dihydropyrazolo[1,5-a]pyrazin-5-yl)benzimidazol-4-yl]oxypropyl]-5-oxa-2,8-diazaspiro[3.5]nonane-8-carboxylate (5-3): To a stirred solution of 2-[2-[[7-[3-(8-tert-butoxycarbonyl-5-oxa-2,8-diazaspiro[3.5]nonan-2-yl)propoxy]-5-carbamoyl-1-[(E)-4-[5-carbamoyl-2-[(2-ethyl-5-methyl-pyrazole-3-carbonyl)amino]-7-methoxy-benzimidazol-1-yl]but-2-enyl]benzimidazol-2-yl]amino]ethyl]-5-methyl-pyrazole-3-carboxylic acid (5-2, 21 mg, 0.02 mmol) in DMF (2 mL) at room temperature was added DIPEA (0.02 mL, 0.1 mmol) followed by the addition of HATU (11.89 mg, 0.03 mmol). The solution was stirred at room temperature for 0.5 hour. Major desired product formation was observed by LC-MS. Reaction mixture was mixed with the reaction mixture previously obtained from other reactions and purified by prep HPLC (Column: Phenomenex kinetex 5 μm C₁₈ 100 Å, 250×21.2 mm and using 5-50% ACN: 0.1% TFA in water as eluent, Flow rate: 21.2 mL/min, run time: 30 mins to afford tert-butyl 2-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-(2-methyl-4-oxo-6,7-dihydropyrazolo[1,5-a]pyrazin-5-yl)benzimidazol-4-yl]oxypropyl]-5-oxa-2,8-diazaspiro[3.5]nonane-8-carboxylate (5-3) (12 mg, 55% yield) as a white solid. LC-MS [ESI]: Calculated for C₄₉H₆₀N₁₄O₉ [M+H⁺]: 989.50, Found: 989.60.

1-[(E)-4-[5-Carbamoyl-2-(2-methyl-4-oxo-6,7-dihydropyrazolo[1,5-a]pyrazin-5-yl)-7-[3-(5-oxa-2,8-diazaspiro[3.5]nonan-2-yl)propoxy]benzimidazol-1-yl]but-2-enyl]-2-[(2-ethyl-5-methyl-pyrazole-3-carbonyl)amino]-7-methoxy-benzimidazole-5-carboxamide (Compound 5): To a stirred solution of tert-butyl 2-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-(2-methyl-4-oxo-6,7-dihydropyrazolo[1,5-a]pyrazin-5-yl)benzimidazol-4-yl]oxypropyl]-5-oxa-2,8-diazaspiro[3.5]nonane-8-carboxylate (5-3, 11 mg, 0.01 mmol) in HFIP (0.5 mL) at room temperature was added 5% TFA in HFIP (1.7 mL, 1.11 mmol). The solution was stirred at room temperature for 2 hours. Reaction mixture was concentrated under reduced pressure before it was taken in DMF and purified by reverse phase preparative HPLC (Column: Phenomenex kinetex 5 μm C₁₈ 100 Å, 250×21.2 mm and using 5-40% ACN: 0.1% TFA in water as eluent, Flow rate: 21.2 mL/min, run time: 25 mins to afford 1-[(E)-4-[5-carbamoyl-2-(2-methyl-4-oxo-6,7-dihydropyrazolo[1,5-a]pyrazin-5-yl)-7-[3-(5-oxa-2,8-diazaspiro[3.5]nonan-2-yl)propoxy]benzimidazol-1-yl]but-2-enyl]-2-[(2-ethyl-5-methyl-pyrazole-3-carbonyl)amino]-7-methoxy-benzimidazole-5-carboxamide (Compound 5, 3.1 mg, 0.004 mmol, 32% yield) as a white solid (di TFA salt). ¹H NMR (400 MHz, DMSO) δ 10.55 (d, J=98.2 Hz, 2H), 9.22 (s, 2H), 8.00 (s, 2H), 7.67 (s, 2H), 7.45 (s, 1H), 7.38 (s, 2H), 7.26 (s, 1H), 6.32 (s, 2H), 5.64 (d, J=14.9 Hz, 2H), 4.95 (s, 3H), 4.56 (dt, J=13.2, 7.2 Hz, 5H), 4.41-4.29 (m, 2H), 4.28-4.07 (m, 5H), 3.88 (d, J=22.6 Hz, 4H), 3.15 (s, 3H), 2.13 (s, 5H), 1.94 (s, 3H), 1.24 (s, 3H). LC-MS [ESI]: Calculated for C₄₄H₅₂N₁₄O₇ [M+H⁺]: 889.42, Found: 889.40.

### Synthesis 2F. Compound 6, Compound 7, Compound 8, Compound 9, Compound 10, Compound 11, Compound 12, Compound 13, Compound 14, and Compound 15

Compound 6, Compound 7, Compound 8, Compound 9, Compound 10, Compound 11, Compound 12, Compound 13, Compound 14, and Compound 15 were synthesized as shown below via general Scheme 11.

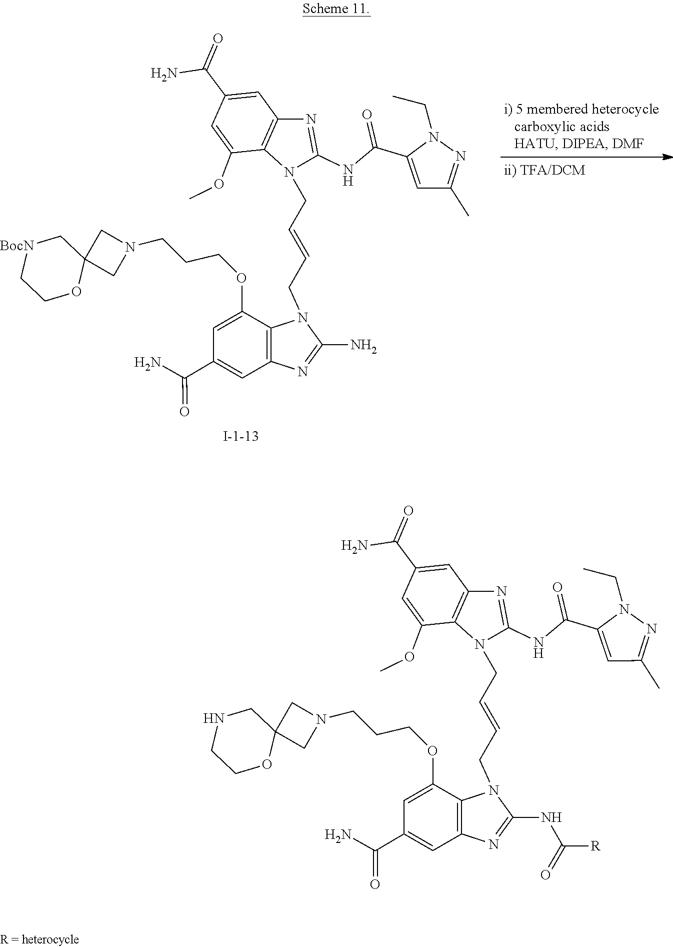

Amide coupling of compound I-1-13 with corresponding heterocycle carboxylic acids: To a mixture of tert-butyl (E)-2-(3-((2-amino-5-carbamoyl-1-(4-(5-carbamoyl-2-(1-ethyl-3-methyl-iH-pyrazole-5-carboxamido)-7-methoxy-1H-benzo[d]imidazol-1-yl)but-2-en-1-yl)-1H-benzo[d]imidazol-7-yl)oxy)propyl)-5-oxa-2,8-diazaspiro[3.5]nonane-8-carboxylate (I-1-13) (1 eq) and the corresponding five membered heterocycle carboxylic acid in DMF, was added DIEA (3 eq) and HATU (1.2 eq). The mixture was stirred at 50° C. and a clear solution formed. The reaction was stirred for 5 hours to drive the reaction to completion. The reaction solution was purified by reverse phase preparative HPLC (acetonitrile/water+0.1% formic acid) to afford Boc-protected Compound 6, Compound 7, Compound 8, Compound 9, Compound 10, Compound 11, Compound 12, Compound 13, Compound 14, or Compound 15.

Boc removal: A mixture of the Boc-protected intermediate (16.4 mg) in 2 mL of 5% (v/v) trifluoracetic acid in 1,1,1,3,3,3-hexafluoroisopropanol (HFIP) was stirred at room temperature for 0.5 hour. The reaction mixture was concentrated. The residue was diluted with DMSO (2 mL) and purified by reverse phase preparative HPLC (acetonitrile/water+0.1% TFA) to afford the final compound as its TFA salt. The final compounds were characterized by LCMS, ¹HNMR.

| Compound | Structure |
|---|---|
| No. | LCMS, ¹HNMR |
| 6 | 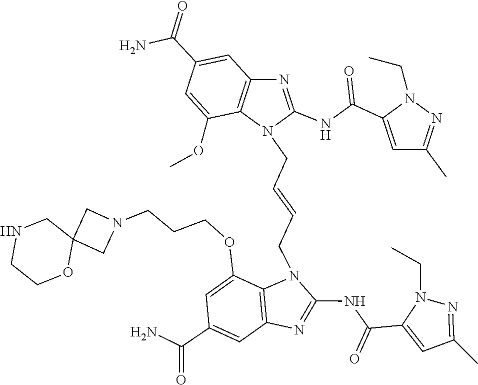 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(CNCCO4)C3)c21] ¹H NMR (400 MHz, DMSO) δ 8.99 (s, 2H), 7.97 (s, 2H), 7.64 (s, 2H), 7.38 (s, 1H), 7.31 (s, 2H), 6.52 (s, 2H), 5.79 (s, 2H), 4.91 (d, J = 14.4 Hz, 5H), 4.53 (s, 4H), 4.20-3.67 (m, 13H), 3.44 (s, 6H), 3.12 (s, 9H), 2.12 (d, J = 1.6 Hz, 6H), 1.78 (s, 3H), 1.27 (t, J = 7.1 Hz, 6H). LC- MS [ESI]: Calculated for C₄₄H₅₅N₁₄O₇ [M + H⁺]: 891.43, Found: 891.5. |
| 7 | 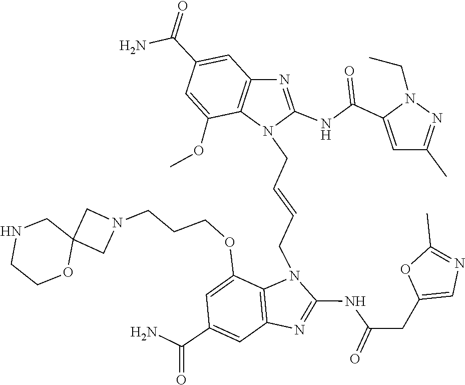 [SMILES: CCn1nc(C)cc1C(=O)Nc1nc2cc(C(N)=O)cc(OC)c2n1C/C=C/Cn1c(NC(=O)Cc2cnc(C)o2)nc2cc(C(N)=O)cc(OCCCN3CC4(CNCCO4)C3)c21] ¹H NMR (400 MHz, DMSO) δ 7.95 (d, J = 9.4 Hz, 2H), 7.66 (d, J = 1.4 Hz, 2H), 7.34 (s, 1H), 7.28 (d, J = 9.6 Hz, 2H), 7.22 (s, 1H), 6.79 (s, 1H), 6.52 (s, 1H), 5.83-5.73 (m, 1H), 5.67 (d, J = 15.9 Hz, 1H), 4.87 (d, J = 5.6 Hz, 2H), 4.80 (s, 2H), 4.53 (q, J = 7.0 Hz, 2H), 3.87 (d, J = 6.4 Hz, 2H), 3.72 (s, 2H), 3.69 (s, 3H), 3.14 (d, J = 7.3 Hz, 2H), 2.67 (s, 2H), 2.54 (s, 3H), 2.54 (d, J = 15.2 Hz, 1H), 2.36 (t, J = 6.8 Hz, 2H), 2.32 (s, 4H), 2.24 (s, 6H), 2.12 (s, 3H), 1.89 (s, 2H), 1.48 (d, J = 6.9 Hz, 2H), 1.28 (t, J = 7.1 Hz, 3H). LC-MS [ESI]: Calculated for C₄₃H₅₂N₁₃O₈ [M + H⁺]: 878.40, Found: 878.4. |
| 8 | 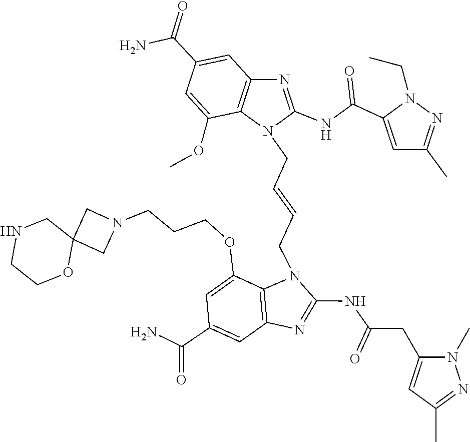 [SMILES: CCn1nc(C)cc1C(=O)Nc1nc2cc(C(N)=O)cc(OC)c2n1C/C=C/Cn1c(NC(=O)Cc2cc(C)nn2C)nc2cc(C(N)=O)cc(OCCCN3CC4(CNCCO4)C3)c21] ¹H NMR (400 MHz, DMSO) δ 7.95 (d, J = 12.4 Hz, 2H), 7.66 (d, J = 1.5 Hz, 2H), 7.36-7.20 (m, 4H), 6.52 (s, 1H), 5.86-5.72 (m, 2H), 5.64 (dd, J = 13.8, 7.6 Hz, 1H), 4.86 (d, J = 5.6 Hz, 2H), 4.79 (d, J = 5.4 Hz, 2H), 4.53 (q, J = 7.1 Hz, 2H), 3.89 (t, J = 6.2 Hz, 2H), 3.68 (s, 3H), 3.62 (s, 3H), 3.18-3.12 (m, 2H), 2.67 (s, 2H), 2.59-2.52 (m, 2H), 2.55 (s, 3H), 2.37 (t, J = 6.9 Hz, 2H), 2.12 (s, 3H), 2.05 (s, 3H), 1.88 (s, 1H), 1.54-1.46 (m, 2H), 1.27 (t, J = 7.1 Hz, 3H). LC-MS [ESI]: Calculated for C₄₄H₅₅N₁₄O₇ [M + H⁺]: 891.43, Found: 891.6. |
| 9 | 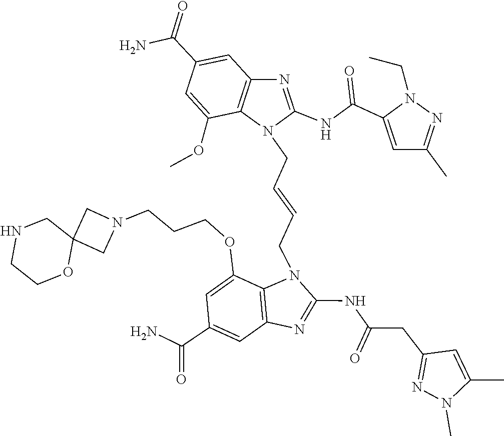 [SMILES: CCn1nc(C)cc1C(=O)Nc1nc2cc(C(N)=O)cc(OC)c2n1C/C=C/Cn1c(NC(=O)Cc2cc(C)n(C)n2)nc2cc(C(N)=O)cc(OCCCN3CC4(CNCCO4)C3)c21] ¹H NMR (400 MHz, DMSO) δ 7.95 (d, J = 7.2 Hz, 2H), 7.72 (s, 1H), 7.65 (d, J = 1.3 Hz, 1H), 7.34 (s, 1H), 7.26 (t, J = 3.7 Hz, 2H), 7.20 (d, J = 1.4 Hz, 1H), 6.53 (s, 1H), 5.89 (s, 1H), 5.85-5.73 (m, 1H), 5.68-5.60 (m, 1H), 4.82 (dd, J = 21.0, 5.7 Hz, 4H), 4.53 (q, J = 7.1 Hz, 2H), 3.87 (d, J = 6.3 Hz, 2H), 3.66-3.57 (m, 6H), 3.51 (d, J = 9.9 Hz, 2H), 3.43 (s, 1H), 3.32 (s, 2H), 3.18-3.08 (m, 3H), 2.67 (s, 2H), 2.62-2.50 (m, 4H), 2.37 (t, J = 6.9 Hz, 2H), 2.23-2.10 (m, 8H), 1.87 (s, 4H), 1.86 (s, 1H), 1.54-1.46 (m, 2H), 1.28 (t, J = 7.1 Hz, 3H), 1.16-1.03 (m, 2H), 1.02 (dd, J = 12.2, 6.8 Hz, 3H). LC-MS [ESI]: Calculated for C₄₄H₅₅N₁₄O₇ [M + H⁺]: 891.43, Found: 891.6. |
| 10 | 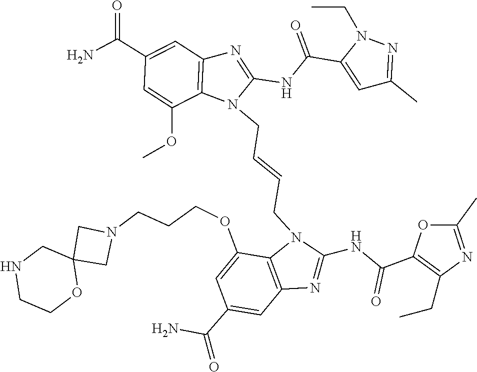 [SMILES: CCc1nc(C)oc1C(=O)Nc1nc2cc(C(N)=O)cc(OCCCN3CC4(CNCCO4)C3)c2n1C/C=C/Cn1c(NC(=O)c2cc(C)nn2CC)nc2cc(C(N)=O)cc(OC)c21] 1H NMR (400 MHz, DMSO) δ 7.96 (s, 2H), 7.67-7.61 (m, 2H), 7.38- 7.25 (m, 4H), 6.51 (d, J = 15.3 Hz, 2H), 5.81 (dt, J = 10.9, 5.1 Hz, 2H), 4.90 (s, 4H), 4.51 (q, J = 7.1 Hz, 2H), 3.95 (t, J = 6.0 Hz, 2H), 3.73 (s, 3H), 3.42 (s, 2H), 3.23 (d, J = 7.1 Hz, 2H), 2.85 (dd, J = 15.7, 8.2 Hz, 4H), 2.66 (s, 3H), 2.40 (s, 3H), 2.10 (s, 3H), 1.91 (d, J = 0.8 Hz, 3H), 1.59-1.51 (m, 2H), 1.26 (q, J = 6.7 Hz, 3H), 1.05 (t, J = 7.5 Hz, 3H). LC-MS [ESI]: Calculated for C₄₄H₅₄N₁₃O₈ [M + H⁺]: 892.41, Found: 892.5. |
| 11 | 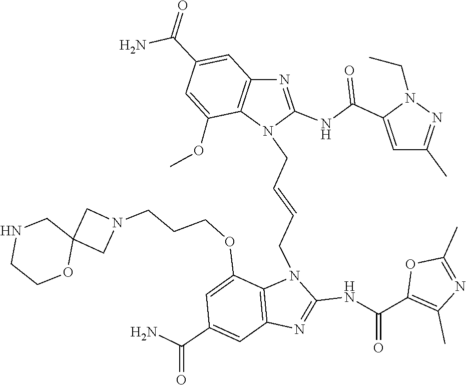 [SMILES: CCn1nc(C)cc1C(=O)Nc1nc2cc(C(N)=O)cc(OC)c2n1C/C=C/Cn1c(NC(=O)c2oc(C)nc2C)nc2cc(C(N)=O)cc(OCCCN3CC4(CNCCO4)C3)c21] ¹H NMR (400 MHz, DMSO) δ 12.83 (s, 1H), 12.77 (s, 1H), 10.49 (s, 1H), 9.08 (s, 2H), 7.97 (d, J = 9.9 Hz, 2H), 7.68-7.61 (m, 2H), 7.39 (s, 2H), 7.30 (s, 2H), 6.51 (s, 1H), 5.78 (s, 2H), 5.72 (d, J = 15.6 Hz, 1H), 4.90 (dd, J = 11.9, 4.8 Hz, 4H), 4.51 (t, J = 7.2 Hz, 2H), 4.34 (s, 1H), 4.12 (s, 2H), 4.04 (s, 3H), 3.95 (s, 1H), 3.87 (s, 1H), 3.79-3.69 (m, 4H), 3.11 (s, 2H), 2.39 (s, 3H), 2.31 (s, 3H), 2.09 (d, J = 16.5 Hz, 3H), 1.85 (s, 1H), 1.78 (s, 1H), 1.26 (q, J = 7.2 Hz, 3H). LC-MS [ESI]: Calculated for C₄₃H₅₂N₁₃O₈ [M + H⁺]: 878.40, Found: 878.4. |
| 12 | 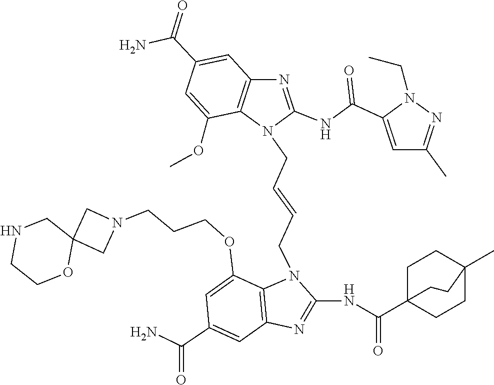 [SMILES: CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(CNCCO4)C3)c21] ¹H NMR (400 MHz, DMSO) δ 7.96 (s, 2H), 7.67 (s, 1H), 7.34 (d, J = 8.1 Hz, 2H), 7.25 (s, 2H), 6.54 (s, 1H), 5.80 (s, 2H), 4.86 (s, 2H), 4.75 (s, 1H), 4.52 (s, 2H), 3.99 (s, 2H), 3.73 (s, 3H), 3.38 (s, 1H), 3.22 (d, J = 7.2 Hz, 2H), 2.73 (s, 2H), 2.63 (d, J = 7.2 Hz, 2H), 2.59 (s, 4H), 2.13 (s, 3H), 1.91 (s, 2H), 1.61 (s, 9H), 1.24 (d, J = 9.5 Hz, 10H), 0.74 (s, 3H). LC-MS [ESI]: Calculated for C₄₇H₆₁N₁₂O₇ [M + H⁺]: 905.47, Found: 905.5. |
| 13 | 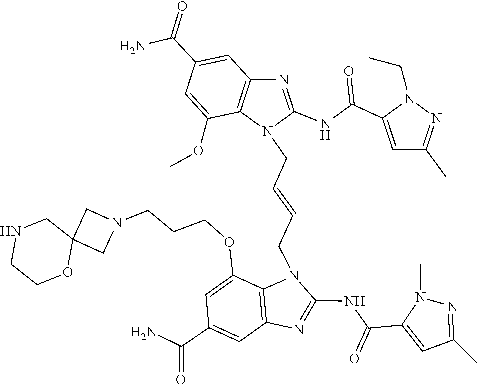 [SMILES: CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCN3CC4(CNCCO4)C3)c21] ¹H NMR (400 MHz, DMSO) δ 12.84 (s, 2H), 10.56 (s, 1H), 10.21 (s, 1H), 9.18 (s, 2H), 7.98 (s, 2H), 7.68-7.61 (m, 2H), 7.38 (s, 2H), 7.33-7.28 (m, 2H), 6.51 (d, J = 7.4 Hz, 2H), 5.85-5.69 (m, 2H), 4.91 (dd, J = 16.7, 4.8 Hz, 4H), 4.52 (q, J = 7.1 Hz, 2H), 4.35 (s, 1H), 4.12 (s, 1H), 4.03 (s, 3H), 3.95 (s, 1H), 3.88 (s, 1H), 3.77 (s, 1H), 3.70 (s, 3H), 3.34 (s, 3H), 3.28 (s, 3H), 3.11 (s, 2H), 2.10 (d, J = 6.3 Hz, 6H), 1.84 (s, 1H), 1.78 (s, 1H), 1.27 (t, J = 7.1 Hz, 3H). LC-MS [ESI]: Calculated for C₄₃H₅₃N₁₄O₇ [M + H⁺]: 877.41, Found: 877.4. |
| 14 | 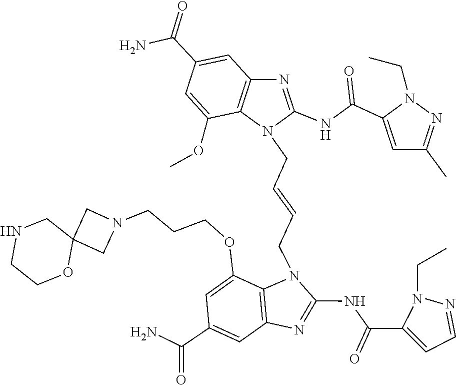 [SMILES: CCn1nc(C)cc1C(=O)Nc1nc2cc(C(N)=O)cc(OC)c2n1C/C=C/Cn1c(NC(=O)c2ccnn2CC)nc2cc(C(N)=O)cc(OCCCN3CC4(CNCCO4)C3)c21] ¹H NMR (400 MHz, DMSO) δ 12.85 (s, 2H), 10.41 (d, J = 133.8 Hz, 1H), 9.19 (s, 2H), 7.99 (d, J = 8.3 Hz, 2H), 7.69-7.62 (m, 2H), 7.42-7.30 (m, 5H), 6.71 (d, J = 2.0 Hz, 1H), 6.52 (s, 1H), 5.78 (dd, J = 9.8, 5.0 Hz, 2H), 4.92 (dd, J = 16.5, 4.3 Hz, 4H), 4.65-4.47 (m, 4H), 4.36 (s, 1H), 4.13 (s, 1H), 4.06 (s, 3H), 3.96 (s, 1H), 3.89 (s, 1H), 3.78 (s, 1H), 3.75-3.70 (m, 3H), 3.35 (s, 2H), 3.29 (s, 2H), 3.11 (s, 2H), 2.12 (s, 3H), 1.85 (s, 1H), 1.79 (s, 1H), 1.34-1.21 (m, 6H). LC-MS [ESI]: Calculated for C₄₃H₅₃N₁₄O₇ [M + H⁺]: 877.41, Found: 877.4. |
| 15 | 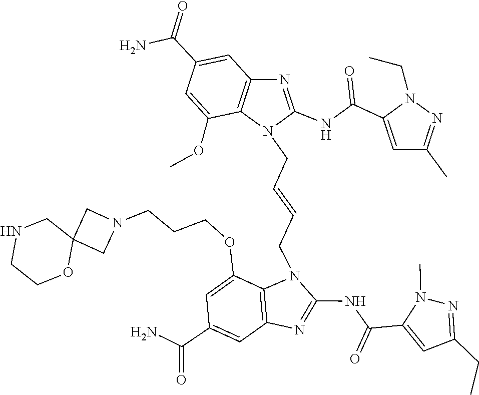 [SMILES: CCc1cc(C(=O)Nc2nc3cc(C(N)=O)cc(OCCCN4CC5(CNCCO5)C4)c3n2C/C=C/Cn2c(NC(=O)c3cc(C)nn3CC)nc3cc(C(N)=O)cc(OC)c32)n(C)n1] ¹H NMR (400 MHz, DMSO) δ 12.84 (s, 2H), 10.60 (s, 1H), 9.32 (s, 2H), 7.97 (s, 1H), 7.65 (d, J = 7.8 Hz, 2H), 7.38 (s, 2H), 7.32 (d, J = 8.6 Hz, 2H), 6.53 (d, J = 10.4 Hz, 2H), 5.77 (d, J = 23.0 Hz, 2H), 4.94 (s, 2H), 4.89 (s, 2H), 4.52 (d, J = 7.5 Hz, 2H), 4.38 (s, 1H), 4.04 (s, 4H), 3.71 (d, J = 8.8 Hz, 4H), 3.38 (d, J = 22.4 Hz, 2H), 3.09 (s, 2H), 2.11 (s, 3H), 1.81 (d, J = 32.9 Hz, 2H), 1.26 (q, J = 6.1 Hz, 3H), 1.12 (t, J = 7.6 Hz, 3H). LC- MS [ESI]: Calculated for C₄₄H₅₅N₁₄O₇ [M + H⁺]: 891.43, Found: 891.6. |

### Synthesis 2G. Compound 16, Compound 17, Compound 18, Compound 19 and Compound 20

Compound 16, Compound 17, Compound 18, Compound 19, and Compound 20 were synthesized as shown below via general Scheme 12 (via Compound 6) or Scheme 13 (via Compound 10) by coupling the compound 6 or 10 to Boc-protected amino acid in the presence of HBTU and DIPEA and DMF Boc protected compounds were deprotected using TFA to afford the final compounds. The final compounds were purified by reverse phase preparative HPLC and characterized by LCMS, ¹HNMR.

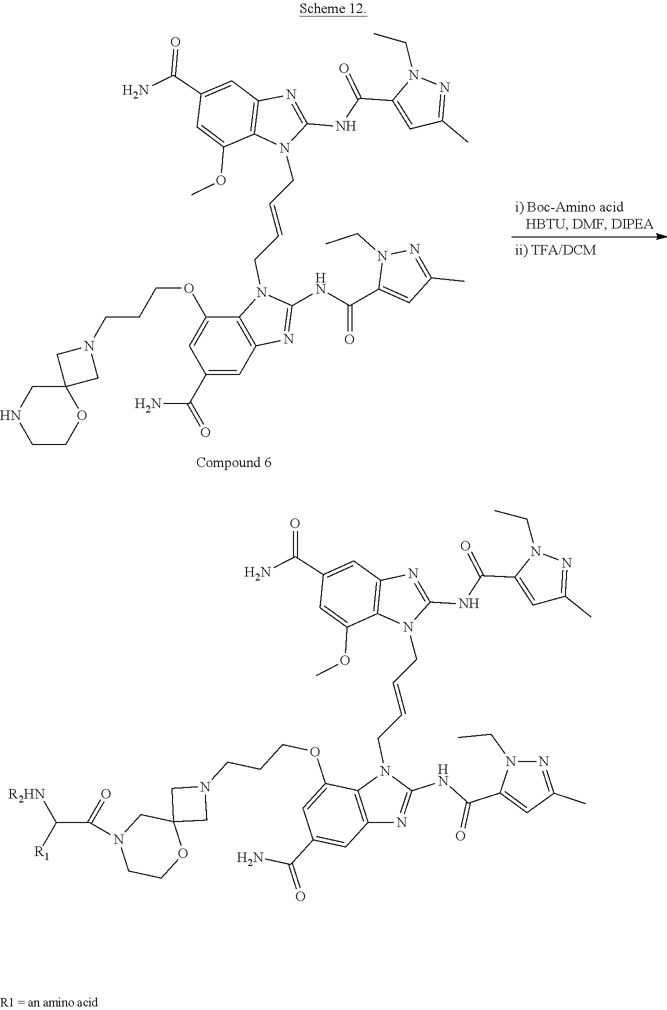

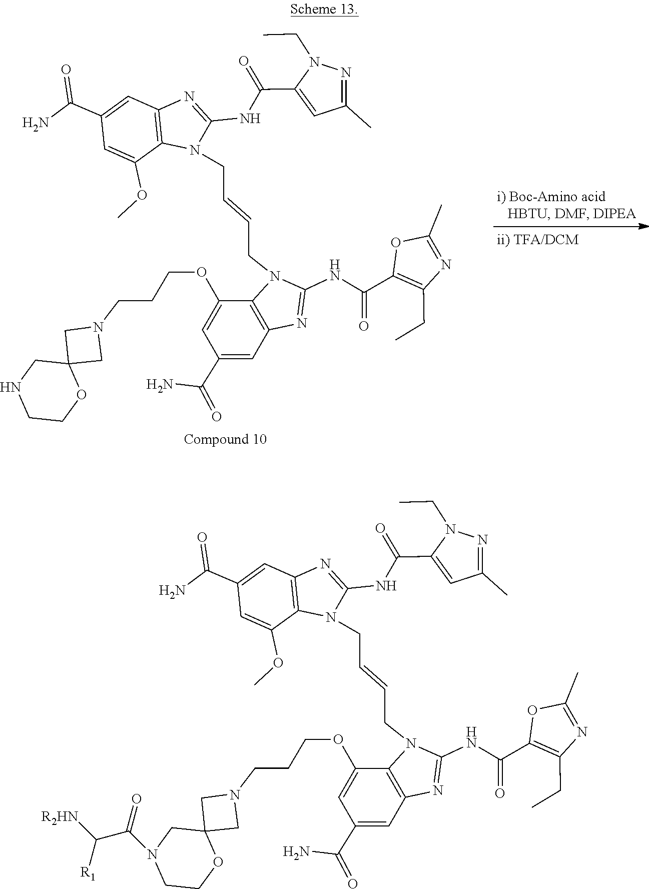

| Compound | Structure |
|---|---|
| No. | LCMS, ¹HNMR |
| 16 | 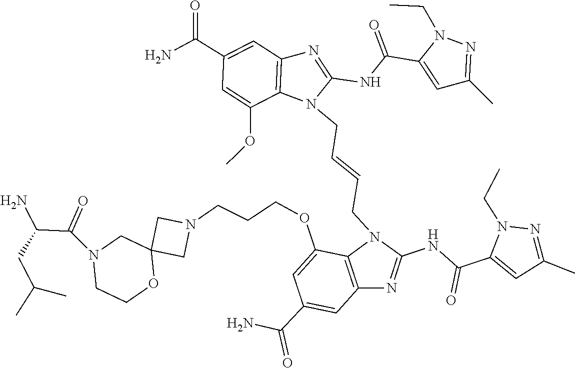 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(C3)CN(C(=O)[C@@H](N)CC(C)C)CCO4)c21] ¹H NMR (400 MHz, DMSO) δ 7.94 (s, 2H), 7.65 (d, J = 5.1 Hz, 2H), 7.45- 7.20 (m, 3H), 6.54 (d, J = 19.3 Hz, 2H), 5.83 (dt, J = 8.8, 4.9 Hz, 2H), 4.92 (d, J = 16.7 Hz, 3H), 4.54 (p, J = 7.0 Hz, 3H), 3.96 (s, 2H), 3.72 (s, 3H), 3.48 (d, J = 21.9 Hz, 4H), 3.11 (s, 1H), 2.62 (d, J = 7.3 Hz, 1H), 2.41 (s, 1H), 2.12 (d, J = 7.8 Hz, 4H), 1.83-1.66 (m, 1H), 1.54 (s, 2H), 1.35- 1.14 (m, 6H), 0.94-0.70 (m, 4H). LC-MS [ESI]: Calculated for C₅₀H₆₆N₁₅O₈ [M + H⁺]: 1004.51, Found: 1004.52. |
| 17 | 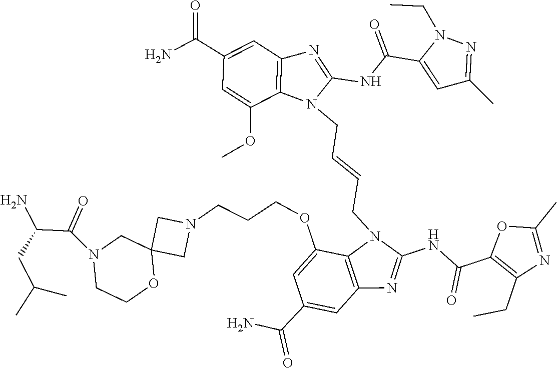 [SMILES: CCc1nc(C)oc1C(=O)Nc1nc2cc(C(N)=O)cc(OCCCN3CC4(C3)CN(C(=O)[C@@H](N)CC(C)C)CCO4)c2n1C/C=C/Cn1c(NC(=O)c2cc(C)nn2CC)nc2cc(C(N)=O)cc(OC)c21] ¹H NMR (400 MHz, DMSO) δ 12.83 (s, 1H), 12.77 (s, 1H), 10.12 (s, OH), 8.16 (s, 2H), 7.98 (d, J = 12.0 Hz, 2H), 7.65 (d, J = 9.9 Hz, 2H), 7.38 (s, 2H), 7.31 (d, J = 3.9 Hz, 2H), 6.50 (s, 1H), 5.79 (s, 1H), 5.73 (q, J = 7.8 Hz, 1H), 4.90 (t, J = 6.8 Hz, 4H), 4.51 (q, J = 7.1 Hz, 2H), 4.36 (s, 1H), 3.71 (d, J = 9.5 Hz, 3H), 3.61-3.49 (m, 1H), 2.82 (q, J = 7.5 Hz, 2H), 2.40 (s, 3H), 2.11 (s, 3H), 1.80 (s, 2H), 1.55 (s, 1H), 1.49-1.38 (m, 1H), 1.26 (t, J = 7.1 Hz, 3H), 1.03 (t, J = 7.5 Hz, 3H), 0.98-0.84 (m, 6H). LC-MS [ESI]: Calculated for C₅₀H₆₅N₁₄O₉ [M + H⁺]: 1005.50, Found: 1005.8. |
| 18 | 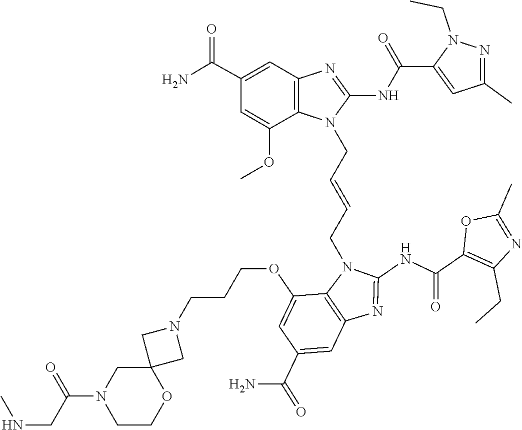 [SMILES: CCc1nc(C)oc1C(=O)Nc1nc2cc(C(N)=O)cc(OCCCN3CC4(C3)CN(C(=O)CNC)CCO4)c2n1C/C=C/Cn1c(NC(=O)c2cc(C)nn2CC)nc2cc(C(N)=O)cc(OC)c21] ¹H NMR (400 MHz, DMSO) δ 12.78 (s, 2H), 8.78 (s, 2H), 7.98 (d, J = 15.2 Hz, 2H), 7.65 (d, J = 10.5 Hz, 2H), 7.38 (s, 2H), 7.31 (d, J = 4.8 Hz, 2H), 6.49 (s, 1H), 5.78 (dd, J = 21.2, 15.9 Hz, 2H), 4.90 (t, J = 6.8 Hz, 4H), 4.51 (q, J = 7.2 Hz, 2H), 4.24 (s, 8H), 4.09-4.03 (m, 8H), 3.89 (s, 3H), 3.76- 3.68 (m, 5H), 3.59 (s, 2H), 3.46 (s, 1H), 3.31 (d, J = 20.4 Hz, 2H), 2.83 (q, J = 7.2 Hz, 2H), 2.59 (q, J = 4.9 Hz, 4H), 2.40 (d, J = 1.6 Hz, 3H), 2.10 (s, 3H), 1.81 (s, 3H), 1.26 (t, J = 7.1 Hz, 3H), 1.07-0.99 (m, 3H). LC- MS [ESI]: Calculated for C₄₇H₅₉N₁₄O₉ [M + H⁺]: 963.45, Found: 963.6. |
| 19 | 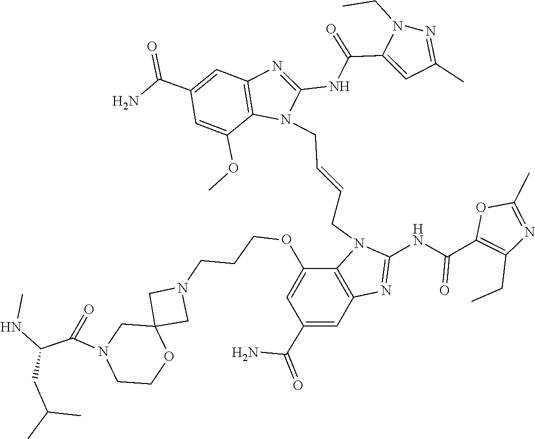 [SMILES: CCc1nc(C)oc1C(=O)Nc1nc2cc(C(N)=O)cc(OCCCN3CC4(C3)CN(C(=O)[C@H](CC(C)C)NC)CCO4)c2n1C/C=C/Cn1c(NC(=O)c2cc(C)nn2CC)nc2cc(C(N)=O)cc(OC)c21] ¹H NMR (400 MHz, DMSO) δ 12.77 (s, 2H), 8.96 (s, 2H), 7.97 (d, J = 9.1 Hz, 2H), 7.68-7.61 (m, 2H), 7.37 (s, 2H), 7.31 (d, J = 4.1 Hz, 2H), 6.49 (s, 1H), 5.82-5.66 (m, 1H), 4.90 (t, J = 7.3 Hz, 4H), 4.51 (q, J = 7.1 Hz, 2H), 4.44 (s, 1H), 4.04 (d, J = 9.9 Hz, 6H), 3.89 (s, 1H), 3.71 (d, J = 8.9 Hz, 3H), 3.61-3.40 (m, 2H), 2.81 (dd, J = 7.6, 3.7 Hz, 2H), 2.55 (d, J = 6.0 Hz, 1H), 2.40 (s, 3H), 2.10 (s, 3H), 1.83 (s, 2H), 1.58 (d, J = 7.5 Hz, 3H), 1.26 (t, J = 7.1 Hz, 3H), 1.08-0.98 (m, 3H), 0.96-0.82 (m, 6H). LC- MS [ESI]: Calculated for C₅₁H₆₇N₁₄O₉ [M + H⁺]: 1019.51, Found: 1019.6. |
| 20 | 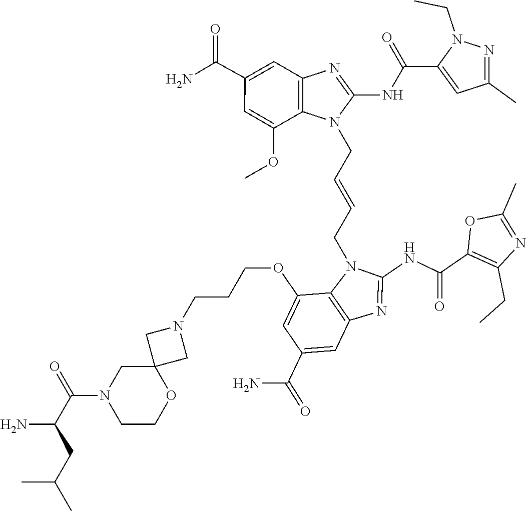 [SMILES: CCc1nc(C)oc1C(=O)Nc1nc2cc(C(N)=O)cc(OCCCN3CC4(C3)CN(C(=O)[C@H](N)CC(C)C)CCO4)c2n1C/C=C/Cn1c(NC(=O)c2cc(C)nn2CC)nc2cc(C(N)=O)cc(OC)c21] ¹H NMR (400 MHz, DMSO) δ 12.78 (s, 1H), 10.61 (s, 1 H), 10.26 (s, 1H), 8.19 (s, 3H), 7.98 (d, J = 11.6 Hz, 2H), 7.68-7.61 (m, 2H), 7.38 (s, 2H), 7.31 (d, J = 4.0 Hz, 2H), 6.49 (s, 1H), 5.85-5.67 (m, 1H), 4.89 (d, J = 8.0 Hz, 4H), 4.51 (q, J = 7.1 Hz, 2H), 4.36 (s, 1H), 4.19 (s, 1H), 3.93 (q, J = 13.3 Hz, 1H), 3.72 (d, J = 8.8 Hz, 3H), 3.66-3.56 (m, 1H), 3.48 (q, J = 13.9 Hz, 1H), 2.82 (q, J = 7.5 Hz, 2H), 2.40 (s, 3H), 2.10 (s, 3H), 1.99- 1.38 (m, 6H), 1.26 (t, J = 7.1 Hz, 3H), 1.03 (t, J = 7.5 Hz, 3H), 0.98-0.84 (m, 6H). LC-MS [ESI]: Calculated for C₅₀H₆₅N₁₄O₉ [M + H⁺]: 1005.50, Found: 1005.6 |

### Synthesis 211. Compound 21

Compound 21 was synthesized using the same methods as described herein (e.g., Scheme 8), as shown in Scheme 14 below, for the synthesis of Compound 1, but starting from Intermediate I-4.

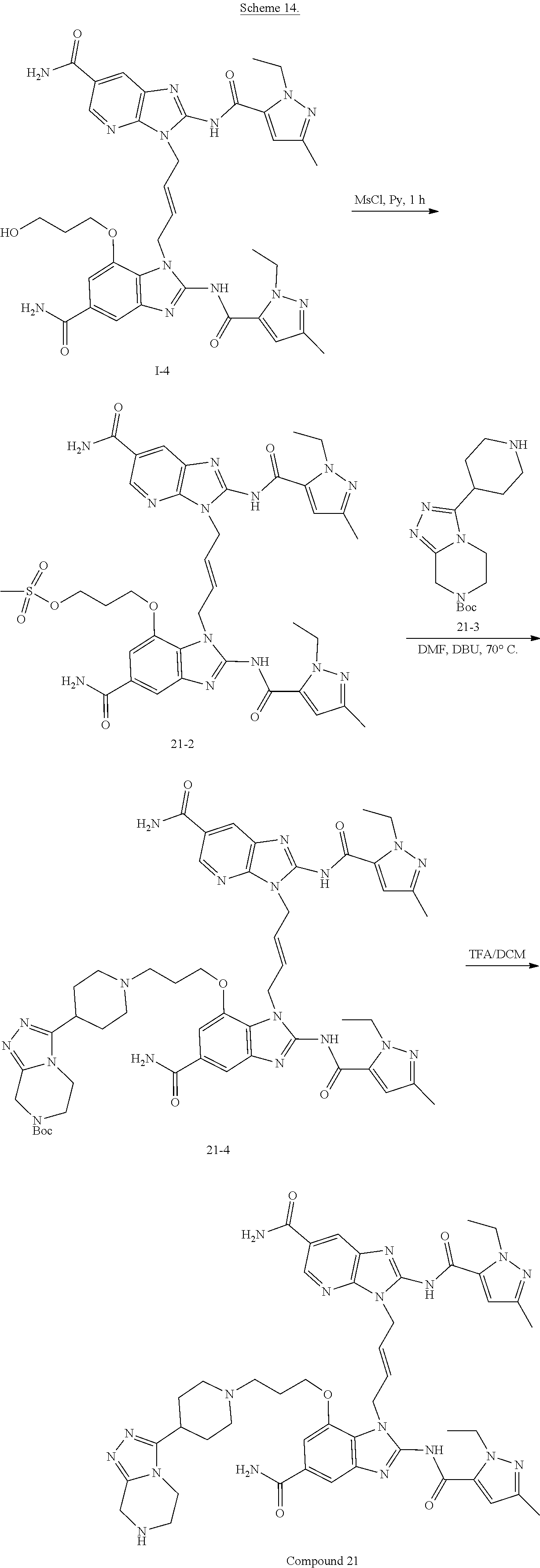

### Synthesis 2I. Compound 22

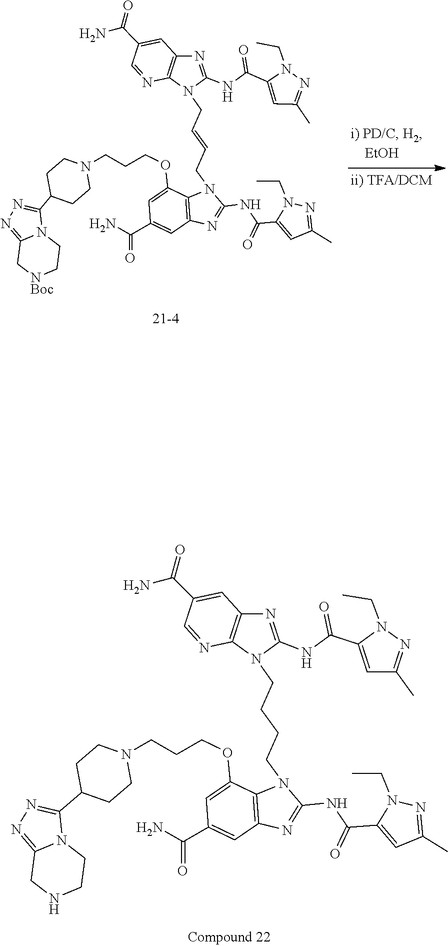 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)OC(C)(C)C)C5)CC3)c21 ; CCn1nc(C)cc1C(=O)Nc1nc2cc(C(N)=O)cnc2n1CCCCn1c(NC(=O)c2cc(C)nn2CC)nc2cc(C(N)=O)cc(OCCCN3CCC(c4nnc5n4CCNC5)CC3)c21]

Compound 22-1 was reduced with Pd/C, H₂ in EtOH and the compound was subsequently treated with TFA/DCM. The crude compound was purified by reverse phase preparative HPLC to give Compound 22 as a solid. LC-MS [ESI]: Calculated for C₄₆H₅₈N₁₈O₅[M+H⁺]: 943.48, Found: 943.6.

### Synthesis 2J. Compound 24

Compound 24 was synthesized as described in Scheme 15.

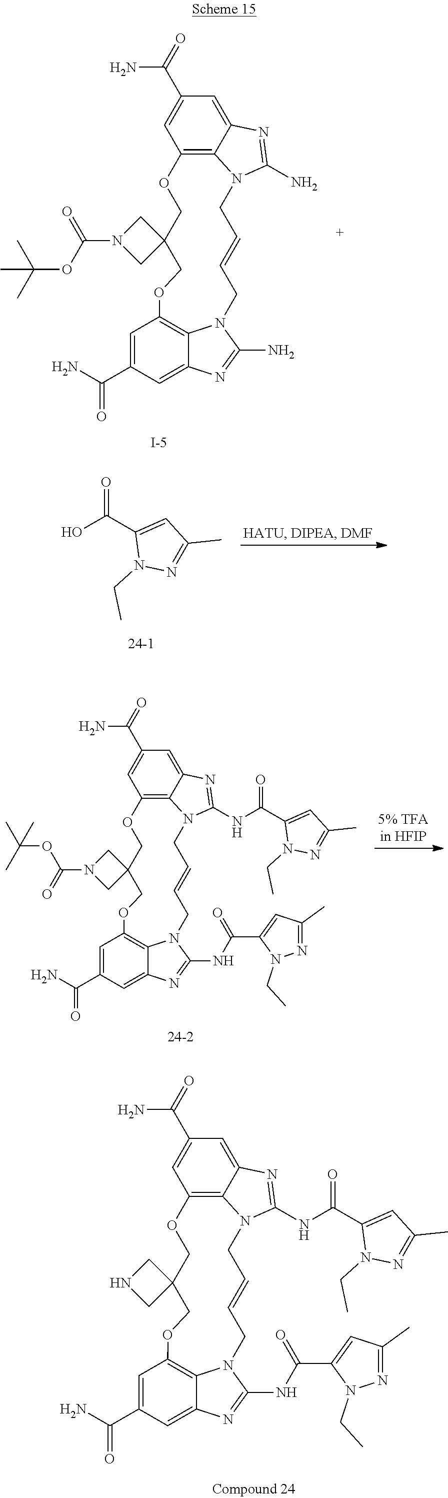

tert-Butyl (3E)-11,21-dicarbamoyl-7,25-bis[(2-ethyl-5-methyl-pyrazole-3-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (24-2): To a stirred solution of 2-ethyl-5-methyl-pyrazole-3-carboxylic acid (24-1, 19.47 mg, 0.13 mmol) in DMF (0.5 mL) at room temperature was added HATU (55 mg, 0.15 mmol). The solution was stirred for 15 minutes. In another vial, to a stirred solution of tert-butyl (3E)-7,25-diamino-11,21-dicarbamoyl-spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (I-5, 15 mg, 0.02 mmol) in DMF (0.5 mL) at room temperature was added DIPEA (0.03 mL, 0.24 mmol). To this at room temperature was added the above active ester solution and the reaction mixture was stirred at 50° C. for 3 hours. The reaction mixture was cooled to room temperature, cold water was added to the reaction mixture, solids were filtered, washed with water followed by hexanes, and dried under reduced pressure to afford tert-butyl (3E)-11,21-dicarbamoyl-7,25-bis[(2-ethyl-5-methyl-pyrazole-3-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (24-2, 9 mg, 42% yield) as an off white solid. Crude was used in the next step without further purification. LC-MS [ESI]: Calculated for C₄₄H₅₁N₁₃O₈[M+H⁺]: 890.40, Found: 890.40.

(3E)-7,25-bis[(2-ethyl-5-methyl-pyrazole-3-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-11,21-dicarboxamide; 2,2,2-trifluoroacetic acid (Compound 24): To a stirred solution of tert-butyl (3E)-11,21-dicarbamoyl-7,25-bis[(2-ethyl-5-methyl-pyrazole-3-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (24-2, 9 mg, 0.01 mmol) in HFIP (0.5 mL) at room temperature was added 5% trifluoroacetic acid (0.77 mL, 0.51 mmol) in HFIP. The resulting solution was stirred at room temperature for 2 h. The reaction mixture was concentrated under reduced pressure and purified by reverse phase preparative-HPLC (Column: Phenomenex kinetex 5 μm C₁₈ 100 Å, 250×21.2 mm and using 5-35% ACN: 1% TFA in water as eluent, Flow rate: 21.2 mL/min, run time: 25 mins to afford (3E)-7,25-bis[(2-ethyl-5-methyl-pyrazole-3-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-11,21-dicarboxamide; 2,2,2-trifluoroacetic acid (Compound 24) (6.9 mg, 67% yield). ¹H NMR (400 MHz, DMSO) δ 9.16 (s, 1H), 8.02 (s, 1H), 7.83 (s, 1H), 7.78-7.73 (m, 1H), 7.47 (s, 1H), 6.55 (s, 1H), 5.39 (s, 1H), 4.86 (s, 2H), 4.59 (s, 2H), 4.50 (q, J=7.1 Hz, 2H), 4.09 (t, J=6.1 Hz, 2H), 2.06 (s, 3H), 1.24 (t, J=7.1 Hz, 3H). LC-MS [ESI]: Calculated for C₃₉H₄₃N₁₃O₆[M+H⁺]: 790.40, Found: 790.40.

### Synthesis 2K. Compound 25

Compound 25 was synthesized as described in Scheme 16.

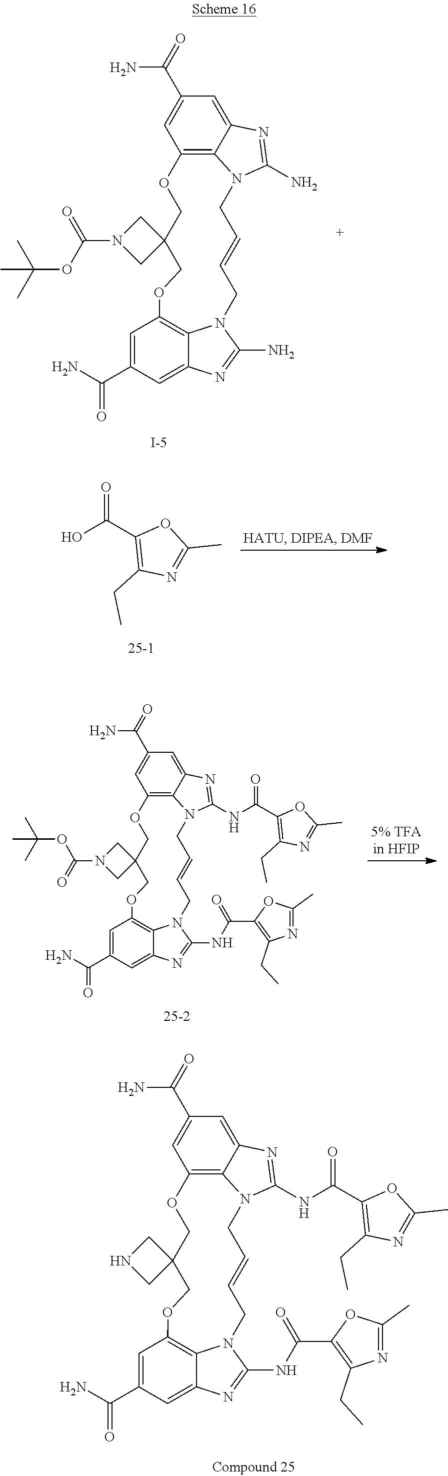

tert-butyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]1′-carboxylate (25-2): To a stirred solution of 4-ethyl-2-methyl-oxazole-5-carboxylic acid (25-1, 26.1 mg, 0.17 mmol) in DMF (1 mL) at room temperature was added HATU (74 mg, 0.19 mmol). The solution was stirred at room temperature for 15 minutes. In another vial, to a stirred solution of tert-butyl (3E)-7,25-diamino-11,21-dicarbamoyl-spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (I-5, 20 mg, 0.03 mmol) in DMF (1 mL) at room temperature was added DIPEA (0.05 mL, 0.32 mmol). To this at room temperature was added the above active ester solution and the reaction mixture was stirred at room temperature for 2 hours. The reaction mixture was cooled to room temperature, cold water was added to the reaction mixture, solids were filtered, washed with water followed by hexanes, and dried under reduced pressure to afford the crude. The crude material was purified by reverse phase preparative HPLC (Column: Phenomenex kinetex 5 μm C₁₈ 100 Å, 250×21.2 mm and using 5-70% ACN: 1% TFA in water as eluent, Flow rate: 21.2 mL/min, run time: 30 mins to afford tert-butyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (25-2, 11 mg, 38% yield) as a white solid. LC-MS [ESI]: Calculated for C₄₄H₄₉N₁₁O₁₀ [M+H⁺]: 892.40, Found: 892.40.

(3E)-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-11,21-dicarboxamide (Compound 25): To a stirred solution of tert-butyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (25-2, 10 mg, 0.01 mmol) in HFIP (0.5 mL) at room temperature was added 5% TFA in HFIP (0.86 mL, 0.56 mmol). The solution was stirred at room temperature for 2 hours. Reaction mixture was concentrated under reduced pressure and crude was purified by prep-HPLC (Column: Phenomenex kinetex 5 μm C₁₈ 100 Å, 250×21.2 mm and using 5-50% ACN: 1% TFA in water as eluent, Flow rate: 21.2 mL/min, run time: 30 mins to afford (3E)-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-11,21-dicarboxamide Compound 25 (7 mg, 78.8% yield) as a white solid (di TFA salt). ¹H NMR (400 MHz, DMSO) δ 12.85 (s, 2H), 9.02 (s, 2H), 7.99 (s, 2H), 7.79-7.75 (m, 2H), 7.73 (s, 2H), 7.46 (s, 2H), 5.38 (s, 2H), 4.83 (s, 4H), 4.55 (s, 4H), 4.08 (t, J=6.2 Hz, 4H), 2.79 (q, J=7.5 Hz, 4H), 2.37 (s, 6H), 0.98 (t, J=7.5 Hz, 6H). LC-MS [ESI]: Calculated for C₃₉H₄₁N₁₁O₈ [M+H⁺]: 792.32, Found: 792.40.

### Synthesis 2L. Compound 26

Compound 26 was synthesized as described in Scheme 17.

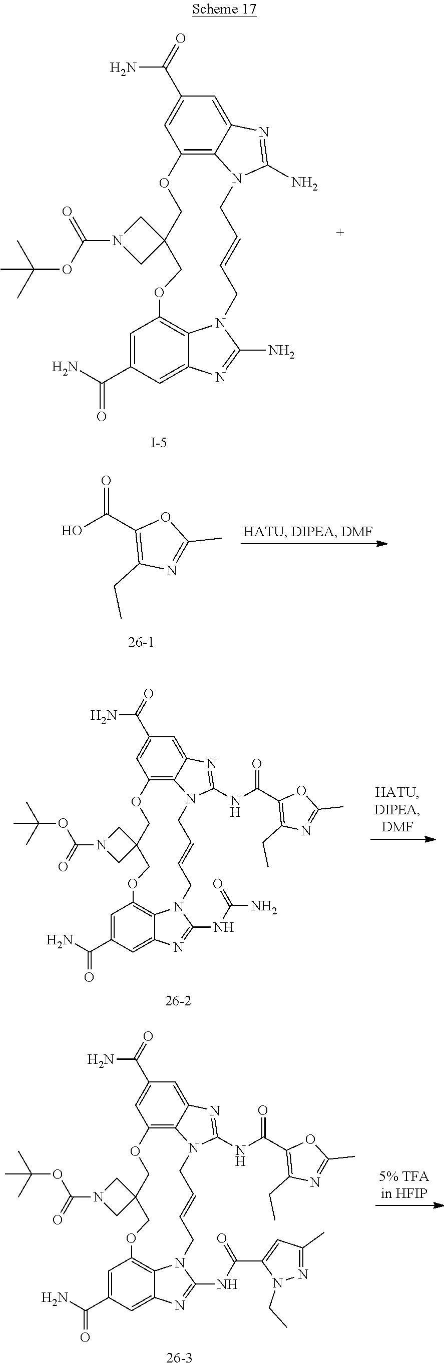

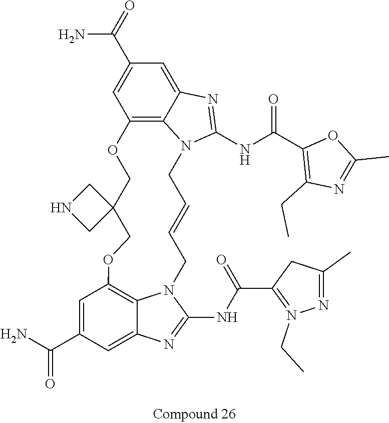

tert-Butyl (3E)-7-amino-11,21-dicarbamoyl-25-[(4-ethyl-2-methyl-oxazole-5-carbonyl)amino]spiro[14,18-dioxa-1,6,8,24-tetrazapentacyclo[17.6.1.16,9.023,26.013,27]heptacosa-3,9,11,13 (27), 19 (26), 20,22,24-nonaene-16,3′-azetidine]-1′-carboxylate (26-2): To a stirred solution of 4-ethyl-2-methyl-oxazole-5-carboxylic acid (26-1, 2.51 mg, 0.02 mmol) in DMF (0.500 mL) at room temperature was added HATU (7.39 mg, 0.02 mmol). The solution was stirred for 15 minutes. In another vial, to a stirred solution of tert-butyl (3E)-7,25-di amino-11,21-dicarbamoyl-spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (1-5, 20 mg, 0.03 mmol) in DMF (0.5000 mL) at room temperature was added DTPEA (0. mL, 0.03 mmol). To this at room temperature was added the above active ester solution and the reaction mixture was stirred at room temperature for 16 hours. The reaction mixture was purified by prep-HPLC (Column: Phenomenex kinetex 5 μm C₁₈ 100 Å, 250×21.2 mm and using 5-70% ACN: 1% TFA in water as eluent, Flow rate: 21.2 mL/min, run time: 30 mins to afford tert-butyl (3E)-7-amino-11,21-dicarbamoyl-25-[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (26-2, 10 mg, 41% yield) as a white solid. LC-MS [ESI]: Calculated for C₃₇H₄₂N₁₀O₈ [M+H⁺]: 755.32, Found: 755.40.

tert-Butyl (3E)-11,21-dicarbamoyl-7-[(4-ethyl-2-methyl-oxazole-5-carbonyl)amino]-25-[(2-ethyl-5-methyl-pyrazole-3-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (26-3): To a stirred solution of 2-ethyl-5-methyl-pyrazole-3-carboxylic acid (6.13 mg, 0.04 mmol) in DMF (0.5000 mL) at room temperature was added HATU (20 mg, 0.05 mmol). The solution was stirred at room temperature for 15 minutes. In another vial, to a stirred solution of tert-butyl (3E)-7-amino-11,21-dicarbamoyl-25-[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (26-2, 10 mg, 0.01 mmol) in DMF (0.5 mL) at room temperature was added DIPEA (0.01 mL, 0.05 mmol). To this at room temperature was added the above active ester solution and the reaction mixture was stirred at this temperature for 16 hours. Cold water was added to the reaction mixture, solids were filtered, washed with water followed by hexanes and dried under reduced pressure to afford tert-butyl (3E)-11,21-dicarbamoyl-7-[(4-ethyl-2-methyl-oxazole-5-carbonyl)amino]-25-[(2-ethyl-5-methyl-pyrazole-3-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (26-3, 7 mg, 59% yield) as an off white solid. Crude was used in the next step without further purification. LC-MS [ESI]: Calculated for C₄₄H₅₀N₁₂O₉[M+H⁺]: 891.40, Found: 891.40.

(3E)-7-[(4-ethyl-2-methyl-oxazole-5-carbonyl)amino]-25-[(2-ethyl-5-methyl-pyrazole-3-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-11,21-dicarboxamide (Compound 26): To a stirred solution of tert-butyl (3E)-11,21-dicarbamoyl-7-[(4-ethyl-2-methyl-oxazole-5-carbonyl)amino]-25-[(2-ethyl-5-methyl-pyrazole-3-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (26-3, 7 mg, 0.01 mmol) in HFIP (0.5 mL) at room temperature was added 5% TFA in HFIP (0.67 mL, 0.44 mmol). The solution was stirred at room temperature for 2 hours. The reaction mixture was concentrated under reduced pressure and crude was purified by prep-HPLC (Column: Phenomenex kinetex 5 μm C₁₈ 100 Å, 250×21.2 mm and using 5-50% ACN: 1% TFA in water as eluent, Flow rate: 21.2 mL/min, run time: 30 mins to afford (3E)-7-[(4-ethyl-2-methyl-oxazole-5-carbonyl)amino]-25-[(2-ethyl-5-methyl-pyrazole-3-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-11,21-dicarboxamide (Compound 26, 4.5 mg, 73% yield) as white solid (di TFA salt). ¹H NMR (400 MHz, DMSO) δ 12.95 (s, 2H), 9.00 (s, 2H), 7.99 (s, 2H), 7.78 (s, 2H), 7.73 (d, J=2.6 Hz, 2H), 7.46 (s, 2H), 6.54 (s, 1H), 5.40 (s, 2H), 4.85 (d, J=11.2 Hz, 4H), 4.53 (d, J=22.9 Hz, 6H), 4.08 (q, J=4.3 Hz, 4H), 2.80 (q, J=7.5 Hz, 2H), 2.37 (s, 4H), 2.07 (s, 3H), 1.25 (t, J=7.0 Hz, 3H), 0.99 (t, J=7.5 Hz, 3H). LC-MS [ESI]: Calculated for (C₃₉H₄₂N₁₂O₇ [M+H⁺]: 791.33, Found: 791.40.

### Synthesis 2M. Compound 60

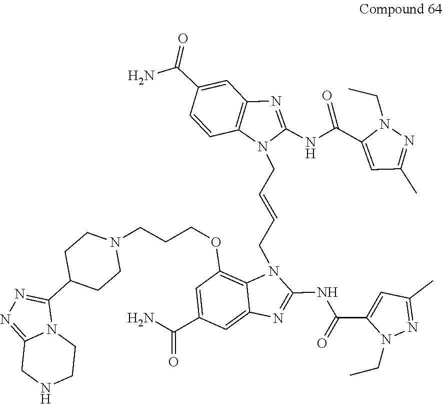 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCNC5)CC3)c21]

tert-butyl 3-[1-[3-[6-carbamoyl-3-[(E)-4-[5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]benzimidazol-4-yl]oxypropyl]-4-piperidyl]-6,8-dihydro-5H-[1,2,4]triazolo[4,3-a]pyrazine-7-carboxylate:

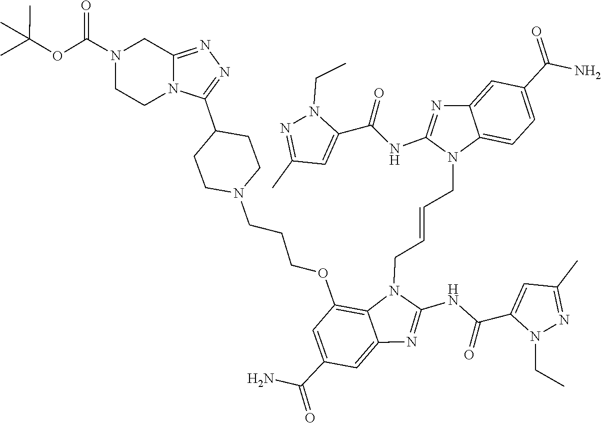 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)OC(C)(C)C)C5)CC3)c21]

A mixture of 3-[6-carbamoyl-3-[(E)-4-[5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]benzimidazol-4-yl]oxypropyl methanesulfonate (73.1 mg, 0.0882 mmol), tert-butyl 3-(4-piperidyl)-6,8-dihydro-5H-[1,2,4]triazolo[4,3-a]pyrazine-7-carboxylate (54.2 mg, 0.176 mmol), DIEA (114 mg, 0.882 mmol, 0.154 mL) and sodium iodide (13.2 mg, 0.882 mmol) in DMSO (2.5 mL) was stirred at 60° C. for 18 hours. The reaction mixture was filtered and the filtrate was purified by reverse phase preparative HPLC (acetonitrile/water+0.1% formic acid) to afford tert-butyl 3-[1-[3-[6-carbamoyl-3-[(E)-4-[5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]benzimidazol-4-yl]oxypropyl]-4-piperidyl]-6,8-dihydro-5H-[1,2,4]triazolo[4,3-a]pyrazine-7-carboxylate as a white solid (54 mg, 59%). LCMS (ESI) m/z 1040.4 (M+H).

(E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-(4-(5,6,7,8-tetrahydro-[1,2,4]triazolo[4,3-a]pyrazin-3-yl)piperidin-1-yl)propoxy)-1H-benzo[d]imidazole-5-carboxamide (Compound 64): To a mixture of tert-butyl 3-[1-[3-[6-carbamoyl-3-[(E)-4-[5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]benzimidazol-4-yl]oxypropyl]-4-piperidyl]-6,8-dihydro-5H-[1,2,4]triazolo[4,3-a]pyrazine-7-carboxylate (18.1 mg, 0.0174 mmol) in MeOH (2 mL) was added 4.0 M HCl in 1,4-dioxane (0.26 mL, 1.044 mmol). The reaction mixture was stirred at room temperature for 4 hours, and then concentrated. The residue was dissolved in DMSO (2 mL) and 28-30% NH₃·H₂O (0.1 mL), and then purified by reverse phase preparative HPLC (acetonitrile/water+0.1% 28-30% NH₃·H₂O) to afford (E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-(4-(5,6,7,8-tetrahydro-[1,2,4]triazolo[4,3-a]pyrazin-3-yl)piperidin-1-yl)propoxy)-1H-benzo[d]imidazole-5-carboxamide (Compound 60) as a white solid, 10.1 mg. LCMS (ESI) m/z 940.6 (M+H). ¹H NMR (400 MHz, DMSO) δ 12.79 (s, 2H), 7.99 (d, J=1.6 Hz, 1H), 7.93 (d, J=8.6 Hz, 2H), 7.70 (dd, J=8.4, 1.6 Hz, 1H), 7.64 (s, 1H), 7.41 (d, J=8.3 Hz, 1H), 7.29 (s, 3H), 6.54 (d, J=16.9 Hz, 2H), 6.07-5.95 (m, 1H), 5.78 (s, 1H), 5.82-5.72 (m, OH), 4.96 (d, J=5.4 Hz, 2H), 4.82 (d, J=5.7 Hz, 2H), 4.61-4.46 (m, 4H), 3.99 (t, J=6.1 Hz, 2H), 3.90 (s, 2H), 3.78 (t, J=5.5 Hz, 2H), 3.03 (t, J=5.5 Hz, 2H), 2.76 (d, J=10.7 Hz, 2H), 2.56 (dd, J=10.3, 5.9 Hz, 1H), 2.30 (t, J=7.2 Hz, 2H), 2.12 (d, J=14.2 Hz, 5H), 1.90 (s, 1H), 1.87-1.62 (m, 7H), 1.33-1.20 (m, 6H).

### Synthesis 2N. Compound 41

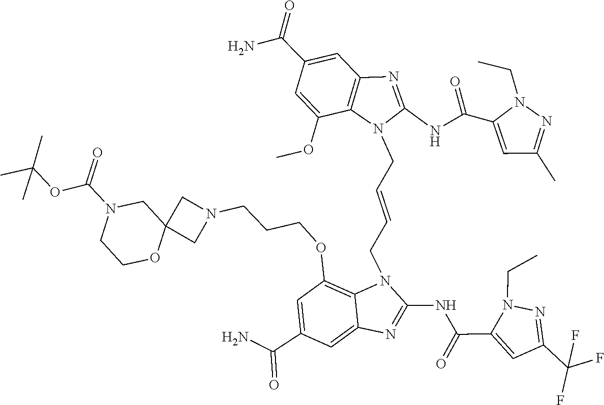 [SMILES: CCn1nc(C)cc1C(=O)Nc1nc2cc(C(N)=O)cc(OC)c2n1C/C=C/Cn1c(NC(=O)c2cc(C(F)(F)F)nn2CC)nc2cc(C(N)=O)cc(OCCCN3CC4(C3)CN(C(=O)OC(C)(C)C)CCO4)c21]

To a mixture of Compound I-1-12 (30 mg, 0.026 mmol) in DMF (2 mL) was added 1-ethyl-3-(trifluoromethyl)pyrazole-5-carbonyl isothiocyanate. The reaction mixture was stirred at R^{T} for 0.5 h, then HBTU (10.2 mg, 0.027 mmol) and DIEA (0.013 mL, 0.077 mmol) were added. Continue stirring at R^{T} for 0.5 h. Desired product formed as a major peak by LCMS. The reaction mixture was purified buy reverse phase HPLC (acetonitrile/water+0.1% formic acid) to afford tert-butyl (E)-2-(3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-(trifluoromethyl)-1H-pyrazole-5-carboxamido)-1H-benzo[d]imidazol-7-yl)oxy)propyl)-5-oxa-2,8-diazaspiro[3.5]nonane-8-carboxylate as its formic acid salt. (Compound 40, white solid, 18 mg, 67%). LC-MS [ESI]: Calculated for C₄₉H₆₀F₃N₁₄O₉ [M+H⁺]: 1045.45, Found: 1045.4.

### Preparation of Compound 41

### (E)-7-(3-(5-oxa-2,8-diazaspiro[3.5]nonan-2-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-(trifluoromethyl)-1H-pyrazole-5-carboxamido)-1H-benzo[d]imidazole-5-carboxamide (Compound 41)

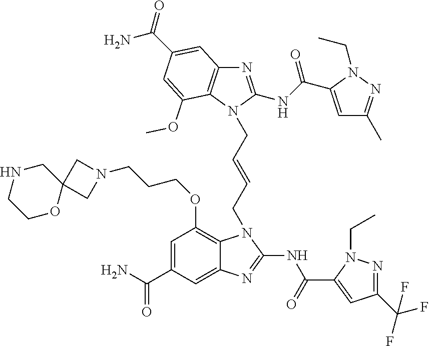 [SMILES: CCn1nc(C)cc1C(=O)Nc1nc2cc(C(N)=O)cc(OC)c2n1C/C=C/Cn1c(NC(=O)c2cc(C(F)(F)F)nn2CC)nc2cc(C(N)=O)cc(OCCCN3CC4(CNCCO4)C3)c21]

tert-butyl (E)-2-(3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-(trifluoromethyl)-1H-pyrazole-5-carboxamido)-1H-benzo[d]imidazol-7-yl)oxy)propyl)-5-oxa-2,8-diazaspiro[3.5]nonane-8-carboxylate (18 mg, 0.017 mmol) in 2 mL of 5% (v/v) trifluoracetic acid in 1,1,1,3,3,3-Hexafluoroisopropanol (HFIP) was stirred at room temperature for 1 hour. The reaction mixture was concentrated. The residue was diluted with DMSO (2 mL) and purified by reverse phase preparative HPLC (acetonitrile/water+0.1% TFA) to afford (E)-7-(3-(5-oxa-2,8-diazaspiro[3.5]nonan-2-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-(trifluoromethyl)-1H-pyrazole-5-carboxamido)-1H-benzo[d]imidazole-5-carboxamide (41) as its TFA salt (white solid, 17 mg, 84%). LC-MS [ESI]: Calculated for C₄₄H₅₂F₃N₁₄O₇ [M+H⁺]: 945.40, Found: 945.4. ¹H NMR (400 MHz, DMSO) δ 13.03 (s, 1H), 12.82 (s, 1H), 10.52 (s, 1H), 9.13 (s, 2H), 7.98 (s, 2H), 7.68 (s, 1H), 7.63 (s, 1H), 7.40 (d, J=11.7 Hz, 2H), 7.31 (d, J=9.2 Hz, 2H), 7.14 (s, 1H), 6.50 (s, 1H), 5.79 (s, 1H), 5.73 (d, J=16.3 Hz, 1H), 4.97 (s, 2H), 4.88 (d, J=5.0 Hz, 2H), 4.69 (q, J=7.2 Hz, 2H), 4.51 (d, J=7.2 Hz, 2H), 4.35 (s, 1H), 4.12 (s, 1H), 4.02 (s, 3H), 3.87 (s, 1H), 3.76 (s, 1H), 3.72-3.67 (m, 3H), 3.33 (s, 2H), 3.26 (s, 2H), 3.10 (d, J=17.1 Hz, 3H), 2.10 (s, 3H), 1.75 (s, 2H), 1.35 (t, J=7.1 Hz, 3H), 1.26 (t, J=7.1 Hz, 3H).

### Synthesis 2O Compound 77

### (E)-N-(7-(3-(5-oxa-2,8-diazaspiro[3.5]nonan-2-yl)propoxy)-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)-4-ethyl-2-methylthiazole-5-carboxamide (77)

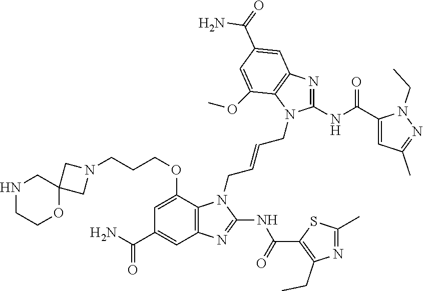 [SMILES: CCc1nc(C)sc1C(=O)Nc1nc2cc(C(N)=O)cc(OCCCN3CC4(CNCCO4)C3)c2n1C/C=C/Cn1c(NC(=O)c2cc(C)nn2CC)nc2cc(C(N)=O)cc(OC)c21]

Compound 77 was made similarly according to preparation of Compound 41 from Compound I-1-12 and 4-ethyl-2-methylthiazole-5-carbonyl isothiocyanate. LC-MS [ESI]: Calculated for C₄₄H₅₄N₁₃O₇S [M+H]⁺: 908.39, Found: 908.4. ¹H NMR (400 MHz, DMSO) δ 12.82 (s, 2H), 10.56 (s, 1H), 9.16 (s, 2H), 7.96 (s, 2H), 7.63 (s, 2H), 7.38 (s, 2H), 7.31 (d, J=8.6 Hz, 2H), 6.51 (s, 1H), 5.81 (s, 2H), 4.89 (s, 4H), 4.52 (q, J=7.2 Hz, 2H), 4.07 (s, 3H), 3.92 (d, J=31.9 Hz, 2H), 3.32 (d, J=19.0 Hz, 2H), 3.10 (q, J=7.5 Hz, 4H), 2.10 (s, 3H), 1.87 (s, 1H), 1.26 (q, J=7.1 Hz, 3H), 1.14 (t, J=7.5 Hz, 3H).

### Synthesis 2P. Compound 78

### (E)-7-(3-(5-oxa-2,8-diazaspiro[3.5]nonan-2-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-methyl-1H-pyrazole-5-carboxamido)-1H-benzo[d]imidazole-5-carboxamide (78)

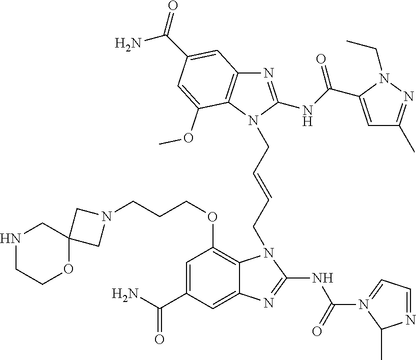

Compound 78 was made similarly according to preparation of Compound 41 from Compound I-1-12 and 1-methyl-1H-pyrazole-5-carbonyl isothiocyanate. LC-MS [ESI]: Calculated for C₄₂H₅₁N₁₄O₇ [M+H⁺]: 863.40, Found: 863.4. ¹H NMR (400 MHz, DMSO) δ 8.24-8.15 (m, 1H), 7.98 (s, 2H), 7.65 (dd, J=4.0, 1.3 Hz, 1H), 7.41-7.22 (m, 4H), 6.76 (d, J=1.9 Hz, 1H), 6.52 (s, 1H), 5.83 (q, J=4.2 Hz, 1H), 4.98-4.88 (m, 3H), 4.52 (q, J=7.1 Hz, 2H), 4.15 (s, 2H), 3.94 (t, J=6.2 Hz, 2H), 3.73 (s, 3H), 3.38 (d, J=4.9 Hz, 9H), 3.20 (d, J=7.2 Hz, 3H), 2.75 (s, 2H), 2.61 (d, J=7.0 Hz, 3H), 2.41 (t, J=6.9 Hz, 2H), 2.11 (s, 2H), 1.53 (t, J=6.7 Hz, 2H), 1.26 (q, J=7.6 Hz, 3H).

### Synthesis 2Q. Compound 79

### (E)-N-(7-(3-(5-oxa-2,8-diazaspiro[3.5]nonan-2-yl)propoxy)-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)-2,4-dimethylthiazole-5-carboxamide (79)

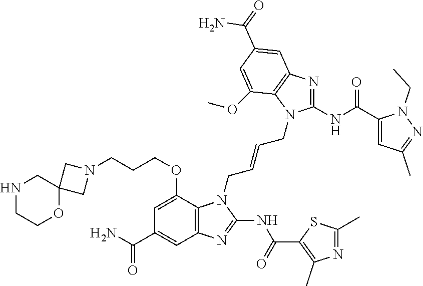 [SMILES: CCn1nc(C)cc1C(=O)Nc1nc2cc(C(N)=O)cc(OC)c2n1C/C=C/Cn1c(NC(=O)c2sc(C)nc2C)nc2cc(C(N)=O)cc(OCCCN3CC4(CNCCO4)C3)c21]

Compound 79 was made similarly according to preparation of Compound 41 from Compound I-1-12 and 2,4-dimethylthiazole-5-carbonyl isothiocyanate. LC-MS [ESI]: Calculated for C₄₃H₅₂N₁₃O₇S [M+H⁺]: 894.38, Found: 894.4. ¹H NMR (400 MHz, DMSO) δ 7.95 (s, 2H), 7.63 (dd, J=12.4, 1.3 Hz, 2H), 7.29 (dd, J=10.1, 1.4 Hz, 2H), 6.51 (s, 1H), 5.85 (d, J=2.9 Hz, 2H), 4.89 (s, 4H), 4.53 (d, J=7.2 Hz, 1H), 3.97 (s, 2H), 3.73 (s, 3H), 3.17 (d, J=7.4 Hz, 2H), 2.69 (s, 2H), 2.65-2.53 (m, 7H), 2.41 (t, J=6.6 Hz, 1H), 2.10 (s, 3H), 1.60-1.52 (m, 2H), 1.26 (q, J=8.1 Hz, 3H).

### Synthesis 2R. Compound 80

### (E)-N-(7-(3-(5-oxa-2,8-diazaspiro[3.5]nonan-2-yl)propoxy)-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)-2-ethyl-4-methylthiazole-5-carboxamide (80)

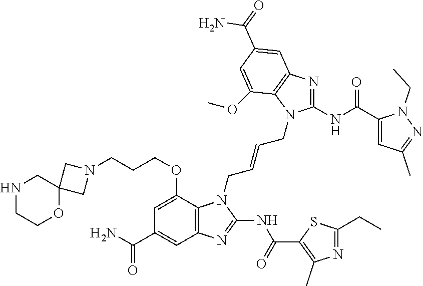 [SMILES: CCc1nc(C)c(C(=O)Nc2nc3cc(C(N)=O)cc(OCCCN4CC5(CNCCO5)C4)c3n2C/C=C/Cn2c(NC(=O)c3cc(C)nn3CC)nc3cc(C(N)=O)cc(OC)c32)s1]

Compound 80 was made similarly according to preparation of Compound 41 from Compound I-1-12 and 2-ethyl-4-methylthiazole-5-carbonyl isothiocyanate. LC-MS [ESI]: Calculated for C₄₄H₅₄N₁₃O₇S [M+H⁺]: 908.39, Found: 908.4. ¹H NMR (400 MHz, DMSO) δ 7.95 (s, 2H), 7.63 (dd, J=11.0, 1.3 Hz, 2H), 7.36-7.25 (m, 4H), 6.51 (s, 1H), 5.86 (s, 2H), 4.89 (s, 4H), 4.52 (q, J=7.2 Hz, 2H), 3.95 (d, J=6.5 Hz, 2H), 3.72 (s, 3H), 3.17 (d, J=7.3 Hz, 2H), 2.83 (q, J=7.5 Hz, 2H), 2.69 (s, 2H), 2.64 (s, 2H), 2.58 (d, J=7.7 Hz, 2H), 2.41 (t, J=6.8 Hz, 1H), 2.09 (s, 3H), 1.55 (d, J=7.8 Hz, 2H), 1.32-1.19 (m, 6H).

### Synthesis 2R. Compound 81

### (E)-7-(3-(5-oxa-2,8-diazaspiro[3.5]nonan-2-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-methyl-3-(trifluoromethyl)-1H-pyrazole-5-carboxamido)-1H-benzo[d]imidazole-5-carboxamide(81)

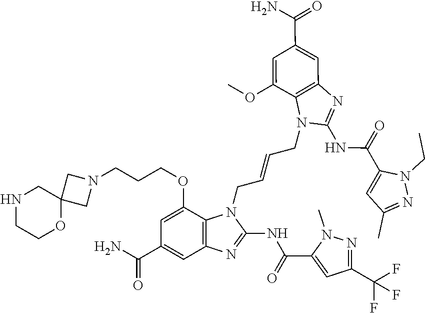 [SMILES: CCn1nc(C)cc1C(=O)Nc1nc2cc(C(N)=O)cc(OC)c2n1C/C=C/Cn1c(NC(=O)c2cc(C(F)(F)F)nn2C)nc2cc(C(N)=O)cc(OCCCN3CC4(CNCCO4)C3)c21]

Compound 81 was made similarly according to preparation of Compound 41 from Compound I-1-12 and 1-methyl-3-(trifluoromethyl)-1H-pyrazole-5-carbonyl isothiocyanate. LC-MS [ESI]: Calculated for C₄₃H₅₀F₃N₁₄O₇ [M+H]⁺: 931.39, Found: 931.6. ¹H NMR (400 MHz, DMSO) δ 7.97 (d, J=8.2 Hz, 2H), 7.66 (dd, J=4.6, 1.3 Hz, 2H), 7.36-7.25 (m, 4H), 7.19 (s, 1H), 6.50 (s, 1H), 5.91-5.74 (m, 2H), 4.98 (d, J=4.4 Hz, 2H), 4.89 (d, J=4.5 Hz, 2H), 4.52 (q, J=7.1 Hz, 2H), 4.24 (s, 3H), 3.91 (t, J=6.2 Hz, 2H), 3.71 (s, 3H), 3.18-3.11 (m, 2H), 2.68 (s, 2H), 2.59-2.51 (m, 5H), 2.36 (t, J=7.0 Hz, 2H), 2.09 (s, 3H), 1.53-1.45 (m, 2H), 1.27 (t, J=7.1 Hz, 3H).

### Synthesis 2S. Compound 88

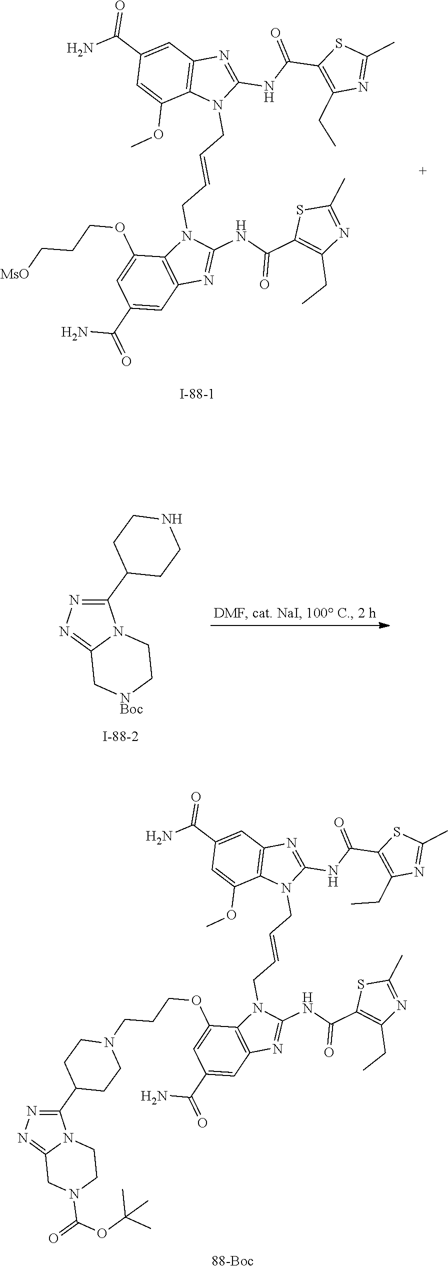 [SMILES: CCc1nc(C)sc1C(=O)Nc1nc2cc(C(N)=O)cc(OC)c2n1C/C=C/Cn1c(NC(=O)c2sc(C)nc2CC)nc2cc(C(N)=O)cc(OCCCOS(C)(=O)=O)c21 ; CC(C)(C)OC(=O)N1CCn2c(nnc2C2CCNCC2)C1 ; CCc1nc(C)sc1C(=O)Nc1nc2cc(C(N)=O)cc(OC)c2n1C/C=C/Cn1c(NC(=O)c2sc(C)nc2CC)nc2cc(C(N)=O)cc(OCCCN3CCC(c4nnc5n4CCN(C(=O)OC(C)(C)C)C5)CC3)c21]

### Synthesis of tert-butyl 3-[1-[3-[6-carbamoyl-3-[(E)-4-[5-carbamoyl-2-[(4-ethyl-2-methyl-thiazole-5-carbonyl)amino]-7-methoxy-benzimidazol-1-yl]but-2-enyl]-2-[(4-ethyl-2-methyl-thiazole-5-carbonyl)amino]benzimidazol-4-yl]oxypropyl]-4-piperidyl]-6,8-dihydro-5H-[1,2,4]triazolo[4,3-a]pyrazine-7-carboxylate (88-Boc)

To a stirred solution of (E)-3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl methanesulfonate (I-88-1, 300 mg, 0.349 mmol, 1 equiv.) in N,N-dimethylformamide (6.000 mL, 0.384 mmol) were added tert-butyl 3-(piperidin-4-yl)-5,6-dihydro-[1,2,4]triazolo[4,3-a]pyrazine-7 (8H)-carboxylate (I-88-2, 118.102 mg, 0.384 mmol, 1.1 equiv.) and Sodium iodide (5.235 mg, 0.035 mmol, 0.1 equiv.) at room temperature for 5 minutes. The reaction mixture was heated to 80° C. for 12 hours. The reaction was monitored by TLC and LCMS. The reaction mixture was added cold water (10 mL), and extracted with MeOH in DCM (10%, 100 mL×4), the organic phases were combined, dried over anhydrous sodium sulphate and filtered, the filtrate was concentrated under reduced pressure, and the crude compound was purified by Pre-HPLC (YMC ODS AQ C18 (20×250 mm), 5 μm Flow: 15.0 mL/min and using 25-90% ACN: 0.1% TFA in water as eluent, Flow rate: 15 mL/min, run time: 25 mins to afford tert-butyl 3-[-[3-[6-carbamoyl-3-[(E)-4-[5-carbamoyl-2-[(4-ethyl-2-methyl-thiazole-5-carbonyl)amino]-7-methoxy-benzimidazol-1-yl]but-2-enyl]-2-[(4-ethyl-2-methyl-thiazole-5-carbonyl) amino]benzimidazol-4-yl]oxypropyl]-4-piperidyl]-6,8-dihydro-5H-[1,2,4]triazolo[4,3-a]pyrazine-7-carboxylate (Compound 88-Boc, 0.019 g, 0.017 mmol, 9%) as an off white solid. LC-MS [ESI]: Calculated for C₅₃H₆₅N₁₅O₈S₂ [M+H⁺]: 1104.47, Found: 1104.63.

### (E)-N-(5-carbamoyl-1-(4-(5-carbamoyl-2-(4-ethyl-2-methylthiazole-5-carboxamido)-7-(3-(4-(5,6,7,8-tetrahydro-[1,2,4]triazolo[4,3-a]pyrazin-3-yl)piperidin-1-yl)propoxy)-1H-benzo[d]imidazol-1-yl)but-2-en-1-yl)-7-methoxy-1H-benzo[d]imidazol-2-yl)-4-ethyl-2-methylthiazole-5-carboxamide (88)

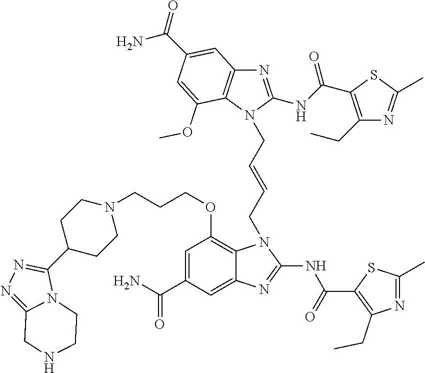 [SMILES: CCc1nc(C)sc1C(=O)Nc1nc2cc(C(N)=O)cc(OC)c2n1C/C=C/Cn1c(NC(=O)c2sc(C)nc2CC)nc2cc(C(N)=O)cc(OCCCN3CCC(c4nnc5n4CCNC5)CC3)c21]

Compound 88-Boc (17.8 mg, 0.016 mmol) in 2 mL of 5% (v/v) trifluoracetic acid in 1,1,1,3,3,3-Hexafluoroisopropanol (HFIP) was stirred at room temperature for 1.5 hour. The reaction mixture was concentrated. The residue was diluted with DMSO (2 mL) and purified by reverse phase preparative HPLC (acetonitrile/water+0.1% TFA) to afford (E)-N-(5-carbamoyl-1-(4-(5-carbamoyl-2-(4-ethyl-2-methylthiazole-5-carboxamido)-7-(3-(4-(5,6,7,8-tetrahydro-[1,2,4]triazolo[4,3-a]pyrazin-3-yl)piperidin-1-yl)propoxy)-1H-benzo[d]imidazol-1-yl)but-2-en-1-yl)-7-methoxy-1H-benzo[d]imidazol-2-yl)-4-ethyl-2-methylthiazole-5-carboxamide (88) as its TFA salt (white solid, 10.3 mg, 52%). LC-MS [ESI]: Calculated for C₄₈H₅₈N₁₅O₆S₂ [M+H⁺]: 1000.41, Found: 1000.4. ¹H NMR (400 MHz, DMSO) δ 12.82 (d, J=7.9 Hz, 2H), 9.53 (s, 2H), 8.01 (s, 1H), 7.95 (s, 1H), 7.66-7.55 (m, 2H), 7.38 (s, 2H), 7.36-7.27 (m, 2H), 5.91-5.73 (m, 2H), 4.87 (dd, J=16.5, 4.9 Hz, 4H), 4.55 (s, 2H), 4.18 (t, J=5.8 Hz, 2H), 4.04 (t, J=5.9 Hz, 2H), 3.78 (s, 3H), 3.66 (s, 2H), 3.13 (s, 3H), 3.18-2.98 (m, 4H), 2.89 (q, J=13.4 Hz, 2H), 2.50 (d, J=17.4 Hz, 5H), 2.06 (d, J=13.7 Hz, 2H), 2.00-1.89 (m, 2H), 1.21-1.12 (m, 6H).

### Synthesis 2T. Compound 40

### (E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-(8-(methyl-L-leucyl)-5-oxa-2,8-diazaspiro[3.5]nonan-2-yl)propoxy)-1H-benzo[d]imidazole-5-carboxamide (40)

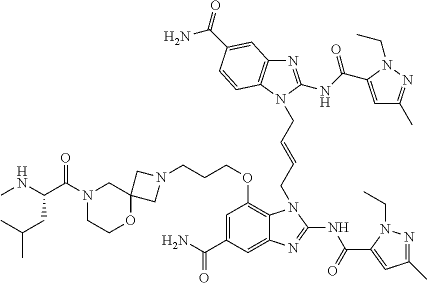

Compound 40 was made similarly according to preparation of Compound 2 from Compound 1 and Fmoc-N-Me-Leu-OH. LC-MS [ESI]: Calculated for C₅₀H₆₆N₁₅O₇ [M+H⁺]: 988.52, Found: 988.6. ¹H NMR (400 MHz, DMSO) δ 7.97 (d, J=1.6 Hz, 1H), 7.92 (s, 1H), 7.69 (d, J=8.4 Hz, 1H), 7.64 (s, 1H), 7.43-7.35 (m, 1H), 7.30 (s, 2H), 6.53 (d, J=2.9 Hz, 2H), 6.02-5.92 (m, 1H), 5.73 (d, J=15.5 Hz, 1H), 4.94 (s, 2H), 4.81 (d, J=5.7 Hz, 2H), 4.52 (d, J=7.1 Hz, 4H), 4.00 (s, 2H), 3.75 (d, J=13.0 Hz, 1H), 3.55 (d, J=9.7 Hz, 1H), 3.44 (d, J=10.9 Hz, 5H), 3.17 (d, J=19.0 Hz, 2H), 2.93 (d, J=6.0 Hz, 2H), 2.63 (t, J=6.8 Hz, 2H), 2.44 (t, J=6.6 Hz, 1H), 2.16-2.08 (m, 3H), 2.11 (s, 5H), 1.68 (q, J=6.6 Hz, 1H), 1.59 (s, 6H), 1.31-1.24 (m, 6H), 1.20 (s, 2H), 0.88-0.74 (m, 6H).

### Synthesis 2U. Compound 82

### (E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-(8-(methylglycyl)-5-oxa-2,8-diazaspiro[3.5]nonan-2-yl)propoxy)-1H-benzo[d]imidazole-5-carboxamide (82)

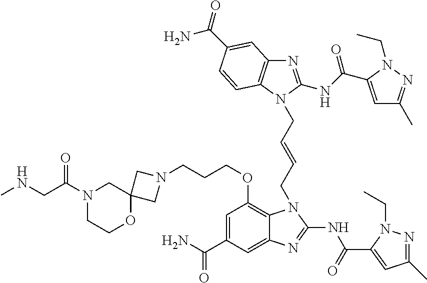

Compound 82 was made similarly according to preparation of Compound 2 from Compound 1 and Fmoc-Sar-OH. LC-MS [ESI]: Calculated for C₄₆H₅₈N₁₅O₇ [M+H⁺]: 932.46, Found: 932.6. ¹H NMR (400 MHz, DMSO) δ 7.97 (d, J=1.6 Hz, 1H), 7.89 (s, 2H), 7.70-7.60 (m, 2H), 7.34 (d, J=8.3 Hz, 1H), 7.25 (s, 3H), 6.50 (s, 2H), 5.95 (dd, J=13.3, 7.7 Hz, 1H), 5.73 (dd, J=14.1, 7.3 Hz, 1H), 4.93 (s, 2H), 4.78 (d, J=5.7 Hz, 2H), 4.53 (p, J=7.0 Hz, 4H), 3.99 (s, 2H), 3.52 (d, J=11.9 Hz, 1H), 3.46-3.41 (m, 7H), 3.25 (s, 1H), 3.12 (d, J=7.1 Hz, 1H), 2.67-2.57 (m, 3H), 2.43 (s, 2H), 2.26 (d, J=1.9 Hz, 5H), 2.12 (d, J=3.9 Hz, 6H), 1.63-1.53 (m, 4H), 1.40 (d, J=5.3 Hz, 2H), 1.31-1.22 (m, 6H).

### Synthesis 2V. Compound 83

### (E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-(4-(7-(methylglycyl)-5,6,7,8-tetrahydro-[1,2,4]triazolo[4,3-a]pyrazin-3-yl)piperidin-1-yl)propoxy)-1H-benzo[d]imidazole-5-carboxamide (83)

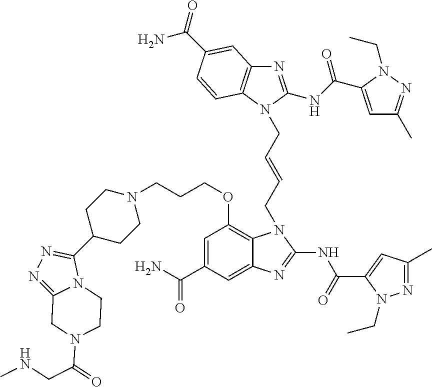

Compound 83 was made similarly according to preparation of Compound 2 from Compound 89 and Fmoc-Sar-OH. LC-MS [ESI]: Calculated for C₅₀H₆₃N₁₈O₆ [M+H⁺]: 1011.51, Found: 1011.6. ¹H NMR (400 MHz, DMSO) δ 8.90 (s, 2H), 7.98 (d, J=16.5 Hz, 3H), 7.35 (s, 2H), 4.96 (s, 2H), 4.85 (d, J=9.5 Hz, 4H), 4.52 (dt, J=13.4, 6.9 Hz, 3H), 4.23 (s, 2H), 4.10 (s, 11H), 4.01 (s, 1H), 3.01 (s, 1H), 2.88 (s, 2H), 2.59 (s, 2H), 2.12 (d, J=8.2 Hz, 6H), 2.05 (s, 5H), 1.33-1.21 (m, 6H).

### Synthesis of Compound 89

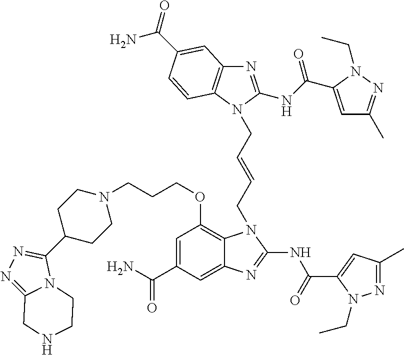 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCNC5)CC3)c21]

Compound 89 was made similarly according to preparation of Compound 1. 1H NMR (400 MHz, DMSO) δ 12.84 (s, 2H), 9.84 (s, 2H), 7.97 (dd, J=14.6, 1.6 Hz, 1H), 7.75 (dd, J=8.4, 1.6 Hz, 1H), 7.67 (s, 1H), 7.45 (d, J=8.4 Hz, 1H), 7.37-7.32 (m, 2H), 6.55-6.48 (m, 2H), 6.05-5.95 (m, 1H), 4.96 (s, 2H), 4.82 (d, J=5.6 Hz, 2H), 4.55 (s, 1H), 4.52 (s, 5H), 4.49 (d, J=6.5 Hz, 1H), 4.22 (d, J=6.0 Hz, 2H), 4.11 (d, J=6.1 Hz, 2H), 3.64 (s, 2H), 3.49 (s, 1H), 3.12 (s, 2H), 3.05 (s, 1H), 2.89 (s, 2H), 2.22 (s, 1H), 2.15-2.01 (m, 12H), 1.33-1.20 (m, 6H).

### Synthesis 2W. Compound 84

### (E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-(4-(7-glycyl-5,6,7,8-tetrahydro-[1,2,4]triazolo[4,3-a]pyrazin-3-yl)piperidin-1-yl)propoxy)-1H-benzo[d]imidazole-5-carboxamide (84)

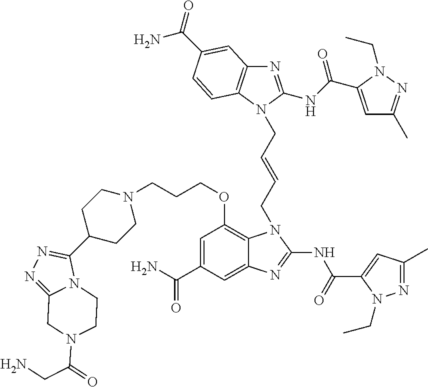 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)CN)C5)CC3)c21]

Compound 84 was made similarly according to preparation of Compound 2 Compound 89 and Fmoc-Gly-OH. LC-MS [ESI]: Calculated for C₄₉H₆₁N₁₈O₆ [M+H⁺]: 997.49, Found: 997.6. ¹H NMR (400 MHz, DMSO) δ 10.80 (s, 1H), 8.27 (t, J=17.0 Hz, 3H), 7.98 (dd, J=15.9, 3.0 Hz, 1H), 7.77 (t, J=7.7 Hz, 1H), 7.67 (d, J=4.9 Hz, 1H), 7.47 (t, J=7.7 Hz, 1H), 7.36 (s, 2H), 6.56-6.48 (m, 2H), 6.02 (d, J=17.1 Hz, 1H), 5.69 (d, J=15.6 Hz, 1H), 4.97 (s, 2H), 4.90 (s, 2H), 4.83 (d, J=5.6 Hz, 2H), 4.51 (dt, J=13.2, 6.9 Hz, 4H), 4.16 (s, 1H), 4.10 (s, 3H), 4.02 (s, 3H), 3.94 (s, 1H), 3.73-3.63 (m, OH), 3.11 (s, 3H), 3.03-2.77 (m, 3H), 2.19-2.05 (m, 9H), 1.65 (d, J=5.6 Hz, 1H), 1.32-1.21 (m, 6H).

### Synthesis 2X. Compound 85

### (E)-3-(4-(5-carbamoyl-2-(1-ethyl-3-methyl-1H-pyrazole-5-carboxamido)-7-(3-(4-(7-(methylglycyl)-5,6,7,8-tetrahydro-[1,2,4]triazolo[4,3-a]pyrazin-3-yl)piperidin-1-yl)propoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (85)

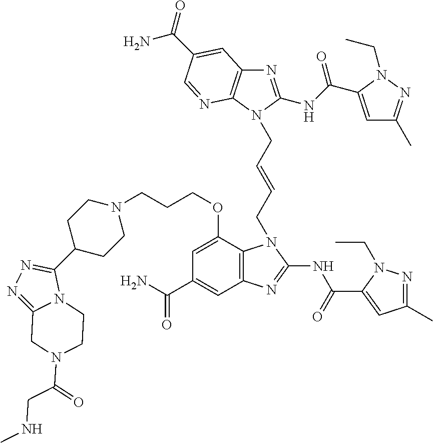

Compound 85 was made similarly according to preparation of Compound 2 from Compound 21 and Fmoc-Sar-OH. LC-MS [ESI]: Calculated for C₄₉H₆₂N₁₉O₆ [M+H⁺]: 1012.51, Found: 1012.6. ¹H NMR (400 MHz, DMSO) δ 8.70 (d, J=1.9 Hz, 1H), 8.16-8.10 (m, 2H), 7.95 (s, 1H), 7.63 (s, 1H), 7.51 (s, 1H), 7.30 (d, J=7.2 Hz, 2H), 6.55 (s, 1H), 6.49 (s, 1H), 5.93 (dt, J=15.9, 5.4 Hz, 1H), 5.83 (dt, J=15.7, 5.6 Hz, 1H), 4.94 (d, J=5.2 Hz, 2H), 4.82 (s, 1H), 4.80-4.73 (m, 4H), 4.52 (dq, J=24.2, 7.2 Hz, 4H), 4.02 (t, J=6.1 Hz, 3H), 3.89 (d, J=7.2 Hz, 3H), 3.46 (d, J=16.8 Hz, 2H), 2.93-2.86 (m, 3H), 2.77 (d, J=10.6 Hz, 2H), 2.65-2.55 (m, 1H), 2.40 (s, 1H), 2.30 (t, J=4.4 Hz, 5H), 2.11 (d, J=14.8 Hz, 6H), 1.82 (t, J=11.2 Hz, 2H), 1.74 (s, 1H), 1.73 (s, 4H), 1.67 (t, J=12.9 Hz, 1H), 1.55 (s, 6H), 1.33-1.22 (m, 6H).

### Synthesis 2Y. Compound 57

### Preparation of tert-butyl (S,E)-(1-(3-(1-(3-((5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (Boc-57)

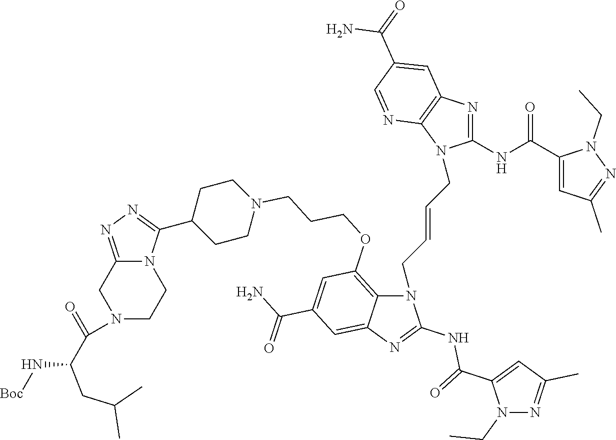 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)OC(C)(C)C)C5)CC3)c21]

To a mixture of Compound 21 (31.3 mg, 0.024 mmol), Boc-L-Leu-OH (6.8 mg, 0.029 mmol), and DIEA (0.021 mL, 0.122 mmol) in DMF (2 mL) was added HBTU. The reaction mixture was stirred at RT for 15 min then purified by reverse phase HPLC: (acetonitrile/water +0.1% formic acid) to afford tert-butyl (S,E)-(1-(3-(1-(3-((5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate as its formic acid salt (white solid, 15 mg, 53%). LC-MS [ESI]: Calculated for C₅₇H₇₆N₁₉O₈ [M+H⁺]: 1154.60, Found: 1154.6.

### (E)-3-(4-(7-(3-(4-(7-(L-leucyl)-5,6,7,8-tetrahydro-[1,2,4]triazolo[4,3-a]pyrazin-3-yl)piperidin-1-yl)propoxy)-5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (57)

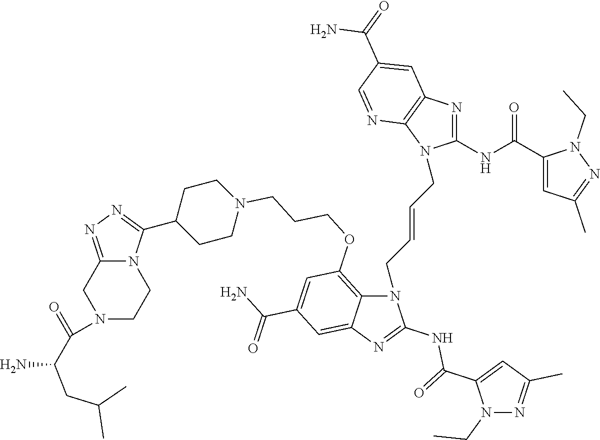 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)[C@@H](N)CC(C)C)C5)CC3)c21]

tert-butyl (S,E)-(1-(3-(1-(3-((5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate in 2 mL of 5% (v/v) trifluoracetic acid in 1,1,1,3,3,3-Hexafluoroisopropanol (HFIP) was stirred at room temperature for 0.5 hour. The reaction mixture was concentrated. The residue was diluted with DMSO (2 mL) and purified by reverse phase preparative HPLC (acetonitrile/water+0.1% ammonium hydroixde) to afford (E)-3-(4-(7-(3-(4-(7-(L-leucyl)-5,6,7,8-tetrahydro-[1,2,4]triazolo[4,3-a]pyrazin-3-yl)piperidin-1-yl)propoxy)-5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (57) (white solid, 17.2 mg, 68%). LC-MS [ESI]: Calculated for C₅₂H₆₈N₁₉O₆ [M+H⁺]: 1054.55, Found: 1054.6. ¹H NMR (400 MHz, DMSO) δ 8.71 (d, J=1.8 Hz, 1H), 8.17-8.10 (m, 1H), 7.95 (s, 1H), 7.63 (s, 1H), 7.52 (s, 1H), 7.31 (d, J=10.3 Hz, 2H), 6.53 (d, J=13.8 Hz, 2H), 5.98-5.89 (m, 1H), 5.86 (s, 1H), 4.92 (d, J=17.2 Hz, 2H), 4.78 (s, 2H), 4.51 (dt, J=21.5, 7.2 Hz, 4H), 4.03 (s, 5H), 3.89 (s, 1H), 2.80 (s, 2H), 2.11 (d, J=12.6 Hz, 6H), 1.84 (s, 2H), 1.74 (s, 7H), 1.40 (s, 2H), 1.33-1.22 (m, 6H), 0.91 (dd, J=12.0, 6.8 Hz, 6H).

### Synthesis 2Z. Compound 43

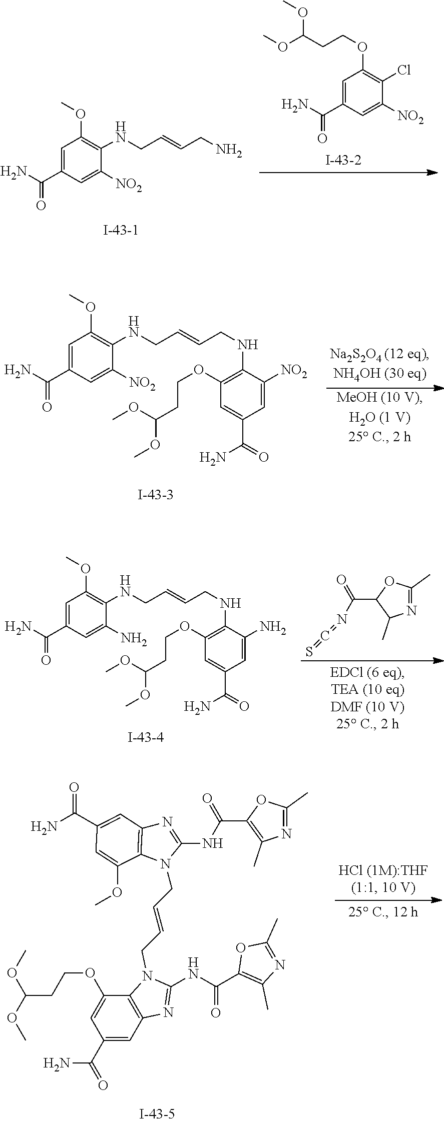

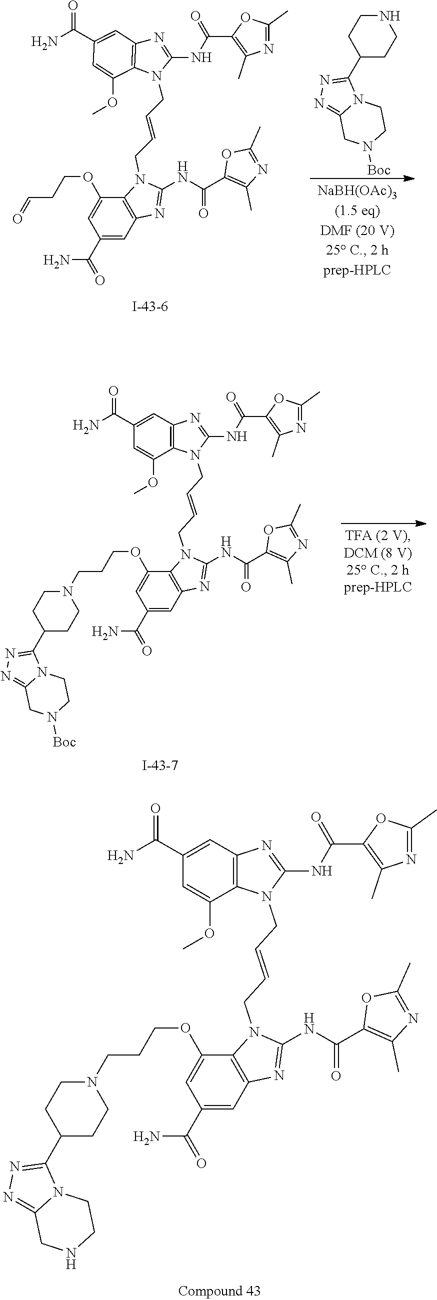 [SMILES: COc1cc(C(N)=O)cc2nc(NC(=O)c3oc(C)nc3C)n(C/C=C/Cn3c(NC(=O)c4oc(C)nc4C)nc4cc(C(N)=O)cc(OCCC=O)c43)c12 ; CC(C)(C)OC(=O)N1CCn2c(nnc2C2CCNCC2)C1 ; COc1cc(C(N)=O)cc2nc(NC(=O)c3oc(C)nc3C)n(C/C=C/Cn3c(NC(=O)c4oc(C)nc4C)nc4cc(C(N)=O)cc(OCCCN5CCC(c6nnc7n6CCN(C(=O)OC(C)(C)C)C7)CC5)c43)c12 ; COc1cc(C(N)=O)cc2nc(NC(=O)c3oc(C)nc3C)n(C/C=C/Cn3c(NC(=O)c4oc(C)nc4C)nc4cc(C(N)=O)cc(OCCCN5CCC(c6nnc7n6CCNC7)CC5)c43)c12]

Synthesis of (E)-4-((4-((4-carbamoyl-2-(3,3-dimethoxypropoxy)-6-nitrophenyl)amino)but-2-en-1-yl)amino)-3-methoxy-5-nitrobenzamide (I-43-3): To a mixture of I-43-1 (3 g, 80% purity, 8.56 mmol) and I-43-2 (3.27 g, 10.28 mmol, 1.2 eq) in n-butanol (30 mL) was added diisopropylethylamine (5.53 g, 42.81 mmol, 5 eq) and sodium bicarbonate (1.44 g, 17.13 mmol, 2 eq). The reaction system was heated to 120° C. in a sealed tube and stirred for 16 hrs and then poured into water. The resulting solid was filtered and washed with water to afford (E)-4-((4-((4-carbamoyl-2-(3,3-dimethoxypropoxy)-6-nitrophenyl)amino)but-2-en-1-yl)amino)-3-methoxy-5-nitrobenzamide (2.7 g, 85% purity, 61% yield) as red solid. 1H NMR (400 MHz, DMSO-d₆) δ ppm 1.94-2.08 (m, 2H) 3.24 (s, 6H) 3.81 (s, 3H) 3.93-4.19 (m, 6H) 4.56 (t, J=5.60 Hz, 1H) 5.60 (br d, J=2.26 Hz, 2H) 7.32 (br s, 2H) 7.52 (s, 2H) 7.69 (br t, J=6.08 Hz, 2H) 8.04 (br s, 2H) 8.15 (s, 2H). LCMS (ESI+): m/z 585.0 (M+Na)+, R^{T}: 0.634 min.

Synthesis of (E)-3-amino-4-((4-((2-amino-4-carbamoyl-6-(3,3-dimethoxypropoxy)phenyl)amino)but-2-en-1-yl)amino)-5-methoxybenzamide (I-43-4): Compound I-43-3 (2.7 g, 4.08 mmol) was dissolved in methanol (27 mL), an aqueous solution (2.7 mL) of sodium dithionite (8.52 g, 48.96 mmol, 12 eq) and aqueous ammonia (7.72 g, 122.39 mmol, 8.48 mL, 27% aqueous solution, 30 eq) were serially added. The reaction system was stirred at 25° C. for 2 hrs. After the reaction was completed, the mixture was diluted with methanol (10 mL) and ethyl acetate (100 mL) and washed with saturated brine (30 mL×3). The organic phase was dried over anhydrous sodium sulfate and filtered, and the filtrate was concentrated under reduced pressure. The residue was purified by prep-HPLC (neutral condition) to give (E)-3-amino-4-((4-((2-amino-4-carbamoyl-6-(3,3-dimethoxypropoxy)phenyl)amino) but-2-en-1-yl)amino)-5-methoxybenzamide (800 mg, 37% yield, 95% purity) as white solid. 1H NMR (400 MHz, DMSO-d₆) δ ppm 1.98 (q, J=6.16 Hz, 2H) 3.25 (s, 6H) 3.42-3.60 (m, 4H) 3.73 (s, 3H) 3.75-3.89 (m, 2H) 3.96 (t, J=6.20 Hz, 2H) 4.56-4.71 (m, 5H) 5.66 (s, 2H) 6.77 (dd, J=3.70, 1.67 Hz, 2H) 6.85 (s, 2H) 6.96 (br s, 2H) 7.62 (br s, 2H). LCMS (ESI+): m/z 503.3 (M+1)+, R^{T}: 2.022 min.

Synthesis of (E)-N-(5-carbamoyl-1-(4-(5-carbamoyl-2-(2,4-dimethyloxazole-5-carboxamido)-7-methoxy-1H-benzo[d]imidazol-1-yl)but-2-en-1-yl)-7-(3,3-dimethoxypropoxy)-1H-benzo[d]imidazol-2-yl)-2,4-dimethyloxazole-5-carboxamide (I-43-5): Compound I-43-4 (500 mg, 945.16 mol, 1 eq) was dissolved in N, N-dimethylformamide (5 mL), and compound 6A (688.83 mg, 3.78 mmol, 4 eq) was added dropwise, then 1-ethyl-(3-dimethylaminopropyl)carbonyldiimine hydrochloride (1.09 g, 5.67 mmol, 6 eq) and triethylamine (956.40 mg, 9.45 mmol, 1.32 mL, 10 eq) were added. The reaction system was stirred at 25° C. for 2 hrs. The reaction was quenched by adding water (10 mL), and the resulting solid was filtered, washed with water (5 mL) and dried to afford (E)-N-(5-carbamoyl-1-(4-(5-carbamoyl-2-(2,4-dimethyloxazole-5-carboxamido)-7-methoxy-1H-benzo[d]imidazol-1-yl)but-2-en-1-yl)-7-(3,3-dimethoxypropoxy)-1H-benzo[d]imidazol-2-yl)-2,4-dimethyloxazole-5-carboxamide (540 mg, 66% yield, 92% purity) as white solid. 1H NMR (400 MHz, DMSO-d₆) δ ppm 1.76-1.92 (m, 2H) 2.26-2.33 (m, 6H) 2.38 (d, J=4.65 Hz, 6H) 3.16 (s, 6H) 3.76 (s, 3H) 4.01 (br t, J=6.24 Hz, 2H) 4.45 (t, J=5.56 Hz, 1H) 4.89 (br dd, J=13.14, 4.22 Hz, 4H) 5.69-5.90 (m, 2H) 7.25-7.40 (m, 4H) 7.64 (s, 2H) 7.98 (br d, J=4.40 Hz, 2H). LCMS (ESI+): m/z 799.2 (M+1)+, RT: 0.583 min.

Synthesis of (E)-N-(5-carbamoyl-1-(4-(5-carbamoyl-2-(2,4-dimethyloxazole-5-carboxamido)-7-(3-oxopropoxy)-1H-benzo[d]imidazol-1-yl)but-2-en-1-yl)-7-methoxy-1Hbenzo[d]imidazol-2-yl)-2,4-dimethyloxazole-5-carboxamide (I-43-6): Compound I-43-35 (300 mg, 345.52 μmol, 1 eq) was dissolved in tetrahydrofuran (1.5 mL), and hydrochloric acid aqueous solution (1.0 M, 1.5 mL) were serially added. The reaction system was stirred at 25° C. for 12 hrs and the resulting solid was filtered, washed with water (2 mL) and dried to give (E)-N-(5-carbamoyl-1-(4-(5-carbamoyl-2-(2,4-dimethyloxazole-5-carboxamido)-7-(3-oxopropoxy)-1H-benzo[d]imidazol-1-yl)but-2-en-1-yl)-7-methoxy-1Hbenzo[d]imidazol-2-yl)-2,4-dimethyloxazole-5-carboxamide (200 mg, 69% yield, 90% purity) as white solid. 1H NMR (400 MHz, DMSO-d₆) δ ppm 2.28 (s, 6H) 2.38 (s, 6H) 2.78 (br t, J=5.48 Hz, 2H) 3.78 (s, 3H) 4.34 (br s, 2H) 4.77-4.91 (m, 4H) 5.74 (br s, 2H) 7.24-7.48 (m, 4H) 7.65 (d, J=4.29 Hz, 2H) 7.99 (br d, J=5.60 Hz, 2H) 9.62 (s, 1H). LCMS (ESI+): m/z 753.1 (M+1)+, RT: 1.192 min.

Synthesis of tert-butyl (E)-3-(1-(3-((5-carbamoyl-1-(4-(5-carbamoyl-2-(2,4-dimethyloxazole-5-carboxamido)-7-methoxy-1H-benzo[d]imidazol-1-yl)but-2-en-1-yl)-2-(2,4-dimethyloxazole-5-carboxamido)-1H-benzo[d]imidazol-7-yl)oxy)propyl)piperidin-4-yl)-5,6-dihydro-[1,2,4]triazolo[4,3-a]pyrazine-7 (8H)-carboxylate (I-43-7): Compound I-43-6 (200 mg, 239.13 μmol, 1 eq) was dissolved in N,N-dimethylformamide (4 mL), tert-butyl 3-(piperidin-4-yl)-5,6-dihydro-[1,2,4]triazolo[4,3-a]pyrazine-7 (8H)-carboxylate (88.21 mg, 286.96 μmol, 1.2 eq), and NaBH(OAc)3 (70.95 mg, 334.78 μmol, 1.5 eq) were serially added. The reaction system was stirred at 25° C. for 2 hrs. The residue was diluted with dimethylsulfoxide (10 mL) and purified by prep-HPLC (TFA condition) to give tert-butyl (E)-3-(1-(3-((5-carbamoyl-1-(4-(5-carbamoyl-2-(2,4-dimethyloxazole-5-carboxamido)-7-methoxy-1Hbenzo[d]imidazol-1-yl)but-2-en-1-yl)-2-(2,4-dimethyloxazole-5-carboxamido)-1Hbenzo[d]imidazol-7-yl)oxy)propyl)piperidin-4-yl)-5,6-dihydro-[1,2,4]triazolo[4,3-a]pyrazine-7 (8H)-carboxylate (58 mg, 21% yield, 89% purity) as white solid. LCMS (ESI+): m/z 522.7 (1/2M+1)+, RT: 1.272 min.

Synthesis of (E)-N-(5-carbamoyl-1-(4-(5-carbamoyl-2-(2,4-dimethyloxazole-5-carboxamido)-7-(3-(4-(5,6,7,8-tetrahydro-[1,2,4]triazolo[4,3-a]pyrazin-3-yl)piperidin-1-yl)propoxy)-1H-benzo[d]imidazol-1-yl)but-2-en-1-yl)-7-methoxy-1H-benzo[d]imidazol-2-yl)-2,4-dimethyloxazole-5-carboxamide (I-43): Compound I-43-7 (50 mg, 47.89 μmol, 1 eq) was dissolved in dichloromethane (0.4 mL), trifluoroacetic acid (0.1 mL) was added dropwise. The reaction system was stirred at 25° C. for 2 hs and concentrated under reduced pressure. The residue was purified by prep-HPLC (TFA condition) to obtain Compound 43 (15 mg, 33% yield, 100% purity) as white solid. 1H NMR (400 MHz, DMSO-d6) δ ppm 1.87-2.01 (m, 4H) 2.06 (br d, J=11.92 Hz, 2H) 2.24-2.31 (m, 3H) 2.31-2.43 (m, 9H) 2.78-2.94 (m, 2H) 2.98-3.15 (m, 3H) 3.16 (br s, 2H) 3.65 (br d, J=4.29 Hz, 3H) 3.75 (s, 3H) 3.99-4.08 (m, 2H) 4.13-4.22 (m, 2H) 4.55 (s, 2H) 4.83-4.96 (m, 4H) 5.61-5.81 (m, 2H) 7.29 (s, 1H) 7.33 (s, 1H) 7.38 (br s, 2H) 7.66 (d, J=4.77 Hz, 2H) 7.86-8.08 (m, 2H) 9.31-9.61 (m, 2H) 12.61-12.93 (m, 2H). LCMS (ESI+): m/z 472.8 (1/2M+1)+, RT: 1.774 min.

### Synthesis 2AA. Compound 61

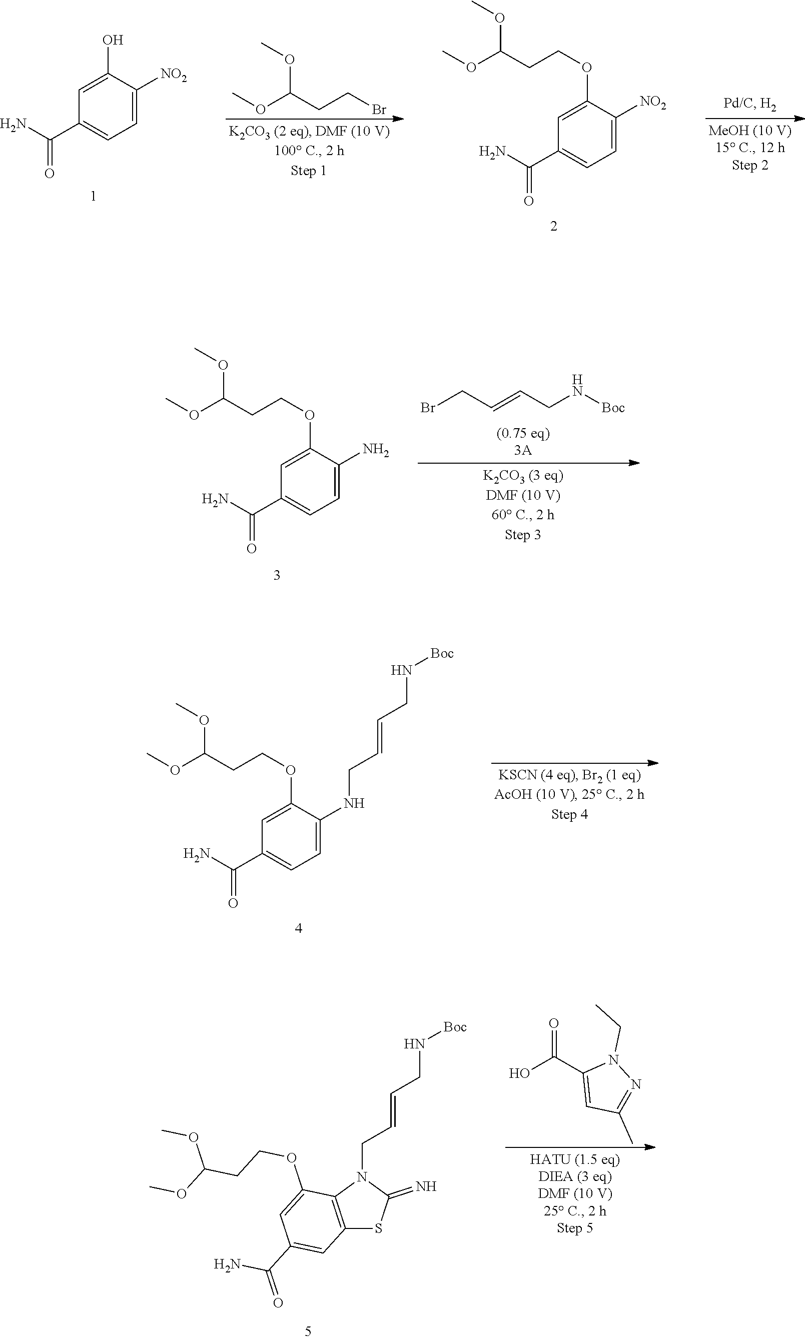

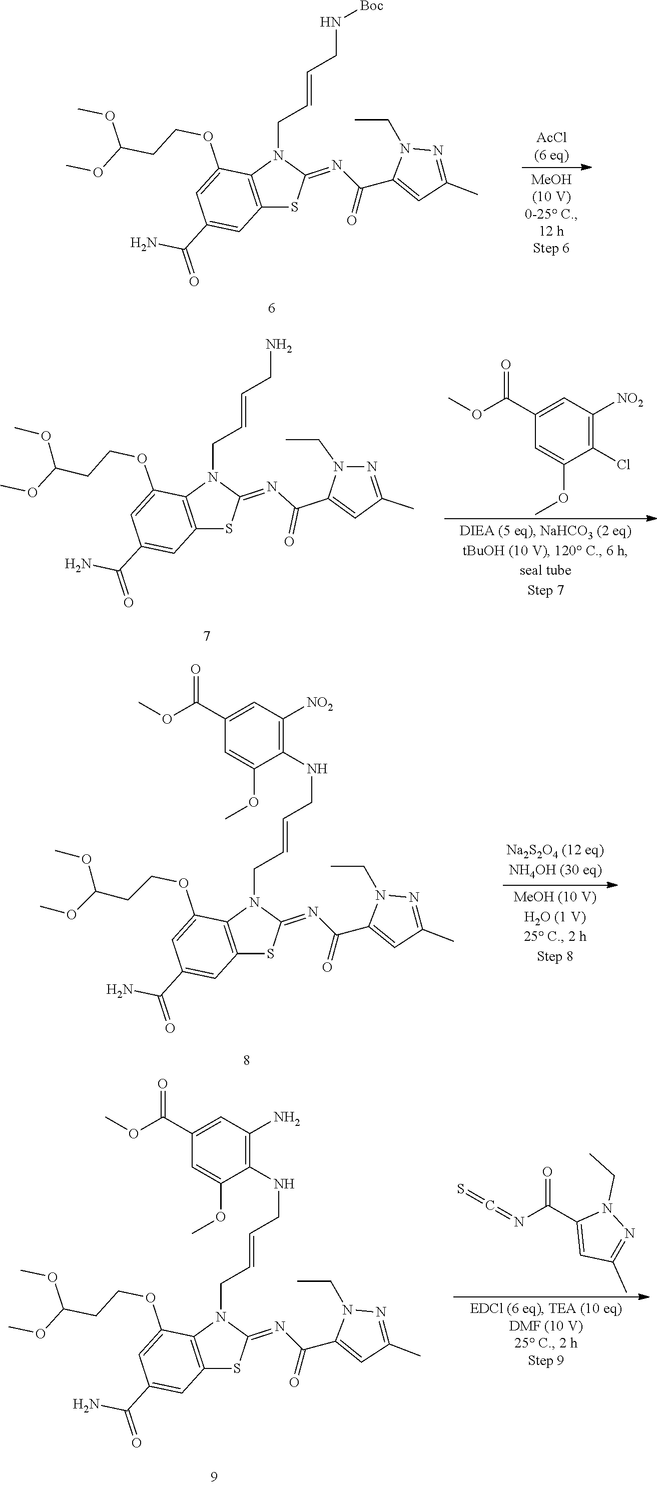

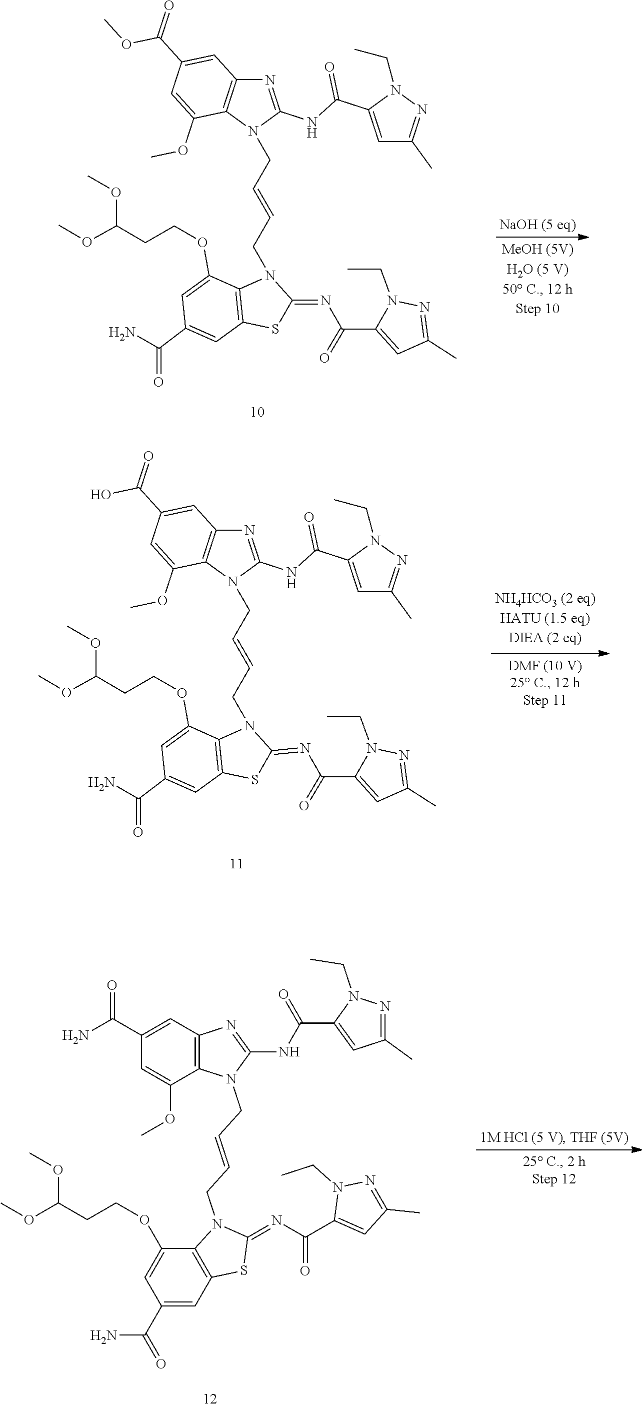

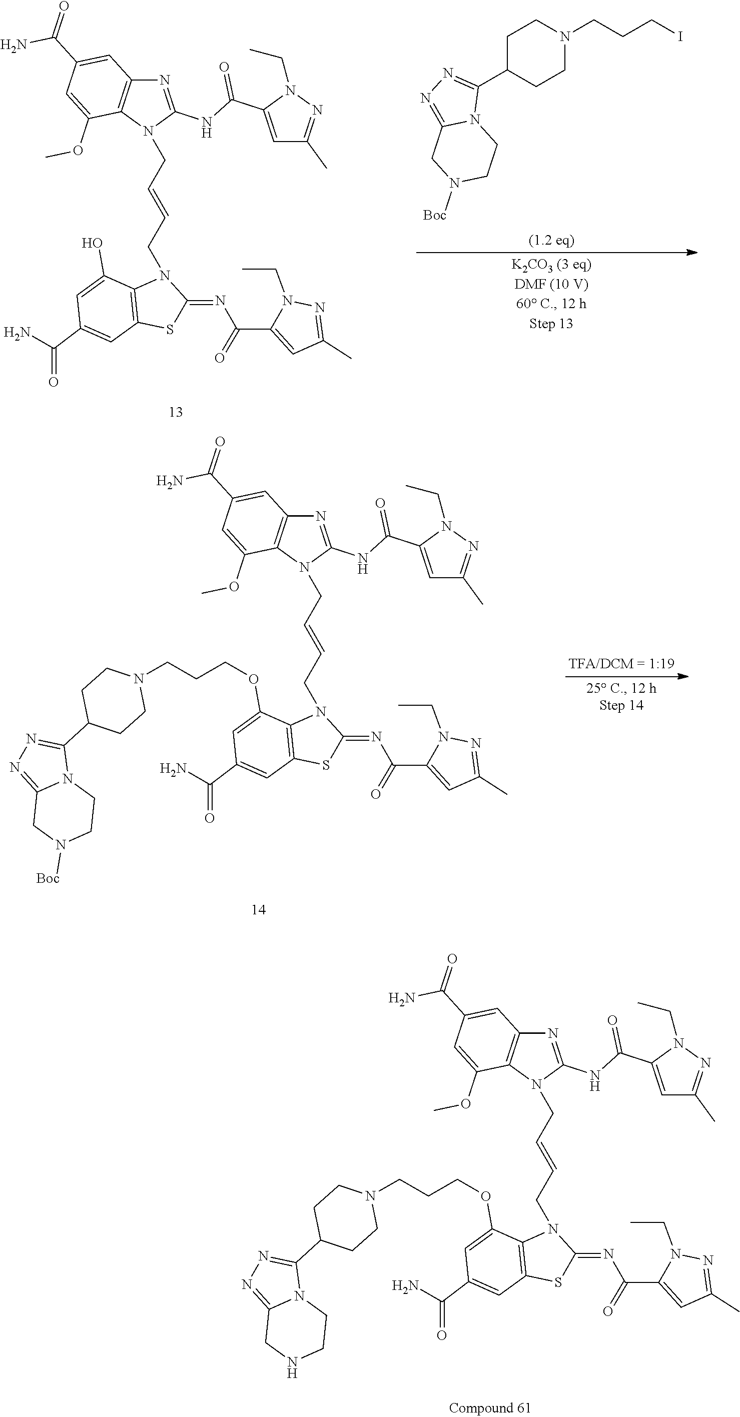 [SMILES: CCn1nc(C)cc1C(=O)/N=c1\sc2cc(C(N)=O)cc(O)c2n1C/C=C/Cn1c(NC(=O)c2cc(C)nn2CC)nc2cc(C(N)=O)cc(OC)c21 ; CC(C)(C)OC(=O)N1CCn2c(nnc2C2CCN(CCCI)CC2)C1 ; CCn1nc(C)cc1C(=O)/N=c1\sc2cc(C(N)=O)cc(OCCCN3CCC(c4nnc5n4CCN(C(=O)OC(C)(C)C)C5)CC3)c2n1C/C=C/Cn1c(NC(=O)c2cc(C)nn2CC)nc2cc(C(N)=O)cc(OC)c21 ; CCn1nc(C)cc1C(=O)/N=c1\sc2cc(C(N)=O)cc(OCCCN3CCC(c4nnc5n4CCNC5)CC3)c2n1C/C=C/Cn1c(NC(=O)c2cc(C)nn2CC)nc2cc(C(N)=O)cc(OC)c21]

Synthesis of 3-(3,3-dimethoxypropoxy)-4-nitrobenzamide (AA2): Compound AA1 (10 g, 54.90 mmol, 1 eq) and 3-bromo-1,1-dimethoxy-propane (12.06 g, 65.89 mmol, 1.2 eq) were dissolved in N,N-dimethylformamide (100 mL). And potassium carbonate (15.18 g, 109.81 mmol, 2 eq) was added to the mixture. The reaction system was heated to 100° C. and stirred for 2 hrs. After that the reaction was quenched by addition water (50 mL) at 25° C. and extracted with ethyl acetate (100 mL×3). The combined organic layers were washed with brine (100 mL×3), dried over Na2SO4, filtered, and concentrated under reduced pressure. The residue was purified by re-crystallization from petroleum ether/ethyl acetate (v/v)=10/1 (100 mL) at 15° C. to give 3-(3,3-dimethoxypropoxy)-4-nitrobenzamide (14.9 g, 89% yield, 93% purity) as white solid. 1H NMR (400 MHz, DMSO-d6) δ ppm 2.01 (q, J=6.16 Hz, 2H) 3.25 (s, 6H) 4.24 (t, J=6.20 Hz, 2H) 4.57 (t, J=5.66 Hz, 1H) 7.57 (dd, J=8.34, 1.43 Hz, 1H) 7.66-7.79 (m, 2H) 7.94 (d, J=8.34 Hz, 1H) 8.24 (br s, 1H). LCMS (ESI+): m/z 306.9 (M+Na)+, RT: 1.152 min.

Synthesis of 4-amino-3-(3,3-dimethoxypropoxy)benzamide (AA3): Compound AA2 (13.9 g, 93% purity, 45.47 mmol, 1 eq) was dissolved in methanol (140 mL). Pd/C (10%, 7 g) was added to the mixture under N2 atmosphere. The suspension was degassed and purged with H2 for three times. The mixture was stirred under H2 (15 Psi) at 15° C. for 12 hrs. Then the reaction was filtered, the filtrate was concentrated under reduced pressure to get 4-amino-3-(3,3-dimethoxypropoxy)benzamide (12 g, 89% purity, 86% yield) as colorless oil which was used in the next step directly without further purification. 1H NMR (400 MHz, DMSO-d6) δ ppm 2.01 (q, J=6.24 Hz, 2H) 3.27 (s, 6H) 3.99 (t, J=6.30 Hz, 2H) 4.68 (t, J=5.81 Hz, 1H) 5.20-5.29 (m, 2H) 6.59 (d, J=7.83 Hz, 1H) 6.75-7.01 (m, 1H) 7.21-7.40 (m, 2H) 7.59 (br s, 1H). LCMS (ESI+): m/z 277.1 (M+Na)+, RT: 0.721 min.

Synthesis of tert-butyl (E)-(4-((4-carbamoyl-2-(3,3-dimethoxypropoxy)phenyl)amino)but-2-en-1-yl)carbamate (AA4): Compound AA3 (5 g, 89% purity, 17.50 mmol, 1 eq) was dissolved in N,N-dimethylformamide (50 mL). And tert-butyl (E)-(4-bromobut-2-en-1-yl)carbamate (3.28 g, 13.12 mmol, 0.75 eq) and potassium carbonate (7.26 g, 52.50 mmol, 3 eq) were serially added to the mixture. The reaction system was heated to 60° C. and stirred for 2 hrs. After cooling to room temperature, the reaction was quenched by addition water (50 mL) and extracted with ethyl acetate (50 mL×3). The combined organic layers were washed with brine (50 mL×3), dried over Na2SO4, filtered, and concentrated under reduced pressure. The residue was purified by prep-HPLC (neutral condition) to give tert-butyl (E)-(4-((4-carbamoyl-2-(3,3-dimethoxypropoxy)phenyl)amino)but-2-en-1-yl)carbamate (3.8 g, 98% purity, 45% yield) as white solid. 1H NMR (400 MHz, DMSO-d6) δ ppm 1.31-1.40 (m, 9H) 2.03 (q, J=6.16 Hz, 2H) 3.27 (s, 6H) 3.51 (br s, 2H) 3.76 (br d, J=4.65 Hz, 2H) 4.01 (t, J=6.32 Hz, 2H) 4.68 (t, J=5.72 Hz, 1H) 5.56 (s, 3H) 6.46 (d, J=8.34 Hz, 1H) 6.80-7.00 (m, 2H) 7.30 (d, J=1.67 Hz, 1H) 7.37 (dd, J=8.29, 1.49 Hz, 1H) 7.61 (br s, 1H). LCMS (ESI+): m/z 424.2 (M+1)+, RT: 1.098 min.

Synthesis of tert-butyl (E)-(4-(6-carbamoyl-4-(3,3-dimethoxypropoxy)-2-iminobenzo[d]thiazol-3 (2H)-yl)but-2-en-1-yl)carbamate (AA5): Compound AA4 (1.6 g, 98% purity, 3.70 mmol, 1 eq) was dissolved in acetic acid (10 mL). And potassium thiocyanate (1.44 g, 14.81 mmol, 4 eq) was serially added. The reaction system was stirred at 25° C. for 0.5 hr. Then, liquid bromine (591.67 mg, 3.70 mmol, 1 eq) dissolved in acetic acid (6 mL) was added dropwise, and the resulting mixture was stirred at 25° C. for 1.5 hrs. After the reaction was completed, the mixture was adjusted to pH 9 with aqueous ammonia and extracted with ethyl acetate (30 mL×3) and washed with saturated brine (30 mL×3). The organic phase was dried over anhydrous sodium sulfate and filtered, and the filtrate was concentrated under reduced pressure to give tert-butyl (E)-(4-(6-carbamoyl-4-(3,3-dimethoxypropoxy)-2-iminobenzo[d]thiazol-3 (2H)-yl)but-2-en-1-yl)carbamate (1.8 g, 68% purity, 69% yield) as off-white solid. 1H NMR (400 MHz, DMSO-d6) δ ppm 1.27-1.39 (m, 9H) 2.01-2.13 (m, 2H) 3.28 (s, 5H) 3.38-3.63 (m, 2H) 4.08-4.22 (m, 2H) 4.58 (t, J=5.50 Hz, 1H) 4.86 (br d, J=2.69 Hz, 2H) 5.37-5.75 (m, 2H) 6.78-7.04 (m, 1H) 7.22-7.50 (m, 2H) 7.52-7.72 (m, 1H) 7.85-7.99 (m, 1H) 8.56-8.64 (m, 1H). LCMS (ESI+): m/z 481.1 (M+1)+, RT: 1.018 min.

Synthesis of tert-butyl ((E)-4-((Z)-6-carbamoyl-4-(3,3-dimethoxypropoxy)-2-((1-ethyl-3-methyl-1H-pyrazole-5-carbonyl)imino)benzo[d]thiazol-3 (2H)-yl)but-2-en-1-yl)carbamate (AA6): Compound AA5 (1.8 g, 68% purity, 2.54 mmol, 1 eq) was dissolved in dimethyl formamide (18 mL). 1-Ethyl-3-methyl-1H-pyrazole-5-carboxylic acid (470.49 mg, 3.05 mmol, 1.2 eq), 0-(7-azabenzotriazol-1-yl)-N,N,N,N-tetramethyluronium hexafluorophosphate (1.45 g, 3.81 mmol, 1.5 eq) and diisopropylethylamine (986.09 mg, 7.63 mmol, 3 eq) were serially added to the mixture. The reaction system was stirred at 25° C. for 2 hrs and then diluted with water (10 mL) and extracted with ethyl acetate (20 mL×3). The organic phases were combined, dried over anhydrous sodium sulfate, and filtered, and the filtrate was concentrated under reduced pressure. The solid crude product was purified by silica gel column chromatography (petroleum ether/ethyl acetate (v/v)=1/0-0/1) to obtain tert-butyl ((E)-4-((Z)-6-carbamoyl-4-(3,3-dimethoxypropoxy)-2-((1-ethyl-3-methyl-1H-pyrazole-5-carbonyl)imino)benzo[d]thiazol-3 (2H)-yl)but-2-en-1-yl)carbamate (2.2 g, 83% purity, 80% yield) as off-white solid. 1H NMR (400 MHz, DMSO-d6) δ ppm 1.37 (br s, 12H) 2.14 (q, J=6.24 Hz, 2H) 2.20 (s, 3H) 3.31 (s, 5H) 3.51 (br s, 2H) 4.24 (br t, J=6.36 Hz, 2H) 4.54-4.67 (m, 3H) 5.38 (br d, J=4.40 Hz, 2H) 5.53-5.85 (m, 2H) 6.78 (s, 1H) 6.91 (br t, J=5.38 Hz, 1H) 7.49 (br s, 1H) 7.61 (s, 1H) 8.00 (d, J=1.10 Hz, 1H) 8.12 (br s, 1H). LCMS (ESI+): m/z 617.3 (M+1)+, RT: 1.257 min.

Synthesis of (Z)-3-((E)-4-aminobut-2-en-1-yl)-4-(3,3-dimethoxypropoxy)-2-((1-ethyl-3-methyl-1H-pyrazole-5-carbonyl)imino)-2,3-dihydrobenzo[d]thiazole-6-carboxamide (AA7): Acetyl chloride (1.39 g, 17.76 mmol, 6 eq) was dissolved in methanol (22 mL) at 0° C. The mixture was stirred for 1 hr at 0° C. Then compound AA6 (2.2 g, 2.96 mmol, 83% purity, 1 eq) was added to the mixture and the reaction was stirred at 25° C. for 11 hrs. The reaction was quenched by adding NaHCO3 aqueous solution (10 mL) and extracted with ethyl acetate (20 mL×3) and washed with saturated brine (10 mL×3). The organic phase was dried over anhydrous sodium sulfate and filtered, and the filtrate was concentrated under reduced pressure to give (Z)-3-((E)-4-aminobut-2-en-1-yl)-4-(3,3-dimethoxypropoxy)-2-((1-ethyl-3-methyl-1H-pyrazole-5-carbonyl)imino)-2,3-dihydrobenzo[d]thiazole-6-carboxamide (1.5 g, 84% purity, 82% yield) as off-white solid. 1H NMR (400 MHz, DMSO-d6) δ ppm 1.04 (d, J=5.99 Hz, 1H) 1.29-1.40 (m, 3H) 2.10-2.24 (m, 5H) 3.25-3.34 (m, 8H) 3.59-3.76 (m, 1H) 4.25 (br t, J=6.11 Hz, 2H) 4.48-4.74 (m, 3H) 5.42 (br d, J=4.65 Hz, 2H) 5.65 (dt, J=15.22, 5.72 Hz, 1H) 5.88-6.08 (m, 1H) 6.70-6.91 (m, 1H) 7.41-7.58 (m, 1H) 7.58-7.67 (m, 1H) 7.99-8.08 (m, 1H) 8.09-8.20 (m, 1H). LCMS (ESI+): m/z 517.1 (M+1)+, RT: 0.981 min.

Synthesis of methyl 4-(((E)-4-((Z)-6-carbamoyl-4-(3,3-dimethoxypropoxy)-2-((1-ethyl-3-methyl-1H-pyrazole-5-carbonyl)imino)benzo [d]thiazol-3 (2H)-yl)but-2-en-1-yl)amino)-3-methoxy-5-nitrobenzoate (AA8): Compound AA7 (700 mg, 84% purity, 1.14 mmol, 1 eq) was dissolved in t-butanol (7 mL). Methyl 4-chloro-3-methoxy-5-nitrobenzoate (335.46 mg, 1.37 mmol, 1.2 eq), diisopropylethylamine (735.53 mg, 5.69 mmol, 5 eq) and sodium bicarbonate (191.23 mg, 2.28 mmol, 2 eq) were serially added to the mixture. The reaction system was heated to 120° C. in a sealed tube and stirred for 6 hrs and then poured into water. The resulting solid was filtered and purified by silica gel column chromatography (ethyl acetate/methanol (v/v)=1/0-10/1) to obtain methyl 4-(((E)-4-((Z)-6-carbamoyl-4-(3,3-dimethoxypropoxy)-2-((1-ethyl-3-methyl-1H-pyrazole-5-carbonyl)imino)benzo[d]thiazol-3 (2H)-yl)but-2-en-1-yl)amino)-3-methoxy-5-nitrobenzoate (800 mg, 78% purity, 75% yield) as orange solid. LCMS (ESI+): m/z 726.0 (M+1)+, RT: 1.316 min.

Synthesis of methyl 3-amino-4-(((E)-4-((Z)-6-carbamoyl-4-(3,3-dimethoxypropoxy)-2-((1-ethyl-3-methyl-1H-pyrazole-5-carbonyl)imino)benzo[d]thiazol-3 (2H)-yl)but-2-en-1-yl)amino)-5-methoxybenzoate (AA9): Compound AA8 (800 mg, 78% purity, 855.36 μmol, 1 eq) was dissolved in methanol (8 mL). An aqueous solution (0.8 mL) of sodium dithionite (1.79 g, 10.26 mmol, 12 eq) and aqueous ammonia (899.31 mg, 25.66 mmol, 1.90 mL, 27% aqueous solution, 30 eq) were serially added to the mixture. The reaction system was stirred at 25° C. for 2 hrs. After the reaction was completed, the mixture was diluted with methanol (10 mL) and ethyl acetate (100 mL) and washed with saturated brine (30 mL×3). The organic phase was dried over anhydrous sodium sulfate and filtered, and the filtrate was concentrated under reduced pressure to give methyl 3-amino-4-(((E)-4-((Z)-6-carbamoyl-4-(3,3-dimethoxypropoxy)-2-((1-ethyl-3-methyl-1H-pyrazole-5-carbonyl)imino)benzo[d]thiazol-3 (2H)-yl)but-2-en-1-yl)amino)-5-methoxybenzoate (700 mg, 84% purity, 77% yield) as yellow solid. LCMS (ESI+): m/z 696.3 (M+1)+, RT: 1.199 min.

Synthesis of methyl 1-((E)-4-((Z)-6-carbamoyl-4-(3,3-dimethoxypropoxy)-2-((1-ethyl-3-methyl-1H-pyrazole-5-carbonyl)imino)benzo [d]thiazol-3 (2H)-yl)but-2-en-1-yl)-2-(1-ethyl-3-methyl-1H-pyrazole-5-carboxamido)-7-methoxy-1H-benzo[d]imidazole-5-carboxylate (AA10): To a solution of Compound AA9C (700 mg, 84% purity, 845.08 μmol, 1 eq) in N, N-dimethylformamide (7 mL) was added compound AA9A (329.99 mg, 1.69 mmol, 2 eq), 1-ethyl-(3-dimethylaminopropyl)carbonyldiimine hydrochloride (972.03 mg, 5.07 mmol, 6 eq) and triethylamine (855.16 mg, 8.45 mmol, 10 eq). The reaction system was stirred at 25° C. for 2 hrs. The reaction was quenched by adding water (20 mL), and the resulting solid was filtered, washed with water (5 mL) and dried to afford methyl 1-((E)-4-((Z)-6-carbamoyl-4-(3,3-dimethoxypropoxy)-2-((1-ethyl-3-methyl-1H-pyrazole-5-carbonyl)imino)benzo[d]thiazol-3 (2H)-yl)but-2-en-1-yl)-2-(1-ethyl-3-methyl-1H-pyrazole-5-carboxamido)-7-methoxy-1H-benzo[d]imidazole-5-carboxylate (700 mg, 84% purity, 82% yield) as white solid. LCMS (ESI+): m/z 857.2 (M+1)+, RT: 1.329 min.

Synthesis of 1-((E)-4-((Z)-6-carbamoyl-4-(3,3-dimethoxypropoxy)-2-((1-ethyl-3-methyl-1H-pyrazole-5-carbonyl)imino)benzo[d]thiazol-3 (2H)-yl)but-2-en-1-yl)-2-(1-ethyl-3-methyl-1H-pyrazole-5-carboxamido)-7-methoxy-1H-benzo[d]imidazole-5-carboxylic acid (AA11): Compound AA10 (700 mg, 84% purity, 689.42 μmol, 1 eq) was dissolved in methanol (3.5 mL). An aqueous solution (3.5 mL) of sodium hydroxide (137.87 mg, 3.45 mmol, 5 eq) was added dropwise to the mixture. The reaction system was stirred at 50° C. for 12 hrs. The reaction was adjusted to pH=7 with 1 M hydrochloric acid aqueous solution and extracted with isopropanol/chloroform (v/v)=1/3, 30 mL×3). The combined organic layers were dried over Na2SO4 and concentrated under reduced pressure to give 1-((E)-4-((Z)-6-carbamoyl-4-(3,3-dimethoxypropoxy)-2-((1-ethyl-3-methyl-1H-pyrazole-5-carbonyl)imino)benzo[d]thiazol-3 (2H)-yl)but-2-en-1-yl)-2-(1-ethyl-3-methyl-1H-pyrazole-5-carboxamido)-7-methoxy-1H-benzo[d]imidazole-5-carboxylic acid (500 mg, 74% purity, 64% yield) as white solid. LCMS (ESI+): m/z: 843.3 (M+1)+, RT: 1.021 min.

Synthesis of (Z)-3-((E)-4-(5-carbamoyl-2-(1-ethyl-3-methyl-1H-pyrazole-5-carboxamido)-7-methoxy-1H-benzo[d]imidazol-1-yl)but-2-en-1-yl)-4-(3,3-dimethoxypropoxy)-2-((1-ethyl-3-methyl-1H-pyrazole-5-carbonyl)imino)-2,3-dihydrobenzo[d]thiazole-6-carboxamide (AA12): Compound AA11 (500 mg, 74% purity, 438.35 μmol, 1 eq) was dissolved in dimethyl formamide (5 mL). NH4HCO3 (69.31 mg, 876.70 μmol, 2 eq), 0-(7-azabenzotriazol-1-yl)-N,N,N,N-tetramethyluronium hexafluorophosphate (250.02 mg, 657.53 μmol, 1.5 eq) and diisopropylethylamine (113.31 mg, 876.70 μmol, 2 eq) were serially added to the mixture. The reaction system was stirred at 25° C. for 12 hrs. The reaction was quenched by adding water (20 mL), and the resulting solid was filtered, washed with water (5 mL) and dried to afford (Z)-3-((E)-4-(5-carbamoyl-2-(1-ethyl-3-methyl-1H-pyrazole-5-carboxamido)-7-methoxy-1H-benzo[d]imidazol-1-yl)but-2-en-1-yl)-4-(3,3-dimethoxypropoxy)-2-((1-ethyl-3-methyl-1H-pyrazole-5-carbonyl)imino)-2,3-dihydrobenzo[d]thiazole-6-carboxamide (400 mg, 90% purity, 97% yield) as white solid. LCMS (ESI+): m/z: 842.3 (M+1)+, RT: 1.148 min.

Synthesis of (Z)-3-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-((1-ethyl-3-methyl-1H-pyrazole-5-carbonyl)imino)-4-hydroxy-2,3-dihydrobenzo[d]thiazole-6-carboxamide (AA13): Compound AA12 (400 mg, 90% purity, 425.68 μmol, 1 eq) was dissolved in tetrahydrofuran (2 mL). 1 M hydrochloric acid aqueous solution (2 mL) was added to the mixture dropwise. The reaction system was stirred at 25° C. for 2 hrs. The reaction was adjusted to pH=7 with sodium bicarbonate aqueous solution, and the resulting solid was filtered, washed with water (10 mL) and dried to afford (Z)-3-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-((1-ethyl-3-methyl-1H-pyrazole-5-carbonyl)imino)-4-hydroxy-2,3-dihydrobenzo[d]thiazole-6-carboxamide (200 mg, 90% purity, 57% yield) as white solid. LCMS (ESI+): m/z: 740.1 (M+1)+, RT: 1.070 min.

Synthesis of tert-butyl 3-(1-(3-(((Z)-6-carbamoyl-3-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-((1-ethyl-3-methyl-1H-pyrazole-5-carbonyl)imino)-2,3-dihydrobenzo[d]thiazol-4-yl)oxy)propyl)piperidin-4-yl)-5,6-dihydro-[1,2,4]triazolo[4,3-a]pyrazine-7 (8H)-carboxylate (AA14): Compound AA13 (200 mg, 90% purity, 243.3 μmol, 1 eq) was dissolved in dimethyl formamide (2 mL). Then tert-butyl 3-(1-(3-iodopropyl)piperidin-4-yl)-5,6-dihydro-[1,2,4]triazolo[4,3-a]pyrazine-7 (8H)-carboxylate (138.79 mg, 291.9 μmol, 1.2 eq), and potassium carbonate (100.88 mg, 729.9 μmol, 3 eq) were serially added to the mixture. The reaction system was heated to 60° C. and stirred for 12 hrs. After the reaction was completed, the mixture was diluted with ethyl acetate (10 mL×3) and washed with saturated brine (10 mL×3). The organic phase was dried over anhydrous sodium sulfate and filtered, and the filtrate was concentrated under reduced pressure. The residue was purified by prep-HPLC (TFA condition) to give tert-butyl 3-(1-(3-(((Z)-6-carbamoyl-3-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-((1-ethyl-3-methyl-1H-pyrazole-5-carbonyl)imino)-2,3-dihydrobenzo[d]thiazol-4-yl)oxy)propyl)piperidin-4-yl)-5,6-dihydro-[1,2,4]triazolo[4,3-a]pyrazine-7 (8H)-carboxylate (157 mg, 95% purity, 56% yield) as white solid. LCMS (ESI+): m/z 544.5 (1/2M+1)+, RT: 1.195 min.

Synthesis of (Z)-3-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-((1-ethyl-3-methyl-1H-pyrazole-5-carbonyl)imino)-4-(3-(4-(5,6,7,8-tetrahydro-[1,2,4]triazolo[4,3-a]pyrazin-3-yl)piperidin-1-yl)propoxy)-2,3-dihydrobenzo[d]thiazole-6-carboxamide (61): To a solution of compound AA14 (157 mg, 95% purity, 137.18 μmol, 1 eq) in dichloromethane (3 mL) was added trifluoroacetic acid (0.15 mL) dropwise. The reaction system was stirred at 25° C. for 12 hrs and concentrated under reduced pressure. The residue was purified by prep-HPLC (TFA condition) to obtain (Z)-3-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-((1-ethyl-3-methyl-1H-pyrazole-5-carbonyl)imino)-4-(3-(4-(5,6,7,8-tetrahydro-[1,2,4]triazolo[4,3-a]pyrazin-3-yl)piperidin-1-yl)propoxy)-2,3-dihydrobenzo[d]thiazole-6-carboxamide (106 mg, 76% yield, 97% purity) as white solid. 1H NMR (400 MHz, DMSO-d6) δ ppm 1.18-1.38 (m, 6H) 1.87-2.06 (m, 5H) 2.06-2.20 (m, 7H) 2.81-3.07 (m, 3H) 3.12 (br s, 2H) 3.21-3.34 (m, 1H) 3.48 (brd, J=11.88 Hz, 2H) 3.63-3.70 (m, 2H) 3.74 (s, 3H)4.04-4.09 (m, 2H) 4.18-4.21 (m, 2H) 4.50-4.57 (m, 5H) 4.89 (br d, J=3.25 Hz, 2H) 5.35 (br s, 2H) 5.67-5.89 (m, 2H) 6.42-6.56 (m, 1H) 6.62 (s, 1H) 7.25-7.43 (m, 2H) 7.46-7.57 (m, 2H) 7.60-7.69 (m, 1H) 7.92-8.17 (m, 3H) 9.49-9.72 (m, 2H) 12.56-13.12 (m, 1H). LCMS (ESI+): m/z 987.4 (M+1)+, RT: 1.973 min.

### Synthesis 2AB. Compound 86

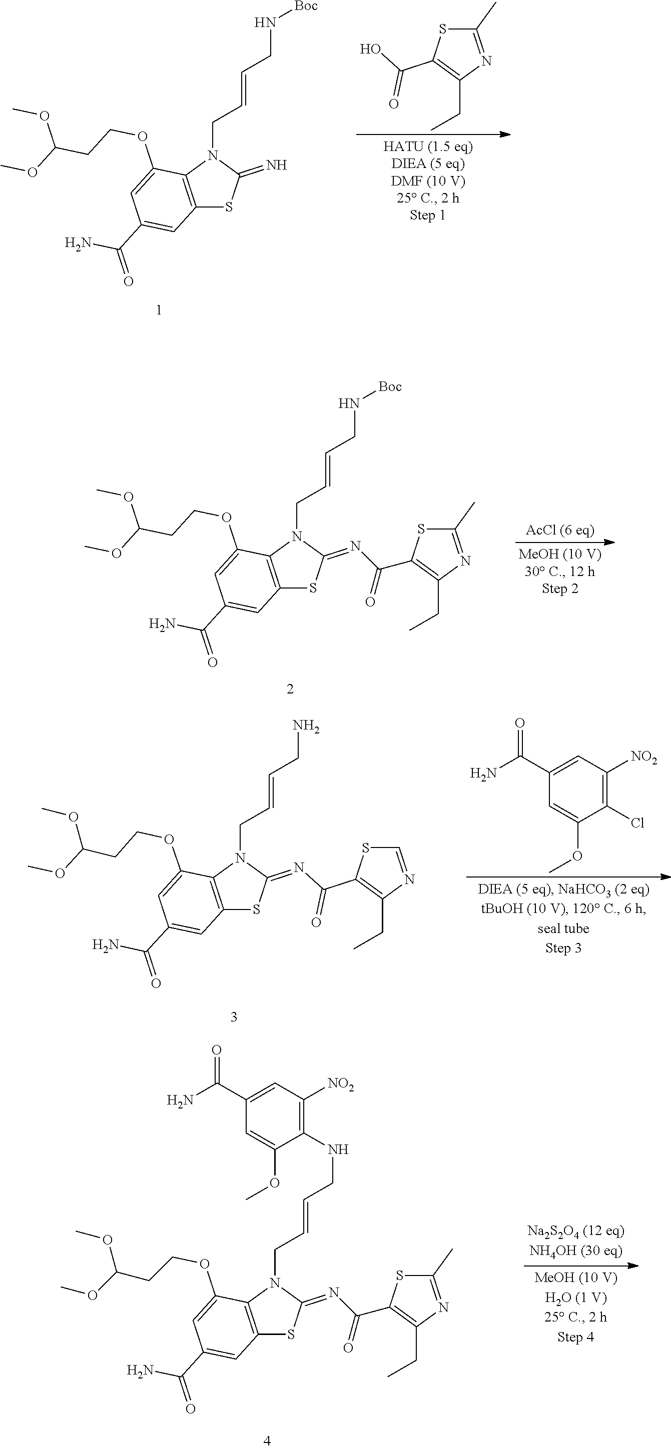

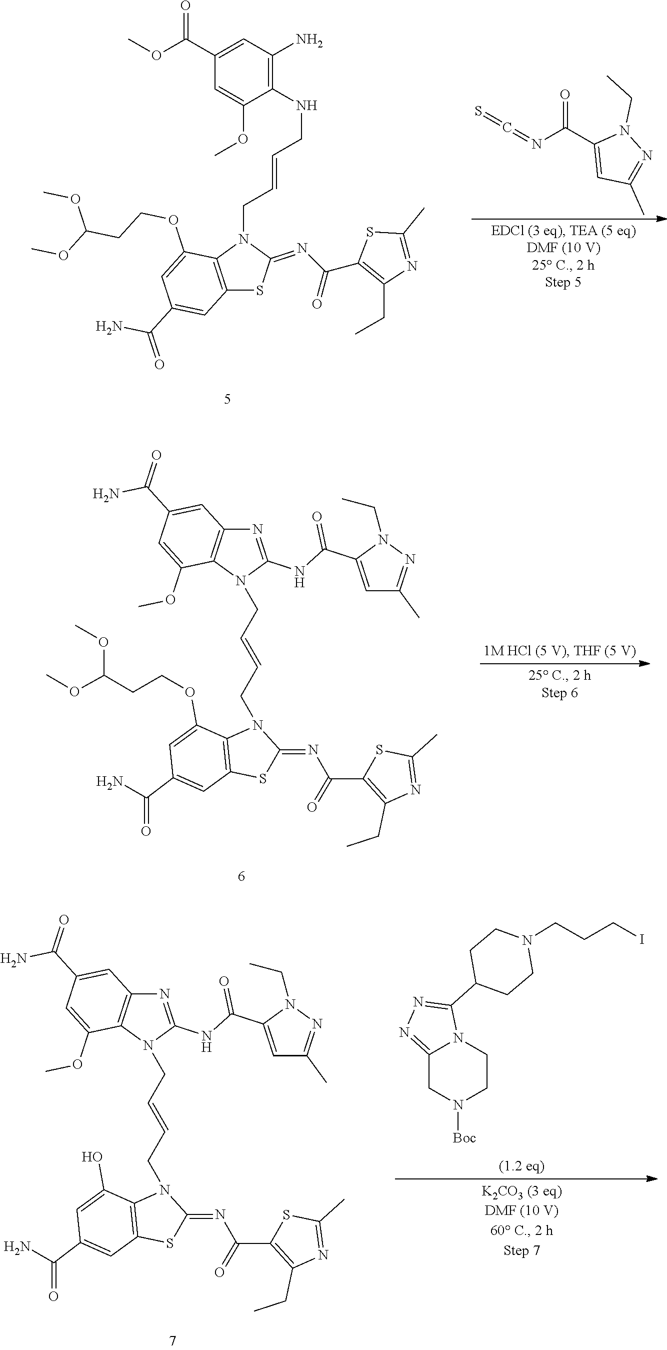

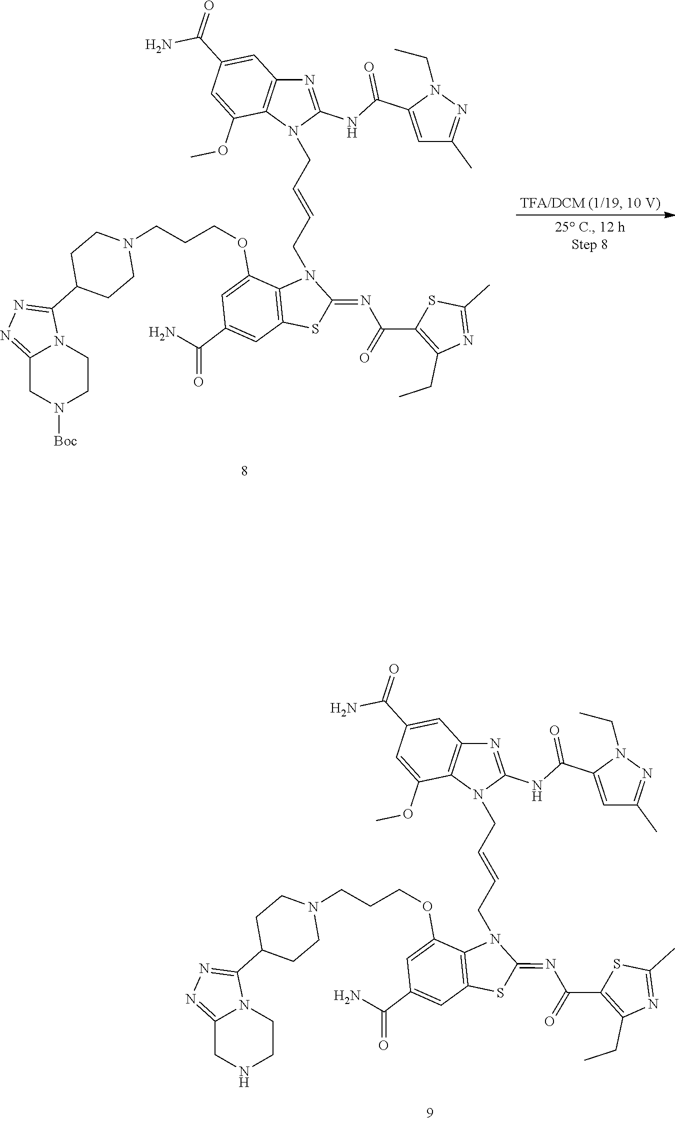 [SMILES: CCc1nc(C)sc1C(=O)/N=c1\sc2cc(C(N)=O)cc(OCCCN3CCC(c4nnc5n4CCN(C(=O)OC(C)(C)C)C5)CC3)c2n1C/C=C/Cn1c(NC(=O)c2cc(C)nn2CC)nc2cc(C(N)=O)cc(OC)c21 ; CCc1nc(C)sc1C(=O)/N=c1\sc2cc(C(N)=O)cc(OCCCN3CCC(c4nnc5n4CCNC5)CC3)c2n1C/C=C/Cn1c(NC(=O)c2cc(C)nn2CC)nc2cc(C(N)=O)cc(OC)c21]

Synthesis of tert-butyl N—[(E)-4-[(2Z)-6-carbamoyl-4-(3,3-dimethoxypropoxy)-2-(4-ethyl-2-methyl-thiazole-5-carbonyl)imino-1,3-benzothiazol-3-yl]but-2-enyl]carbamate (AB2): Compound AB1 (3 g, 81% purity, 5.05 mmol, 1 eq) was dissolved in dimethyl formamide (30 mL). 4-Ethyl-2-methyl-thiazole-5-carboxylic acid (1.04 g, 6.06 mmol, 1.2 eq), 0-(7-azabenzotriazol-1-yl)-N,N,N,N-tetramethyluronium hexafluorophosphate (2.88 g, 7.58 mmol, 1.5 eq) and diisopropylethylamine (3.26 g, 25.25 mmol, 4.40 mL, 5 eq) were serially added to the mixture. The reaction system was stirred at 25° C. for 2 hrs and then diluted with water (10 mL) and extracted with ethyl acetate (20 mL×3). The organic phases were combined, dried over anhydrous sodium sulfate, and filtered, and the filtrate was concentrated under reduced pressure. The solid crude product was purified by silica gel column chromatography (petroleum ether/ethyl acetate (v/v)=1/0-0/1) to obtain tert-butyl N—[(E)-4-[(2Z)-6-carbamoyl-4-(3,3-dimethoxypropoxy)-2-(4-ethyl-2-methyl-thiazole-5-carbonyl)imino-1,3-benzothiazol-3-yl]but-2-enyl]carbamate, 2 (3.2 g, 91% purity, 91% yield) as off-white solid. LCMS (ESI+): m/z 634.3 (M+1)+, RT: 1.297 min.

Synthesis of (Z)-3-((E)-4-aminobut-2-en-1-yl)-4-(3,3-dimethoxypropoxy)-2-((4-ethyl-2-methylthiazole-5-carbonyl)imino)-2,3-dihydrobenzo[d]thiazole-6-carboxamide (AB3): Acetyl chloride (2.16 g, 27.51 mmol, 1.96 mL, 6 eq) was dissolved in methanol (32 mL) at 0° C. and stirred for 1 hr. Then compound AB2 (3.2 g, 4.58 mmol, 91% purity, 1 eq) was added to the mixture and stirred at 30° C. for 11 hrs. The reaction was quenched by adding NaHCO3 aqueous solution (10 mL) and extracted with ethyl acetate (20 mL×3) and washed with saturated brine (10 mL×3). The organic phase was dried over anhydrous sodium sulfate and filtered, and the filtrate was concentrated under reduced pressure to give (Z)-3-((E)-4-aminobut-2-en-1-yl)-4-(3,3-dimethoxypropoxy)-2-((4-ethyl-2-methylthiazole-5-carbonyl)imino)-2,3-dihydrobenzo[d]thiazole-6-carboxamide (2.2 g, 91% purity, 81% yield) as off-white solid. LCMS (ESI+): m/z 534.2 (M+1)+, RT: 1.013 min.

Synthesis of (Z)-3-((E)-4-((4-carbamoyl-2-methoxy-6-nitrophenyl)amino)but-2-en-1-yl)-4-(3,3-dimethoxypropoxy)-2-((4-ethyl-2-methylthiazole-5-carbonyl)imino)-2,3-dihydrobenzo[d]thiazole-6-carboxamide (AB4): Compound AB3 (2.2 g, 3.73 mmol, 91% purity, 1 eq) was dissolved in t-butanol (22 mL). 4-Chloro-3-methoxy-5-nitrobenzamide (1.03 g, 4.48 mmol, 1.2 eq), diisopropylethylamine (2.41 g, 18.65 mmol, 3.25 mL, 5 eq) and sodium bicarbonate (626.83 mg, 7.46 mmol, 2 eq) were serially added to the mixture. The reaction system was heated to 120° C. in a sealed tube and stirred for 6 hrs and then poured into water. The resulting solid was filtered and purified by silica gel column chromatography (ethyl acetate/methanol (v/v)=1/0-10/1) to obtain (Z)-3-((E)-4-((4-carbamoyl-2-methoxy-6-nitrophenyl)amino)but-2-en-1-yl)-4-(3,3-dimethoxypropoxy)-2-((4-ethyl-2-methylthiazole-5-carbonyl)imino)-2,3-dihydrobenzo[d]thiazole-6-carboxamide, 4 (1.7 g, 84% purity, 52% yield) as orange solid. LCMS (ESI+): m/z 728.0 (M+1)+, RT: 1.181 min.

Synthesis of (Z)-3-((E)-4-((2-amino-4-carbamoyl-6-methoxyphenyl)amino)but-2-en-1-yl)-4-(3,3-dimethoxypropoxy)-2-((4-ethyl-2-methylthiazole-5-carbonyl)imino)-2,3-dihydrobenzo[d]thiazole-6-carboxamide (AB5): Compound AB4 (1.7 g, 1.95 mmol, 84% purity, 1 eq) was dissolved in methanol (17 mL). An aqueous solution (1.7 mL) of sodium dithionite (4.07 g, 23.40 mmol, 12 eq) and aqueous ammonia (7.59 g, 58.51 mmol, 8.35 mL, 27% aqueous solution, 30 eq) were serially added to the mixture. The reaction system was stirred at 25° C. for 2 hrs. After the reaction was completed, the mixture was diluted with methanol (10 mL) and ethyl acetate (100 mL) and washed with saturated brine (30 mL×3). The organic phase was dried over anhydrous sodium sulfate and filtered, and the filtrate was concentrated under reduced pressure to give (Z)-3-((E)-4-((2-amino-4-carbamoyl-6-methoxyphenyl)amino)but-2-en-1-yl)-4-(3,3-dimethoxypropoxy)-2-((4-ethyl-2-methylthiazole-5-carbonyl)imino)-2,3-dihydrobenzo[d]thiazole-6-carboxamide, 5 (1.5 g, 69% purity, 76% yield) as yellow solid. LCMS (ESI+): m/z 698.1 (M+1)+, RT: 1.106 min.

Synthesis of (Z)-3-((E)-4-(5-carbamoyl-2-(1-ethyl-3-methyl-1H-pyrazole-5-carboxamido)-7-methoxy-1H-benzo[d]imidazol-1-yl)but-2-en-1-yl)-4-(3,3-dimethoxypropoxy)-2-((4-ethyl-2-methylthiazole-5-carbonyl)imino)-2,3-dihydrobenzo[d]thiazole-6-carboxamide (AB6): Compound AB5 (1.5 g, 69% purity, 1.49 mmol, 1 eq) was dissolved in N, N-dimethylformamide (15 mL). 2-ethyl-5-methyl-pyrazole-3-carbonyl isothiocyanate (581.68 mg, 2.98 mmol, 2 eq), then 1-ethyl-(3-dimethylaminopropyl)carbonyldiimine hydrochloride (856.69 mg, 4.47 mmol, 3 eq) and triethylamine (753.68 mg, 7.45 mmol, 1.04 mL, 5 eq) were added to the mixture. The reaction system was stirred at 25° C. for 2 hrs. The reaction was quenched by adding water (30 mL), and the resulting solid was filtered, washed with water (10 mL) and dried to afford (Z)-3-((E)-4-(5-carbamoyl-2-(1-ethyl-3-methyl-1H-pyrazole-5-carboxamido)-7-methoxy-1H-benzo[d]imidazol-1-yl)but-2-en-1-yl)-4-(3,3-dimethoxypropoxy)-2-((4-ethyl-2-methylthiazole-5-carbonyl)imino)-2,3-dihydrobenzo[d]thiazole-6-carboxamide, 6 (800 mg, 82% purity, 51% yield) as white solid. LCMS (ESI+): m/z 859.1 (M+1)+, RT: 1.181 min.

Synthesis of (Z)-3-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-((4-ethyl-2-methylthiazole-5-carbonyl)imino)-4-hydroxy-2,3-dihydrobenzo[d]thiazole-6-carboxamide (AB7): Compound AB6 (600 mg, 82% purity, 569.97 μmol, 1 eq) was dissolved in tetrahydrofuran (3 mL). 1 M hydrochloric acid aqueous solution (3 mL) was added dropwise to the mixture. The reaction system was stirred at 25° C. for 2 hrs. The reaction was adjusted to pH=7 with sodium bicarbonate aqueous solution, and the resulting solid was filtered, washed with water (10 mL) and dried to afford (Z)-3-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-((4-ethyl-2-methylthiazole-5-carbonyl)imino)-4-hydroxy-2,3-dihydrobenzo[d]thiazole-6-carboxamide, 7 (220 mg, 81% purity, 41% yield) as white solid. LCMS (ESI+): m/z: 757.1 (M+1)+, RT: 1.074 min.

Synthesis of tert-butyl 3-[1-[3-[[(2Z)-6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-(4-ethyl-2-methyl-thiazole-5-carbonyl)imino-1,3-benzothiazol-4-yl]oxy]propyl]-4-piperidyl]-6,8-dihydro-5H-[1,2,4]triazolo[4,3-a]pyrazine-7-carboxylate (AB8): To a solution of Compound AB7 (215 mg, 284.07 μmol, 1 eq) and SV-001226_11D (162.05 mg, 340.88 μmol, 1.2 eq) in DMF (2 mL) was added K2CO3 (117.78 mg, 852.21 μmol, 3 eq). The mixture was stirred at 60° C. for 2 hrs. The reaction mixture was quenched by addition H2O (5 mL), and then extracted with ethyl acetate (2 mL×3). The combined organic layers were washed with brine (3 mL), dried over Na2SO4, filtered and concentrated under reduced pressure to give a residue. The residue was purified by prep-HPLC column: Phenomenex Luna C18 80×30 mm×3 um; mobile phase: [H2O (0.1% TFA)-ACN]; gradient: 20%-50% B over 8.0 min to give tert-butyl 3-[1-[3-[[(2Z)-6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-(4-ethyl-2-methyl-thiazole-5-carbonyl)imino-1,3-benzothiazol-4-yl]oxy]propyl]-4-piperidyl]-6,8-dihydro-5H-[1,2,4]triazolo[4,3-a]pyrazine-7-carboxylate, 8 (150 mg, 92% purity, 47% yield) as white solid. LCMS (ESI+): m/z 1104.7 (M+1)+, R^{T}: 1.621 min.

Synthesis of (2Z)-3-[(E)-4-[5-carbamoyl-2-[(2-ethyl-5-methyl-pyrazole-3-carbonyl)amino]-7-methoxy-benzimidazol-1-yl]but-2-enyl]-2-(4-ethyl-2-methyl-thiazole-5-carbonyl)imino-4-[3-[4-[(Z)—N-[2-(methylamino)ethyl]carbamohydrazonoyl]-1-piperidyl]propoxy]-1,3-benzothiazole-6-carboxamide (Compound 86, AB9): To a solution of Compound AB8 (150 mg, 1.37 mmol, 1 eq) in dichloromethane (1.425 mL) and trifluoroacetic acid (0.075 mL). The mixture was stirred at 25° C. for 12 hrs. LCMS indicated all reactant was consumed and product with desired mass was detected. The reaction mixture was concentrated under reduced pressure to give a residue. The residue was purified by prep-HPLC (column: 3_Phenomenex Luna C18 75×30 mm×3 um; mobile phase: [H2O (0.1% TFA)-ACN]; gradient: 15%-50% B over 8.0 min) to give (2Z)-3-[(E)-4-[5-carbamoyl-2-[(2-ethyl-5-methyl-pyrazole-3-carbonyl)amino]-7-methoxy-benzimidazol-1-yl]but-2-enyl]-2-(4-ethyl-2-methyl-thiazole-5-carbonyl)imino-4-[3-[4-[(Z)—N-[2-(methylamino)ethyl]carbamohydrazonoyl]-1-piperidyl]propoxy]-1,3-benzothiazole-6-carboxamide, 9 (71.5 mg, 100% purity, 52% yield) as white solid. 1H NMR (400 MHz, DMSO-d6) δ ppm 12.56-13.26 (m, 1H) 9.55-9.79 (m, 1H) 9.43-9.54 (m, 1H) 7.94-8.13 (m, 3H) 7.63 (s, 1H) 7.48-7.60 (m, 2H) 7.38 (br s, 1H) 7.26-7.35 (m, 1H) 6.44-6.55 (m, 1H) 5.77-5.88 (m, 2H) 5.30 (brs, 2H) 4.90 (brs, 2H) 4.45-4.60 (m, 5H) 4.19 (br t, J=5.54 Hz, 2H) 4.07-4.14 (m, 2H) 3.71-3.81 (m, 3H) 3.62-3.70 (m, 2H) 3.50 (br d, J=11.32 Hz, 2H) 3.22 (br s, 1H) 3.00-3.12 (m, 3H) 2.82-2.97 (m, 2H) 2.52-2.56 (m, 3H) 1.91-2.10 (m, 9H) 1.23-1.30 (m, 3H) 1.17 (t, J=7.51 Hz, 3H). LCMS (ESI+): m/z 1004.3 (M+1)+, RT: 2.025 min.

### Synthesis 2AC. Compound 25

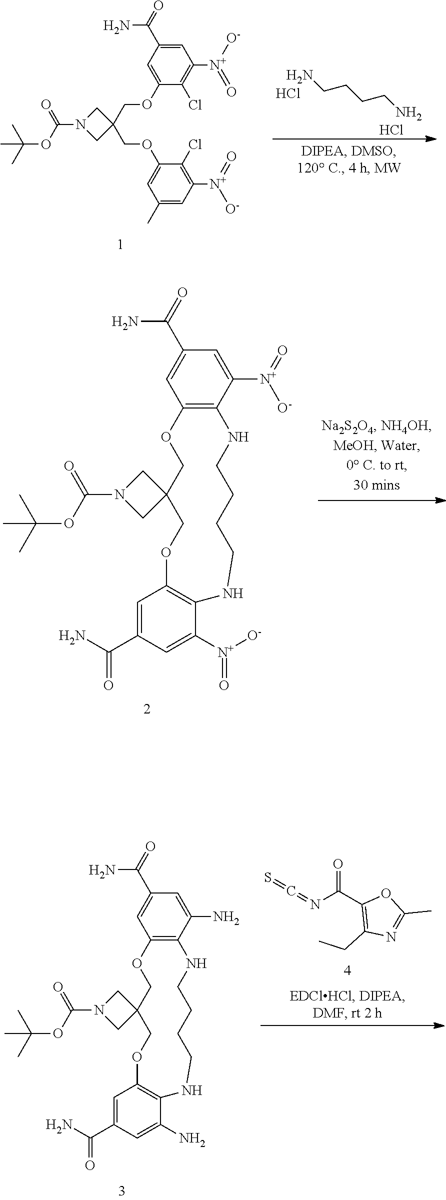

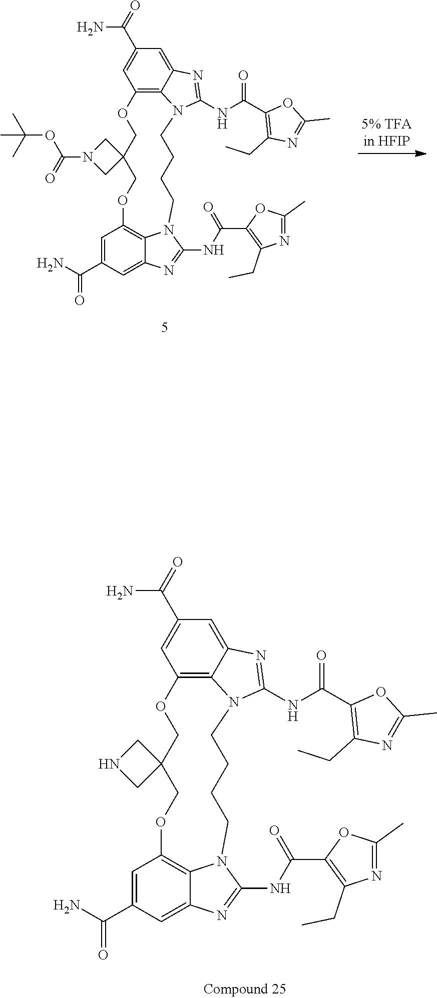 [SMILES: CCc1nc(C)oc1C(=O)Nc1nc2cc(C(N)=O)cc3c2n1CCCCn1c(NC(=O)c2oc(C)nc2CC)nc2cc(C(N)=O)cc(c21)OCC1(CO3)CN(C(=O)OC(C)(C)C)C1 ; CCc1nc(C)oc1C(=O)Nc1nc2cc(C(N)=O)cc3c2n1CCCCn1c(NC(=O)c2oc(C)nc2CC)nc2cc(C(N)=O)cc(c21)OCC1(CNC1)CO3]

Synthesis of tert-butyl 9,22-dicarbamoyl-11,20-dinitro-spiro[2,6-dioxa-13,18-diazatricyclo[17.4.0.07,12]tricosa-1 (19), 7 (12), 8,10,20,22-hexaene-4,3′-azetidine]-1′-carboxylate (AC2): In a sealed tube vessel, a suspension of tert-butyl 3,3-bis[(5-carbamoyl-2-chloro-3-nitro-phenoxy)methyl]azetidine-1-carboxylate (0.5 g, 0.81 mmol), DIPEA (0.68 mL, 4.88 mmol) and putrescine (107.61 mg, 1.22 mmol) in DMSO (2.5 mL) was heated at at 120° C. for 16h. Crude was purified by prep-HPLC (Column: Phenomenex kinetex 5 μm C18 100 Å, 250×50 mm and using 10-70% ACN: 10 mM NH4OAc in water as eluent, Flow rate: 118 mL/min, run time: 30 mins to afford tert-butyl 9,22-dicarbamoyl-11,20-dinitro-spiro[2,6-dioxa-13,18-diazatricyclo[17.4.0.07,12]tricosa-1 (19), 7 (12), 8,10,20,22-hexaene-4,3′-azetidine]-1′-carboxylate (400 mg, 0.6353 mmol, 78.06400 yield) as brown solid. LC-MS [ESI]: Calculated for C₂₈H₃₅N₇O₁₀ [M+H⁺]: 630.2, Found: 630.4.

Synthesis of tert-butyl 11,20-diamino-9,22-dicarbamoyl-spiro[12,6-dioxa-13,18-diazatricyclo[17.4.0.07,12]tricosa-1 (19), 7 (12), 8,10,20,22-hexaene-4,3′-azetidine]-1′-carboxylate (AC3): To a stirred suspension of tert-butyl 9,22-dicarbamoyl-11,20-dinitro-spiro[2,6-dioxa-13,18-diazatricyclo[17.4.0.07,12]tricosa-1 (19), 7 (12), 8,10,20,22-hexaene-4,3′-azetidine]-1′-carboxylate, 2 (0.1 g, 0.16 mmol) in Methanol (5 mL) at 0° C. was added Sodium hydrosulfite (276.53 mg, 1.59 mmol) in Water (1 mL) followed by the simultaneous addition of Ammonium hydroxide (1.03 mL, 7.94 mmol). The resulting suspension was allowed to warm to rt and stirred at rt for 2 h. The reaction was complete. Reaction mixture diluted with DCM (80 mL) before it was filtered, solids were washed with 20% MeOH/DCM. Combined filtrate was washed with water, dried over sodium sulfate and concentrated to afford tert-butyl 11,20-diamino-9,22-dicarbamoyl-spiro[2,6-dioxa-13,18-diazatricyclo[17.4.0.07,12]tricosa-1 (19), 7 (12), 8,10,20,22-hexaene-4,3′-azetidine]-1′-carboxylate, 3 (70 mg, 0.1229 mmol, 77.369% yield) as white solid. Crude was used in next step without further purification.

Synthesis of tert-butyl 11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-7,9,11,13 (27), 19 (26), 20,22,24-octaene-16,3′-azetidine]-1′-carboxylate (AC5): To a stirred solution of tert-butyl 11,20-diamino-9,22-dicarbamoyl-spiro[2,6-dioxa-13,18-diazatricyclo[17.4.0.07,12]tricosa-1 (19), 7 (12), 8,10,20,22-hexaene-4,3′-azetidine]-1′-carboxylate, 3 (30. mg, 0.05 mmol) in DMF (1.5 mL) at rt was added 4-ethyl-2-methyl-oxazole-5-carbonyl isothiocyanate, 4 (31. mg, 0.16 mmol) followed by the addition of DIPEA (0.05 mL, 0.38 mmol). Reaction mixture was stirred at rt for 30 mins. To this was added EDCI·HCl (36.35 mg, 0.19 mmol) and the resulting solution was stirred at rt for 2 days. Reaction mixture was purified by preparative HPLC (Column: Phenomenex kinetex 5 μm C18 100 Å, 250×21.2 mm and using 10-70% ACN: 0.1% TFA in water as eluent, Flow rate: 21.2 mL/min, run time: 30 mins to afford tert-butyl 11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-7,9,11,13 (27), 19 (26), 20,22,24-octaene-16,3′-azetidine]-1′-carboxylate, 5 (22 mg, 0.0246 mmol, 46.73% yield) as white solid. LC-MS [ESI]: Calculated for C₄₄H₅₁N₁₁O₁₀[M+H⁺]: 894.4, Found: 894.4.

Synthesis of 7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-7,9,11,13 (27), 19 (26), 20,22,24-octaene-16,3′-azetidine]-11,21-dicarboxamide (Compound 25, AC6): To a stirred suspension of tert-butyl 11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-7,9,11,13 (27), 19 (26), 20,22,24-octaene-16,3′-azetidine]-1′-carboxylate, 5 (22. mg, 0.02 mmol) in HFIP (1 mL) at rt was added TFA (0.75 mL, 0.49 mmol) (5% in HFIP). The resulting solution was stirred at rt for 2h. Reaction mixture was concentrated under reduced pressure and purified by preparative HPLC (Column: Phenomenex kinetex 5 μm C18 100 Å, 250×21.2 mm and using 5-50% ACN: 1% TFA in water as eluent, Flow rate: 21.2 mL/min, run time: 30 mins to afford 7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-7,9,11,13 (27), 19 (26), 20,22,24-octaene-16,3′-azetidine]-11,21-dicarboxamide, 6 (12 mg, 0.0151 mmol, 61.425% yield) as white solid. 1H NMR (400 MHz, DMSO) δ 9.07 (s, 2H), 7.99 (s, 2H), 7.70 (d, J=1.3 Hz, 2H), 7.57 (d, J=1.4 Hz, 2H), 7.41 (s, 2H), 4.70 (s, 4H), 4.29 (d, J=7.5 Hz, 4H), 4.20 (t, J=6.2 Hz, 4H), 2.92 (q, J=7.6 Hz, 4H), 2.42 (s, 6H), 1.98 (s, 4H), 1.11 (t, J=7.5 Hz, 6H). LC-MS [ESI]: Calculated for C₃₉H₄₃N₁₁O₈[M+H+]: 794.3, Found: 794.4.

### Synthesis 2AD. Compound 87

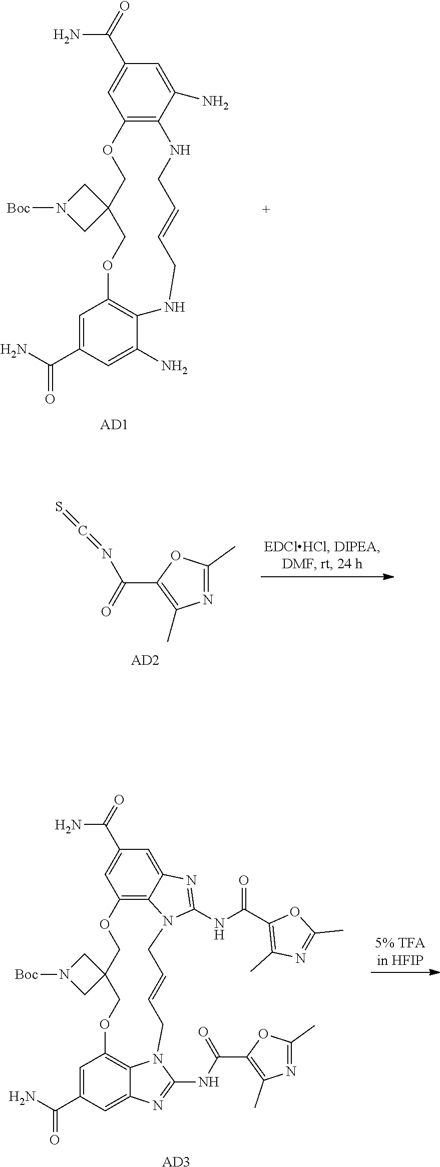

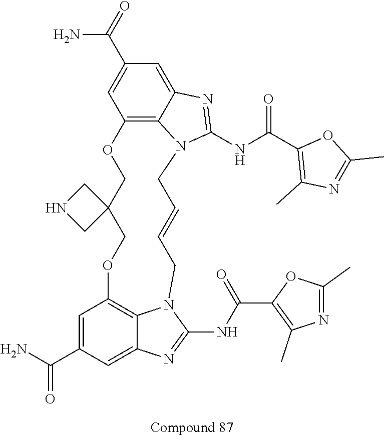 [SMILES: Cc1nc(C)c(C(=O)Nc2nc3cc(C(N)=O)cc4c3n2C/C=C/Cn2c(NC(=O)c3oc(C)nc3C)nc3cc(C(N)=O)cc(c32)OCC2(CNC2)CO4)o1]

Synthesis of tert-butyl (3E)-11,21-dicarbamoyl-7,25-bis[(2,4-dimethyloxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (AD3): To a stirred solution of tert-butyl (15E)-11,20-diamino-9,22-dicarbamoyl-spiro[2,6-dioxa-13,18-diazatricyclo[17.4.0.07,12]tricosa-1 (19), 7 (12), 8,10,15,20,22-heptaene-4,3′-azetidine]-1′-carboxylate (39.99 mg, 0.07 mmol) in DMF (1.5 uL) at rt was added 2,4-dimethyloxazole-5-carbonyl isothiocyanate (38.5 mg, 0.21 mmol) followed by the addition of DIPEA (0.07 mL, 0.51 mmol). Reaction mixture was stirred at rt for 2h. To this was added EDCI·HCl (48.61 mg, 0.25 mmol) and reaction was continued for 18h. Reaction mixture was purified by preparative HPLC (Column: Phenomenex kinetex 5 μm C18 100 Å, 250×21.2 mm and using 10-70% ACN: 0.1% TFA in water as eluent, Flow rate: 21.2 mL/min, run time: 30 mins to afford tert-butyl (3E)-11,21-dicarbamoyl-7,25-bis[(2,4-dimethyloxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (13 mg, 0.0150 mmol, 21.363% yield) as white solid. LC-MS [ESI]: Calculated for C₄₂H₄₅N₁₁O₁₀[M+H⁺]: 864.3, Found: 864.5.

Synthesis of (3E)-7,25-bis[(2,4-dimethyloxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-11,21-dicarboxamide (AD4): To a stirred solution of tert-butyl (3E)-11,21-dicarbamoyl-7,25-bis[(2,4-dimethyloxazole-5-carbonyl)amino]spiro[14,18-dioxa-1,6,8,24-tetrazapentacyclo[17.6.1.16,9.023,26.013,27]-heptacosa-3,7,9,11,13 (27), 19 (26), 20,22,24-nonaene-16,3′-azetidine]-1′-carboxylate, (11. mg, 0.01 mmol) in HFIP (0.5 mL) at rt was added TFA (0.39 mL, 0.25 mmol) (5% in HFIP). The resulting solution was stirred at rt for 2h. Reaction mixture was purified by preparative HPLC (Column: Phenomenex kinetex 5 μm C18 100 Å, 250×21.2 mm and using 5-50% ACN: 0.1% TFA in water as eluent, Flow rate: 21.2 mL/min, run time: 30 mins to afford to afford (3E)-7,25-bis[(2,4-dimethyloxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-11,21-dicarboxamide (4 mg, 0.0052 mmol, 41.13% yield) as white solid. 1H NMR (400 MHz, DMSO) δ 12.85 (s, 2H), 9.02 (s, 2H), 7.98 (s, 2H), 7.76 (s, 2H), 7.72 (s, 2H), 7.45 (s, 2H), 5.42 (s, 2H), 4.85 (s, 5H), 4.54 (s, 5H), 4.08 (t, J=5.9 Hz, 5H), 2.37 (s, 6H), 2.32 (s, 7H). LC-MS [ESI]: Calculated for C₃₇H₃₇N₁₁O₈[M+H+]: 764.3, Found: 764.4.

### Synthesis 2AE. Compound 64

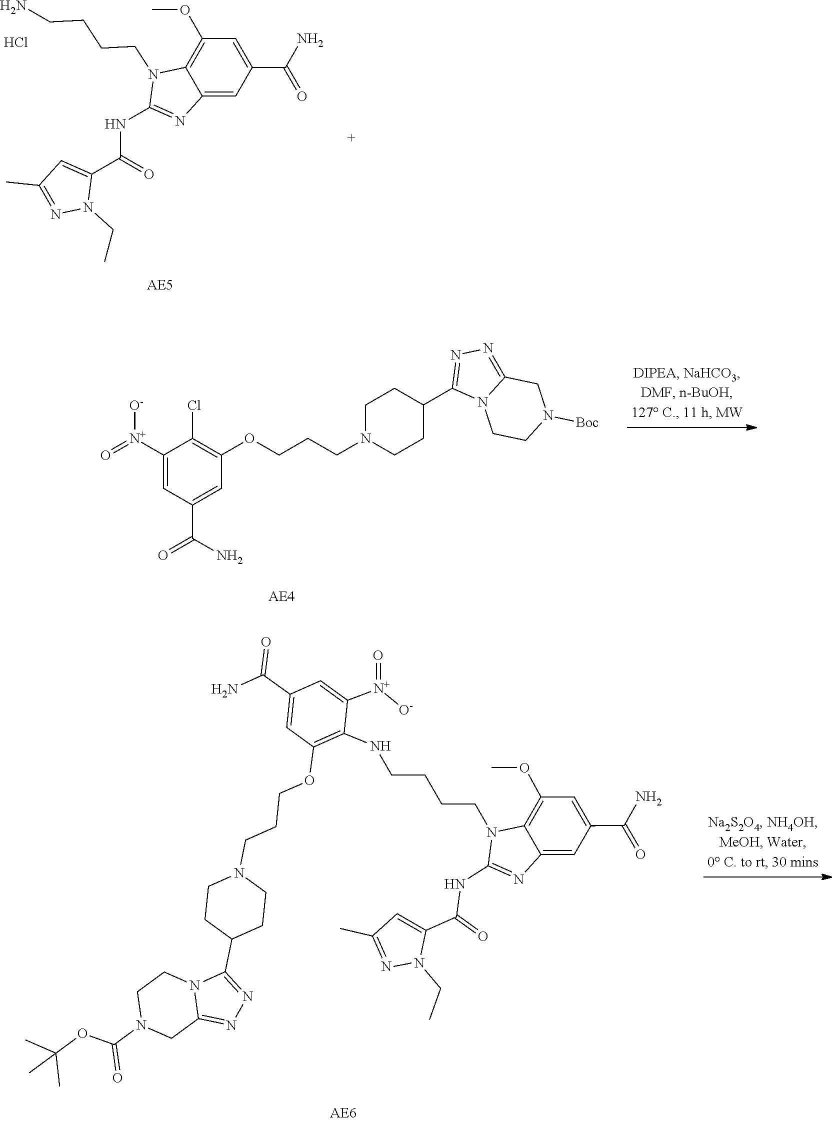 [SMILES: CCn1nc(C)cc1C(=O)Nc1nc2cc(C(N)=O)cc(OC)c2n1CCCCN ; Cl ; CC(C)(C)OC(=O)N1CCn2c(nnc2C2CCN(CCCOc3cc(C(N)=O)cc([N+](=O)[O-])c3Cl)CC2)C1 ; CCn1nc(C)cc1C(=O)Nc1nc2cc(C(N)=O)cc(OC)c2n1CCCCNc1c(OCCCN2CCC(c3nnc4n3CCN(C(=O)OC(C)(C)C)C4)CC2)cc(C(N)=O)cc1[N+](=O)[O-]]

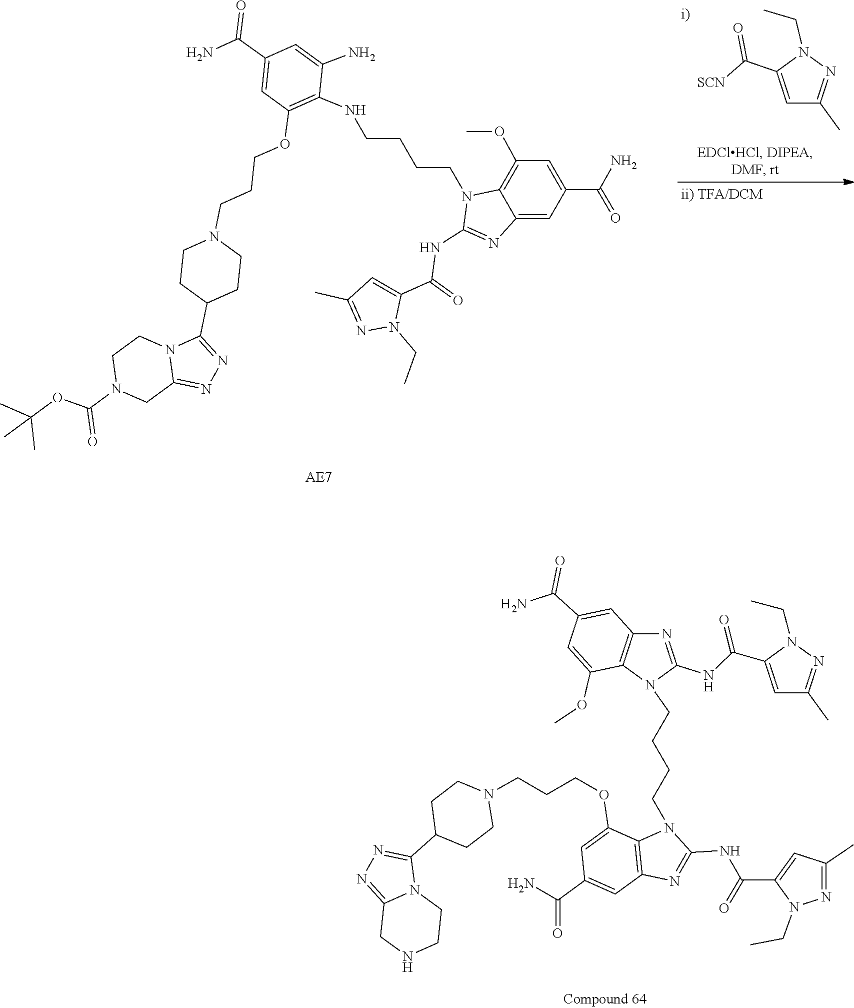

Synthesis of tert-butyl 3-[1-[3-[5-carbamoyl-2-[4-[5-carbamoyl-2-[(2-ethyl-5-methyl-pyrazole-3-carbonyl)amino]-7-methoxy-benzimidazol-1-yl]butylamino]-3-nitro-phenoxy]propyl]-4-piperidyl]-6,8-dihydro-5H-[1,2,4]triazolo[4,3-a]pyrazine-7-carboxylate (AE6): A stirred suspension of tert-butyl 3-[1-[3-(5-carbamoyl-2-chloro-3-nitro-phenoxy)propyl]-4-piperidyl]-6,8-dihydro-5H-[1,2,4]triazolo[4,3-a]pyrazine-7-carboxylate (1.5 g, 2.66 mmol), 1-(4-aminobutyl)-2-[(2-ethyl-5-methyl-pyrazole-3-carbonyl)amino]-7-methoxy-benzimidazole-5-carboxamide; dihydrochloride (1681.61 mg, 3.46 mmol), Sodium bicarbonate (446.82 mg, 5.32 mmol) and DIPEA (1.85 mL, 10.64 mmol) in 1-Butanol (10 mL) was heated to 120° C. for 48h. LC-MS showed major desired product formation. Reaction mixture was concentrated under reduced pressure before it was suspended in DMF and filtered. Filtrate was purified by prep-HPLC (Column: Phenomenex kinetex 5 m C18 100 Å, 250×50 mm and using 5-40% ACN: 1% TFA in water as eluent, Flow rate: 118.1 mL/min, run time: 25 mins to afford tert-butyl 3-[1-[3-[5-carbamoyl-2-[4-[5-carbamoyl-2-[(2-ethyl-5-methyl-pyrazole-3-carbonyl)amino]-7-methoxy-benzimidazol-1-yl]butylamino]-3-nitro-phenoxy]propyl]-4-piperidyl]-6,8-dihydro-5H-[1,2,4]triazolo[4,3-a]pyrazine-7-carboxylate (700 mg, 0.7438 mmol, 27.97% yield) as white solid. LC-MS [ESI]: Calculated for C₄₅H₆₀N₁₄O₉[M+H⁺]: 941.47, Found: 941.68.

Synthesis of tert-butyl 3-[1-[3-[3-amino-5-carbamoyl-2-[4-[5-carbamoyl-2-[(2-ethyl-5-methyl-pyrazole-3-carbonyl)amino]-7-methoxy-benzimidazol-1-yl]butylamino]phenoxy]propyl]-4-piperidyl]-6,8-dihydro-5H-[1,2,4]triazolo[4,3-a]pyrazine-7-carboxylate (AE7): To a stirred suspension of tert-butyl 3-[1-[3-[5-carbamoyl-2-[4-[5-carbamoyl-2-[(2-ethyl-5-methyl-pyrazole-3-carbonyl)amino]-7-methoxy-benzimidazol-1-yl]butylamino]-3-nitro-phenoxy]propyl]-4-piperidyl]-6,8-dihydro-5H-[1,2,4]triazolo[4,3-a]pyrazine-7-carboxylate (500. mg, 0.53 mmol) in Methanol (10 mL) at rt were added Na2S2O4 (462.54 mg, 2.66 mmol) in Water (2 mL) and Ammonium Hydroxide (0.69 mL, 5.32 mmol). The resulting suspension was stirred at rt for 2h. Solids were filtered and washed with MeOH. Filtrate was concentrated and the crude was suspended into water and 10% MeOH/DCM (1:1). Two layers were separated, aqueous was back extracted with 10% MeOH/DCM (2×20 mL). Combined organic part was dried over sodium sulfate and concentrated to afford tert-butyl 3-[1-[3-[3-amino-5-carbamoyl-2-[4-[5-carbamoyl-2-[(2-ethyl-5-methyl-pyrazole-3-carbonyl)amino]-7-methoxy-benzimidazol-1-yl]butylamino]phenoxy]propyl]-4-piperidyl]-6,8-dihydro-5H-[1,2,4]triazolo[4,3-a]pyrazine-7-carboxylate (300 mg, 0.3293 mmol, 61.974% yield) as colorless gum. Crude was used in the next step without further purification. LC-MS [ESI]: Calculated for C₄₅H₆₂N₁₄O₇[M+H⁺]: 911.5, Found: 911.7.

Synthesis of 1-[4-[5-carbamoyl-2-[(2-ethyl-5-methyl-pyrazole-3-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]butyl]-2-[(2-ethyl-5-methyl-pyrazole-3-carbonyl)amino]-7-methoxy-benzimidazole-5-carboxamide (Compound 64):

A solution of tert-butyl 3-[1-[3-[3-amino-5-carbamoyl-2-[4-[5-carbamoyl-2-[(2-ethyl-5-methyl-pyrazole-3-carbonyl)amino]-7-methoxy-benzimidazol-1-yl]butylamino]phenoxy]propyl]-4-piperidyl]-6,8-dihydro-5H-[1,2,4]triazolo[4,3-a]pyrazine-7-carboxylate (300. mg, 0.33 mmol), 2-ethyl-5-methyl-pyrazole-3-carbonyl isothiocyanate (70.72 mg, 0.36 mmol), and DIPEA (0.17 mL, 0.99 mmol) in DMF (4.5 mL) was stirred at rt for 2h. To this was added 3-(ethyliminomethyleneamino)-N,N-dimethyl-propan-1-amine; hydrochloride (94.68 mg, 0.49 mmol) and the resulting solution was stirred at 40 C for 18h. Reaction mixture was purified by prep-HPLC (Column: Phenomenex kinetex 5 m C18 100 Å, 250×50 mm and using 5-40% ACN: 0.1% TFA in water as eluent, Flow rate: 118.1 mL/min, run time: 25 mins to afford tert-butyl 3-[1-[3-[6-carbamoyl-3-[4-[5-carbamoyl-2-[(2-ethyl-5-methyl-pyrazole-3-carbonyl)amino]-7-methoxy-benzimidazol-1-yl]butyl]-2-[(2-ethyl-5-methyl-pyrazole-3-carbonyl)amino]benzimidazol-4-yl]oxypropyl]-4-piperidyl]-6,8-dihydro-5H-[1,2,4]triazolo[4,3-a]pyrazine-7-carboxylate (110 mg, 0.1026 mmol, 31.155% yield) as white solid. LC-MS [ESI]: Calculated for C₅₃H₆₉N₁₇O₈[M+H+]: 1072.56, Found: 1072.62.

A stirred solution of crude tert-butyl 3-[1-[3-[6-carbamoyl-3-[4-[5-carbamoyl-2-[(2-ethyl-5-methyl-pyrazole-3-carbonyl)amino]-7-methoxy-benzimidazol-1-yl]butyl]-2-[(2-ethyl-5-methyl-pyrazole-3-carbonyl)amino]benzimidazol-4-yl]oxypropyl]-4-piperidyl]-6,8-dihydro-5H-[1,2,4]triazolo[4,3-a]pyrazine-7-carboxylate (110. mg, 0.1 mmol) in HFIP (1.5 mL) at rt was added TFA (3.14 mL, 2.05 mmol) (5% in HFIP). Reaction mixture was stirred at this temperature for 2h before it was concentrated under reduced pressure and was purified by prep-HPLC (Column: Phenomenex kinetex 5 μm C18 100 Å, 250×50 mm and using 5-40% ACN: 1% TFA in water as eluent, Flow rate: 118.1 mL/min, run time: 25 mins to afford 1-[4-[5-carbamoyl-2-[(2-ethyl-5-methyl-pyrazole-3-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]butyl]-2-[(2-ethyl-5-methyl-pyrazole-3-carbonyl)amino]-7-methoxy-benzimidazole-5-carboxamide (70 mg, 0.0720 mmol, 70.19% yield) as white solid. 1H NMR (400 MHz, DMSO) δ 12.78 (s, 2H), 9.53 (d, J=49.3 Hz, 2H), 8.00 (s, 2H), 7.59-7.51 (m, 2H), 7.38 (s, 2H), 7.33 (t, J=1.6 Hz, 2H), 6.62 (d, J=2.2 Hz, 2H), 4.67-4.51 (m, 7H), 4.37 (d, J=12.8 Hz, 6H), 4.18 (t, J=5.7 Hz, 9H), 3.84 (d, J=5.3 Hz, 4H), 3.67 (d, J=6.1 Hz, 3H), 3.50 (d, J=11.8 Hz, 2H), 3.05 (s, 1H), 2.92 (d, J=11.2 Hz, 2H), 2.15-2.02 (m, 9H), 1.92 (d, J=36.6 Hz, 6H), 1.33 (td, J=7.1, 4.3 Hz, 6H). LC-MS [ESI]: Calculated for C₄₈H₆₁N₁₇O₆[M+H+]: 972.5, Found: 972.4.

### Synthesis 2AF. Compound 47

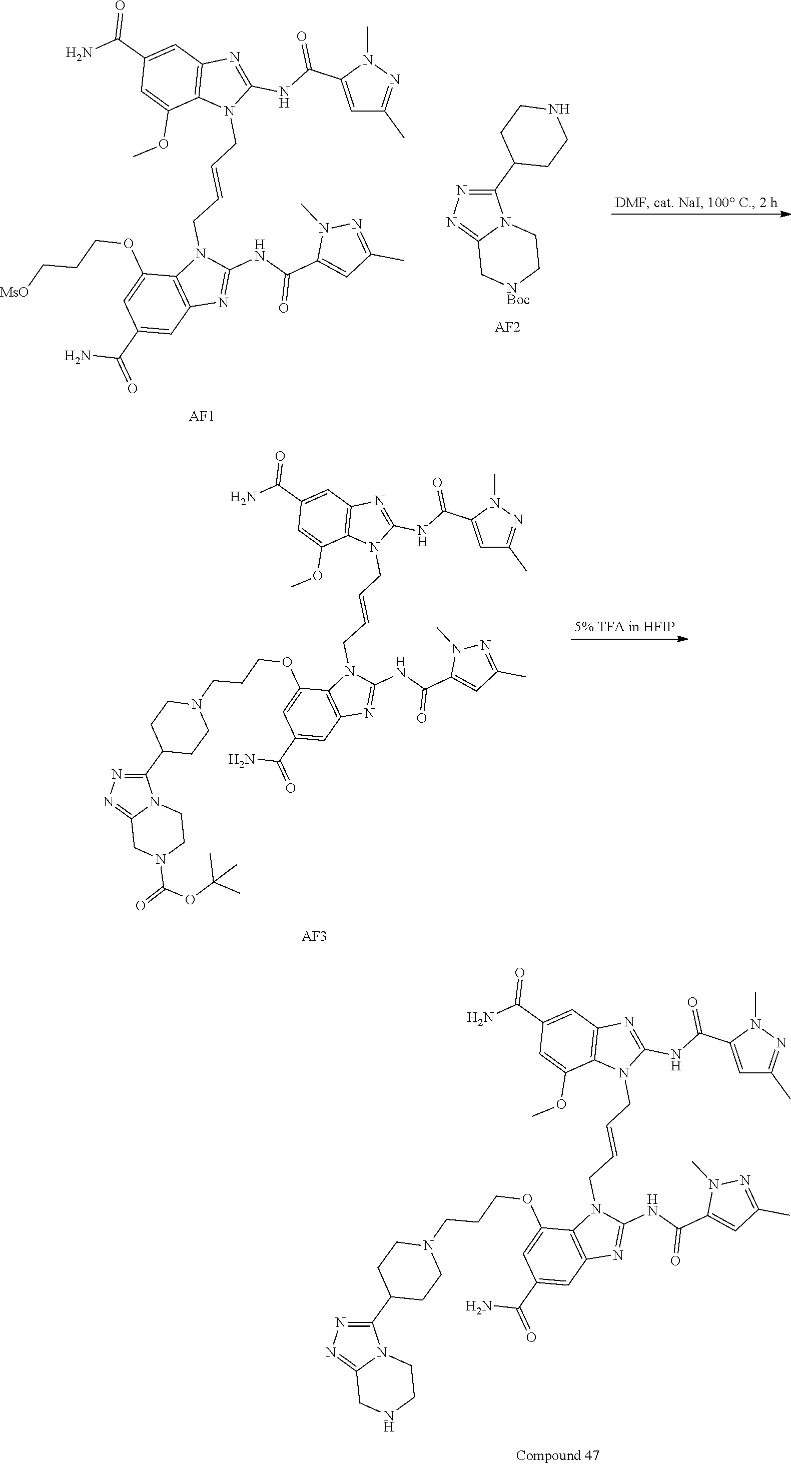

Synthesis of tert-butyl 3-[1-[3-[6-carbamoyl-3-[(E)-4-[5-carbamoyl-2-[(2,5-dimethylpyrazole-3-carbonyl)amino]-7-methoxy-benzimidazol-1-yl]but-2-enyl]-2-[(2,5-dimethylpyrazole-3-carbonyl)amino]benzimidazol-4-yl]oxypropyl]-4-piperidyl]-6,8-dihydro-5H-[1,2,4]triazolo[4,3-a]pyrazine-7-carboxylate (AF3): A stirred solution of (E)-3-((5-carbamoyl-1-(4-(5-carbamoyl-2-(1,3-dimethyl-1H-pyrazole-5-carboxamido)-7-methoxy-1H-benzo[d]imidazol-1-yl)but-2-en-1-yl)-2-(1,3-dimethyl-1H-pyrazole-5-carboxamido)-1H-benzo[d]imidazol-7-yl)oxy)propyl methanesulfonate (0.16 g, 0.193 mmol, 1 equiv.) in DMF (6.00 mL) was stirred for 5 min. To this at rt were added tert-butyl 3-(piperidin-4-yl)-5,6-dihydro-[1,2,4]triazolo[4,3-a]pyrazine-7 (8H)-carboxylate (0.065 g, 0.212 mmol, 1.1 equiv.) and Sodium iodide (0.003 g, 0.019 mmol, 0.1 equiv.). Reaction mixture was heated to 100° C. and maintained for 2 h. Reaction mixture was diluted with water (10.0 mL) and extracted with 20% MeOH:DCM (3×30.0 mL). Separated both the layers, organic layer was dried on sodium sulphate and concentrated under reduced pressure to afforded desired brown oil compound. Crude was purified by prep-HPLC (YMC ODS AQ C18 (20×250 mm), 5 m Flow: 15.0 mL/min and using 25-90% ACN: 0.1% TFA in water as eluent, Flow rate: 15 mL/min, run time: 25 mins to afford the Boc protected compound 3 (0.012 g, 0.023 mmol, 10.16%). LC-MS [ESI]: Calculated for C₅₁H₆₃N₁₇O₈[M+H+]: 1042.51, Found: 1042.57.

Synthesis of 1-[(E)-4-[5-carbamoyl-2-[(2,5-dimethylpyrazole-3-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]but-2-enyl]-2-[(2,5-dimethylpyrazole-3-carbonyl)amino]-7-methoxy-benzimidazole-5-carboxamide (Compound 47, AF4): To a stirred suspension of tert-butyl 3-[1-[3-[6-carbamoyl-3-[(E)-4-[5-carbamoyl-2-[(2,5-dimethylpyrazole-3-carbonyl)amino]-7-methoxy-benzimidazol-1-yl]but-2-enyl]-2-[(2,5-dimethylpyrazole-3-carbonyl)amino]benzimidazol-4-yl]oxypropyl]-4-piperidyl]-6,8-dihydro-5H-[1,2,4]triazolo[4,3-a]pyrazine-7-carboxylate (10.2 mg, 0.01 mmol) in HFIP (1 mL) at rt was added TFA (0.3 mL, 0.2 mmol) in HFIP. Reaction mixture was stirred at rt for 6h before it was concentrated and purified by prep HPLC (Column: Phenomenex kinetex 5 μm C18 100 Å, 250×50 mm and using 5-40% ACN: 1% TFA in water as eluent, Flow rate: 118.1 mL/min, run time: 25 mins to afford 1-[(E)-4-[5-carbamoyl-2-[(2,5-dimethylpyrazole-3-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]but-2-enyl]-2-[(2,5-dimethylpyrazole-3-carbonyl)amino]-7-methoxy-benzimidazole-5-carboxamide (3.2 mg, 34% yield) as white solid. 1H NMR (400 MHz, DMSO) δ 12.87 (s, 2H), 9.34 (s, 5H), 8.00 (d, J=21.0 Hz, 2H), 7.67 (d, J=8.3 Hz, 2H), 7.40 (s, 2H), 7.32 (d, J=12.2 Hz, 2H), 6.55 (s, 3H), 5.80 (s, 1H), 5.76 (s, 1H), 4.92 (d, J=12.8 Hz, 4H), 4.55 (s, 3H), 4.17 (s, 3H), 4.05 (t, J=12.7 Hz, 8H), 3.73 (s, 3H), 3.65 (s, 3H), 3.12 (s, 3H), 3.03 (s, 2H), 2.88 (d, J=11.7 Hz, 3H), 2.61 (s, 1H), 2.11 (dd, J=7.5, 3.9 Hz, 8H), 1.93 (d, J=12.2 Hz, 5H). LC-MS [ESI]: Calculated for C₄₆H₅₅N₁₇O₆[M+H+]: 942.46, Found: 942.60; tR=1.64 mins.

### Synthesis 2AG. Compound 46

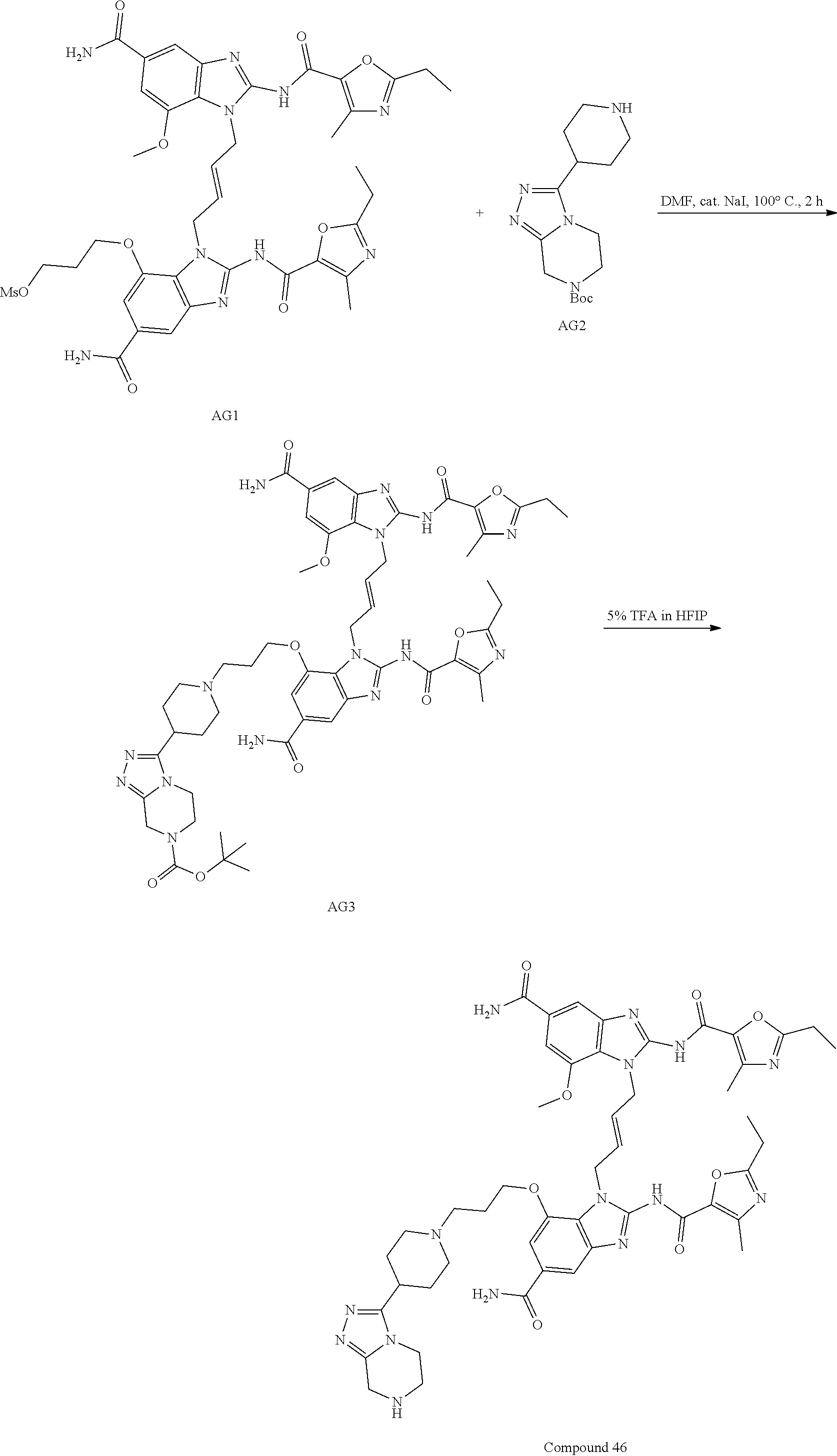

Synthesis of tert-butyl 3-[1-[3-[6-carbamoyl-3-[(E)-4-[5-carbamoyl-2-[(2-ethyl-4-methyl-oxazole-5-carbonyl)amino]-7-methoxy-benzimidazol-1-yl]but-2-enyl]-2-[(2-ethyl-4-methyl-oxazole-5-carbonyl)amino]benzimidazol-4-yl]oxypropyl]-4-piperidyl]-6,8-dihydro-5H-[1,2,4]triazolo[4,3-a]pyrazine-7-carboxylate (AG3): To a stirred solution of E)-3-((5-carbamoyl-1-(4-(5-carbamoyl-2-(2-ethyl-4-methyloxazole-5-carboxamido)-7-methoxy-1H-benzo[d]imidazol-1-yl)but-2-en-1-yl)-2-(2-ethyl-4-methyloxazole-5-carboxamido)-1H-benzo[d]imidazol-7-yl)oxy)propyl methanesulfonate (AG1) (200 mg, 0.232 mmol, 1 equiv.) in N,N-Dimethylformamide (2.000 mL) was added (tert-butyl 3-(piperidin-4-yl)-5,6-dihydro-[1,2,4]triazolo[4,3-a]pyrazine-7 (8H)-carboxylate (85.696 mg, 0.279 mmol, 1.2 equiv.) and stirred at 50° C. for 16 h in a sealed tube. After completion of the reaction, added ice water (10 mL) and extracted the aqueous layer with 20% MeOH in DCM (3×15 mL), combined organic part was dried over sodium sulphate and concentrated in vacuo. Crude was purified by prep HPLC (YMC ODS AQ C18 (20×250 mm), 5 m Flow: 15.0 mL/min and using 25-90% ACN: 0.1% TFA in water as eluent, Flow rate: 15 mL/min, run time: 25 mins to afford tert-butyl (E)-3-(1-(3-((5-carbamoyl-1-(4-(5-carbamoyl-2-(2-ethyl-4-methyloxazole-5-carboxamido)-7-methoxy-1H-benzo[d]imidazol-1-yl)but-2-en-1-yl)-2-(2-ethyl-4-methyloxazole-5-carboxamido)-1H-benzo[d]imidazol-7-yl)oxy)propyl)piperidin-4-yl)-5,6-dihydro-[1,2,4]triazolo[4,3-a]pyrazine-7 (8H)-carboxylate (30 mg, 0.028 mmol, 12.04% yield) as Off white solid. LC-MS [ESI]: Calculated for C53H65N15O10 [M+H+]: 1072.51, Found: 1072.68. indicates 78.12% of desired mass with (M+H)=1072.68 along with 17.43% of de-boc mass with (M+H)=972.65.

Synthesis of N-[5-carbamoyl-1-[(E)-4-[5-carbamoyl-2-[(2-ethyl-4-methyl-oxazole-5-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]but-2-enyl]-7-methoxy-benzimidazol-2-yl]-2-ethyl-4-methyl-oxazole-5-carboxamide (Compound 46): To a stirred suspension of tert-butyl 3-[1-[3-[6-carbamoyl-3-[(E)-4-[5-carbamoyl-2-[(2-ethyl-4-methyl-oxazole-5-carbonyl)amino]-7-methoxy-benzimidazol-1-yl]but-2-enyl]-2-[(2-ethyl-4-methyl-oxazole-5-carbonyl)amino]benzimidazol-4-yl]oxypropyl]-4-piperidyl]-6,8-dihydro-5H-[1,2,4]triazolo[4,3-a]pyrazine-7-carboxylate (27. mg, 0.03 mmol) in HFIP (1 mL) at rt was added TFA (0.77 mL, 0.5 mmol) in HFIP. Reaction mixture was stirred at rt for 6h before it was concentrated and purified by prep HPLC (Column: Phenomenex kinetex 5 μm C18 100 Å, 250×50 mm and using 5-40% ACN: 1% TFA in water as eluent, Flow rate: 118.1 mL/min, run time: 25 mins to afford N-[5-carbamoyl-1-[(E)-4-[5-carbamoyl-2-[(2-ethyl-4-methyl-oxazole-5-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]but-2-enyl]-7-methoxy-benzimidazol-2-yl]-2-ethyl-4-methyl-oxazole-5-carboxamide (14 mg, 0.0144 mmol, 57.192% yield) as white solid. 1H NMR (400 MHz, DMSO) δ 12.77 (s, 2H), 9.57 (d, J=42.5 Hz, 3H), 7.99 (d, J=26.8 Hz, 2H), 7.67 (d, J=4.5 Hz, 2H), 7.40 (s, 2H), 7.32 (d, J=18.9 Hz, 2H), 5.82-5.72 (m, 1H), 5.72-5.63 (m, 1H), 5.00-4.83 (m, 4H), 4.56 (s, 2H), 4.21 (t, J=6.0 Hz, 2H), 4.04 (t, J=5.7 Hz, 2H), 3.76 (s, 4H), 3.67 (t, J=5.9 Hz, 3H), 3.12 (s, 2H), 3.03 (d, J=11.8 Hz, 1H), 2.88 (d, J=11.9 Hz, 2H), 2.74 (dq, J=9.5, 7.6 Hz, 5H), 2.55 (s, 2H), 2.33 (dd, J=30.5, 12.5 Hz, 6H), 2.07 (d, J=13.4 Hz, 2H), 2.01-1.85 (m, 4H), 1.24 (q, J=7.3 Hz, 6H). LC-MS [ESI]: Calculated for C₄₈H₅₇N₁₅O₈[M+H+]: 972.46, Found: 972.60.

### Synthesis 2AH. Compound 88

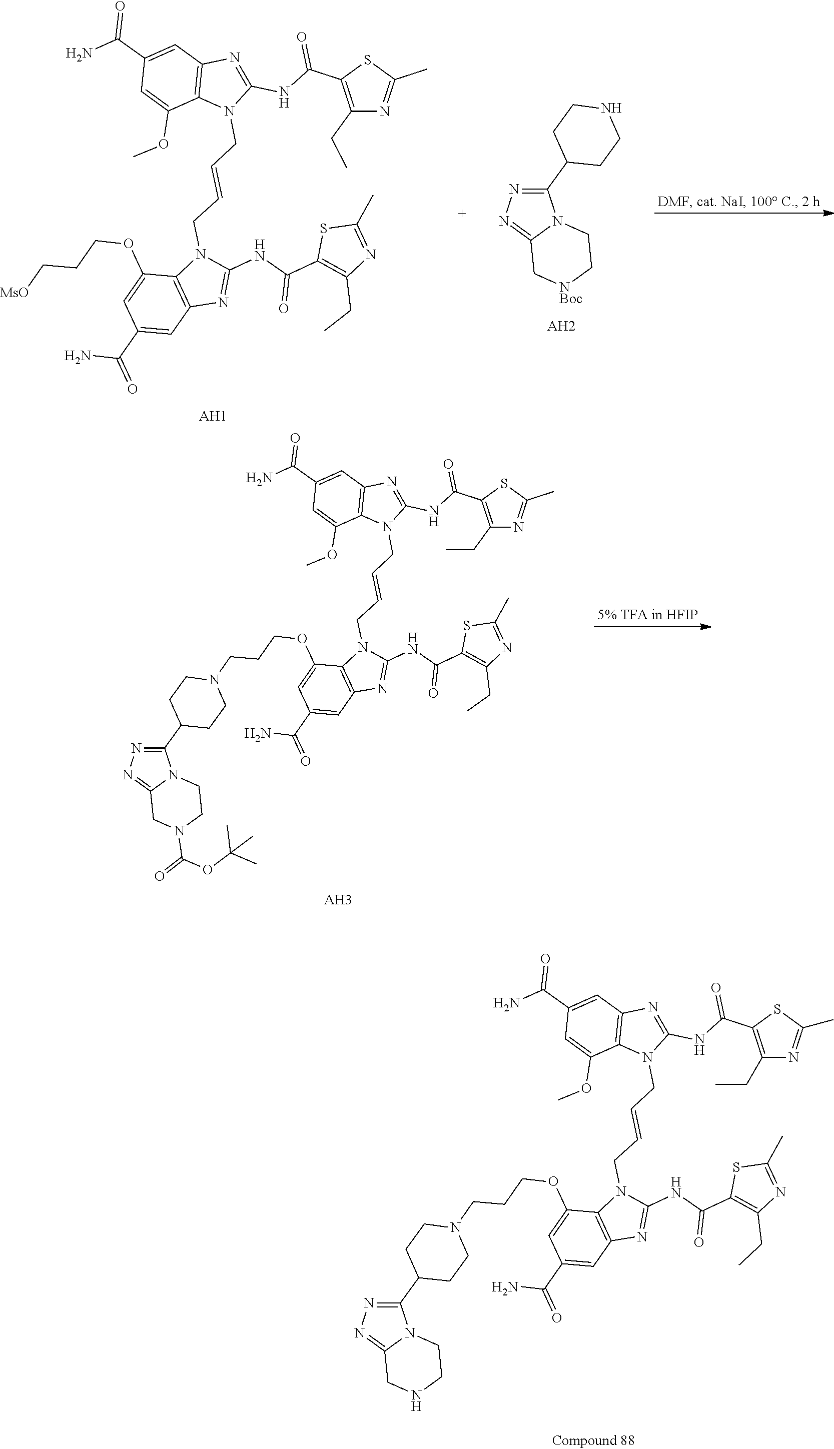

Synthesis of tert-butyl 3-[1-[3-[6-carbamoyl-3-[(E)-4-[5-carbamoyl-2-[(4-ethyl-2-methyl-thiazole-5-carbonyl)amino]-7-methoxy-benzimidazol-1-yl]but-2-enyl]-2-[(4-ethyl-2-methyl-thiazole-5-carbonyl)amino]benzimidazol-4-yl]oxypropyl]-4-piperidyl]-6,8-dihydro-5H-[1,2,4]triazolo[4,3-a]pyrazine-7-carboxylate (AH3): To a stirred solution of (E)-3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl methanesulfonate (300 mg, 0.349 mmol, 1 equiv.) in N,N-dimethylformamide (6.000 mL, 0.384 mmol,) were added tert-butyl 3-(piperidin-4-yl)-5,6-dihydro-[1,2,4]triazolo[4,3-a]pyrazine-7 (8H)-carboxylate (118.102 mg, 0.384 mmol, 1.1 equiv.) and Sodium iodide (5.235 mg, 0.035 mmol, 0.1 equiv.) at rt for 5 min. The reaction mixture was heated to 80° C. for 12 h. The reaction was monitored by TLC and LCMS. The reaction mixture was added cold water (10 mL), and extracted with MeOH in DCM (10%, 100 mL×4), the organic phases were combined, dried over anhydrous sodium sulphate, and filtered, the filtrate was concentrated under reduced pressure, and the crude compound was purified by Pre-HPLC (YMC ODS AQ C18 (20×250 mm), 5 m Flow: 15.0 mL/min and using 25-90% ACN: 0.1% TFA in water as eluent, Flow rate: 15 mL/min, run time: 25 mins to afford tert-butyl 3-[1-[3-[6-carbamoyl-3-[(E)-4-[5-carbamoyl-2-[(4-ethyl-2-methyl-thiazole-5-carbonyl)amino]-7-methoxy-benzimidazol-1-yl]but-2-enyl]-2-[(4-ethyl-2-methyl-thiazole-5-carbonyl)amino]benzimidazol-4-yl]oxypropyl]-4-piperidyl]-6,8-dihydro-5H-[1,2,4]triazolo[4,3-a]pyrazine-7-carboxylate (0.019 g, 0.017 mmol, 9%) as Off white solid. LC-MS [ESI]: Calculated for C53H65N15O8S2 [M+H+]: 1104.47, Found: 1104.63.

Synthesis of N-[5-carbamoyl-1-[(E)-4-[5-carbamoyl-2-[(4-ethyl-2-methyl-thiazole-5-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]but-2-enyl]-7-methoxy-benzimidazol-2-yl]-4-ethyl-2-methyl-thiazole-5-carboxamide; 2,2,2-trifluoroacetic acid (Compound 88, AH4):

To a stirred suspension of tert-butyl 3-[1-[3-[6-carbamoyl-3-[(E)-4-[5-carbamoyl-2-[(4-ethyl-2-methyl-thiazole-5-carbonyl)amino]-7-methoxy-benzimidazol-1-yl]but-2-enyl]-2-[(4-ethyl-2-methyl-thiazole-5-carbonyl)amino]benzimidazol-4-yl]oxypropyl]-4-piperidyl]-6,8-dihydro-5H-[1,2,4]triazolo[4,3-a]pyrazine-7-carboxylate (17.8 mg, 0.016 mmol) in HFIP (1 mL) at rt was added TFA (0.77 mL, 0.5 mmol) in HFIP. Reaction mixture was stirred at rt for 1.5h before it was concentrated and purified by preparative reverse phase HPLC: C18 column, 10-70% acetonitrile in water with 0.1% TFA over 30 min, desired product retention time ˜12 min. 1H NMR (400 MHz, DMSO) δ 12.82 (d, J=7.9 Hz, 2H), 9.53 (s, 2H), 8.01 (s, 1H), 7.95 (s, 1H), 7.66-7.55 (m, 2H), 7.38 (s, 2H), 7.36-7.27 (m, 2H), 5.91-5.73 (m, 2H), 4.87 (dd, J=16.5, 4.9 Hz, 4H), 4.55 (s, 2H), 4.18 (t, J=5.8 Hz, 2H), 4.04 (t, J=5.9 Hz, 2H), 3.78 (s, 3H), 3.66 (s, 2H), 3.13 (s, 3H), 3.18-2.98 (m, 4H), 2.89 (q, J=13.4 Hz, 2H), 2.50 (d, J=17.4 Hz, 5H), 2.06 (d, J=13.7 Hz, 2H), 2.00-1.89 (m, 2H), 1.15 (dt, J=9.4, 7.5 Hz, 6H). LC-MS [ESI]: Calculated for C₅₂H₅₉N₁₅O₁₀S₂ [M+H⁺]: 1232.4, Found: 1232.4.

### Synthesis 2AL. Compound 38

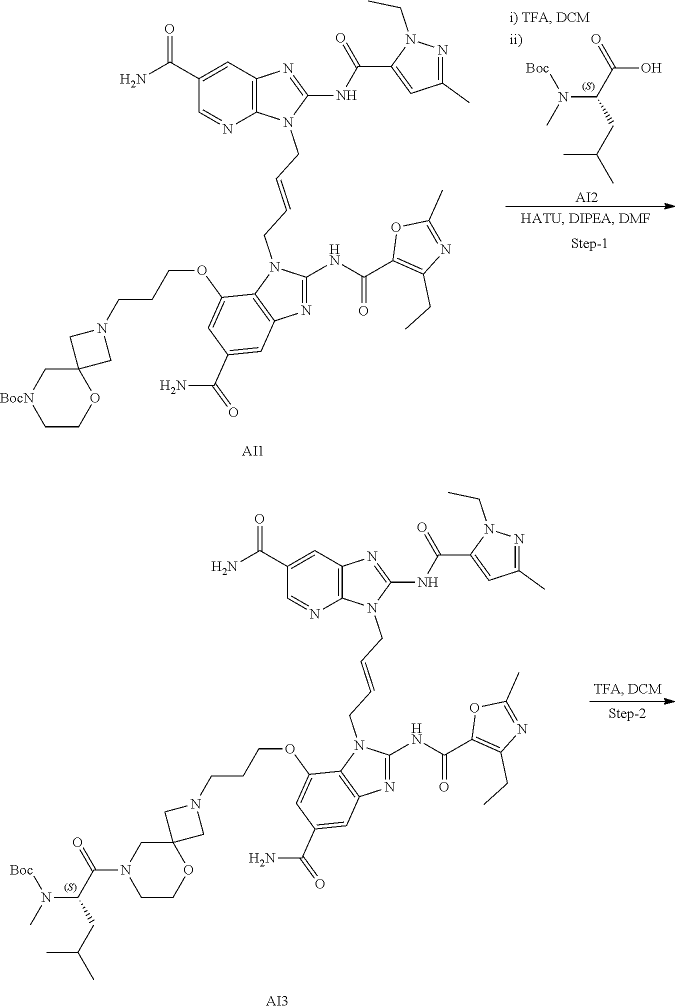

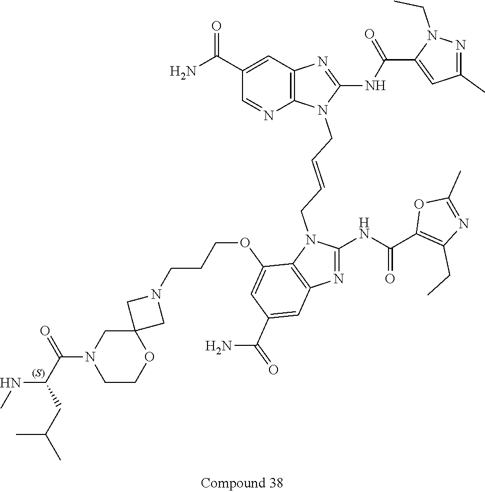

To a stirred solution of tert-butyl (E)-2-(3-((5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-2-(4-ethyl-2-methyloxazole-5-carboxamido)-1H-benzo[d]imidazol-7-yl)oxy)propyl)-5-oxa-2,8-diazaspiro[3.5]no(0.5 g, 0.519 mmol, 1 equiv.) in DCM (5 mL), Added TFA (2. mL), Stir rm 4 h at rt, Rm was Concentrated under reduced pressure to obtain the crude compound as a brown color Residue. (0.5 g). Crude compound was washed with Diethyl ether and used for the next step directly. To a stirred solution of crude (E)-N-(7-(3-(5-oxa-2,8-diazaspiro[3.5]nonan-2-yl)propoxy)-5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-1H-benzo[d]imidazol-2-yl)-4-ethyl-2-methyloxazole-5-carboxamide (0.2 g, 0.232 mmol, 1 equiv.) in DMF (2.000 mL) Charged EDC·HCl (0.066 g, 0.348 mmol, 1.5 equiv.) at 0° C., Added DIPEA (0.123 mL, 0.695 mmol, 3 equiv.) at 0° C., Added (tert-butoxycarbonyl)-L-leucine (0.085 g, 0.348 mmol, 1.5 equiv.) at 0° C. Maintained 12 h at rt, Reaction progress was monitored by TLC and LCMS. Rm charged into chilled water (10 mL) stir 2 h at rt, Filter the solid and solid slurry washed with chilled water. RM was washed with Acetonitrile and dried. Reaction was successful, product used for next step directly.

Synthesis of N-[5-carbamoyl-1-[(E)-4-[6-carbamoyl-2-[(2-ethyl-5-methyl-pyrazole-3-carbonyl)amino]imidazo[4,5-b]pyridin-3-yl]but-2-enyl]-7-[3-[8-[(2S)-4-methyl-2-(methylamino)pentanoyl]-5-oxa-2,8-diazaspiro[3.5]nonan-2-yl]propoxy]benzimidazol-2-yl]-4-ethyl-2-methyl-oxazole-5-carboxamide (Compound 38): To a stirred solution of crude tert-butyl (S,E)-(1-(2-(3-((5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-2-(4-ethyl-2-methyloxazole-5-carboxamido)-1H-benzo[d]imidazol-7-yl)oxy)propyl)-5-oxa-2,8-diazaspiro[3.5]nonan-8-yl)-4-methyl-1-oxopentan-2-yl)(methyl)carbamate (0.15 g, 0.139 mmol, 1 equiv.) in DCM (1.5 mL), Added TFA (0.75 mL) and stir rm 4 h at rt, Reaction progress was monitored by TLC and LCMS. Rm was Concentrated under reduced pressure to obtain the crude compound as a brown color Residue. (0.1 g). Crude compound was washed with Diethyl ether and purified by reverse phase preparative HPLC: C18 column, 10-90% acetonitrile in water with 0.1% TFA over 30 min. Desired product retention time 10 min to afford 13 mg of impure material. This material was repurified to afford N-[5-carbamoyl-1-[(E)-4-[6-carbamoyl-2-[(2-ethyl-5-methyl-pyrazole-3-carbonyl)amino]imidazo[4,5-b]pyridin-3-yl]but-2-enyl]-7-[3-[8-[(2S)-4-methyl-2-(methylamino)pentanoyl]-5-oxa-2,8-diazaspiro[3.5]nonan-2-yl]propoxy]benzimidazol-2-yl]-4-ethyl-2-methyl-oxazole-5-carboxamide (6 mg, 4.6%) over three steps as white solid. 1H NMR (400 MHz, DMSO) δ 12.88 (s, 1H), 12.75 (s, 1H), 10.07 (s, 1H), 8.90 (s, 2H), 8.69 (s, 1H), 8.13 (d, J=15.7 Hz, 2H), 7.95 (s, 1H), 7.65 (s, 1H), 7.56 (s, 1H), 7.36 (d, J=14.2 Hz, 2H), 6.51 (s, 1H), 5.90 (s, 1H), 5.74 (d, J=15.3 Hz, 1H), 4.92 (s, 2H), 4.78 (d, J=5.5 Hz, 2H), 4.49 (d, J=7.4 Hz, 2H), 4.44 (s, 1H), 4.14 (s, 4H), 4.02 (s, 3H), 3.78 (s, 1H), 2.78 (q, J=7.5 Hz, 2H), 2.38 (s, 3H), 2.10 (s, 3H), 1.94 (s, 2H), 1.62 (s, 1H), 1.57 (s, 2H), 1.25 (t, J=7.2 Hz, 3H), 1.01-0.83 (m, 9H). LC-MS [ESI]: Calculated for C₄₉H₆₃N₁₅O₈[M+H+]: 990.5, Found: 990.4.

### Synthesis 2AJ. Compound 66

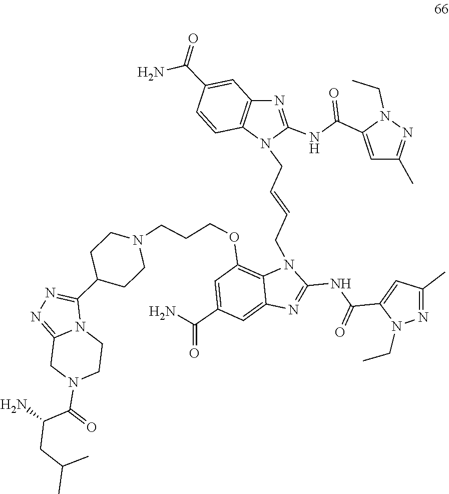 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)[C@@H](N)CC(C)C)C5)CC3)c21]

### Synthesis of (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazole-5-carboxamide Compound 66

Step 1. tert-butyl (S,E)-(1-(3-(1-(3-((5-carbamoyl-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate

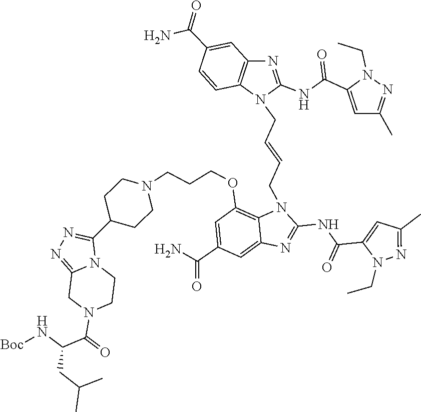 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)OC(C)(C)C)C5)CC3)c21]

To a mixture of (E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-(4-(5,6,7,8-tetrahydro-[1,2,4]triazolo[4,3-a]pyrazin-3-yl)piperidin-1-yl)propoxy)-1H-benzo[d]imidazole-5-carboxamide (89, 16.3 mg, 0.0173 mmol), (2S)-2-(tert-butoxycarbonylamino)-4-methyl-pentanoic acid (4.81 mg, 0.0208 mmol), DIEA (11.2 mg, 0.015 mL, 0.0867 mmol) in DMF (2 mL) was added HBTU (8.6 mg, 0.023 mmol). The reaction was stirred for 15 minutes. The reaction solution was purified by reverse phase preparative HPLC (acetonitrile/water+0.1% formic acid) to afford tert-butyl (S,E)-(1-(3-(1-(3-((5-carbamoyl-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate Boc protected 66 (10.9 mg, 55%). LCMS (ESI) m/z 1153.6 (M+H)

### Step 2. (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazole-5-carboxamide (66)

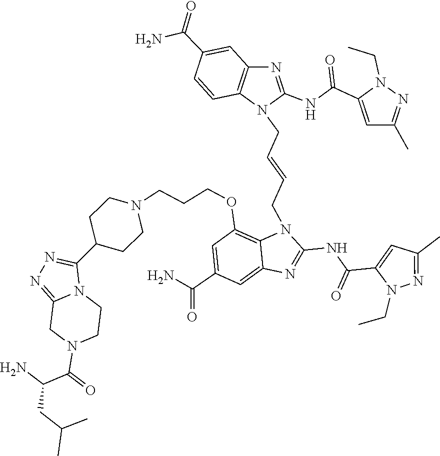 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)[C@@H](N)CC(C)C)C5)CC3)c21]

To a mixture of tert-butyl (S,E)-(1-(3-(1-(3-((5-carbamoyl-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (10.9 mg, 0.0095 mmol) in MeOH (2 mL) was added hydrogen chloride, 4.0 M in 1,4-dioxane (0.14 mL, 0.567 mmol). The reaction mixture was stirred at room temperature for 4 hours, then concentrated. The residue was dissolved in DMSO (2 mL) and 28-30% NH₃·H₂O (0.1 mL) then purified by reverse phase preparative HPLC (acetonitrile/water+0.1% 28-30% NH₃·H₂O) to afford (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazole-5-carboxamide (66), (white solid, 4.2 mg, 42%). LCMS (ESI) m/z 1053.6 (M+H). ¹H NMR (400 MHz, DMSO) δ 7.99 (s, 1H), 7.93 (s, 2H), 7.71 (d, J=8.4 Hz, 1H), 7.64 (s, 1H), 7.42 (d, J=8.2 Hz, 1H), 7.29 (s, 2H), 6.54 (d, J=10.3 Hz, 2H), 6.48 (s, 1H), 5.99 (s, 1H), 4.96 (s, 2H), 4.82 (s, 2H), 4.54 (dd, J=17.3, 7.4 Hz, 4H), 4.01 (s, 3H), 2.79 (s, 1H), 2.12 (d, J=12.5 Hz, 6H), 1.91 (s, 2H), 1.53 (s, 1H), 1.28 (dt, J=14.4, 7.1 Hz, 6H), 0.99-0.88 (m, 6H).

### Step 3. (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazole-5-carboxamide (66)

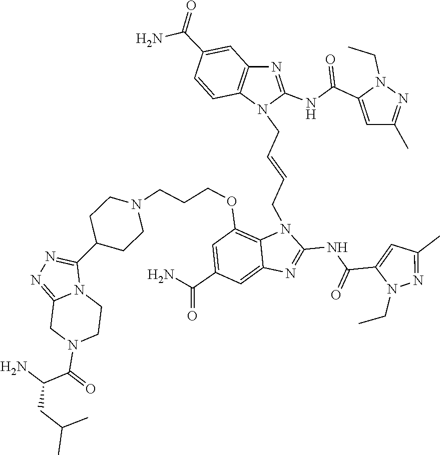 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)[C@@H](N)CC(C)C)C5)CC3)c21]

To a mixture of tert-butyl (S,E)-(1-(3-(1-(3-((5-carbamoyl-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (10.9 mg, 0.0095 mmol) in MeOH (2 mL) was added hydrogen chloride, 4.0 M in 1,4-dioxane (0.14 mL, 0.567 mmol). The reaction mixture was stirred at room temperature for 4 hours, then concentrated. The residue was dissolved in DMSO (2 mL) and 28-30% NH₃·H₂O (0.1 mL) then purified by reverse phase preparative HPLC (acetonitrile/water+0.1% 28-30% NH₃·H₂O) to afford (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazole-5-carboxamide (66), (white solid, 4.2 mg, 42%). LCMS (ESI) m/z 1053.6 (M+H). ¹H NMR (400 MHz, DMSO) δ 7.99 (s, 1H), 7.93 (s, 2H), 7.71 (d, J=8.4 Hz, 1H), 7.64 (s, 1H), 7.42 (d, J=8.2 Hz, 1H), 7.29 (s, 2H), 6.54 (d, J=10.3 Hz, 2H), 6.48 (s, 1H), 5.99 (s, 1H), 4.96 (s, 2H), 4.82 (s, 2H), 4.54 (dd, J=17.3, 7.4 Hz, 4H), 4.01 (s, 3H), 2.79 (s, 1H), 2.12 (d, J=12.5 Hz, 6H), 1.91 (s, 2H), 1.53 (s, 1H), 1.28 (dt, J=14.4, 7.1 Hz, 6H), 0.99-0.88 (m, 6H).

### Synthesis 2AK. Compound 90

### Synthesis of (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide 90

### Step 1. tert-butyl (S,E)-(1-(3-(1-(3-((5-carbamoyl-1-(4-(5-carbamoyl-7-methoxy-2-(3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate

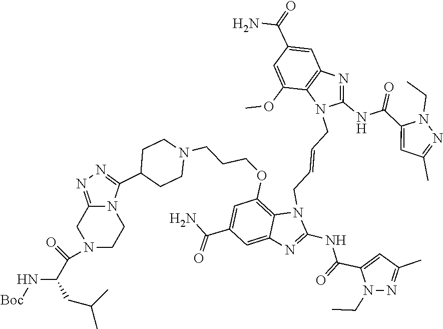 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)OC(C)(C)C)C5)CC3)c21]

To a mixture of 1-[(E)-4-[5-carbamoyl-2-[(2-ethyl-5-methyl-pyrazole-3-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]but-2-enyl]-2-[(2-ethyl-5-methyl-pyrazole-3-carbonyl)amino]-7-methoxy-benzimidazole-5-carboxamide; 2,2,2-trifluoroacetic acid (4, 17.1 mg, 0.014 mmol), (2S)-2-(tert-butoxycarbonylamino)-4-methyl-pentanoic acid (4.0 mg, 0.0171 mmol), DIEA (9.2 mg, 0.012 mL, 0.071 mmol) in DMF (2 mL) was added HBTU (7.1 mg, 0.0187 mmol). The reaction was stirred for 15 minutes. The reaction solution was purified by reverse phase preparative HPLC (acetonitrile/water+0.1% formic acid) to afford tert-butyl (S,E)-(1-(3-(1-(3-((5-carbamoyl-1-(4-(5-carbamoyl-7-methoxy-2-(3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (11.8 mg, 70%). LCMS (ESI) m/z 1183.6 (M+H).

### Step 2. (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide 90

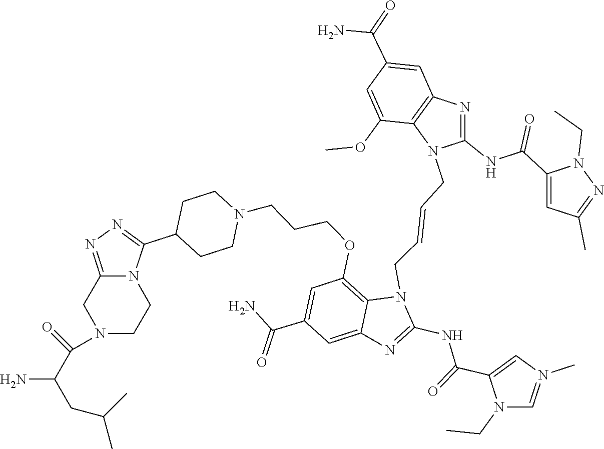

To a mixture of tert-butyl (S,E)-(1-(3-(1-(3-((5-carbamoyl-1-(4-(5-carbamoyl-7-methoxy-2-(3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (11.8 mg, 0.010 mmol) in MeOH (2 mL) was added hydrogen chloride, 4.0 M in 1,4-dioxane (0.15 mL, 0.60 mmol). The reaction mixture was stirred at room temperature for 4 hours, then concentrated. The residue was dissolved in DMSO (2 mL) and 28-30%0 NH₃·H₂O (0.1 mL) then purified by reverse phase preparative HPLC (acetonitrile/water+0.1% 28-30% NH₃·H₂O) to afford (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(i-ethyl-3-methyl-1H-pyrazole-5-carboxamido)-1H-benzo[d]imidazole-5-carboxamide 90 (white solid, 9.2 mg). LCMS (ESI) m/z 1083.6 (M+H). ¹H NMR (400 MHz, DMSO) δ 7.94 (s, 2H), 7.65 (d, J=6.5 Hz, 2H), 7.29 (d, J=14.9 Hz, 4H), 6.56 (s, 1H), 6.50 (d, J=2.8 Hz, 2H), 5.89-5.81 (m, 2H), 4.94 (s, 2H), 4.90 (s, 3H), 4.52 (dt, J=21.2, 7.2 Hz, 3H), 4.01 (s, 2H), 3.95 (s, 5H), 3.72 (s, 4H), 2.76 (s, 2H), 2.29 (s, 4H), 2.11 (d, J=17.6 Hz, 7H), 1.80 (d, J=11.3 Hz, 4H), 1.72 (s, 3H), 1.67 (s, 6H), 1.28 (dt, J=17.9, 7.1 Hz, 8H), 0.91 (d, J 6.6 Hz, 6H).

### Example 3. Synthesis of Select Linker-Payloads

### Synthesis 3A. LP-1-1

LP-1-1 was synthesized as described in Scheme 18.

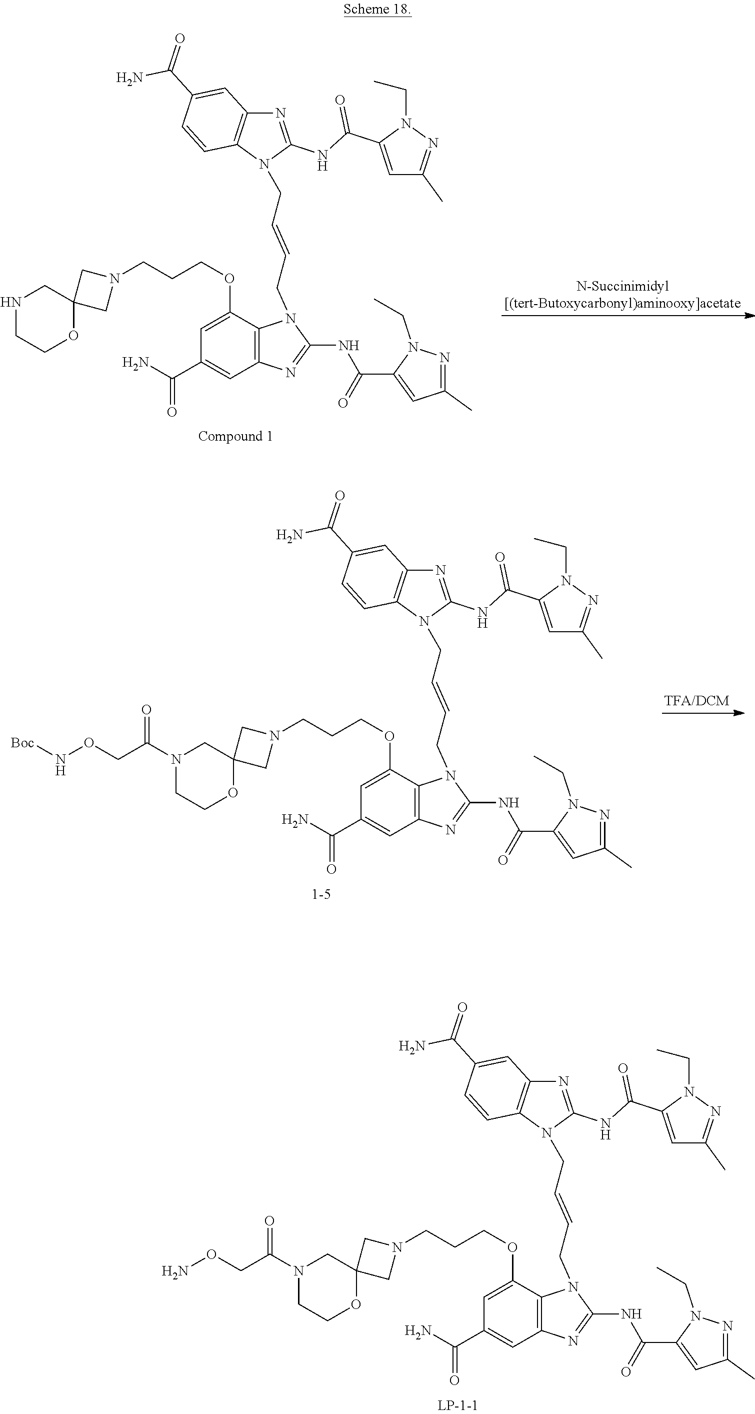 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(CNCCO4)C3)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)CONC(=O)OC(C)(C)C)CCO4)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)CON)CCO4)c21]

Compound 1-5: Compound 1 (80 mg) was dissolved in anhydrous DMF (4 mL). To this solution, N-succinimidyl[(tert-butoxycarbonyl)aminooxy]acetate (TCI, 29 mg, 0.1 mmol) was added, followed by DIPEA (0.4 mmol, 0.07 mL), and the mixture was stirred at room temperature for 1 hour. The crude mixture was purified directly by reverse phase preparative HPLC to give compound I-5 as a white powder after lyophilization (71 mg); LC-MS [ESI]: Calculated for C₅₀H₆₃N₁₅O₁₀ [M+H+]: 1034.49, Found: 1034.6.

LP-1-1: Compound 1-5 (70 mg) was treated with TFA/DCM (1/4, v/v, 2 mL) at room temperature for 10 minutes. The reaction mixture was evaporated to dryness under reduced pressure and the residue was purified by reverse phase preparative HPLC to give LP-1-1 as a white powder (40 mg, TFA salt); LC-MS [ESI]: Calculated for C₄₅H₅₅N₁₅O₈ [M+H+]: 934.44, Found: 934.6; ¹H NMR (400 MHz, DMSO) δ 12.88 (s, 2H), 10.18 (d, J=161.4 Hz, 3H), 8.13-7.89 (m, 3H), 7.80-7.61 (m, 2H), 7.37 (dd, J=20.5, 10.6 Hz, 4H), 6.53 (d, J=9.4 Hz, 2H), 5.97 (d, J=15.5 Hz, 1H), 5.69 (d, J=15.8 Hz, 2H), 4.95 (d, J=5.3 Hz, 2H), 4.82 (d, J=5.6 Hz, 2H), 4.76-4.37 (m, 7H), 4.08 (s, 7H), 3.98-3.50 (m, 29H), 3.37 (d, J=57.1 Hz, 8H), 2.13 (s, 6H), 1.84 (s, 2H), 1.28 (td, J=7.1, 3.3 Hz, 6H).

### Synthesis 3B. LP-6-1, LP-10-1, LP-2-1, and LP-58-1

LP-6-1, LP1-10-1, LP-2-1, and LP-58-1 were synthesized in an analogous fashion using the same scheme and methods as described herein. The final compounds were purified by reverse phase preparative HPLC and characterized by ¹HNMR, LCMS data.

| Linker | |
|---|---|
| Payload | Structure |
| No. | ¹HNMR, LCMS |
| LP-6-1 | 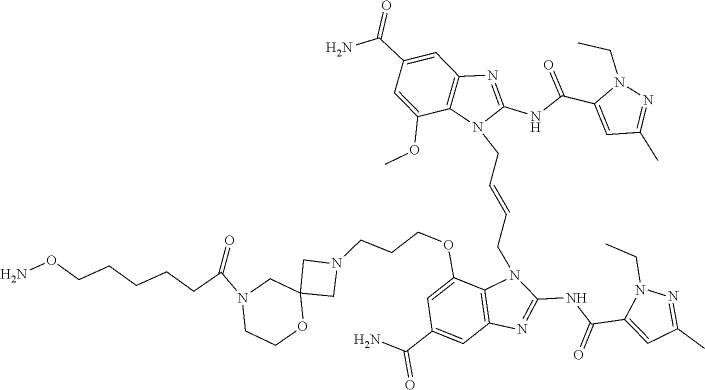 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(C3)CN(C(=O)CCCCCON)CCO4)c21] ¹H NMR (400 MHz, DMSO) δ 12.87 (s, 2H), 10.43 (s, 3H), 7.97 (s, 2H), 7.67 (d, J = 9.4 Hz, 2H), 7.45-7.18 (m, 3H), 6.53 (s, 2H), 5.79 (q, J = 16.2 Hz, 2H), 4.92 (dd, J = 21.2, 5.1 Hz, 3H), 4.53 (d, J = 7.4 Hz, 4H), 4.09 (d, J = 60.3 Hz, 4H), 3.96-3.77 (m, 4H), 3.77-3.60 (m, 5H), 3.56 (s, 1H), 3.41 (s, 4H), 2.35 (s, 2H), 2.12 (d, J = 3.4 Hz, 5H), 1.81 (s, 2H), 1.55 (d, J = 37.9 Hz, 3H), 1.28 (td, J = 7.1, 2.7 Hz, 6H). ); LC-MS [ESI]: Calculated for C₅₀H₆₆N₁₅O₉ [M + H⁺]: 1020.51, Found: 1020.53. |
| LP-10-1 | 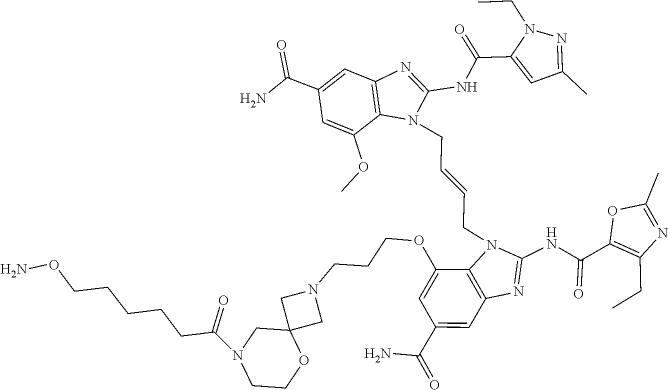 [SMILES: CCc1nc(C)oc1C(=O)Nc1nc2cc(C(N)=O)cc(OCCCN3CC4(C3)CN(C(=O)CCCCCON)CCO4)c2n1C/C=C/Cn1c(NC(=O)c2cc(C)nn2CC)nc2cc(C(N)=O)cc(OC)c21] LC-MS [ESI]: Calculated for C₅₀H₆₄N₁₄O₁₀ [M + H⁺]: 1021.49, Found: 1021.5. |
| LP-2-1 | 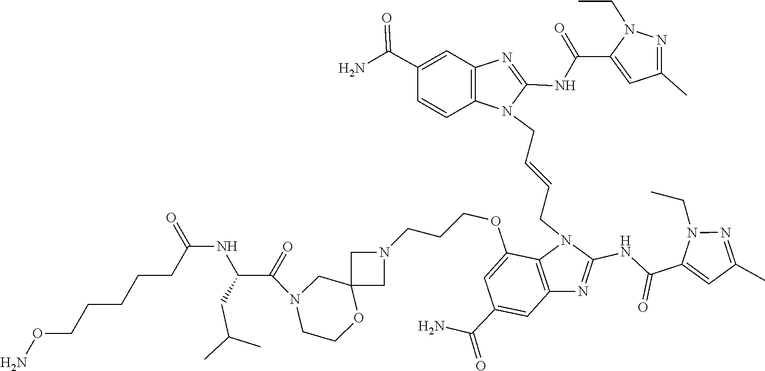 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@H](CC(C)C)NC(=O)CCCCCON)CCO4)c21] ¹H NMR (400 MHz, DMSO) δ 10.41 (s, 4H), 8.12 (d, J = 8.1 Hz, 1H), 7.96 (d, J = 13.6 Hz, 3H), 7.73-7.63 (m, 2H), 7.38 (d, J = 8.4 Hz, 3H), 7.32 (s, 1H), 6.53 (d, J = 12.7 Hz, 2H), 5.97 (d, J = 15.9 Hz, 1H), 5.69 (d, J = 15.8 Hz, 1H), 4.95 (s, 2H), 4.81 (d, J = 5.5 Hz, 2H), 4.69 (s, 1H), 4.52 (d, J = 7.3 Hz, 4H), 4.06 (s, 3H),3.96 (s, 4H), 3.89 (d, J = 6.5 Hz, 1H), 3.86 (s, 6H), 3.67 (s, 1H), 3.50 (s, 2H), 3.04 (s, 1H), 2.12 (s, 5H), 2.05 (dt, J = 14.2, 7.3 Hz, 1H), 1.81 (s, 2H), 1.58-1.44 (m,5H), 1.27 (td, J = 7.1, 2.3 Hz, 8H), 0.86 (t, J = 6.6 Hz, 6H). LC-MS [ESI]: Calculated for C₅₅H₇₅N₁₆O₉ [M + H⁺]: 1103.58, Found: 1103.6. |
| LP-58-1 | 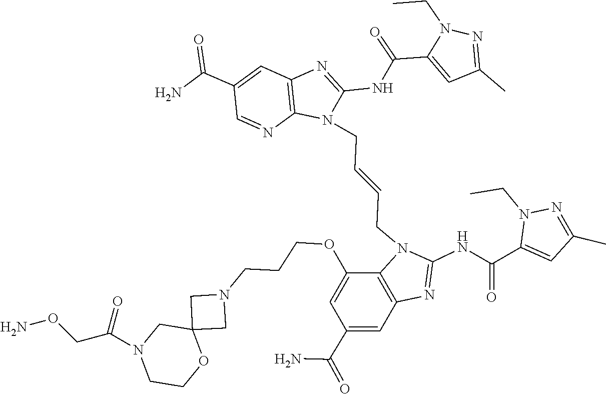 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CC4(C3)CN(C(=O)CON)CCO4)c21] |

### Synthesis 3C. LP-1-2 (Aminooxy-PEG4-vcpAB-Compound 1)

LP-1-2 was synthesized as described in Scheme 19.

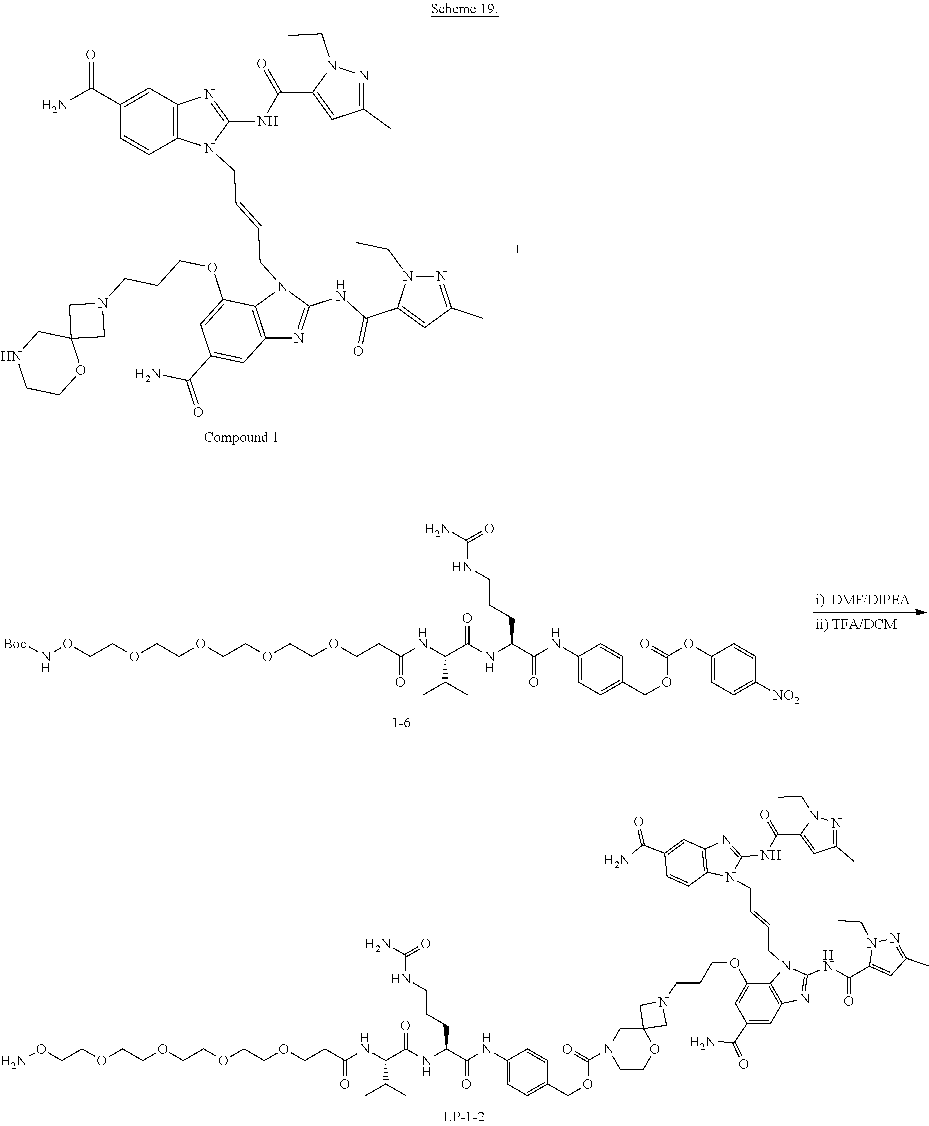 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(CNCCO4)C3)c21 ; CC(C)[C@H](NC(=O)CCOCCOCCOCCOCCONC(=O)OC(C)(C)C)C(=O)N[C@@H](CCCNC(N)=O)C(=O)Nc1ccc(COC(=O)Oc2ccc([N+](=O)[O-])cc2)cc1 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)OCc3ccc(NC(=O)[C@H](CCCNC(N)=O)NC(=O)[C@@H](NC(=O)CCOCCOCCOCCOCCON)C(C)C)cc3)CCO4)c21]

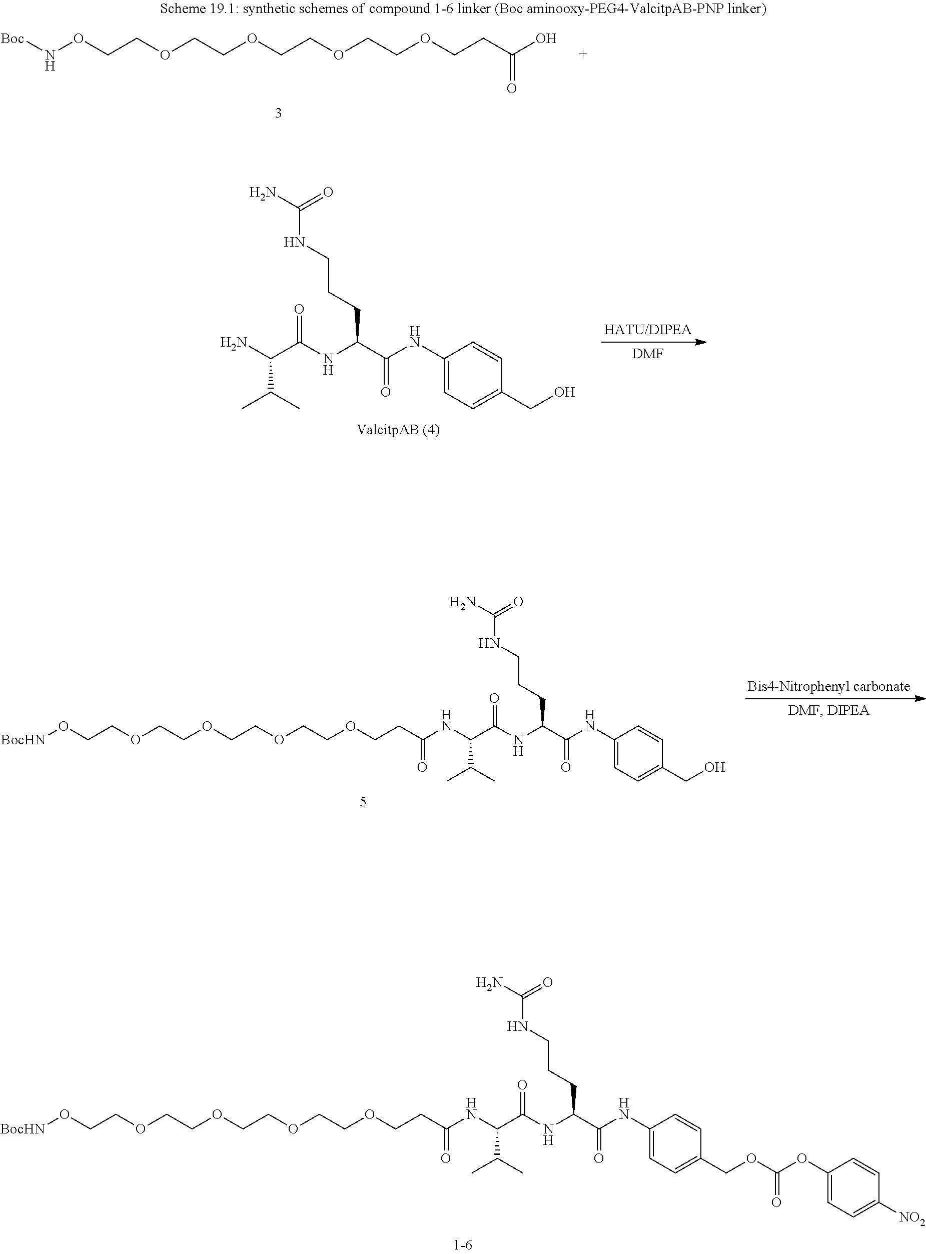

### Synthesis of ValcitpAB (Compound 4)

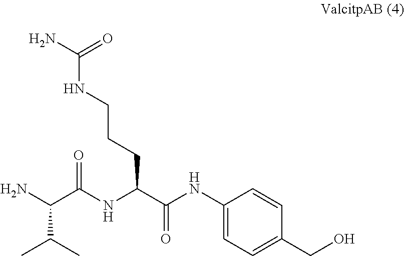 [SMILES: CC(C)[C@H](N)C(=O)N[C@@H](CCCNC(N)=O)C(=O)Nc1ccc(CO)cc1]

To an oven dried 100 mL RB flask was equipped with magnetic stir bar, added Fmoc-Val-cit-pAB (490 mg, 0.8 mmol) in NMP (8 mL), this mixture was treated with diethylamine (1.6 mL) at rt. The reaction was stirred at rt for 16 h. The thick slurry was treated with DCM (40 mL), and the solids were collected filtration, washed with DCM and dried in vacuum (280 mg). LCMS 380.5 (M+H).

### Synthesis of compound 5

To an oven dried 250 mL flask was equipped with magnetic stir bar, added Boc-Aminooxy-PEG4-acid (3) (2.5 g, 6.55 mmol), anhydrous DMF (25 mL), the clear solution was cooled to 0° C. HATU (2.73 g, 7.20 mmol), ValcitpAB (compound 4, 2.76 g, 7.20 mmol) & diisopropylethylamine (3.4 mL, 19.65 mmol) were sequentially added to the Boc-aminooxy-PEG4-acid compound 3 in DMF, the reaction slurry was flushed with argon and then was stirred at rt for 1h under N2 atm. LC-MS showed completion of the reaction. DMF Solvent was partially removed and the crude compound was purified by reverse phase preparative HPLC (method details are below) to obtain compound 5 (3.01 g) as a white solid. LCMS 743.8 (M+H).

Synthesis of compound I-6: To an oven dried 100 mL flask was equipped with magnetic stir bar, added compound (5) (3.01 g, 4.06 mmol), anhydrous DMF (25 mL) and the clear solution was flushed with argon. After which, bis PNP carbonate (1.48 g, 4.86 mmol) and DIPEA (2 mL) were sequentially added at rt to the above solution. Reaction was stirred at rt for 4 h under N₂ atm, LCMS showed completion of the reaction. DMF Solvent was partially removed and the crude compound was purified by reverse phase preparative HPLC to obtain compound I-6 (2.4 g) as an off white solid. LCMS 908.4 (M+H).

Compound 1 (60 mg) was dissolved in anhydrous DMF (3 mL). To this solution, Boc-NO-PEG4-VC-PAB-PNP (1-6, 58 mg) was added, followed by DIPEA (0.4 mmol, 0.07 mL), and the mixture was stirred at room temperature for 3 hours. LCMS showed the reaction was completed. The crude mixture was directly purified by reverse phase preparative HPLC to give Boc-protected LP-1-2s a white powder after lyophilization (82 mg). LC-MS [ESI]: Calculated for C₇₈H₁₀₈N₂₀O₁₉ [M+H⁺]: 1629.81, Found: 1629.9.

Compound 1-7 (82 mg) was treated with TFA/DCM (1/4, v/v, 2 mL) at room temperature for 15 minutes. After which, LC-MS showed the desired product formation, The reaction mixture was evaporated to dryness under reduced pressure and the residue was purified by reverse phase preparative HPLC to give LP-1-2 as a white powder (61 mg, TFA salt). ¹H NMR (400 MHz, DMSO) δ 12.84 (s, 2H), 10.02 (s, 2H), 8.14 (d, J=7.4 Hz, 1H), 8.01-7.91 (m, 3H), 7.87 (d, J=8.7 Hz, 1H), 7.74-7.64 (m, 2H), 7.60 (d, J=8.5 Hz, 2H), 7.44-7.27 (m, 5H), 6.54 (d, J=9.0 Hz, 2H), 5.97 (dd, J=20.7, 5.5 Hz, 3H), 5.70 (d, J=15.2 Hz, 2H), 5.43 (s, 2H), 5.05 (s, 2H), 5.02-4.90 (m, 2H), 4.82 (d, J=5.6 Hz, 3H), 4.53 (d, J=7.0 Hz, 4H), 4.38 (q, J=7.3 Hz, 1H), 4.24 (dd, J=8.7, 6.7 Hz, 2H), 4.18-3.83 (m, 9H), 3.79-3.23 (m, 72H), 2.99 (dp, J=25.6, 6.3 Hz, 4H), 2.68-2.28 (m, 22H), 2.12 (s, 5H), 1.96 (h, J=6.8 Hz, 1H), 1.81 (s, 2H), 1.68 (s, 3H), 1.28 (td, J=7.1, 4.9 Hz, 6H), 0.84 (dd, J=12.3, 6.7 Hz, 6H). LC-MS [ESI]: Calculated for C₇₃H₁₀₀N₂₀O₁₇ [M+H⁺]: 1529.76, Found: 1529.9.

### Synthesis 3D. LP-1-3 (Aminooxy-β-Glucuronidase Cleavable Compound 1)

LP-1-3 was synthesized as described in Scheme 20.

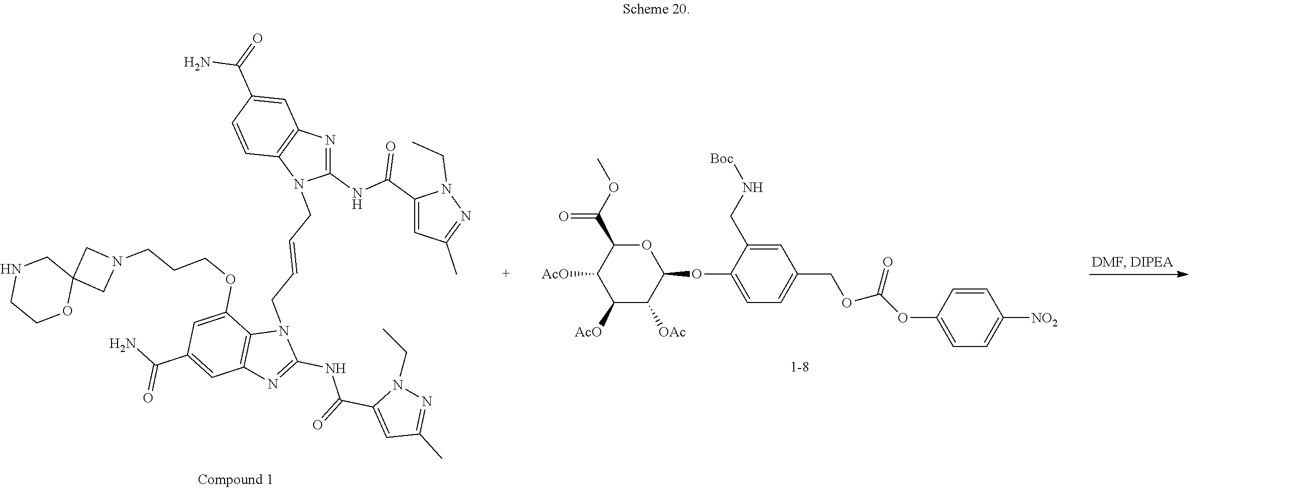 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(CNCCO4)C3)c21 ; COC(=O)[C@H]1O[C@@H](Oc2ccc(COC(=O)Oc3ccc([N+](=O)[O-])cc3)cc2CNC(=O)OC(C)(C)C)[C@H](OC(C)=O)[C@@H](OC(C)=O)[C@@H]1OC(C)=O]

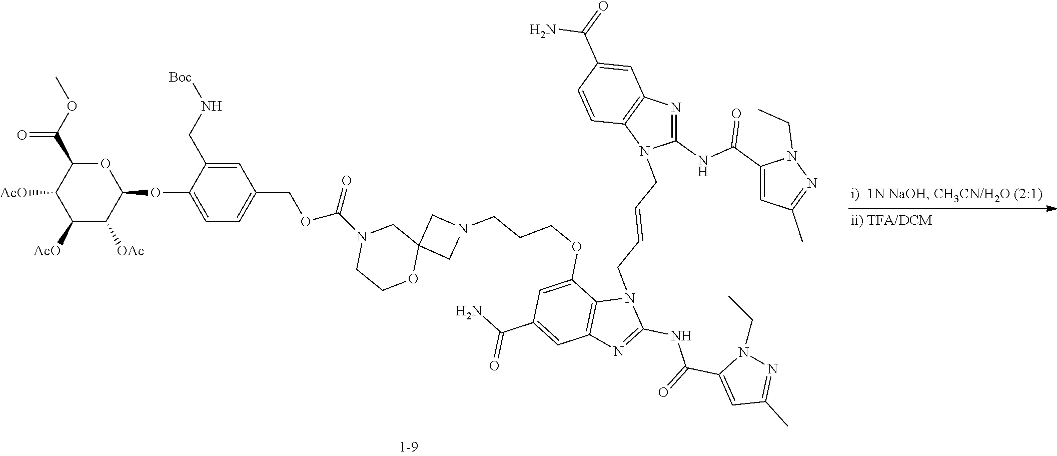 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)OCc3ccc(O[C@@H]5O[C@H](C(=O)OC)[C@@H](OC(C)=O)[C@H](OC(C)=O)[C@H]5OC(C)=O)c(CNC(=O)OC(C)(C)C)c3)CCO4)c21]

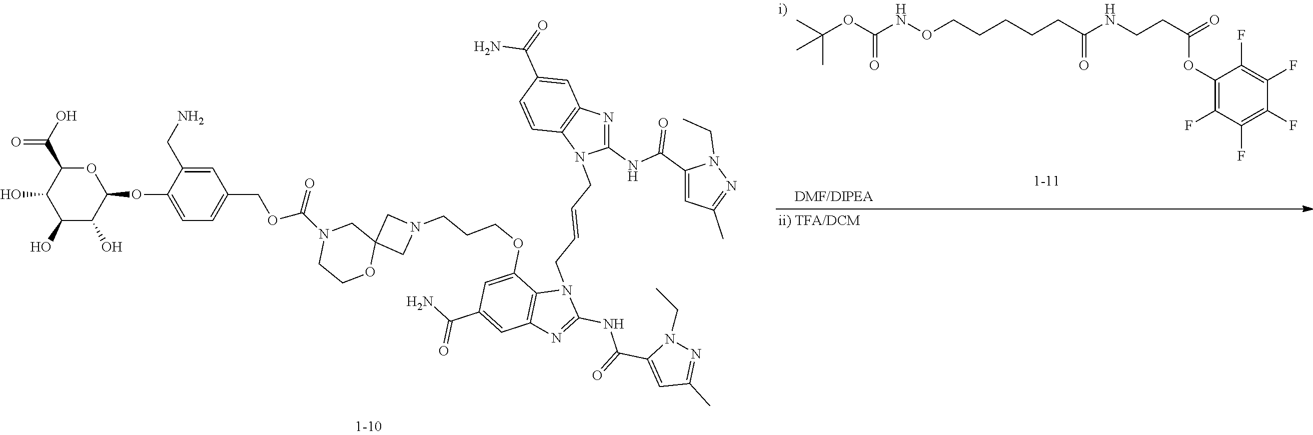 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)OCc3ccc(O[C@@H]5O[C@H](C(=O)O)[C@@H](O)[C@H](O)[C@H]5O)c(CN)c3)CCO4)c21 ; CC(C)(C)OC(=O)NOCCCCCC(=O)NCCC(=O)Oc1c(F)c(F)c(F)c(F)c1F]

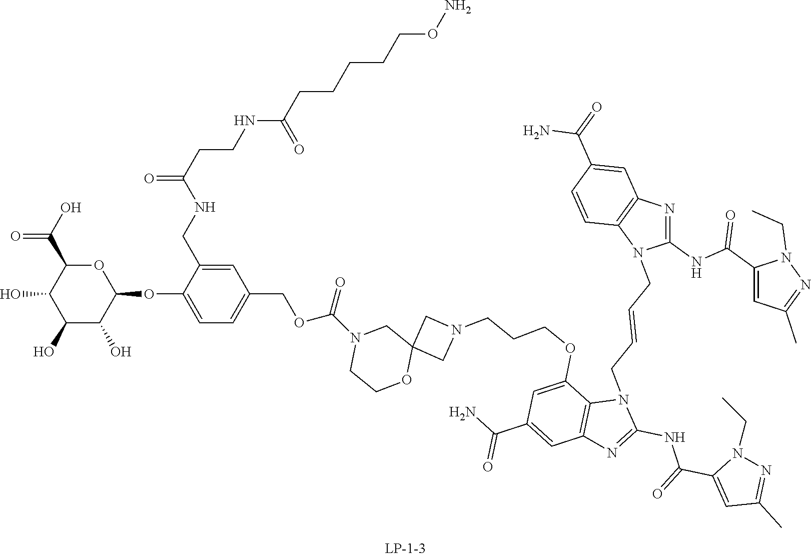 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)OCc3ccc(O[C@@H]5O[C@H](C(=O)O)[C@@H](O)[C@H](O)[C@H]5O)c(CNC(=O)CCNC(=O)CCCCCON)c3)CCO4)c21]

Compound (2S,3R,4S,5S,6S)-2-(2-(((tert-butoxycarbonyl)amino)methyl)-4-(((2-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)-5-oxa-2,8-diazaspiro[3.5]nonane-8-carbonyl)oxy)methyl)phenoxy)-6-(methoxycarbonyl)tetrahydro-2H-pyran-3,4,5-triyl triacetate 1-9: To a solution of b-Glu-PNP compound I-8 (46 mg, 63 μmol), which was synthesized as described in WO 2024/006542, and Compound 1 (TFA salt, 60 mg) in DMF (2 mL) was added DIPEA (44 μL). The reaction mixture was stirred at room temperature (22° C.) for 5 hours. LCMS showed the desired product formation. The crude compound was directly by reverse phase preparative HPLC to give compound I-9 as a white powder after lyophilization (84 mg). LC-MS [ESI]: Calculated for C₇₀H₈₅N₁₅O₂₀ [M+H⁺]: 1456.61, Found: 1456.7.

(2S,3S,4S,5R,6S)-6-(2-(aminomethyl)-4-(((2-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)-5-oxa-2,8-diazaspiro[3.5]nonane-8-carbonyl)oxy)methyl)phenoxy)-3,4,5-trihydroxytetrahydro-2H-pyran-2-carboxylic acid (Compound 1-10): Compound 1-9 (84 mg) was dissolved in acetonitrile/water (2/1, 4 mL). To this mixture, 1N aq. NaOH (0.45 mL) was added, and the reaction was stirred at room temperature. After 3 hours, 0.3 mL of 1N hydrochloric acid was added and the mixture was evaporated to dryness under reduced pressure. The residue obtained was treated with TFA/DCM (1/4, v/v, 3 mL) at room temperature for 20 minutes. The mixture was diluted with toluene (30 mL) and then evaporated to dryness under reduced pressure to give the crude compound I-10 as a TFA salt. LC-MS [ESI]: Calculated for C₅₈H₆₉N₁₅O₁₅ [M+H⁺]: 1216.51, Found: 1216.6.

LP-1-3: The above prepared compound I-10 was dissolved in anhydrous DMF (4 mL) and Intermediate 1-11 (24 mg, 0.05 umol) was added, followed by DIEA (0.1 mL). The reaction mixture was stirred at room temperature for 30 minutes and purified directly by reverse phase preparative HPLC to give Boc-protected LP-3-1 as a white powder (48 mg). Boc-protected LP-1-3 (48 mg) was treated with TFA/DCM (1/4, v/v, 2 mL) at room temperature for 20 minutes and the mixture was purified directly by reverse phase preparative HPLC to give LP-1-3 as a white powder (43 mg); ¹H NMR (400 MHz, DMSO) δ 12.86 (s, 2H), 8.20 (t, J=6.0 Hz, 1H), 8.01-7.92 (m, 2H), 7.82 (t, J=5.7 Hz, 1H), 7.73-7.64 (m, 2H), 7.42-7.30 (m, 3H), 7.25 (dd, J=8.5, 2.2 Hz, 1H), 7.18 (d, J=2.2 Hz, 1H), 7.07 (d, J=8.5 Hz, 1H), 6.54 (d, J=9.4 Hz, 2H), 5.97 (dt, J=15.6, 5.4 Hz, 1H), 5.69 (d, J=15.6 Hz, 1H), 5.04 (s, 2H), 4.96 (dd, J=8.4, 4.5 Hz, 3H), 4.82 (d, J=5.6 Hz, 2H), 4.53 (d, J=7.5 Hz, 4H), 4.29 (qd, J=15.5, 5.8 Hz, 3H), 4.06 (s, 4H), 3.96-3.85 (m, 5H), 3.47-3.39 (m, 4H), 3.39-3.31 (m, 4H), 3.26 (dt, J=13.2, 7.7 Hz, 4H), 2.36-2.25 (m, 2H), 2.12 (s, 5H), 2.04 (t, J=7.4 Hz, 2H), 1.82 (s, 2H), 1.50 (dq, J=29.4, 7.4 Hz, 4H), 1.28 (td, J=7.1, 4.5 Hz, 7H). LC-MS [ESI]: Calculated for C67H85N17O18 [M+H⁺]: 1416.63, Found: 1416.7.

### Synthesis of Intermediate 1-11

Intermediate 1-11 was prepared following Scheme 21.

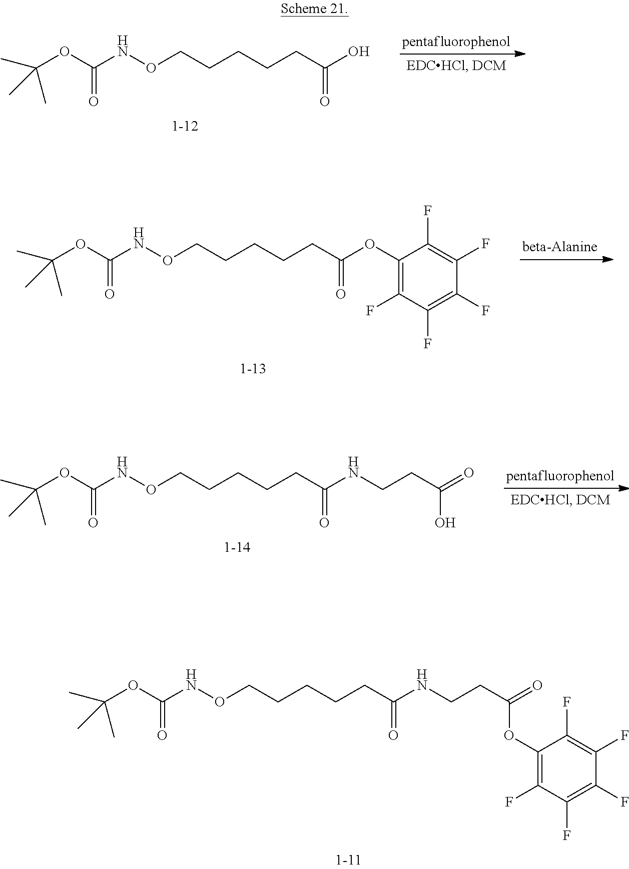 [SMILES: CC(C)(C)OC(=O)NOCCCCCC(=O)O ; CC(C)(C)OC(=O)NOCCCCCC(=O)Oc1c(F)c(F)c(F)c(F)c1F ; CC(C)(C)OC(=O)NOCCCCCC(=O)NCCC(=O)O ; CC(C)(C)OC(=O)NOCCCCCC(=O)NCCC(=O)Oc1c(F)c(F)c(F)c(F)c1F]

Compound 1-14: To a solution of compound I-12 (247 mg, 1 mmol) in anhydrous DCM (5 mL) was added pentafluorophenol (368 mg, 2 mmol) and EDC·HCl (383 mg, 2 mmol). The mixture was stirred at room temperature for 1 hour and then diluted with DCM (50 mL), and washed with 1N hydrochloric acid (30 mL) and water (30 mL). The organic layer was dried and concentrated under reduced pressure to give the crude compound I-13, which was dissolved in acetonitrile (5 mL). To this solution, a mixture of beta-alanine (180 mg) in water (4 mL) was added, followed by DIEA (0.7 mL, 4 mmol). After 15 minutes, the reaction was diluted with EtOAc (100 mL), and then HCl (1N, aq., 20 mL) was added. The organic layer was collected, washed with water (20 mL), dried, and concentrated under reduced pressure to give the crude product 1-14.

Compound 1-11: The crude compound I-14 was dissolved in DCM (5 mL). To this solution, pentafluorophenol (368 mg, 2 mmol) and EDC·HCl (383 mg, 2 mmol) were added. The mixture was stirred at room temperature for 1 hour and then diluted with DCM (50 mL), washed with 1N hydrochloric acid (30 mL) and water (30 mL). The organic layer was dried and concentrated under reduced pressure to give the crude compound I-11 which was purified by reverse phase preparative HPLC to give the pure product 1-11 as a viscous syrup (310 mg). LC-MS [ESI]: Calculated for C₂₀H₂₆F₅N₂O₆ [M+H⁺]: 485.16, Found: 485.2.

### Synthesis 3E. LP-2-2 (Aminooxy-PEG4-AAN-Compound 2)

LP-2-2 was synthesized as described in Scheme 22.

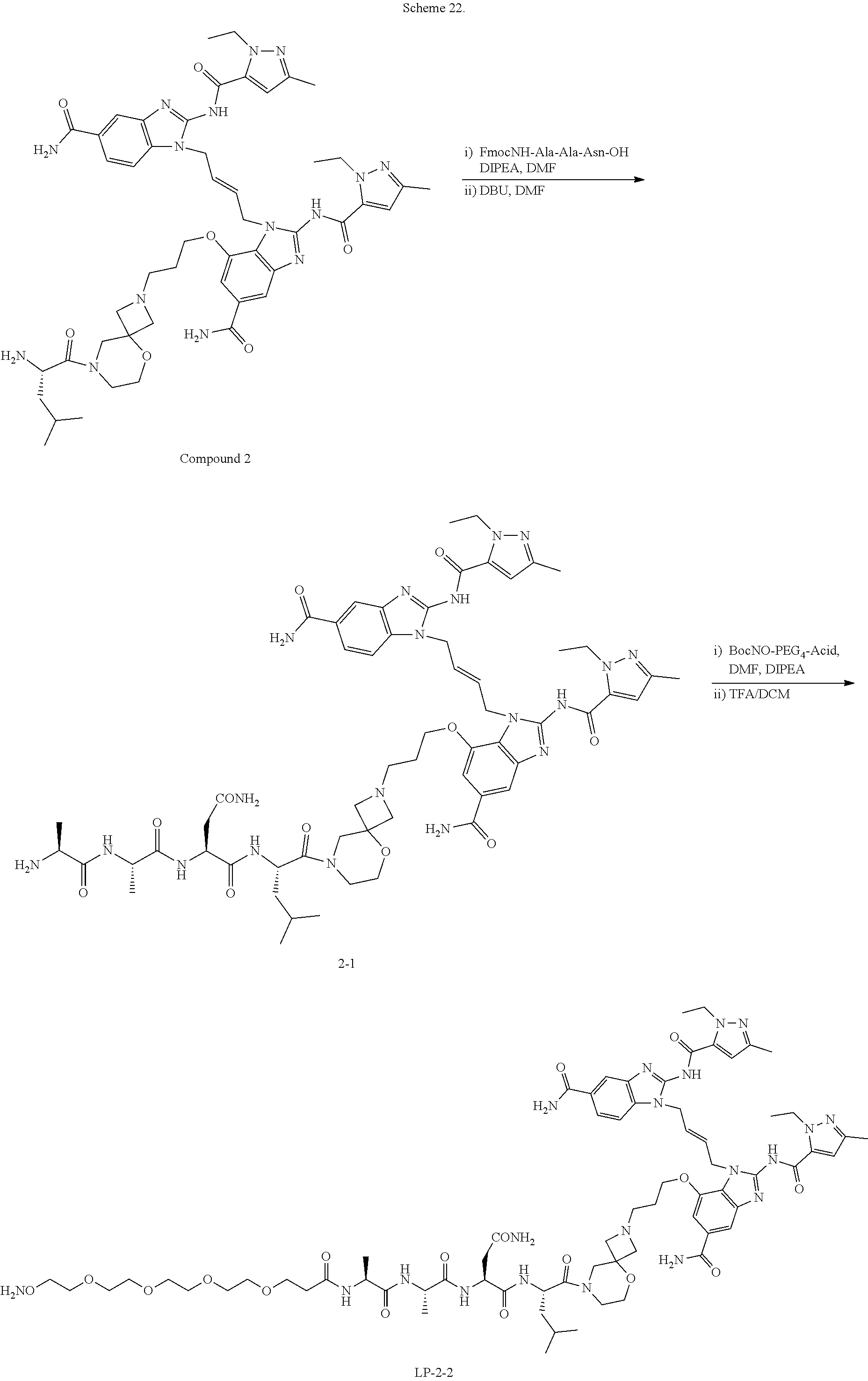

Compound 2-1: To a solution of Compound 2 (TFA salt, 24 mg, 20 mmol) and Fmoc-Ala-Ala-Asn-OH (11 mg, 22 μmol) in DMF (1 mL) was added PyAOP (11 mg, 21 μmol), followed by DIPEA (14 μL). After 10 minutes, DBU (40 mL) was added, and the mixture was stirred at room temperature for 15 minutes. The crude reaction was purified directly by reverse phase preparative HPLC to give compound 2-1 (22 mg, TFA salt) as a white powder. LC-MS [ESI]: Calculated for C₅₉H₇₉N₁₉O₁₁ [M+H⁺]: 1230.62, Found: 1230.7.

LP-2-2: To a solution of compound 2-1 (TFA salt, 22 mg, 15 μmol) and BocNO-PEG4-COOH (6 mg, 16 μmol) in DMF (1 mL) was added PyAOP (9 mg, 16 μmol), followed by DIPEA (11 μL). After 30 minutes, the crude reaction mixture was purified directly by reverse phase preparative HPLC to give compound to give Boc-protected LP-2-2 (18 mg, TFA salt) as a white powder.

Boc-protected LP-2-2 (18 mg) was treated with TFA/DCM (1/4, v/v, 1 mL) at room temperature for 20 mins and the mixture was purified directly by reverse phase preparative HPLC to give LP-2-2 as a white powder (14 mg); ¹H NMR (400 MHz, DMSO) δ 12.86 (s, 3H), 10.27 (s, 5H), 8.15-7.91 (m, 5H), 7.76 (d, J=8.2 Hz, 1H), 7.66 (d, J=5.5 Hz, 2H), 7.45-7.25 (m, 5H), 6.91 (s, 1H), 6.52 (s, 2H), 5.92-5.67 (m, 2H), 4.91 (dd, J=14.0, 4.2 Hz, 4H), 4.67 (s, 2H), 4.52 (q, J=6.9 Hz, 5H), 4.22 (dt, J=22.2, 7.0 Hz, 3H), 4.12-3.80 (m, 1OH), 3.81-3.33 (m, 40H), 2.47-2.27 (m, 4H), 1.79 (s, 2H), 1.70-1.33 (m, 4H), 1.34-1.06 (m, 11H), 0.85 (d, J=6.4 Hz, 6H). LC-MS [ESI]: Calculated for (C₇₀H₁₀₀N₂₀O₁₇=[M+H⁺]: 1493.76, Found: 1493.9.

### Synthesis 3F. LP-16-1

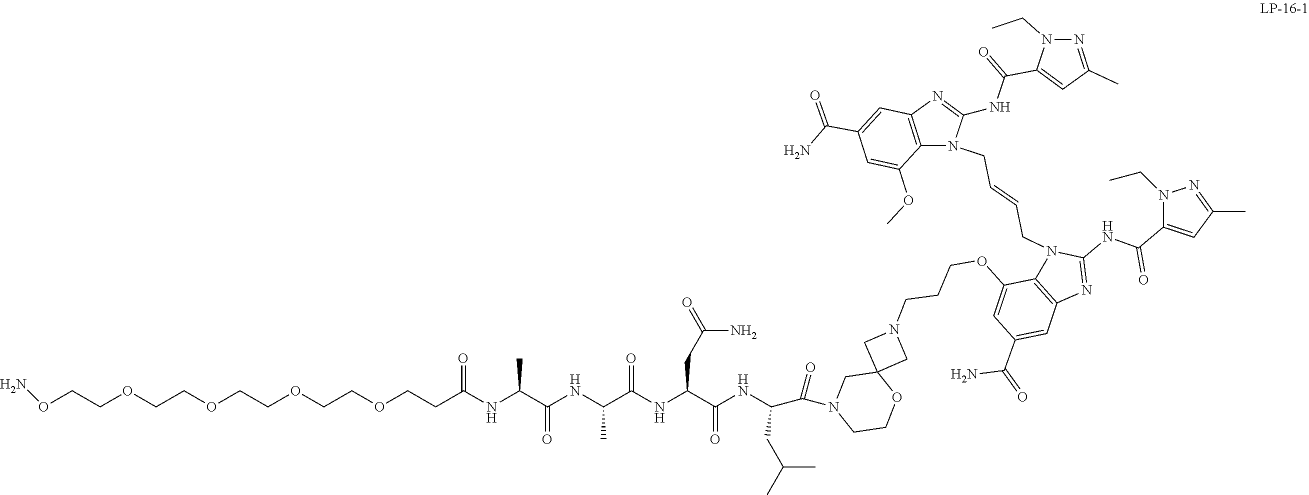 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(C3)CN(C(=O)[C@H](CC(C)C)NC(=O)[C@H](CC(N)=O)NC(=O)[C@H](C)NC(=O)[C@H](C)NC(=O)CCOCCOCCOCCOCCON)CCO4)c21]

LP-16-1 was synthesized using the same as described above starting from Compound 16. LC-MS [ESI]: Calculated for C₇₁H₁₀₂N₂₀O₁₈ [M+H⁺]: 1523.77, Found: 1523.9.

### Synthesis 3G

The following linker payloads were synthesized using the methods described here from Compound 4. The final compounds are purified by reverse phase preparative HPLC.

| LP | Structure |
|---|---|
| Number | ¹H NMR and LC-MS |
| LP-4-1 | 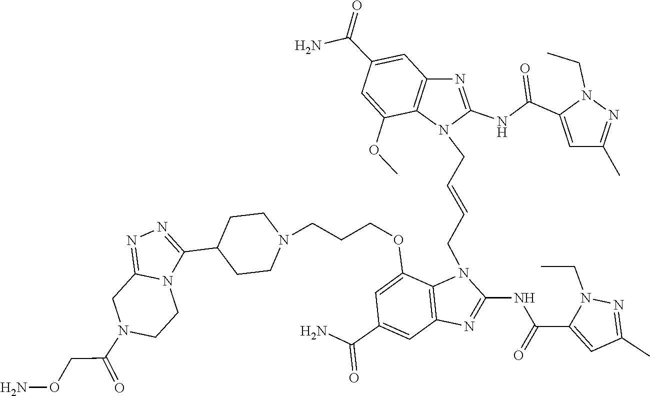 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21] ¹H NMR (400 MHz, DMSO) δ 9.35 (s, 2H), 8.01 (d, J = 25.2 Hz, 3H), 7.68 (d, J = 6.6 Hz, 3H), 7.55-7.13 (m, 5H), 6.72-6.26 (m, 3H), 6.00-5.62 (m, 3H), 4.92 (dd, J = 12.7, 4.8 Hz, 5H), 4.78 (d, J = 28.9 Hz, 5H), 4.53 (dt, J = 14.1, 7.2 Hz, 6H), 4.10-3.93 (m, 8H), 3.88 (s, 3H), 3.73 (d, J = 8.8 Hz, 5H), 3.53-3.37 (m, 3H), 2.93 (d, J = 38.2 Hz, 3H), 2.18-2.02 (m, 8H), 1.93 (d, J = 12.6 Hz, 4H), 1.29 (dt, J = 14.4, 7.1 Hz, 6H). LC-MS [ESI]: Calculated for C₅₀H₆₃N₁₈O₈ [M + H⁺]: 1043.5, Found: 1043.5. |
| LP-4-2 | 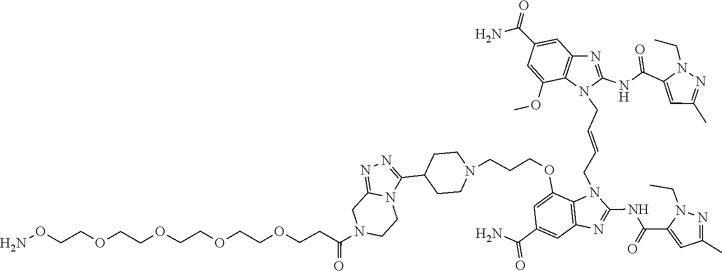 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCON)C5)CC3)c21] ¹H NMR (400 MHz, DMSO) δ 10.55 (s, 3H), 8.00 (d, J = 19.8 Hz, 2H), 7.68 (d, J = 5.3 Hz, 2H), 7.48-7.23 (m, 4H), 6.54 (d, J = 5.7 Hz, 3H), 5.91-5.65 (m, 5H), 5.04-4.75 (m, 9H), 4.54 (dd, J = 16.0, 7.5 Hz, 5H), 3.79-3.59 (m, 7H), 3.58-3.34 (m, 12H), 3.11 (s, 2H), 2.93 (d, J = 39.0 Hz, 3H), 2.82-2.60 (m, 3H), 2.12 (d, J = 11.3 Hz, 8H), 1.92 (d, J = 13.0 Hz, 4H), 1.28 (dt, J = 14.6, 7.1 Hz, 6H). LC-MS [ESI]: Calculated for C₅₉H₈₁N₁₈O₁₂ [M + H⁺]: 1233.62, Found: 1233.65. |
| LP-4-3 | 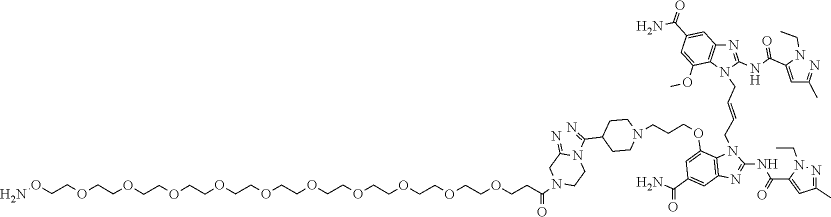 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCOCCOCCOCCOCCOCCOCCON)C5)CC3)c21] ¹H NMR (600 MHz, dmso) δ 7.98 (d, J = 29.2 Hz, 2H), 7.64 (d, J = 8.0 Hz, 2H), 7.38 (s, 2H), 7.29 (d, J = 25.0 Hz, 2H), 6.50 (d, J = 10.6 Hz, 2H), 5.73 (d, J = 35.7 Hz, 2H), 5.16-4.68 (m, 7H), 4.50 (dd, J = 25.4, 7.1 Hz, 3H), 4.16-3.80 (m, 7H), 3.78-3.55 (m, 6H), 3.49 (s, 5H), 3.20-2.55 (m, 3H), 2.22-1.71 (m, 11H), 1.24 (dt, J = 23.3, 7.1 Hz, 6H). LC-MS [ESI]: Calculated for C₇₁H₁₀₅N₁₈O₁₈ [M + H⁺]: 1497.78, Found: 1497.8. |
| LP-4-4 | 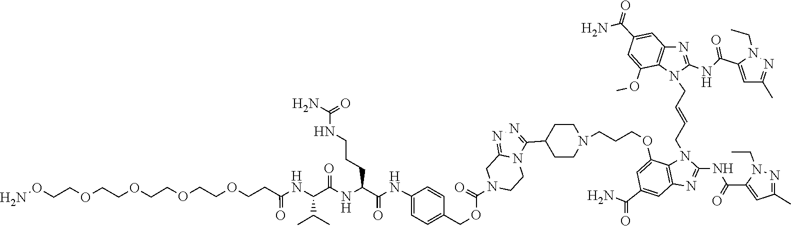 ¹H NMR (400 MHz, DMSO) δ 10.04 (s, 2H), 8.15 (d, J = 7.5 Hz, 1H), 7.99 (d, J = 17.4 Hz, 2H), 7.88 (d, J = 8.7 Hz, 1H), 7.71-7.65 (m, 2H), 7.65-7.59 (m, 2H), 7.45-7.28 (m, 5H), 6.54 (d, J = 7.8 Hz, 2H), 6.02 (s, 1H), 5.86-5.67 (m, 3H), 5.10 (s, 4H), 4.92 (d, J = 15.7 Hz, 6H), 4.76 (s, 5H), 4.54 (dd, J = 17.1, 7.2 Hz, 6H), 4.39 (q, J = 7.4 Hz, 2H), 4.24 (dd, J = 8.7, 6.7 Hz, 2H), 4.13-4.05 (m, 2H), 4.01 (s, 4H), 3.89 (s, 2H), 3.71 (s, 3H), 3.69-3.56 (m, 4H), 3.56- 3.37 (m, 12H), 3.10 (s, 2H), 2.99 (d, J = 20.3 Hz, 3H), 2.86 (s, 2H), 2.46-2.29 (m, 2H), 2.20-2.00 (m, 7H), 2.00-1.76 (m, 5H), 1.28 (dt, J = 17.0, 7.1 Hz, 6H), 0.85 (dd, J = 13.1, 6.8 Hz, 6H). LC-MS [ESI]: Calculated for C₇₈H₁₀₇N₂₃O₁₇ [M + H⁺]: 1638.82, Found: 1638.84. |
| LP-4-5 | 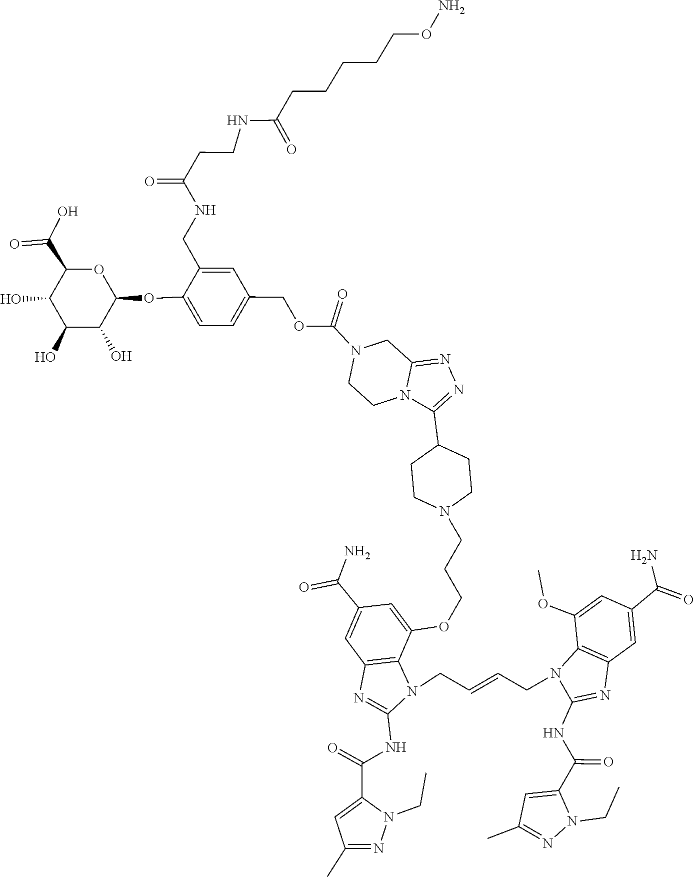 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(O[C@@H]6O[C@H](C(=O)O)[C@@H](O)[C@H](O)[C@H]6O)c(CNC(=O)CCNC(=O)CCCCCON)c4)C5)CC3)c21] ¹H NMR (400 MHz, DMSO) δ 8.21 (t, J = 6.1 Hz, 1H), 7.99 (d, J = 17.6 Hz, 2H), 7.83 (t, J = 5.6 Hz, 1H), 7.67 (d, J = 6.0 Hz, 2H), 7.39 (s, 2H), 7.32 (d, J = 11.2 Hz, 3H), 7.21 (s, 1H), 7.08 (d, J = 8.6 Hz, 1H), 6.53 (d, J = 7.2 Hz, 2H), 5.77 (q, J = 15.5 Hz, 3H), 5.08 (s, 2H), 4.94 (dd, J = 21.2, 10.0 Hz, 6H), 4.74 (s, 3H), 4.58-4.45 (m, 5H), 4.38-4.22 (m, 3H), 4.00 (s, 5H), 3.94-3.84 (m, 7H), 3.71 (s, 5H), 3.65 (s, 12H), 3.43 (d, J = 9.0 Hz, 4H), 3.34-3.31 (m, 2H), 3.24 (t, J = 6.4 Hz, 3H), 3.10 (s, 2H), 2.97-2.82 (m, 3H), 2.31 (t, J = 7.6 Hz, 3H), 2.15-2.00 (m, 12H), 1.91 (d, J = 9.9 Hz, 4H), 1.55 (t, J = 7.6 Hz, 2H), 1.48 (p, J = 7.0 Hz, 2H), 1.33-1.21 (m, 9H). LC-MS [ESI]: Calculated for C₇₂H₉₃N₂₀O₁₈ [M + H⁺]: 1525.69, Found: 1525.7. |
| LP-4-6 | 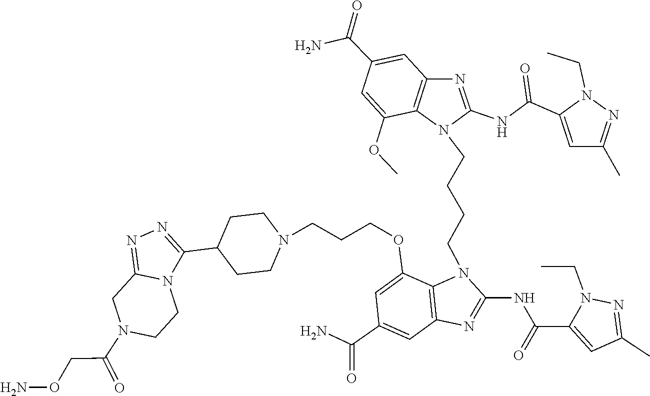 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCN(C(=O)CON)C5)CC3)c21] LC-MS [ESI]: Calculated for C₅₀H₆₅N₁₈O₈ [M + H⁺]: 1045.52, Found: 1045.6. 1H NMR (400 MHz, DMSO) 8 12.81 (s, 2H), 9.25 (s, 1H), 8.01 (s, 2H), 7.55 (s, 2H), 7.40 (s, 2H), 7.32 (s, 1H), 6.61 (s, 1H), 6.52 (s, 10H), 4.79 (s, 2H), 4.61-4.54 (m, 7H), 4.35 (s, 3H), 4.16 (s, 3H), 4.03 (s, 1H), 3.95 (s, 2H), 3.89 (s, 1H), 3.84 (d, J = 9.0 Hz, 4H), 3.14 (s, 1H), 2.92 (s, 3H), 2.61 (s, 2H), 2.54 (s, 2H), 2.11 (dd, J = 6.2, 2.8 Hz, 6H), 2.05(s, 6H), 1.86 (s, 5H), 1.32 (q, J = 7.1 Hz, 6H). |
| LP-4-7 | 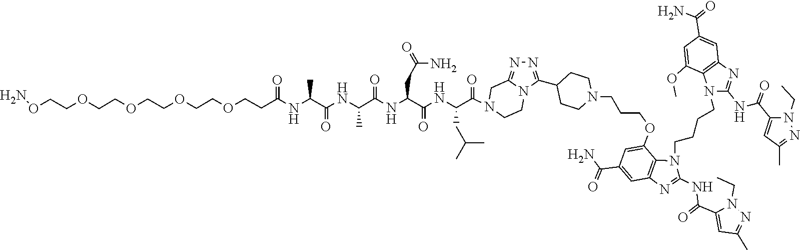 1H NMR (400 MHz, DMSO) δ 12.86 (s, 2H), 11.03 (s, 1H), 9.96 (s, 1H), 8.16- 8.05 (m, 2H), 7.96 (d, J = 18.0 Hz, 2H), 7.85 (d, J = 8.7 Hz, 1H), 7.67 (d, J = 3.8 Hz, 2H), 7.58 (d, J = 8.3 Hz, 2H), 7.38-7.24 (m, 6H), 6.95-6.89 (m, 1H), 6.53 (d, J = 6.3 Hz, 2H), 5.99 (s, 1H), 5.78 (q, J = 15.8 Hz, 2H), 5.41 (s, 2H), 4.95 (dd, J = 24.7, 12.7 Hz, 7H), 4.71 (d, J = 16.9 Hz, 1H), 4.58-4.48 (m, 3H), 4.38 (q, J = 7.3 Hz, 1H), 4.23 (dd, J = 8.7, 6.6 Hz, 1H), 4.08-4.01 (m, 2H), 3.71 (s, 3H), 3.66-3.56 (m, 3H), 2.94 (s,6H), 2.49-2.34 (m, 2H), 2.12 (d, J = 10.9 Hz, 5H), 2.01-1.90 (m, 2H), 1.28 (dt, J = 14.6, 7.2 Hz, 6H), 0.87 (ddd, J = 19.2, 14.5, 6.8 Hz, 12H). LC-MS [ESI]: Calculated for C₈₄H₁₁₉N₂₄O₁₈ [M + H⁺]: 1751.91, Found: 1751.92. |

### Synthesis 3H. LP-25-1 and LP-25-2

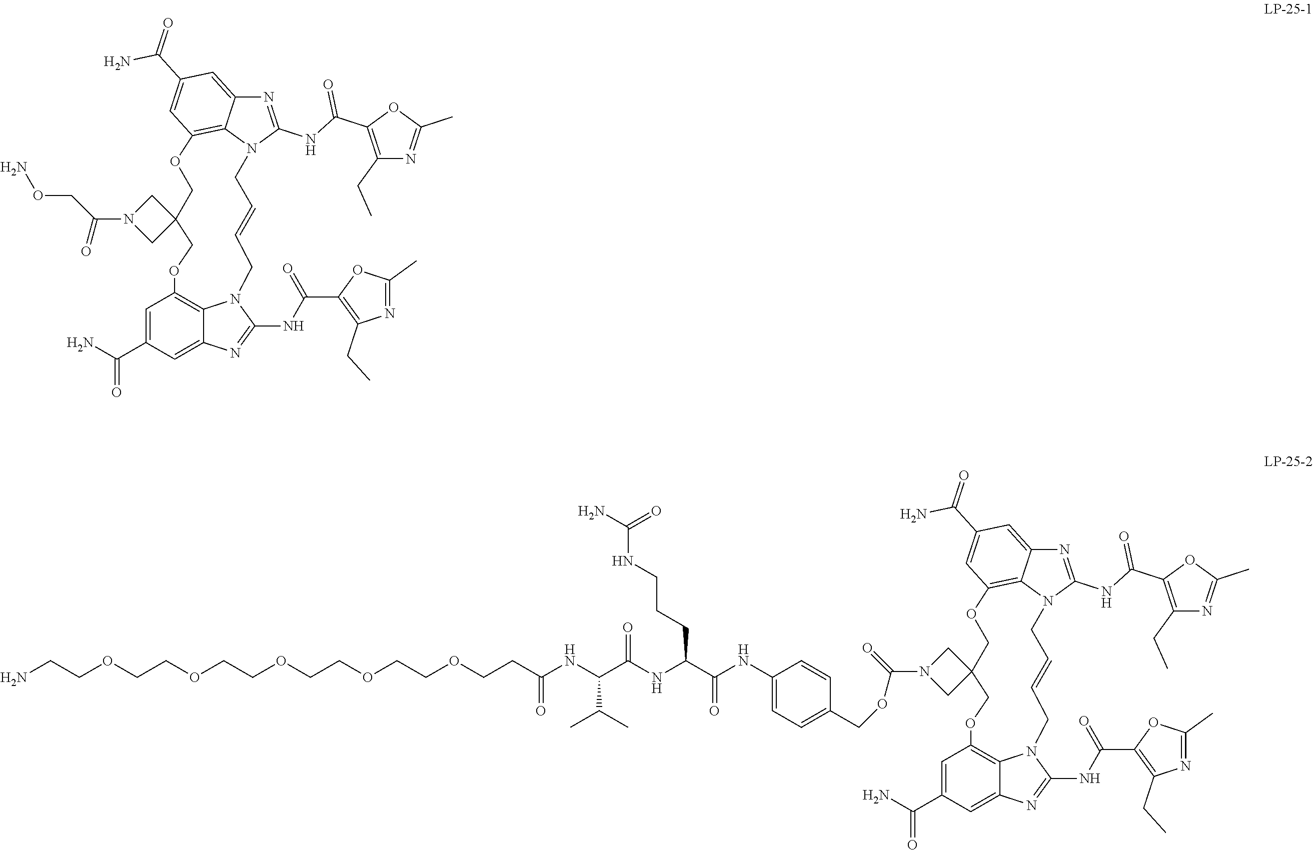 [SMILES: CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CO3)CN(C(=O)CON)C1 ; CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CO3)CN(C(=O)OCc2ccc(NC(=O)[C@H](CCCNC(N)=O)NC(=O)[C@@H](NC(=O)CCOCCOCCOCCOCCOCCN)C(C)C)cc2)C1]

LP-25-1 and LP-25-2 were synthesized as described herein from Compound 25.

LP-25-1: ¹H NMR (400 MHz, DMSO) δ 12.83 (s, 2H), 8.00 (s, 2H), 7.80 (d, J=1.4 Hz, 2H), 7.76 (d, J=1.3 Hz, 2H), 7.44 (s, 2H), 5.36 (s, 2H), 4.82 (s, 4H), 4.63-4.49 (m, 4H), 4.45 (s, 2H), 4.21 (s, 2H), 3.99 (s, 2H), 2.78 (q, J=7.5 Hz, 4H), 2.37 (s, 6H), 0.97 (t, J=7.5 Hz, 6H). LC-MS [ESI]: Calculated for C₄₁H₄₅N₁₂O₁₀ [M+H⁺]: 865.33, Found: 865.4.

LP-25-2: ¹H NMR (400 MHz, DMSO) δ 10.03 (s, 1H), 8.14 (d, J=7.4 Hz, 1H), 8.00 (s, 2H), 7.89 (d, J=8.7 Hz, 1H), 7.84 (s, 2H), 7.77 (d, J=1.2 Hz, 2H), 7.61 (d, J=8.5 Hz, 2H), 7.45 (s, 2H), 7.35 (d, J=8.4 Hz, 2H), 6.03 (s, 1H), 5.33 (s, 2H), 5.05 (s, 2H), 4.80 (s, 4H), 4.55 (s, 4H), 4.39 (q, J=7.5 Hz, 1H), 4.24 (dd, J=8.7, 6.6 Hz, 1H), 4.11-4.05 (m, 2H), 3.97 (d, J=16.7 Hz, 4H), 3.66-3.62 (m, 6H), 2.77 (q, J=7.5 Hz, 4H), 2.37 (s, 6H), 1.98 (dt, J=13.5, 6.8 Hz, 1H), 1.69 (t, J=7.7 Hz, 1H), 1.60 (dd, J=9.3, 4.9 Hz, 1H), 1.41 (dt, J=25.0, 5.5 Hz, 3H), 0.96 (t, J=7.5 Hz, 6H), 0.85 (dd, J=13.2, 6.8 Hz, 6H). LC-MS [ESI]: Calculated for C₆₉H₉₀N₁₇O₁₉ [M+H⁺]: 1460.65, Found: 1460.7.

### Synthesis 31. LP-5-1

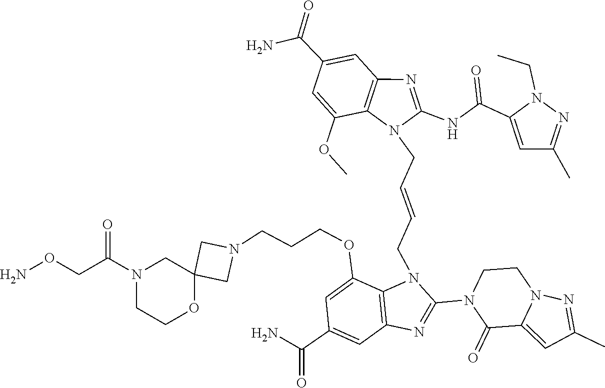 [SMILES: CCn1nc(C)cc1C(=O)Nc1nc2cc(C(N)=O)cc(OC)c2n1C/C=C/Cn1c(N2CCn3nc(C)cc3C2=O)nc2cc(C(N)=O)cc(OCCCN3CC4(C3)CN(C(=O)CON)CCO4)c21]

LP-5-1 is synthesized as described herein from Compound 5.

### Synthesis 3J. LP-21-1

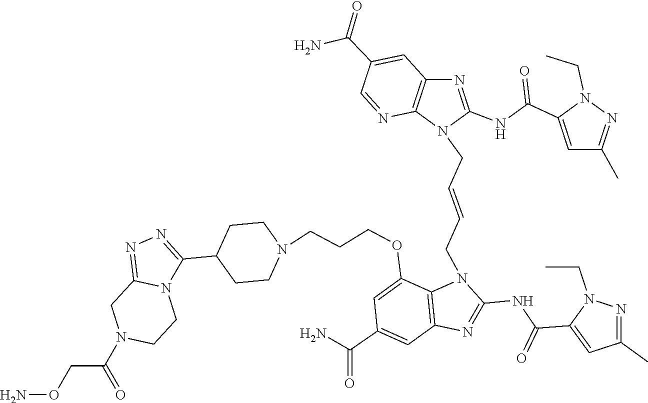 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CON)C5)CC3)c21]

LP-21-1 was synthesized using the same methods as described herein from Compound 21. ¹H NMR (400 MHz, DMSO) δ 12.89 (s, 3H), 10.04 (d, J=73.2 Hz, 3H), 8.74 (d, J=10.7 Hz, 2H), 8.33-7.90 (m, 4H), 7.62 (d, J=38.7 Hz, 3H), 7.37 (d, J=9.6 Hz, 3H), 6.58-6.44 (m, 2H), 6.06-5.90 (m, 2H), 5.75 (d, J=15.7 Hz, 2H), 5.04-4.74 (m, 9H), 4.51 (dq, J=14.0, 7.3 Hz, 6H), 4.23-3.90 (m, 13H), 3.86 (s, 3H), 3.54 (d, J=11.5 Hz, 2H), 3.33 (d, J=54.7 Hz, 4H), 3.00 (s, 3H), 2.61 (s, 1H), 2.25-1.82 (m, 12H), 1.26 (td, J=7.1, 5.0 Hz, 6H). LC-MS [ESI]: Calculated for C₄₈H₆₀N₁₉O₇ [M+H⁺]: 1014.48, Found: 1014.5.

### Synthesis 3K. LP-1-4

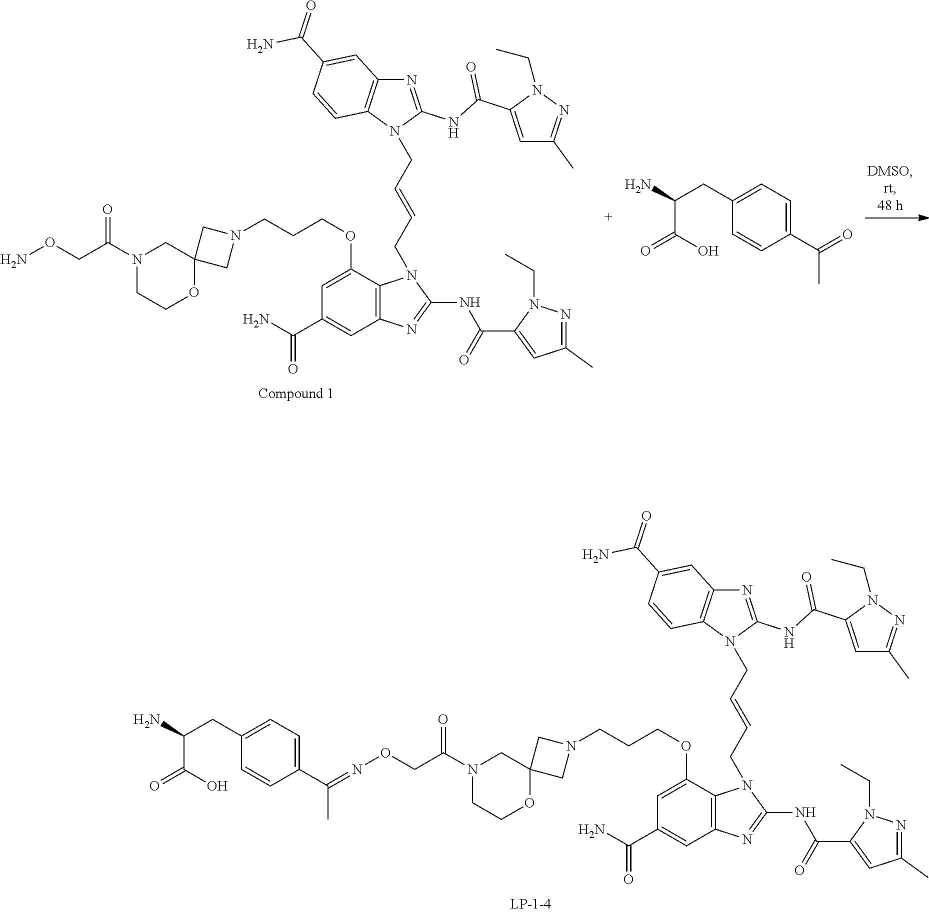 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)CON)CCO4)c21 ; CC(=O)c1ccc(C[C@H](N)C(=O)O)cc1 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)CON=C(C)c3ccc(C[C@H](N)C(=O)O)cc3)CCO4)c21]

To a stirred solution of 7-[3-[8-(2-aminooxyacetyl)-5-oxa-2,8-diazaspiro[3.5]nonan-2-yl]propoxy]-1-[(E)-4-[5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]benzimidazole-5-carboxamide (Compound LP-1-1, 4 mg) in DMSO (0.5 mL) at room temperature was added the solution of (2S)-3-(4-acetylphenyl)-2-amino-propanoic acid L-pAcF (1 mg). The reaction mixture was stirred at room temperature for 48 hours. LCMS showed complete conjugation was observed. Crude material was purified by prep-HPLC (Column: Phenomenex kinetex 5 μm C₁₈ 100 Å, 250×21.2 mm and using 10-90% ACN: 0.1% TFA in water as eluent, Flow rate: 21.2 mL/min, run time: 45 mins to afford rac-(2S)-2-amino-3-[4-[N-[2-[2-[3-[6-carbamoyl-2-[(2-ethyl-5-methyl-pyrazole-3-carbonyl)amino]-3-[rac-(E)-4-[5-carbamoyl-2-[(2-ethyl-5-methyl-pyrazole-3-carbonyl)amino]benzimidazol-1-yl]but-2-enyl]benzimidazol-4-yl]oxypropyl]-5-oxa-2,8-diazaspiro[3.5]nonan-8-yl]-2-oxo-ethoxy]-C-methyl-carbonimidoyl]phenyl]propanoic acid (LP-1-4, 3.5 mg) as a white solid. ¹H NMR (400 MHz, DMSO) δ 12.86 (d, J=23.7 Hz, 2H), 8.25 (s, 3H), 7.97 (d, J=8.0 Hz, 3H), 7.77-7.56 (m, 4H), 7.34 (dt, J=27.2, 7.9 Hz, 5H), 6.53 (d, J=12.1 Hz, 4H), 5.97 (d, J=15.4 Hz, 2H), 5.72 (s, 2H), 5.03-4.79 (m, 5H), 4.53 (d, J=7.0 Hz, 4H), 4.06 (t, J=63.2 Hz, 8H), 3.21-3.02 (m, 3H), 2.24 (s, 2H), 2.12 (s, 5H), 1.28 (td, J=7.1, 3.3 Hz, 6H). LC-MS [ESI]: Calculated for C₅₆H₆₆N₁₆O₁₀ [M+H⁺]: 1123.52, Found: 1123.40.

### Synthesis 3L. Expected pAcF Catabolites of Compound 4 Non-Cleavable Linker Payload-Based Protein Conjugates (LP-4-8, LP-4-9, and LP-4-10)

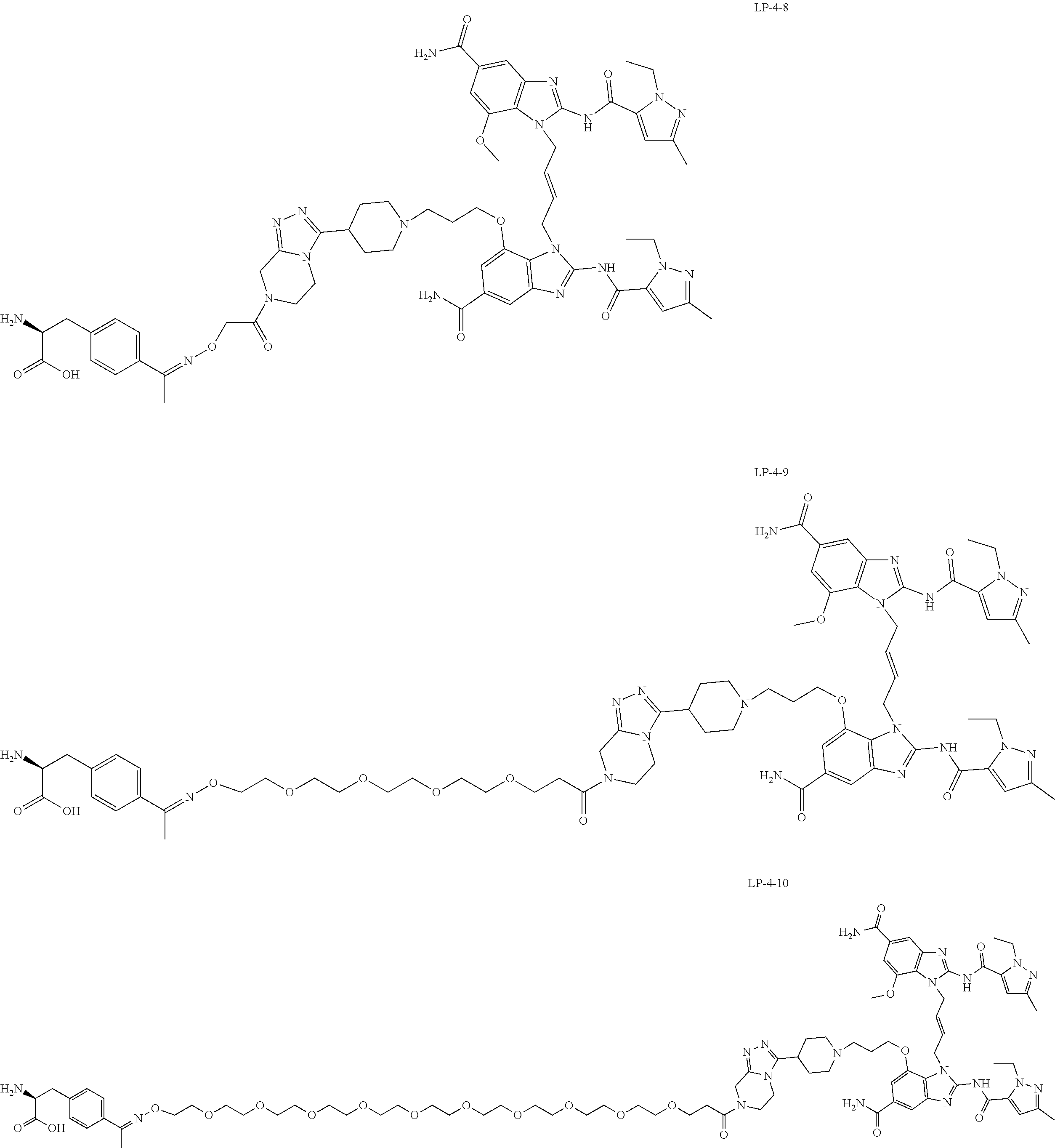

LP-4-8, LP-4-9, and LP-4-10 (expected pAcF catabolites of Compound 4 non-cleavable linker payload-based protein conjugates) are synthesized using the same methods as described herein from Compound 4.

### Synthesis 3M. Expected Protein Conjugate Catabolite pAcF Adduct of Compound 21-Based Linker Payload (LP-21-3)

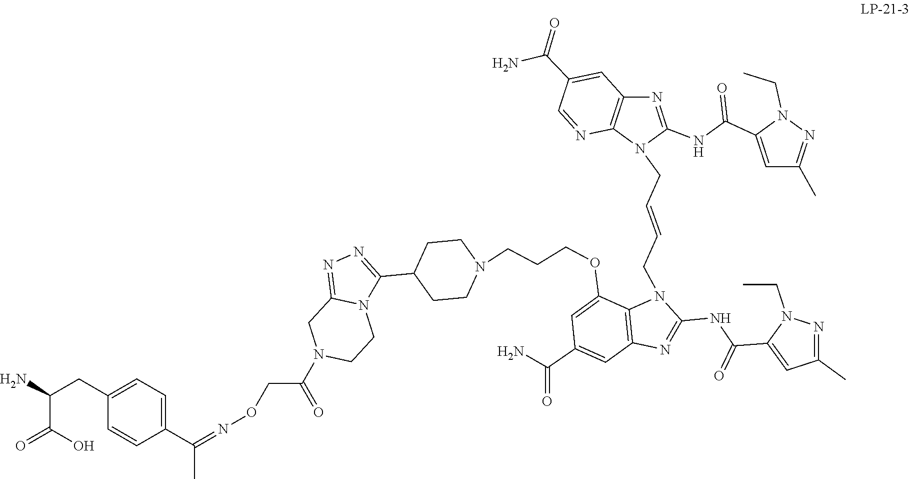 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(C[C@H](N)C(=O)O)cc4)C5)CC3)c21]

LP-21-3 is synthesized using the same methods as described herein from Compound 21.

### Synthesis 3N. LP-4-12 (Aminooxy-PEG4-vcpAB-Compound 4)

LP-4-12 was synthesized as described in Scheme 23.

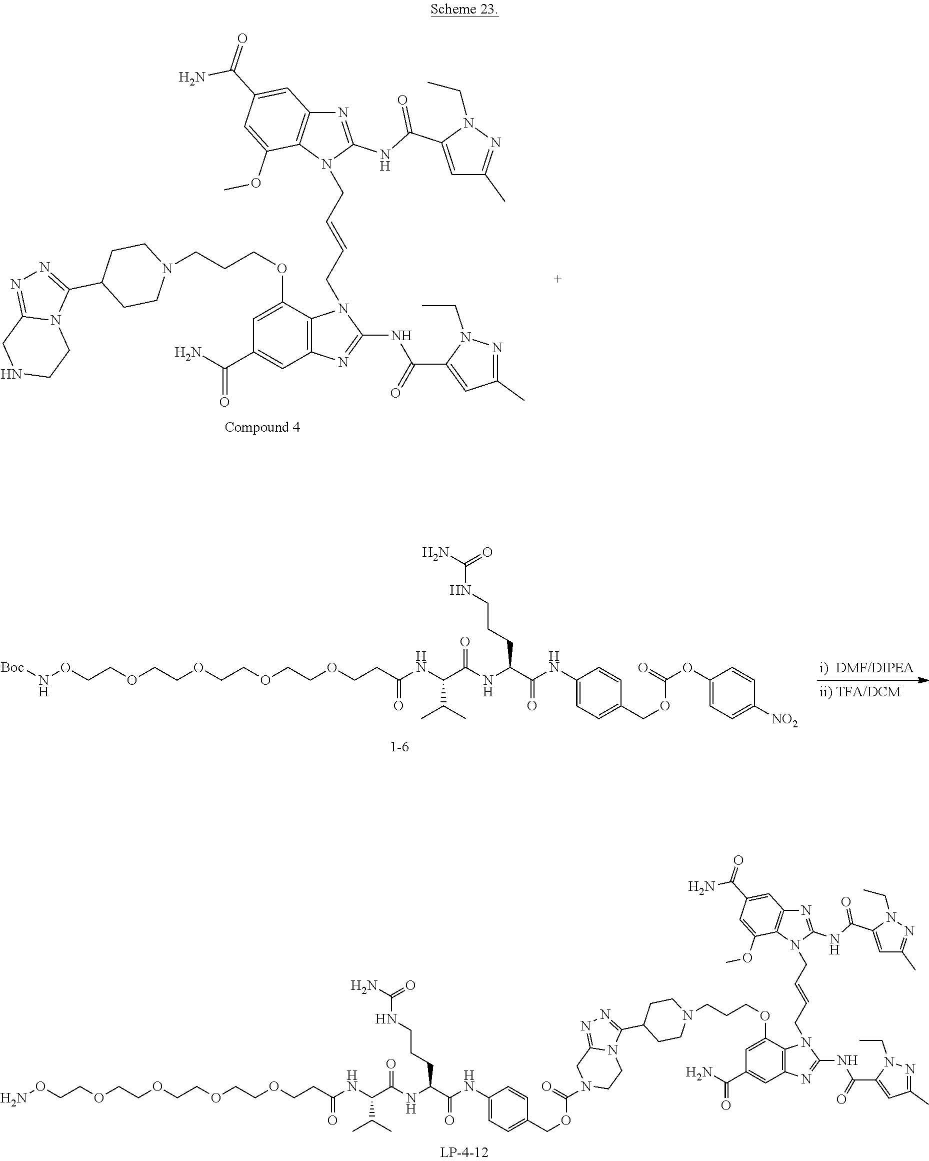 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCNC5)CC3)c21 ; CC(C)[C@H](NC(=O)CCOCCOCCOCCOCCONC(=O)OC(C)(C)C)C(=O)N[C@@H](CCCNC(N)=O)C(=O)Nc1ccc(COC(=O)Oc2ccc([N+](=O)[O-])cc2)cc1 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(NC(=O)[C@H](CCCNC(N)=O)NC(=O)[C@@H](NC(=O)CCOCCOCCOCCOCCON)C(C)C)cc4)C5)CC3)c21]

To a solution of compound Compound 4 (45 mg) in anhydrous DMF (2 mL) at RT was added compound Boc-NO-PEG4-VC-PAB-PNP (1-6, 28.8 mg) followed by DIPEA (45 uL). The mixture was stirred at room temperature over 24 hours. The crude mixture was directly purified by reverse phase preparative HPLC to give Boc protected LP-4-12 as a white powder after lyophilization (56 mg). LC-MS [ESI]: Calculated for C₈₃H₁₁₆N₂₃O₁₉ [M+H⁺]: 1738.87, Found: 1738.9.

Boc protected LP-4-12 (56 mg) was treated with TFA/DCM (1/4, v/v, 2 mL) at room temperature for 10 minutes. After which, LC-MS showed the desired product formation, The reaction mixture was evaporated to dryness under reduced pressure and the residue was purified by reverse phase preparative HPLC to give LP-4-12 as a white powder (46 mg). LC-MS [ESI]: Calculated for C₇₈H₁₀₇N₂₃O₁₇ [M+H⁺]: 1638.82, Found: 1638.9. ¹H NMR (400 MHz, DMSO) δ 10.04 (s, 2H), 8.15 (d, J=7.5 Hz, 1H), 7.99 (d, J=17.4 Hz, 2H), 7.88 (d, J=8.7 Hz, 1H), 7.71-7.65 (m, 2H), 7.65-7.59 (m, 2H), 7.45-7.28 (m, 5H), 6.54 (d, J=7.8 Hz, 2H), 6.02 (s, 1H), 5.86-5.67 (m, 3H), 5.10 (s, 4H), 4.92 (d, J=15.7 Hz, 6H), 4.76 (s, 5H), 4.54 (dd, J=17.1, 7.2 Hz, 6H), 4.39 (q, J=7.4 Hz, 2H), 4.24 (dd, J=8.7, 6.7 Hz, 2H), 4.13-4.05 (m, 2H), 4.01 (s, 4H), 3.89 (s, 2H), 3.71 (s, 3H), 3.69-3.56 (m, 4H), 3.56-3.37 (m, 12H), 3.10 (s, 2H), 2.99 (d, J=20.3 Hz, 3H), 2.86 (s, 2H), 2.46-2.29 (m, 2H), 2.20-2.00 (m, 7H), 2.00-1.76 (m, 5H), 1.28 (dt, J=17.0, 7.1 Hz, 6H), 0.85 (dd, J=13.1, 6.8 Hz, 6H).

### Synthesis 3O. LP-4-11 (Aminooxy-β-Glucuronidase Cleavable-Compound 4)

LP-4-11 was synthesized as described in Scheme 24.

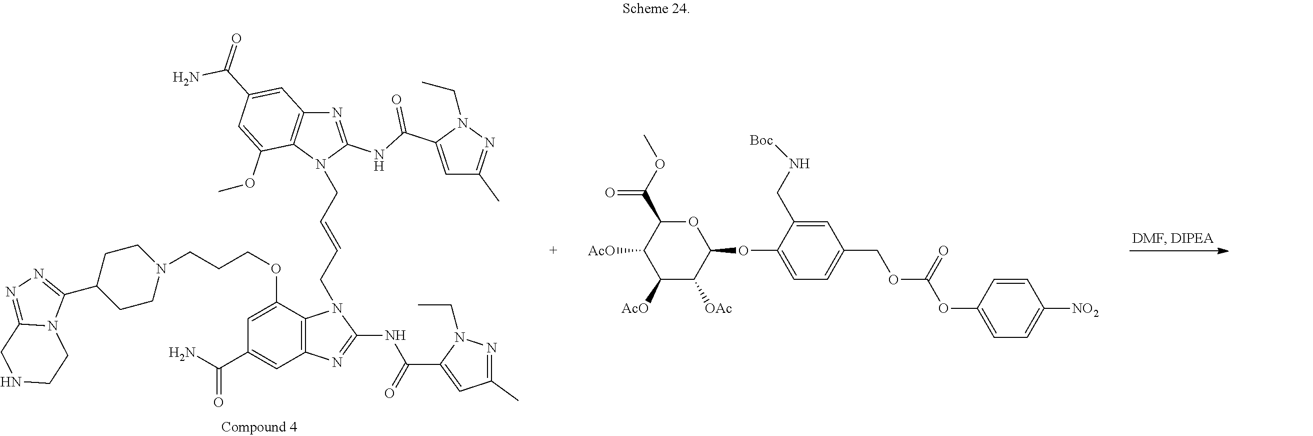 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCNC5)CC3)c21 ; COC(=O)[C@H]1O[C@@H](Oc2ccc(COC(=O)Oc3ccc([N+](=O)[O-])cc3)cc2CNC(=O)OC(C)(C)C)[C@H](OC(C)=O)[C@@H](OC(C)=O)[C@@H]1OC(C)=O]

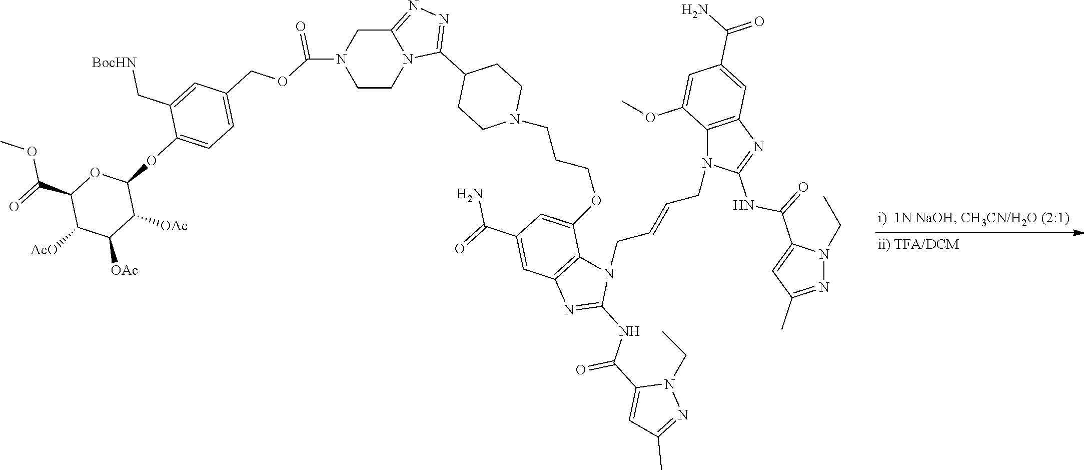 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(O[C@@H]6O[C@H](C(=O)OC)[C@@H](OC(C)=O)[C@H](OC(C)=O)[C@H]6OC(C)=O)c(CNC(=O)OC(C)(C)C)c4)C5)CC3)c21]

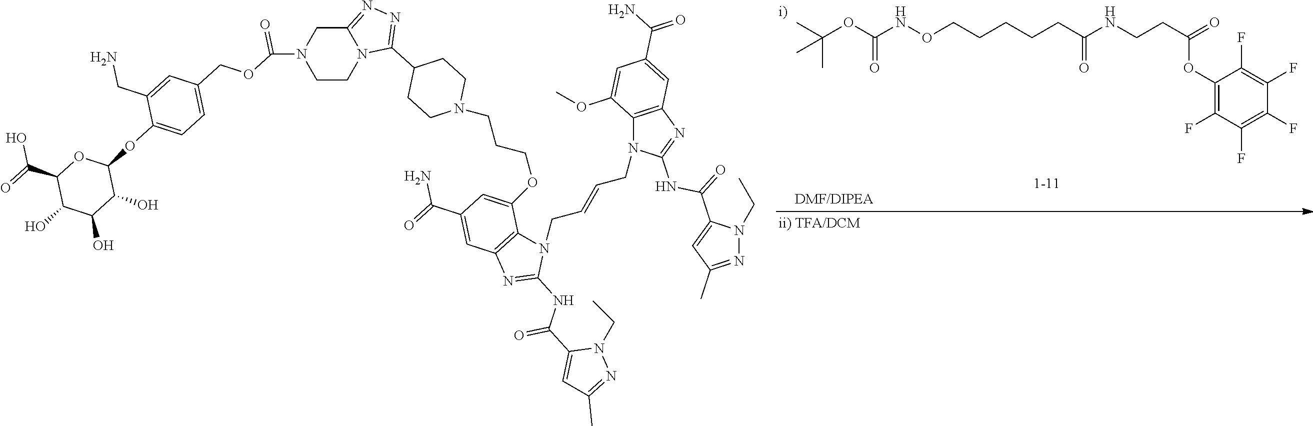 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(O[C@@H]6O[C@H](C(=O)O)[C@@H](O)[C@H](O)[C@H]6O)c(CN)c4)C5)CC3)c21 ; CC(C)(C)OC(=O)NOCCCCCC(=O)NCCC(=O)Oc1c(F)c(F)c(F)c(F)c1F]

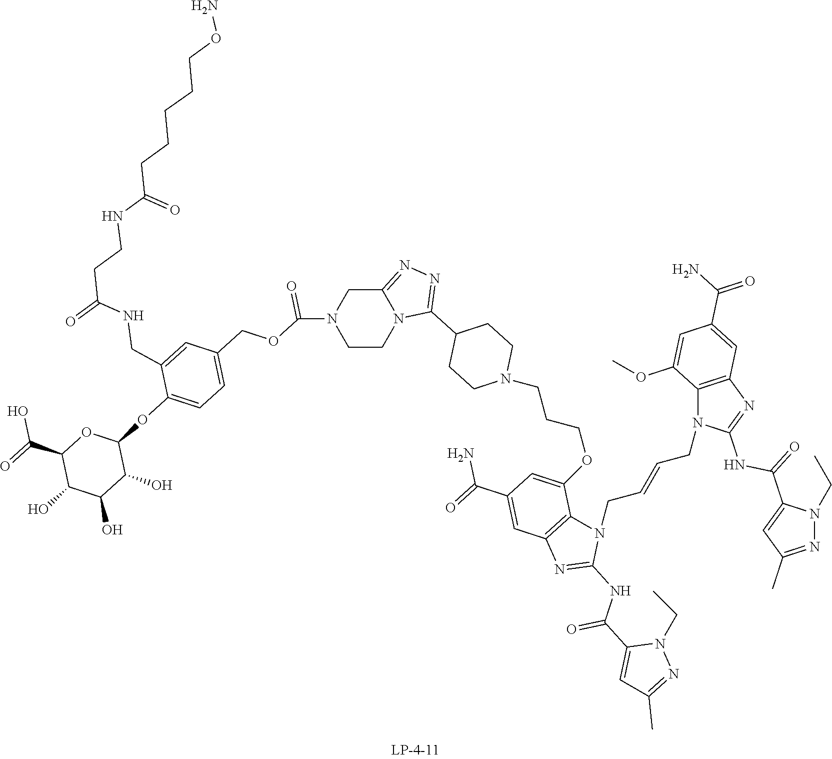 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(O[C@@H]6O[C@H](C(=O)O)[C@@H](O)[C@H](O)[C@H]6O)c(CNC(=O)CCNC(=O)CCCCCON)c4)C5)CC3)c21]

LP-4-11 was synthesized using the same methods described herein in an analogous fashion. LC-MS [ESI]: Calculated for C₇₂H₉₃N₂₀O₁₈ [M+H⁺]: 1525.69, Found: 1529.7. ¹H NMR (400 MHz, DMSO) δ 8.21 (t, J=6.1 Hz, 1H), 7.99 (d, J=17.6 Hz, 2H), 7.83 (t, J=5.6 Hz, 1H), 7.67 (d, J=6.0 Hz, 2H), 7.39 (s, 2H), 7.32 (d, J=11.2 Hz, 3H), 7.21 (s, 1H), 7.08 (d, J=8.6 Hz, 1H), 6.53 (d, J=7.2 Hz, 2H), 5.77 (q, J=15.5 Hz, 3H), 5.08 (s, 2H), 4.94 (dd, J=21.2, 10.0 Hz, 6H), 4.74 (s, 3H), 4.58-4.45 (m, 5H), 4.38-4.22 (m, 3H), 4.00 (s, 5H), 3.94-3.84 (m, 7H), 3.71 (s, 5H), 3.65 (s, 12H), 3.43 (d, J=9.0 Hz, 4H), 3.34-3.31 (m, 2H), 3.24 (t, J=6.4 Hz, 3H), 3.10 (s, 2H), 2.97-2.82 (m, 3H), 2.31 (t, J=7.6 Hz, 3H), 2.15-2.00 (m, 12H), 1.91 (d, J=9.9 Hz, 4H), 1.55 (t, J=7.6 Hz, 2H), 1.48 (p, J=7.0 Hz, 2H), 1.33-1.21 (m, 9H).

### Synthesis 3P. LP-4-14, LP-4-15, and LP-4-16

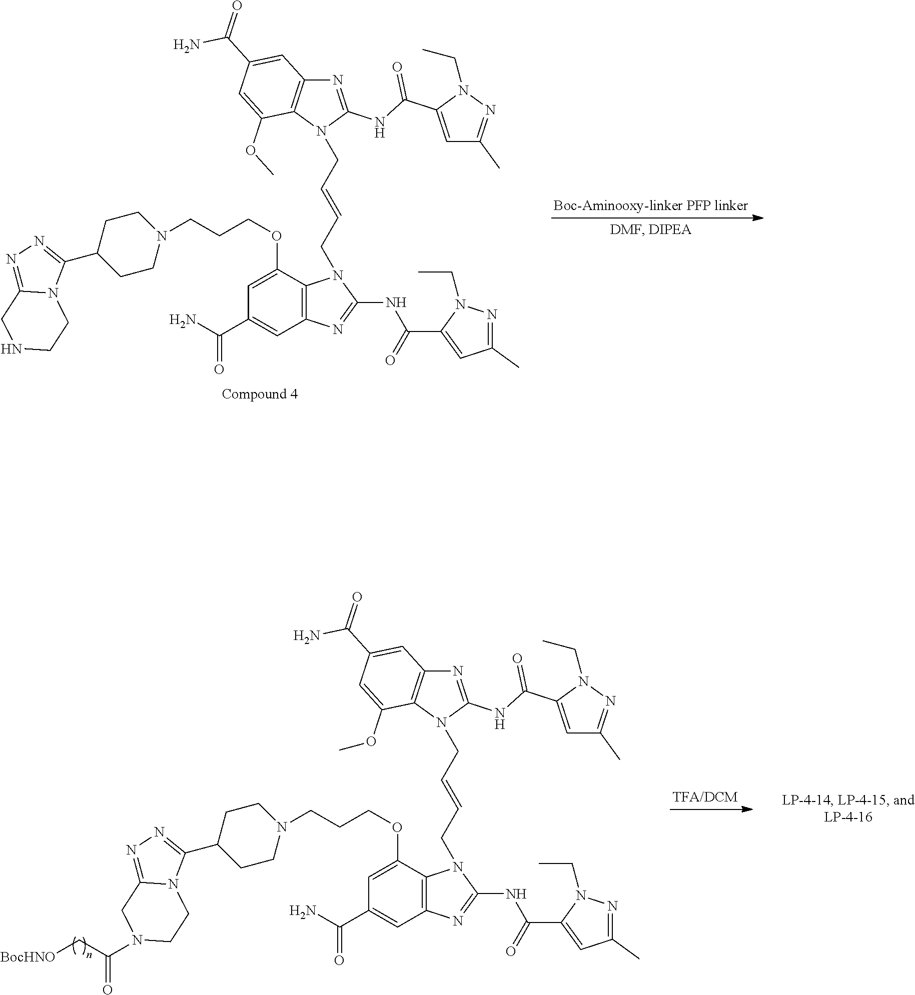

LP-4-14, LP-4-15, and LP-4-16 were synthesized using the general scheme as shown above.

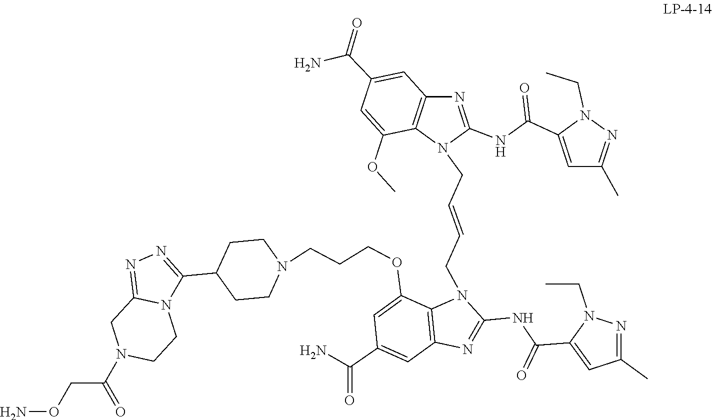 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21]

LP-4-14: LC-MS [ESI]: Calculated for C₅₀H₆₃N₁₈O₈ [M+H⁺]:1043.5, Found: 1043.6. ¹H NMR (400 MHz, DMSO) δ 9.35 (s, 2H), 8.01 (d, J=25.2 Hz, 3H), 7.68 (d, J=6.6 Hz, 3H), 7.55-7.13 (m, 5H), 6.72-6.26 (m, 3H), 6.00-5.62 (m, 3H), 4.92 (dd, J=12.7, 4.8 Hz, 5H), 4.78 (d, J=28.9 Hz, 5H), 4.53 (dt, J=14.1, 7.2 Hz, 6H), 4.10-3.93 (m, 8H), 3.88 (s, 3H), 3.73 (d, J=8.8 Hz, 5H), 3.53-3.37 (m, 3H), 2.93 (d, J=38.2 Hz, 3H), 2.18-2.02 (m, 8H), 1.93 (d, J=12.6 Hz, 4H), 1.29 (dt, J=14.4, 7.1 Hz, 6H).

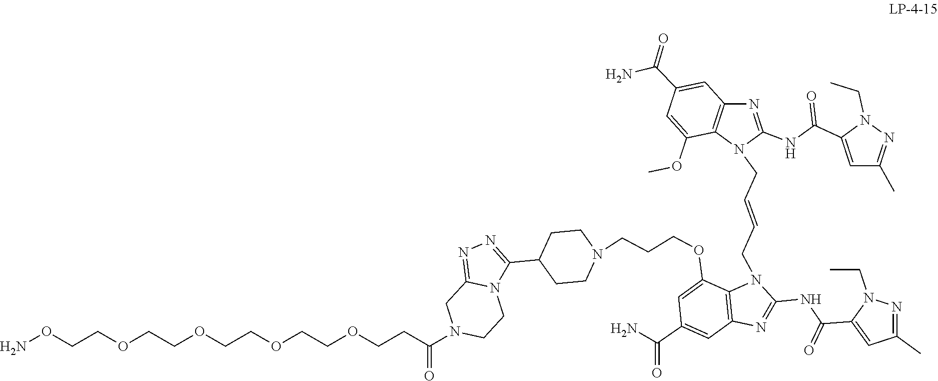 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCON)C5)CC3)c21]

LP-4-15: LC-MS [ESI]: Calculated for C₅₉H₈₁N₁₈O₁₂ [M+H⁺]:1233.62, Found: 1233.64. ¹H NMR (400 MHz, DMSO) δ 10.55 (s, 1H), 8.00 (d, J=19.8 Hz, 1H), 7.68 (d, J=5.3 Hz, 1H), 7.48-7.23 (m, 2H), 6.54 (d, J=5.7 Hz, 1H), 5.91-5.65 (m, 3H), 5.04-4.75 (m, 4H), 4.54 (dd, J=16.0, 7.5 Hz, 3H), 3.79-3.59 (m, 3H), 3.58-3.34 (m, 6H), 3.11 (s, 1H), 2.93 (d, J=39.0 Hz, 2H), 2.82-2.60 (m, 1H), 2.12 (d, J=11.3 Hz, 4H), 1.92 (d, J=13.0 Hz, 2H), 1.28 (dt, J=14.6, 7.1 Hz, 3H).

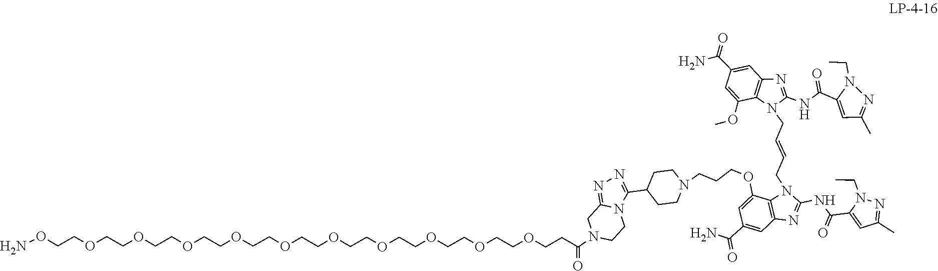 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCOCCOCCOCCOCCOCCOCCON)C5)CC3)c21]

LP-4-16: LC-MS [ESI]: Calculated for C₁₇H₁₀₅N₁₈O₁₈ [M+H⁺]:1497.78, Found: 1497.82. ¹HNMR (600 MHz, DMSO) δ 7.98 (d, J=29.2 Hz, 2H), 7.64 (d, J=8.0 Hz, 2H), 7.38 (s, 2H), 7.29 (d, J=25.0 Hz, 2H), 6.50 (d, J=10.6 Hz, 2H), 5.73 (d, J=35.7 Hz, 2H), 5.16-4.68 (m, 9H), 4.50 (dd, J=25.4, 7.1 Hz, 4H), 4.16-3.80 (m, 9H), 3.78-3.55 (m, 8H), 3.49 (s, 6H), 3.20-2.55 (m, 4H), 2.22-1.71 (m, 14H), 1.24 (dt, J=23.3, 7.1 Hz, 7H).

### Synthesis 3Q. Non-Cleavable Compound 49-Aminooxy LP-49-1

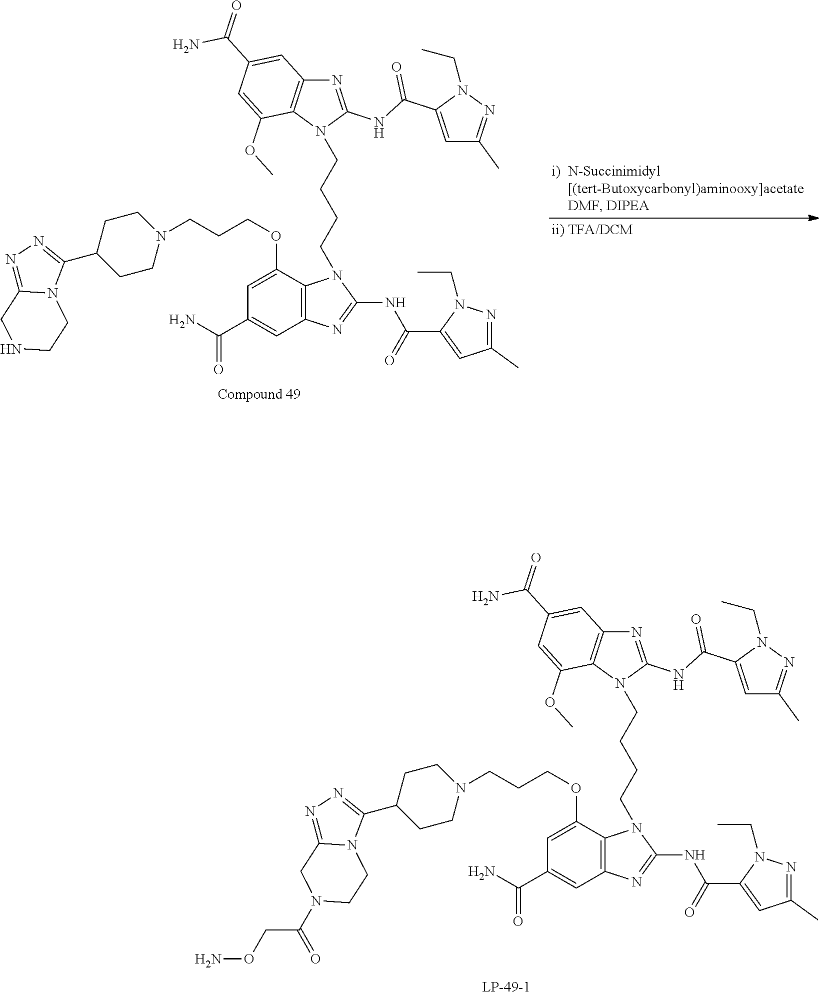 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCNC5)CC3)c21 ; CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCN(C(=O)CON)C5)CC3)c21]

LP-49-1 was synthesized using the same methods in an analogous fashion as described herein. LC-MS [ESI]: Calculated for C₅₀H₆₅N₁₈O₈ [M+H⁺]: 1045.52, Found: 1045.6. ¹HNMR (400 MHz, DMSO) δ 12.81 (s, 2H), 9.25 (s, 1H), 8.01 (s, 2H), 7.55 (s, 2H), 7.40 (s, 2H), 7.32 (s, 1H), 6.61 (s, 1H), 6.52 (s, 10H), 4.79 (s, 2H), 4.61-4.54 (i, 7H), 4.35 (s, 3H), 4.16 (s, 3H), 4.03 (s, 1H), 3.95 (s, 2H), 3.89 (s, 1H), 3.84 (d, J=9.0 Hz, 4H), 3.14 (s, 1H), 2.92 (s, 3H), 2.61 (s, 2H), 2.54 (s, 2H), 2.11 (dd, J=6.2, 2.8 Hz, 6H), 2.05 (s, 6H), 1.86 (s, 5H), 1.32 (q, J=7.1 Hz, 6H).

### Synthesis of LP-12-1

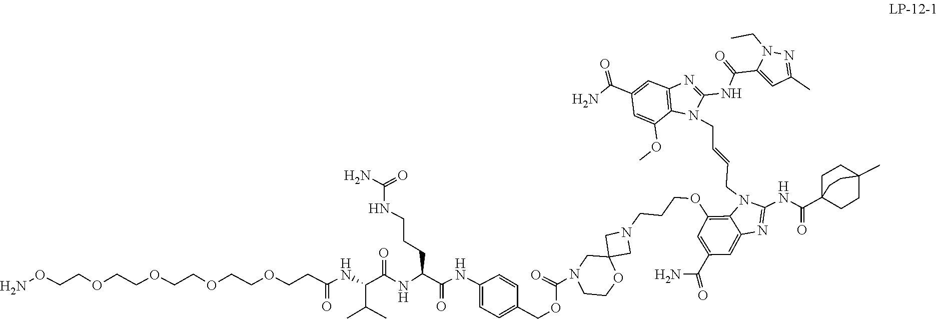 [SMILES: CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(C3)CN(C(=O)OCc3ccc(NC(=O)[C@H](CCCNC(N)=O)NC(=O)[C@@H](NC(=O)CCOCCOCCOCCOCCON)C(C)C)cc3)CCO4)c21]

LP-12-1 was synthesized and purified by reverse phase HPLC using the same methods as described above. LC-MS [ESI]: Calculated for C₇₇H₁₀₉N₁₈O₁₈ [M+H⁺]: 1573.81, observed 1573.83. ¹HNMR (400 MHz, DMSO) δ 12.83 (s, 1H), 10.45 (s, 4H), 10.02 (s, 1H), 8.14 (d, J=7.6 Hz, 1H), 7.98 (s, 2H), 7.88 (d, J=8.6 Hz, 1H), 7.60 (d, J=8.3 Hz, 2H), 7.35-7.28 (m, 5H), 6.53 (s, 1H), 6.02 (s, 1H), 5.76 (d, J=15.4 Hz, 1H), 5.59 (s, 1H), 5.04 (s, 2H), 4.82-4.77 (m, 2H), 4.51 (q, J=7.2 Hz, 2H), 4.36 (t, J=7.1 Hz, 1H), 4.23 (dd, J=8.7, 6.7 Hz, 1H), 4.07 (t, J=4.3 Hz, 4H), 3.82 (s, 63H), 3.72 (s, 2H), 3.71 (s, 19H), 3.61 (dq, J=16.6, 3.8 Hz, 5H), 3.59-3.43 (m, 12H), 3.32 (d, J=7.8 Hz, 4H), 2.98 (d, J=19.0 Hz, 1H), 2.46 (d, J=7.5 Hz, 1H), 2.43-2.35 (m, 1H), 2.13 (s, 3H), 1.87 (s, 3H), 1.62-1.54 (m, 6H), 1.23 (q, J=7.1 Hz, 9H), 0.84 (dd, J=12.5, 6.8 Hz, 6H), 0.73 (s, 3H).

### Synthesis 3R. Synthesis of LP-12-2

LP-12-52 was synthesized as described in Scheme 24.

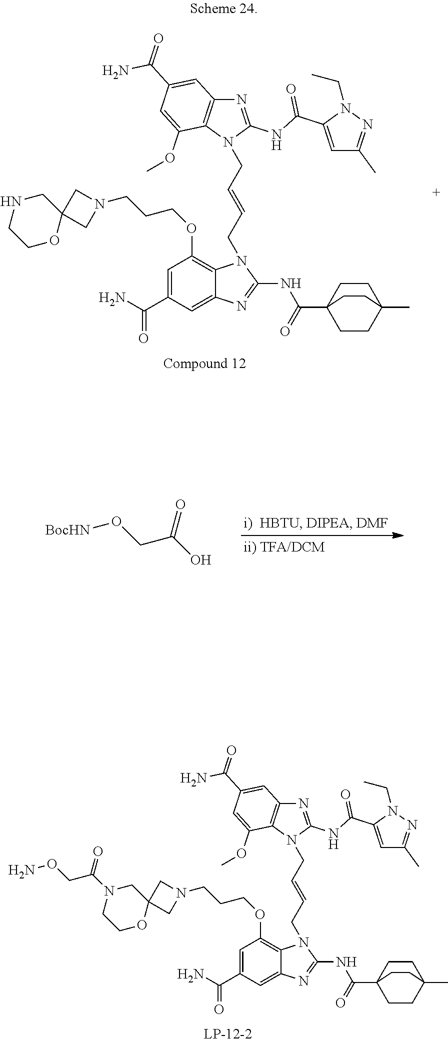 [SMILES: CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(CNCCO4)C3)c21 ; CC(C)(C)OC(=O)NOCC(=O)O ; CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(C3)CN(C(=O)CON)CCO4)c21]

A mixture of tert-butyl (E)-(2-(2-(3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(4-methylbicyclo[2.2.2]octane-1-carboxamido)-1H-benzo[d]imidazol-7-yl)oxy)propyl)-5-oxa-2,8-diazaspiro[3.5]nonan-8-yl)-2-oxoethoxy)carbamate (Compound 12, 14.4 mg, 0.013 mmol) in 2 mL of 5% (v/v) trifluoracetic acid in 1,1,1,3,3,3-hexafluoroisopropanol (HFIP) was stirred at room temperature for 1 hour. The reaction mixture was concentrated. The residue was diluted with DMSO (2 mL) and purified by reverse phase preparative HPLC to give LP-12-2 (7.8 mg). LC-MS [ESI]: Calculated for C₄₉H₆₄N₁₃O₉[M+H⁺]: 978.49, observed 978.5. ¹H NMR (400 MHz, DMSO) δ 7.98 (s, 1H), 7.73-7.64 (m, 2H), 7.40-7.29 (m, 4H), 6.54 (s, 1H), 5.60 (s, 1H), 4.86 (d, J=5.7 Hz, 2H), 4.78 (s, 2H), 4.61 (s, 2H), 4.51 (q, J=7.1 Hz, 2H), 4.09 (s, 4H), 3.94 (s, 2H), 3.73 (d, J=5.1 Hz, 5H), 3.33 (s, 4H), 2.14 (s, 3H), 1.89 (s, 2H), 1.59 (t, J=8.0 Hz, 6H), 1.24 (q, J=7.1 Hz, 9H), 0.74 (s, 3H).

### Synthesis 3S. Synthesis of LP-65-1

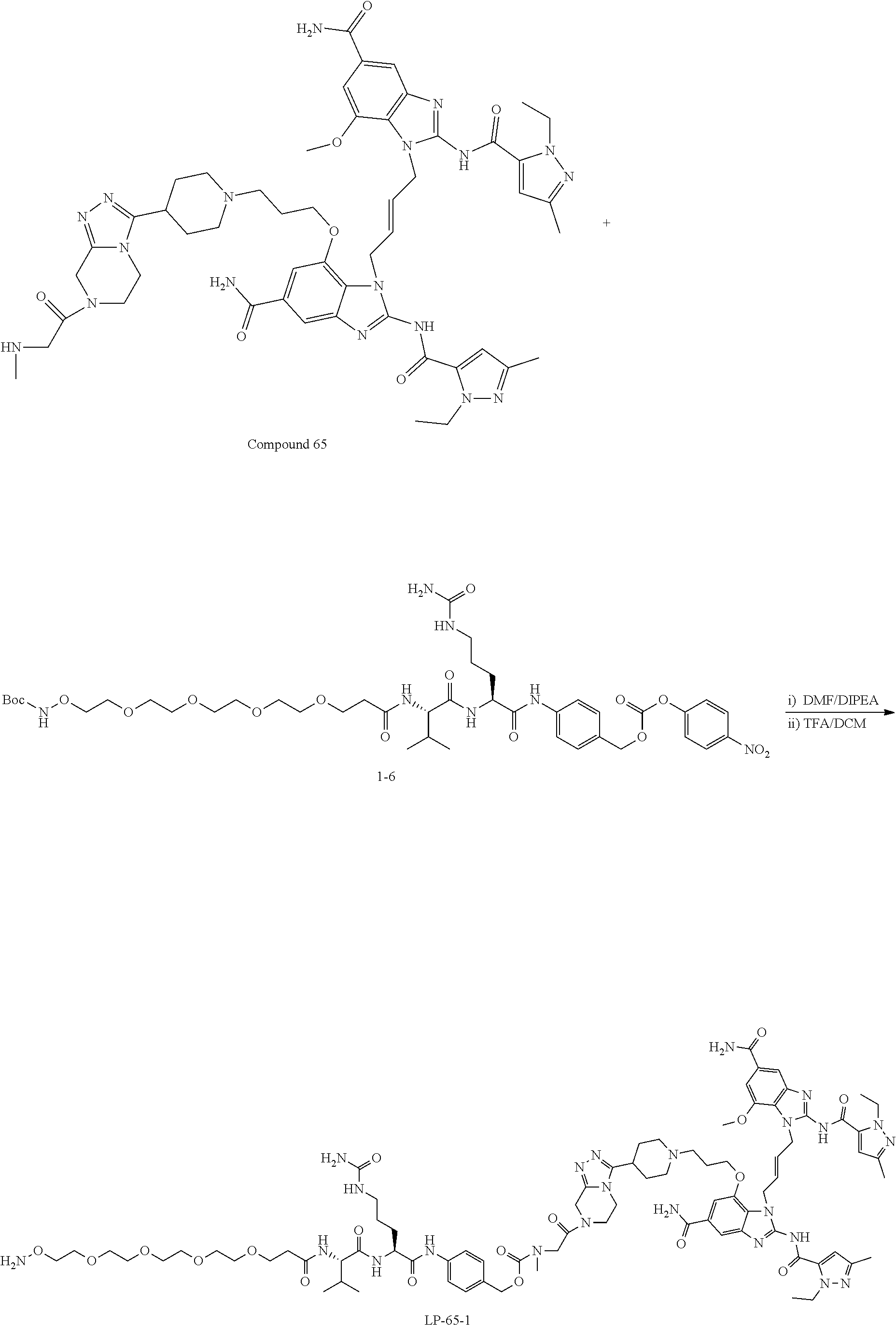 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CNC)C5)CC3)c21 ; CC(C)[C@H](NC(=O)CCOCCOCCOCCOCCONC(=O)OC(C)(C)C)C(=O)N[C@@H](CCCNC(N)=O)C(=O)Nc1ccc(COC(=O)Oc2ccc([N+](=O)[O-])cc2)cc1 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CN(C)C(=O)OCc4ccc(NC(=O)[C@H](CCCNC(N)=O)NC(=O)[C@@H](NC(=O)CCOCCOCCOCCOCCON)C(C)C)cc4)C5)CC3)c21]

Boc protected LP-65-1 was synthesized using the same methods as described above.

A mixture of floe protected LP-65-1 (30 mg, 0.0166 mmol) in 2 mL of 20% TFA in DCM was stirred at room temperature for 0.5 hour. The reaction mixture was concentrated. The residue was diluted with DMSO (2 mL) and purified by reverse phase preparative HPLC (acetonitrile/water+0.1% TFA) to afford 4-((17S,20S)-1-(aminooxy)-17-isopropyl-15,18-dioxo-20-(3-ureidopropyl)-3,6,9,12-tetraoxa-16,19-diazahenicosan-21-amido)benzyl (2-(3-(1-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-2-oxoethyl)(methyl)carbamate (LP-65-1) as a white solid, 21.8 mg). LCMS (ESI) m/z 1710.4 (M+H). ¹H NMR (400 MHz, DMSO) δ 12.89 (s, 2H), 11.04 (s, 1H), 10.01 (s, 1H), 8.16-8.08 (m, 2H), 8.01 (s, 1H), 7.96 (s, 1H), 7.87 (d, J=8.7 Hz, 1H), 7.67 (s, 1H), 7.60 (d, J=8.2 Hz, 1H), 7.55 (s, 1H), 7.38 (s, 3H), 7.31 (d, J=9.1 Hz, 2H), 7.24 (s, 1H), 6.97-6.89 (m, 2H), 6.53 (s, 3H), 5.99 (s, 1H), 5.77 (d, J=17.0 Hz, 2H), 5.42 (s, 2H), 5.00 (d, J=12.5 Hz, 2H), 4.92 (d, J=13.6 Hz, 3H), 4.86 (s, 1H), 4.79 (s, 1H), 4.53 (s, 4H), 4.38 (s, 1H), 4.32 (s, 1H), 4.23 (d, J=8.3 Hz, 2H), 4.00 (s, 7H), 3.94 (s, 2H), 3.72 (s, 3H), 3.65-3.58 (m, 4H), 3.50 (dd, J=8.5, 6.5 Hz, 11H), 3.10 (s, 2H), 3.01 (s, 1H), 2.95 (s, 3H), 2.86 (dd, J=18.6, 9.2 Hz, 4H), 2.12 (d, J=11.8 Hz, 5H), 2.05 (s, 2H), 1.94 (s, 4H), 1.90 (s, 1H), 1.69 (s, 1H), 1.59 (s, 1H), 1.27 (dd, J=16.3, 8.9 Hz, 7H), 0.89-0.79 (m, 6H).

### Synthesis 3T. LP-65-2

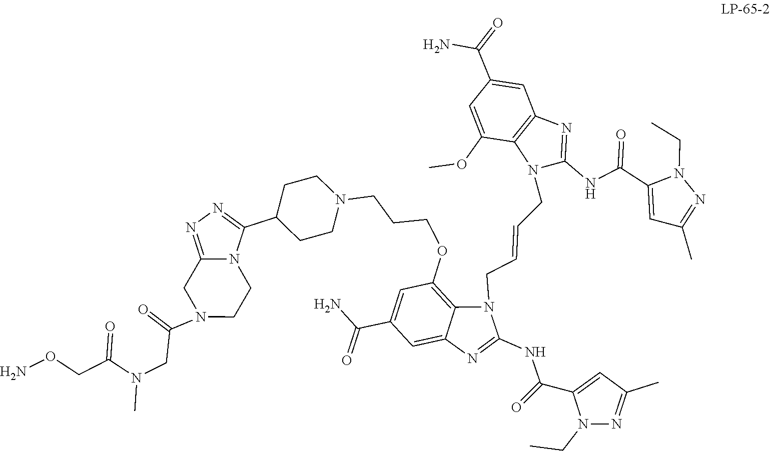 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CN(C)C(=O)CON)C5)CC3)c21]

LP-65-2 was synthesized in an analogous fashion using the same methods as described above. LCMS (ESI) m/z 1114.6 (M+H). ¹H NMR (400 MHz, DMSO) δ 12.88 (s, 2H), 9.36 (s, 1H), 8.01 (s, 1H), 7.96 (s, 1H), 7.68 (s, 1H), 7.41-7.28 (m, 4H), 6.57-6.47 (m, 2H), 5.80 (s, 1H), 5.75 (s, 1H), 4.91 (d, J=14.7 Hz, 4H), 4.86-4.77 (m, 2H), 4.74 (s, 1H), 4.59-4.48 (m, 5H), 4.44 (d, J=12.3 Hz, 1H), 4.37 (s, 1H), 4.01 (s, 5H), 3.96 (s, 1H), 3.72 (d, J=8.8 Hz, 3H), 3.10 (s, 1H), 2.89 (dd, J=24.3, 10.4 Hz, 4H), 2.60 (s, 1H), 2.11 (dd, J=10.0, 5.3 Hz, 7H), 1.91 (s, 4H), 1.34-1.21 (m, 6H).

### Synthesis 3U. LP-60-1

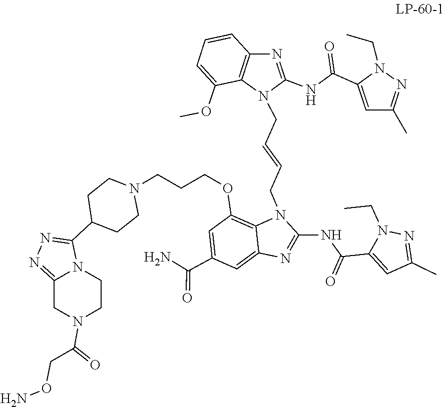 [SMILES: CCn1nc(C)cc1C(=O)Nc1nc2cccc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21]

Non-cleavable LP-60-1 was synthesized and purified by reverse phase preparative HPLC using the same methods as described. LCMS (ESI) m/z 1000.4 (M+H). ¹H NMVR (400 MHz, DMSO) δ 12.89 (s, 1H), 12.72 (s, 1H), 9.36 (s, 1H), 7.96 (s, 1H), 7.67 (s, 1H), 7.38 (s, 1H), 7.29 (d, J=1.4 Hz, 1H), 7.17-7.06 (m, 2H), 6.78 (dd, J=6.4, 2.9 Hz, 1H), 6.51 (d, J=14.9 Hz, 2H), 5.86-5.74 (m, 1H), 4.96-4.86 (m, 4H), 4.80 (s, 2H), 4.70 (s, 2H), 4.52 (q, J=7.1 Hz, 4H), 4.00 (s, 4H), 3.95 (s, 2H), 3.85 (s, 1H), 3.62 (d, J=3.5 Hz, 3H), 3.12 (s, 2H), 2.95 (s, 2H), 2.11 (dd, J=9.7, 3.7 Hz, 6H), 2.04 (s, 2H), 1.94 (s, 4H), 1.36-1.21 (in, 6H).

### Synthesis 3V. LP-49-2

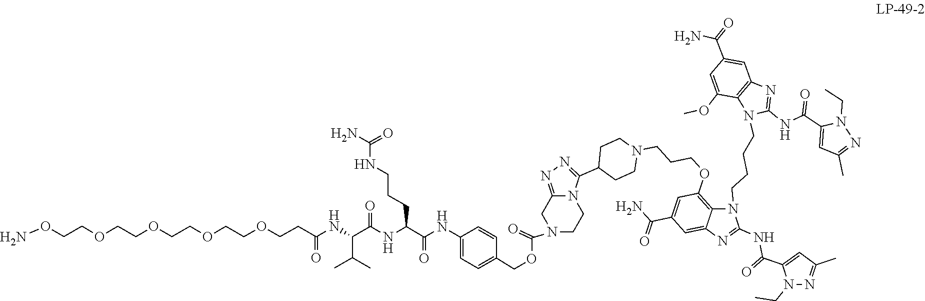 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCN(C(=O)OCc4ccc(NC(=O)[C@H](CCCNC(N)=O)NC(=O)[C@@H](NC(=O)CCOCCOCCOCCOCCON)C(C)C)cc4)C5)CC3)c21]

### 1-(4-(5-carbamoyl-2-(1-ethyl-3-methyl-1H-pyrazole-5-carboxamido)-7-(3-(4-(5,6,7,8-tetrahydro-[1,2,4]triazolo[4,3-a]pyrazin-3-yl)piperidin-1-yl)propoxy)-1H-benzo[d]imidazol-1-yl)butyl)-2-(1-ethyl-3-methyl-1H-pyrazole-5-carboxamido)-7-methoxy-1H-benzo[d]imidazole-5-carboxamide (Compound 49)

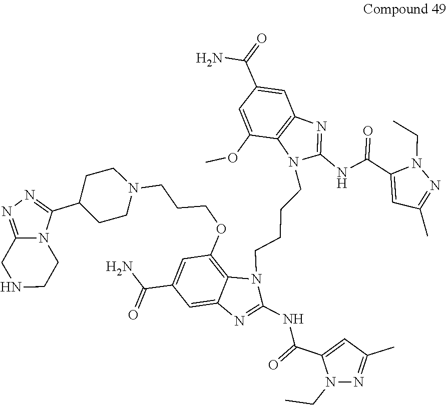 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCNC5)CC3)c21]

A mixture of tert-butyl 3-[1-[3-[6-carbamoyl-3-[4-[5-carbamoyl-2-[(2-ethyl-5-methyl-pyrazole-3-carbonyl)amino]-7-methoxy-benzimidazol-1-yl]butyl]-2-[(2-ethyl-5-methyl-pyrazole-3-carbonyl)amino]benzimidazol-4-yl]oxypropyl]-4-piperidyl]-6,8-dihydro-5H-[1,2,4]triazolo[4,3-a]pyrazine-7-carboxylate (58.6 mg, 0.0547 mmol) in 2 mL of 5% (v/v) trifluoracetic acid in 1,1,1,3,3,3-Hexafluoroisopropanol (HFIP) was stirred at room temperature for 1.5 hour. The reaction mixture was concentrated. The residue was diluted with DMSO (2 mL) and purified by reverse phase preparative HPLC (acetonitrile/water+0.1% TFA) to 1-(4-(5-carbamoyl-2-(1-ethyl-3-methyl-1H-pyrazole-5-carboxamido)-7-(3-(4-(5,6,7,8-tetrahydro-[1,2,4]triazolo[4,3-a]pyrazin-3-yl)piperidin-1-yl)propoxy)-1H-benzo[d]imidazol-1-yl)butyl)-2-(1-ethyl-3-methyl-1H-pyrazole-5-carboxamido)-7-methoxy-1H-benzo[d]imidazole-5-carboxamide (Compound 49) as its TFA salt. (white solid, 40 mg, 61%). LCMS (ESI) m/z 972.4 (M+H). ¹H NMR (400 MHz, DMSO) δ 12.78 (s, 2H), 9.51 (s, 1H), 8.02 (s, 2H), 7.57-7.50 (m, 2H), 7.40 (s, 2H), 7.35-7.29 (m, 2H), 6.60 (dd, J=5.9, 2.5 Hz, 2H), 4.64-4.52 (m, 6H), 4.21-4.14 (m, 4H), 3.83 (d, J=5.8 Hz, 3H), 3.68-3.60 (m, 56H), 2.91 (d, J=11.7 Hz, 2H), 2.14-2.03 (m, 9H), 1.86 (s, 4H), 1.36-1.28 (m, 6H).

### 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl 3-(1-(3-((5-carbamoyl-1-(4-(5-carbamoyl-2-(1-ethyl-3-methyl-1H-pyrazole-5-carboxamido)-7-methoxy-1H-benzo[d]imidazol-1-yl)butyl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazine-7 (8H)-carboxylate

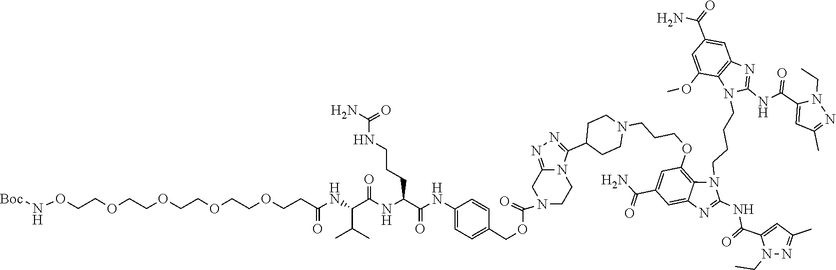 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCN(C(=O)OCc4ccc(NC(=O)[C@H](CCCNC(N)=O)NC(=O)[C@@H](NC(=O)CCOCCOCCOCCOCCONC(=O)OC(C)(C)C)C(C)C)cc4)C5)CC3)c21]

A mixture of [4-[[(2S)-2-[[(2S)-2-[3-[2-[2-[2-[2-(tert-butoxycarbonylamino)oxyethoxy]ethoxy]ethoxy]ethoxy]propanoylamino]-3-methyl-butanoyl]amino]-5-ureido-pentanoyl]amino]phenyl]methyl (4-nitrophenyl) carbonate (Compound 4, 9.6 mg, 0.0106 mmol) and [4-[[(2S)-2-[[(2S)-2-[3-[2-[2-[2-[2-(tert-butoxycarbonylamino)oxyethoxy]ethoxy]ethoxy]ethoxy]propanoylamino]-3-methyl-butanoyl]amino]-5-ureido-pentanoyl]amino]phenyl]methyl (4-nitrophenyl) carbonate (10.8 mg, 0.0111 mmol) in DMF (2 mL) was cooled in an ice bath. DIEA (6.8 mg, 0.010 mL, 0.053 mmol) was then added. The reaction mixture was warmed to room temperature and stirred for 18 hours. The reaction solution was purified by reverse phase preparative HPLC (acetonitrile/water+0.1% formic acid) to afford 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl 3-(1-(3-((5-carbamoyl-1-(4-(5-carbamoyl-2-(1-ethyl-3-methyl-1H-pyrazole-5-carboxamido)-7-methoxy-1H-benzo[d]imidazol-1-yl)butyl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazine-7 (8H)-carboxylate (6.6 mg, 36%). LCMS (ESI) m/z 1741.8 (M+H).

4-((17S,20S)-1-(aminooxy)-17-isopropyl-15,18-dioxo-20-(3-ureidopropyl)-3,6,9,12-tetraoxa-16,19-diazahenicosan-21-amido)benzyl 3-(1-(3-((5-carbamoyl-1-(4-(5-carbamoyl-2-(1-ethyl-3-methyl-1H-pyrazole-5-carboxamido)-7-methoxy-1H-benzo[d]imidazol-1-yl)butyl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazine-7 (8H)-carboxylate (LP-49-2): A mixture of 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl 3-(1-(3-((5-carbamoyl-1-(4-(5-carbamoyl-2-(1-ethyl-3-methyl-1H-pyrazole-5-carboxamido)-7-methoxy-1H-benzo[d]imidazol-1-yl)butyl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazine-7 (8H)-carboxylate (6.6 mg, 0.0038 mmol) in 2 mL of 5% (v/v) trifluoracetic acid in 1,1,1,3,3,3-Hexafluoroisopropanol (HFIP) was stirred at room temperature for 0.5 hour. The reaction mixture was concentrated. The residue was diluted with DMSO (2 mL) and purified by reverse phase preparative HPLC (acetonitrile/water+0.1% TFA) to afford 4-((17S,20S)-1-(aminooxy)-17-isopropyl-15,18-dioxo-20-(3-ureidopropyl)-3,6,9,12-tetraoxa-16,19-diazahenicosan-21-amido)benzyl 3-(1-(3-((5-carbamoyl-1-(4-(5-carbamoyl-2-(1-ethyl-3-methyl-1H-pyrazole-5-carboxamido)-7-methoxy-1H-benzo[d]imidazol-1-yl)butyl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazine-7 (8H)-carboxylate (LP-49-2) as white solid, 7.5 mg, LCMS (ESI) m/z 1641.2 (M+H). ¹H NMR (400 MHz, DMSO) δ 12.79 (s, 2H), 10.51 (s, 3H), 10.02 (s, 1H), 9.34 (s, 1H), 7.58 (dd, J=25.2, 7.8 Hz, 4H), 7.42-7.29 (m, 6H), 6.00 (s, 1H), 5.44 (s, 2H), 4.58 (t, J=8.0 Hz, 4H), 4.35 (d, J=13.9 Hz, 6H), 4.16 (s, 3H), 4.09-4.05 (m, 2H), 3.84 (s, 2H), 3.65-3.62 (m, 2H), 3.59 (dd, J=6.6, 3.2 Hz, 1H), 3.55-3.42 (m, 13H), 2.94 (q, J=16.7 Hz, 2H), 2.11 (d, J=2.9 Hz, 5H), 2.05 (s, 6H), 1.86 (s, 4H), 1.32 (q, J=7.3 Hz, 6H), 0.84 (dd, J=13.0, 6.8 Hz, 6H).

### Synthesis 3W. LP-57-1

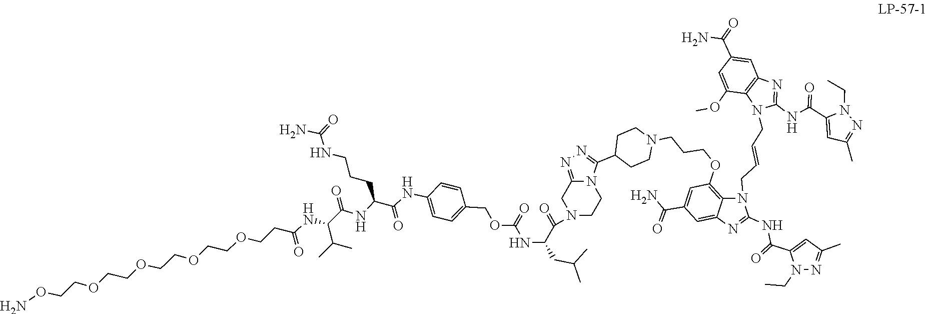 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)OCc4ccc(NC(=O)[C@H](CCCNC(N)=O)NC(=O)[C@@H](NC(=O)CCOCCOCCOCCOCCON)C(C)C)cc4)C5)CC3)c21]

### tert-butyl (S,E)-(1-(3-(1-(3-((5-carbamoyl-1-(4-(5-carbamoyl-7-methoxy-2-(3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate

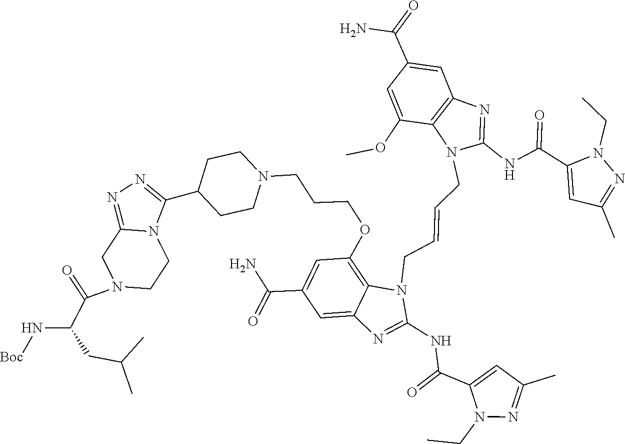 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)OC(C)(C)C)C5)CC3)c21]

To a mixture of 1-[(E)-4-[5-carbamoyl-2-[(2-ethyl-5-methyl-pyrazole-3-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]but-2-enyl]-2-[(2-ethyl-5-methyl-pyrazole-3-carbonyl)amino]-7-methoxy-benzimidazole-5-carboxamide; 2,2,2-trifluoroacetic acid (Compound 4, 17.1 mg, 0.014 mmol), (2S)-2-(tert-butoxycarbonylamino)-4-methyl-pentanoic acid (4.0 mg, 0.0171 mmol), DIEA (9.2 mg, 0.012 mL, 0.071 mmol) in DMF (2 mL) was added HBTU (7.1 mg, 0.0187 mmol). The reaction was stirred for 15 minutes. The reaction solution was purified by reverse phase preparative HPLC (acetonitrile/water+0.100 formic acid) to afford tert-butyl (S,E)-(1-(3-(1-(3-((5-carbamoyl-1-(4-(5-carbamoyl-7-methoxy-2-(3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (11.8 mg, 70%). LCMS (ESI) m/z 1183.6 (M+H).

### (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide (Compound 57)

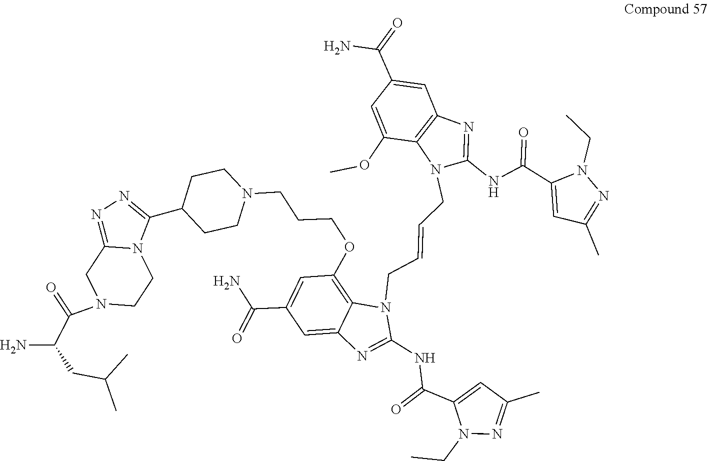 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@@H](N)CC(C)C)C5)CC3)c21]

To a mixture of tert-butyl (S,E)-(1-(3-(1-(3-((5-carbamoyl-1-(4-(5-carbamoyl-7-methoxy-2-(3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (11.8 mg, 0.010 mmol) in MeOH (2 mL) was added hydrogen chloride, 4.0 M in 1,4-dioxane (0.15 mL, 0.60 mmol). The reaction mixture was stirred at room temperature for 4 hours, then concentrated. The residue was dissolved in DMSO (2 mL) and 28-30% NH₃·H₂O (0.1 mL) then purified by reverse phase preparative HPLC (acetonitrile/water+0.1% 28-30% NH₃·H₂O) to afford (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide (Compound 57), (white solid, 9.2 mg). LCMS (ESI) m/z 1083.6 (M+H). ¹H NMR (400 MHz, DMSO) δ 7.94 (s, 2H), 7.65 (d, J=6.5 Hz, 2H), 7.29 (d, J=14.9 Hz, 4H), 6.56 (s, 1H), 6.50 (d, J=2.8 Hz, 2H), 5.89-5.81 (m, 2H), 4.94 (s, 2H), 4.90 (s, 3H), 4.52 (dt, J=21.2, 7.2 Hz, 3H), 4.01 (s, 2H), 3.95 (s, 5H), 3.72 (s, 4H), 2.76 (s, 2H), 2.29 (s, 4H), 2.11 (d, J=17.6 Hz, 7H), 1.80 (d, J=11.3 Hz, 4H), 1.72 (s, 3H), 1.67 (s, 6H), 1.28 (dt, J=17.9, 7.1 Hz, 8H), 0.91 (d, J=6.6 Hz, 6H).

### 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((S)-1-(3-(1-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate

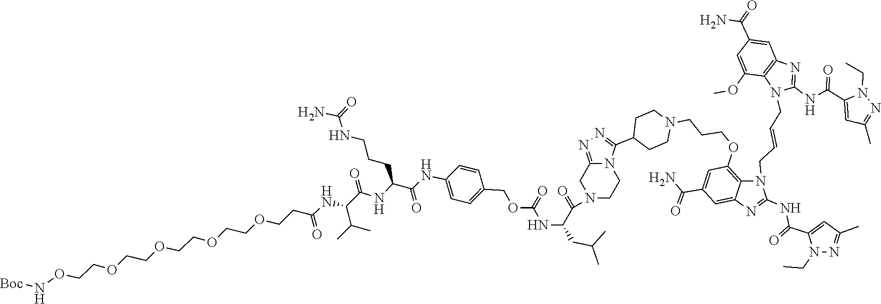 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)OCc4ccc(NC(=O)[C@H](CCCNC(N)=O)NC(=O)[C@@H](NC(=O)CCOCCOCCOCCOCCONC(=O)OC(C)(C)C)C(C)C)cc4)C5)CC3)c21]

A mixture of (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide (Compound 57, 24 mg, 0.0106 mmol) and [4-[[(2S)-2-[[(2S)-2-[3-[2-[2-[2-[2-(tert-butoxycarbonylamino)oxyethoxy]ethoxy]ethoxy]ethoxy]propanoylamino]-3-methyl-butanoyl]amino]-5-ureido-pentanoyl]amino]phenyl]methyl (4-nitrophenyl) carbonate (22.1 mg, 0.0244 mmol) in DMF (2 mL) was cooled in an ice bath. DIEA (14.3 mg, 0.019 mL, 0.111 mmol) was then added. The reaction mixture was warmed to room temperature and stirred for 18 hours. The reaction solution was purified by reverse phase preparative HPLC (acetonitrile/water+0.1% formic acid) to afford 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((S)-1-(3-(1-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (28 mg, 68%). LCMS (ESI) m/z 1853.1 (M+H).

4-((17S,20S)-1-(aminooxy)-17-isopropyl-15,18-dioxo-20-(3-ureidopropyl)-3,6,9,12-tetraoxa-16,19-diazahenicosan-21-amido)benzyl ((S)-1-(3-(1-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (LP-57-1): A mixture of 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((S)-1-(3-(1-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (60 mg, 0.0324 mmol) in 2 mL of 5% (v/v) trifluoracetic acid in 1,1,1,3,3,3-Hexafluoroisopropanol (HFIP) was stirred at room temperature for 0.5 hour. The reaction mixture was concentrated. The residue was diluted with DMSO (2 mL) and purified by reverse phase preparative HPLC (acetonitrile/water+0.1% TFA) to afford 4-((17S,20S)-1-(aminooxy)-17-isopropyl-15,18-dioxo-20-(3-ureidopropyl)-3,6,9,12-tetraoxa-16,19-diazahenicosan-2i-amido)benzyl ((S)-1-(3-(1-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (LP-57-1) as its TFA salt. (white solid, 60 mg, 88%). LCMS (ESI) m/z 1752.4 (M+H). ¹H NMR (400 MHz, DMSO) δ 12.86 (s, 2H), 11.03 (s, 1H), 9.96 (s, 1H), 8.16-8.05 (m, 2H), 7.96 (d, J=18.0 Hz, 2H), 7.85 (d, J=8.7 Hz, 1H), 7.67 (d, J=3.8 Hz, 2H), 7.58 (d, J=8.3 Hz, 2H), 7.38-7.24 (m, 6H), 6.95-6.89 (m, 1H), 6.53 (d, J=6.3 Hz, 2H), 5.99 (s, 1H), 5.78 (q, J=15.8 Hz, 2H), 5.41 (s, 2H), 4.95 (dd, J=24.7, 12.7 Hz, 7H), 4.71 (d, J=16.9 Hz, 1H), 4.58-4.48 (m, 3H), 4.38 (q, J=7.3 Hz, 1H), 4.23 (dd, J=8.7, 6.6 Hz, 1H), 4.08-4.01 (m, 2H), 3.71 (s, 3H), 3.66-3.56 (m, 3H), 2.94 (s, 6H), 2.49-2.34 (m, 2H), 2.12 (d, J=10.9 Hz, 5H), 2.01-1.90 (m, 2H), 1.28 (m, 6H), 0.94-0.80 (m, 12H).

### Synthesis 3X. LP-66-1

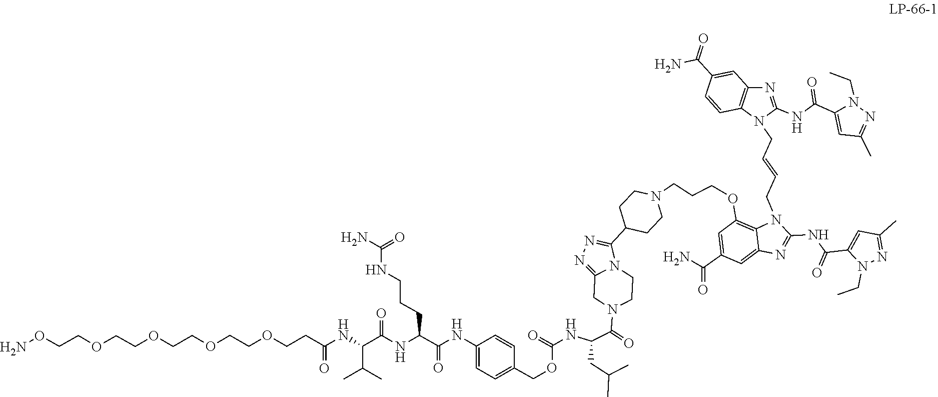 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)OCc4ccc(NC(=O)[C@H](CCCNC(N)=O)NC(=O)[C@@H](NC(=O)CCOCCOCCOCCOCCON)C(C)C)cc4)C5)CC3)c21]

### tert-butyl (S,E)-(1-(3-(1-(3-((5-carbamoyl-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate

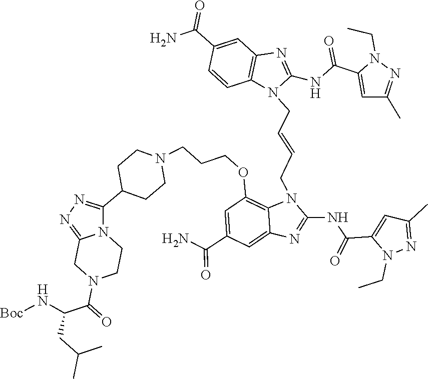 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)OC(C)(C)C)C5)CC3)c21]

To a mixture of (E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-(4-(5,6,7,8-tetrahydro-[1,2,4]triazolo[4,3-a]pyrazin-3-yl)piperidin-1-yl)propoxy)-1H-benzo[d]imidazole-5-carboxamide (Compound 64, 16.3 mg, 0.0173 mmol), (2S)-2-(tert-butoxycarbonylamino)-4-methyl-pentanoic acid (4.81 mg, 0.0208 mmol), DIEA (11.2 mg, 0.015 mL, 0.0867 mmol) in DMF (2 mL) was added HBTU (8.6 mg, 0.023 mmol). The reaction was stirred for 15 minutes. The reaction solution was purified by reverse phase preparative HPLC (acetonitrile/water+0.1% formic acid) to afford tert-butyl (S,E)-(1-(3-(1-(3-((5-carbamoyl-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (10.9 mg, 55%). LCMS (ESI) m/z 1153.6 (M+H).

### (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazole-5-carboxamide (Compound 66)

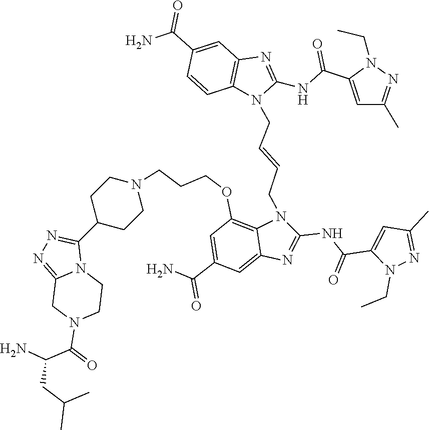 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)[C@@H](N)CC(C)C)C5)CC3)c21]

To a mixture of tert-butyl (S,E)-(1-(3-(1-(3-((5-carbamoyl-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (10.9 mg, 0.0095 mmol) in MeOH (2 mL) was added hydrogen chloride, 4.0 M in 1,4-dioxane (0.14 mL, 0.567 mmol). The reaction mixture was stirred at room temperature for 4 hours, then concentrated. The residue was dissolved in DMSO (2 mL) and 28-30% NH₃·H₂O (0.1 mL) then purified by reverse phase preparative HPLC (acetonitrile/water+0.1% 28-30% NH₃·H₂O) to afford (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazole-5-carboxamide (Compound 66), (white solid, 4.2 mg, 42%). LCMS (ESI) m/z 1053.6 (M+H). ¹H NMR (400 MHz, DMSO) δ 7.99 (s, 1H), 7.93 (s, 2H), 7.71 (d, J=8.4 Hz, 1H), 7.64 (s, 1H), 7.42 (d, J=8.2 Hz, 1H), 7.29 (s, 2H), 6.54 (d, J=10.3 Hz, 2H), 6.48 (s, 1H), 5.99 (s, 1H), 4.96 (s, 2H), 4.82 (s, 2H), 4.54 (dd, J=17.3, 7.4 Hz, 4H), 4.01 (s, 3H), 2.79 (s, 1H), 2.12 (d, J=12.5 Hz, 6H), 1.91 (s, 2H), 1.53 (s, 1H), 1.28 (dt, J=14.4, 7.1 Hz, 6H), 0.99-0.88 (m, 6H).

### 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((S)-1-(3-(1-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate

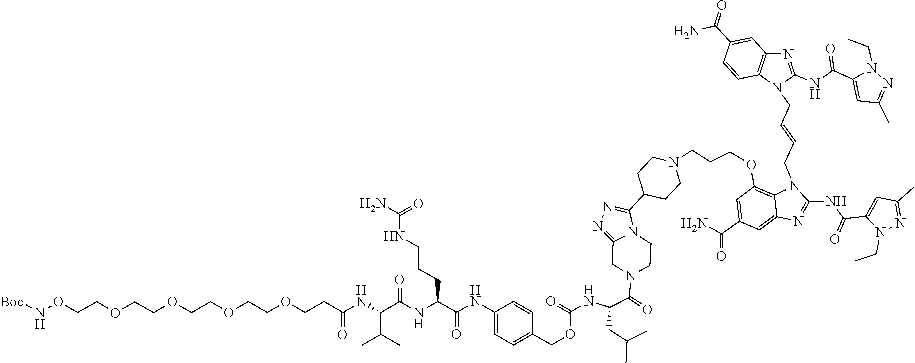 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)OCc4ccc(NC(=O)[C@H](CCCNC(N)=O)NC(=O)[C@@H](NC(=O)CCOCCOCCOCCOCCONC(=O)OC(C)(C)C)C(C)C)cc4)C5)CC3)c21]

A mixture of (E)-7-(3-(4-(7-(L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazole-5-carboxamide (Compound 66, 28.6 mg, 0.0272 mmol) and [4-[[(2S)-2-[[(2S)-2-[3-[2-[2-[2-[2-(tert-butoxycarbonylamino)oxyethoxy]ethoxy]ethoxy]ethoxy]propanoylamino]-3-methyl-butanoyl]amino]-5-ureido-pentanoyl]amino]phenyl]methyl (4-nitrophenyl) carbonate (27.1 mg, 0.0299 mmol) in DMF (2 mL) was cooled in an ice bath. DIEA (17.6 mg, 0.024 mL, 0.136 mmol) was then added. The reaction mixture was warmed to room temperature and stirred for 18 hours. The reaction solution was purified by reverse phase preparative HPLC (acetonitrile/water+0.1% formic acid) to afford 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((S)-1-(3-(1-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (22 mg, 44%). LCMS (ESI) m/z 1822.7 (M+H).

4-((17S,20S)-1-(aminooxy)-17-isopropyl-15,18-dioxo-20-(3-ureidopropyl)-3,6,9,12-tetraoxa-16,19-diazahenicosan-21-amido)benzyl ((S)-1-(3-(1-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (LP-66-1): A mixture of 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((S)-1-(3-(1-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate (22 mg, 0.0121 mmol) in 2 mL of 5% (v/v) trifluoracetic acid in 1,1,1,3,3,3-Hexafluoroisopropanol (HFIP) was stirred at room temperature for 0.5 hour. The reaction mixture was concentrated. The residue was diluted with DMSO (2 mL) and purified by reverse phase preparative HPLC (acetonitrile/water+0.1% TFA) to afford 4-((17S,20S)-1-(aminooxy)-17-isopropyl-15,18-dioxo-20-(3-ureidopropyl)-3,6,9,12-tetraoxa-16,19-diazahenicosan-21-amido)benzyl ((S)-1-(3-(1-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)carbamate LP-66-1 as its TFA salt. (white solid, 21 mg, 87%). LCMS (ESI) m/z 1722.6 (M+H). ¹H NMR (400 MHz, DMSO) δ 12.84 (s, 3H), 10.51 (s, 4H), 9.98 (s, 1H), 9.34 (s, 1H), 8.11 (d, J=7.5 Hz, 1H), 8.00 (s, 1H), 7.95 (s, 3H), 7.86 (d, J=8.6 Hz, 1H), 7.76-7.65 (m, 2H), 7.58 (d, J=8.3 Hz, 2H), 7.42 (d, J=8.3 Hz, 1H), 7.31 (dd, J=26.1, 9.8 Hz, 7H), 6.54 (d, J=2.1 Hz, 2H), 5.98 (d, J=7.5 Hz, 2H), 5.93 (s, 1H), 4.98 (d, J=12.6 Hz, 1H), 4.94 (s, 5H), 4.82 (s, 3H), 4.72 (d, J=16.8 Hz, 1H), 4.53 (dd, J=14.6, 7.2 Hz, 3H), 4.38 (d, J=6.1 Hz, 1H), 4.27-4.19 (m, 1H), 4.07 (m, 4H), 3.67-3.56 (m, 4H), 3.55-3.52 (m, 4H), 3.49 (d, J=6.5 Hz, 13H), 3.35 (s, 1H), 3.01 (s, 1H), 2.95 (s, 5H), 2.49-2.33 (m, 2H), 2.12 (d, J=8.4 Hz, 6H), 2.09 (s, 5H), 2.01-1.93 (m, 2H), 1.67 (s, 2H), 1.34-1.22 (m, 6H), 0.94-0.79 (m, 12H)

### Synthesis 3Y. LP-25-2

LP-25-2 was synthesized as described in Scheme 25.

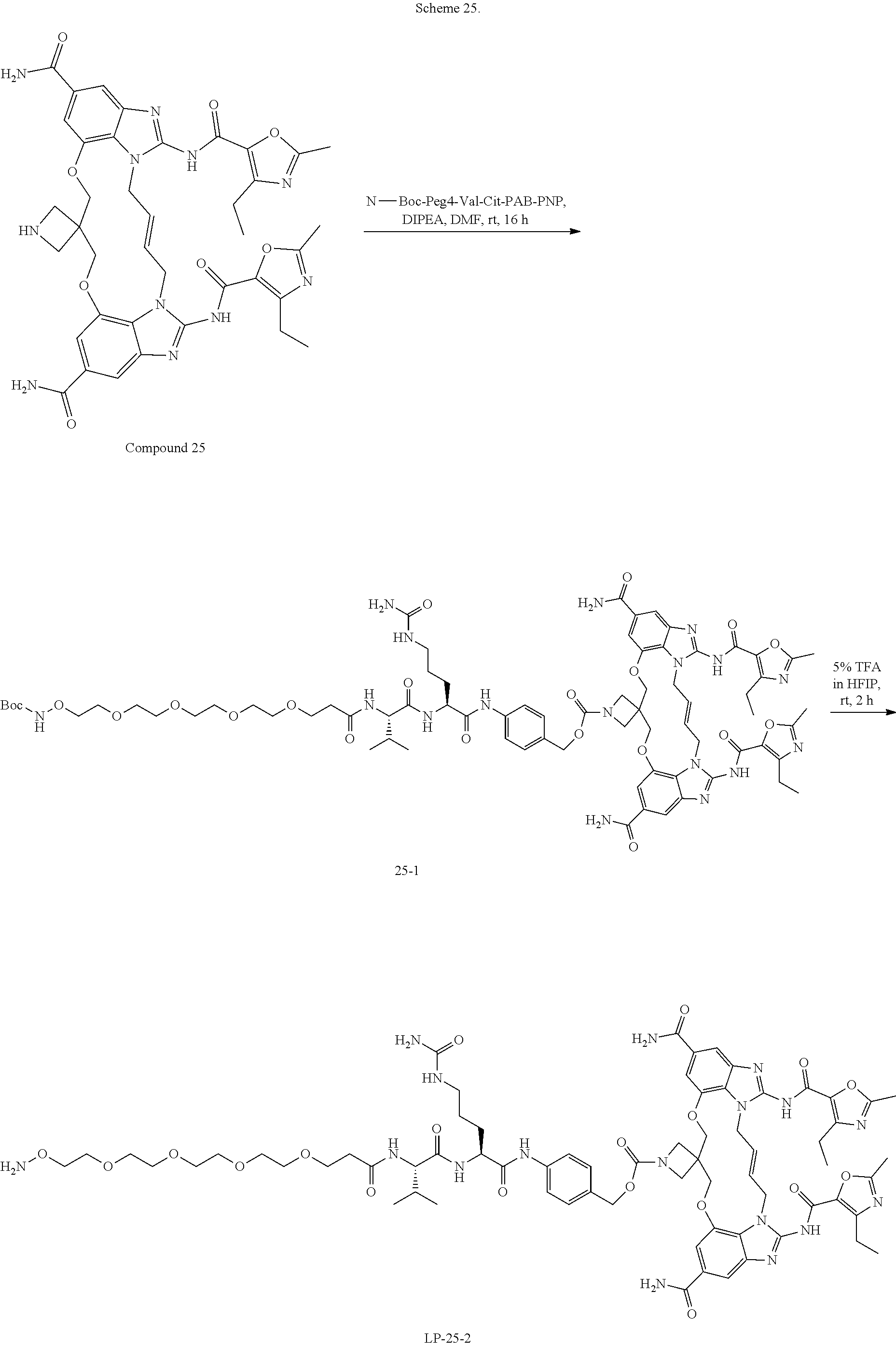

Synthesis of [4-[[(2S)-2-[[(2S)-2-[3-[2-[2-[2-[2-(tert-butoxycarbonylamino)oxyethoxy]ethoxy]ethoxy]ethoxy]propanoylamino]-3-methyl-butanoyl]amino]-5-ureido-pentanoyl]amino]phenyl]methyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (25-1): To a stirred solution of (3E)-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-11,21-dicarboxamide (Compound 25, 21. mg, 0.03 mmol) in DMF (0.5000 mL) at room temperature was added DIPEA (0.02 mL, 0.13 mmol) followed by the addition of [4-[[(2S)-2-[[(2S)-2-[3-[2-[2-[2-[2-(tert-butoxycarbonylamino)oxyethoxy]ethoxy]ethoxy]ethoxy]propanoylamino]-3-methyl-butanoyl]amino]-5-ureido-pentanoyl]amino]phenyl]methyl (4-nitrophenyl) carbonate (24 mg, 0.03 mmol). The solution was stirred at room temperature for 16 hours. Reaction mixture was purified by prep-HPLC (Column: Phenomenex kinetex 5 μm C₁₈ 100 Å, 250×21.2 mm and using 5-50% ACN: 1% TFA in water as eluent, Flow rate: 21.2 mL/min, run time: 30 mins to afford [4-[[(2S)-2-[[(2S)-2-[3-[2-[2-[2-[2-(tert-butoxycarbonylamino)oxyethoxy]ethoxy]ethoxy]ethoxy]propanoylamino]-3-methyl-butanoyl]amino]-5-ureido-pentanoyl]amino]phenyl]methyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (25-1, 23 mg, 0.015 mmol) as a white solid LCMS (ESI) m/z 1560.70 (M+H).

Synthesis of [4-[[(2S)-2-[[(2S)-2-[3-[2-[2-[2-(2-aminooxyethoxy)ethoxy]ethoxy]ethoxy]propanoylamino]-3-methyl-butanoyl]amino]-5-ureido-pentanoyl]amino]phenyl]methyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (LP-25-2):

To a stirred solution of [4-[[(2S)-2-[[(2S)-2-[3-[2-[2-[2-[2-(tert-butoxycarbonylamino)oxyethoxy]ethoxy]ethoxy]ethoxy]propanoylamino]-3-methyl-butanoyl]amino]-5-ureido-pentanoyl]amino]phenyl]methyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate (25-1, 23 mg, 0.01 mmol) in HFIP (0.5 mL) at room temperature was added 5% TFA in HFIP (1.13 mL, 0.74 mmol). The solution was stirred at room temperature for 2 hours. Reaction mixture was concentrated under reduced pressure and crude was purified by reverse phase preparative HPLC (Column: Phenomenex kinetex 5 μm C18 100 Å, 250×21.2 mm and using 5-50% ACN: 1% TFA in water as eluent, Flow rate: 21.2 mL/min, run time: 30 mins to give [4-[[(2S)-2-[[(2S)-2-[3-[2-[2-[2-(2-aminooxyethoxy)ethoxy]ethoxy]ethoxy]propanoylamino]-3-methyl-butanoyl]amino]-5-ureido-pentanoyl]amino]phenyl]methyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13 (27), 19 (26), 20,22,24-nonaene-16,3′-azetidine]-1′-carboxylate LP-25-2 (14.1 mg) as a white solid. ¹H NMR (400 MHz, DMSO) δ 10.03 (s, 1H), 8.14 (d, J=7.4 Hz, 1H), 8.00 (s, 2H), 7.89 (d, J=8.7 Hz, 1H), 7.84 (s, 2H), 7.77 (d, J=1.2 Hz, 2H), 7.61 (d, J=8.5 Hz, 2H), 7.45 (s, 2H), 7.35 (d, J=8.4 Hz, 2H), 6.03 (s, 1H), 5.33 (s, 2H), 5.05 (s, 2H), 4.80 (s, 4H), 4.55 (s, 4H), 4.39 (q, J=7.5 Hz, 1H), 4.24 (dd, J=8.7, 6.6 Hz, 1H), 4.11-4.05 (m, 2H), 3.97 (d, J=16.7 Hz, 4H), 3.66-3.62 (m, 6H), 2.77 (q, J=7.5 Hz, 4H), 2.37 (s, 6H), 1.98 (dt, J=13.5, 6.8 Hz, 1H), 1.69 (t, J=7.7 Hz, 1H), 1.60 (dd, J=9.3, 4.9 Hz, 1H), 1.41 (dt, J=25.0, 5.5 Hz, 3H), 0.96 (t, J=7.5 Hz, 6H), 0.85 (dd, J=13.2, 6.8 Hz, 6H).

### Synthesis 3Z. LP-2-4

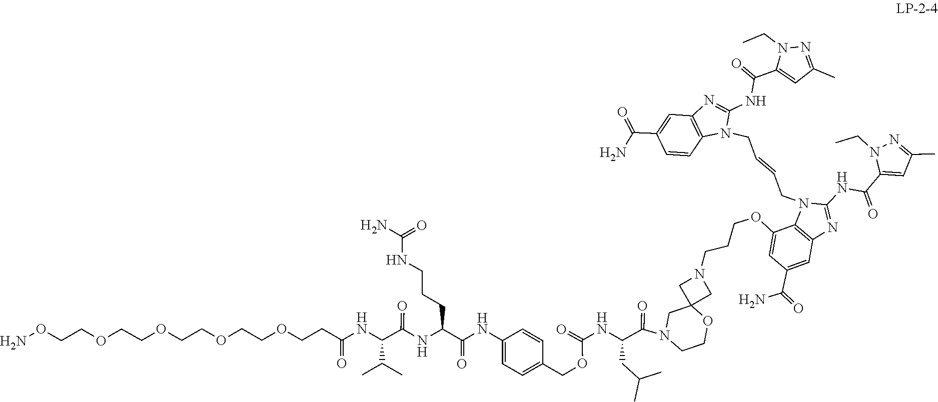 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@H](CC(C)C)NC(=O)OCc3ccc(NC(=O)[C@H](CCCNC(N)=O)NC(=O)[C@@H](NC(=O)CCOCCOCCOCCOCCON)C(C)C)cc3)CCO4)c21]

### 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((S)1(2-3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)-5-oxa-2,8-diazaspiro[3.5]nonan-8-yl)-4-methyl-1-oxopentan-2-yl)carbamate

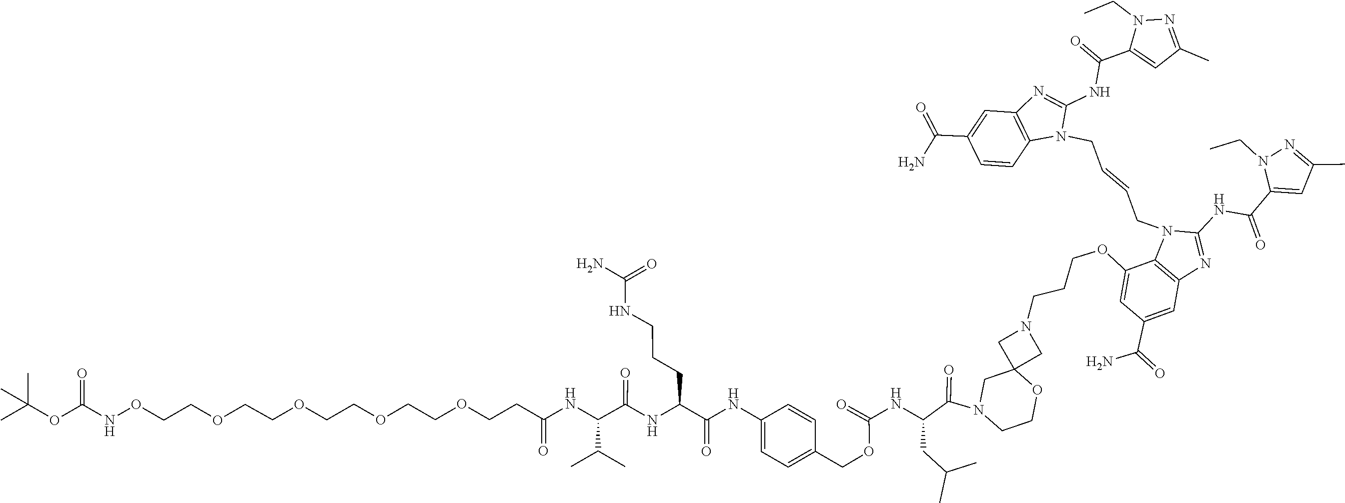 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@H](CC(C)C)NC(=O)OCc3ccc(NC(=O)[C@H](CCCNC(N)=O)NC(=O)[C@@H](NC(=O)CCOCCOCCOCCOCCONC(=O)OC(C)(C)C)C(C)C)cc3)CCO4)c21]

To a DMF (1 mL) solution of (E)-7-(3-(8-(L-leucyl)-5-oxa-2,8-diazaspiro[3.5]nonan-2-yl)propoxy)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazole-5-carboxamide (Compound 2, 77 mg, 0.08 mmol) and DIEA (0.138 mL, 0.80 mmol) was added DMF (1 mL) solution of tert-butyl (((6S,9S)-1-amino-9-isopropyl-6-((4-((((4-nitrophenoxy)carbonyl)oxy)methyl)phenyl)carbamoyl)-1,8,11-trioxo-14,17,20,23-tetraoxa-2,7,10-triazapentacosan-25-yl)oxy)carbamate (79.0 mg, 0.087 mmol) dropwise at 0° C., reaction mixture was stirred at 0° C. for 15 mins, then gradually warmed to room temperature and stirred overnight. The reaction solution was purified by reverse phase preparative HPLC (acetonitrile/water+0.1% formic acid) to afford 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((S)-1-(2-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)-5-oxa-2,8-diazaspiro[3.5]nonan-8-yl)-4-methyl-1-oxopentan-2-yl)carbamate as a white solid (82 mg). LCMS (ESI) m/z 1742.9 (M+H).

4-((17S,20S)-1-(aminooxy)-17-isopropyl-15,18-dioxo-20-(3-ureidopropyl)-3,6,9,12-tetraoxa-16,19-diazahenicosan-21-amido)benzyl ((S)-1-(2-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)-5-oxa-2,8-diazaspiro[3.5]nonan-8-yl)-4-methyl-1-oxopentan-2-yl)carbamate (LP-2-4): A mixture of 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((S)-1-(2-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)-5-oxa-2,8-diazaspiro[3.5]nonan-8-yl)-4-methyl-1-oxopentan-2-yl)carbamate (82.0 mg, 0.047 mmol) in 2 mL of 5% (v/v) trifluoracetic acid in 1,1,1,3,3,3-Hexafluoroisopropanol (HFIP) was stirred at room temperature for 1 hour. The reaction mixture was concentrated. The residue was diluted with DMSO (2 mL) and purified by reverse phase preparative HPLC (acetonitrile/water+0.1% TFA) to afford 4-((17S,20S)-1-(aminooxy)-17-isopropyl-15,18-dioxo-20-(3-ureidopropyl)-3,6,9,12-tetraoxa-16,19-diazahenicosan-21-amido)benzyl ((S)-1-(2-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)-5-oxa-2,8-diazaspiro[3.5]nonan-8-yl)-4-methyl-1-oxopentan-2-yl)carbamate (LP-2-4) as white solid, 57 mg, 74%). LCMS (ESI) m/z 1642.8 (M+H). ¹H NMR (400 MHz, DMSO) δ 12.84 (s, 2H), 10.40 (s, 3H), 7.96 (d, J=16.1 Hz, 3H), 7.73-7.64 (m, 2H), 7.59 (d, J=8.2 Hz, 3H), 7.38 (d, J=8.6 Hz, 3H), 7.27 (d, J=8.2 Hz, 2H), 6.53 (d, J=12.8 Hz, 2H), 5.98 (d, J=17.3 Hz, 2H), 4.94 (d, J=13.6 Hz, 4H), 4.53 (d, J=7.3 Hz, 4H), 4.41-4.35 (m, 3H), 4.09-4.03 (m, 3H), 3.85-3.76 (m, 56H), 3.61 (dt, J=17.1, 4.8 Hz, 5H), 3.55-3.46 (m, 12H), 3.17 (s, 14H), 2.10 (d, J=17.0 Hz, 11H), 1.82 (s, 2H), 1.27 (td, J=7.1, 2.9 Hz, 6H), 0.89-0.79 (m, 12H).

### Synthesis 3AA. LP-63-1

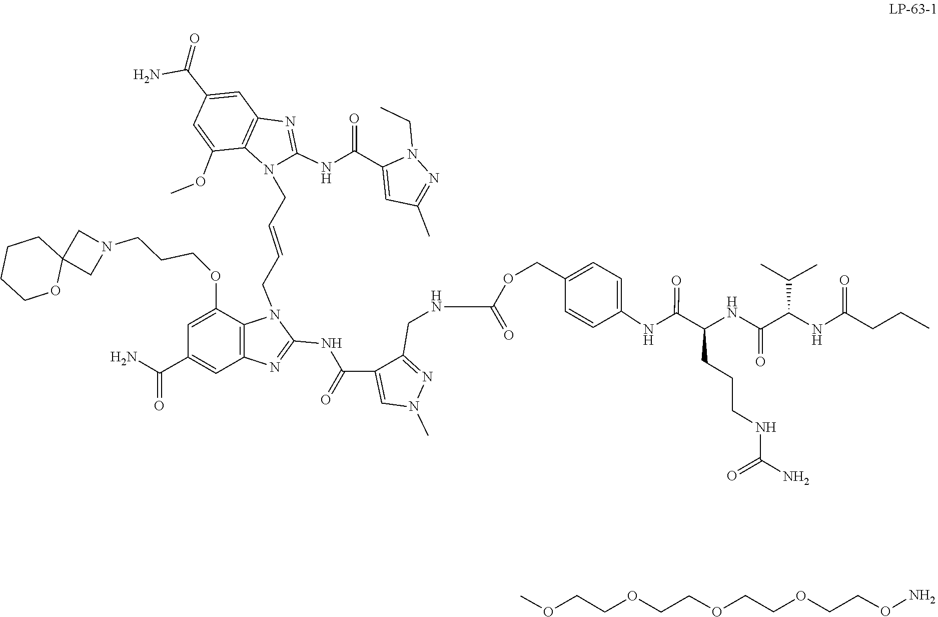

### 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((4-((7-(3-(5-oxa-2-azaspiro[3.5]nonan-2-yl)propoxy)-5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate

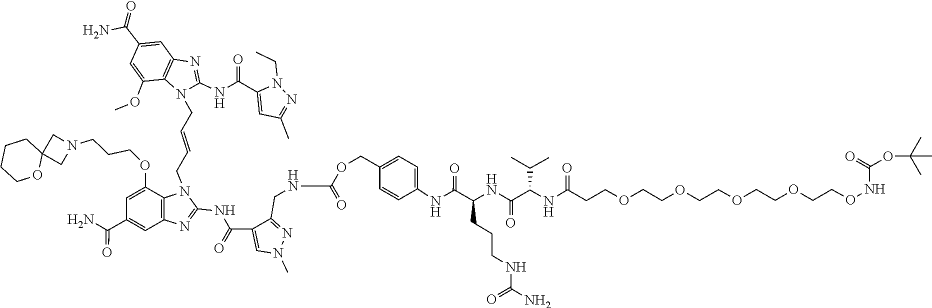 [SMILES: CCn1nc(C)cc1C(=O)Nc1nc2cc(C(N)=O)cc(OC)c2n1C/C=C/Cn1c(NC(=O)c2cn(C)nc2CNC(=O)OCc2ccc(NC(=O)[C@H](CCCNC(N)=O)NC(=O)[C@@H](NC(=O)CCOCCOCCOCCOCCONC(=O)OC(C)(C)C)C(C)C)cc2)nc2cc(C(N)=O)cc(OCCCN3CC4(CCCCO4)C3)c21]

To a DMF (1 mL) solution of (E)-7-(3-(5-oxa-2-azaspiro[3.5]nonan-2-yl)propoxy)-2-(3-(aminomethyl)-1-methyl-1H-pyrazole-4-carboxamido)-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazole-5-carboxamide (Compound 63, 20 mg) and DIEA (0.039 mL, 0.22 mmol) was added DMF (0.5 mL) solution of tert-butyl (((6S,9S)-1-amino-9-isopropyl-6((4((((4nitrophenoxy)carbonyl)oxy)methyl) phenyl)carbamoyl)-1,8,11-trioxo-14,17,20,23-tetraoxa-2,7,10-triazapentacosan-25-yl)oxy)carbamate (20.4 mg, 0.022 mmol) dropwise at 0° C., reaction mixture was stirred at 0° C. for 15 mins, then gradually warmed to room temperature and stirred overnight. The reaction solution was purified by reverse phase preparative HPLC (acetonitrile/water+0.1% formic acid) to afford 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((4-((7-(3-(5-oxa-2-azaspiro[3.5]nonan-2-yl)propoxy)-5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate as a white solid (24 mg). LCMS (ESI) m/z 1659.8 (M+H).

4-((17S,20S)-1-(aminooxy)-17-isopropyl-15,18-dioxo-20-(3-ureidopropyl)-3,6,9,12-tetraoxa-16,19-diazahenicosan-21-amido)benzyl ((4-((7-(3-(5-oxa-2-azaspiro[3.5]nonan-2-yl)propoxy)-5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate: A mixture of 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((4-((7-(3-(5-oxa-2-azaspiro[3.5]nonan-2-yl)propoxy)-5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate (24.0 mg, 0.022 mmol) in 2 mL of 5% (v/v) trifluoracetic acid in 1,1,1,3,3,3-Hexafluoroisopropanol (HFIP) was stirred at room temperature for 1 hour. The reaction mixture was concentrated. The residue was diluted with DMSO (2 mL) and purified by reverse phase preparative HPLC (acetonitrile/water+0.1% TFA) to afford 4-((17S,20S)-1-(aminooxy)-17-isopropyl-15,18-dioxo-20-(3-ureidopropyl)-3,6,9,12-tetraoxa-16,19-diazahenicosan-21-amido)benzyl ((4-((7-(3-(5-oxa-2-azaspiro[3.5]nonan-2-yl)propoxy)-5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate (LP-63-1) as its TFA salt. (white solid, 13.0 mg, 58%). LCMS (ESI) m/z 1559.8 (M+H). ¹H NMR (400 MHz, DMSO) δ 12.88 (s, 1H), 10.00 (s, 1H), 8.16-8.08 (m, 2H), 7.99 (s, 1H), 7.96-7.85 (m, 2H), 7.66 (s, 1H), 7.59 (d, J=8.2 Hz, 2H), 7.41 (s, 1H), 7.36 (s, 1H), 7.27 (d, J=16.6 Hz, 5H), 6.54 (s, 1H), 5.99 (s, 1H), 5.81 (s, 2H), 5.43 (s, 2H), 4.95 (s, 3H), 4.88 (s, 2H), 4.53 (s, 4H), 4.28-4.20 (m, 1H), 4.05 (s, 2H), 3.93 (s, 3H), 3.79 (s, 2H), 3.67-3.57 (m, 1H), 3.55-3.46 (m, 11H), 3.20 (s, 2H), 2.95 (s, 1H), 2.39 (dd, J=13.6, 6.8 Hz, 1H), 2.09 (d, J=10.4 Hz, 3H), 1.65 (s, 6H), 1.41 (s, 7H), 1.33-1.22 (m, 3H), 0.85 (dd, J=13.2, 6.8 Hz, 6H).

### Synthesis 3BB. LP-57-2

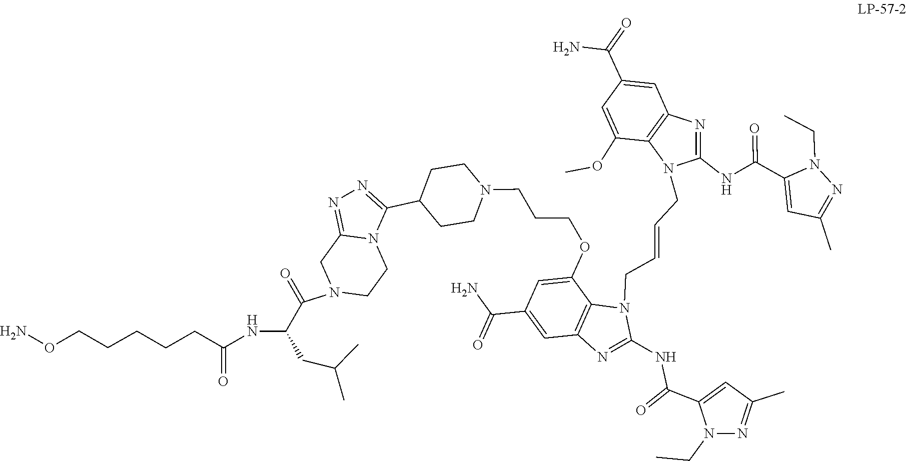 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)CCCCCON)C5)CC3)c21]

### tert-butyl (S,E)-((6-((1-(3-(1-(3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)amino)-6-oxohexyl)oxy)carbamate

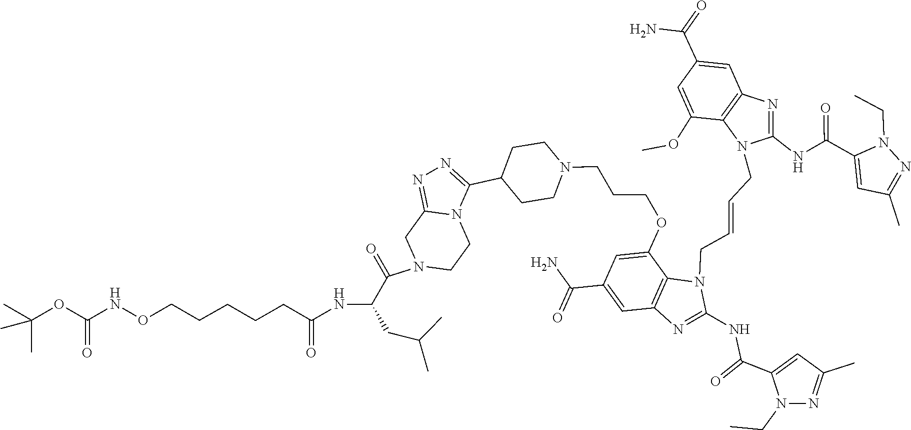 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)CCCCCONC(=O)OC(C)(C)C)C5)CC3)c21]

A solution of 6-(tert-butoxycarbonylamino)oxyhexanoic acid (47.0 mg, 0.189 mmol), HBTU (72.0 mg, 0.189 mmol) and DIEA (0.11 mL, 0.63 mmol in DMF (1.0 mL) was stirred for 10 min and the mixture was then added dropwise to DMF (1.0 mL) solution of (S,E)-7-(3-(4-(7-(2-(12-azaneyl)-4-methylpentanoyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide HCl salt (Compound 57, 141 mg, 0.126 mmol). The reaction mixture was stirred at room temperature for 2 hours, then purified by reverse phase preparative HPLC (acetonitrile/water+0.1% formic acid) to afford tert-butyl (S,E)-((6-((1-(3-(1-(3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)amino)-6-oxohexyl)oxy)carbamate as a white solid (77 mg, 46%). LCMS (ESI) m/z 1312.7 (M+H).

(E)-7-(3-(4-(7-((6-(aminooxy)hexanoyl)-L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide (LP-57-2): A mixture of tert-butyl (S,E)-((6-((1-(3-(1-(3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)amino)-6-oxohexyl)oxy)carbamate (77.0 mg, 0.058 mmol) in 2 mL of 5% (v/v) trifluoracetic acid in 1,1,1,3,3,3-hexafluoroisopropanol (HFIP) was stirred at room temperature for 1 hour. The reaction mixture was concentrated. The residue was diluted with DMSO (2 mL) and purified by reverse phase preparative HPLC (acetonitrile/water +0.1% TFA) to afford (E)-7-(3-(4-(7-((6-(aminooxy)hexanoyl)-L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide (LP-57-2) as its TFA salt. ¹H NMR (400 MHz, DMSO) δ 7.97 (s, OH), 7.68 (d, J=4.5 Hz, 1H), 7.40 (s, 1H), 7.32 (d, J=12.4 Hz, 1H), 6.54 (d, J=6.9 Hz, 1H), 4.92 (d, J=13.2 Hz, 2H), 4.53 (dd, J=15.5, 7.7 Hz, 2H), 4.02 (s, 3H), 3.97-3.87 (m, 1H), 3.71 (s, 1H), 3.47 (s, 5H), 3.11 (s, 1H), 2.89 (s, 1H), 2.12 (d, J=12.0 Hz, 3H), 1.92 (s, 2H), 1.55 (s, 3H), 1.28 (dt, J=15.9, 7.0 Hz, 3H), 0.94-0.87 (m, 3H).

### Synthesis 3CC. General Procedure for Additional Catabolites

To a stirred solution of aminooxy payloads (1 eq) in DMSO (20 mL/mmol) at room temperature was added the solution of (2S)-3-(4-acetylphenyl)-2-amino-propanoic acid (1.5 eq) in DMSO (20 mL/mmol). The reaction mixture was stirred at room temperature for 16 hours. To this was added 10 mM NaOAc solution (pH 4.5, 0.1 mL) and the reaction mixture was stirred at room temperature for another 24 hours. Crude was purified by prep-HPLC (Column: Phenomenex kinetex 5 μm C₁₈ 100 Å, 250×21.2 mm and using 5-50% ACN: 0.1% TFA in water as eluent, Flow rate: 21.2 mL/min, run time: 35 mins to afford the catabolites (40-90% yield) as white solid.

### LP-4-17

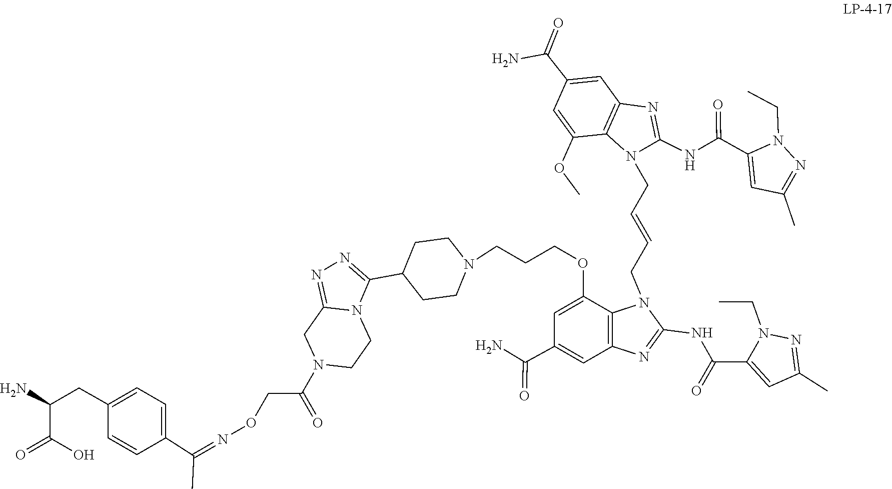 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(C[C@H](N)C(=O)O)cc4)C5)CC3)c21]

¹H NMR (400 MHz, DMSO): δ 12.89 (s, 2H), 8.26 (s, 2H), 8.01 (d, J=21.4 Hz, 2H), 7.71-7.56 (m, 3H), 7.44-7.25 (m, 5H), 6.55 (d, J=5.4 Hz, 2H), 5.75 (t, J=18.9 Hz, 2H), 5.12-4.75 (m, 7H), 4.53 (dt, J=14.6, 7.6 Hz, 4H), 4.21 (s, 1H), 3.99 (d, J=20.4 Hz, 6H), 3.72 (d, J=6.5 Hz, 4H), 3.11 (d, J=6.6 Hz, 4H), 2.92 (d, J=37.7 Hz, 3H), 2.26 (d, J=8.2 Hz, 3H), 2.12 (dd, J=9.5, 5.1 Hz, 8H), 1.91 (s, 4H), 1.28 (dq, J=11.0, 5.4 Hz, 6H).

### LP-4-18

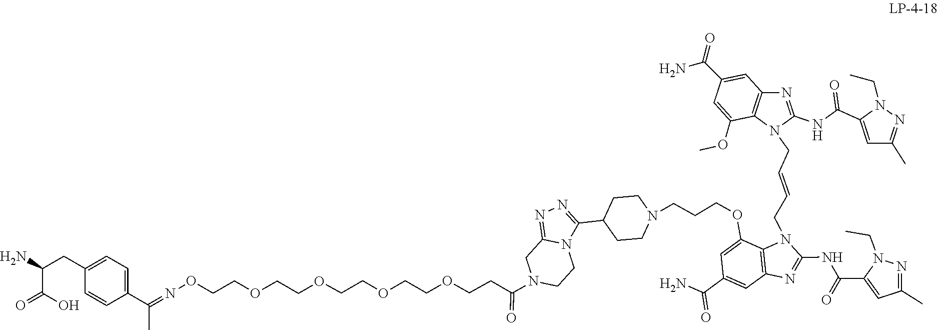 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCON=C(C)c4ccc(C[C@H](N)C(=O)O)cc4)C5)CC3)c21]

¹H NMR (400 MHz, DMSO): δ 12.89 (s, 2H), 8.25 (s, 3H), 8.00 (d, J=19.2 Hz, 2H), 7.65 (dd, J=18.4, 6.4 Hz, 3H), 7.37 (d, J=21.7 Hz, 3H), 7.30 (d, J=8.4 Hz, 3H), 6.54 (d, J=6.2 Hz, 2H), 5.78 (d, J=18.7 Hz, 2H), 4.92 (d, J=15.9 Hz, 4H), 4.78 (s, 1H), 4.62-4.46 (m, 4H), 4.27-4.18 (m, 3H), 3.98 (d, J=28.4 Hz, 6H), 3.79-3.62 (m, 7H), 3.58-3.48 (m, 31H), 3.11 (dd, J=6.6, 4.3 Hz, 4H), 2.22-2.09 (m, 7H), 1.91 (t, J=11.4 Hz, 3H), 1.28 (dt, J=16.4, 7.1 Hz, 6H).

### LP-4-19

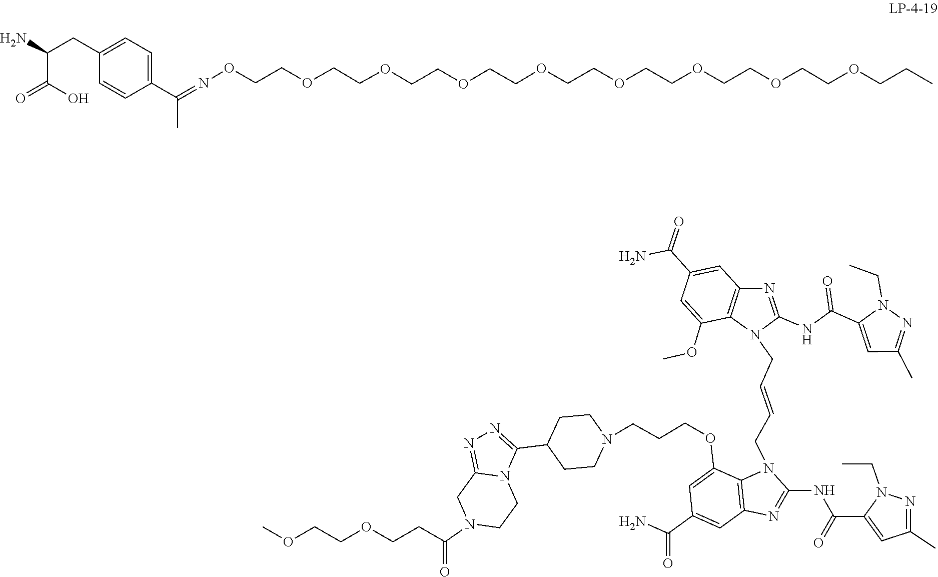

¹H NMR (400 MHz, DMSO): δ 12.89 (s, 2H), 8.25 (s, 3H), 8.00 (d, J=19.2 Hz, 2H), 7.65 (dd, J=18.4, 6.4 Hz, 3H), 7.37 (d, J=21.7 Hz, 3H), 7.30 (d, J=8.4 Hz, 3H), 6.54 (d, J=6.2 Hz, 2H), 5.78 (d, J=18.7 Hz, 2H), 4.92 (d, J=15.9 Hz, 4H), 4.78 (s, 1H), 4.62-4.46 (m, 4H), 4.27-4.18 (m, 3H), 3.98 (d, J=28.4 Hz, 6H), 3.79-3.62 (m, 7H), 3.58-3.48 (m, 31H), 3.11 (dd, J=6.6, 4.3 Hz, 4H), 2.22-2.09 (m, 7H), 1.91 (t, J=11.4 Hz, 3H), 1.28 (dt, J=16.4, 7.1 Hz, 6H).

### LP-4-20

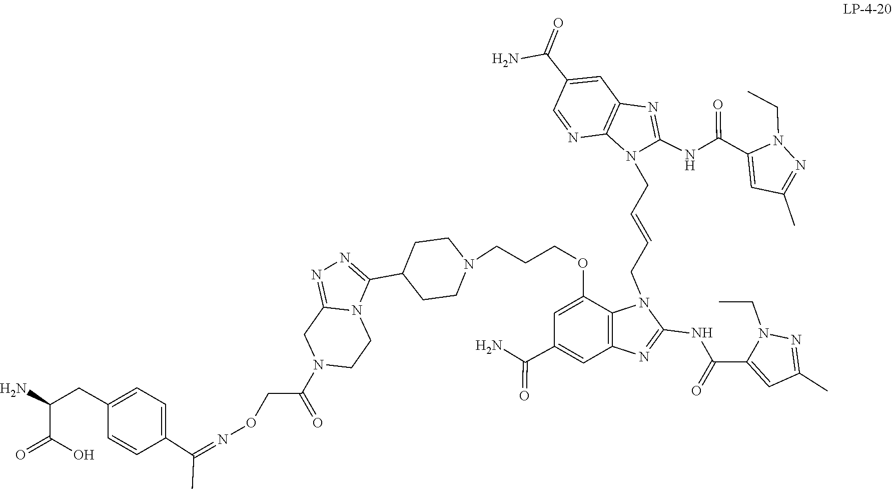 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(C[C@H](N)C(=O)O)cc4)C5)CC3)c21]

¹H NMR (400 MHz, DMSO): δ 12.90 (s, 2H), 8.72 (d, J=10.2 Hz, 1H), 8.32-8.22 (m, 2H), 8.15 (d, J=17.6 Hz, 2H), 7.97 (s, 1H), 7.67 (s, 1H), 7.63-7.55 (m, 2H), 7.42-7.26 (m, 4H), 6.51 (dd, J=14.0, 5.7 Hz, 2H), 5.95 (d, J=15.1 Hz, 1H), 5.76 (d, J=15.4 Hz, 1H), 5.08-4.86 (m, 5H), 4.79 (d, J=6.2 Hz, 3H), 4.60-4.44 (m, 4H), 4.26-4.02 (m, 6H), 3.95 (s, 7H), 3.11 (d, J=6.4 Hz, 2H), 2.99 (s, 3H), 2.26 (d, J=7.1 Hz, 3H), 2.10 (d, J=4.2 Hz, 12H), 1.27 (q, J=6.7 Hz, 6H)

### Synthesis of (S)-2-amino-3-(4-((E)-1-(((6-(((S)-1-(3-(1-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)amino)-6-oxohexyl)oxy)imino)ethyl)phenyl)propanoic acid (LP-57-3)

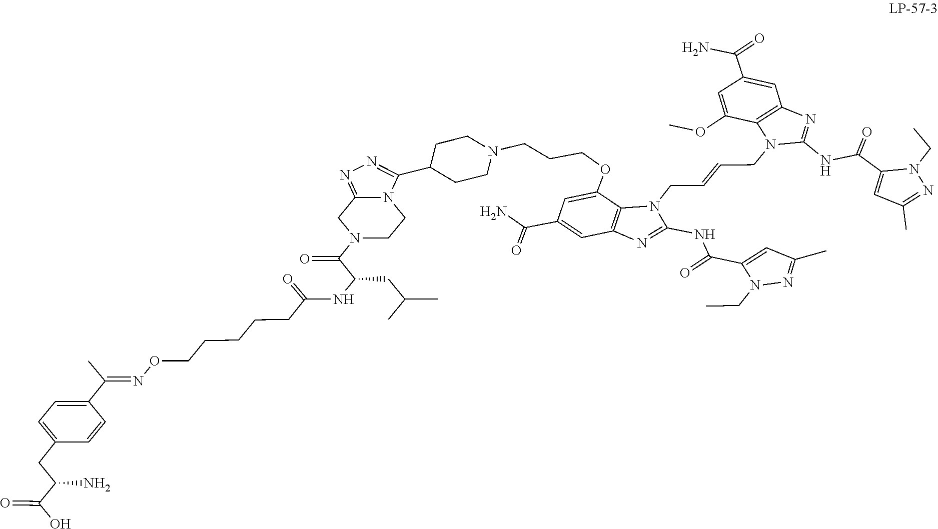 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)CCCCCO/N=C(\C)c4ccc(C[C@H](N)C(=O)O)cc4)C5)CC3)c21]

To (E)-7-(3-(4-(7-((6-(aminooxy)hexanoyl)-L-leucyl)-5,6,7,8-tetrahydro-[1,2,4]-triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide (LP-57-2, 15 mg, 0.012 mmol) and pAcF·HCl (15.0 mg, 0.062 mmol) in DMSO (2.0 mL) was added 10 mM sodium acetate buffer (0.5 mL). The reaction mixture was stirred at RT overnight. The reaction mixture was purified by reverse phase preparative HPLC (acetonitrile/water+0.1% TFA) to afford (S)-2-amino-3-(4-((E)-1-(((6-(((S)-1-(3-(1-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7 (8H)-yl)-4-methyl-1-oxopentan-2-yl)amino)-6-oxohexyl)oxy)imino)ethyl)phenyl)propanoic acid (LP-57-3) as its TFA salt. ¹H NMR (400 MHz, DMSO) δ 12.88 (s, 2H), 9.34 (s, 1H), 8.25 (s, 4H), 8.02 (s, 1H), 7.97 (s, 2H), 7.70-7.58 (m, 2H), 7.40 (s, 2H), 7.31 (dd, J=16.9, 8.5 Hz, 4H), 6.57-6.48 (m, 2H), 5.86-5.73 (m, 2H), 4.91 (s, 5H), 4.54 (dd, J=15.6, 7.6 Hz, 4H), 4.22 (s, 1H), 4.06 (s, 6H), 4.01 (s, 1H), 3.95 (s, 1H), 3.77-3.69 (m, 3H), 3.45 (t, J=6.0 Hz, 1H), 2.99 (s, 1H), 2.88 (s, 2H), 2.20-2.08 (m, 9H), 1.91 (s, 3H), 1.64 (s, 2H), 1.54 (s, 3H), 1.34-1.22 (m, 6H), 0.90 (d, J=6.3 Hz, 5H).

### Example 4. General Antibody Conjugation Using Select STING Agonist Linker Payloads

Select STING aminooxy linker payloads described herein were dissolved in DMSO to a final concentration of 50 mM. Oxime ligation reactions using a corresponding aminooxy linker payload to an FRαantibody, or aTROP-2 antibody were performed in 0.1 M sodium acetate, pH 4.5 buffer. All reactions were performed at antibody concentrations between 5-50 mg/mL and with a 2-50 molar fold excess of linker payload to moles of para-acetyl phenyl alanine (pAcF) non-natural amino acid labeled FRa, aTROP-2 antibody. A final amount of 3-35% DMSO was included to ensure solubility of the linker payload. All reactions were allowed to run at temperatures ranging between 22-37° C., for 4 to 96 hours. LCMS methods were used to measure DAR analysis.

TABLE 1
Conjugation efficiencies of various linker payloads after 18 hours
in 0.1M acetate.

| | | % | Drug to |
|---|---|---|---|
| | | Conjugation | Antibody ratio |
| Ab Conjugate | LP (compound) | efficiency | (DAR) |
| Conjugate 1-1 | LP-1-1 | 80 | 1.6 |
| Conjugate 1-2 | LP-1-2 | 98 | 1.9 |
| Conjugate 1-3 | LP1-3 | 96 | 1.8 |
| Conjugate 6-1 | LP-6-1 | 90 | 1.8 |
| Conjugate 2-2 | LP-2-2 | 90 | 1.8 |
| Conjugate 16-1 | LP-16-1 | 93 | 1.8 |
| Conjugate 2-1 | LP-2-1 | 95 | 1.9 |
| Conjugate 10-1 | LP-10-1 | 95 | 1.9 |

### Example 5. THP-1 Reporter Assays for Select STING Agonists

The activities of the STING agonists to activate human or mouse STING pathways were evaluated in THP-1-Dual™, THP1-Dual™ KI-hSTING-R232, THP1-Dual™ KI-hSTING-H232 Cells and THP1-Dual™ KI-mSTING Cells. THP-1-Dual™ cells contains hSTING-HAQ haplotype and can be used to evaluate the activity of STING agonists by monitoring the activation of NF-κB pathway and the TRF (IFN regulatory factor) pathways. THP1-Dual™ KO-STING cells were generated from THP1-Dual™ cells by stable knockout of the endogenous HAQ STING gene. THP1-Dual™ KI-hSTING-R232 cells, THP1-Dual™ KI-hSTING-H232 cells and THP1-Dual™ KI-mSTING cells were generated by transfecting THP1-Dual™ KO-STING cells with hSTING-R232 or hSTING-H232 or mSTING. Stimulating these reporter cells with a STING agonist activates TRF pathway and induces the production of a secreted Lucia luciferase in the cell culture medium, which can be determined using QUANTI-Luc™ reagent.

THP-1-Dual™, THP1-Dual™ KI-hSTING-R232, THP1-Dual™ KI-hSTING-H232 and THP1-Dual™ KI-mSTING reporter cells were purchased from Invivogen and maintained in manufacture recommended culture medium with required supplemental antibiotics. On the day of assay, the cells were harvested with Accutase and counted by the Vi-CELL Cell Viability Analyzers. Cells were resuspended in HEK blue detection medium and a total of 10000 cells were seeded in each well of a 384-well flat bottom plate. Serial dilutions of STING agonists free drugs (1:3 serial dilution starting from 5 uM) was added into treatment wells. After incubation at 37° C. in a CO₂ incubator for 24 hours, 20 μl of cell culture medium per well was transferred into a 384-well white (opaque) plate. 50 μl of QUANTI-Luc™ assay solution was then added to each well and the plate was immediately transferred to an ENVISION® plate reader to measure luminescence (Perkin-Elmer). Relative luminescence readings were converted to fold change of signal using untreated cells as controls. Data was fitted with non-linear regression analysis, using log (agonist) vs. response, variable slope, 4-parameter fit equation using GraphPad Prism. Data was expressed as fold change in signal vs. dose of STING agonist in nM with error bars indicating the Standard Deviation (SD) of the quadruplicates. There result was reported in Table 2 as EC₅₀ (the midpoint of the curve, or concentration at which 5000 of the maximum effect was observed).

Table 2 provides the results for THP-1 reporter induction human/mouse reporter cells. (“A” means EC₅₀≤50 nM; “B” means >50 nM and <150 nM; “C” means >150 nM). Reference Compd A and Reference Compd B are below:

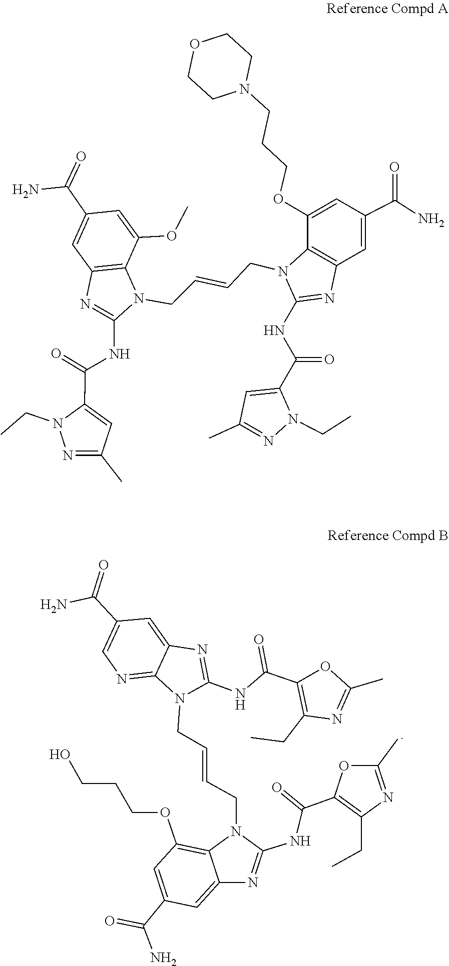 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCOCC3)c21 ; CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCO)c21]

TABLE 2
THP-1 reporter assay EC₅₀

| | Reporter assay EC₅₀ (nM) | | | | |
|---|---|---|---|---|---|
| | THP-1- | THP-1- | THP-1 | THP-1 | 293- |
| Compd No. | R232 | H232 | HAQ | mSTING | mSTING |
| Reference Compd A | A | A | A | | A |
| Reference Compd B | A | A | C | A | |
| 6 | A | A | A | | B |
| 2 | A | A | B | | C |
| 3 | B | B | B | | C |
| 1 | A | A | A | | C |
| LP-1-1 | C | C | B | | C |
| LP-1-4 | C | C | C | | C |
| 63 | C | C | C | C | |
| LP-6-1 | A | A | A | A | |
| 16 | A | A | A | A | |
| 17 | A | A | A | A | |
| 23 | A | A | B | B | |
| 21 | B | B | C | C | |
| 22 | C | C | C | C | |
| 26 | A | A | C | B | |
| 25 | A | A | C | A | |
| 24 | A | B | C | C | |
| 4 | B | C | B | C | |
| 5 | C | C | C | C | |
| 18 | B | C | B | C | |
| 19 | A | B | B | A | |
| 20 | A | B | B | A | |
| 7 | C | C | C | C | |
| 8 | C | C | C | C | |
| 62 | C | C | C | C | |
| 9 | C | C | C | C | |
| LP-2-3 | B | B | C | C | |
| 11 | A | A | C | A | |
| 10 | A | A | A | A | |
| 12 | C | C | C | C | |
| 13 | A | A | A | A | |
| 14 | A | A | A | A | |
| 15 | C | C | C | C | |
| 43 | C | C | C | C | |
| 44 | A | A | A | A | |

Surface plasmon resonance assay experimental methods were followed based on Pan et al., 2020, Science 369, 935 (“A” means EC₅₀≤20 nM); (“B” means EC₅₀≥20 nM). The results are shown in Table 3.

TABLE 3
Surface plasmon resonance

| Compound | hSTING WT | hSTING H232 | hSTING AQ |
|---|---|---|---|
| ID | SPR (Kd) nM | SPR (Kd) nM | SPR (Kd) nM |
| Reference | A | A | A |
| Compd A | | | |
| Reference | A | A | A |
| Compd B | | | |
| 6 | A | A | A |
| 2 | A | A | A |
| 3 | A | A | A |
| 1 | A | A | A |
| LP-1-1 | A | A | A |
| LP-1-4 | A | A | A |
| LP-6-1 | A | A | A |
| 17 | A | A | A |
| 21 | A | A | A |
| 22 | A | A | A |
| 4 | A | A | A |
| 5 | B | B | B |
| 8 | B | B | B |
| LP-2-3 | A | A | A |

### Example 6: In Vitro Human Cancer Cell and PBMCs Co-Culture Assays

The ability of different TROP2 ADC and iSACs to induce cancer cell killing, immune activation, and cytokine release was evaluated in an in vitro co-culture assay with human cancer cells and human PBMCs.

Human PBMCs were isolated from healthy human blood donors (StemCells) by Leukosep tube (Greiner Bio-One) and Nycoprep 1.077 buffer (Progen) according to manufacturer's instructions. Residual RBCs were removed using ACK lysis buffer (Gibco) for 10 minutes, followed by DPBS wash. Isolated PBMCs were counted, resuspended in freezing medium (Sigma) and stored in a freezing vessel at −80 C for 24 hours before being transferred to vapor-phase liquid nitrogen for long-term storage.

For the co-culture assay, cancer cells were plated on transparent flat bottom polystyrene plates and allowed to attach overnight. On the next day, serially diluted (1:4) test articles were formulated at 3× starting concentration in complete RPMI media and added onto each well. Human PBMCs were thawed and resuspended in RPMI supplemented with 10% heat-inactivated fetal bovine serum from Hyclone, 1% Penicillin/Streptomycin and 2 mmol/L-glutamax. PBMCs were then added into the same well at a final ratio of PBMCs:cancer cells =30:1. Plates were incubated for 2 or 3 days at 37° C. with 5% CO2 in a cell culture incubator.

On the day of sample collection, cell culture supernatants were collected and stored at −80° C. until cytokine release analyses were performed. All the cells in the well were combined (both suspension and adherent cells) and resuspended in 50 ul of PBS containing Live/Dead Fixable Violet Dead Cell Stain (Thermo Fisher) and 1× human Fc block (BD Biosciences) and incubated at 37° C. for 20 minutes. After washing with complete RPMI-1640 media and FACS buffer (DPBS buffer with 1% bovine serum albumin and 0.05% v/v sodium azide), cells were resuspended in 25 ul per well of a primary antibody concoction in FACS binding buffer containing 1:20 dilution of BD Horizon Brilliant Stain Buffer Plus, live/Dead staining and a series of antibodies for gating different immune cells and activation markers. After incubation on ice for 1 hour, cells were washed with FACS buffer and fixed with 50 ul/well of 2% PFA (paraformaldehyde) for 20 minutes. Cells were then washed with FACS buffer again and read on Cytek flow cytometer. FACS data analysis was done by Flowjo software to identifymonocytes, B cells, CD4+ T cells, CD8+ T cells, NK cells and Dendritic cells and their activation status. Data from Flowjo was exported and plotted using non-linear regression analysis, log (agonist/inhibitor) vs. response, variable slope, 4-parameter fit equation in GraphPad Prism.

To measure the cytokine released in this assay, customized MSD V-plex plates were ordered to measure human IL-6 and IP-10. The supernatant was thawed and diluted1:50 using dilution buffer provided by the vendor before added into the MSD plates. Cytokine measurement in the cell culture supernatant was carried out using protocols provided by the vendor. Raw data generated by the Meso Scale Discovery (MSD®) SQ120 plate reader were imported into MSD Discovery Workbench software. Unknown sample values were interpolated from standard curves by the software and imported into PRISM Graphpad. Each cytokine level was fitted with non-linear regression analysis, using log (agonist) vs. response, variable slope, 4-parameter fit equation.

### Example 7. TROP2 iSACs Conjugated to diAbzi STING Agonists with Different Potency and Different Linkers in Co-Culture Assays

The ability of different TROP2 iSACs to induce cancer cell killing, immune activation, and cytokine release was evaluated in an in vitro co-culture assay with TROP2 positive CaoV3 cells and human PBMCs. Anti-TROP2 h1925-C06 was conjugated to different STING agonist linker payloads at light chain K42 site.

As shown in Table 4 and Table 5, TROP2 ADC 4 induced potent target cell killing. All the iSACs tested in this experiment induced potent target cell killing with similar or better potency than the ADC control ADC4. All the iSACs tested also induced potent decrease of CD14+ cells (shown as % of CD14+ in total PBMCs), which is correlated with monocyte activation, as well as great increase of DCs (shown as % of CD11c+ in CD14− PBMCs) and DC activation. This data suggested that the iSACs induced monocyte activation and differentiation into DCs in this assay. iSACs also induced activation of other immune cells (B cells, CD4+ T cells, CD8+ T cells as well as NK cells) with similar trends (Table 5 and Table 6). The PEGylated bGlu cleavable DAR4 ADC conjugate was used as control.

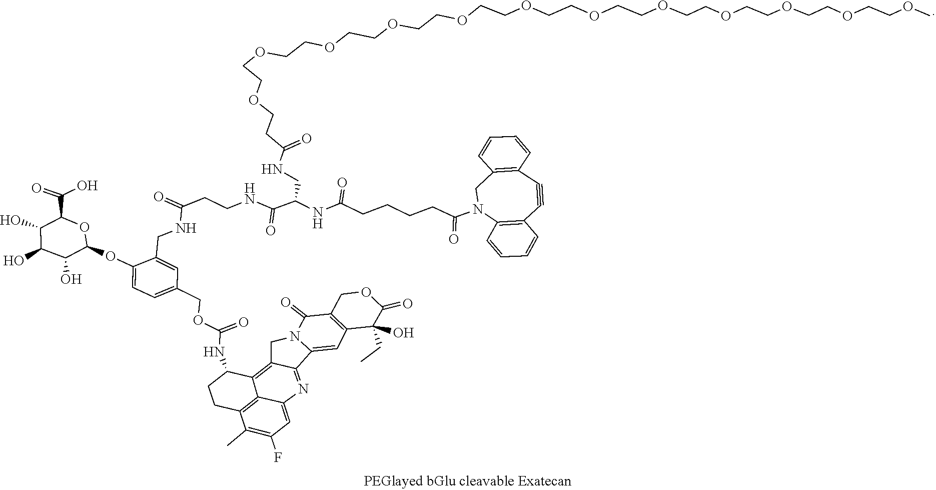

Activated immune cells release several cytokines to further promote immune cell recruitment and proliferation. While the TROP2 ADC induced only weak release of IP-10 and IL-6 in the co-culture system, TROP2 iSACs induced potent cytokine release (both IP-10 and IL-6) in the human tumor cells and PBMCs co-culture assay with trends similar to immune cell activation (Table 7).

In general, TROP2 iSAC10 conjugated to LP-10-1 induced slightly more potent immune cell activation than iSACs conjugated to LP-1-1, LP-1-3 or LP-2-1. iSAC9 conjugated to LP-2-1 (Compound 2 with a non-cleavable linker), showed slightly weaker activity compared to the others. iSAC also induced monocyte cell count decrease and immune cell activation similar to their corresponding ADCs.

TABLE 4
TROP2 iSACs induced tumor cell killing and immune cell
differentiation

| | | CAOV3/PBMC co-culture | | | | | |
|---|---|---|---|---|---|---|---|
| | | Target cell | | Monocyte | | | |
| ADC | | count | | count | | DC count | |
| or | | EC50 | Span | EC50 | Span | EC50 | Span |
| iSAC | Description | (nM) | (cell count) | (nM) | (%) | (nM) | (%) |
| ADC | h1925-C06 | 0.434 | 4100 | NC | NC | NC | NC |
| 4 | Y180/F404- | | | | | | |
| | PEGylated | | | | | | |
| | bGlu cleavable | | | | | | |
| | Exatecan/K42 | | | | | | |
| iSAC | h1925-C06 | 0.267 | 4374 | 0.24 | 17.7 | 0.32 | 9.53 |
| 1 | Y180/F404/ | | | | | | |
| | K42-LP-1-1 | | | | | | |
| iSAC | h1925-C06 | 0.115 | 5963 | 0.27 | 19.5 | 1.03 | 15.47 |
| 4 | Y180/F404/ | | | | | | |
| | K42-LP-1-3 | | | | | | |
| iSAC | h1925-C06 | 0.187 | 5539 | 0.35 | 20.5 | 1.03 | 12.12 |
| 9 | Y180/F404/ | | | | | | |
| | K42-LP-2-1 | | | | | | |
| iSAC | h1925-C06 | 0.072 | 4791 | 0.11 | 20.9 | 0.17 | 14.66 |
| 10 | Y180/F404/ | | | | | | |
| | K42-LP-10-1 | | | | | | |

NC = Not Calculable due to incomplete curve

TABLE 5
TROP2 iSACs induced immune cell activation

| | | CAOV3/PBMC co-culture | | | | | |
|---|---|---|---|---|---|---|---|
| | | Monocyte | | DC | | B cell | |
| ADC | | activation | | activation | | activation | |
| or | | EC50 | Span | EC50 | Span | EC50 | Span |
| iSAC | Description | (nM) | (%) | (nM) | (%) | (nM) | (%) |
| ADC | h1925-C06 Y180/ | NC | NC | 0.034 | 2.6 | 0.060 | 1.6 |
| 4 | F404-PEGylated | | | | | | |
| | bGlu cleavable | | | | | | |
| | Exatecan/K42 | | | | | | |
| iSAC | h1925-C06 Y180/ | 0.269 | 66.9 | 0.214 | 14.7 | 0.869 | 28.5 |
| 1 | F404/K42-LP-1-1 | | | | | | |
| iSAC | h1925-C06 Y180/ | 0.284 | 72.5 | 0.237 | 16.9 | 3.050 | 46.1 |
| 4 | F404/K42-LP-1-3 | | | | | | |
| iSAC | h1925-C06 Y180/ | 0.363 | 65.5 | 0.342 | 16.6 | 16.30 | 56.0 |
| 9 | F404/K42-LP-S-1 | | | | | | |
| iSAC | h1925-C06 Y180/ | 0.083 | 63.9 | 0.074 | 12.8 | 0.629 | 28.0 |
| 10 | F404/K42-LP-10-1 | | | | | | |

NC = Not Calculable due to incomplete curve

TABLE 6
TROP2 iSACs induced immune cell activation

| | | CAOV3/PBMC co-culture | | | | | |
|---|---|---|---|---|---|---|---|
| | | CD4+ T cell | | CD8+ T cell | | NK cell | |
| ADC | | activation | | activation | | activation | |
| or | | EC50 | Span | EC50 | Span | EC50 | Span |
| iSAC | Description | (nM) | (%) | (nM) | (%) | (nM) | (%) |
| ADC | h1925-C06 Y180/ | 0.017 | 0.55 | NC | NC | 0.301 | 0.8 |
| 4 | F404-(PEGylated | | | | | | |
| | bGlu cleavable | | | | | | |
| | Exatecan)/K42 | | | | | | |
| iSAC | h1925-C06 Y180/ | 0.149 | 6.70 | 0.386 | 4.0 | 0.360 | 4.4 |
| 1 | F404/K42-LP-1-1 | | | | | | |
| iSAC | h1925-C06 Y180/ | 0.236 | 6.71 | 0.135 | 3.9 | 0.155 | 5.3 |
| 4 | F404/K42-LP-1-3 | | | | | | |
| iSAC | h1925-C06 Y180/ | 0.149 | 8.59 | 0.104 | 4.1 | 0.282 | 6.0 |
| 9 | F404/K42-LP-2-1 | | | | | | |
| iSAC | h1925-C06 Y180/ | 0.041 | 6.13 | 0.034 | 3.5 | 0.061 | 6.5 |
| 10 | F404/K42-LP-10-1 | | | | | | |

NC = Not Calculable due to incomplete curve

TABLE 7
TROP2 iSACs induced cytokine release

| | | CAOV3/PBMC co-culture | | | |
|---|---|---|---|---|---|
| ADC | | IP-10 | | IL-6 | |
| or | | EC50 | Span | EC50 | Span |
| iSAC | Description | (nM) | (ng/ml) | (nM) | (ng/mL) |
| ADC | h1925-C06 Y180/F404- | 23.8 | 1030 | NC | NC |
| 4 | PEGylated bGlu | | | | |
| | cleavable Exatecan /K42 | | | | |
| iSAC | h1925-C06 Y180/F404/ | NT | NT | NT | NT |
| 1 | K42-LP-1-1 | | | | |
| iSAC | h1925-C06 Y180F404// | 1.31 | 460 | 1.02 | 1207 |
| 4 | K42-LP-1-3 | | | | |
| iSAC | h1925-C06 Y180/F404/ | NC | NC | 0.45 | 84 |
| 9 | K42-LP-2-1 | | | | |
| iSAC | h1925-C06 Y180/F404/ | 0.208 | 939 | 0.12 | 1095 |
| 10 | K42-LP-10-1 | | | | |

NC = Not Calculable due to incomplete curve,
NT = Not Tested

To further investigate the linker property and potency of the TROP2 iSACs conjugated to STING agonist, Compound S-3 with different length linkers were further evaluated in another vitro co-culture assay with TROP2 positive CaoV3 cells and human PBMCs.

In this assay (Table 8), TROP2 iSAC13, iSAC16 and iSAC20 induced some cell killing that is weaker than or similar to the iSAC1, while iSAC11, iSAC12 and iSAC14 did not induce significant cell killing. TROP2 iSAC13, iSAC16, iSAC20 and iSAC 14 also induced decrease of monocytes, while iSAC11 and iSAC12 did not. iSAC11 and iSAC12 induced weak activation of monocytes while the other iSAC induced potent activation of monocytes. Similar trend observed for these iSAC on DC, B cell, T cell and NIK cell activation (data not shown).

TABLE 8
TROP2 iSACs induced tumor cell killing and immune cell activation

| | | CAOV3/PBMC co-culture | | | | | |
|---|---|---|---|---|---|---|---|
| | | | | Monocyte | | Monocyte | |
| | | Cancer Cell Count | | Cell Count | | Activation | |
| | | IC₅₀ | Span | IC₅₀ | Span | EC₅₀ | Span |
| iSAC | Description | (nM) | (Cell Count) | (nM) | (%) | (nM) | (%) |
| iSAC | h1925-C06 | 0.14 | 14,926 | 0.21 | 21.7 | 0.007 | 24 |
| 1 | Y180/F404/ | | | | | | |
| | K42-LP-1-1 | | | | | | |
| iSAC | h1925-C06 | NC | <5000 | NC | <5 | 1.207 | 19 |
| 11 | Y180/F404/ | | | | | | |
| | K42-LP-21-1 | | | | | | |
| iSAC | h1925-C06 | NC | <5000 | NC | <5 | 2.714 | 16 |
| 12 | Y180/F404/ | | | | | | |
| | K42-LP-4-1 | | | | | | |
| iSAC | h1925-C06 | 0.74 | 13257 | 0.46 | 13.7 | 0.097 | 20 |
| 13 | Y180/F404/ | | | | | | |
| | K42-LP-4-2 | | | | | | |
| iSAC | h1925-C06 | NC | <5000 | 26.22 | 17.9 | 0.094 | 10 |
| 14 | Y180/F404/ | | | | | | |
| | K42-LP-4-3 | | | | | | |
| iSAC | h1925-C06 | 1.05 | 15,566 | 1.64 | 15.6 | 0.922 | 27 |
| 16 | Y180/F404/ | | | | | | |
| | K42-LP-4-5 | | | | | | |
| iSAC | h1925-C06 | 0.100 | 16,114 | 0.28 | 15.7 | 0.074 | 27 |
| 20 | Y180/F404/ | | | | | | |
| | K42-LP-4-4 | | | | | | |

NC = Not Calculable due to incomplete curve

### Example 7. TROP2 iSACs in Co-Culture Assay with PBMCs and TROP2 Positive or Negative Tumor Cells

TROP2 iSACs conjugated to diABZI STING agonists with different potency and linkers were also tested in vitro co-culture assay with TROP2 positive NCI-1H292 cells or TROP2 negative NCI-1H460 cells and human PBMCs. Exatecan —was added as a control. The EC50 and span for target tumor cell killing, monocyte % in the PBMCs, monocyte activation and IP-10 release were summarized in Table 9 and Table 10. Similar trend in other immune cell activation were observed (CD4+T, CD8+ T cells, NK, and B cell) and cytokine release (IFNγ, TNFα, IL-6, IL-1α and MIP-1α) but not shown.

As expected, Exatecan induced potent tumor cell killing with a similar IC50 on both TROP2 positive and negative cells and did not induce activation of any immune populations (Table 11 and Table 15). TROP2 ADC SP 11817 induced potent cell killing on TROP2 positive cells but not on TROP2 negative cells and did not induce much activation of any immune populations (Table 16). TROP2 iSACs did not induce any tumor cell killing in either TROP2 positive or negative co-cultures.

When PBMCs were co-cultured with TROP2 positive NCI-H292 cells, iSAC1 and iSAC2 conjugated to the same STING linker payload LP-1-1 Compound 1 with non-cleavable linker), showed the same immune cell activity and cytokine release. iSAC3 conjugated to LP-4-3 (Compound 4 with non-cleavable linker), induced much less immune cell activation and cytokine release, which is correlated to the lower STING activity for Compound 4 compared to Compound 1 (Table 9).

When PBMCs were co-cultured with TROP2 negative NCI-H460 cells (Table 10), iSAC1 and iSAC2 induced some activation of immune cells, while iSAC3 did not induce any immune cells activation or cytokine release.

TABLE 9
TROP2 ADC, iSACs induced tumor cell killing and immune cell
activation

| | | NCI-H441/PBMC co-culture | | | | | | | |
|---|---|---|---|---|---|---|---|---|---|
| | | Cancer Cell | | Monocyte Cell | | Monocyte | | | |
| ADC | | Count | | Count | | Activation | | IP-10 release | |
| and | | IC₅₀ | Span | IC₅₀ | Span | EC₅₀ | Span | EC₅₀ | Span |
| iSAC | Description | (nM) | (Cell Count) | (nM) | (%) | (nM) | (%) | (nM) | (pg/mL) |
| | Exatecan free drug | 0.44 | 5,749 | NC | NC | NC | NC | NO | NC |
| ADC | h1925-C06 | 0.37 | 2,784 | NC | NC | NC | NC | NO | NC |
| 4 | Y180/F404- | | | | | | | | |
| | PEGylated bGlu | | | | | | | | |
| | cleavable | | | | | | | | |
| | Exatecan/K42 | | | | | | | | |
| ADC6 | h1925-C06 | NC | NC | NC | NC | NC | NC | NC | NC |
| | F241/F404- | | | | | | | | |
| | PEGylated bGlu | | | | | | | | |
| | cleavable | | | | | | | | |
| | Exatecan/K42 | | | | | | | | |
| iSAC | h1925-C06 | NO | NC | 0.05 | 20 | 0.09 | 6.8 | 0.14 | 36,451 |
| 1 | Y180/F404/ | | | | | | | | |
| | K42-LP-1-1 | | | | | | | | |
| iSAC | h1925-C06 | NC | NC | 0.05 | 20 | 0.07 | 6.4 | 0.19 | 32,480 |
| 2 | Y180/F404/ | | | | | | | | |
| | K42-LP-2-1 | | | | | | | | |
| iSAC | h1925-C06 | NC | NC | 0.15 | 13 | 2.9 | 2.4 | 9.12 | 14,062 |
| 14 | Y180/F404/ | | | | | | | | |
| | K42-LP-4-3 | | | | | | | | |

TABLE 10
TROP2 ADC, iSACs induced tumor cell killing, immune cell
activation and cytokine release

| | | NCI-H460/PBMC co-culture | | | | | | | |
|---|---|---|---|---|---|---|---|---|---|
| | | Cancer Cell | | Monocyte Cell | | Monocyte | | | |
| ADC | | Count | | Count | | Activation | | IP-10 release | |
| and | | IC50 | Span | IC₅₀ | Span | EC₅₀ | Span | EC₅₀ | Span |
| iSAC | Description | (nM) | (Cell Count) | (nM) | (%) | (nM) | (%) | (nM) | (pg/mL) |
| | Exatecan | 0.38 | 23,530 | NC | NC | NC | NC | NC | NC |
| ADC | h1925-C06 | NC | NC | NC | NC | NC | NC | NC | NC |
| 4 | Y180/F404 | | | | | | | | |
| | PEGylated bGlu | | | | | | | | |
| | cleavable | | | | | | | | |
| | Exatecan/K42 | | | | | | | | |
| iSAC | h1925-C06 | NC | NC | NC | NC | 0.69 | 1.70 | NC | NC |
| 1 | Y180/F404/ | | | | | | | | |
| | K42-LP-1-1 | | | | | | | | |
| iSAC | h1925-C06 | NC | NC | NC | NC | 0.40 | 1.40 | NC | NC |
| 2 | Y180/F404/ | | | | | | | | |
| | K42-LP-2-1 | | | | | | | | |
| iSAC | h1925-C06 | NC | NC | NC | NC | NC | NC | NC | NC |
| 14 | Y180/F404/ | | | | | | | | |
| | K42-LP-4-3 | | | | | | | | |

NC = Not Calculable due to incomplete curve

### Example 8. iSACs Induced Cytokine Release in Cancer Cells

To evaluate if TROP2 iSACs will result in cytokine release in TROP2 positive cells, NCI-H292 was treated with test articles for 120 hours. The cell supernatant was collected and IP-10 and IL-6 released in the cell culture medium was measured using Streptavidin single spot 96 well plates, compatible human antibody pairs, and calibrating solutions commercially available from MesoScale Discovery. Raw data generated by the Meso Scale Discovery (MSD®) SQ120 plate reader was imported into MSD Discovery Workbench software. Unknown sample values were interpolated from standard curves by the software and imported into Graphpad Prism. Each cytokine level was fitted with non-linear regression analysis, using log (agonist) vs. response, variable slope, 4-parameter fit equation in GraphPad Prism.

As shown in Table 11, STING agonists Compound S-1 and Compound S-3, but not exatecan, were able to induce IP-10 and IL-6 release from NCI-H292 cells at EC₅₀ values greater than 100 nM. TROP2 ADC 4 did not induce IP-10 or IL-6 above the baseline of untreated cells. iSAC 1, iSAC 2, iSAC 14, resulted in similar overall induction of IP-10 (EC₅₀˜2 nM; Span ˜2000). In contrast to these findings, the magnitude of IL-6 induction by iSAC 1, iSAC 2, and iSAC 14 (EC₅₀ 2-4 nM; Span ˜6000). These results indicate that release of IP-10 and IL-6 are primarily dictated by STING agonist activity for iSACs.

TABLE 11
TROP2 iSACs induced cytokine release on TROP2
positive NCI-H292 Cells

| | | IP-10 Release | | IL-6 Release | |
|---|---|---|---|---|---|
| ADC or | | EC₅₀ | Span | EC₅₀ | Span |
| iSAC | Description | (nM) | (pg/mL) | (nM) | (pg/mL) |
| | Exatecan | 0.87 | 203 | 14.69 | 1410 |
| Compound | STING agonist | >100 | >4000 | >100 | >10000 |
| 1 | | | | | |
| Compound | STING agonist | >100 | >4000 | >100 | >8000 |
| 4 | Compound 4 | | | | |
| iSAC | h1925-C06 Y180/F404/ | 2.91 | 2351 | 3.8 | 6987 |
| 1 | K42-LP-1-1 | | | | |
| iSAC | h1925-C06 Y180/F404/ | 1.54 | 2067 | 3.62 | 6011 |
| 2 | K42-LP-2-1 | | | | |
| iSAC | h1925-C06 Y180/F404/ | 1.59 | 1996 | 1.74 | 6790 |
| 14 | K42-LP-4-3 | | | | |

NC = Not Calculable due to incomplete curve

### Example 9. TROP2 ADCs and iSACs Induced Immunogenic Cell Death

Immunogenic cell death elicited by the TROP2 ADC, iSAC, and exatecan were measured by cell surface translocate of calreticulin and release of HMGB1 in the cell culture supernatant. Briefly, TROP2 positive cell line NCI-H292 were seeded into a 96-well flat bottom plate 4 hours prior to the initialization of the assay. Test articles were formulated at a 2× starting concentration in cell culture medium, and samples were serial diluted (1:3) under sterile conditions and added onto the cells in duplicates. After incubation at 37° C. in a CO₂ incubator for 48 hours, the cell supernatant was collected for measuring HMGB1 and the adherent cells were collected for calreticulin expression on the cell surface.

HMGB1 released in the supernatant was assessed via sandwich ELISA using mouse anti-human HMGB1 clone 2F6 (Sigma-Aldrich) as the capture, polyclonal rabbit anti-human HMGB1 (Abcam) as secondary, and polyclonal goat anti-rabbit IgG HRP as the detection antibody. The HMGB1 concentrations in the cell culture medium were calculated based a standard curve built on commercially available recombinant human HMGB1 (R&D Systems).

NCI-H292 cells were detached using Accutase (Innovative Cell Technologies) and viability was accessed using a 1:500 dilution of Zombie NIR viability dye. Calreticulin induction was measured via commercial anti-calreticulin antibody conjugated to Alexa Fluor 647 (Abcam) diluted at 1:60. Surface calreticulin measures on the live cells were gathered using an Attune NXT Flow Cytometer (ThermoFisher Scientific) to obtain median fluorescence intensity values. All data were fitted with non-linear regression analysis, using log(agonist) vs. response, variable slope, 4 parameter fit equation using GraphPad Prism.

As shown in Table 12, exatecan, ADC 4 and Free STING agonists Compound S-1 and Compound S-3, and the corresponding iSACs (iSAC 1, iSAC 2, and iSAC 14) resulted in reduced release of HMGB1 (˜30 ng/mL) and no induction of CALR when compared to untreated control cells. These data indicate that immunogenic cell death is primarily driven by exatecan and not STING agonist Compound S-1 or Compound S-3, although the STING agonists may be sufficient to induce cell stress given HMGB1 release and lack of CALR induction.

TABLE 12
TROP2 iSACs induced immunogenic cell death markers on
NCI-H292 cells

| | | HMGB-1 Release | | Calreticulin | |
|---|---|---|---|---|---|
| Cpd | | EC₅₀ | Span | EC₅₀ | Span |
| number | Description | (nM) | (ng/mL) | (nM) | (MFI) |
| | Exatecan | 2.08 | 55 | 0.19 | 783 |
| Compd | STING agonist | 6.06 | 17 | NC | NC |
| 1 | | | | | |
| Compd | STING agonist (4) | 7.45 | 11 | NC | NC |
| 4 | | | | | |
| ADC | h1925-C06 Y180/F404- | 2.38 | 47 | 0.53 | 1121 |
| 4 | PEGylated bGlu | | | | |
| | cleavable Exatecan/K42 | | | | |
| iSAC | h1925-C06 Y180/F404/ | 1.73 | 16 | NC | NC |
| 1 | K42-LP-1-1 | | | | |
| iSAC | h1925-C06 Y180/F404/ | 2.71 | 9 | NC | NC |
| 2 | K42-LP-2-1 | | | | |
| iSAC | h1925-C06 Y180/F404/ | 1.29 | 16 | NC | NC |
| 14 | K42-LP-4-3 | | | | |

NC = Not Calculable due to incomplete curve

### Example 10. Anti-Tumor Activity of aTROP2 iSACs in a Syngeneic Mouse Tumor Model

C57BL/6 mice were implanted with 0.2×10⁶ MC38-hTrop2 tumor cells subcutaneously. Mice were randomized and enrolled into the study 8 days post implant, with tumor sizes around 100-115 mm³. Tumor-bearing mice were administered a single dose of the test articles at doses ranging from 0.5 mg/kg to 1 mg/kg. All treatments were well tolerated with normal body weight gain throughout the course of the study.

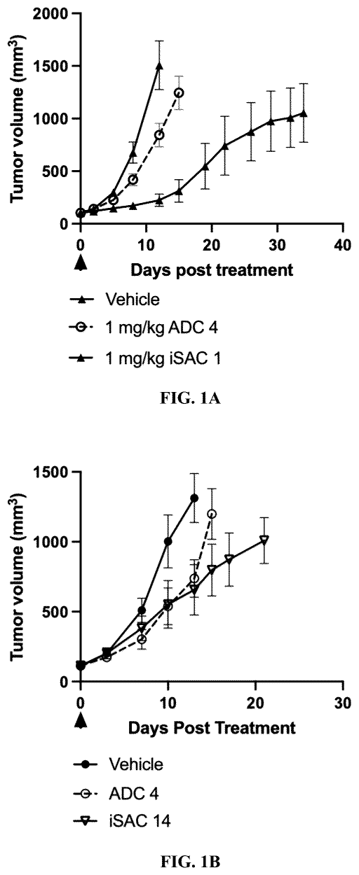

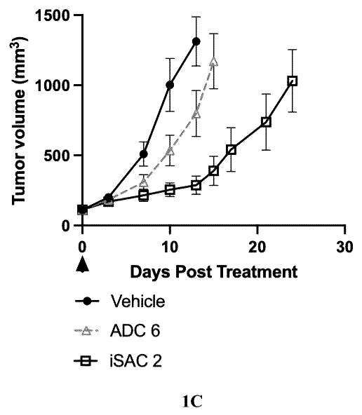

FIG. 1A-FIG. 1C summarize effects of TROP2 conjugates with and without STING agonist on growth of MC38-hTrop2 tumors. Analysis of treatment groups was done on day 12, when the mean of vehicle-treated tumors reached the study endpoint (>1,500 mm³). At a dose of 1 mg/kg, ADC 4 and ADC 6 induced comparable anti-tumor activity (48% and 43% TGI respectively). In comparison, iSAC 1 induced potent anti-tumor activity at both 0.5 mg/kg and 1 mg/kg (83% and 92% TGI respectively) and greater anti-tumor activity compared to 1 mg/kg ADC 4 (FIG. 1A). At both 0.5 mg/kg and 1 mg/kg, treatment with TROP2 iSAC 1 also resulted in 10-20% tumor free mice. At a 1 mg/kg dose treatment with ADC 4 and iSAC 14 induced comparable anti-tumor activity (48% and 55% TGI respectively) (FIG. 1B). At 1 mg/kg dose, treatment with iSAC 2 induced greater anti-tumor activity compared to ADC 6 (86% TGI and 43% TGI respectively) (FIG. 1C).

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

What is claimed is:

1. A compound of Formula (I) or (IV): 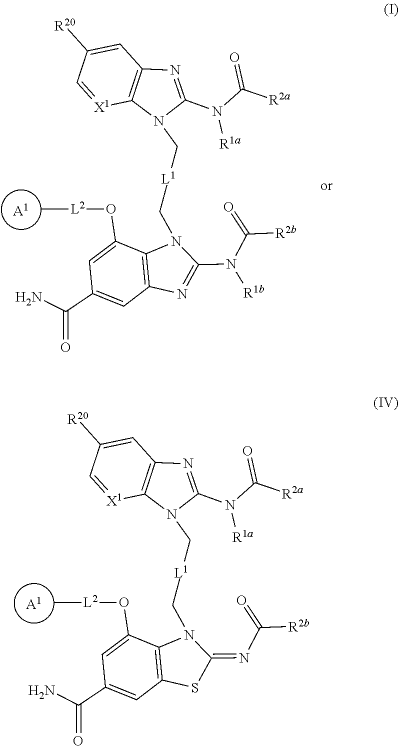
or a pharmaceutically acceptable salt or tautomer thereof,
wherein
X¹ is selected from N and CR³;
R²⁰ is selected from hydrogen and —CON(R^{3a})(R^{3b});
R^{1a}, R^{1b}, R^{3a} and R^{3b} are independently selected from hydrogen and optionally substituted C₁₋₆ alkyl, wherein the C₁₋₆ alkyl is optionally substituted with one or more R⁵⁰;
R^{2a} and R^{2b} are independently selected from:
optionally substituted C₁₋₆ alkyl, wherein the C₁₋₆ alkyl is optionally substituted with one or more R⁵¹; and
a C₃₋₁₂ carbocycle and a 3- to 12-membered heterocycle, each of which is optionally substituted with one or more R⁵³;
or R^{1a} and R^{2a} are joined together with the atoms to which they are attached to form an optionally substituted 3- to 12-membered heterocycle, wherein the heterocycle is optionally substituted with one or more R⁵³;
or R^{1b} and R^{2b} are joined together with the atoms to which they are attached to form an optionally substituted 3- to 12-membered heterocycle, wherein the heterocycle is optionally substituted with one or more R³;
R³ is hydrogen, —OR³⁰, —SR³⁰, —C(O)N(R³⁰)₂, —N(R³⁰)C(O)R³⁰, —N(R³⁰)C(O)N(R³⁰)₂, —N(R³⁰)₂, —C(O)R³⁰, —C(O)OR³⁰, —OC(O)R³⁰, —NO₂, and —CN;
L¹ is selected from a bond, —C₁₋₁₀alkylene-, —C₂₋₁₀alkenylene-, —C₂₋₁₀alkynylene-, —C₁₋₆alkylene-O—C₁₋₆alkylene-, —C₁₋₆alkylene-NH—C₁₋₆alkylene- C₃₋₆carbocycle, and —C₁₋₆ alkylene-(C₃₋₆carbocycle)-C₁₋₆alkylene-, wherein —C₁₋₁₀ alkylene-, —C₂₋₁₀ alkenylene-, —C₂₋₁₀ alkynylene-, C₃₋₆ carbocycle, and each C₁₋₆ alkylene group of —C₁₋₆alkylene-O—C₁₋₆alkylene-, —CC₁₋₆alkylene-NH—CC₁₋₆alkylene- and —C₁₋₆alkylene-(C₃₋₆carbocycle)-C₁₋₆ alkylene- are optionally substituted with one or more R⁵⁰;
L² is optionally substituted C₁₋₆ alkylene, wherein the C₁₋₆ alkylene is optionally substituted with one or more R⁵⁰;
Ring A¹ is either (a) an optionally substituted bridged, fused, or spirocyclic bicyclic heterocycle comprising at least one N atom and at least one O atom, wherein the heterocycle is optionally substituted with one or more R⁵³; or (b) a 3- to 12-membered heterocycle substituted with R⁴;
R⁴ is an optionally substituted 3- to 12-membered heterocycle, wherein the heterocycle is optionally substituted with one or more R⁵³;
R⁵ is selected from hydrogen, R⁶, —C(O)—C₁₋₆alkyl, —C(O)-heteroC₁₋₆alkyl, C₁₋₆ alkyl, and heteroC₁₋₆alkyl wherein the C₁₋₆ alkyl, either alone or part of another group, is optionally substituted with one or more R⁵⁰;
R⁶ is an amino acid residue;
each R³⁰ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more R⁵⁵;
R⁵⁰ is independently selected from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, C₃₋₁₂ carbocycle, and 3- to 12-membered heterocycle;
R⁵¹ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, optionally substituted C₃₋₁₂ carbocycle, and optionally substituted 3- to 12-membered heterocycle, wherein said optionally substituted C₃₋₁₂ carbocycle and optionally substituted 3- to 12-membered heterocycle are optionally substituted with one or more R₅₂;
R⁵² is independently selected at each occurrence from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), —CN, C₁₋₆ alkyl, C₂₋₆ alkenyl, and C₂₋₆ alkynyl;
R⁵³ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, an amino acid residue, optionally substituted C₁₋₆ alkyl, optionally substituted C₂₋₆ alkenyl, optionally substituted C₂₋₆ alkynyl, optionally substituted C₃₋₁₂ carbocycle, and optionally substituted 3- to 12-membered heterocycle, wherein said optionally substituted C₁₋₆ alkyl, optionally substituted C₂₋₆ alkenyl, and optionally substituted C₂₋₆ alkynyl are optionally substituted with one or more R⁵⁴ and said optionally substituted C₃₋₁₂ carbocycle and optionally substituted 3- to 12-membered heterocycle are optionally substituted with one or more R⁵²;
R⁵⁴ is independently selected at each occurrence from halogen, —OR⁶¹, —SR⁶¹, —C(O)N(R⁶¹)₂,
—N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), and —CN;
R⁵⁵ is independently selected at each occurrence from halogen, —CN, —NO₂, —OH, —N(R⁶⁰)₂, —C(O)N(R⁶⁰)₂, ═O, ═S, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
R⁶⁰ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle; and
R⁶¹ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle.

2. A compound of Formula (II): 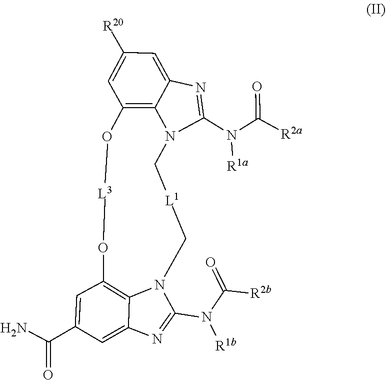
or a pharmaceutically acceptable salt or tautomer thereof;
wherein
R²⁰ is selected from hydrogen and —CON(R^{3a})(R^{3b});
R^{1a}, R^{1b}, R^{3a} and R^{3b} are independently selected from hydrogen and optionally substituted C₁₋₆ alkyl, wherein the C₁₋₆ alkyl is optionally substituted with one or more R⁵⁰;
R^{2a} and R^{2b} are independently selected from: (a) optionally substituted C₁₋₆ alkyl, wherein the C₁₋₆ alkyl is optionally substituted with one or more R⁵¹ and (b) C₃₋₁₂ carbocycle and 3- to 12-membered heterocycle, each of which is optionally substituted with one or more R⁵³;
or R^{1a} and R^{2a} are joined together with the atoms to which they are attached to form an optionally substituted 3- to 12-membered heterocycle, wherein the heterocycle is optionally substituted with one or more R⁵³;
or R^{1b} and R^{2b} are joined together with the atoms to which they are attached to form an optionally substituted 3- to 12-membered heterocycle, wherein the heterocycle is optionally substituted with one or more R⁵³;
L¹ is selected from a bond, —C₁₋₁₀alkylene-, —C₂₋₁₀alkenylene-, —C₂₋₁₀alkynylene-, —C₁₋₆alkylene-O—C₁₋₆alkylene-, —C₁₋₆-alkylene-NH—C₁₋₆alkylene-, C₃₋₆carbocycle, and —C₁₋₆ alkylene-(C₃₋₆carbocycle)-C₁₋₆ alkylene-, wherein —C₁₋₁₀ alkylene-, —C₂₋₁₀ alkenylene-, —C₂₋₁₀ alkylene-, C₃₋₆ carbocycle, and each C₁₋₆ alkylene group of —C₁₋₆alkylene-O—C₁₋₆ alkylene-, —C₁₋₆allkylene-NH—C₁₋₆alkylene- and —C₁₋₆alkylene-(C₃₋₆carbocycle)-C₁₋₆ alkylene- are optionally substituted with one or more R⁵⁰;
L³ is C₁₋₆ alkylene, which is substituted with either 1) one or more R⁵⁰ or 2) R^{8a} and R^{8b}, wherein
when L³ is substituted with R^{8a} and R^{8b}, R^{8a} and R^{8b} are joined together with the atoms to which they are attached to form an optionally substituted C₃₋₁₂ carbocycle or an optionally substituted 3- to 12-membered heterocycle, wherein said optionally substituted C₃₋₁₂ carbocycle and optionally substituted 3- to 12-membered heterocycle are optionally substituted with one or more R⁵²;
R⁵⁰ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, C₃₋₁₂ carbocycle, and 3- to 12-membered heterocycle;
R⁵¹ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, optionally substituted C₃₋₁₂ carbocycle, and optionally substituted 3- to 12-membered heterocycle, wherein said optionally substituted C₃₋₁₂ carbocycle and optionally substituted 3- to 12-membered heterocycle are optionally substituted with one or more R⁵²
R⁵² is independently selected at each occurrence from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), —CN, C₁₋₆ alkyl, C₂₋₆ alkenyl, and C₂₋₆ alkynyl;
R⁵³ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, an amino acid residue, optionally substituted C₁₋₆ alkyl, optionally substituted C₂₋₆ alkenyl, optionally substituted C₂₋₆ alkynyl, optionally substituted C₃₋₁₂ carbocycle, and optionally substituted 3- to 12-membered heterocycle, wherein said optionally substituted C₁₋₆ alkyl, optionally substituted C₂₋₆ alkenyl, and optionally substituted C₂₋₆ alkynyl are optionally substituted with one or more R⁵⁴ and said optionally substituted C₃₋₁₂ carbocycle and optionally substituted 3- to 12-membered heterocycle are optionally substituted with one or more R⁵²;
R⁵⁴ is independently selected at each occurrence from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), and —CN;
R⁶⁰ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle; and
R⁶¹ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle.

3. A compound of Formula (III): 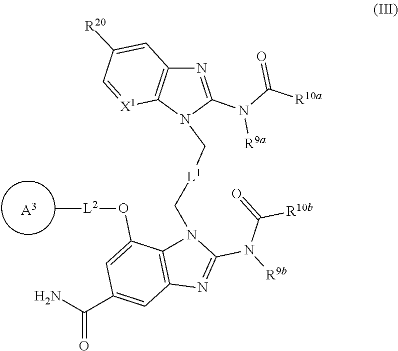
or a pharmaceutically acceptable salt thereof or tautomer thereof,
wherein
X¹ is selected from N and CR³;
R³ is selected from H, —OR³⁰, —SR³⁰, —C(O)N(R³⁰)₂, —N(R³⁰)C(O)R³⁰, —N(R³⁰)C(O)N(R³⁰)₂, —N(R³⁰)₂, —C(O)R³⁰, —C(O)OR³⁰, —OC(O)R³⁰, —NO₂, and —CN;
R^{3a} and R^{3b} are independently selected from hydrogen and optionally substituted C₁₋₆ alkyl, wherein the C₁₋₆ alkyl is optionally substituted with one or more R⁵⁰;
R^{9a} and R^{10a} are joined together with the atoms to which they are attached to form an optionally substituted 3- to 12-membered heterocycle, wherein the heterocycle is optionally substituted with one or more R⁵³;
R^{9b} and R^{10b} are joined together with the atoms to which they are attached to form an optionally substituted 3- to 12-membered heterocycle, wherein the heterocycle is optionally substituted with one or more R⁵³;
R²⁰ is selected from hydrogen and —CON(R^{3a})(R^{3b});
each R³⁰ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more R⁵;
L¹ is selected from a bond, —C₁₋₁₀alkylene-, —C₂₋₁₀alkenylene-, —C₂₋₁₀alkynylene-, —C₁₋₆alkylene-O—C₁₋₆alkylene-, —C₁₋₆alkylene-NH—C₁₋₆alkylene-, C₃₋₆carbocycle, and —C₁₋₆alkylene-(C₃₋₆carbocycle)-C₁₋₆alkylene, wherein —C₁₋₁₀ alkylene-, —C₂₋₁₀ alkenylene-, —C₂₋₁₀ alkynylene-, C₃₋₆ carbocycle, and each C₁₋₆ alkylene group of —C₁₋₆alkyl-O—C₁₋₆alkylene-, —C₁₋₆akylene-NH—C₁₋₆ alkylene-, and —C₁₋₆ alkylene-(C₃₋₆carbocycle)C₁₋₆alkylene- are optionally substituted with one or more R⁵⁰;
L² is optionally substituted —C₁₋₆ alkylene-, wherein the —C₁₋₆ alkylene- is optionally substituted with one or more R⁵⁰;
Ring A³ is C₃₋₁₂ carbocycle or 3- to 12-membered heterocycle, each of which is optionally substituted with one or more R⁵³;
R⁵⁰ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, C₃₋₁₂ carbocycle, and 3- to 12-membered heterocycle;
R⁵² is independently selected at each occurrence from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), —CN, C₁₋₆ alkyl, C₂₋₆ alkenyl, and C₂₋₆ alkynyl;
R⁵³ is independently selected at each occurrence from halogen, —OR⁶⁰, —SR⁶⁰, —C(O)N(R⁶⁰)₂, —N(R⁶⁰)C(O)R⁶⁰, —N(R⁶⁰)C(O)N(R⁶⁰)₂, —N(R⁶⁰)₂, —C(O)R⁶⁰, —C(O)OR⁶⁰, —OC(O)R⁶⁰, —NO₂, ═O, ═S, ═N(R⁶⁰), —CN, an amino acid residue, optionally substituted C₁₋₆ alkyl, optionally substituted C₂₋₆ alkenyl, optionally substituted C₂₋₆ alkynyl, optionally substituted C₃₋₁₂ carbocycle, and optionally substituted 3- to 12-membered heterocycle, wherein said optionally substituted C₁₋₆ alkyl, optionally substituted C₂₋₆ alkenyl, and optionally substituted C₂₋₆ alkynyl are optionally substituted with one or more R⁵⁴ and said optionally substituted C₃₋₁₂ carbocycle and optionally substituted 3- to 12-membered heterocycle are optionally substituted with one or more R⁵²;
R⁵⁴ is independently selected at each occurrence from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), and —CN;
R⁵⁵ is independently selected at each occurrence from halogen, —CN, —NO₂, —OH, —N(R⁶⁰)₂, —C(O)N(R⁶⁰)₂, ═O, ═S, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
R⁶⁰ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle; and
R⁶¹ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle.

4. The compound of claim 1, wherein R^{1a} and R^{1b} are both hydrogen.

5. The compound of claim 1, wherein R^{2a} and R^{2b} are independently an optionally substituted 3- to 12-membered heterocycle comprising at least one N atom.

6-13. (canceled)

14. The compound of claim 1, wherein L¹ is —C₂₋₁₀alkenyl optionally substituted with one or more R⁵⁰ or —C₁₋₁₀alkyl optionally substituted with one or more R⁵⁰.

15. (canceled)

16. (canceled)

17. The compound of claim 1, wherein X¹ is CH or X¹ is CR³ and R³ is OCH₃.

18-23. (canceled)

24. The compound of claim 1, wherein Ring A¹ is an optionally substituted 3- to 12-membered N-linked bridged, fused, or spirocyclic bicyclic heterocycle comprising at least one nitrogen atom, including the nitrogen bound to L², and at least one oxygen atom.

25. (canceled)

26. (canceled)

27. (canceled)

28. (canceled)

29. The compound of claim 1, wherein Ring A¹ is a N-linked 3- to 12-membered heterocycle substituted with R⁴.

30-47. (canceled)

48. The compound of claim 1, wherein the compound is selected from:

| Compound | |
|---|---|
| No. | Structure |
| 1 | 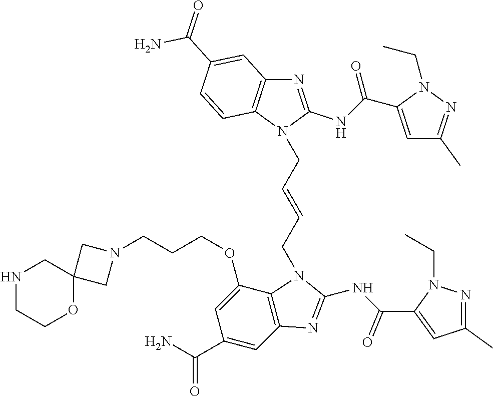 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(CNCCO4)C3)c21] |
| 2 | 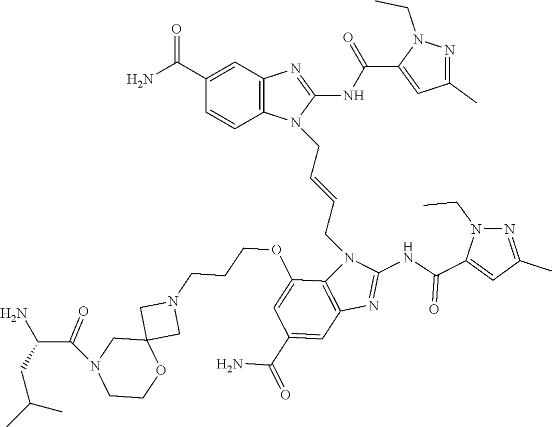 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@@H](N)CC(C)C)CCO4)c21] |
| 3 | 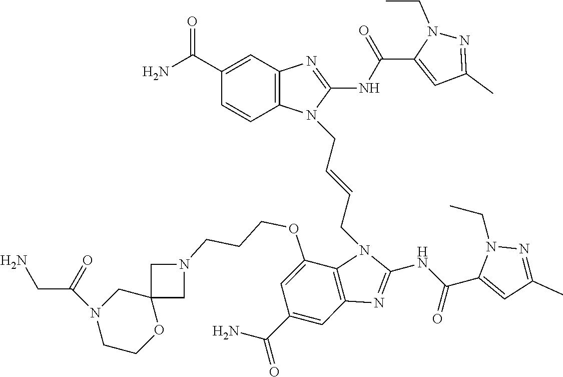 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)CN)CCO4)c21] |
| 4 | 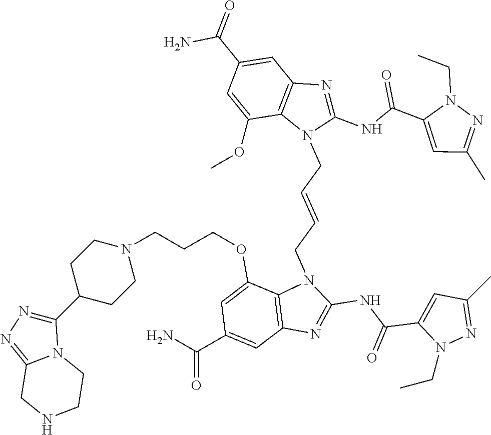 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCNC5)CC3)c21] |
| 5 | 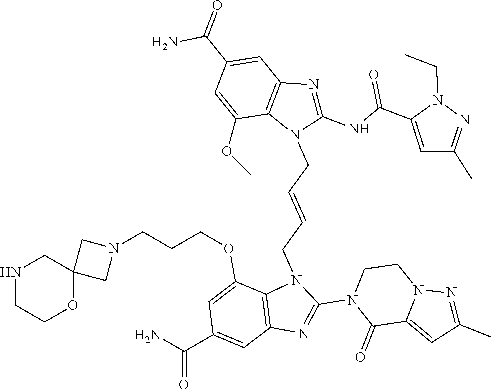 [SMILES: CCn1nc(C)cc1C(=O)Nc1nc2cc(C(N)=O)cc(OC)c2n1C/C=C/Cn1c(N2CCn3nc(C)cc3C2=O)nc2cc(C(N)=O)cc(OCCCN3CC4(CNCCO4)C3)c21] |
| 6 | 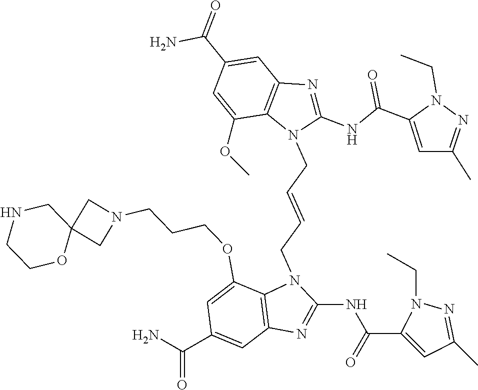 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(CNCCO4)C3)c21] |
| 7 | 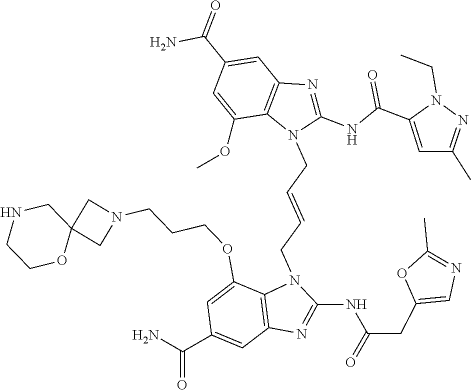 [SMILES: CCn1nc(C)cc1C(=O)Nc1nc2cc(C(N)=O)cc(OC)c2n1C/C=C/Cn1c(NC(=O)Cc2cnc(C)o2)nc2cc(C(N)=O)cc(OCCCN3CC4(CNCCO4)C3)c21] |
| 8 | 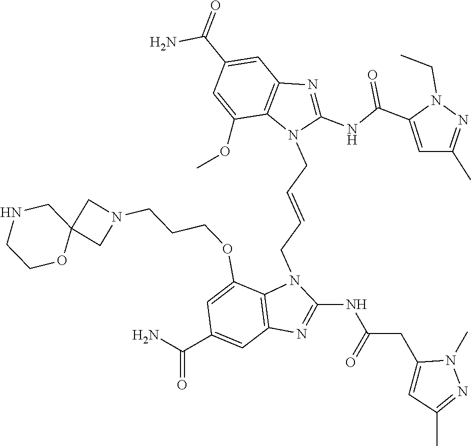 [SMILES: CCn1nc(C)cc1C(=O)Nc1nc2cc(C(N)=O)cc(OC)c2n1C/C=C/Cn1c(NC(=O)Cc2cc(C)nn2C)nc2cc(C(N)=O)cc(OCCCN3CC4(CNCCO4)C3)c21] |
| 9 | 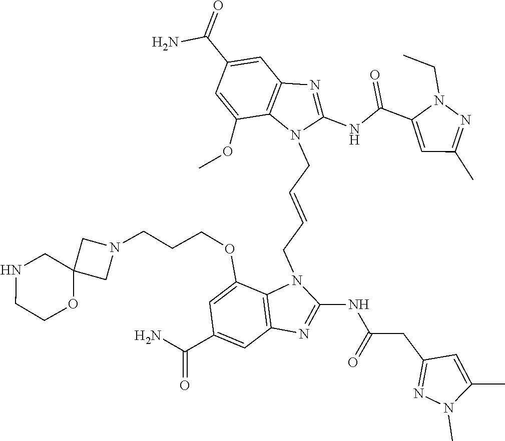 [SMILES: CCn1nc(C)cc1C(=O)Nc1nc2cc(C(N)=O)cc(OC)c2n1C/C=C/Cn1c(NC(=O)Cc2cc(C)n(C)n2)nc2cc(C(N)=O)cc(OCCCN3CC4(CNCCO4)C3)c21] |
| 10 | 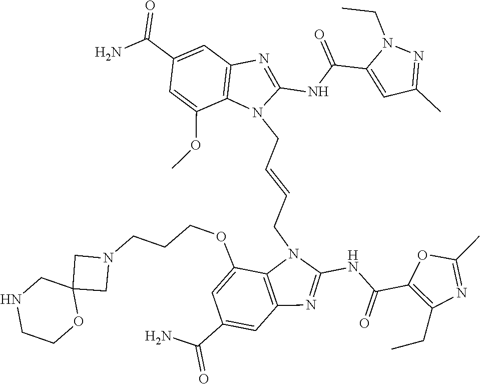 [SMILES: CCc1nc(C)oc1C(=O)Nc1nc2cc(C(N)=O)cc(OCCCN3CC4(CNCCO4)C3)c2n1C/C=C/Cn1c(NC(=O)c2cc(C)nn2CC)nc2cc(C(N)=O)cc(OC)c21] |
| 11 | 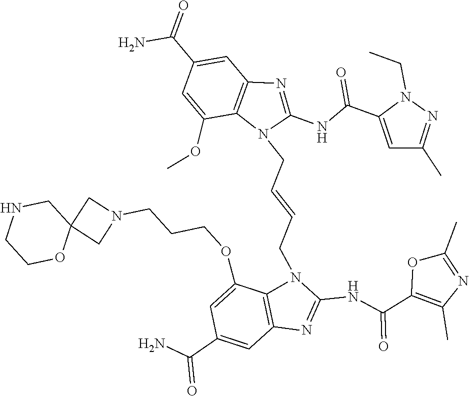 [SMILES: CCn1nc(C)cc1C(=O)Nc1nc2cc(C(N)=O)cc(OC)c2n1C/C=C/Cn1c(NC(=O)c2oc(C)nc2C)nc2cc(C(N)=O)cc(OCCCN3CC4(CNCCO4)C3)c21] |
| 12 | 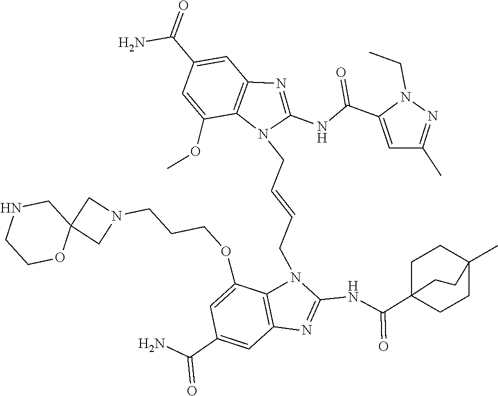 [SMILES: CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(CNCCO4)C3)c21] |
| 13 | 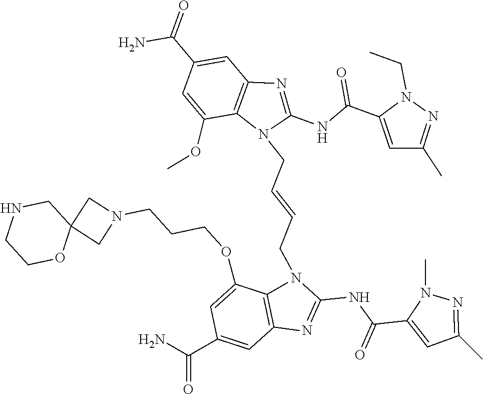 [SMILES: CCn1nc(C)cc1C(=O)Nc1nc2cc(C(N)=O)cc(OC)c2n1C/C=C/Cn1c(NC(=O)c2cc(C)nn2C)nc2cc(C(N)=O)cc(OCCCN3CC4(CNCCO4)C3)c21] |
| 14 | 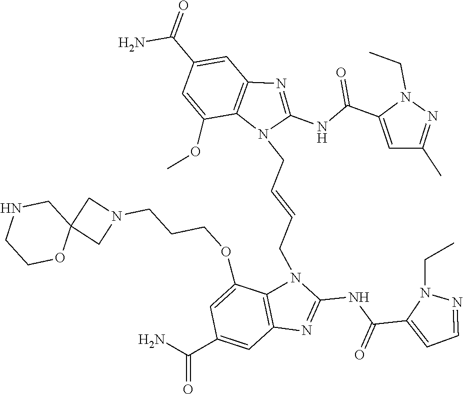 [SMILES: CCn1nc(C)cc1C(=O)Nc1nc2cc(C(N)=O)cc(OC)c2n1C/C=C/Cn1c(NC(=O)c2ccnn2CC)nc2cc(C(N)=O)cc(OCCCN3CC4(CNCCO4)C3)c21] |
| 15 | 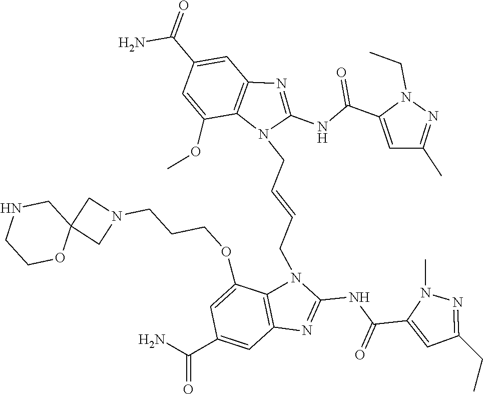 [SMILES: CCc1cc(C(=O)Nc2nc3cc(C(N)=O)cc(OCCCN4CC5(CNCCO5)C4)c3n2C/C=C/Cn2c(NC(=O)c3cc(C)nn3CC)nc3cc(C(N)=O)cc(OC)c32)n(C)n1] |
| 16 | 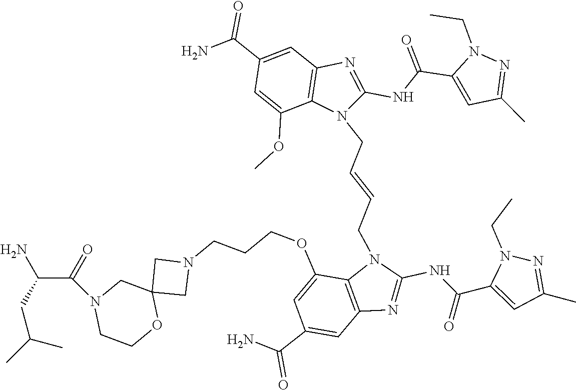 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(C3)CN(C(=O)[C@@H](N)CC(C)C)CCO4)c21] |
| 17 | 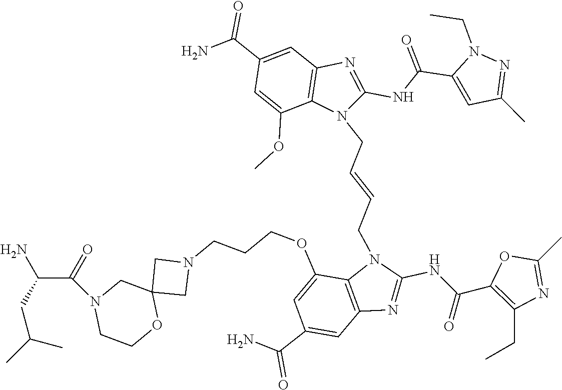 [SMILES: CCc1nc(C)oc1C(=O)Nc1nc2cc(C(N)=O)cc(OCCCN3CC4(C3)CN(C(=O)[C@@H](N)CC(C)C)CCO4)c2n1C/C=C/Cn1c(NC(=O)c2cc(C)nn2CC)nc2cc(C(N)=O)cc(OC)c21] |
| 18 | 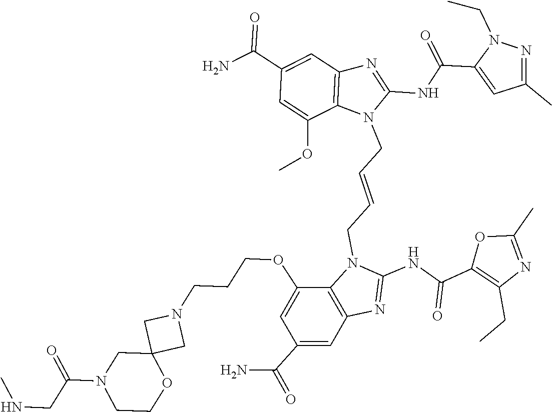 |
| 19 | 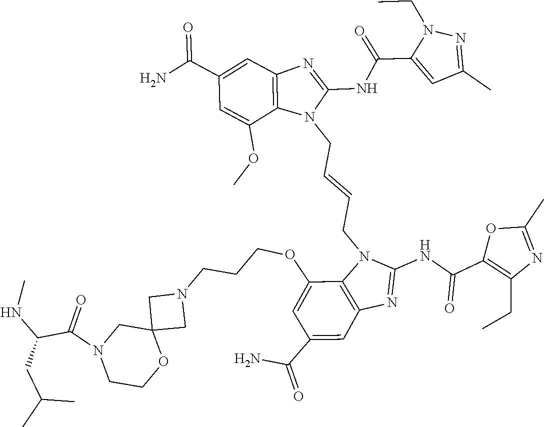 [SMILES: CCc1nc(C)oc1C(=O)Nc1nc2cc(C(N)=O)cc(OCCCN3CC4(C3)CN(C(=O)[C@H](CC(C)C)NC)CCO4)c2n1C/C=C/Cn1c(NC(=O)c2cc(C)nn2CC)nc2cc(C(N)=O)cc(OC)c21] |
| 20 | 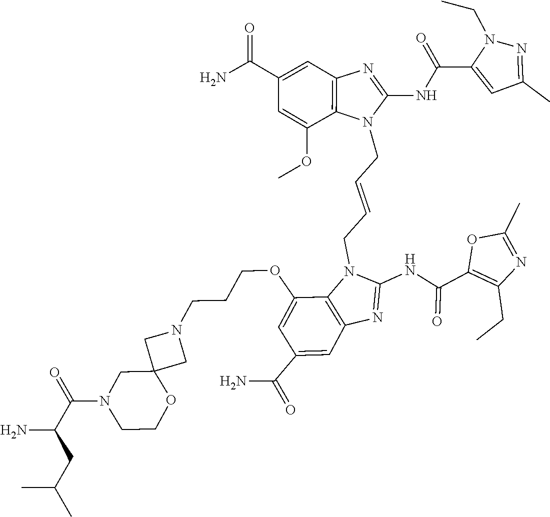 [SMILES: CCc1nc(C)oc1C(=O)Nc1nc2cc(C(N)=O)cc(OCCCN3CC4(C3)CN(C(=O)[C@H](N)CC(C)C)CCO4)c2n1C/C=C/Cn1c(NC(=O)c2cc(C)nn2CC)nc2cc(C(N)=O)cc(OC)c21] |
| 21 | 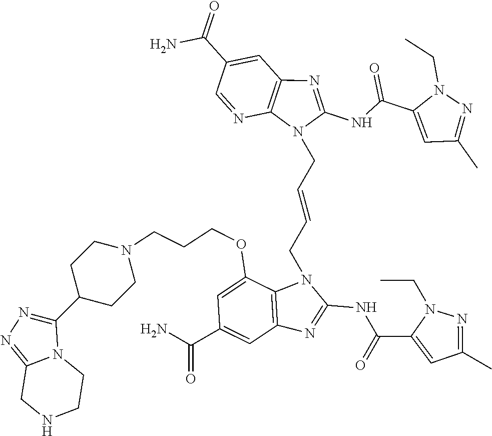 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCNC5)CC3)c21] |
| 22 | 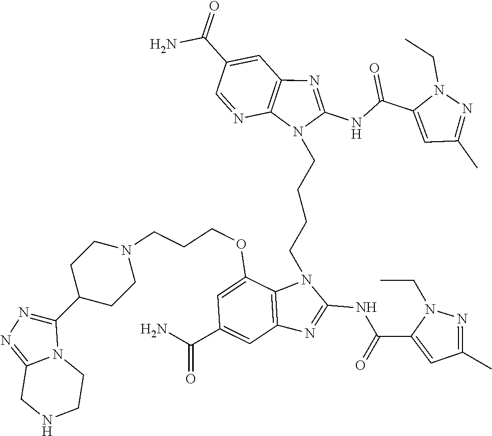 [SMILES: CCn1nc(C)cc1C(=O)Nc1nc2cc(C(N)=O)cnc2n1CCCCn1c(NC(=O)c2cc(C)nn2CC)nc2cc(C(N)=O)cc(OCCCN3CCC(c4nnc5n4CCNC5)CC3)c21] |
| 23 | 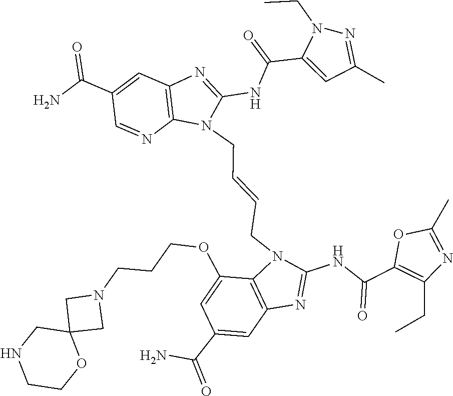 [SMILES: CCc1nc(C)oc1C(=O)Nc1nc2cc(C(N)=O)cc(OCCCN3CC4(CNCCO4)C3)c2n1C/C=C/Cn1c(NC(=O)c2cc(C)nn2CC)nc2cc(C(N)=O)cnc21] |
| 27 | 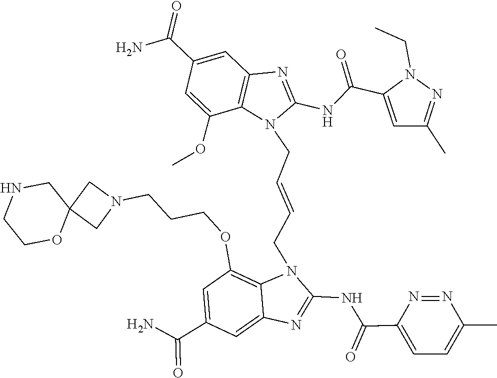 [SMILES: CCn1nc(C)cc1C(=O)Nc1nc2cc(C(N)=O)cc(OC)c2n1C/C=C/Cn1c(NC(=O)c2ccc(C)nn2)nc2cc(C(N)=O)cc(OCCCN3CC4(CNCCO4)C3)c21] |
| 28 | 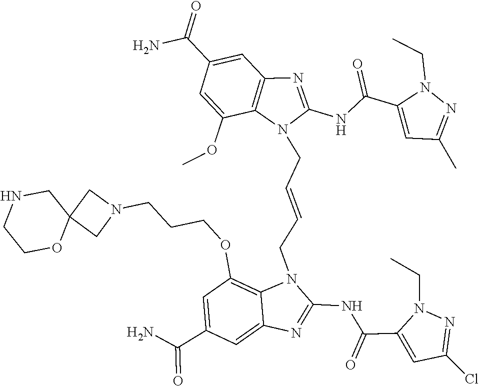 [SMILES: CCn1nc(C)cc1C(=O)Nc1nc2cc(C(N)=O)cc(OC)c2n1C/C=C/Cn1c(NC(=O)c2cc(Cl)nn2CC)nc2cc(C(N)=O)cc(OCCCN3CC4(CNCCO4)C3)c21] |
| 29 | 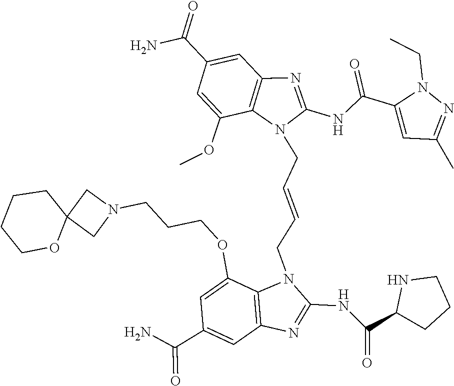 [SMILES: CCn1nc(C)cc1C(=O)Nc1nc2cc(C(N)=O)cc(OC)c2n1C/C=C/Cn1c(NC(=O)[C@@H]2CCCN2)nc2cc(C(N)=O)cc(OCCCN3CC4(CCCCO4)C3)c21] |
| 30 | 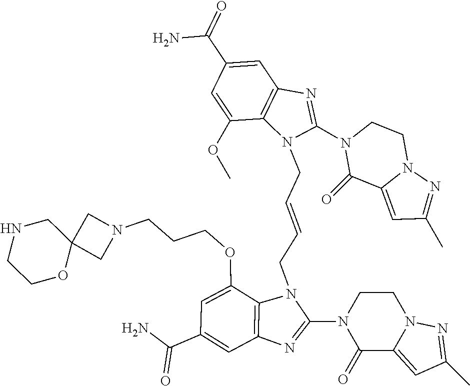 [SMILES: COc1cc(C(N)=O)cc2nc(N3CCn4nc(C)cc4C3=O)n(C/C=C/Cn3c(N4CCn5nc(C)cc5C4=O)nc4cc(C(N)=O)cc(OCCCN5CC6(CNCCO6)C5)c43)c12] |
| 32 | 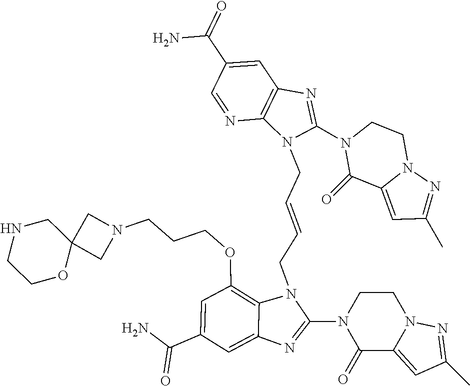 [SMILES: Cc1cc2n(n1)CCN(c1nc3cc(C(N)=O)cnc3n1C/C=C/Cn1c(N3CCn4nc(C)cc4C3=O)nc3cc(C(N)=O)cc(OCCCN4CC5(CNCCO5)C4)c31)C2=O] |
| 35 | 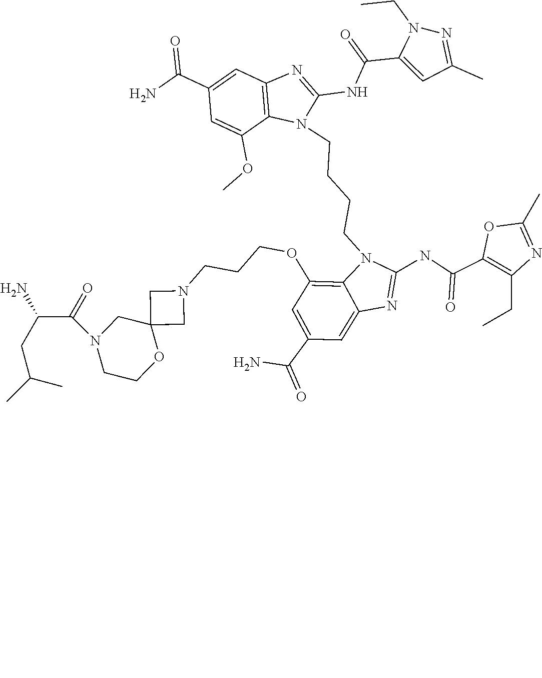 [SMILES: CCc1nc(C)oc1C(=O)Nc1nc2cc(C(N)=O)cc(OCCCN3CC4(C3)CN(C(=O)[C@@H](N)CC(C)C)CCO4)c2n1CCCCn1c(NC(=O)c2cc(C)nn2CC)nc2cc(C(N)=O)cc(OC)c21] |
| 36 | 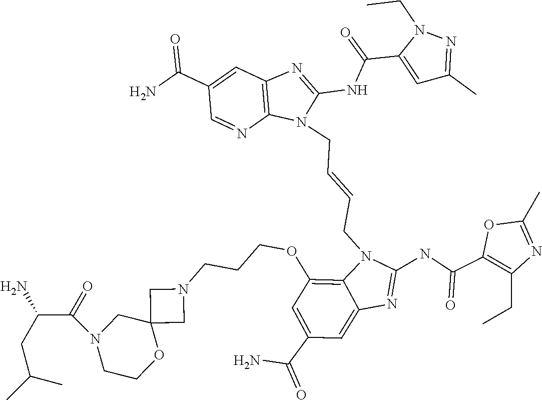 [SMILES: CCc1nc(C)oc1C(=O)Nc1nc2cc(C(N)=O)cc(OCCCN3CC4(C3)CN(C(=O)[C@@H](N)CC(C)C)CCO4)c2n1C/C=C/Cn1c(NC(=O)c2cc(C)nn2CC)nc2cc(C(N)=O)cnc21] |
| 37 | 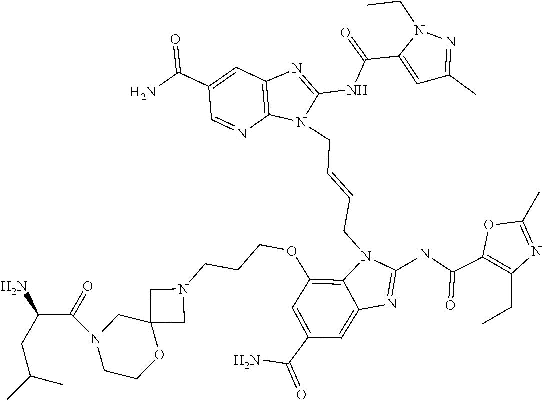 [SMILES: CCc1nc(C)oc1C(=O)Nc1nc2cc(C(N)=O)cc(OCCCN3CC4(C3)CN(C(=O)[C@H](N)CC(C)C)CCO4)c2n1C/C=C/Cn1c(NC(=O)c2cc(C)nn2CC)nc2cc(C(N)=O)cnc21] |
| 38 | 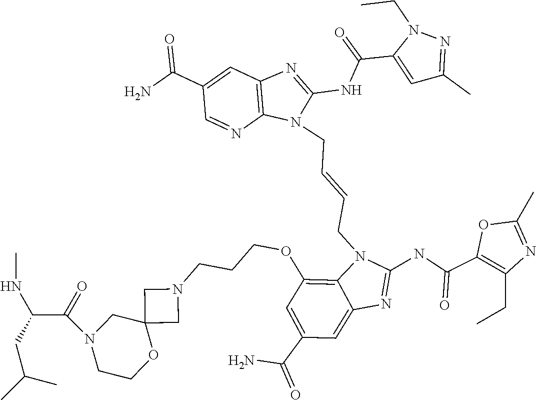 [SMILES: CCc1nc(C)oc1C(=O)Nc1nc2cc(C(N)=O)cc(OCCCN3CC4(C3)CN(C(=O)[C@H](CC(C)C)NC)CCO4)c2n1C/C=C/Cn1c(NC(=O)c2cc(C)nn2CC)nc2cc(C(N)=O)cnc21] |
| 39 | 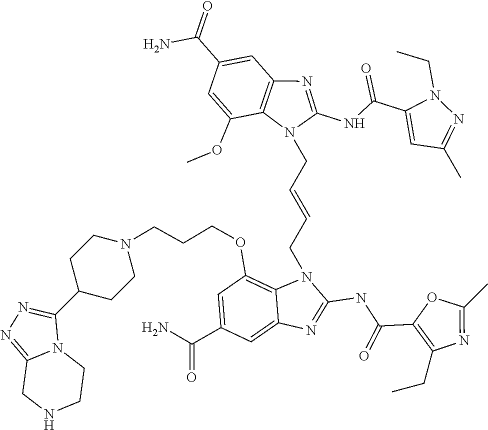 [SMILES: CCc1nc(C)oc1C(=O)Nc1nc2cc(C(N)=O)cc(OCCCN3CCC(c4nnc5n4CCNC5)CC3)c2n1C/C=C/Cn1c(NC(=O)c2cc(C)nn2CC)nc2cc(C(N)=O)cc(OC)c21] |
| 40 | 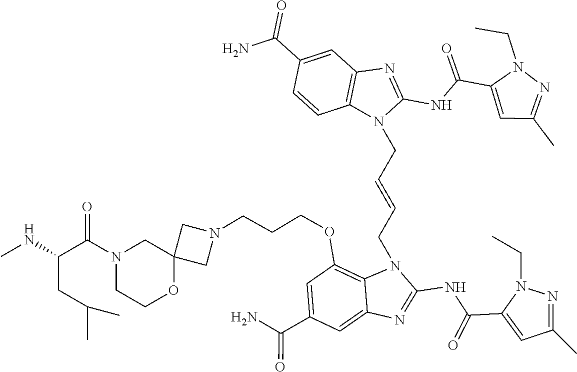 |
| 41 | 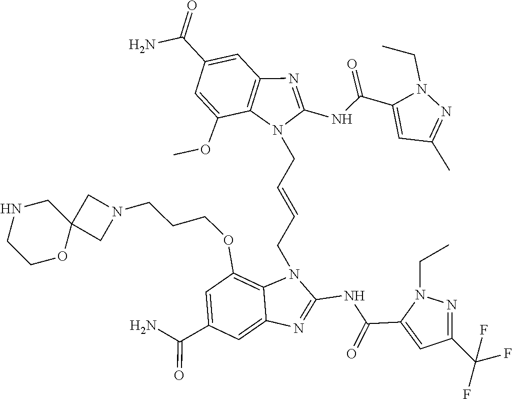 [SMILES: CCn1nc(C)cc1C(=O)Nc1nc2cc(C(N)=O)cc(OC)c2n1C/C=C/Cn1c(NC(=O)c2cc(C(F)(F)F)nn2CC)nc2cc(C(N)=O)cc(OCCCN3CC4(CNCCO4)C3)c21] |
| 42 | 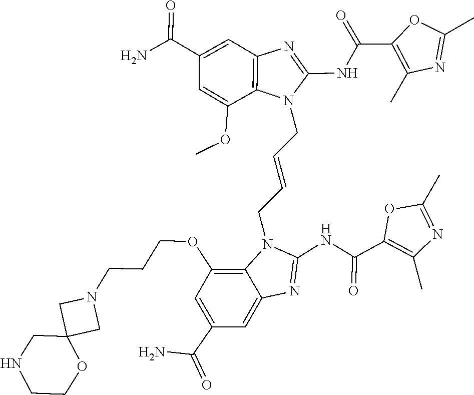 [SMILES: COc1cc(C(N)=O)cc2nc(NC(=O)c3oc(C)nc3C)n(C/C=C/Cn3c(NC(=O)c4oc(C)nc4C)nc4cc(C(N)=O)cc(OCCCN5CC6(CNCCO6)C5)c43)c12] |
| 43 | 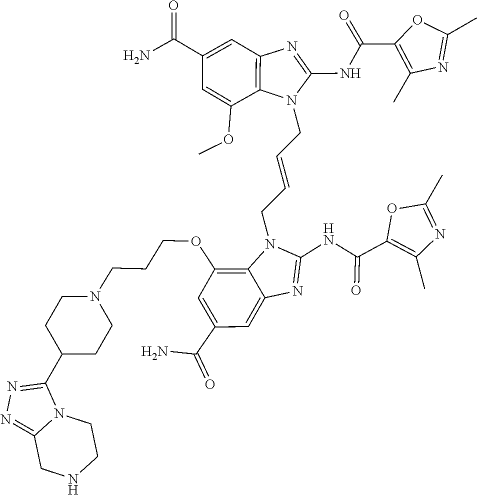 [SMILES: COc1cc(C(N)=O)cc2nc(NC(=O)c3oc(C)nc3C)n(C/C=C/Cn3c(NC(=O)c4oc(C)nc4C)nc4cc(C(N)=O)cc(OCCCN5CCC(c6nnc7n6CCNC7)CC5)c43)c12] |
| 44 | 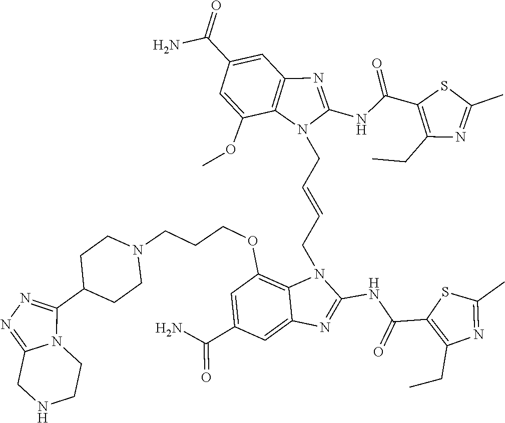 [SMILES: CCc1nc(C)sc1C(=O)Nc1nc2cc(C(N)=O)cc(OC)c2n1C/C=C/Cn1c(NC(=O)c2sc(C)nc2CC)nc2cc(C(N)=O)cc(OCCCN3CCC(c4nnc5n4CCNC5)CC3)c21] |
| 45 | 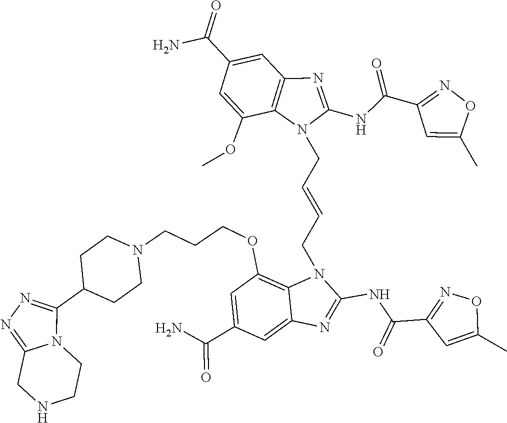 [SMILES: COc1cc(C(N)=O)cc2nc(NC(=O)c3cc(C)on3)n(C/C=C/Cn3c(NC(=O)c4cc(C)on4)nc4cc(C(N)=O)cc(OCCCN5CCC(c6nnc7n6CCNC7)CC5)c43)c12] |
| 46 | 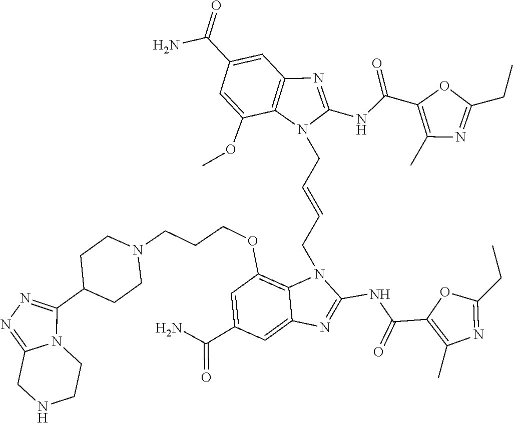 [SMILES: CCc1nc(C)c(C(=O)Nc2nc3cc(C(N)=O)cc(OC)c3n2C/C=C/Cn2c(NC(=O)c3oc(CC)nc3C)nc3cc(C(N)=O)cc(OCCCN4CCC(c5nnc6n5CCNC6)CC4)c32)o1] |
| 47 | 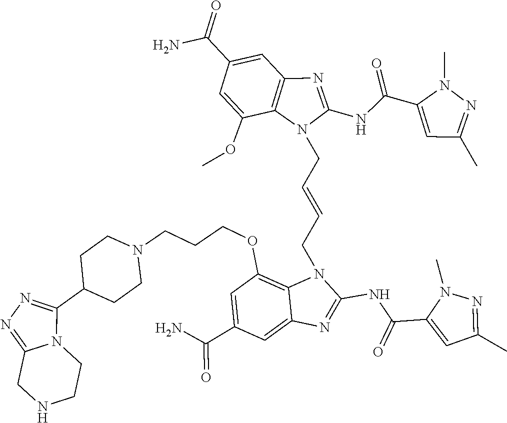 [SMILES: COc1cc(C(N)=O)cc2nc(NC(=O)c3cc(C)nn3C)n(C/C=C/Cn3c(NC(=O)c4cc(C)nn4C)nc4cc(C(N)=O)cc(OCCCN5CCC(c6nnc7n6CCNC7)CC5)c43)c12] |
| 49 | 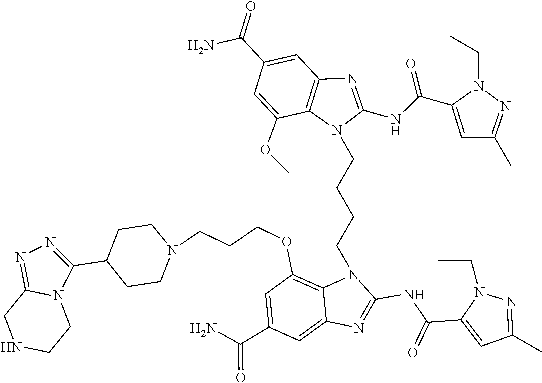 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCNC5)CC3)c21] |
| 54 | 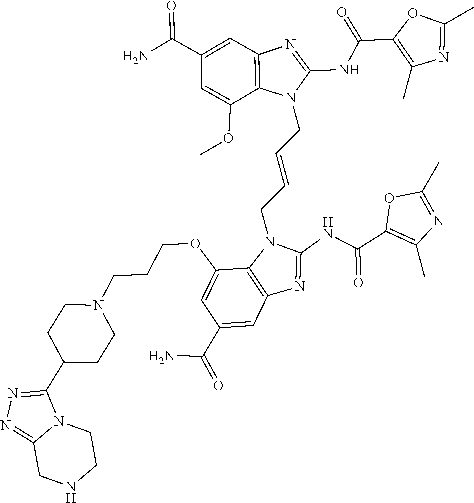 [SMILES: COc1cc(C(N)=O)cc2nc(NC(=O)c3oc(C)nc3C)n(C/C=C/Cn3c(NC(=O)c4oc(C)nc4C)nc4cc(C(N)=O)cc(OCCCN5CCC(c6nnc7n6CCNC7)CC5)c43)c12] |
| 56 | 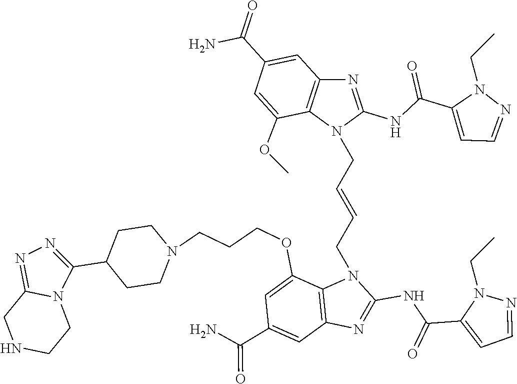 [SMILES: CCn1nccc1C(=O)Nc1nc2cc(C(N)=O)cc(OC)c2n1C/C=C/Cn1c(NC(=O)c2ccnn2CC)nc2cc(C(N)=O)cc(OCCCN3CCC(c4nnc5n4CCNC5)CC3)c21] |
| 57 | 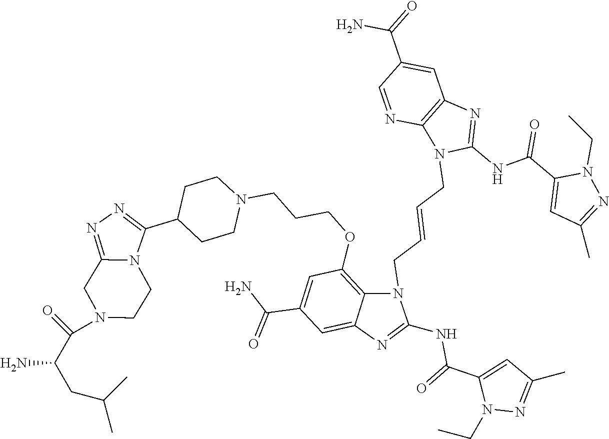 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)[C@@H](N)CC(C)C)C5)CC3)c21] |
| 58 | 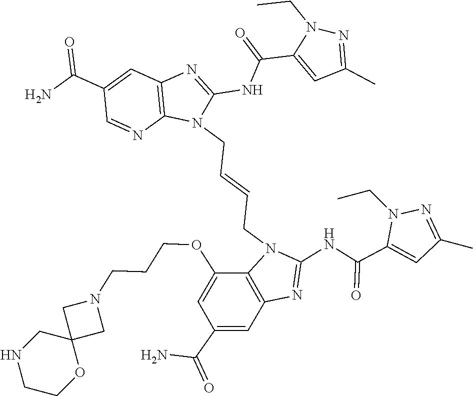 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CC4(CNCCO4)C3)c21] |
| 59 | 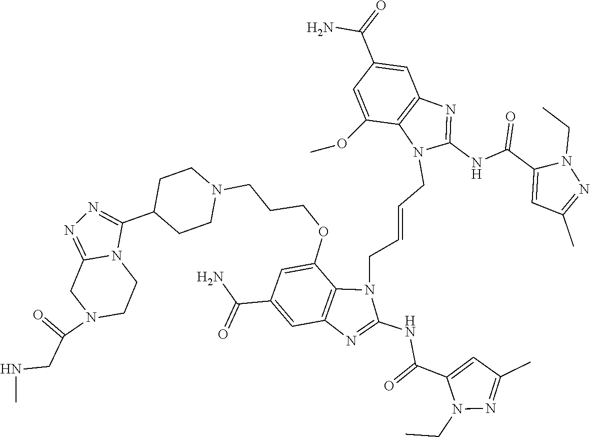 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CNC)C5)CC3)c21] |
| 60 | 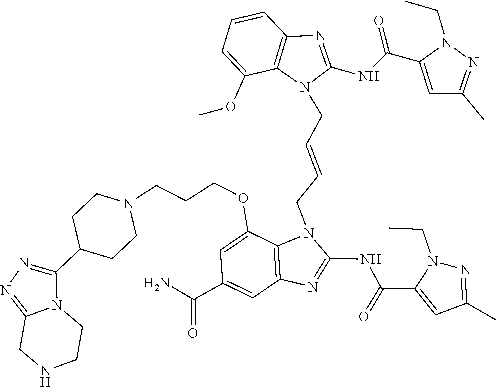 [SMILES: CCn1nc(C)cc1C(=O)Nc1nc2cccc(OC)c2n1C/C=C/Cn1c(NC(=O)c2cc(C)nn2CC)nc2cc(C(N)=O)cc(OCCCN3CCC(c4nnc5n4CCNC5)CC3)c21] |
| 61 | 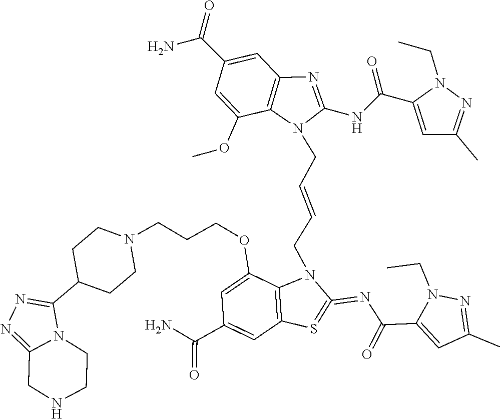 |
| 62 | 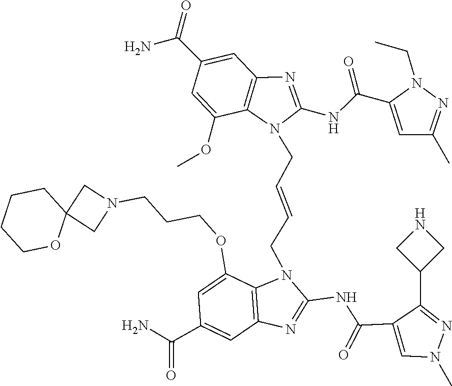 [SMILES: CCn1nc(C)cc1C(=O)Nc1nc2cc(C(N)=O)cc(OC)c2n1C/C=C/Cn1c(NC(=O)c2cn(C)nc2C2CNC2)nc2cc(C(N)=O)cc(OCCCN3CC4(CCCCO4)C3)c21] |
| 63 | 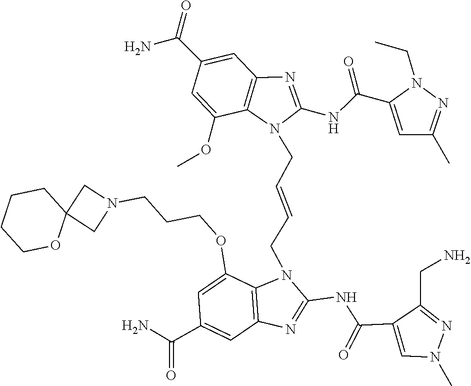 [SMILES: CCn1nc(C)cc1C(=O)Nc1nc2cc(C(N)=O)cc(OC)c2n1C/C=C/Cn1c(NC(=O)c2cn(C)nc2CN)nc2cc(C(N)=O)cc(OCCCN3CC4(CCCCO4)C3)c21] |
| 64 | 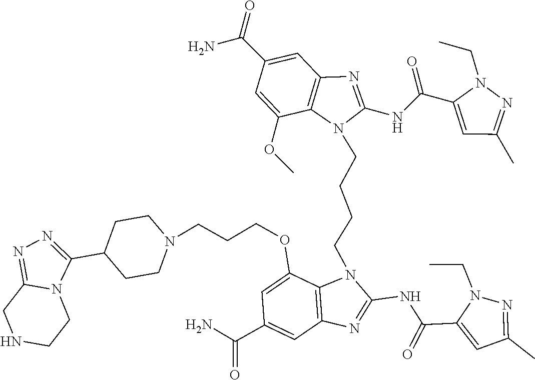 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCNC5)CC3)c21] |
| 65 | 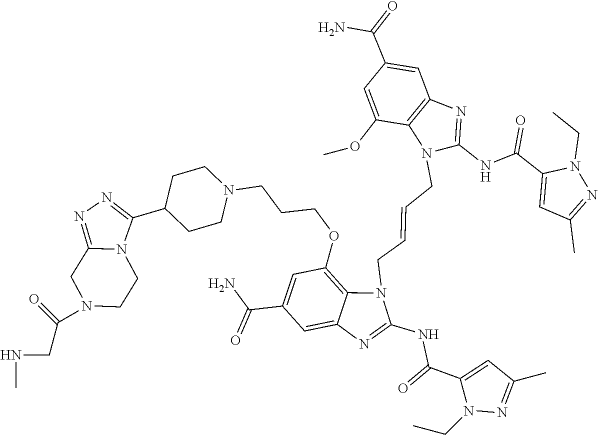 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CNC)C5)CC3)c21] |
| 66 | 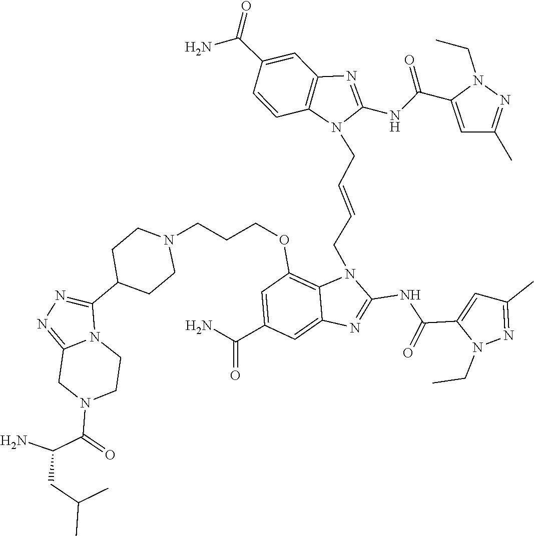 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)[C@@H](N)CC(C)C)C5)CC3)c21] |
| 67 | 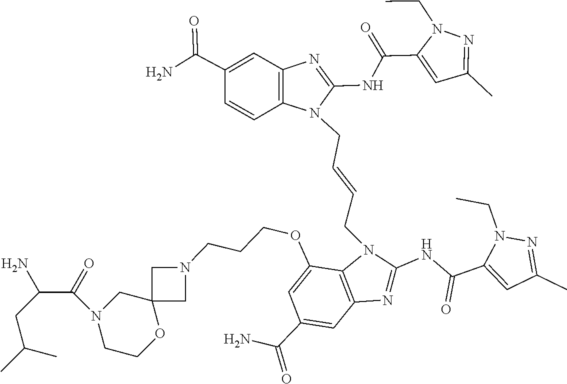 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)C(N)CC(C)C)CCO4)c21] |
| 68 | 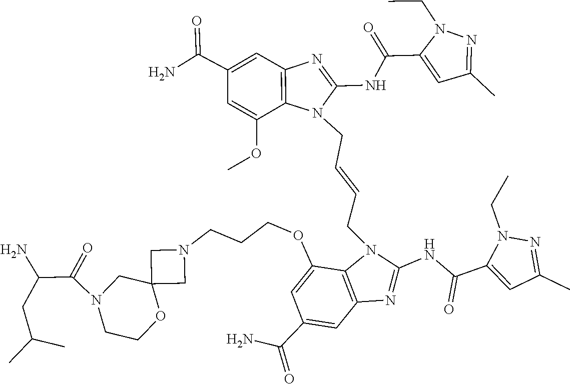 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(C3)CN(C(=O)C(N)CC(C)C)CCO4)c21] |
| 69 | 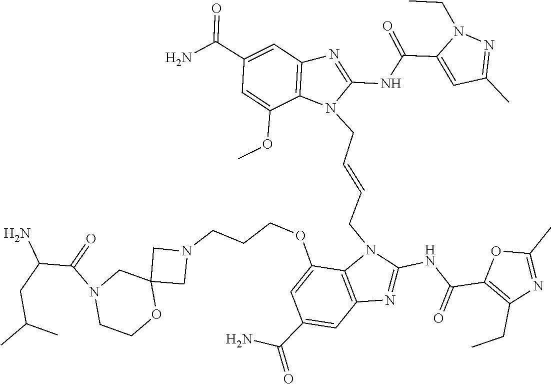 [SMILES: CCc1nc(C)oc1C(=O)Nc1nc2cc(C(N)=O)cc(OCCCN3CC4(C3)CN(C(=O)C(N)CC(C)C)CCO4)c2n1C/C=C/Cn1c(NC(=O)c2cc(C)nn2CC)nc2cc(C(N)=O)cc(OC)c21] |
| 70 | 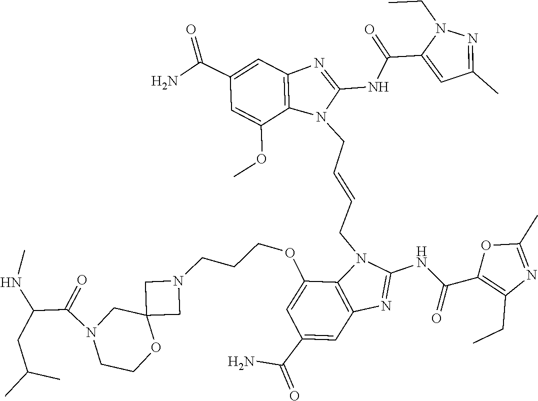 [SMILES: CCc1nc(C)oc1C(=O)Nc1nc2cc(C(N)=O)cc(OCCCN3CC4(C3)CN(C(=O)C(CC(C)C)NC)CCO4)c2n1C/C=C/Cn1c(NC(=O)c2cc(C)nn2CC)nc2cc(C(N)=O)cc(OC)c21] |
| 71 | 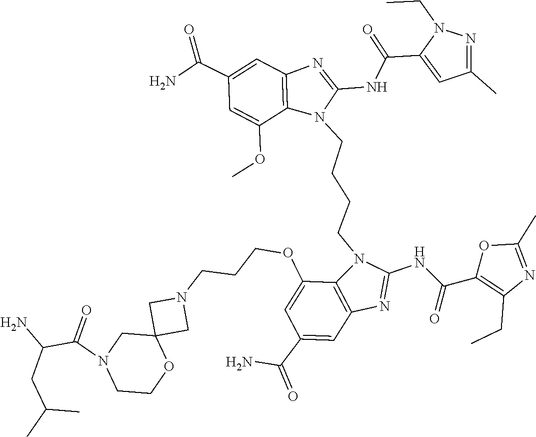 [SMILES: CCc1nc(C)oc1C(=O)Nc1nc2cc(C(N)=O)cc(OCCCN3CC4(C3)CN(C(=O)C(N)CC(C)C)CCO4)c2n1CCCCn1c(NC(=O)c2cc(C)nn2CC)nc2cc(C(N)=O)cc(OC)c21] |
| 72 | 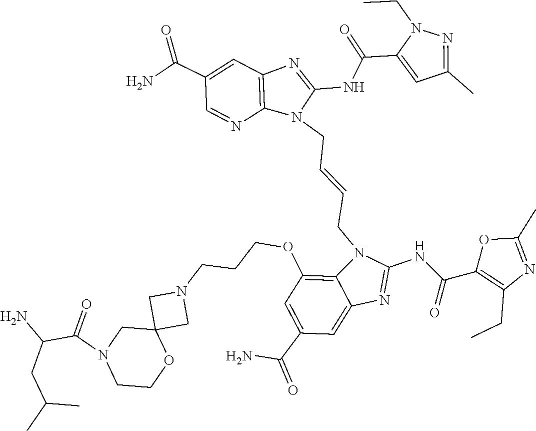 [SMILES: CCc1nc(C)oc1C(=O)Nc1nc2cc(C(N)=O)cc(OCCCN3CC4(C3)CN(C(=O)C(N)CC(C)C)CCO4)c2n1C/C=C/Cn1c(NC(=O)c2cc(C)nn2CC)nc2cc(C(N)=O)cnc21] |
| 73 | 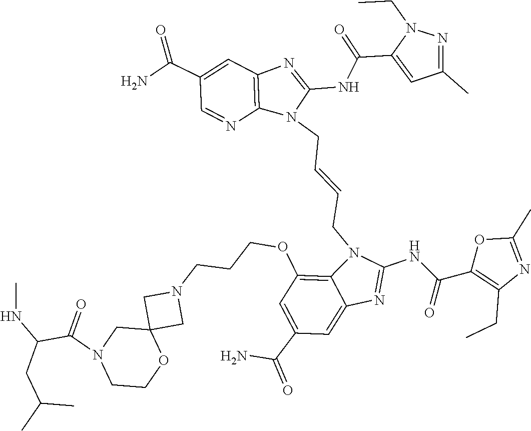 [SMILES: CCc1nc(C)oc1C(=O)Nc1nc2cc(C(N)=O)cc(OCCCN3CC4(C3)CN(C(=O)C(CC(C)C)NC)CCO4)c2n1C/C=C/Cn1c(NC(=O)c2cc(C)nn2CC)nc2cc(C(N)=O)cnc21] |
| 74 | 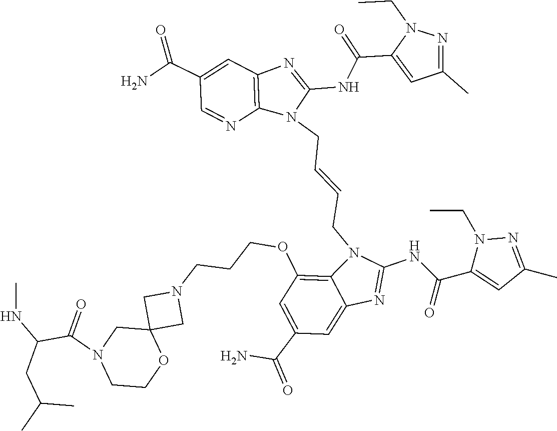 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CC4(C3)CN(C(=O)C(CC(C)C)NC)CCO4)c21] |
| 75 | 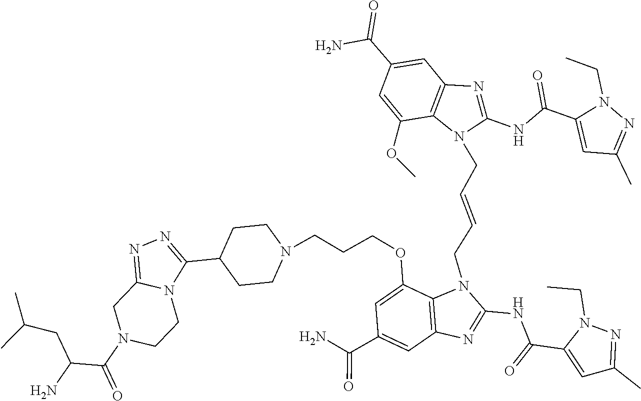 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)C(N)CC(C)C)C5)CC3)c21] |
| 76 | 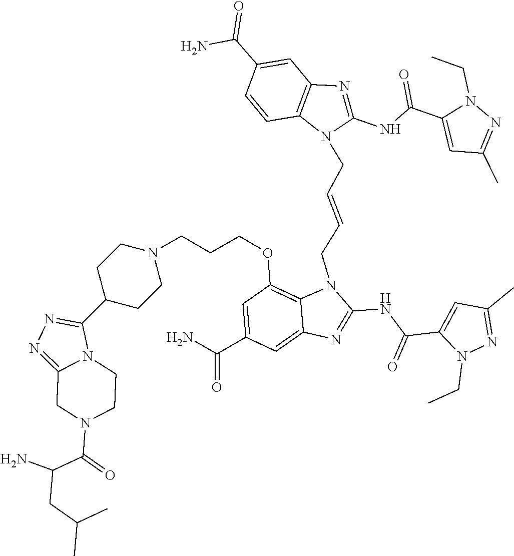 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)C(N)CC(C)C)C5)CC3)c21] |
| 77 | 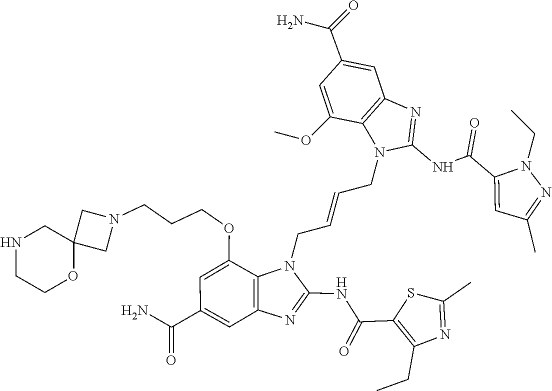 [SMILES: CCc1nc(C)sc1C(=O)Nc1nc2cc(C(N)=O)cc(OCCCN3CC4(CNCCO4)C3)c2n1C/C=C/Cn1c(NC(=O)c2cc(C)nn2CC)nc2cc(C(N)=O)cc(OC)c21] |
| 78 | 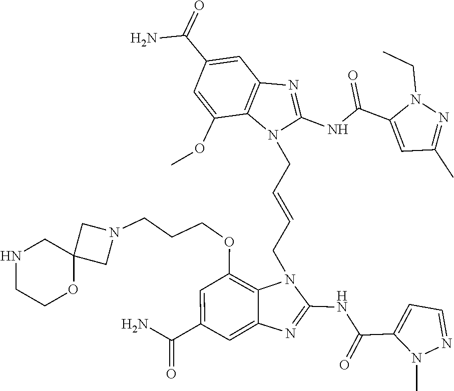 [SMILES: CCn1nc(C)cc1C(=O)Nc1nc2cc(C(N)=O)cc(OC)c2n1C/C=C/Cn1c(NC(=O)c2ccnn2C)nc2cc(C(N)=O)cc(OCCCN3CC4(CNCCO4)C3)c21] |
| 79 | 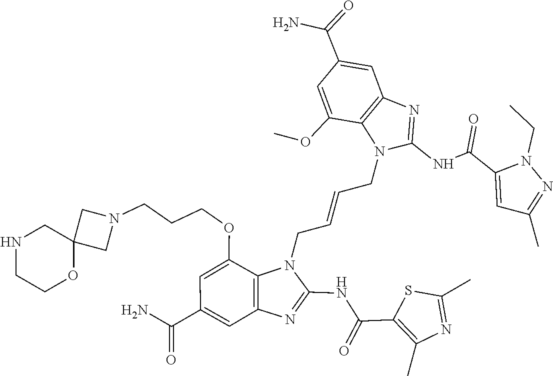 [SMILES: CCn1nc(C)cc1C(=O)Nc1nc2cc(C(N)=O)cc(OC)c2n1C/C=C/Cn1c(NC(=O)c2sc(C)nc2C)nc2cc(C(N)=O)cc(OCCCN3CC4(CNCCO4)C3)c21] |
| 80 | 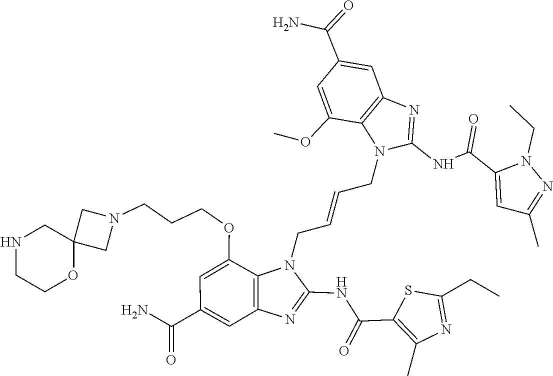 [SMILES: CCc1nc(C)c(C(=O)Nc2nc3cc(C(N)=O)cc(OCCCN4CC5(CNCCO5)C4)c3n2C/C=C/Cn2c(NC(=O)c3cc(C)nn3CC)nc3cc(C(N)=O)cc(OC)c32)s1] |
| 81 | 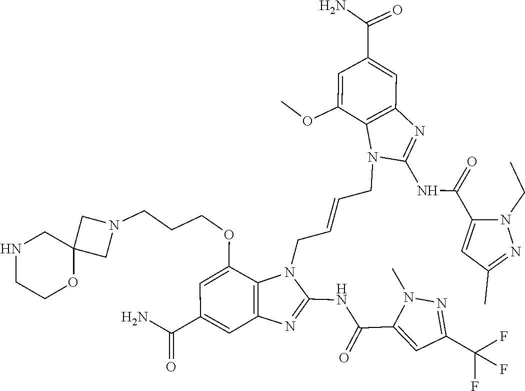 [SMILES: CCn1nc(C)cc1C(=O)Nc1nc2cc(C(N)=O)cc(OC)c2n1C/C=C/Cn1c(NC(=O)c2cc(C(F)(F)F)nn2C)nc2cc(C(N)=O)cc(OCCCN3CC4(CNCCO4)C3)c21] |
| 82 | 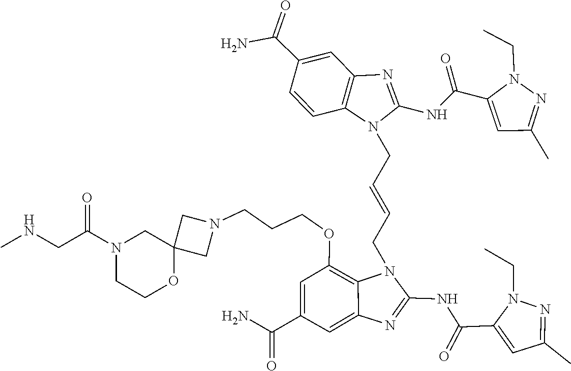 |
| 83 | 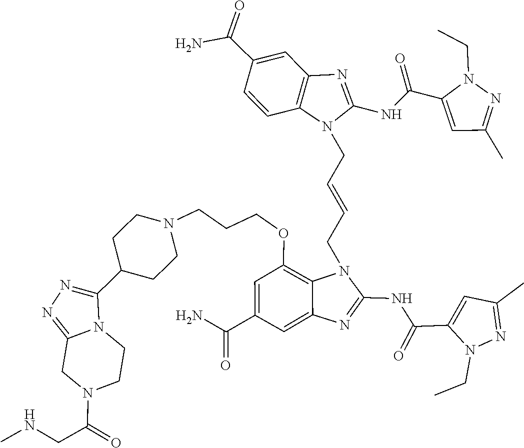 |
| 84 | 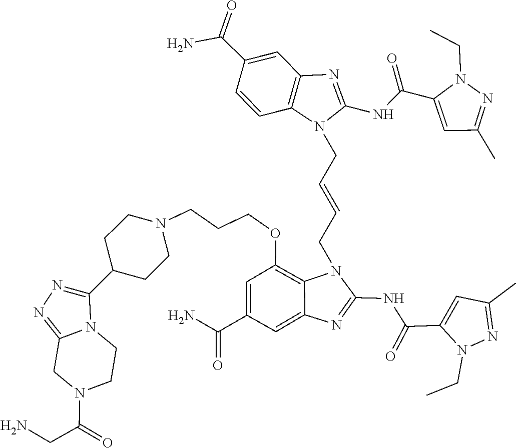 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)CN)C5)CC3)c21] |
| 85 | 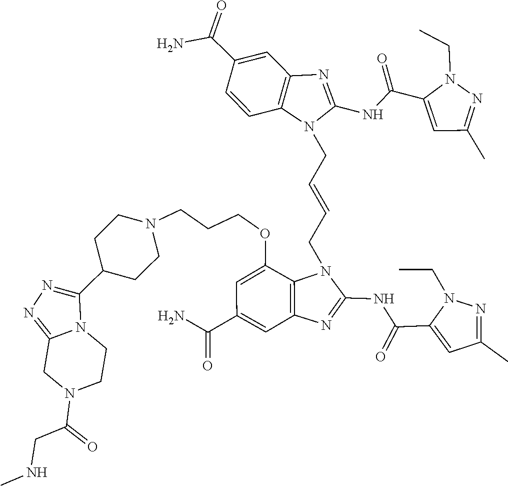 |
| 86 | 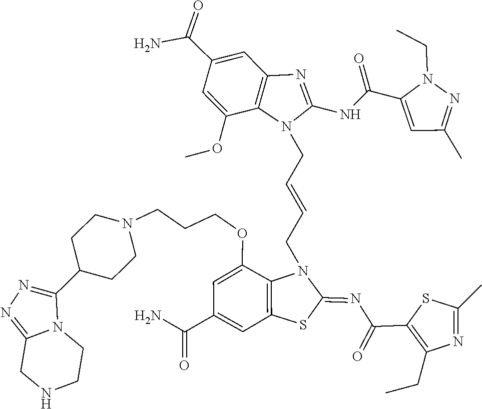 [SMILES: CCc1nc(C)sc1C(=O)/N=c1\sc2cc(C(N)=O)cc(OCCCN3CCC(c4nnc5n4CCNC5)CC3)c2n1C/C=C/Cn1c(NC(=O)c2cc(C)nn2CC)nc2cc(C(N)=O)cc(OC)c21] |
| 88 | 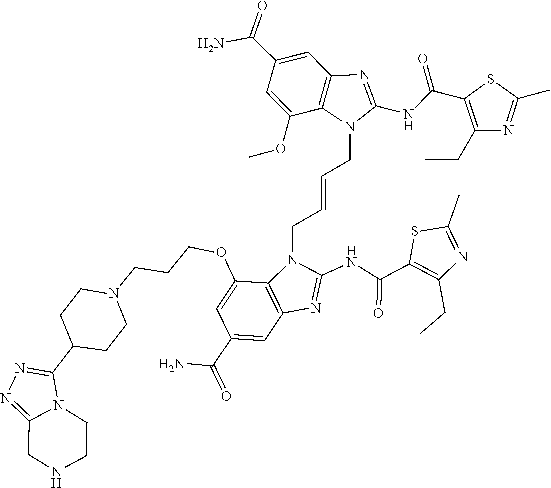 [SMILES: CCc1nc(C)sc1C(=O)Nc1nc2cc(C(N)=O)cc(OC)c2n1C/C=C/Cn1c(NC(=O)c2sc(C)nc2CC)nc2cc(C(N)=O)cc(OCCCN3CCC(c4nnc5n4CCNC5)CC3)c21] |
| 89 | 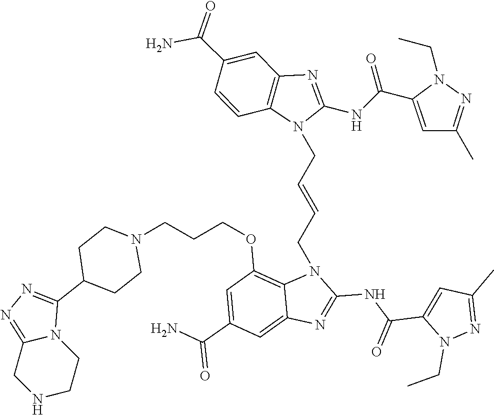 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCNC5)CC3)c21] |
| 90 | 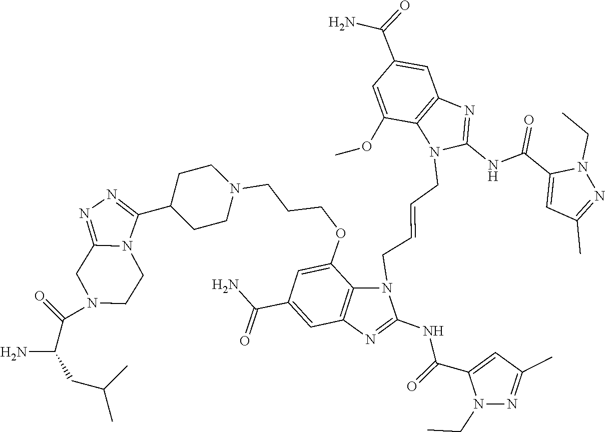 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@@H](N)CC(C)C)C5)CC3)c21] |

or a pharmaceutically acceptable salt or tautomer thereof.

49. The compound of claim 2, wherein the compound is selected from:

| Compound | |
|---|---|
| No. | Structure |
| 24 | 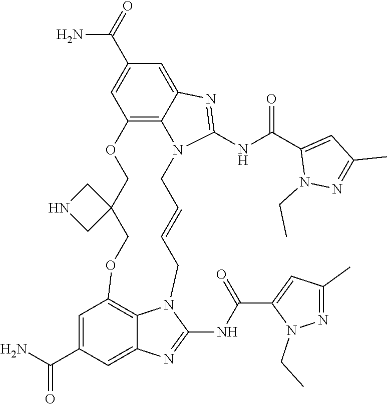 [SMILES: CCn1nc(C)cc1C(=O)Nc1nc2cc(C(N)=O)cc3c2n1C/C=C/Cn1c(NC(=O)c2cc(C)nn2CC)nc2cc(C(N)=O)cc(c21)OCC1(CNC1)CO3] |
| 25 | 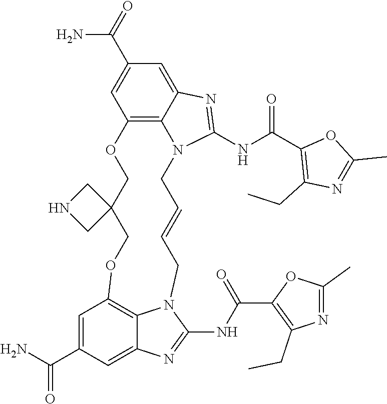 [SMILES: CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CNC1)CO3] |
| 26 | 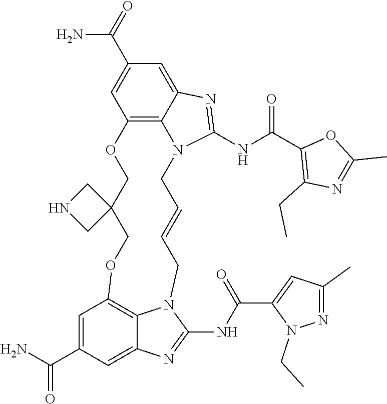 [SMILES: CCc1nc(C)oc1C(=O)Nc1nc2cc(C(N)=O)cc3c2n1C/C=C/Cn1c(NC(=O)c2cc(C)nn2CC)nc2cc(C(N)=O)cc(c21)OCC1(CNC1)CO3] |
| 34 | 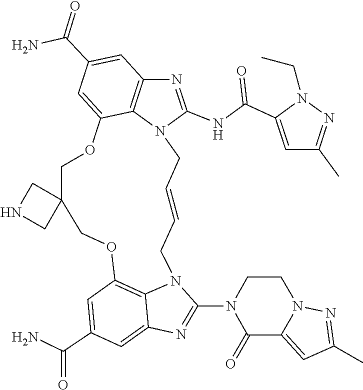 [SMILES: CCn1nc(C)cc1C(=O)Nc1nc2cc(C(N)=O)cc3c2n1C/C=C/Cn1c(N2CCn4nc(C)cc4C2=O)nc2cc(C(N)=O)cc(c21)OCC1(CNC1)CO3] |
| 48 | 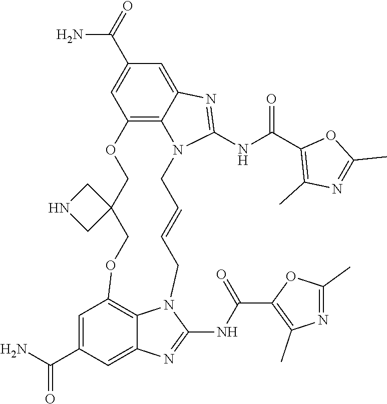 [SMILES: Cc1nc(C)c(C(=O)Nc2nc3cc(C(N)=O)cc4c3n2C/C=C/Cn2c(NC(=O)c3oc(C)nc3C)nc3cc(C(N)=O)cc(c32)OCC2(CNC2)CO4)o1] |
| 50 | 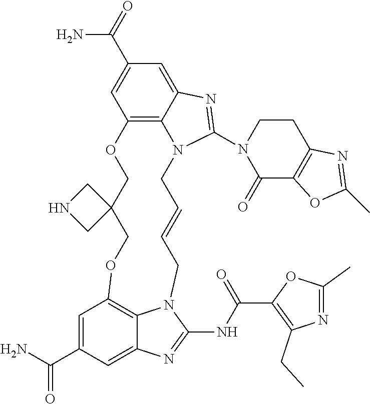 [SMILES: CCc1nc(C)oc1C(=O)Nc1nc2cc(C(N)=O)cc3c2n1C/C=C/Cn1c(N2CCc4nc(C)oc4C2=O)nc2cc(C(N)=O)cc(c21)OCC1(CNC1)CO3] |
| 51 | 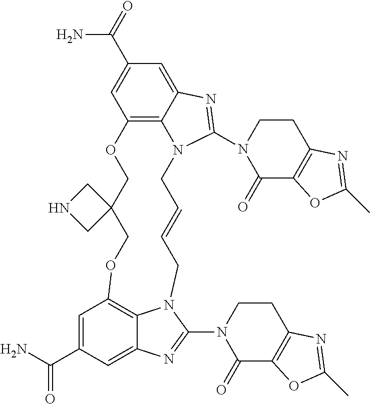 [SMILES: Cc1nc2c(o1)C(=O)N(c1nc3cc(C(N)=O)cc4c3n1C/C=C/Cn1c(N3CCc5nc(C)oc5C3=O)nc3cc(C(N)=O)cc(c31)OCC1(CNC1)CO4)CC2] |
| 52 | 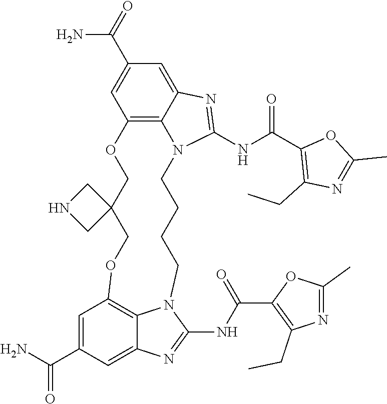 [SMILES: CCc1nc(C)oc1C(=O)Nc1nc2cc(C(N)=O)cc3c2n1CCCCn1c(NC(=O)c2oc(C)nc2CC)nc2cc(C(N)=O)cc(c21)OCC1(CNC1)CO3] |
| 59 | 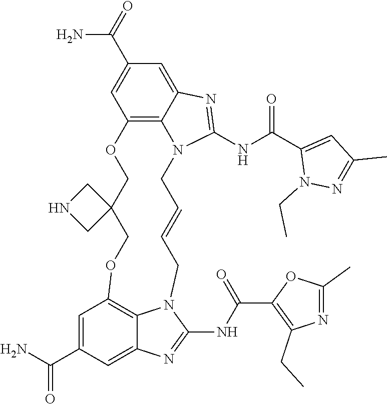 [SMILES: CCc1nc(C)oc1C(=O)Nc1nc2cc(C(N)=O)cc3c2n1C/C=C/Cn1c(NC(=O)c2cc(C)nn2CC)nc2cc(C(N)=O)cc(c21)OCC1(CNC1)CO3] |
| 87 | 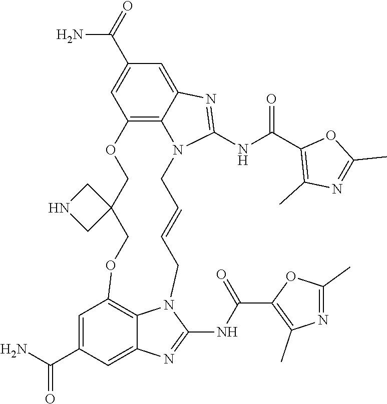 [SMILES: Cc1nc(C)c(C(=O)Nc2nc3cc(C(N)=O)cc4c3n2C/C=C/Cn2c(NC(=O)c3oc(C)nc3C)nc3cc(C(N)=O)cc(c32)OCC2(CNC2)CO4)o1] |

or a pharmaceutically acceptable salt or tautomer thereof.

50. The compound of claim 3, wherein the compound is selected from: 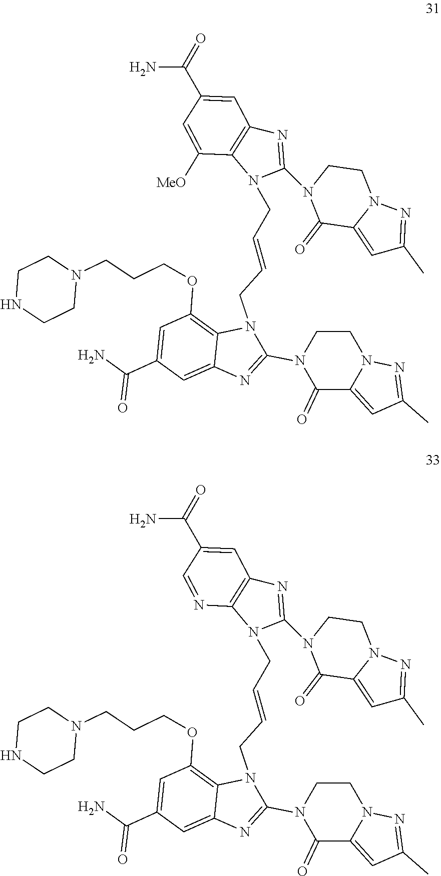 [SMILES: COc1cc(C(N)=O)cc2nc(N3CCn4nc(C)cc4C3=O)n(C/C=C/Cn3c(N4CCn5nc(C)cc5C4=O)nc4cc(C(N)=O)cc(OCCCN5CCNCC5)c43)c12 ; Cc1cc2n(n1)CCN(c1nc3cc(C(N)=O)cnc3n1C/C=C/Cn1c(N3CCn4nc(C)cc4C3=O)nc3cc(C(N)=O)cc(OCCCN4CCNCC4)c31)C2=O]
or a pharmaceutically acceptable salt or tautomer thereof.

51. A linker-payload compound comprising the compound of claim 1 linked to RG optionally via a linker, wherein RG is a reactive linker group and the linker comprises a protease cleavable linker, a pH sensitive linker, or a non-cleavable linker.

52. (canceled)

53. (canceled)

54. A linker-payload compound of claim 51, according to the structure of Formula (LP-I) or (LP-IV): 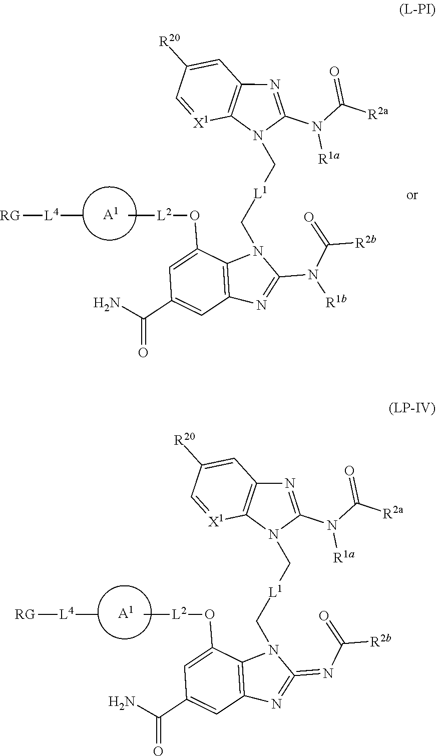
or a pharmaceutically acceptable salt or tautomer thereof,
wherein
L⁴ is a bond or a linker; and
RG is a reactive linker group.

55. A linker-payload compound of claim 51, according to the structure of Formula (LP-II): 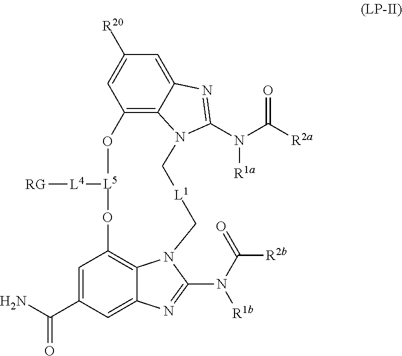
or a pharmaceutically acceptable salt or tautomer thereof,
wherein
L⁵ is a linker comprising —C₁₋₆ alkylene-, which is substituted with either 1) one R⁵⁶ or 2) R^{18a} and R^{18b}; and wherein the —C₁₋₆ alkylene- is optionally further substituted with one or more R⁵⁰, wherein
when L⁵ is substituted with R^{18a} and R^{18b}, R^{18a} and R^{18b} are joined together with the atoms to which they are attached to form a C₃₋₁₂ carbocycle or a 3- to 12-membered heterocycle, wherein said C₃₋₁₂ carbocycle and 3- to 12-membered heterocycle are attached to -L⁴-RG and further optionally substituted with one or more R⁵²;
R⁵⁶ is independently selected at each occurrence from —OR⁶², —SR⁶²,
—C(O)N(R⁶⁰)(R⁶²), —N(R⁶²)C(O)R⁶⁰, —N(R⁶⁰)C(O)R⁶², —N(R⁶²)C(O)N(R⁶⁰)₂, —N(R⁶⁰)C(O)N(R⁶⁰)(R⁶²), —N(R⁶⁰)(R⁶²), —C(O)R⁶²,
—C(O)OR⁶², —OC(O)R⁶², =N(R⁶²), C₃₋₁₂ carbocycle substituted with R⁶², and 3- to 12-membered heterocycle substituted with R⁶²;
R⁶² is selected from —C₁₋₁₀ alkylene- attached to -L⁴-RG, —C₂₋₁₀ alkenylene-attached to -L⁴-RG, —C₂₋₁₀ alkynylene- attached to -L⁴-RG, C₃₋₁₂ carbocyclene attached to -L⁴-RG, and 3- to 12-membered heterocyclene attached to -L⁴-RG; and
L⁴ is a bond or a linker; and
RG is a reactive linker group.

56. A linker-payload compound of claim 51, according to the structure of Formula (LP-III): 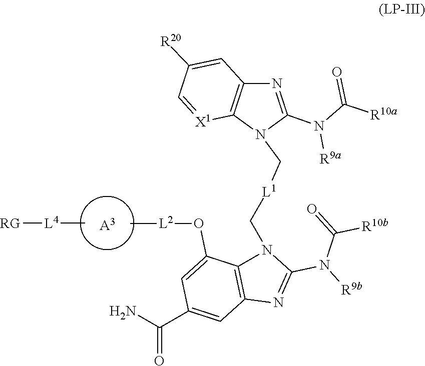
or a pharmaceutically acceptable salt or tautomer thereof,
wherein
L⁴ is a bond or a linker; and
RG is a reactive linker group.

57. A linker-payload compound of claim 51, according to the structure of Formula (LP-V): 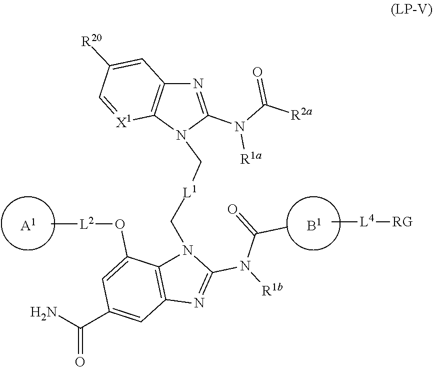
or a pharmaceutically acceptable salt or tautomer thereof,
wherein
Ring B¹ is an optionally substituted C₃₋₁₂ carbocycle or an optionally substituted 3- to 12-membered heterocycle, wherein said optionally substituted C₃₋₁₂ carbocycle and optionally substituted 3- to 12-membered heterocycle are optionally substituted with one or more R⁵²;
L⁴ is a bond or a linker; and
RG is a reactive linker group.

58. (canceled)

59. (canceled)

60. (canceled)

61. The linker-payload compound of claim 51, selected from the group consisting of: 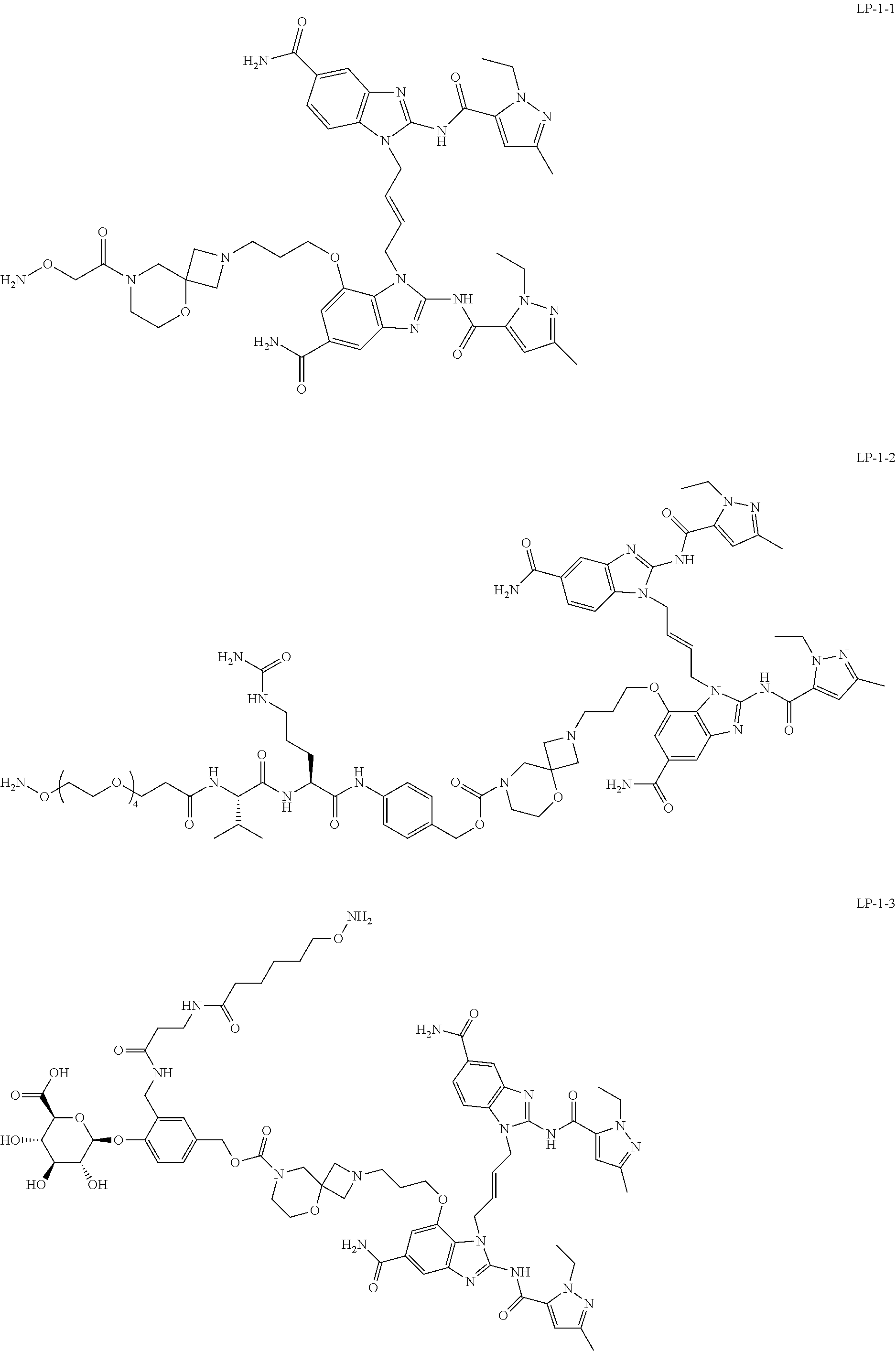 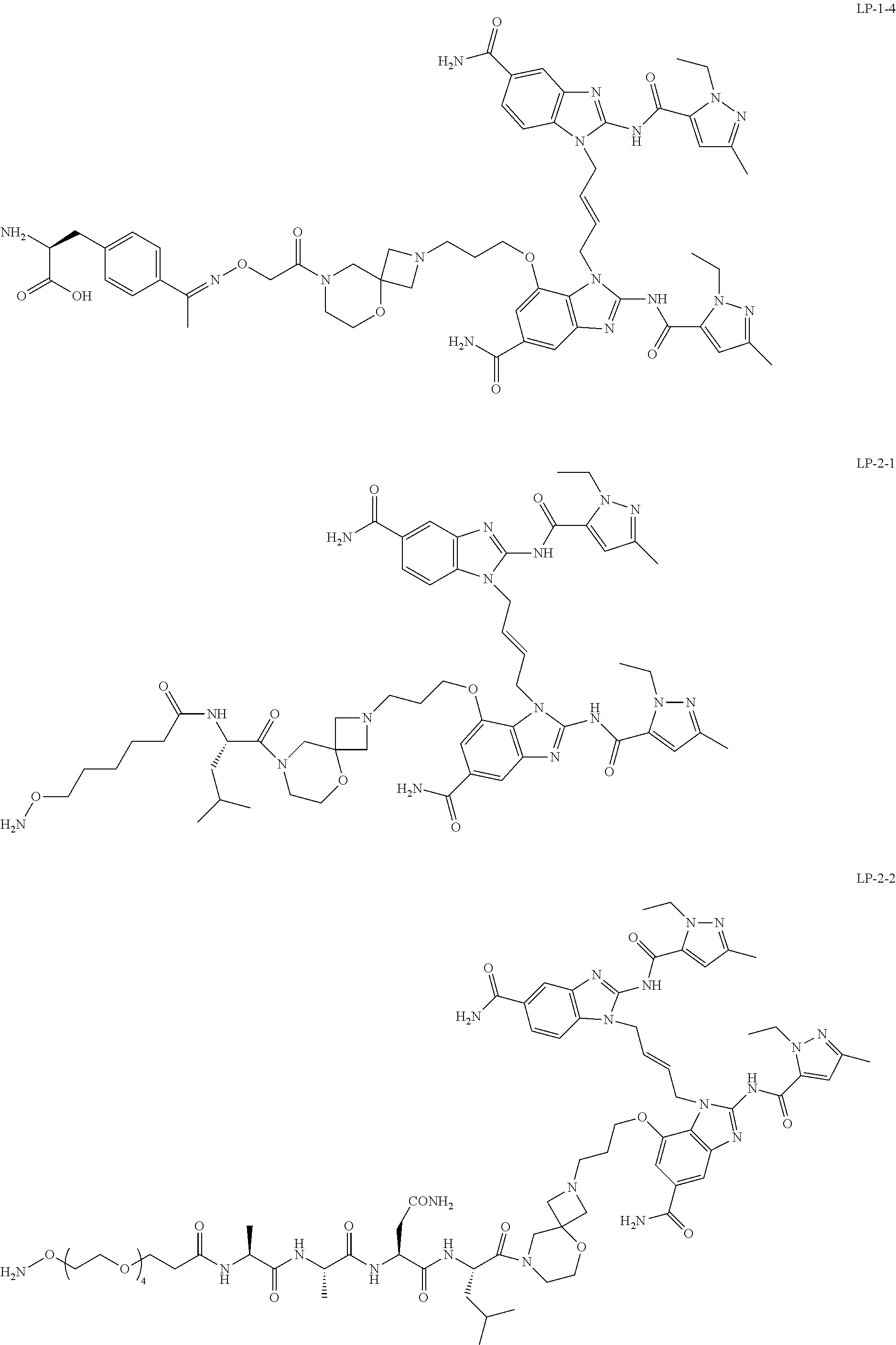 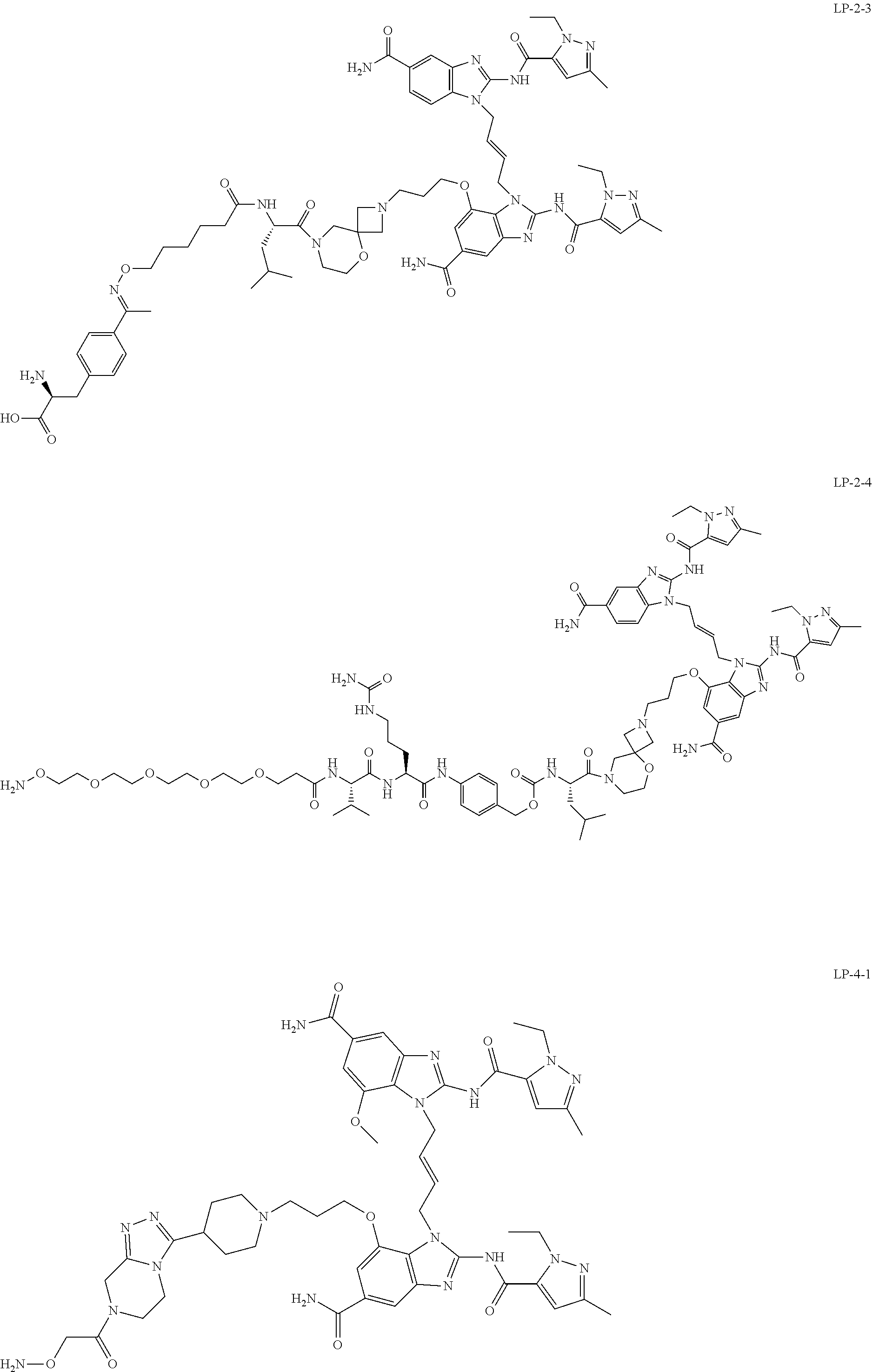 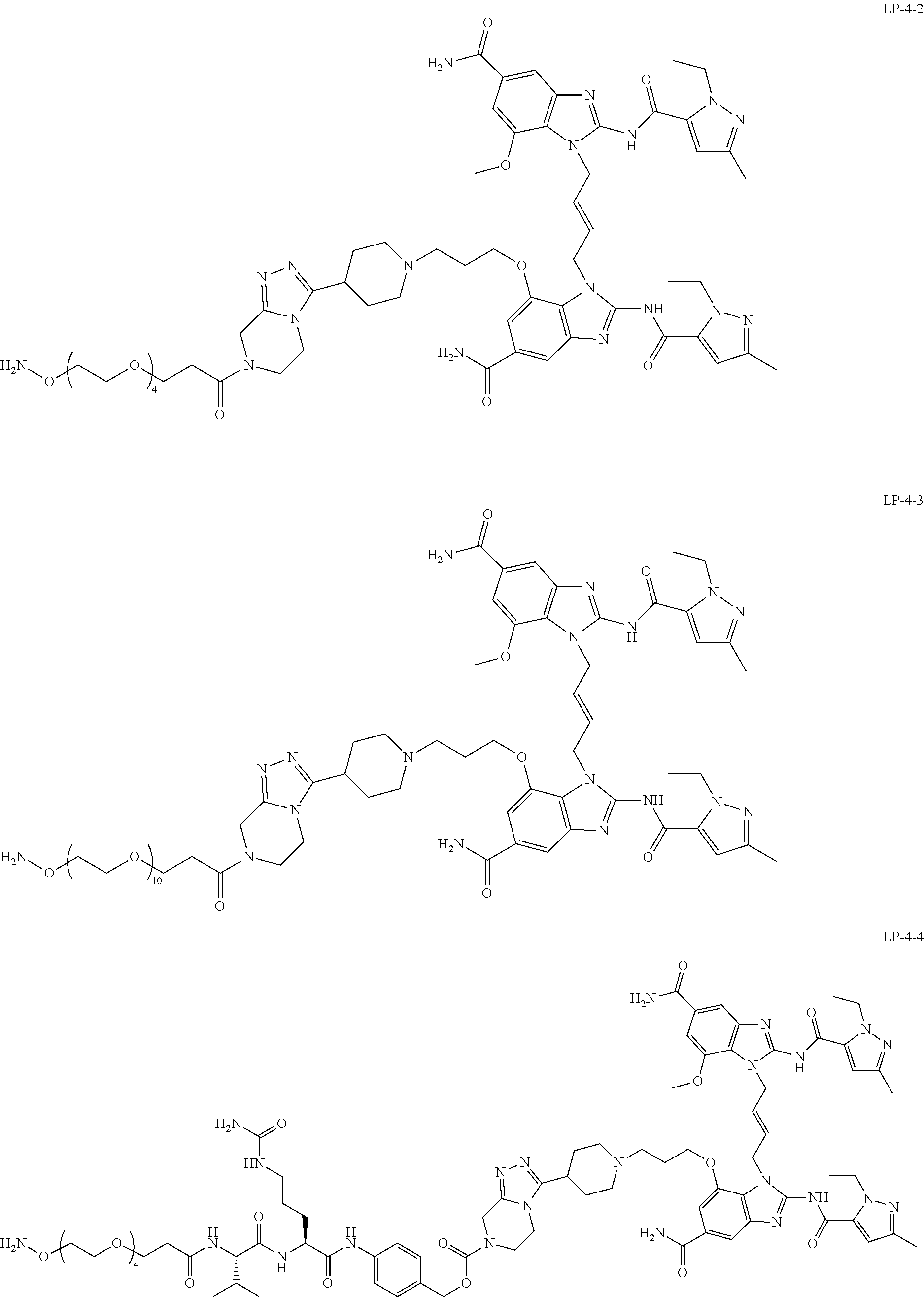 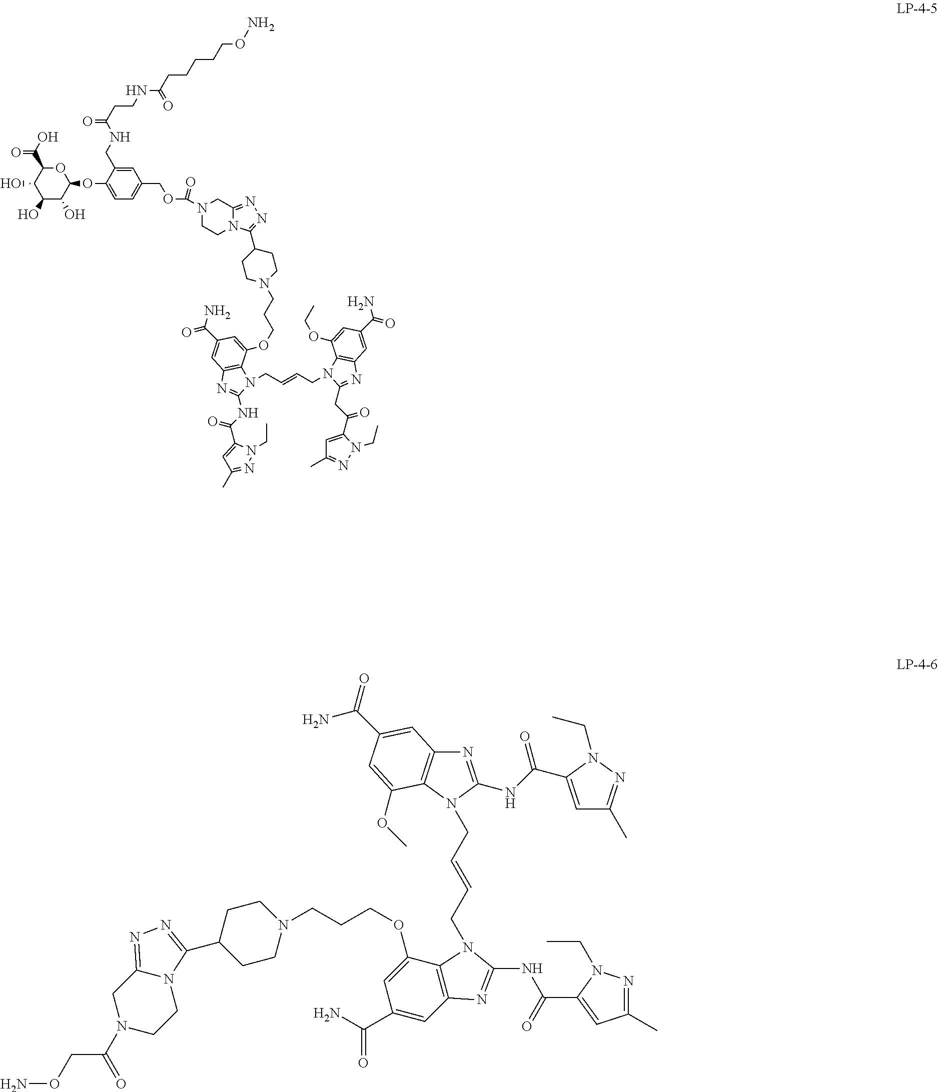 [SMILES: CCOc1cc(C(N)=O)cc2nc(CC(=O)c3cc(C)nn3CC)n(C/C=C/Cn3c(NC(=O)c4cc(C)nn4CC)nc4cc(C(N)=O)cc(OCCCN5CCC(c6nnc7n6CCN(C(=O)OCc6ccc(O[C@@H]8O[C@H](C(=O)O)[C@@H](O)[C@H](O)[C@H]8O)c(CNC(=O)CCNC(=O)CCCCCON)c6)C7)CC5)c43)c12 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21] 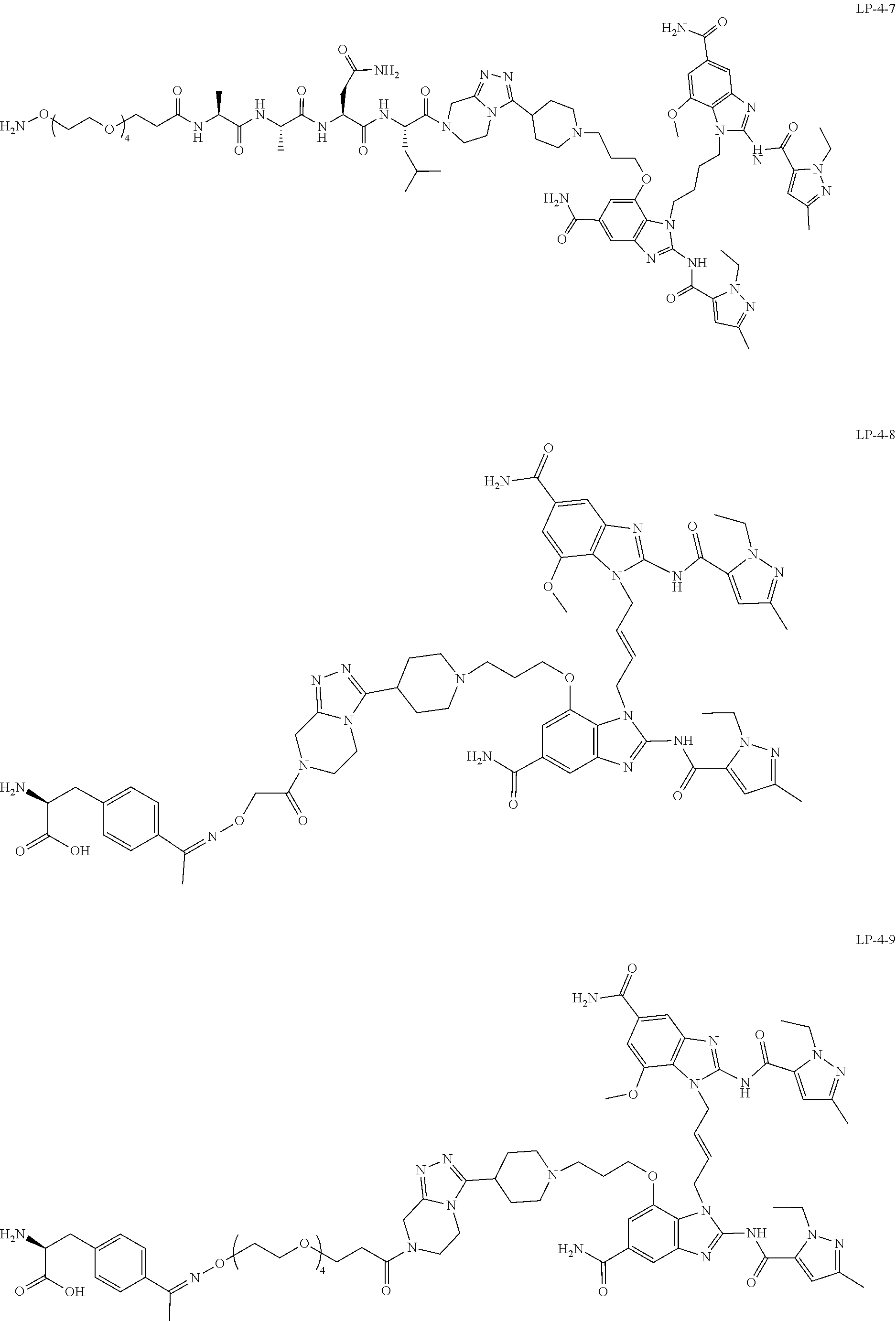 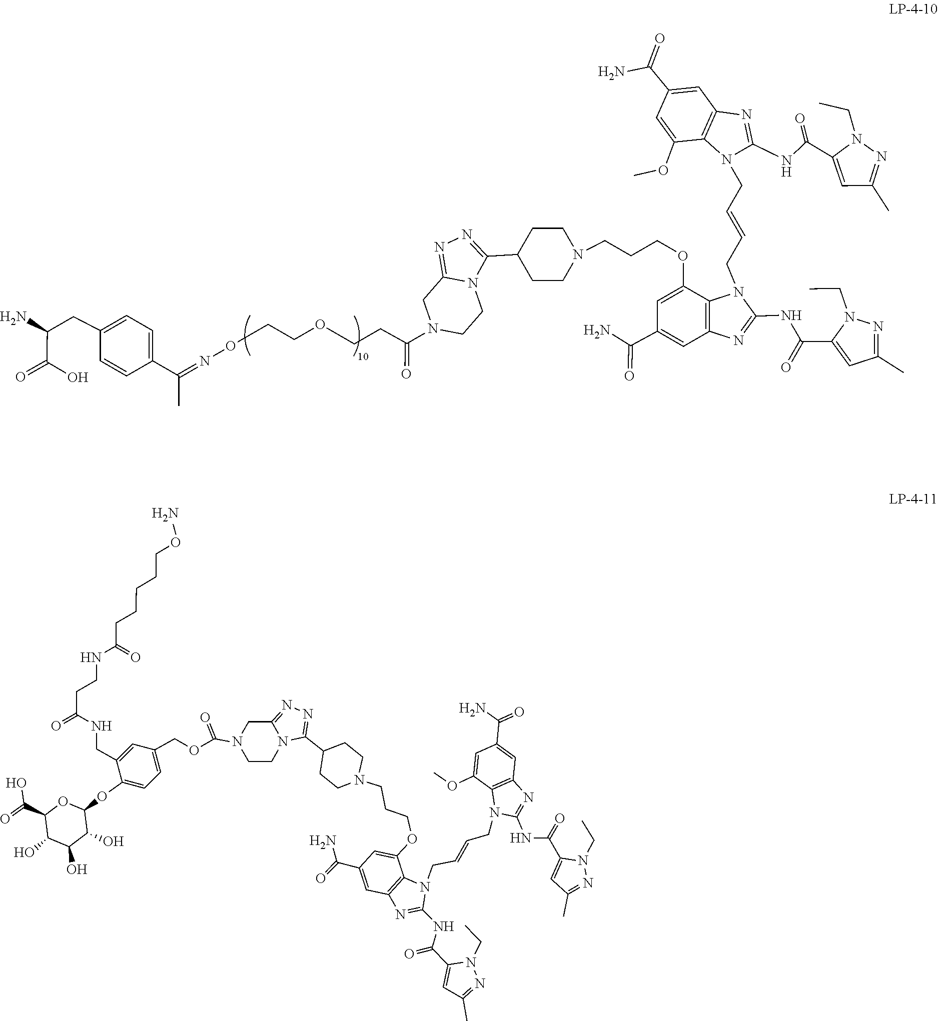 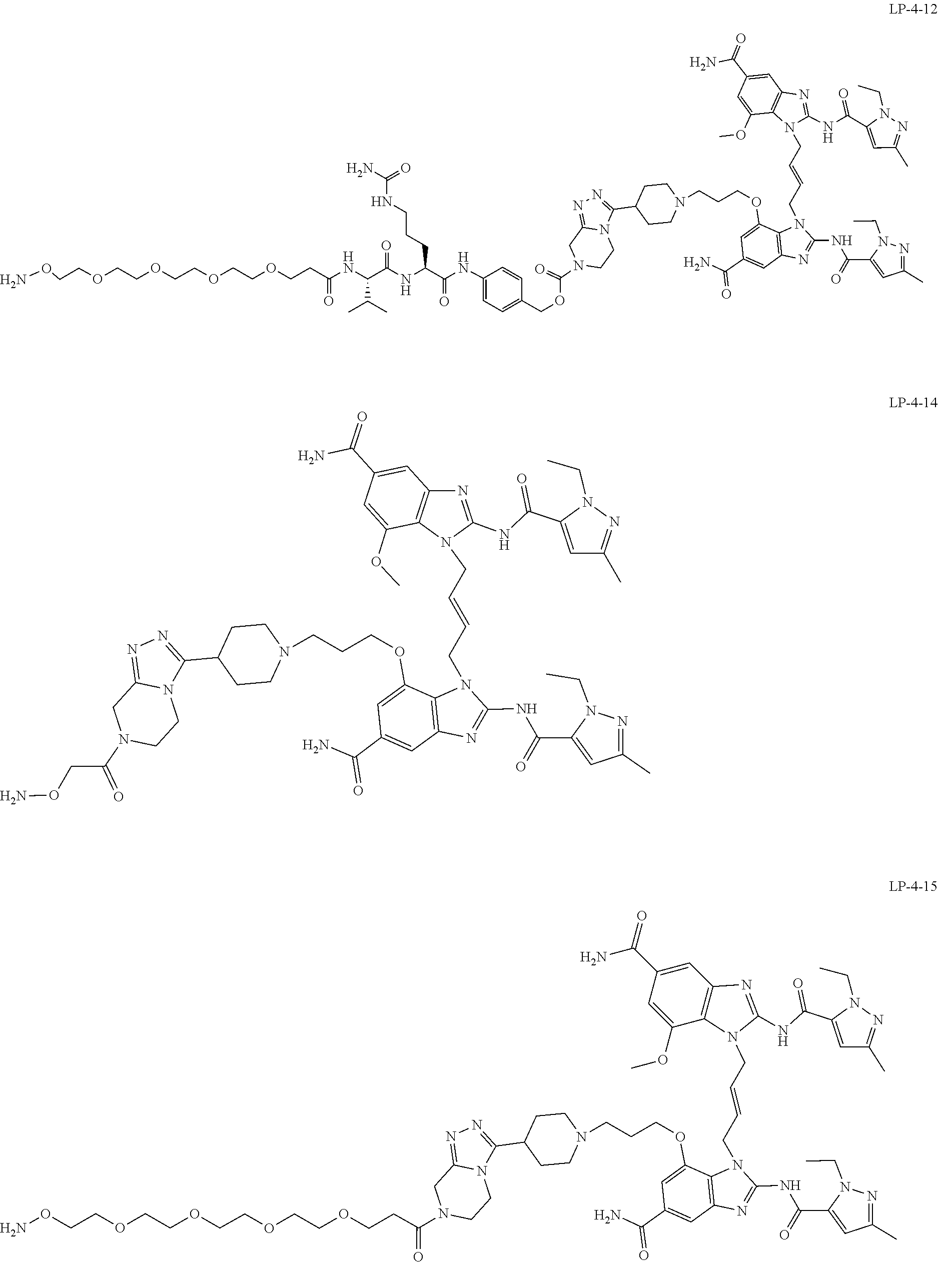 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(NC(=O)[C@H](CCCNC(N)=O)NC(=O)[C@@H](NC(=O)CCOCCOCCOCCOCCON)C(C)C)cc4)C5)CC3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCON)C5)CC3)c21] 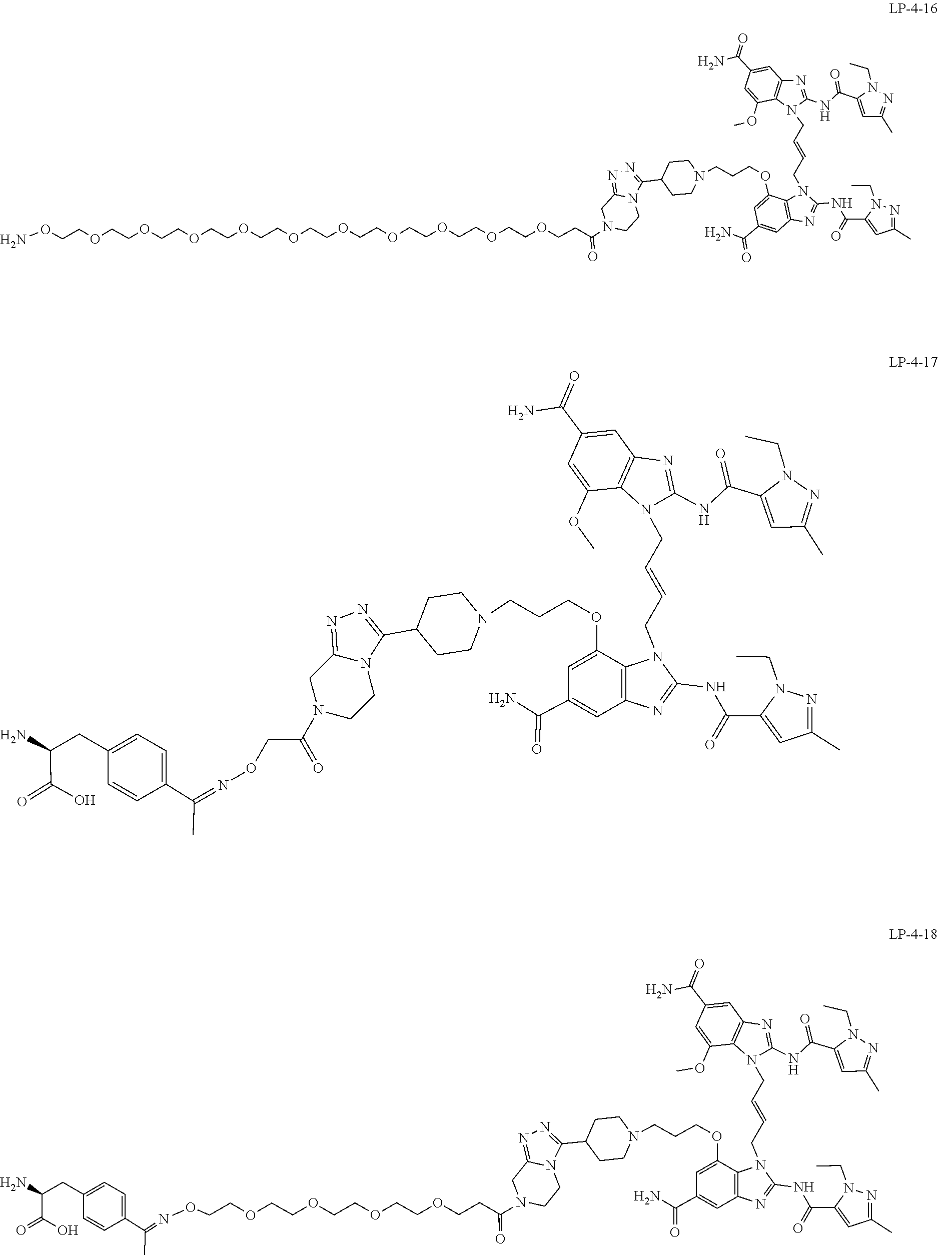 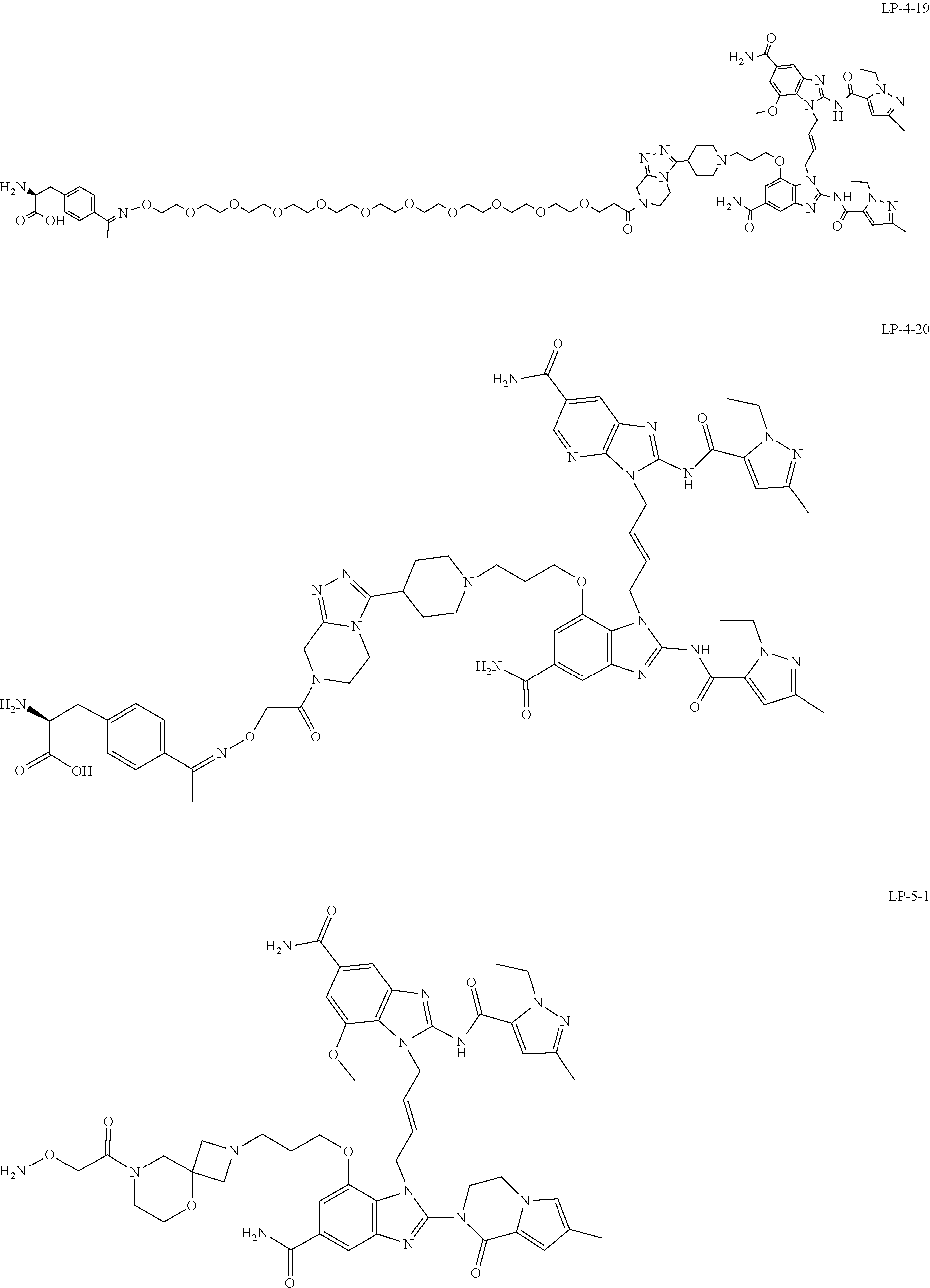 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCOCCOCCOCCOCCOCCOCCO/N=C(\C)c4ccc(C[C@H](N)C(=O)O)cc4)C5)CC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(C[C@H](N)C(=O)O)cc4)C5)CC3)c21 ; CCn1nc(C)cc1C(=O)Nc1nc2cc(C(N)=O)cc(OC)c2n1C/C=C/Cn1c(N2CCn3cc(C)cc3C2=O)nc2cc(C(N)=O)cc(OCCCN3CC4(C3)CN(C(=O)CON)CCO4)c21] 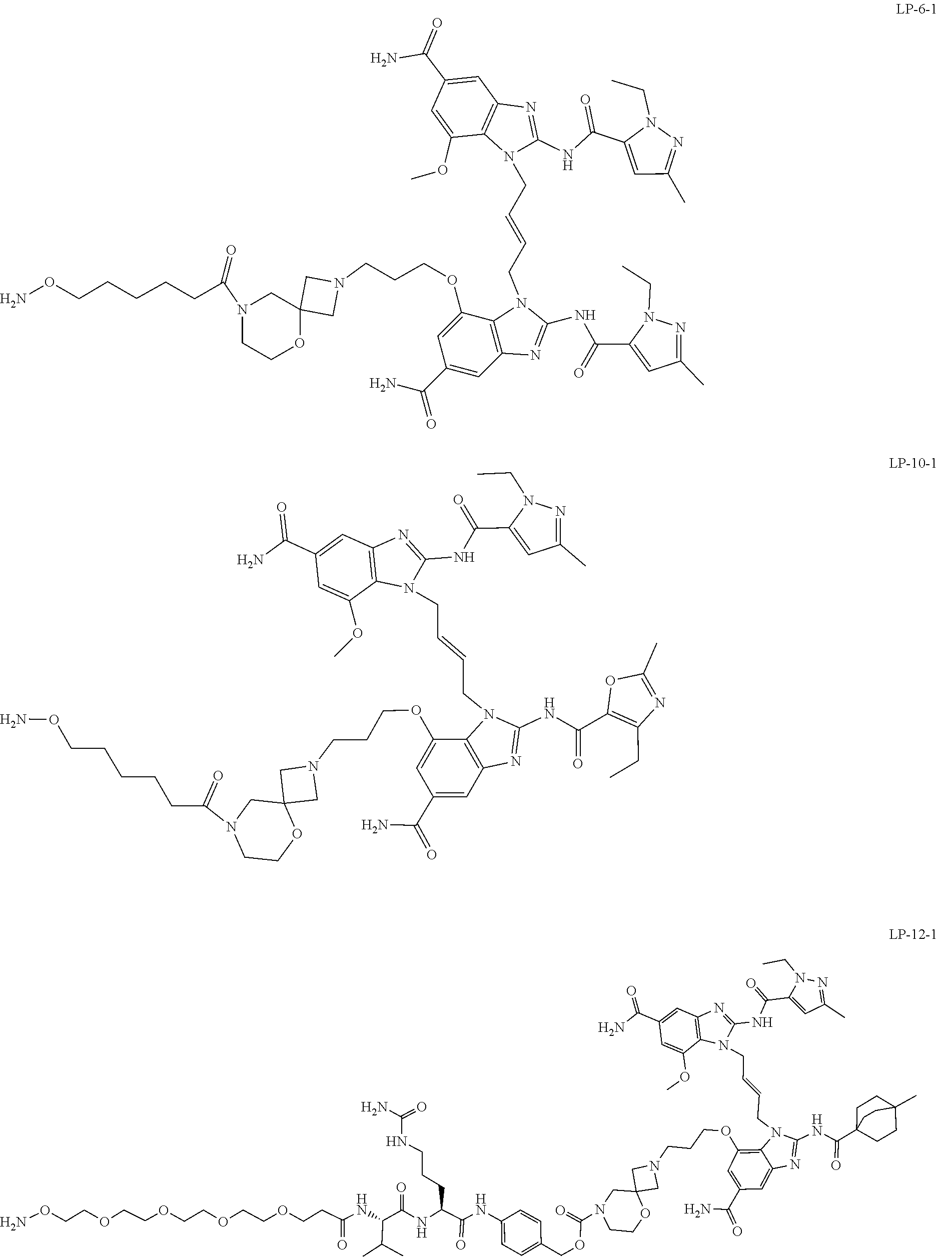 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(C3)CN(C(=O)CCCCCON)CCO4)c21 ; CCc1nc(C)oc1C(=O)Nc1nc2cc(C(N)=O)cc(OCCCN3CC4(C3)CN(C(=O)CCCCCON)CCO4)c2n1C/C=C/Cn1c(NC(=O)c2cc(C)nn2CC)nc2cc(C(N)=O)cc(OC)c21 ; CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(C3)CN(C(=O)OCc3ccc(NC(=O)[C@H](CCCNC(N)=O)NC(=O)[C@@H](NC(=O)CCOCCOCCOCCOCCON)C(C)C)cc3)CCO4)c21] 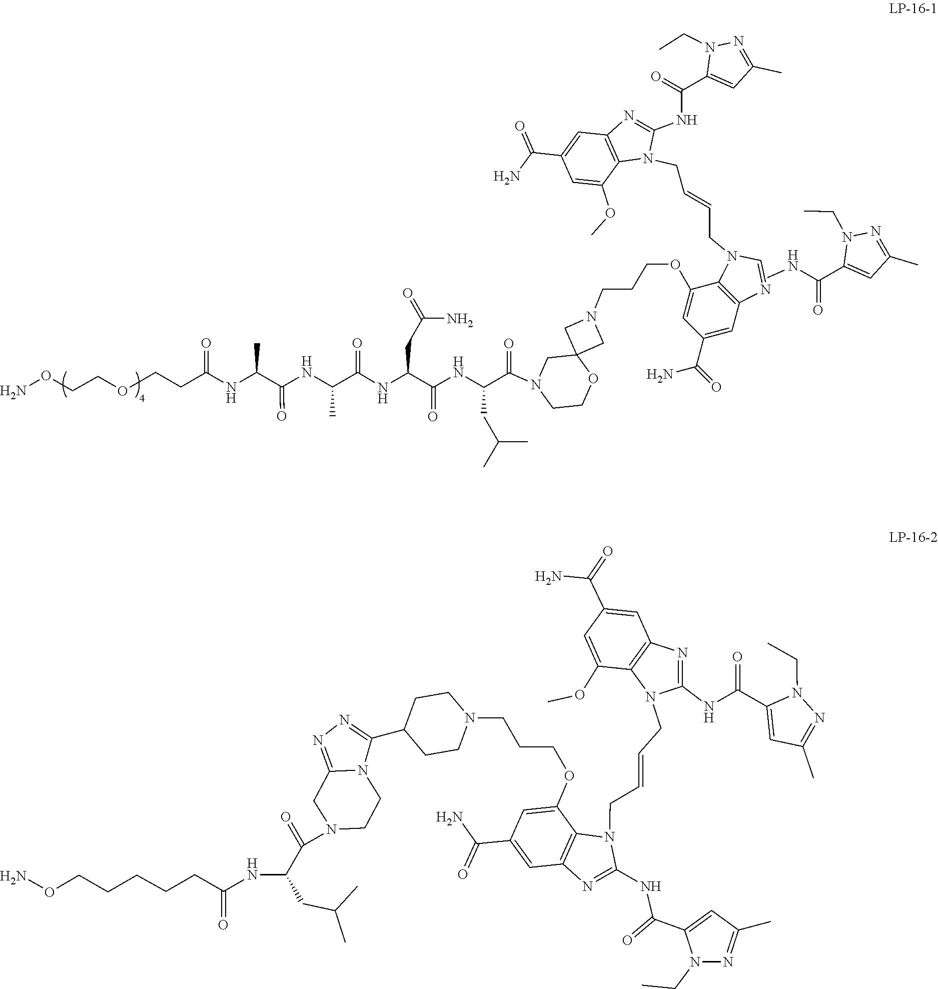 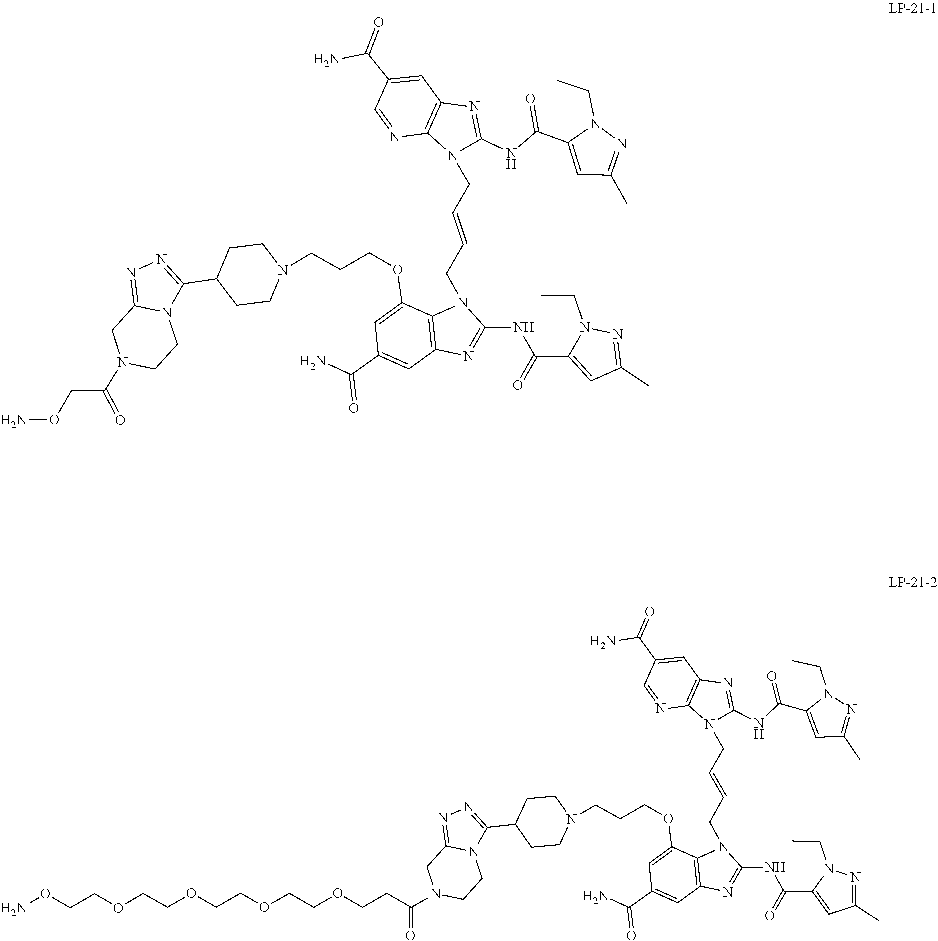 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CON)C5)CC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CCOCCOCCOCCOCCON)C5)CC3)c21] 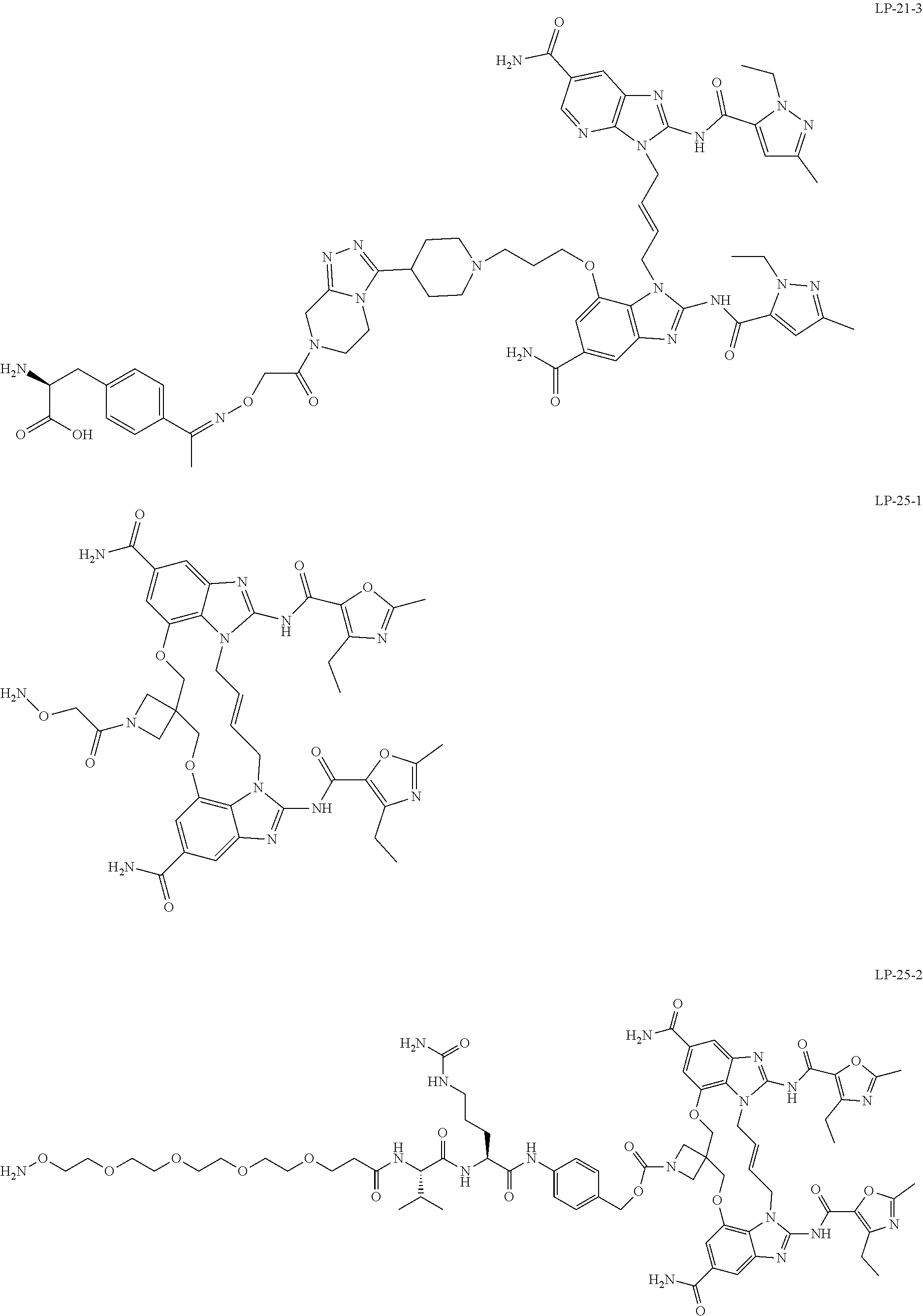 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CON=C(C)c4ccc(C[C@H](N)C(=O)O)cc4)C5)CC3)c21 ; CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CO3)CN(C(=O)CON)C1 ; CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CO3)CN(C(=O)OCc2ccc(NC(=O)[C@H](CCCNC(N)=O)NC(=O)[C@@H](NC(=O)CCOCCOCCOCCOCCON)C(C)C)cc2)C1] 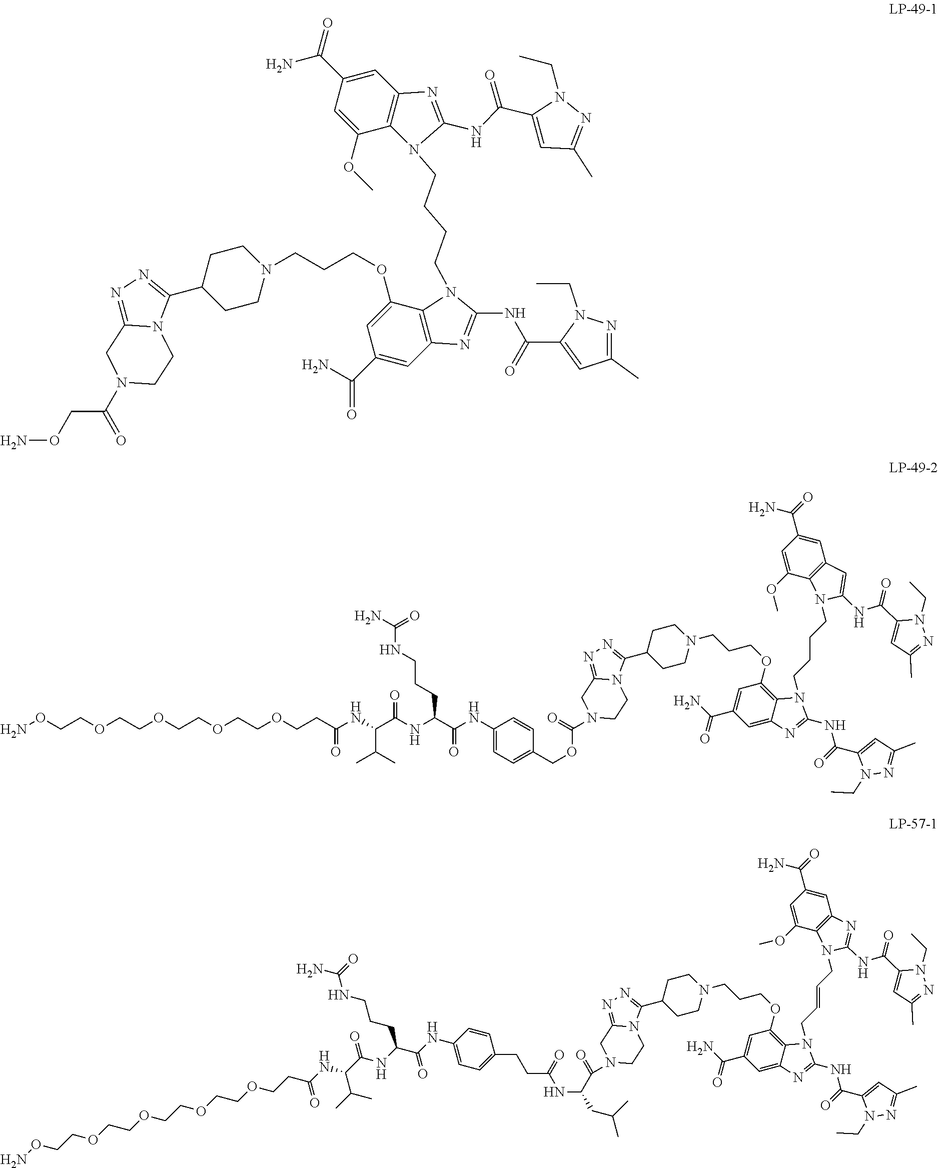 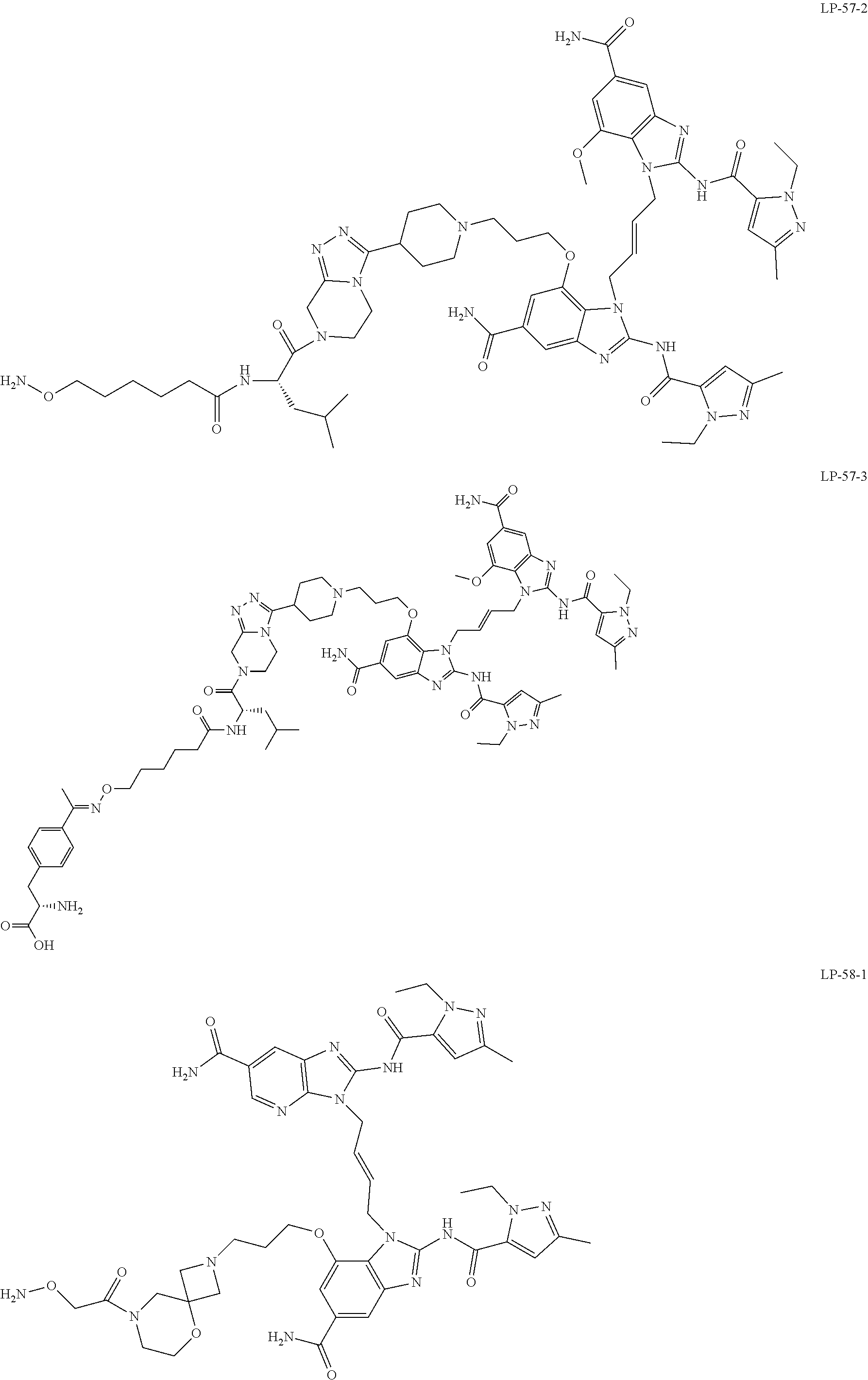 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)CCCCCON)C5)CC3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)CCCCCO/N=C(\C)c4ccc(C[C@H](N)C(=O)O)cc4)C5)CC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CC4(C3)CN(C(=O)CON)CCO4)c21] 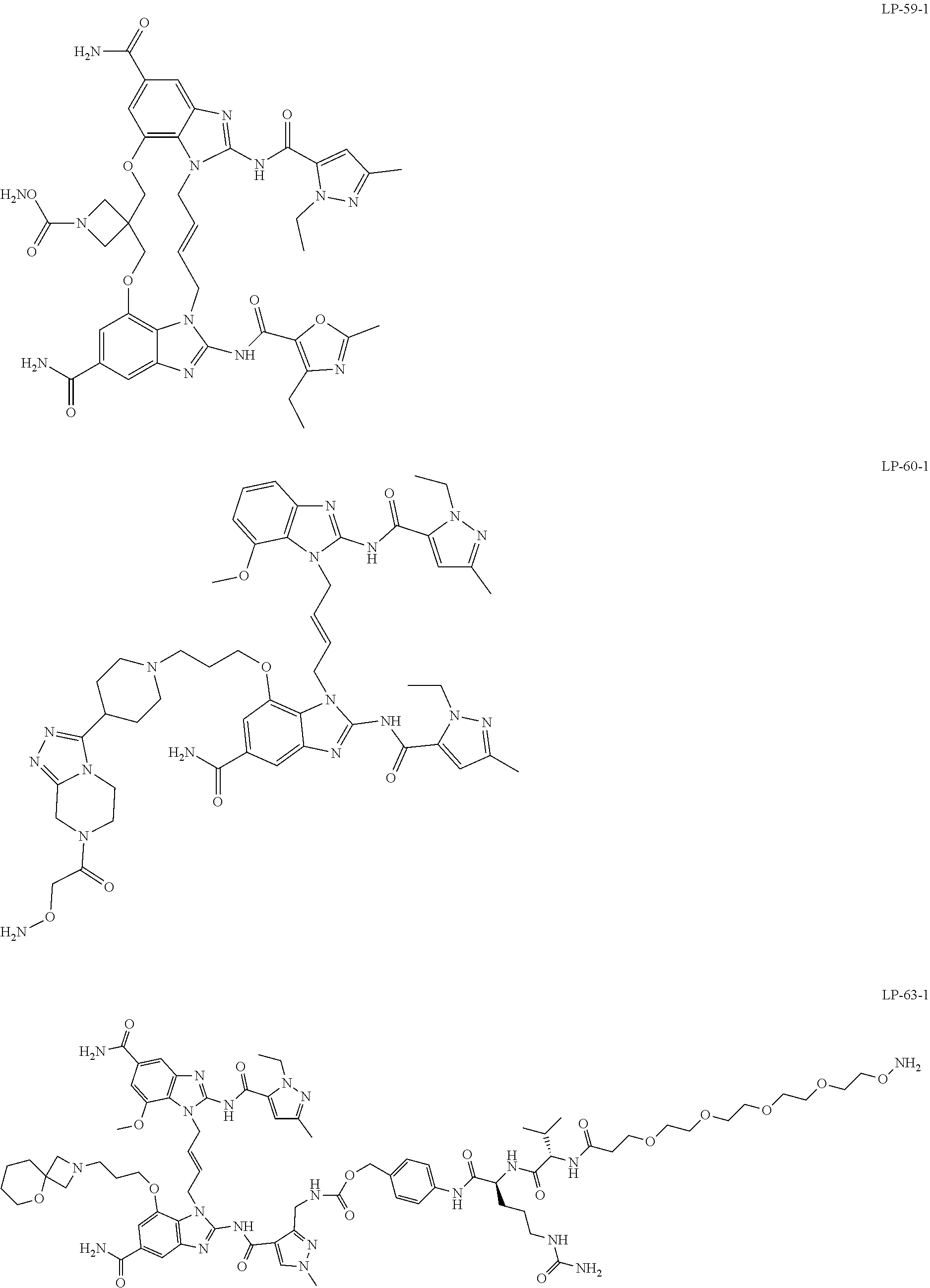 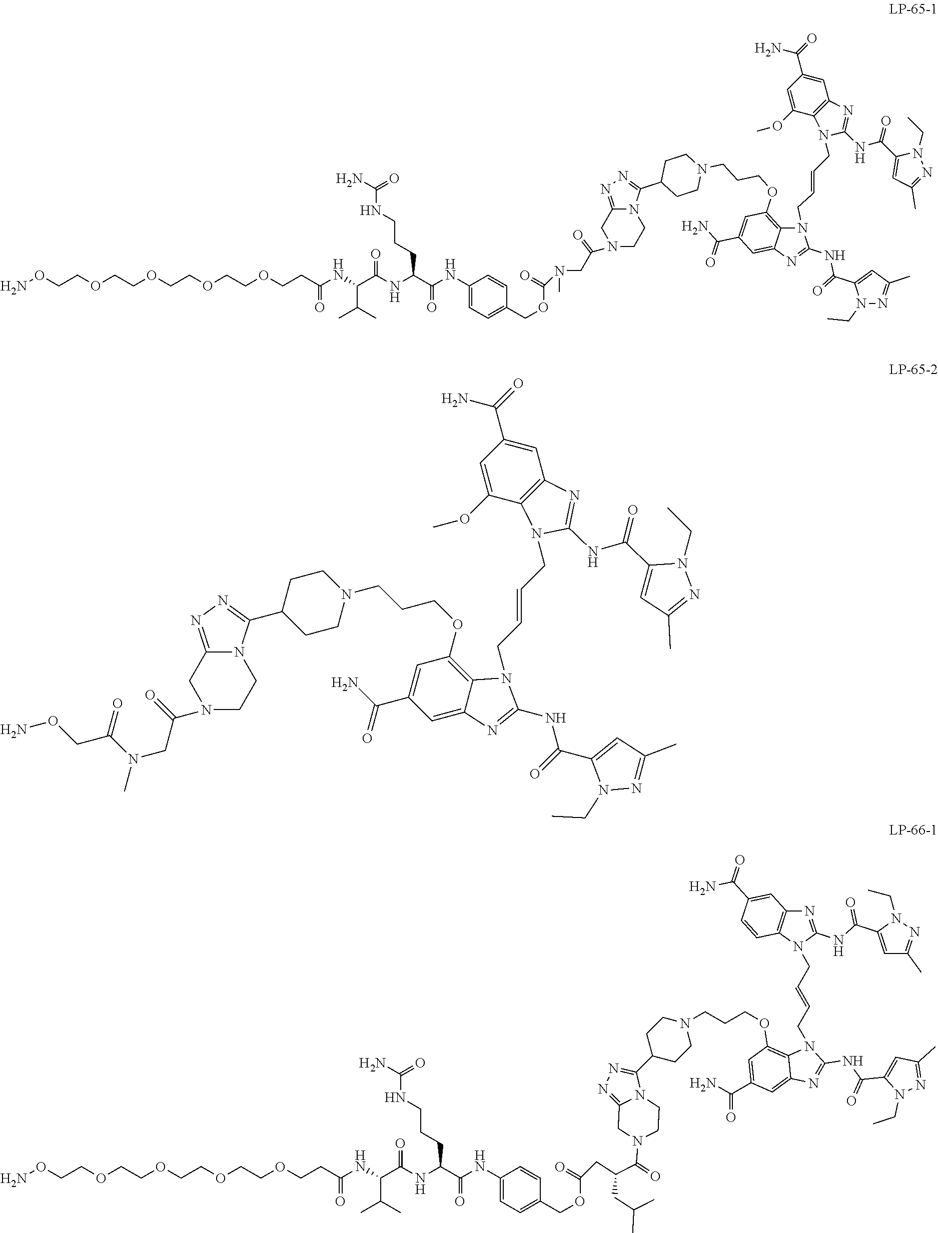
or a pharmaceutically acceptable salt or tautomer thereof.

62. The linker-payload compound of claim 51, selected from the group consisting of: 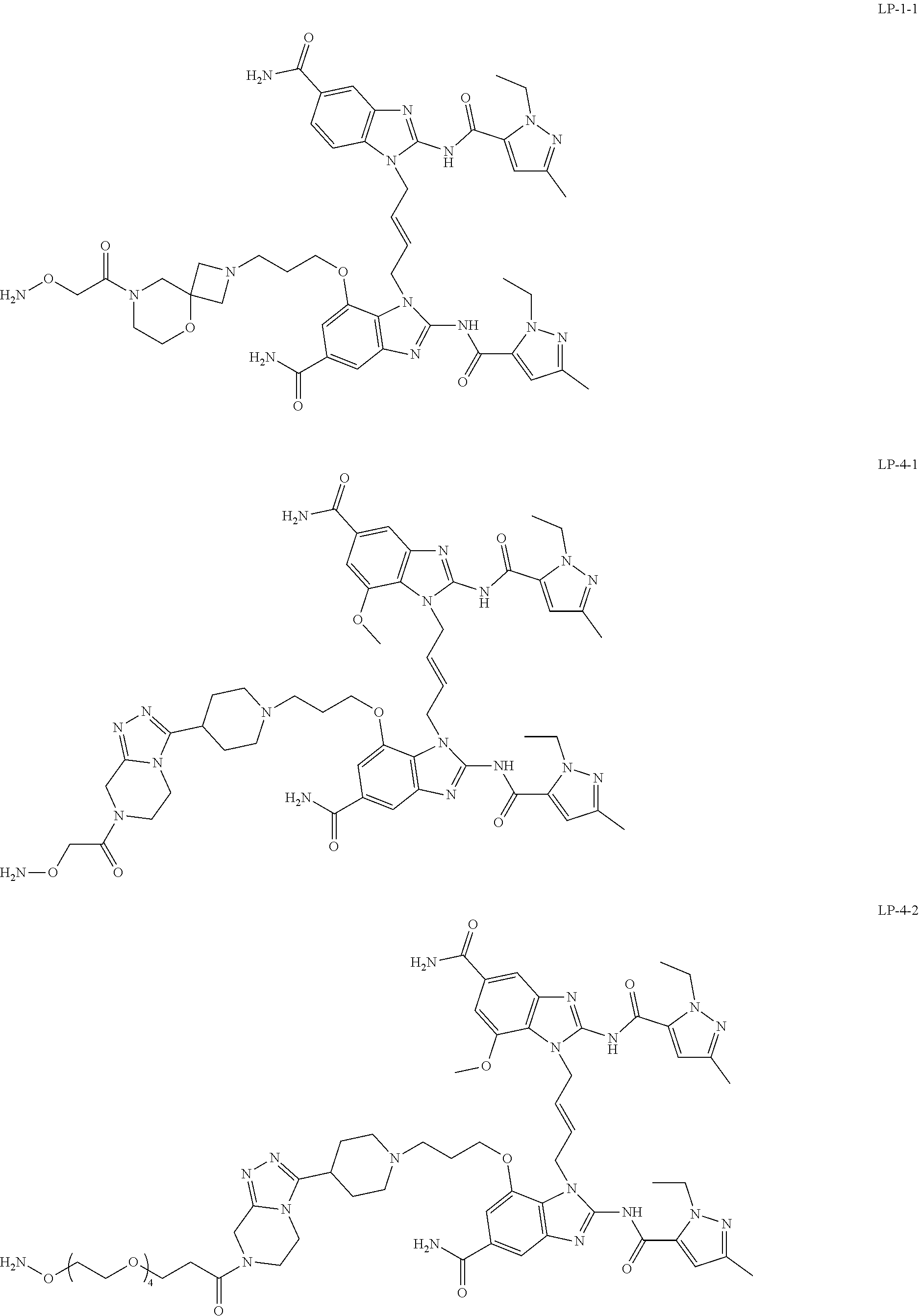 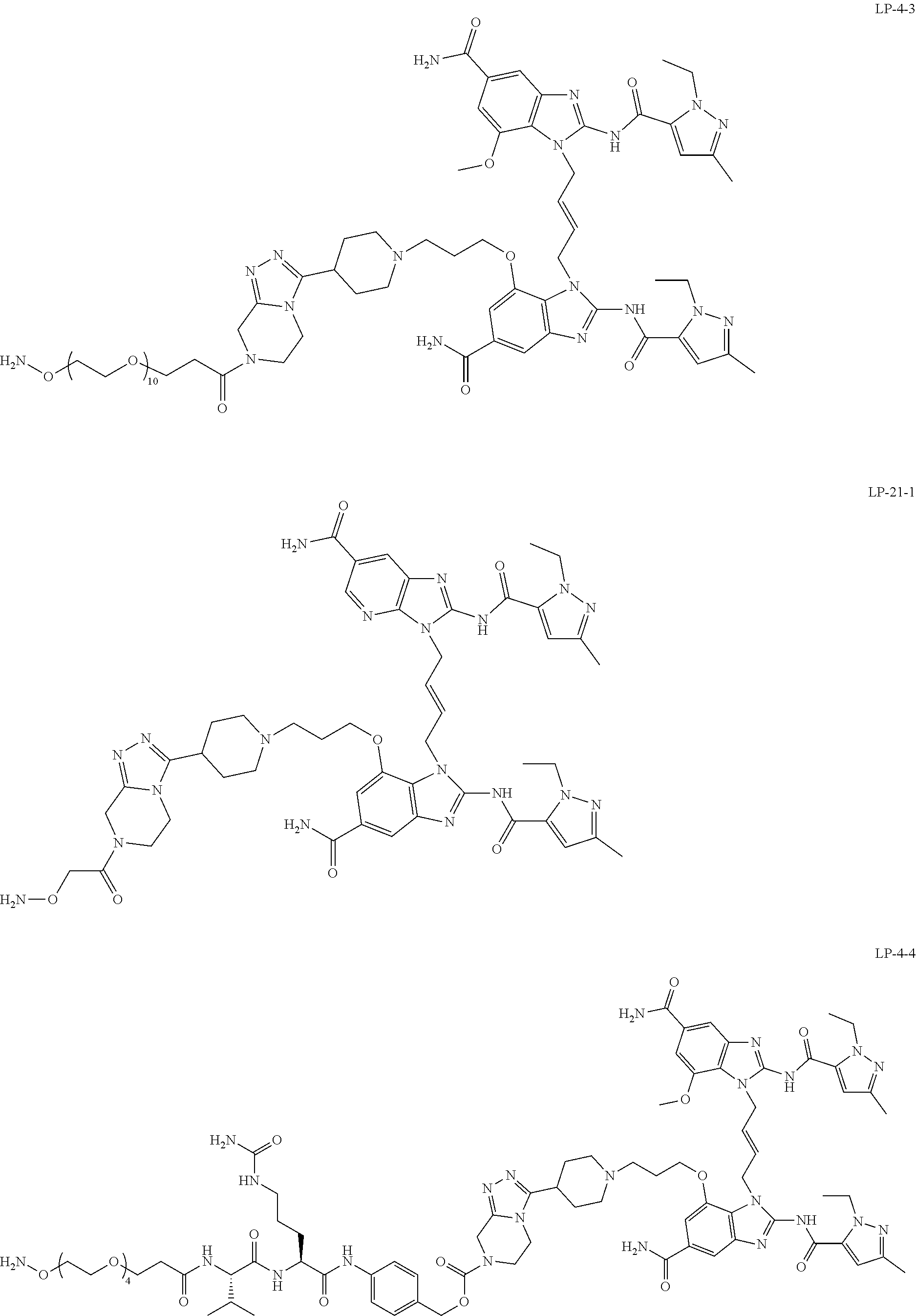
or a pharmaceutically acceptable salt or tautomer thereof.

63. A conjugate comprising the compound of claim 1 linked to COMP optionally via a linker, wherein COMP is a macromolecule and the linker comprises a protease cleavable linker, a pH sensitive linker, or a non-cleavable linker.

64. (canceled)

65. (canceled)

66. The conjugate of claim 63, according to the structure of Formula (CONJ-I) or (CONJ-IV): 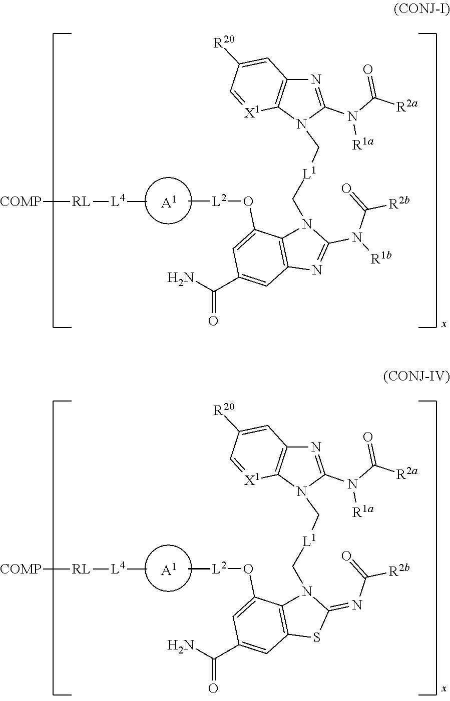
or pharmaceutically acceptable salt or tautomer thereof,
wherein
L⁴ is a bond or a linker;
RL is a reactive linker residue;
x is an integer between 1 and 30, inclusive; and
COMP is a macromolecule.

67. The conjugate of claim 63, according to the structure of Formula (CONJ-II): 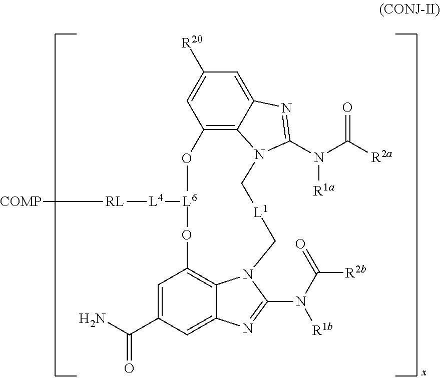
or a pharmaceutically acceptable salt or tautomer thereof;
wherein
L⁶ is a linker comprising —C₁₋₆ alkylene-, which is substituted with either 1) one R⁵⁷ or 2) R^{28a} and R^{28b}; and wherein the —C₁₋₆ alkylene- is optionally further substituted with one or more R⁵⁰; wherein
when L⁶ is substituted with R^{28a} and R^{28b}, R^{28a} and R^{28b} are joined together with the atoms to which they are attached to form a C₃₋₁₂ carbocycle or a 3- to 12-membered heterocycle, wherein said C₃₋₁₂ carbocycle and 3- to 12-membered heterocycle are attached to -L⁴-RL-COMP and further optionally substituted with one or more R⁵²;
R⁵⁷ is independently selected at each occurrence from —OR⁶³, —SR⁶³, —C(O)N(R⁶⁰)(R⁶³), —N(R⁶³)C(O)R⁶⁰, —N(R⁶⁰)C(O)R⁶³, —N(R⁶³)C(O)N(R⁶⁰)₂, —N(R⁶⁰)C(O)N(R⁶⁰)(R⁶³), —N(R⁶⁰)(R⁶³), —C(O)R⁶³, —C(O)OR⁶³, —OC(O)R⁶³, =N(R⁶³), C₃₋₁₂ carbocycle substituted with R⁶³, and 3- to 12-membered heterocycle substituted with R⁶³;
R⁶³ is selected from —C₁₋₁₀ alkylene- attached to -L⁴-RL-COMP, —C₂₋₁₀ alkenylene-attached to -L⁴-RL-COMP, —C₂₋₁₀ alkynylene- attached to -L⁴-RL-COMP, C₃₋₁₂ carbocyclene attached to -L⁴-RL-COMP, and 3- to 12-membered heterocyclene attached to -L⁴-RL-COMP; and
L⁴ is a bond or a linker;
RL is a reactive linker residue;
x is an integer between 1 and 30, inclusive; and
COMP is a macromolecule.

68. The conjugate of claim 63, according to the structure of Formula (CONJ-III): 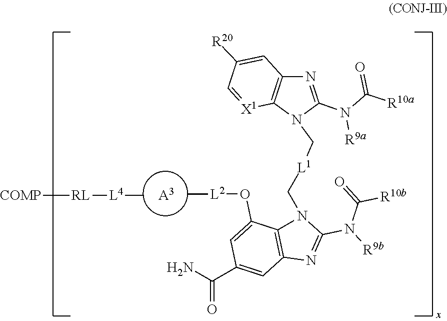
or a pharmaceutically acceptable salt or tautomer thereof;
wherein
L⁴ is a bond or a linker;
RL is a reactive linker residue;
x is an integer between 1 and 30, inclusive; and
COMP is a macromolecule.

69. The conjugate of claim 63, according to the structure of Formula (CONJ-V): 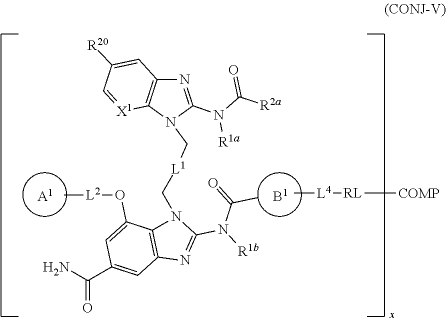
or a pharmaceutically acceptable salt or tautomer thereof,
wherein
Ring B¹ is an optionally substituted C₃₋₁₂ carbocycle or an optionally substituted 3- to 12-membered heterocycle, wherein said optionally substituted C₃₋₁₂ carbocycle and optionally substituted 3- to 12-membered heterocycle are optionally substituted with one or more R⁵²;
L⁴ is a bond or a linker;
RL is a reactive linker residue;
x is an integer between 1 and 30, inclusive; and
COMP is a macromolecule.

70. The conjugate of claim 66, wherein L⁴ is a linker that comprises a cathepsin cleavable linker, a legumain protease cleavable linker, a pH-sensitive linker, a b-glucuronidase cleavable linker or a non-cleavable linker.

71. The conjugate of claim 66, wherein R^{1a} and R^{1b} are both hydrogen.

72. The conjugate of claim 66, wherein R^{2a} and R^{2b} are independently an optionally substituted 3- to 12-membered heterocycle comprising at least one N atom.

73-78. (canceled)

79. The conjugate of claim 66, wherein L¹ is —CH═CH—, —CH₂—CH₂—or

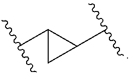 [SMILES: *C1CC1*]

80. (canceled)

81. (canceled)

82. The conjugate of claim 66, wherein X¹ is CH or CR₃ and R³ is OCH₃.

83-88. (canceled)

89. The conjugate of claim 66, wherein Ring A¹ is an optionally substituted 3- to 12-membered N-linked bridged, fused, or spirocyclic bicyclic heterocycle comprising at least one nitrogen atom, including the nitrogen bound to L², and at least one oxygen atom.

90. (canceled)

91. (canceled)

92. (canceled)

93. (canceled)

94. The conjugate of claim 66, wherein Ring A¹ is an N-linked 3- to 12-membered heterocycle substituted with R⁴.

95-106. (canceled)

107. The conjugate of claim 66, wherein L⁴ is of the formula: 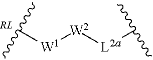
wherein
W¹ and W² are independently absent or a divalent attaching group;
L^{2a} is absent, a protease cleavable linker, or a pH-sensitive linker; 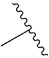
is the point of attachment to the rest of the compound; and 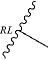
is a bond to RL.

108. (canceled)

109. (canceled)

110. The conjugate of claim 107, wherein W² is 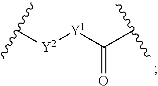
wherein Y¹ is absent or —C₁₋₁₀ alkylene-;
Y² is absent, a divalent water-soluble polymer, —NR¹⁴—C₁₋₁₀alkylene-, —NR¹⁴—C(O)—C₁₋₁₀alkylene-, or —O—C(O)—(C₁₋₁₀alkylene)-;
R¹⁴ is hydrogen or C₁₋₆ alkyl; and
wherein the C₁₋₁₀alkylene of Y¹ or Y² is optionally substituted with one, two, or three substituents selected from a halogen, alkyl, haloalkyl, hydroxyl, amino, alkylamino, and alkoxy; and wherein the carbonyl is attached to L^{2a}; and
wherein 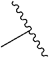
is the point of attachment to the rest of the compound.

111-120. (canceled)

121. The conjugate of claim 66, wherein RL is selected from the group consisting of 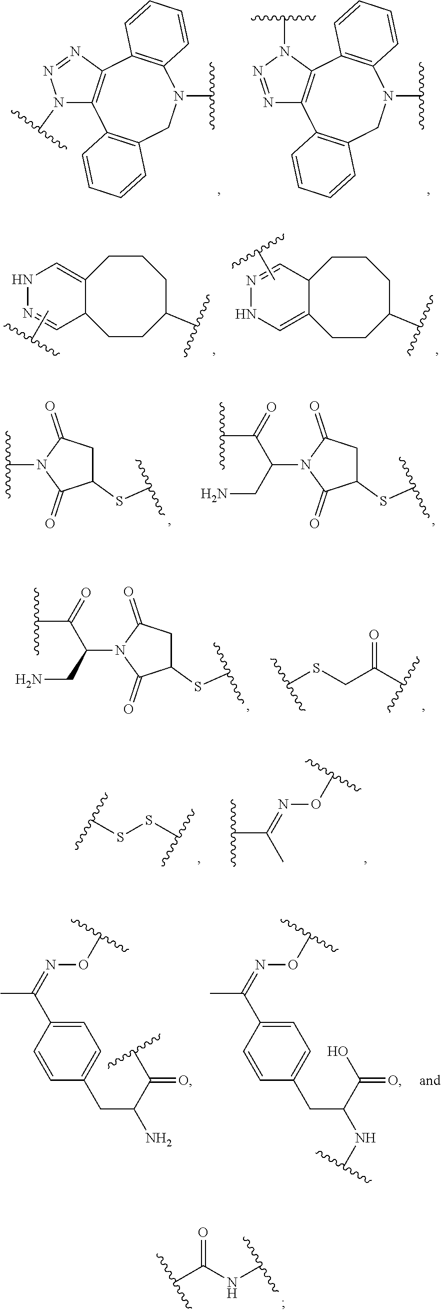
wherein 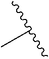
each is a of attachment to the rest of the compound.

122. (canceled)

123. (canceled)

124. (canceled)

125. (canceled)

126. (canceled)

127. The conjugate of claim 63, wherein the conjugate is selected from the group consisting of: 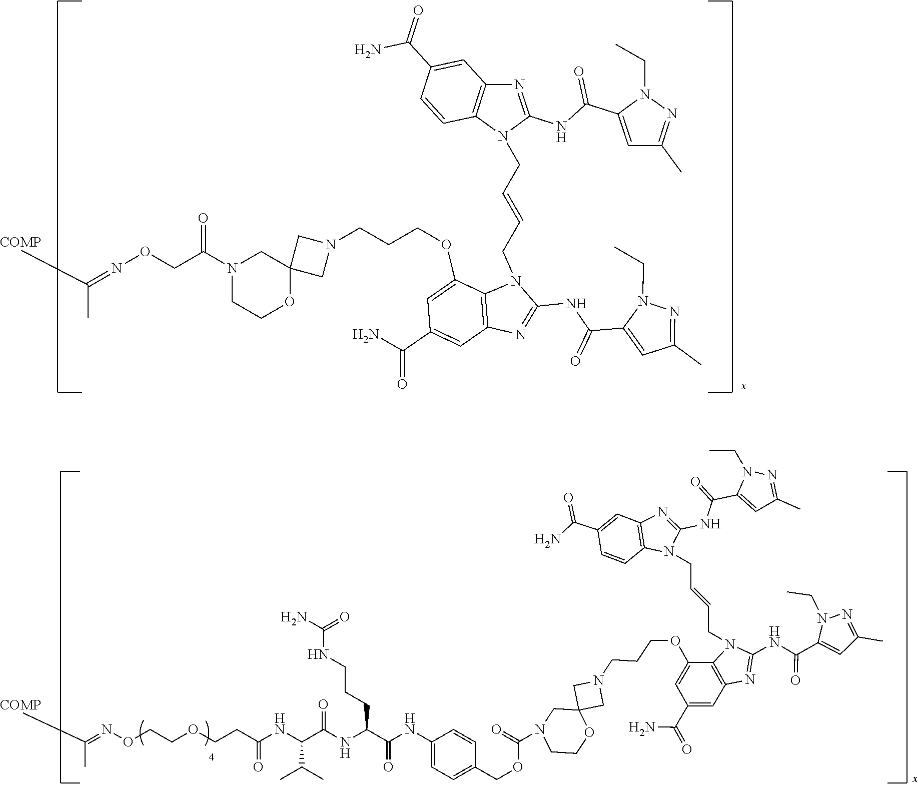 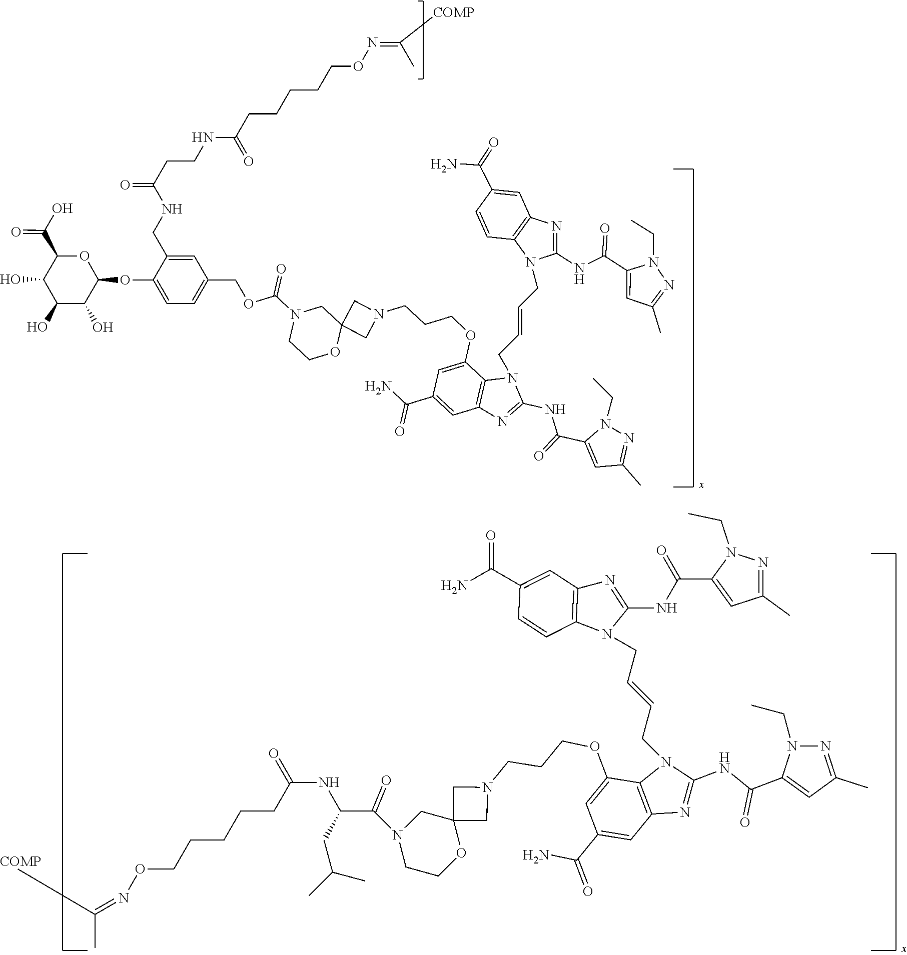 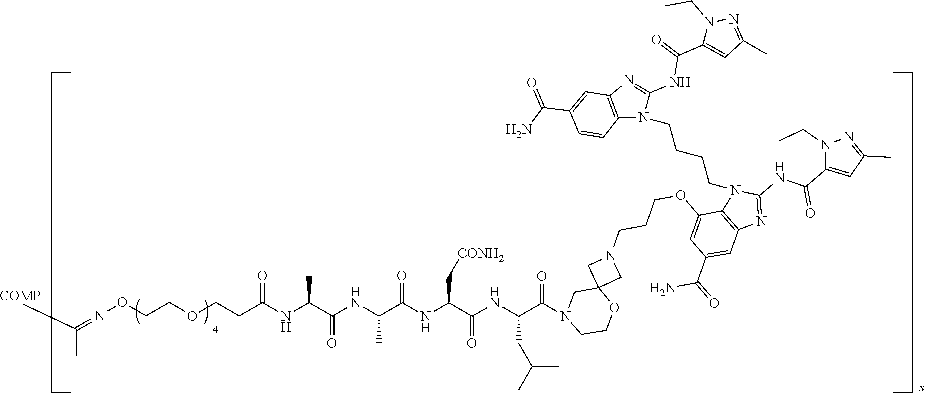 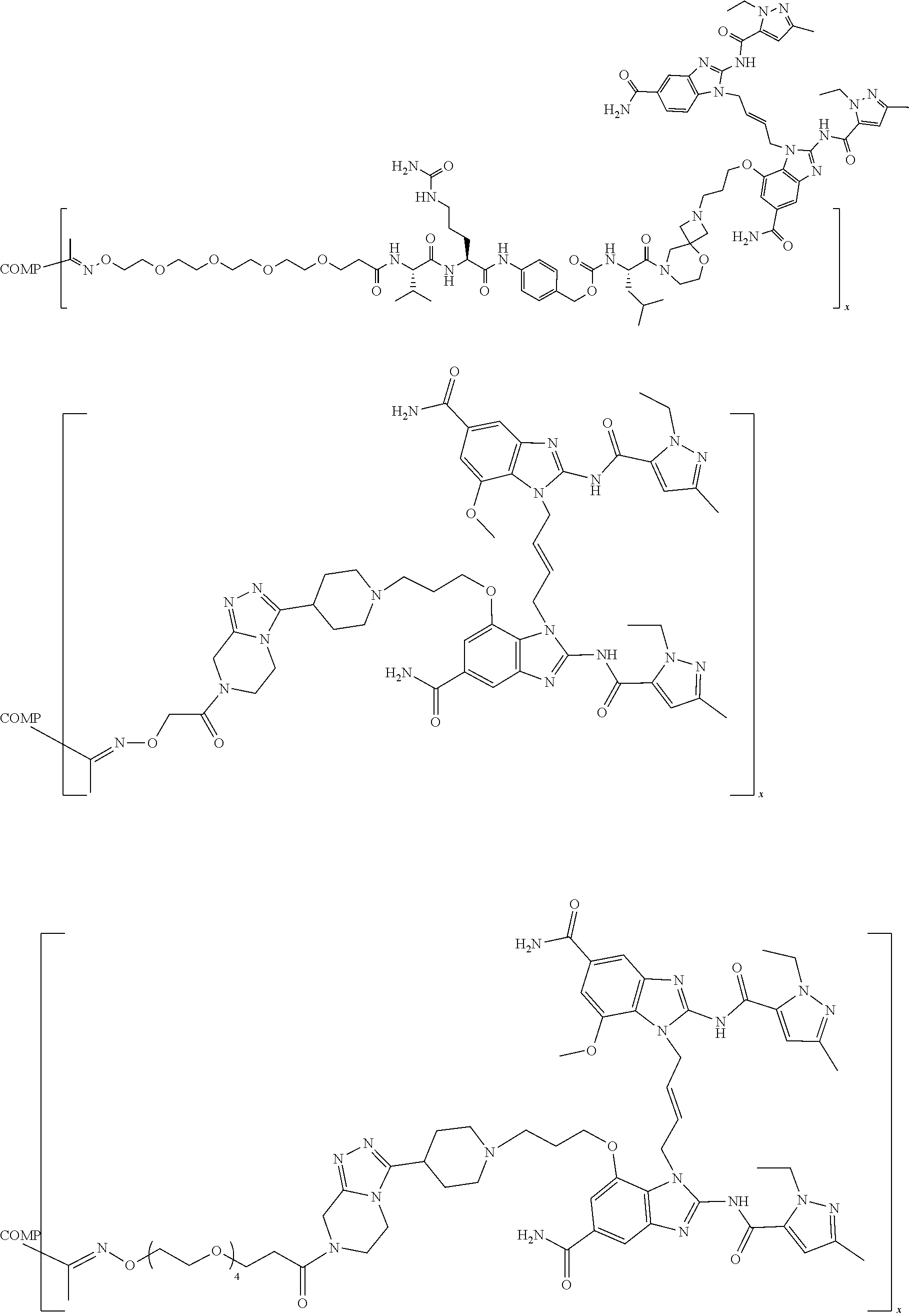 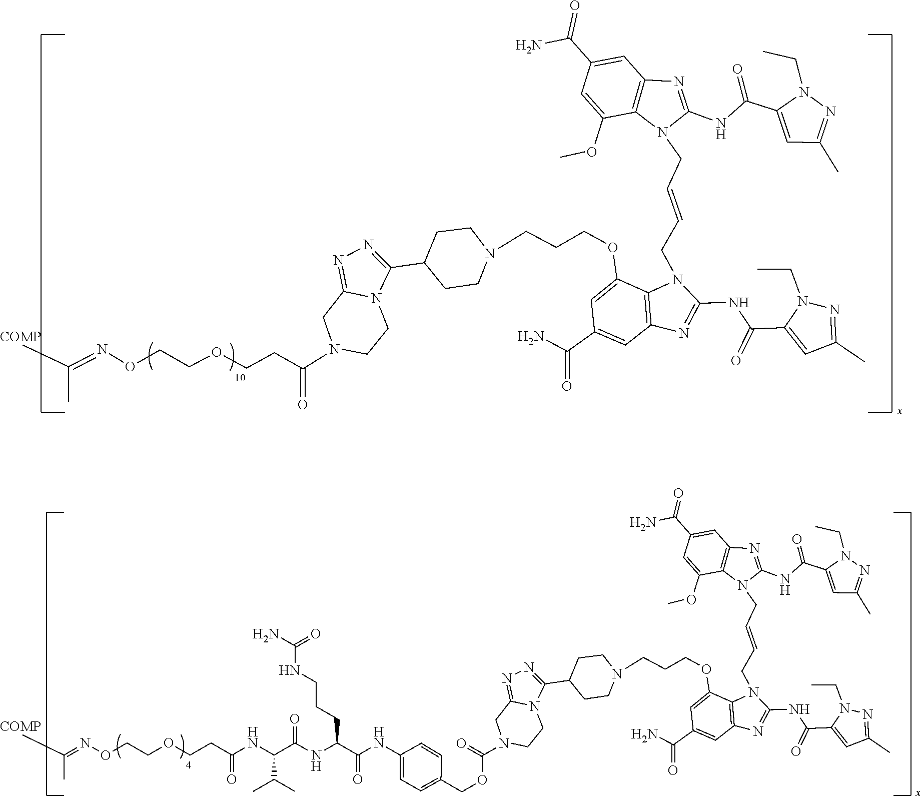 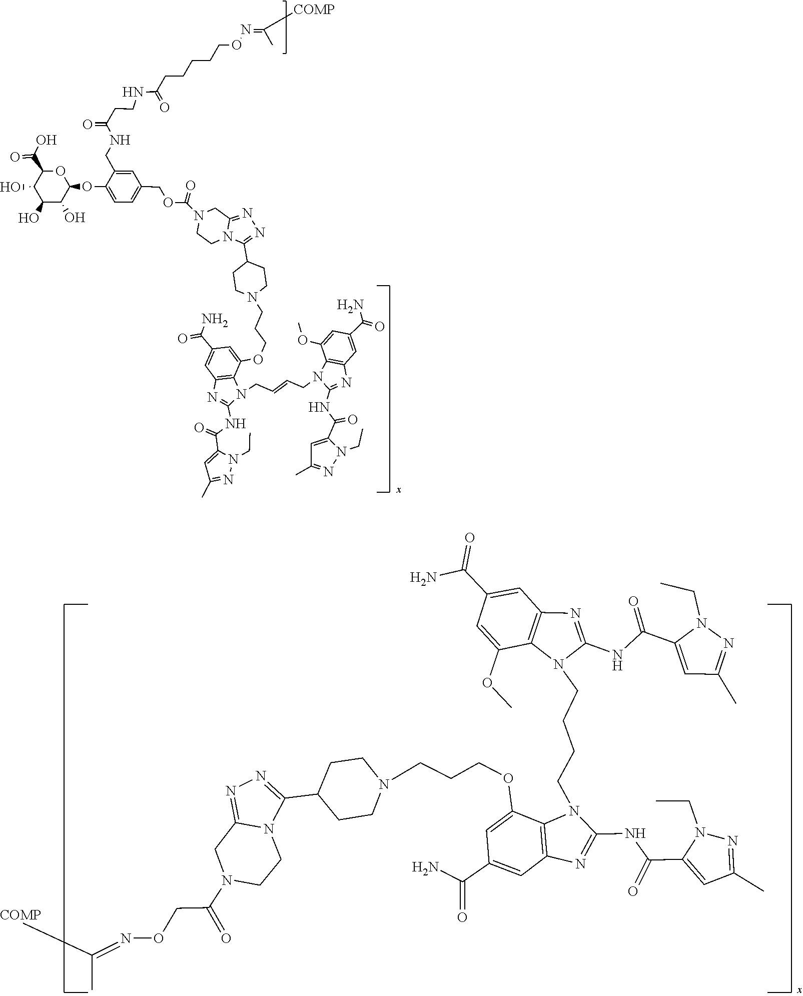 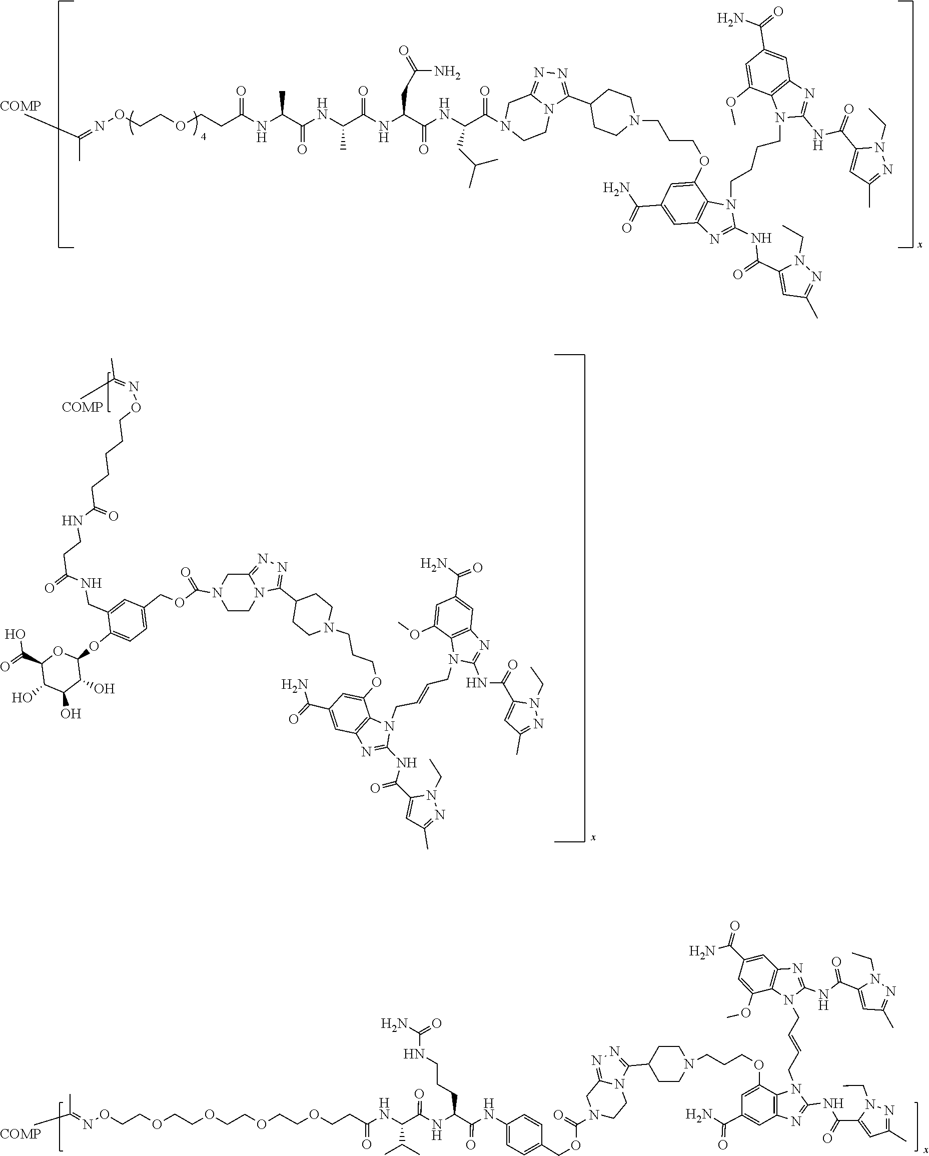 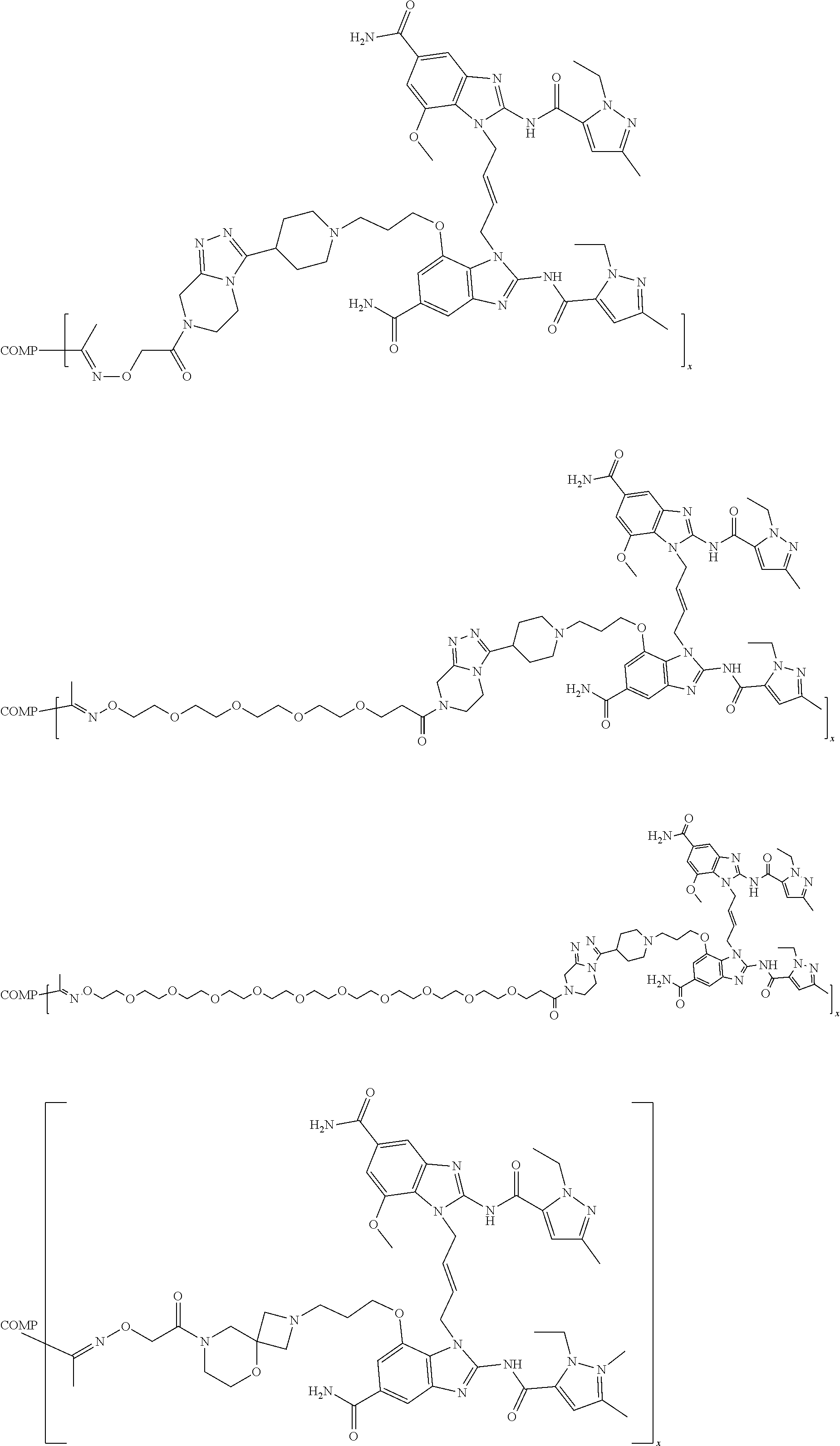 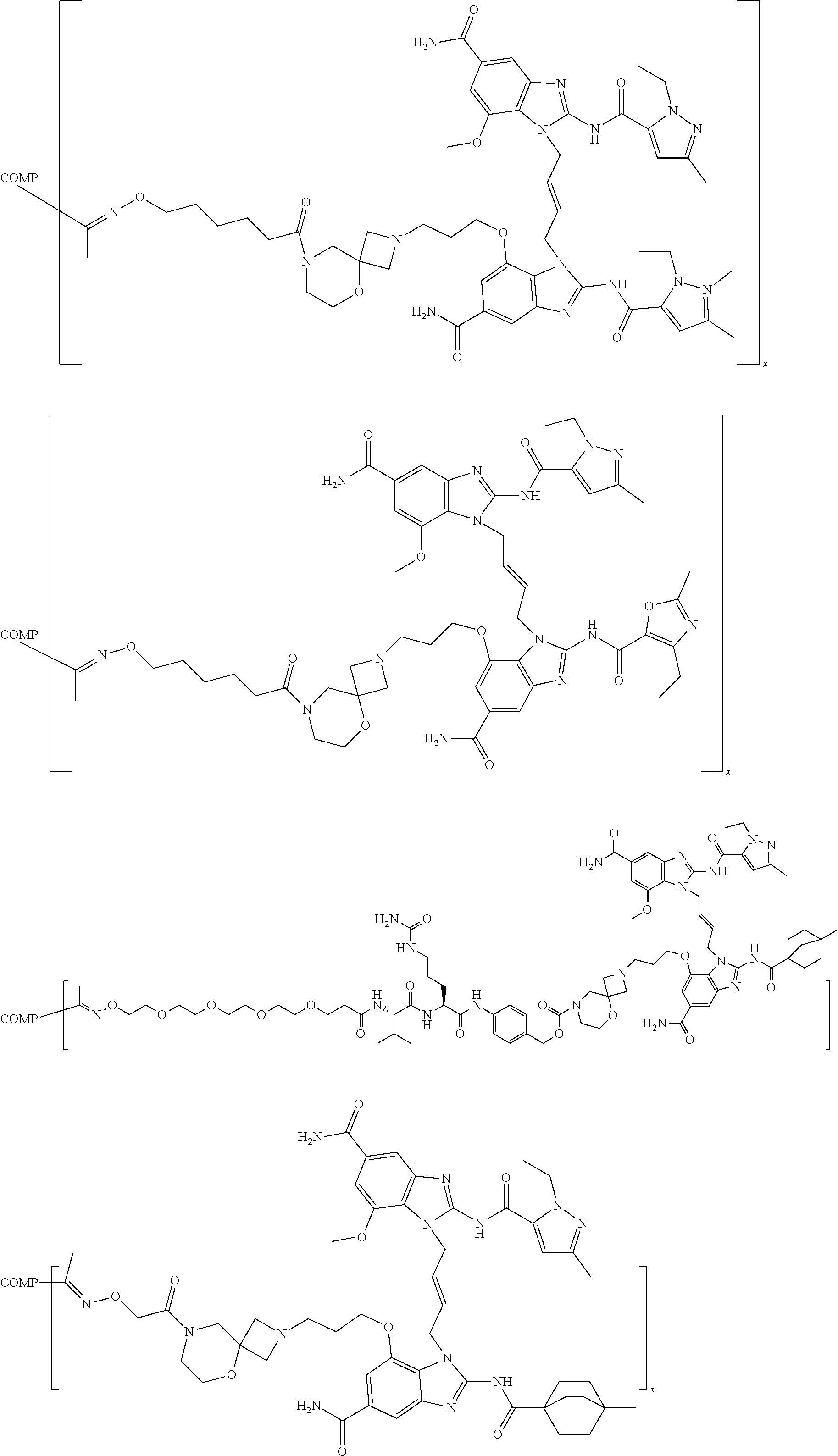 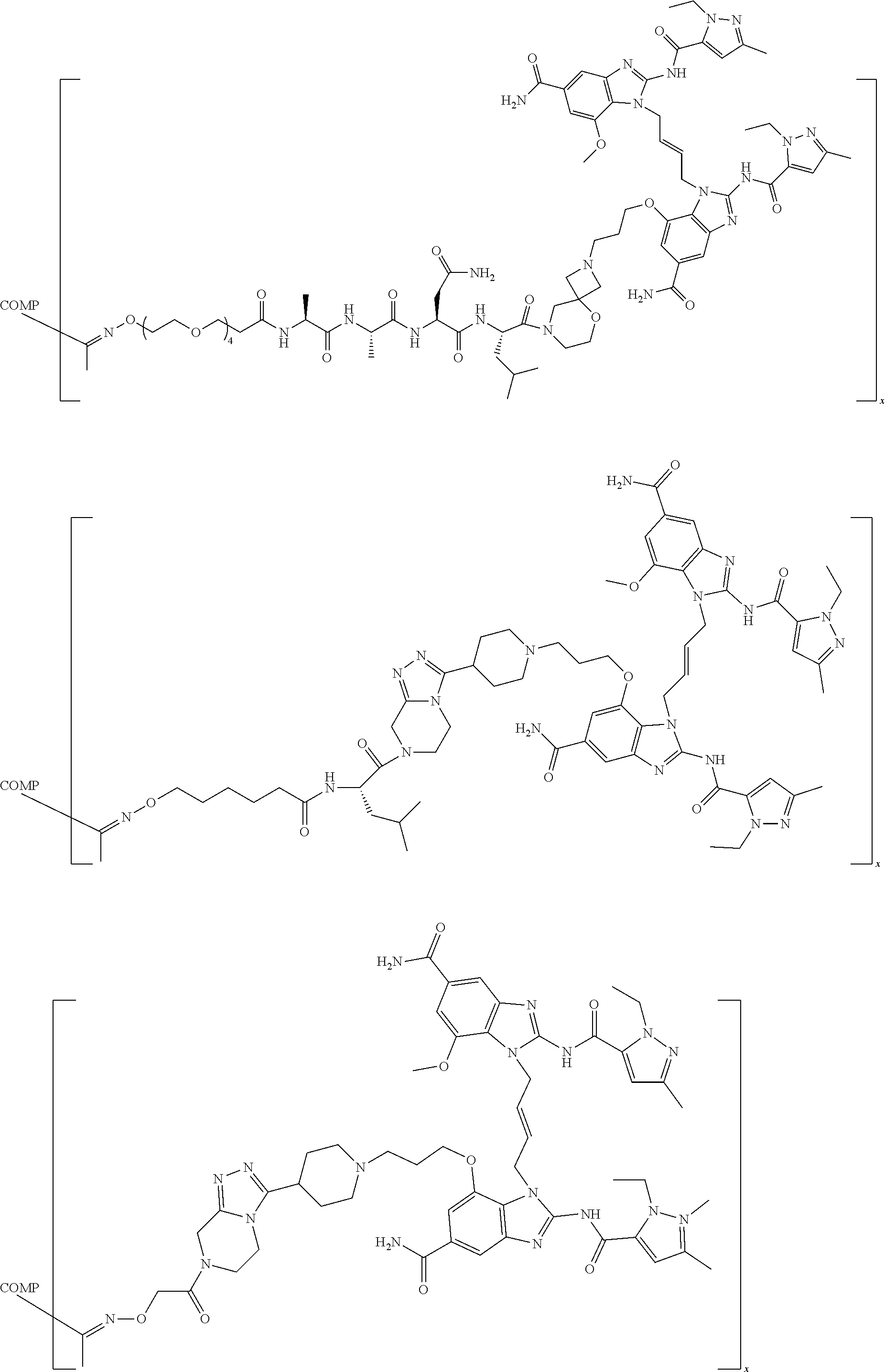 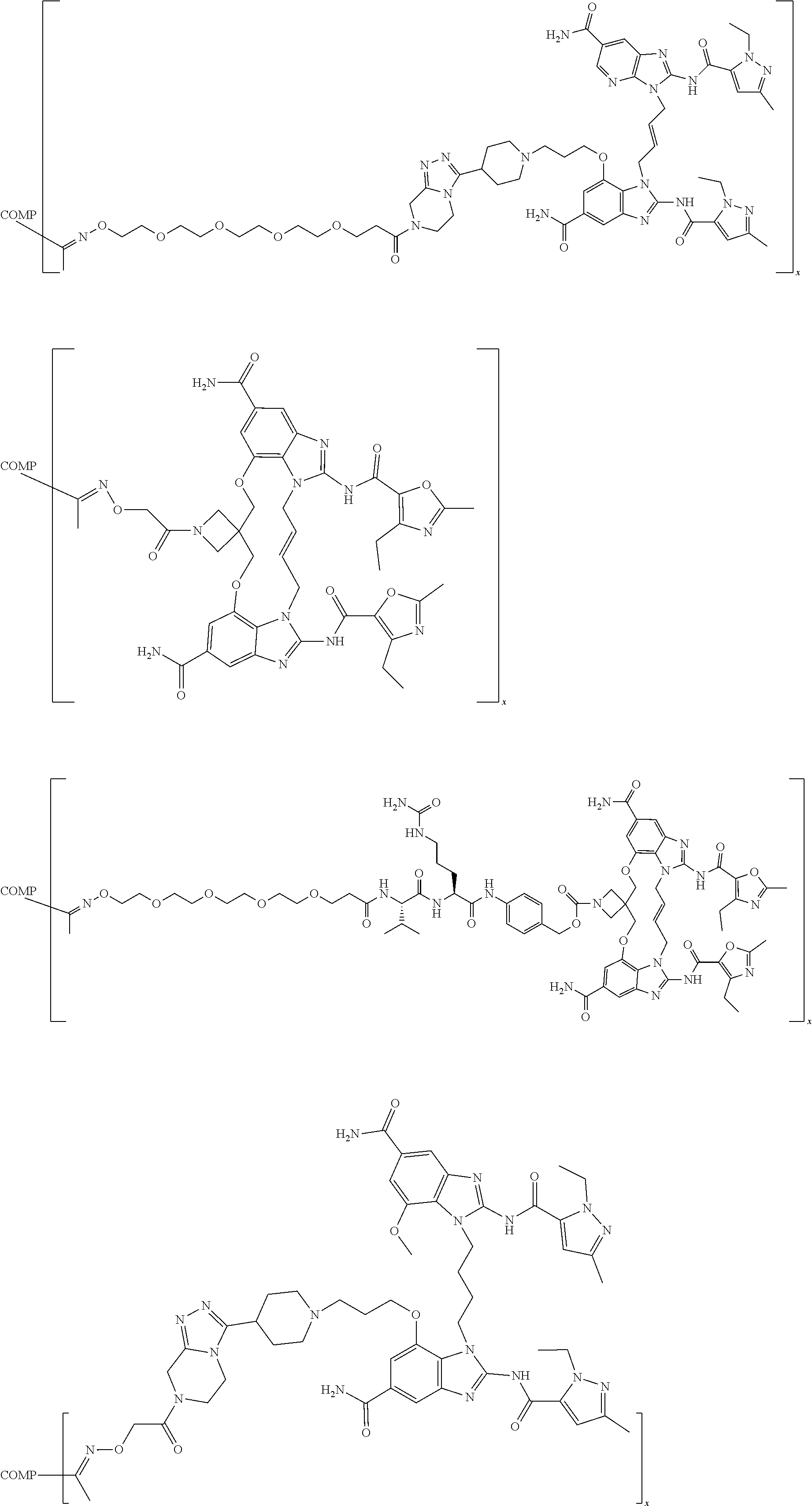 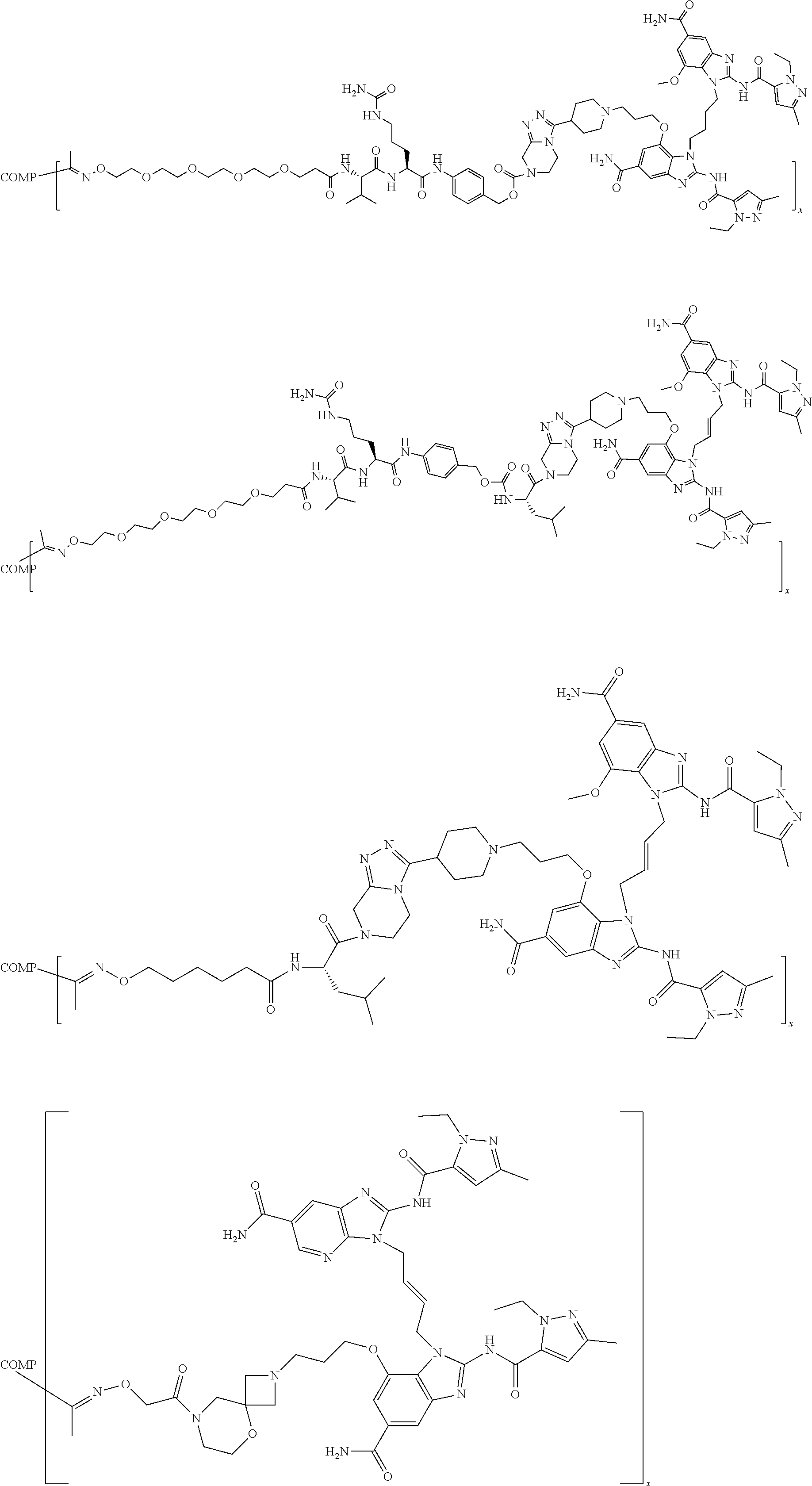 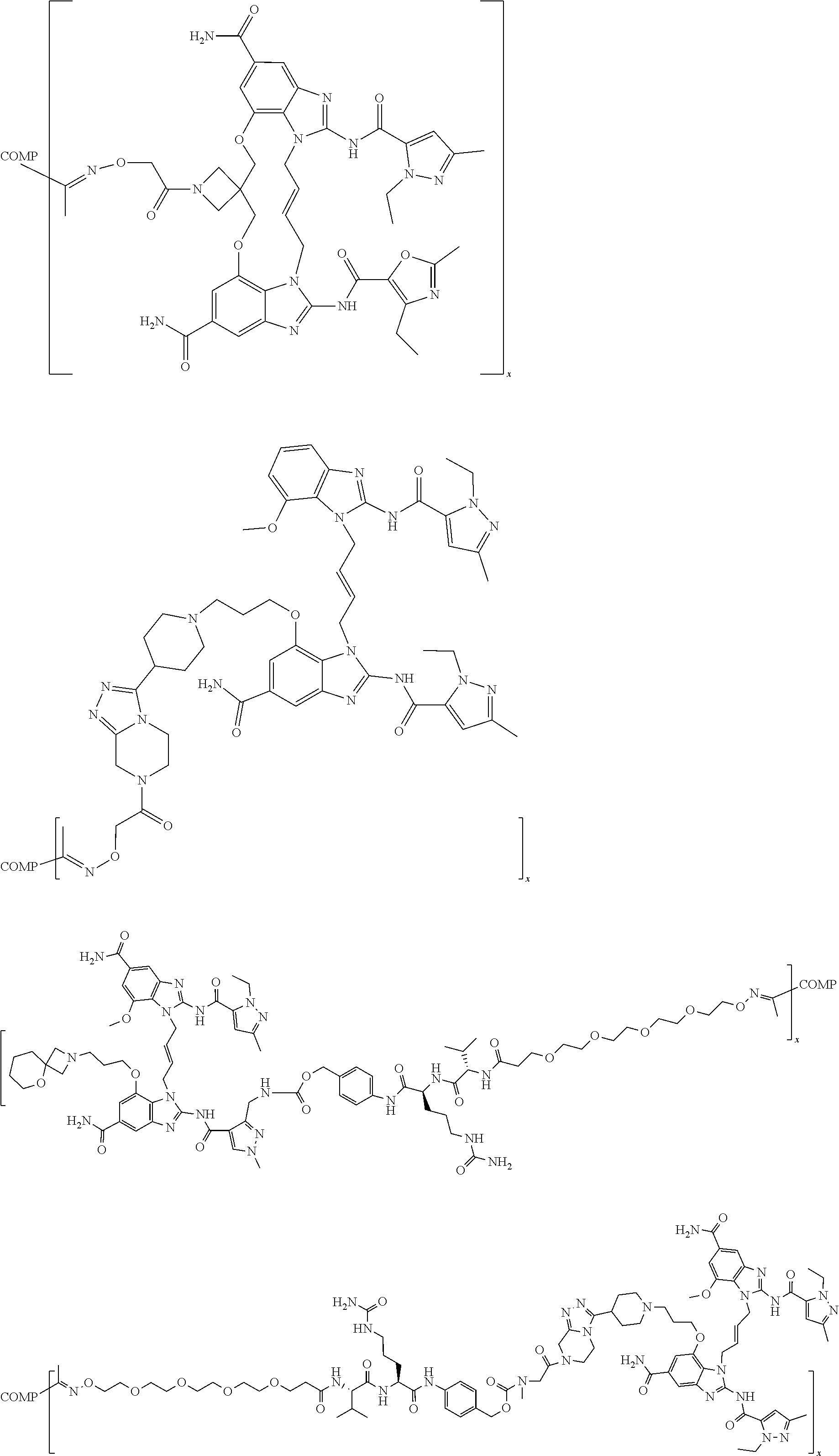 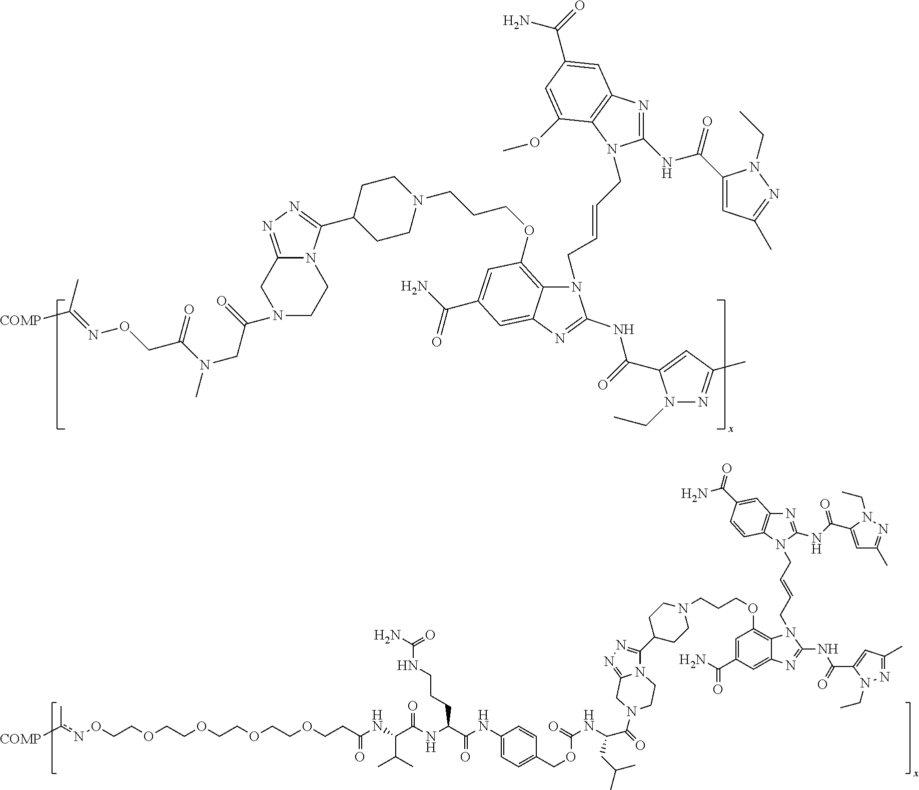
or a pharmaceutically acceptable salt or tautomer thereof, wherein x is an integer between 1 and 30, inclusive.

128. The conjugate of claim 63, wherein the conjugate is selected from the group consisting of: 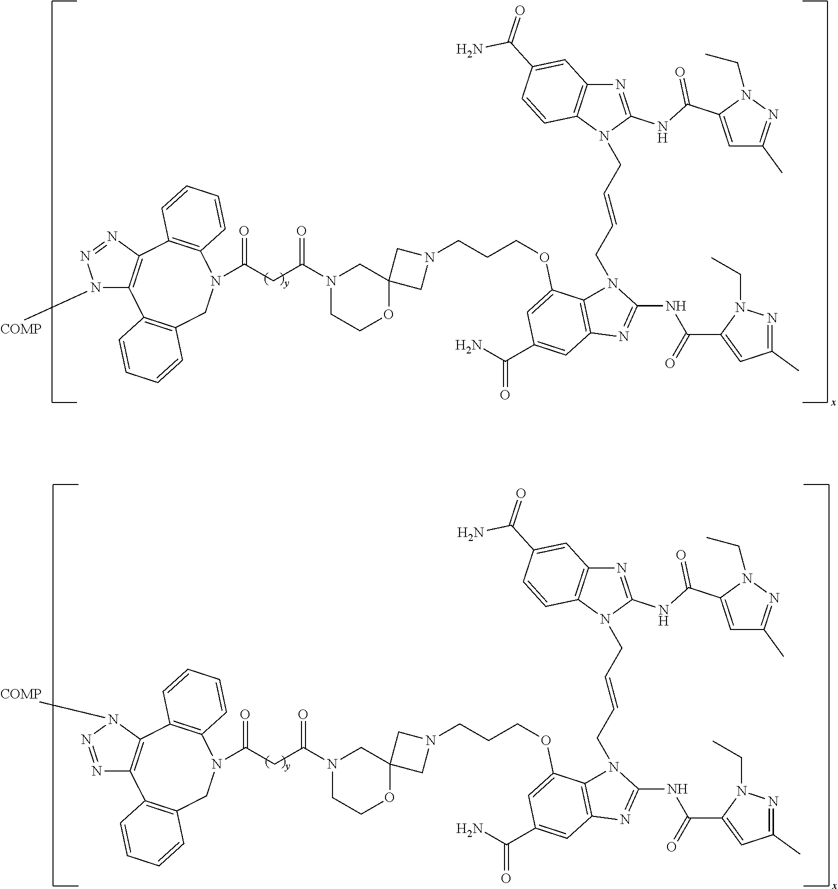 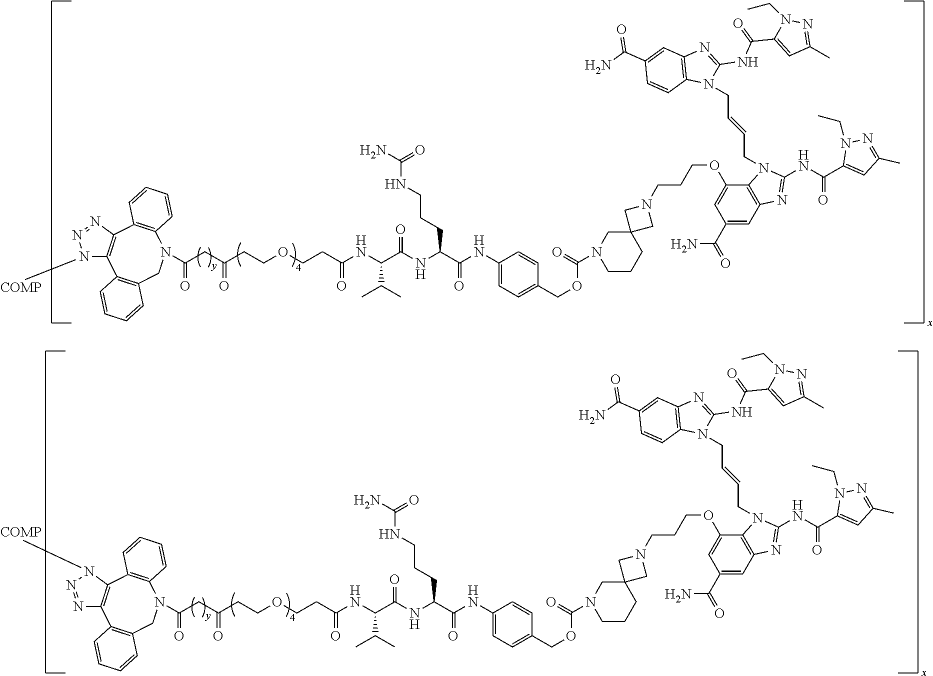 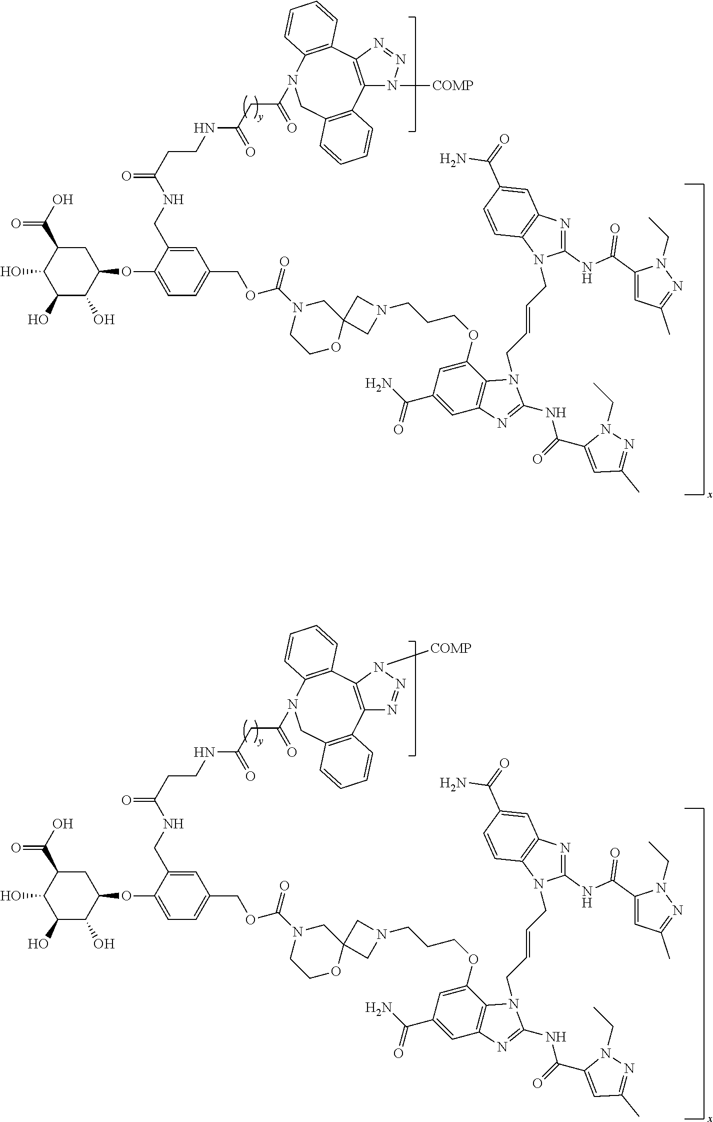 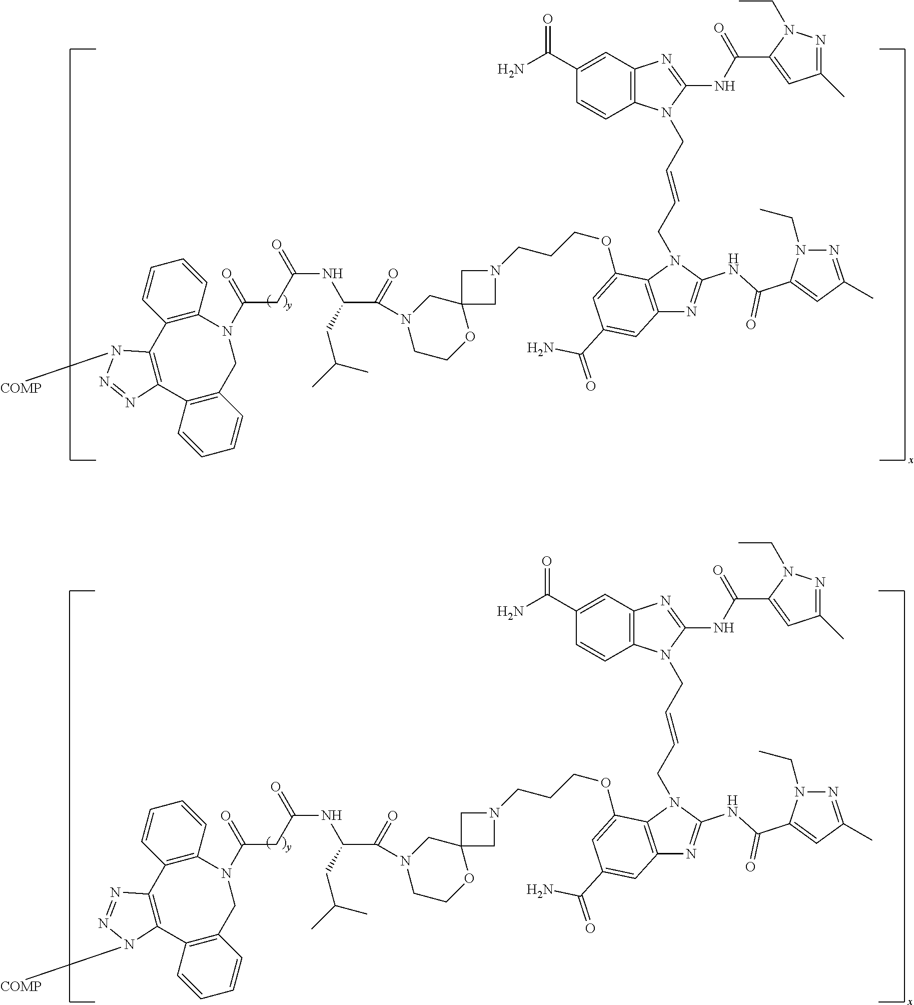 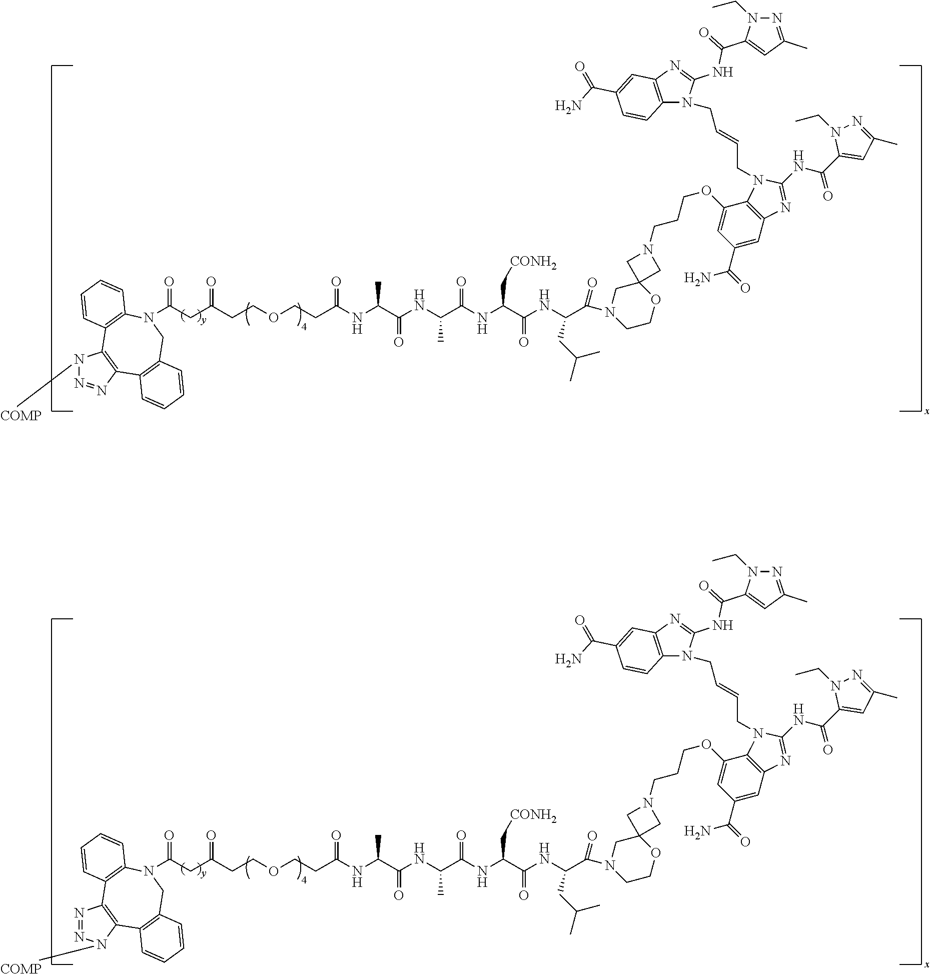 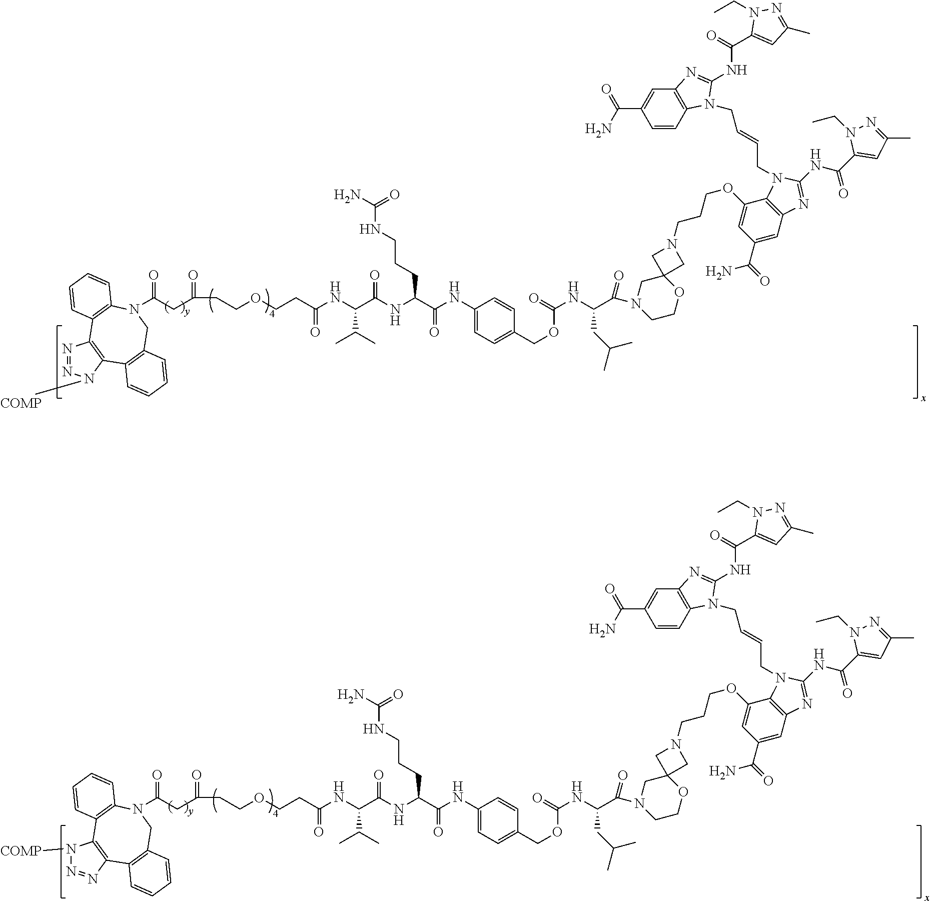 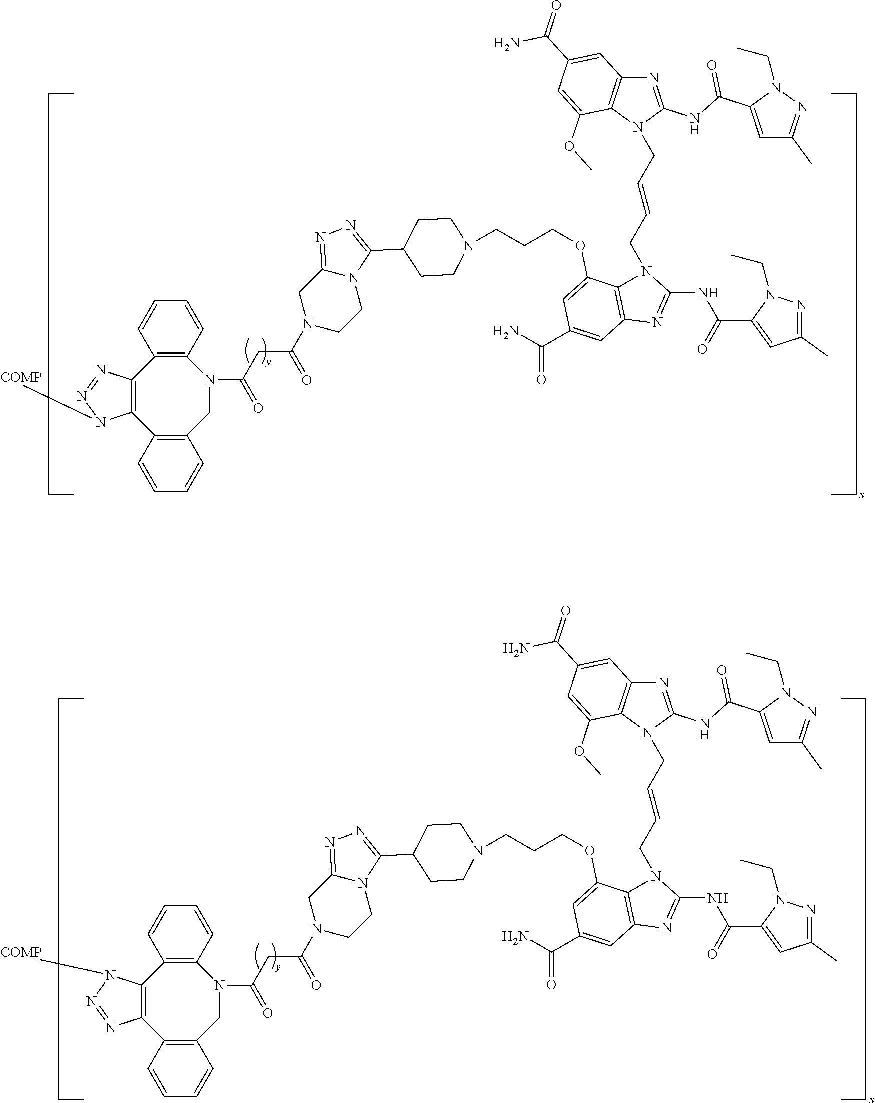 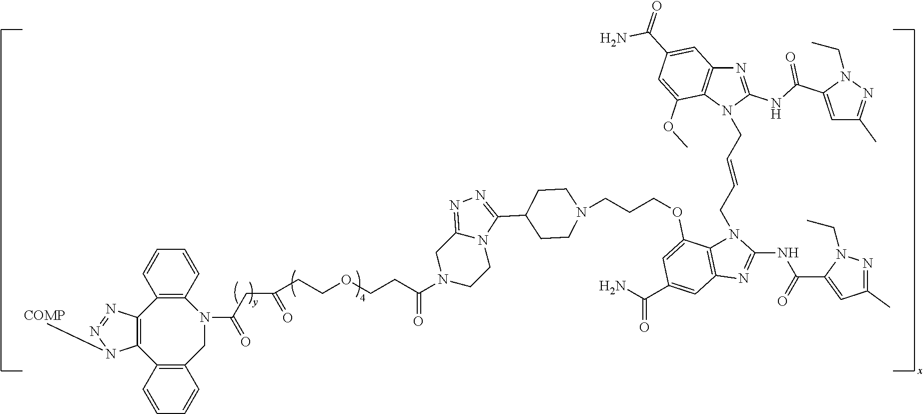 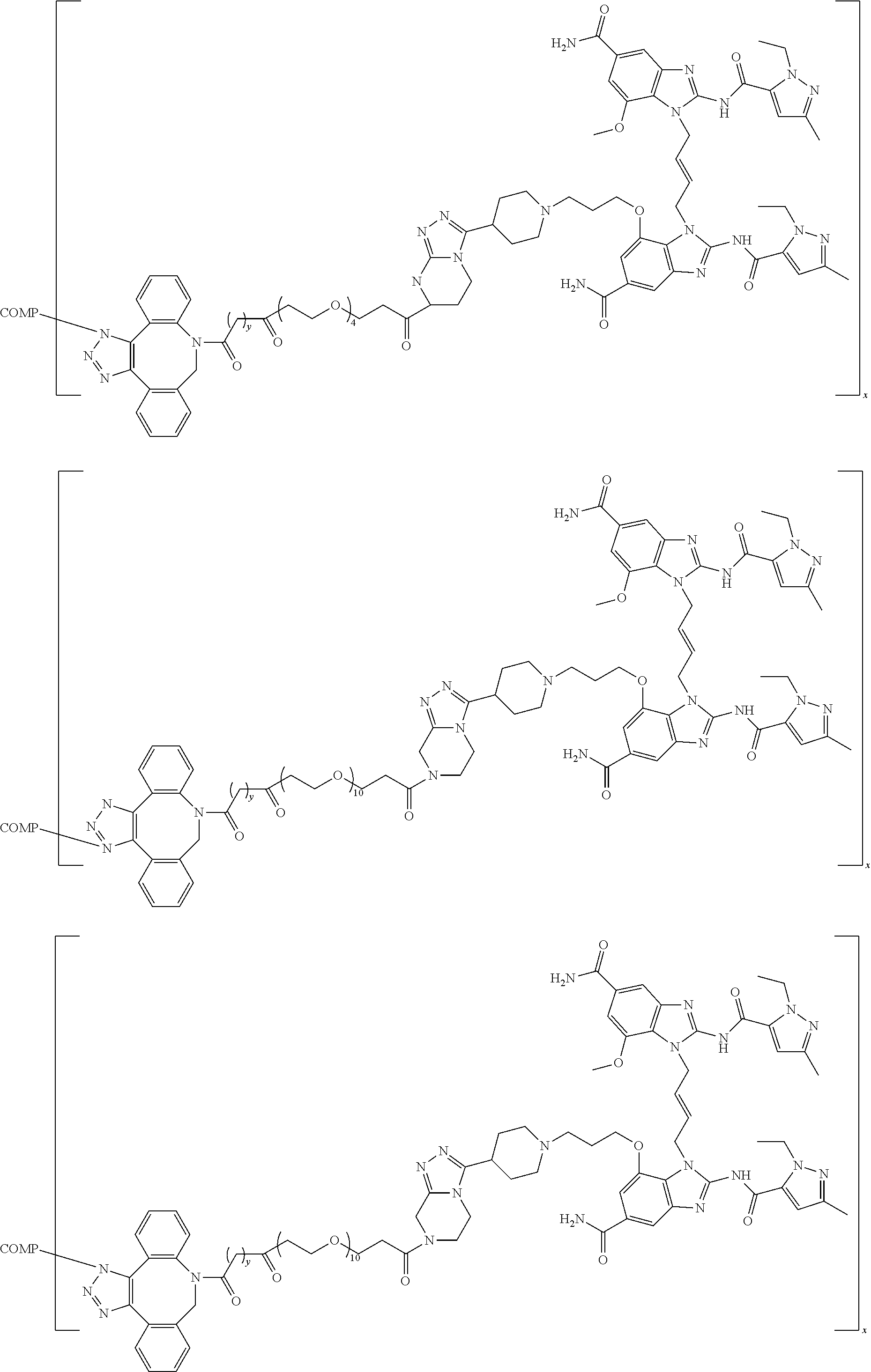 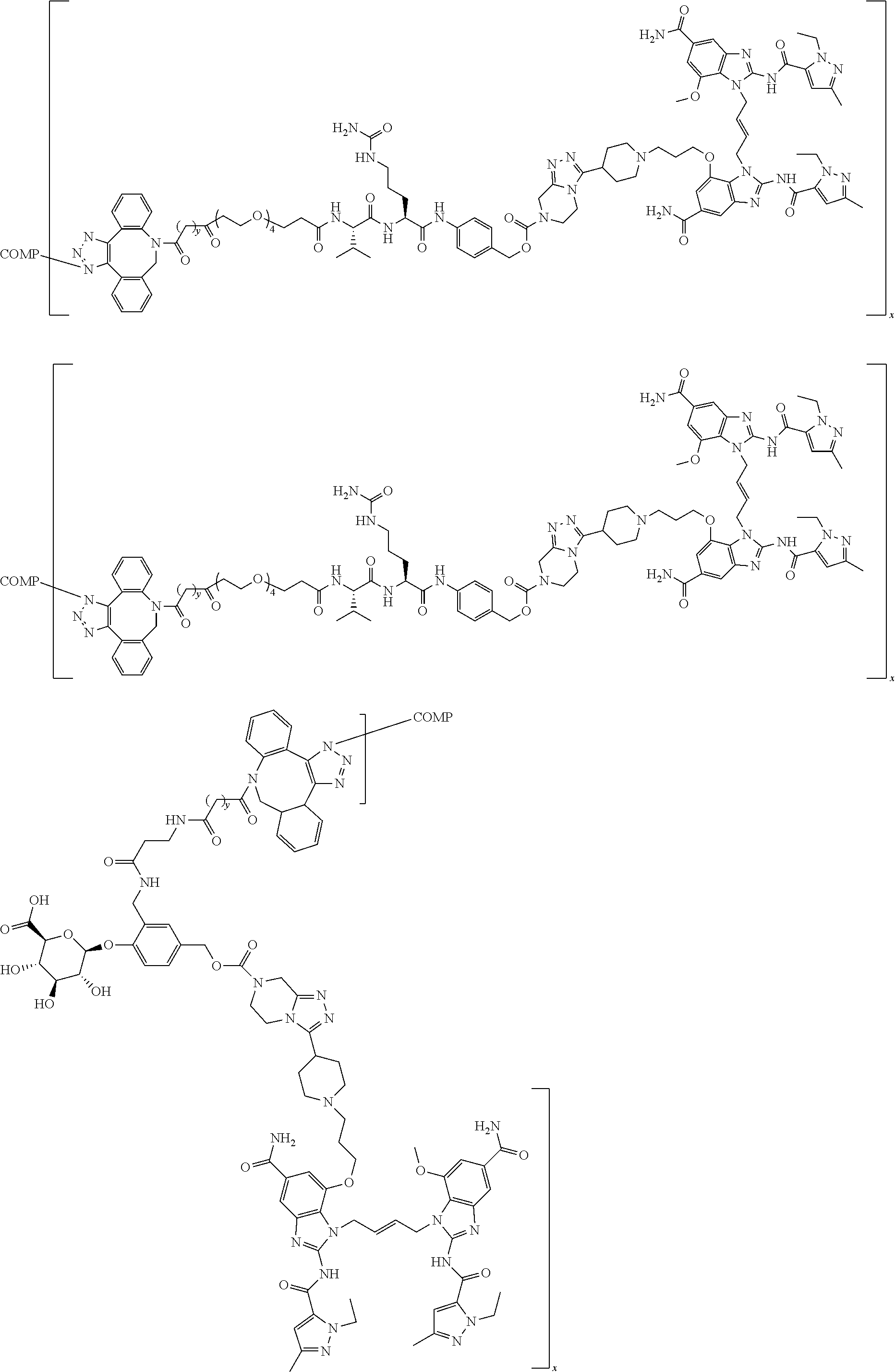 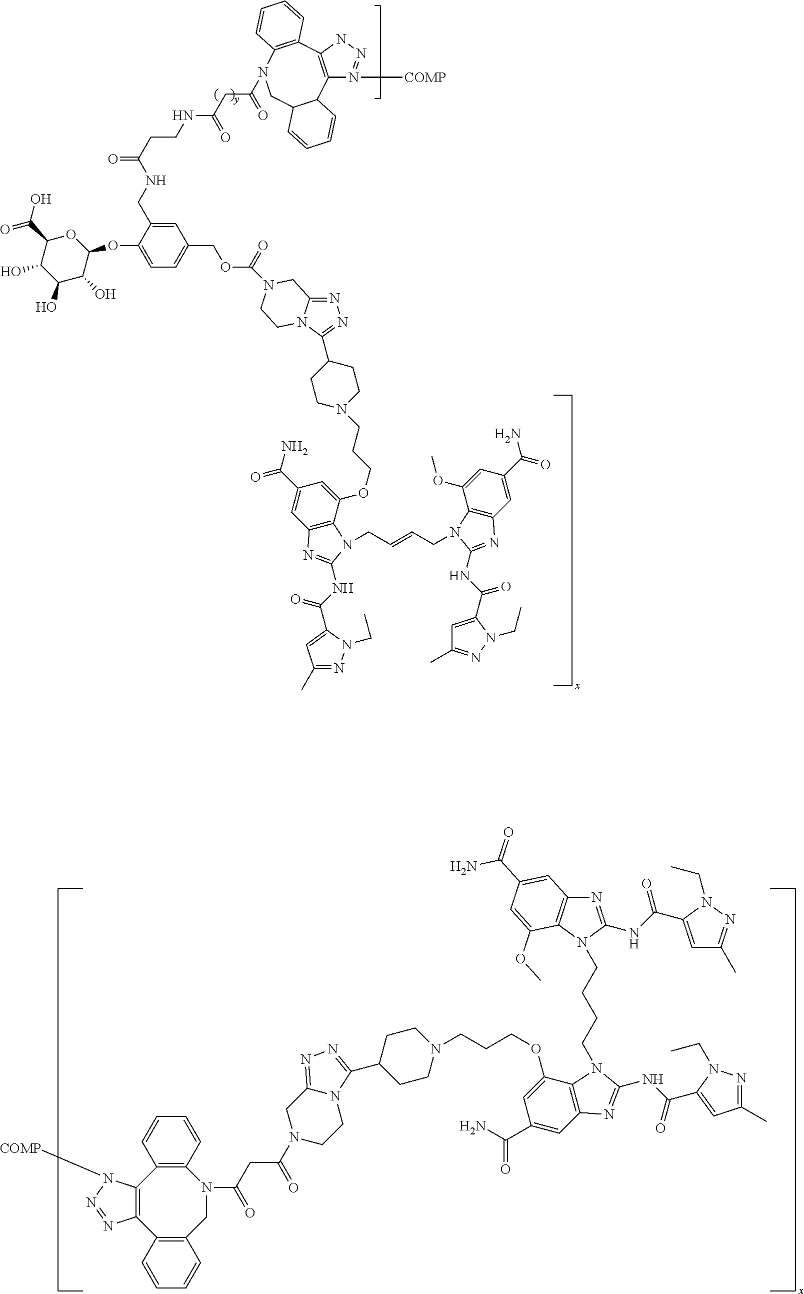 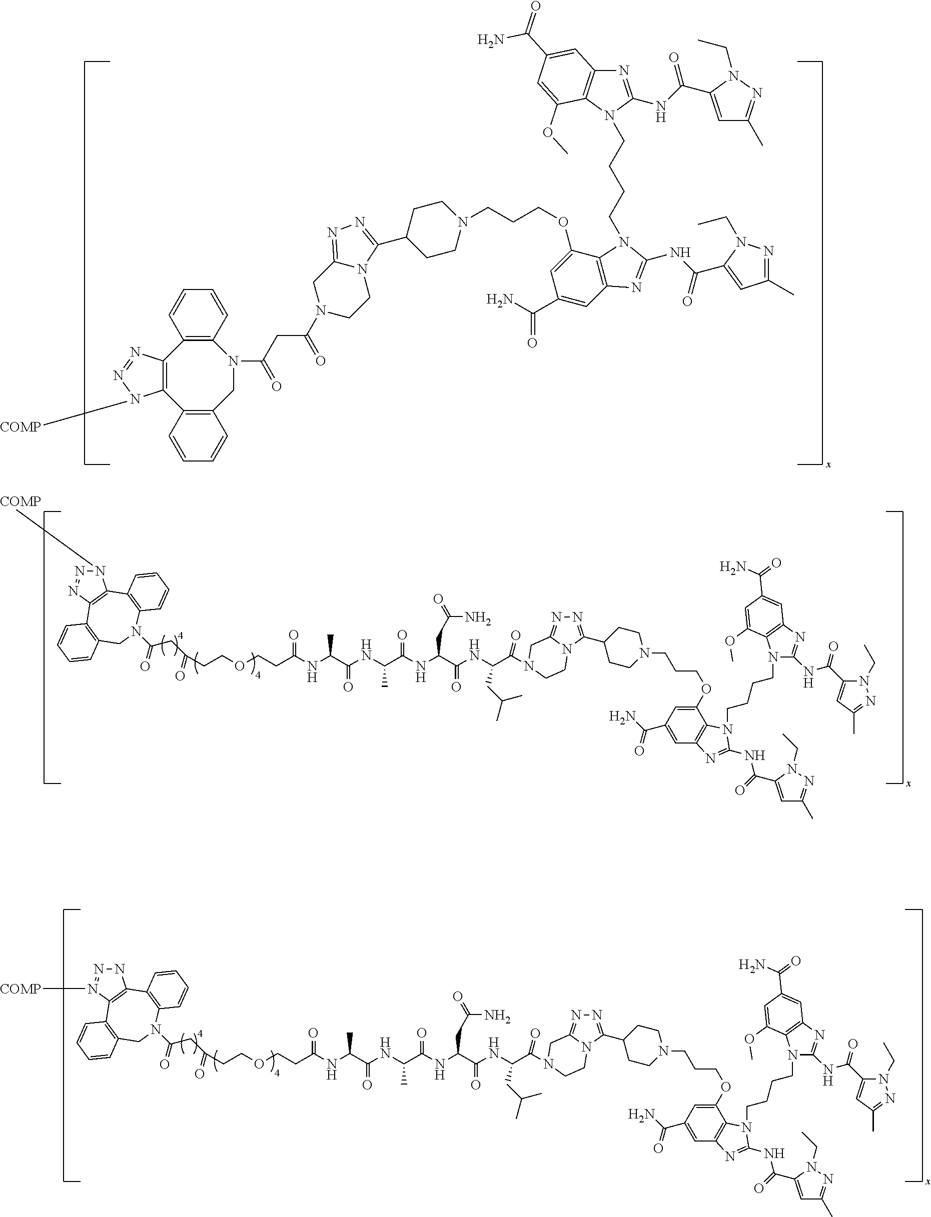 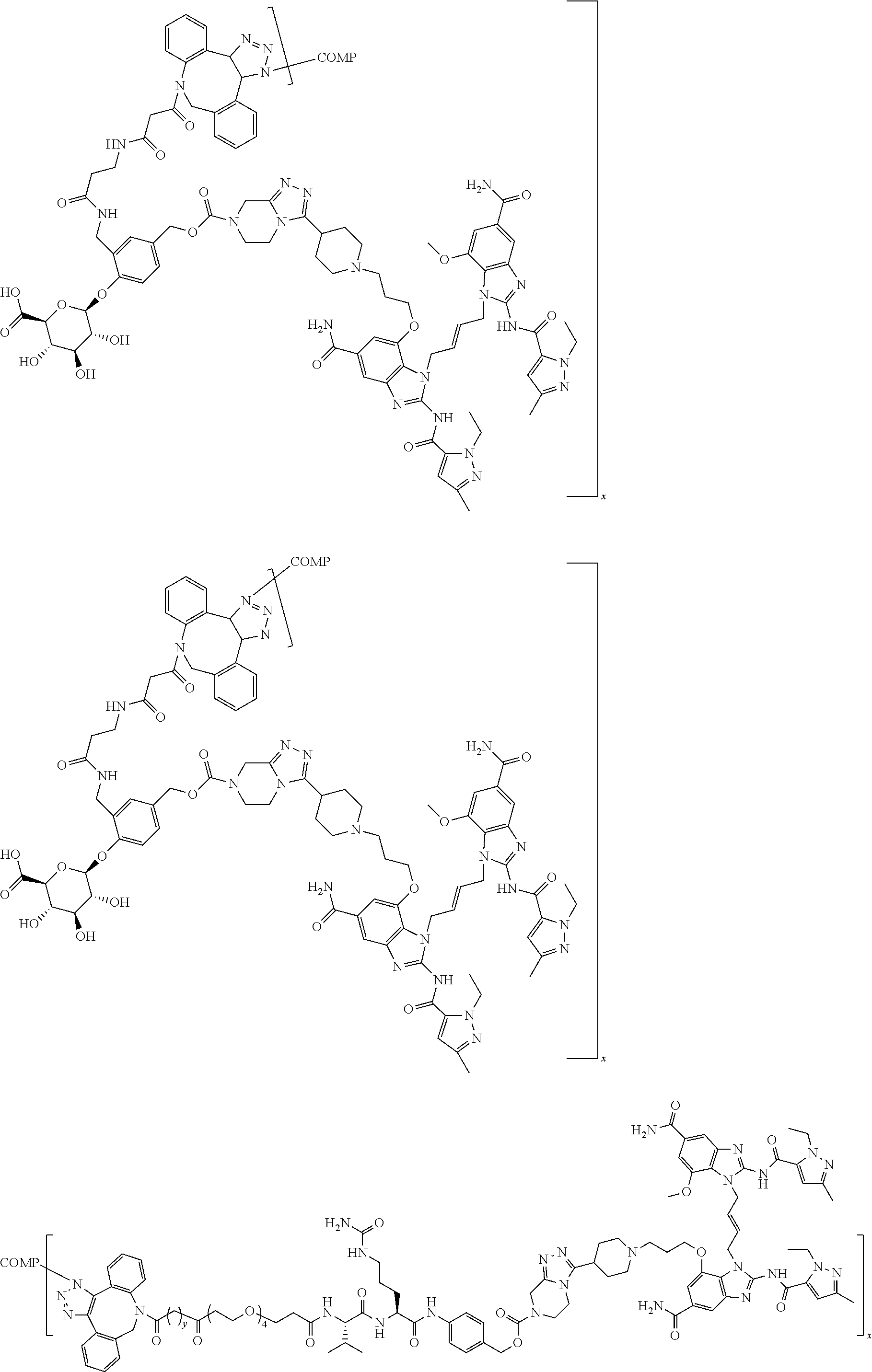 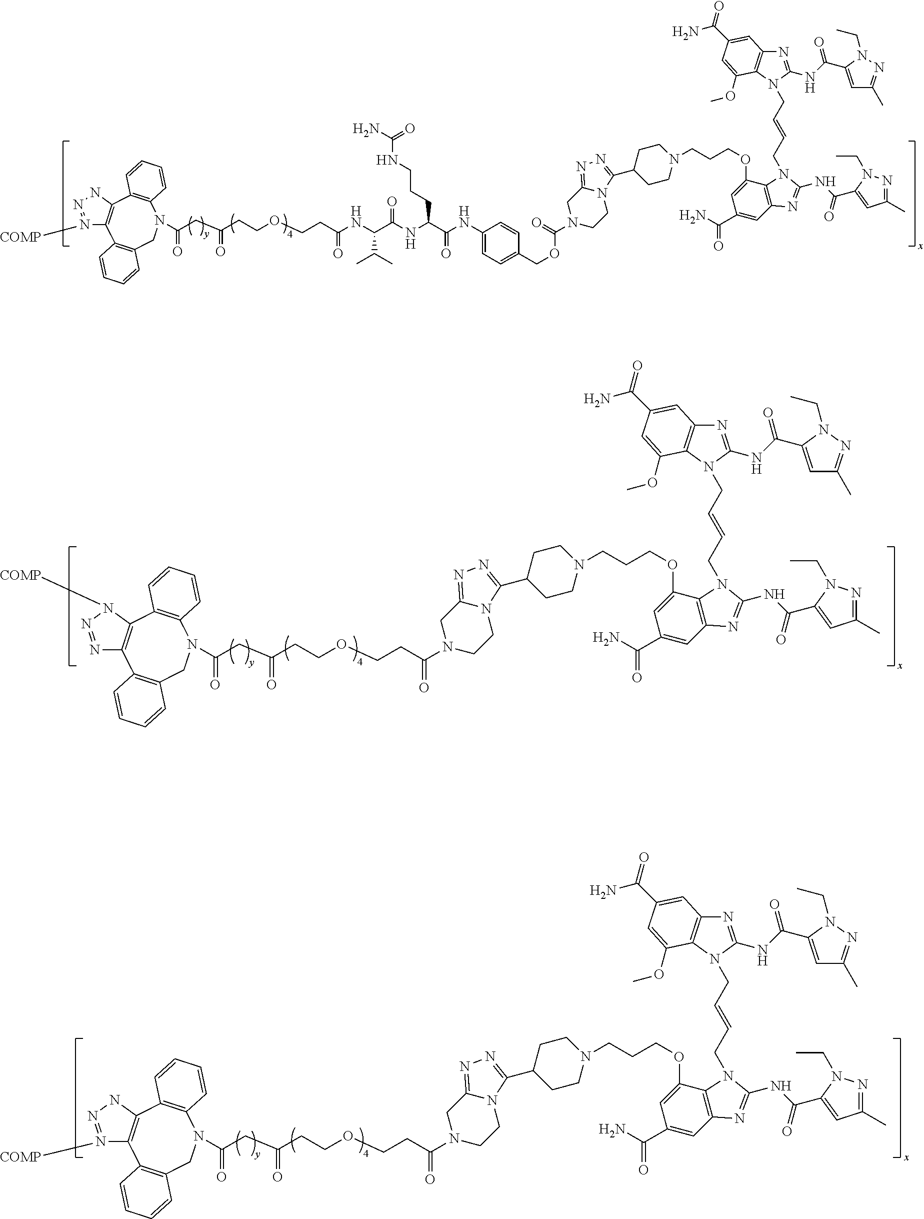 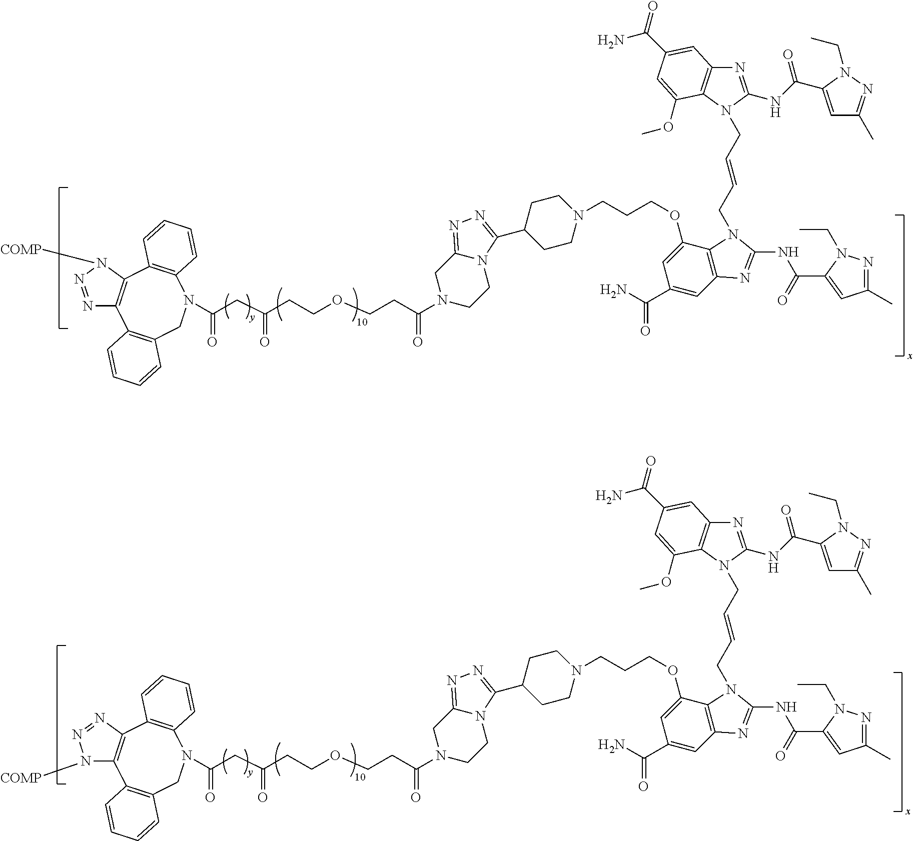 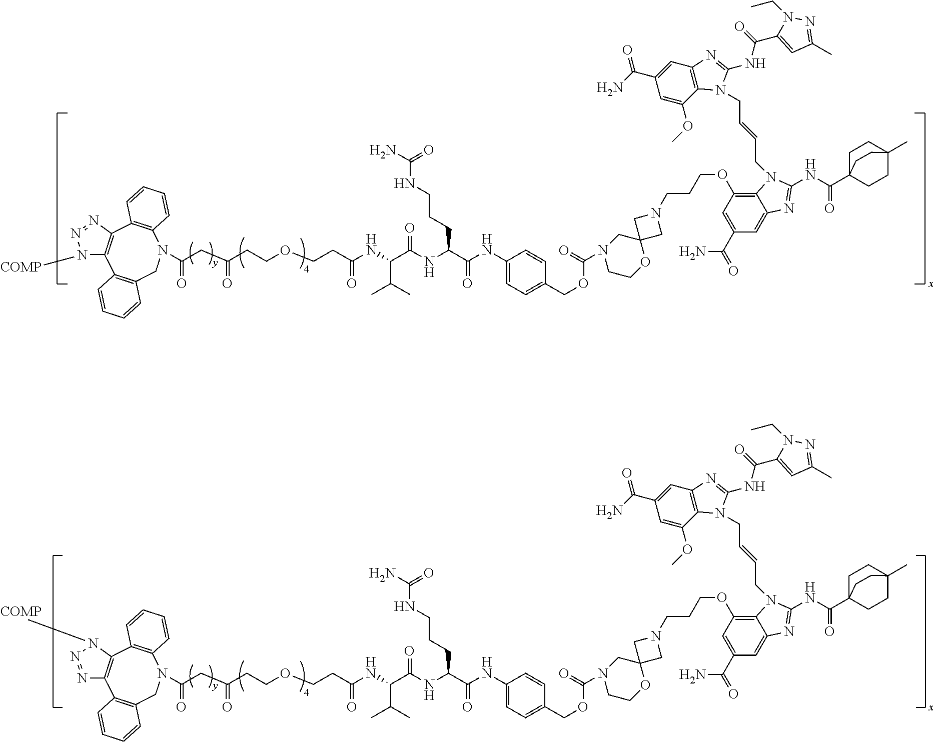 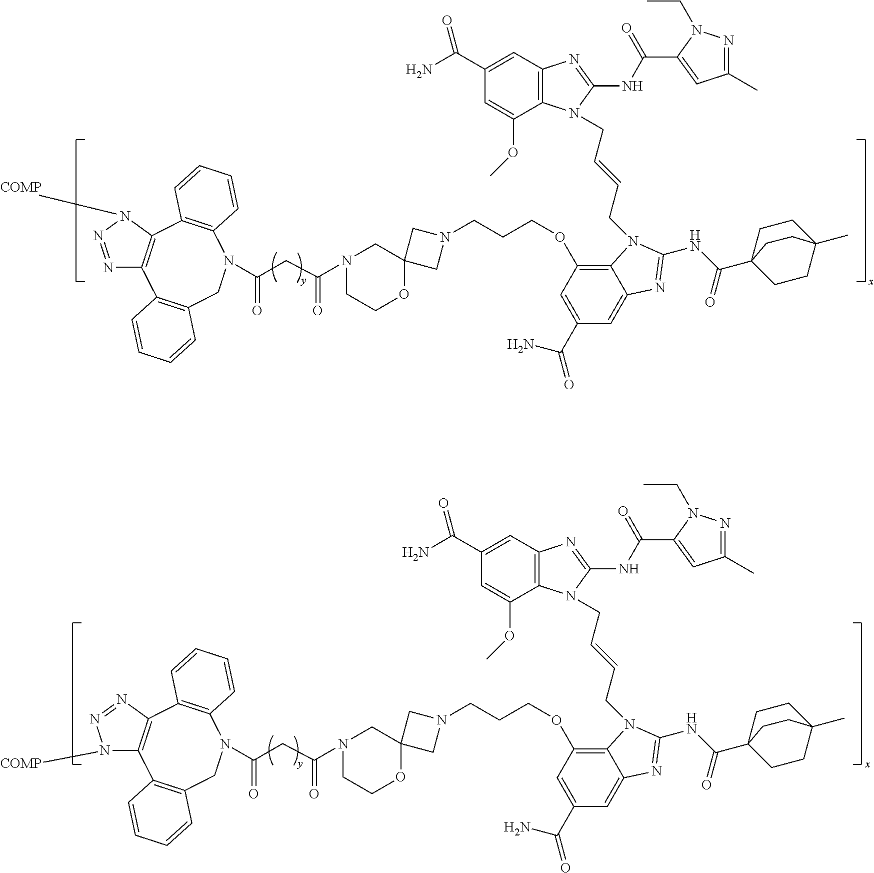 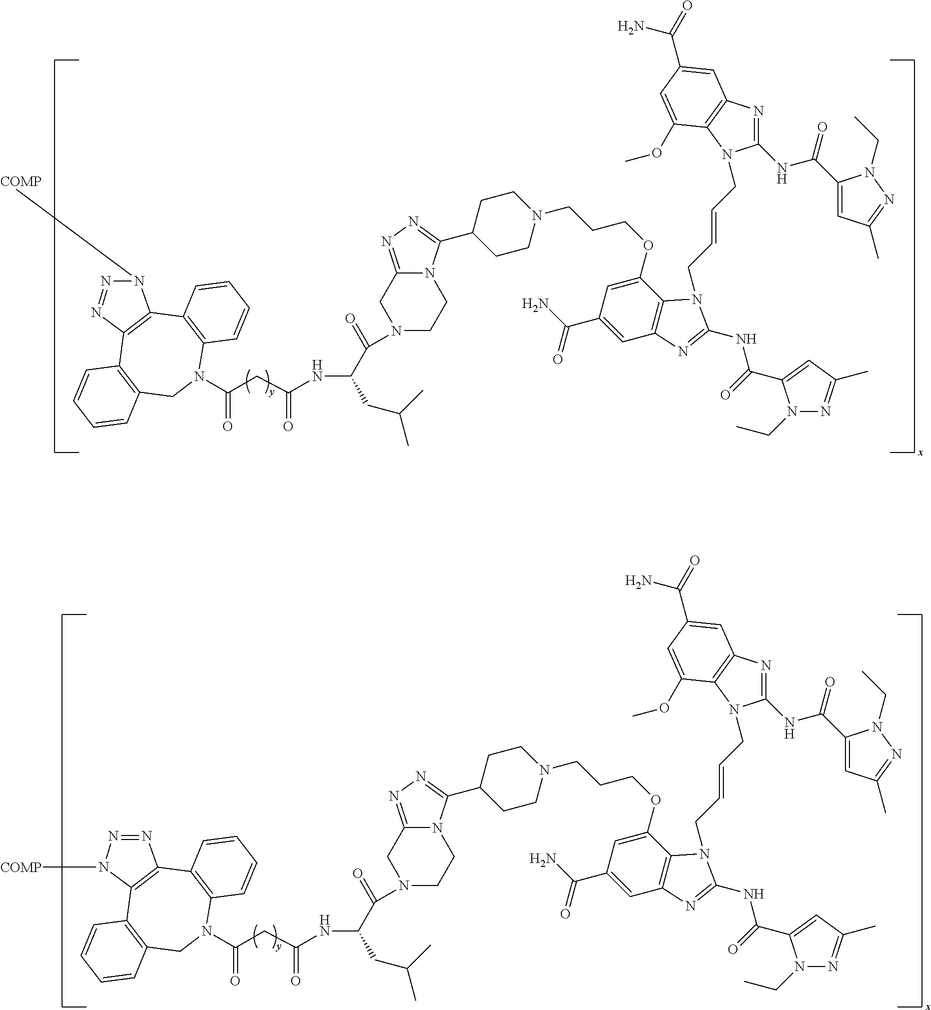 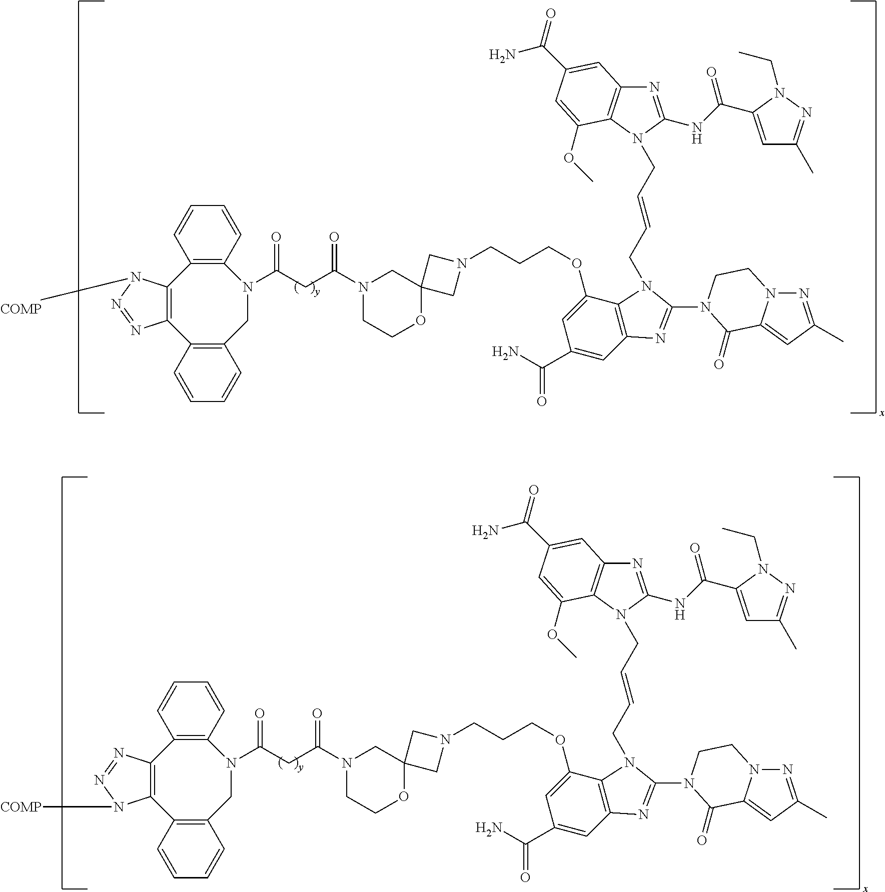 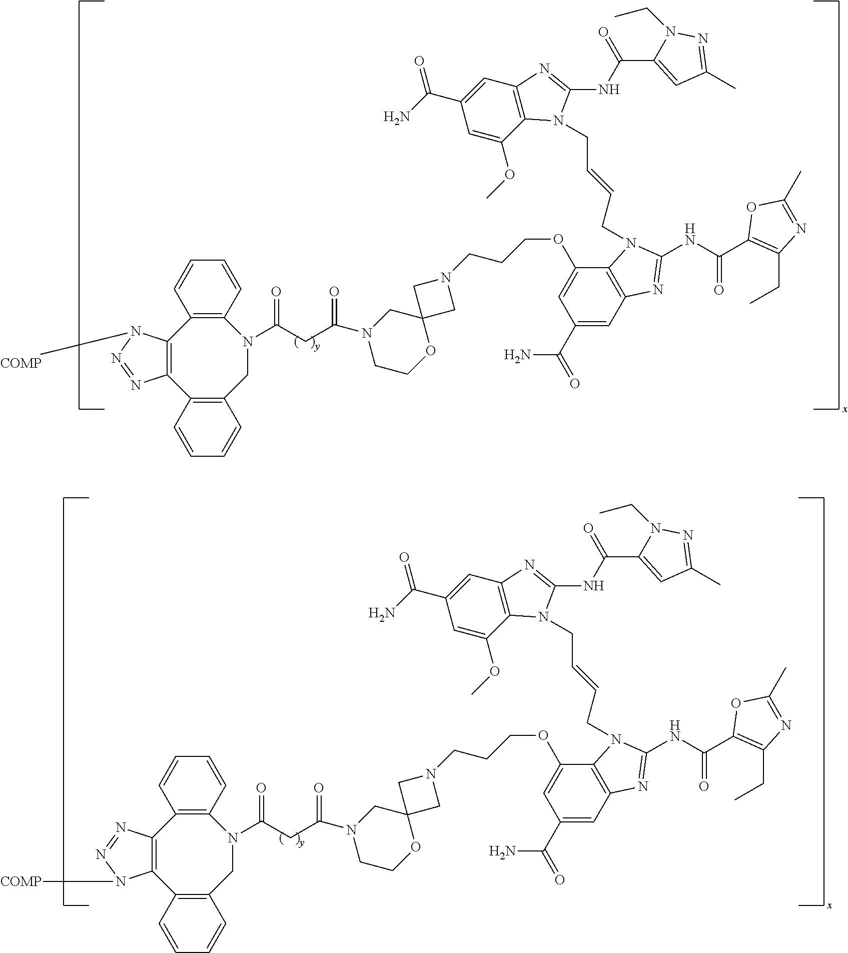 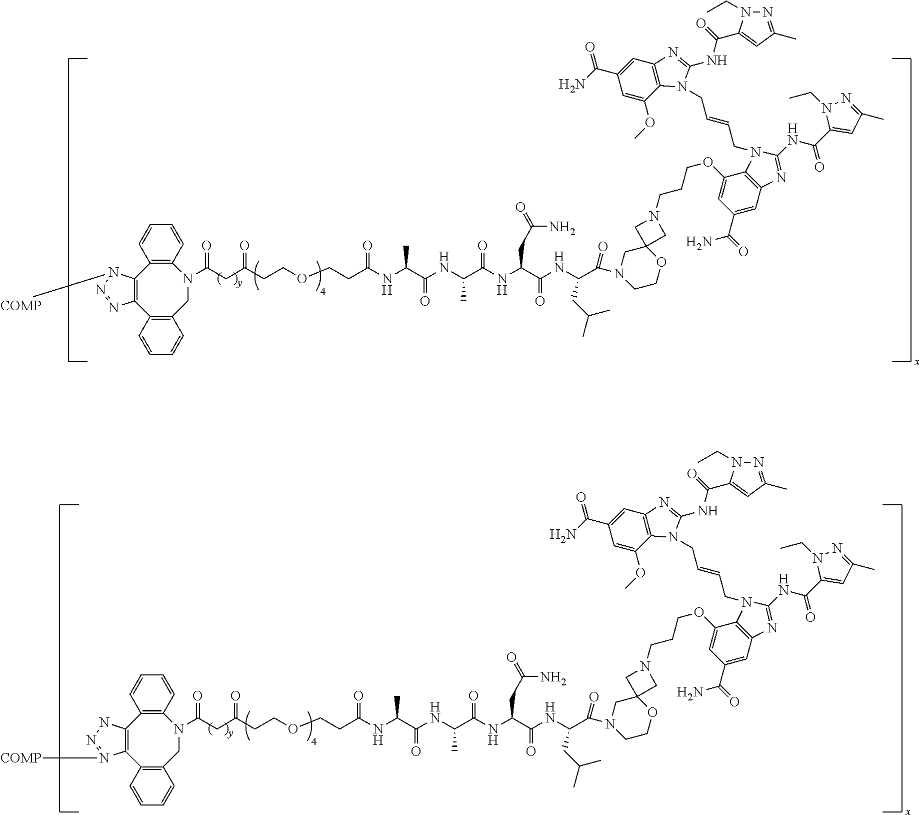 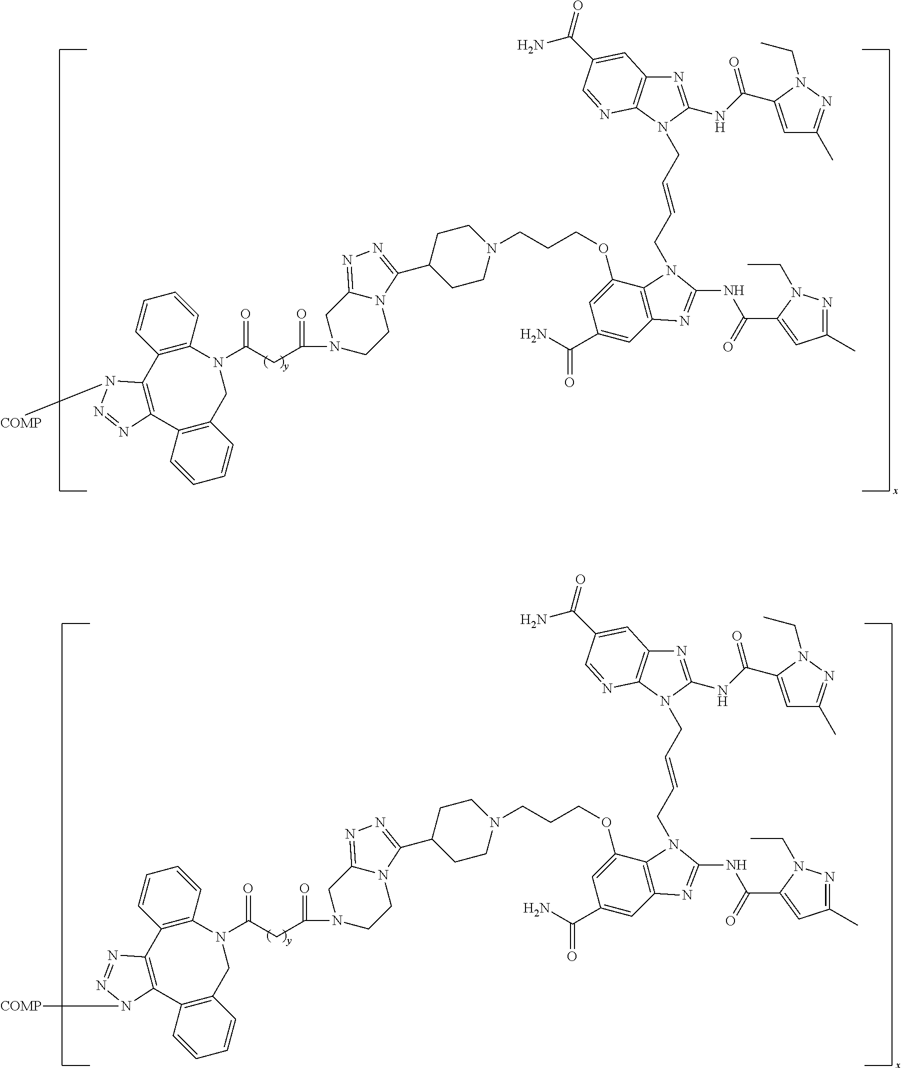 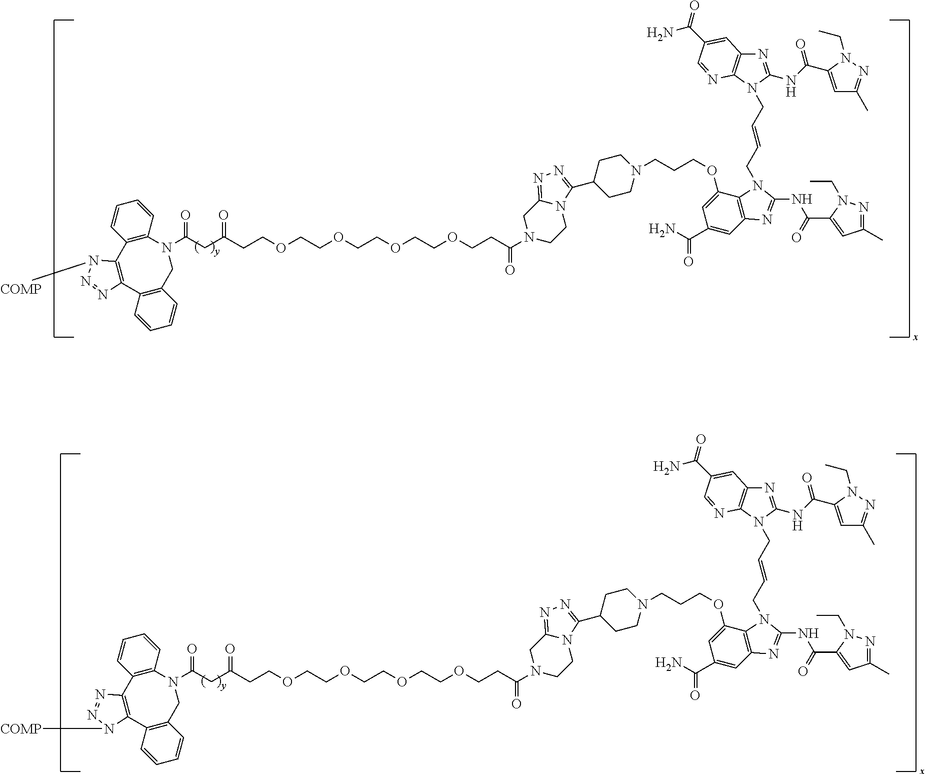 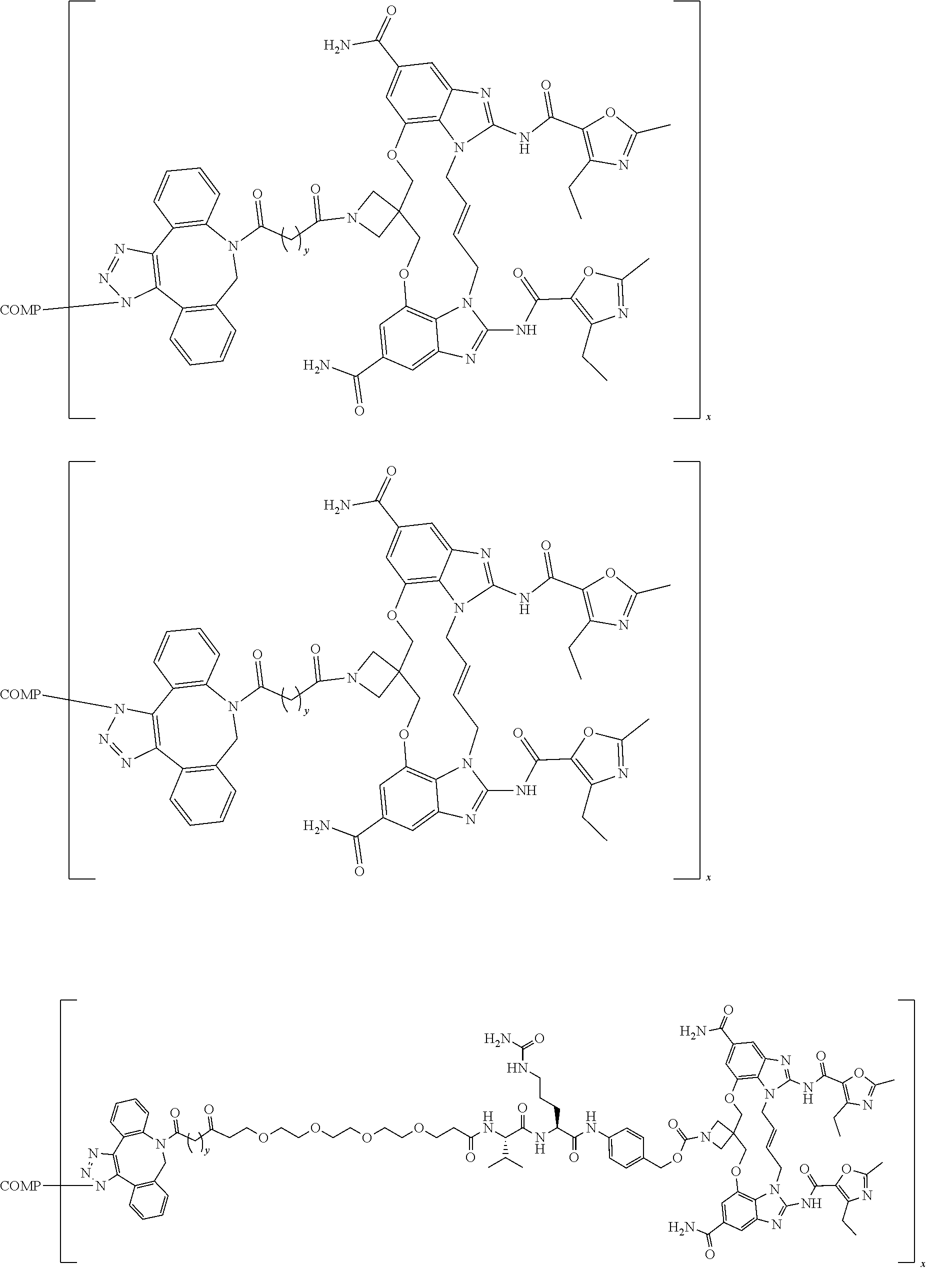 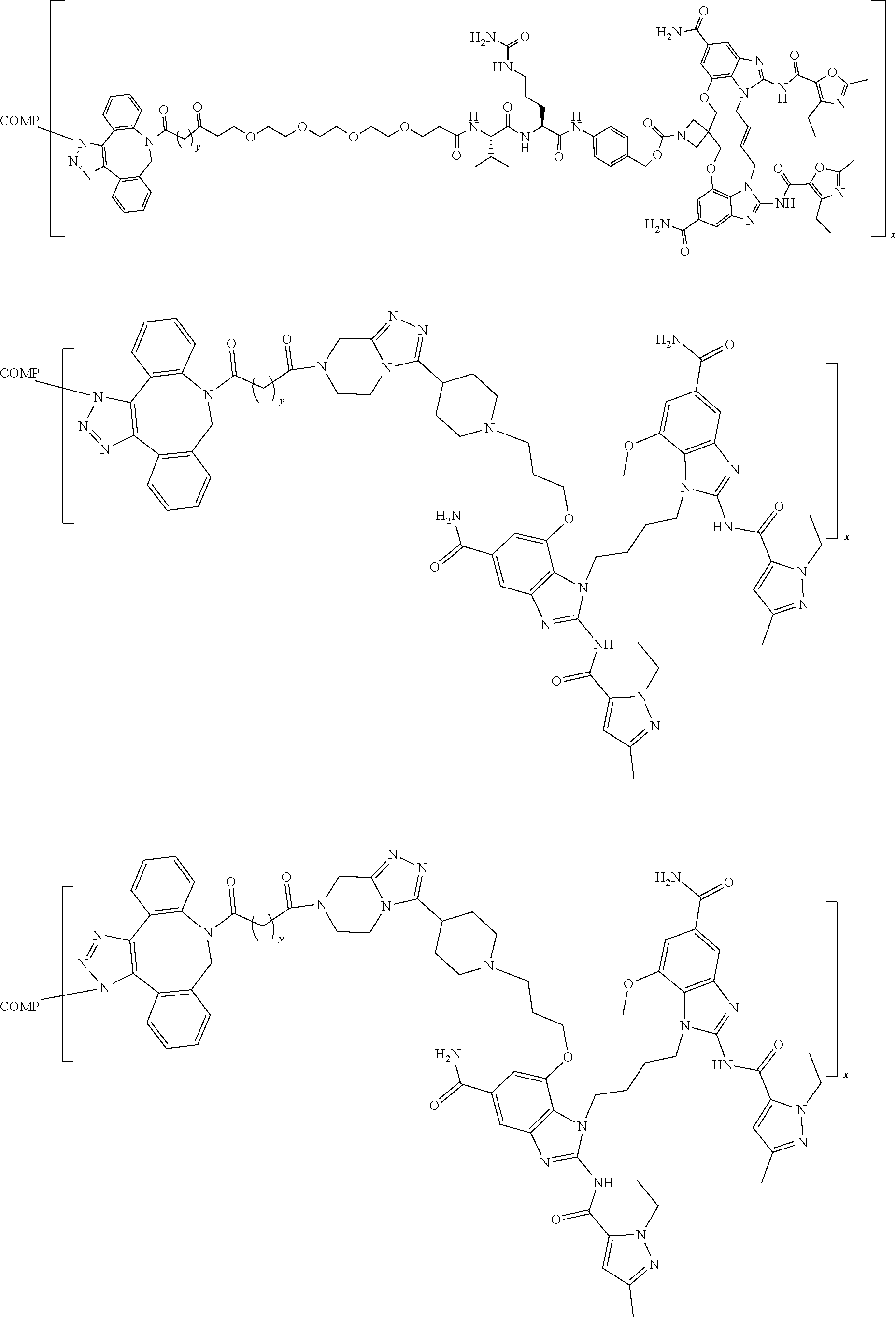 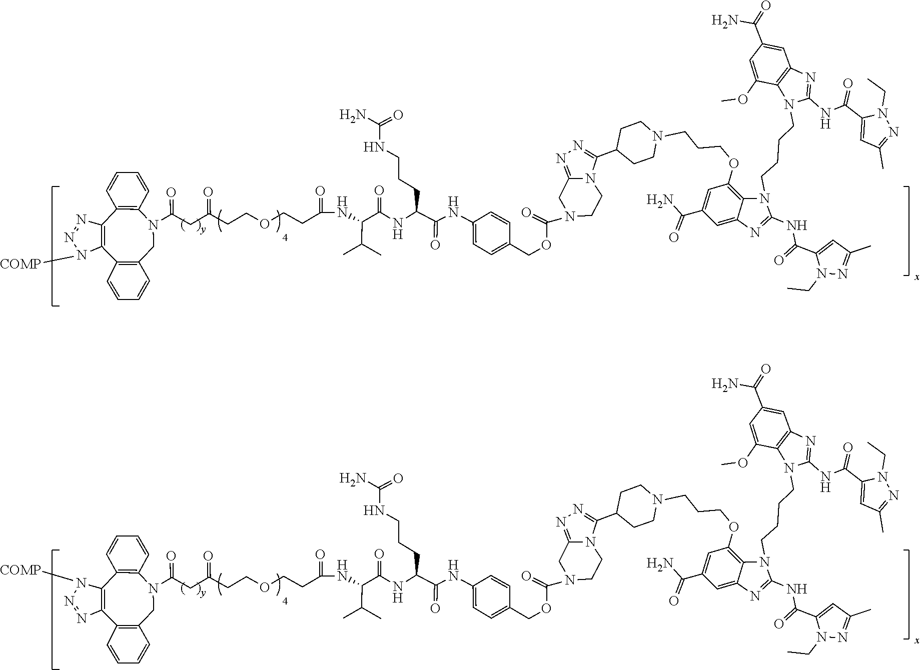 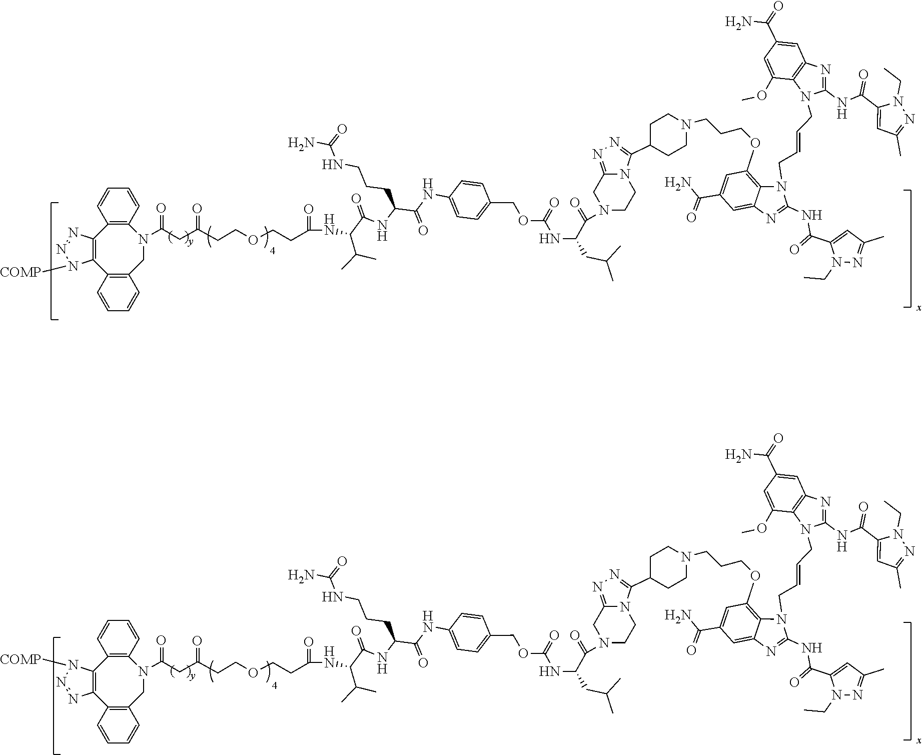 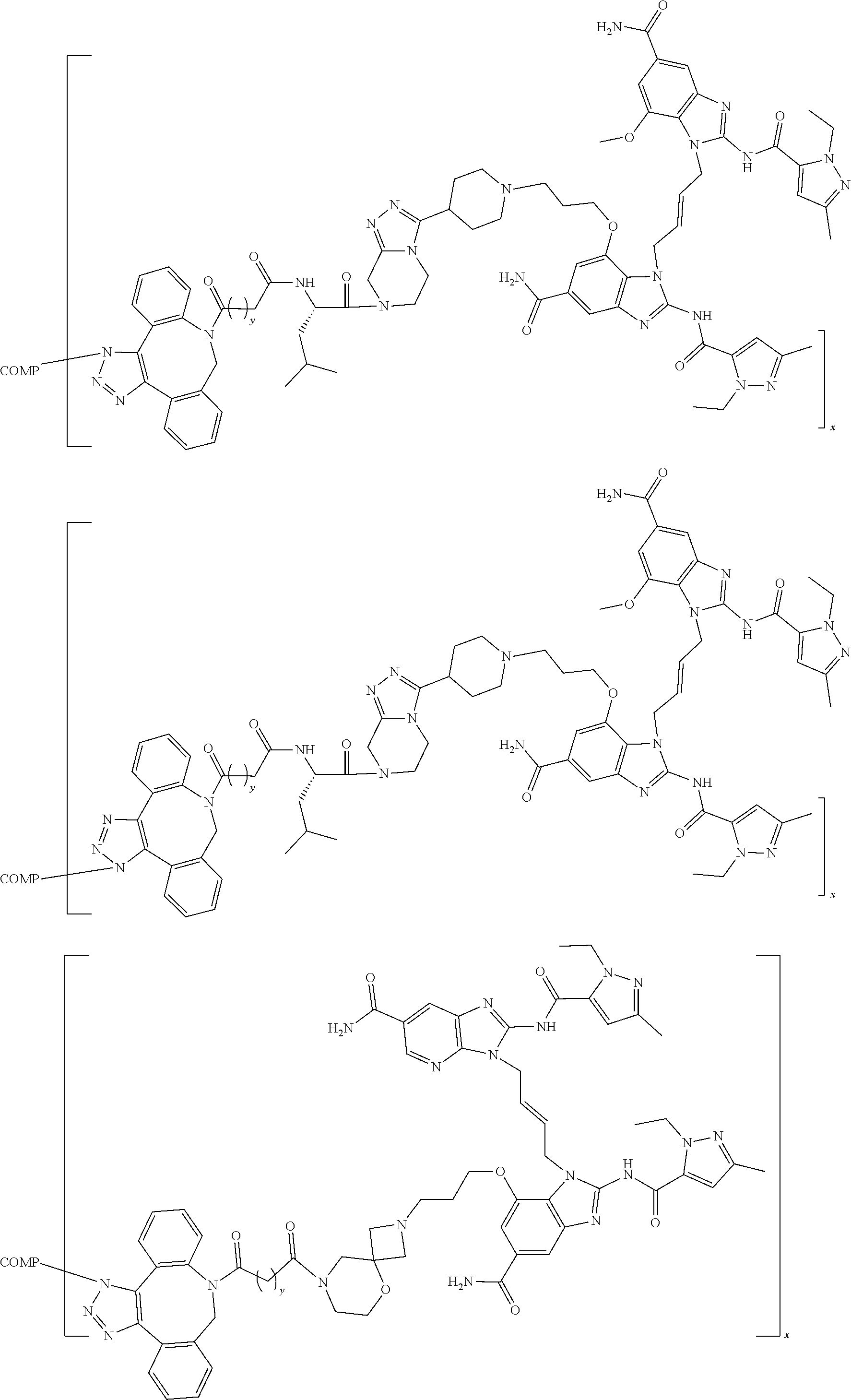 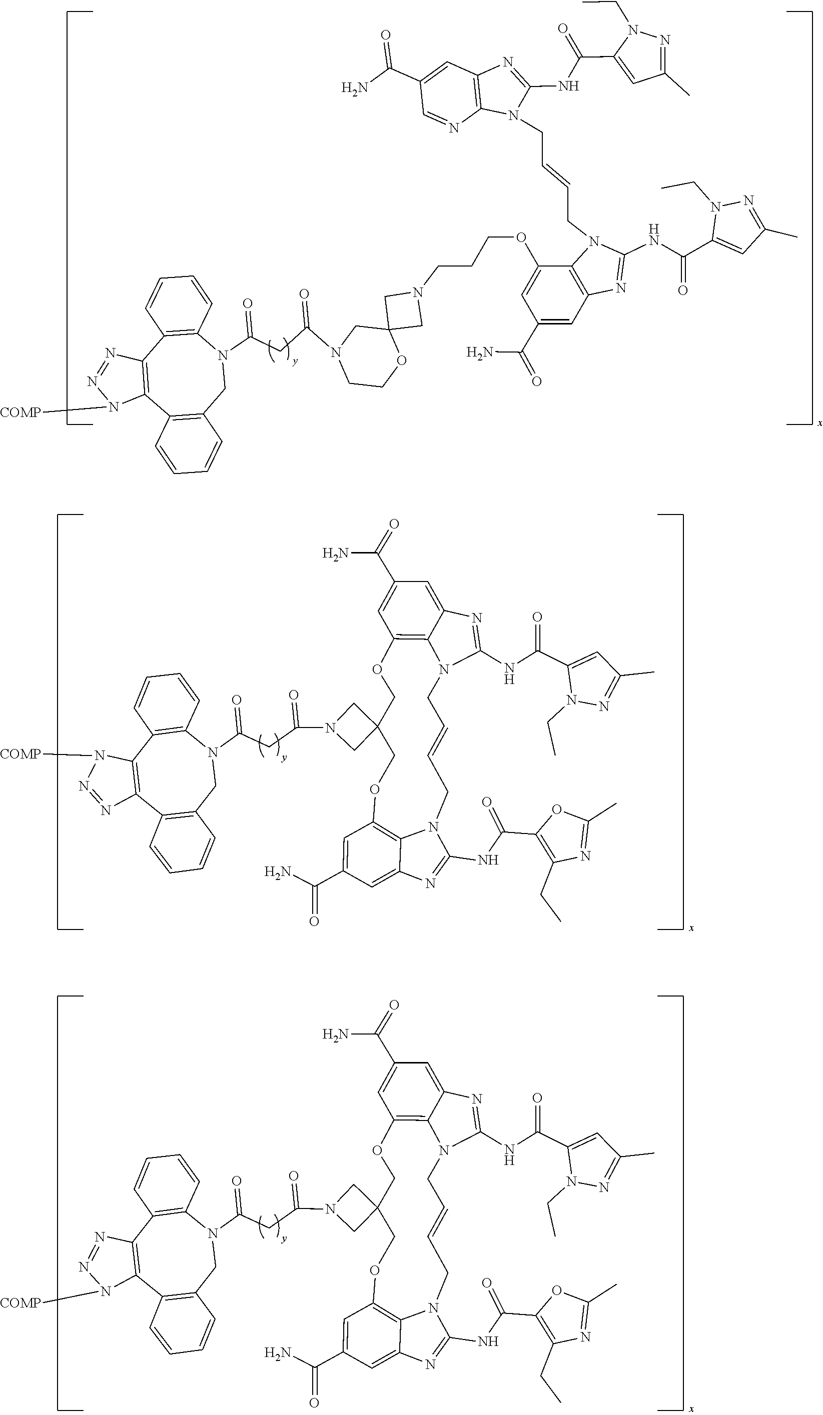 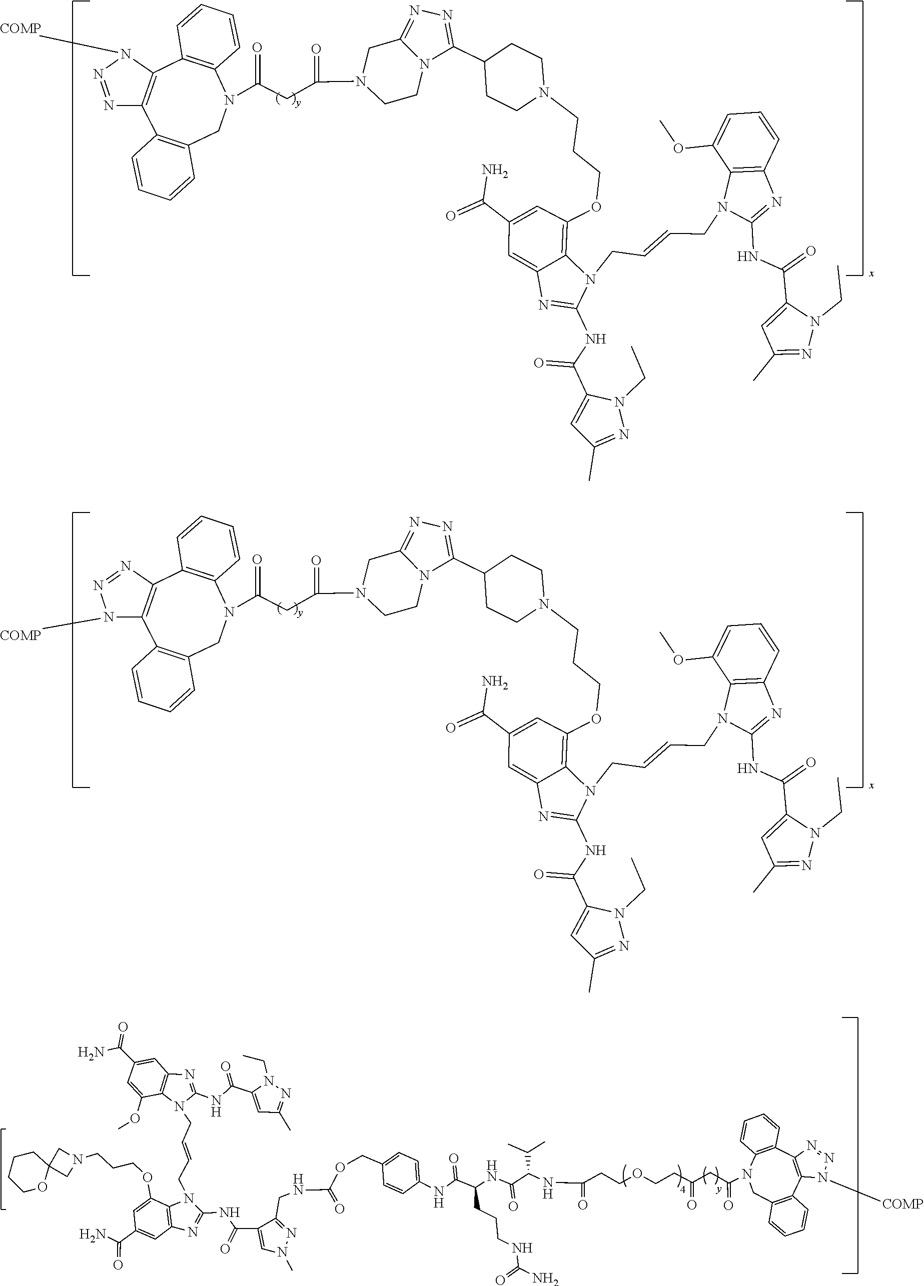 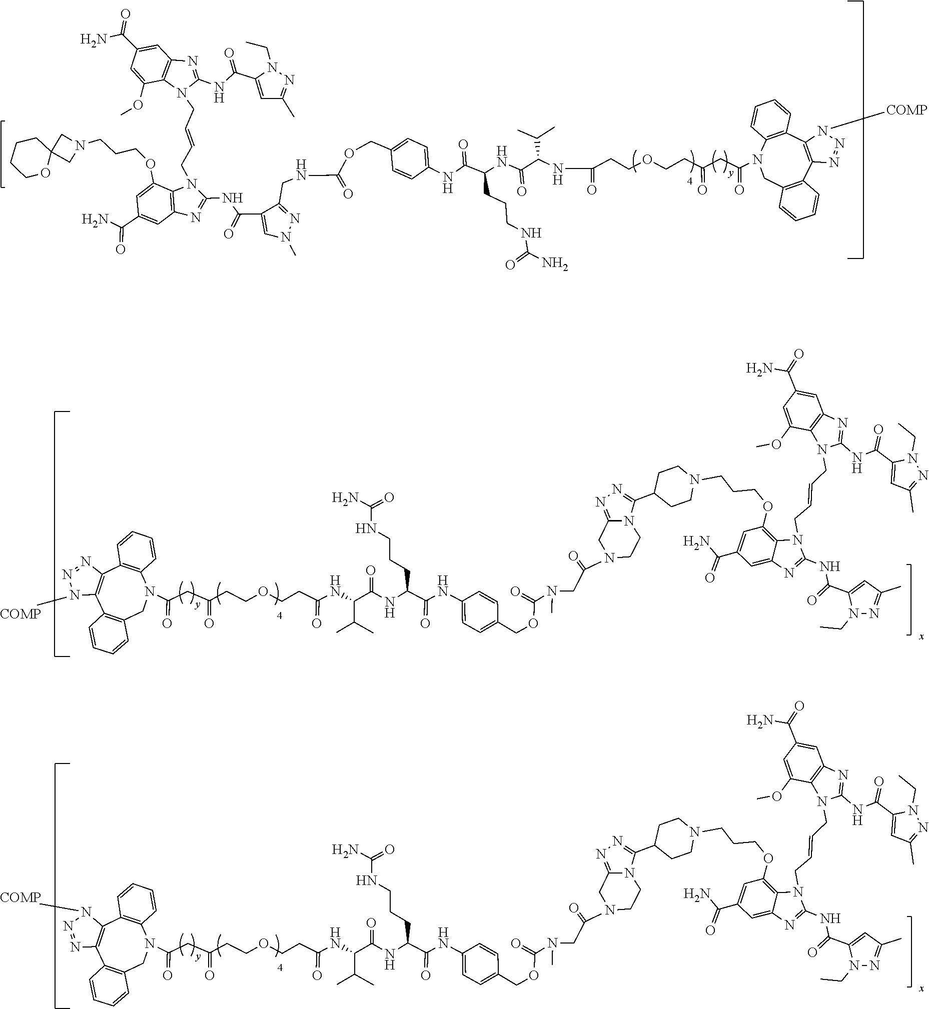 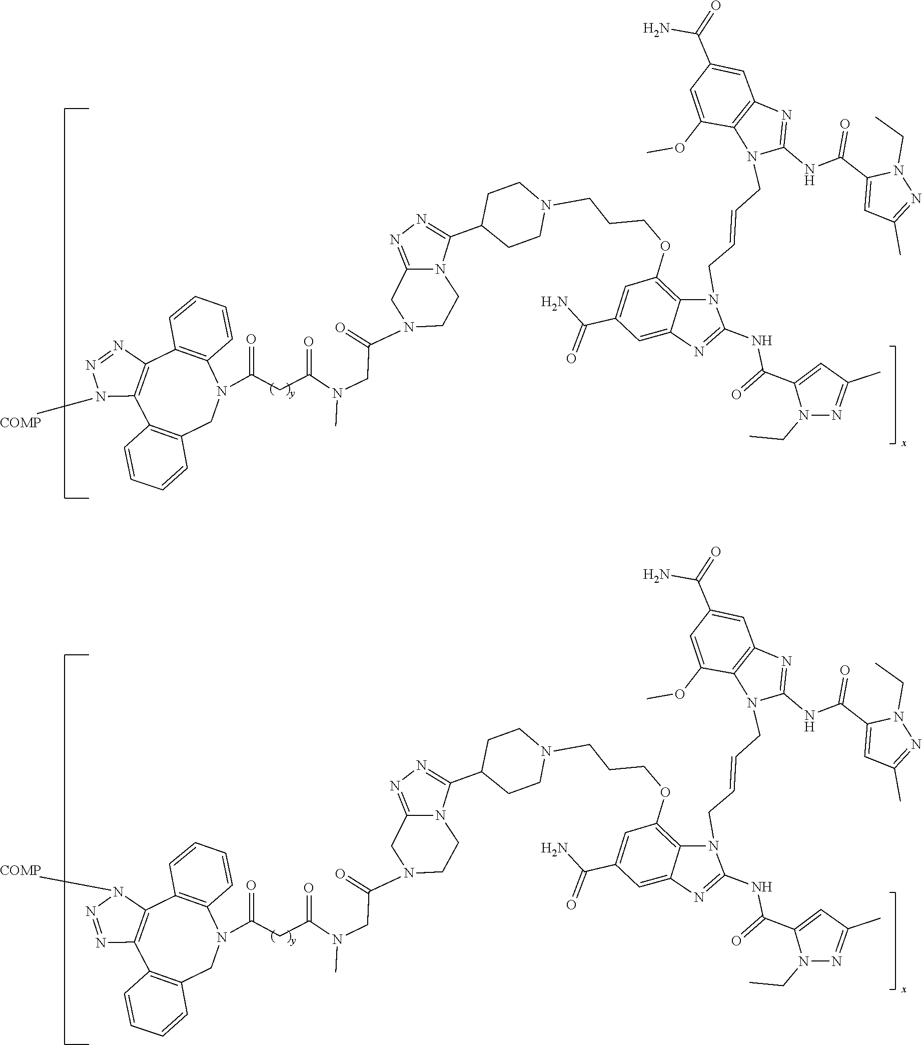 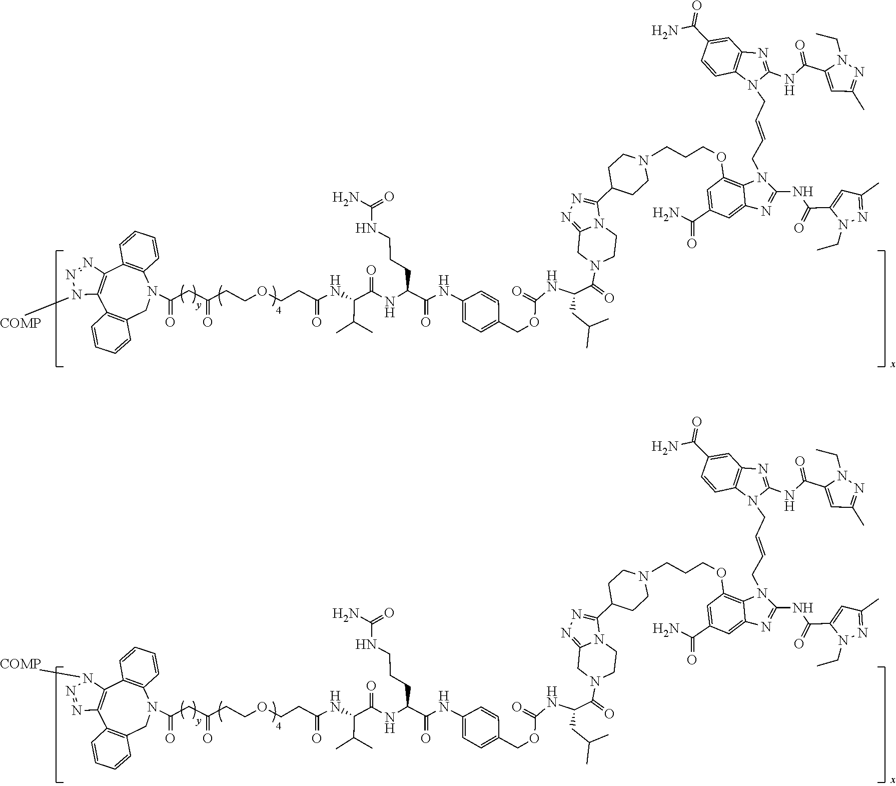
or a pharmaceutically acceptable salt or tautomer thereof, wherein x is an integer between 1 and 30, inclusive.

129. The conjugate of claim 63, wherein COMP is a polypeptide, an antibody or antigen binding fragment thereof, or an antibody chain.

130. (canceled)

131. (canceled)

132. The compound of claim 1 that is a pharmaceutically acceptable salt.

133. A pharmaceutical composition comprising a compound of claim 1 and a pharmaceutically acceptable excipient.

134. (canceled)

135. A method of treating a disease or disorder mediated by STING in a subject in need thereof comprising administering a compound of claim 1.

136. A method of inducing an immune response in a subject in need thereof comprising administering a compound of claim 1.

137. (canceled)

138. (canceled)

139. A method of treating abnormal cellular proliferation in a subject in need thereof comprising administering a compound of claim 1.

140-154. (canceled)
